(19) United States
(12) Patent Application Publication
(10) Pub. No.: US 2026/0224709 A1
(43) Pub. Date: Aug. 6, 2026

(54) C40-, C28-, and C-32-Linked Rapamycin Analogs as mTOR Inhibitors

(71) Applicant: Revolution Medicines, Inc., Redwood City, CA (US)

(72) Inventors: Jennifer Pitzen, Redwood City, CA (US); Micah James Evans Gliedt, Redwood City, CA (US); G. Leslie Burnett, Redwood City, CA (US); James Bradley Aggen, Redwood City, CA (US); Gert Kiss, Redwood City, CA (US); James Joseph Cregg, Redwood City, CA (US); Christopher Michael Semko, Redwood City, CA (US); Walter Won, Redwood City, CA (US); Gang Wang, Redwood City, CA (US); Julie Chu-Li Lee, Redwood City, CA (US); Arun P. Thottumkara, Redwood City, CA (US); Adrian Liam Gill, Redwood City, CA (US); Kevin T. Mellem, Redwood City, CA (US)

(21) Appl. No.: 19/312,078

(22) Filed: Aug. 27, 2025

Related U.S. Application Data
(63) Continuation of application No. 18/651,408, filed on Apr. 30, 2024, now Pat. No. 12,544,448.
Application No. 18/651,408 is a continuation of application No. 17/733,755, filed on Apr. 29, 2022, now Pat. No. 12,048,749.
Application No. 17/733,755 is a continuation of application No. 17/083,172, filed on Oct. 28, 2020, now Pat. No. 11,364,300.
Application No. 17/083,172 is a continuation of application No. 16/398,011, filed on Apr. 29, 2019, now abandoned.
(60) Provisional application No. 62/665,435, filed on May 1, 2018.
(60) Provisional application No. 62/752,874, filed on Oct. 30, 2018.
(60) Provisional application No. 62/836,036, filed on Apr. 18, 2019.

(51) Int. Cl. A61K 47/54; A61K 31/436; A61K 31/519; A61K 31/553

(52) U.S. Cl. CPC A61K 47/545; A61K 31/436; A61K 31/519; A61K 31/553

(57) ABSTRACT

The present disclosure relates to mTOR inhibitors. Specifically, the embodiments are directed to compounds and compositions inhibiting mTOR, methods of treating diseases mediated by mTOR, and methods of synthesizing these compounds.

### CROSS REFERENCE TO RELATED APPLICATIONS

This application is a continuation of U.S. application Ser. No. 18/651,408, filed Apr. 30, 2024, which is a continuation of U.S. application Ser. No. 17/733,755, filed Apr. 29, 2022, now U.S. Pat. No. 12,048,749, which is a continuation of U.S. application Ser. No. 17/083,172, filed Oct. 28, 2020, now U.S. Pat. No. 11,364,300, which is a continuation of U.S. application Ser. No. 16/398,011, filed Apr. 29, 2019, which claims the benefit of U.S. Provisional Application No. 62/665,435, filed May 1, 2018 and U.S. Provisional Application No. 62/752,874, filed Oct. 30, 2018 and U.S. Provisional Application No. 62/836,036, filed Apr. 18, 2019, the contents of which are incorporated herein by reference in their entireties.

### REFERENCE TO A SEQUENCE LISTING

The Sequence Listing associated with this application is provided in XML format in lieu of a paper copy, and is hereby incorporated by reference in its entirety into the specification. The name of the XML file containing the Sequence Listing is REME_008_08US_SeqList_ST26.xml. The XML file is about 17,341 bytes, was created on Aug. 26, 2025, and is being submitted electronically via Patent Center.

### FIELD OF THE DISCLOSURE

The present disclosure relates to mTOR inhibitors. Specifically, the embodiments are directed to compounds and compositions inhibiting mTOR, methods of treating diseases mediated by mTOR, and methods of synthesizing these compounds.

### BACKGROUND OF THE DISCLOSURE

The mammalian target of rapamycin (mTOR) is a serine-threonine kinase related to the lipid kinases of the phosphoinositide 3-kinase (PI3K) family. mTOR exists in two complexes, mTORC1 and mTORC2, which are differentially regulated, have distinct substrate specificities, and are differentially sensitive to rapamycin. mTORC1 integrates signals from growth factor receptors with cellular nutritional status and controls the level of cap-dependent mRNA translation by modulating the activity of key translational components such as the cap-binding protein and oncogene eIF4E.

mTOR signaling has been deciphered in increasing detail. The differing pharmacology of inhibitors of mTOR has been particularly informative. The first reported inhibitor of mTOR, Rapamycin is now understood to be an incomplete inhibitor of mTORC1. Rapamycin is a selective mTORC1 inhibitor through the binding to the FK506 Rapamycin Binding (FRB) domain of mTOR kinase with the aid of FK506 binding protein 12 (FKBP12). The FRB domain of mTOR is accessible in the mTORC1 complex, but less so in the mTORC2 complex. Interestingly, the potency of inhibitory activities against downstream substrates of mTORC1 by the treatment of Rapamycin is known to be diverse among the mTORC1 substrates. For example, Rapamycin strongly inhibits phosphorylation of the mTORC1 substrate S6K and, indirectly, phosphorylation of the downstream ribosomal protein S6 which control ribosomal biogenesis. On the other hand, Rapamycin shows only partial inhibitory activity against phosphorylation of 4E-BP1, a major regulator of eIF4E which controls the initiation of CAP-dependent translation. As a result, more complete inhibitors of mTORC1 signaling are of interest.

A second class of “ATP-site” inhibitors of mTOR kinase were reported. This class of mTOR inhibitors will be referred to as TORi (ATP site TOR inhibitor). The molecules compete with ATP, the substrate for the kinase reaction, in the active site of the mTOR kinase (and are therefore also mTOR active site inhibitors). As a result, these molecules inhibit downstream phosphorylation of a broader range of substrates.

Although mTOR inhibition may have the effect of blocking 4E-BP1 phosphorylation, these agents may also inhibit mTORC2, which leads to a block of Akt activation due to inhibition of phosphorylation of Akt 5473.

Disclosed herein, inter alia, are mTOR inhibitors. In some embodiments, compounds disclosed herein are more selective inhibitors of mTORC1 versus mTORC2. In some embodiments, compounds disclosed herein are more selective inhibitors of mTORC2 versus mTORC1. In some embodiments, compounds disclosed herein exhibit no selectivity difference between mTORC1 and mTORC2.

### SUMMARY OF THE DISCLOSURE

The present disclosure relates to compounds capable of inhibiting the activity of mTOR. The present disclosure further provides a process for the preparation of compounds of the present disclosure, pharmaceutical preparations comprising such compounds and methods of using such compounds and compositions in the management of diseases or disorders mediated by mTOR.

The present disclosure provides a compound of Formula Ic:

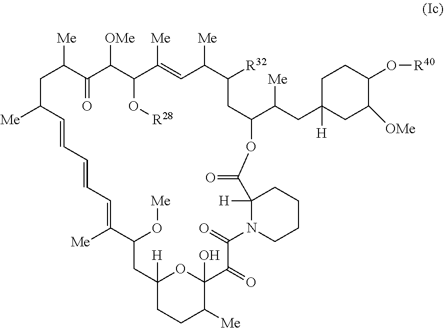

or a pharmaceutically acceptable salt or tautomer thereof, wherein:
R³² is —H, ═O, —OR³, —N₃, or —O—C(═Z¹)—R^{32a};
R²⁸ is —H, (C₁-C₆)alkyl, or —C(═Z¹)—R^{28a};
R⁴⁰ is —H or —C(═Z¹)—R^{40a};
   wherein when R²⁸ and R⁴⁰ are H, then R³² is not ═O;
each Z¹ is independently O or S;
R^{28a}, R^{32a}, and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;
A¹ and A² are independently absent or are independently selected from

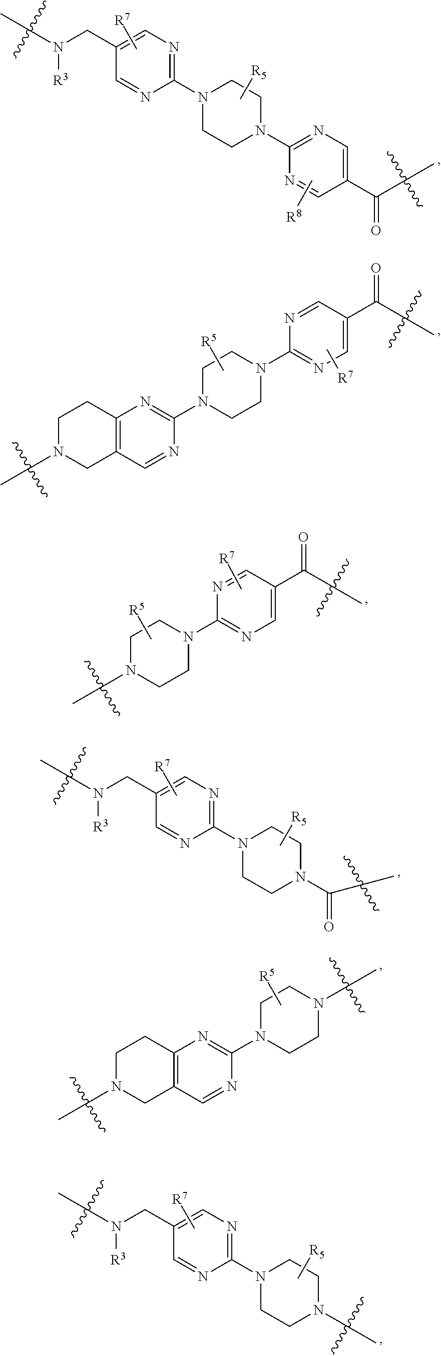

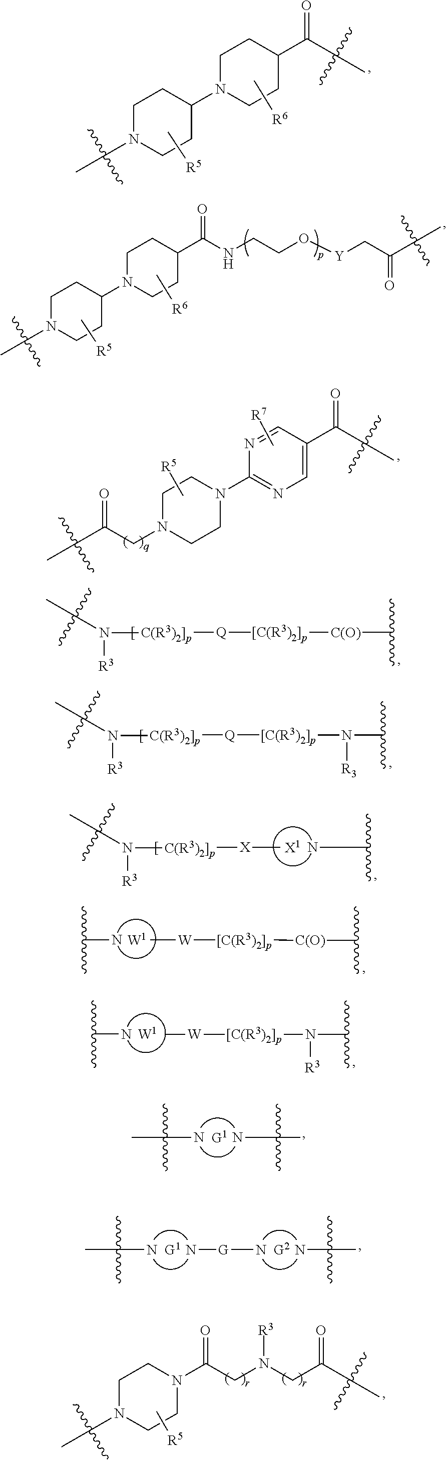

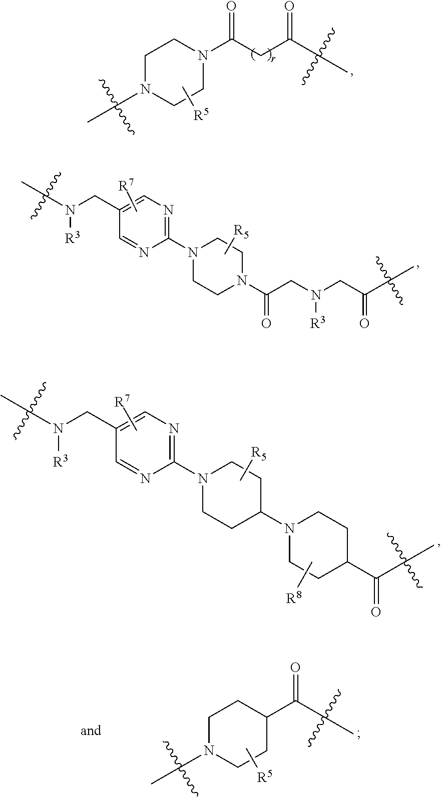

wherein the bond on the left side of A¹, as drawn, is bound to —C(═Z¹)— or L²; and wherein the bond on the right side of the A² moiety, as drawn, is bound to B or L³;
each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ is independently a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;
each L¹ is independently selected from

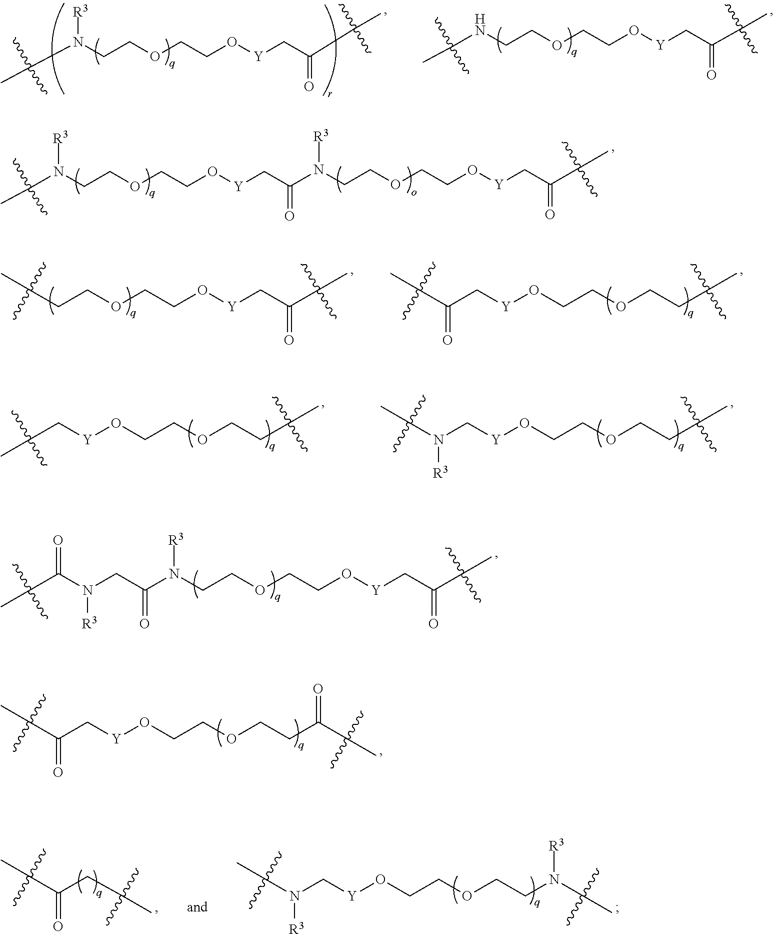

L² and L³ are independently absent or are independently selected from

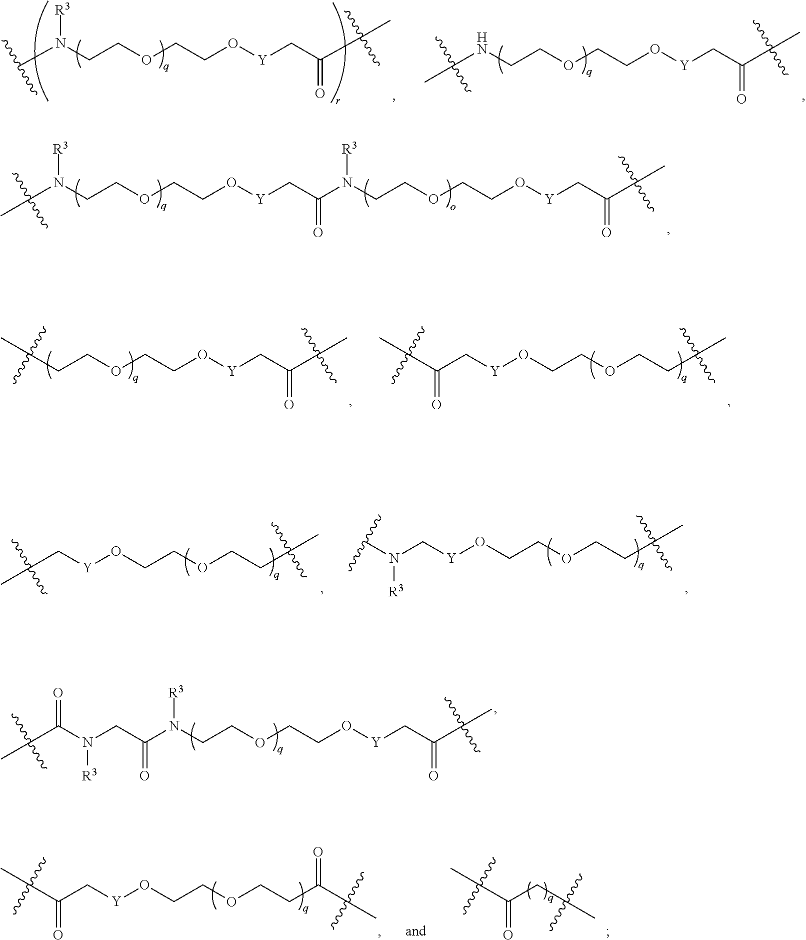

each B is independently selected from

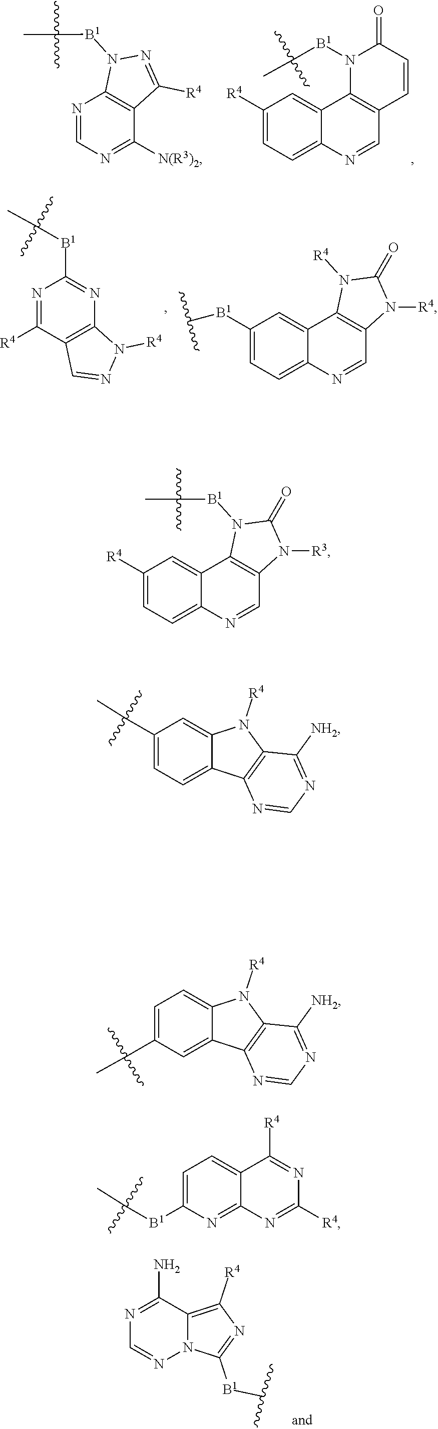

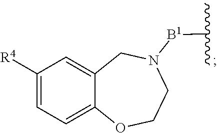

each B¹ is independently selected from

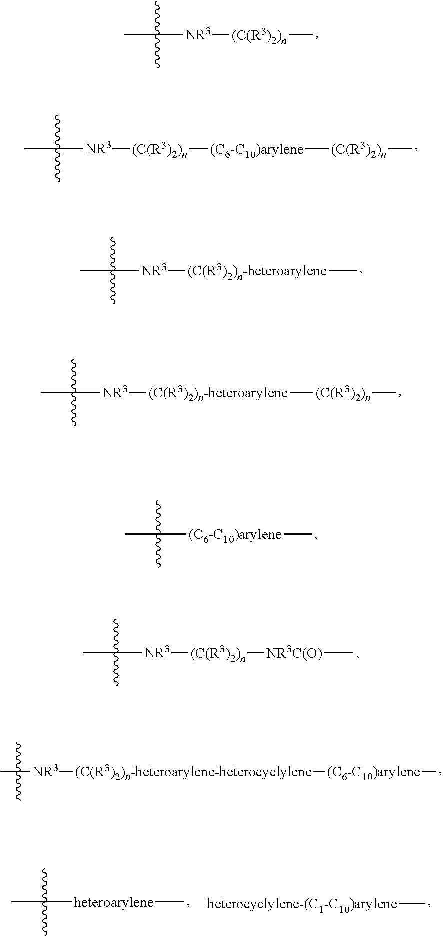

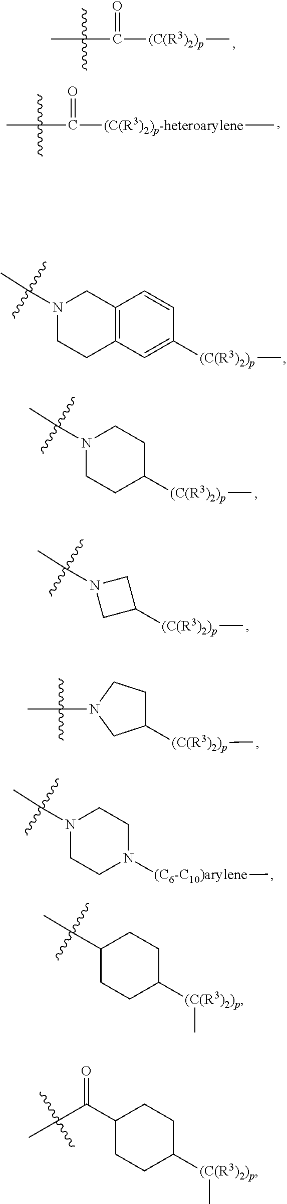

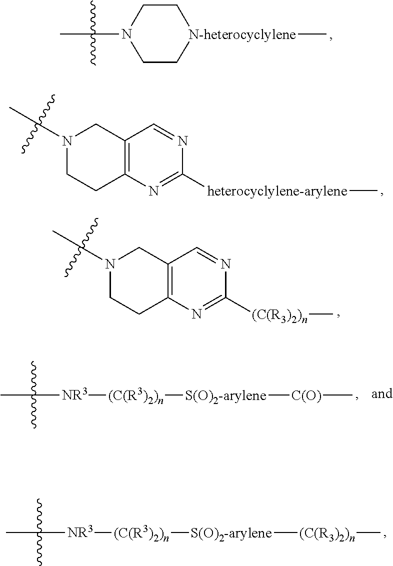

wherein the

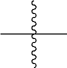

bond on the left side of B¹, as drawn, is bound to A², L³, or L¹; and wherein the heteroarylene, heterocyclylene, and arylene are each independently optionally substituted with alkyl, hydroxyalkyl, haloalkyl, alkoxy, halogen, or hydroxyl;
each R³ is independently H or (C₁-C₆)alkyl;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are each independently optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;
each R⁵ is independently H, (C₁-C₆)alkyl, —C(O)OR³, or —N(R³)₂, wherein the alkyl of (C₁-C₆)alkyl is each independently optionally substituted with —N(R³)₂ or —OR³;
each R⁶ is independently H, (C₁-C₆)alkyl, —C(O)OR³, or —N(R³)₂, wherein the alkyl of (C₁-C₆)alkyl is each independently optionally substituted with —N(R³)₂ or —OR³;
each R⁷ is independently H, (C₁-C₆)alkyl, —C(O)OR³, or —N(R³)₂, wherein the alkyl of (C₁-C₆)alkyl is each independently optionally substituted with —N(R³)₂ or —OR³;
each R⁸ is independently H, (C₁-C₆)alkyl, —C(O)OR³, or —N(R³)₂, wherein the alkyl of (C₁-C₆)alkyl is each independently optionally substituted with —N(R³)₂ or —OR³;
each Y is independently —C(R³)₂ or a bond;
each n is independently an integer from one to 12;
each o is independently an integer from zero to 30;
each p is independently an integer from zero to 12;
each q is independently an integer from zero to 30; and
each r is independently an integer from one to 6.

The present disclosure provides a compound of Formula Ia:

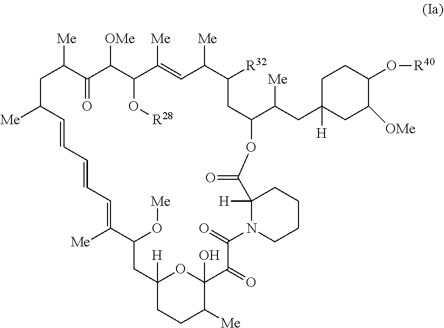

or a pharmaceutically acceptable salt or tautomer thereof, wherein:
R³² is —H, ═O, —OR³, —N₃, or —O—C(═Z¹)—R^{32a};
R²⁸ is —H, (C₁-C₆)alkyl, or —C(═Z¹)—R^{28a};
R⁴⁰ is —H or —C(═Z¹)—R^{40a};
   wherein when R²⁸ and R⁴⁰ are H, then R³² is not ═O;
each Z¹ is independently O or S;
R^{28a}, R^{32a}, and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;
A¹ and A² are independently absent or are independently selected from

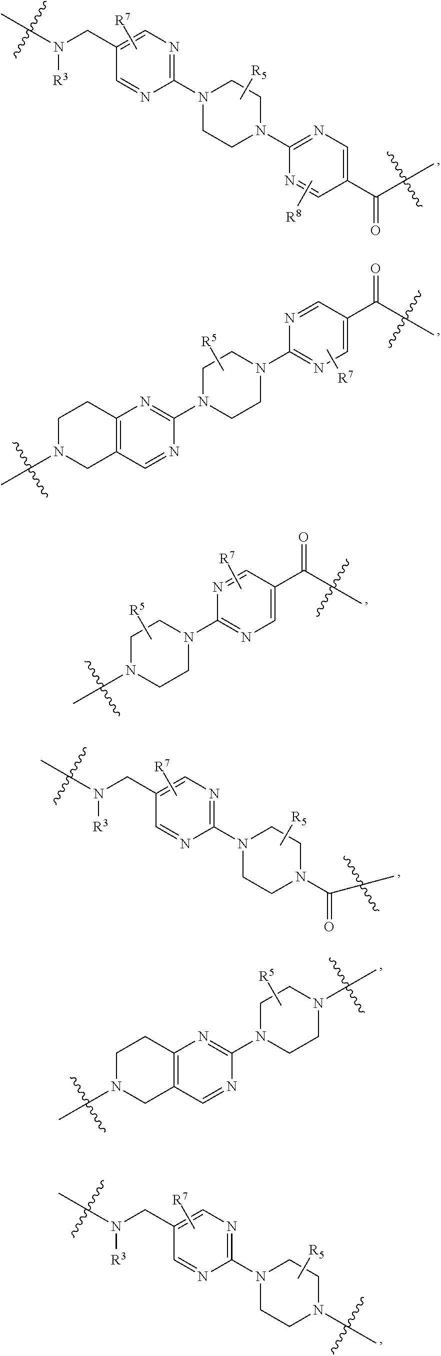

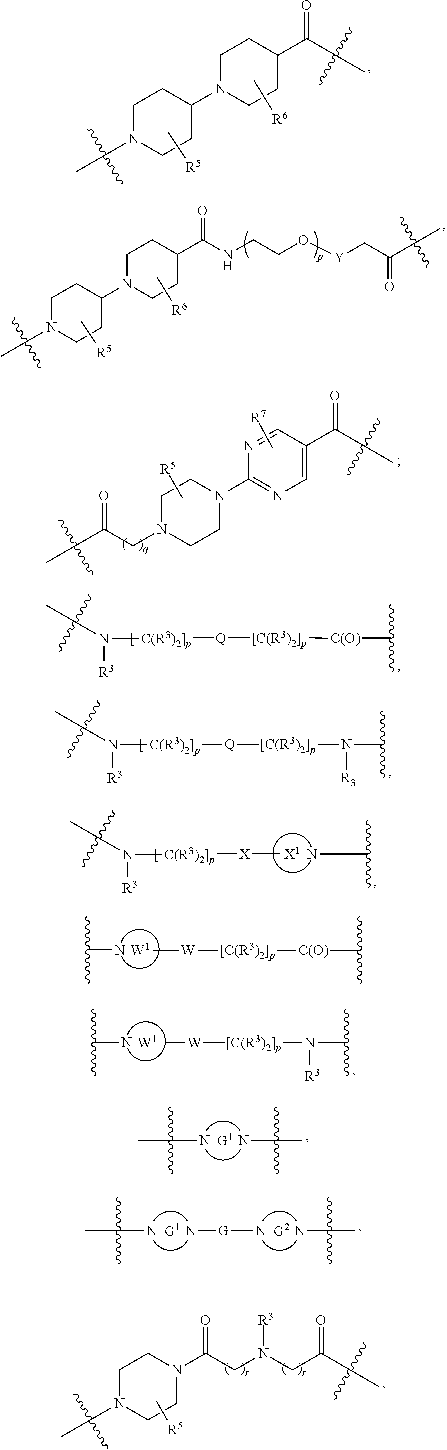

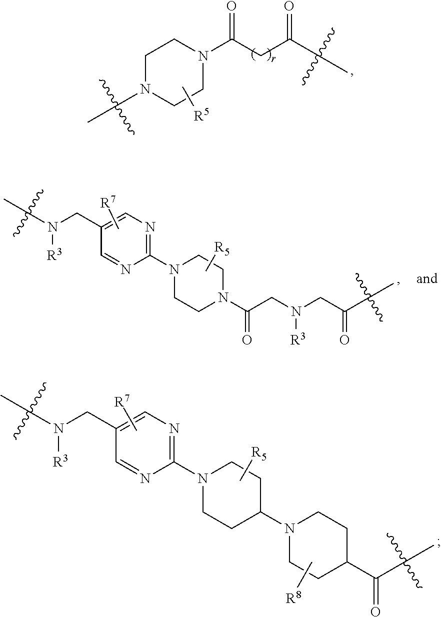

wherein the bond on the left side of A¹, as drawn, is bound to —C(═Z¹)— or L²; and wherein the bond on the right side of the A² moiety, as drawn, is bound to B or L³.
each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ independently is a heteroarylene or heterocyclylene ring;
each W is independently absent or 1 to 2 rings selected from arylene, cycloalkylene, heteroarylene, and heterocyclylene;
each W¹ independently is a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;
each L¹ is independently selected from

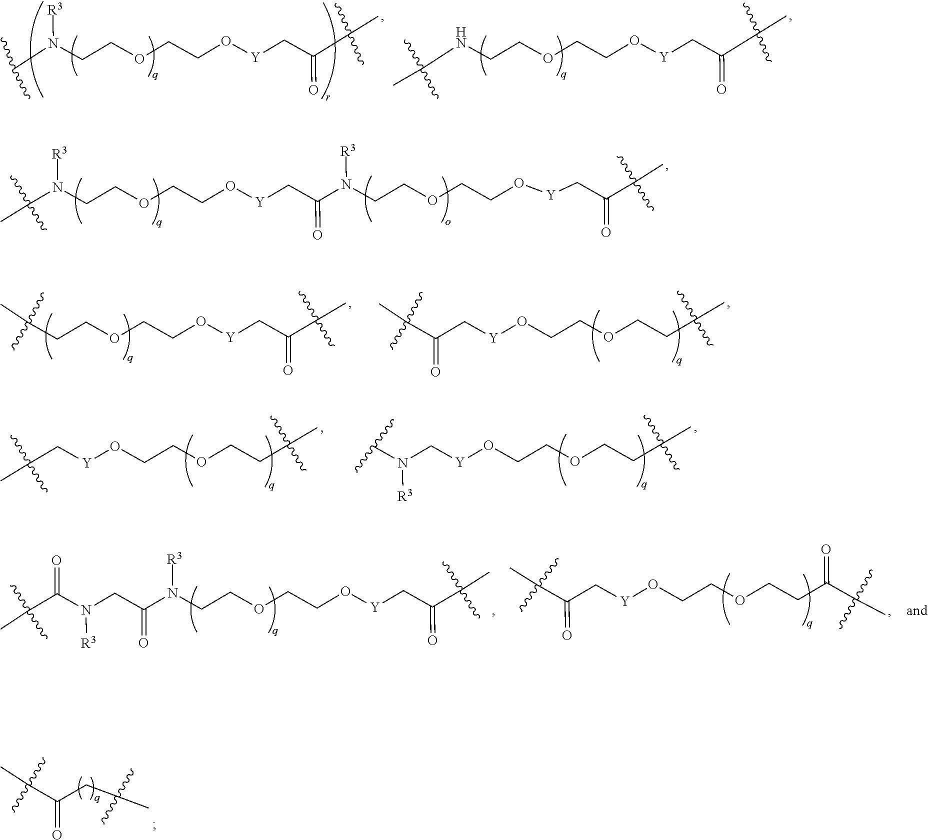

L² and L³ are independently absent or are independently selected from

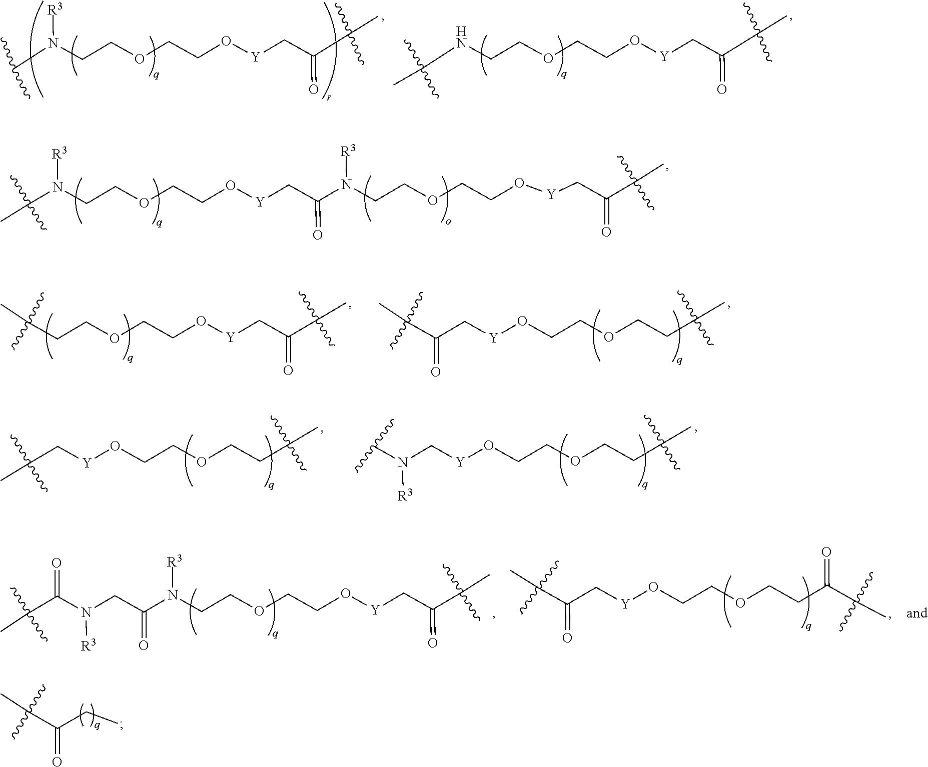

each B is independently selected from

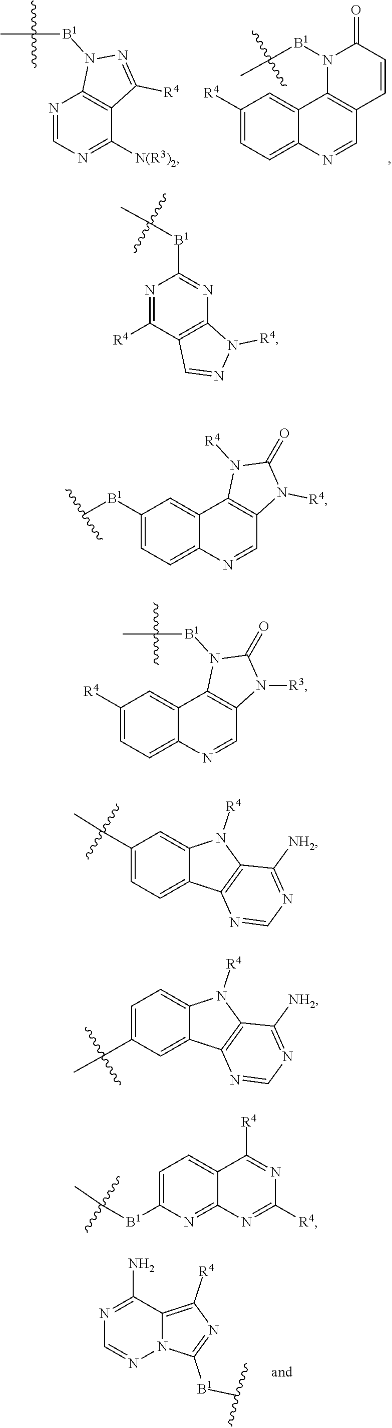

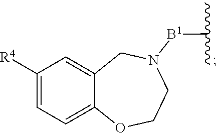

each B¹ is independently selected from

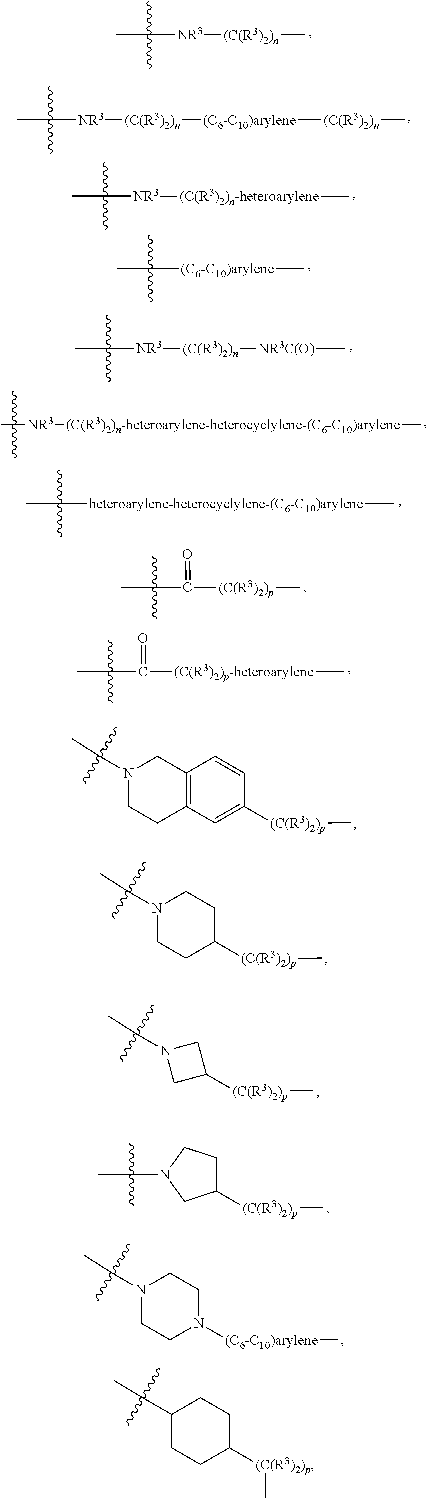

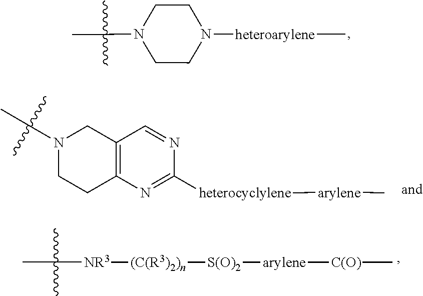

wherein the

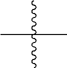

bond on the left side of B¹, as drawn, is bound to A², L³, or L¹; and wherein the heteroarylene, heterocyclylene, and arylene are each independently optionally substituted with alkyl, hydroxyalkyl, haloalkyl, alkoxy, halogen, or hydroxyl;
each R³ is independently H or (C₁-C₆)alkyl;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are each independently optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;
each R⁵ is independently H, (C₁-C₆)alkyl, —C(O)OR³, or —N(R³)₂, wherein the alkyl of (C₁-C₆)alkyl is each independently optionally substituted with —N(R³)₂ or —OR³;
each R⁶ is independently H, (C₁-C₆)alkyl, —C(O)OR³, or —N(R³)₂, wherein the alkyl of (C₁-C₆)alkyl is each independently optionally substituted with —N(R³)₂ or —OR³;
each R⁷ is independently H, (C₁-C₆)alkyl, —C(O)OR³, or —N(R³)₂, wherein the alkyl of (C₁-C₆)alkyl is each independently optionally substituted with —N(R³)₂ or —OR³;
each R⁸ is independently H, (C₁-C₆)alkyl, —C(O)OR³, or —N(R³)₂, wherein the alkyl of (C₁-C₆)alkyl is each independently optionally substituted with —N(R³)₂ or —OR³;
each Y is independently —C(R³)₂ or a bond;
each n is independently an integer from one to 12;
each o is independently an integer from zero to 30;
each p is independently an integer from zero to 12;
each q is independently an integer from zero to 30; and
each r is independently an integer from one to 6.

The present disclosure provides a compound of Formula I:

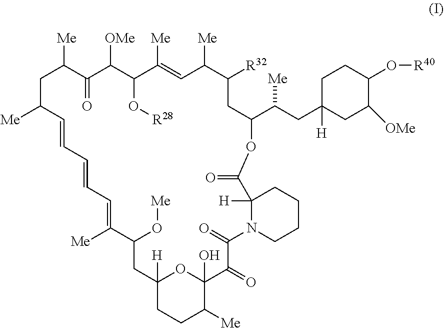

or pharmaceutically acceptable salt or tautomer thereof, wherein:
R³² is —H, ═O, or —OR³;
R²⁸ is —H or —C(═Z¹)—R^{28a};
R⁴⁰ is —H or —C(═Z¹)—R^{40a};
   wherein at least one of R²⁸ and R⁴⁰ is not H;
Z¹ is independently O or S;
R^{28a} and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;
A¹ and A² are independently absent or are independently selected from

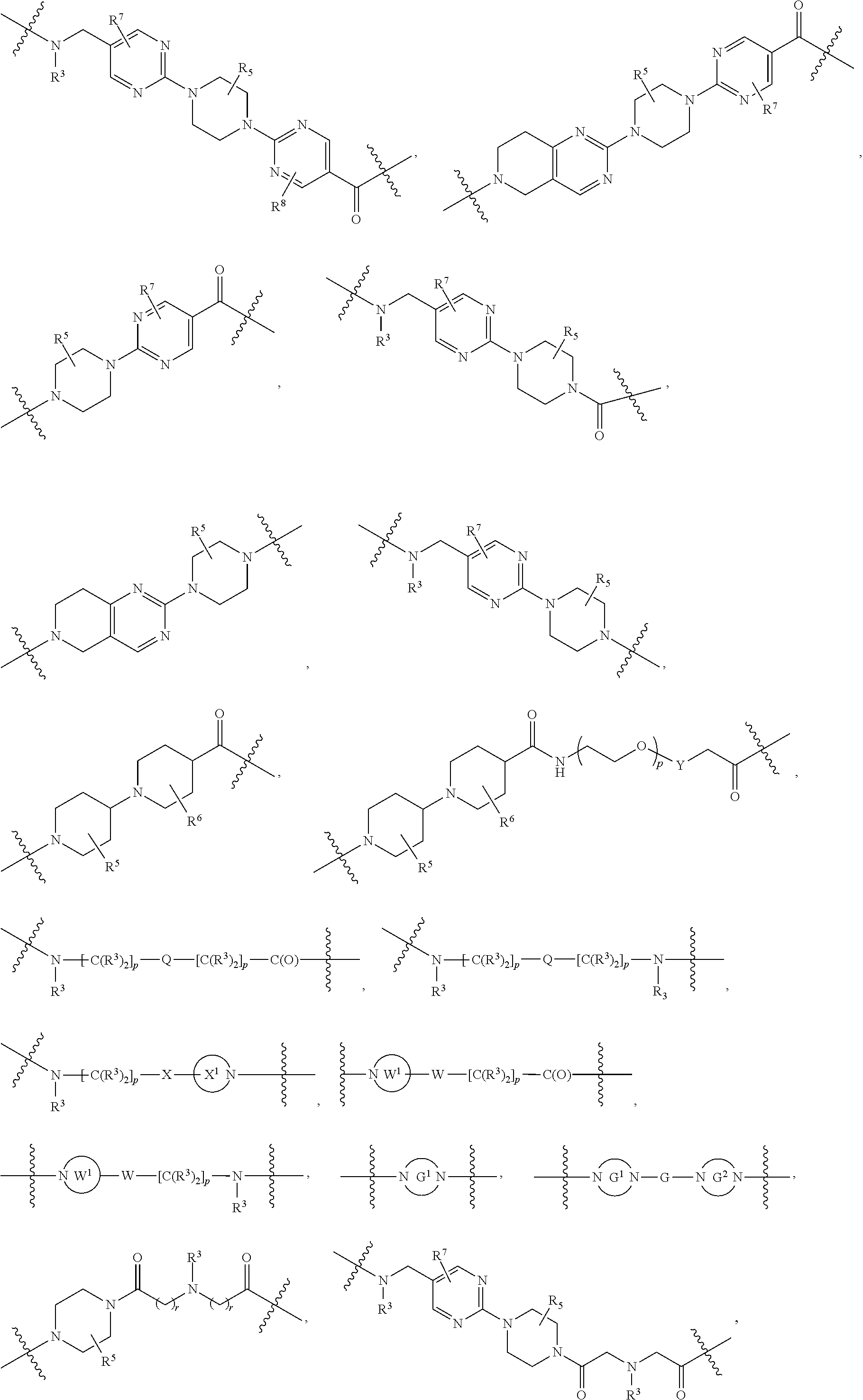

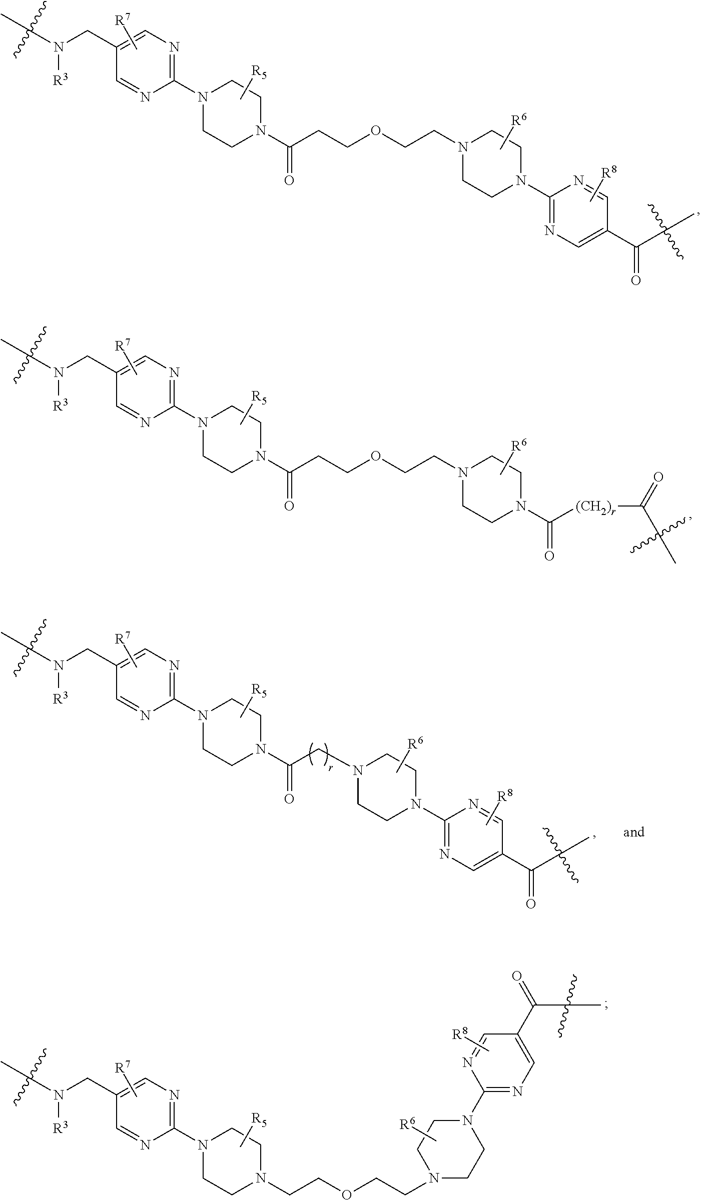

wherein the bond on the left side of A¹, as drawn, is bound to —C(═Z¹)— or L²; and wherein the bond on the right side of the A² moiety, as drawn, is bound to B or L³;
each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ independently is a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;
each L¹ is independently selected from

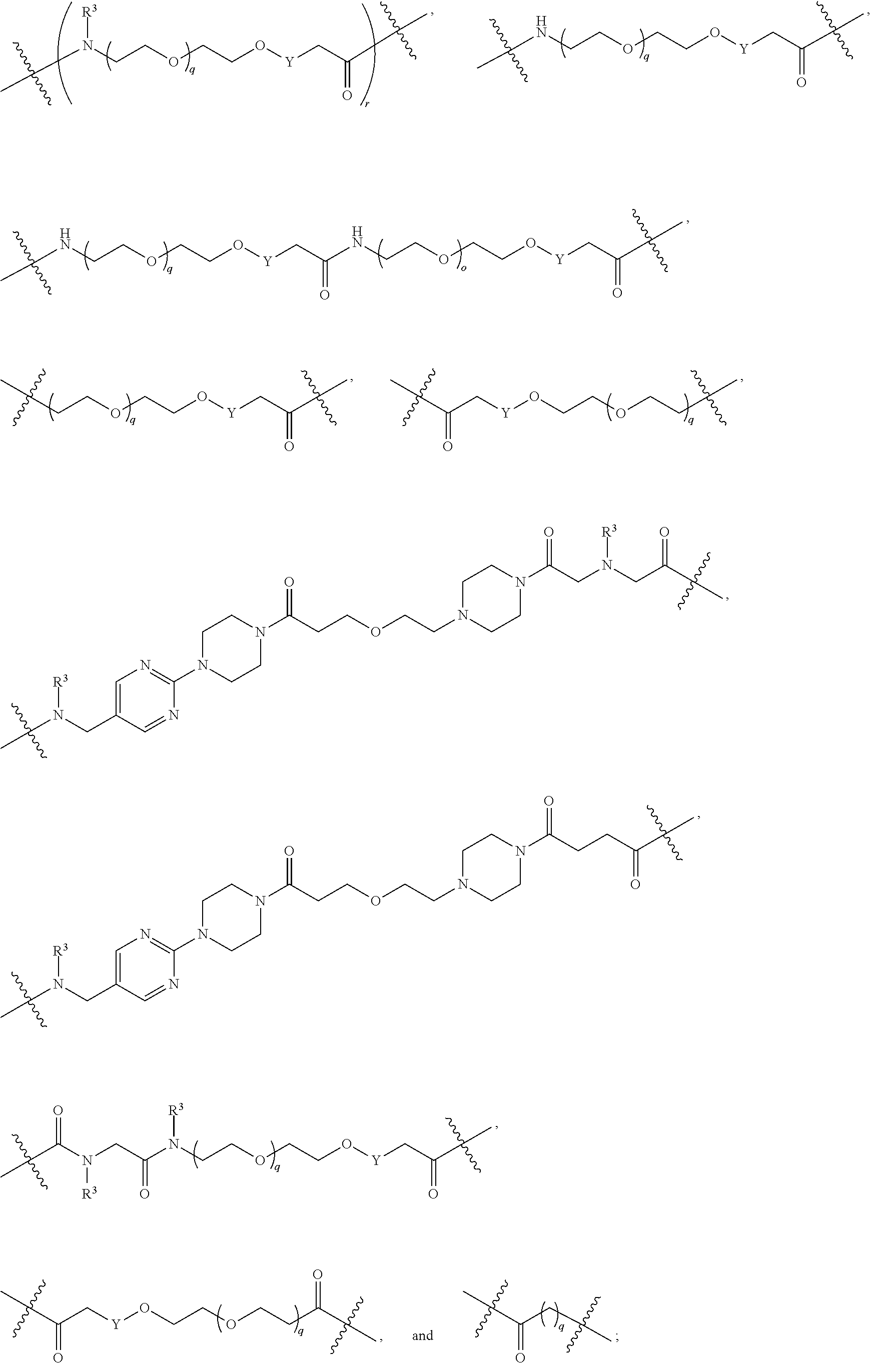

L² and L³ are independently absent or are independently selected from

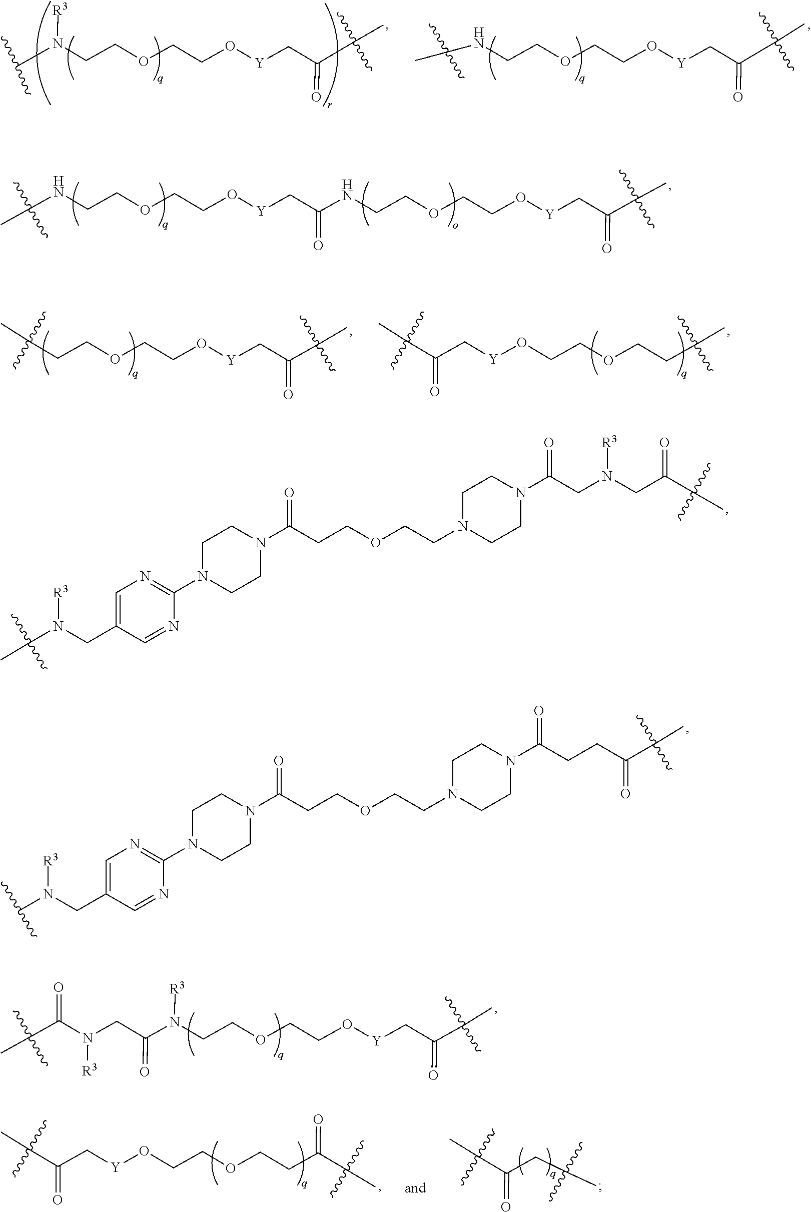

each B is independently selected from

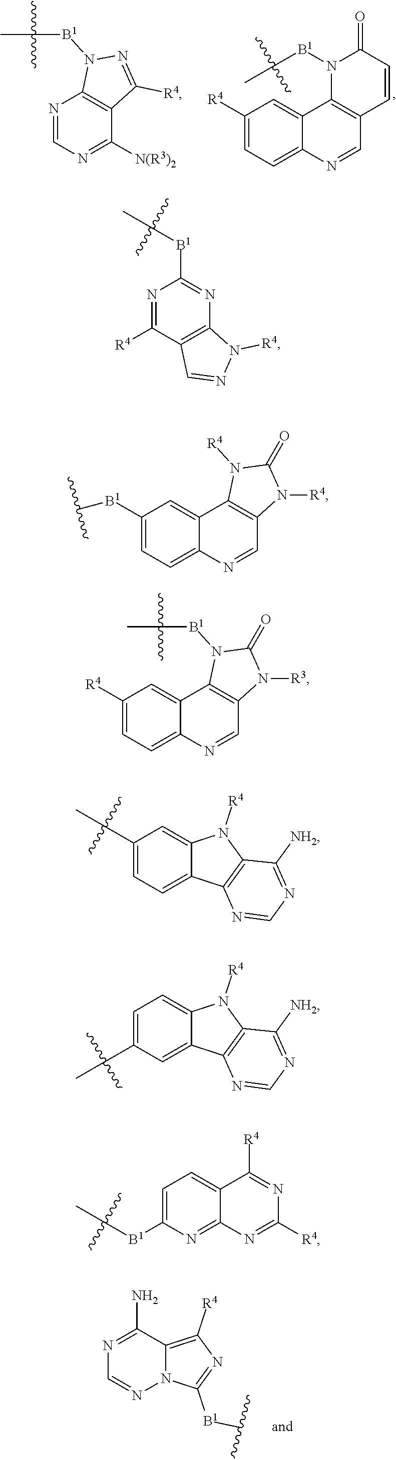

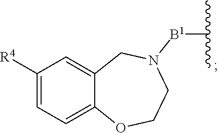

each B¹ is independently selected from

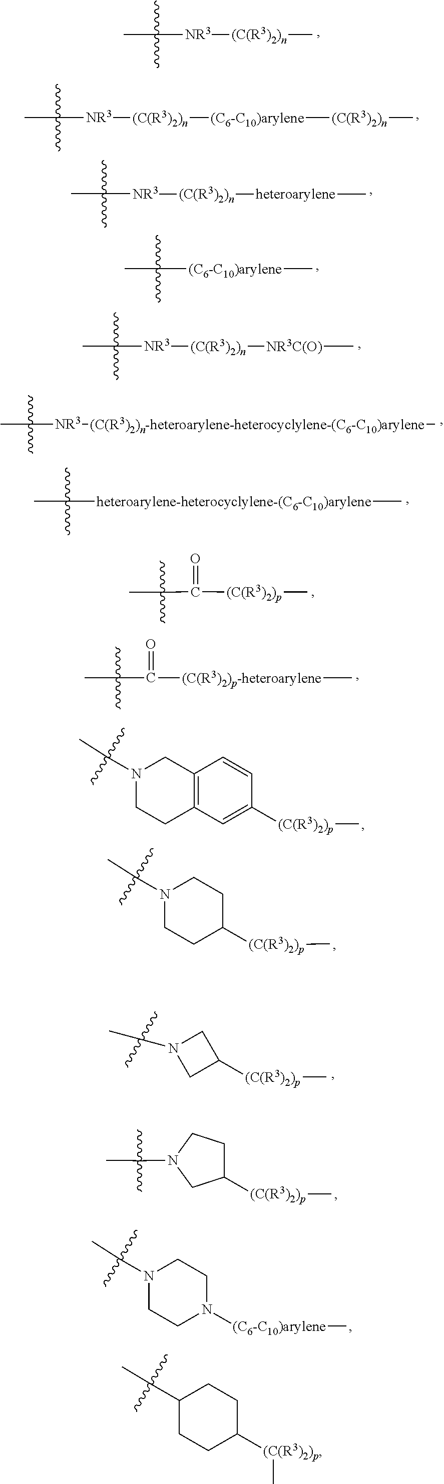

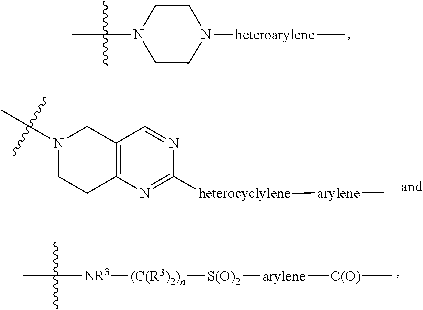

wherein the

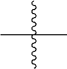

bond on the left side of b¹, as drawn, is bound to A², L³, or L¹; and wherein the heteroarylene, heterocyclylene, and arylene are each independently optionally substituted with alkyl, hydroxyalkyl, haloalkyl, alkoxy, halogen, or hydroxyl;
each R³ is independently H or (C₁-C₆)alkyl;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are each independently optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;
each R⁵ is independently H, (C₁-C₆)alkyl, —C(O)OR³, or —N(R³)₂, wherein the alkyl of (C₁-C₆)alkyl is optionally substituted with —N(R³)₂ or —OR³;
each R⁶ is independently H, (C₁-C₆)alkyl, —C(O)OR³, or —N(R³)₂, wherein the alkyl is of (C₁-C₆)alkyl optionally substituted with —N(R³)₂ or —OR³;
each R⁷ is independently H, (C₁-C₆)alkyl, —C(O)OR³, or —N(R³)₂, wherein the alkyl of (C₁-C₆)alkyl is optionally substituted with —N(R³)₂ or —OR³;
each R⁸ is independently H, (C₁-C₆)alkyl, —C(O)OR³, or —N(R³)₂, wherein the alkyl of (C₁-C₆)alkyl is optionally substituted with —N(R³)₂ or —OR³;
each Y is independently —C(R³)₂ or a bond;
each n is independently an integer from one to 12;
each o is independently an integer from zero to 30;
each p is independently an integer from zero to 12;
each q is independently an integer from zero to 30; and
each r is independently an integer from one to 6.

The present disclosure provides a compound of Formula II:

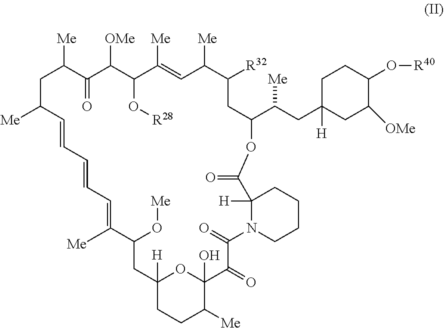

or a pharmaceutically acceptable salt or tautomer thereof, wherein:
R³² is —H, ═O or —OR³;
R²⁸ is —H or —C(═Z¹)—R^{28a};
R⁴⁰ is —H or —C(═Z¹)—R^{40a};
   wherein at least one of R²⁸ and R⁴⁰ is not H;
Z¹ is independently O or S;
R^{28a} and R^{40a} are independently -A¹-L¹-A²-B; -A¹-A²-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;
A¹ and A² are independently absent or are independently selected from

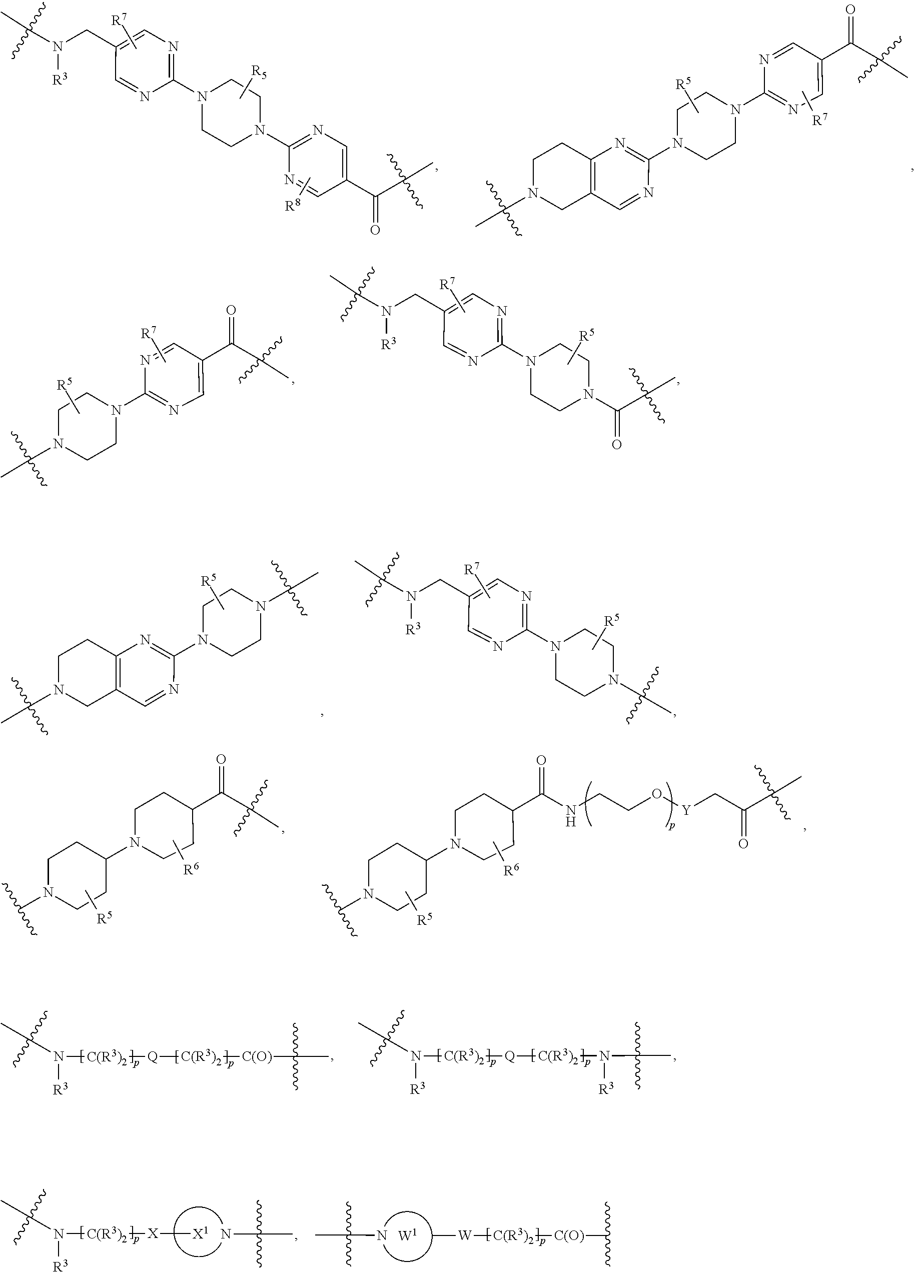

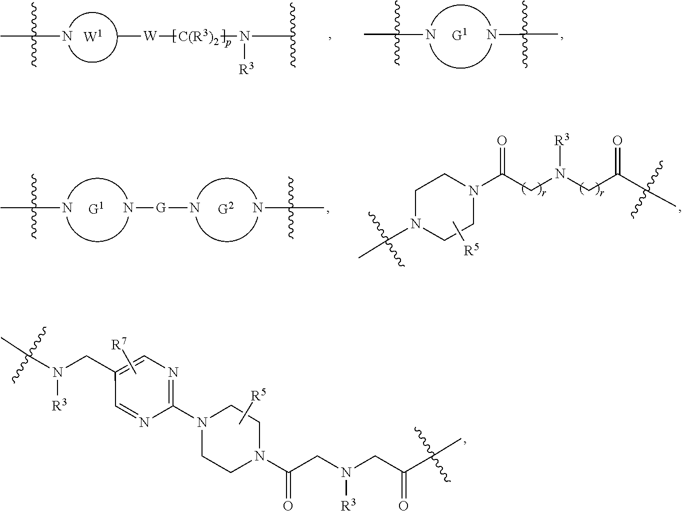

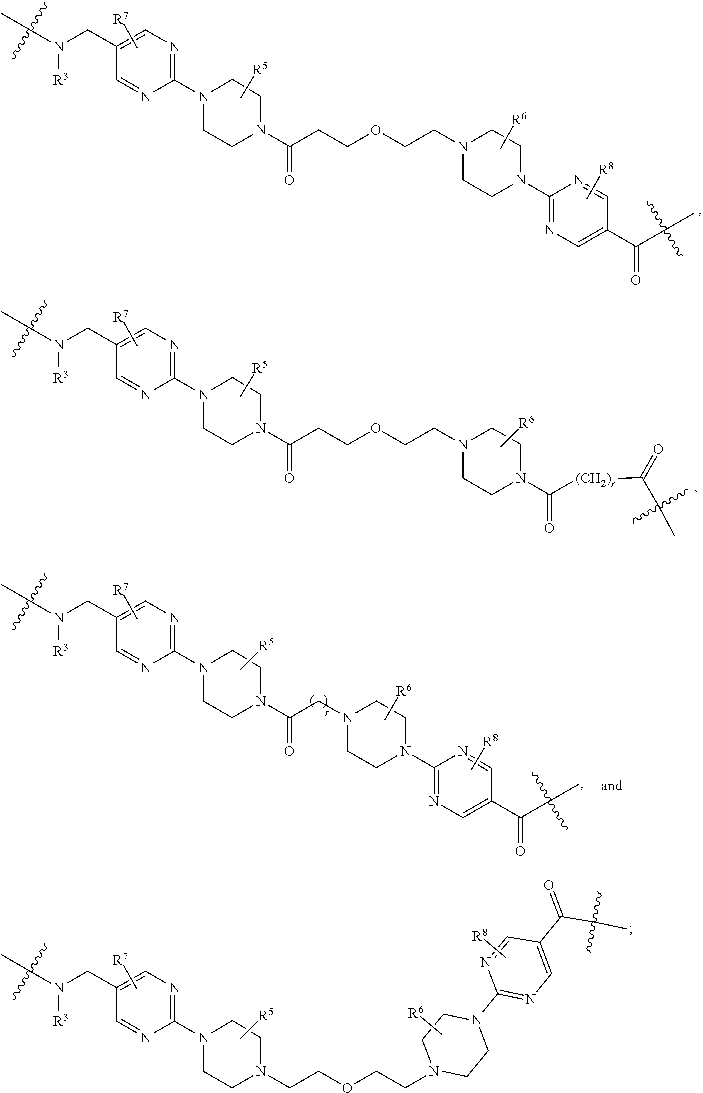

wherein the bond on the left side of A¹, as drawn, is bound to —C(═Z¹)—; and wherein the bond on the right side of the A² moiety, as drawn, is bound to B.
each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ is independently a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;
each L¹ is independently selected from

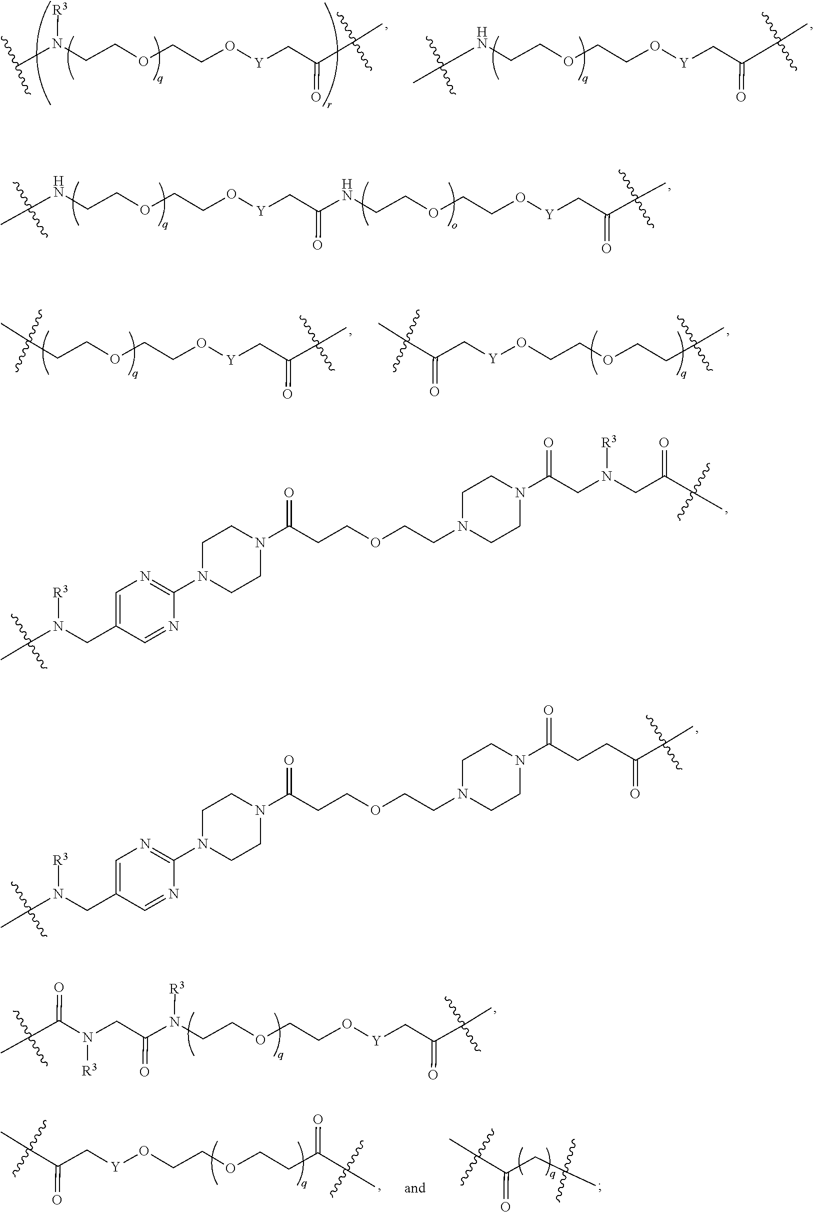

each B is independently selected from

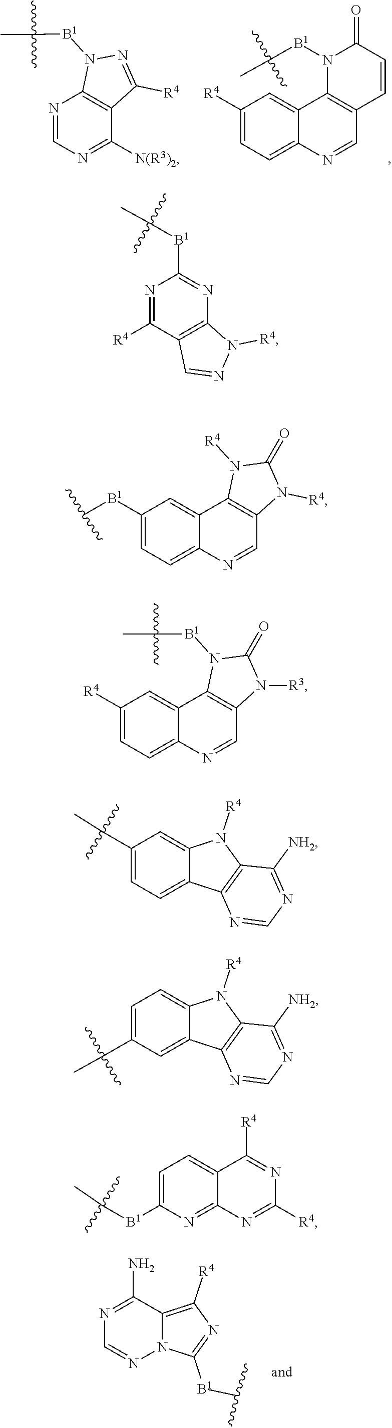

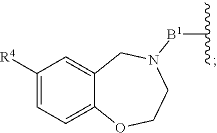

each B¹ is independently selected from

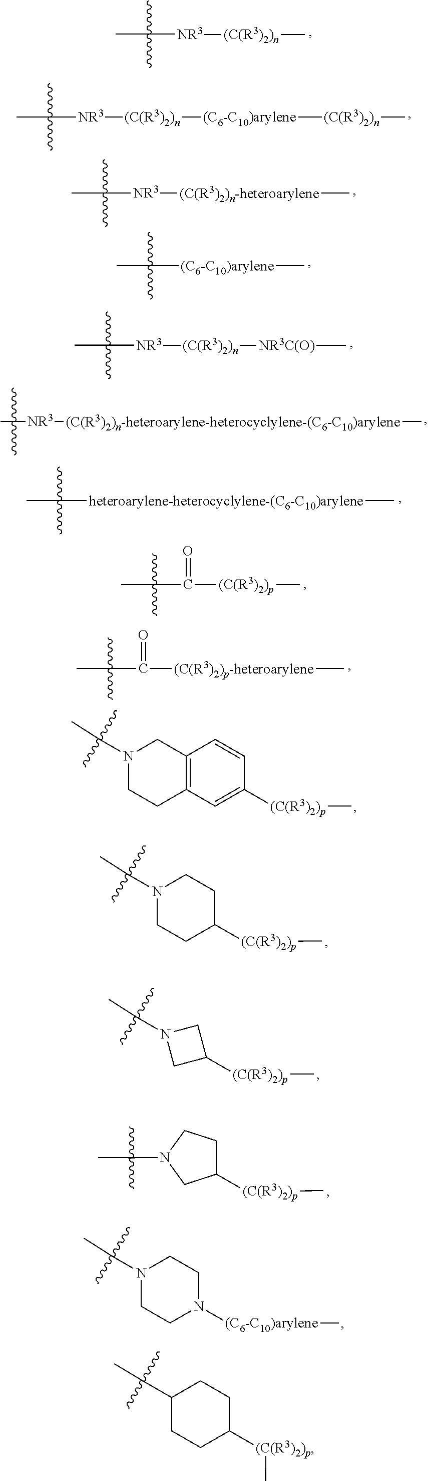

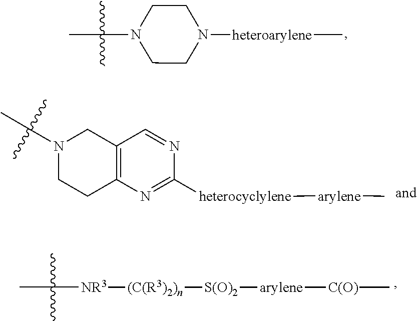

wherein the

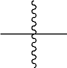

bond on the left side of B¹, as drawn, is bound to A² or L¹; and wherein the heteroarylene, heterocyclylene, and arylene are each independently optionally substituted with alkyl, hydroxyalkyl, haloalkyl, alkoxy, halogen, or hydroxyl;
each R³ is independently H or (C₁-C₆)alkyl;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are each independently optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;
each R⁵ is independently H, (C₁-C₆)alkyl, —C(O)OR³, or —N(R³)₂, wherein the alkyl of (C₁-C₆)alkyl is optionally substituted with —N(R³)₂ or —OR³;
each R⁶ is independently H, (C₁-C₆)alkyl, —C(O)OR³, or —N(R³)₂, wherein the alkyl of (C₁-C₆)alkyl is optionally substituted with —N(R³)₂ or —OR³;
each R⁷ is independently H, (C₁-C₆)alkyl, —C(O)OR³, or —N(R³)₂, wherein the alkyl of (C₁-C₆)alkyl is optionally substituted with —N(R³)₂ or —OR³;
each R⁸ is independently H, (C₁-C₆)alkyl, —C(O)OR³, or —N(R³)₂, wherein the alkyl of (C₁-C₆)alkyl is optionally substituted with —N(R³)₂ or —OR³;
each Y is independently C(R³)₂ or a bond;
each n is independently an integer from one to 12;
each o is independently an integer from zero to 30;
each p is independently an integer from zero to 12;
each q is independently an integer from zero to 30; and
each r is independently an integer from one to 6.

In some embodiments, a compound of Formula I or II is represented by the structure of Formula I-28:

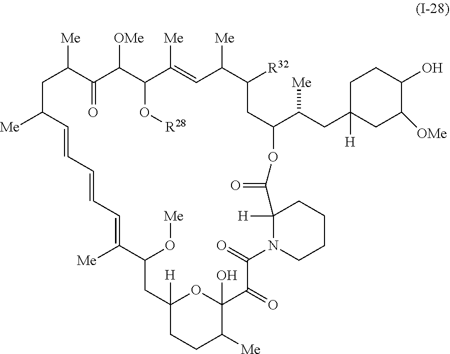

or a pharmaceutically acceptable salt or tautomer thereof.

In some embodiments, a compound of Formula Ia, Ic, I, or II is represented by the structure of Formula I-28b.

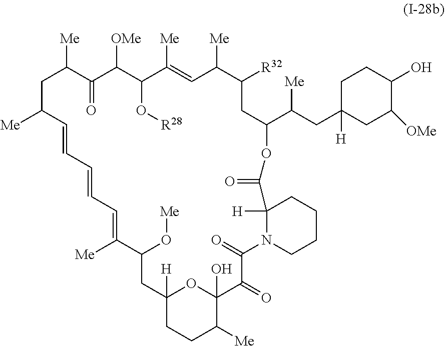

or a pharmaceutically acceptable salt or a tautomer thereof.

In some embodiments, a compound of Formula I or II is represented by the structure of Formula I-40:

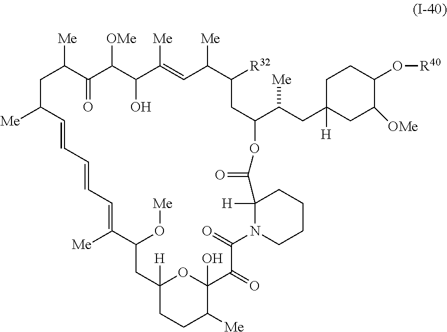

or a pharmaceutically acceptable salt or a tautomer thereof.

In some embodiments, a compound of Formula Ia, Ic, I or II is represented by the structure of Formula I-40b:

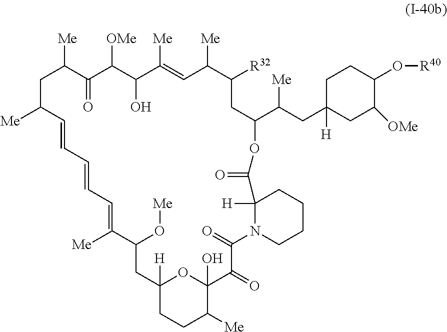

or a pharmaceutically acceptable salt or a tautomer thereof.

In some embodiments, a compound of Formula Ia, Ic, I or II is represented by the structure of Formula I-40b:

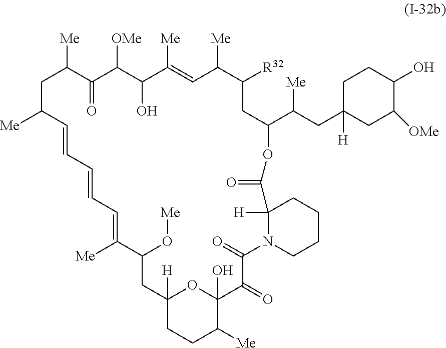

or a pharmaceutically acceptable salt or a tautomer thereof.

The present disclosure provides a method of treating a disease or disorder mediated by mTOR comprising administering to the subject suffering from or susceptible to developing a disease or disorder mediated by mTOR a therapeutically effective amount of one or more disclosed compounds. The present disclosure provides a method of preventing a disease or disorder mediated by mTOR comprising administering to the subject suffering from or susceptible to developing a disease or disorder mediated by mTOR a therapeutically effective amount of one or more disclosed compounds. The present disclosure provides a method of reducing the risk of a disease or disorder mediated by mTOR comprising administering to the subject suffering from or susceptible to developing a disease or disorder mediated by mTOR a therapeutically effective amount of one or more disclosed compounds.

Another aspect of the present disclosure is directed to a pharmaceutical composition comprising a compound of Formula I, Ia, Ib, Ic, II, or IIb, or a pharmaceutically acceptable salt or tautomer of any of the foregoing, and a pharmaceutically acceptable carrier. The pharmaceutically acceptable carrier can further comprise an excipient, diluent, or surfactant. The pharmaceutical composition can be effective for treating, preventing, or reducing the risk of a disease or disorder mediated by mTOR in a subject in need thereof.

Another aspect of the present disclosure relates to a compound of Formula I, Ia, Ib, Ic, II, or IIb, or a pharmaceutically acceptable salt or tautomer of any of the foregoing, for use in treating, preventing, or reducing the risk of a disease or disorder mediated by mTOR in a subject in need thereof.

Another aspect of the present disclosure relates to the use of a compound of Formula I, Ia, Ib, Ic, II, or IIb, or a pharmaceutically acceptable salt or tautomer of any of the foregoing, in the manufacture of a medicament for in treating, preventing, or reducing the risk of a disease or disorder mediated by mTOR in a subject in need thereof.

The present disclosure also provides compounds that are useful in inhibiting mTOR.

### DETAILED DESCRIPTION OF THE DISCLOSURE

The present disclosure relates to mTOR inhibitors. Specifically, the embodiments are directed to compounds and compositions inhibiting mTOR, methods of treating diseases mediated by mTOR, and methods of synthesizing these compounds.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also may include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Terms

The articles “a” and “an” are used in this disclosure and refers to one or more than one (i.e., to at least one) of the grammatical object of the article, unless indicated otherwise. By way of example, “an element” may mean one element or more than one element, unless indicated otherwise.

The term “or” means “and/or” unless indicated otherwise. The term “and/or” means either “and” or “or”, or both, unless indicated otherwise.

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment an optionally substituted group has 2 substituents. In another embodiment an optionally substituted group has 3 substituents. In another embodiment an optionally substituted group has 4 substituents. In another embodiment an optionally substituted group has 5 substituents.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene,” by itself or as part of another substituent, means, unless otherwise stated, a divalent radical derived from an alkyl. Typically, an alkyl (or alkylene) group will have from 1 to 24 carbon atoms, such as those groups having 10 or fewer carbon atoms.

The term “alkenyl” means an aliphatic hydrocarbon group containing a carbon carbon double bond and which may be straight or branched having about 2 to about 6 carbon atoms in the chain. Certain alkenyl groups have 2 to about 4 carbon atoms in the chain. Branched may mean that one or more lower alkyl groups such as methyl, ethyl, or propyl are attached to a linear alkenyl chain. Exemplary alkenyl groups include ethenyl, propenyl, n-butenyl, and i-butenyl. A C₂-C₆ alkenyl group is an alkenyl group containing between 2 and 6 carbon atoms.

The term “alkenylene,” by itself or as part of another substituent, means, unless otherwise stated, a divalent radical derived from an alkene.

The term “alkynyl” means an aliphatic hydrocarbon group containing a carbon carbon triple bond and which may be straight or branched having about 2 to about 6 carbon atoms in the chain. Certain alkynyl groups have 2 to about 4 carbon atoms in the chain. Branched may mean that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. A C₂-C₆ alkynyl group is an alkynyl group containing between 2 and 6 carbon atoms.

The term “alkynylene,” by itself or as part of another substituent, means, unless otherwise stated, a divalent radical derived from an alkyne.

The term “cycloalkyl” means a monocyclic or polycyclic saturated or partially unsaturated carbon ring containing 3-18 carbon atoms. Examples of cycloalkyl groups include, without limitation, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl. A C₃-C₈ cycloalkyl is a cycloalkyl group containing between 3 and 8 carbon atoms. A cycloalkyl group can be fused (e.g., decalin) or bridged (e.g., norbornane).

A “cycloalkylene,” alone or as part of another substituent, means a divalent radical derived from a cycloalkyl.

The terms “heterocyclyl” or “heterocycloalkyl” or “heterocycle” refers to a monocyclic or polycyclic 3 to 24-membered ring containing carbon and at least one heteroatom selected from oxygen, phosphorous, nitrogen, and sulfur and wherein there is not delocalized π electrons (aromaticity) shared among the ring carbon or heteroatom(s). Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heterocyclyl or heterocycloalkyl ring can also be fused or bridged, e.g., can be a bicyclic ring.

A “heterocyclylene” or “heterocycloalkylene,” alone or as part of another substituent, means a divalent radical derived from a “heterocyclyl” or “heterocycloalkyl” or “heterocycle.”

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl may refer to multiple rings fused together wherein at least one of the fused rings is an aryl ring.

An “arylene,” alone or as part of another substituent, means a divalent radical derived from an aryl.

The term “heteroaryl” refers to an aryl group (or rings) that contains at least one heteroatom such as N, O, or S, wherein the nitrogen and sulfur atom(s) are optionally oxidized, and the nitrogen atom(s) is optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described herein.

The term “heteroaryl” may also include multiple condensed ring systems that have at least one such aromatic ring, which multiple condensed ring systems are further described below. The term may also include multiple condensed ring systems (e.g., ring systems comprising 2, 3 or 4 rings) wherein a heteroaryl group, as defined above, can be condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1, 2, 3, 4-tetrahydro-1,8-naphthyridinyl), carbocycles (to form for example 5,6,7, 8-tetrahydroquinolyl) and aryls (to form for example indazolyl) to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g., a nitrogen).

A “heteroarylene,” alone or as part of another substituent, means a divalent radical derived from a heteroaryl.

Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

The terms “halo” or “halogen,” by themselves or as part of another substituent, mean, unless otherwise stated, a fluorine, chlorine, bromine, or iodine atom. Additionally, terms such as “haloalkyl” may include monohaloalkyl and polyhaloalkyl. For example, the term “halo(C₁-C₄)alkyl” may include, but is not limited to, fluoromethyl, difluoromethyl, trifluoromethyl, 2,2,2-trifluoroethyl, 4-chlorobutyl, 3-bromopropyl, 1-fluoro-2-bromoethyl, and the like.

The term “hydroxyl,” as used herein, means —OH.

The term “hydroxyalkyl” as used herein, means an alkyl moiety as defined herein, substituted with one or more, such as one, two or three, hydroxy groups. In certain instances, the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxypropyl.

The term “oxo,” as used herein, means an oxygen that is double bonded to a carbon atom.

A substituent group, as used herein, may be a group selected from the following moieties:
(A) oxo, halogen, —CF₃, —CN, —OH, —OCH₃, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, —OCH₂F, unsubstituted alkyl, unsubstituted cycloalkyl, unsubstituted heterocycloalkyl, unsubstituted aryl, unsubstituted heteroaryl, and
(B) alkyl, cycloalkyl, heterocyclyl, aryl, heteroaryl, substituted with at least one substituent selected from:
   (i) oxo, halogen, —CF₃, —CN, —OH, —OCH₃, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, —OCH₂F, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —OCH₃, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, —OCH₂F, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —OCH₃, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, —OCH₂F, unsubstituted alkyl, unsubstituted heteroalkyl, unsubstituted cycloalkyl, unsubstituted heterocycloalkyl, unsubstituted aryl, unsubstituted heteroaryl.

An “effective amount” when used in connection with a compound is an amount effective for treating or preventing a disease in a subject as described herein.

The term “carrier”, as used in this disclosure, encompasses carriers, excipients, and diluents and may mean a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating may include curing, improving, or at least partially ameliorating the disorder.

The term “prevent” or “preventing” with regard to a subject refers to keeping a disease or disorder from afflicting the subject. Preventing may include prophylactic treatment. For instance, preventing can include administering to the subject a compound disclosed herein before a subject is afflicted with a disease and the administration will keep the subject from being afflicted with the disease.

The term “disorder” is used in this disclosure and means, and is used interchangeably with, the terms disease, condition, or illness, unless otherwise indicated.

The term “administer”, “administering”, or “administration” as used in this disclosure refers to either directly administering a disclosed compound or pharmaceutically acceptable salt or tautomer of the disclosed compound or a composition to a subject, or administering a prodrug derivative or analog of the compound or pharmaceutically acceptable salt or tautomer of the compound or composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

### Compounds

The present disclosure provides a compound having the structure of Formula Ic,

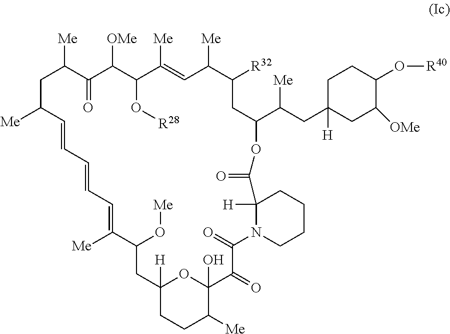

or a pharmaceutically acceptable salt or tautomer thereof, wherein R³², R²⁸, and R⁴⁰ are described as above.

The present disclosure provides a compound having the structure of Formula Ia,

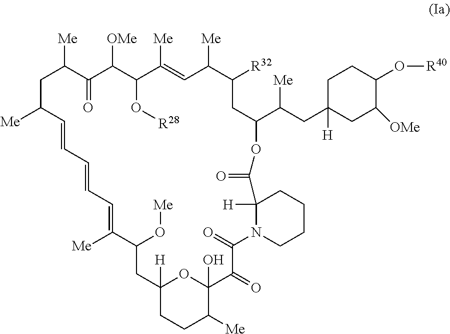

or a pharmaceutically acceptable salt or tautomer thereof, wherein R³², R²⁸, and R⁴⁰ are described as above.

The present disclosure provides a compound having the structure of Formula I,

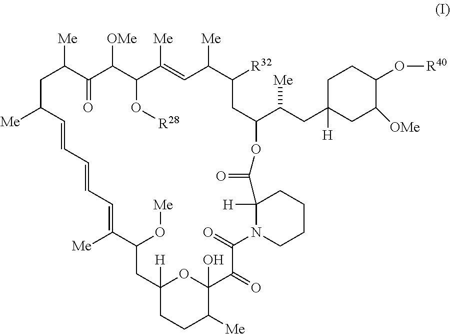

or a pharmaceutically acceptable salt or tautomer thereof, wherein R³², R²⁸, and R⁴⁰ are described as above.

The present disclosure provides a compound having the structure of Formula Ib.

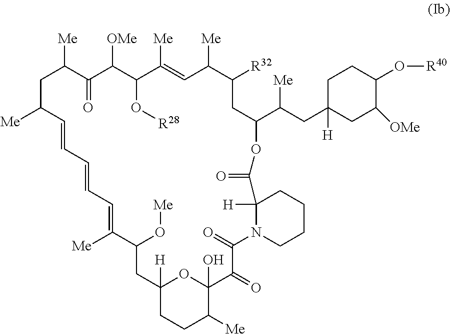

or a pharmaceutically acceptable salt and or tautomer thereof, wherein R³², R²⁸, and R⁴⁰ are described as above for Formula I.

The present disclosure provides a compound having the structure of Formula II,

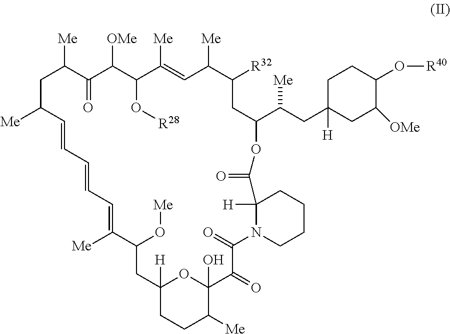

or a pharmaceutically acceptable salt or tautomer thereof, wherein R³², R²⁸, and R⁴⁰ are described as above.

The present disclosure provides a compound having the structure of Formula IIb.

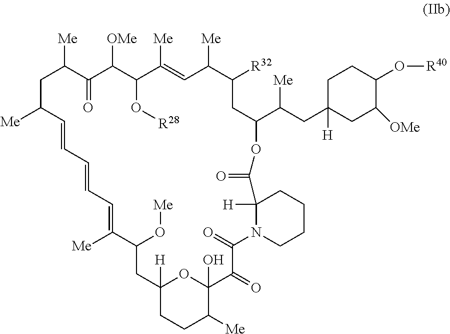

or a pharmaceutically acceptable salt or tautomer thereof, wherein R³², R²⁸, and R⁴ are described as above for Formula II.

In certain embodiments, a compound has the following formula

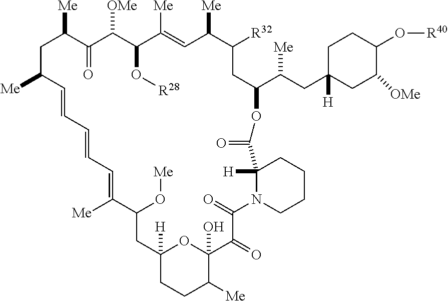

or a pharmaceutically acceptable salt or tautomer thereof.

In certain embodiments, R³² is ═O. In certain embodiments, R³² is —OR³. In certain embodiments, R³² is H. In certain embodiments, R³² is —N₃.

As described above, each R³ is independently H or (C₁-C₆)alkyl. In certain embodiments, R³ is H. In certain embodiments, R³ is (C₁-C₆)alkyl. In certain embodiments, R³ is methyl.

In certain embodiments, R²⁸ is H. In certain embodiments, R²⁸ is (C₁-C₆)alkyl. In certain embodiments, R⁴⁰ is H.

In certain embodiments, a compound is represented by the structure of Formula I-40b:

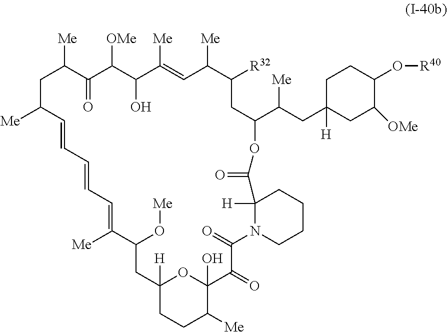

or a pharmaceutically acceptable salt or tautomer thereof, wherein R³² and R⁴⁰ are described as above for Formula Ia, Ic, I, or II.

In certain embodiments, a compound is represented by the structure of Formula I-40:

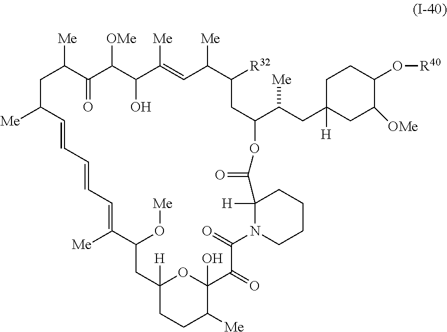

or a pharmaceutically acceptable salt or tautomer thereof, wherein R³² and R⁴⁰ are described as above.

In certain embodiments, R⁴⁰ is —C(═Z¹)—R^{40a}. In certain embodiments, Z¹ is O. In certain embodiments, Z¹ is S.

In certain embodiments, R^{40a} is —O—(C₁-C₆)alkyl or —O—(C₆-C₁₀)aryl; wherein the aryl is unsubstituted or substituted with 1-5 substituents selected from NO₂ and halogen.

In certain embodiments, R^{40a} is -A¹-L¹-A²-B. In certain embodiments, R^{40a} is -A¹-A²-B. In certain embodiments, R^{40a} is -L²-A¹-L¹-A²-L³-B.

In certain embodiments, R^{40a} is -A¹-L¹-A²-B, wherein A¹ and A² are absent. In certain embodiments, R^{40a} is -A¹-L¹-A²-B, wherein A² is absent. In certain embodiments, R^{40a} is -A¹-L¹-A²-B, wherein A¹ is absent. In certain embodiments, R^{40a} is -A¹-L¹-A²-B. In certain embodiments, R^{40a} is -A¹-A²-B. In certain embodiments, R^{40a} is -L²-A¹-L¹-A²-L³-B, wherein L² and A¹ are absent. In certain embodiments, R^{40a} is -L²-A¹-L¹-A²-L³-B, wherein L² is absent. In certain embodiments, R^{40a} is -L²-A¹-L¹-A²-L³-B, wherein L³ is absent.

In certain embodiments, a compounds is represented by the structure of Formula I-28b:

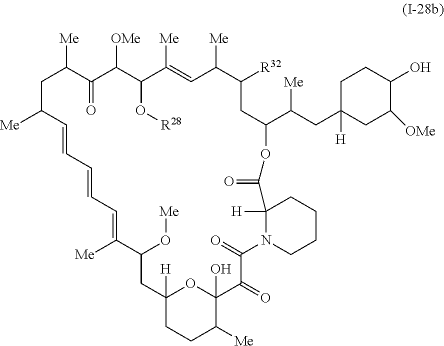

or a pharmaceutically acceptable salt or tautomer thereof, wherein R³² and R²⁸ are described as above for Formula Ia, Ic, I, or II.

In certain embodiments, a compound is represented by the structure of Formula I-28:

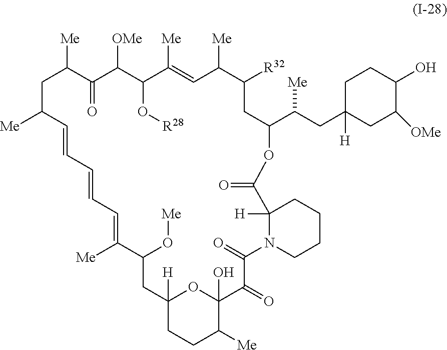

or a pharmaceutically acceptable salt or tautomer thereof, wherein R³² and R²⁸ are described as above.

In certain embodiments, R²⁸ is —C(═Z¹)—R^{28a}. In certain embodiments, Z¹ is O. In certain embodiments, Z¹ is S.

In certain embodiments, R^{28a} is —O—(C₁-C₆)alkyl or —O—(C₆-C₁₀)aryl; wherein the aryl is unsubstituted or substituted with 1-5 substituents selected from NO₂ and halogen.

In certain embodiments, R^{28a} is -A¹-L¹-A²-B. In certain embodiments, R^{28a} is -A¹-A²-B. In certain embodiments, R^{28a} is -L²-A¹-L¹-A²-L³-B.

In certain embodiments, R^{28a} is -A¹-L¹-A²-B, wherein A¹ and A² are absent. In certain embodiments, R^{28a} is -A¹-L¹-A²-B, wherein A² is absent. In certain embodiments, R^{28a} is -A¹-L¹-A²-B, wherein A¹ is absent. In certain embodiments, R^{28a} is -A¹-L¹-A²-B. In certain embodiments, R^{28a} is -A¹-A²-B. In certain embodiments, R^{28a} is -L²-A¹-L¹-A²-L³-B, wherein L² and A¹ are absent. In certain embodiments, R^{28a} is -L²-A¹-L¹-A²-L³-B, wherein L² is absent. In certain embodiments, R^{28a} is -L²-A¹-L¹-A²-L³-B, wherein L³ is absent.

In certain embodiments, the compounds are represented by the structure of Formula I-32b:

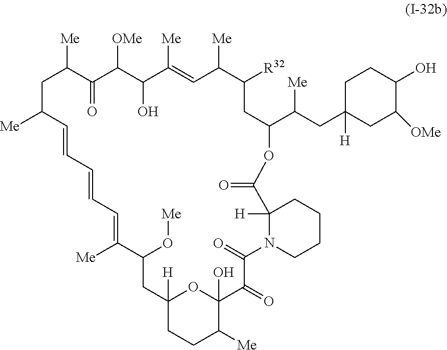

or a pharmaceutically acceptable salt or tautomer thereof, wherein R³² is described as above for Formula Ia, Ic, I, or II.

In certain embodiments, R³² is —O—C(═Z¹)—R^{32a}. In certain embodiments, Z¹ is O. In certain embodiments, Z¹ is S.

In certain embodiments, R^{32a} is —O—(C₁-C₆)alkyl or —O—(C₆-C₁₀)aryl; wherein the aryl is unsubstituted or substituted with 1-5 substituents selected from NO₂ and halogen.

In certain embodiments, R^{32a} is -A¹-L¹-A²-B. In certain embodiments, R^{32a} is -A¹-A²-B. In certain embodiments, R^{32a} is -L²-A¹-L¹-A²-L³-B.

In certain embodiments, R^{32a} is -A¹-L¹-A²-B, wherein A¹ and A² are absent. In certain embodiments, R^{32a} is -A¹-L¹-A²-B, wherein A² is absent. In certain embodiments, R^{32a} is -A¹-L¹-A²-B, wherein A¹ is absent. In certain embodiments, R^{32a} is -A¹-L¹-A²-B. In certain embodiments, R^{32a} is -A¹-A²-B. In certain embodiments, R^{32a} is -L²-A¹-L¹-A²-L³-B, wherein L² and A¹ are absent. In certain embodiments, R^{32a} is -L²-A¹-L¹-A²-L³-B, wherein L² is absent. In certain embodiments, R^{32a} is -L²-A¹-L¹-A²-L³-B, wherein L³ is absent.

As described above, each L¹ is independently selected from

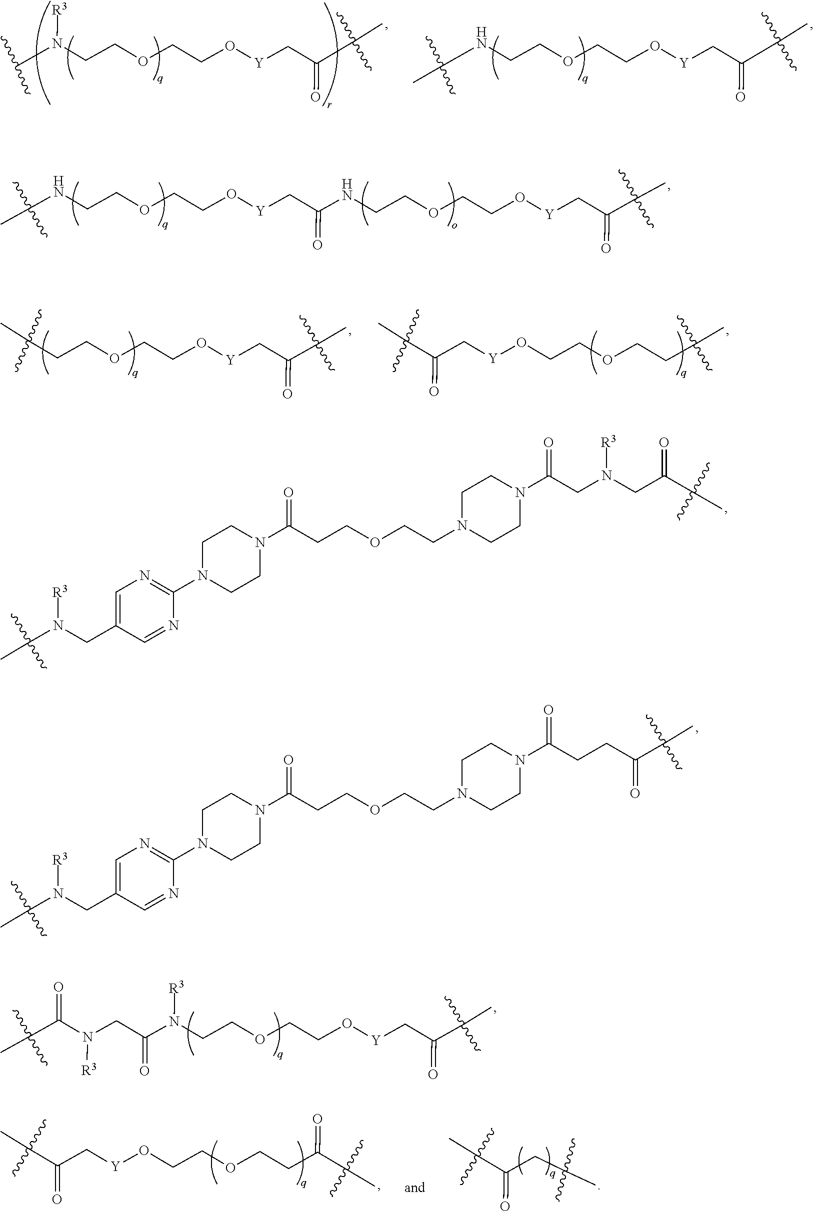

As described above for Formula Ia, each L¹ is independently selected from

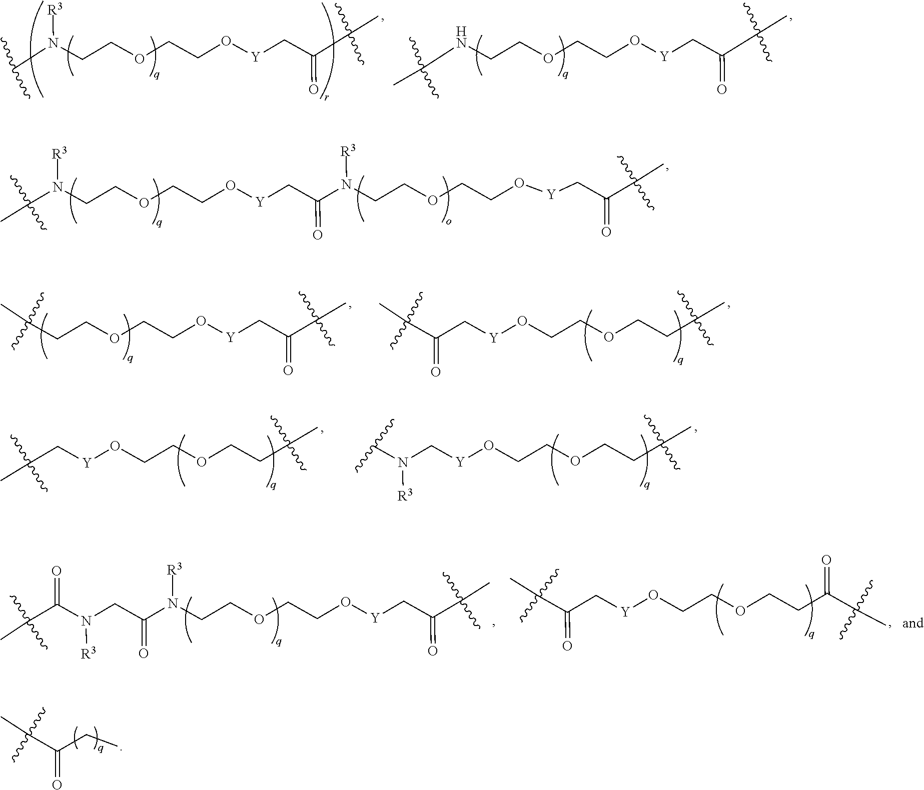

As described above for Formula Ic, each L¹ is independently selected from

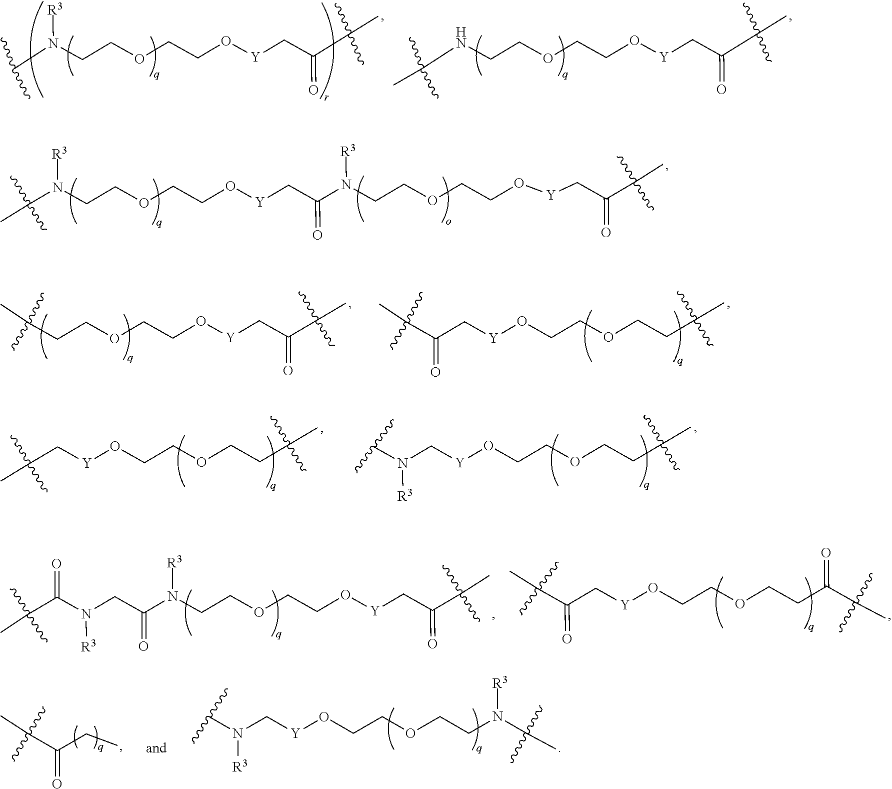

In certain embodiments, L¹

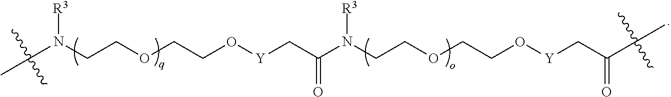

In certain embodiments, L¹ is

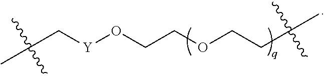

In certain embodiments, L¹ is

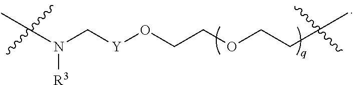

In certain embodiments, L¹ is

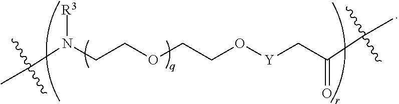

In certain embodiments, L¹ is

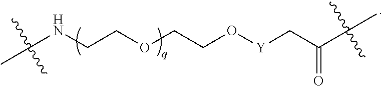

In certain embodiments, L¹ is

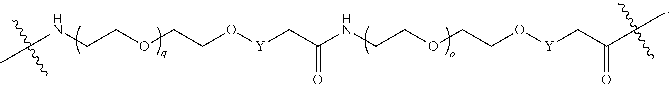

In certain embodiments, L¹ is

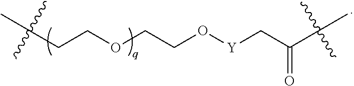

In certain embodiments, L¹ is

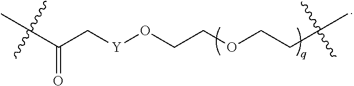

In certain embodiments, L¹ is

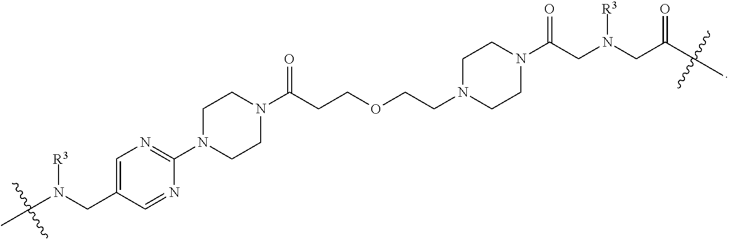

In certain embodiments, L¹ is

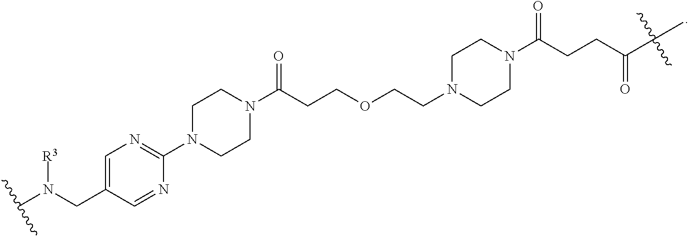

In certain embodiments L¹ is

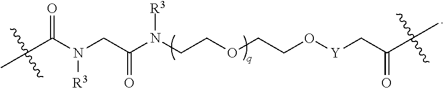

In certain embodiments, L¹ is

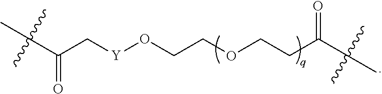

In certain embodiments, L¹ is

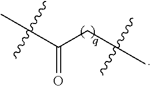 [SMILES: *CC(*)=O]

In certain embodiments, L¹ is

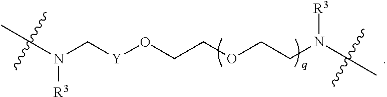

As described above, L² and L³ are independently absent or are independently selected from

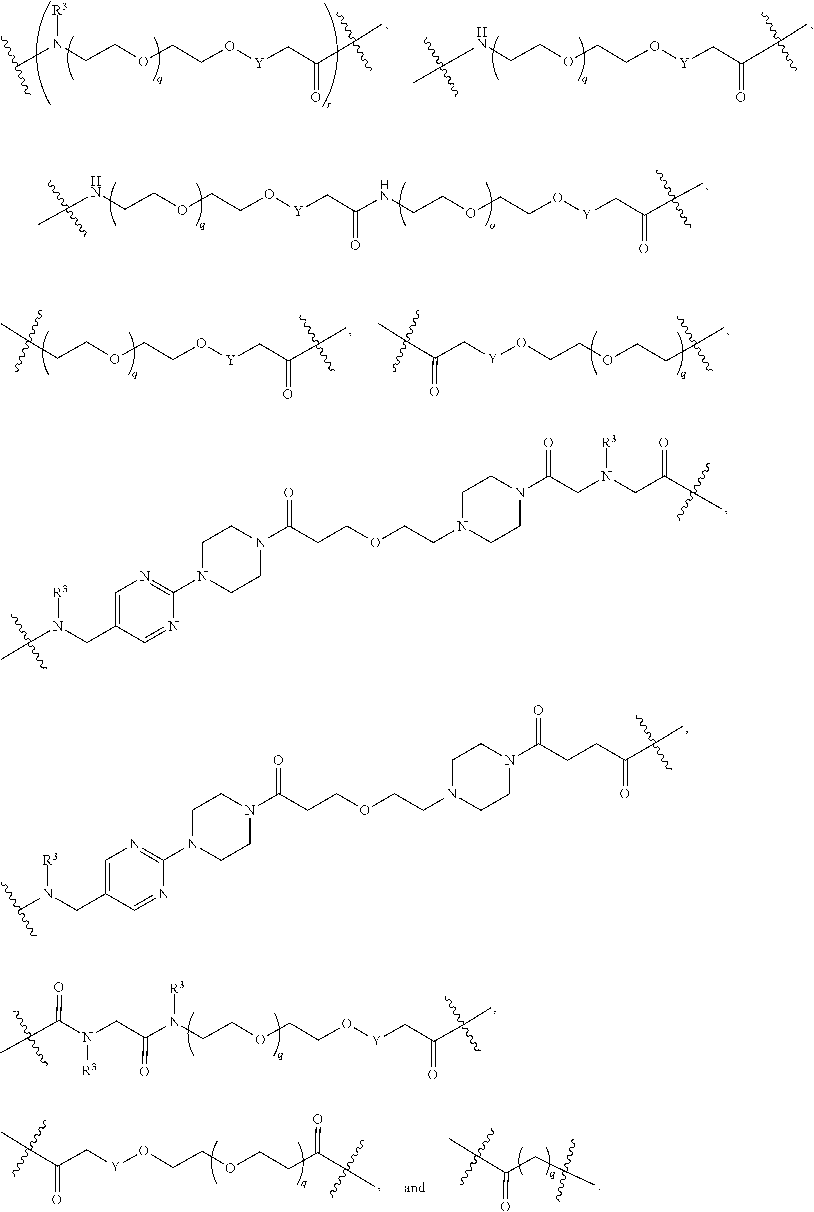

As described above for Formula Ia and Ic, L² and L³ are independently absent or are independently selected from

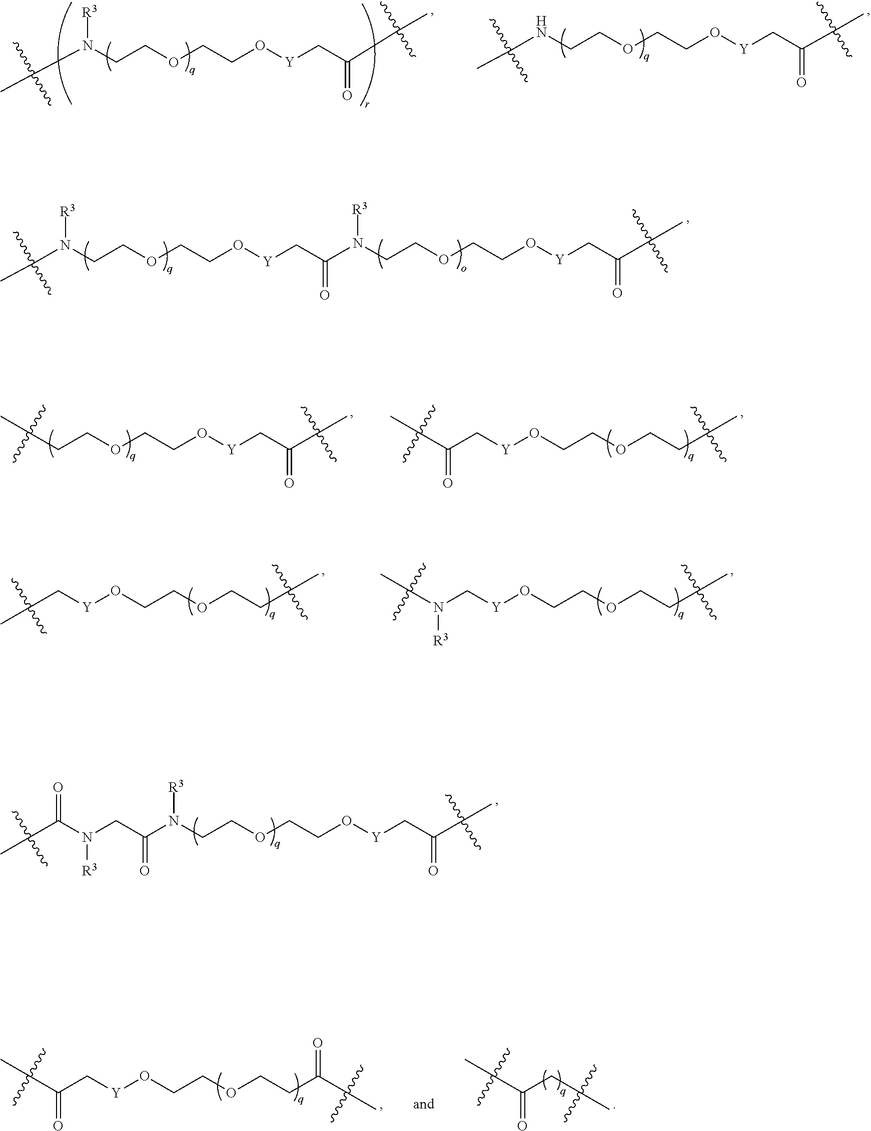

In certain embodiments, L² is absent. In certain embodiments, L² is

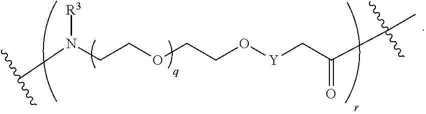

In certain embodiments, L² is

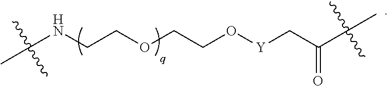

In certain embodiments, L² is

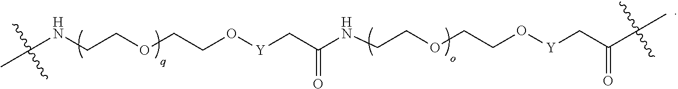

In certain embodiments, L² is

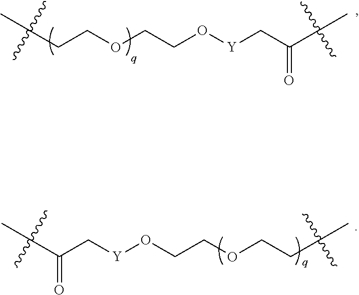

In certain embodiments, L² is

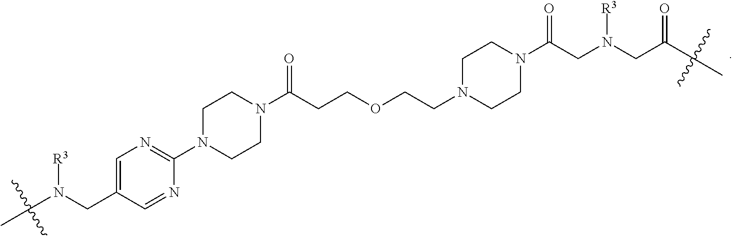

In certain embodiments, L² is

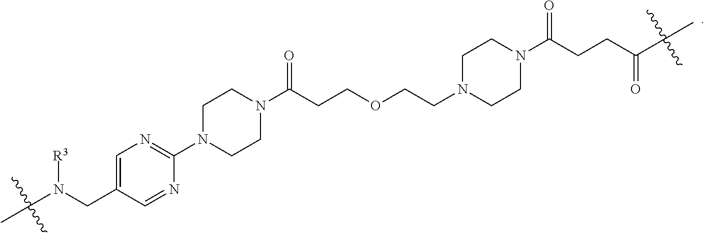

In certain embodiments, L² is

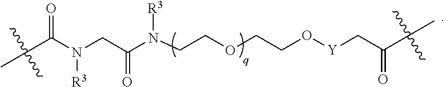

In certain embodiments, L² is

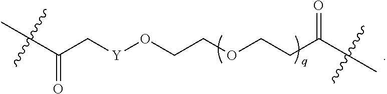

In certain embodiments, L² is

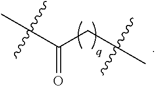

In certain embodiments, L² is

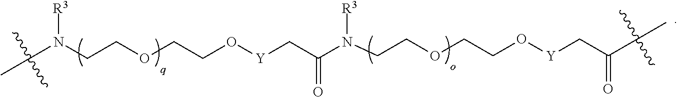

In certain embodiments, L² is

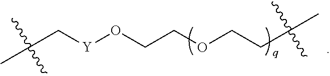

In certain embodiments, L² is

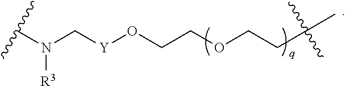

In certain embodiments, L³ is absent. In certain embodiments, L³ is

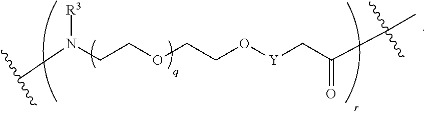

In certain embodiments, L³ is

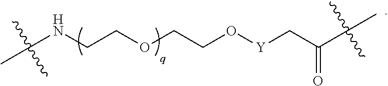

In certain embodiments, L³ is

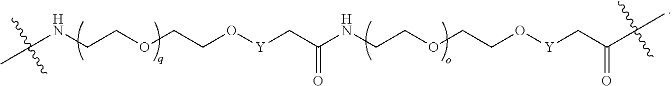

In certain embodiments, L³ is

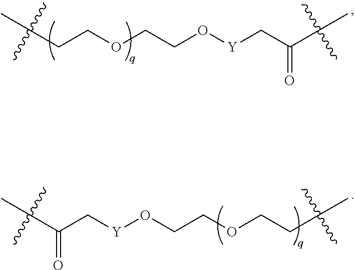

In certain embodiments, L³ is

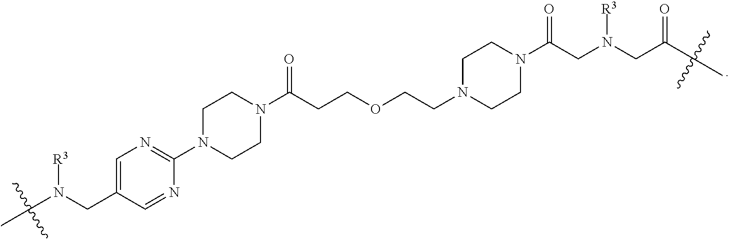

In certain embodiments, L³ is

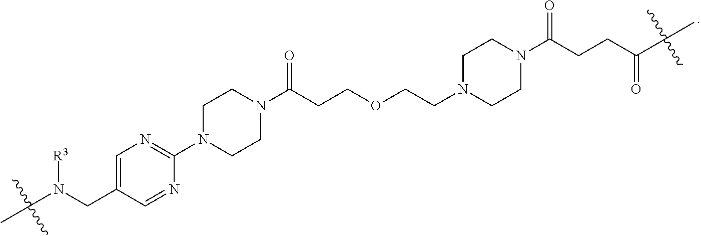

In certain embodiments L³ is

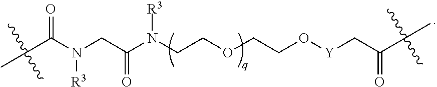

In certain embodiments, L³ is

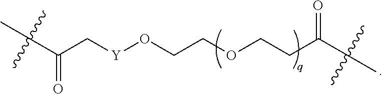

In certain embodiments, L³ is In certain embodiments, L² is

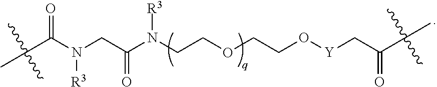

In certain embodiments, L² is

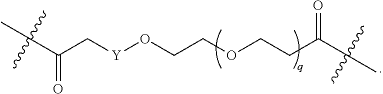

In certain embodiments, L² is

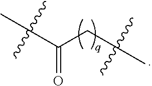

In certain embodiments, L³ is

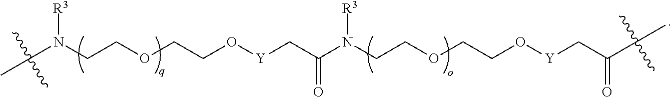

In certain embodiments, L³ is

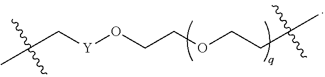

In certain embodiments, L³ is

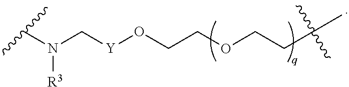

As described above, A¹ and A² are independently absent or are independently selected from

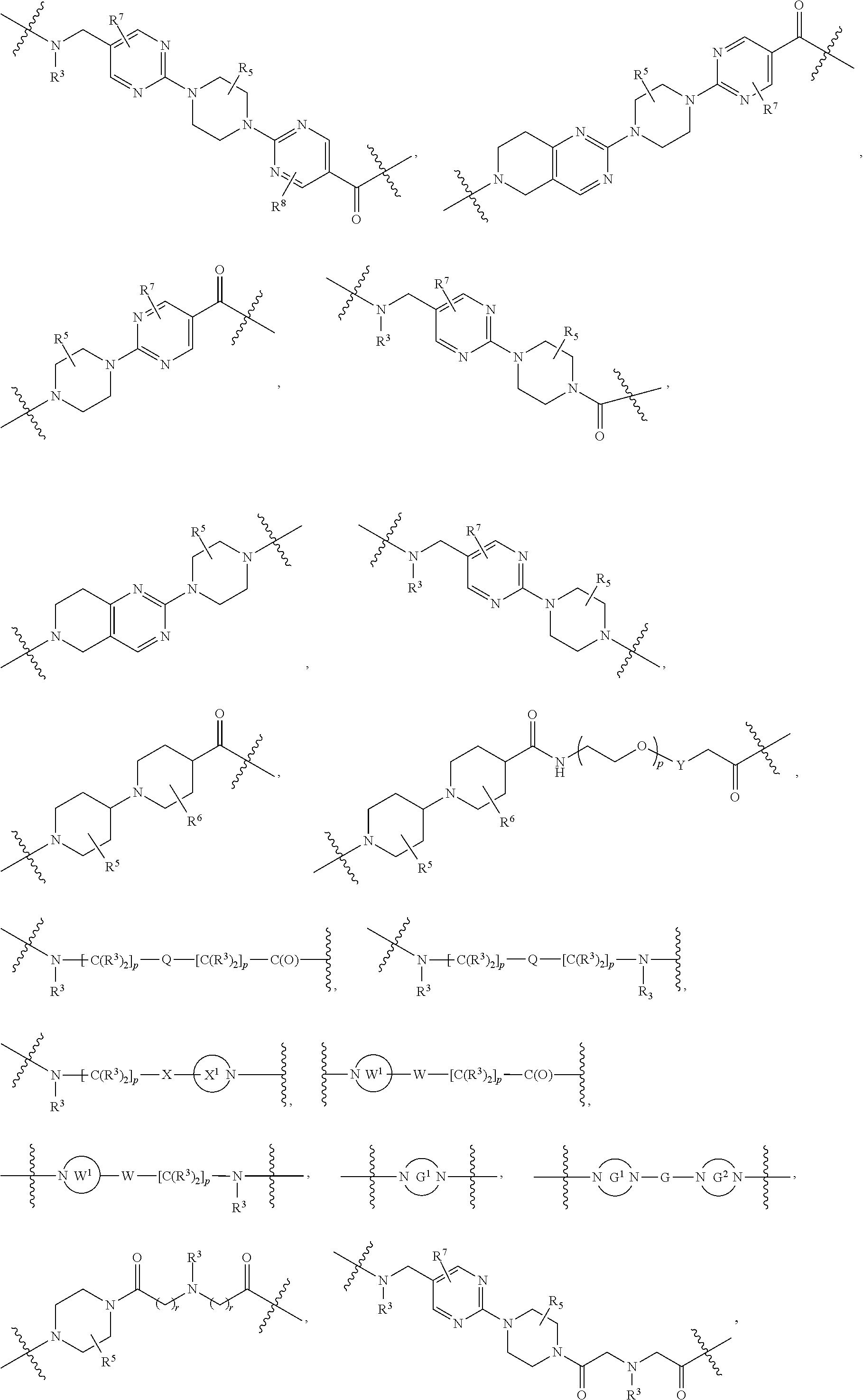

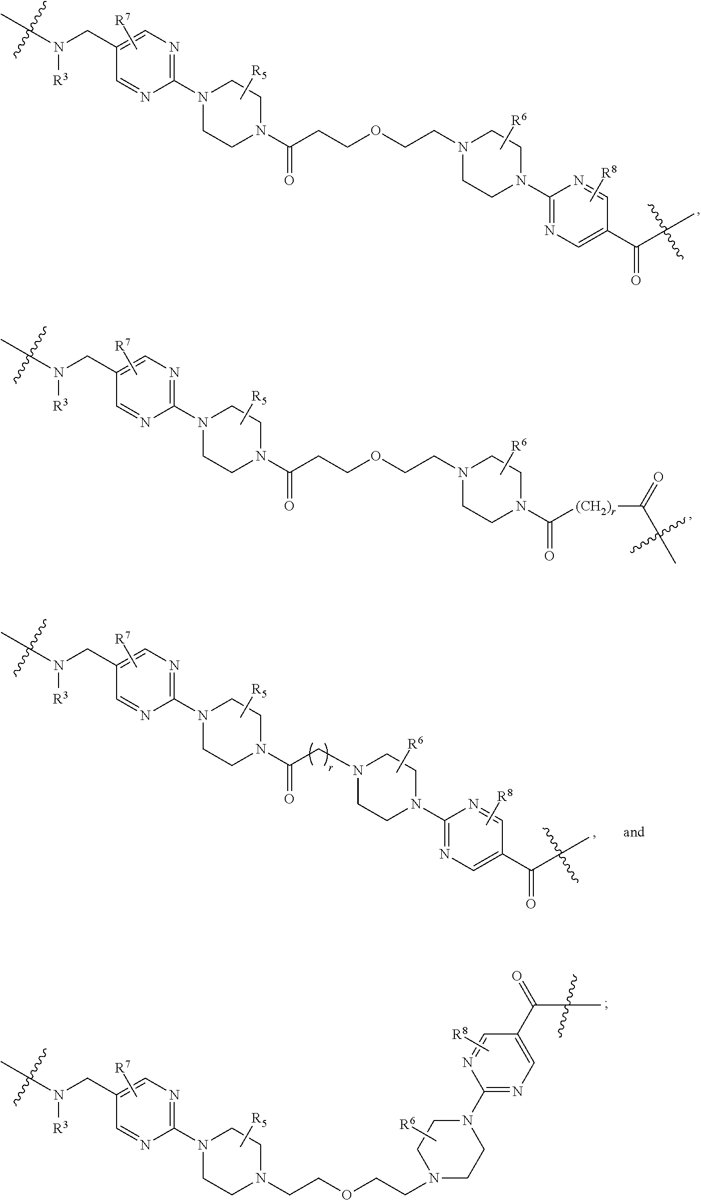

each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ is independently a heteroarylene or heterocyclylene ring;
each G is independently absent or a ring selected from arylene, cycloalkylene, heteroarylene, and heterocyclylene;
each G¹ and G² are independently heteroarylene or heterocyclylene ring.

As described above for Formula Ia, A¹ and A² are independently absent or are independently selected from

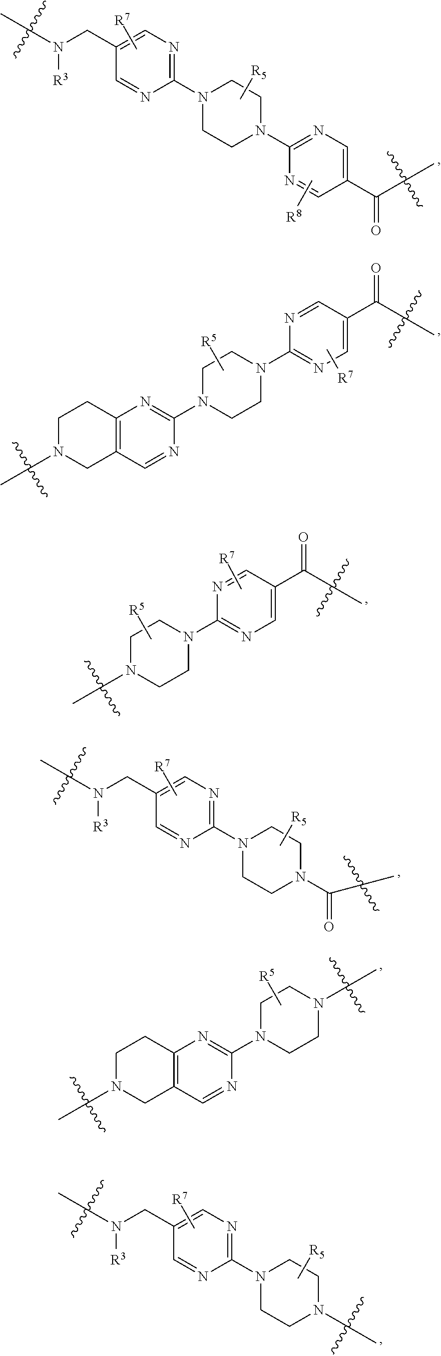

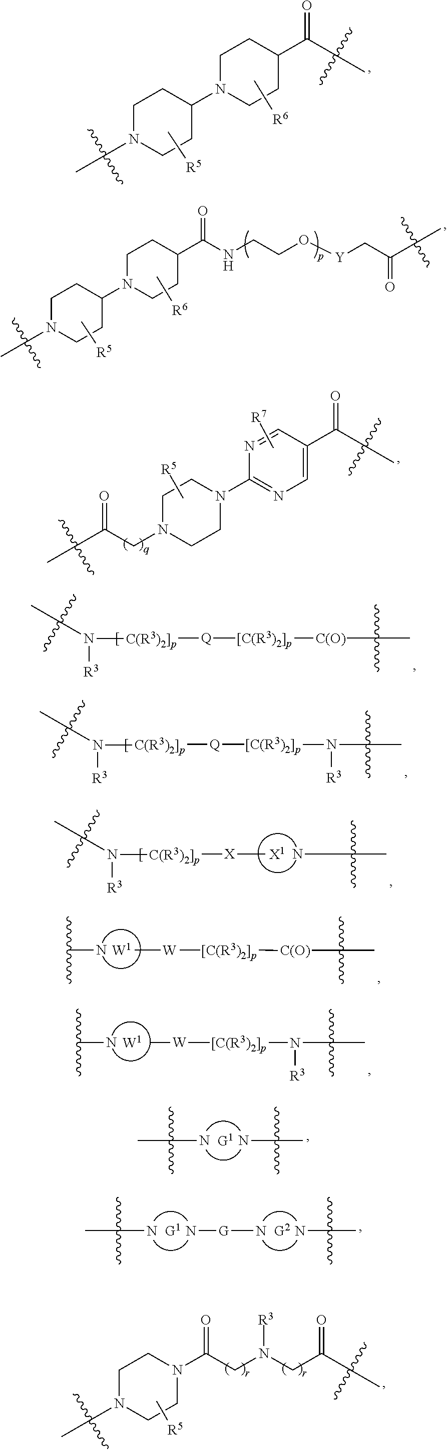

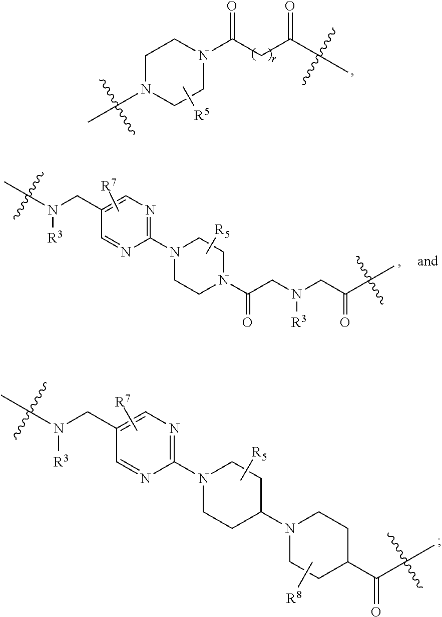

each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ is independently a heteroarylene or heterocyclylene ring;
each G is independently absent or a ring selected from arylene, cycloalkylene, heteroarylene, and heterocyclylene;
each G¹ and G² are independently heteroarylene or heterocyclylene ring.

As described above for Formula Ic, A¹ and A² are independently absent or are independently selected from

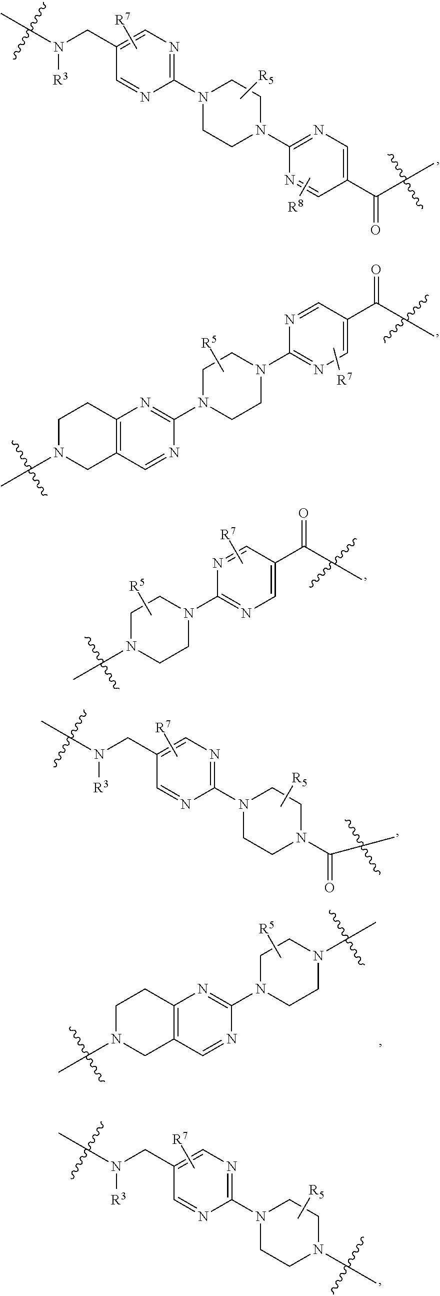

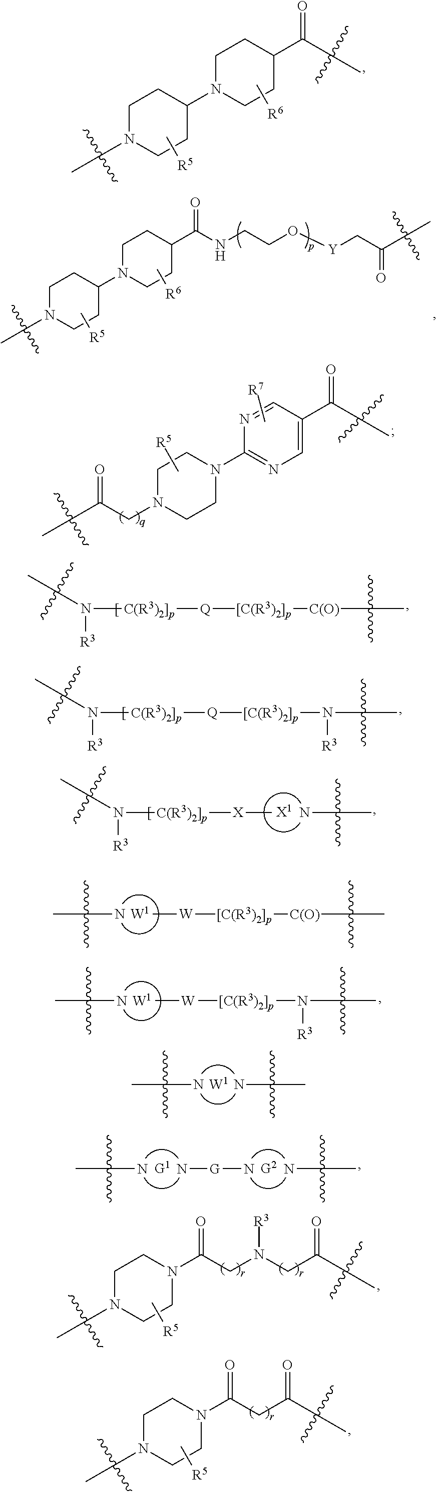

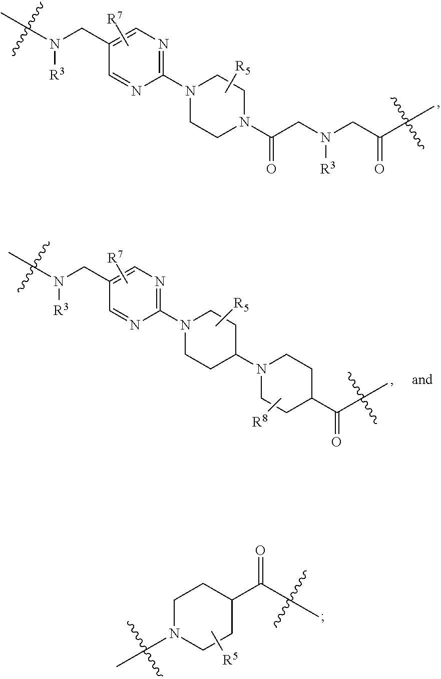

each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ is independently a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;

For Formula I, the bond on the left side of A¹, as drawn, is bound to —C(═Z¹)— or L²; and the bond on the right side of the A² moiety, as drawn, is bound to B or L³. For Formula II, the bond on the left side of A¹, as drawn, is bound to —C(═Z¹)—; and the bond on the right side of the A² moiety, as drawn, is bound to B. For Formula Ia and Ic, the bond on the left side of A¹, as drawn, is bound to —C(═Z¹)— or L²; and wherein the bond on the right side of the A² moiety, as drawn, is bound to B or L³.

In certain embodiments, A¹ is absent. In certain embodiments, A¹ is

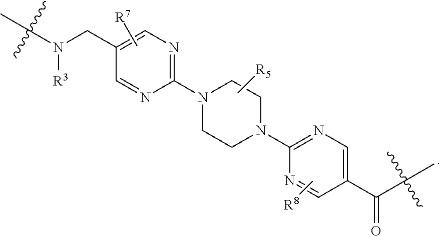

In certain embodiments, A¹ is

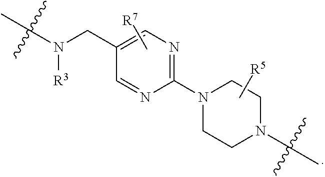

In certain embodiments A¹ is

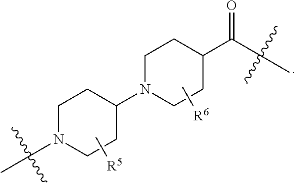

In certain embodiments, A¹ is

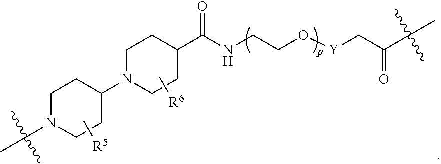

In certain embodiments, A¹ is

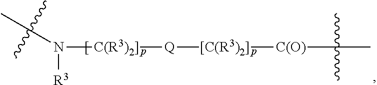

wherein each Q is independently 1 to 3 rings selected from arylene, cycloalkylene, heteroarylene, and heterocyclylene.

In certain embodiments, A¹ is

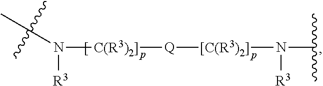

wherein each Q is independently 1 to 3 rings selected from arylene, cycloalkylene, heteroarylene, and heterocyclylene.

In certain embodiments, A¹ is

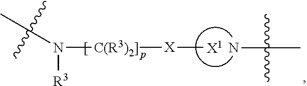

wherein each X is independently absent or 1 to 2 rings selected from arylene, cycloalkylene, heteroarylene, and heterocyclylene; and each X¹ is a heteroarylene or heterocyclylene ring.

In certain embodiments, A¹ is

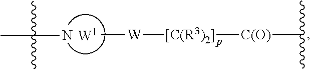

wherein each W is independently absent or 1 to 2 rings selected from arylene, cycloalkylene, heteroarylene, and heterocyclylene; and each W¹ is a heteroarylene or heterocyclylene ring.

In certain embodiments, A¹ is

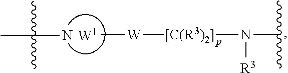

wherein each W is independently absent or 1 to 2 rings selected from arylene, cycloalkylene, heteroarylene, and heterocyclylene; and each W¹ is a heteroarylene or heterocyclylene ring.

In certain embodiments, A¹ is

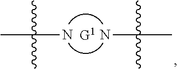

wherein each G is independently absent or a ring selected from arylene, cycloalkylene, heteroarylene, and heterocyclylene.

In certain embodiments, A¹ is

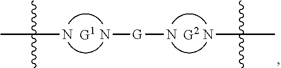

wherein each G is independently absent or a ring selected from arylene, cycloalkylene, heteroarylene, and heterocyclylene; and each G¹ and G² are independently heteroarylene or heterocyclylene ring.

In certain embodiments, A¹ is

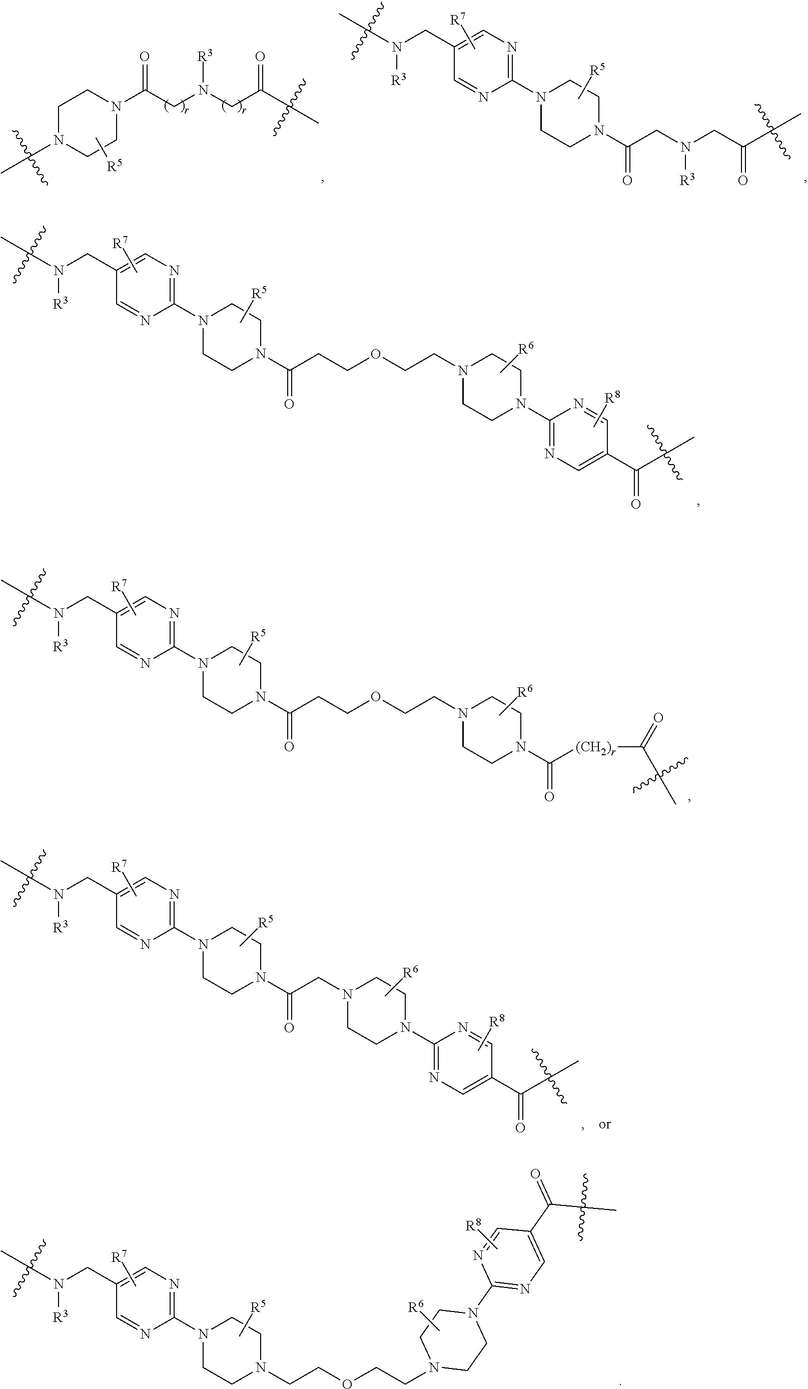

In certain embodiments, A¹ is

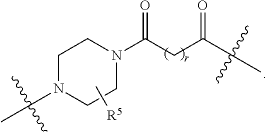

In certain embodiments, A¹ is

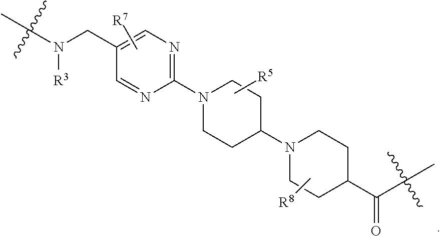

In certain embodiments, A¹ is

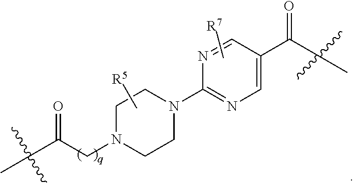

In certain embodiments, A¹ is

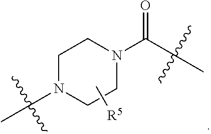

In certain embodiments, A² is absent. In certain embodiments, A² is

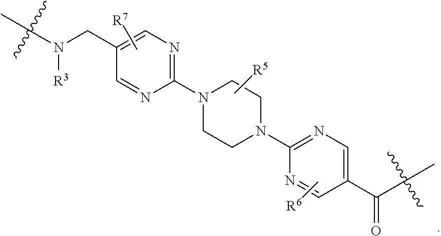

In certain embodiments, A² is

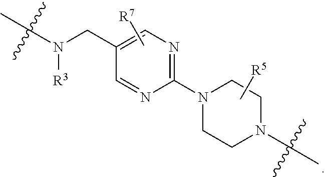

In certain embodiments A² is

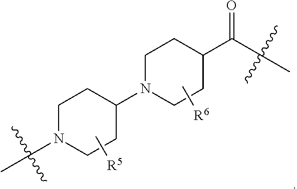

In certain embodiments, A² is

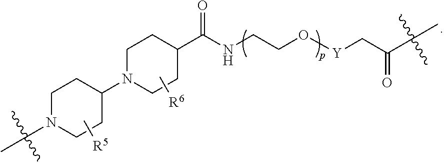

In certain embodiments, A² is

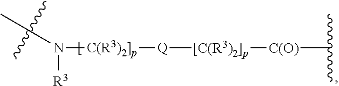

wherein each Q is independently 1 to 3 rings selected from arylene, cycloalkylene, heteroarylene, and heterocyclylene.

In certain embodiments, A² is

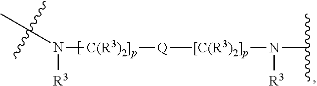

wherein each Q is independently 1 to 3 rings selected from arylene, cycloalkylene, heteroarylene, and heterocyclylene.

In certain embodiments, A² is

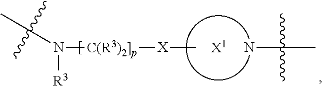

wherein each X is independently absent or 1 to 2 rings selected from arylene, cycloalkylene, heteroarylene, and heterocyclylene; and each X¹ is independently a heteroarylene or heterocyclylene ring.

In certain embodiments, A² is

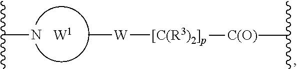

wherein each W is independently absent or 1 to 2 rings selected from arylene, cycloalkylene, heteroarylene, and heterocyclylene; and each W¹ is independently a heteroarylene or heterocyclylene ring.

In certain embodiments, A² is

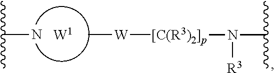

wherein each W is independently absent or 1 to 2 rings selected from arylene, cycloalkylene, heteroarylene, and heterocyclylene; and each W¹ is independently a heteroarylene or heterocyclylene ring.

In certain embodiments, A² is

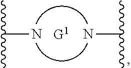

wherein each G is independently absent or a ring selected from arylene, cycloalkylene, heteroarylene, and heterocyclylene.

In certain embodiments, A² is

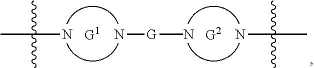

wherein each G is independently absent or a ring selected from arylene, cycloalkylene, heteroarylene, and heterocyclylene; and each G¹ and G² are independently a heteroarylene or heterocyclylene ring.

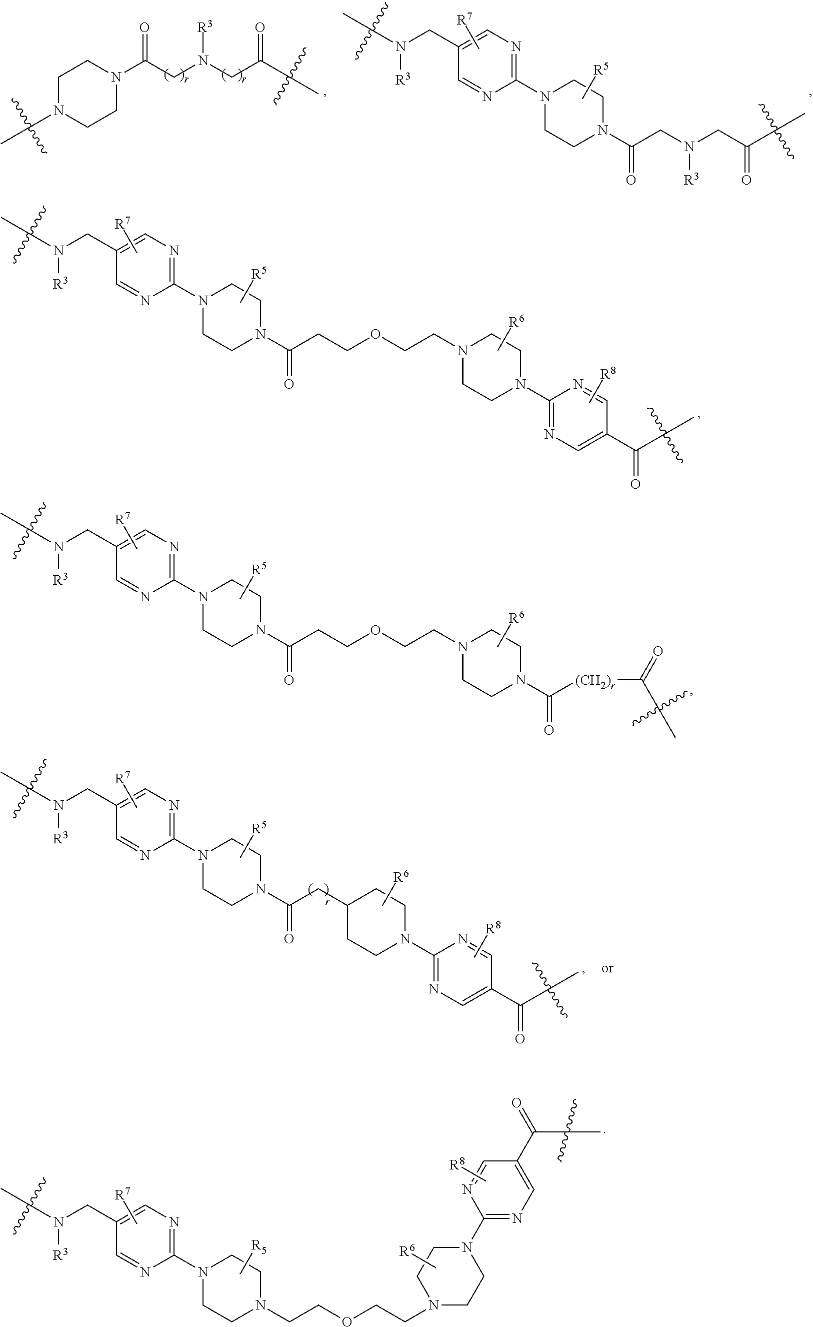

In certain embodiments, A² is

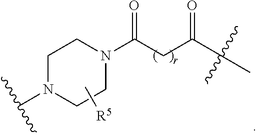

In certain embodiments, A² is

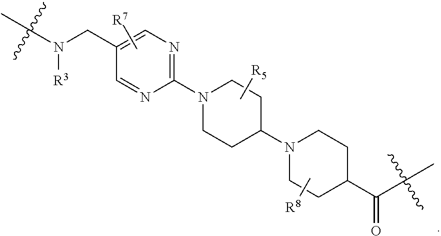

In certain embodiments, A² is

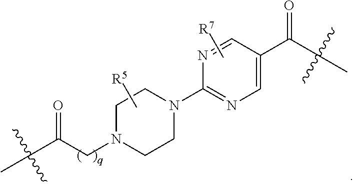

In certain embodiments, A² is

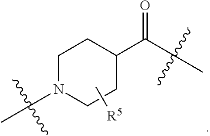

As described above, each B is independently selected from

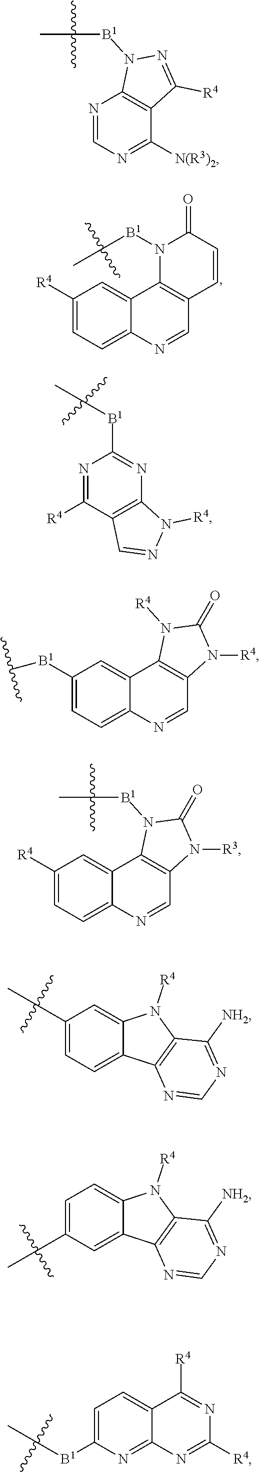

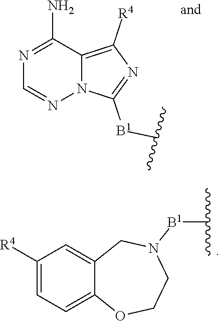

In certain embodiments, B is

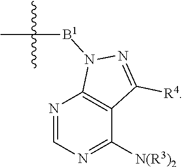

In certain embodiments, B is

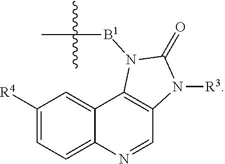

In certain embodiments, B is

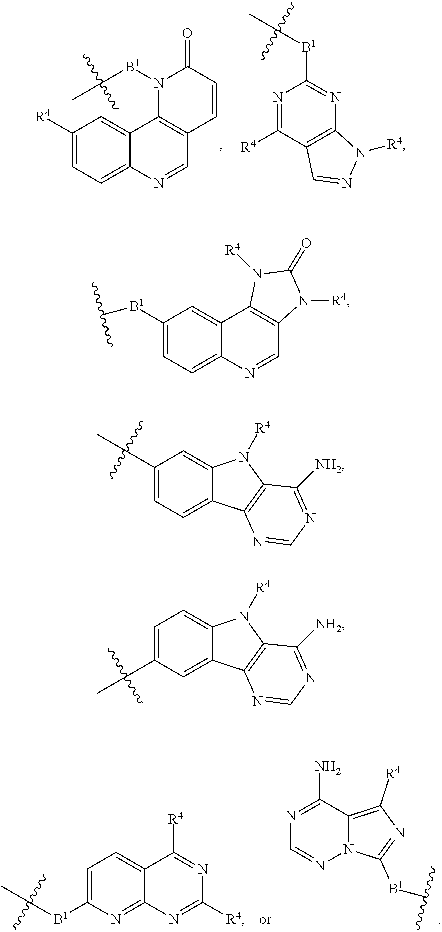

In certain embodiments, B is

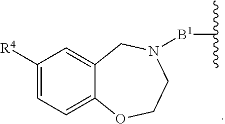

As described above, each B¹ is independently selected from

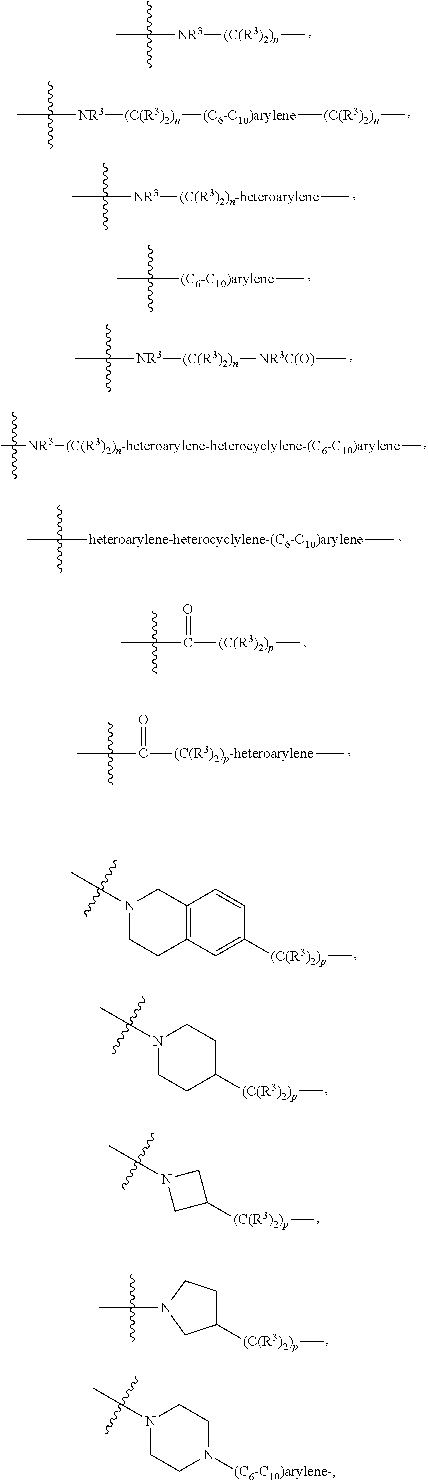

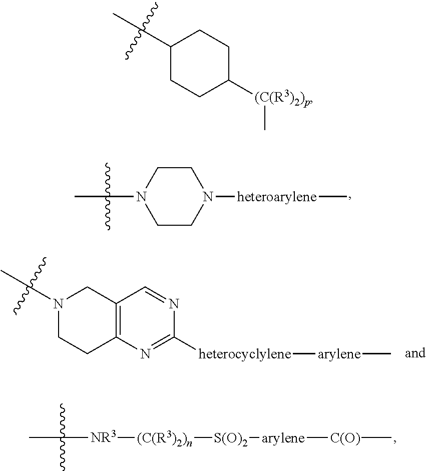

wherein the

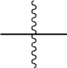

bond on the left side of B¹, as drawn, is bound to A² or L¹; and wherein the heteroarylene, heterocyclylene, and arylene are each independently optionally substituted with alkyl, hydroxyalkyl, haloalkyl, alkoxy, halogen, or hydroxyl.

As described above for Formula Ic, each B¹ is independently selected from

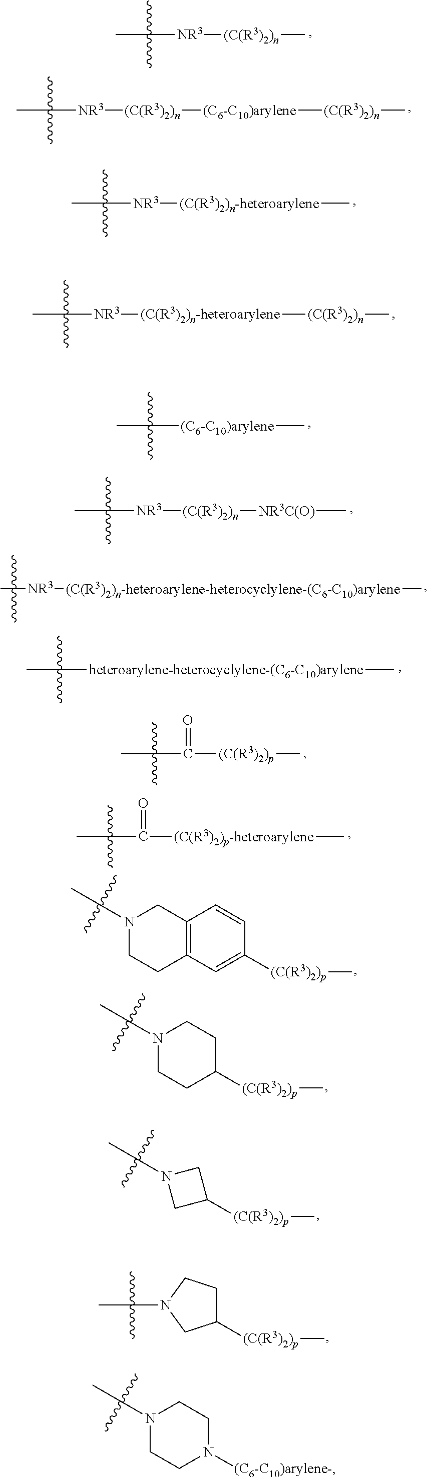

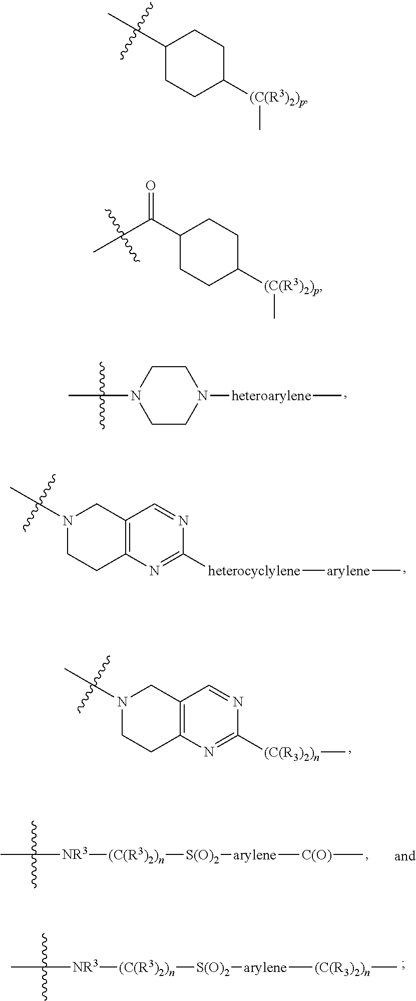

wherein the

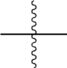

bond on the left side of B¹, as drawn, is bound to A², L³, or L¹; and wherein the heteroarylene, heterocyclylene, and arylene are each independently optionally substituted with alkyl, hydroxyalkyl, haloalkyl, alkoxy, halogen, or hydroxyl.

In certain embodiments, B¹ is

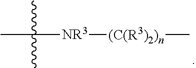

In certain embodiments, B¹ is

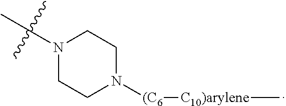

In certain embodiments, B¹ is

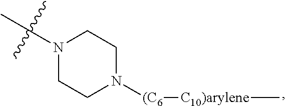

wherein arylene is optionally substituted with haloalkyl.

In certain embodiments, B¹ is

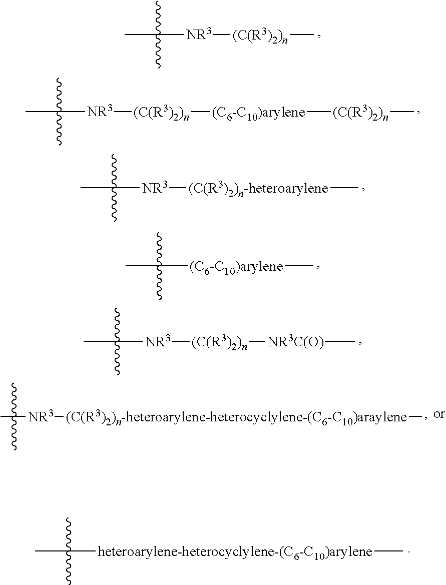

In certain embodiments, B¹ is

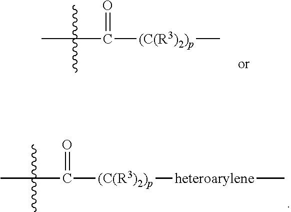

In certain embodiments, B¹ is

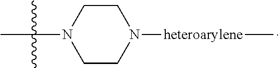

In certain embodiments, B¹ is

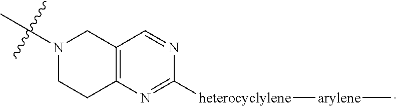

In certain embodiments, B¹ is

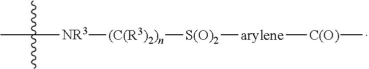

In certain embodiments, B¹ is

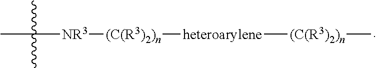

In certain embodiments, B¹ is

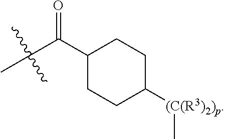

In certain embodiments, B¹ is

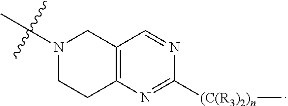

In certain embodiments, B¹ is

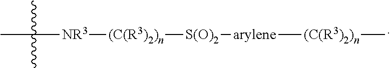

In certain embodiments, in B¹, the heteroaryl, heterocyclyl, and arylene are optionally substituted with alkyl, hydroxyalkyl, haloalkyl, alkoxy, halogen, or hydroxyl.

In certain embodiments, R³ is H. In certain embodiments, R³ is (C₁-C₆)alkyl.

In certain embodiments, R⁴ is H. In certain embodiments, R⁴ is (C₁-C₆)alkyl. In certain embodiments, R⁴ is halogen. In certain embodiments, R⁴ is 5-12 membered heteroaryl, 5-12 membered heterocyclyl, or (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl. In certain embodiments, R⁴ is —C(O)NR³-heterocyclyl. In certain embodiments, R⁴ is 5-12 membered heteroaryl, optionally substituted with —N(R³)₂ or —OR³.

As described above, each R⁵ is independently H, (C₁-C₆)alkyl, —C(O)OR³, or —N(R³)₂, wherein the alkyl of (C₁-C₆)alkyl is optionally substituted with —N(R³)₂ or —OR³. In certain embodiments, R⁵ is H. In certain embodiments, R⁵ is (C₁-C₆)alkyl, wherein the alkyl is optionally substituted with —N(R³)₂ or —OR³. In certain embodiments, R⁵ is —C(O)OR³. In certain embodiments, R⁵ is —N(R³)₂.

As described above, each R⁶ is independently H, (C₁-C₆)alkyl, —C(O)OR³, or —N(R³)₂, wherein the alkyl of (C₁-C₆)alkyl is optionally substituted with —N(R³)₂ or —OR³. In certain embodiments, R⁶ is H. In certain embodiments, R⁶ is (C₁-C₆)alkyl, wherein the alkyl is optionally substituted with —N(R³)₂ or —OR³. In certain embodiments, R⁶ is —C(O)OR³. In certain embodiments, R⁶ is —N(R³)₂.

As described above, each R⁷ is independently H, (C₁-C₆)alkyl, —C(O)OR³, or —N(R³)₂, wherein the alkyl of (C₁-C₆)alkyl is optionally substituted with —N(R³)₂ or —OR³. In certain embodiments, R⁷ is H. In certain embodiments, R⁷ is (C₁-C₆)alkyl, wherein the alkyl is optionally substituted with —N(R³)₂ or —OR³. In certain embodiments, R⁷ is —C(O)OR³. In certain embodiments, R⁷ is —N(R³)₂.

As described above, each R⁸ is independently H, (C₁-C₆)alkyl, —C(O)OR³, or —N(R³)₂, wherein the alkyl of (C₁-C₆)alkyl is optionally substituted with —N(R³)₂ or —OR³. In certain embodiments, R⁸ is H. In certain embodiments, R⁸ is (C₁-C₆)alkyl, wherein the alkyl is optionally substituted with —N(R³)₂ or —OR³. In certain embodiments, R⁸ is —C(O)OR³. In certain embodiments, R⁸ is —N(R³)₂.

As described above, each Y is independently C(R³)₂ or a bond. In certain embodiments, Y is C(R³)₂. In certain embodiments, Y is CH₂. In certain embodiments, Y is a bond.

In certain embodiments, n is 1, 2, 3, 4, 5, 6, 7, or 8, or any range derivable therein. In certain embodiments, n is 1, 2, 3, or 4. In certain embodiments, n is 5, 6, 7, or 8. In certain embodiments, n is 9, 10, 11, or 12.

In certain embodiments, o is an integer from zero to 10, or any range derivable therein. In certain embodiments, o is 0, 1, 2, 3, 4, or 5. In certain embodiments, o is 6, 7, 8, 9, or 10. In certain embodiments, o is one to 7. In certain embodiments, o is one to 8. In certain embodiments, o is one to 9. In certain embodiments, o is 3 to 8.

In certain embodiments, o is an integer from zero to 30, or any range derivable therein. In certain embodiments, o is an integer from zero to 30, 29, 28, 27, or 26. In certain embodiments, o is an integer from zero to 25, 24, 23, 22, or 21. In certain embodiments, o is an integer from zero to 20, 19, 18, 17, or 16. In certain embodiments, o is an integer from zero to 15, 14, 13, 12, or 11.

In certain embodiments, p is 0, 1, 2, 3, 4, 5, or 6, or any range derivable therein. In certain embodiments, p is 7, 8, 9, 10, 11, or 12. In certain embodiments, p is 0, 1, 2, or 3. In certain embodiments, p is 4, 5, or 6.

In certain embodiments, q is an integer from zero to 10, or any range derivable therein. In certain embodiments, q is 0, 1, 2, 3, 4, or 5. In certain embodiments, q is 6, 7, 8, 9, or 10. In certain embodiments, q is one to 7. In certain embodiments, q is one to 8. In certain embodiments, q is one to 9. In certain embodiments, q is 3 to 8.

In certain embodiments, q is an integer from zero to 30, or any range derivable therein. In certain embodiments, q is an integer from zero to 30, 29, 28, 27, or 26. In certain embodiments, q is an integer from zero to 25, 24, 23, 22, or 21. In certain embodiments, q is an integer from zero to 20, 19, 18, 17, or 16. In certain embodiments, q is an integer from zero to 15, 14, 13, 12, or 11.

As described above, r is an integer from one to 6. In certain embodiments, r is one. In certain embodiments, r is 2. In certain embodiments, r is 3. In certain embodiments, r is 4. In certain embodiments, r is 5. In certain embodiments, r is 6.

As described above, when R²⁸ and R⁴⁰ are H, then R³² is not ═O. In certain embodiments, the compound is not rapamycin, as shown below:

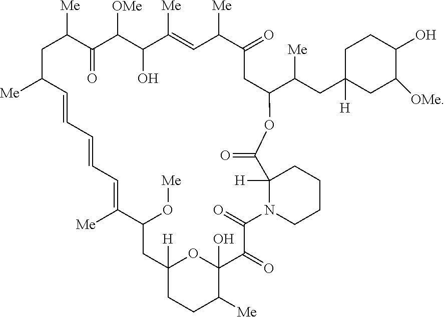 [SMILES: COC1CC2CCC(C)C(O)(O2)C(=O)C(=O)N2CCCCC2C(=O)OC(C(C)CC2CCC(O)C(OC)C2)CC(=O)C(C)/C=C(\C)C(O)C(OC)C(=O)C(C)CC(C)/C=C/C=C/C=C/1C]

In certain embodiments, in Formula Ia or Ic, R³² is —O—C(═Z)—R^{32a}. In certain embodiments, R³² is —O—C(═Z¹)—R^{32a}; wherein R^{32a} is -A¹-L¹-A²-B; -A¹-A²-B; or -L²-A¹-L¹-A²-L³-B. In certain embodiments, in Formula Ia or Ic, R²⁸ is —C(═Z¹)—R^{28a}. In certain embodiments, R²⁸ is —C(═Z¹)—R^{28a}; wherein R^{28a} is -A¹-L¹-A²-B; -A¹-A²-B; or -L²-A¹-L¹-A²-L³-B. In certain embodiments, in Formula Ia or Ic, R⁴⁰ is —C(═Z¹)—R^{40a}. In certain embodiments, R⁴⁰ is —C(═Z¹)—R^{40a}. wherein R^{40a} is -A¹-L¹-A²-B; -A¹-A²-B; or -L²-A¹-L¹-A²-L³-B.

The present disclosure provides a compound of Formula Ia or Ic, or a pharmaceutically acceptable salt or tautomer thereof, having one, two, or three of the following features:
a) R³² is —O—C(═Z¹)—R^{32a};
b) R²⁸ is —C(═Z¹)—R^{28a};
c) R⁴⁰ is —C(═Z¹)—R^{40a}.

The present disclosure provides a compound of Formula Ia or Ic, or a pharmaceutically acceptable salt or tautomer thereof, having one, two, or three of the following features:
a) R³² is —O—C(═Z¹)—R^{32a}; wherein R^{32a} is -A¹-L¹-A²-B; -A¹-A²-B; or -L²-A¹-L¹-A²-L³-B³;
b) R²⁸ is —C(═Z¹)—R^{28a}; wherein R^{28a} is -A¹-L¹-A²-B; -A¹-A²-B; or -L²-A¹-L¹-A²-L³-B³;
c) R⁴⁰ is —C(═Z¹)—R^{40a}. wherein R^{40a} is -A¹-L¹-A²-B; -A¹-A²-B; or -L²-A¹-L¹-A²-L³-B.

The present disclosure provides a compound of Formula Ia or Ic, or a pharmaceutically acceptable salt or tautomer thereof, having one, two, or three of the following features:
a) R⁴⁰ is —C(═Z¹)—R^{40a};
b) R^{40a} is -A¹-L¹-A²-B; -A¹-A²-B; -L²-A¹-L¹-A²-L³-B;
c) R³² is —OR³, such as —OH.

The present disclosure provides a compound of Formula Ia or Ic, or a pharmaceutically acceptable salt or tautomer thereof, having one, two, three, or four of the following features:
a) one of R^{28a}, R^{32a}, and R^{40a} is -A¹-L¹-A²-B;
b) A¹ is absent;
c) A² is absent;
d) L¹ is

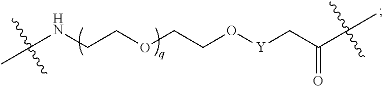

e) B is

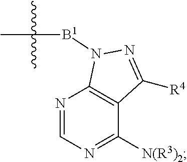

f) B¹ is

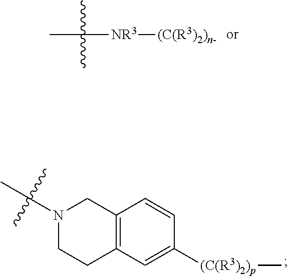

g) R⁴ is 5-12 membered heteroaryl, optionally substituted with —N(R³)₂ or —OR³.

The present disclosure provides a compound of formula:

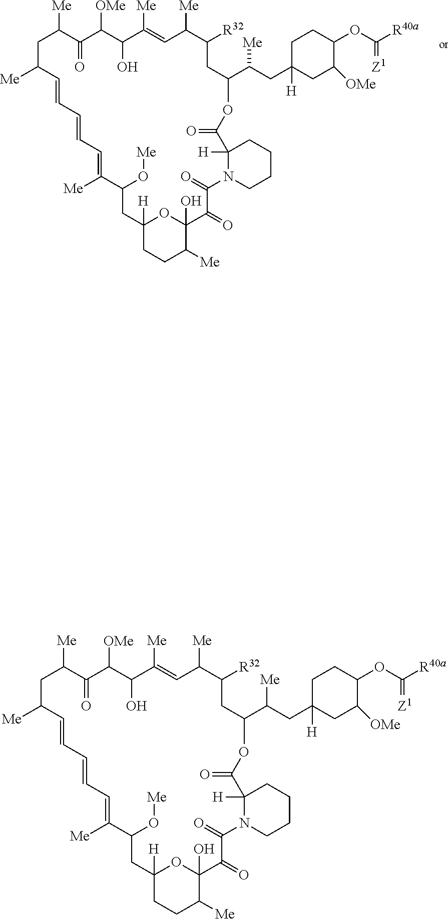

or a pharmaceutically acceptable salt or tautomer thereof, having one, two, three, or four of the following features:
a) Z¹ is O;
b) A¹ is absent;
c) L¹ is

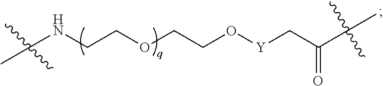

d) B is

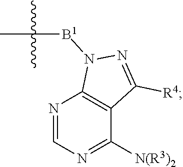

e) B¹ is

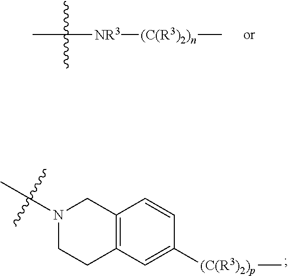

f) R⁴ is 5-12 membered heteroaryl, optionally substituted with —N(R³)₂ or —OR³; and
g) R³² is ═O.
   In the above, R^{40a} can be -A¹-L¹-A²-B; -A¹-A²-B; or -L²-A¹-L¹-A²-L³-B.

The present disclosure provides a compound of formula:

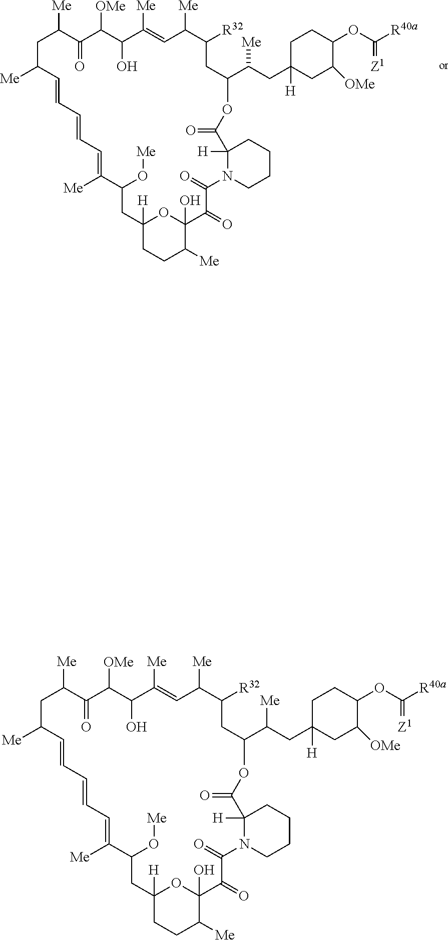

or a pharmaceutically acceptable salt or tautomer thereof, having one, two, three, or four of the following features:
a) Z¹ is O;
b) A¹ is absent;
c) L¹ is

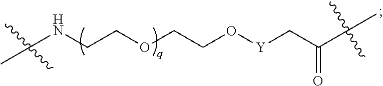

d) B is

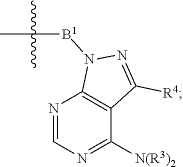

e) B¹ is

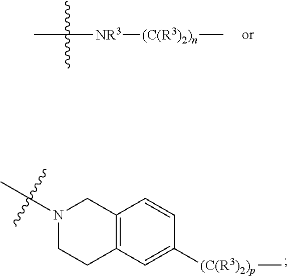

f) R⁴ is 5-12 membered heteroaryl, optionally substituted with —N(R³)₂ or —OR³; and
g) R³² is —OH.
   In the above, R^{40a} can be -A¹-L¹-A²-B; -A¹-A²-B; or -L²-A¹-L¹-A²-L³-B.

The present disclosure provides a compound of formula:

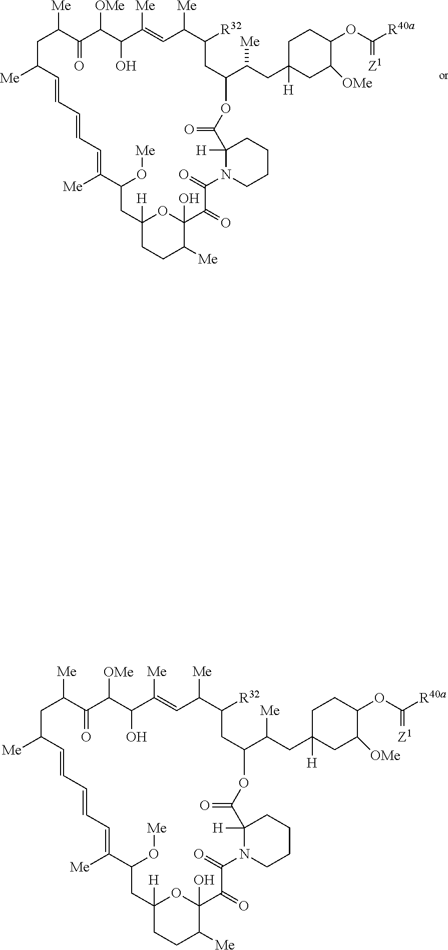

or a pharmaceutically acceptable salt or tautomer thereof, having one, two, three, or four of the following features:
a) Z¹ is O;
b) A¹ is

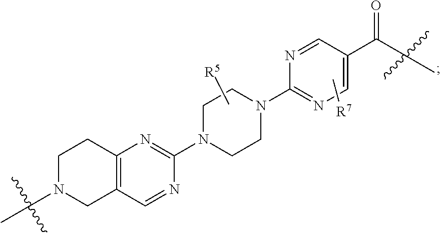

c) L¹ is

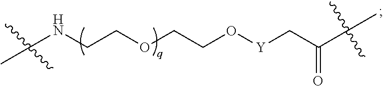

d) B is

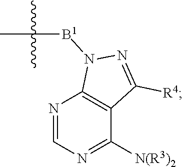

e) B¹ is

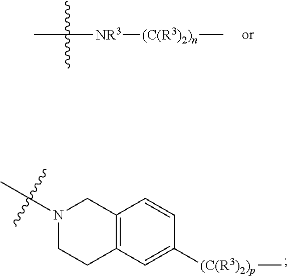

f) R⁴ is 5-12 membered heteroaryl, optionally substituted with —N(R³)₂ or —OR³; and
g) R³² is ═O.
   In the above, R^{40a} can be -A¹-L¹-A²-B; -A¹-A²-B; or -L²-A¹-L¹-A²-L³-B.

The present disclosure provides a compound of formula:

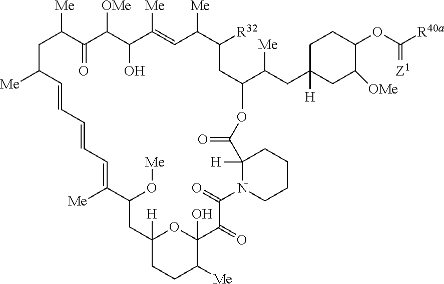

or a pharmaceutically acceptable salt or tautomer thereof, having one, two, three, or four of the following features:
a) Z¹ is O;
b) A¹ is absent;
c) L¹ is

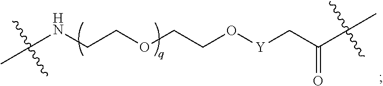

d) B is

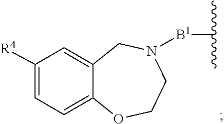

e) B¹ is

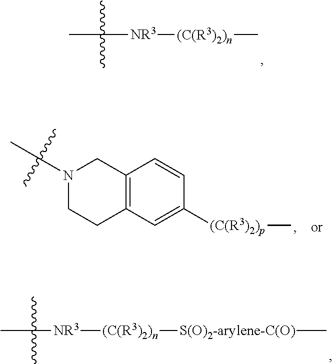

wherein the arylene is optionally substituted with alkyl, hydroxyalkyl, haloalkyl, alkoxy, halogen, or hydroxyl;
f) R⁴ is 5-12 membered heteroaryl, optionally substituted with —N(R³)₂ or —OR³; and
g) R³² is —OH.
   In the above, R^{40a} can be -A¹-L¹-A²-B; -A¹-A²-B; or -L²-A¹-L¹-A²-L³-B.

The present disclosure provides a compound of formula:

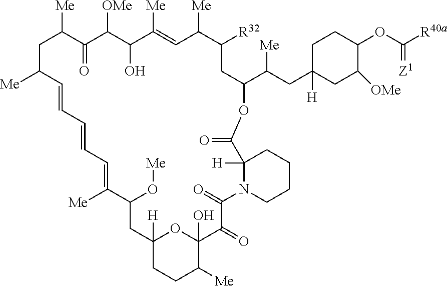

or a pharmaceutically acceptable salt or tautomer thereof, having one, two, three, or four of the following features:
a) Z¹ is O;
b) A¹ is

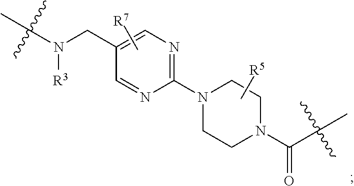

c) A² is

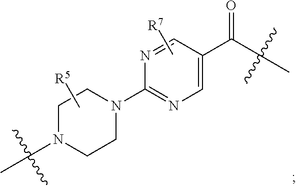

d) L¹ is

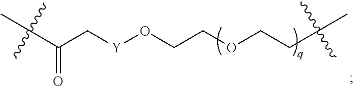

e) B is

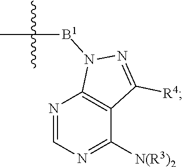

f) B¹ is

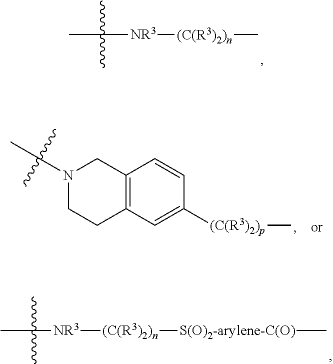

wherein the arylene is optionally substituted with alkyl, hydroxyalkyl, haloalkyl, alkoxy, halogen, or hydroxyl;
g) R⁴ is 5-12 membered heteroaryl, optionally substituted with —N(R³)₂ or —OR³; and
h) R³² is —OH.
   In the above, R^{40a} can be -A¹-L¹-A²-B; -A¹-A²-B; or -L²-A¹-L¹-A²-L³-B.

In certain embodiments, in Formula Ia or Ic, R^{40a} is any organic moiety, which may have a molecular weight (e.g. the sum of the atomic masses of the atoms of the substituent) of less than 15 g/mol, 50 g/mol, 100 g/mol, 150 g/mol, 200 g/mol, 250 g/mol, 300 g/mol, 350 g/mol, 400 g/mol, 450 g/mol, or 500 g/mol.

In certain embodiments, the present disclosure provides for a compound selected from below or a pharmaceutically acceptable salt or tautomer thereof,

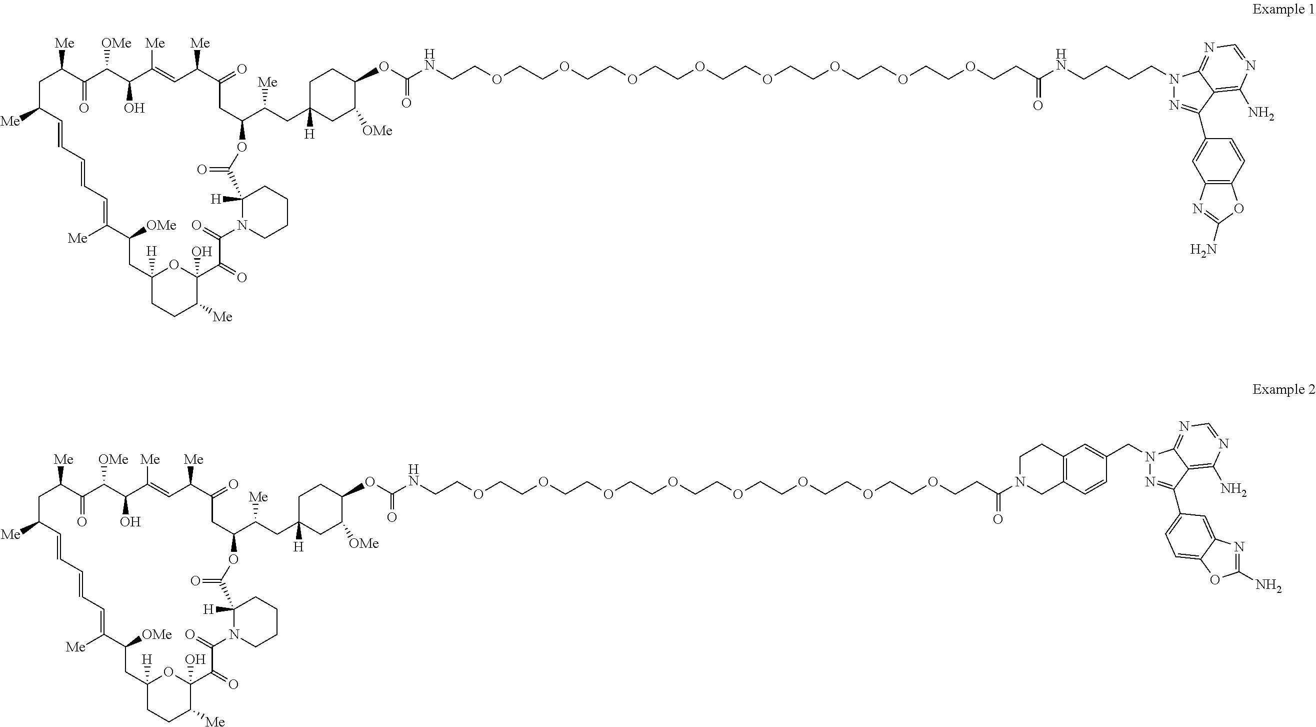

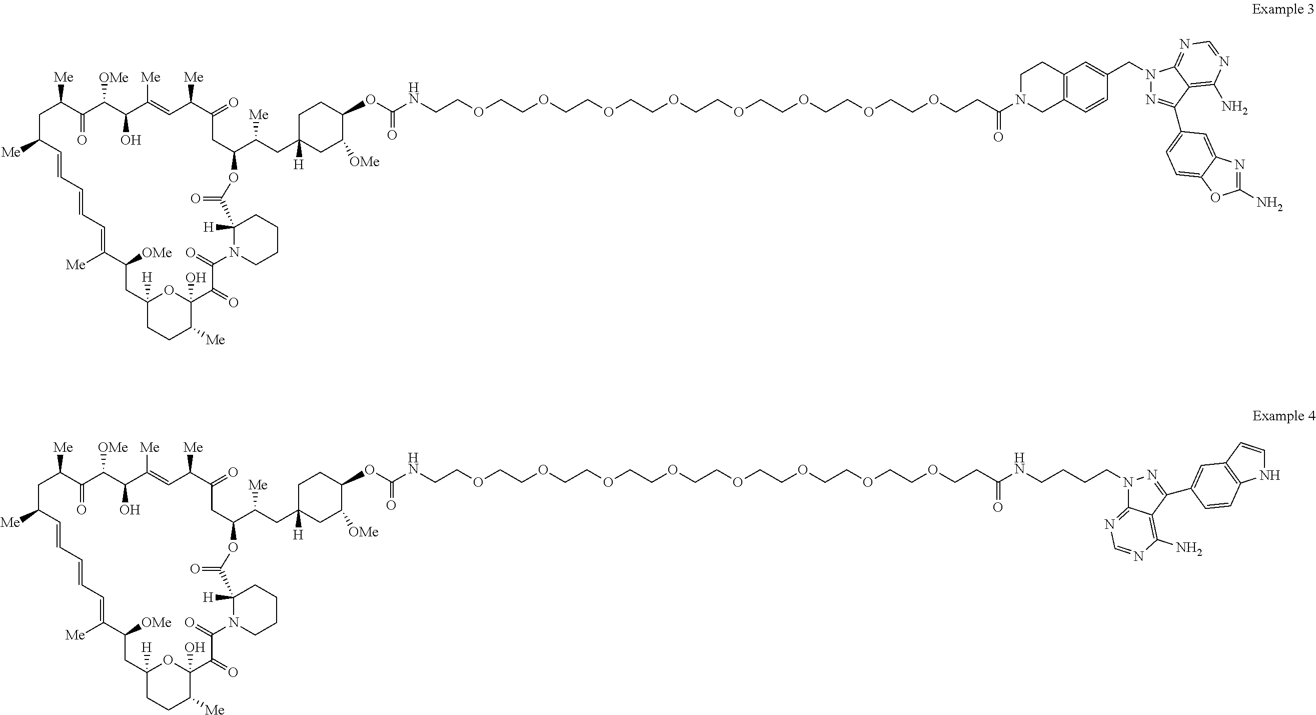

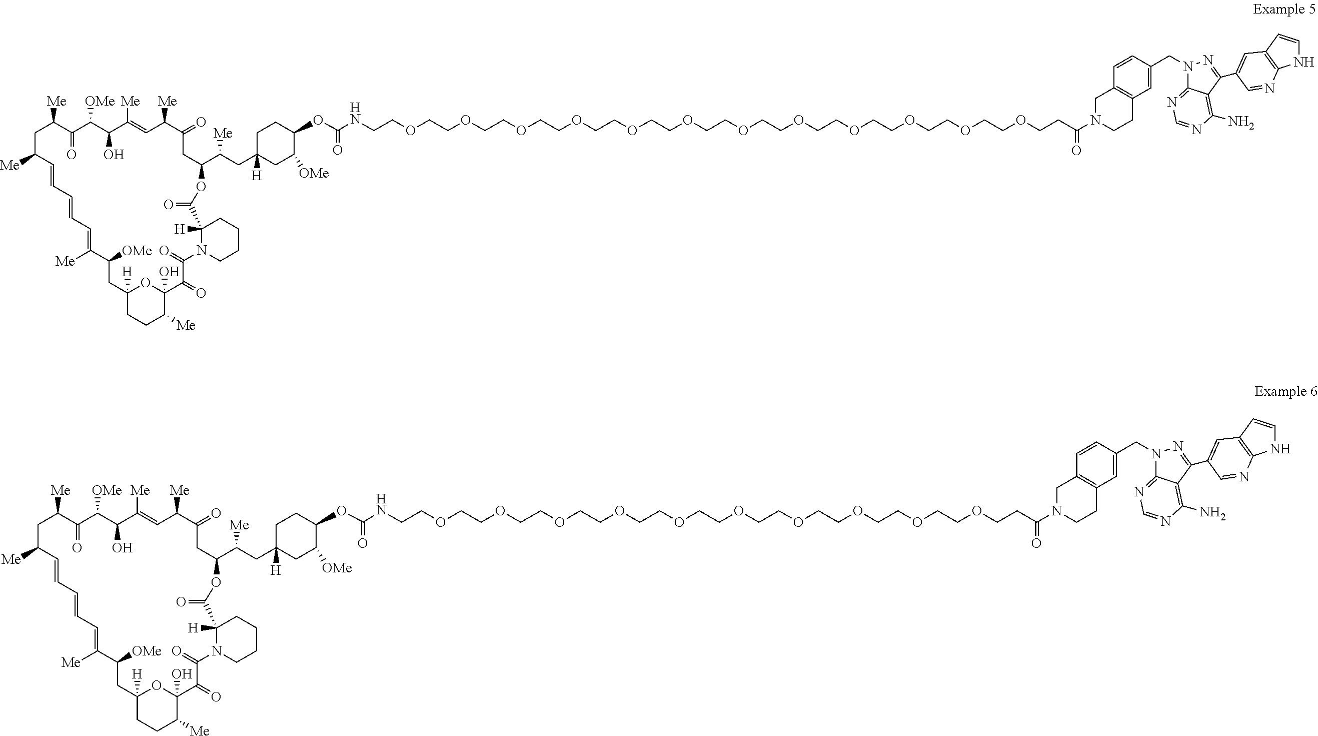

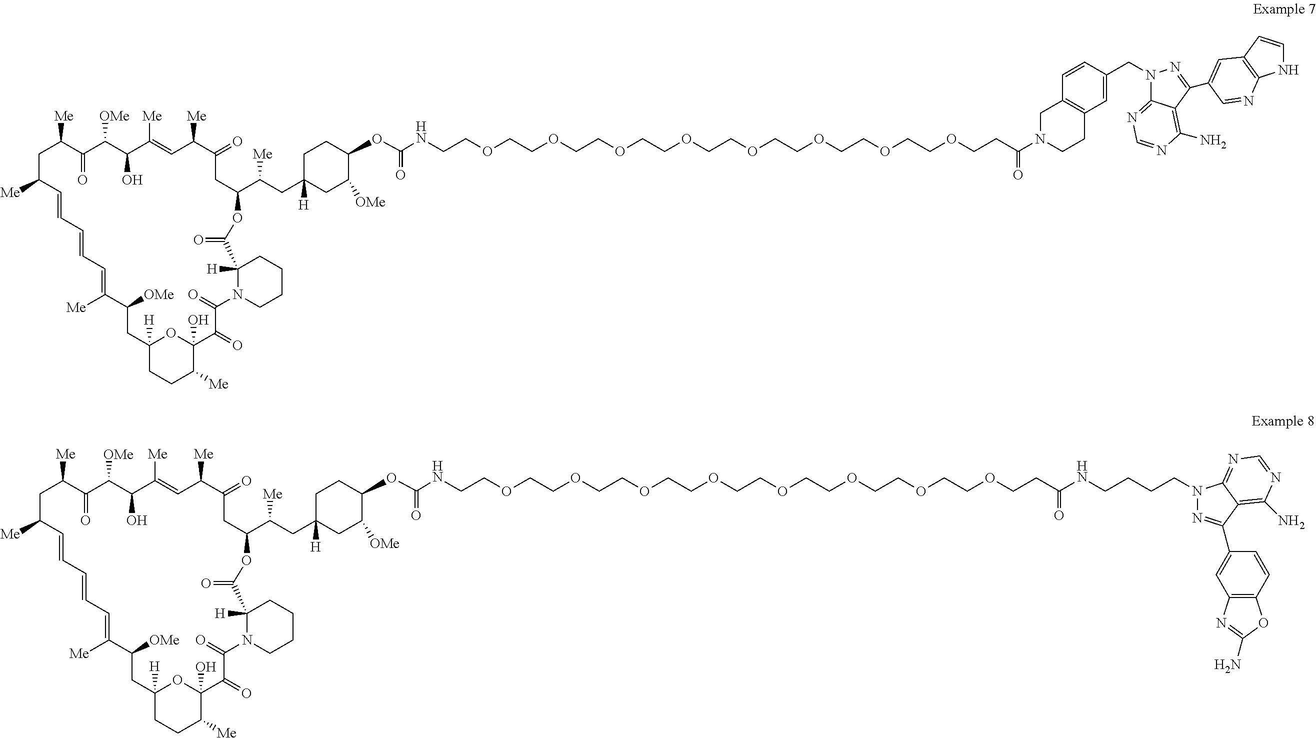

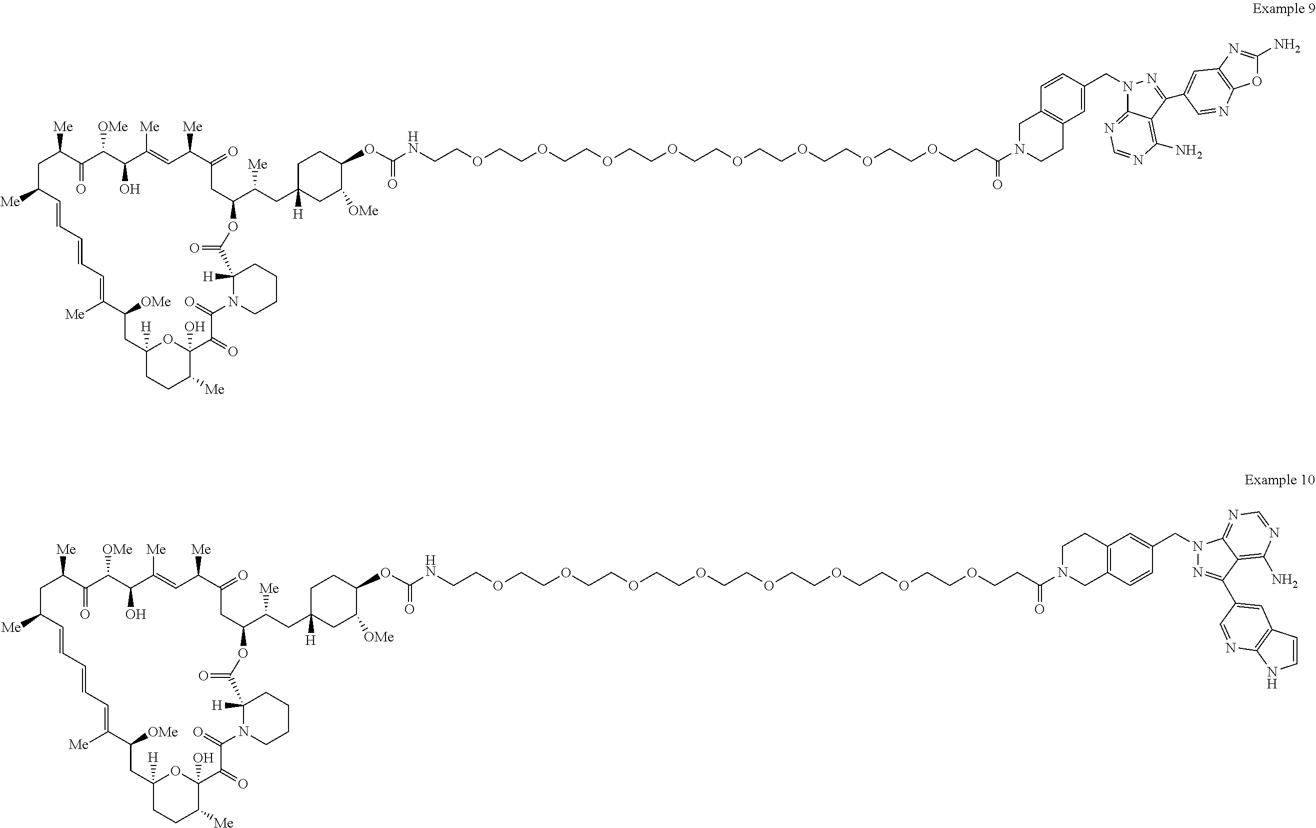

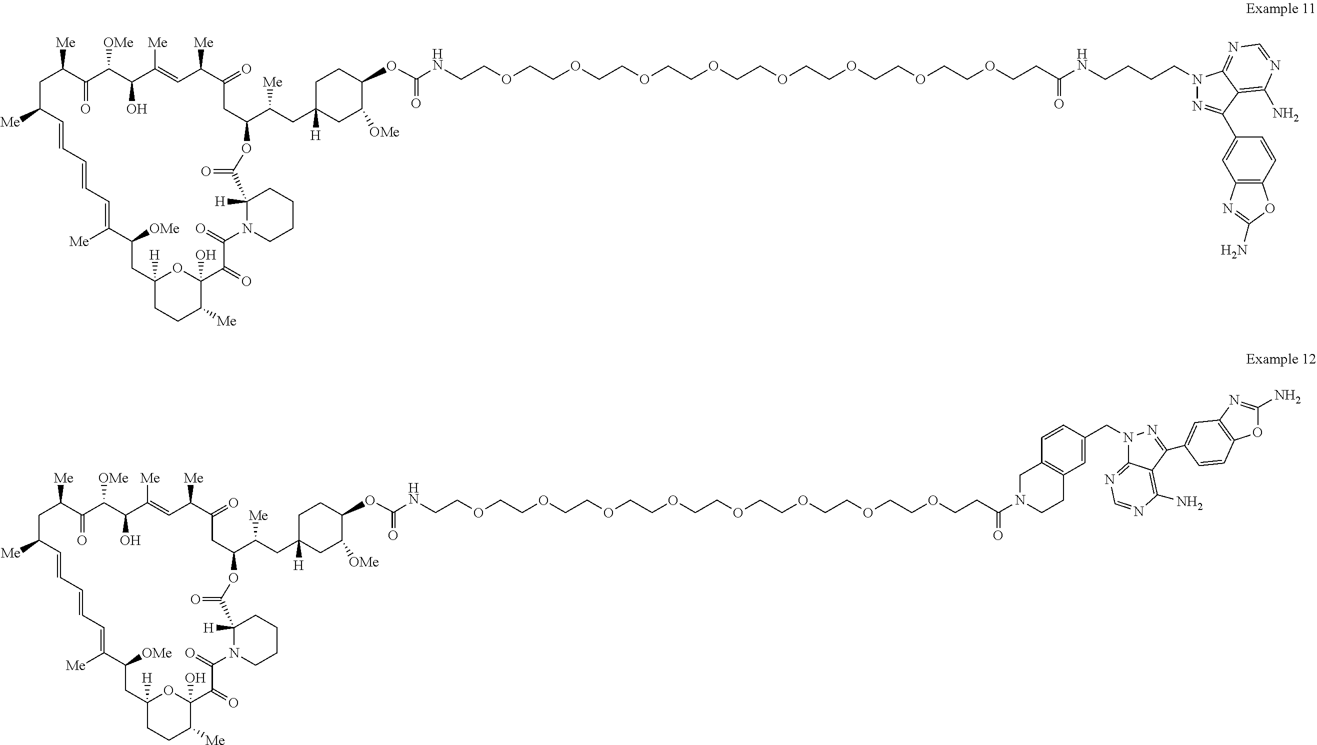

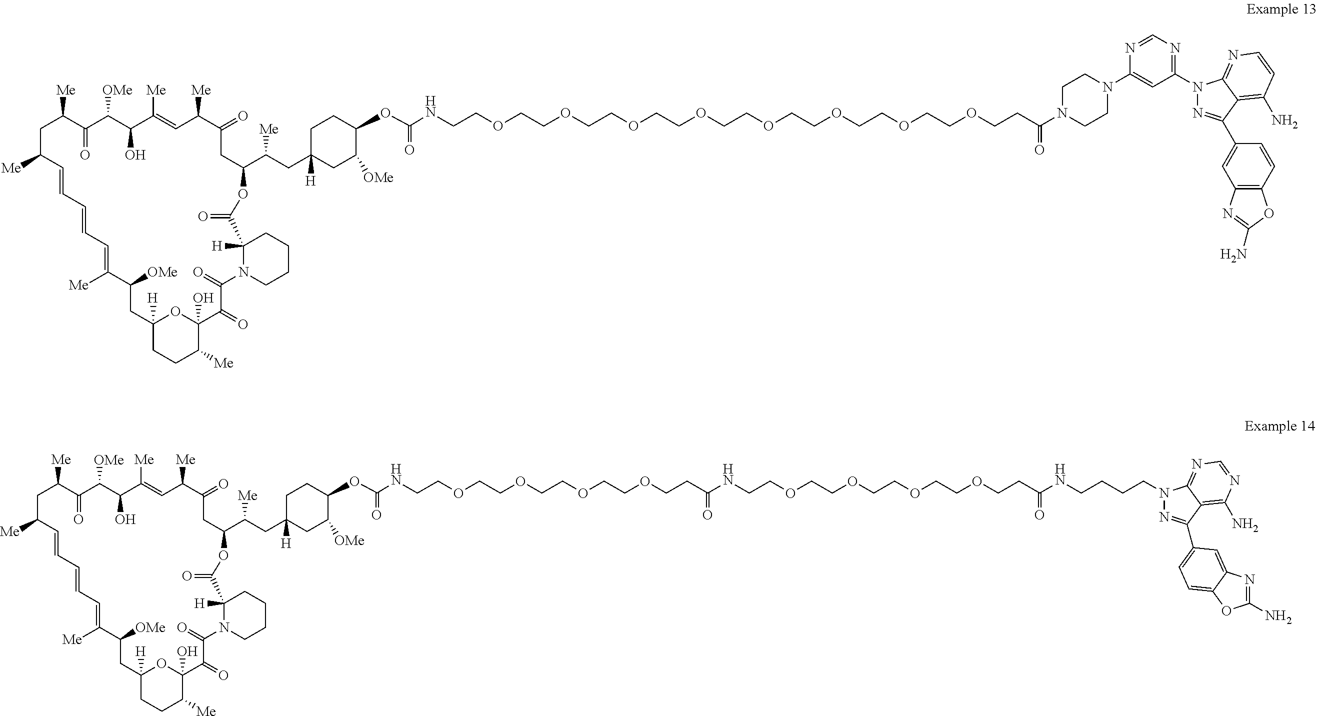

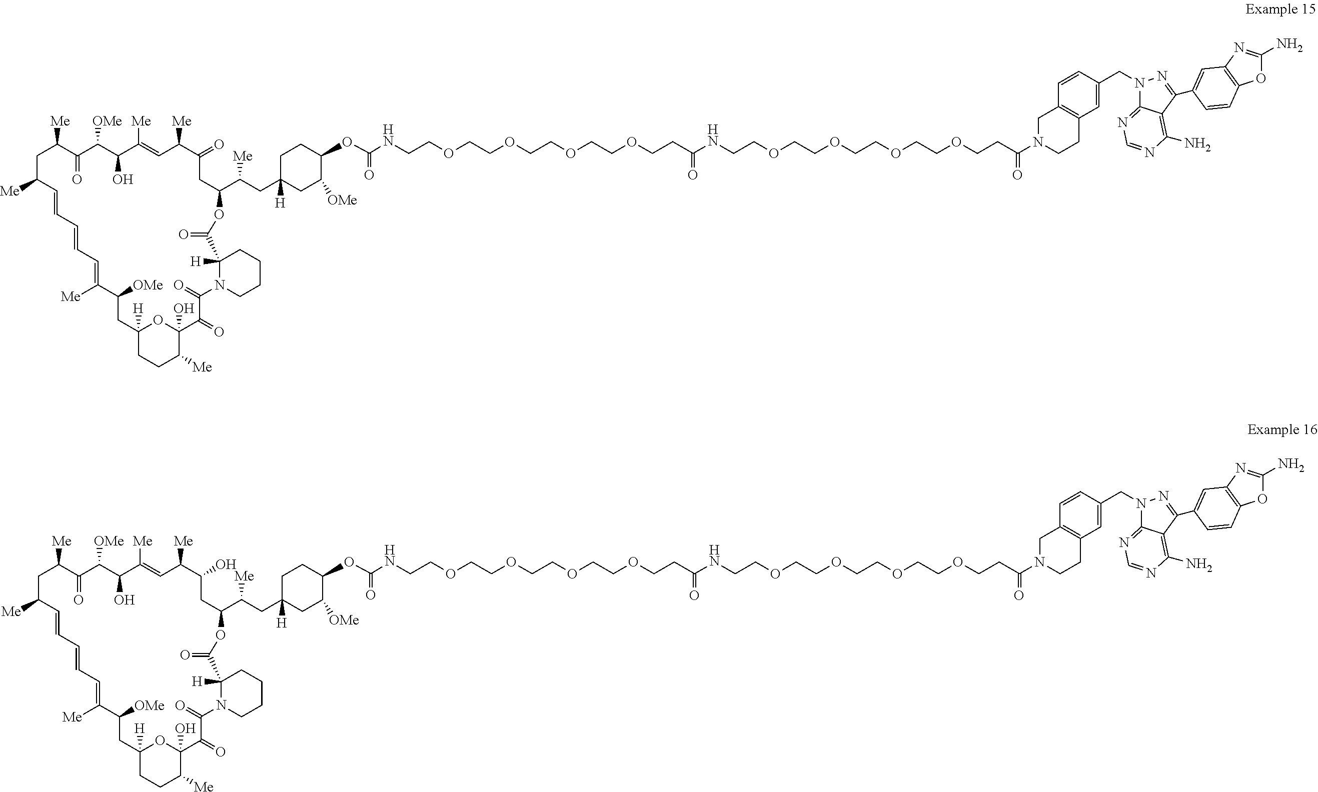

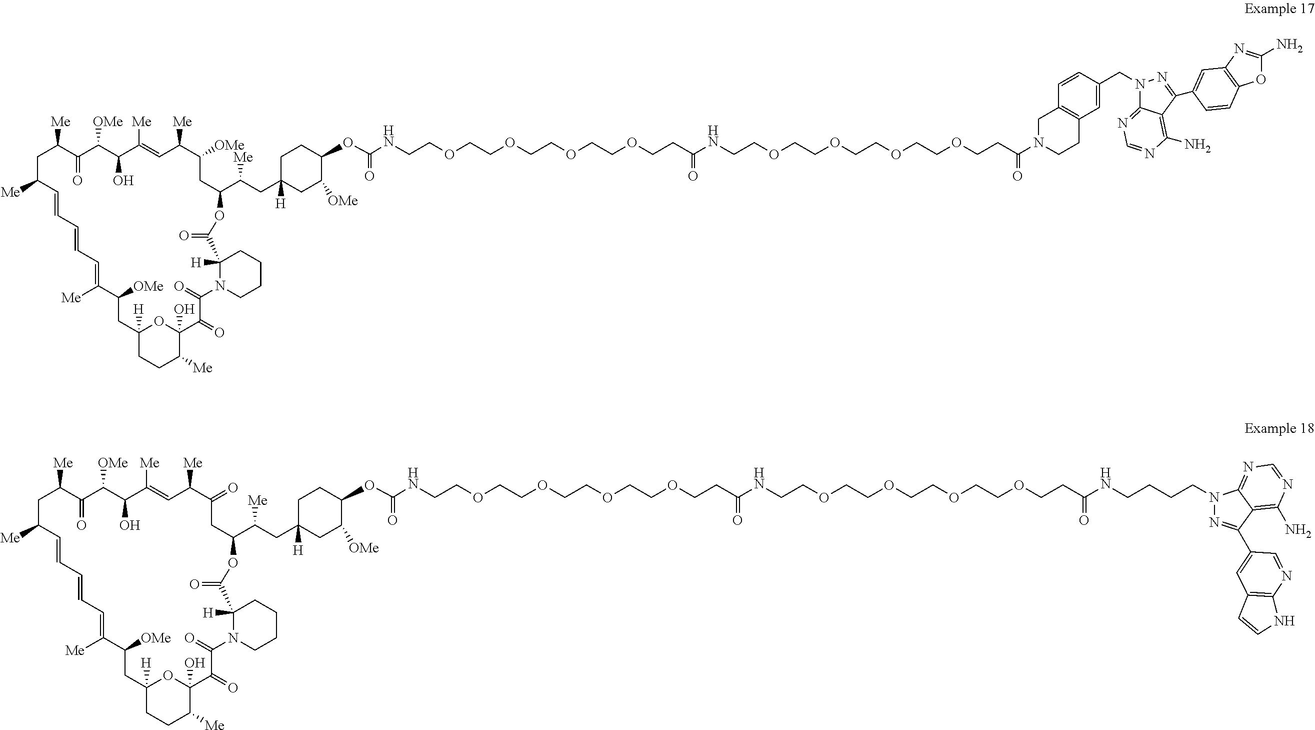

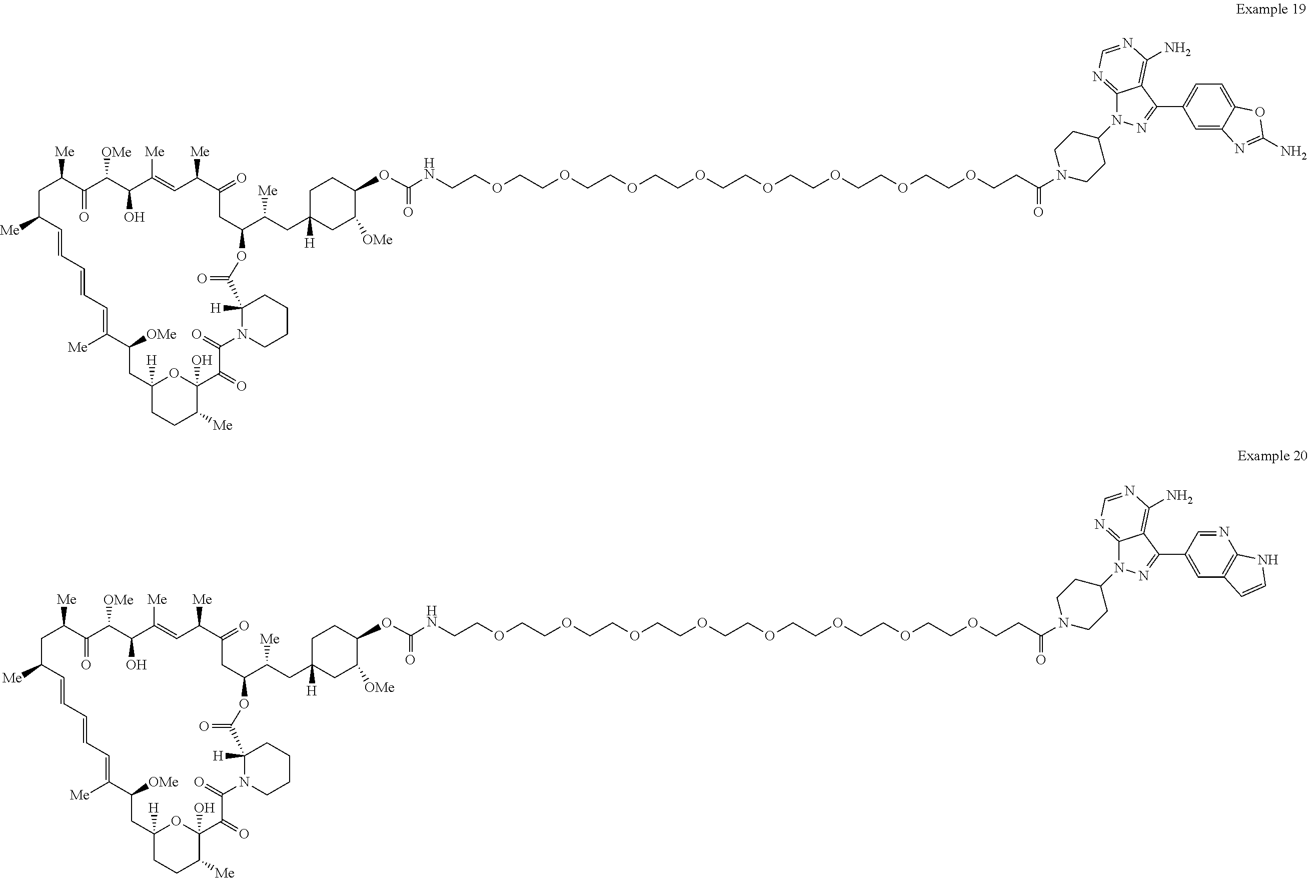

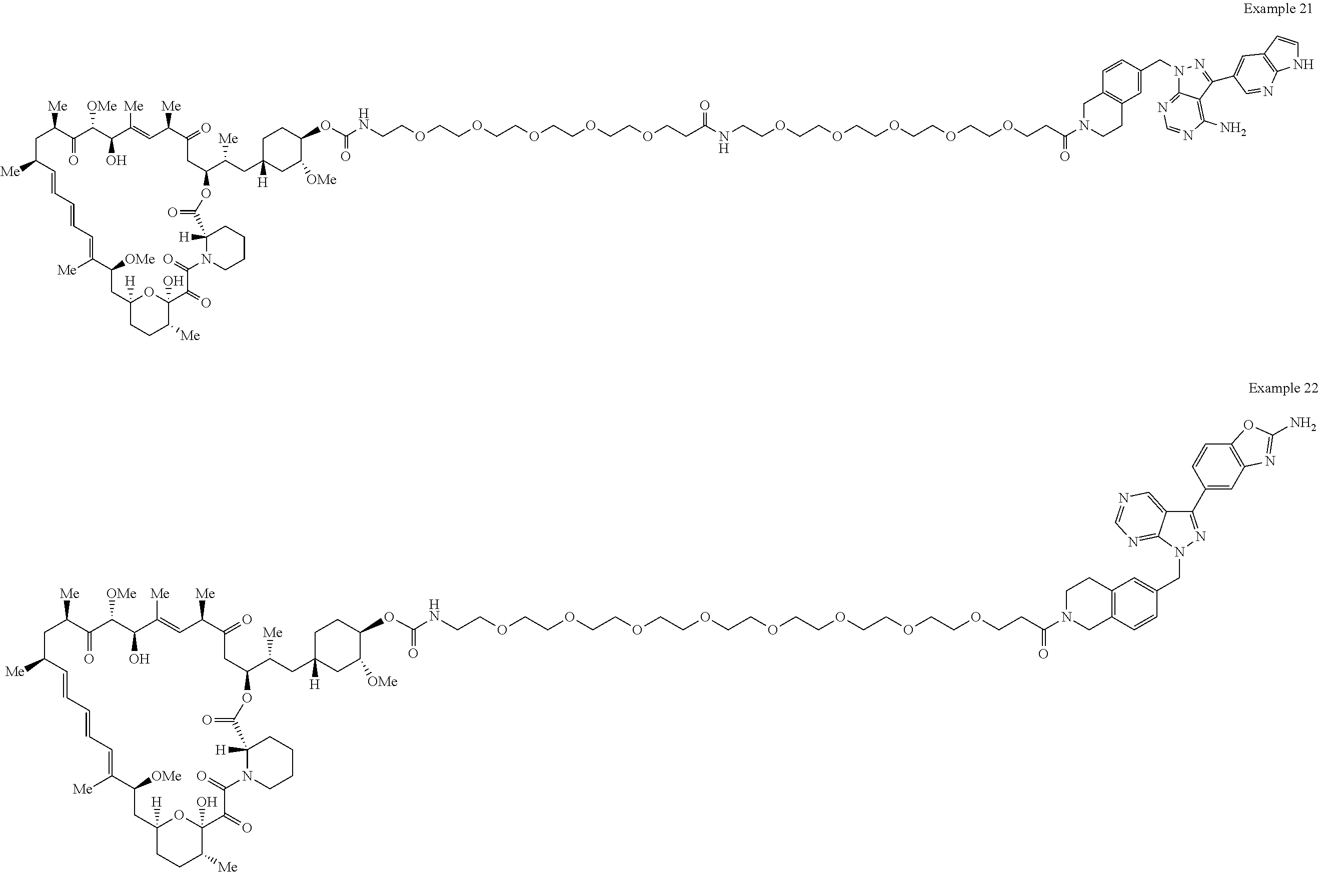

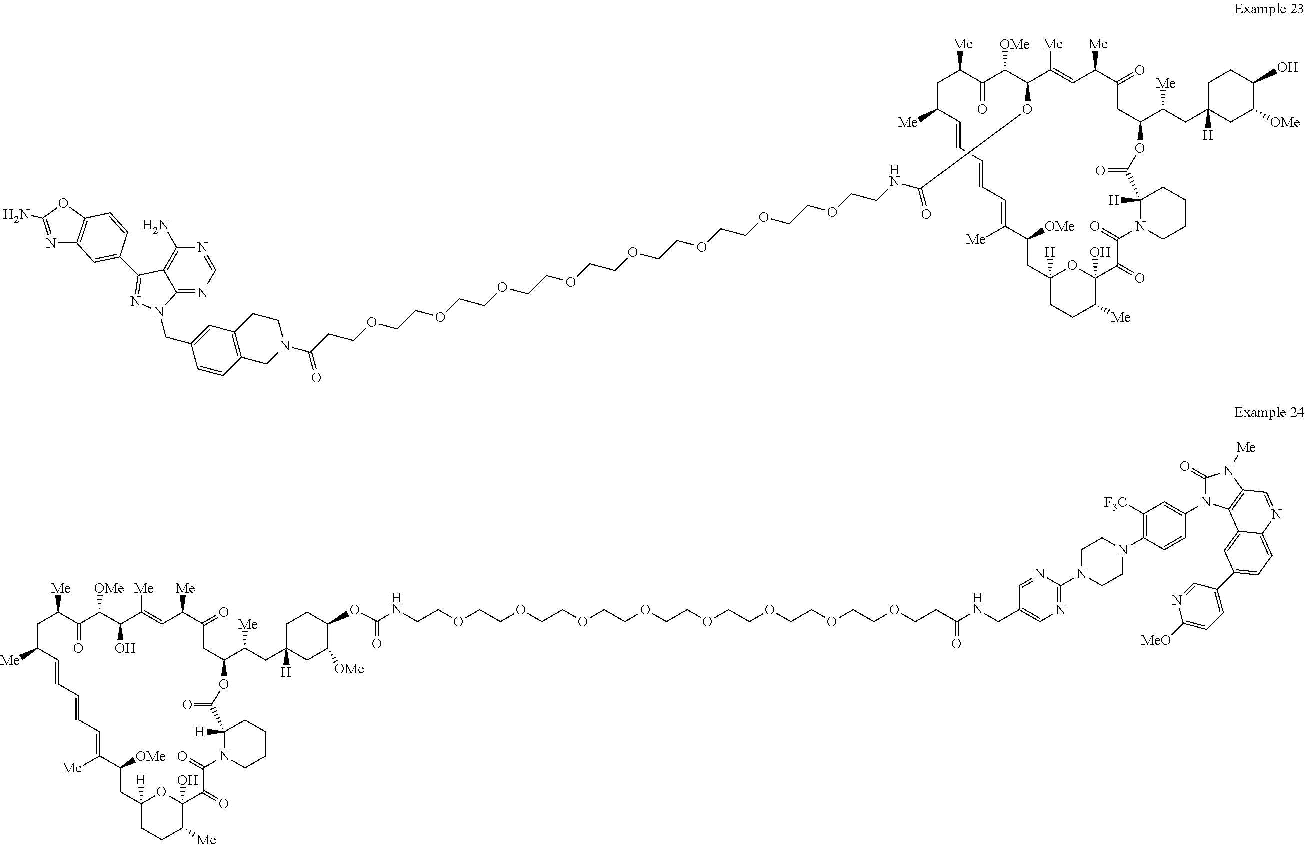

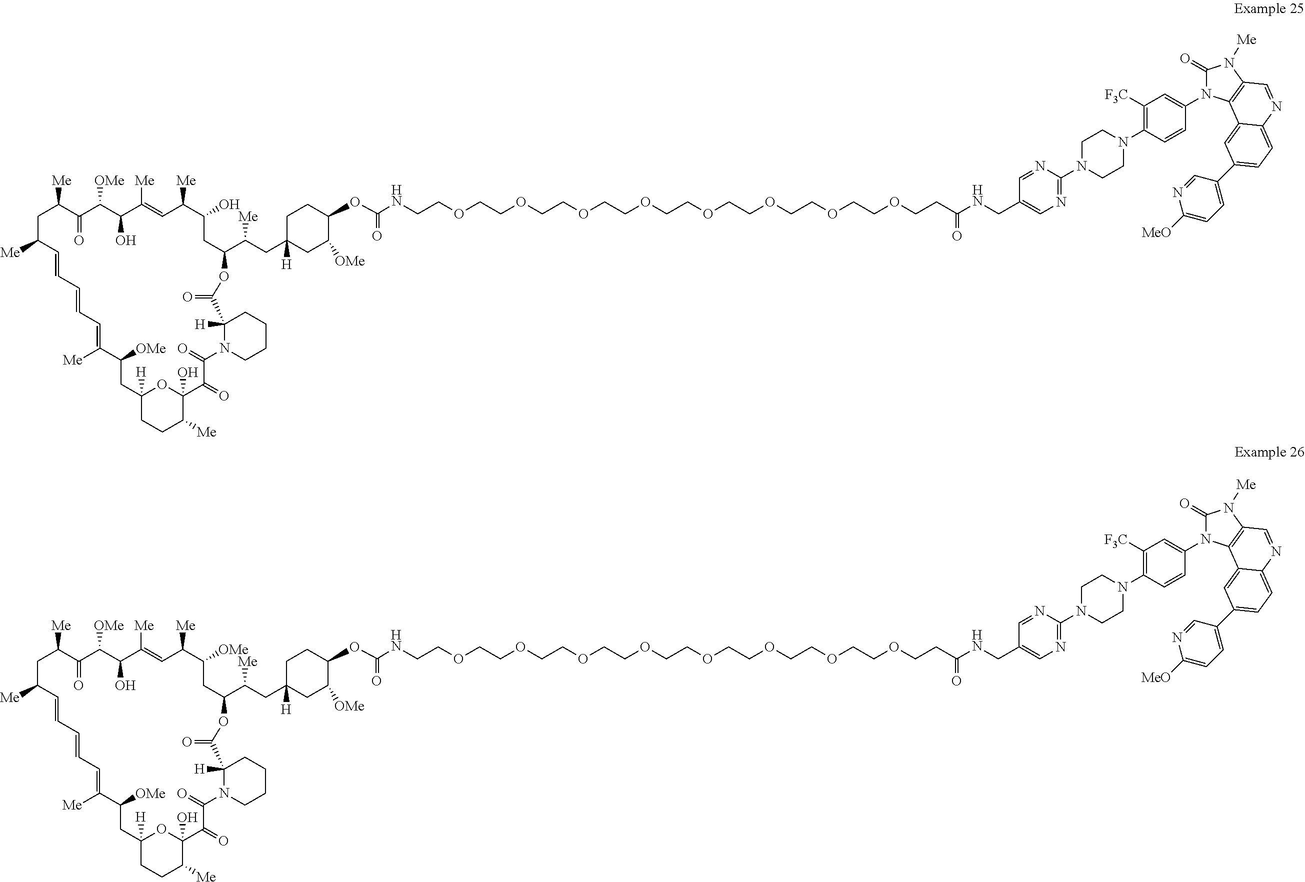

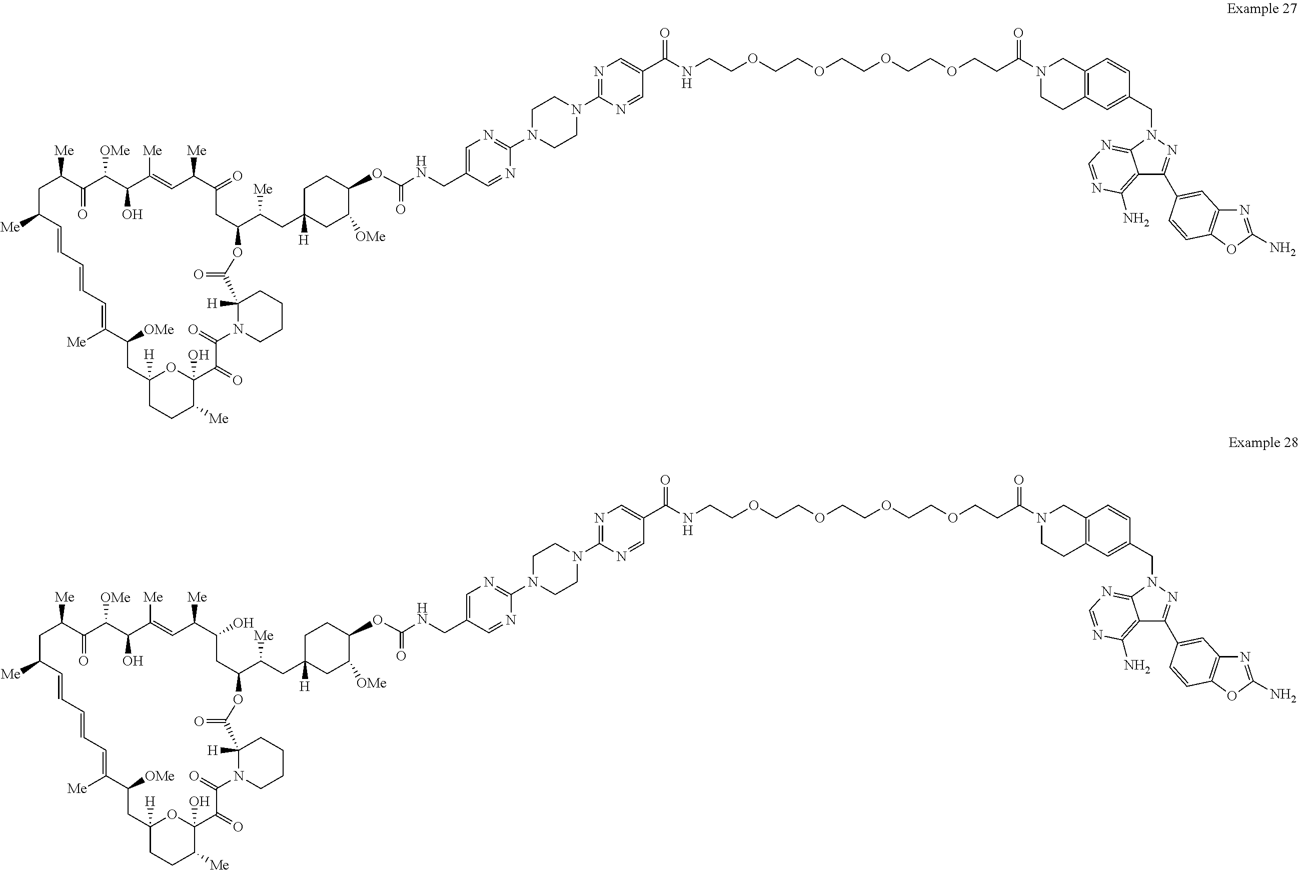

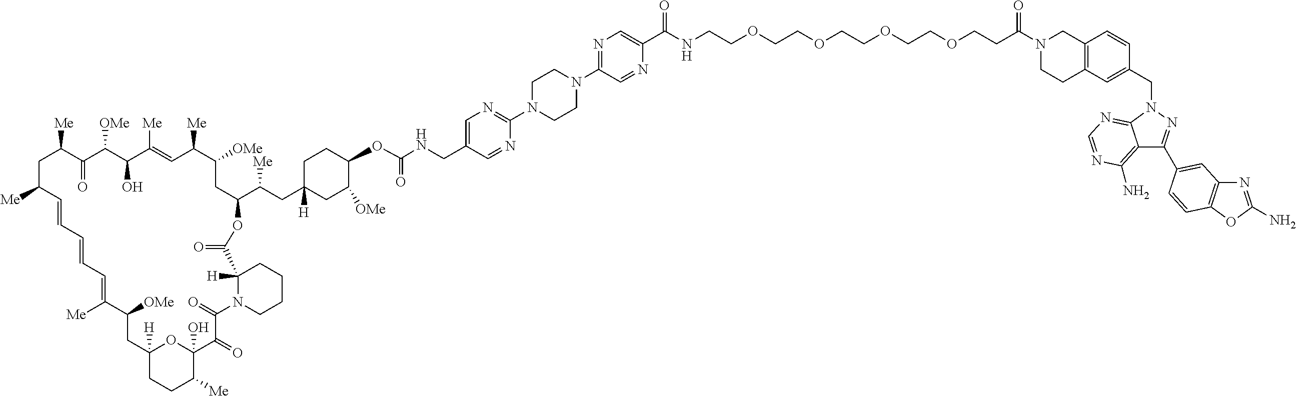 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5cnc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

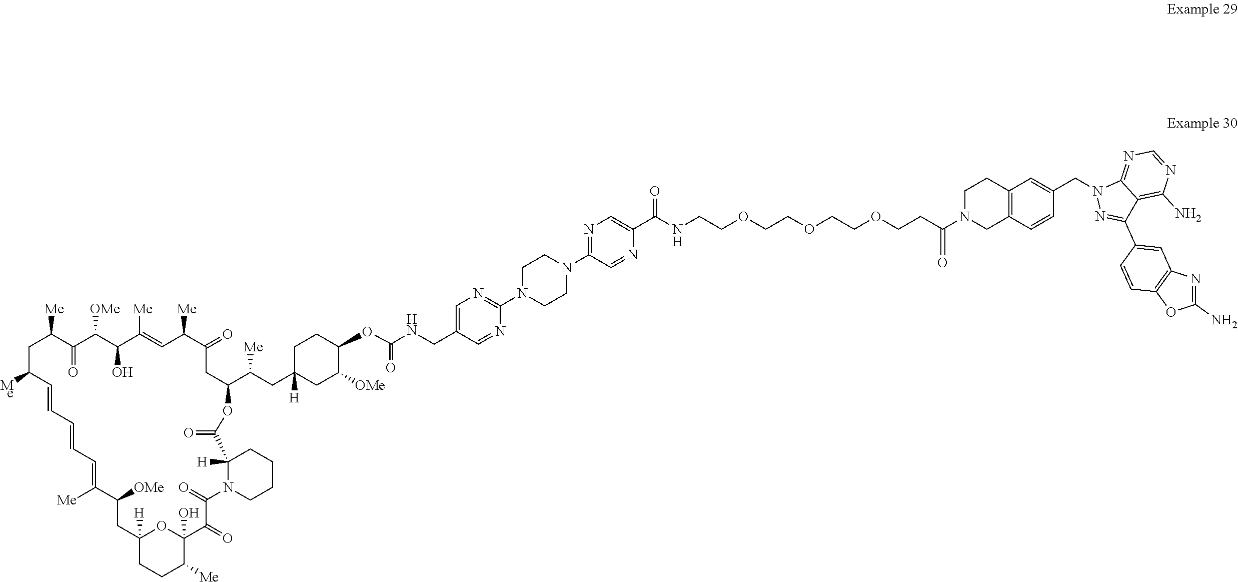 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5cnc(C(=O)NCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

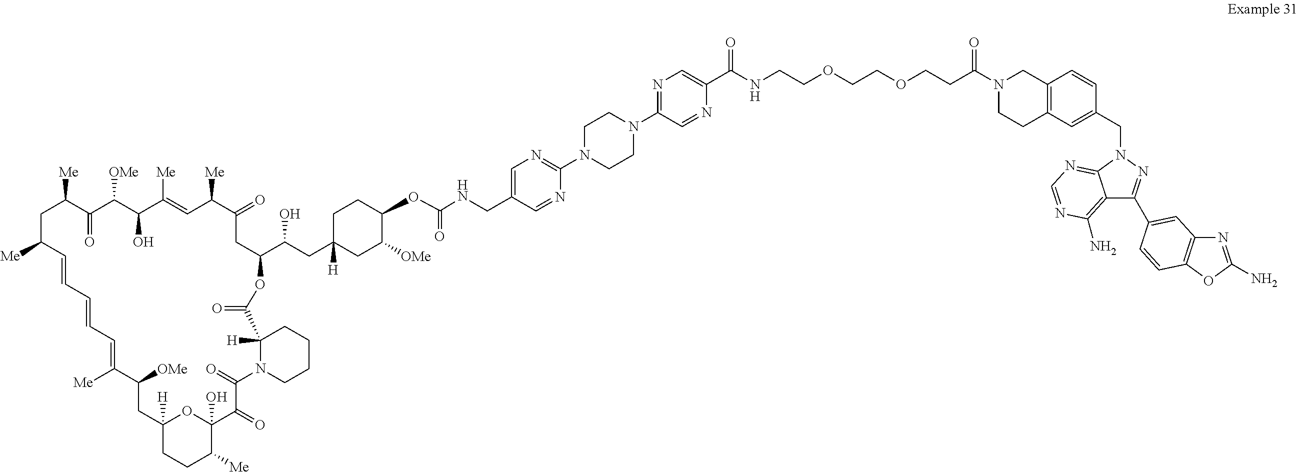 [SMILES: CO[C@H]1C[C@@H]2CC[C@@H](C)[C@@](O)(O2)C(=O)C(=O)N2CCCC[C@H]2C(=O)O[C@H]([C@H](O)C[C@@H]2CC[C@@H](OC(=O)NCc3cnc(N4CCN(c5cnc(C(=O)NCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

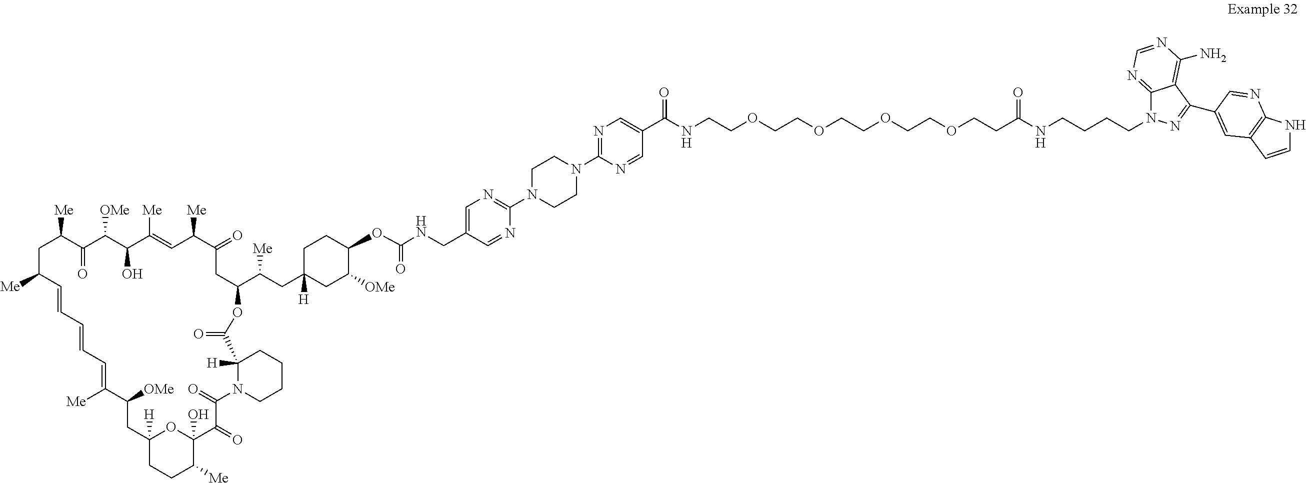 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

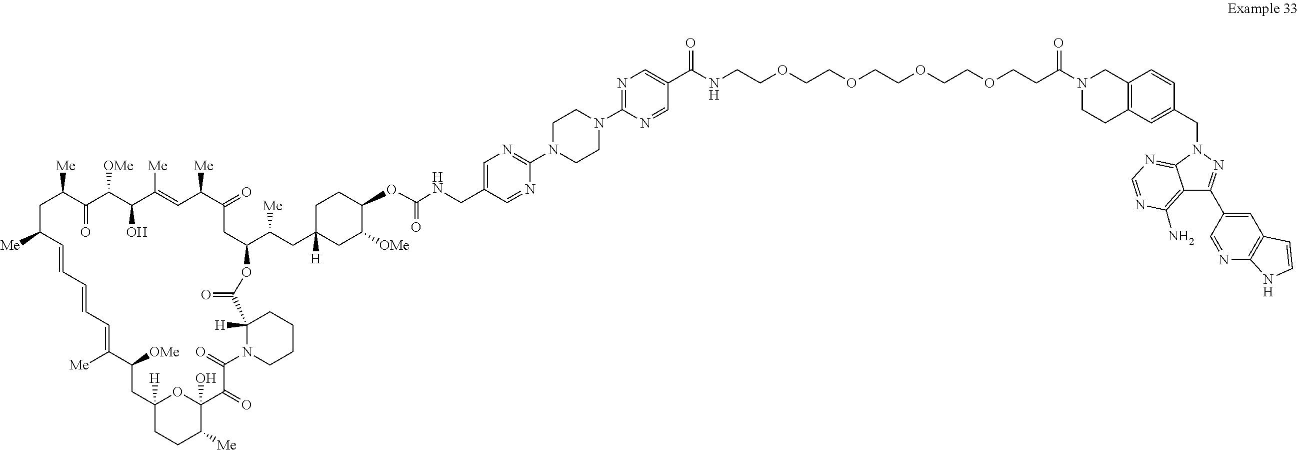 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

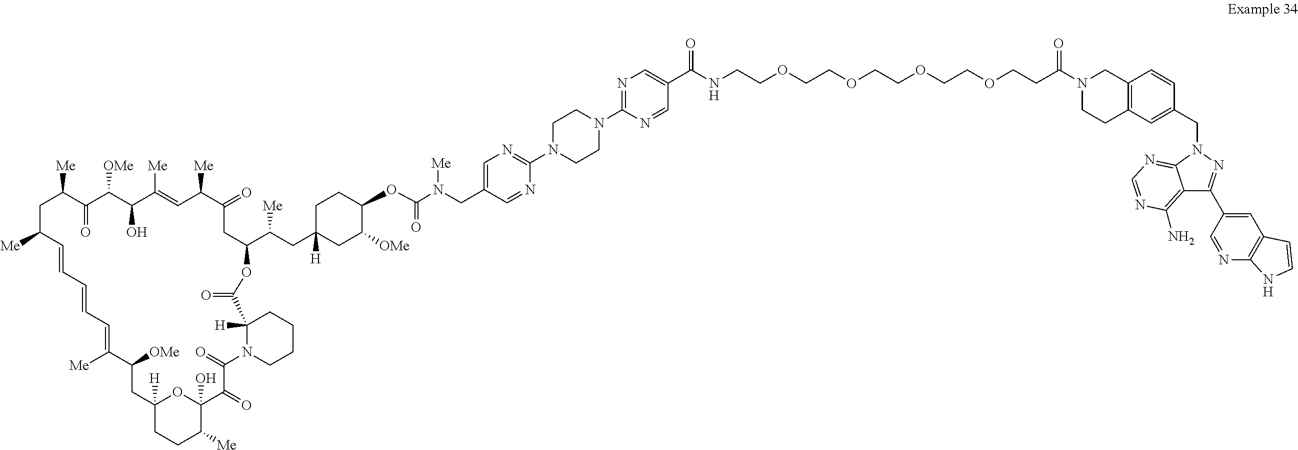 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)Cc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

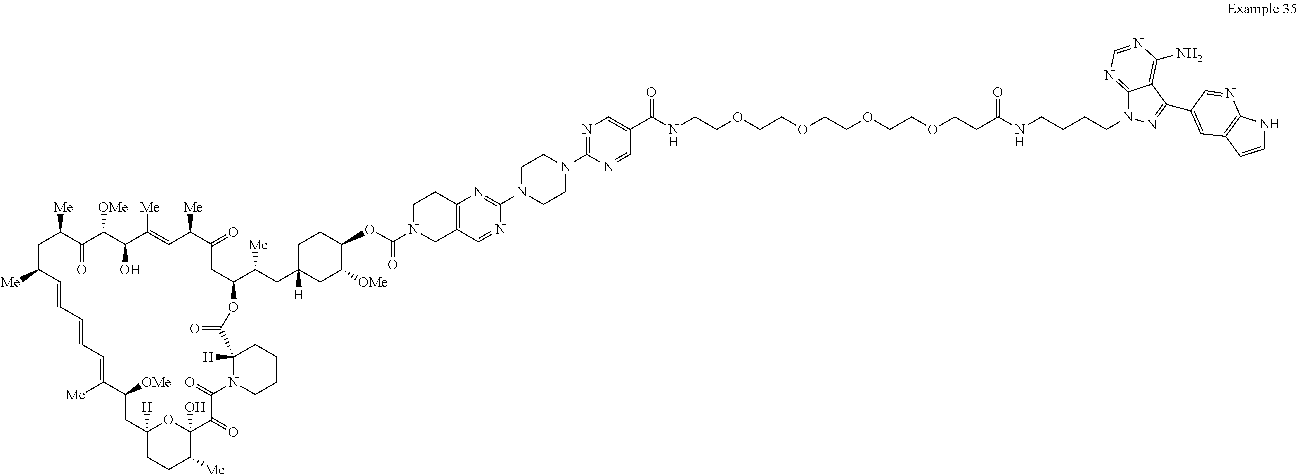 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)NCCCCn7nc(-c8cnc9[nH]ccc9c8)c8c(N)ncnc87)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

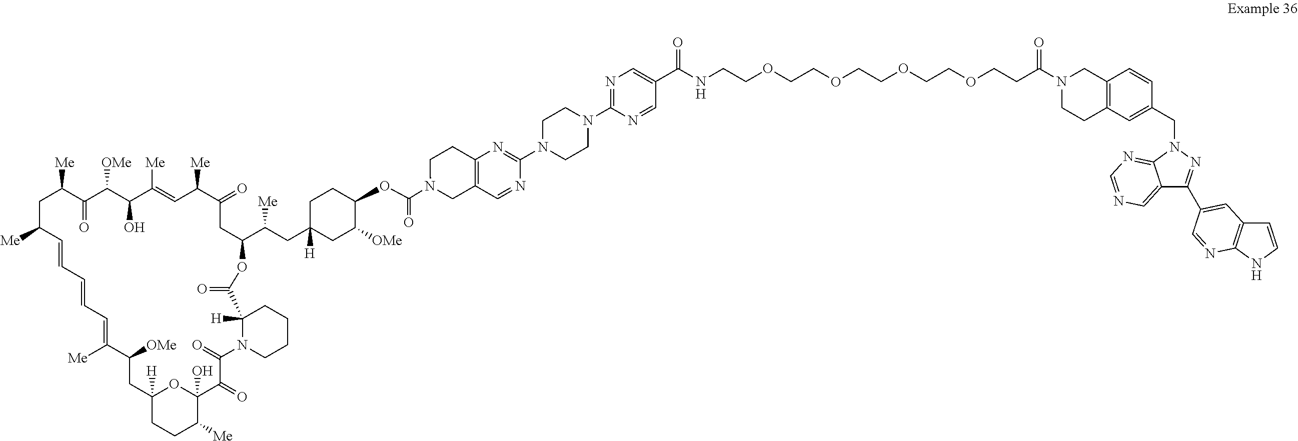 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10cncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

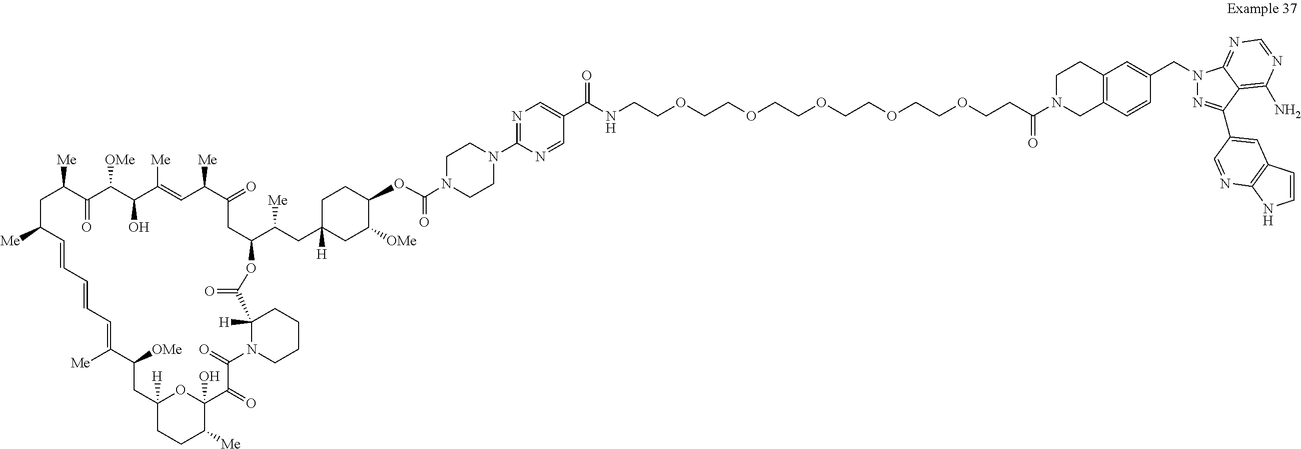 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(c4ncc(C(=O)NCCOCCOCCOCCOCCOCCC(=O)N5CCc6cc(Cn7nc(-c8cnc9[nH]ccc9c8)c8c(N)ncnc87)ccc6C5)cn4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C]

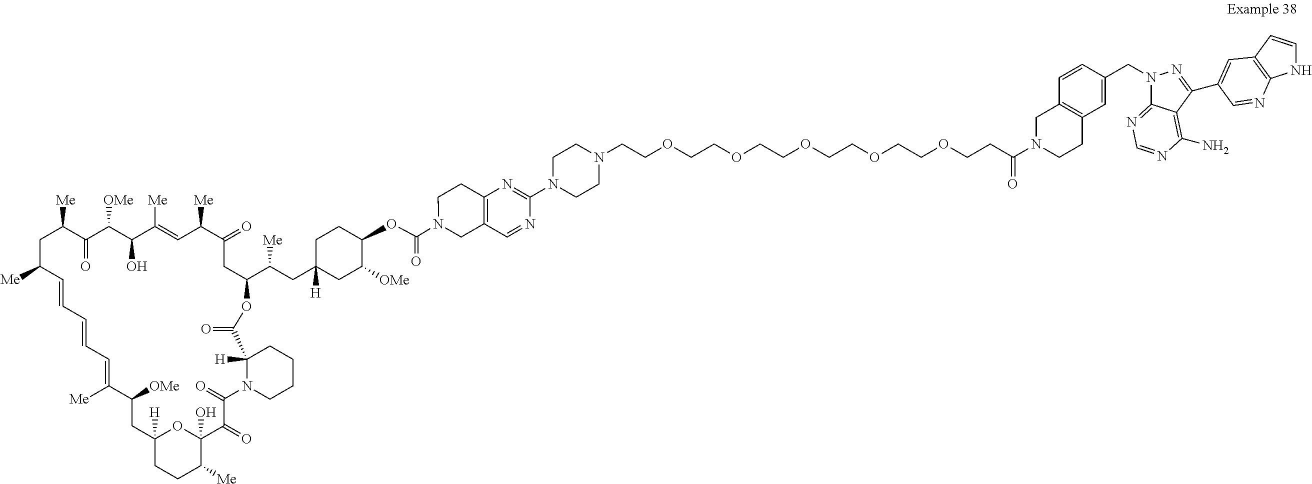 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCOCCOCCOCCOCCC(=O)N6CCc7cc(Cn8nc(-c9cnc%10[nH]ccc%10c9)c9c(N)ncnc98)ccc7C6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

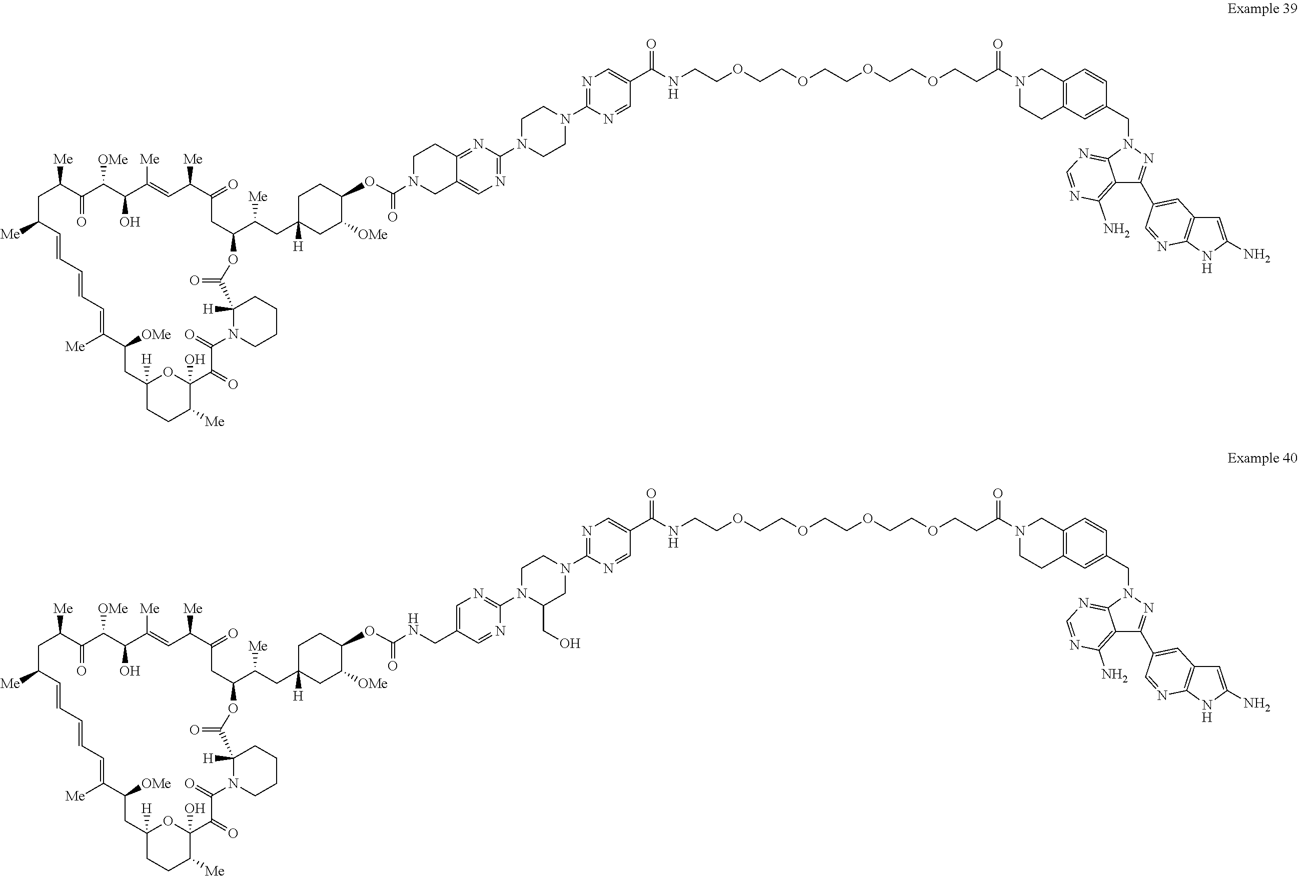

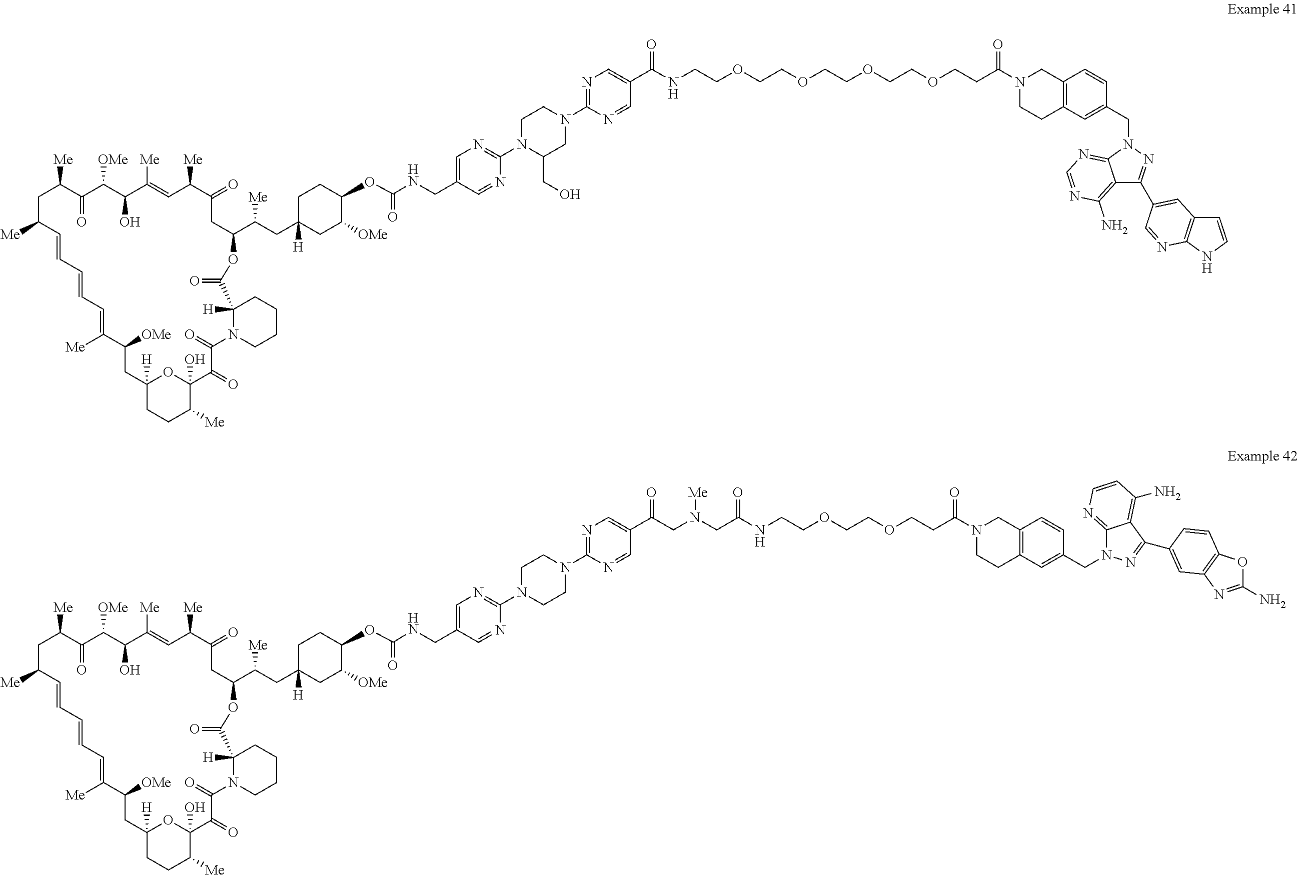

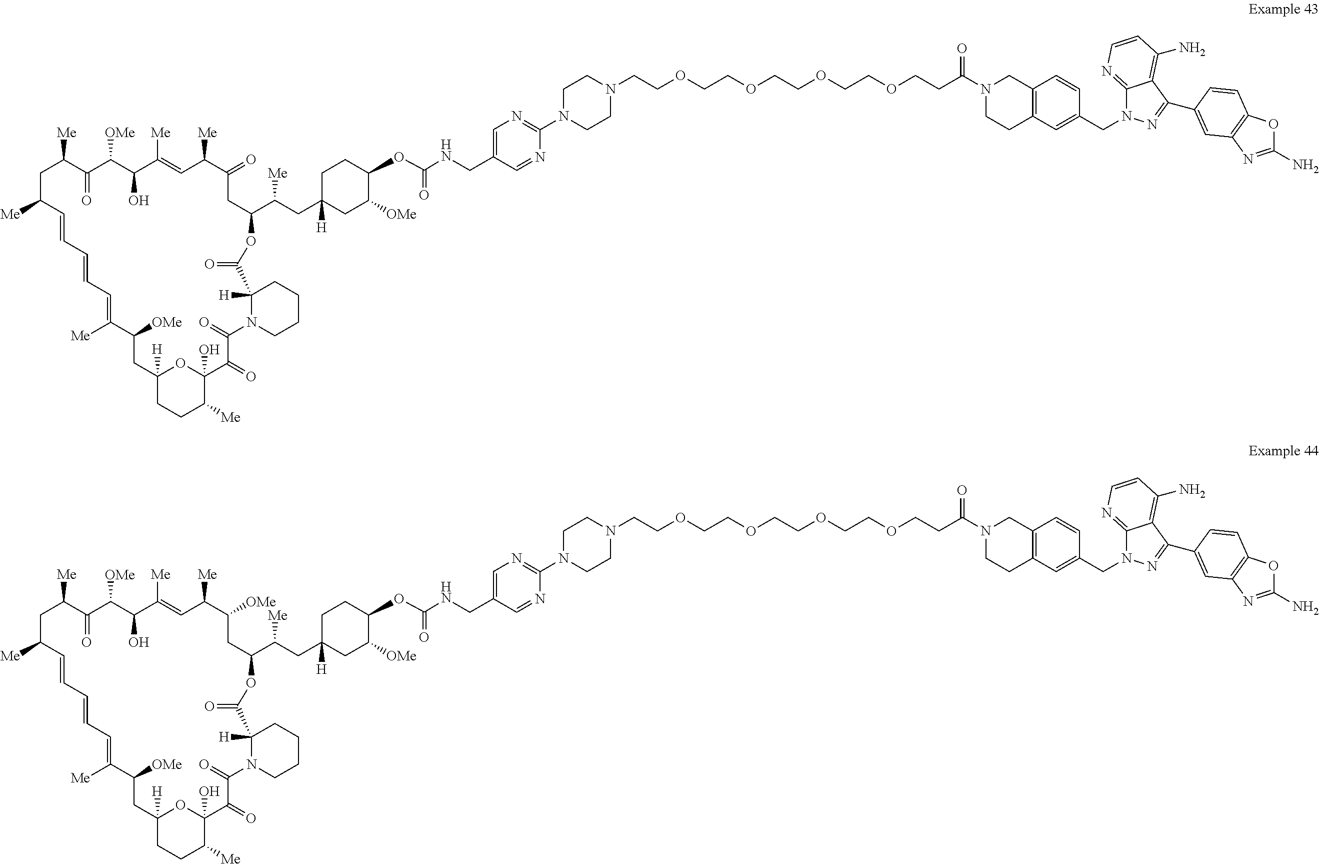

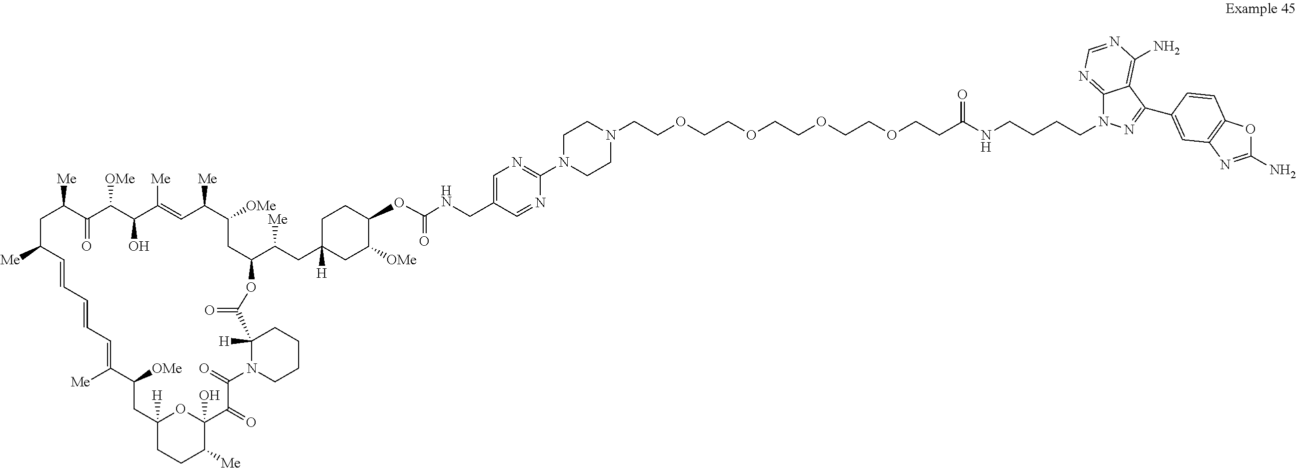 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)NCCCCn5nc(-c6ccc7oc(N)nc7c6)c6c(N)ncnc65)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

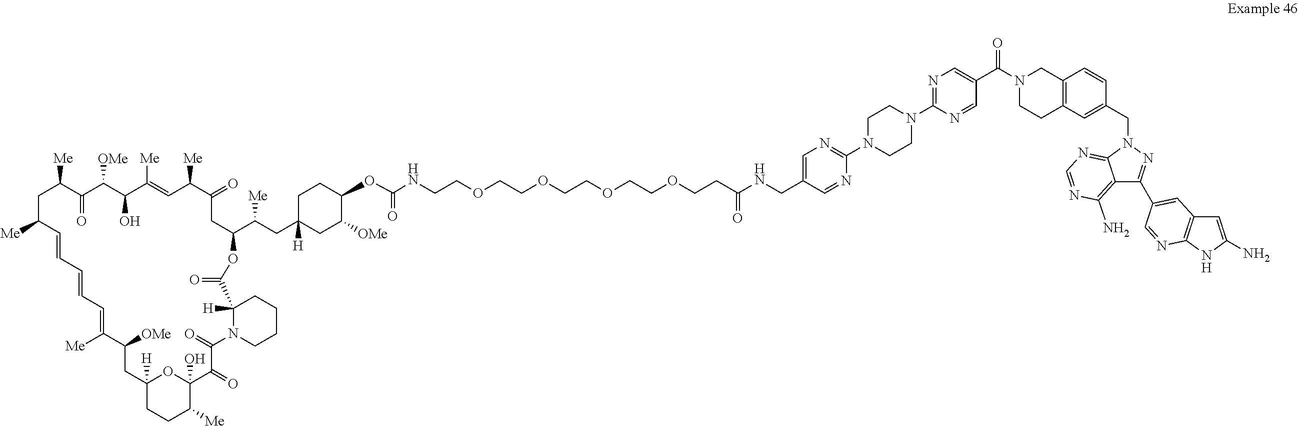 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9cnc%10[nH]c(N)cc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

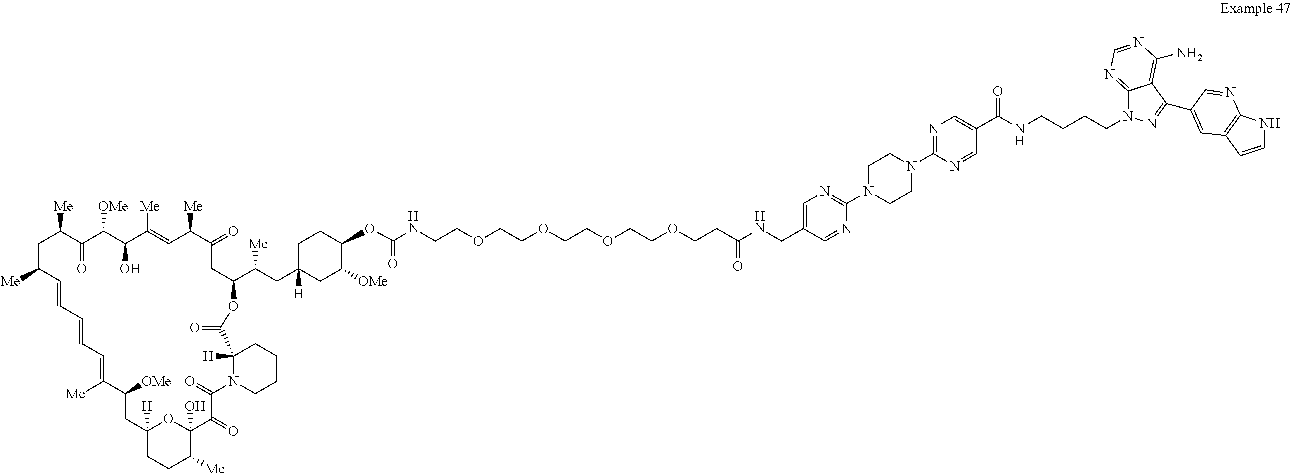 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

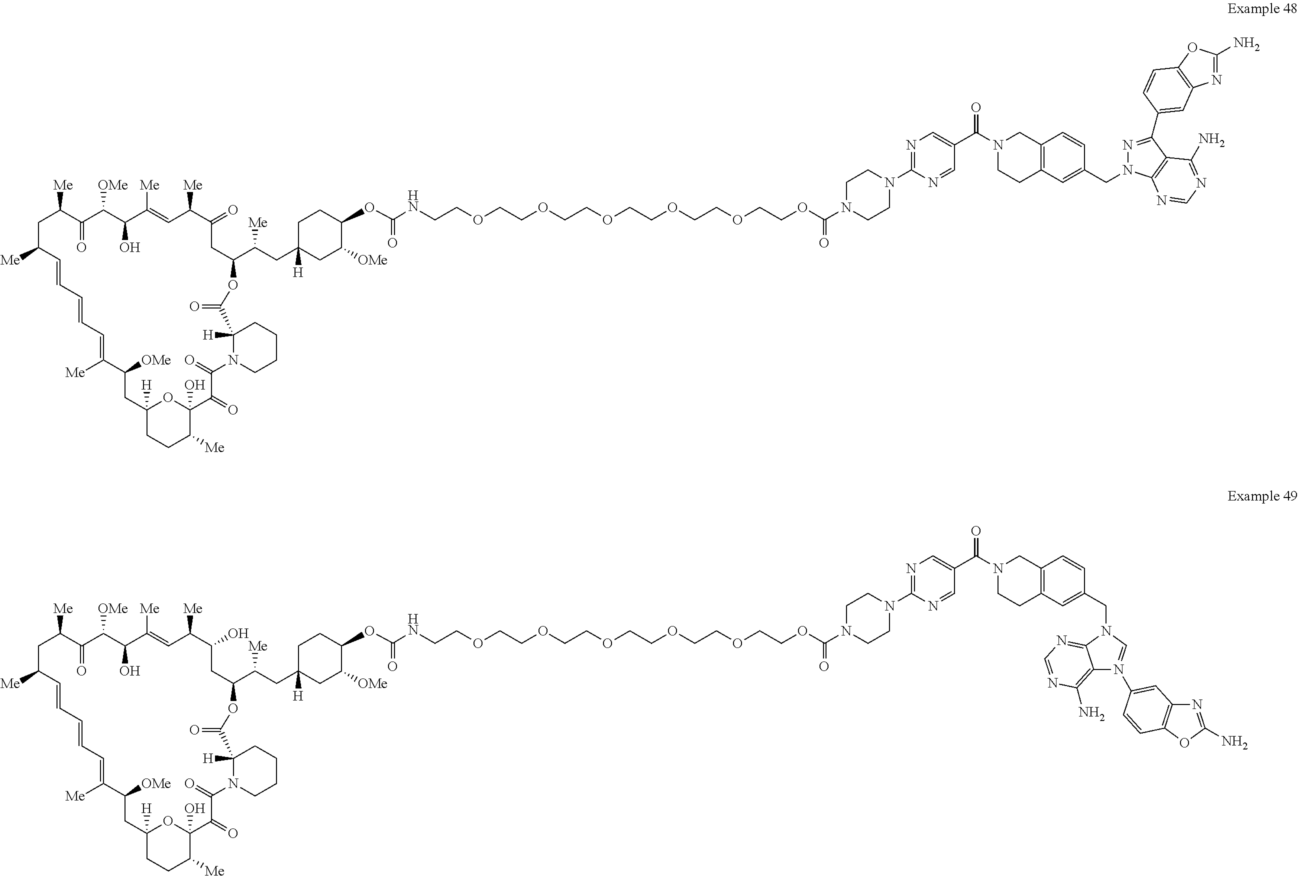

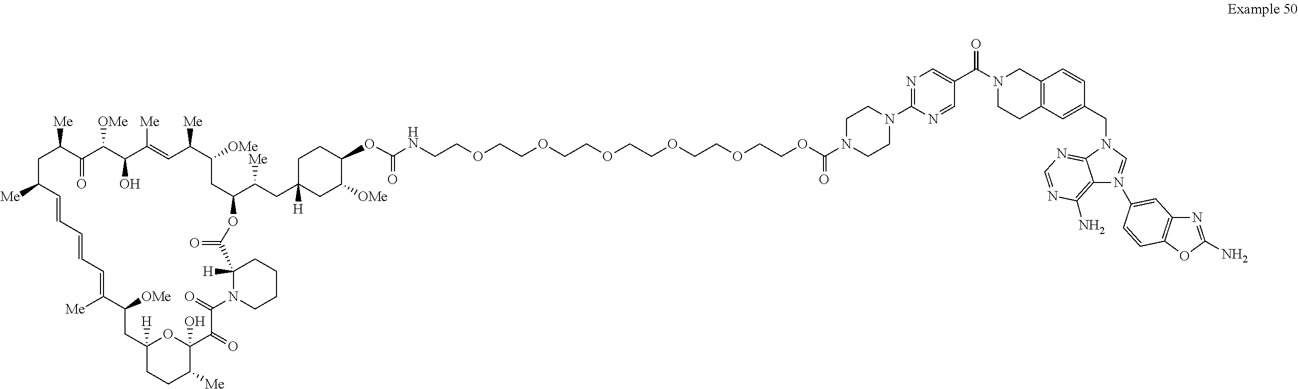

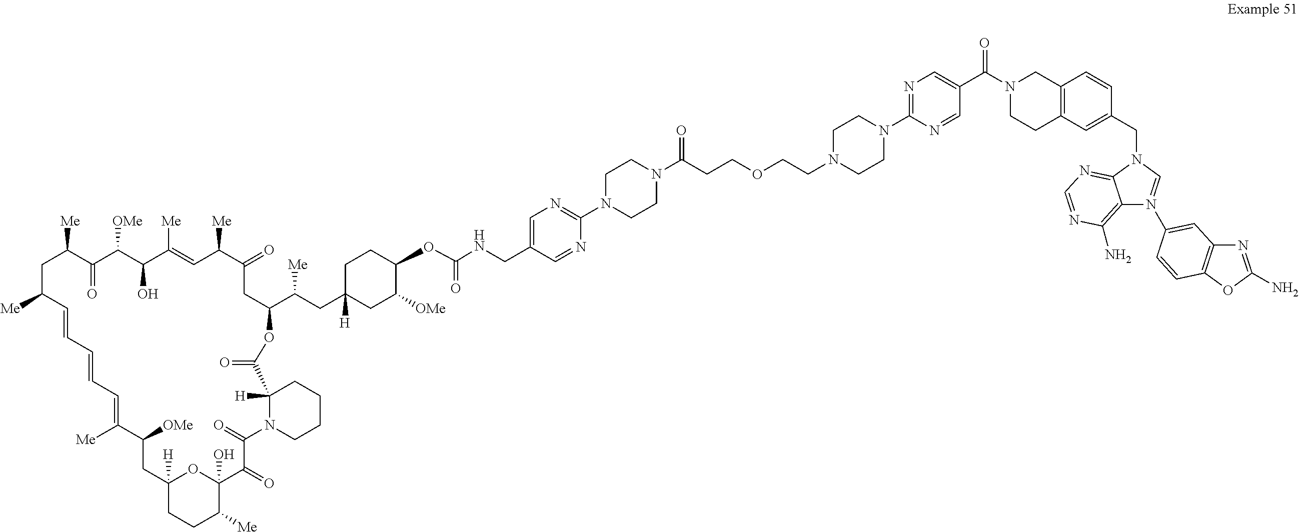

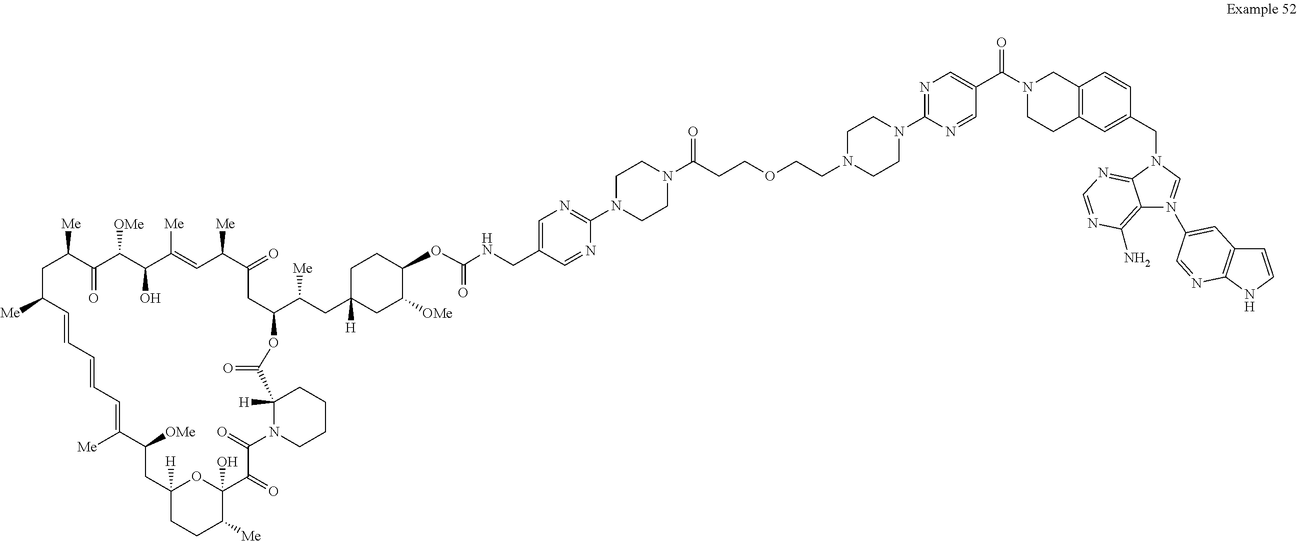

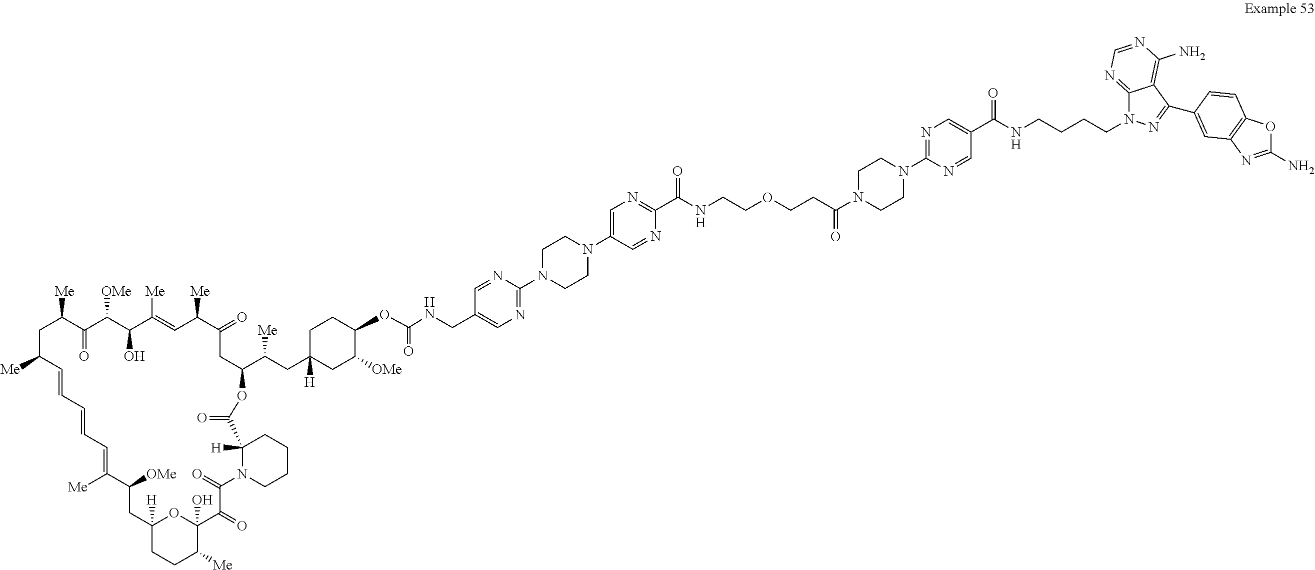 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5cnc(C(=O)NCCOCCC(=O)N6CCN(c7ncc(C(=O)NCCCCn8nc(-c9ccc%10oc(N)nc%10c9)c9c(N)ncnc98)cn7)CC6)nc5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

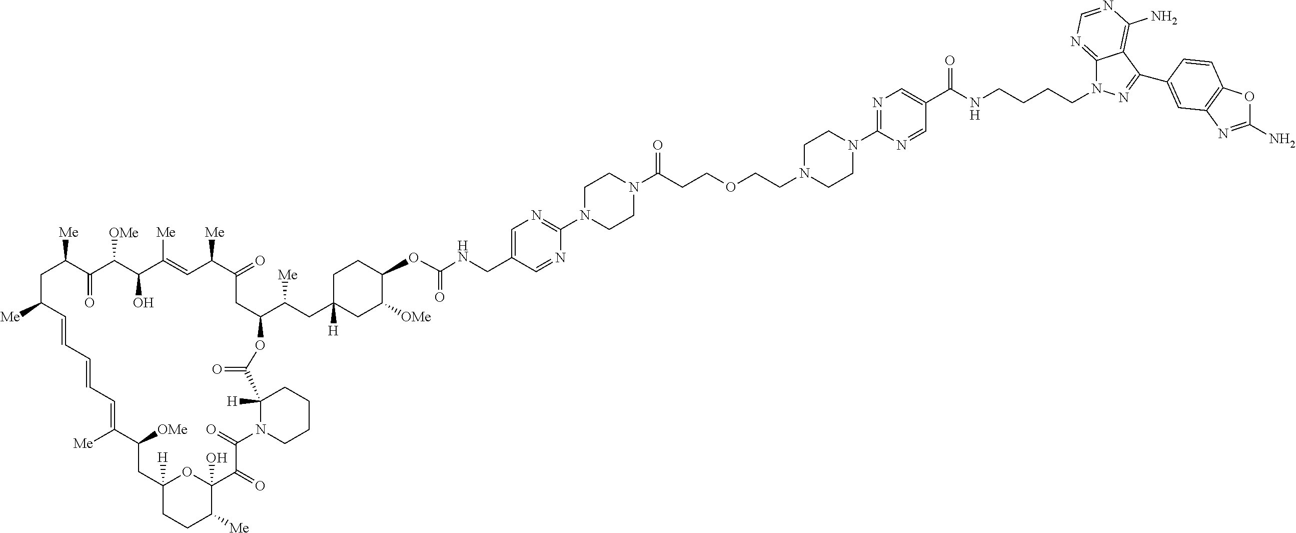 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

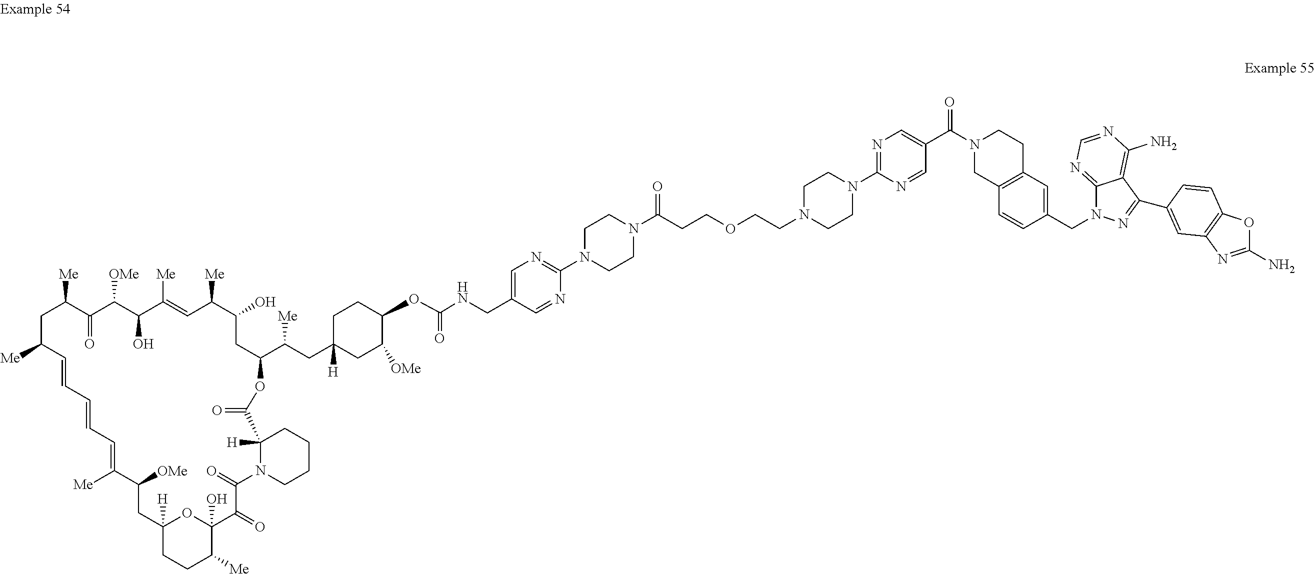 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

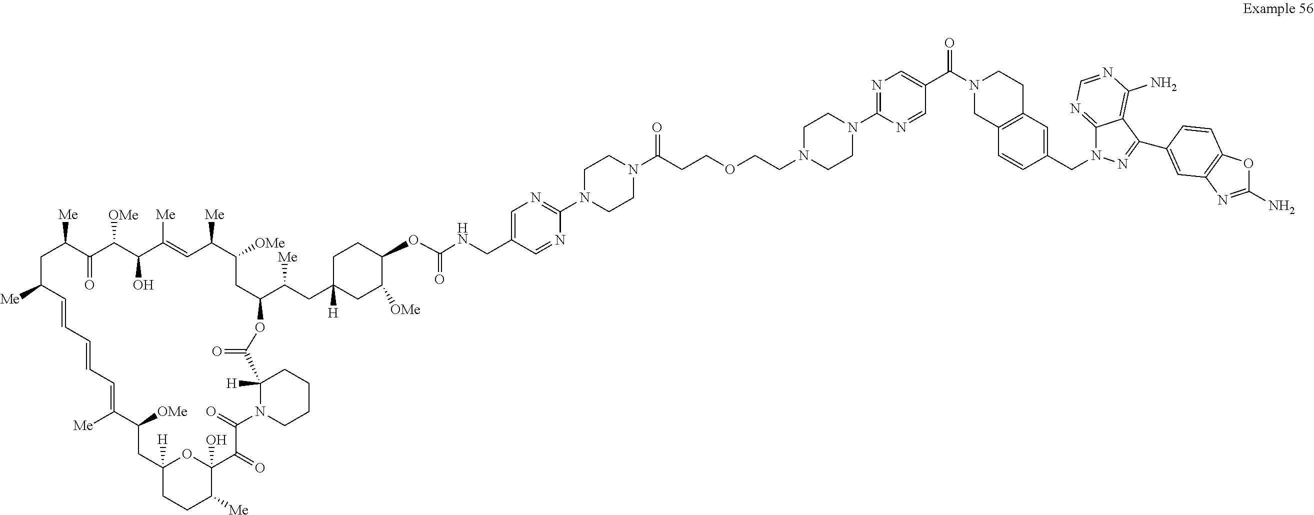 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

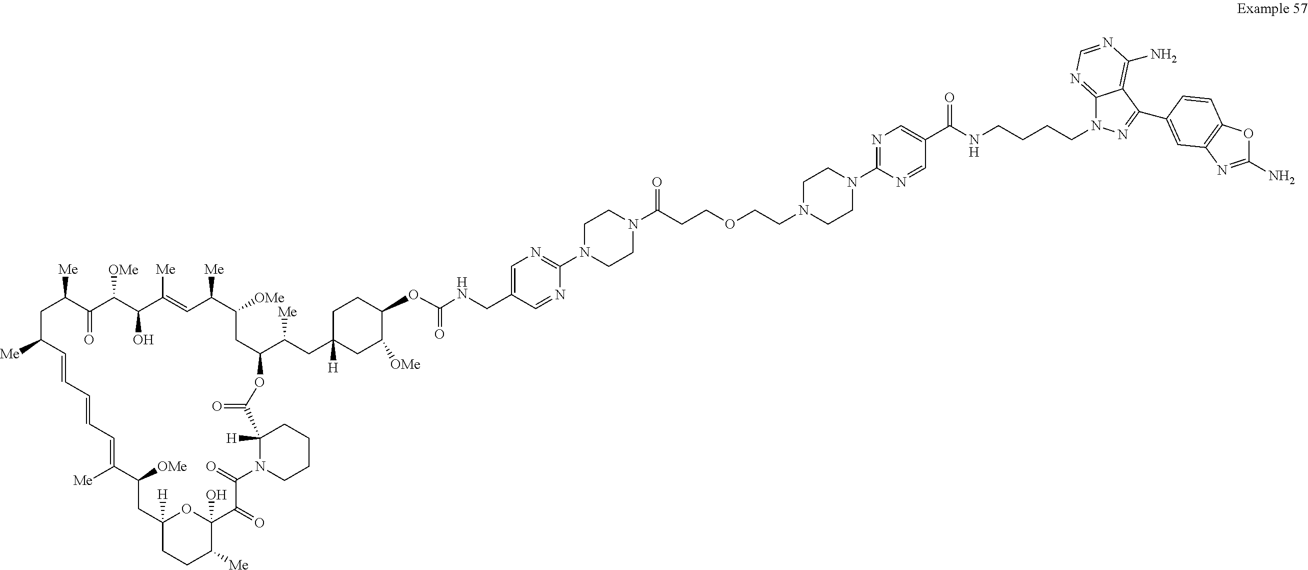 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

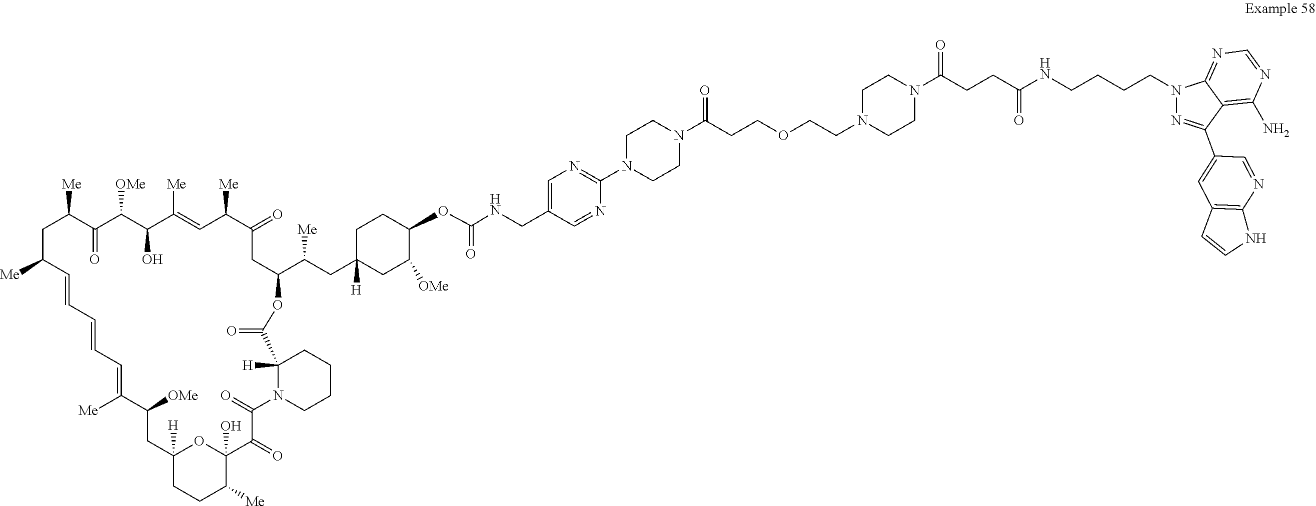 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)NCCCCn6nc(-c7cnc8[nH]ccc8c7)c7c(N)ncnc76)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

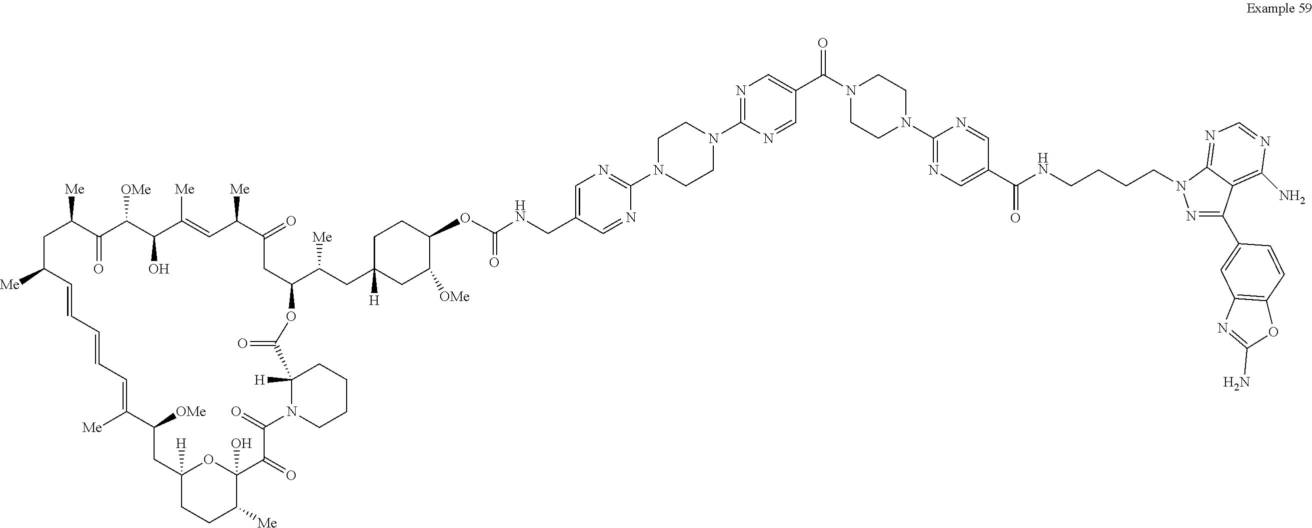 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

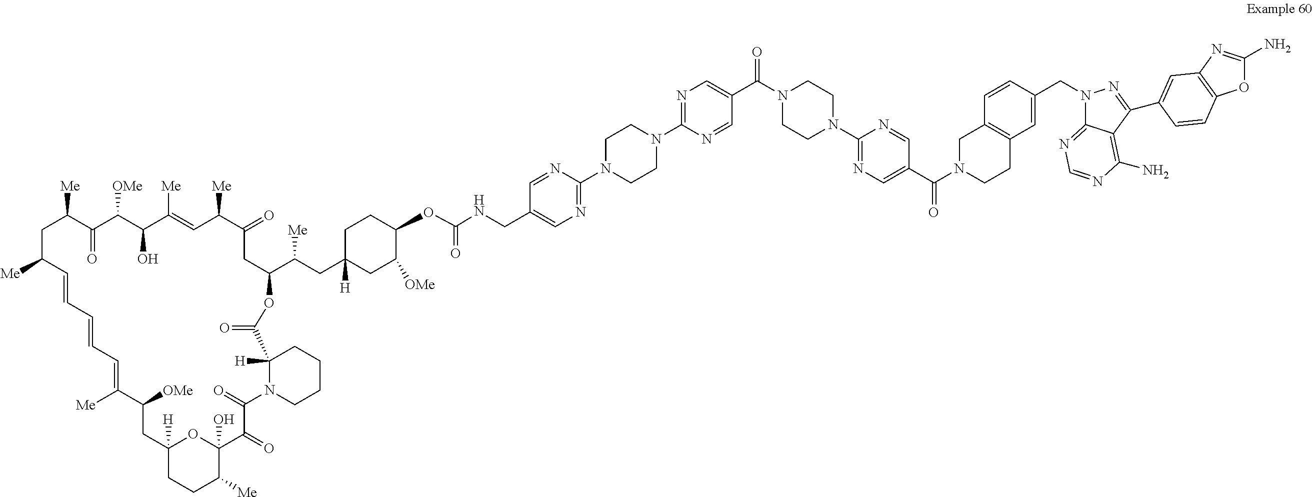 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11ccc%12oc(N)nc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

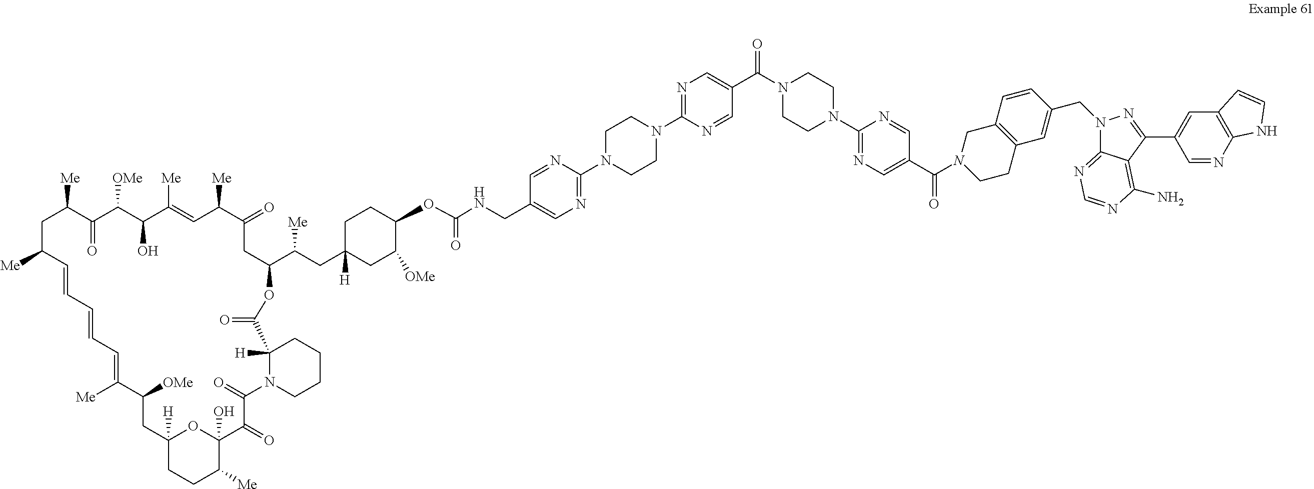 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11cnc%12[nH]ccc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

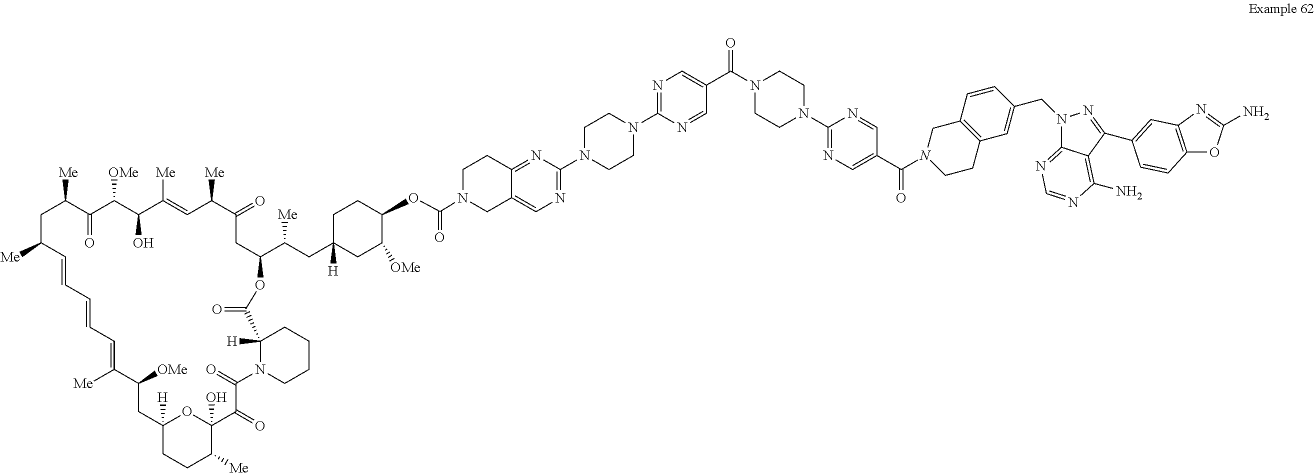 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)N7CCN(c8ncc(C(=O)N9CCc%10cc(Cn%11nc(-c%12ccc%13oc(N)nc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

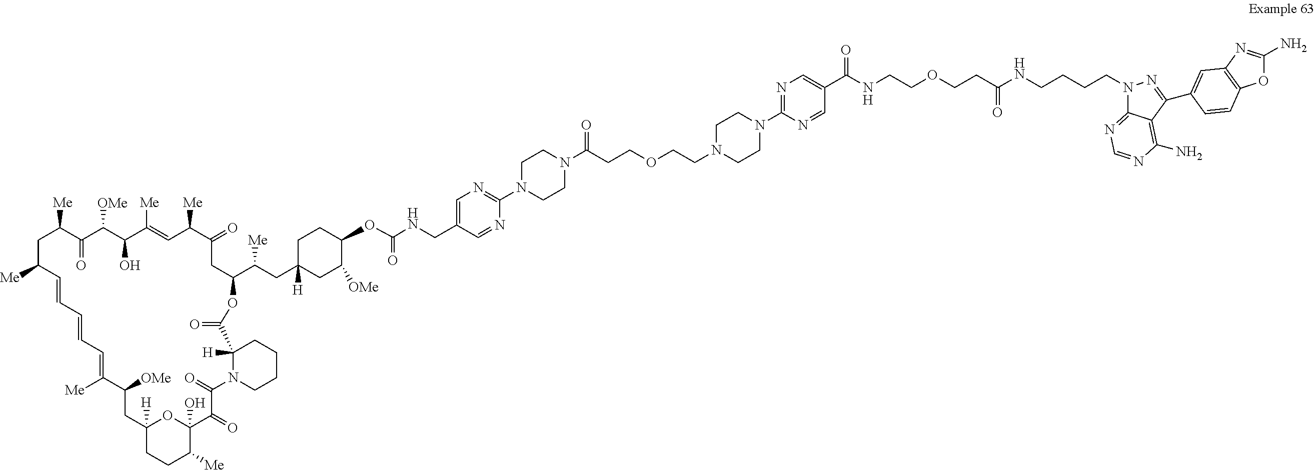 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)NCCCCn7nc(-c8ccc9oc(N)nc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

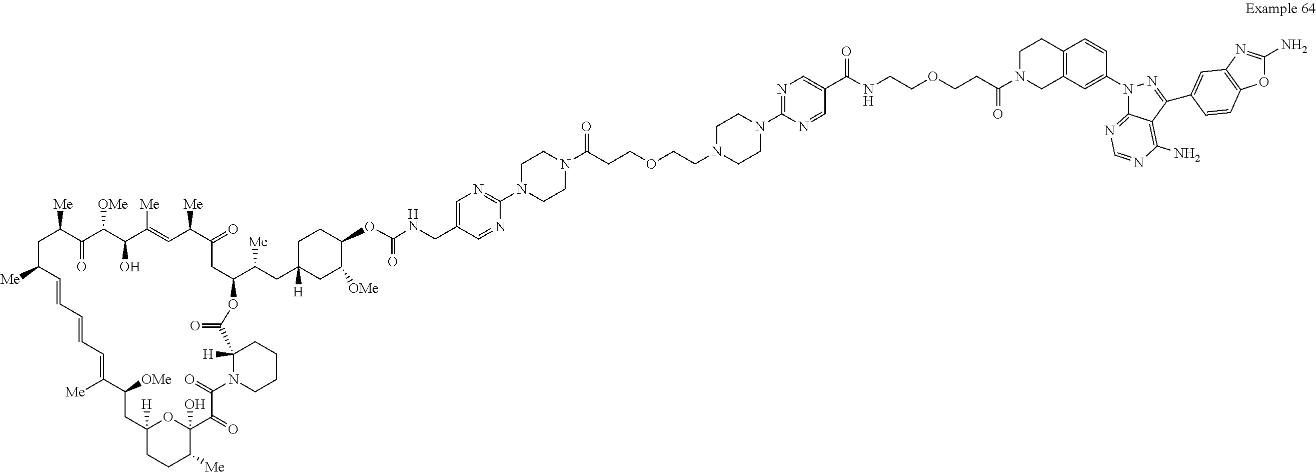 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8ccc(-n9nc(-c%10ccc%11oc(N)nc%11c%10)c%10c(N)ncnc%109)cc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

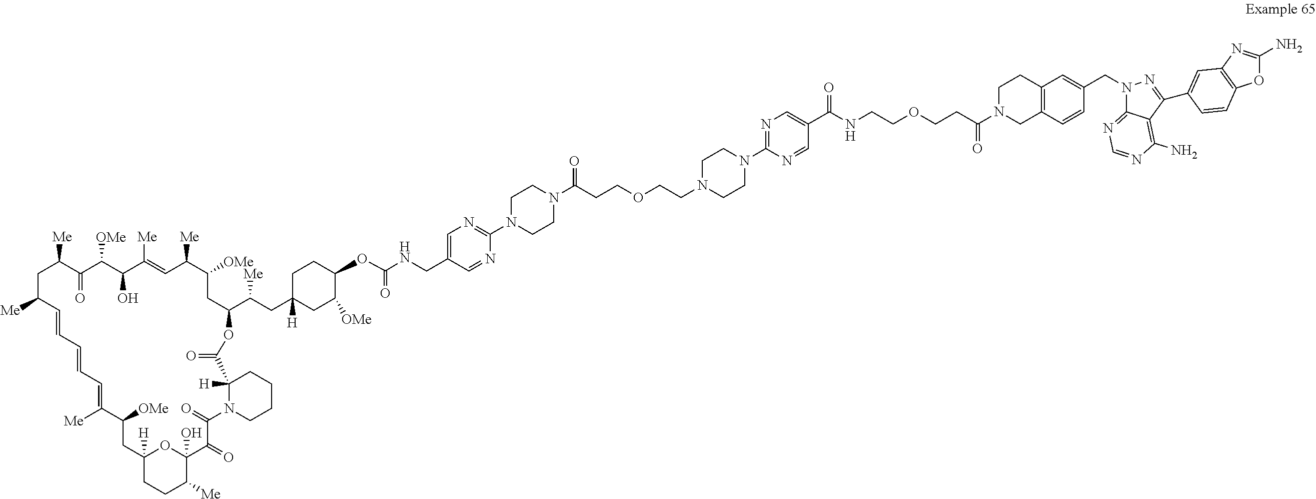 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

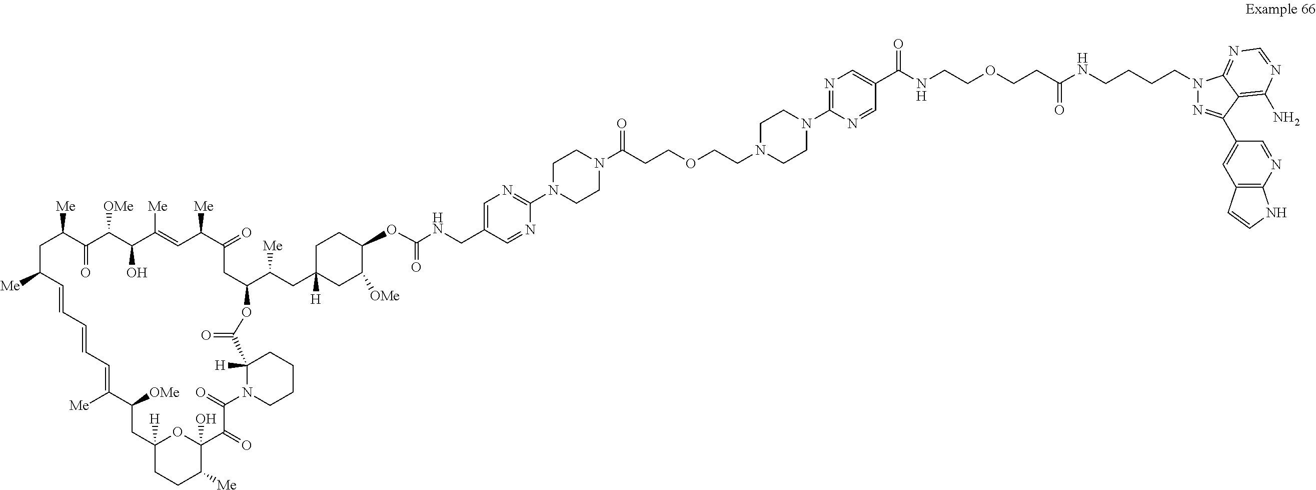 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)NCCCCn7nc(-c8cnc9[nH]ccc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

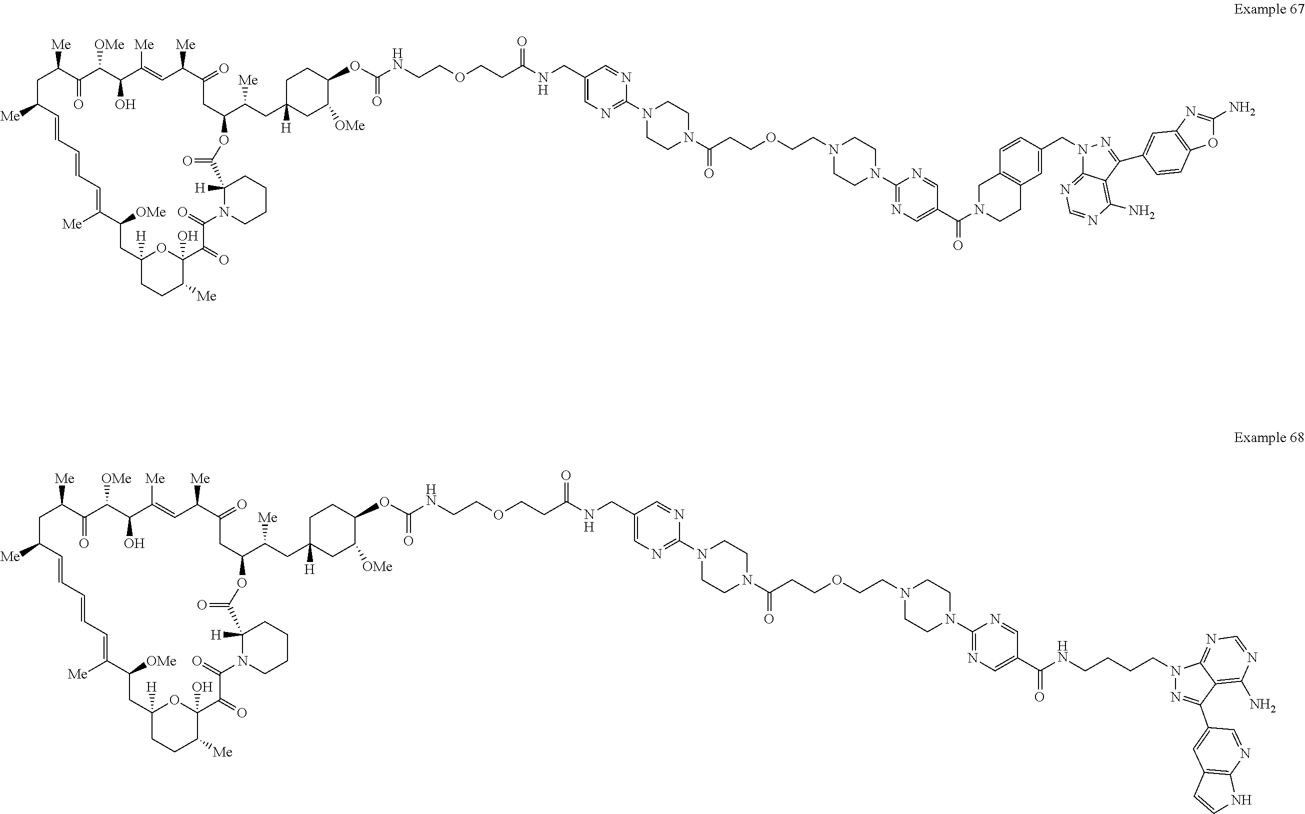

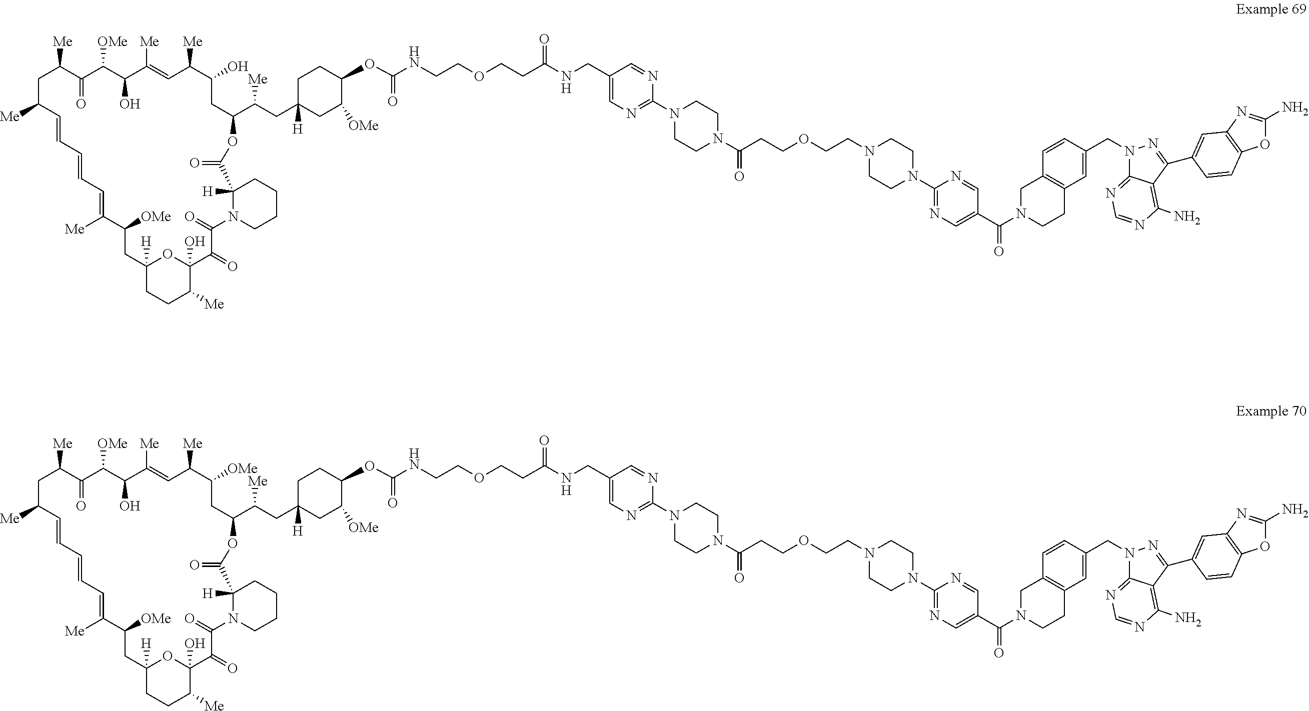

In certain embodiments, the present disclosure provides for a compound selected from below or a pharmaceutically acceptable salt or tautomer thereof,

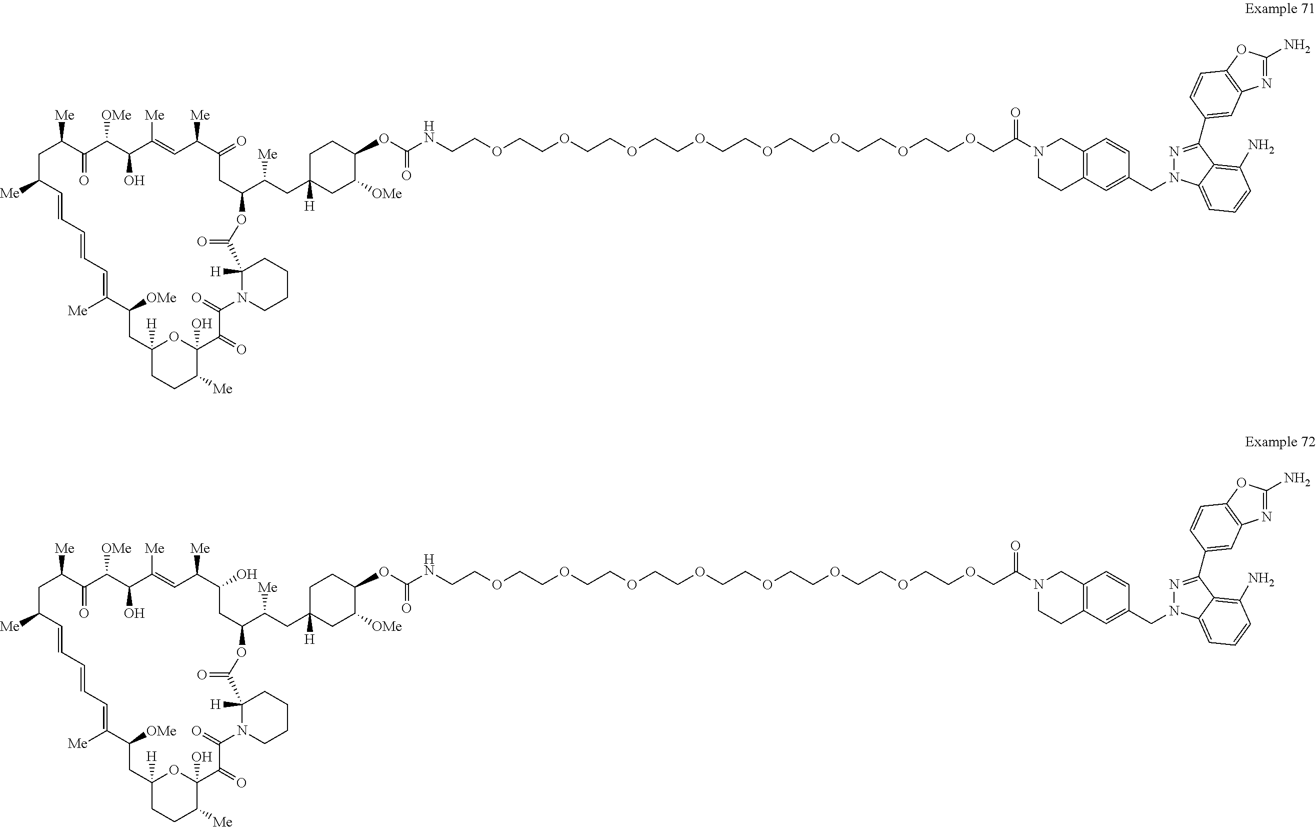

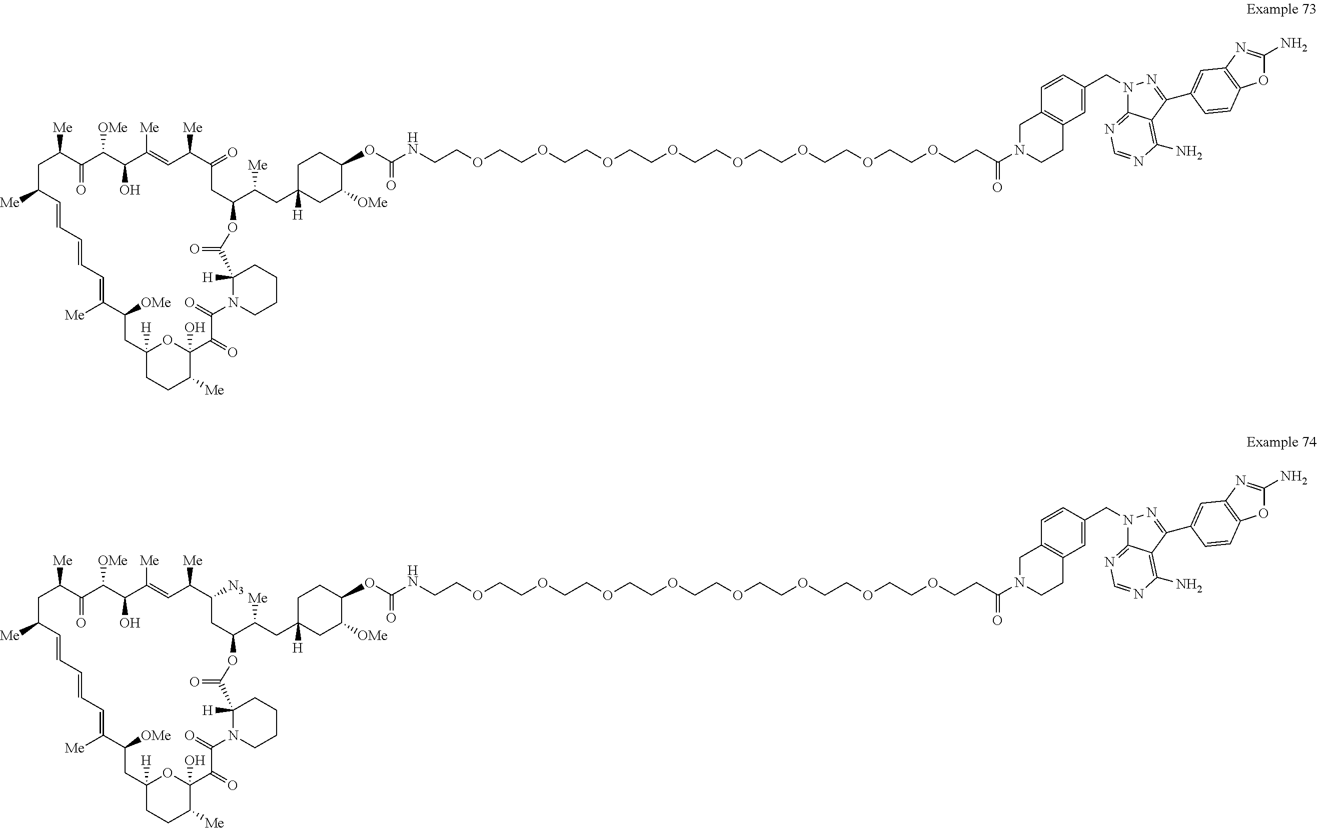

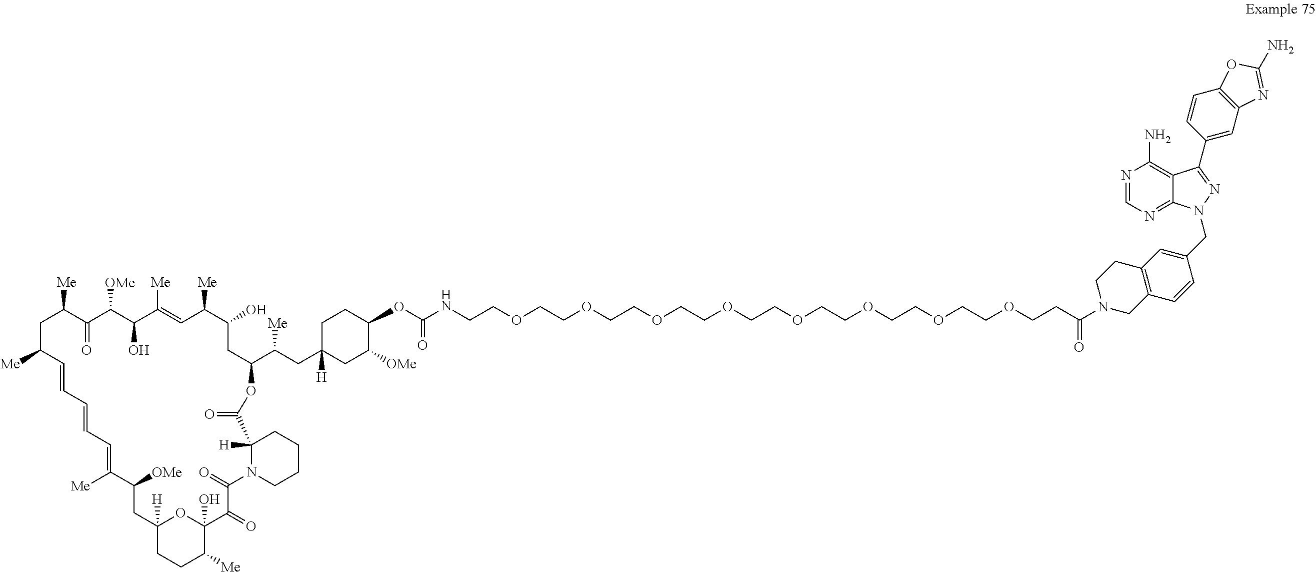 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

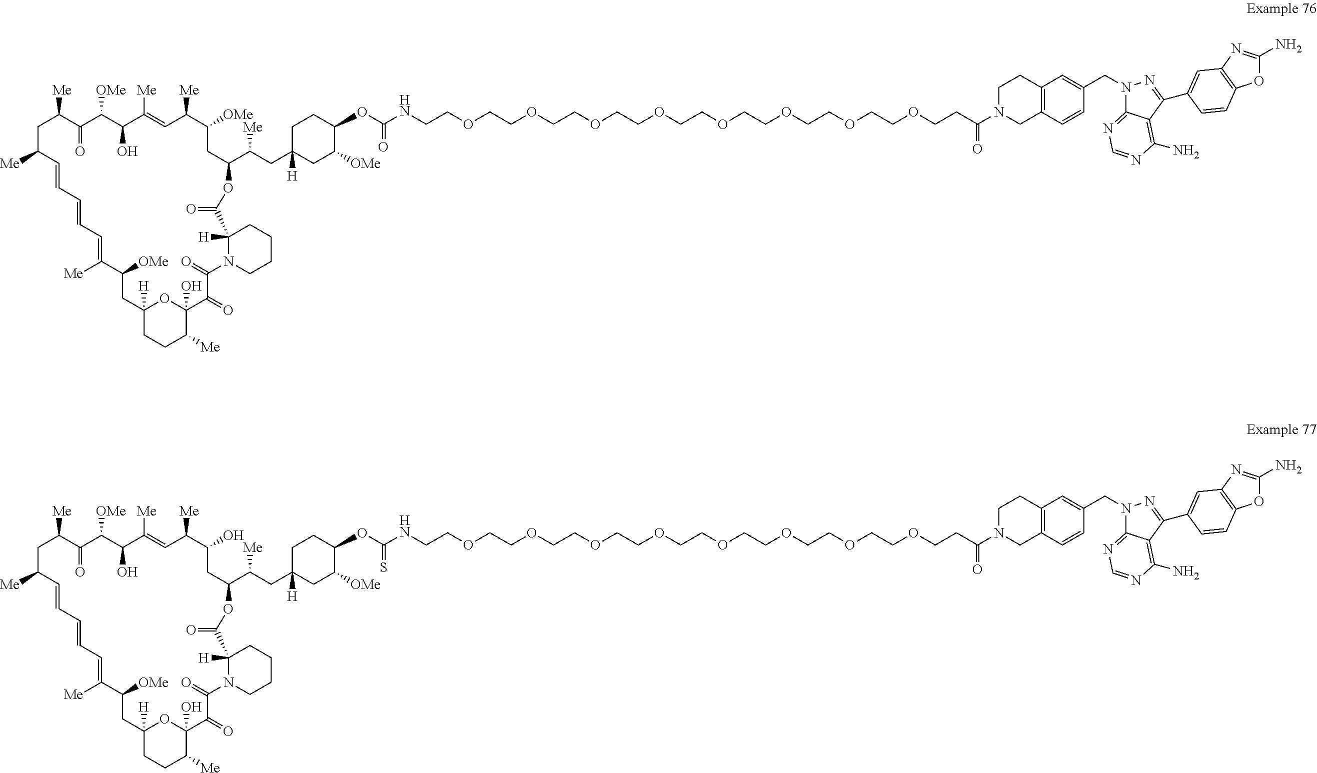

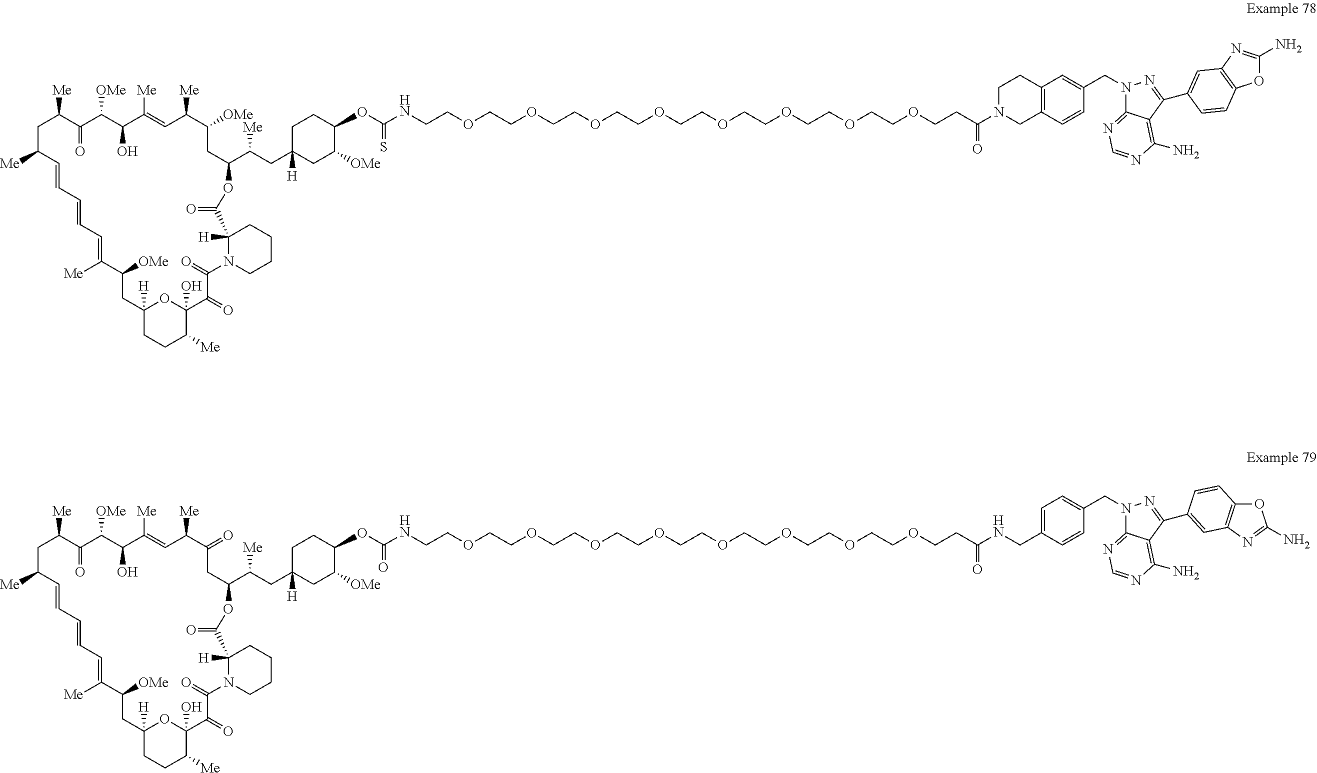

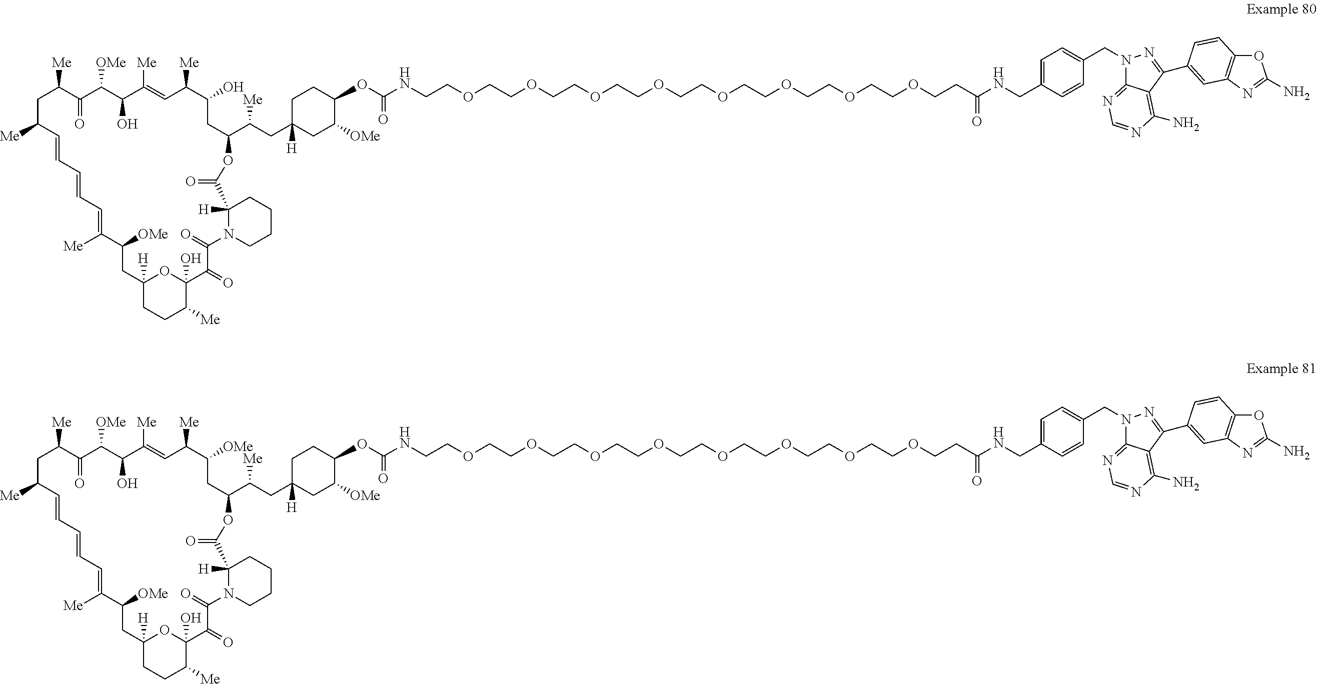

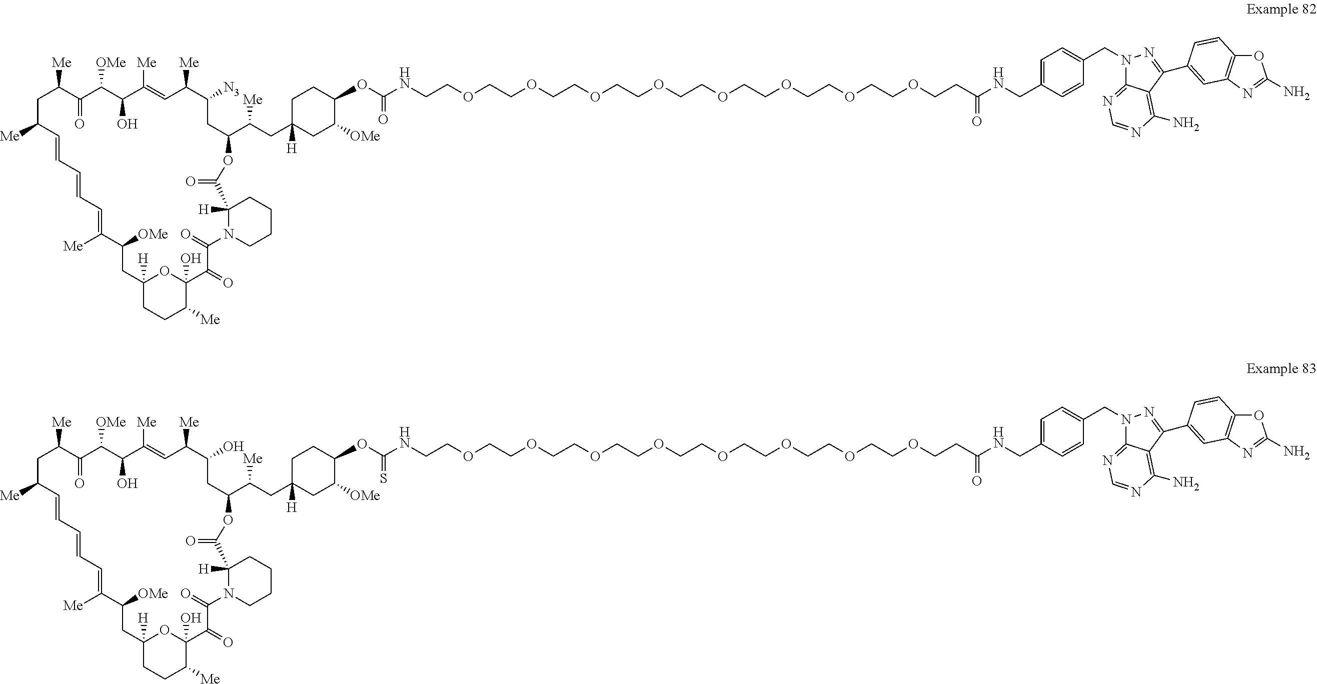

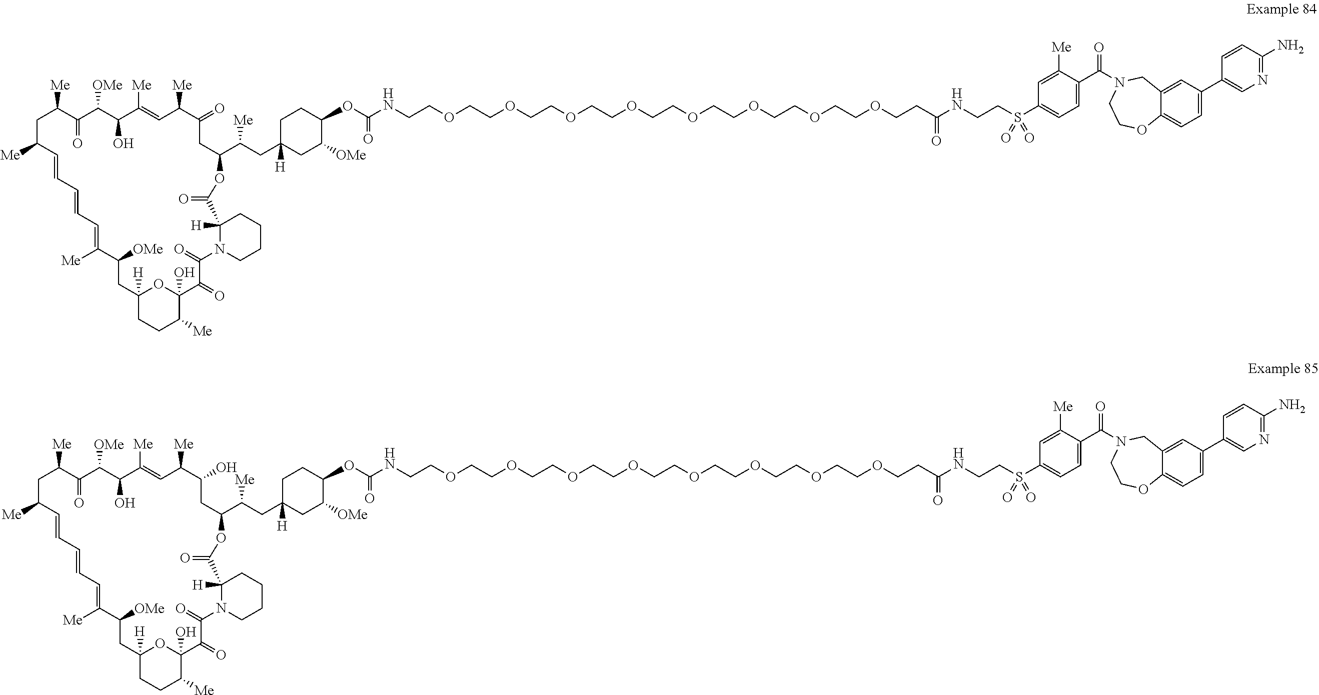

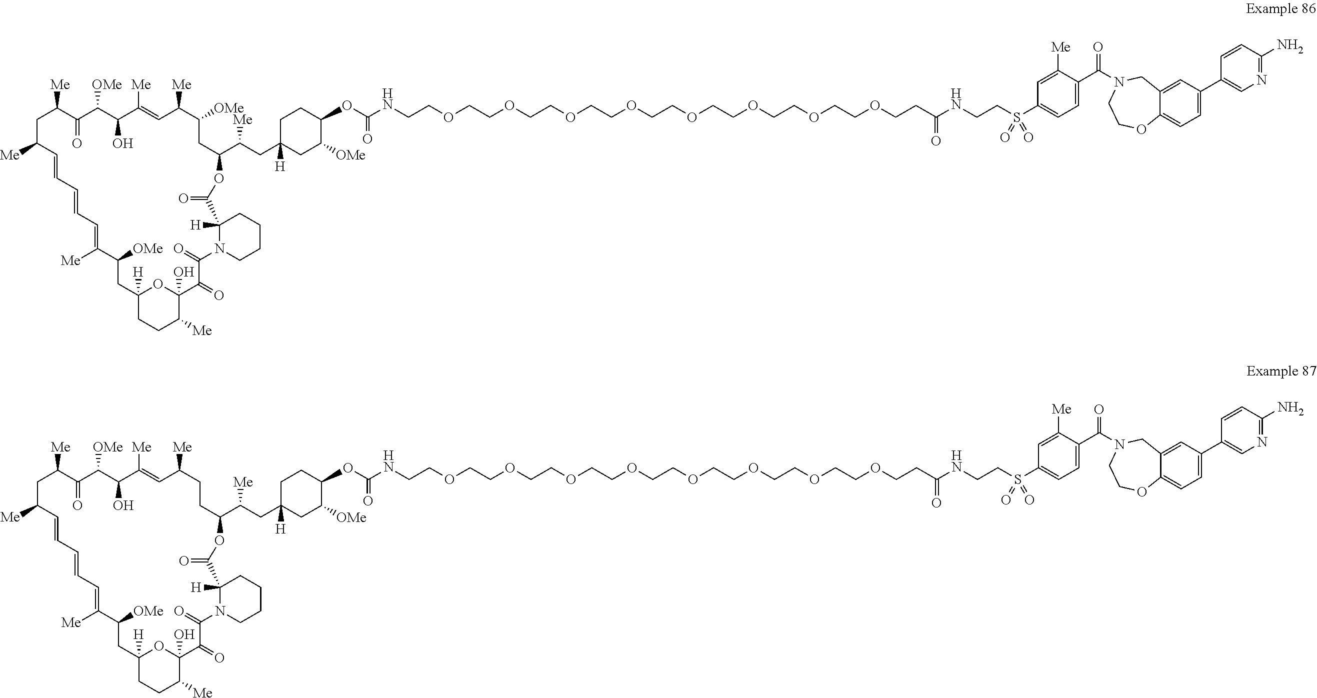

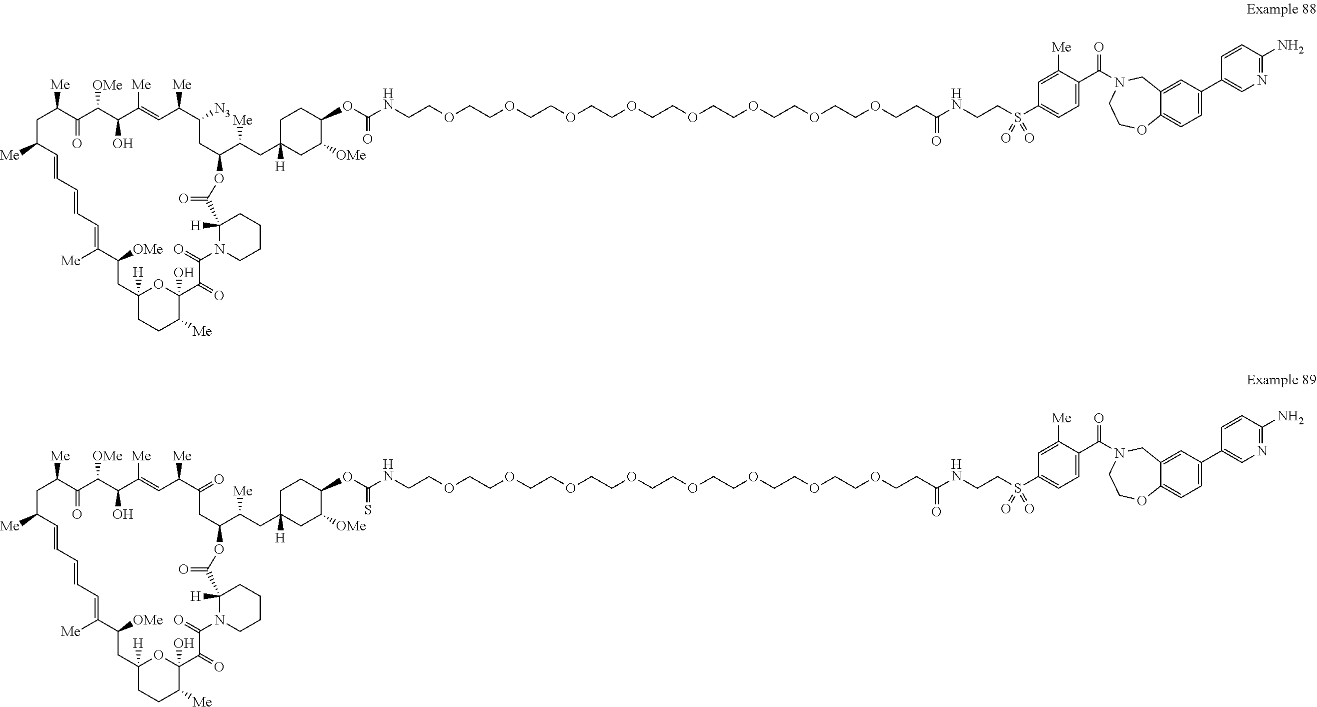

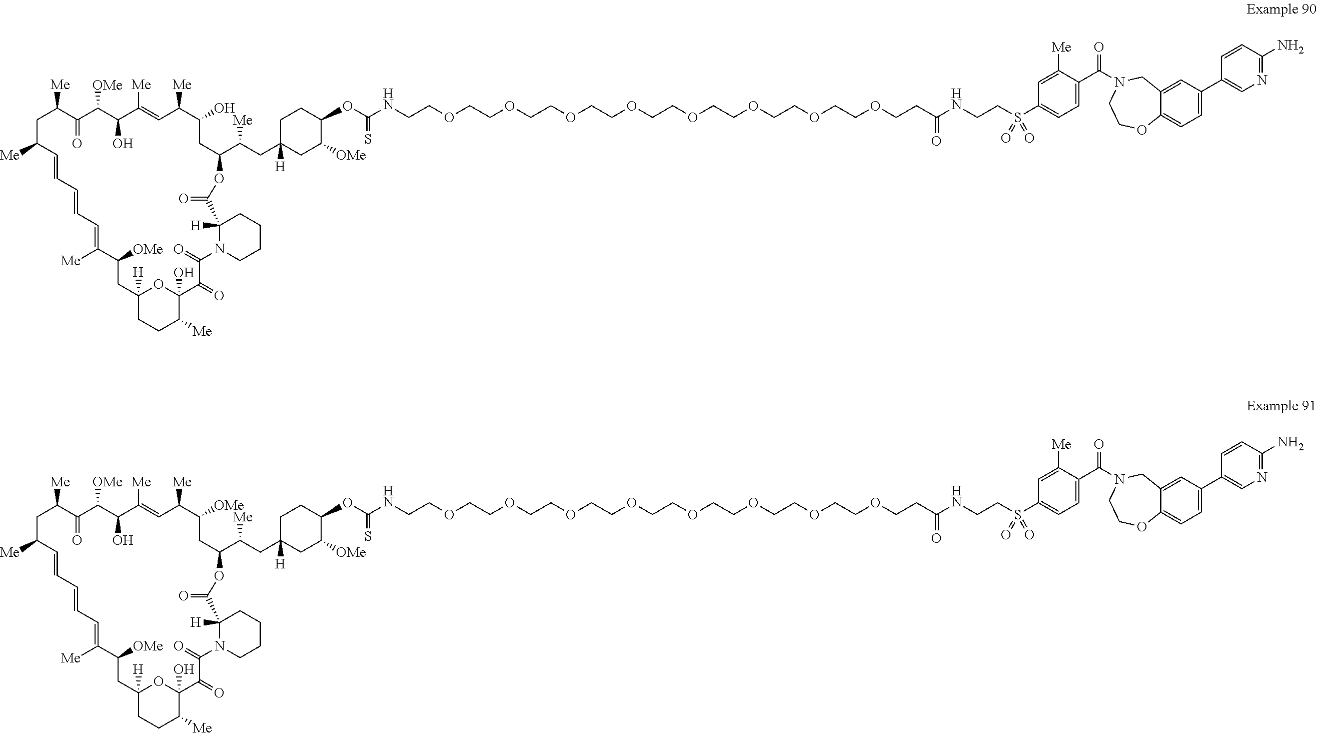

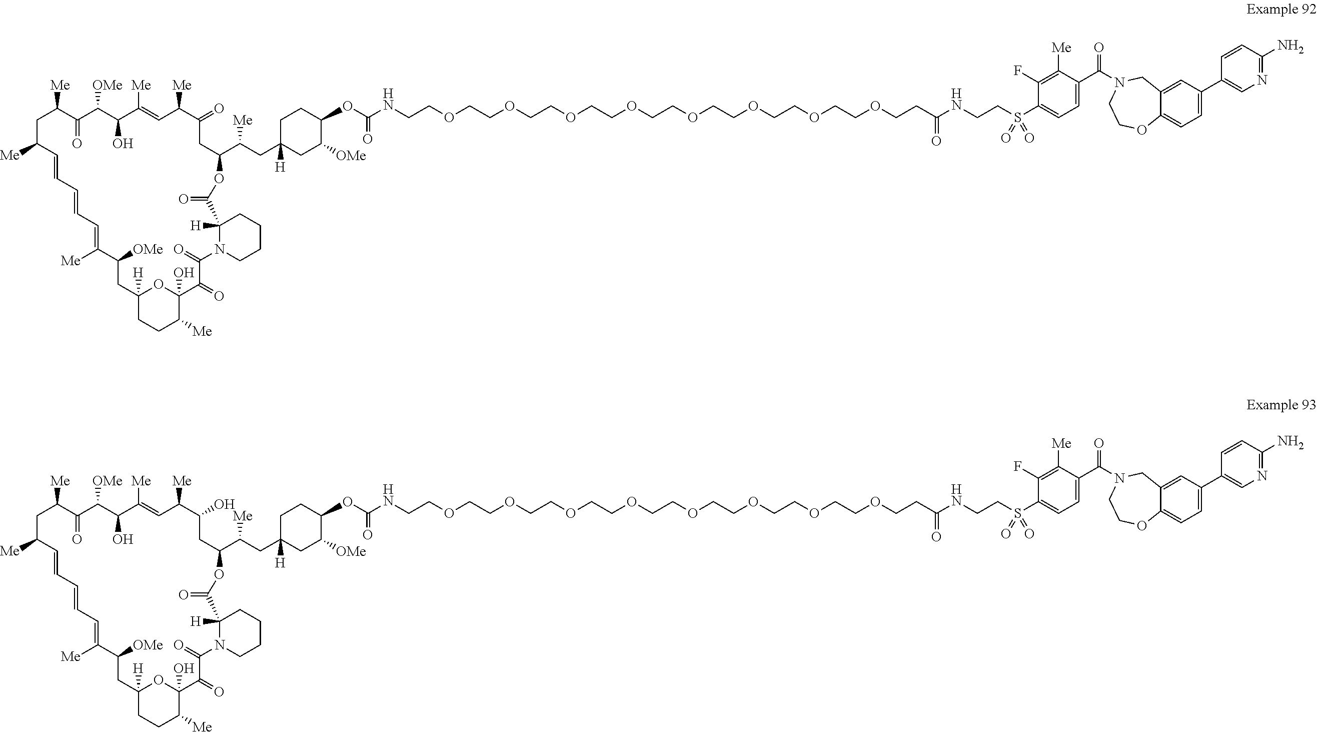

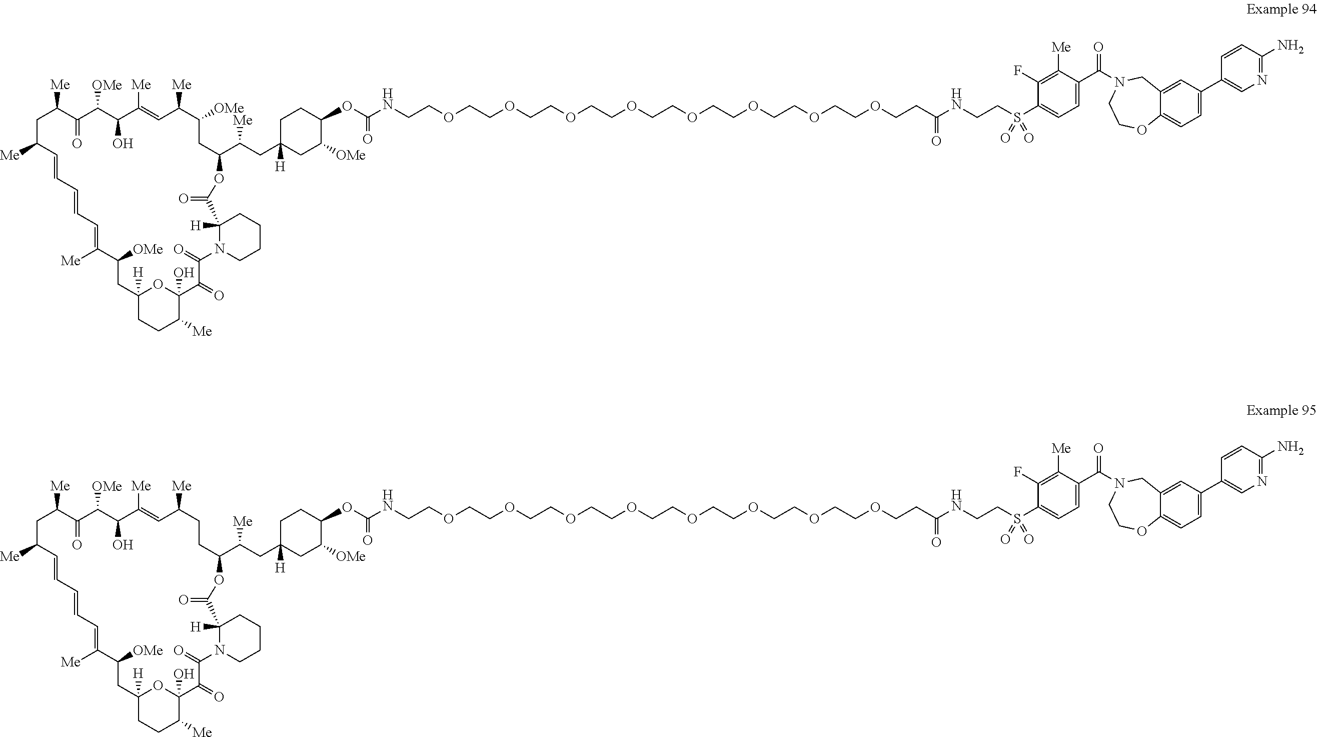

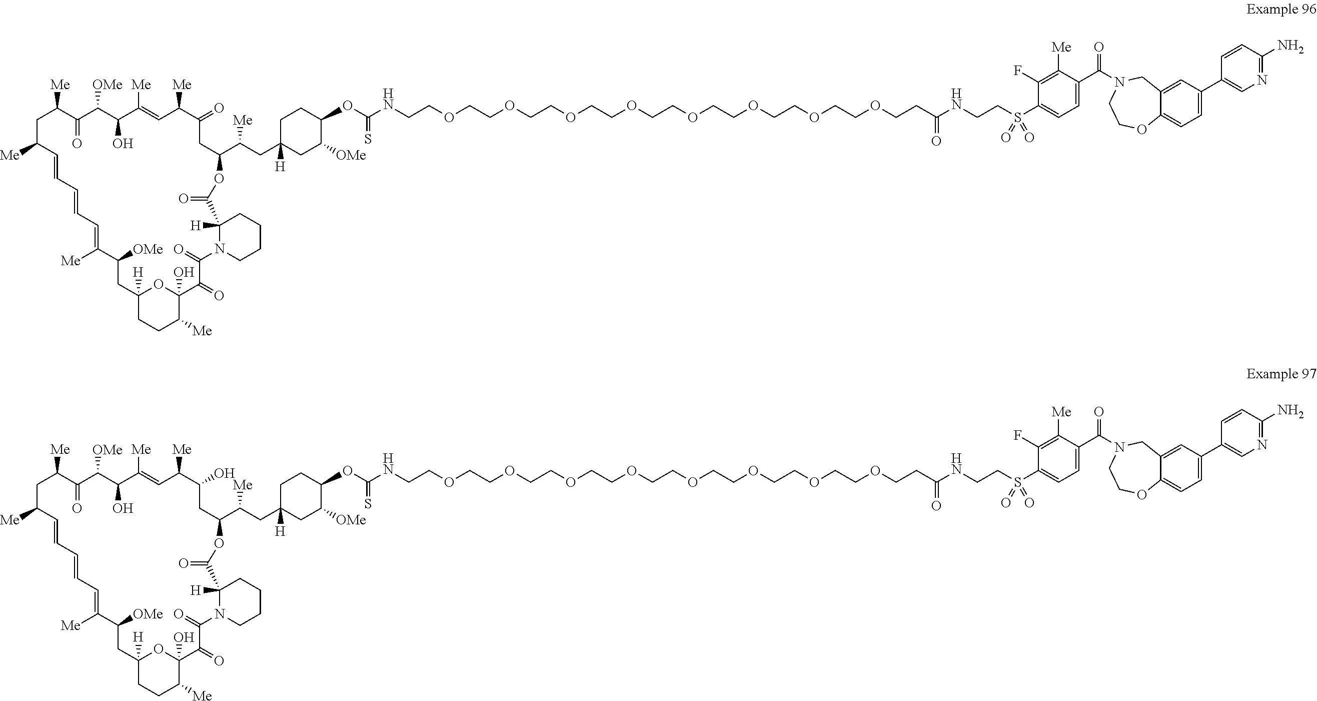

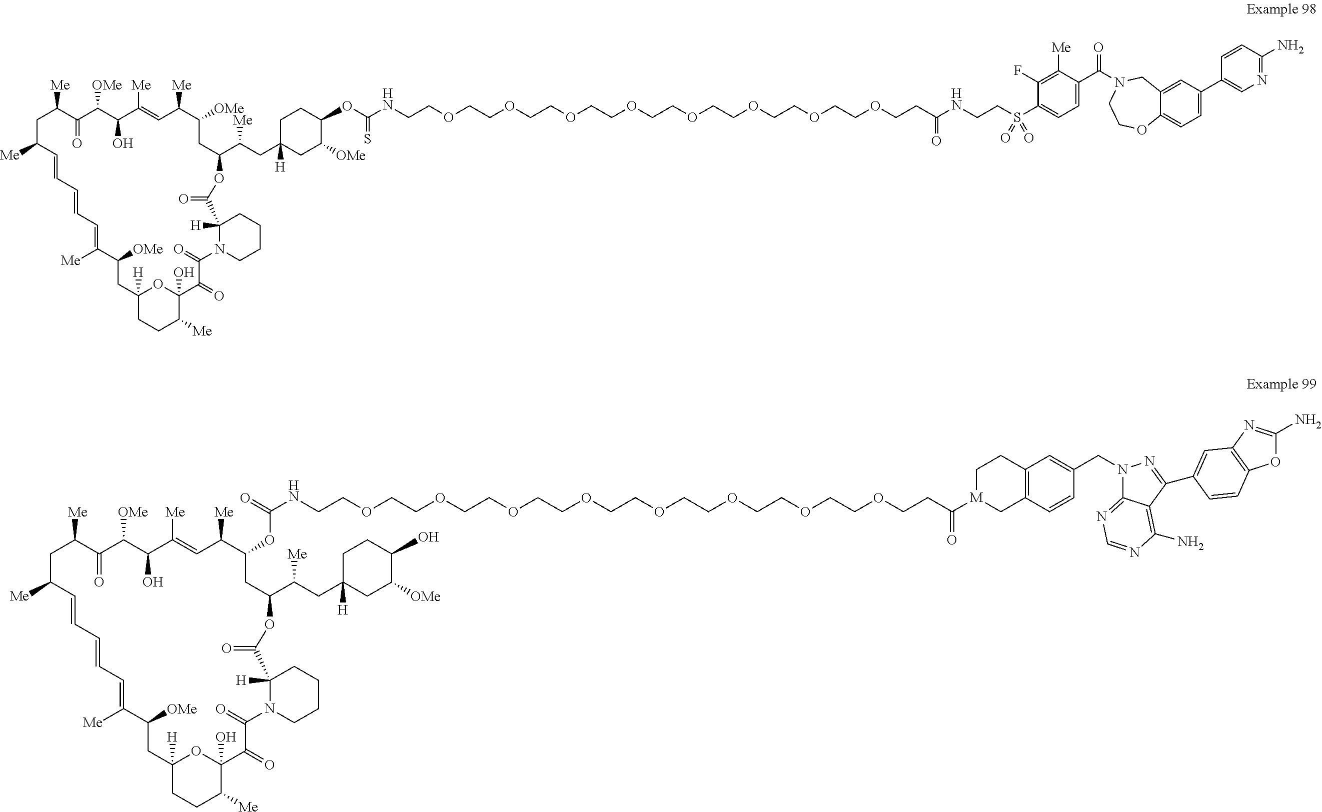

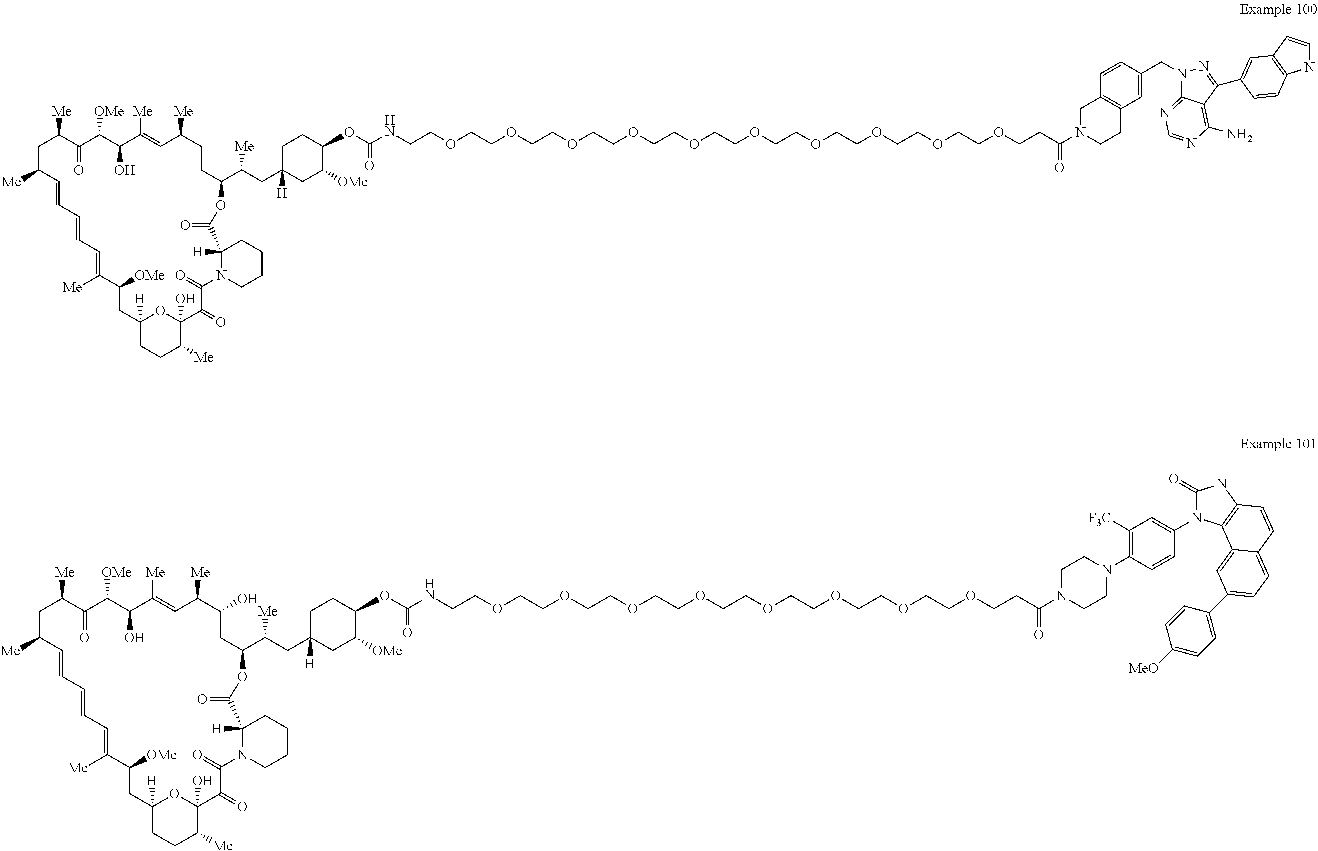

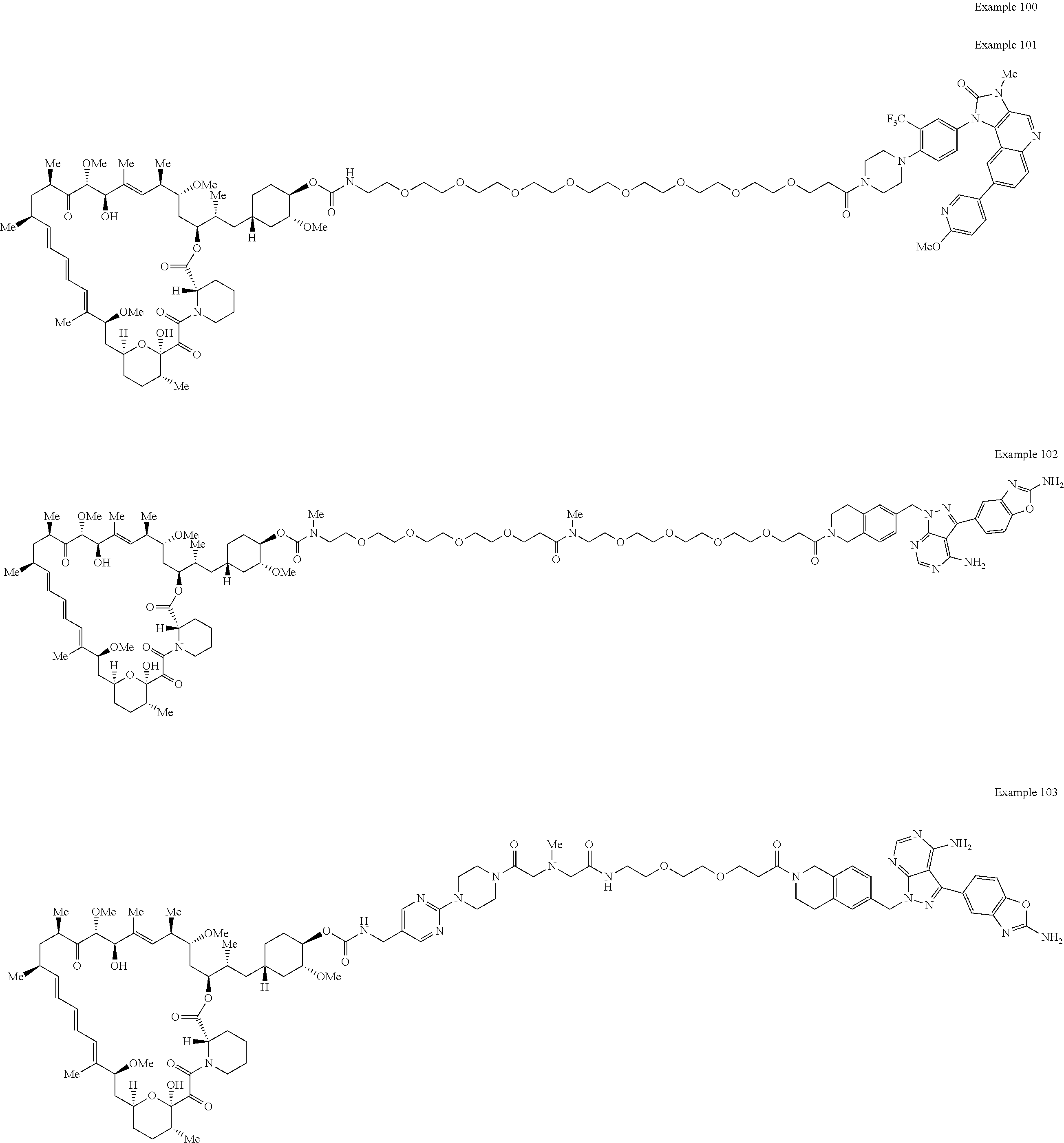

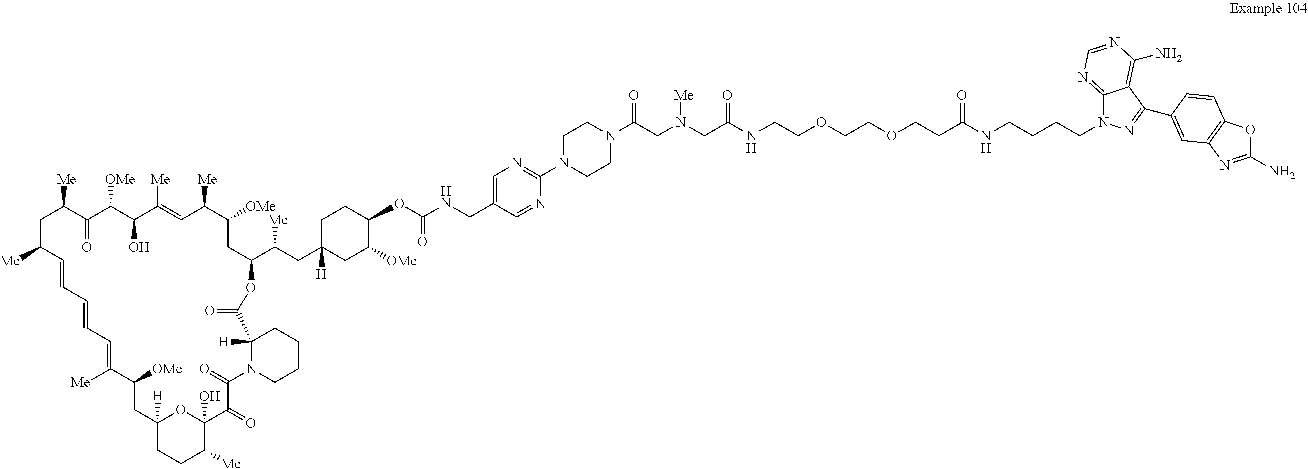 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CN(C)CC(=O)NCCOCCOCCC(=O)NCCCCn5nc(-c6ccc7oc(N)nc7c6)c6c(N)ncnc65)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

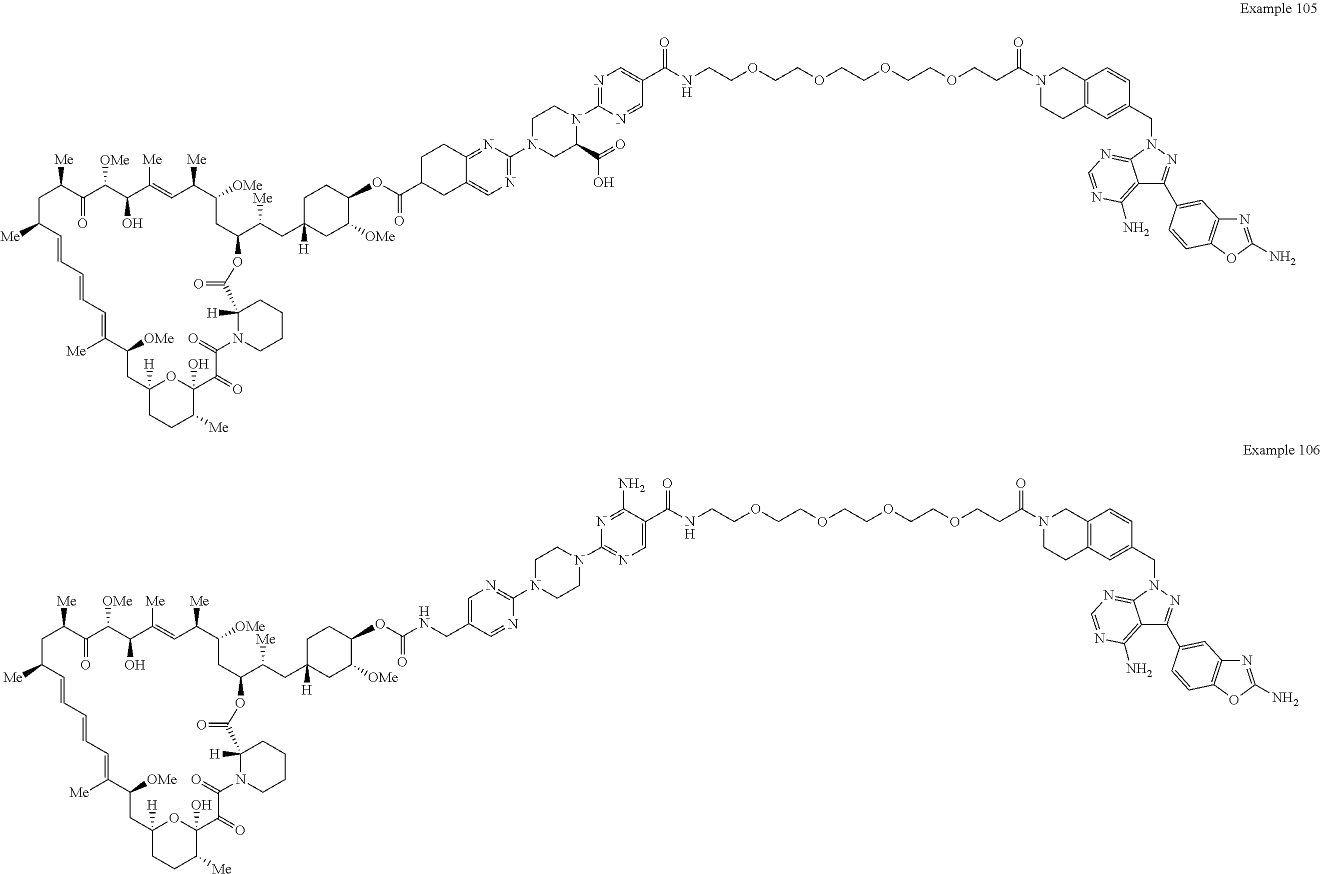

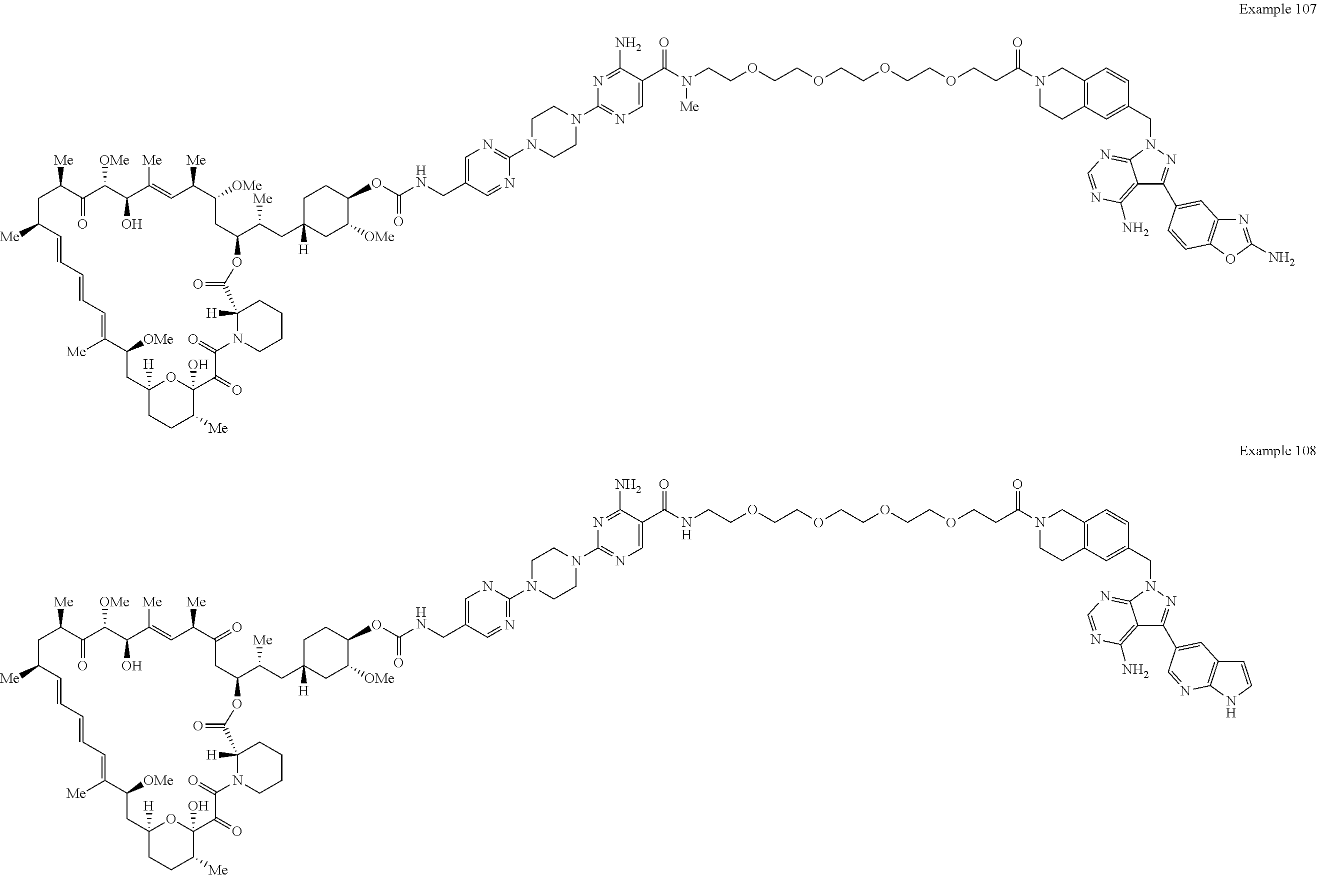

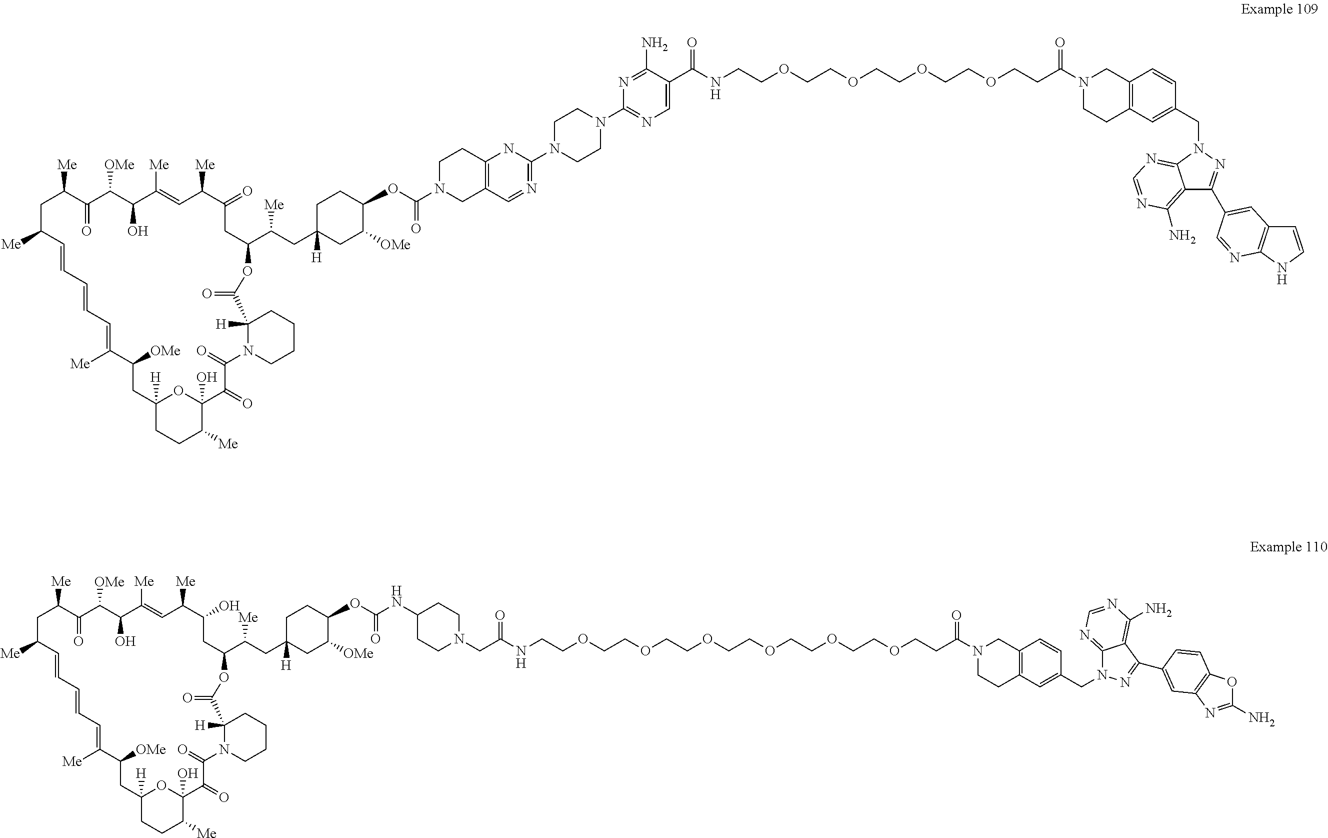

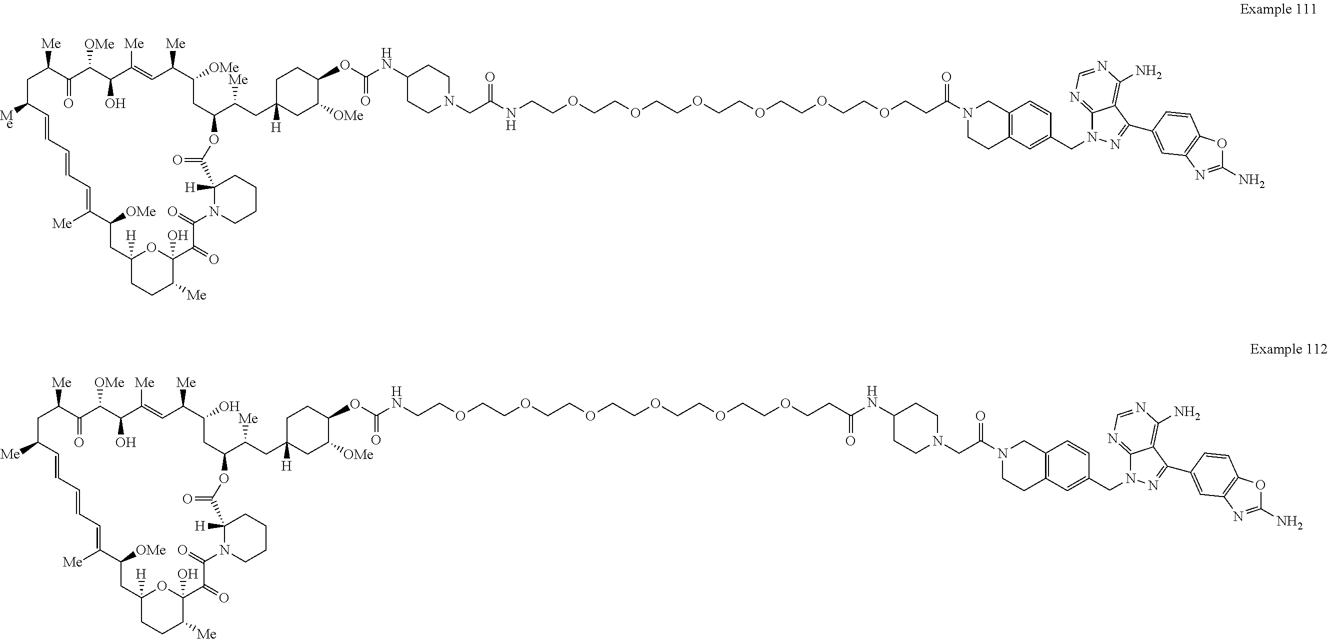

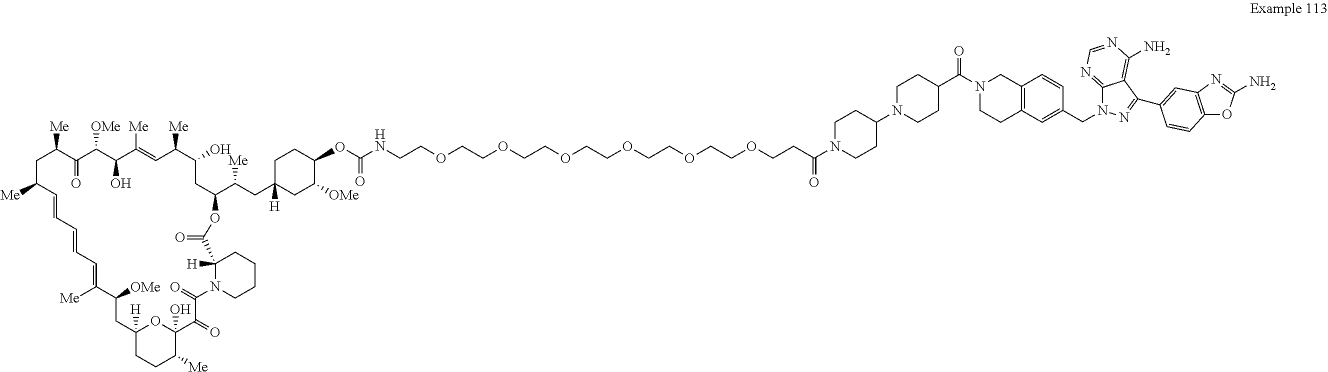 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCC(=O)N3CCC(N4CCC(C(=O)N5CCc6cc(Cn7nc(-c8ccc9oc(N)nc9c8)c8c(N)ncnc87)ccc6C5)CC4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

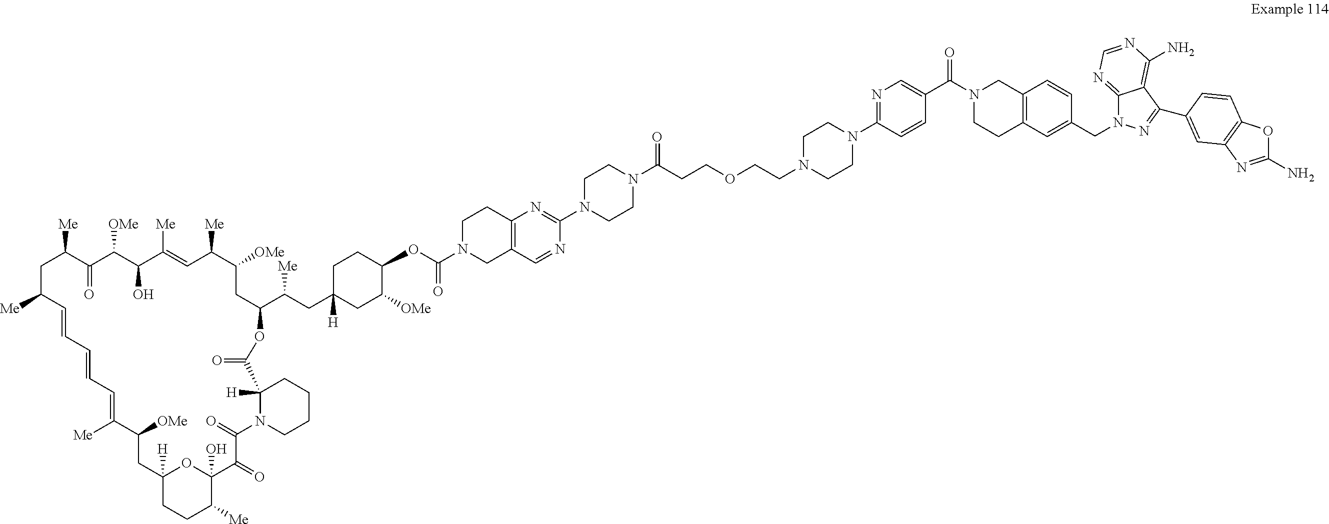 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ccc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

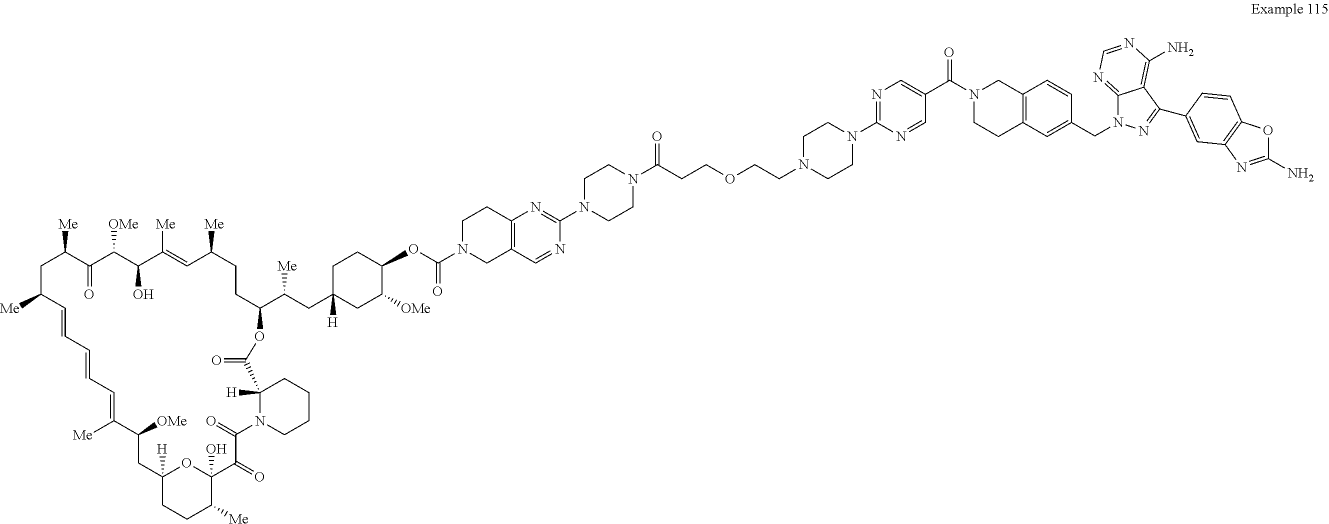 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ncc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)CC[C@H](C)/C=C(\C)[C@@H](O)[C@@H](OC)C(=O)[C@H](C)C[C@H](C)/C=C/C=C/C=C/1C]

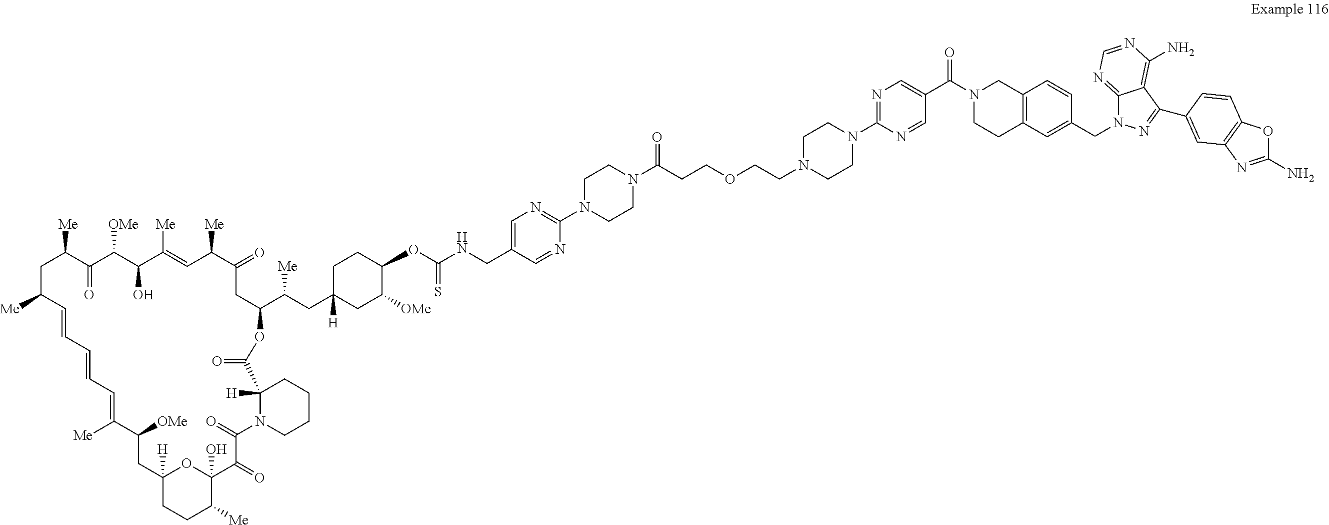 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

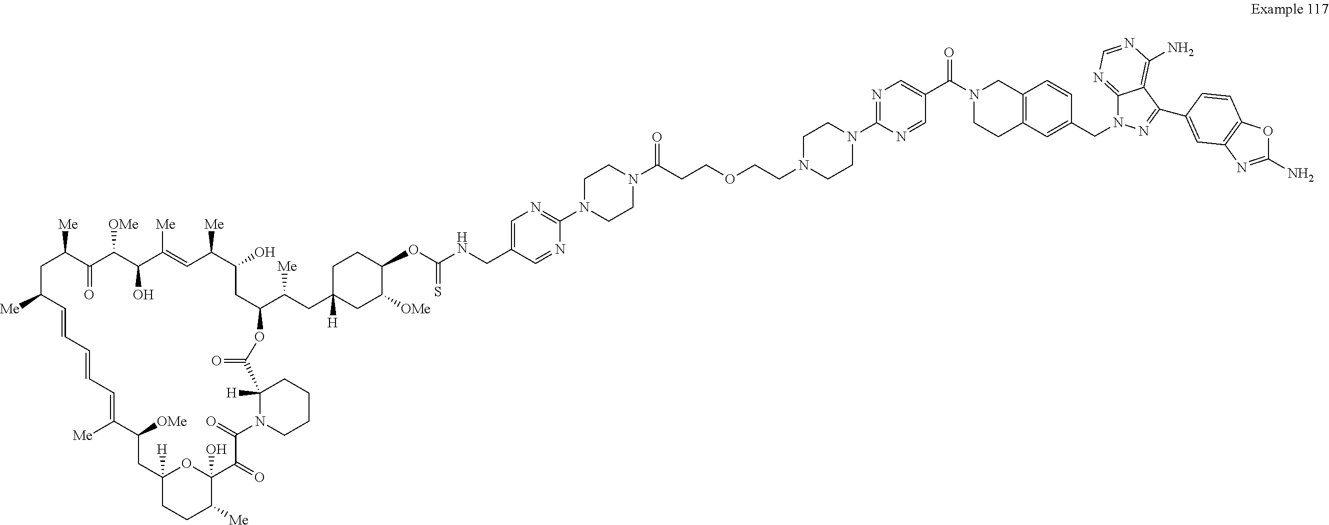 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

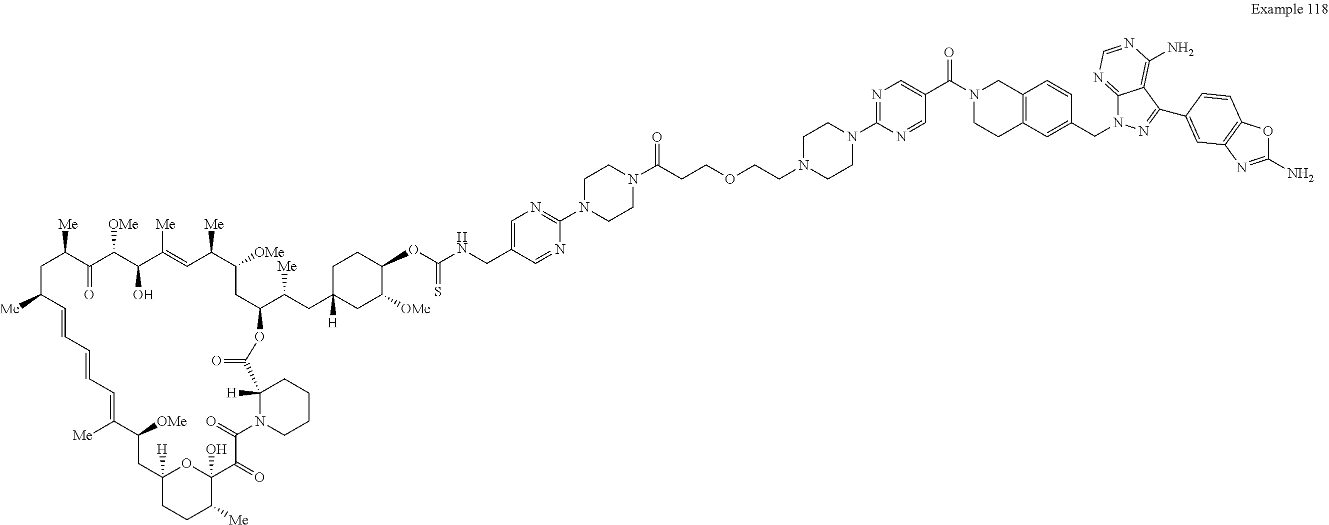 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

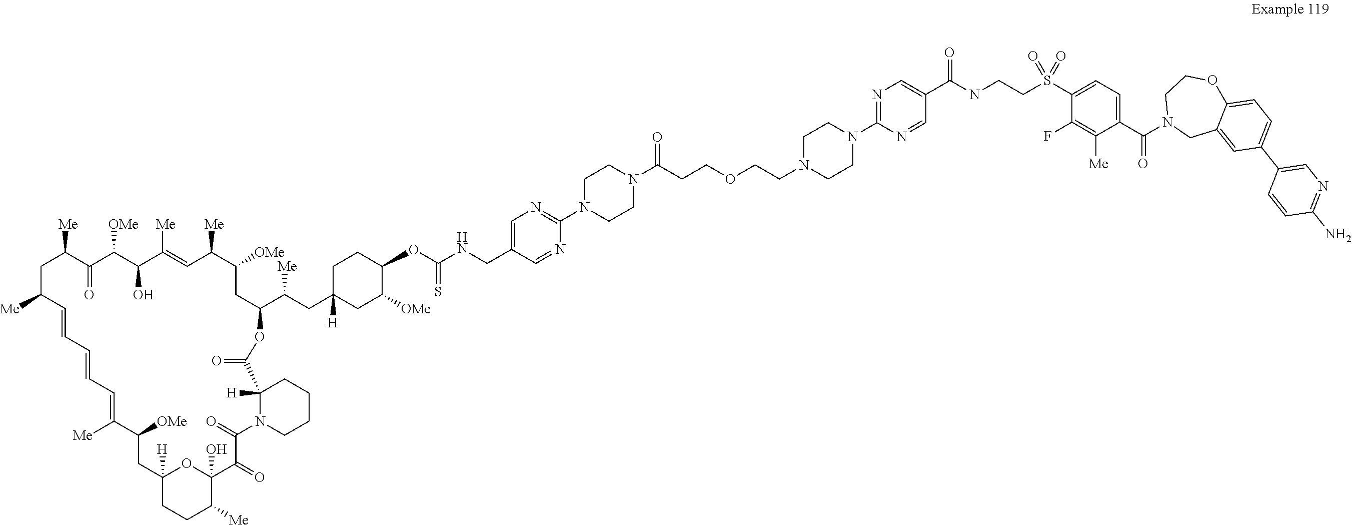 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7F)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

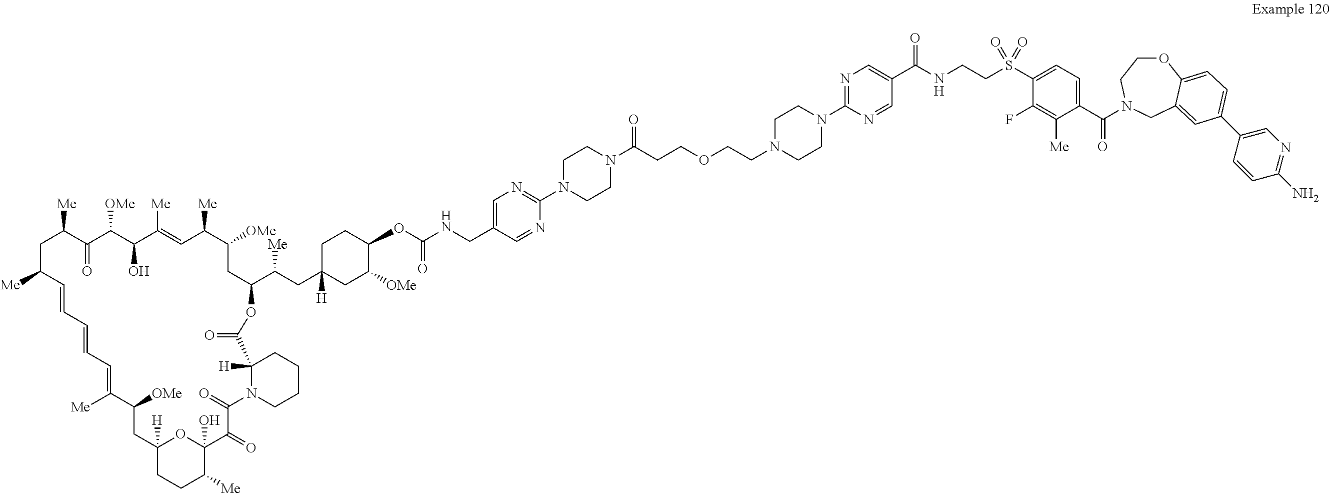 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7F)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

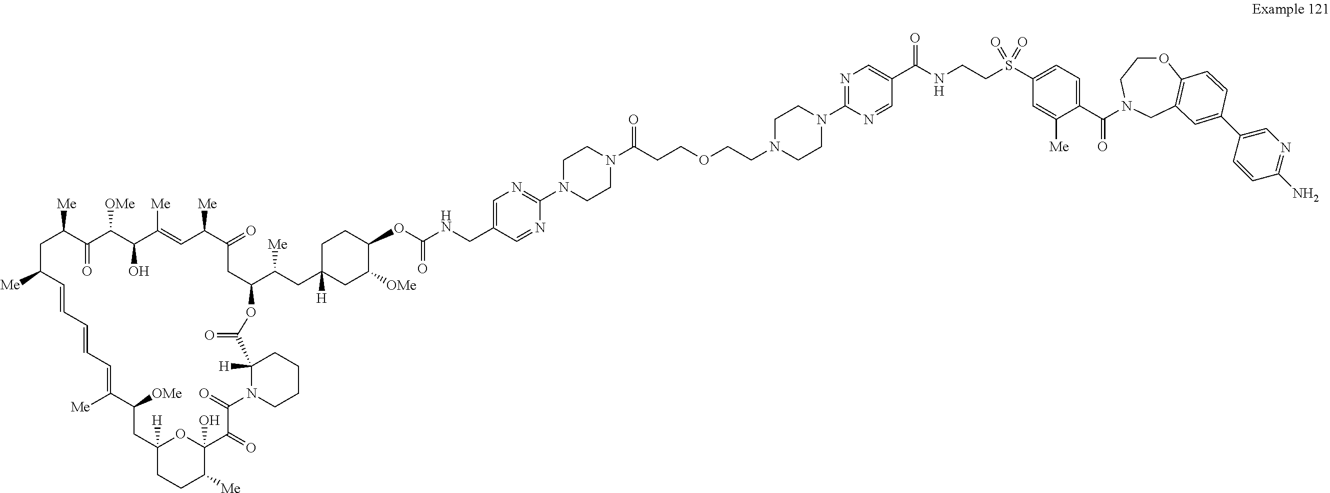 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

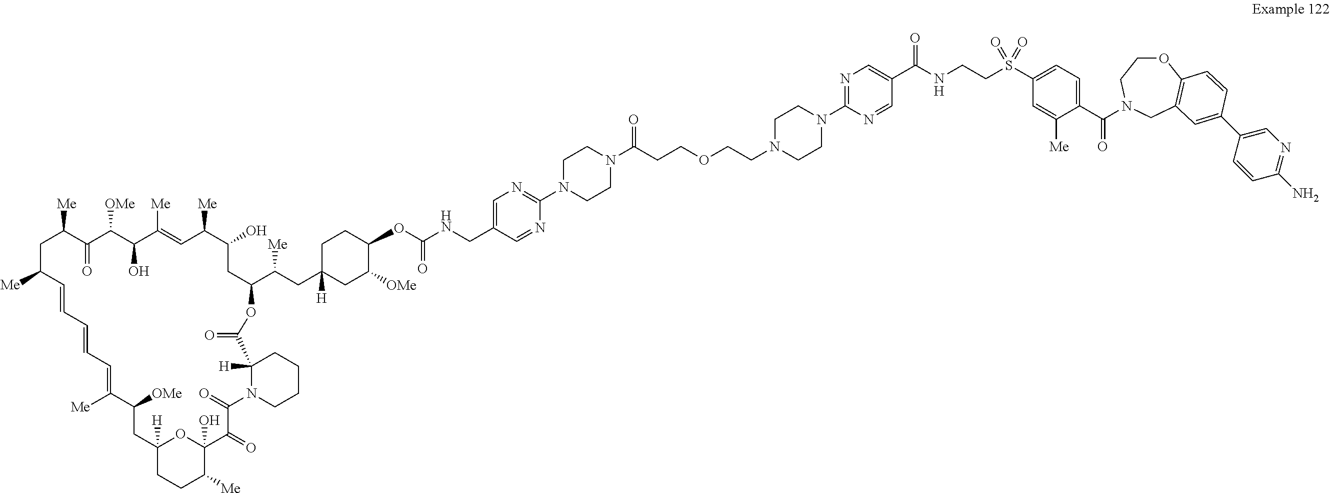 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

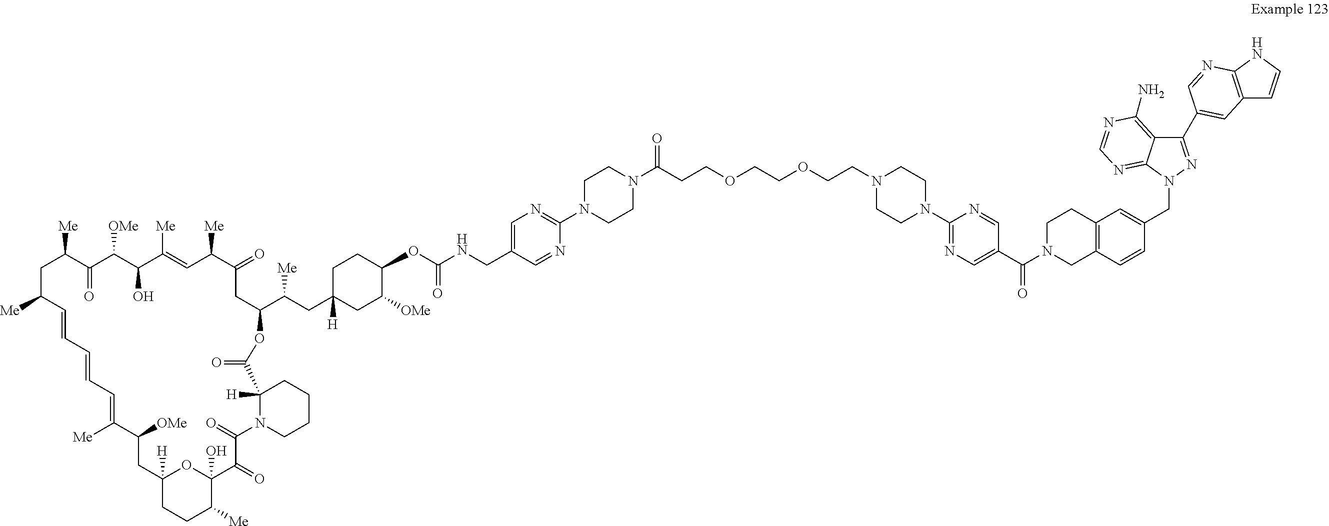 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

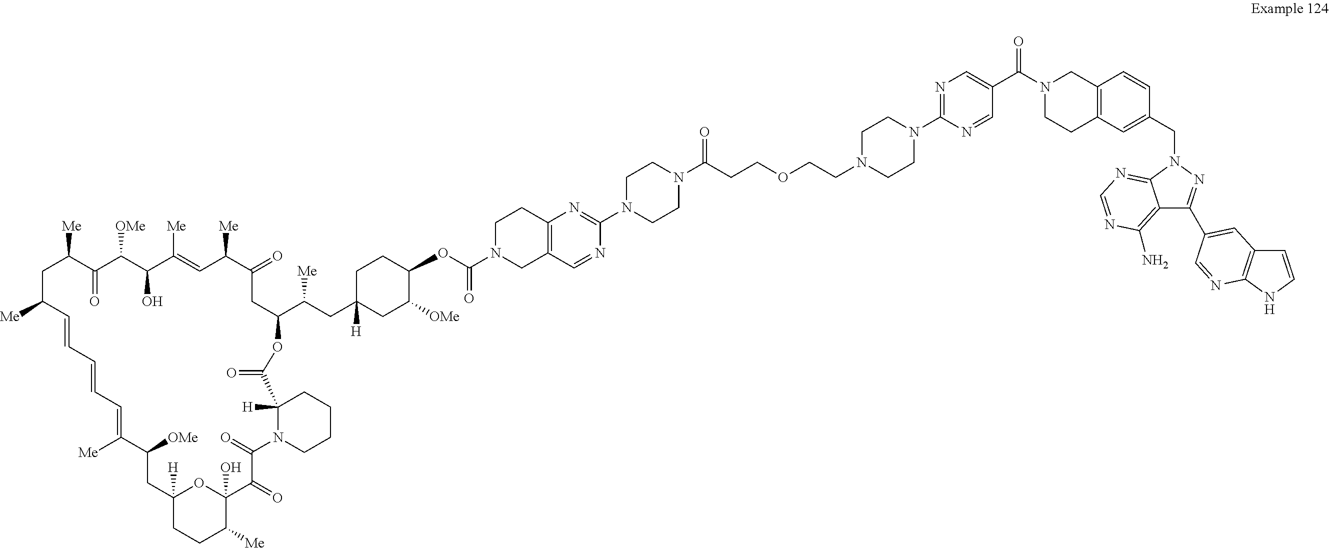 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ncc(C(=O)N8CCc9cc(Cn%10nc(-c%11cnc%12[nH]ccc%12c%11)c%11c(N)ncnc%11%10)ccc9C8)cn7)CC6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

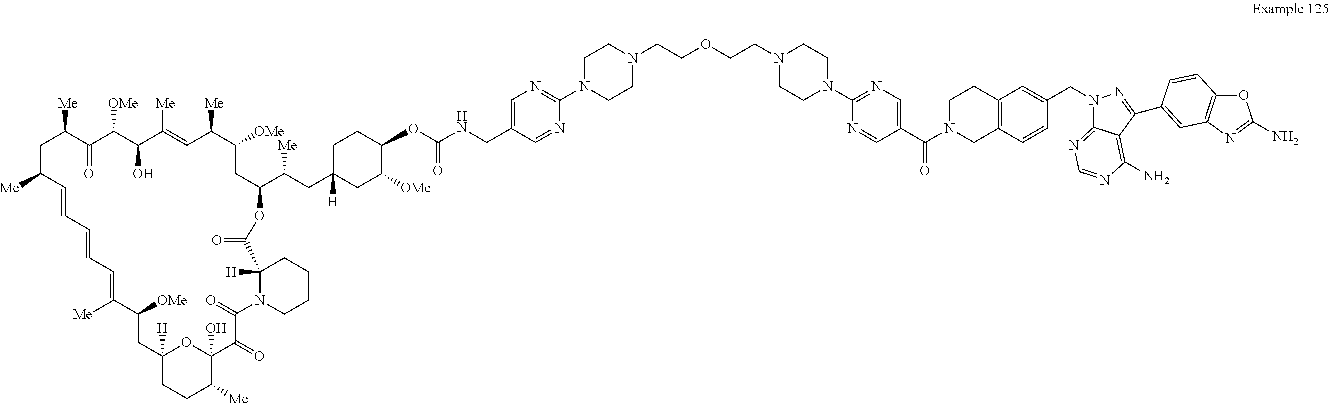 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

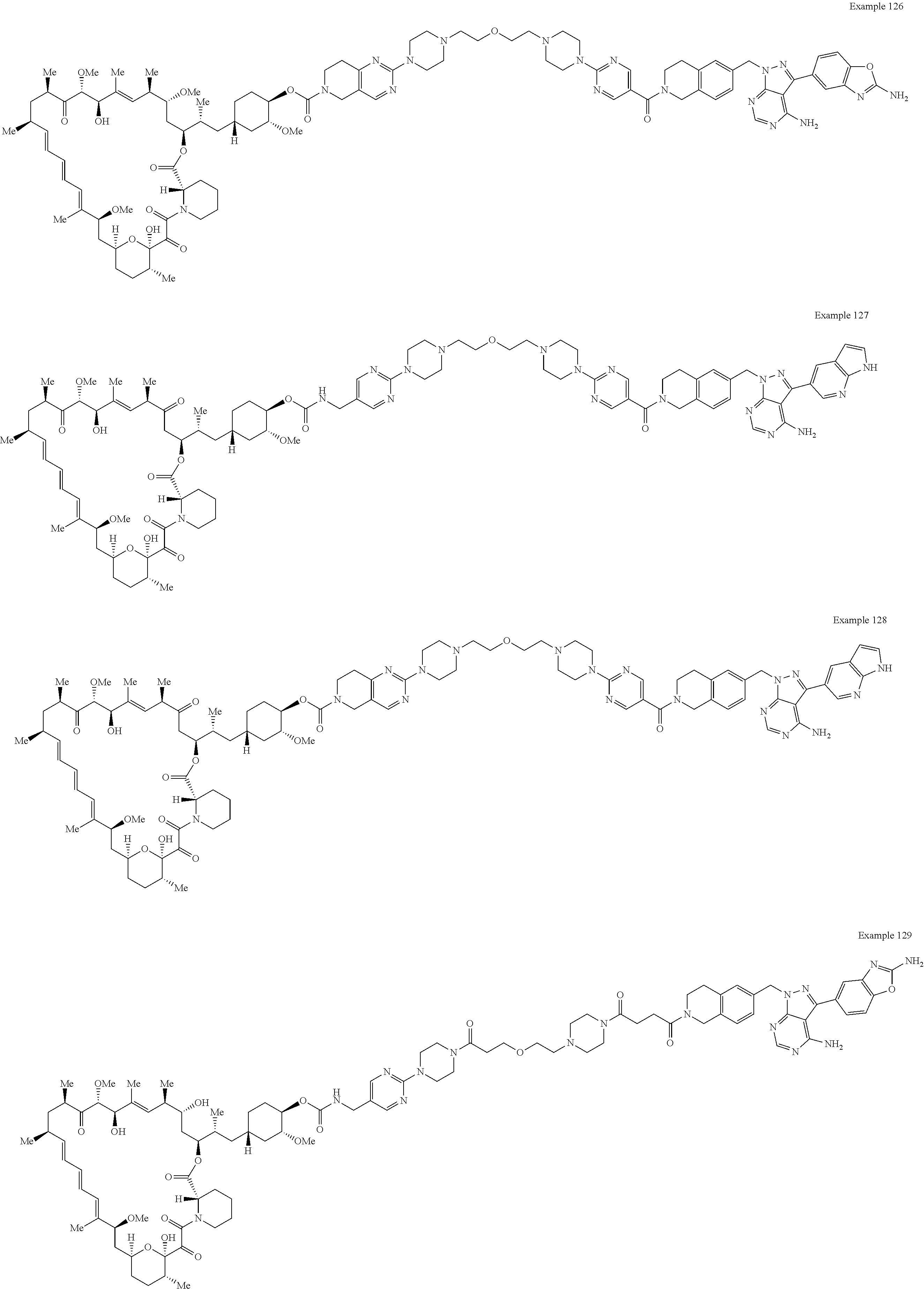

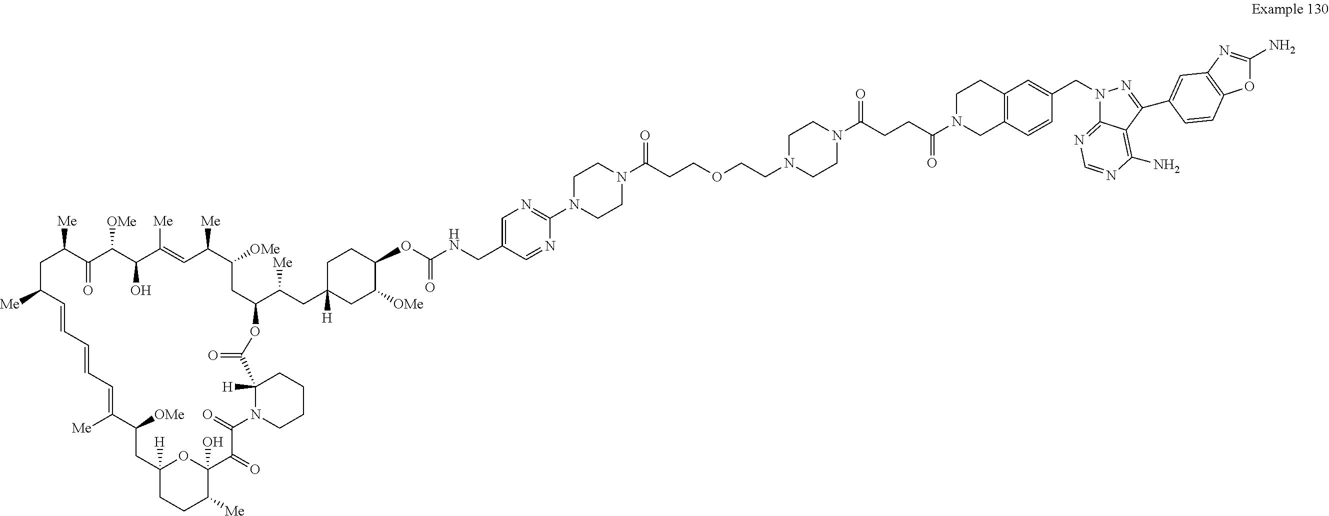 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)N6CCc7cc(Cn8nc(-c9ccc%10oc(N)nc%10c9)c9c(N)ncnc98)ccc7C6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

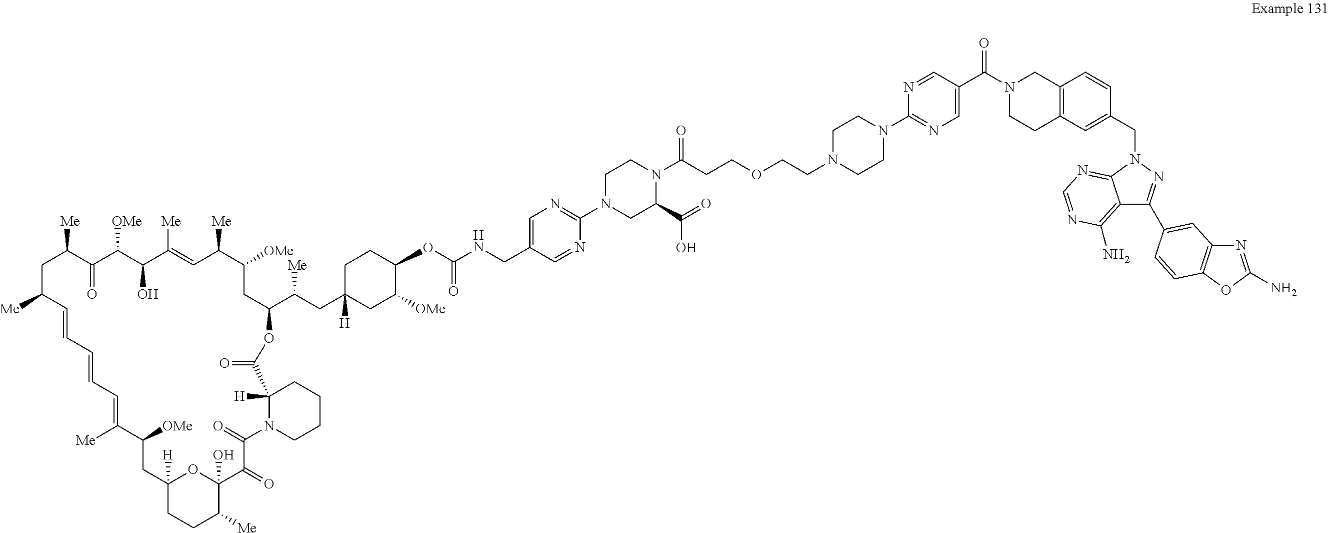 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)[C@@H](C(=O)O)C4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

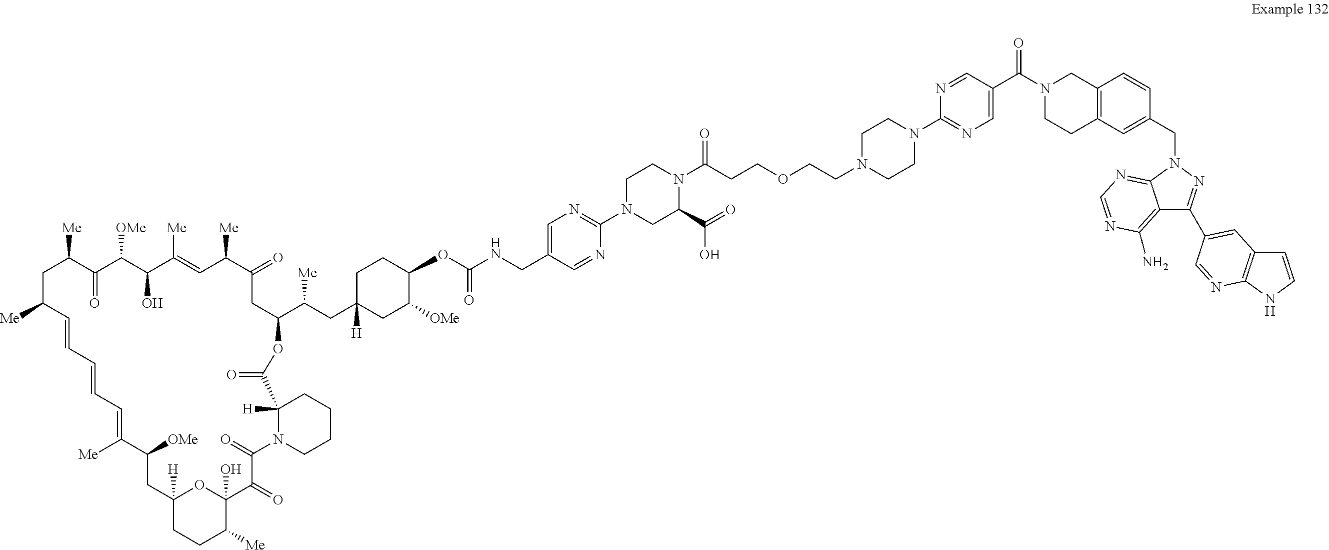 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)[C@@H](C(=O)O)C4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

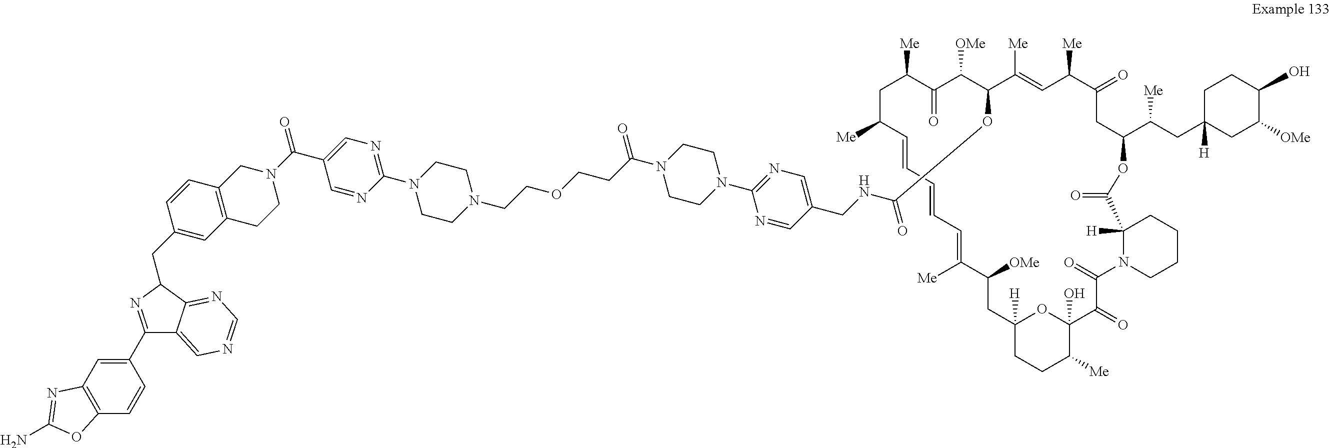 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](OC(=O)NCc2cnc(N3CCN(C(=O)CCOCCN4CCN(c5ncc(C(=O)N6CCc7cc(CC8N=C(c9ccc%10oc(N)nc%10c9)c9cncnc98)ccc7C6)cn5)CC4)CC3)nc2)[C@@H](OC)C(=O)[C@H](C)C[C@H](C)/C=C/C=C/C=C/1C]

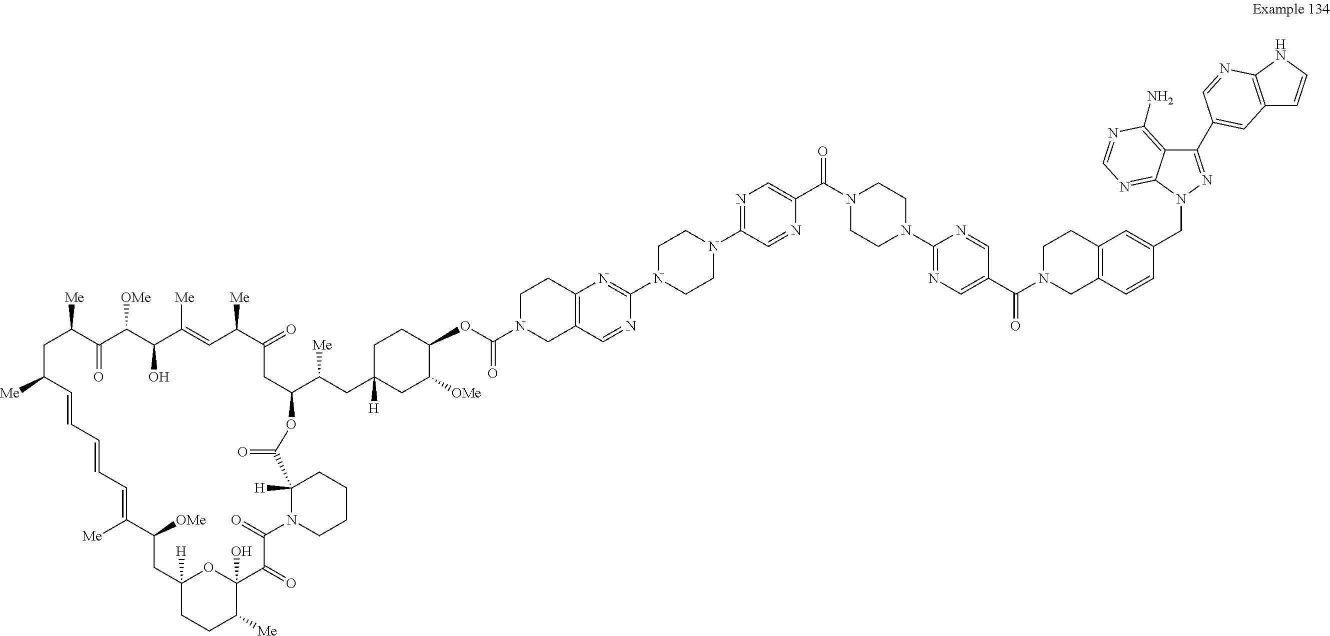 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6cnc(C(=O)N7CCN(c8ncc(C(=O)N9CCc%10cc(Cn%11nc(-c%12cnc%13[nH]ccc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

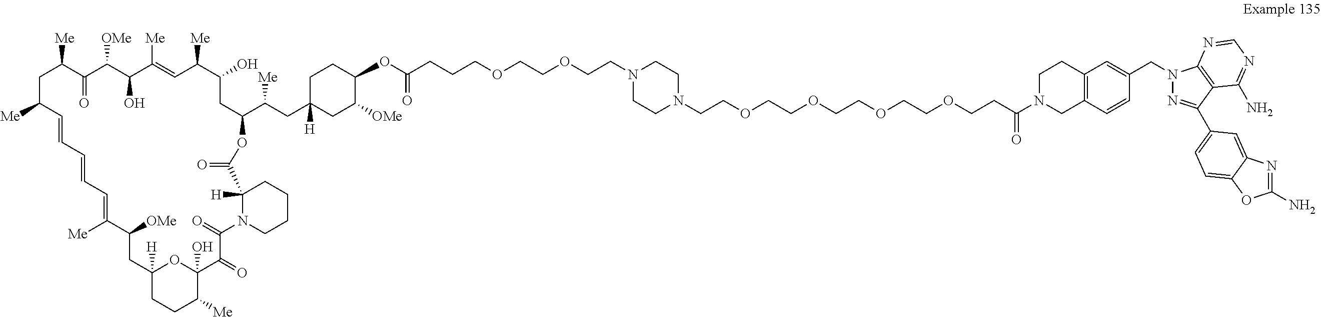 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)CCCOCCOCCN3CCN(CCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

In certain embodiments, the present disclosure provides for a compound selected from below or a pharmaceutically acceptable salt or tautomer thereof,

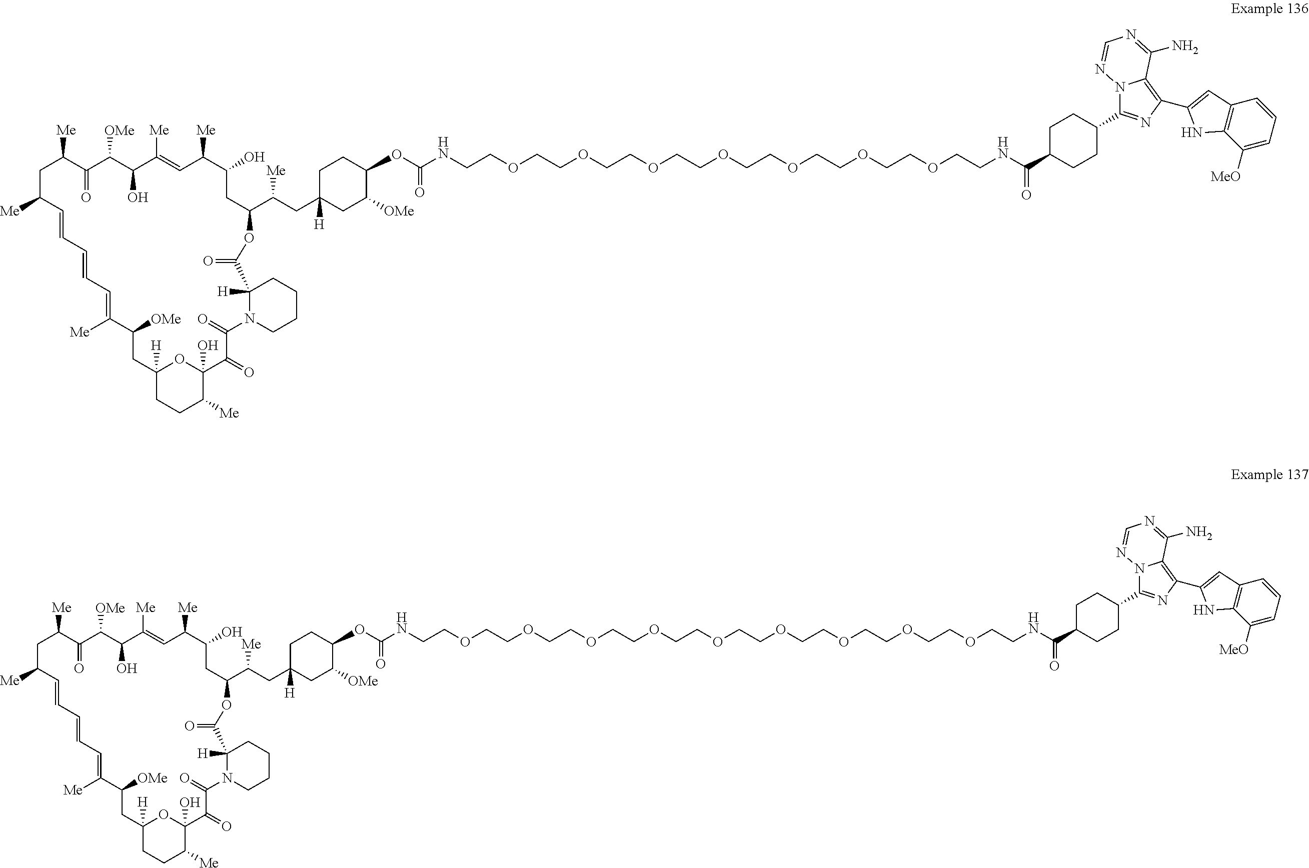

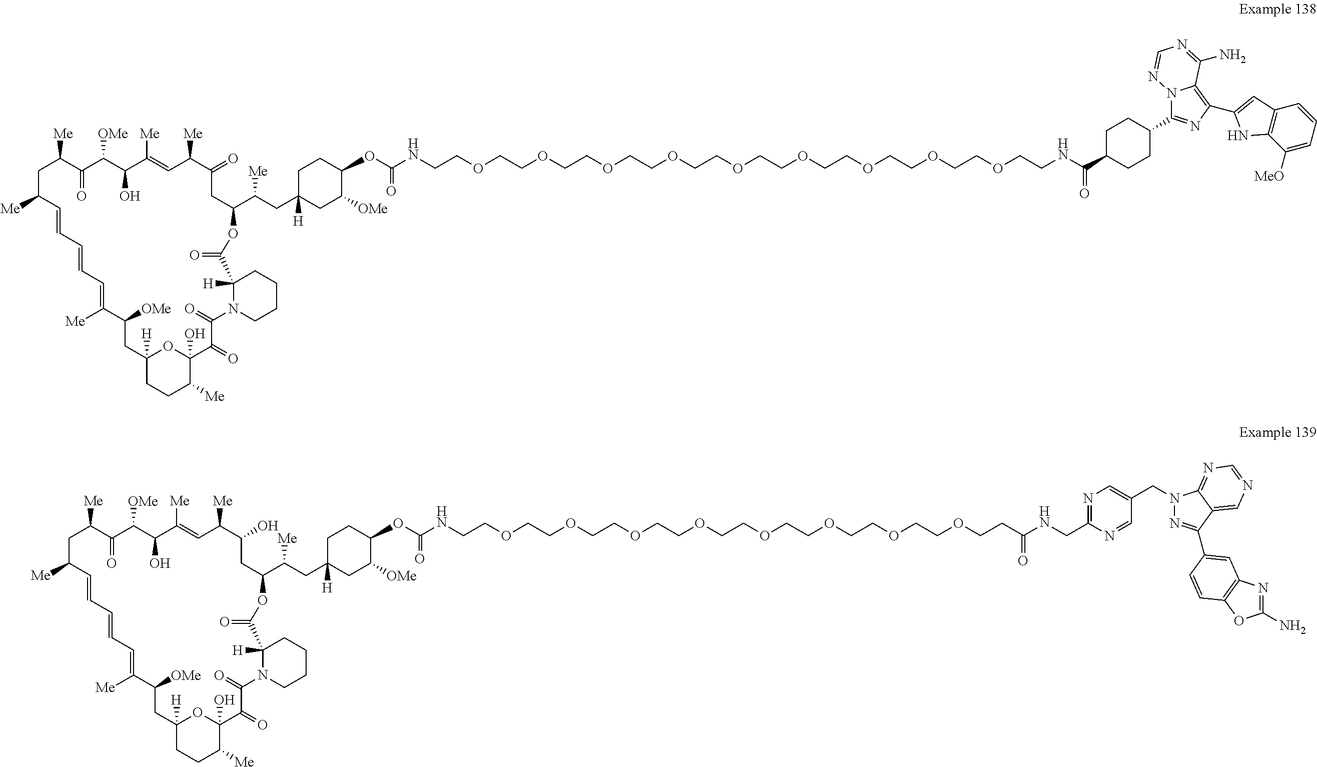

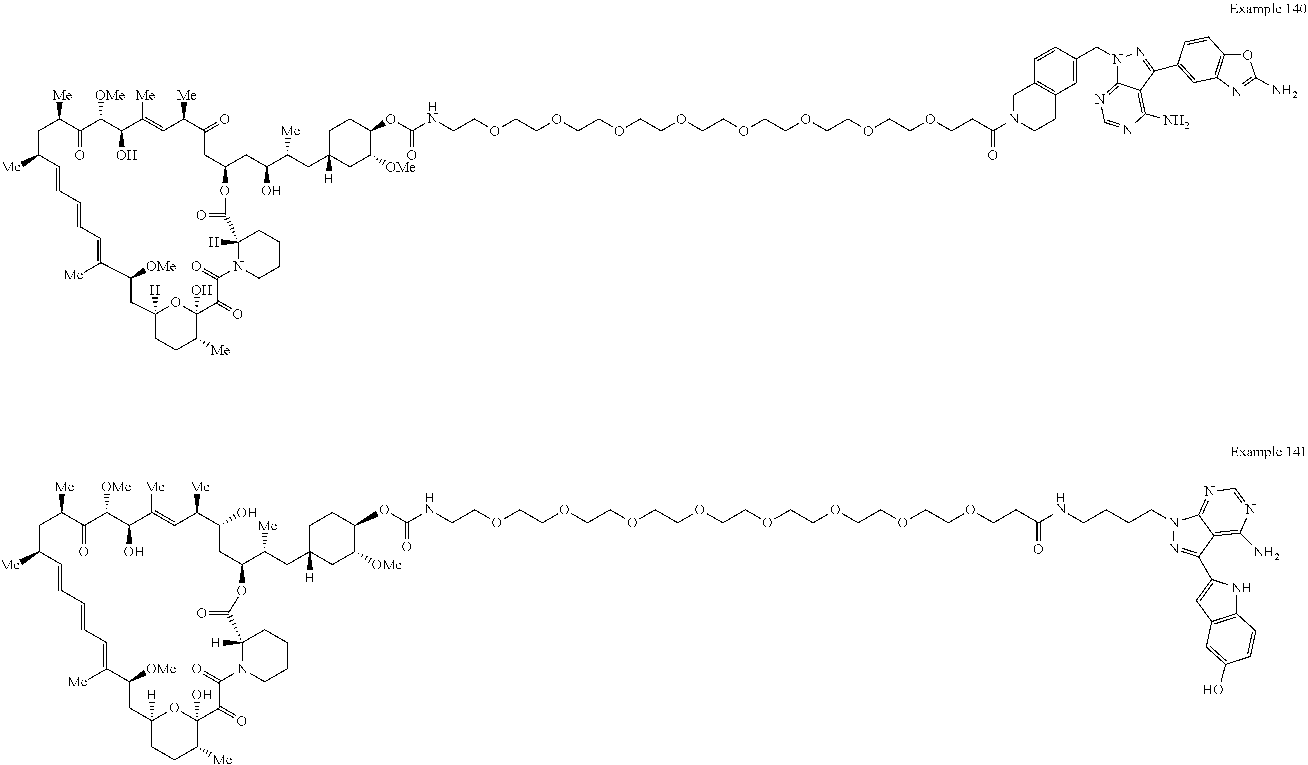

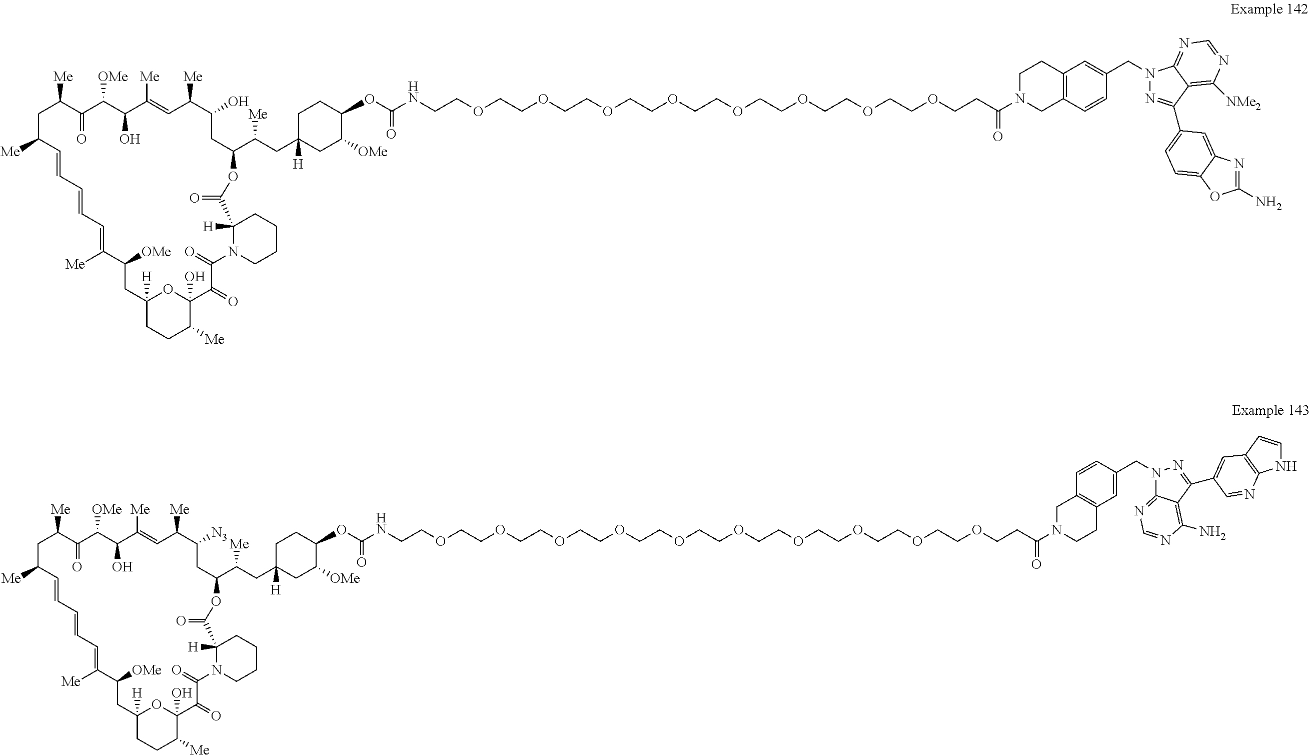

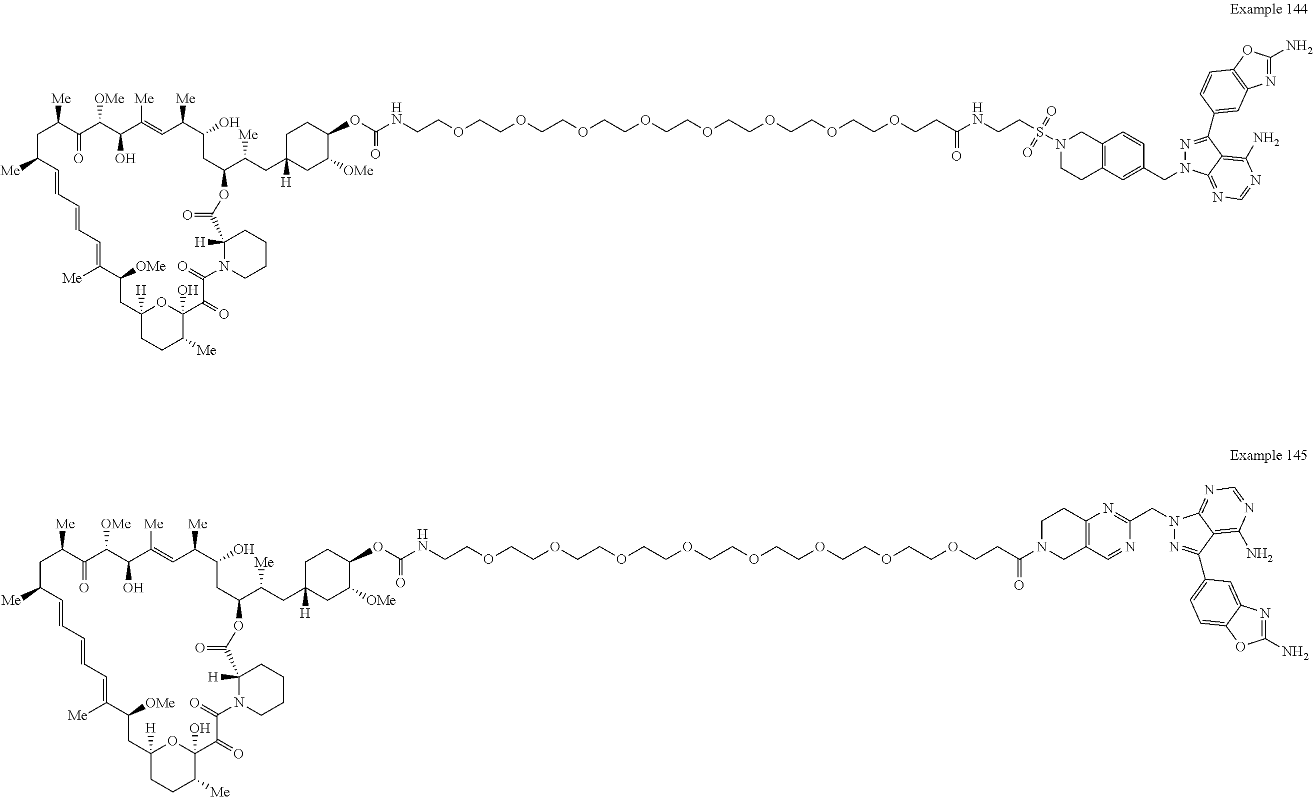

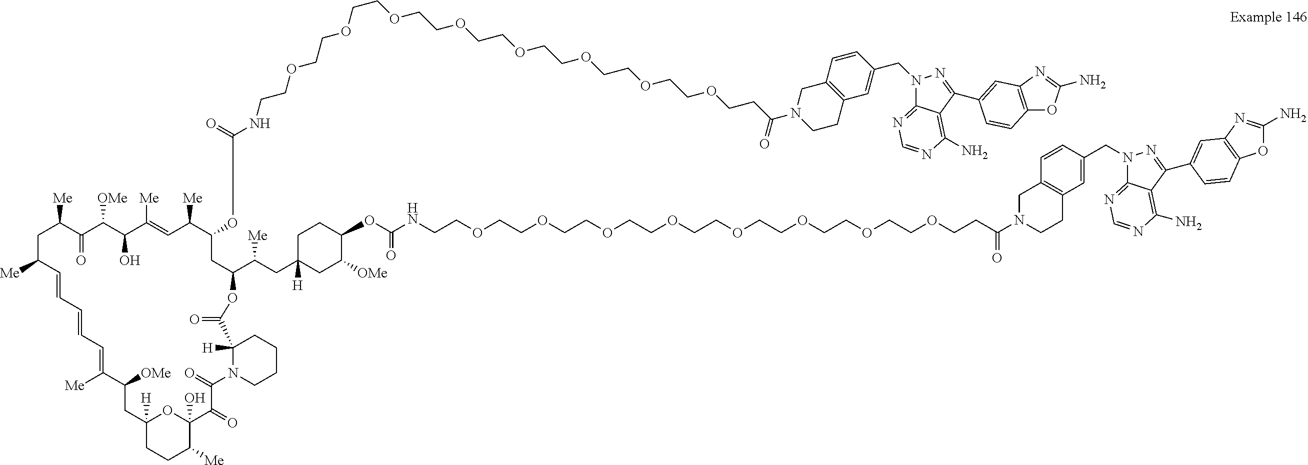 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC(=O)NCCOCCOCCOCCOCCOCCOCCOCCOCCC(=O)N2CCc3cc(Cn4nc(-c5ccc6oc(N)nc6c5)c5c(N)ncnc54)ccc3C2)[C@H](C)/C=C(\C)[C@@H](O)[C@@H](OC)C(=O)[C@H](C)C[C@H](C)/C=C/C=C/C=C/1C]

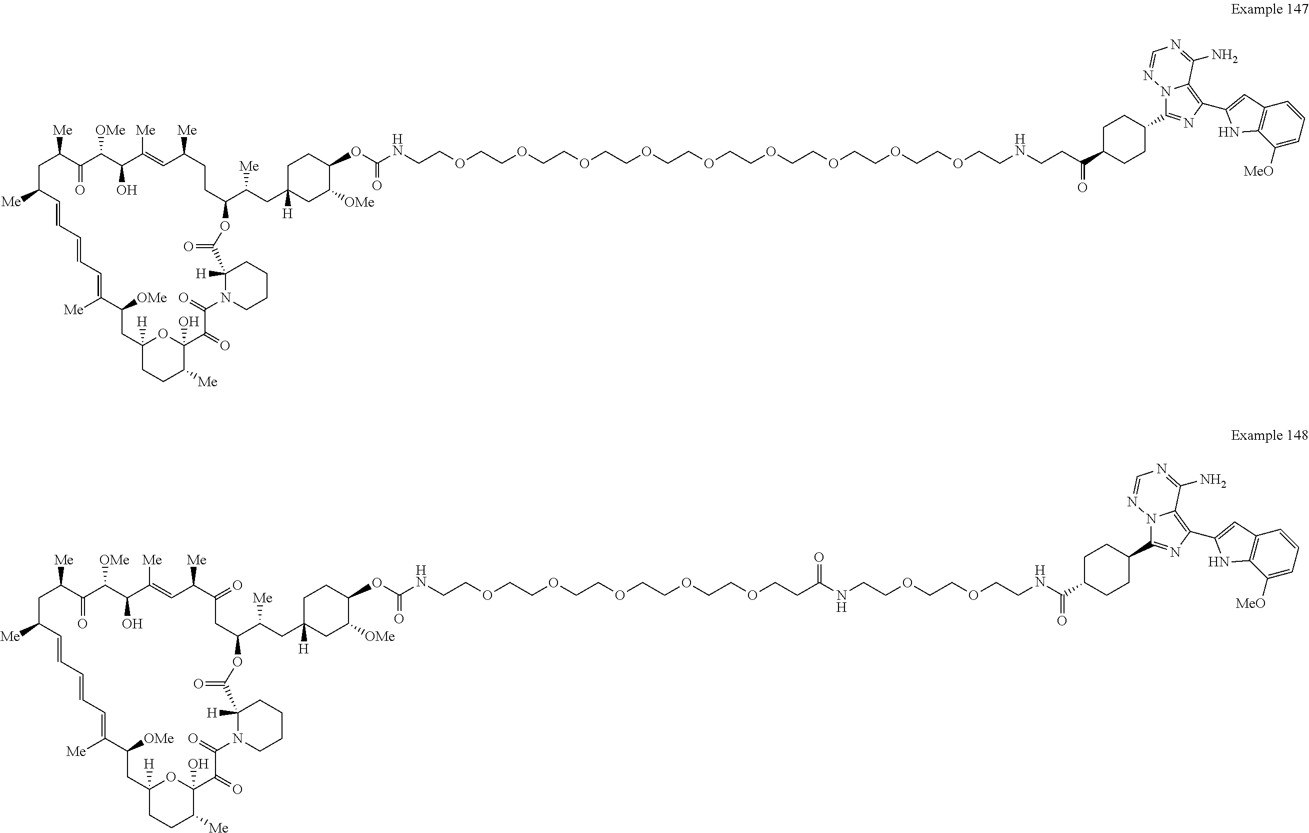

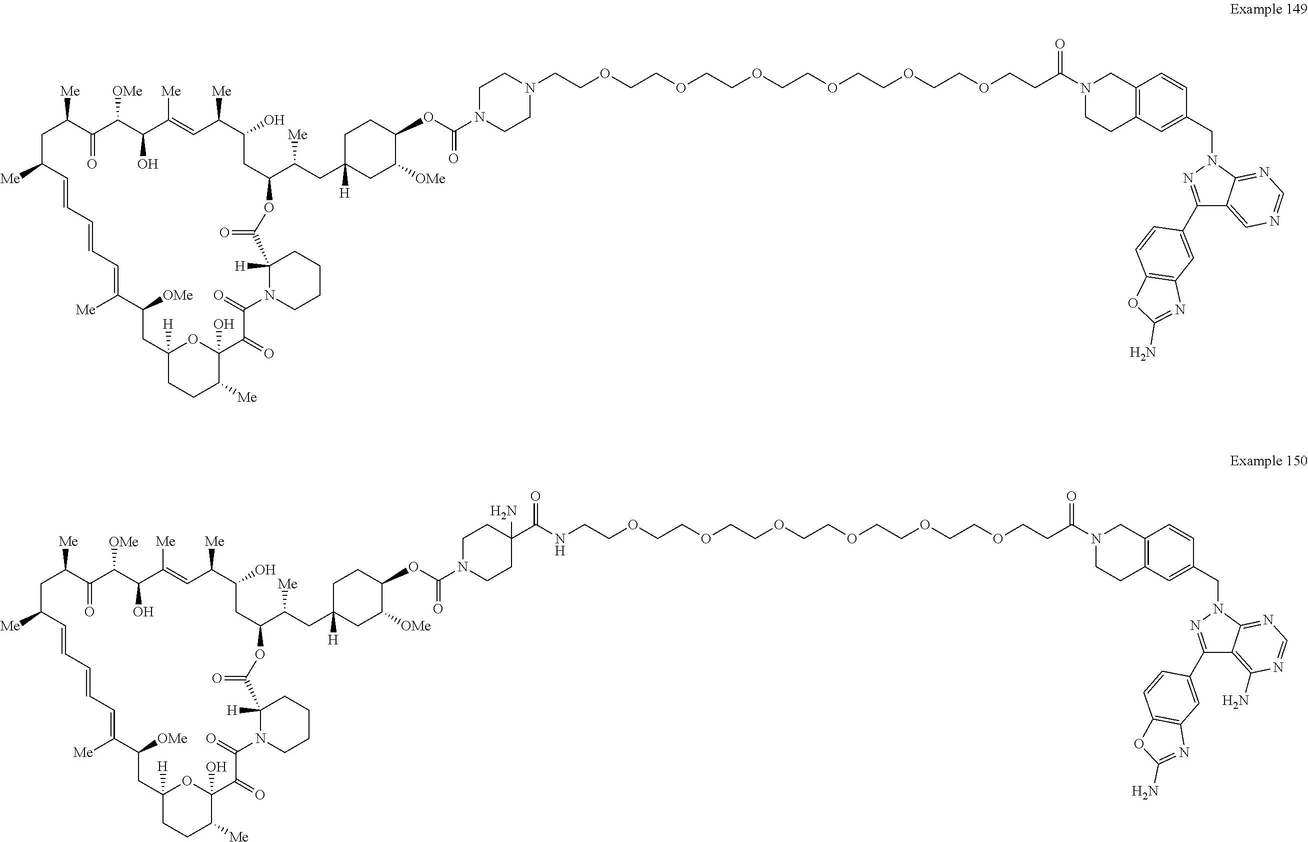

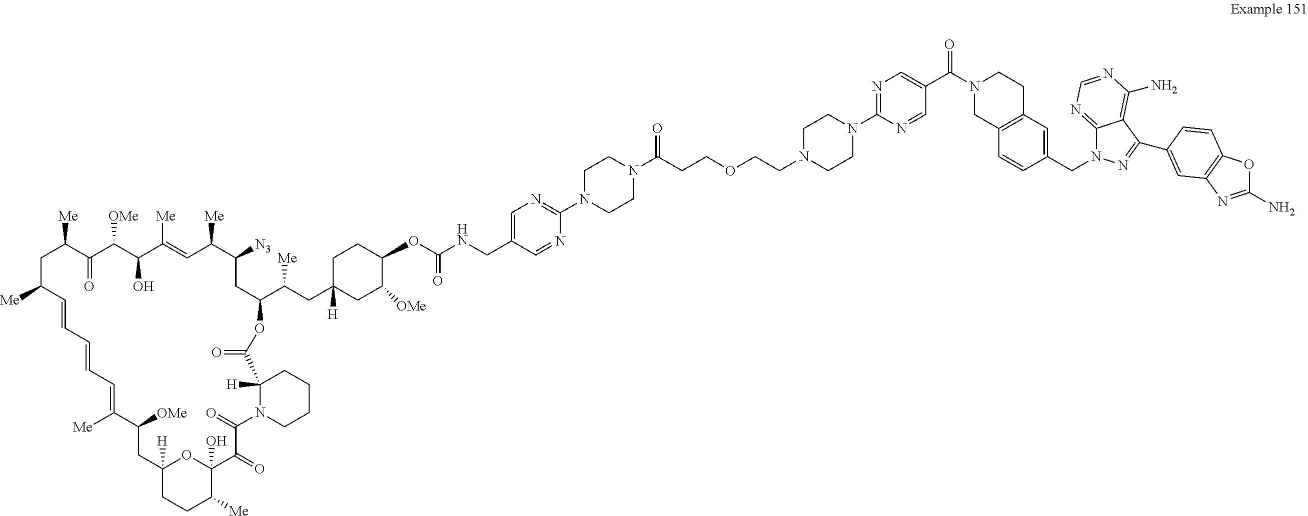 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C]

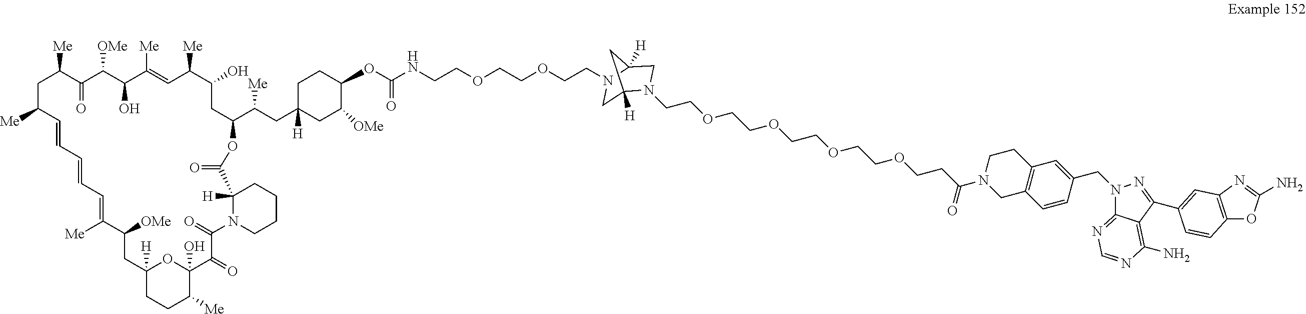 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCN3C[C@@H]4C[C@H]3CN4CCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

The compounds of the disclosure may include pharmaceutically acceptable salts of the compounds disclosed herein. Representative “pharmaceutically acceptable salts” may include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, 1,1-methene-bis-2-hydroxy-3-naphthoate, einbonate, pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

“Pharmaceutically acceptable salt” may also include both acid and base addition salts. “Pharmaceutically acceptable acid addition salt” may refer to those salts which retain the biological effectiveness and properties of the free bases, which are not biologically or otherwise undesirable, and which may be formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, l-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” may refer to those salts that retain the biological effectiveness and properties of the free acids, which are not biologically or otherwise undesirable. These salts may be prepared from addition of an inorganic base or an organic base to the free acid. Salts derived from inorganic bases may include, but are not limited to, the sodium, potassium, lithium, ammonium, calcium, magnesium, iron, zinc, copper, manganese, aluminum salts and the like. For example, inorganic salts may include, but are not limited to, ammonium, sodium, potassium, calcium, and magnesium salts. Salts derived from organic bases may include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like.

Unless otherwise stated, structures depicted herein may also include compounds which differ only in the presence of one or more isotopically enriched atoms. For example, compounds having the present structure except for the replacement of a hydrogen atom by deuterium or tritium, or the replacement of a carbon atom by ¹³C or ¹⁴C, or the replacement of a nitrogen atom by ¹⁵N, or the replacement of an oxygen atom with ¹⁷O or ¹⁸O are within the scope of the disclosure. Such isotopically labeled compounds are useful as research or diagnostic tools.

In some embodiments, one or more deuterium atoms may be introduced into the PEG moiety of any compound of the present invention. Mechanisms for such modifications are known in the art starting from commercially available starting materials, such as isotopically enriched hydroxylamine building blocks. In some embodiments, a tritium or a deuterium may be introduced at the C32 position of compounds of the present invention using, for example, a commercially available isotopically pure reducing agent and methods known to those in the art. In some embodiments, ¹⁴C may be introduced into the C40 carbamate moiety of compounds of the present invention using commercially available materials and methods known to those of skill in the art. In some embodiments, an isotope such as deuterium or tritium may be introduced into the R^{40a} substituent of a compound of Formula Ia, Ic, I or II, using commercially available starting materials and methods known to those of skill in the art.

### Methods of Synthesizing Disclosed Compounds

The compounds of the present disclosure may be made by a variety of methods, including standard chemistry. Suitable synthetic routes are depicted in the schemes given below.

The compounds of any of the formulae described herein may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of Formula I, Ia, Ib, II, or IIb, or a pharmaceutically acceptable salt or tautomer of any of the foregoing.

The compounds of any of the formulae described herein may be prepared by methods which avoid the use of metal-mediated cycloaddition reactions which require the use of azide-containing compounds. Azide containing compounds present potential safety hazards associated with their preparation and storage (e.g., explosion due to high energy decomposition). Also, the reaction schemes herein can avoid the use of copper or ruthenium metals in the penultimate or ultimate synthetic steps, which can be advantageous. Avoiding the use of copper or ruthenium metals in the penultimate or ultimate synthetic steps reduces the potential for contamination of the final compounds with undesirable metal impurities.

As rapamycin can be an expensive starting material, good yields on reactions are advantageous. The reaction schemes herein provide better yields than other reaction schemes. In the reaction schemes herein, there is no need to alkylate at the C40-hydroxyl of rapamycin, which is advantageous for providing as much as a 5-fold improved overall yield in preparing bivalent compounds from rapamycin compared to other reaction schemes.

There is an additional synthetic improvement associated with better yields. Avoiding the need to alkylate at the C40-hydroxyl gives as much as a 5-fold improved overall yield in preparing bivalent compounds from rapamycin.

Those skilled in the art will recognize if a stereocenter exists in any of the compounds of the present disclosure. Accordingly, the present disclosure may include both possible stereoisomers (unless specified in the synthesis) and may include not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be effected by any suitable method known in the art. See, for example, “Stereochemistry of Organic Compounds” by E. L. Eliel, S. H. Wilen, and L. N. Mander (Wiley-Interscience, 1994).

### Preparation of Compounds

The compounds described herein may be made from commercially available starting materials or synthesized using known organic, inorganic, and/or enzymatic processes.

The compounds of the present disclosure can be prepared in a number of ways well known to those skilled in the art of organic synthesis. By way of example, compounds of the disclosure can be synthesized using the methods described below, together with synthetic methods known in the art of synthetic organic chemistry, or variations thereon as appreciated by those skilled in the art. These methods may include but are not limited to those methods described below.

The term “tautomers” may refer to a set of compounds that have the same number and type of atoms, but differ in bond connectivity and are in equilibrium with one another. A “tautomer” is a single member of this set of compounds. Typically a single tautomer is drawn but it may be understood that this single structure may represent all possible tautomers that might exist. Examples may include enol-ketone tautomerism. When a ketone is drawn it may be understood that both the enol and ketone forms are part of the disclosure.

In addition to tautomers that may exist at all amide, carbonyl, and oxime groups within compounds of Formula I, Ia, Ib, Ic, II, or IIb, compounds in this family readily interconvert via a ring-opened species between two major isomeric forms, known as the pyran and oxepane isomers (shown below). This interconversion can be promoted by magnesium ions, mildly acidic conditions, or alkylamine salts, as described in the following references: i) Hughes, P. F.; Musser, J.; Conklin, M.; Russo, R. 1992. Tetrahedron Lett. 33(33): 4739-32. ii) Zhu, T. 2007. U.S. Pat. No. 7,241,771; Wyeth. iii) Hughes, P. F. 1994. U.S. Pat. No. 5,344,833; American Home Products Corp. The scheme below shows an interconversion between the pyran and oxepane isomers in compounds of Formula I, Ia, Ib, Ic, II, or IIb.

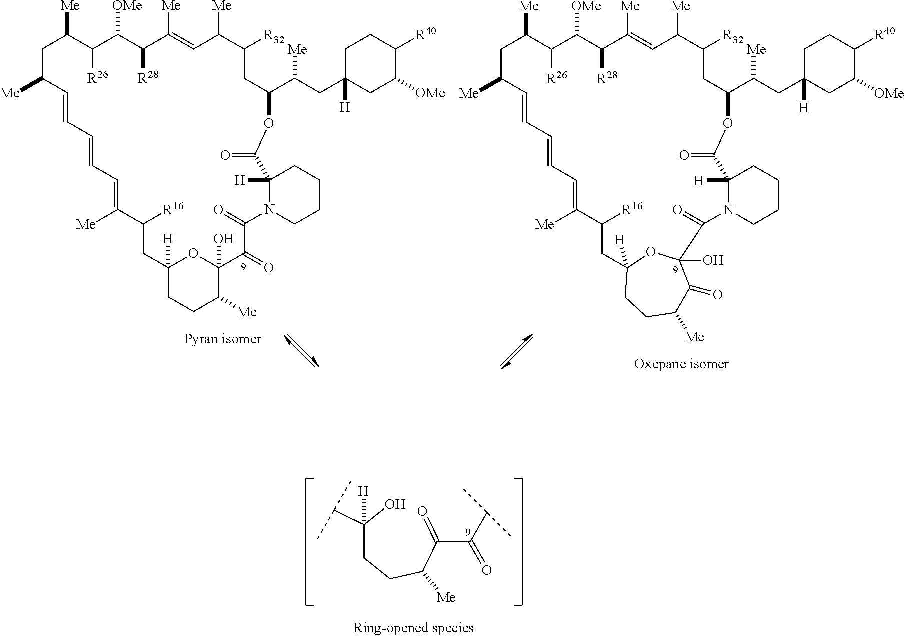

As this interconversion occurs under mild condition, and the thermodynamic equilibrium position may vary between different members of compounds of Formula I, Ia, Ib, Ic, II, or IIb, both isomers are contemplated for the compounds of Formula I, Ia, Ib, Ic, II, or IIb. For the sake of brevity, the pyran isomer form of all intermediates and compounds of Formula I, Ia, Ib, Ic, II, or IIb is shown.

### General Assembly Approaches for Bifunctional Rapalogs

With reference to the schemes below, rapamycin is Formula RAP,

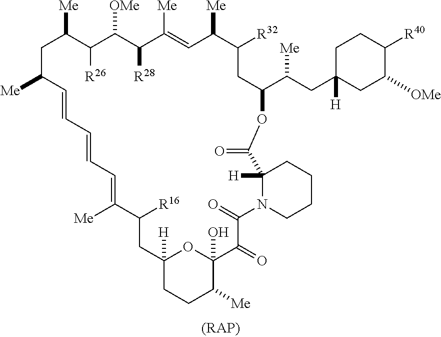

where R¹⁶ is —OCH₃; R²⁶ is ═O; R²⁸ is —OH; R³² is ═O; and R⁴⁰ is —OH. A “rapalog” refers to an analog or derivative of rapamycin. For example, with reference to the schemes below, a rapalog can be rapamycin that is substituted at any position, such as R¹⁶, R²⁶, R²⁸, R³², or R⁴⁰. An active site inhibitor (AS inhibitor) is an active site mTOR inhibitor. In certain embodiments, AS inhibitor is depicted by B, in Formula I, Ia, Ib, Ic, II, or IIb.

### Series 1 Bifunctional Rapalogs

A general structure of Series 1 bifunctional rapalogs is shown in Scheme 1 below. For these types of bifunctional rapalogs, the linker may include variations where q=0 to 30, such as q=1 to 7, and r=1 to 6. The linker amine can include substitutions, such as R═H and C₁-C₆ alkyl groups. The carbamate moiety, where Z¹═O or S, can be attached to the rapalog at R⁴⁰ or R²⁸ (Formula I, Ia, Tb, Ic, II, or IIb), including variations found in Table 1 in the Examples Section. An mTOR active site inhibitor can attach to the linker via a primary or secondary amine, and may include variations found in Table 2 in the Examples Section.

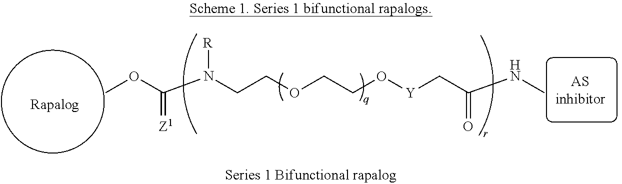

### Series 2 Bifunctional Rapalogs

A general structure of Series 2 bifunctional rapalogs is shown in Scheme 2 below. For these types of bifunctional rapalogs, the linker may include variations where q=0 to 30, such as q=1 to 7. The linker amine can include substitutions, such as R═H and C₁-C₆ alkyl groups. The pre-linker amine can include substitutions, such as R²═H, C₁-C₆ alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

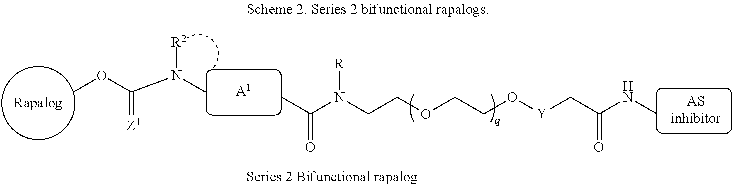

### Series 3 Bifunctional Rapalogs

A general structure of Series 3 bifunctional rapalogs is shown in Scheme 3 below. For these types of bifunctional rapalogs, the linker may include variations where q=0 to 30, such as q=1 to 7. The linker amine can include substitutions, such as R═H and C₁-C₆ alkyl groups. The post-linker amine can include substitutions, such as R²═H, C₁-C₆ alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

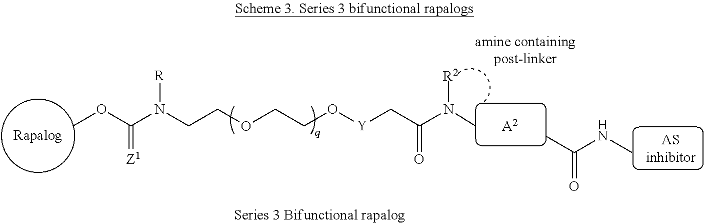

### Series 4 Bifunctional Rapalogs

A general structure of Series 4 bifunctional rapalogs is shown in Scheme 4 below. For these types of bifunctional rapalogs, the linker may include variations where q=0 to 30, such as q=1 to 7. The linker amine can include substitutions, such as R═H and C₁-C₆ alkyl groups. The pre- and post-linker amines can each include substitutions, such as R²═H, C₁-C₆ alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

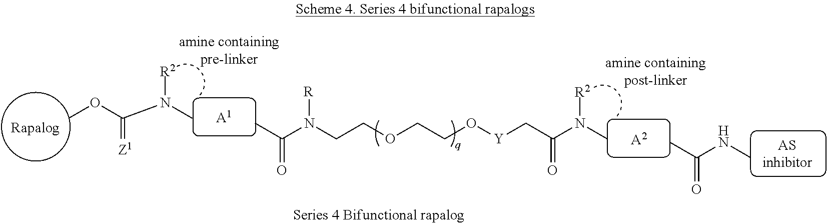

### Series 5 Bifunctional Rapalogs

A general structure of Series 5 bifunctional rapalogs is shown in Scheme 5 below. For these types of bifunctional rapalogs, the pre-linker amine can include substitutions, such as R²═H, C₁-C₆ alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

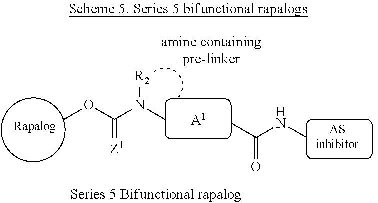

### Series 6 Bifunctional Rapalogs

A general structure of Series 6 bifunctional rapalogs is shown in Scheme 6 below. For these types of bifunctional rapalogs, the linker may include variations where q=0 to 30, such as q=1 to 7. The linker amines can include substitutions, such as R═H and C₁-C₆ alkyl groups. The post-linker amine can include substitutions, such as R²═H, C₁-C₆ alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

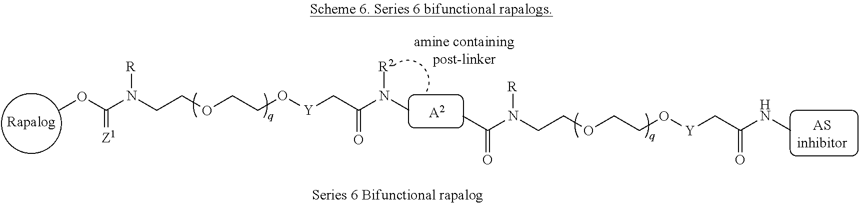

### Series 7 Bifunctional Rapalogs

A general structure of Series 7 bifunctional rapalogs is shown in Scheme 7 below. For these types of bifunctional rapalogs, the linker may include variations where q=0 to 30, such as q=1 to 7. The linker amine can include substitutions, such as R═H and C₁-C₆ alkyl groups. The pre- and post-linker amines can each include substitutions, such as R²═H, C₁-C₆ alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

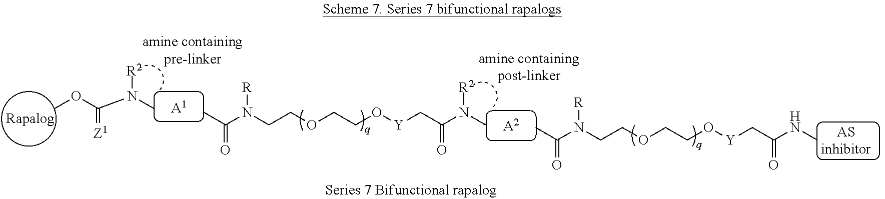

### Series 8 Bifunctional Rapalogs

A general structure of Series 8 bifunctional rapalogs is shown in Scheme 8 below. For these types of bifunctional rapalogs, the linker may include variations where q=0 to 30, such as q=1 to 7. The linker amine can include substitutions, such as R═H and C₁-C₆ alkyl groups. The post-linker amine can include substitutions, such as R²═H, C₁-C₆ alkyl groups, and cycloalkyl including 4 to 8-membered rings. The carbamate moiety, where Z¹═O or S, can be attached to the rapalog at R⁴⁰ or R²⁸ (Formula I, Ia, Ib, Ic, II, or IIb), including variations found in Table 1 in the Examples Section. An mTOR active site inhibitor can attach to the linker via a primary or secondary amine, and may include variations found in Table 2 in the Examples Section.

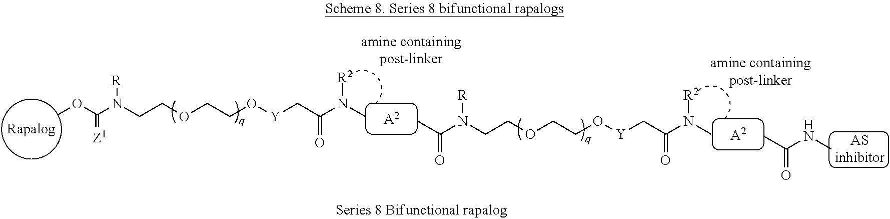

### Pharmaceutical Compositions

Another aspect provides a pharmaceutical composition including a pharmaceutically acceptable excipient and a compound of the present invention, or pharmaceutically acceptable salt or tautomer thereof.

In embodiments of the pharmaceutical compositions, a compound of the present invention, or a pharmaceutically acceptable salt or tautomer thereof, may be included in a therapeutically effective amount.

Administration of the disclosed compounds or compositions can be accomplished via any mode of administration for therapeutic agents. These modes may include systemic or local administration such as oral, nasal, parenteral, transdermal, subcutaneous, vaginal, buccal, rectal, topical, intrathecal, or intracranial administration modes.

In certain embodiments, administering can include oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration can be by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Set Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989). The compositions of the present disclosure can also be delivered as nanoparticles.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, intrathecal, subcutaneous or intramuscular form, and all using forms well known to those skilled in the pharmaceutical arts.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the disclosure relates to a pharmaceutical composition comprising a compound, or a pharmaceutically acceptable salt or tautomer thereof, of the present disclosure and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The compounds described herein can be used in combination with one another, with other active agents known to be useful in treating cancer, autoimmune disease, inflammatory disease, metabolic disease, neurodegenerative disease, fungal infection, or transplant rejection, or with adjunctive agents that may not be effective alone, but may contribute to the efficacy of the active agent. The compounds described herein can be used in combination with other active agents known to be longevity agents or anti-aging agents.

In embodiments of the pharmaceutical compositions, the pharmaceutical composition may include a second agent (e.g., therapeutic agent). In embodiments of the pharmaceutical compositions, the pharmaceutical composition may include a second agent (e.g., therapeutic agent) in a therapeutically effective amount. In certain embodiments, the second agent is an anti-cancer agent. In certain embodiments, the second agent is an immunotherapeutic agent. In certain embodiments, the second agent is an immune-oncological agent. In certain embodiments, the second agent is an anti-autoimmune disease agent. In embodiments, the second agent is an anti-inflammatory disease agent. In certain embodiments, the second agent is an anti-neurodegenerative disease agent. In certain embodiments, the second agent is an anti-metabolic disease agent. In certain embodiments, the second agent is an anti-cardiovascular disease agent. In certain embodiments, the second agent is an anti-aging agent. In certain embodiments, the second agent is a longevity agent. In certain embodiments, the second agent is an agent for treating or preventing transplant rejection. In certain embodiments, the second agent is an agent for treating or preventing fungal infection. In certain embodiments, the second agent is immune system repressor. In certain embodiments, the second agent is an mTOR modulator. In certain embodiments, the second agent is an mTOR inhibitor. In certain embodiments, the second agent is an active site mTOR inhibitor. In certain embodiments, the second agent is a rapamycin. In certain embodiments, the second agent is a rapamycin analog. In certain embodiments, the second agent is an mTOR pathway inhibitor. In certain embodiments, the second agent is CDK4/6 inhibitor; anti-PD1/PD-L1, PI3K inhibitor; or Ras inhibitor.

“Anti-cancer agent” or “anti-cancer drug” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anticancer agent is an agent approved by the FDA or similar regulatory agency of a country other than the USA, for treating cancer. Examples of anti-cancer agents include, but are not limited to, rapamycin, rapamycin analog, bevacizumab, PP242, ΓN 128, MLN0128, anti-androgens (e.g., Casodex, Flutamide, MDV3100, or ARN-509), MEK (e.g. MEK1, MEK2, or MEK1 and MEK2) inhibitors (e.g. XL518, CI-1040, PD035901, selumetinib/AZD6244, GSK1 120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP 16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ^{U1}ln, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pyrrolo benzodiazepines (e.g. tomaymycin), carboplatin, CC-1065 and CC-1065 analogs including amino-CBIs, nitrogen mustards (such as chlorambucil and melphalan), dolastatin and dolastatin analogs (including auristatins: eg. monomethyl auristatin E), anthracycline antibiotics (such as doxorubicin, daunorubicin, etc.), duocarmycins and duocarmycin analogs, enediynes (such as neocarzinostatin and calicheamicins), leptomycin derivaties, maytansinoids and maytansinoid analogs (e.g. mertansine), methotrexate, mitomycin C, taxoids, vinca alkaloids (such as vinblastine and vincristine), epothilones (e.g. epothilone B), camptothecin and its clinical analogs topotecan and irinotecan, FNK128, PP242, PP121, MLN0128, AZD8055, AZD2014, VP-BEZ235, BGT226, SFl 126, Torin 1, Torin 2, WYE 687, WYE 687 salt (e.g., hydrochloride), PF04691502, PI-103, CC-223, OSI-027, XL388, KU-0063794, GDC-0349, PKI-587, rapamycin, deforolimus (AP23573, MK-8669, ridaforolimus), temsirolimus (CCI-779), ABT478, everolimus (RAD001) or the like.

### mTOR and Methods of Treatment

The term “mTOR” refers to the protein “mechanistic target of rapamycin (serine/threonine kinase)” or “mammalian target of rapamycin.” The term “mTOR” may refer to the nucleotide sequence or protein sequence of human mTOR (e.g., Entrez 2475, Uniprot P42345, RefSeq NM_004958, or RefSeq NP_004949) (SEQ ID NO: 1). The term “mTOR” may include both the wild-type form of the nucleotide sequences or proteins as well as any mutants thereof. In some embodiments, “mTOR” is wild-type mTOR. In some embodiments, “mTOR” is one or more mutant forms. The term “mTOR” XYZ may refer to a nucleotide sequence or protein of a mutant mTOR wherein the Y numbered amino acid of mTOR that normally has an X amino acid in the wildtype, instead has a Z amino acid in the mutant. In embodiments, an mTOR is the human mTOR. In embodiments, the mTOR has the nucleotide sequence corresponding to reference number GL206725550 (SEQ ID NO:2). In embodiments, the mTOR has the nucleotide sequence corresponding to RefSeq NM_004958.3 (SEQ ID NO:2). In embodiments, the mTOR has the protein sequence corresponding to reference number GL4826730 (SEQ ID NO: 1). In embodiments, the mTOR has the protein sequence corresponding to RefSeq NP_004949.1 (SEQ ID NO: 1). In embodiments, the mTOR has the following amino acid sequence:

(SEQ ID NO: 1)
MLGTGPAAATTAATTSSNVSVLQQFASGLKSRNEETRAKAAKELQHYVTM
ELREMSQEESTRFYDQLNHHIFELVSSSDANERKGGILAIASLIGVEGGN
ATRIGRFANYLRNLLPSNDPWMEMASKAIGRLAMAGDTFTAEYVEFEVKR
ALEWLGADRNEGRRHAAVLVLRELAISVPTFFFQQVQPFFDNIFVAVWDP
KQAIREGAVAALRACLILTTQREPKEMQKPQWYRHTFEEAEKGFDETLAK
EKGMNRDDRIHGALLILNELVRISSMEGERLREEMEEITQQQLVHDKYCK
DLMGFGTKPRHITPFTSFQAVQPQQSNALVGLLGYSSHQGLMGFGTSPSP
AKSTLVESRCCRDLMEEKFDQVCQWVLKCRNSKNSLIQMTILNLLPRLAA
FRPSAFTDTQYLQDTMNHVLSCVKKEKERTAAFQALGLLSVAVRSEFKVY
LPRVLDIIRAALPPKDFAHKRQKAMQVDATVFTCISMLARAMGPGIQQDI
KELLEPMLAVGLSPALTAVLYDLSRQIPQLKKDIQDGLLKMLSLVLMHKP
LRHPGMPKGLAHQLASPGLTTLPEASDVGSITLALRTLGSFEFEGHSLTQ
FVRHCADHFLNSEHKEIRMEAARTCSRLLTPSIHLISGHAHVVSQTAVQV
VADVLSKLLWGITDPDPDIRYCVLASLDERFDAHLAQAENLQALFVALND
QVFEIRELAICTVGRLSSMNPAFVMPFLRKMLIQILTELEHSGIGRIKEQ
SARMLGHLVSNAPRLIRPYMEPILKALILKLKDPDPDPNPGVINNVLATI
GELAQVSGLEMRKWVDELFIIIMDMLQDSSLLAKRQVALWTLGQLVASTG
YVVEPYRKYPTLLEVLLNFLKTEQNQGTRREAIRVLGLLGALDPYKHKVN
IGMIDQSRDASAVSLSESKSSQDSSDYSTSEMLVNMGNLPLDEFYPAVSM
VALMRIFRDQSLSHHHTMVVQAITFIFKSLGLKCVQFLPQVMPTFLNVIR
VCDGAIREFLFQQLGMLVSFVKSHIRPYMDEIVTLMREFWVMNTSIQSTI
ILLIEQIVVALGGEFKLYLPQLIPHMLRVFMHDNSPGRIVSIKLLAAIQL
FGANLDDYLHLLLPPIVKLFDAPEAPLPSRKAALETVDRLTESLDFTDYA
SRIIHPIVRTLDQSPELRSTAMDTLSSLVFQLGKKYQIFIPMVNKVLVRH
RINHQRYDVLICRIVKGYTLADEEEDPLIYQHRMLRSGQGDALASGPVET
GPMKKLHVSTINLQKAWGAARRVSKDDWLEWLRRLSLELLKDSSSPSLRS
CWALAQAYNPMARDLFNAAFVSCWSELNEDQQDELIRSIELALTSQDIAE
VTQTLLNLAEFMEHSDKGPLPLRDDNGIVLLGERAAKCRAYAKALHYKEL
EFQKGPTPAILESLISINNKLQQPEAAAGVLEYAMKHFGELEIQATWYEK
LHEWEDALVAYDKKMDTNKDDPELMLGRMRCLEALGEWGQLHQQCCEKWT
LVNDETQAKMARMAAAAAWGLGQWDSMEEYTCMIPRDTHDGAFYRAVLAL
HQDLFSLAQQCIDKARDLLDAELTAMAGESYSRAYGAMVSCHMLSELEEV
IQYKLVPERREIIRQIWWERLQGCQRIVEDWQKILMVRSLVVSPHEDMRT
WLKYASLCGKSGRLALAHKTLVLLLGVDPSRQLDHPLPTVHPQVTYAYMK
NMWKSARKIDAFQHMQHFVQTMQQQAQHAIATEDQQHKQELHKLMARCFL
KLGEWQLNLQGINESTIPKVLQYYSAATEHDRSWYKAWHAWAVMNFEAVL
HYKHQNQARDEKKKLRHASGANITNATTAATTAATATTTASTEGSNSESE
AESTENSPTPSPLQKKVTEDLSKTLLMYTVPAVQGFFR SISLSRGNNLQ
DTLRVLTLWFDYGHWPDVNEALVEGVKAIQIDTWLQVIPQLIARIDTPRP
LVGRLIHQLLTDIGRYHPQALIYPLTVASKSTTTARHNAANKILKNMCEH
SNTLVQQAMMVSEELIRVAILWHEMWHEGLEEASRLYFGERNVKGMFEVL
EPLHAMMERGPQTLKETSFNQAYGRDLMEAQEWCRKYMKSGNVKDLTQA
WDLYYHVFRRISKQLPQLTSLELQYVSPKLLMCRDLELAVPGTYDPNQPI
IRIQSIAPSLQVITSKQRPRKLTLMGSNGHEFVFLLKGHEDLRQDERVMQ
LFGLVNTLLANDPTSLRKNLSIQRYAVIPLSTNSGLIGWVPHCDTLHALI
RDYREKKKILLNIEHRIMLRMAPDYDHLTLMQKVEVFEHAVNNTAGDDLA
KLLWLKSPSSEVWFDRRTNYTRSLAVMSMVGYILGLGDRHPSNLMLDRLS
GKILHIDFGDCFEVAMTREKFPEKIPFRLTRMLTNAMEVTGLDGNYRITC
HTVMEVLREHKDSVMAVLEAFVYDPLLNWRLMDTNTKGNKRSRTRTDSYS
AGQSVEILDGVELGEPAHKKTGTTVPESIHSFIGDGLVKPEALNKKAIQ
IINRVRDKLTGRDFSHDDTLDVPTQVELLIKQATSHENLCQCYIGWCPFW

In embodiments, the mTOR is a mutant mTOR. In embodiments, the mutant mTOR is associated with a disease that is not associated with wildtype mTOR. In embodiments, the mTOR may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to the sequence above.

The term “mTORC1” refers to the protein complex including mTOR and Raptor (regulatory-associated protein of mTOR). mTORC1 may also include MLST8 (mammalian lethal with SEC 13 protein 8), PRAS40, and/or DEPTOR. mTORC1 may function as a nutrient/energy/redox sensor and regulator of protein synthesis. The term “mTORC1 pathway” or “mTORC1 signal transduction pathway” may refer to a cellular pathway including mTORC1. An mTORC1 pathway includes the pathway components upstream and downstream from mTORC1. An mTORC1 pathway is a signaling pathway that is modulated by modulation of mTORC1 activity. In embodiments, an mTORC1 pathway is a signaling pathway that is modulated by modulation of mTORC1 activity but not by modulation of mTORC2 activity. In embodiments, an mTORC1 pathway is a signaling pathway that is modulated to a greater extent by modulation of mTORC1 activity than by modulation of mTORC2 activity.

The term “mTORC2” refers to the protein complex including mTOR and RICTOR (rapamycin-insensitive companion of mTOR). mTORC2 may also include GβL, mSIN1 (mammalian stress-activated protein kinase interacting protein 1), Protor 1/2, DEPTOR, TTI1, and/or TEL2. mTORC2 may regulate cellular metabolism and the cytoskeleton. The term “mTORC2 pathway” or “mTORC2 signal transduction pathway” may refer to a cellular pathway including mTORC2. An mTORC2 pathway includes the pathway components upstream and downstream from mTORC2. An mTORC2 pathway is a signaling pathway that is modulated by modulation of mTORC2 activity. In embodiments, an mTORC2 pathway is a signaling pathway that is modulated by modulation of mTORC2 activity but not by modulation of mTORC1 activity. In embodiments, an mTORC2 pathway is a signaling pathway that is modulated to a greater extent by modulation of mTORC2 activity than by modulation of mTORC1 activity.

The term “rapamycin” or “sirolimus” refers to a macrolide produced by the bacteria Streptomyces hygroscopicus. Rapamycin may prevent the activation of T cells and B cells. Rapamycin has the IUPAC name (3S,6R,7E,9R, 10R, 12R, 14S, 15E, 17E, 19E,21S,23S,26R,27R,34aS)-9, 10, 12, 13, 14,21,22,23,24,25,26,27,32,33,34,34a-hexadecahydro-9,27-dihydroxy-3-[(1R)-2-[(1 S,3 R,4R)-4-hydroxy-3-methoxycyclohexyl]-1-methylethyl]-10,21-dimethoxy-6,8, 12, 14,20,26-hexamethyl-23,27-epoxy-3H-pyrido[2, 1-c][1,4]-oxaazacyclohentriacontine-1,5, 11,28,29(4H,6H,31H)-pentone. Rapamycin has the CAS number 53123-88-9. Rapamycin may be produced synthetically (e.g., by chemical synthesis) or through use of a production method that does not include use of Streptomyces hygroscopicus.

“Analog” is used in accordance with its plain ordinary meaning within chemistry and biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof.

The term “rapamycin analog” or “rapalog” refers to an analog or derivative (e.g., a prodrug) of rapamycin.

The terms “active site mTOR inhibitor” and “ATP mimetic” refers to a compound that inhibits the activity of mTOR (e.g., kinase activity) and binds to the active site of mTOR (e.g., the ATP binding site, overlapping with the ATP binding site, blocking access by ATP to the ATP binding site of mTOR). Examples of active site mTOR inhibitors include, but are not limited to, FNK128, PP242, PP121, MLN0128, AZD8055, AZD2014, NVP-BEZ235, BGT226, SF1126, Torin 1, Torin 2, WYE 687, WYE 687 salt (e.g., hydrochloride), PF04691502, PI-103, CC-223, OSI-027, XL388, KU-0063794, GDC-0349, and PKI-587. In embodiments, an active site mTOR inhibitor is an asTORi. In some embodiments, “active site inhibitor” may refer to “active site mTOR inhibitor.”

The term “FKBP” refers to the protein Peptidyl-prolyl cis-trans isomerase. For non-limiting examples of FKBP, see Cell Mol Life Sci. 2013 September; 70(18):3243-75. In embodiments, “FKBP” may refer to “FKBP-12” or “FKBP 12” or “FKBP 1 A.” In embodiments, “FKBP” may refer to the human protein. Included in the term “FKBP” is the wildtype and mutant forms of the protein. In embodiments, “FKBP” may refer to the wildtype human protein. In embodiments, “FKBP” may refer to the wildtype human nucleic acid. In embodiments, the FKBP is a mutant FKBP. In embodiments, the mutant FKBP is associated with a disease that is not associated with wildtype FKBP. In embodiments, the FKBP includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to wildtype FKBP.

The term “FKBP-12” or “FKBP 12” or “FKBP1A” may refer to the protein “Peptidyl-prolyl cis-trans isomerase FKBP 1 A.” In embodiments, “FKBP-12” or “FKBP 12” or “FKBP 1 A” may refer to the human protein. Included in the term “FKBP-12” or “FKBP 12” or “FKBP 1 A” are the wildtype and mutant forms of the protein. In embodiments, “FKBP-12” or “FKBP 12” or “FKBP 1 A” may refer to the protein associated with Entrez Gene 2280, OMIM 186945, UniProt P62942, and/or RefSeq (protein) NP_000792 (SEQ ID NO:3). In embodiments, the reference numbers immediately above may refer to the protein, and associated nucleic acids, known as of the date of filing of this application. In embodiments, “FKBP-12” or “FKBP 12” or “FKBP 1 A” may refer to the wildtype human protein. In embodiments, “FKBP-12” or “FKBP 12” or “FKBP1A” may refer to the wildtype human nucleic acid. In embodiments, the FKBP-12 is a mutant FKBP-12. In embodiments, the mutant FKBP-12 is associated with a disease that is not associated with wildtype FKBP-12. In embodiments, the FKBP-12 may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to wildtype FKBP-12. In embodiments, the FKBP-12 has the protein sequence corresponding to reference number GI:206725550. In embodiments, the FKBP-12 has the protein sequence corresponding to RefSeq NP_000792.1 (SEQ ID NO:3).

The term “4E-BP1” or “4EBP1” or “EIF4EBP1” refers to the protein “Eukaryotic translation initiation factor 4E-binding protein 1.” In embodiments, “4E-BP1” or “4EBP1” or “EIF4EBP 1” may refer to the human protein. Included in the term “4E-BP 1” or “4EBP 1” or “EIF4EBP1” are the wildtype and mutant forms of the protein. In embodiments, “4E-BP1” or “4EBP1” or “EIF4EBP1” may refer to the protein associated with Entrez Gene 1978, OMIM 602223, UniProt Q13541, and/or RefSeq (protein) NP_004086 (SEQ ID NO:4). In embodiments, the reference numbers immediately above may refer to the protein, and associated nucleic acids, known as of the date of filing of this application. In embodiments, “4E-BP 1” or “4EBP1” or “EIF4EBP1” may refer to the wildtype human protein. In embodiments, “4E-BP1” or “4EBP1” or “EIF4EBP1” may refer to the wildtype human nucleic acid. In embodiments, the 4EBP1 is a mutant 4EBP1. In embodiments, the mutant 4EBP1 is associated with a disease that is not associated with wildtype 4EBP1. In embodiments, the 4EBP1 may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to wildtype 4EBP1. In embodiments, the 4EBP1 has the protein sequence corresponding to reference number GL4758258. In embodiments, the 4EBP1 has the protein sequence corresponding to RefSeq NP_004086.1 (SEQ ID NO:4).

The term “Akt” refers to the serine/threonine specific protein kinase involved in cellular processes such as glucose metabolism, apoptosis, proliferation, and other functions, also known as “protein kinase B” (PKB) or “Akt1.” In embodiments, “Akt” or “AM” or “PKB” may refer to the human protein. Included in the term “Akt” or “Akt1” or “PKB” are the wildtype and mutant forms of the protein. In embodiments, “Akt” or “Akt1” or “PKB” may refer to the protein associated with Entrez Gene 207, OMIM 164730, UniProt P31749, and/or RefSeq (protein) NP_005154 (SEQ ID NO:5). In embodiments, the reference numbers immediately above may refer to the protein, and associated nucleic acids, known as of the date of filing of this application. In embodiments, “Akt” or “Akt1” or “PKB” may refer to the wildtype human protein. In embodiments, “Akt” or “Akt1” or “PKB” may refer to the wildtype human nucleic acid. In embodiments, the Akt is a mutant Akt. In embodiments, the mutant Akt is associated with a disease that is not associated with wildtype Akt. In embodiments, the Akt may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations, or any range derivable therein) compared to wildtype Akt. In embodiments, the Akt has the protein sequence corresponding to reference number GI: 62241011. In embodiments, the Akt has the protein sequence corresponding to RefSeq NP_005154.2 (SEQ ID NO:5).

The present disclosure provides a method of treating a disease or disorder mediated by mTOR comprising administering to the subject suffering from or susceptible to developing a disease or disorder mediated by mTOR a therapeutically effective amount of one or more disclosed compositions or compounds. The present disclosure provides a method of preventing a disease or disorder mediated by mTOR comprising administering to the subject suffering from or susceptible to developing a disease or disorder mediated by mTOR a therapeutically effective amount of one or more disclosed compositions or compounds. The present disclosure provides a method of reducing the risk of a disease or disorder mediated by mTOR comprising administering to the subject suffering from or susceptible to developing a disease or disorder mediated by mTOR a therapeutically effective amount of one or more disclosed compositions or compounds.

In some embodiments, the disease is cancer or an immune-mediated disease. In some embodiments, the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors. In some embodiments, the disorder is liver cirrhosis. In some embodiments, the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis. In certain embodiments, the disease is tuberous sclerosis complex (TSC). In certain embodiments, the disease is pancreatic neuroendocrine tumor (PNET), mantle cell lymphoma (MCL), colorectal cancer or bowel cancer (CRC), uterine cancer, ovarian cancer, bladder cancer, genitourinary tract cancer, or renal cell carcinoma (RCC).

The present disclosure provides a method of treating cancer comprising administering to the subject a therapeutically effective amount of one or more disclosed compositions or compounds. In some embodiments, the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors. In some embodiments, the disorder is liver cirrhosis. In certain embodiments, the disease is tuberous sclerosis complex (TSC). In certain embodiments, the disease is pancreatic neuroendocrine tumor (PNET), mantle cell lymphoma (MCL), colorectal cancer or bowel cancer (CRC), uterine cancer, ovarian cancer, bladder cancer, genitourinary tract cancer, or renal cell carcinoma (RCC).

In certain embodiments, cancer includes human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AIL, ALL, and CML), or multiple myeloma. In certain embodiments, the disease is multiple myeloma. In certain embodiments, the disease is breast cancer. In certain embodiments, the disease is triple negative breast cancer.

In certain embodiments, cancer includes cancer, neoplasm or malignant tumors found in mammals (e.g. humans), including leukemia, carcinomas and sarcomas. Exemplary cancers that may be treated with a compound or method provided herein include cancer of the prostate, thyroid, endocrine system, brain, breast, cervix, colon, head and neck, liver, kidney, lung, non-small cell lung, melanoma, mesothelioma, ovary, sarcoma, stomach, uterus, Medulloblastoma, colorectal cancer, pancreatic cancer. Additional examples may include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In certain embodiments, the disease is leukemia. The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of aberrant cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

In certain embodiments, the disease is sarcoma. The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

In certain embodiments, the disease is melanoma. The term “melanoma” is taken to mean a tumor arising from the melanocyte system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

In certain embodiments, the disease is carcinoma. The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The present disclosure provides a method of treating an immune-mediated disease comprising administering to the subject a therapeutically effective amount of one or more disclosed compositions or compounds. In some embodiments, the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

In certain embodiments, the disease is autoimmune disease. As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressier's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

The present disclosure provide a method of treating an age related condition comprising administering to the subject a therapeutically effective amount of one or more disclosed compositions or compounds. In certain embodiments, the age related condition is selected from sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes.

In certain embodiments, the disclosed compositions or compounds can be used with regard to immunosenescence. Immunosenescence may refer to a decrease in immune function resulting in impaired immune response, e.g., to cancer, vaccination, infectious pathogens, among others. It involves both the host's capacity to respond to infections and the development of long-term immune memory, especially by vaccination. This immune deficiency is ubiquitous and found in both long- and short-lived species as a function of their age relative to life expectancy rather than chronological time. It is considered a major contributory factor to the increased frequency of morbidity and mortality among the elderly. Immunosenescence is not a random deteriorative phenomenon, rather it appears to inversely repeat an evolutionary pattern and most of the parameters affected by immunosenescence appear to be under genetic control. Immunosenescence can also be sometimes envisaged as the result of the continuous challenge of the unavoidable exposure to a variety of antigens such as viruses and bacteria. Immunosenescence is a multifactorial condition leading to many pathologically significant health problems, e.g., in the aged population. Age-dependent biological changes such as depletion of hematopoietic stem cells, an increase in PD1+ lymphocytes, a decline in the total number of phagocytes and NK cells and a decline in humoral immunity contribute to the onset of immunosenescence. In one aspect, immunosenescence can be measured in an individual by measuring telomere length in immune cells (See, e.g., U.S. Pat. No. 5,741,677). Immunosenescence can also be determined by documenting in an individual a lower than normal number of naive CD4 and/or CD8 T cells, T cell repertoire, the number of PD1-expressing T cells, e.g., a lower than normal number of PD-1 negative T cells, or response to vaccination in a subject greater than or equal to 65 years of age. In certain embodiments, mTOR selective modulation of certain T-cell populations may improve vaccine efficacy in the aging population and enhance effectiveness of cancer immunotherapy. The present disclosure provides a method of treating immunosenescence comprising administering to the subject a therapeutically effective amount of one or more disclosed compositions or compounds.

In certain embodiments, a disease that may be treated with a compound, pharmaceutical composition, or method described herein is organ or tissue transplant rejection (e.g. heart, lung, combined heart-lung, liver, kidney, pancreatic, skin or corneal transplants; graft-versus-host disease), restenosis, Hamartoma syndromes (e.g., tuberous sclerosis or Cowden Disease), Lymphangioleiomyomatosis, Retinitis pigmentosis, encephalomyelitis, insulin-dependent diabetes mellitus, lupus, dermatomyositis, arthritis, rheumatic diseases, Steroid-resistant acute Lymphoblastic Leukemia, fibrosis, scleroderma, pulmonary fibrosis, renal fibrosis, cystic fibrosis, Pulmonary hypertension, Multiple sclerosis, VHL syndrome, Carney complex, Familial adenonamtous polyposis, Juvenile polyposis syndrome, Birt-Hogg-Duke syndrome, Familial hypertrophic cardiomyopathy, Wolf-Parkinson-White syndrome, Parkinson's disease, Huntingtin's disease, Alzheimer's disease, dementias caused by tau mutations, spinocerebellar ataxia type 3, motor neuron disease caused by SOD1 mutations, neuronal ceroid lipofucinoses/Batten disease (pediatric neurodegeneration), wet macular degeneration, dry macular degeneration, muscle wasting (atrophy, cachexia), myopathies (e.g., Danon's disease), bacterial infection, viral infection, M. tuberculosis, group A streptococcus, HSV type I, HIV infection, Neurofibromatosis (e.g., Neurofibromatosis type 1), or Peutz-Jeghers syndrome.

In certain embodiments, the disease is neurodegenerative disease. As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

In certain embodiments, the disease is metabolic disease. As used herein, the term “metabolic disease” refers to a disease or condition in which a subject's metabolism or metabolic system (e.g., function of storing or utilizing energy) becomes impaired. Examples of metabolic diseases that may be treated with a compound, pharmaceutical composition, or method described herein include diabetes (e.g., type I or type II), obesity, metabolic syndrome, or a mitochondrial disease (e.g., dysfunction of mitochondria or aberrant mitochondrial function).

In certain embodiments, the disease is fungal disease. As used herein, the term “fungal disease” refers to a disease or condition associated with a fungus infection of the subject. Examples of fungal diseases that may be treated with a compound, pharmaceutical composition, or method described herein include infection with Mucor circinelloides, zygomycetes, Cryptococcus neoformans, Candida albicans, yeast, and Saccharomyces cerevisiae among others.

In certain embodiments, the disease is inflammatory disease. As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

In certain embodiments, the disease is cardiovascular disease. As used herein, the term “cardiovascular disease” refers to a disease or condition in which the function of a subject's cardiovascular system becomes impaired. Examples of cardiovascular diseases that may be treated with a compound, pharmaceutical composition, or method described herein include congestive heart failure; arrhythmogenic syndromes (e.g., paroxysomal tachycardia, delayed after depolarizations, ventricular tachycardia, sudden tachycardia, exercise-induced arrhythmias, long QT syndromes, or bidirectional tachycardia); thromboembolic disorders (e.g., arterial cardiovascular thromboembolic disorders, venous cardiovascular thromboembolic disorders, or thromboembolic disorders in the chambers of the heart); atherosclerosis; restenosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); myocardial infarction (first or recurrent); acute myocardial infarction (AMI); myocardial infarction; non-Q wave myocardial infarction; non-STE myocardial infarction; coronary artery disease; ischemic heart disease; cardiac ischemia; ischemia; ischemic sudden death; transient ischemic attack; stroke; peripheral occlusive arterial disease; venous thrombosis; deep vein thrombosis; thrombophlebitis; arterial embolism; coronary arterial thrombosis; cerebral arterial thrombosis, cerebral embolism; kidney embolism; pulmonary embolism; thrombosis (e.g., associated with prosthetic valves or other implants, indwelling catheters, stents, cardiopulmonary bypass, hemodialysis); thrombosis (e.g., associated with atherosclerosis, surgery, prolonged immobilization, arterial fibrillation, congenital thrombophilia, cancer, diabetes, hormones, or pregnancy); or cardiac arrhythmias (e.g., supraventricular arrhythmias, atrial arrhythmias, atrial flutter, or atrial fibrillation).

In an aspect is provided a method of treating a disease associated with an aberrant level of mTOR activity in a subject in need of such treatment. The disease may be caused by an upregulation of mTOR. The method may include administering to the subject one or more compositions or compounds described herein. The method may include administering to the subject a therapeutically effective amount of one or more compositions or compounds described herein (e.g., an mTOR modulator (e.g., inhibitor) as described above).

In an aspect is provided one or more compositions or compounds as described herein for use as a medicament. In embodiments, the medicament is useful for treating a disease caused by an upregulation of mTOR. The use may include administering to the subject one or more compositions or compounds described herein. The use may include administering to the subject a therapeutically effective amount of one or more compositions or compounds described herein (e.g., an mTOR modulator (e.g., inhibitor) as described above).

In an aspect is provided one or more compositions or compounds as described herein for use in the treatment of a disease caused by aberrant levels of mTORC1 activity in a subject in need of such treatment. The disease may be caused by an upregulation of mTORC1. The use may include administering to the subject one or more compositions or compounds described herein. The use may include administering to the subject a therapeutically effective amount of one or more compositions or compounds described herein (e.g., an mTORC1 modulator (e.g., inhibitor) as described above).

Upregulation of mTOR can result in an increased amount of mTOR activity compared to normal levels of mTOR activity in a particular subject or a population of healthy subjects. The increased amount of mTOR activity may result in, for example, excessive amounts of cell proliferation thereby causing the disease state.

The subject of treatment for the disease is typically a mammal. The mammal treated with the compound (e.g., compound described herein, mTOR modulator (e.g., inhibitor)) may be a human, nonhuman primate, and/or non-human mammal (e.g., rodent, canine).

In another aspect is provided a method of treating an mTOR activity-associated disease in a subject in need of such treatment, the method including administering one or more compositions or compounds as described herein, including embodiments (e.g., a claim, embodiment, example, table, figure, or claim) to the subject.

In another aspect is provided one or more compositions or compounds as described herein for use as a medicament. In embodiments, the medicament may be useful for treating an mTORC1 activity-associated disease in a subject in need of such treatment. In embodiments, the use may include administering one or more compositions or compounds as described herein, including embodiments (e.g., an aspect, embodiment, example, table, figure, or claim) to the subject.

In another aspect is provided one or more compositions or compounds for use in the treatment of an mTOR activity-associated disease in a subject in need of such treatment. In embodiments, the use may include administering one or more compositions or compounds as described herein, including embodiments (e.g., an aspect, embodiment, example, table, figure, or claim) to the subject.

In embodiments, the mTOR activity-associated disease or disease associated with aberrant levels of mTOR activity is cancer. In embodiments, the mTOR activity-associated disease or disease associated with aberrant levels of mTOR activity is an autoimmune disease. In embodiments, the mTOR activity-associated disease or disease associated with aberrant levels of mTOR activity is an inflammatory disease. In embodiments, the mTOR activity-associated disease or disease associated with aberrant levels of mTOR activity is a neurodegenerative disease. In embodiments, the mTOR activity-associated disease or disease associated with aberrant levels of mTOR activity is a metabolic disease. In embodiments, the mTOR activity-associated disease or disease associated with aberrant levels of mTOR activity is transplant rejection. In embodiments, the mTOR activity-associated disease or disease associated with aberrant levels of mTOR activity is fungal infection. In embodiments, the mTOR activity-associated disease or disease associated with aberrant levels of mTOR activity is a cardiovascular disease.

In embodiments, the mTOR activity-associated disease or disease associated with aberrant levels of mTOR activity is aging. In embodiments, the mTOR activity-associated disease or disease associated with aberrant levels of mTOR activity is dying of an age-related disease. In embodiments, the mTOR activity-associated disease or disease associated with aberrant levels of mTOR activity is an age-related condition. In certain embodiments, the age related condition is selected from the group consisting of sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes. In certain embodiments, mTOR selective modulation of certain T-cell populations may improve vaccine efficacy in the aging population and enhance effectiveness of cancer immunotherapy. The present disclosure provides a method of treating immunosenescence comprising administering to the subject a therapeutically effective amount of one or more disclosed compounds.

In embodiments, the mTOR activity-associated disease or disease associated with aberrant levels of mTOR activity is cancer (e.g., carcinomas, sarcomas, adenocarcinomas, lymphomas, leukemias, solid cancers, lymphoid cancers; cancer of the kidney, breast, lung, bladder, colon, gastrointestinal, ovarian, prostate, pancreas, stomach, brain, head and neck, skin, uterine, esophagus, liver; testicular cancer, glioma, hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), multiple myeloma, and breast cancer (e.g., triple negative breast cancer)).

In embodiments, the mTOR activity-associated disease or disease associated with aberrant levels of mTOR activity is Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressier's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatry Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, Wegener's granulomatosis (i.e., Granulomatosis with Polyangiitis (GPA), traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, atopic dermatitis, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HTV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, diabetes (e.g., type I or type II), obesity, metabolic syndrome, a mitochondrial disease (e.g., dysfunction of mitochondria or aberrant mitochondrial function), fungal infection, transplant rejection, or a cardiovascular disease (e.g., congestive heart failure; arrhythmogenic syndromes (e.g., paroxysomal tachycardia, delayed after depolarizations, ventricular tachycardia, sudden tachycardia, exercise-induced arrhythmias, long QT syndromes, or bidirectional tachycardia); thromboembolic disorders (e.g., arterial cardiovascular thromboembolic disorders, venous cardiovascular thromboembolic disorders, or thromboembolic disorders in the chambers of the heart); atherosclerosis; restenosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); myocardial infarction (first or recurrent); acute myocardial infarction (AMI); myocardial infarction; non-Q wave myocardial infarction; non-STE myocardial infarction; coronary artery disease; ischemic heart disease; cardiac ischemia; ischemia; ischemic sudden death; transient ischemic attack; stroke; peripheral occlusive arterial disease; venous thrombosis; deep vein thrombosis; thrombophlebitis; arterial embolism; coronary arterial thrombosis; cerebral arterial thrombosis, cerebral embolism; kidney embolism; pulmonary embolism; thrombosis (e.g., associated with prosthetic valves or other implants, indwelling catheters, stents, cardiopulmonary bypass, hemodialysis); thrombosis (e.g., associated with atherosclerosis, surgery, prolonged immobilization, arterial fibrillation, congenital thrombophilia, cancer, diabetes, hormones, or pregnancy); or cardiac arrhythmias (e.g., supraventricular arrhythmias, atrial arrhythmias, atrial flutter, or atrial fibrillation).

In an aspect is provided a method of treating a disease including administering an effective amount of one or more compositions or compounds as described herein. In an aspect is provided one or more compositions or compounds as described herein for use as a medicament (e.g., for treatment of a disease). In an aspect is provided one or more compositions or compounds as described herein for use in the treatment of a disease (e.g., including administering an effective amount of one or more compositions or compounds as described herein). In embodiments, the disease is cancer. In embodiments, the disease is an autoimmune disease. In embodiments, the disease is an inflammatory disease. In embodiments, the disease is a neurodegenerative disease. In embodiments, the disease is a metabolic disease. In embodiments, the disease is fungal infection. In embodiments, the disease is transplant rejection. In embodiments, the disease is a cardiovascular disease.

In embodiments, the disease is cancer (e.g., carcinomas, sarcomas, adenocarcinomas, lymphomas, leukemias, solid cancers, lymphoid cancers; cancer of the kidney, breast, lung, bladder, colon, ovarian, prostate, pancreas, stomach, brain, head and neck, skin, uterine, esophagus, liver; testicular cancer, glioma, hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), multiple myeloma, and breast cancer (e.g., triple negative breast cancer)).

In embodiments, the disease is Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, Wegener's granulomatosis (i.e., Granulomatosis with Polyangiitis (GPA), traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, atopic dermatitis, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HTV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, diabetes (e.g., type I or type II), obesity, metabolic syndrome, a mitochondrial disease (e.g., dysfunction of mitochondria or aberrant mitochondrial function), fungal infection, transplant rejection, or a cardiovascular disease (e.g., congestive heart failure; arrhythmogenic syndromes (e.g., paroxysomal tachycardia, delayed after depolarizations, ventricular tachycardia, sudden tachycardia, exercise-induced arrhythmias, long QT syndromes, or bidirectional tachycardia); thromboembolic disorders (e.g., arterial cardiovascular thromboembolic disorders, venous cardiovascular thromboembolic disorders, or thromboembolic disorders in the chambers of the heart); atherosclerosis; restenosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); myocardial infarction (first or recurrent); acute myocardial infarction (AMI); myocardial infarction; non-Q wave myocardial infarction; non-STE myocardial infarction; coronary artery disease; ischemic heart disease; cardiac ischemia; ischemia; ischemic sudden death; transient ischemic attack; stroke; peripheral occlusive arterial disease; venous thrombosis; deep vein thrombosis; thrombophlebitis; arterial embolism; coronary arterial thrombosis; cerebral arterial thrombosis, cerebral embolism; kidney embolism; pulmonary embolism; thrombosis (e.g., associated with prosthetic valves or other implants, indwelling catheters, stents, cardiopulmonary bypass, hemodialysis); thrombosis (e.g., associated with atherosclerosis, surgery, prolonged immobilization, arterial fibrillation, congenital thrombophilia, cancer, diabetes, hormones, or pregnancy); or cardiac arrhythmias (e.g., supraventricular arrhythmias, atrial arrhythmias, atrial flutter, or atrial fibrillation). In embodiments, the disease is a polycystic disease. In embodiments, the disease is polycystic kidney disease. In embodiments, the disease is stenosis. In embodiments, the disease is restenosis. In embodiments, the disease is neointimal proliferation. In embodiments, the disease is neointimal hyperplasia.

In another aspect is provided a method of treating aging in a subject in need of such treatment, the method including administering one or more compositions or compounds as described herein, including embodiments (e.g., a claim, embodiment, example, table, figure, or claim) to the subject. The present disclosure provides a method of treating immunosenescence comprising administering to the subject a therapeutically effective amount of one or more disclosed compounds or compositions.

In another aspect is provided one or more compositions or compounds as described herein for use as a medicament. In embodiments, the medicament may be useful for treating aging in a subject in need of such treatment. In embodiments, the use may include administering one or more compositions or compounds as described herein, including embodiments (e.g., an aspect, embodiment, example, table, figure, or claim) to the subject.

In another aspect is provided one or more compositions or compounds disclosed herein for use in the treatment of aging in a subject in need of such treatment. In embodiments, the use may include administering one or more compositions or compounds as described herein, including embodiments (e.g., an aspect, embodiment, example, table, figure, or claim) to the subject.

In another aspect is provided a method of extending life span or inducing longevity in a subject in need of such treatment, the method including administering one or more compositions or compounds as described herein, including embodiments (e.g., a claim, embodiment, example, table, figure, or claim) to the subject.

In another aspect is provided one or more compositions or compounds as described herein for use as a medicament. In embodiments, the medicament may be useful for extending life span or inducing longevity in a subject in need of such treatment. In embodiments, the use may include administering one or more compositions or compounds as described herein, including embodiments (e.g., an aspect, embodiment, example, table, figure, or claim) to the subject.

In another aspect is provided one or more compositions or compounds for use in extending life span or inducing longevity in a subject in need of such treatment. In embodiments, the use may include administering one or more compositions or compounds as described herein, including embodiments (e.g., an aspect, embodiment, example, table, figure, or claim) to the subject.

In an aspect is provided a method of treating a polycystic disease in a subject in need of such treatment. The polycystic disease may be polycystic kidney disease. The method may include administering to the subject one or more compositions or compounds described herein. The method may include administering to the subject a therapeutically effective amount of one or more compositions or compounds described herein (e.g., an mTOR modulator (e.g., inhibitor) as described above).

In an aspect is provided one or more compositions or compounds as described herein for use as a medicament. In embodiments, the medicament is useful for treating a polycystic disease. The polycystic disease may be polycystic kidney disease. The use may include administering to the subject one or more compositions or compounds described herein. The use may include administering to the subject a therapeutically effective amount of one or more compositions or compounds described herein (e.g., an mTOR modulator (e.g., inhibitor) as described above).

In an aspect is provided one or more compositions or compounds as described herein for use in the treatment of a polycystic disease in a subject in need of such treatment. The polycystic disease may be polycystic kidney disease. The use may include administering to the subject one or more compositions or compounds described herein. The use may include administering to the subject a therapeutically effective amount of one or more compositions or compounds described herein (e.g., an mTOR modulator (e.g., inhibitor) as described above).

In an aspect is provided a method of treating stenosis in a subject in need of such treatment. The stenosis may be restenosis. The method may include administering to the subject one or more compositions or compounds described herein. In embodiments the one or more compositions or compounds are administered in a drug eluting stent. The method may include administering to the subject a therapeutically effective amount of one or more compositions or compounds described herein (e.g., an mTOR modulator (e.g., inhibitor) as described above).

In an aspect is provided one or more compositions or compounds as described herein for use as a medicament. In embodiments, the medicament is useful for treating stenosis. The stenosis may be restenosis. The use may include administering to the subject one or more compositions or compounds described herein. In embodiments the compound is administered in a drug eluting stent. The use may include administering to the subject a therapeutically effective amount of one or more compositions or compounds described herein (e.g., an mTOR modulator (e.g., inhibitor) as described above).

In an aspect is provided one or more compositions or compounds as described herein for use in the treatment of stenosis in a subject in need of such treatment. The stenosis may be restenosis. The use may include administering to the subject one or more compositions or compounds described herein. In embodiments the one or more compositions or compounds are administered in a drug eluting stent. The use may include administering to the subject a therapeutically effective amount of one or more compositions or compounds described herein (e.g., an mTOR modulator (e.g., inhibitor) as described above).

In embodiments, the disease is a disease described herein and the compound is a compound described herein and the composition is a composition described herein.

### Methods of Modulating mTOR

In some embodiments, compounds disclosed herein are more selective inhibitors of mTORC1 versus mTORC2. In some embodiments, compounds disclosed herein are more selective inhibitors of mTORC2 versus mTORC1. In some embodiments, compounds disclosed herein exhibit no selectivity difference between mTORC1 and mTORC2.

In another aspect is provided a method of modulating mTORC1 activity in a subject in need thereof, including administering to the subject an effective amount of a compound as described herein, or a pharmaceutically acceptable salt thereof. In embodiments, the method includes inhibiting mTORC1 activity. In embodiments, the method includes inhibiting mTORC1 activity and not inhibiting mTORC2 activity.

In embodiments, the method includes inhibiting mTORC1 activity more than inhibiting mTORC2 activity. In embodiments, the method includes inhibiting mTORC1 activity at least 1.1 fold as much as inhibiting mTORC2 activity (e.g., at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 10000, 20000, 30000, 40000, 50000, 60000, 70000, 80000, 90000, 100000, 100000, 200000, 300000, 400000, 500000, 600000, 700000, 800000, 900000, or 1000000 fold).

In another aspect is provided a method of modulating mTORC2 activity in a subject in need thereof, including administering to the subject an effective amount of a compound as described herein, or a pharmaceutically acceptable salt thereof. In embodiments, the method includes inhibiting mTORC2 activity. In embodiments, the method includes inhibiting mTORC2 activity and not inhibiting mTORC1 activity.

In embodiments, the method includes inhibiting mTORC2 activity more than inhibiting mTORC1 activity. In embodiments, the method includes inhibiting mTORC2 activity at least 1.1 fold as much as inhibiting mTORC1 activity (e.g., at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 10000, 20000, 30000, 40000, 50000, 60000, 70000, 80000, 90000, 100000, 100000, 200000, 300000, 400000, 500000, 600000, 700000, 800000, 900000, or 1000000 fold).

In some embodiments, the mTOR is in a cell. In some embodiments, the cell is a mammalian cell, such as a human cell. The cell may be isolated in vitro, form part of a tissue in vitro, or may form part of an organism.

### EXEMPLARY EMBODIMENTS

Some embodiments of this disclosure are Embodiment I, as follows:

Embodiment I-1. A compound of Formula I:

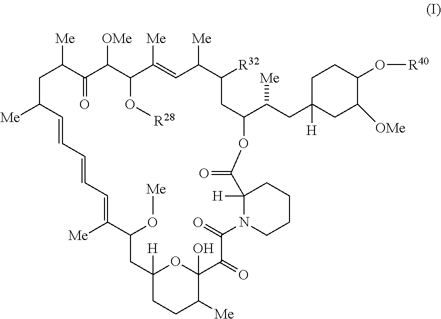

or a pharmaceutically acceptable salt or tautomer thereof, wherein:
R³² is —H, ═O or —OR³;
R²⁸ is —H or —C(═Z¹)—R^{28a}.
R⁴⁰ is —H or —C(═Z¹)—R^{40a};
   wherein at least one of R²⁸ and R⁴⁰ is not H;
Z¹ is O or S;
R^{28a} and R^{40a} are independently -A-L¹-A²-B; -A¹-A²-B; -L²-A¹-L¹-A²-L³-B; —O—(C₁-C₆)alkyl; or —O—(C₆-C₁₀)aryl; wherein the aryl is unsubstituted or substituted with 1-5 substituents selected from —NO₂ and halogen;
A¹ and A² are independently absent or are independently selected from

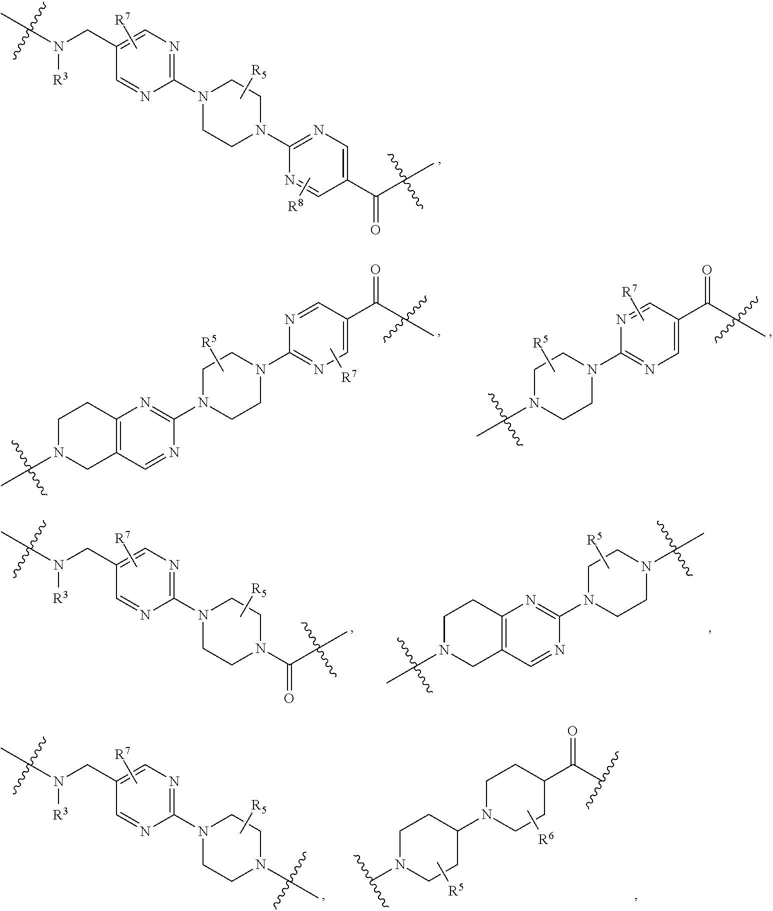

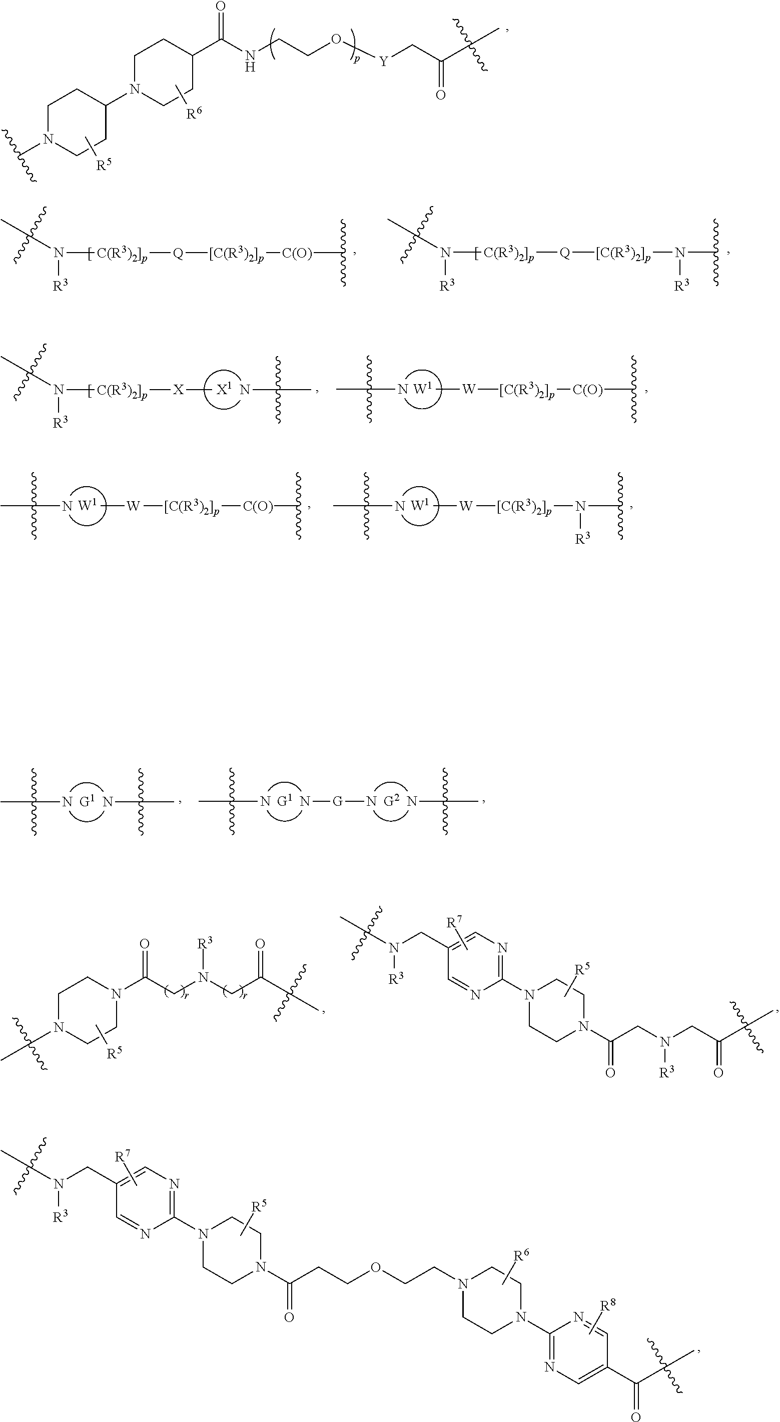

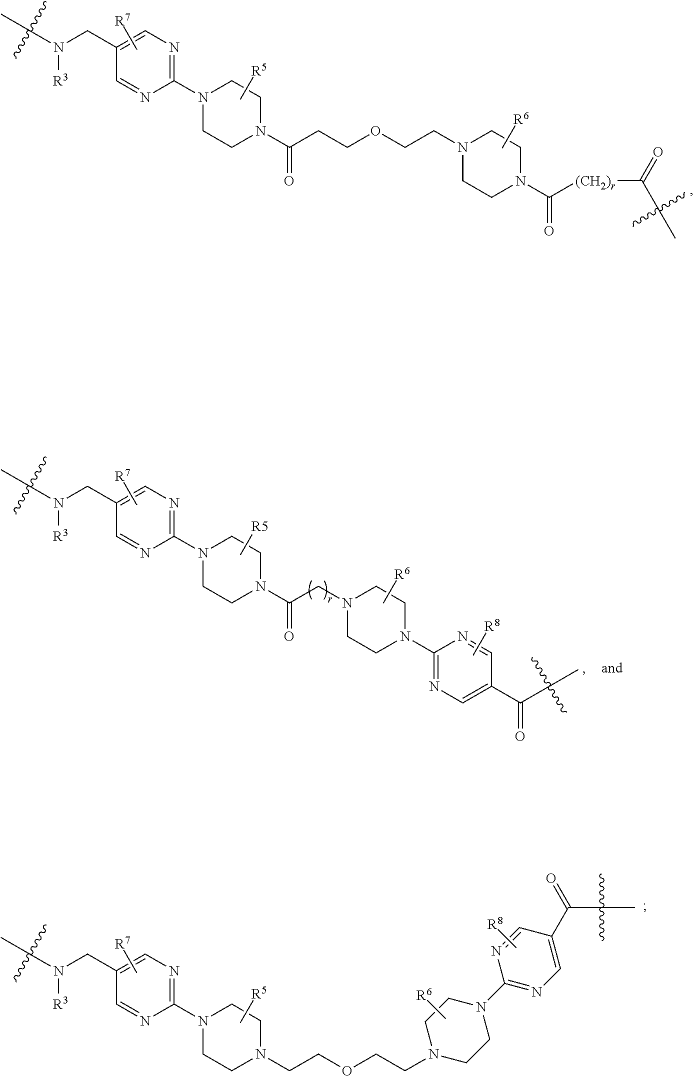

wherein the bond on the left side of A¹, as drawn, is bound to —C(═Z¹)— or L²; and wherein the bond on the right side of the A² moiety, as drawn, is bound to B or L³;
each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ is a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;
L¹ is selected from

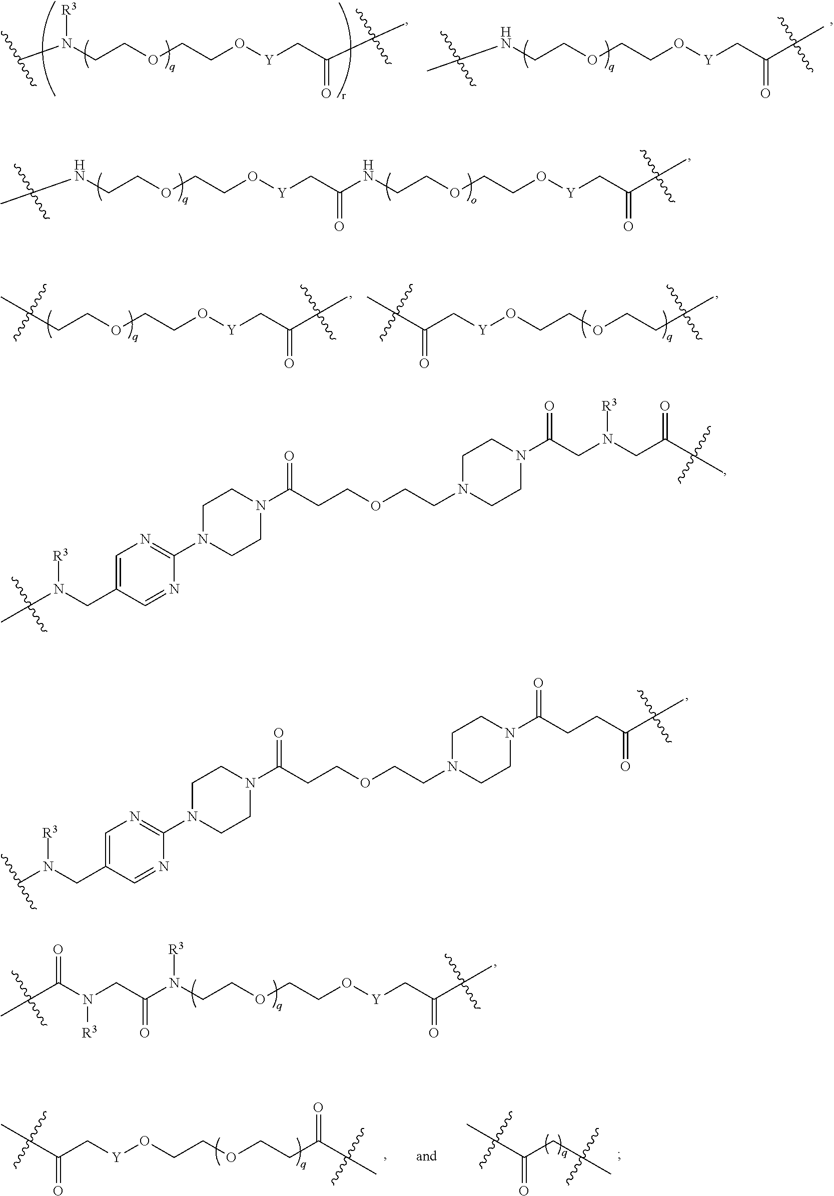

L² and L³ are independently absent or are independently selected from

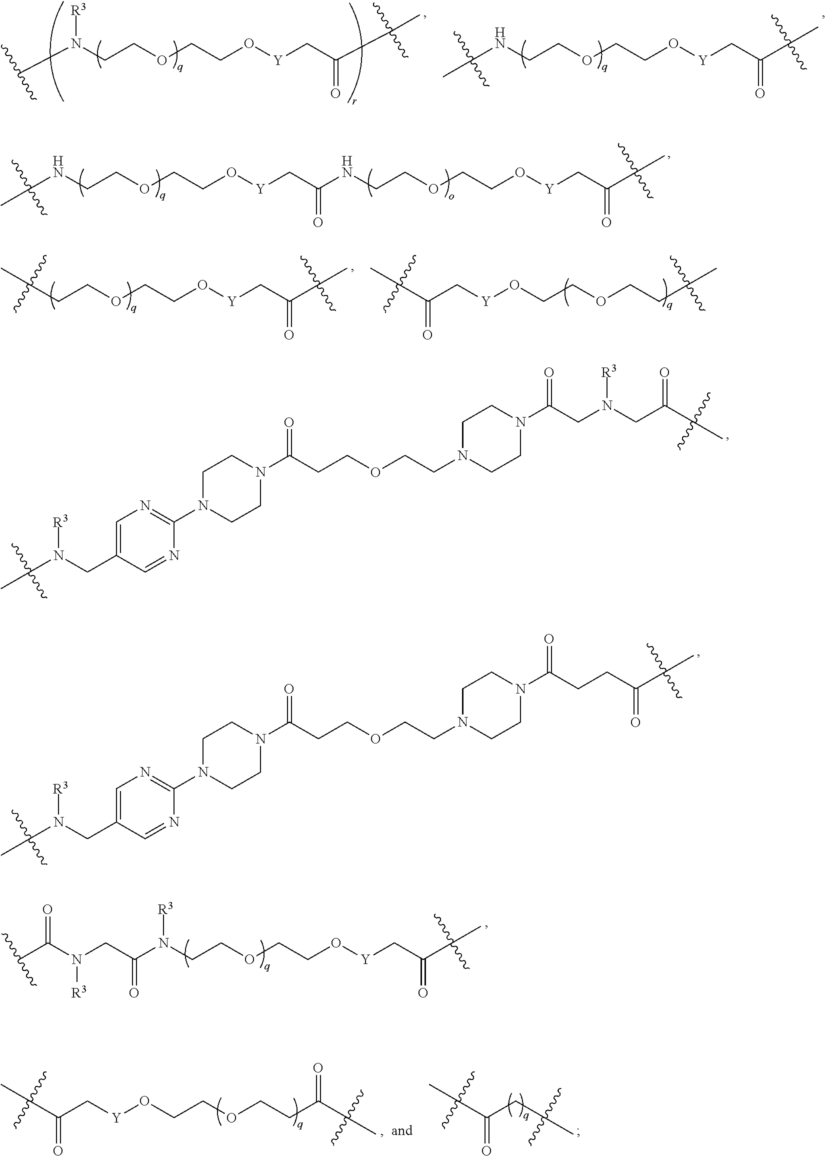

B is selected from

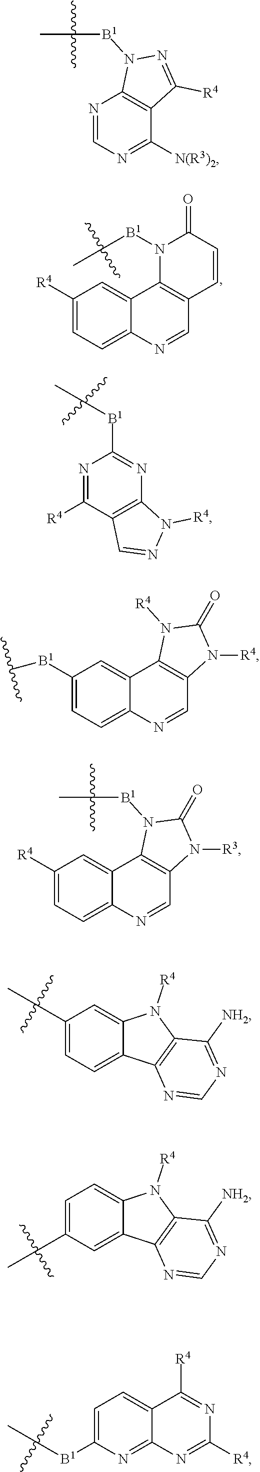

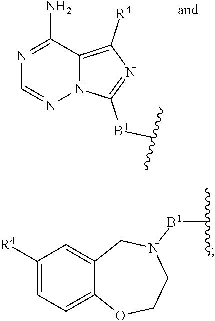

B¹ is selected from

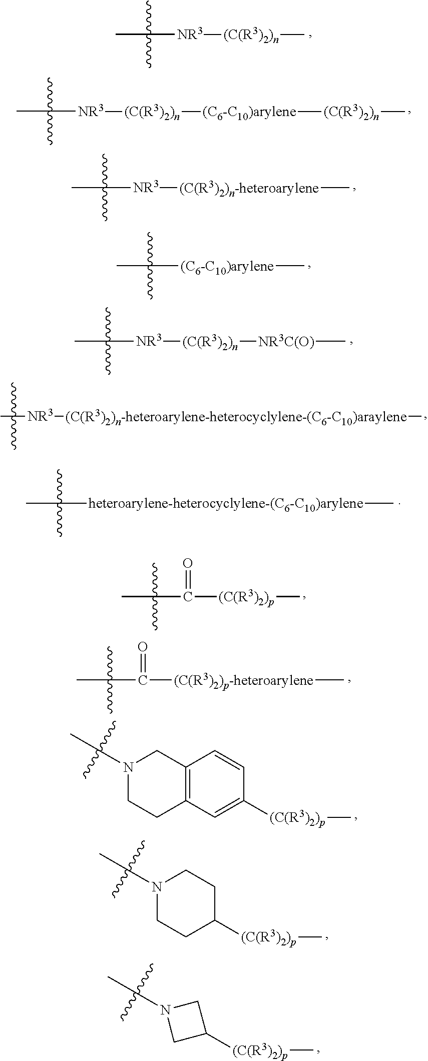

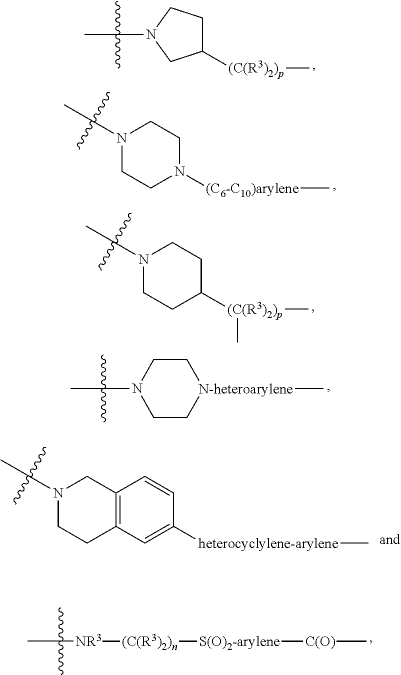

wherein the

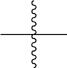

bond on the left side of B¹, as drawn, is bound to A², L³, or L¹; and wherein the heteroarylene, heterocyclylene, and arylene are optionally substituted with alkyl, hydroxyalkyl, haloalkyl, alkoxy, halogen, or hydroxyl;
each R³ is independently H or (C₁-C₆)alkyl;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl; or —C(O)NR³-heterocyclyl;
each R⁵ is independently H, (C₁-C₆)alkyl, —C(O)OR³, or —N(R³)₂, wherein the alkyl is optionally substituted with —N(R³)₂ or —OR³;
each R⁶ is independently H, (C₁-C₆)alkyl, —C(O)OR³, or —N(R³)₂, wherein the alkyl is optionally substituted with —N(R³)₂ or —OR³;
each R⁷ is independently H, (C₁-C₆)alkyl, —C(O)OR³, or —N(R³)₂, wherein the alkyl is optionally substituted with —N(R³)₂ or —OR³;
each R⁸ is independently H, (C₁-C₆)alkyl, —C(O)OR³, or —N(R³)₂, wherein the alkyl is optionally substituted with —N(R³)₂ or —OR³;
each Y is independently C(R³)₂ or a bond;
each n is independently a number from one to 12;
each o is independently a number from zero to 30;
each p is independently a number from zero to 12;
each q is independently a number from zero to 30; and
each r is independently a number from one to 6.

Embodiment I-2. A compound of Formula II:

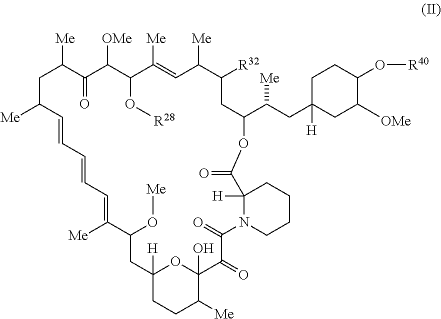

or a pharmaceutically acceptable salt or tautomer thereof, wherein:
R³² is —H, ═O or —OR³;
R²⁸ is —H or —C(═Z¹)—R^{28a}.
R⁴⁰ is —H or —C(═Z¹)—R^{40a};
   wherein at least one of R²⁸ and R⁴⁰ is not H;
Z¹ is O or S;
R^{28a} and R^{40a} are independently -A¹-L¹-A²-B; -A¹-A²-B; —O—(C₁-C₆)alkyl; or —O—(C₆-C₁₀)aryl; wherein the aryl is unsubstituted or substituted with 1-5 substituents selected from —NO₂ and halogen;
A¹ and A² are independently absent or are independently selected from

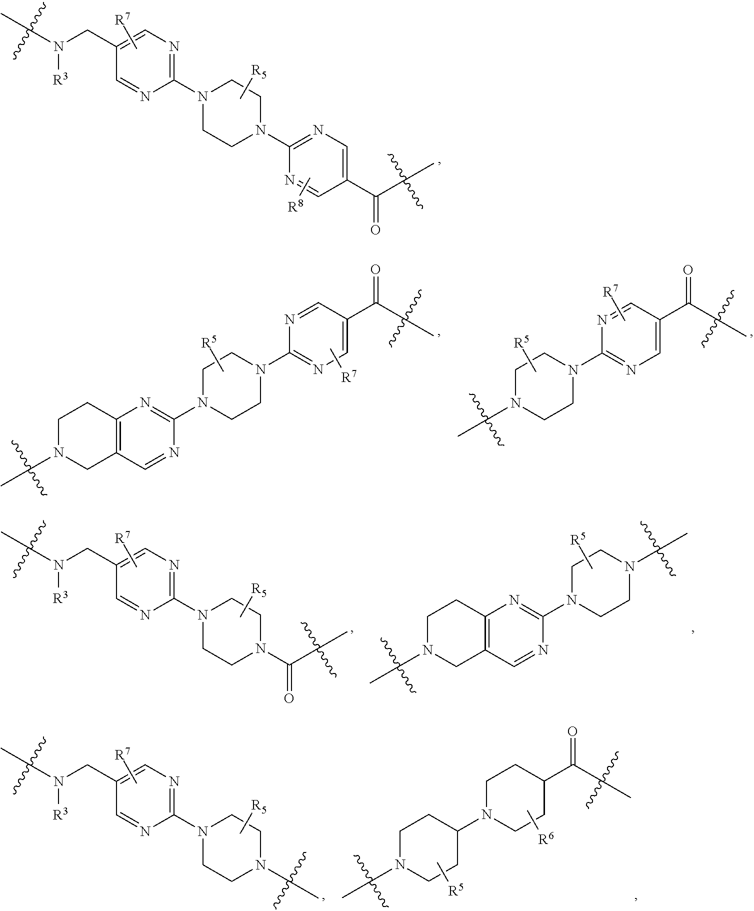

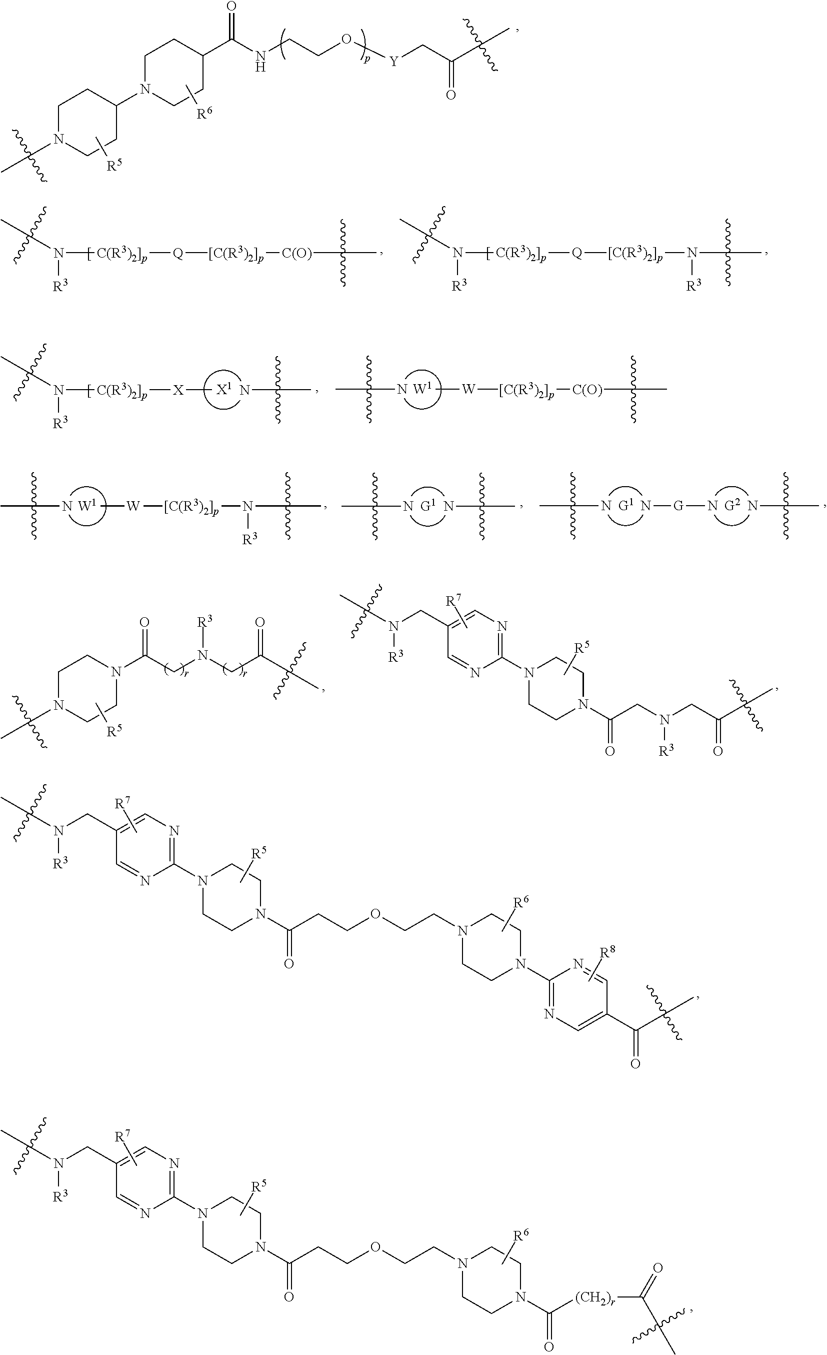

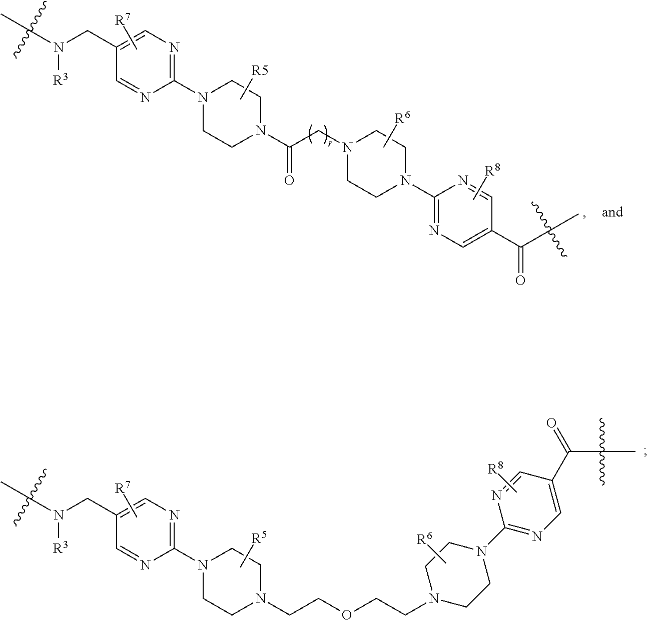

wherein the bond on the left side of A¹, as drawn, is bound to —C(═Z¹)—; and wherein the bond on the right side of the A² moiety, as drawn, is bound to B.
each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is a heteroarylene or heterocyclylene ring;
each W is independently absent or 1 to 2 rings selected from arylene, cycloalkylene, heteroarylene, and heterocyclylene;
each W¹ is a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;
L¹ is selected from

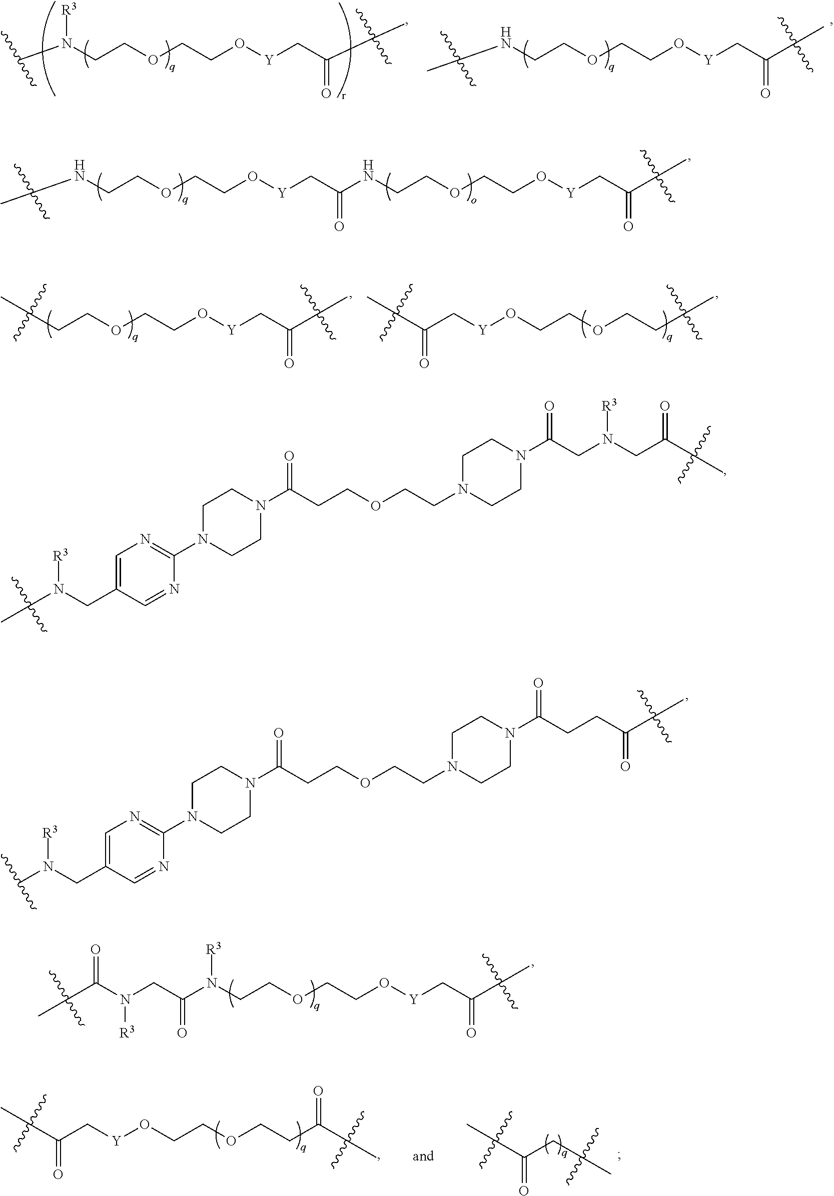

B is selected from

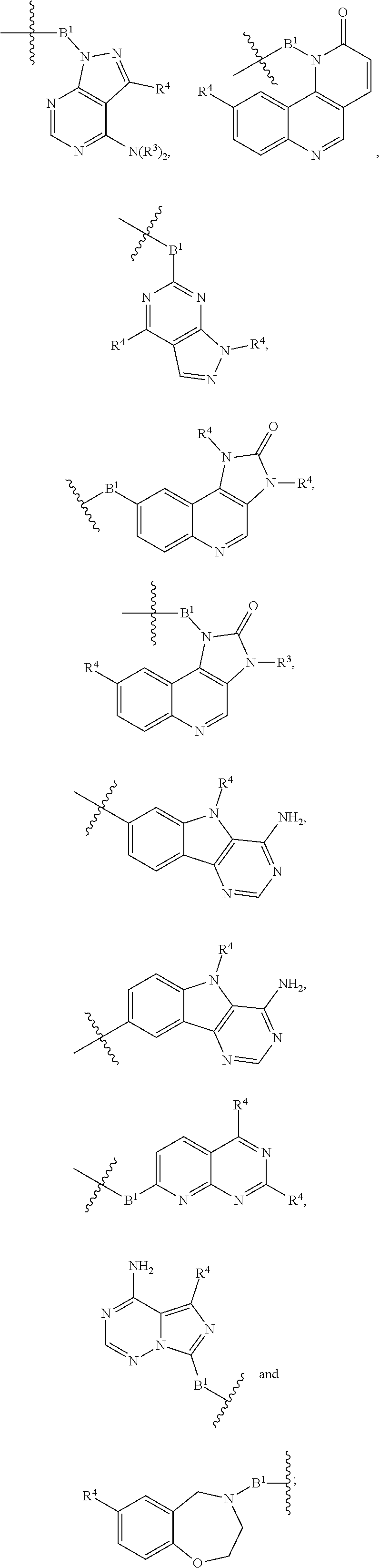

B¹ is selected from

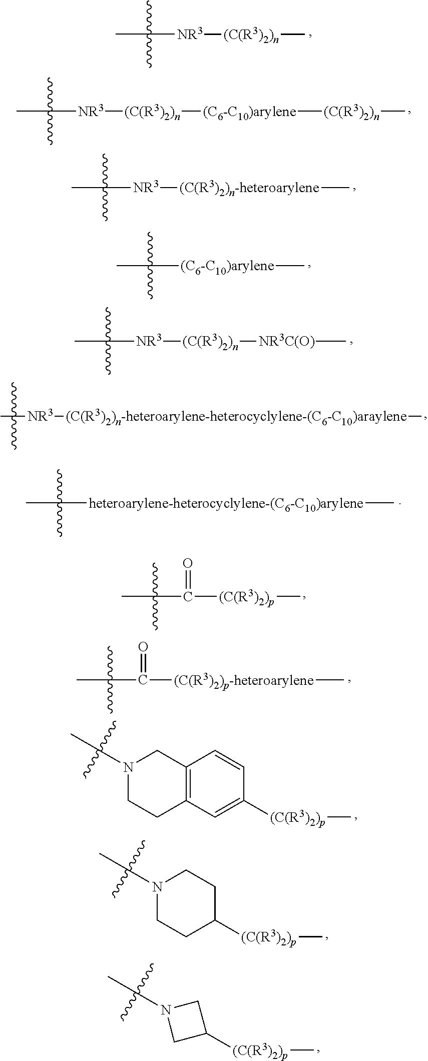

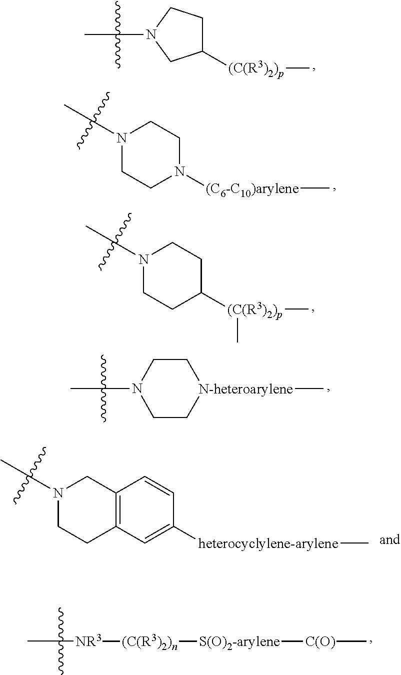

wherein the

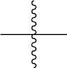

bond on the left side of B¹, as drawn, is bound to A² or L¹; and wherein the heteroarylene, heterocyclylene, and arylene are optionally substituted with alkyl, hydroxyalkyl, haloalkyl, alkoxy, halogen, or hydroxyl;
each R³ is independently H or (C₁-C₆)alkyl;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl; or —C(O)NR³-heterocyclyl;
each R⁵ is independently H, (C₁-C₆)alkyl, —C(O)OR³, or —N(R³)₂, wherein the alkyl is optionally substituted with —N(R³)₂ or —OR³;
each R⁶ is independently H, (C₁-C₆)alkyl, —C(O)OR³, or —N(R³)₂, wherein the alkyl is optionally substituted with —N(R³)₂ or —OR³;
each R⁷ is independently H, (C₁-C₆)alkyl, —C(O)OR³, or —N(R³)₂, wherein the alkyl is optionally substituted with —N(R³)₂ or —OR³;
each R⁸ is independently H, (C₁-C₆)alkyl, —C(O)OR³, or —N(R³)₂, wherein the alkyl is optionally substituted with —N(R³)₂ or —OR³;
each Y is independently C(R³)₂ or a bond;
each n is independently a number from one to 12;
each o is independently a number from zero to 30;
each p is independently a number from zero to 12;
each q is independently a number from zero to 30; and
each r is independently a number from one to 6.

Embodiment I-3. The compound of Embodiment I-1 or I-2, wherein R³² is ═O.

Embodiment I-4. The compound of Embodiment I-1 or I-2, wherein R³² is —OR³.

Embodiment I-5. The compound of any one of Embodiments I-1 to I-4, wherein the compounds are represented by the structure of Formula I-40:

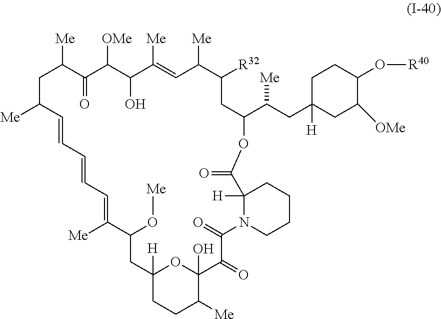

or a pharmaceutically acceptable salt or tautomer thereof.

Embodiment I-6. The compound of Embodiment I-5, wherein Z¹ is O.

Embodiment I-7. The compound of Embodiment I-5, wherein Z¹ is S.

Embodiment I-8. The compound of any one of Embodiments I-5 to I-7, wherein R^{40a} is -A¹-L¹-A²-B, wherein A¹ and A² are absent.

Embodiment I-9. The compound of any one of Embodiments I-5 to I-7, wherein R^{40a} is -A¹-L¹-A²-B, wherein A² is absent.

Embodiment I-10. The compound of any one of Embodiments I-5 to I-7, wherein R^{40a} is -A¹-L¹-A²-B, wherein A¹ is absent.

Embodiment I-11. The compound of any one of Embodiments I-5 to I-7, wherein R^{40a} is -A¹-L¹-A²-B.

Embodiment I-12. The compound of any one of Embodiments I-5 to I-7, wherein R^{40a} is -A¹-A²-B.

Embodiment I-13. The compound of any one of Embodiments I-5 to I-7, wherein R^{40a} is -L²-A¹-L¹-A²-L³-B, wherein L² and A¹ are absent.

Embodiment I-14. The compound of any one of Embodiments I-5 to I-7, wherein R^{40a} is -L²-A¹-L¹-A²-L³-B, wherein L² is absent.

Embodiment I-15. The compound of any one of Embodiments I-5 to I-7, wherein R^{40a} is -L²-A¹-L¹-A²-L³-B, wherein L³ is absent.

Embodiment I-16. The compound of any one of Embodiments I-5 to I-7, wherein R^{40a} is —O—(C₁-C₆)alkyl or —O—(C₆-C₁₀)aryl; wherein the aryl is unsubstituted or substituted with 1-5 substituents selected from —NO₂ and halogen.

Embodiment I-17. The compound of any one of Embodiments I-1 to I-4, wherein the compounds are represented by the structure of Formula I-28:

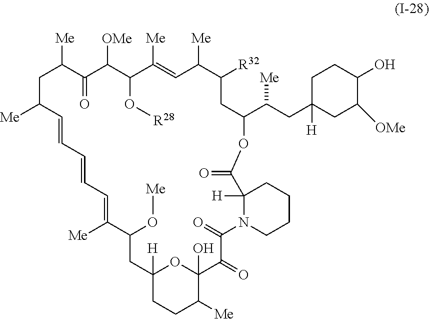

or a pharmaceutically acceptable salt or tautomer thereof.

Embodiment I-18. The compound of Embodiment I-17, wherein Z¹ is O.

Embodiment I-19. The compound of Embodiment I-17, wherein Z¹ is S.

Embodiment I-20. The compound of any one of Embodiments I-17 to I-19, wherein R^{28a} is -A¹-L¹-A²-B, wherein A¹ and A² are absent.

Embodiment I-21. The compound of any one of Embodiments I-17 to I-19, wherein R^{28a} is -A¹-L¹-A²-B, wherein A² is absent.

Embodiment I-22. The compound of any one of Embodiments I-17 to I-19, wherein R^{28a} is -A¹-L¹-A²-B, wherein A¹ is absent.

Embodiment I-23. The compound of any one of Embodiments I-17 to I-19, wherein R^{28a} is -A¹-L¹-A²-B.

Embodiment I-24. The compound of any one of Embodiments I-17 to I-19, wherein R^{28a} is -A¹-A²-B.

Embodiment I-25. The compound of any one of Embodiments I-17 to I-19, wherein R^{28a} is -L²-A¹-L¹-A²-L³-B, wherein L² and A¹ are absent.

Embodiment I-26. The compound of any one of Embodiments I-17 to I-19, wherein R^{28a} is -L²-A¹-L¹-A²-L³-B, wherein L² is absent.

Embodiment I-27. The compound of any one of Embodiments I-17 to I-19, wherein R^{28a} is -L²-A¹-L¹-A²-L³-B, wherein L³ is absent.

Embodiment I-28. The compound of any one of Embodiments I-17 to I-19, wherein R^{28a} is —O—(C₁-C₆)alkyl or —O—(C₆-C₁₀)aryl; wherein the aryl is unsubstituted or substituted with 1-5 substituents selected from —NO₂ and halogen.

Embodiment I-29. The compound of any one of Embodiments I-1 to I-11, I-13 to I-15, I-17 to I-23, and I-25 to I-27, wherein L¹ is

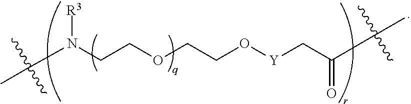

Embodiment I-30. The compound of any one of Embodiments I-1 to I-11, I-13 to I-15, I-17 to I-23, and I-25 to I-27, wherein L¹ is

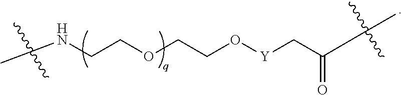

Embodiment I-31. The compound of any one of Embodiments I-1 to I-11, I-13 to I-15, I-17 to I-23, and I-25 to I-27, wherein L¹ is

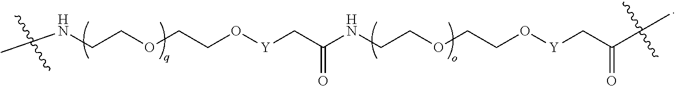

Embodiment I-32. The compound of any one of Embodiments I-1 to I-11, I-13 to I-15, I-17 to I-23, and I-25 to I-27, wherein L¹ is

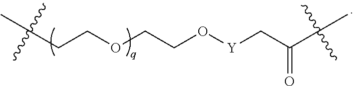

Embodiment I-33. The compound of any one of Embodiments I-1 to I-11, I-13 to I-15, I-17 to I-23, and I-25 to I-27, wherein L¹ is

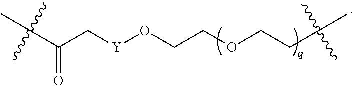

Embodiment I-34. The compound of any one of Embodiments I-1 to I-7, I-15, I-17 to I-19, and I-27, wherein L² is

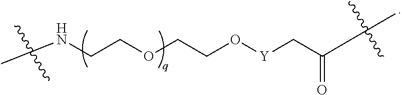

Embodiment I-35. The compound of any one of Embodiments I-1 to I-7, I-13 to I-14, I-17 to I-19, and I-25 to I-26, wherein L³ is

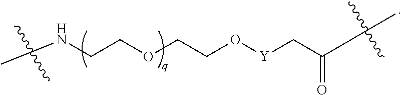

Embodiment I-36. The compound of any one of Embodiments I-1 to I-8, I-10, I-13, I-17 to I-19, I-20, I-22, I-25 and I-29 to I-35, wherein A¹ is absent.

Embodiment I-37. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

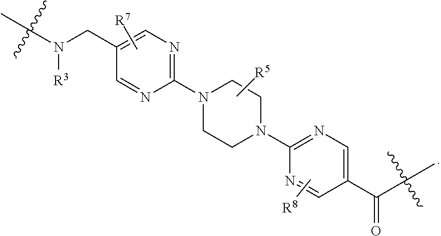

Embodiment I-38. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

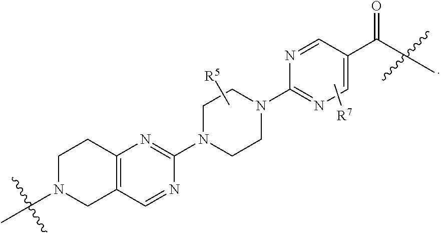

Embodiment I-39. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

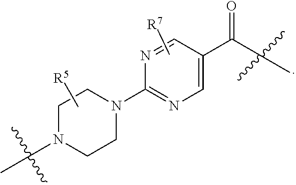

Embodiment I-40. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

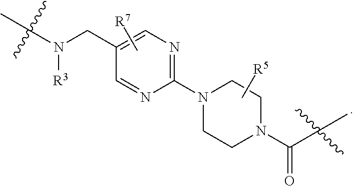

Embodiment I-41. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

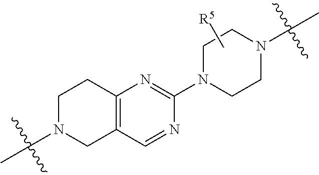

Embodiment I-42. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

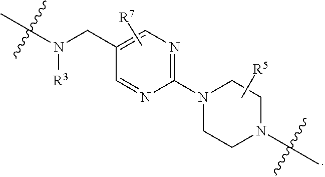

Embodiment I-43. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

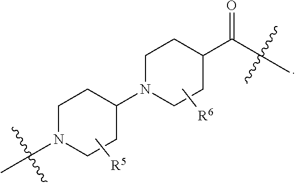

Embodiment I-44. The compound of any one of Embodiments I-1 to I-7, I-9, I-11 to I-12, I-14 to I-15, I-17 to I-19, I-21, I-23 to I-24, I-26 to I-27, and I-29 to I-35, wherein A¹ is

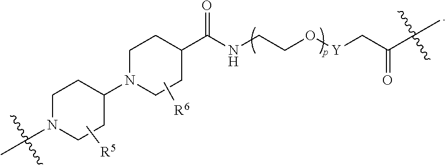

Embodiment I-45. The compound of any one of Embodiments I-1 to I-9, I-17 to I-21, and I-29 to I-44, wherein A² is absent.

Embodiment I-46. The compound of any one of Embodiments I-1 to I-7, I-10 to I-15, I-17 to I-19, I-22 to I-27 and I-29 to I-44, wherein A² is

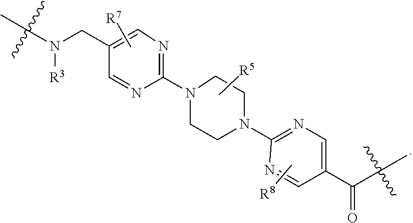

Embodiment I-47. The compound of any one of Embodiments I-1 to I-7, I-10 to I-15, I-17 to I-19, I-22 to I-27 and I-29 to I-44, wherein A² is

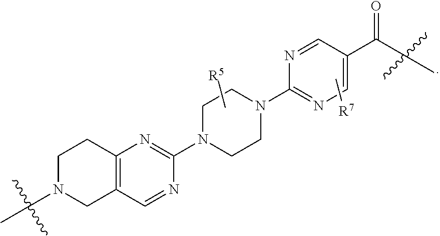

Embodiment I-48. The compound of any one of Embodiments I-1 to I-7, I-10 to I-15, I-17 to I-19, I-22 to I-27 and I-29 to I-44, wherein A² is

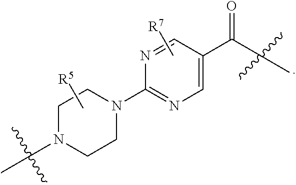

Embodiment I-49. The compound of any one of Embodiments I-1 to I-7, I-10 to I-15, I-17 to I-19, I-22 to I-27 and I-29 to I-44, wherein A² is

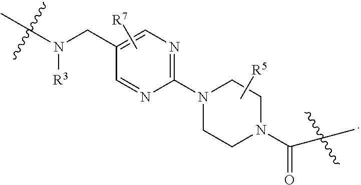

Embodiment I-50. The compound of any one of Embodiments I-1 to I-7, I-10 to I-15, I-17 to I-19, I-22 to I-27 and I-29 to I-44, wherein A² is

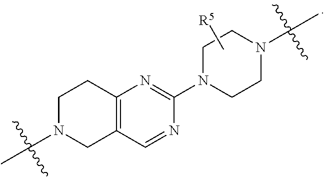

Embodiment I-51. The compound of any one of Embodiments I-1 to I-7, I-10 to I-15, I-17 to I-19, I-22 to I-27 and I-29 to I-44, wherein A² is

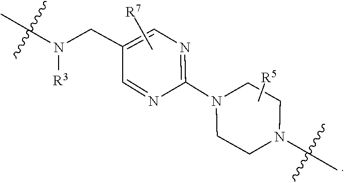

Embodiment I-52. The compound of any one of Embodiments I-1 to I-7, I-10 to I-15, I-17 to I-19, I-22 to I-27 and I-29 to I-44, wherein A² is

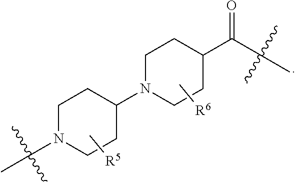

Embodiment I-53. The compound of any one of Embodiments I-1 to I-7, I-10 to I-15, I-17 to I-19, I-22 to I-27 and I-29 to I-44, wherein A² is

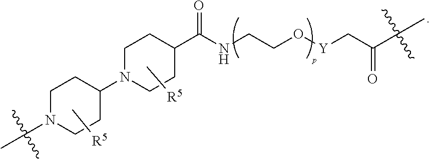

Embodiment I-54. The compound of any one of Embodiments I-1 to I-53, wherein B is

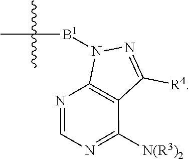

Embodiment I-55. The compound of any one of Embodiments I-1 to I-53, wherein B is

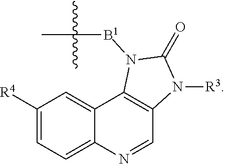

Embodiment I-56. The compound of any one of Embodiments I-1 to I-53, wherein B¹ is

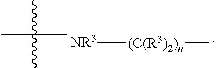

Embodiment I-57. The compound of any one of Embodiments I-1 to I-53, wherein B¹ is

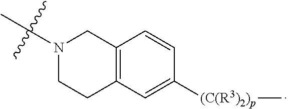

Embodiment I-58. The compound of any one of Embodiments I-1 to I-57, wherein R⁴ is 5-12 membered heteroaryl, optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl.

Embodiment I-59. The compound of any one of Embodiments I-1 to I-58, or a pharmaceutically acceptable salt or tautomer thereof, wherein compound has the following formula:

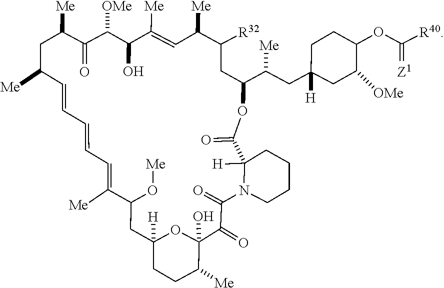

Embodiment I-60. A compound selected from the group consisting of.

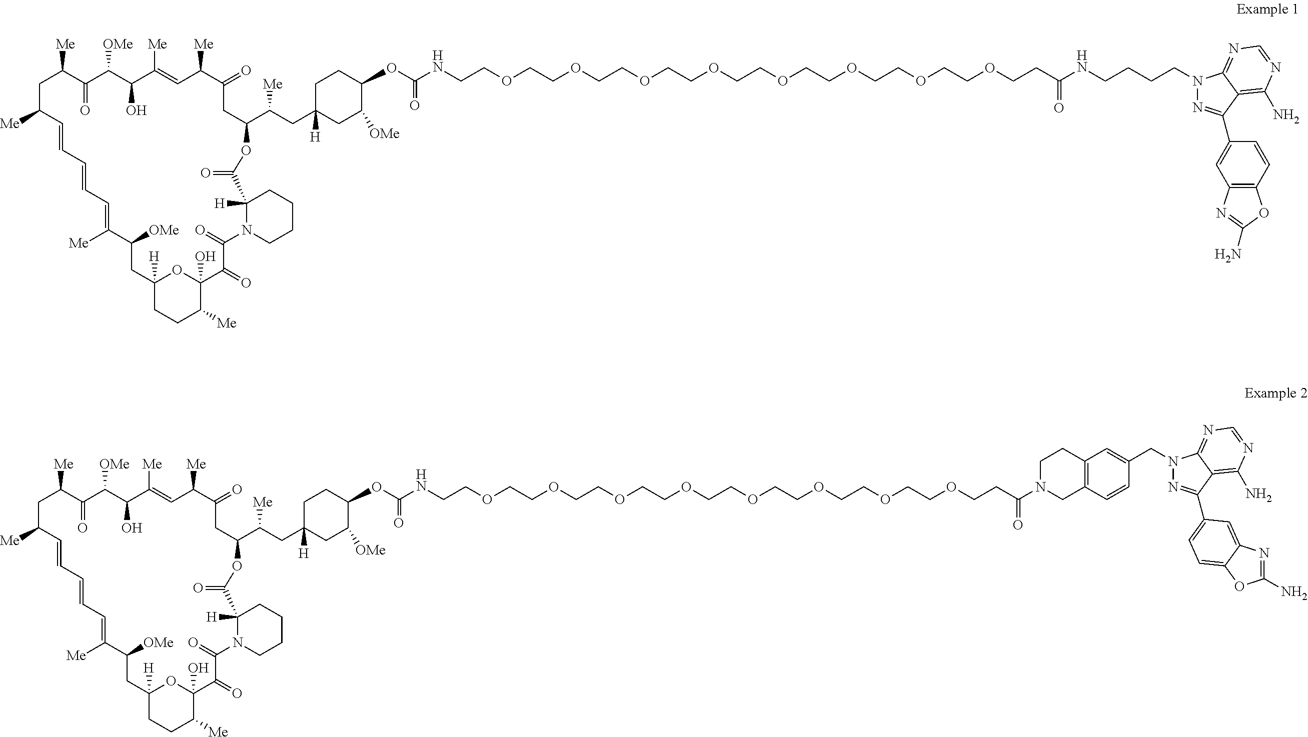

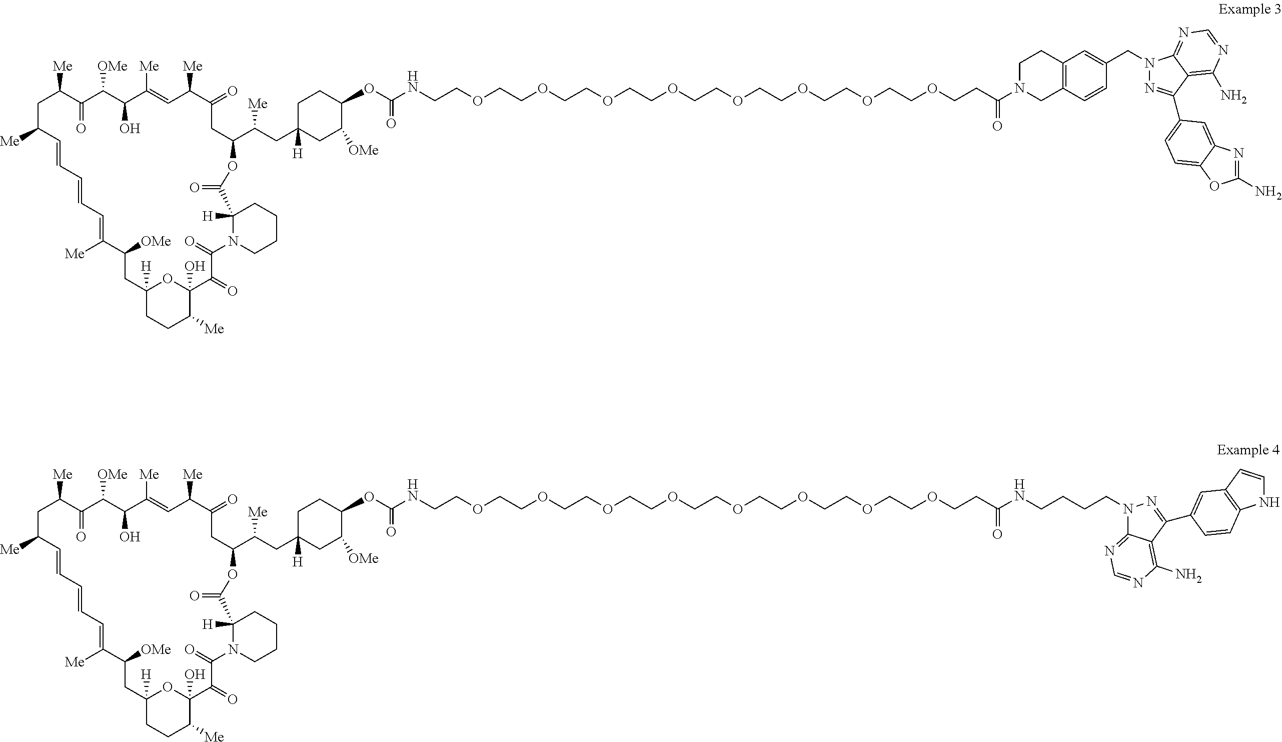

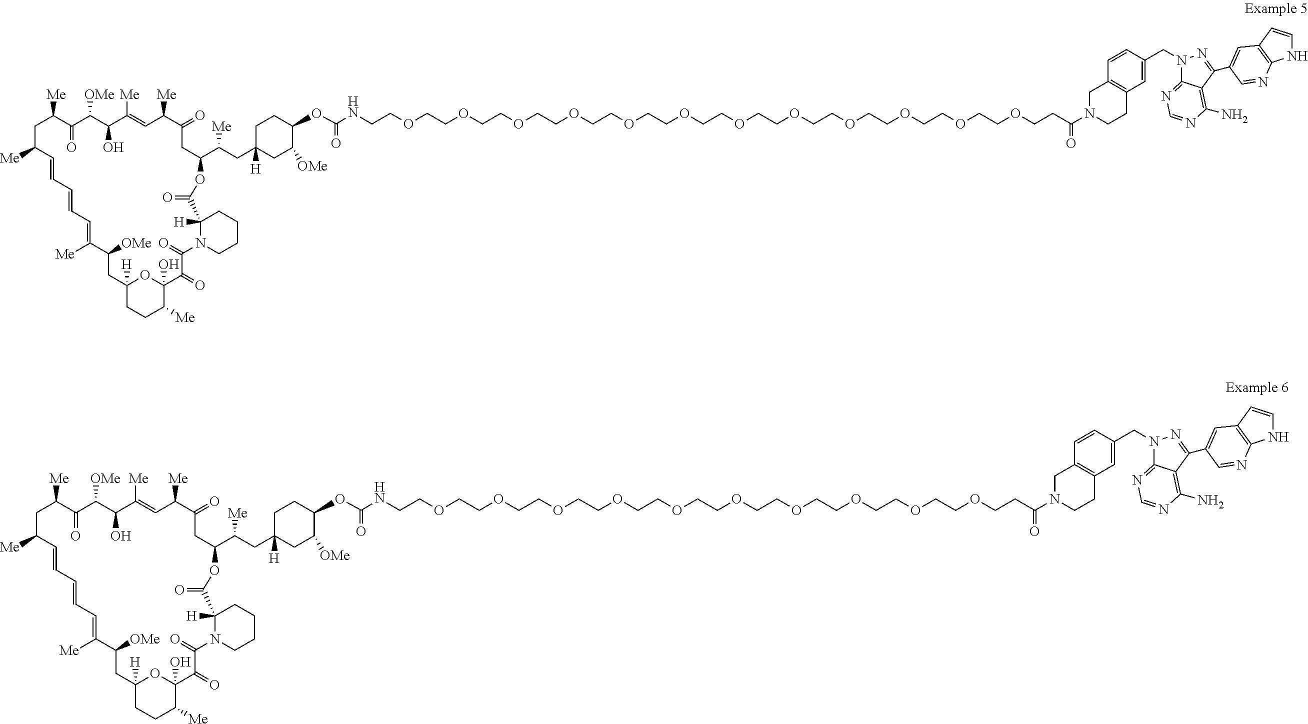

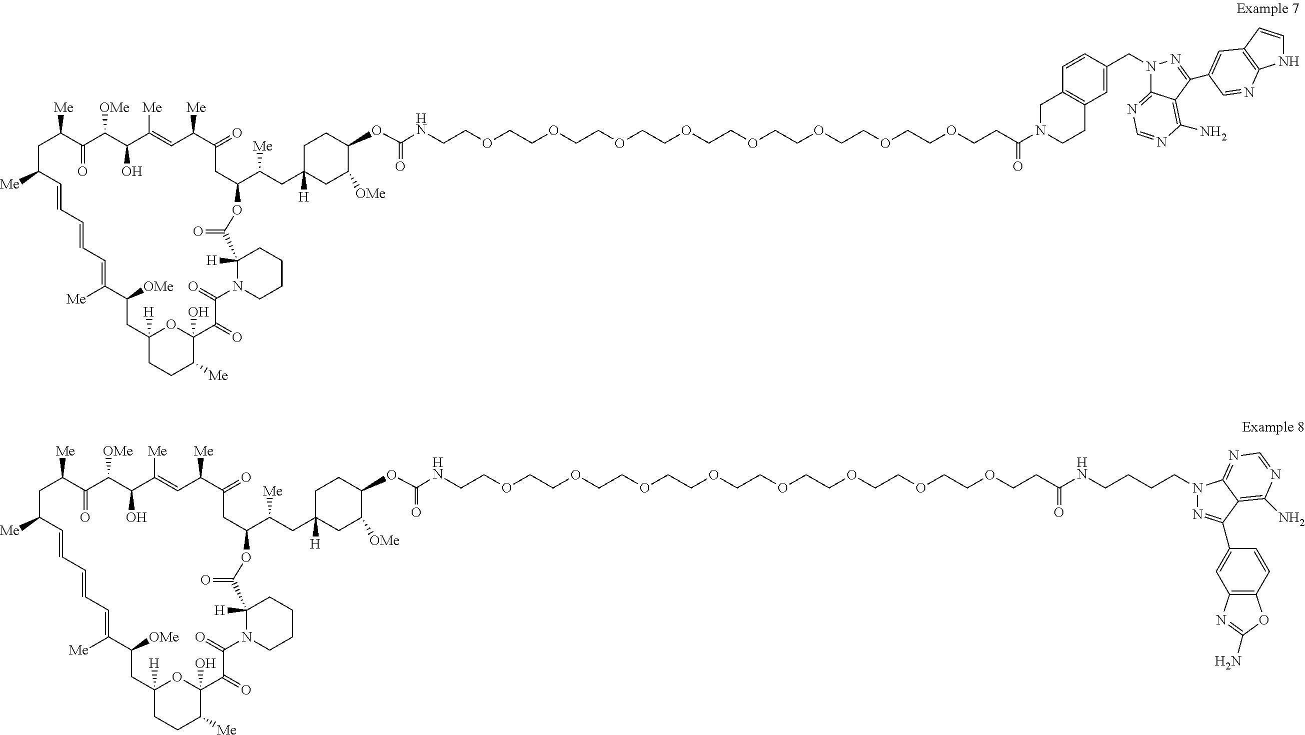

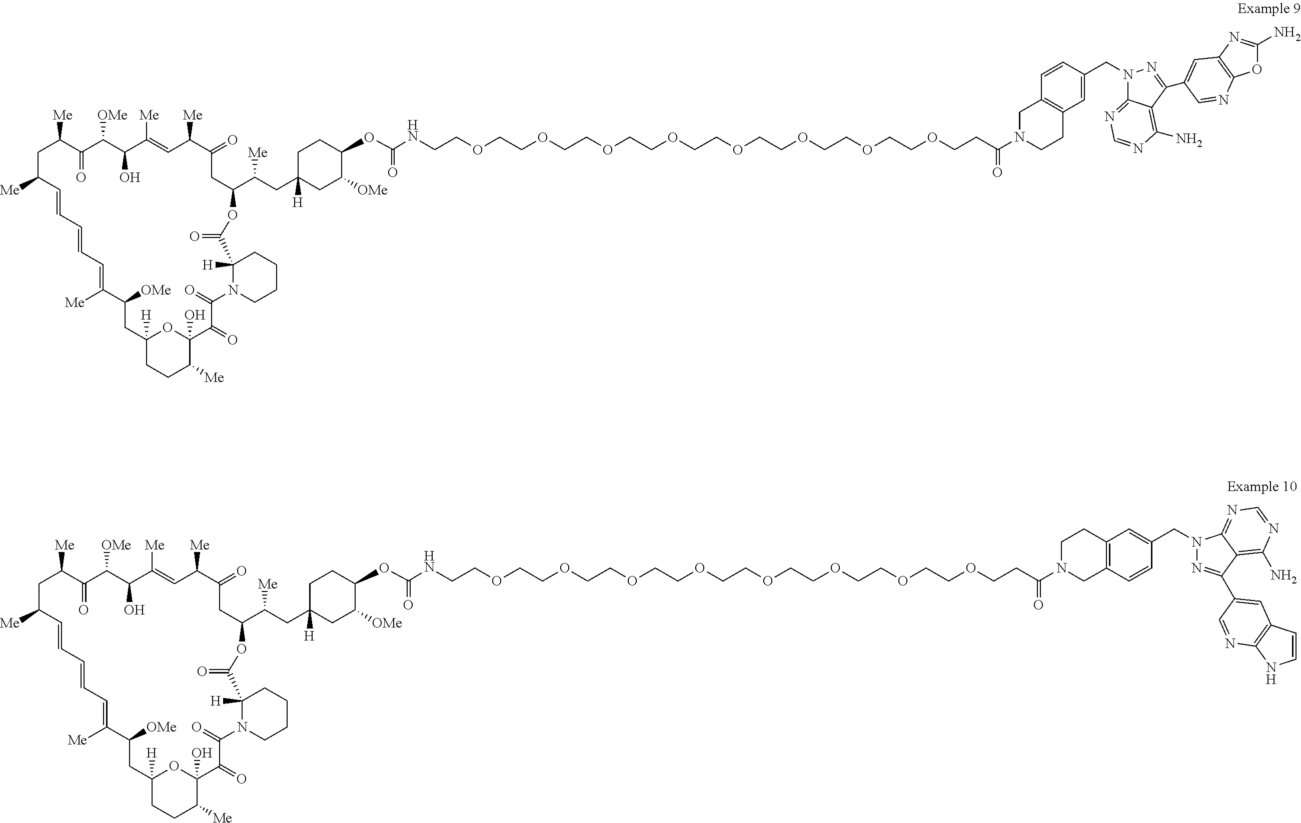

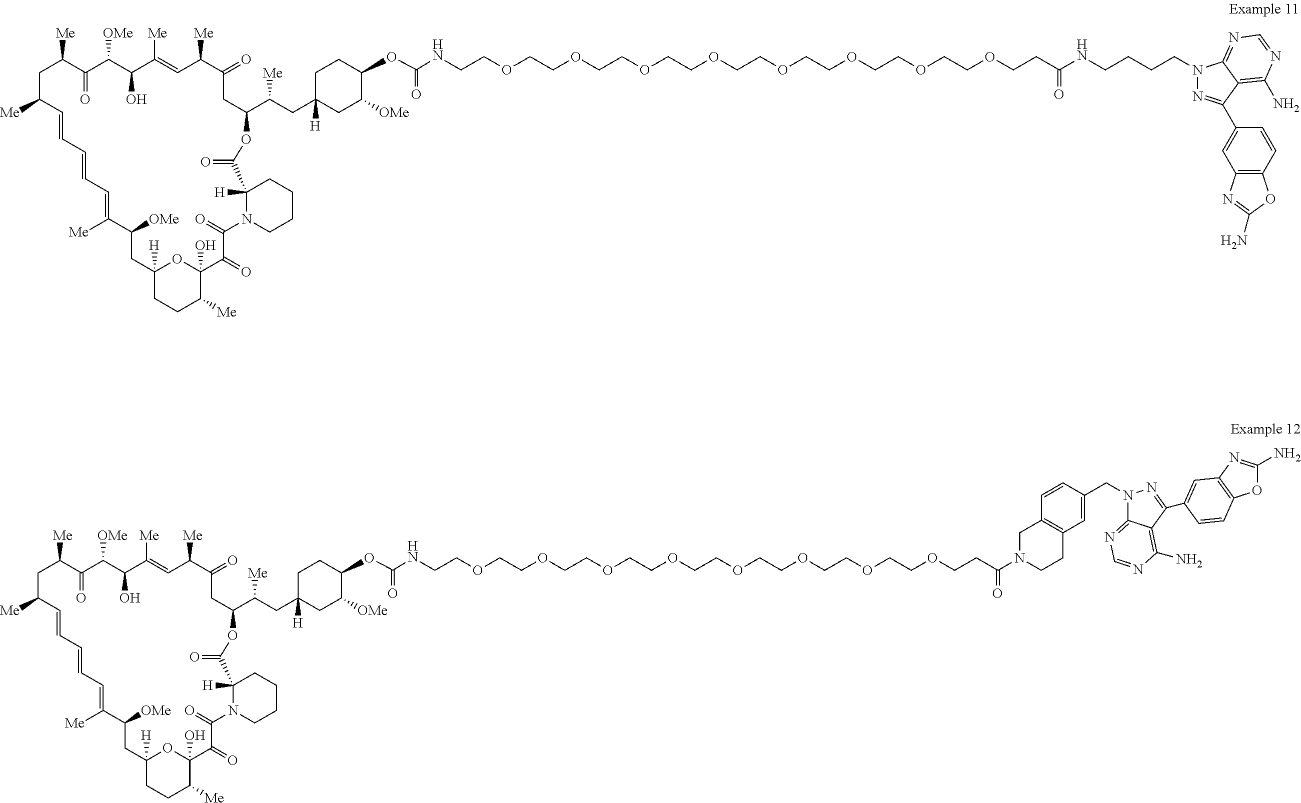

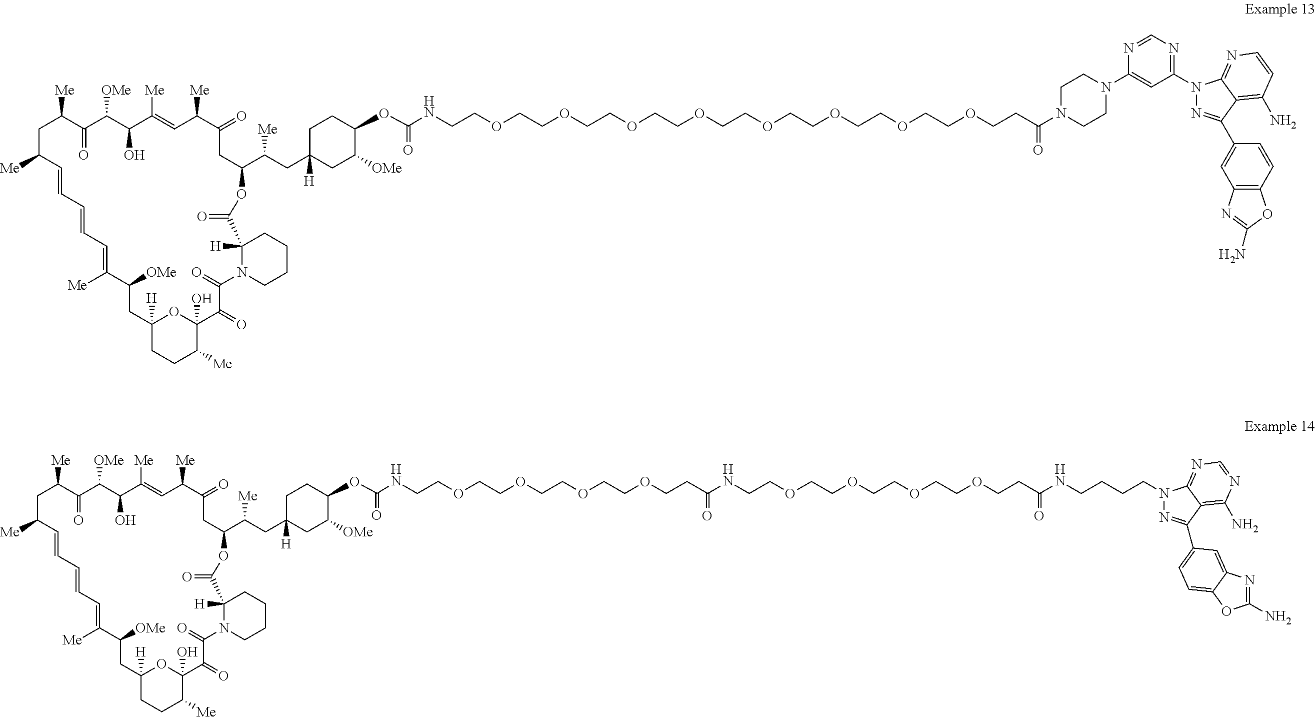

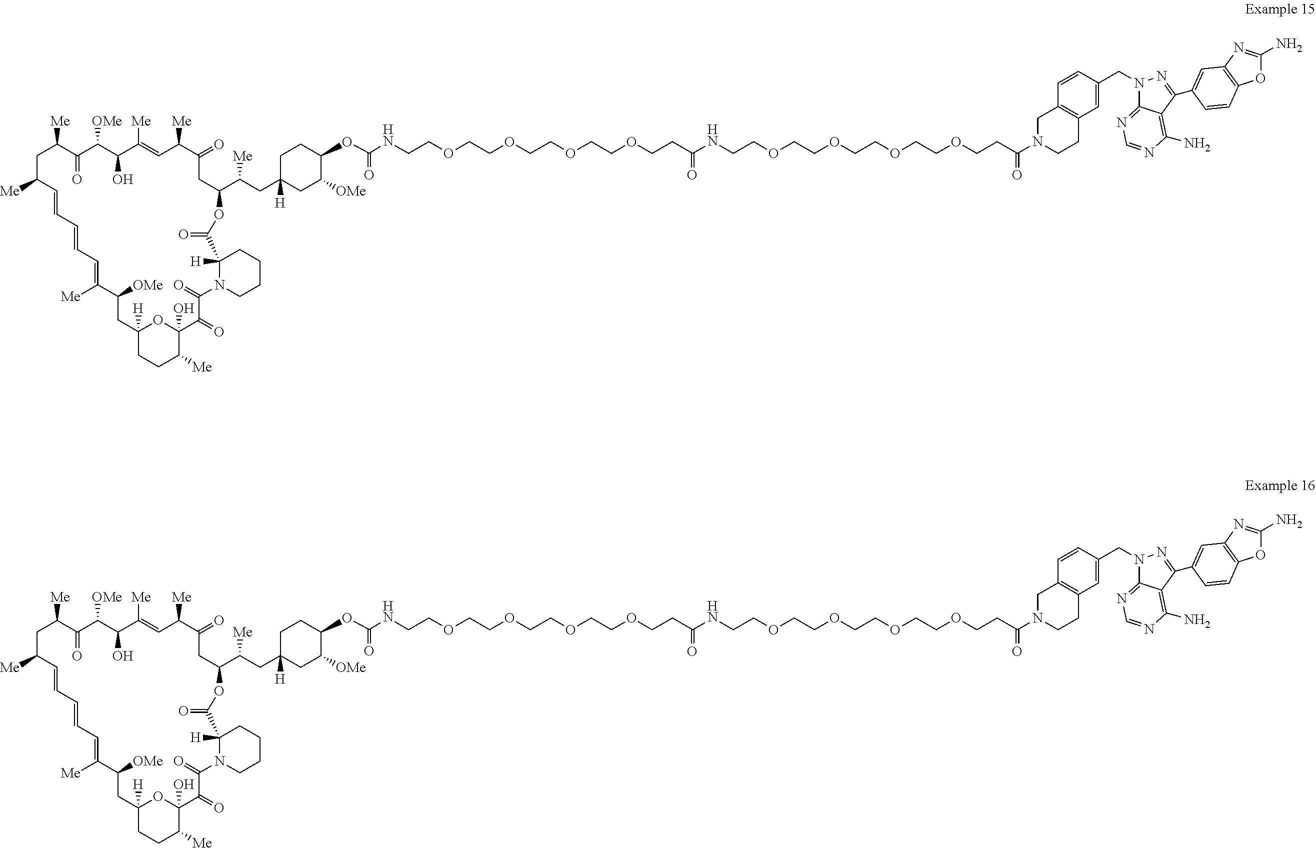

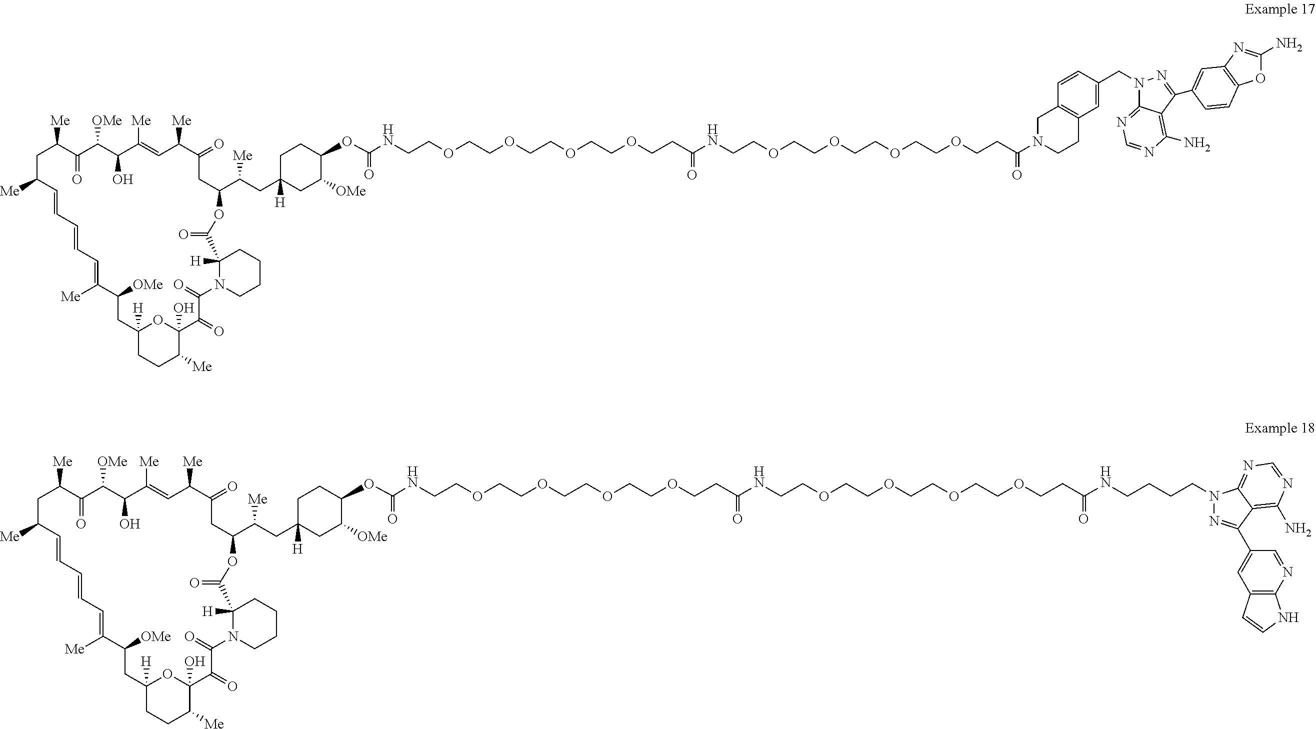

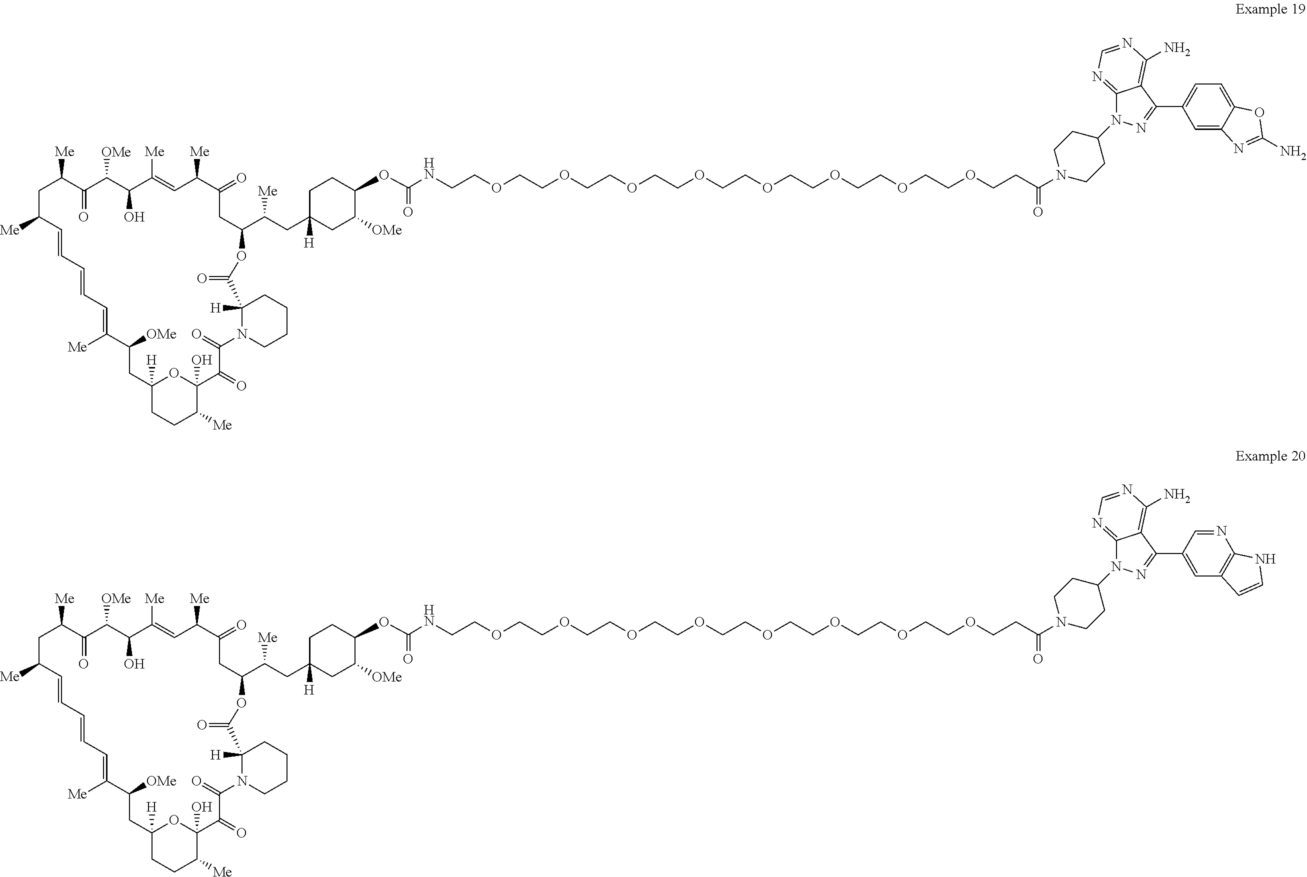

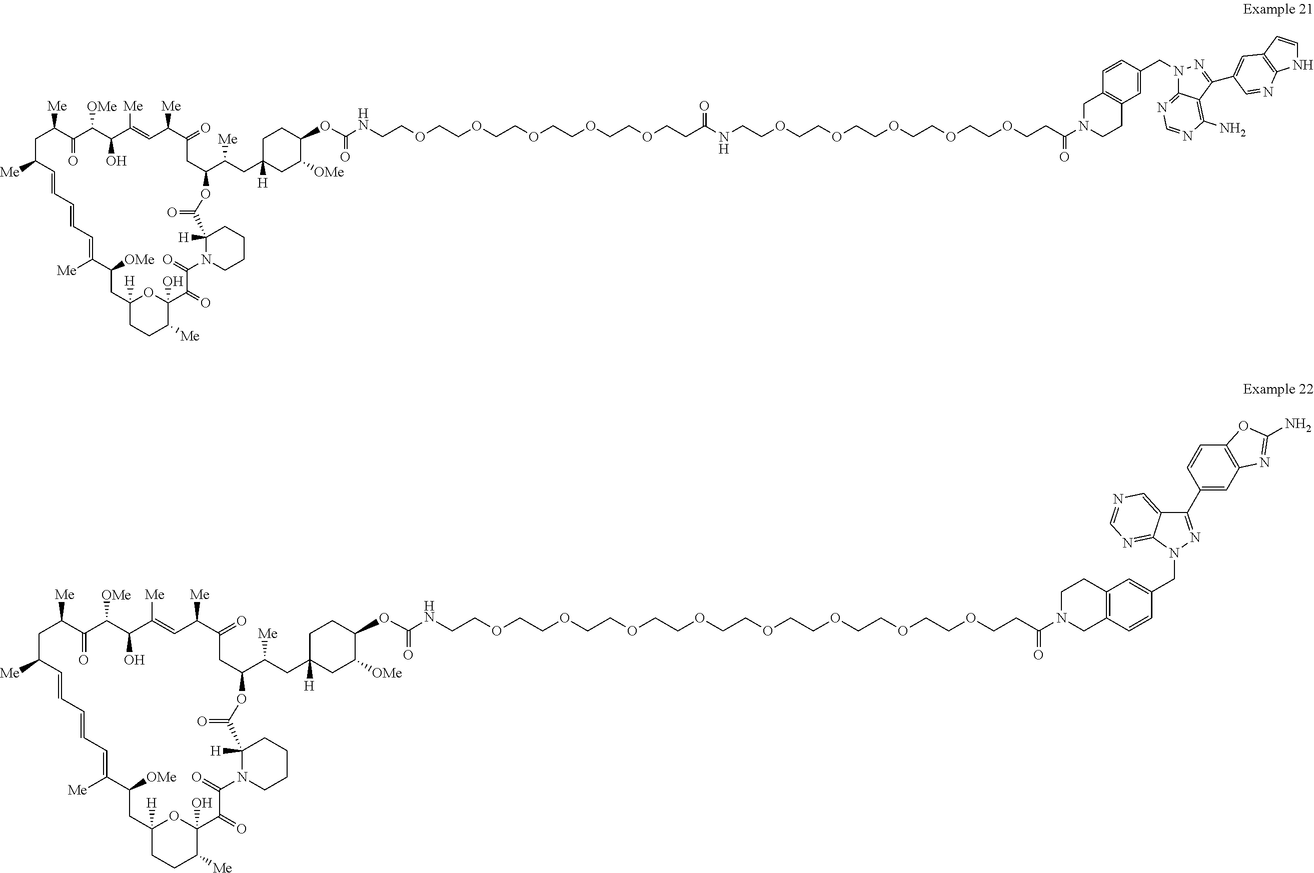

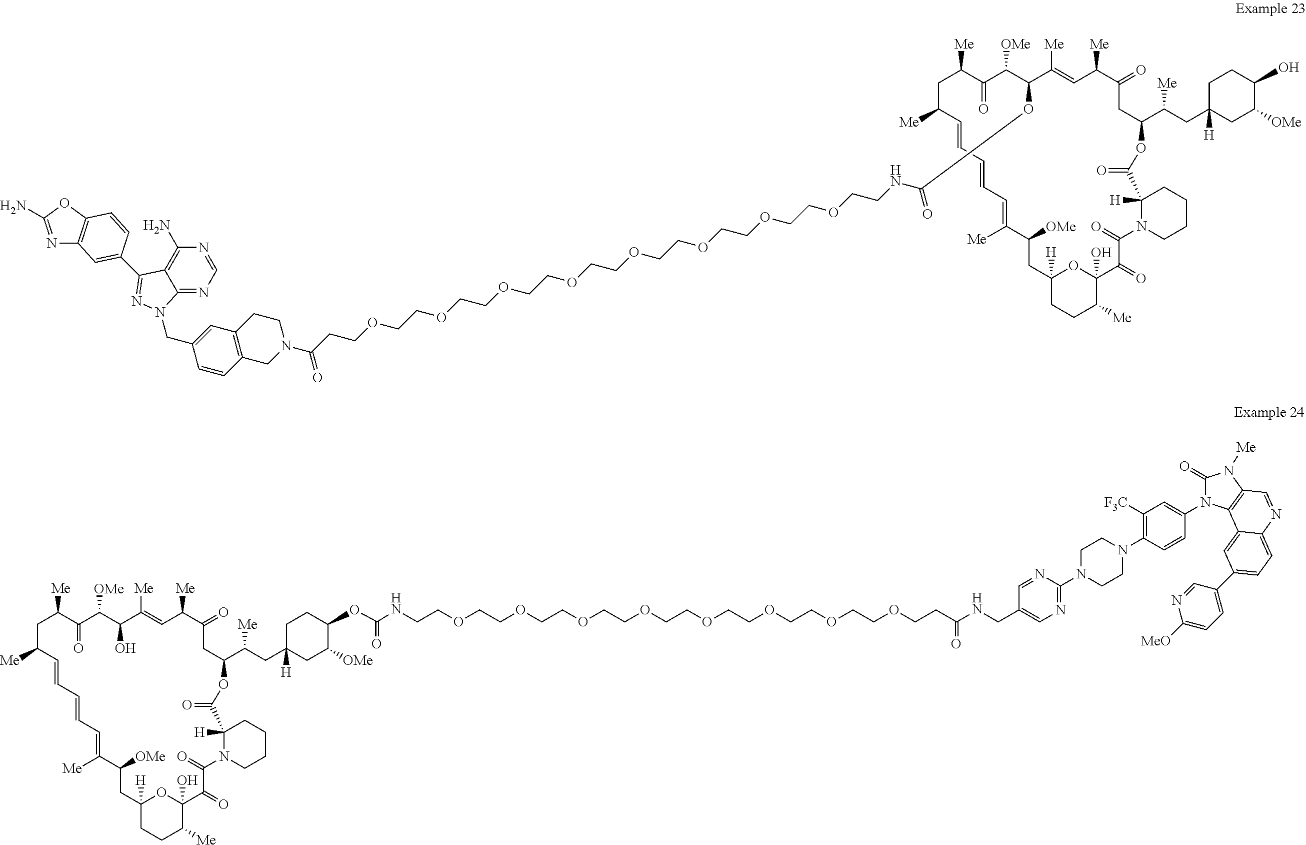

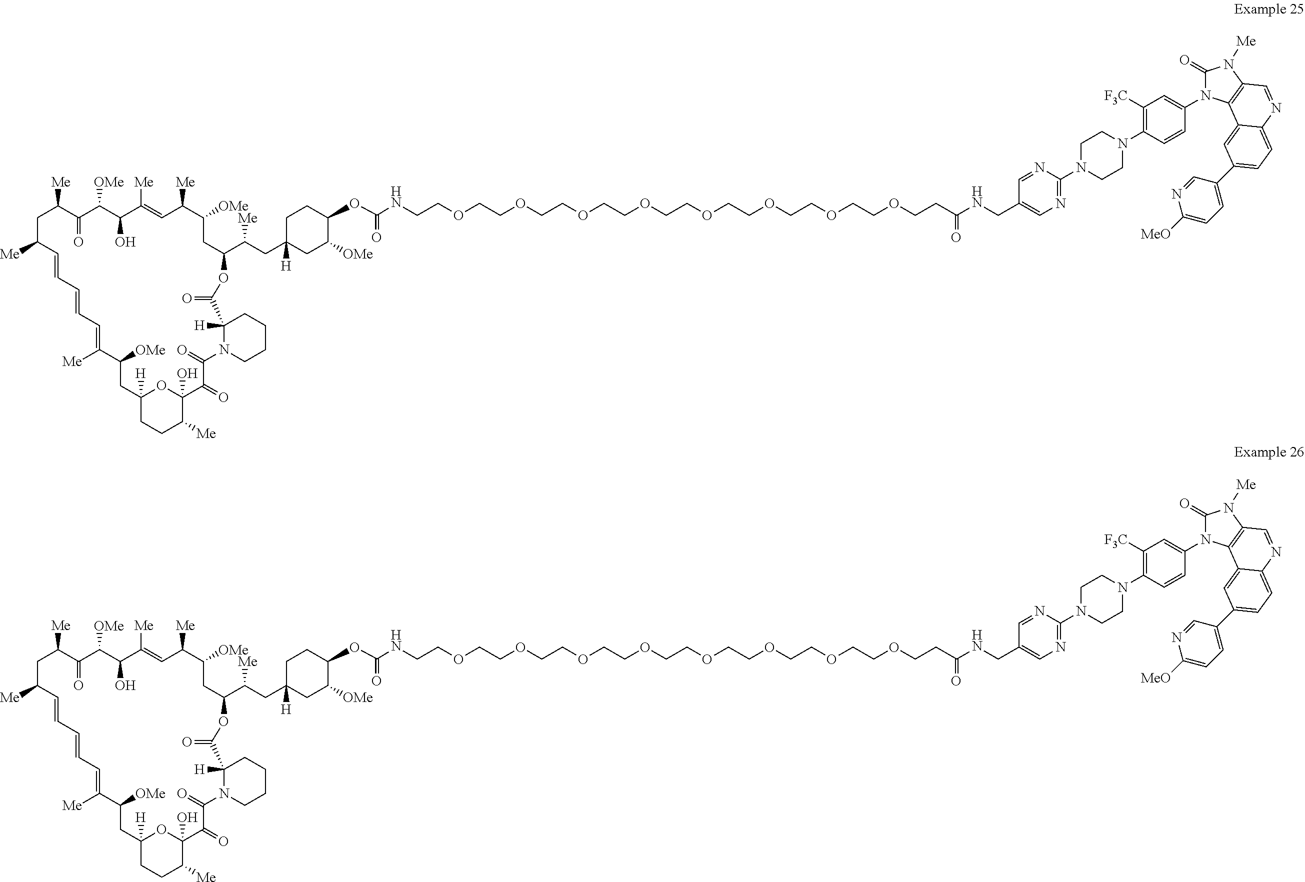

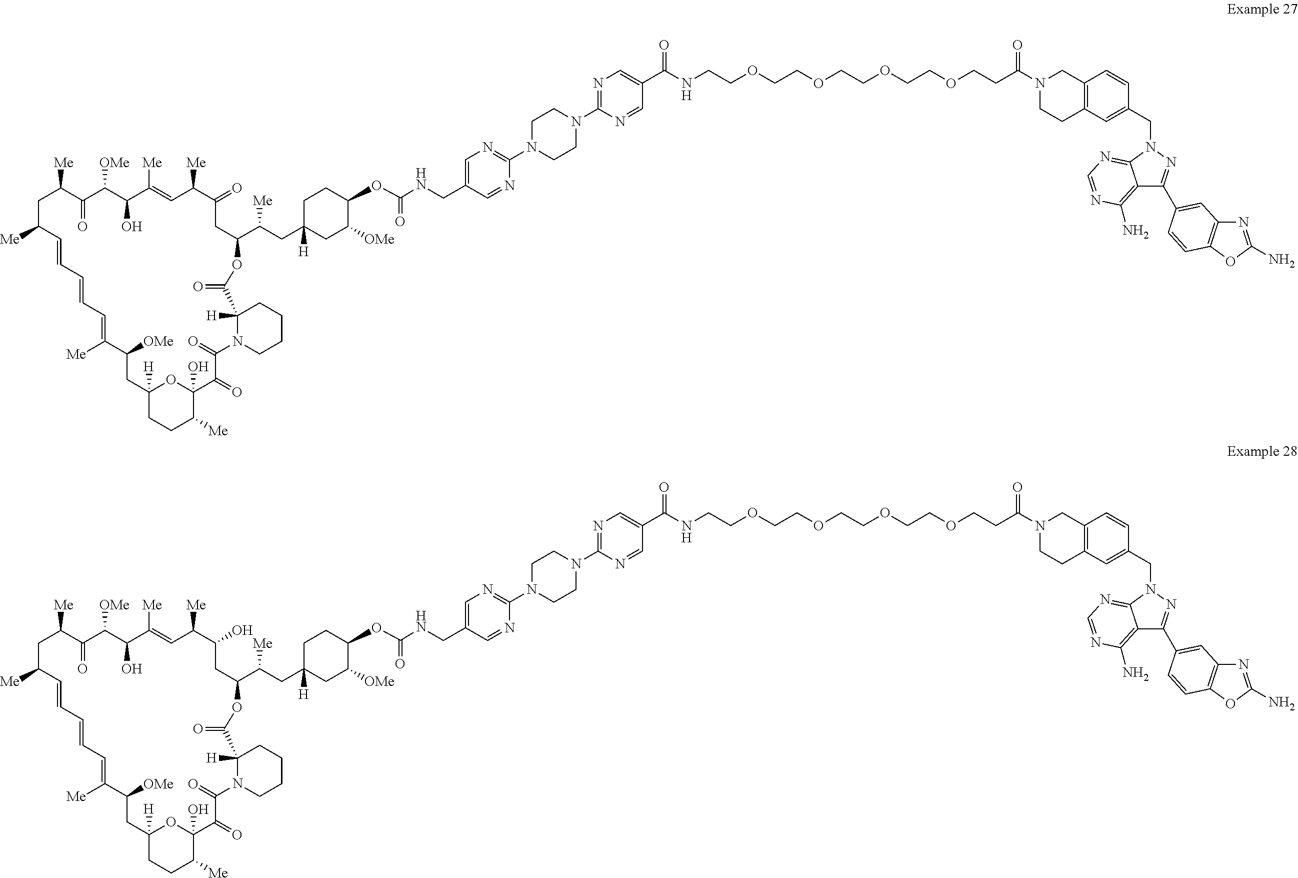

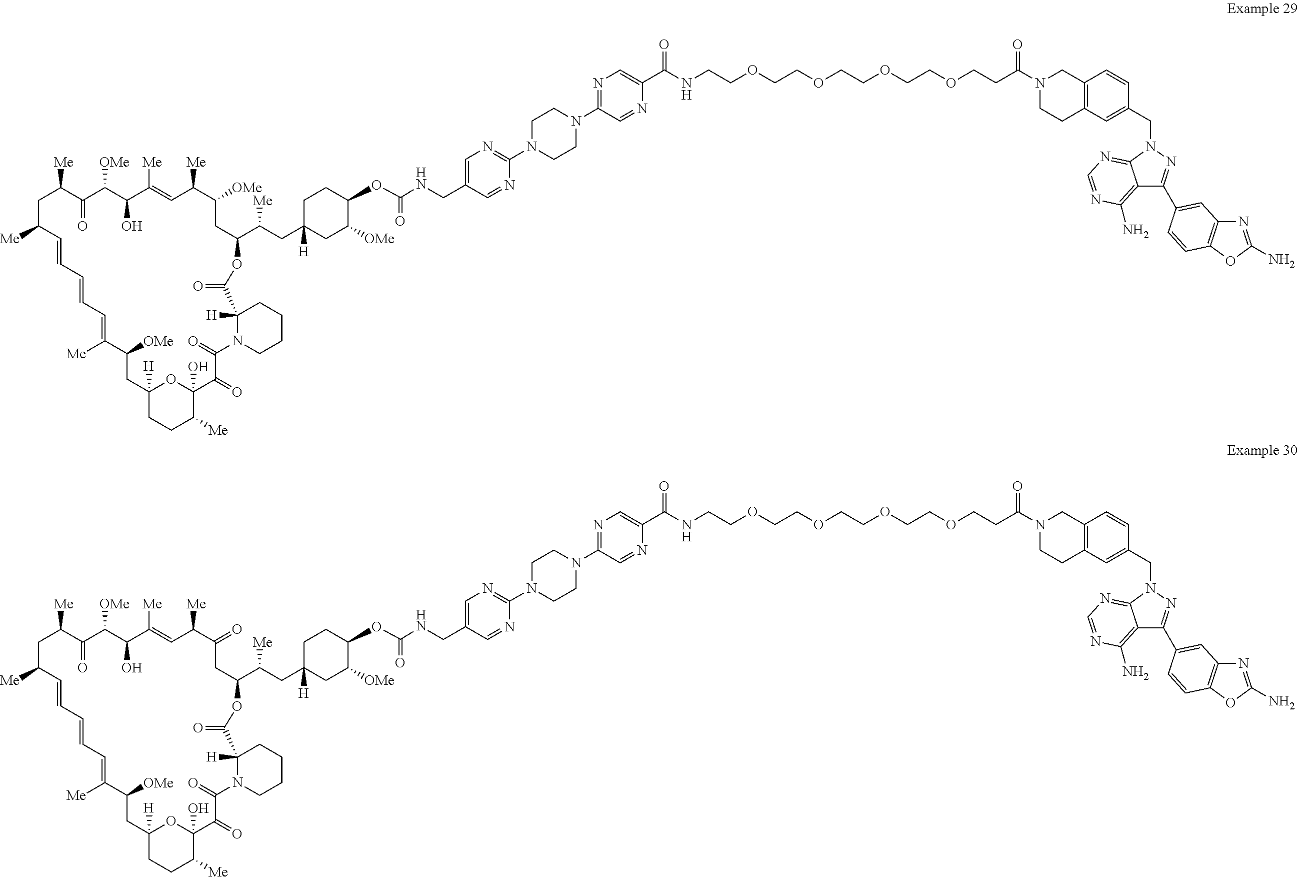

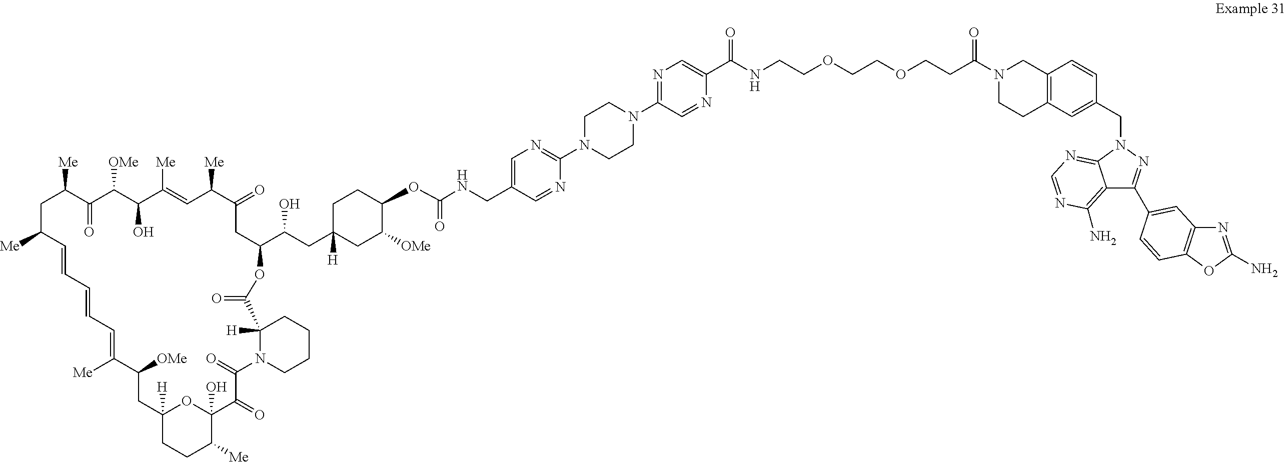 [SMILES: CO[C@H]1C[C@@H]2CC[C@@H](C)[C@@](O)(O2)C(=O)C(=O)N2CCCC[C@H]2C(=O)O[C@H]([C@H](O)C[C@@H]2CC[C@@H](OC(=O)NCc3cnc(N4CCN(c5cnc(C(=O)NCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

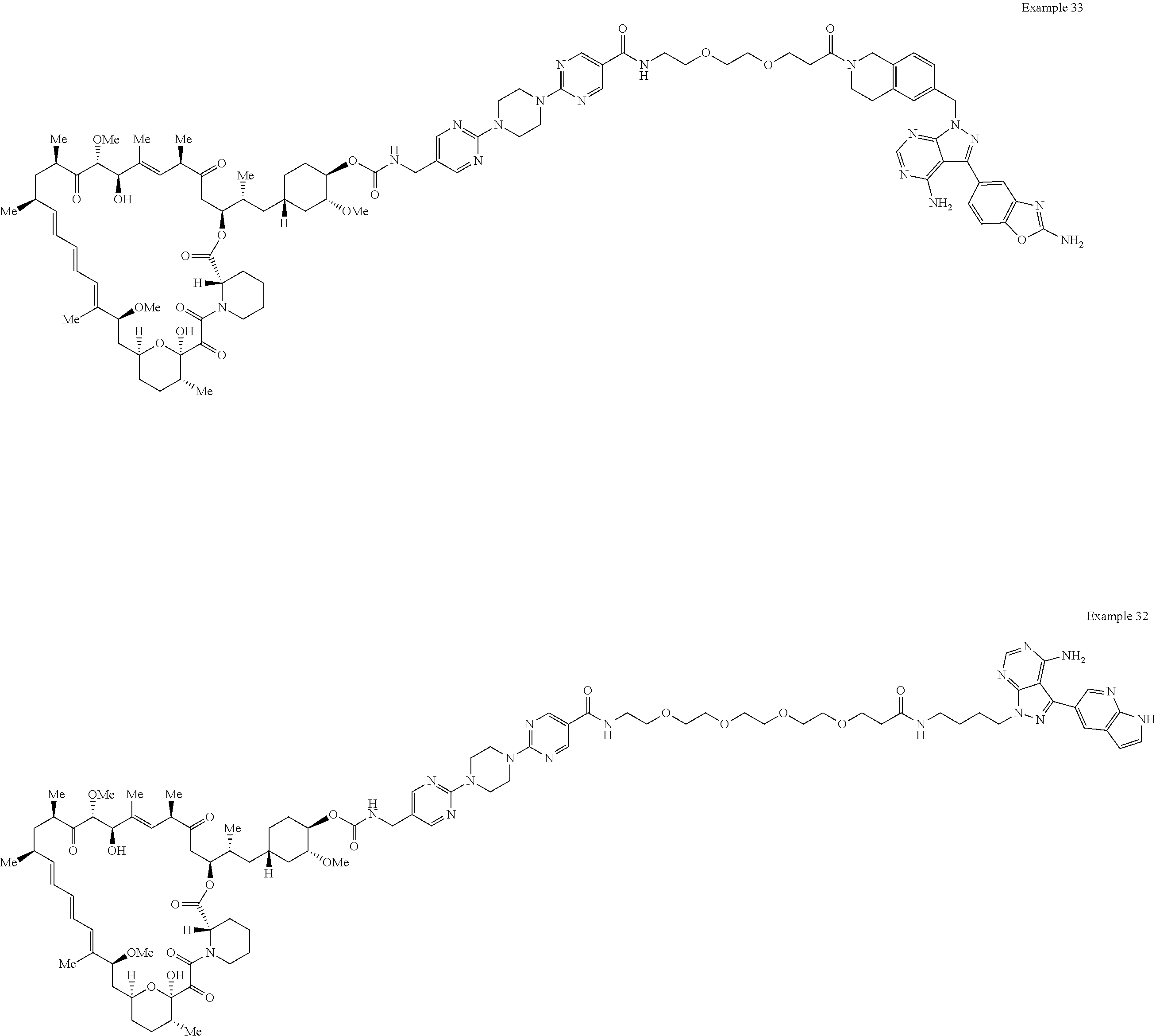

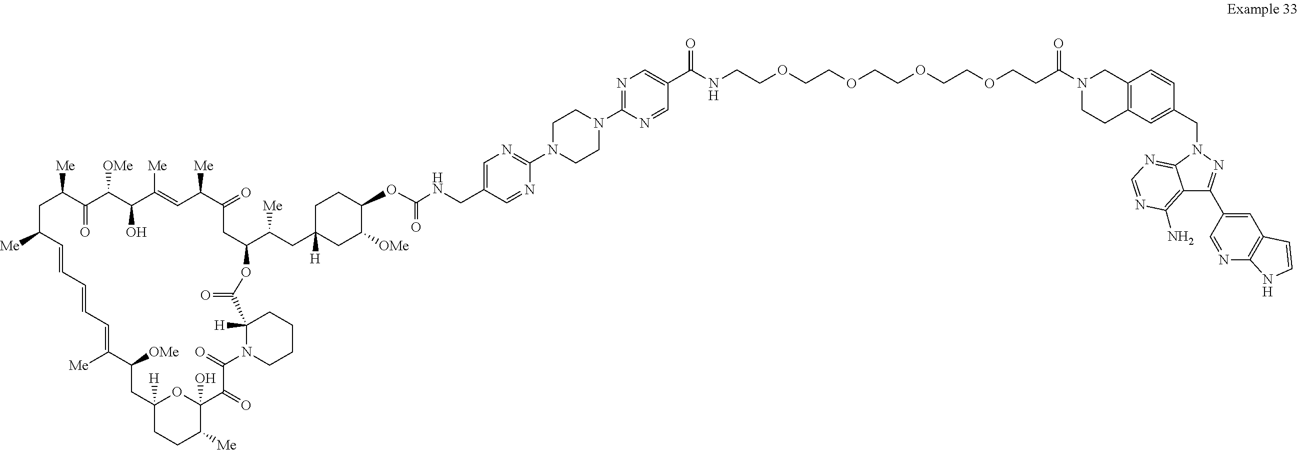 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

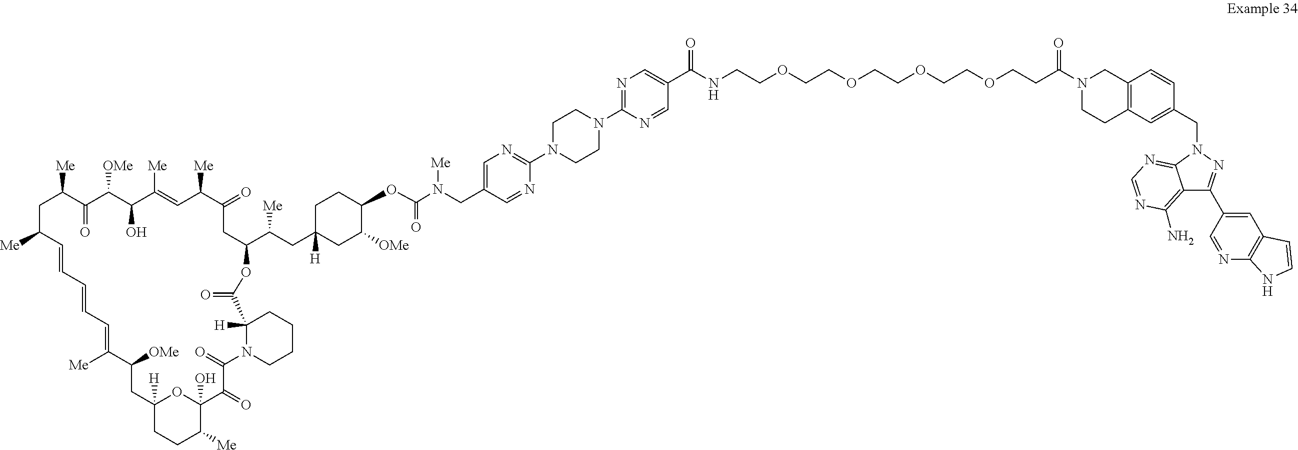 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)Cc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

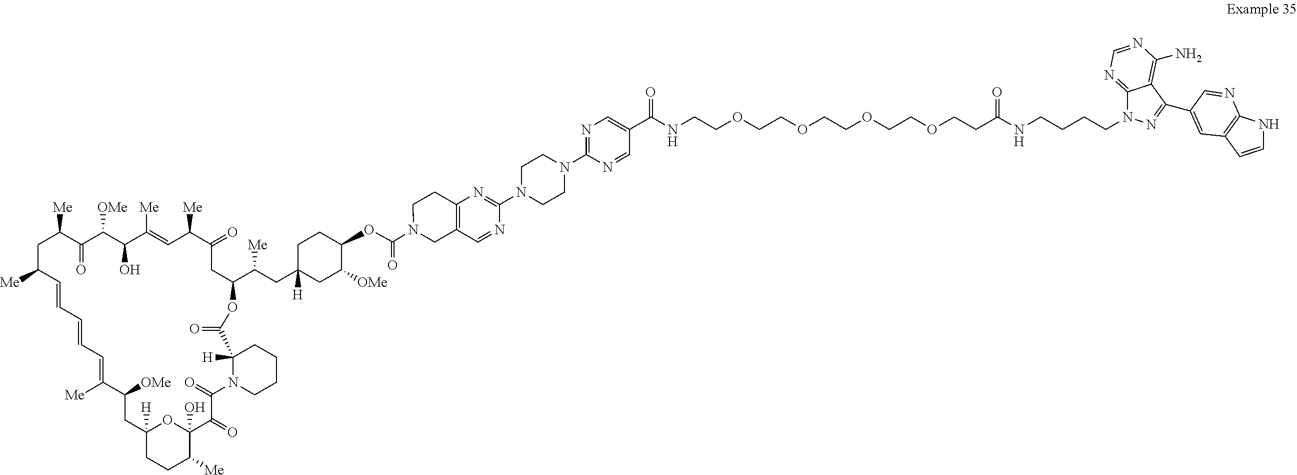 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)NCCCCn7nc(-c8cnc9[nH]ccc9c8)c8c(N)ncnc87)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

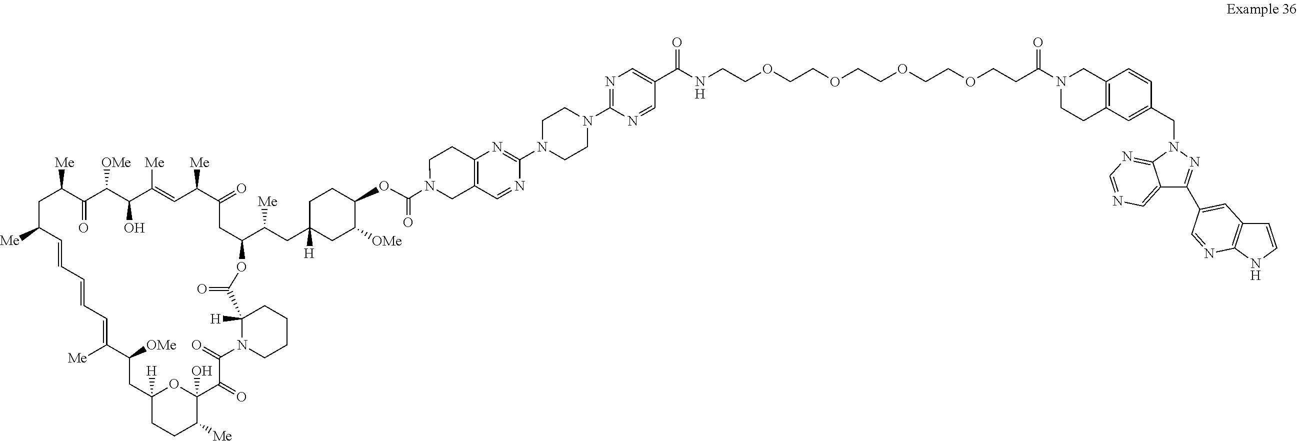 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10cncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

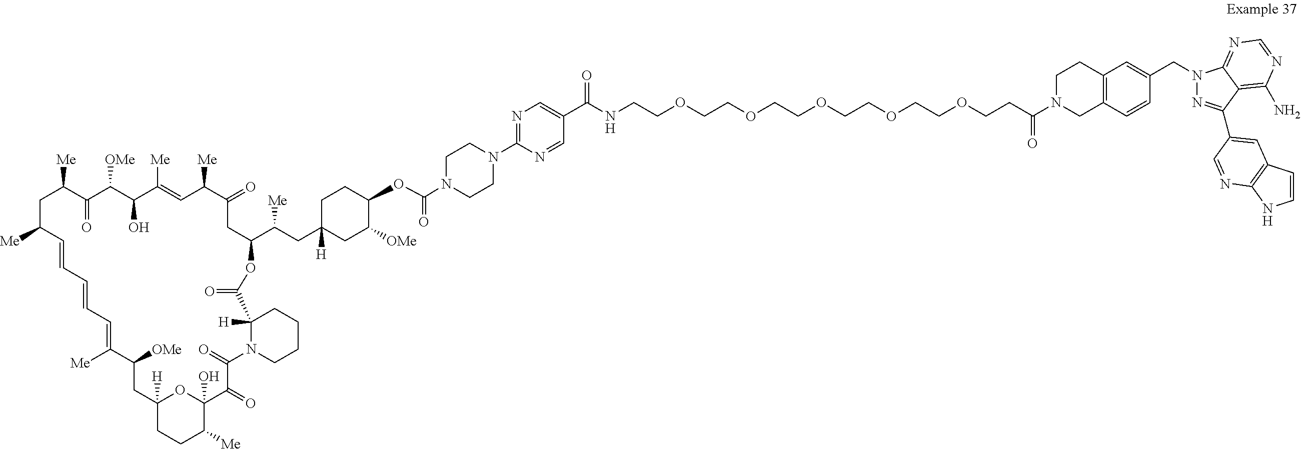 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(c4ncc(C(=O)NCCOCCOCCOCCOCCOCCC(=O)N5CCc6cc(Cn7nc(-c8cnc9[nH]ccc9c8)c8c(N)ncnc87)ccc6C5)cn4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C]

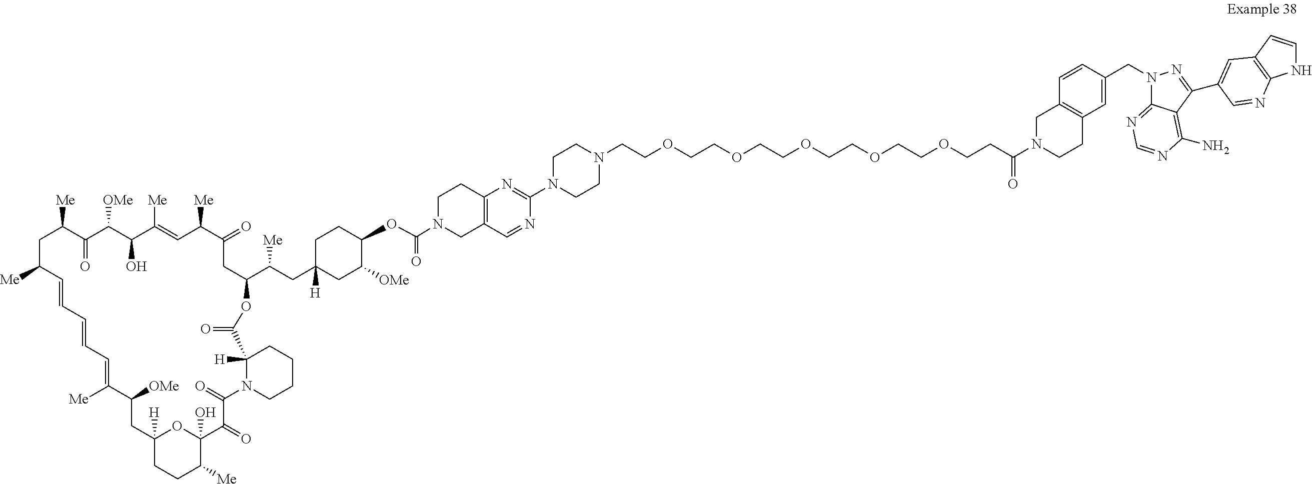 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCOCCOCCOCCOCCC(=O)N6CCc7cc(Cn8nc(-c9cnc%10[nH]ccc%10c9)c9c(N)ncnc98)ccc7C6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

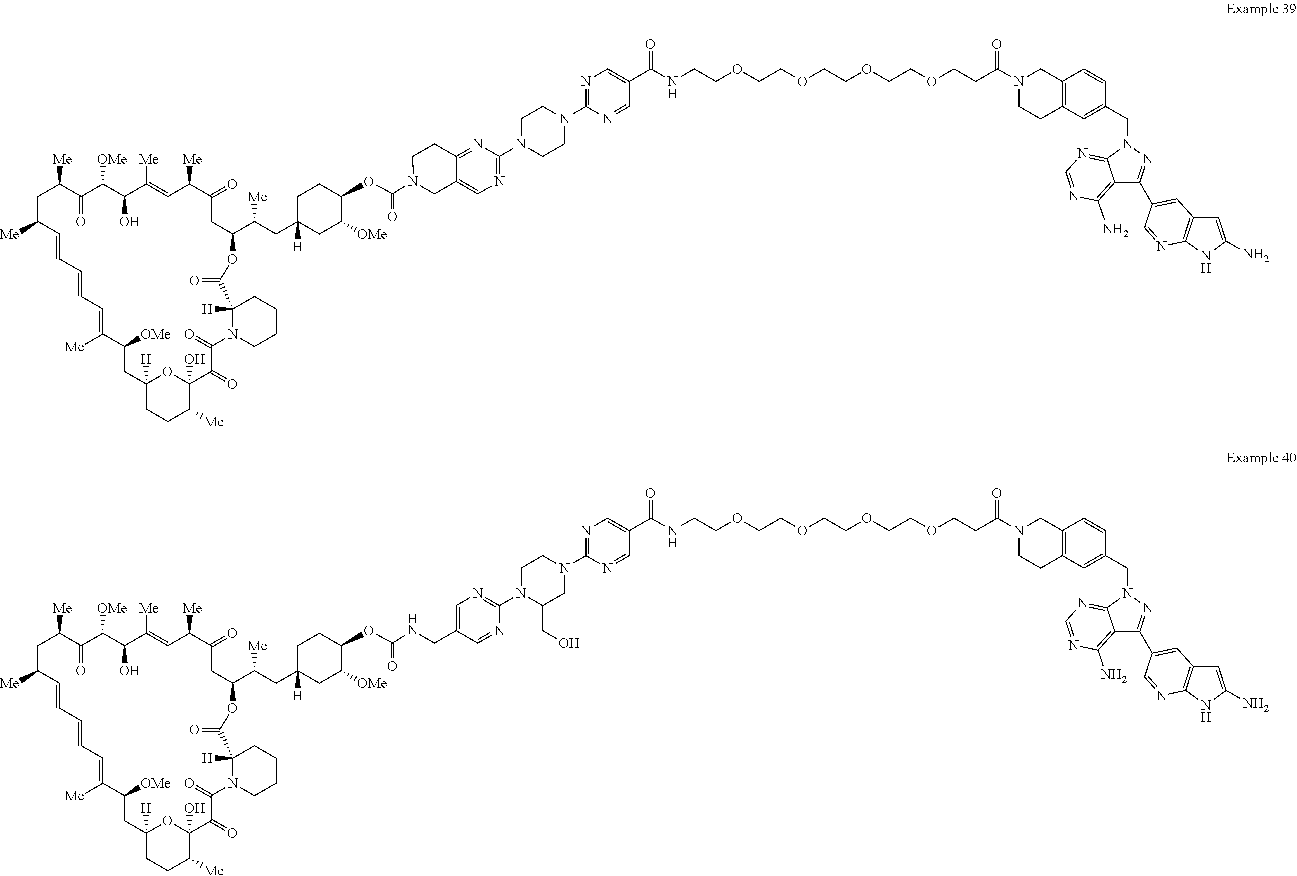

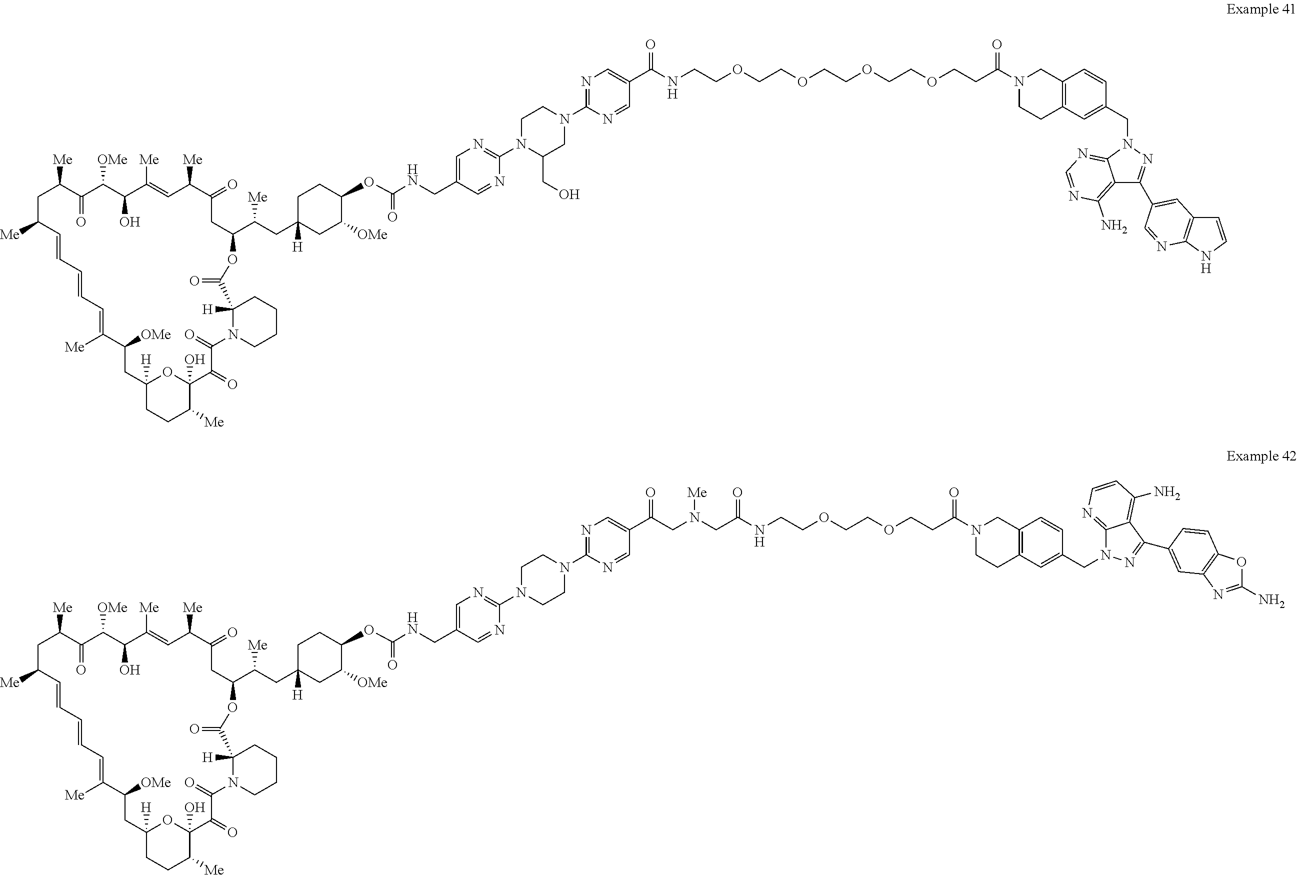

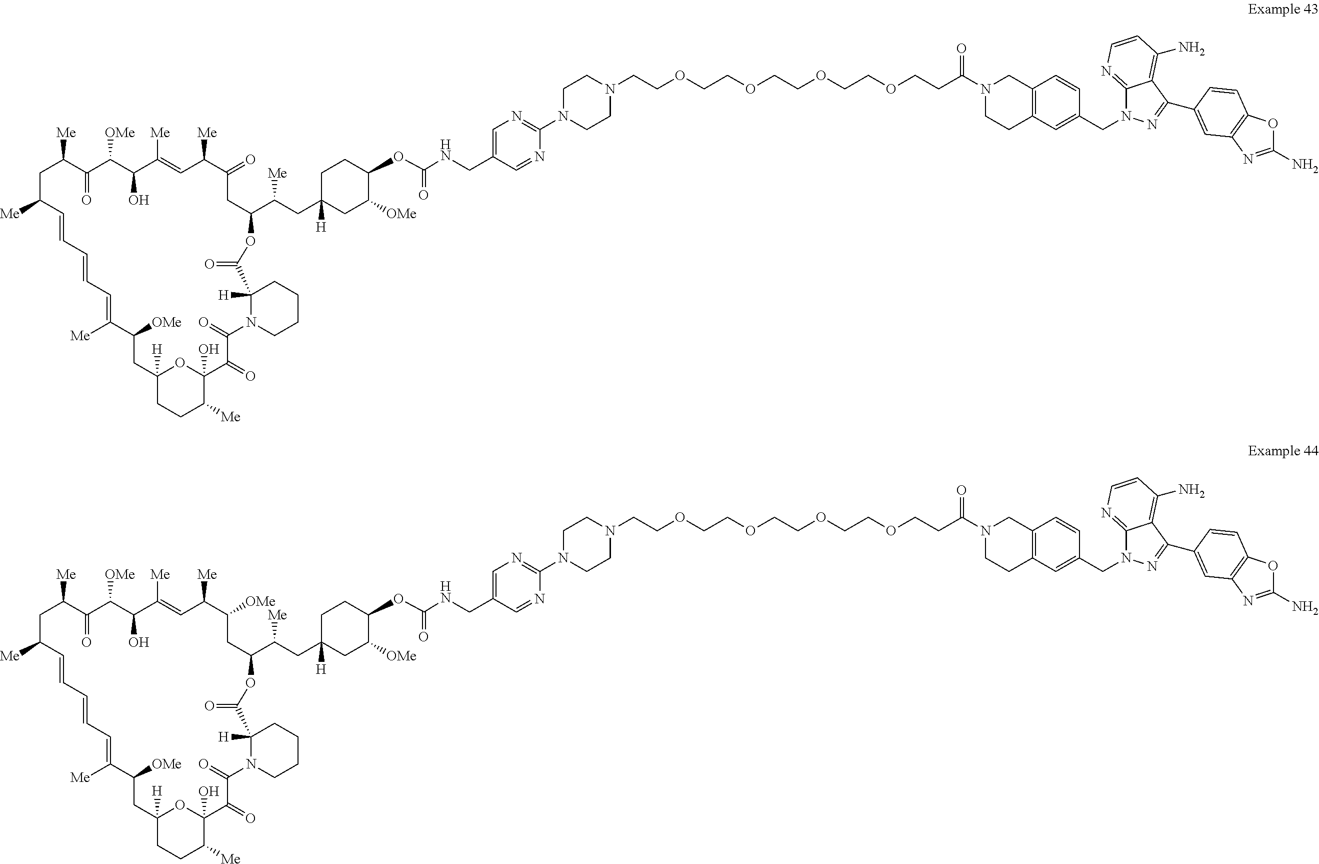

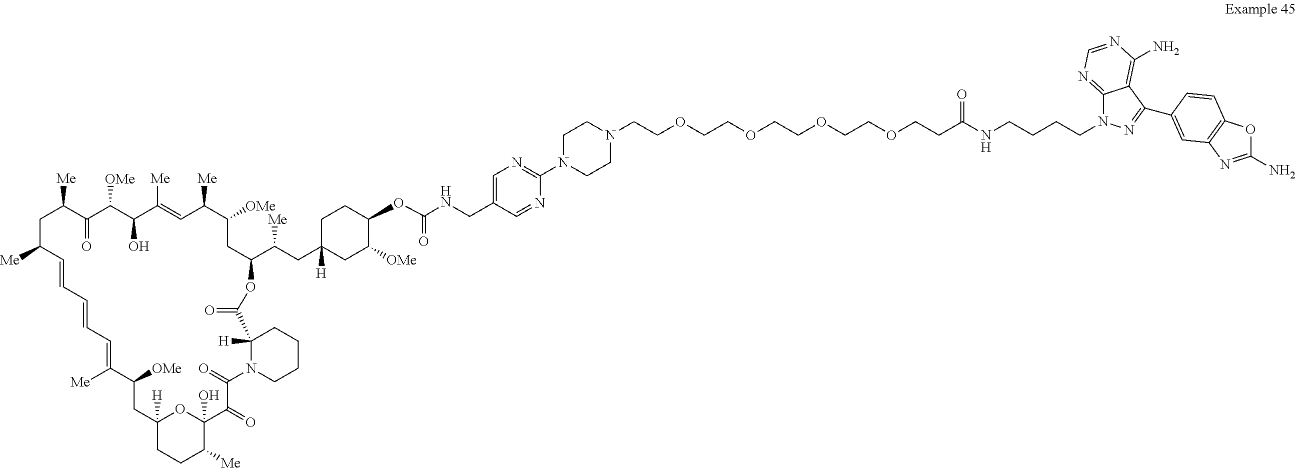 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)NCCCCn5nc(-c6ccc7oc(N)nc7c6)c6c(N)ncnc65)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

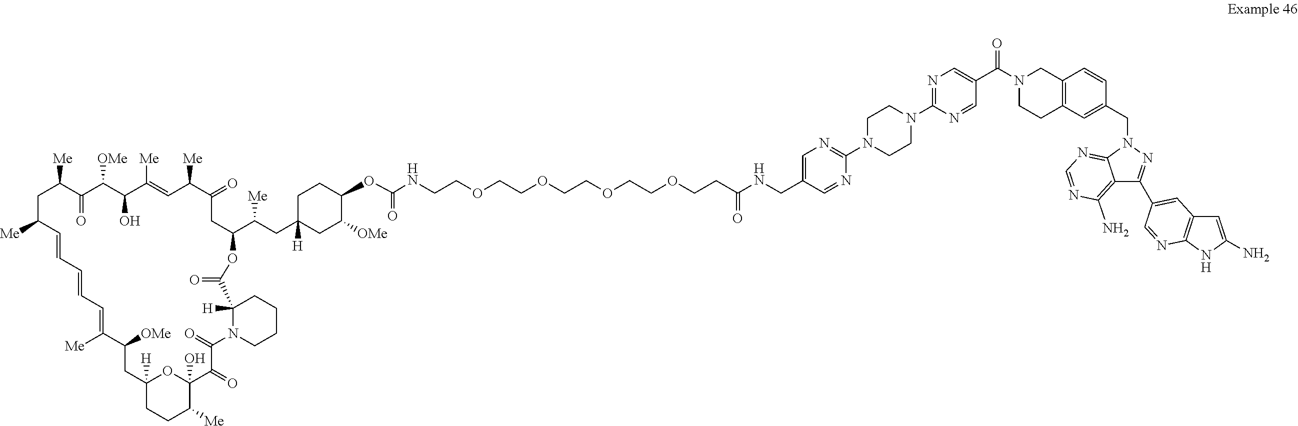 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9cnc%10[nH]c(N)cc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

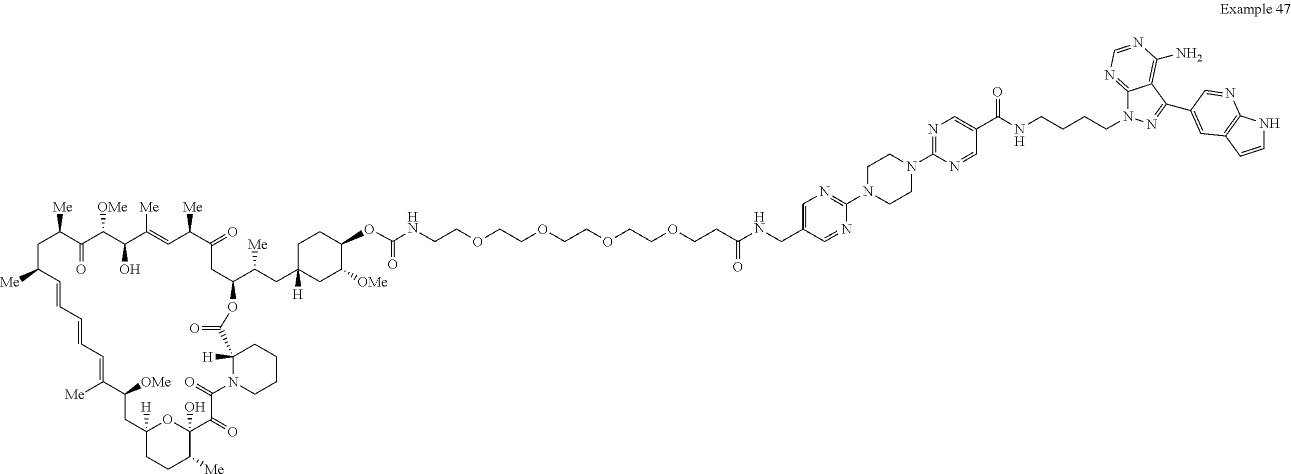 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

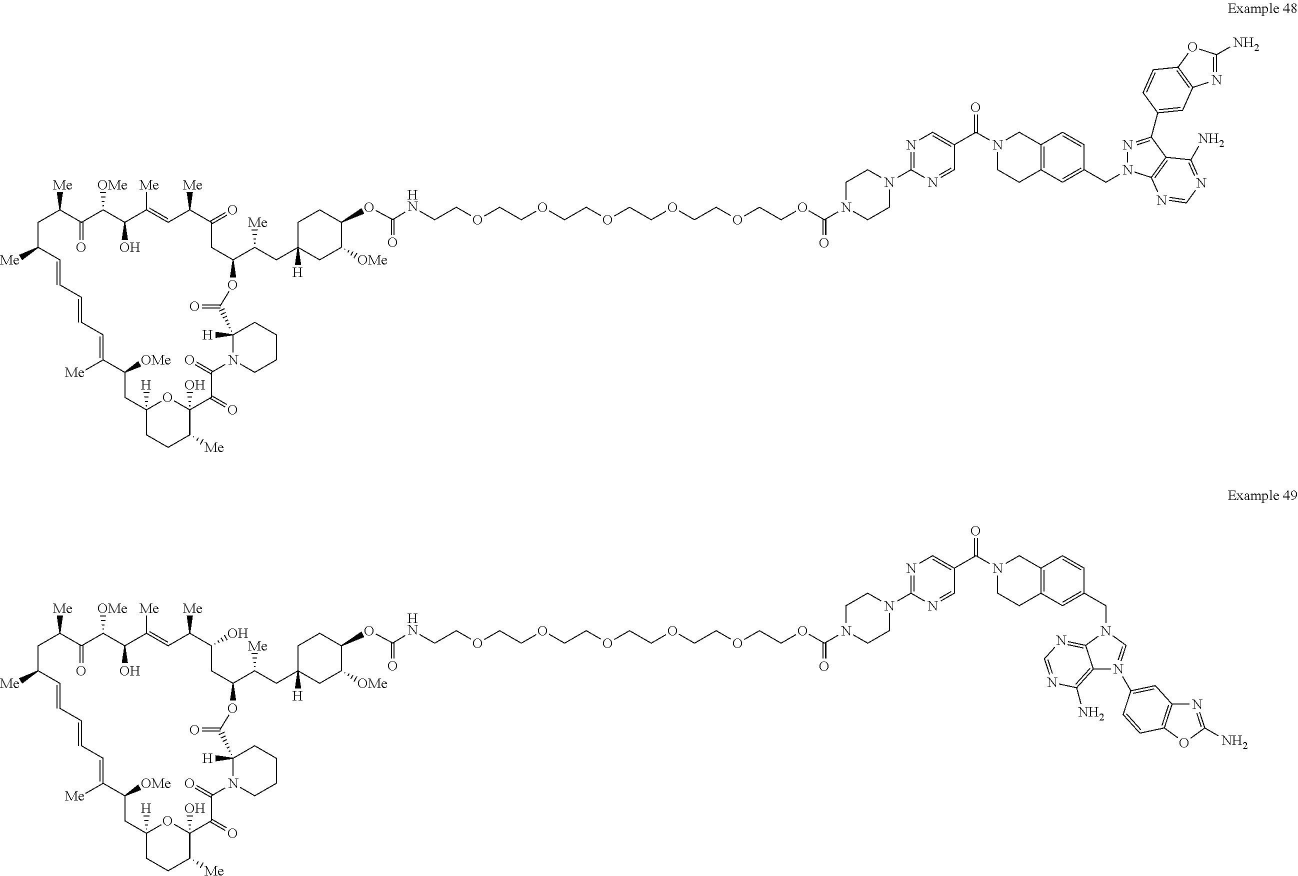

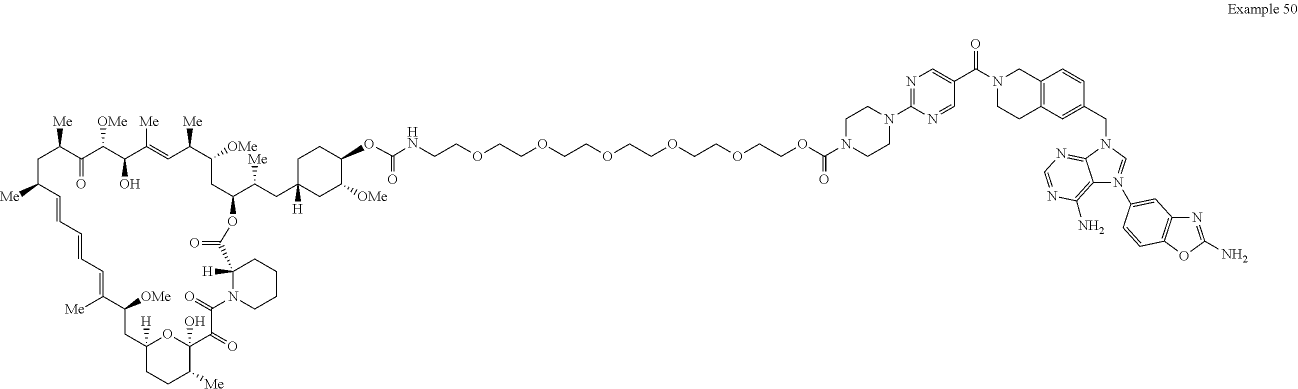

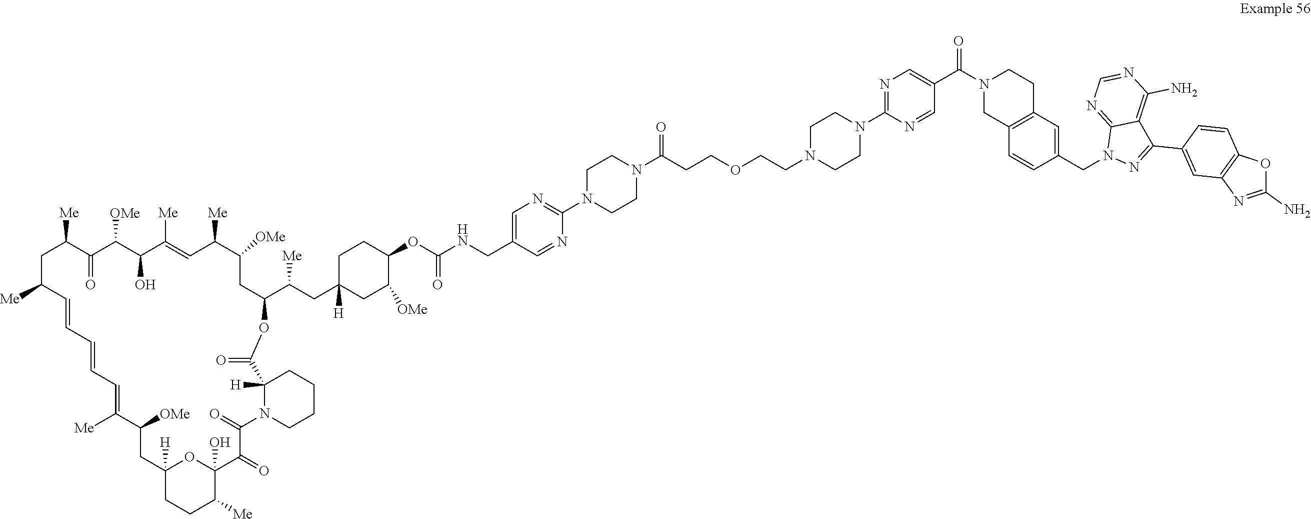 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

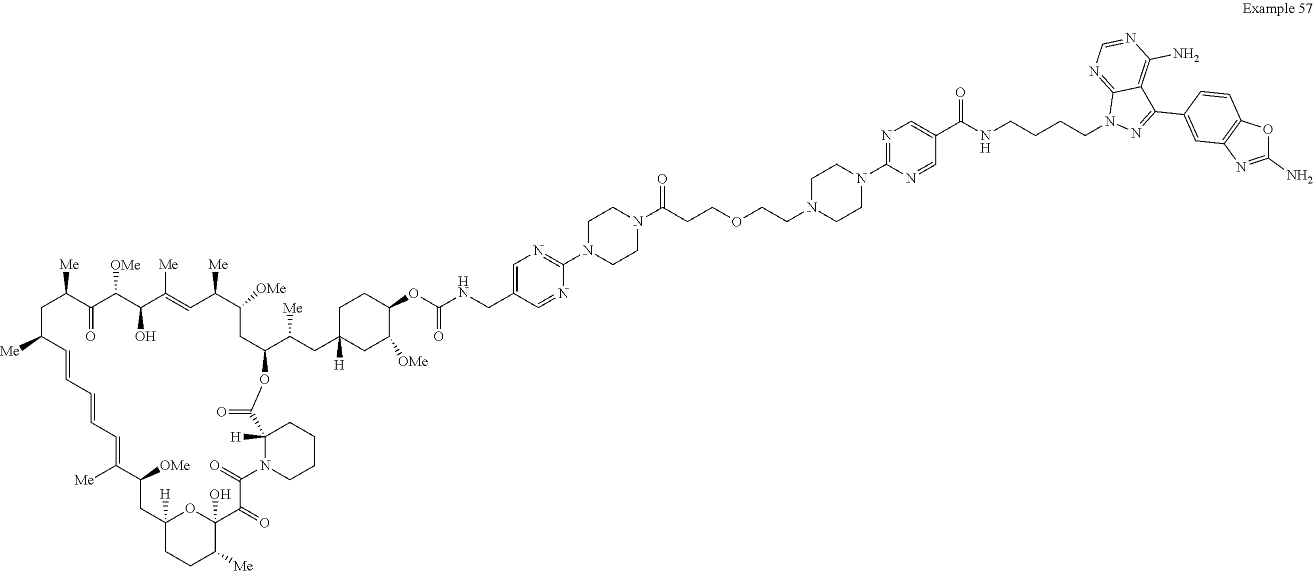 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

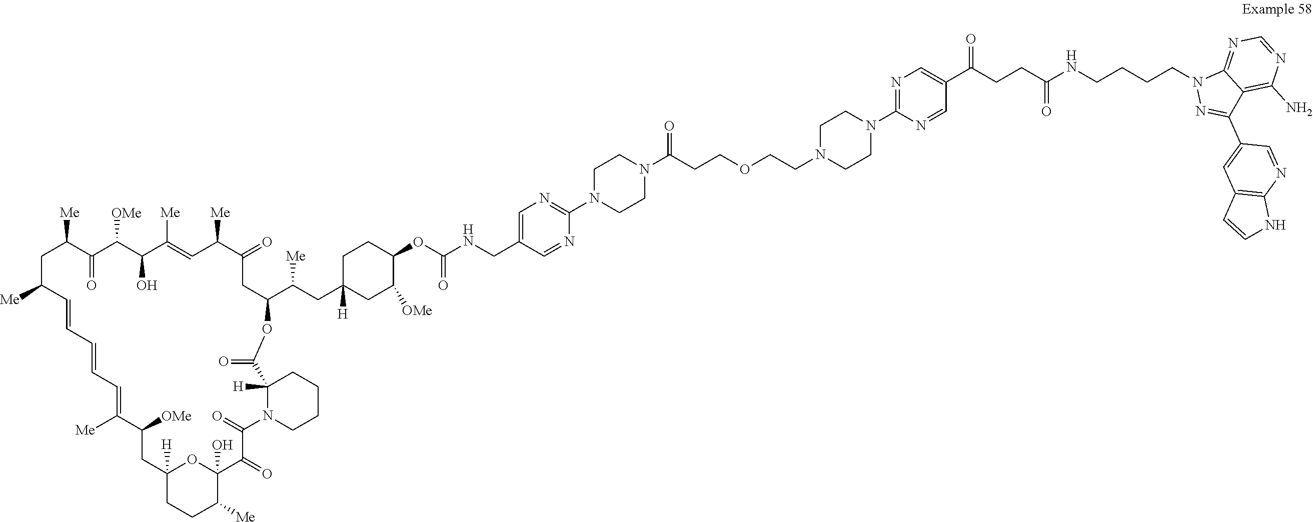 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)CCC(=O)NCCCCn7nc(-c8cnc9[nH]ccc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

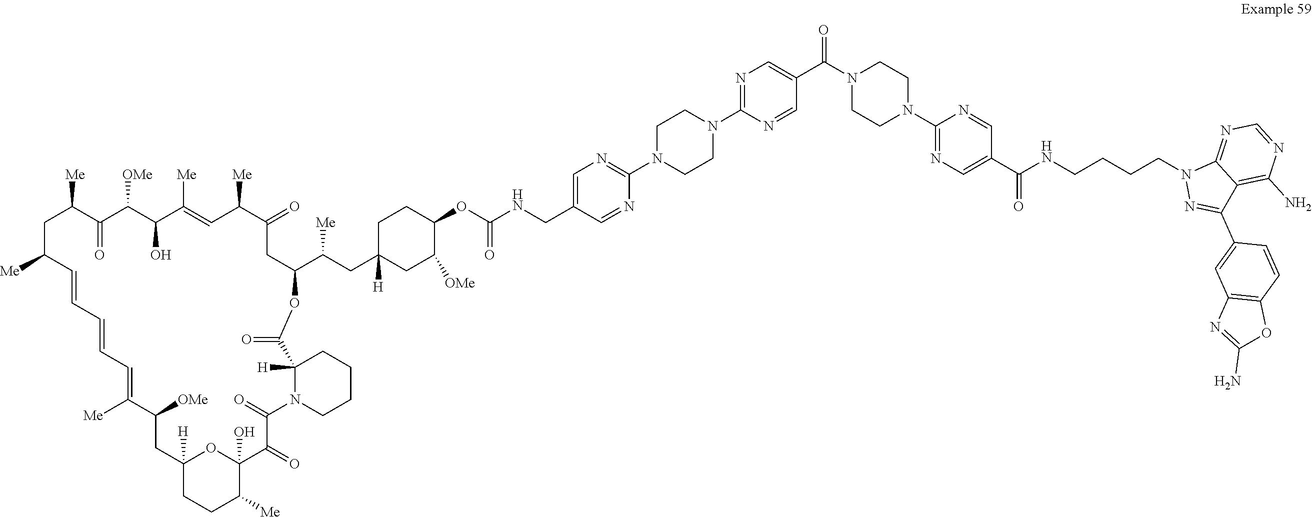 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

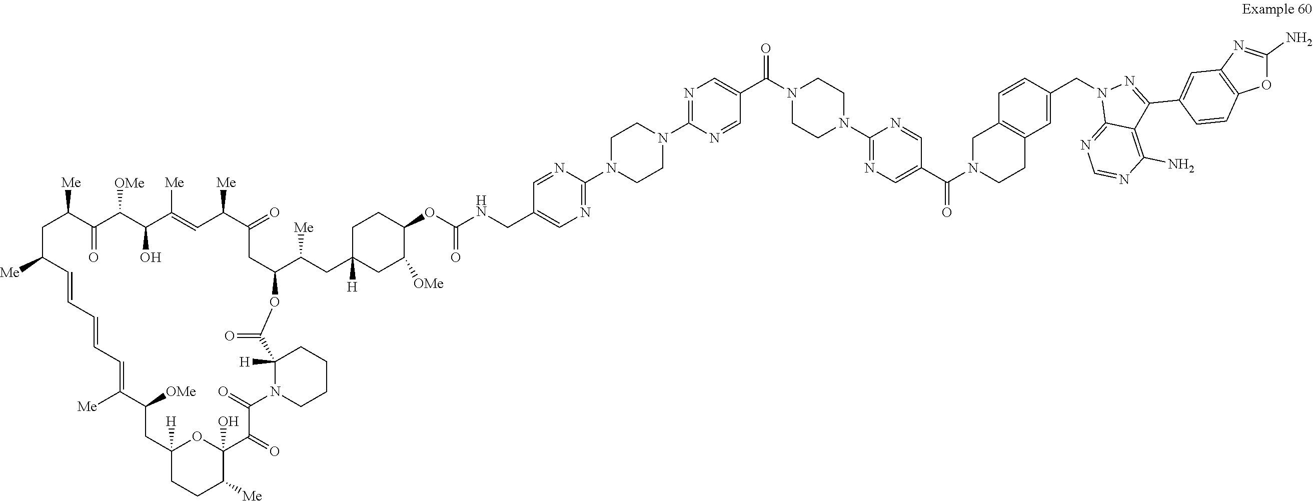

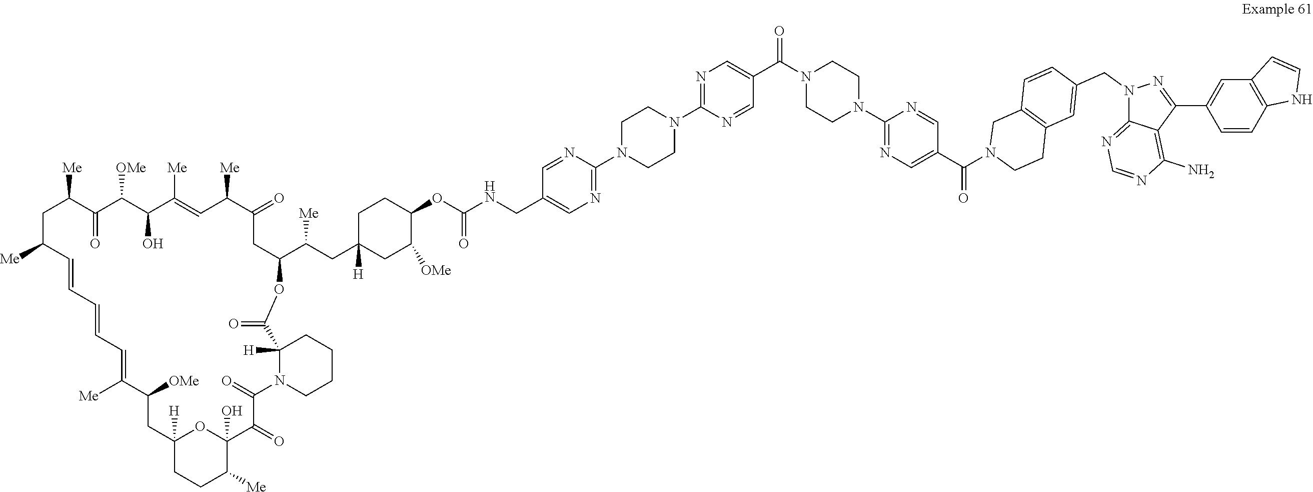

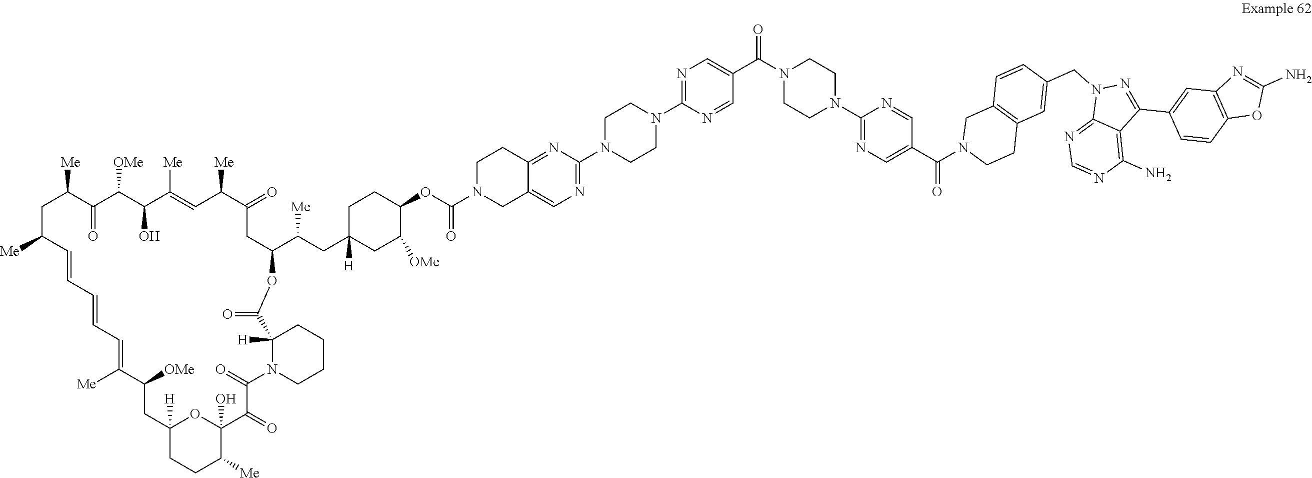

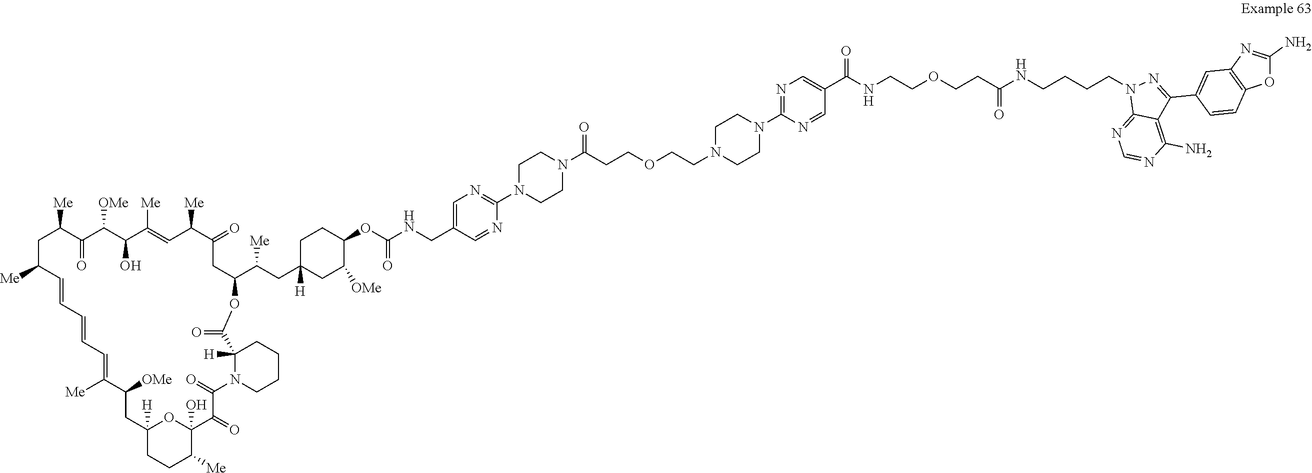

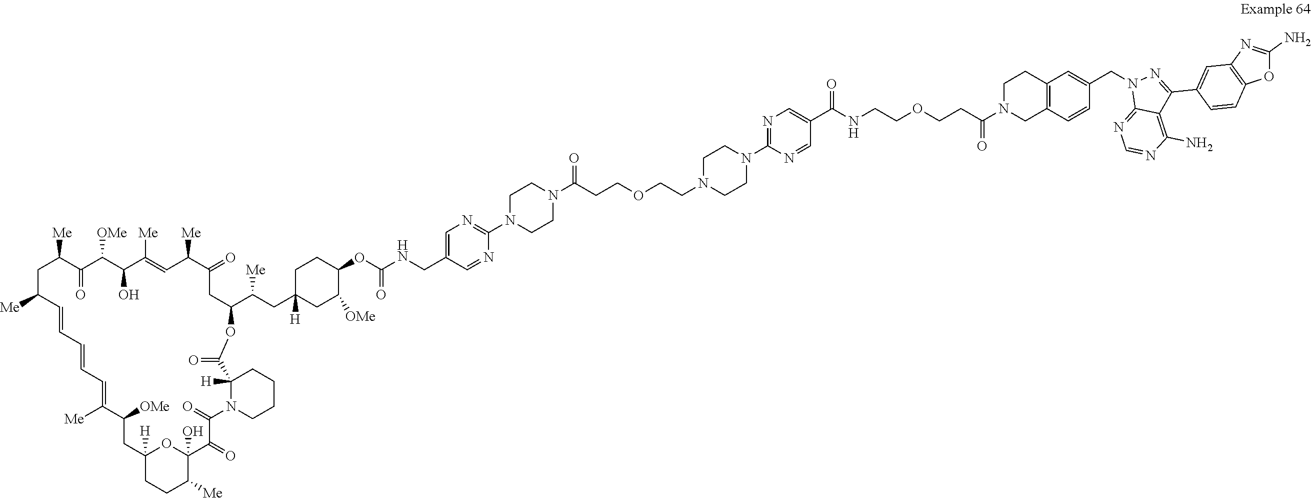 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

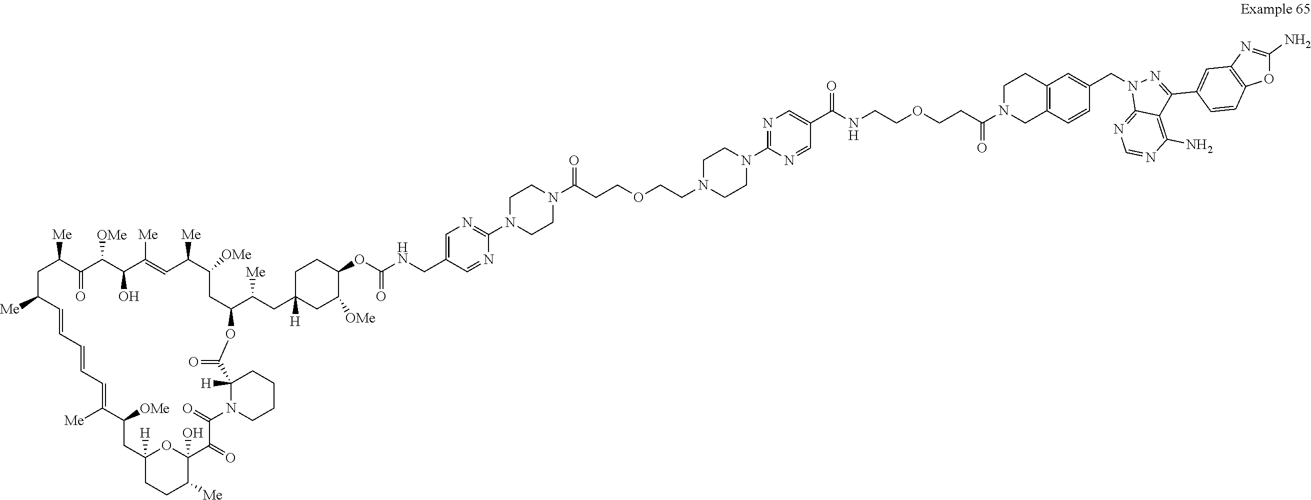

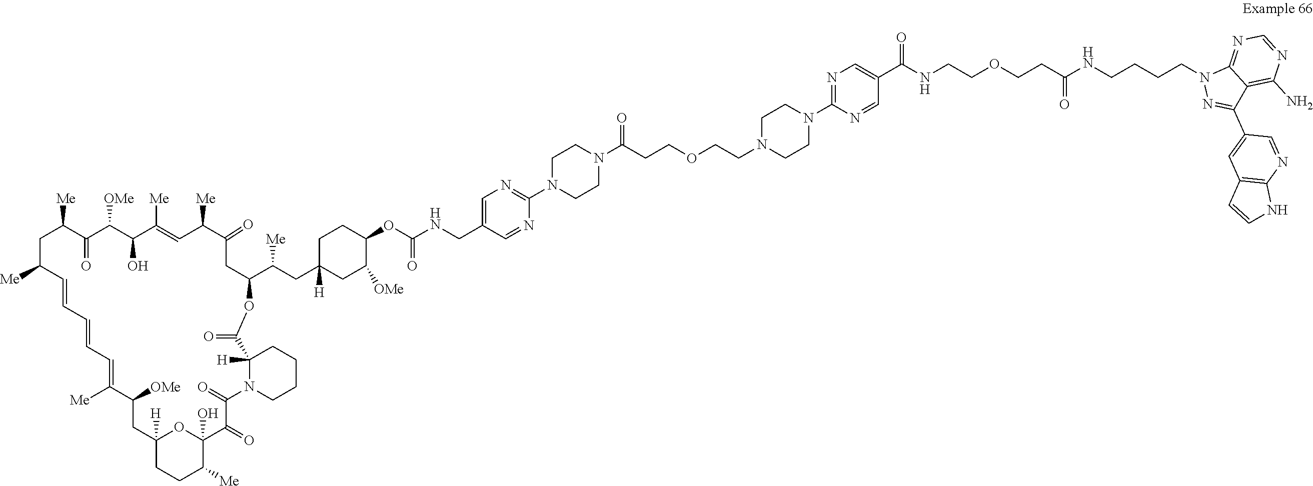

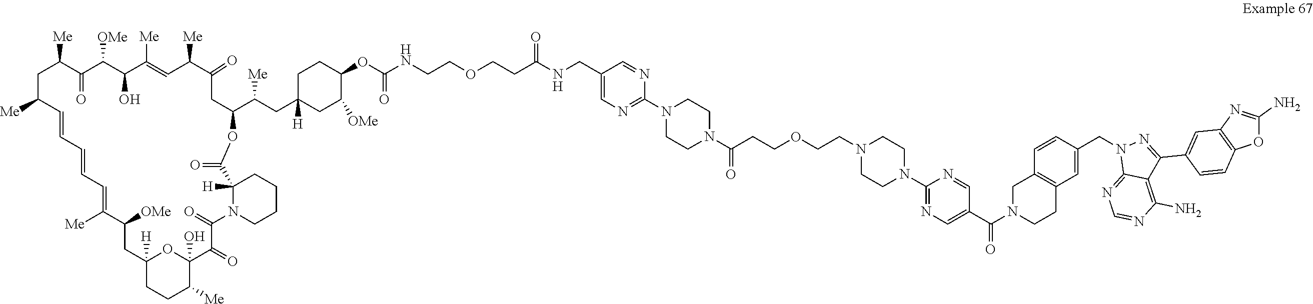 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

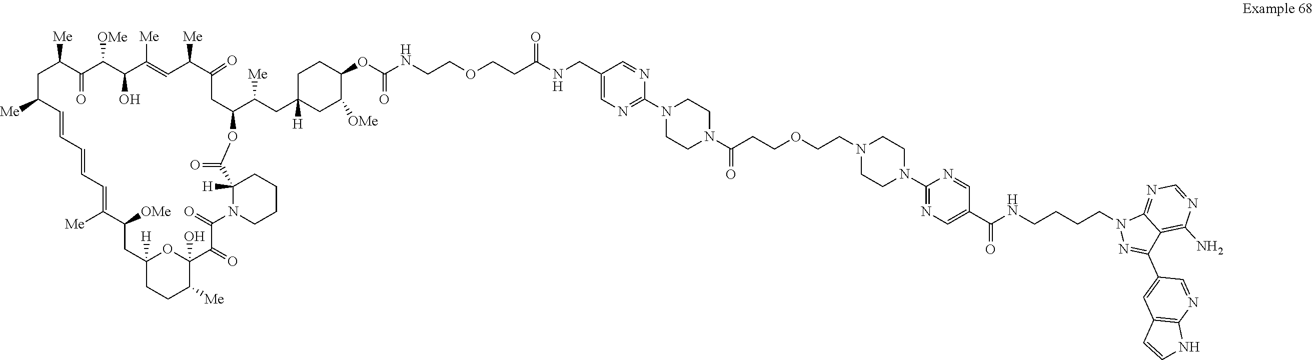

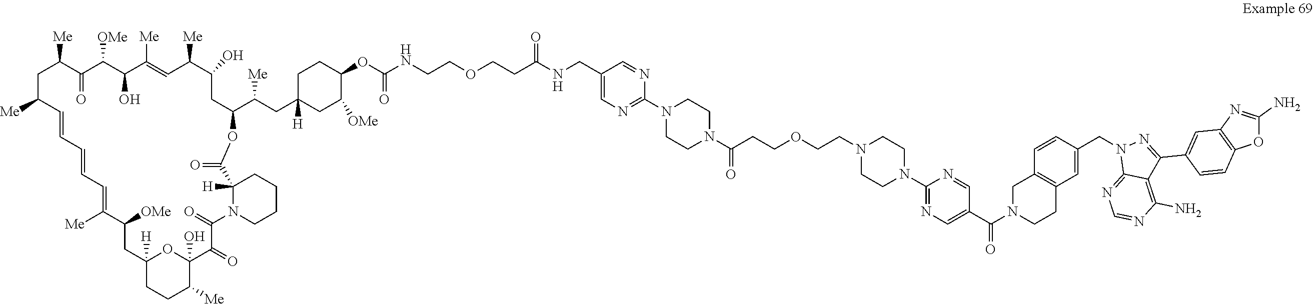 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

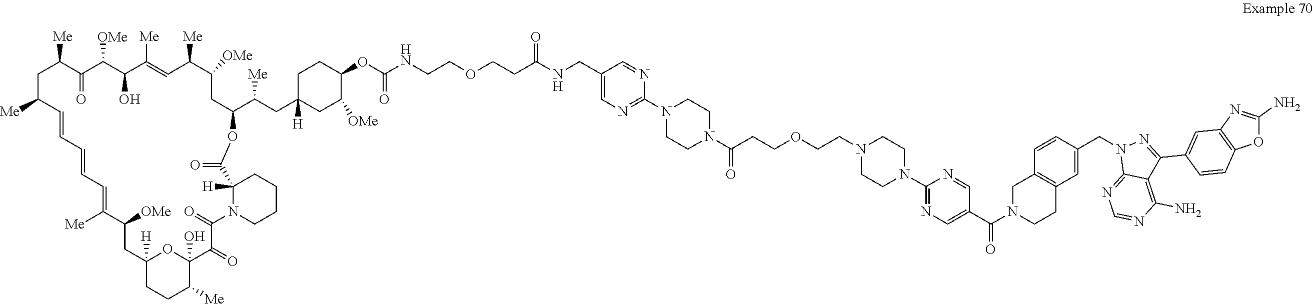 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

or a pharmaceutically acceptable salt or isomer thereof.

Embodiment I-61. A pharmaceutical composition comprising a compound of any one of Embodiments I-1 to I-60, or a pharmaceutically acceptable salt thereof, and at least one of a pharmaceutically acceptable carrier, diluent, or excipient.

Embodiment I-62. A method of treating a disease or disorder mediated by mTOR comprising administering to the subject suffering from or susceptible to developing a disease or disorder mediated by mTOR a therapeutically effective amount of one or more compounds of any one of Embodiments I-1 to I-60, or a pharmaceutically acceptable salt thereof.

Embodiment I-63. A method of preventing a disease or disorder mediated by mTOR comprising administering to the subject suffering from or susceptible to developing a disease or disorder mediated by mTOR a therapeutically effective amount of one or more compounds of any one of Embodiments I-1 to I-60, or a pharmaceutically acceptable salt thereof.

Embodiment I-64. A method of reducing the risk of a disease or disorder mediated by mTOR comprising administering to the subject suffering from or susceptible to developing a disease or disorder mediated by mTOR a therapeutically effective amount of one or more compounds of any one of Embodiments I-1 to I-60, or a pharmaceutically acceptable salt thereof.

Embodiment I-65. The method of any one of Embodiments I-62 to I-64, wherein the disease is cancer or an immune-mediated disease.

Embodiment I-66. The method of Embodiment I-65, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment I-67. The method of Embodiment I-65, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment I-68. A method of treating cancer comprising administering to the subject a therapeutically effective amount of one or more compounds of any one of Embodiments I-1 to I-60, or a pharmaceutically acceptable salt thereof.

Embodiment I-69. The method of Embodiment I-68, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment I-70. A method of treating an immune-mediated disease comprising administering to the subject a therapeutically effective amount of one or more compounds of any one of Embodiments I-1 to I-60, or a pharmaceutically acceptable salt thereof.

Embodiment I-71. The method of Embodiment I-70, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment I-72. A method of treating an age related condition comprising administering to the subject a therapeutically effective amount of one or more compounds of any one of Embodiments I-1 to I-60, or a pharmaceutically acceptable salt thereof.

Embodiment I-73. The method of Embodiment I-72, wherein the age related condition is selected from sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes.

Embodiment I-74. A compound of any one of Embodiments I-1 to I-60, or a pharmaceutically acceptable salt thereof, for use in treating, preventing, or reducing the risk of a disease or condition mediated by mTOR.

Embodiment I-75. Use of a compound of any of Embodiments I-1 to I-60, or a pharmaceutically acceptable salt thereof, in the manufacture of a medicament for treating, preventing, or reducing the risk of a disease or disorder mediated by mTOR.

Embodiment I-76. A compound of any one of Embodiments I-1 to I-60, or a pharmaceutically acceptable salt thereof, for use in treating cancer.

Embodiment I-77. Use of a compound of any one of Embodiments I-1 to I-60, or a pharmaceutically acceptable salt thereof, in the manufacture of a medicament for treating cancer.

Embodiment I-78. A compound of any one of Embodiments I-1 to I-60, or a pharmaceutically acceptable salt thereof, for use in treating an immune-mediated disease.

Embodiment I-79. Use of a compound of any one of Embodiments I-1 to I-60, or a pharmaceutically acceptable salt thereof, in the manufacture of a medicament for treating an immune-mediated disease.

Embodiment I-80. A compound of any one of Embodiments I-1 to I-60, or a pharmaceutically acceptable salt thereof, for use in treating an age related condition.

Embodiment I-81. Use of a compound of any one of Embodiments I-1 to I-60, or a pharmaceutically acceptable salt thereof, in the manufacture of a medicament for treating an age related condition.

Some embodiments of this disclosure are Embodiment II, as follows:

Embodiment II-1. A compound of Formula Ic:

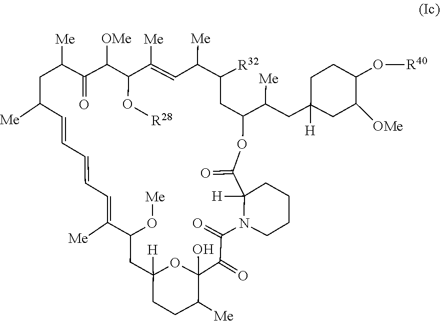

or a pharmaceutically acceptable salt or tautomer thereof, wherein:
R³² is —H, ═O, —OR³, —N₃, or —O—C(═Z¹)—R^{32a};
R²⁸ is —H, (C₁-C₆)alkyl, or —C(═Z¹)—R^{28a};
R⁴⁰ is —H or —C(═Z¹)—R^{40a};
   wherein when R²⁸ and R⁴⁰ are H, then R³² is not ═O;
each Z¹ is independently O or S;
R^{28a}, R^{32a}, and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;
A¹ and A² are independently absent or are independently selected from

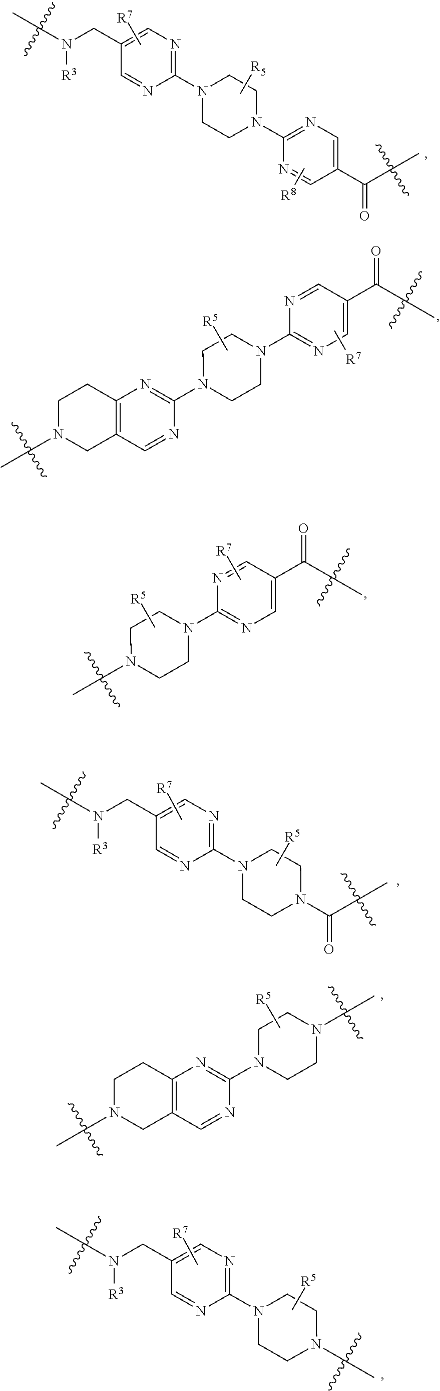

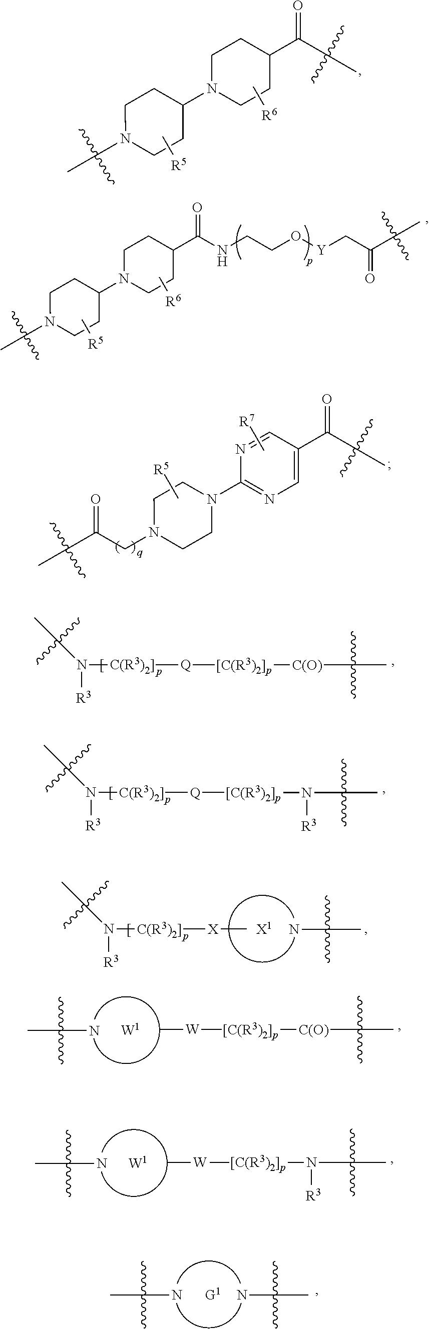

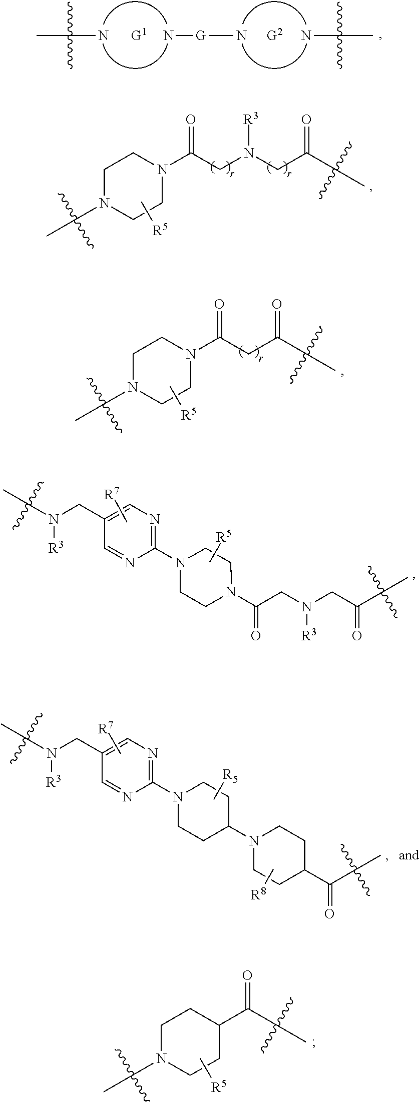

wherein the bond on the left side of A¹, as drawn, is bound to —C(═Z¹)— or L²; and wherein the bond on the right side of the A² moiety, as drawn, is bound to B or L³;
each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ is independently a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;
each L¹ is independently selected from

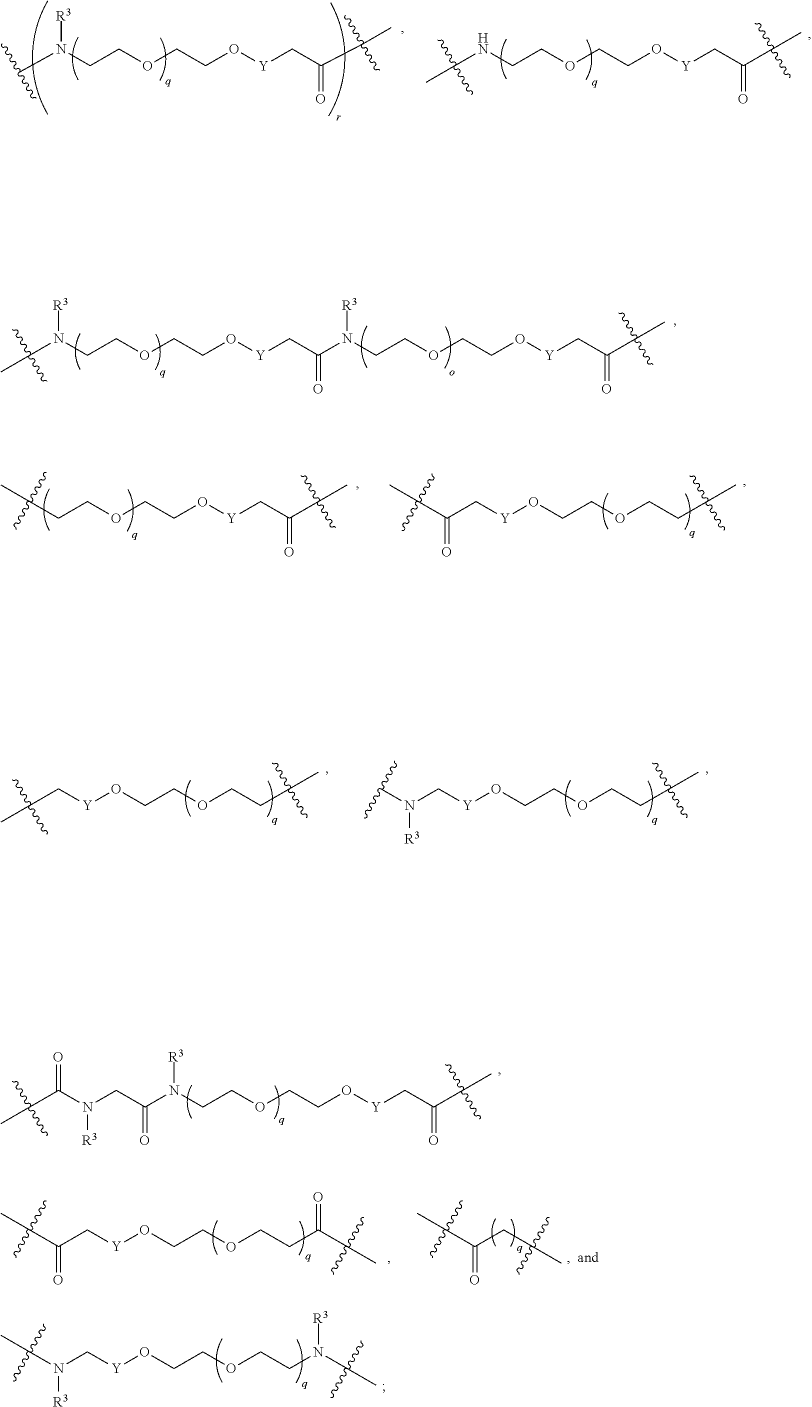

L² and L³ are independently absent or are independently selected from

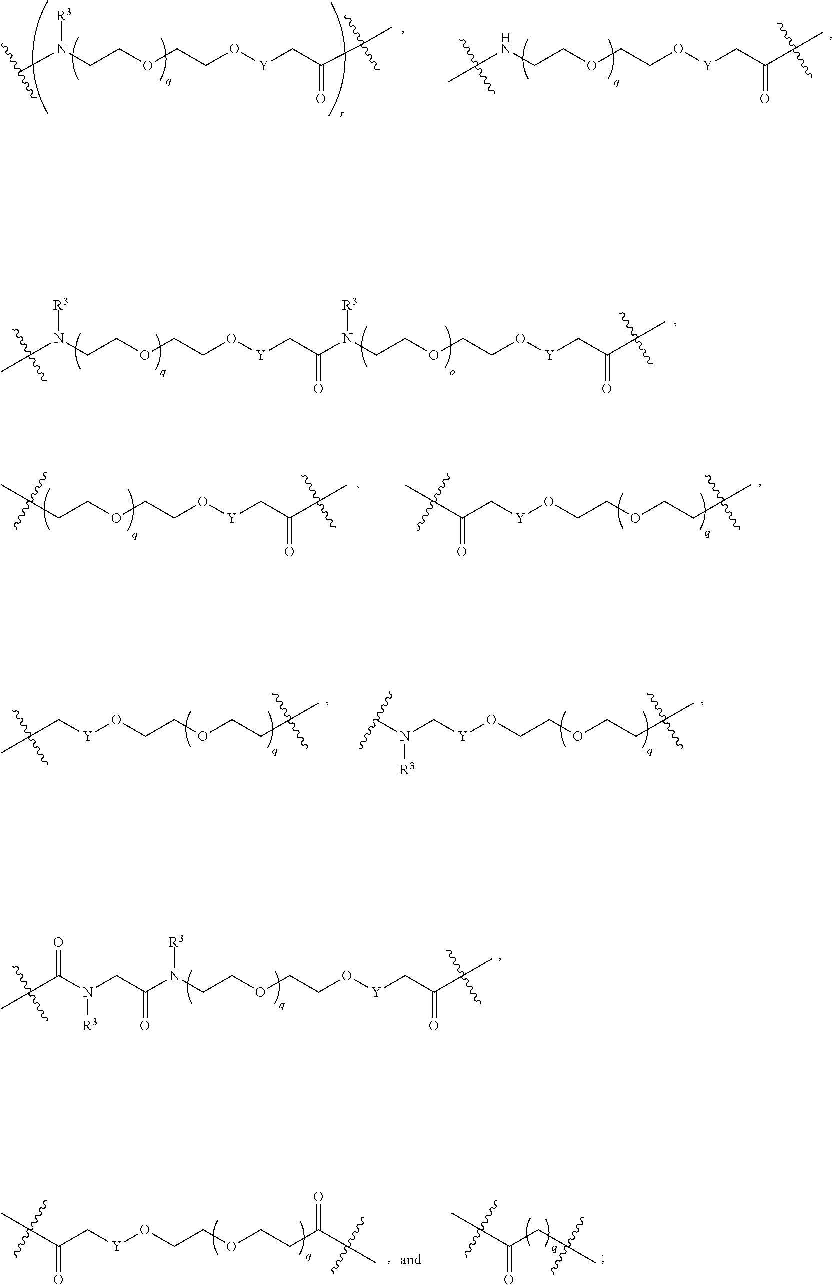

each B is independently selected from

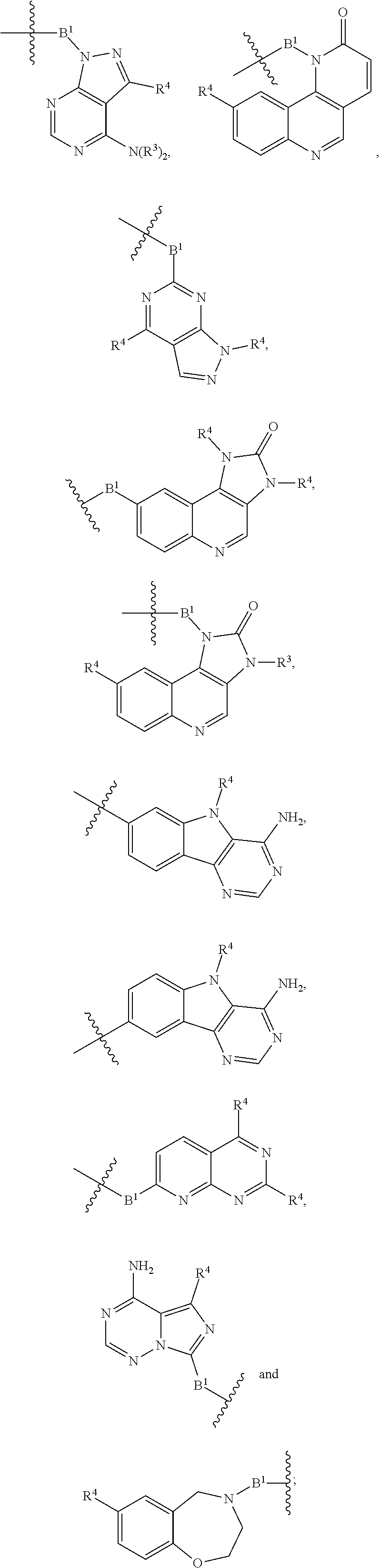

each B¹ is independently selected from

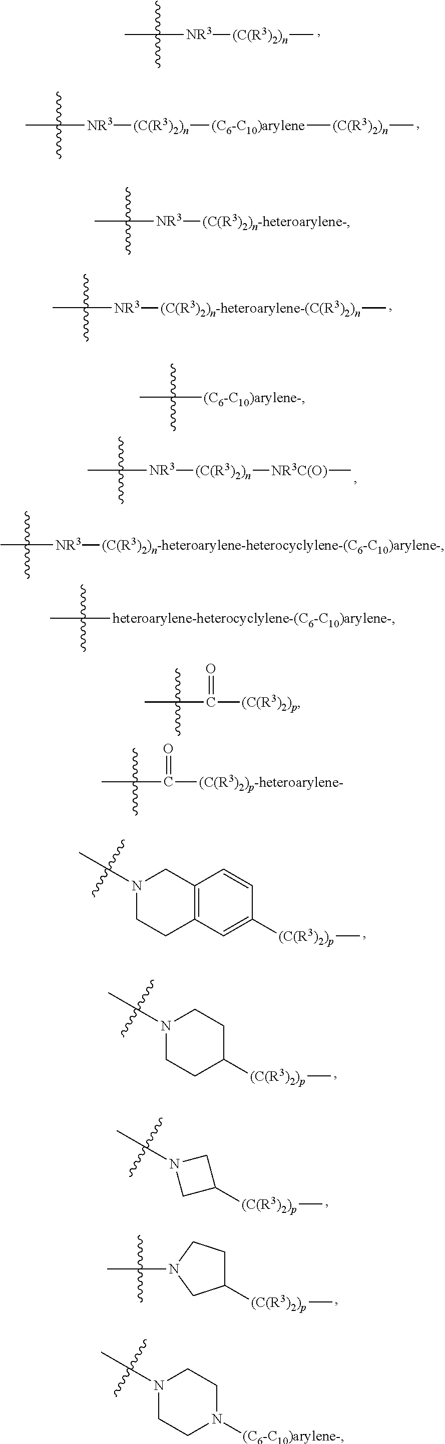

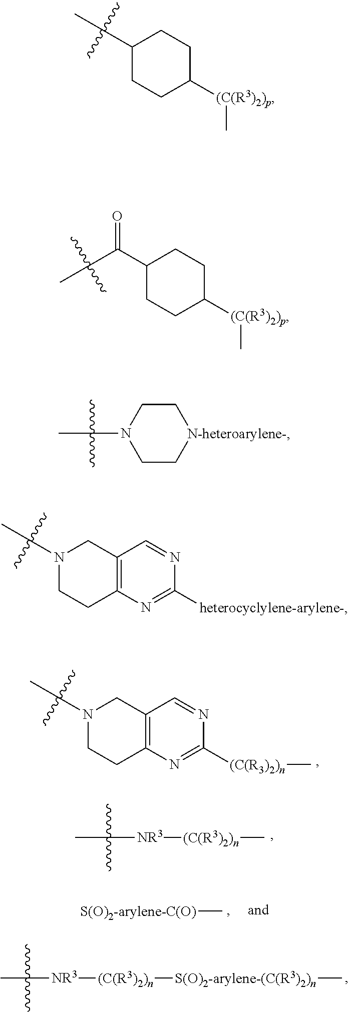

wherein the

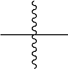

bond on the left side of B¹, as drawn, is bound to A², L³, or L¹; and wherein the heteroarylene, heterocyclylene, and arylene are each independently optionally substituted with alkyl, hydroxyalkyl, haloalkyl, alkoxy, halogen, or hydroxyl;
each R³ is independently H or (C₁-C₆)alkyl;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;
each R⁵ is independently H, (C₁-C₆)alkyl, —C(O)OR³, or —N(R³)₂, wherein the alkyl of (C₁-C₆)alkyl is optionally substituted with —N(R³)₂ or —OR³;
each R⁶ is independently H, (C₁-C₆)alkyl, —C(O)OR³, or —N(R³)₂, wherein the alkyl of (C₁-C₆)alkyl is optionally substituted with —N(R³)₂ or —OR³;
each R⁷ is independently H, (C₁-C₆)alkyl, —C(O)OR³, or —N(R³)₂, wherein the alkyl of (C₁-C₆)alkyl is optionally substituted with —N(R³)₂ or —OR³;
each R⁸ is independently H, (C₁-C₆)alkyl, —C(O)OR³, or —N(R³)₂, wherein the alkyl of (C₁-C₆)alkyl is optionally substituted with —N(R³)₂ or —OR³;
each Y is independently C(R³)₂ or a bond;
each n is independently an integer from one to 12;
each o is independently an integer from zero to 30;
each p is independently an integer from zero to 12;
each q is independently an integer from zero to 30; and
each r is independently an integer from one to 6.

Embodiment II-1A. A compound of Formula Ia:

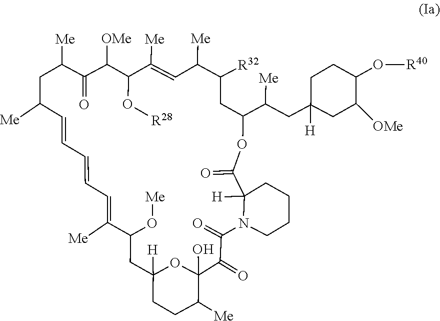

or a pharmaceutically acceptable salt or tautomer thereof, wherein:
R³² is —H, ═O, —OR³, —N₃, or —O—C(═Z¹)—R^{32a};
R²⁸ is —H, (C₁-C₆)alkyl, or —C(═Z¹)—R^{28a};
R⁴⁰ is —H or —C(═Z¹)—R^{40a};
   wherein when R²⁸ and R⁴⁰ are H, then R³² is not ═O;
each Z¹ is independently O or S;
R^{28a}, R^{32a}, and R^{40a} are independently -A¹-L¹-A²-B; -A¹-A²-B; -L²-A¹-L¹-A²-L³-B; —O—(C₁-C₆)alkyl; or —O—(C₆-C₁₀)aryl; wherein the aryl of —O—(C₆-C₁₀)aryl is unsubstituted or substituted with 1-5 substituents selected from —NO₂ and halogen;
A¹ and A² are independently absent or are independently selected from

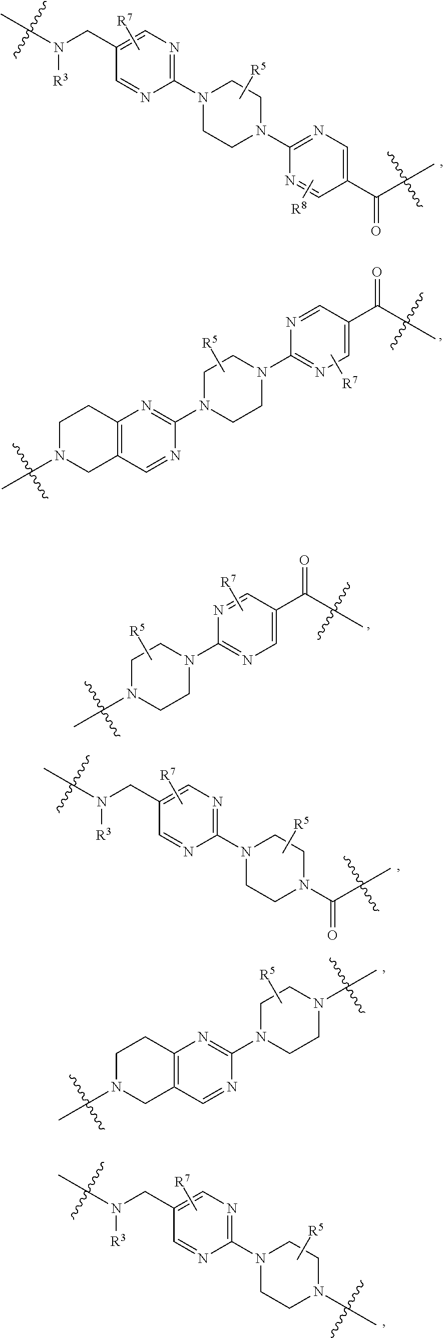

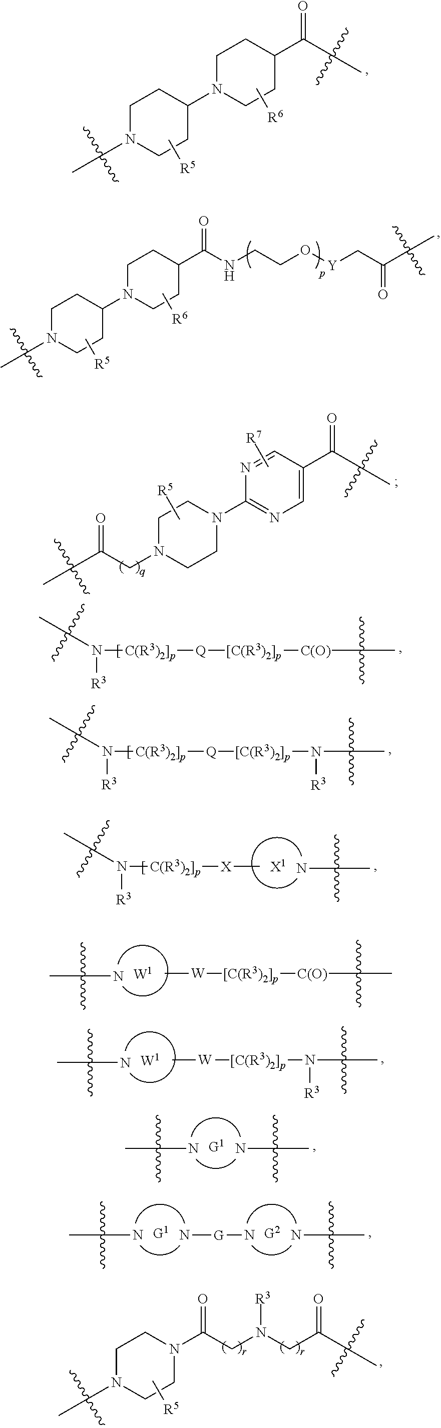

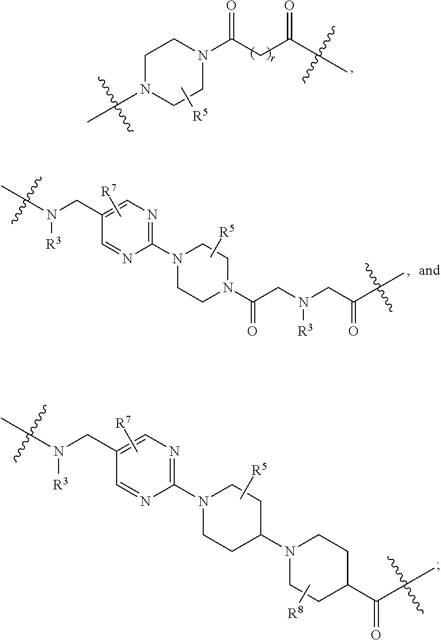

wherein the bond on the left side of A¹, as drawn, is bound to —C(═Z¹)— or L²; and wherein the bond on the right side of the A² moiety, as drawn, is bound to B or L³;
each Q is independently 1 to 3 rings selected from arylene, cycloalkylene, heteroarylene, and heterocyclylene;
each X is independently absent or 1 to 2 rings selected from arylene, cycloalkylene, heteroarylene, and heterocyclylene;
each X¹ is independently a heteroarylene or heterocyclylene ring;
each W is independently absent or 1 to 2 rings selected from arylene, cycloalkylene, heteroarylene, and heterocyclylene;
each W¹ is independently a heteroarylene or heterocyclylene ring;
each G is independently absent or a ring selected from arylene, cycloalkylene, heteroarylene, and heterocyclylene;
each G¹ and G² are independently heteroarylene or heterocyclylene ring;
each L¹ is independently selected from

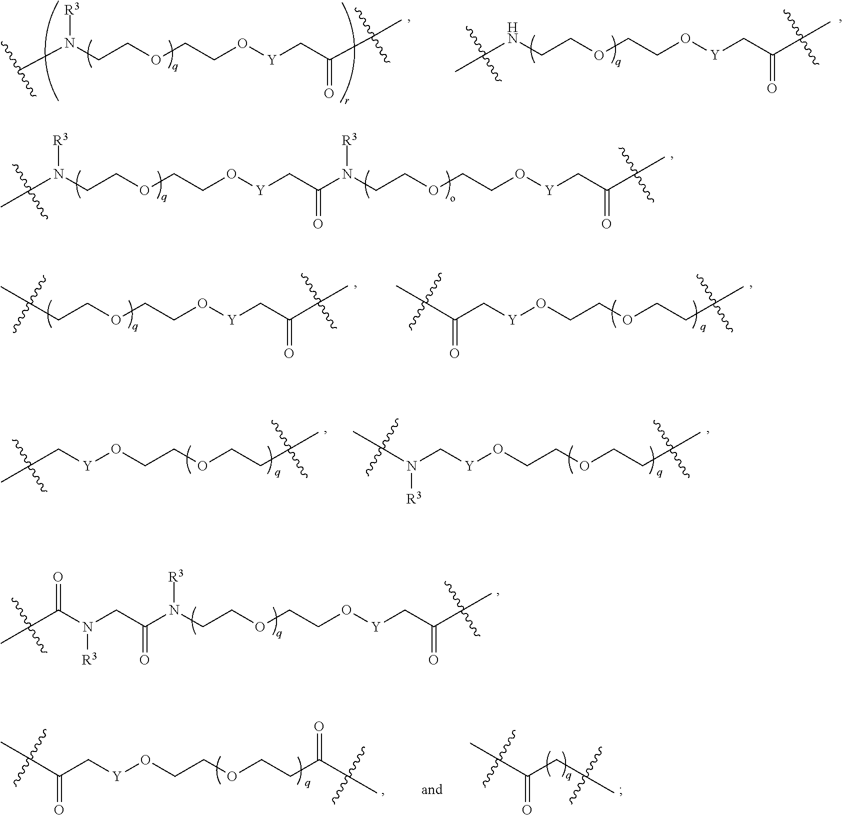

L² and L³ are independently absent or are independently selected from

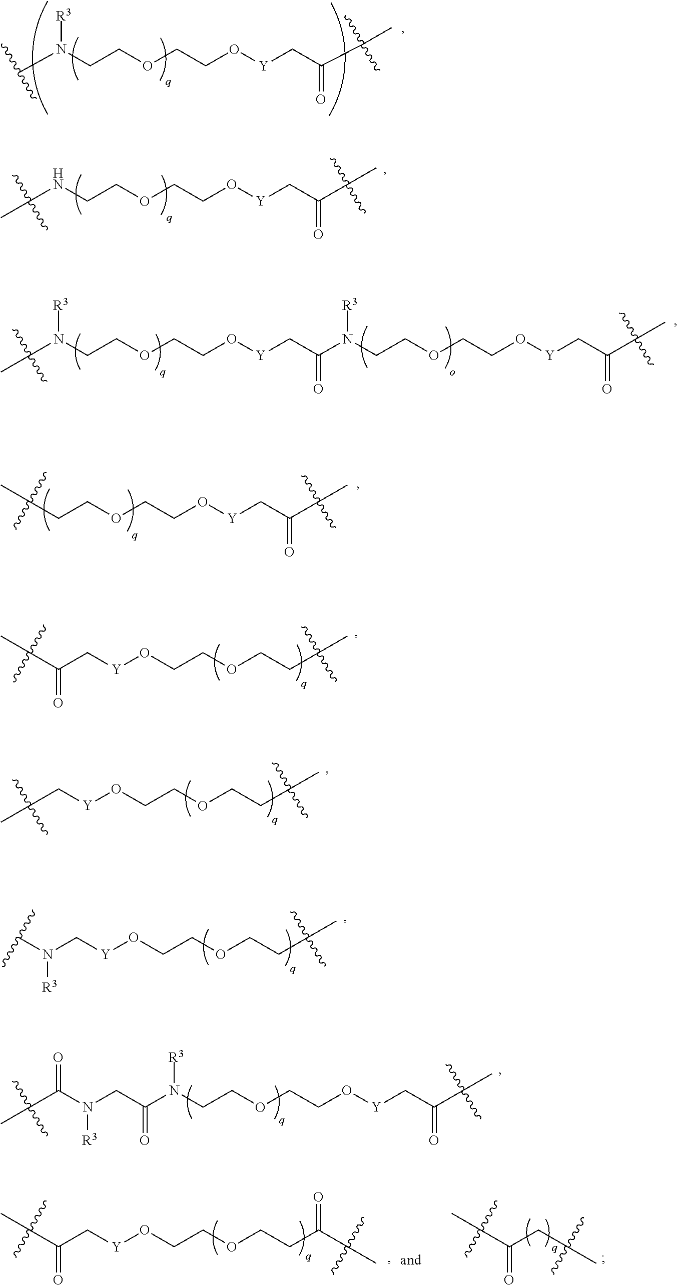

each B is independently selected from

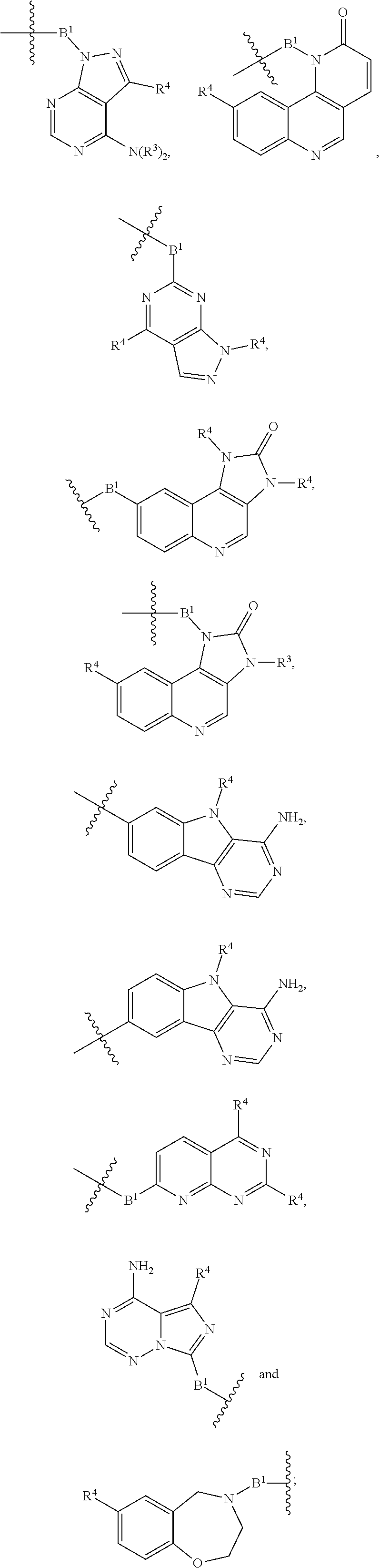

B¹ is selected from

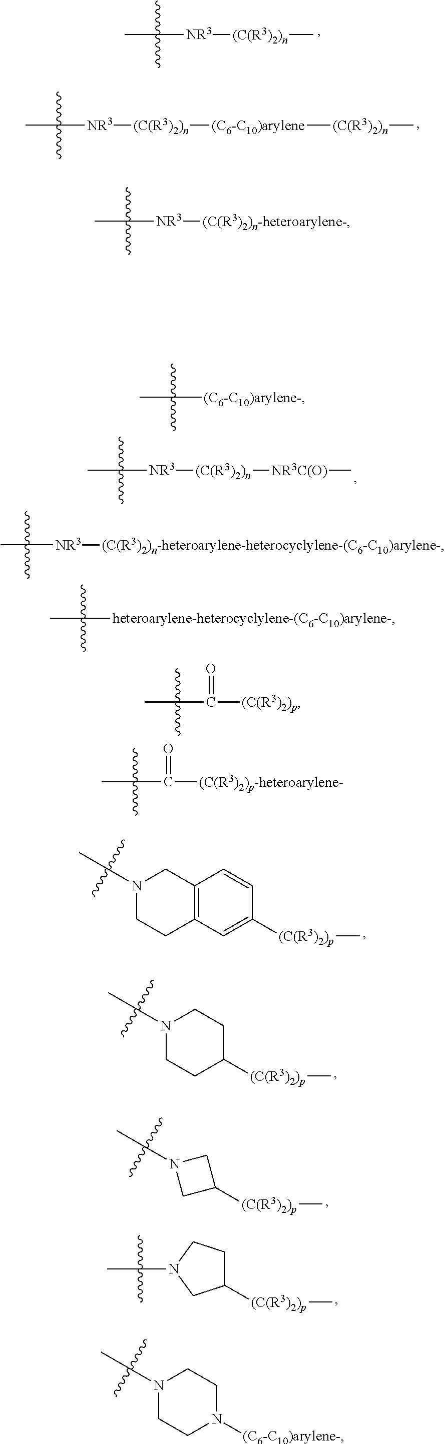

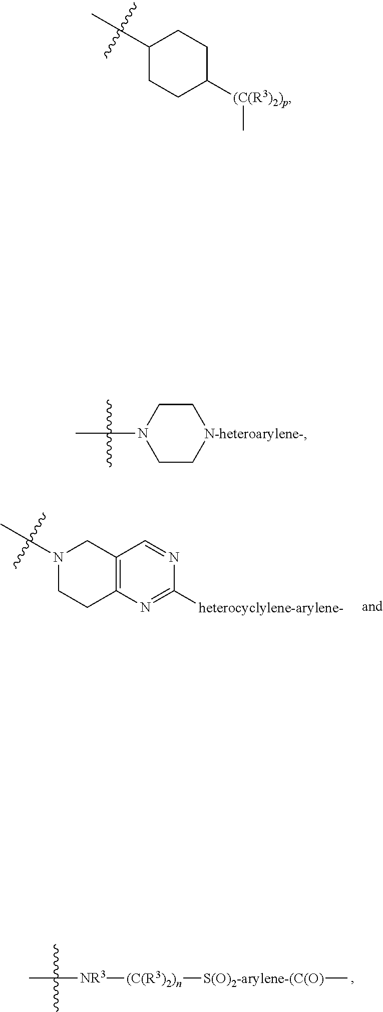

wherein the

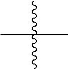

bond on the left side of B¹, as drawn, is bound to A², L³, or L¹; and wherein the heteroarylene, heterocyclylene, and arylene are optionally substituted with alkyl, hydroxyalkyl, haloalkyl, alkoxy, halogen, or hydroxyl;
each R³ is independently H or (C₁-C₆)alkyl;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;
each R⁵ is independently H, (C₁-C₆)alkyl, —C(O)OR³, or —N(R³)₂, wherein the alkyl of (C₁-C₆)alkyl is optionally substituted with —N(R³)₂ or —OR³;
each R⁶ is independently H, (C₁-C₆)alkyl, —C(O)OR³, or —N(R³)₂, wherein the alkyl of (C₁-C₆)alkyl is optionally substituted with —N(R³)₂ or —OR³;
each R⁷ is independently H, (C₁-C₆)alkyl, —C(O)OR³, or —N(R³)₂, wherein the alkyl of (C₁-C₆)alkyl is optionally substituted with —N(R³)₂ or —OR³;
each R⁸ is independently H, (C₁-C₆)alkyl, —C(O)OR³, or —N(R³)₂, wherein the alkyl of (C₁-C₆)alkyl is optionally substituted with —N(R³)₂ or —OR³;
each Y is independently C(R³)₂ or a bond;
each n is independently an integer from one to 12;
each o is independently an integer from zero to 30;
each p is independently an integer from zero to 12;
each q is independently an integer from zero to 30; and
each r is independently an integer from one to 6.

Embodiment II-2. The compound of Embodiment II-1, wherein R³² is ═O.

Embodiment II-3. The compound of Embodiment II-1, wherein R³² is —OR³.

Embodiment II-4. The compound of any one of Embodiments II-1 to II-3, or a pharmaceutically acceptable salt or tautomer thereof, wherein the compound is represented by the structure of Formula (I-40b):

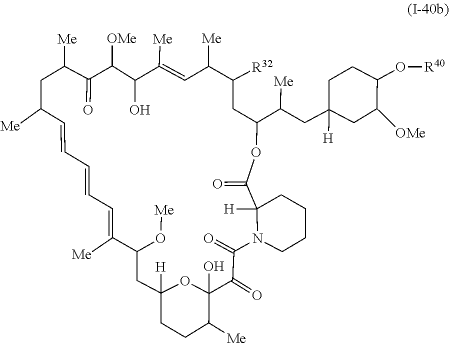

wherein R⁴⁰ is —C(═Z¹)—R^{40a}.

Embodiment II-5. The compound of Embodiment II-4, wherein Z¹ is O.

Embodiment II-6. The compound of Embodiment II-4, wherein Z¹ is S.

Embodiment II-7. The compound of any one of Embodiments II-4 to II-6, wherein R^{40a} is -A¹-L¹-A²-B, wherein A¹ and A² are absent.

Embodiment II-8. The compound of any one of Embodiments II-4 to II-6, wherein R^{40a} is -A¹-L¹-A²-B, wherein A² is absent.

Embodiment II-9. The compound of any one of Embodiments II-4 to II-6, wherein R^{40a} is -A¹-L¹-A²-B, wherein A¹ is absent.

Embodiment II-10. The compound of any one of Embodiments II-4 to II-6, wherein R^{40a} is -A¹-L¹-A²-B.

Embodiment II-11. The compound of any one of Embodiments II-4 to II-6, wherein R^{40a} is -A¹-A²-B.

Embodiment II-12. The compound of any one of Embodiments II-4 to II-6, wherein R^{40a} is -L²-A¹-L¹-A²-L³-B, wherein L² and A¹ are absent.

Embodiment II-13. The compound of any one of Embodiments II-4 to II-6, wherein R^{40a} is -L²-A¹-L¹-A²-L³-B, wherein L² is absent.

Embodiment II-14. The compound of any one of Embodiments II-4 to II-6, wherein R^{40a} is -L²-A¹-L¹-A²-L³-B, wherein L³ is absent.

Embodiment II-15. The compound of any one of Embodiments II-4 to II-6, wherein R^{40a} is —O—(C₁-C₆)alkyl or —O—(C₆-C₁₀)aryl; wherein the aryl of —O—(C₆-C₁₀)aryl is unsubstituted or substituted with 1-5 substituents selected from —NO₂ and halogen.

Embodiment II-16. The compound of any one of Embodiments II-1 to II-3, or a pharmaceutically acceptable salt or tautomer thereof, wherein the compounds are represented by the structure of Formula (I-28b):

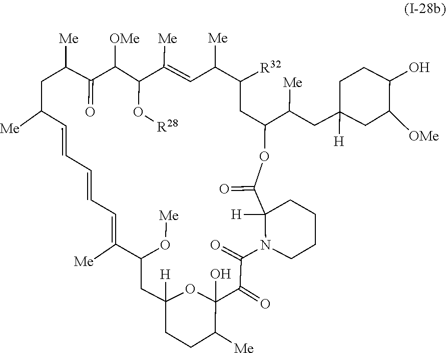

wherein R²⁸ is —C(═Z¹)—R^{28a}.

Embodiment II-17. The compound of Embodiment II-16, wherein Z¹ is O.

Embodiment II-18. The compound of Embodiment II-16, wherein Z¹ is S.

Embodiment II-19. The compound of any one of Embodiments II-16 to II-18, wherein R^{28a} is -A¹-L¹-A²-B, wherein A¹ and A² are absent.

Embodiment II-20. The compound of any one of Embodiments II-16 to II-18, wherein R^{28a} is -A¹-L¹-A²-B, wherein A² is absent.

Embodiment II-21. The compound of any one of Embodiments II-16 to II-18, wherein R^{28a} is -A¹-L¹-A²-B, wherein A¹ is absent.

Embodiment II-22. The compound of any one of Embodiments II-16 to II-18, wherein R^{28a} is -A¹-L¹-A²-B.

Embodiment II-23. The compound of any one of Embodiments II-16 to II-18, wherein R^{28a} is -A¹-A²-B.

Embodiment II-24. The compound of any one of Embodiments II-16 to II-18, wherein R^{28a} is -L²-A¹-L¹-A²-L³-B, wherein L² and A¹ are absent.

Embodiment II-25. The compound of any one of Embodiments II-16 to II-18, wherein R^{28a} is -L²-A¹-L¹-A²-L³-B, wherein L² is absent.

Embodiment II-26. The compound of any one of Embodiments II-16 to II-18, wherein R^{28a} is -L²-A¹-L¹-A²-L³-B, wherein L³ is absent.

Embodiment II-27. The compound of any one of Embodiments II-16 to II-18, wherein R^{28a} is —O—(C₁-C₆)alkyl or —O—(C₆-C₁₀)aryl; wherein the aryl of —O—(C₆-C₁₀)aryl is unsubstituted or substituted with 1-5 substituents selected from —NO₂ and halogen.

Embodiment II-28. The compound of Embodiment II-1, or a pharmaceutically acceptable salt or tautomer thereof, wherein the compound is represented by the structure of Formula (I-32b):

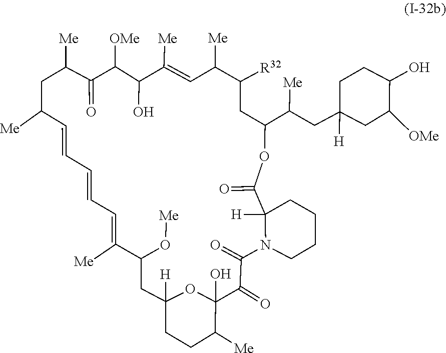

wherein R³² is —O—C(═Z¹)—R^{32a}.

Embodiment II-29. The compound of Embodiment II-28, wherein Z¹ is O.

Embodiment II-30. The compound of Embodiment II-28, wherein Z¹ is S.

Embodiment II-31. The compound of any one of Embodiments II-28 to II-30, wherein R^{32a} is -A¹-L¹-A²-B, wherein A¹ and A² are absent.

Embodiment II-32. The compound of any one of Embodiments II-28 to II-30, wherein R^{32a} is -A¹-L¹-A²-B, wherein A² is absent.

Embodiment II-33. The compound of any one of Embodiments II-28 to II-30, wherein R^{32a} is -A¹-L¹-A²-B, wherein A¹ is absent.

Embodiment II-34. The compound of any one of Embodiments II-28 to II-30, wherein R^{32a} is -A¹-L¹-A²-B.

Embodiment II-35. The compound of any one of Embodiments II-28 to II-30, wherein R^{32a} is -A¹-A²-B.

Embodiment II-36. The compound of any one of Embodiments II-28 to II-30, wherein R^{32a} is -L²-A¹-L¹-A²-L³-B, wherein L² and A¹ are absent.

Embodiment II-37. The compound of any one of Embodiments II-28 to II-30, wherein R^{32a} is -²-A¹-L¹-A²-L³-B, wherein L² is absent.

Embodiment II-38. The compound of any one of Embodiments II-28 to II-30, wherein R^{32a} is -L²-A¹-L¹-A²-L³-B, wherein L³ is absent.

Embodiment II-39. The compound of any one of Embodiments II-28 to II-30, wherein R^{32a} is —O—(C₁-C₆)alkyl or —O—(C₆-C₁₀)aryl; wherein the aryl of —O—(C₆-C₁₀)aryl is unsubstituted or substituted with 1-5 substituents selected from —NO₂ and halogen.

Embodiment II-40. The compound of any one of Embodiments II-1 to II-10, II-12 to II-22, II-24 to II-35, and II-36 to II-39, wherein L¹ is

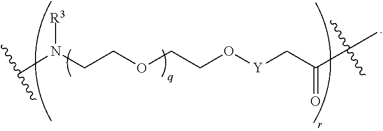

Embodiment II-41. The compound of any one Embodiments II-1 to II-10, II-12 to II-22, II-24 to II-35, and II-36 to II-39, wherein L¹ is

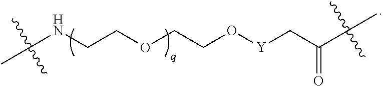

Embodiment II-42. The compound of any one of Embodiments II-1 to II-10, II-12 to II-22, 24-35, and 36-39, wherein L¹ is

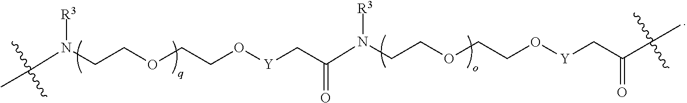

Embodiment II-43. The compound of any one of Embodiments II-1 to II-10, II-12 to II-22, II-24 to II-35, and II-36 to II-39, wherein L¹ is

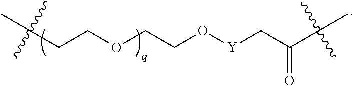

Embodiment II-44. The compound of any one of Embodiments II-1 to II-10, II-12 to II-22, II-24 to II-35, and II-36 to II-39, wherein L¹ is

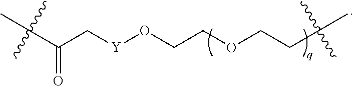

Embodiment II-45. The compound of any one of Embodiments II-1 to II-10, II-12 II-22, II-24 to II-35, and II-36 to II-39, wherein L¹ is

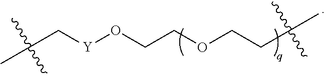

Embodiment II-46. The compound of any one of Embodiments II-1 to II-6, II-12 to II-18, II-24 to II-30, and II-36 to II-45, wherein L² is

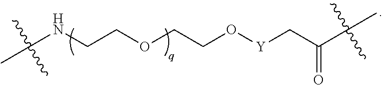

Embodiment II-47. The compound of any one of Embodiments II-1 to II-6, II-12 to II-18, II-24 to II-30, and II-36 to II-45, wherein L³ is

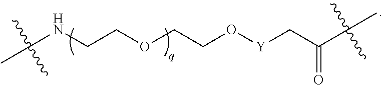

Embodiment II-48. The compound of any one of Embodiments II-1 to II-7, II-9, II-12, II-16 to II-19, II-21, II-24, II-28 to II-31, II-33, II-36, and II-39 to II-45, wherein A¹ is absent.

Embodiment II-49. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

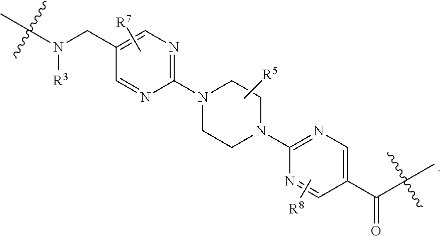

Embodiment II-50. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

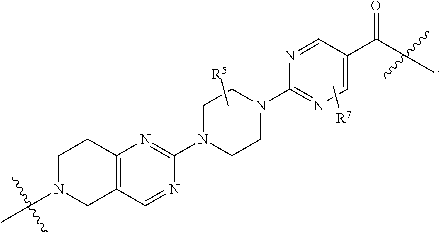

Embodiment II-51. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

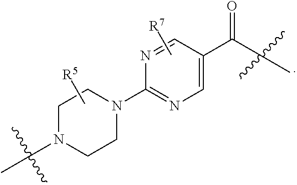

Embodiment II-52. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

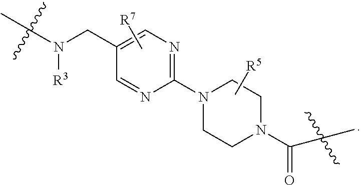

Embodiment II-53. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

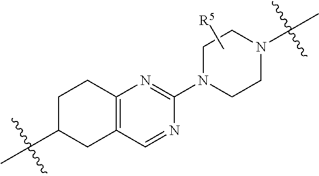

Embodiment II-54. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

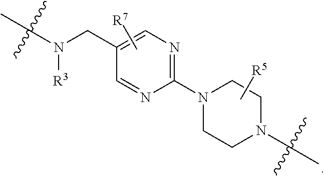

Embodiment II-55. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

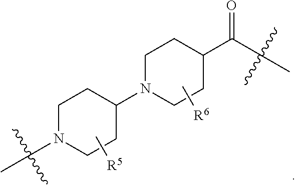

Embodiment II-56. The compound of any one of Embodiments II-1 to II-6, II-8, II-10 to II-11, II-13 to II-18, II-20, II-22 to II-23, II-25 to II-30, II-32, II-34 to II-35, and II-37 to II-45, wherein A¹ is

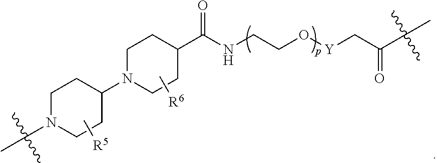

Embodiment II-57. The compound of any one of Embodiments II-1 to II-8, II-15 to II-20, II-27 to II-32, and II-39 to II-45, wherein A² is absent.

Embodiment II-58. The compound of any one of Embodiments II-1 to II-6, II-9 to II-18, II-21 to II-30, and II-33 to II-45, wherein A² is

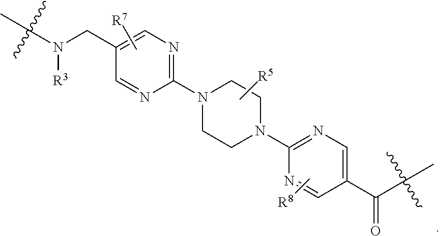

Embodiment II-59. The compound of any one of Embodiments II-1 to II-6, II-9 to II-18, II-21 to II-30, and II-33 to II-45, wherein A² is

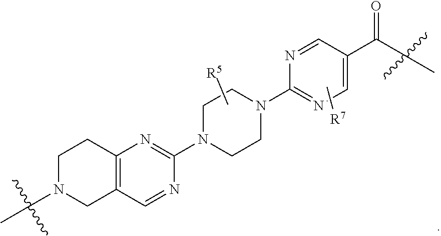

Embodiment II-60. The compound of any one of Embodiments II-1 to II-6, II-9 to II-18, II-21 to II-30, and II-33 to II-45, wherein A² is

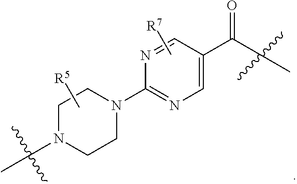

Embodiment II-61. The compound of any one of Embodiments II-1 to II-6, II-9 to II-18, II-21 to II-30, and II-33 to II-45, wherein A² is

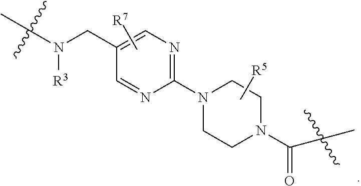

Embodiment II-62. The compound of any one of Embodiments II-1 to II-6, II-9 to II-18, II-21 to II-30, and II-33 to II-45, wherein A² is

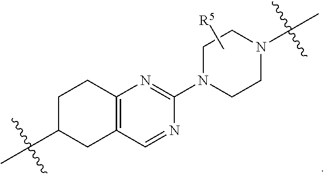

Embodiment II-63. The compound of any one of Embodiments II-1 to II-6, II-9 to II-18, II-21 to II-30, and II-33 to II-45, wherein A² is

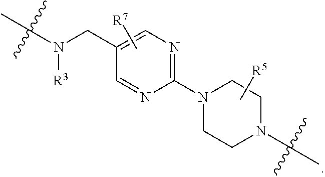

Embodiment II-64. The compound of any one of Embodiments II-1 to II-6, II-9 to II-18, II-21 to II-30, and II-33 to II-45, wherein A² is

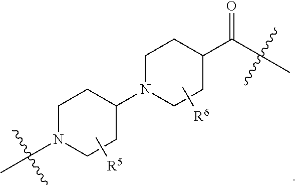

Embodiment II-65. The compound of any one of Embodiments II-1 to II-6, II-9 to II-18, II-21 to II-30, and II-33 to II-45, wherein A² is

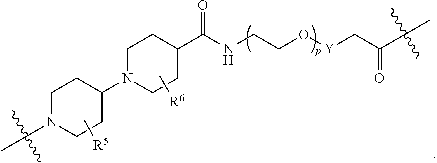

Embodiment II-66. The compound of any one of Embodiments II-1 to II-65, wherein B is

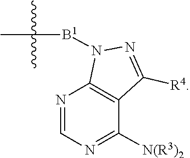

Embodiment II-67. The compound of any one of Embodiments II-1 to II-65, wherein B is

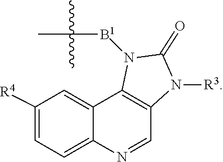

Embodiment II-68. The compound of any one of Embodiments II-1 to II-65, wherein B¹ is

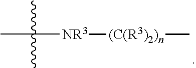

Embodiment II-69. The compound of any one of Embodiments II-1 to II-65, wherein B¹ is

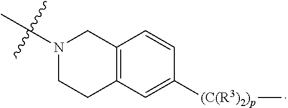

Embodiment II-70. The compound of any one of Embodiments II-1 to II-69, wherein R⁴ is 5-12 membered heteroaryl, optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl.

Embodiment II-71. The compound of any one of Embodiments II-1 to II-70, or a pharmaceutically acceptable salt or tautomer thereof, wherein compound has the following formula:

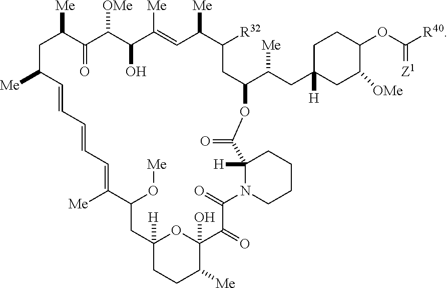

Embodiment II-72. A compound selected from the group consisting of:

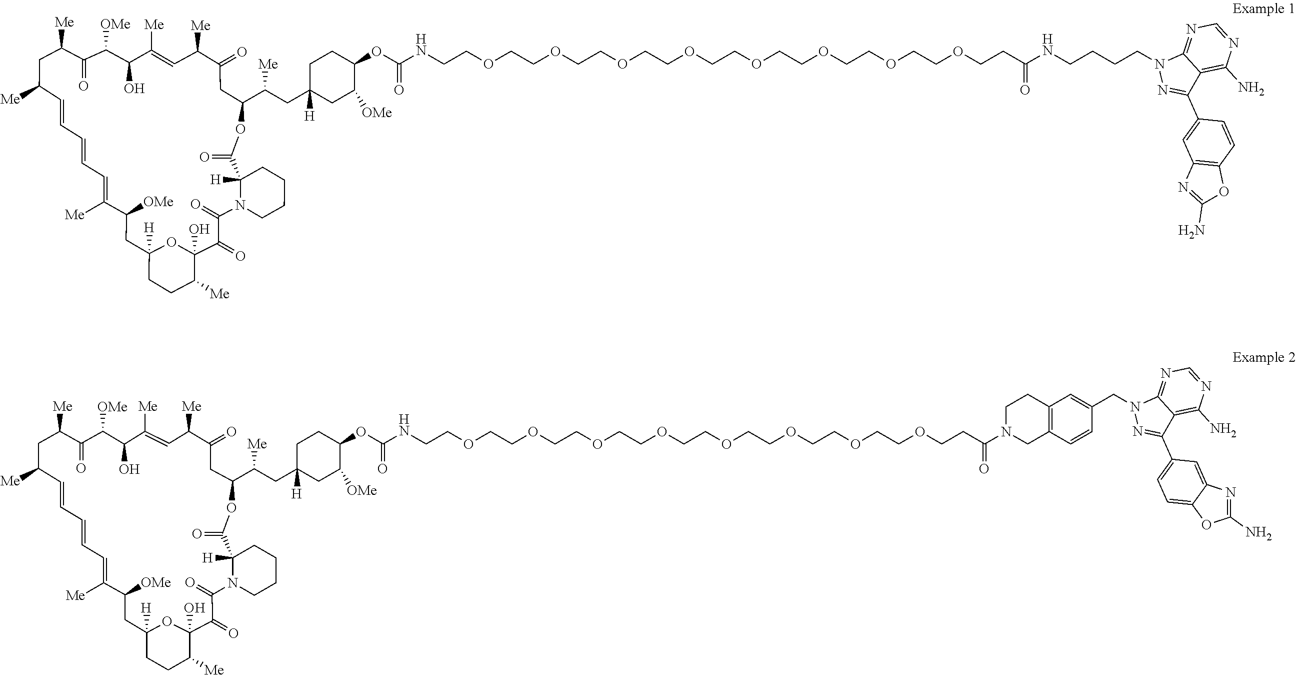

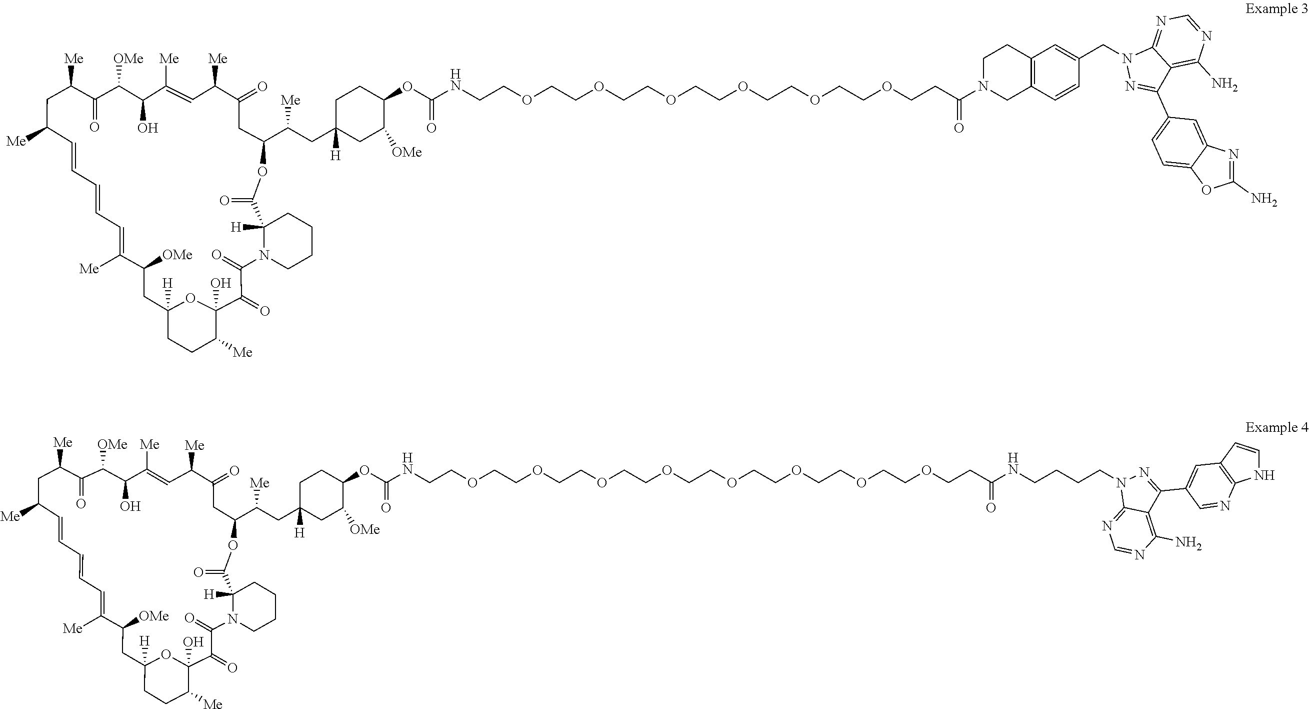

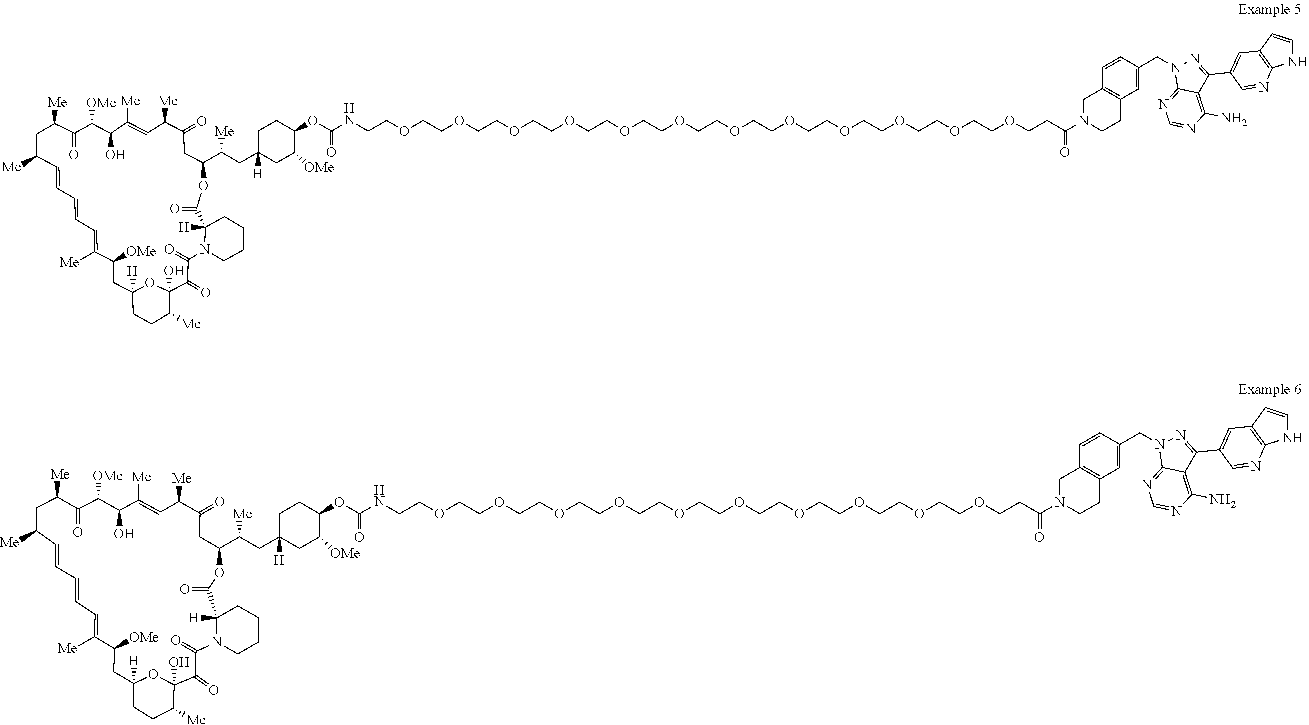

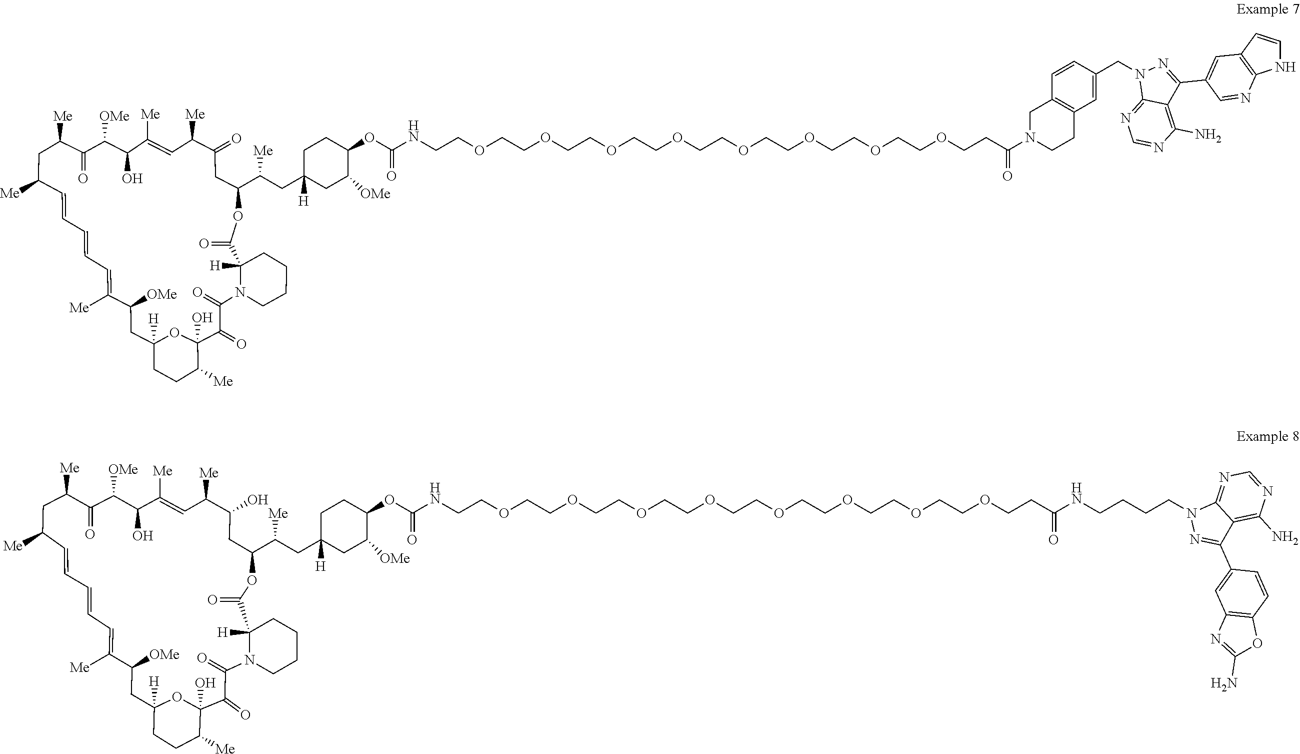

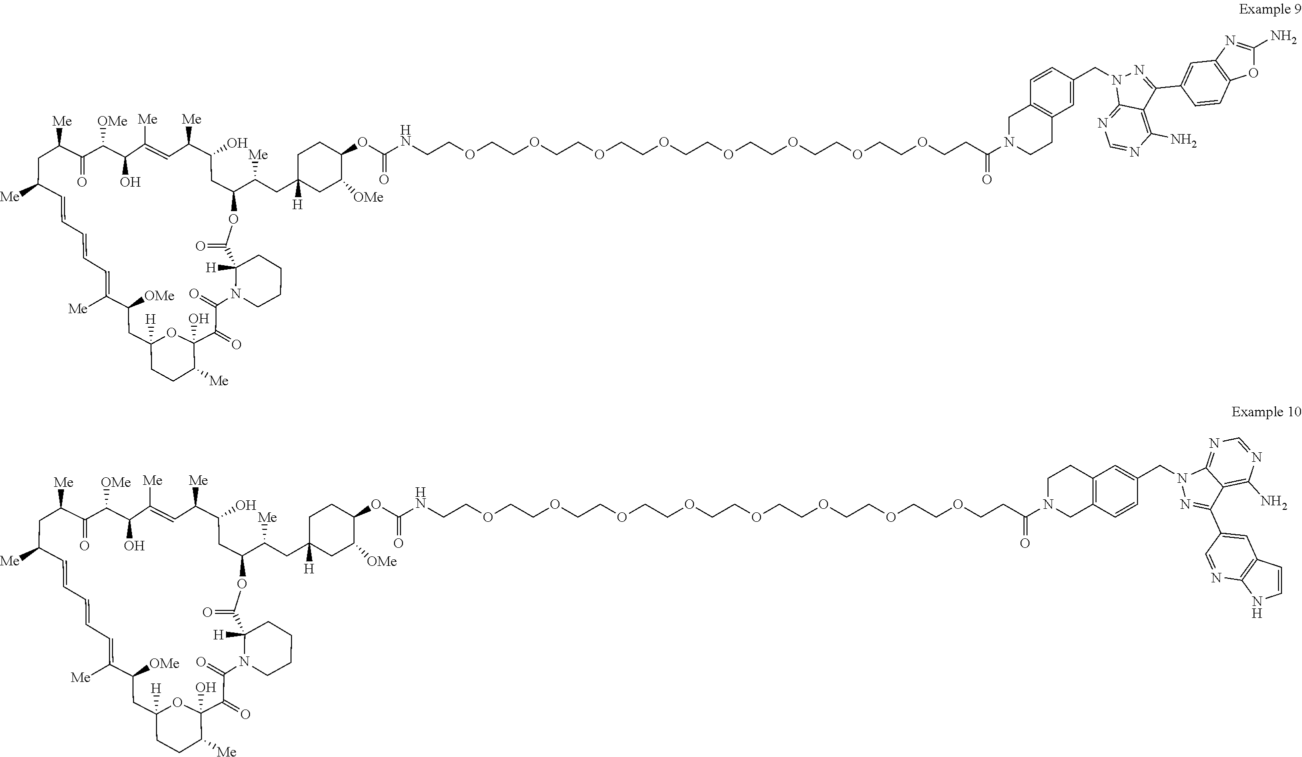

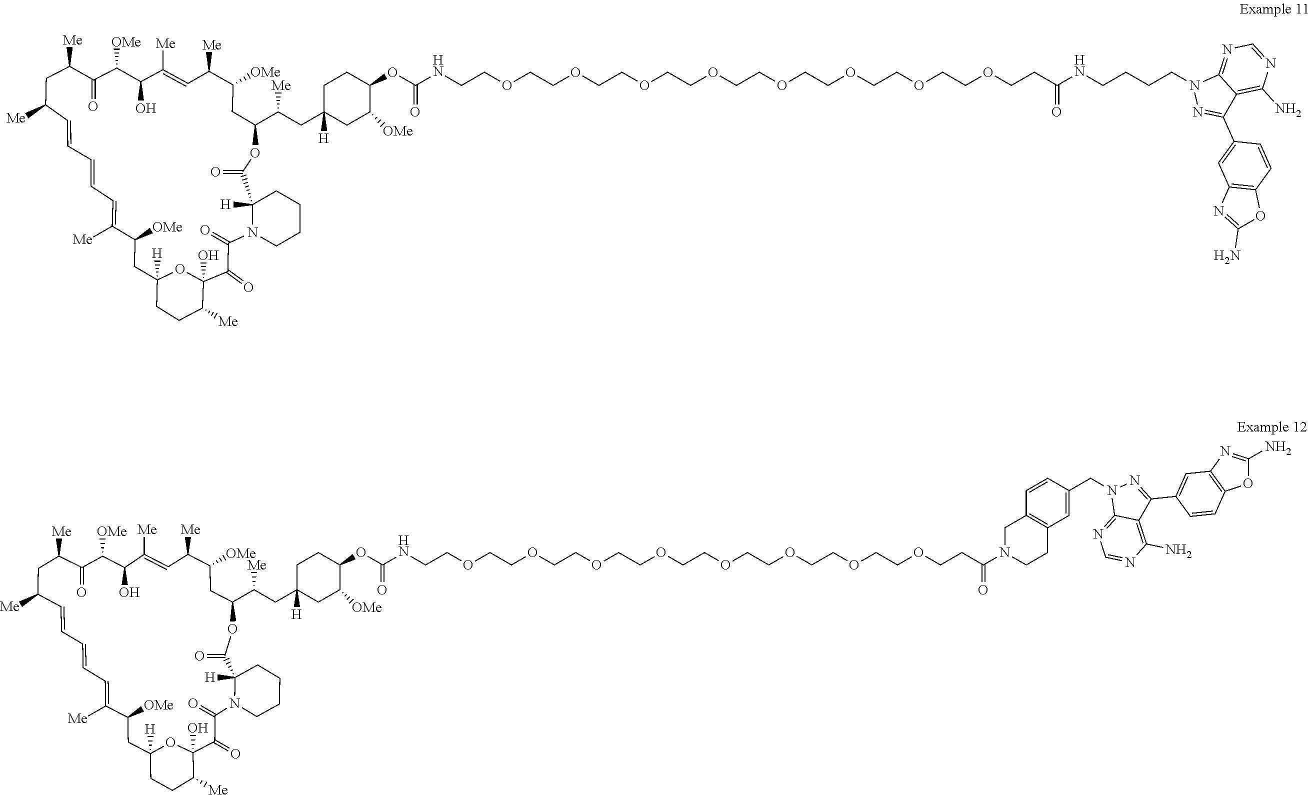

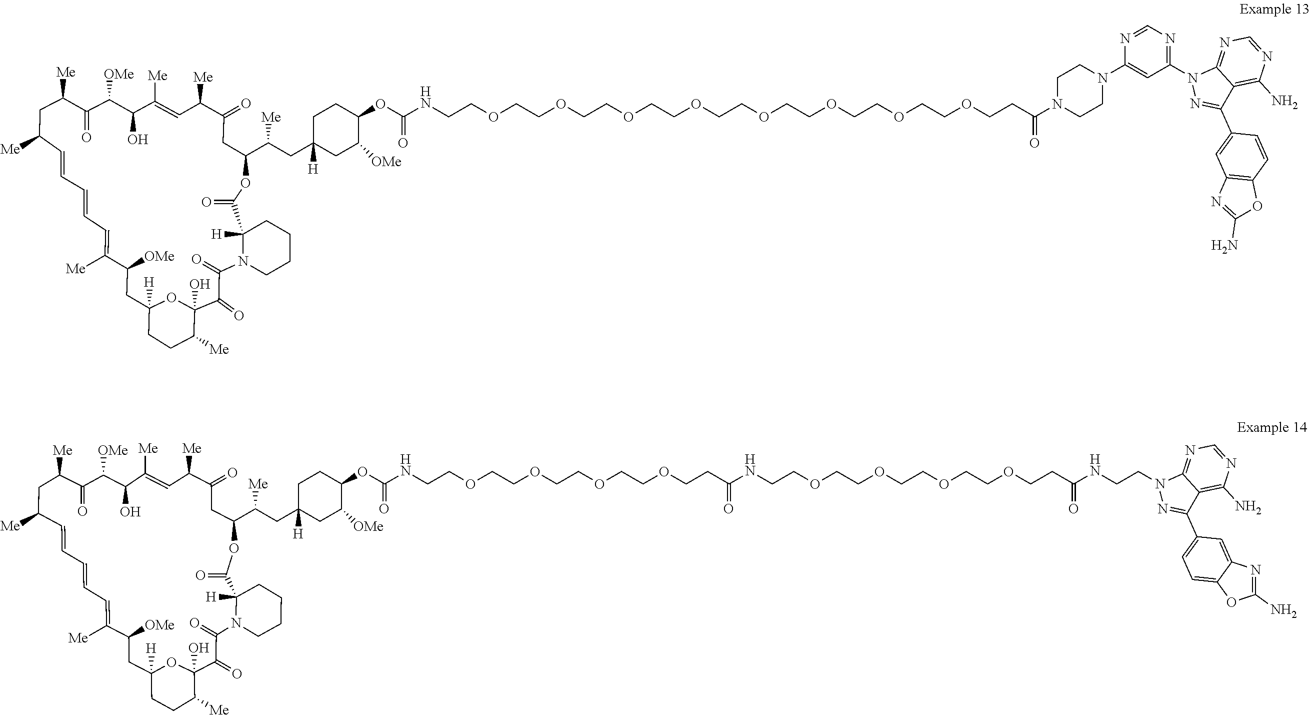

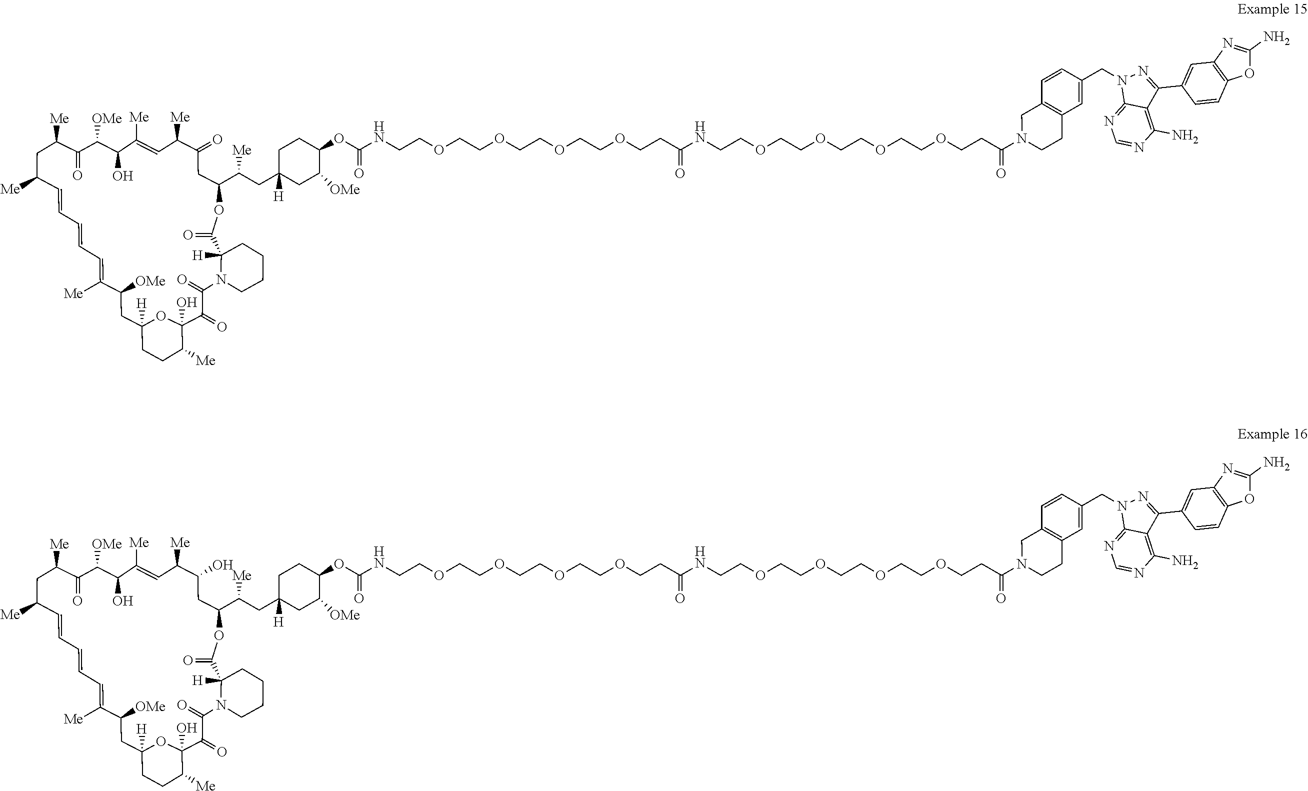

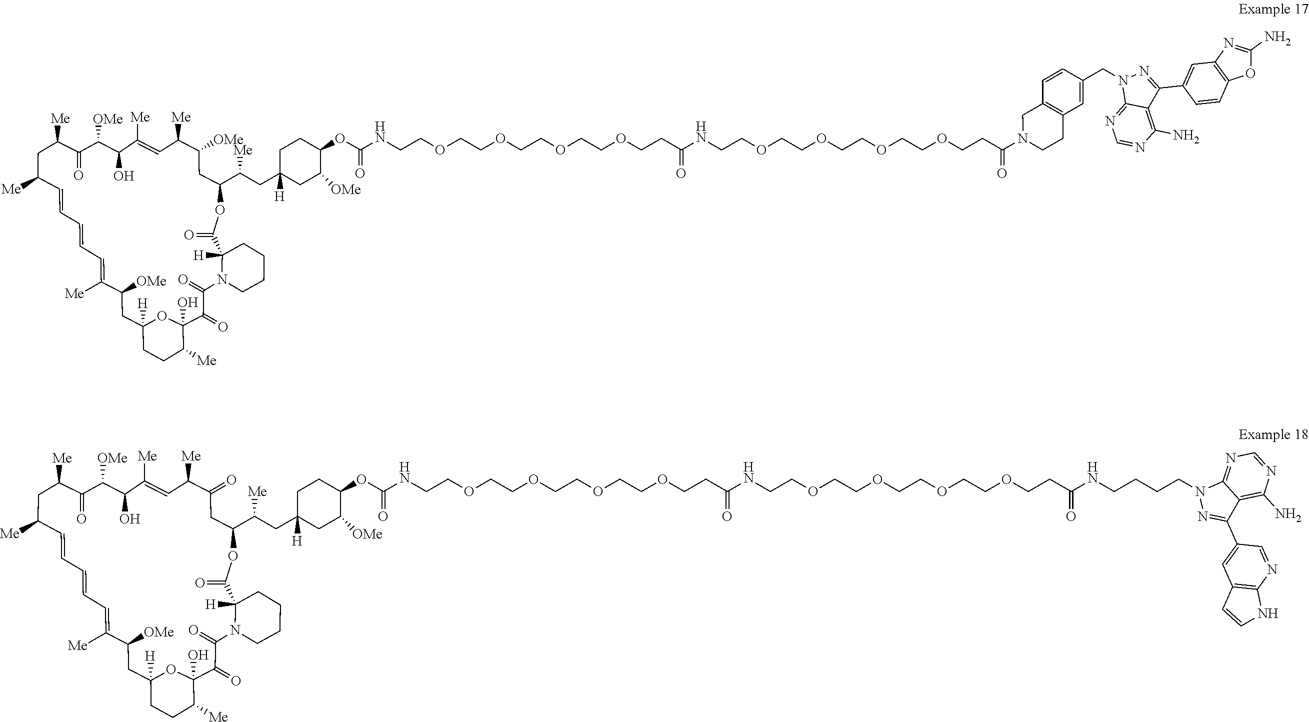

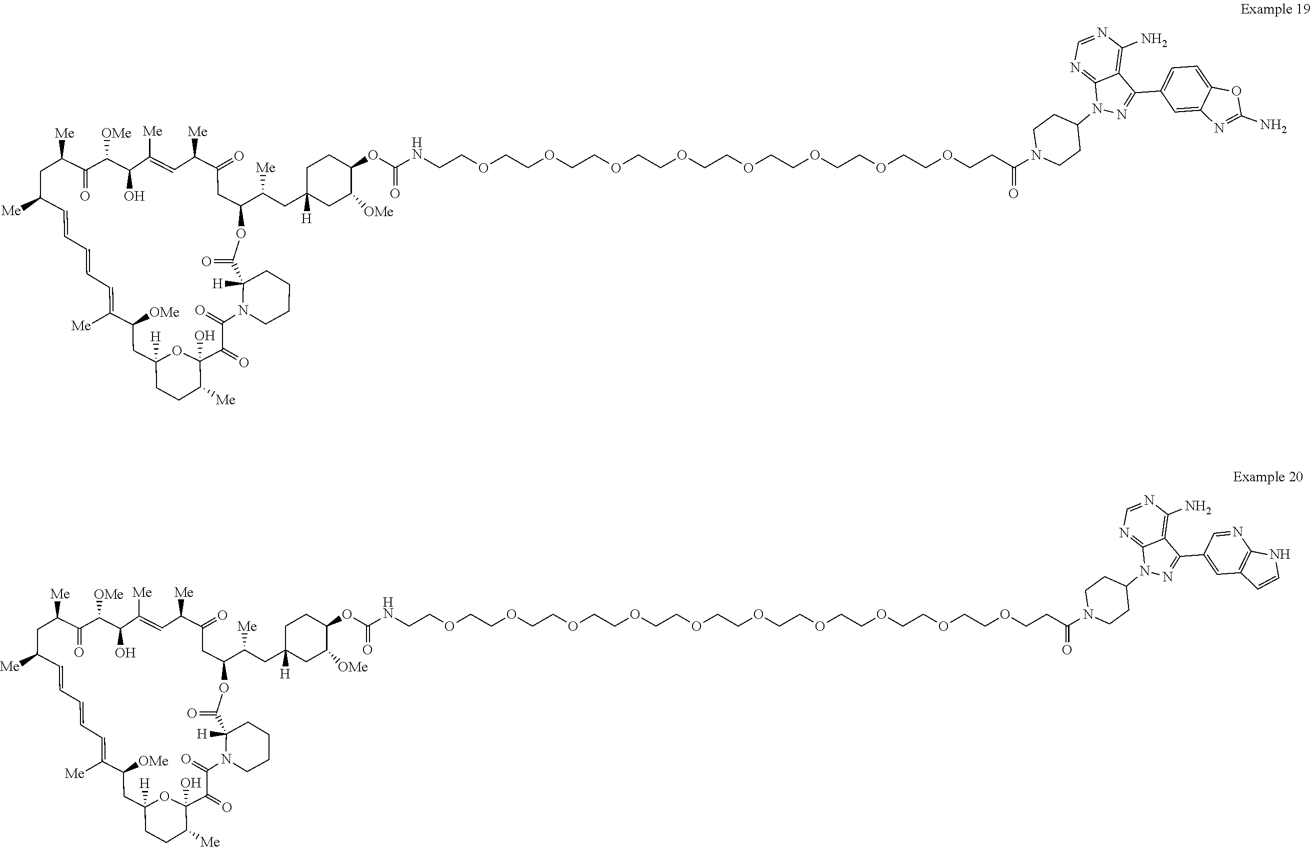

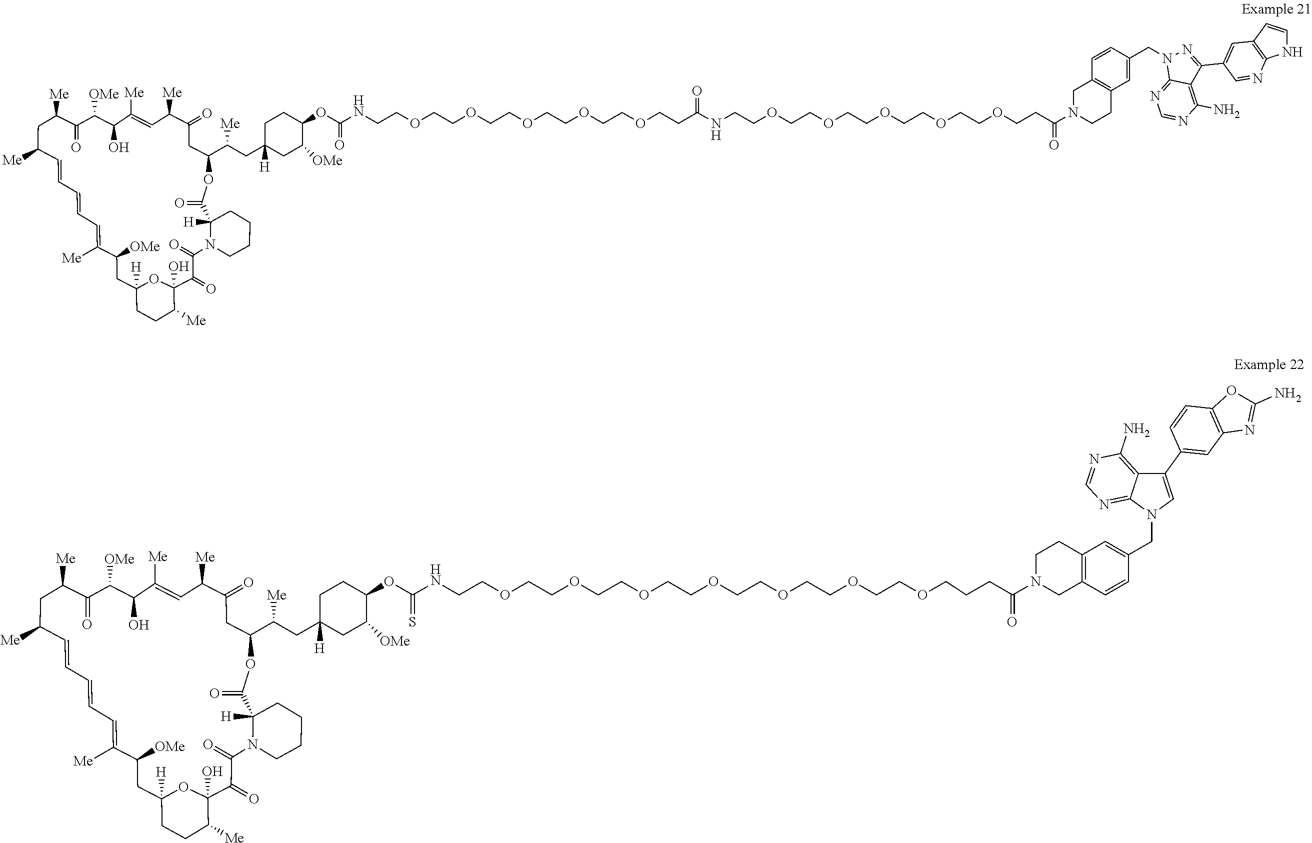

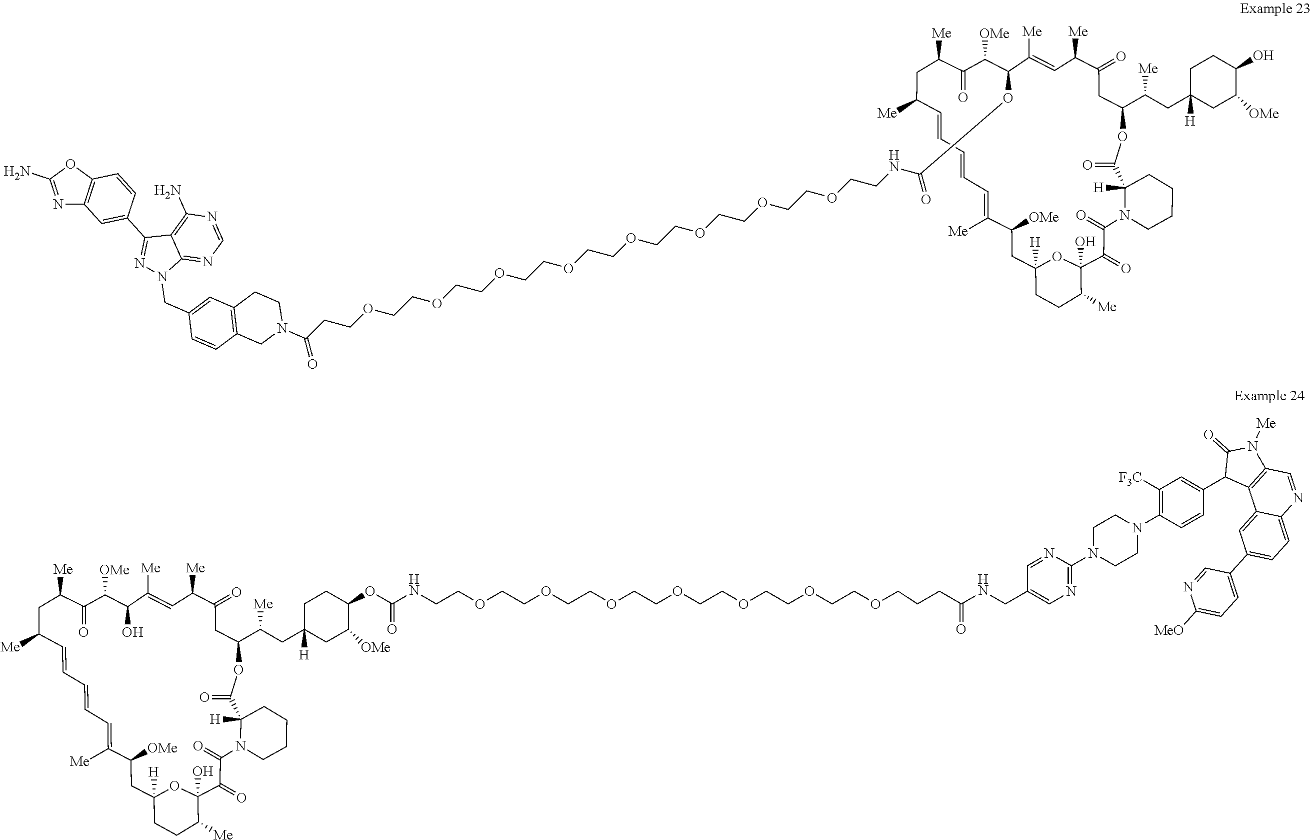

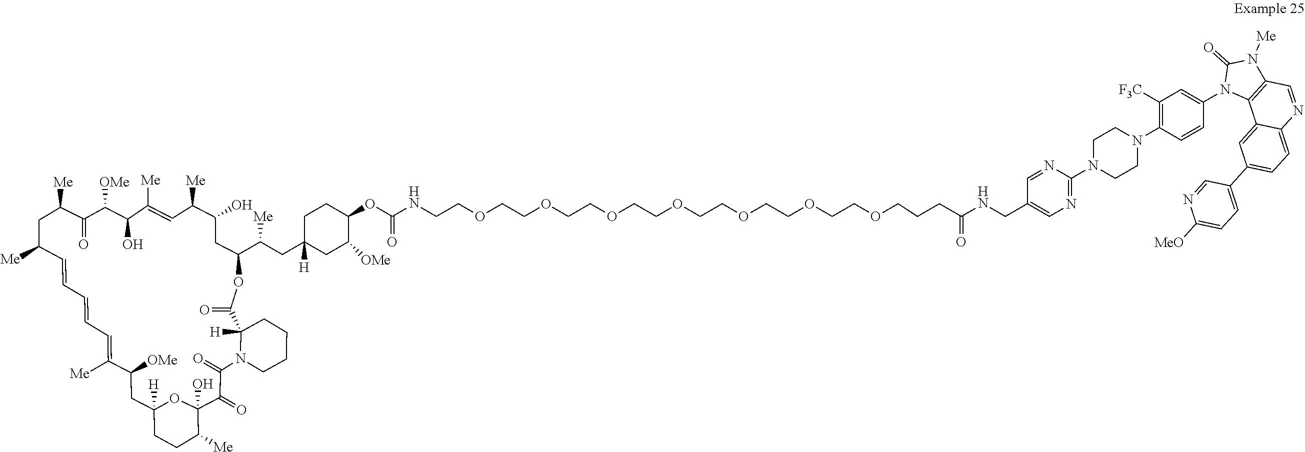 [SMILES: COc1ccc(-c2ccc3ncc4c(c3c2)n(-c2ccc(N3CCN(c5ncc(CNC(=O)CCCOCCOCCOCCOCCOCCOCCOCCNC(=O)O[C@@H]6CC[C@@H](C[C@@H](C)[C@@H]7C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)cn5)CC3)c(C(F)(F)F)c2)c(=O)n4C)cn1]

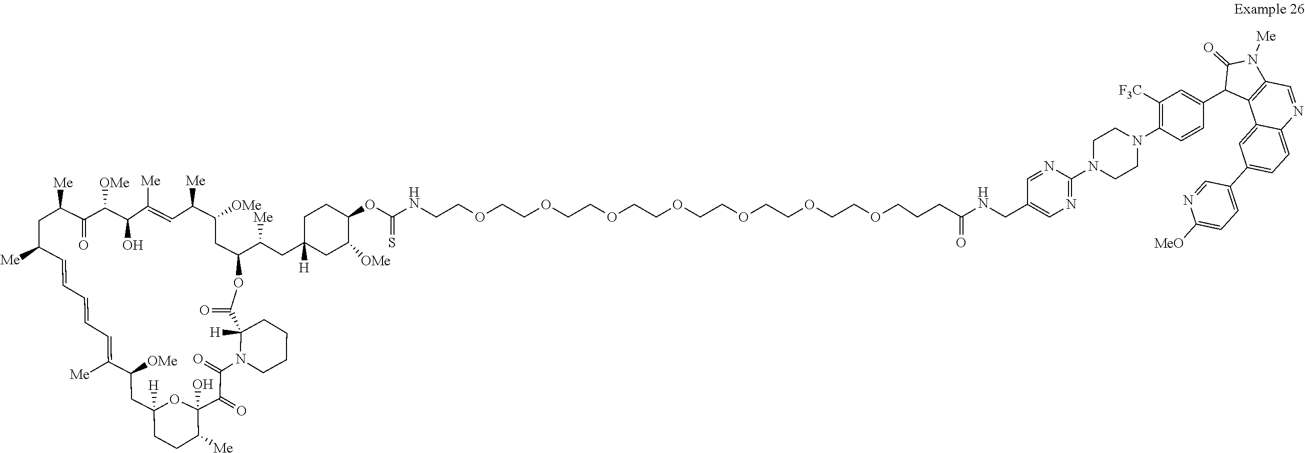 [SMILES: COc1ccc(-c2ccc3ncc4c(c3c2)C(c2ccc(N3CCN(c5ncc(CNC(=O)CCCOCCOCCOCCOCCOCCOCCOCCNC(=S)O[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)cn5)CC3)c(C(F)(F)F)c2)C(=O)N4C)cn1]

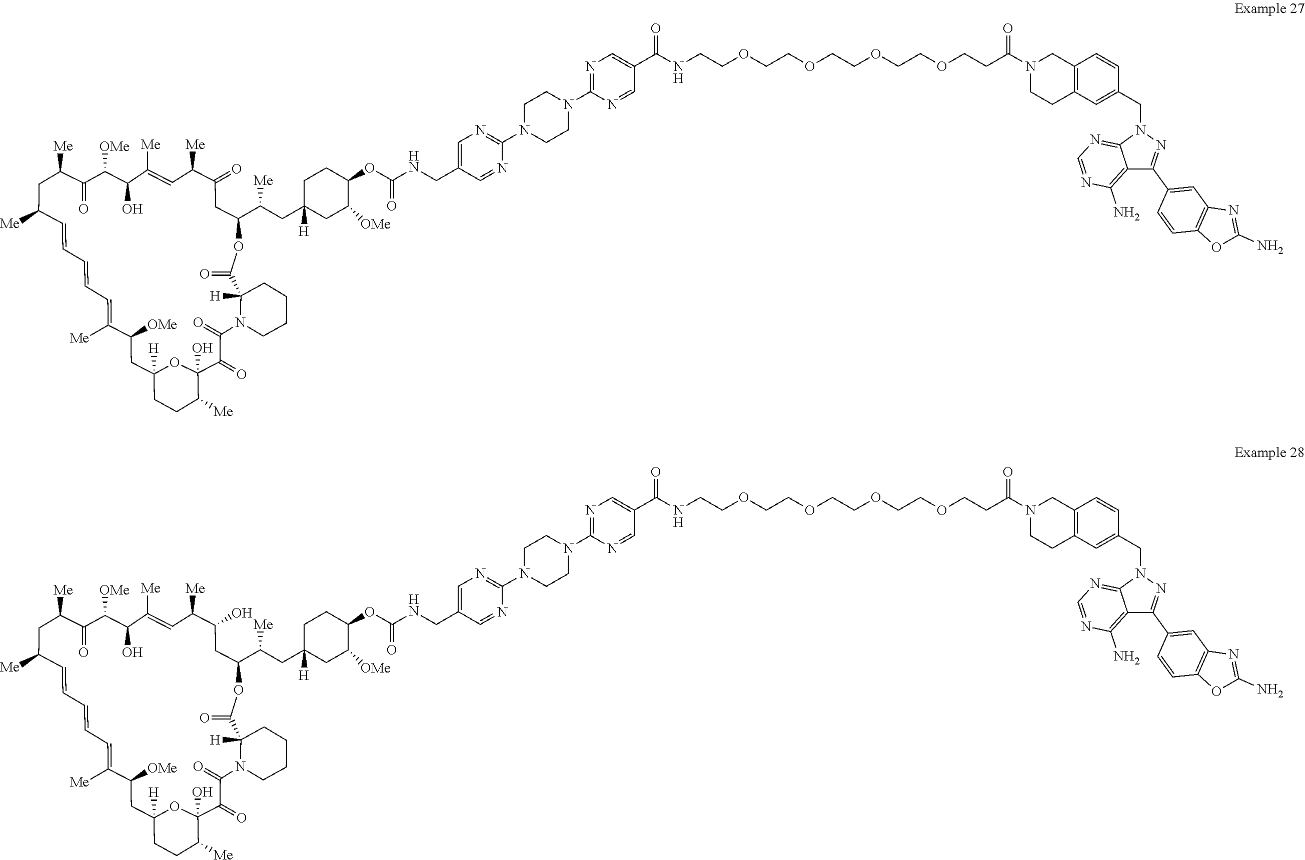

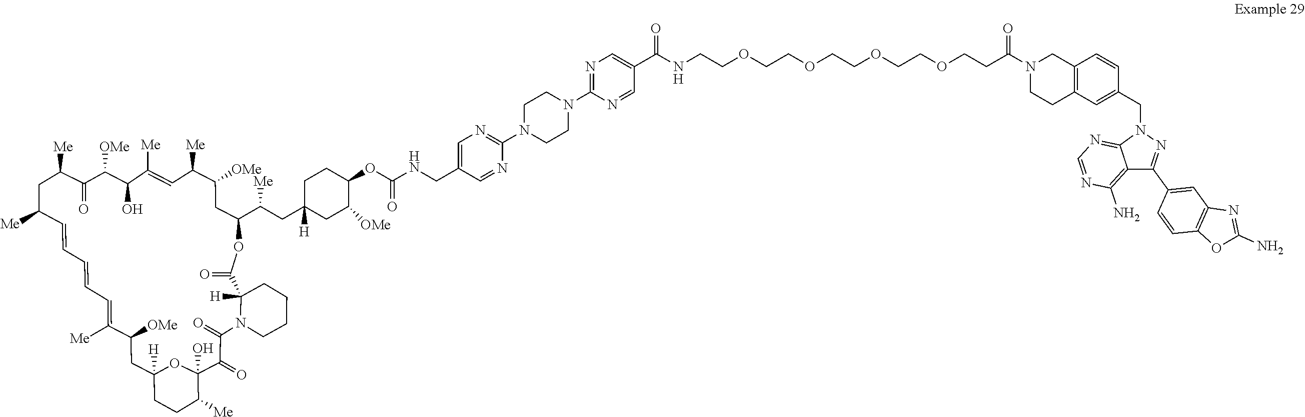 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

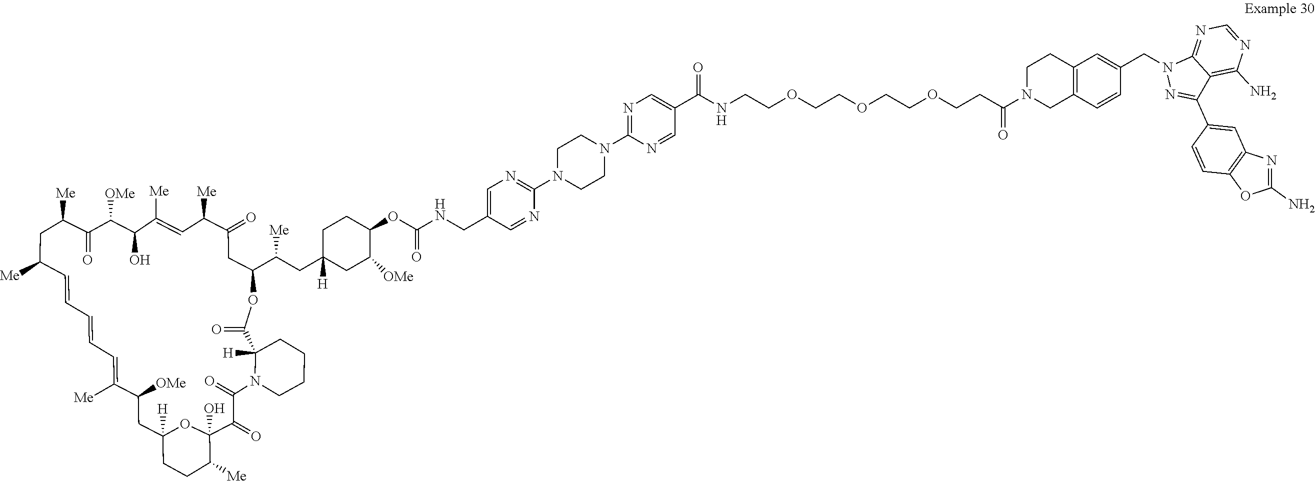 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

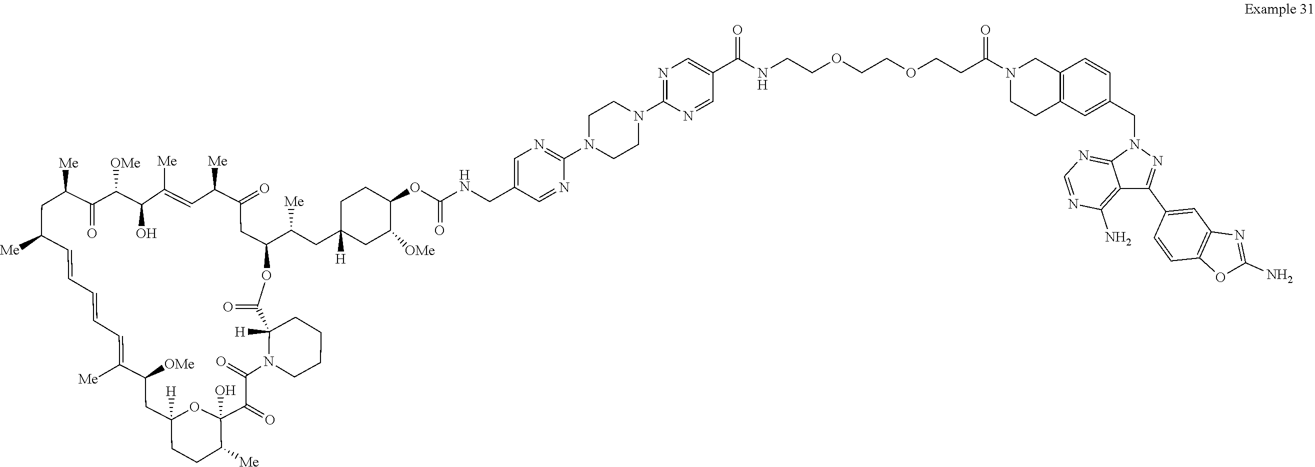 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

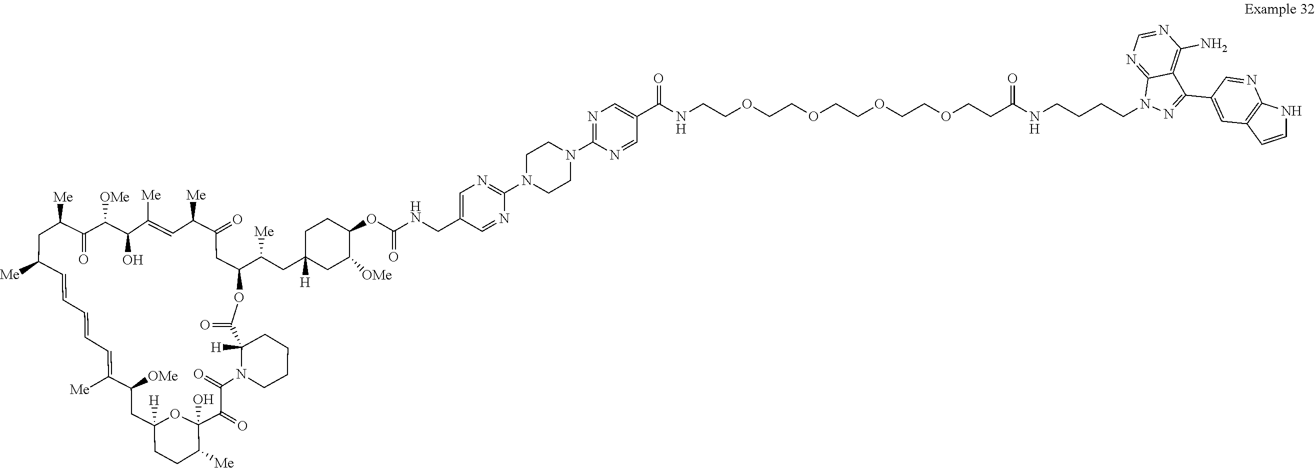 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

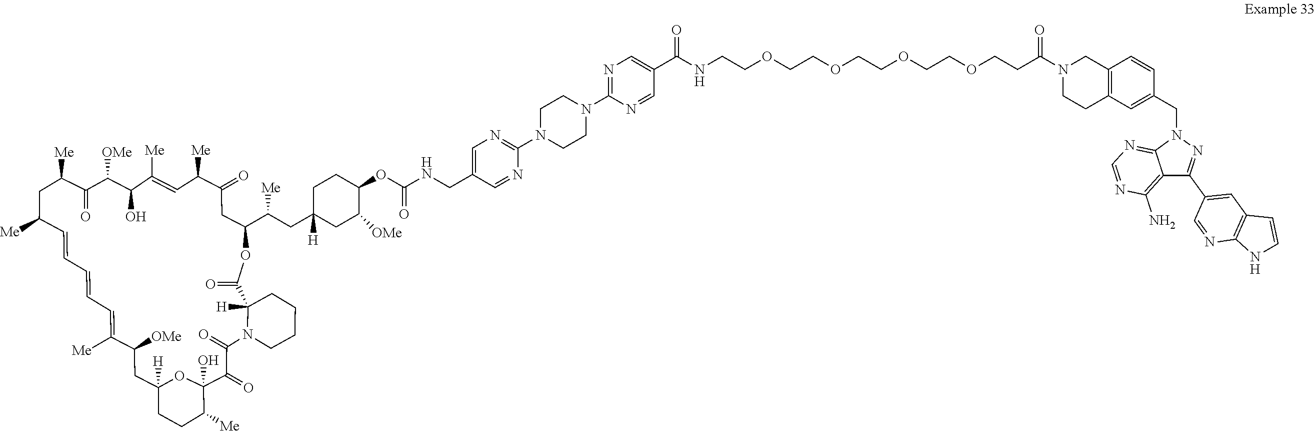 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

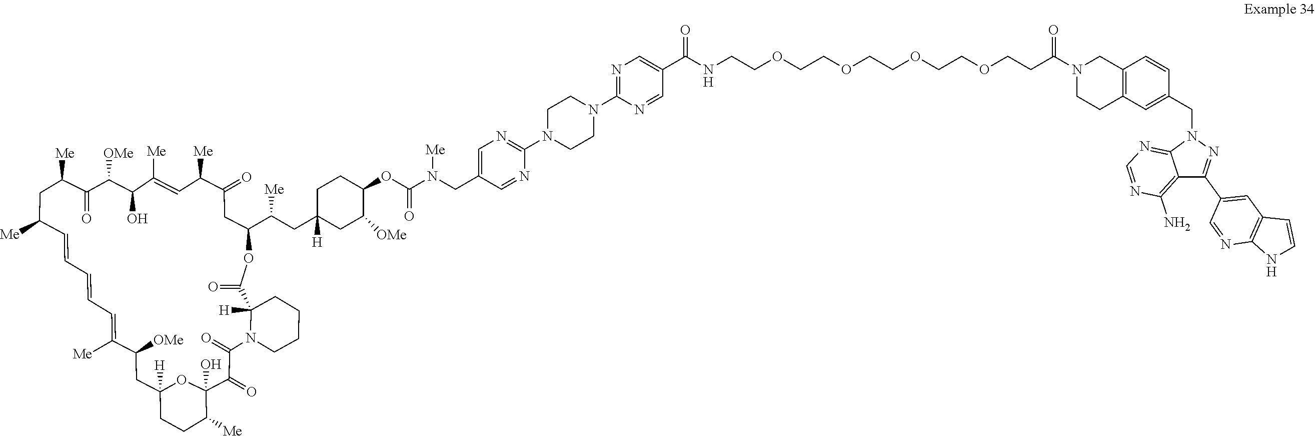 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)Cc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

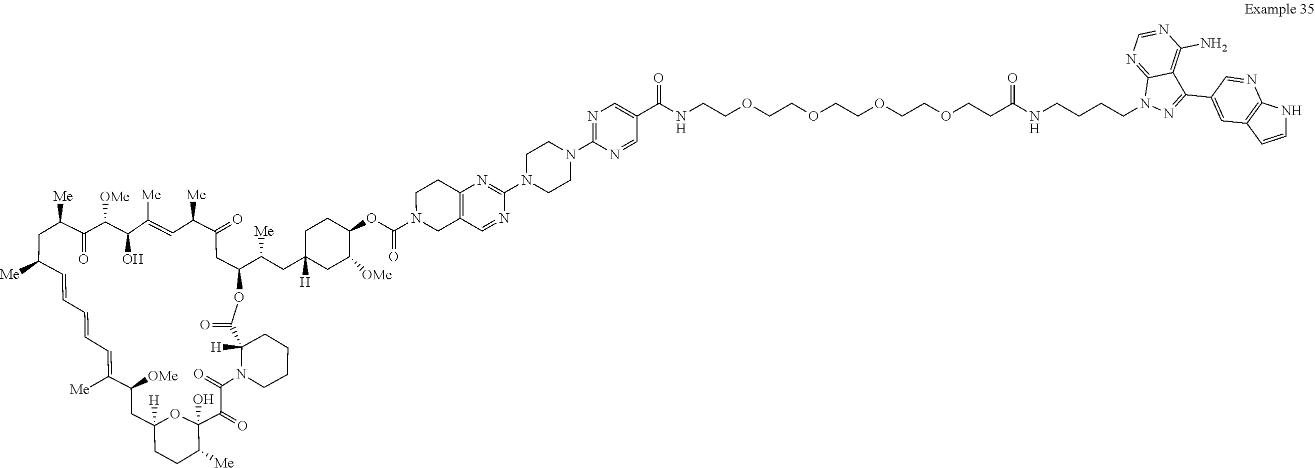 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)NCCCCn7nc(-c8cnc9[nH]ccc9c8)c8c(N)ncnc87)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

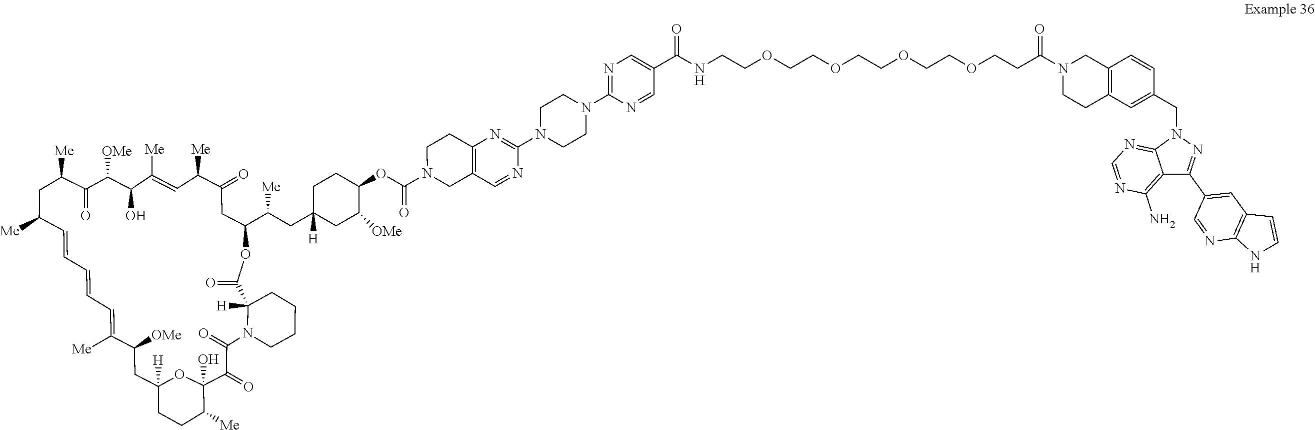 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

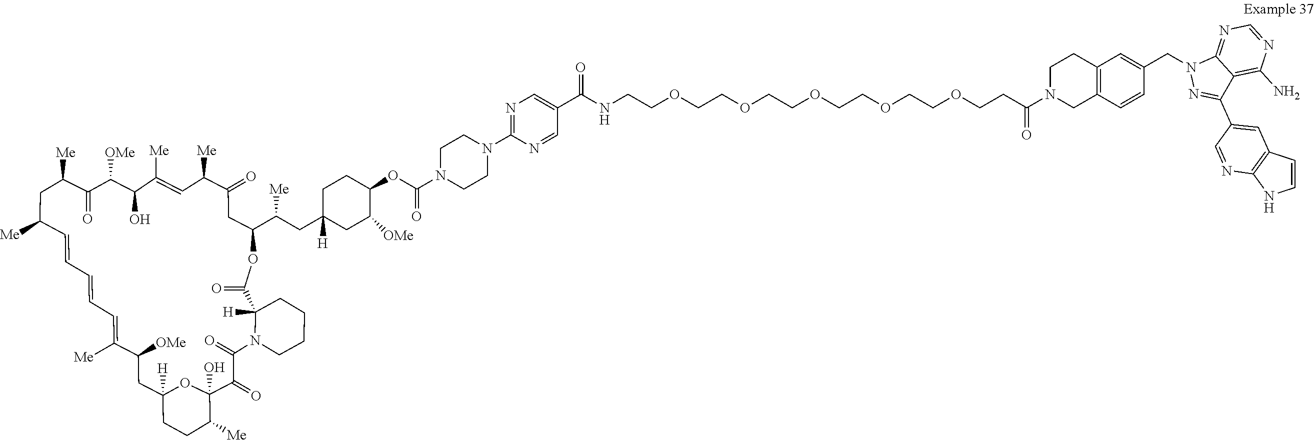 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(c4ncc(C(=O)NCCOCCOCCOCCOCCOCCC(=O)N5CCc6cc(Cn7nc(-c8cnc9[nH]ccc9c8)c8c(N)ncnc87)ccc6C5)cn4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C]

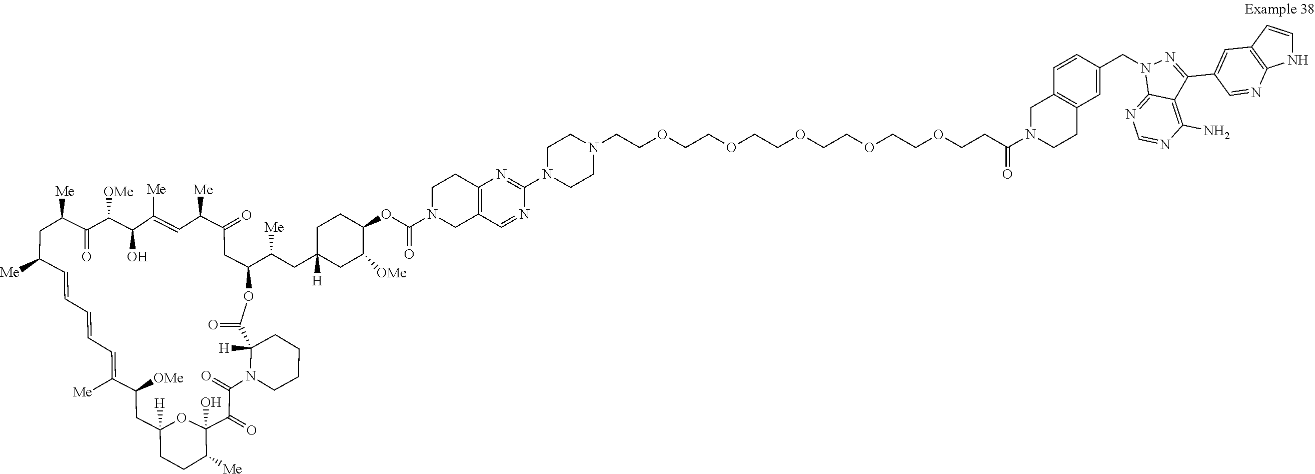 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCOCCOCCOCCOCCC(=O)N6CCc7cc(Cn8nc(-c9cnc%10[nH]ccc%10c9)c9c(N)ncnc98)ccc7C6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

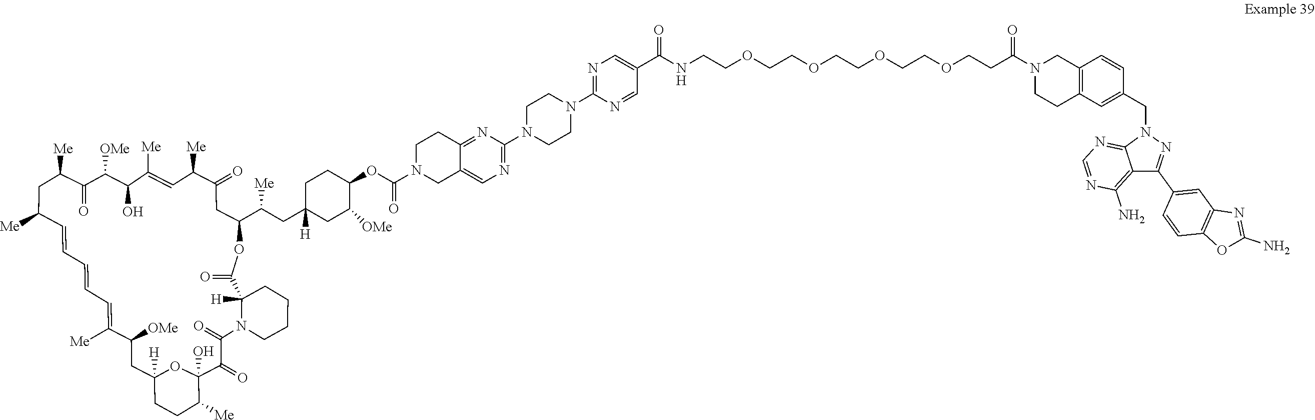 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

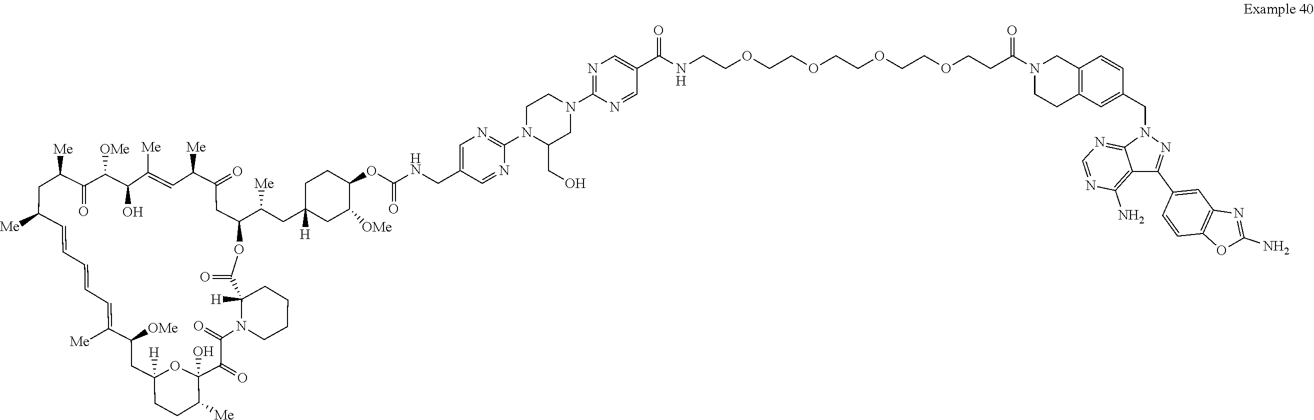 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

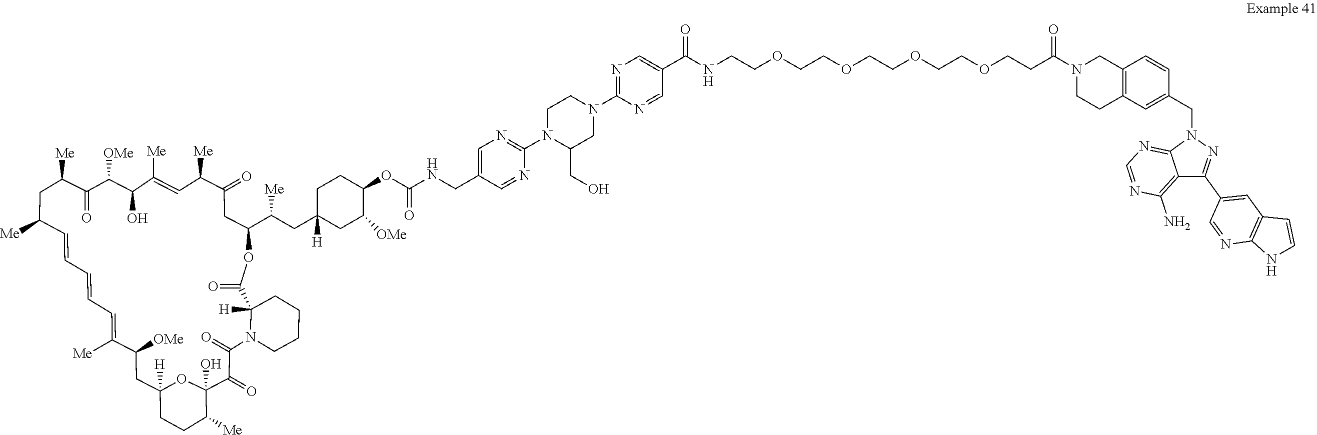 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

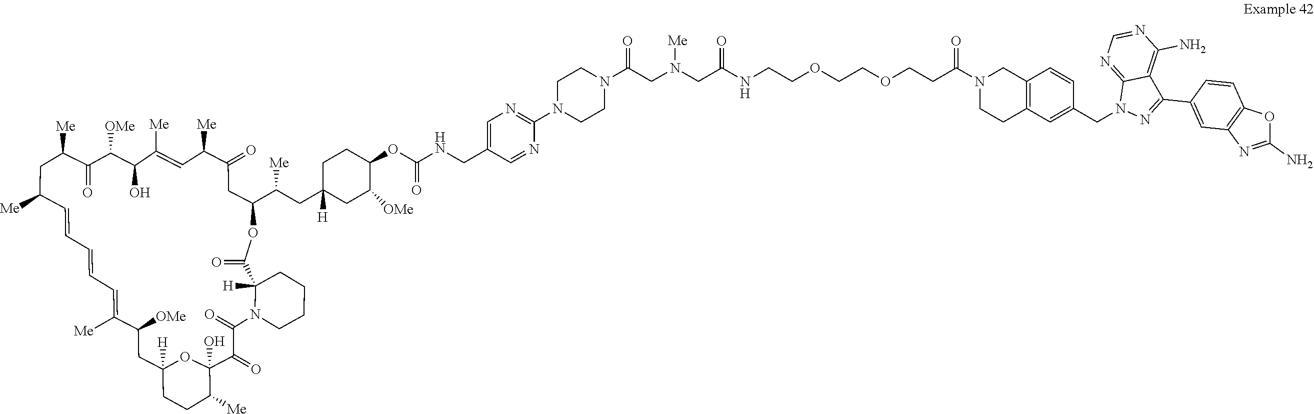 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CN(C)CC(=O)NCCOCCOCCC(=O)N5CCc6cc(Cn7nc(-c8ccc9oc(N)nc9c8)c8c(N)ncnc87)ccc6C5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

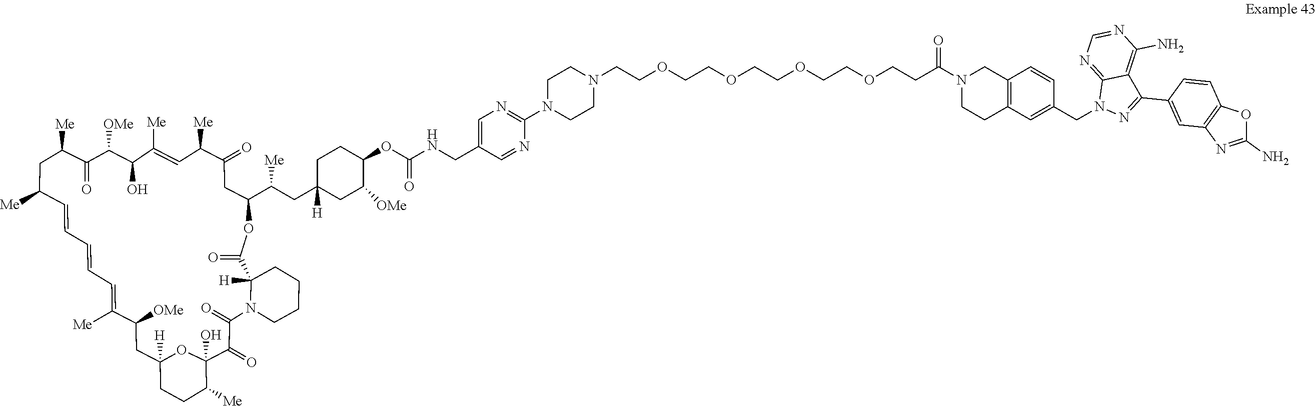 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)N5CCc6cc(Cn7nc(-c8ccc9oc(N)nc9c8)c8c(N)ncnc87)ccc6C5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

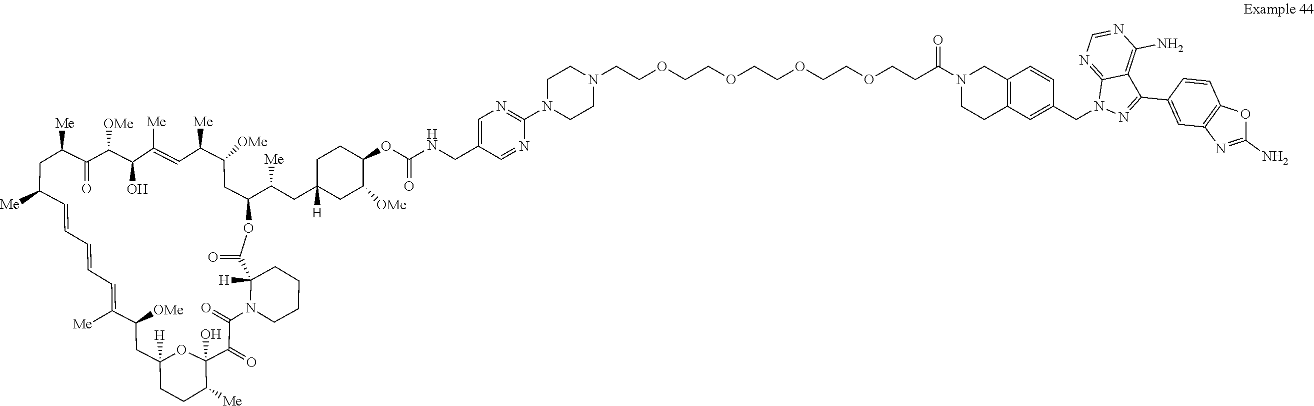 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)N5CCc6cc(Cn7nc(-c8ccc9oc(N)nc9c8)c8c(N)ncnc87)ccc6C5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

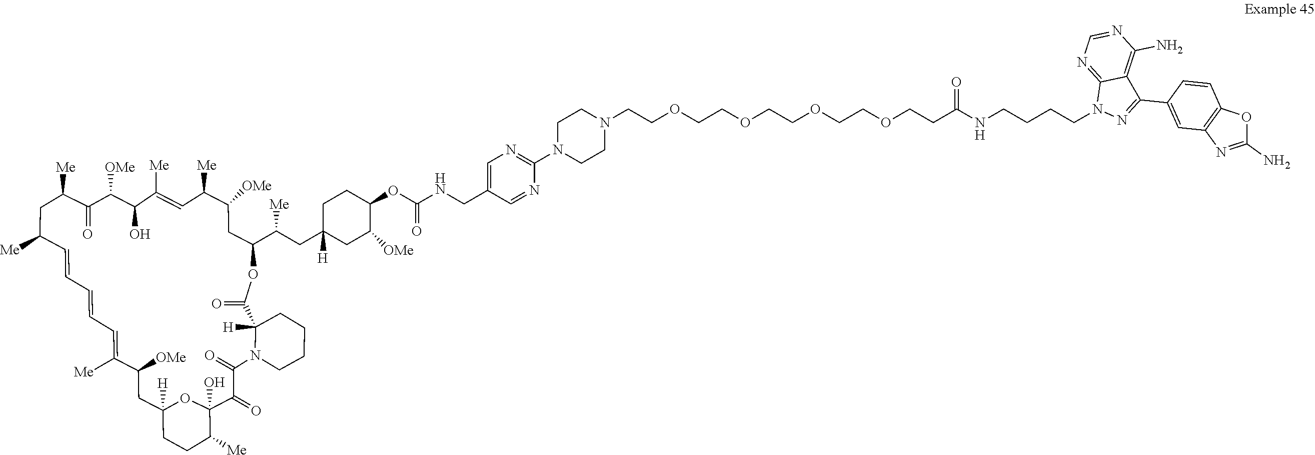 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)NCCCCn5nc(-c6ccc7oc(N)nc7c6)c6c(N)ncnc65)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

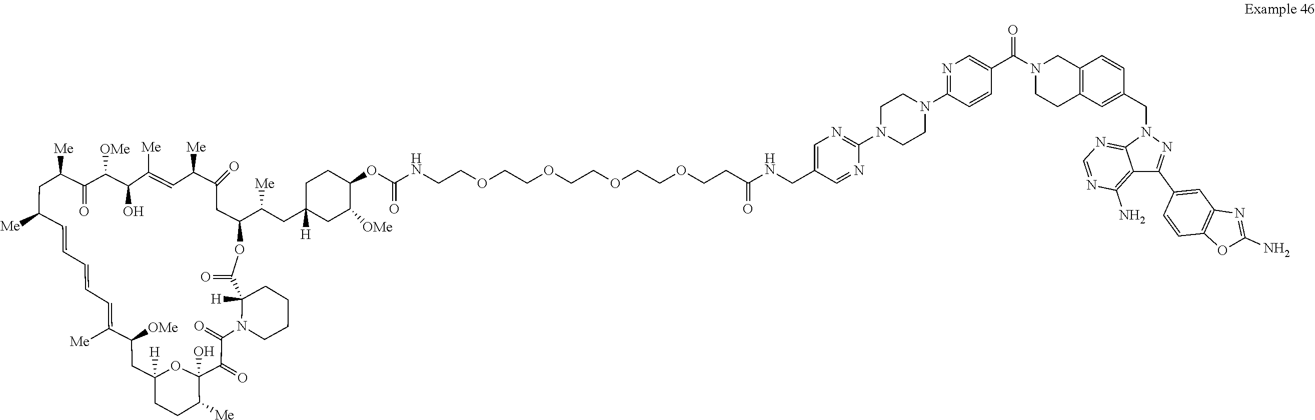 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ccc(C(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

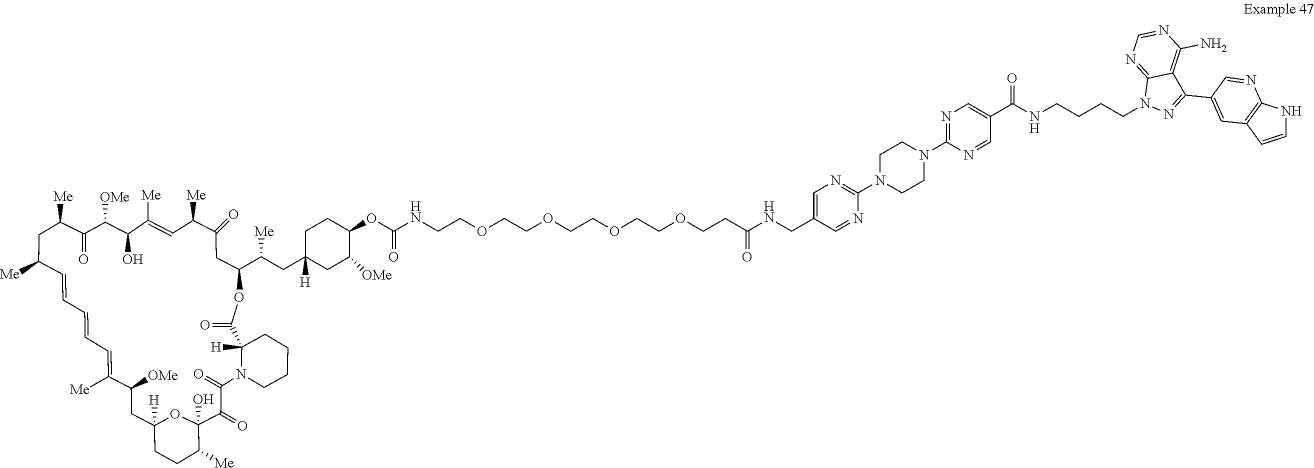 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

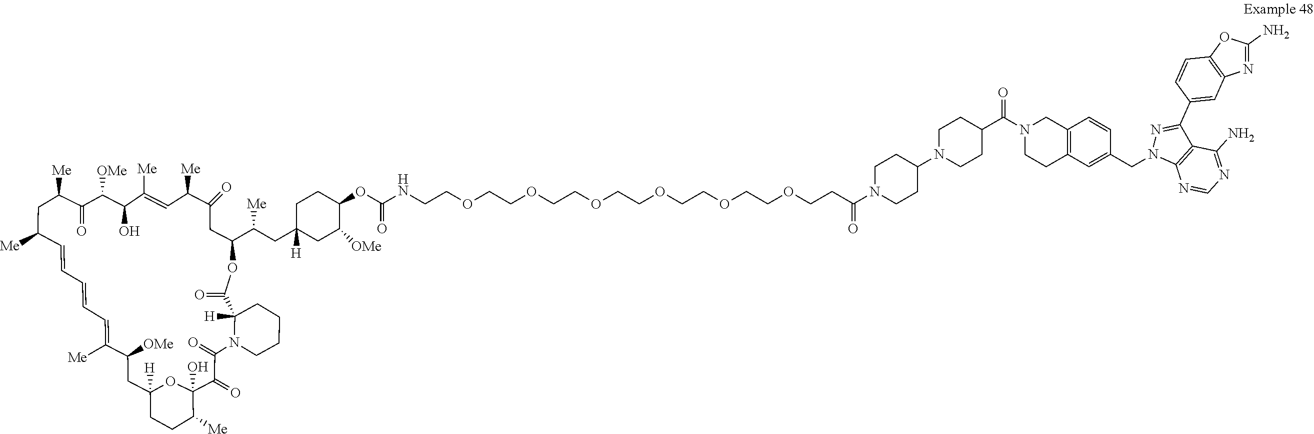 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCC(=O)N3CCC(N4CCC(C(=O)N5CCc6cc(Cn7nc(-c8ccc9oc(N)nc9c8)c8c(N)ncnc87)ccc6C5)CC4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C]

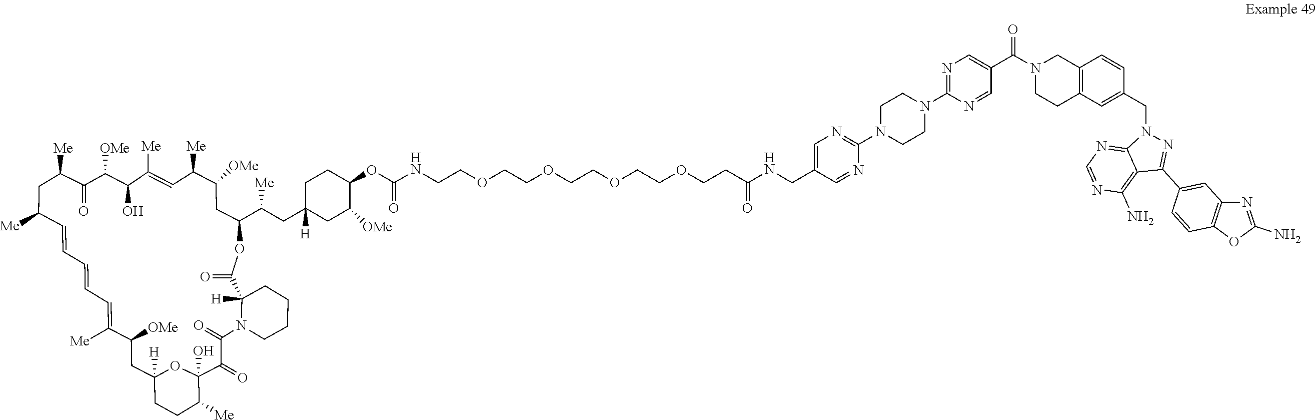 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

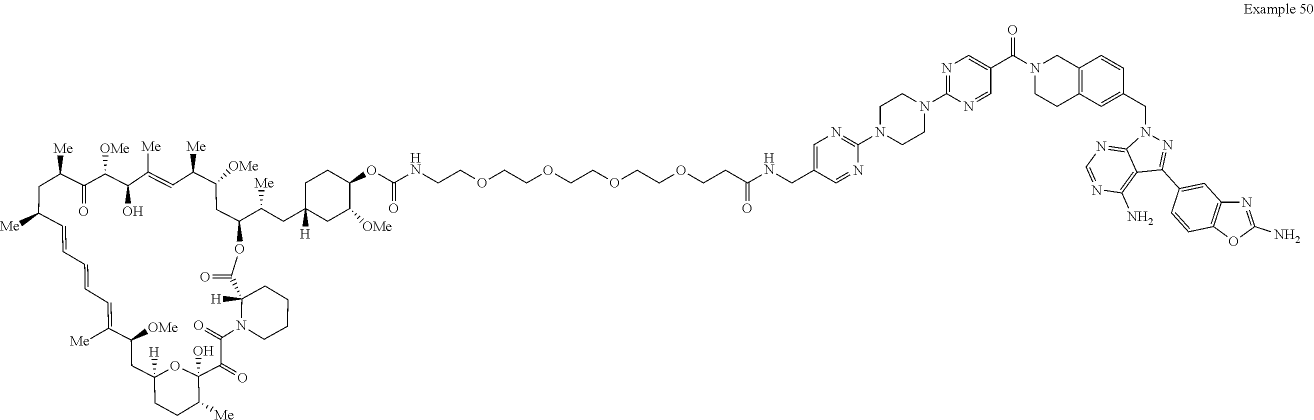 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

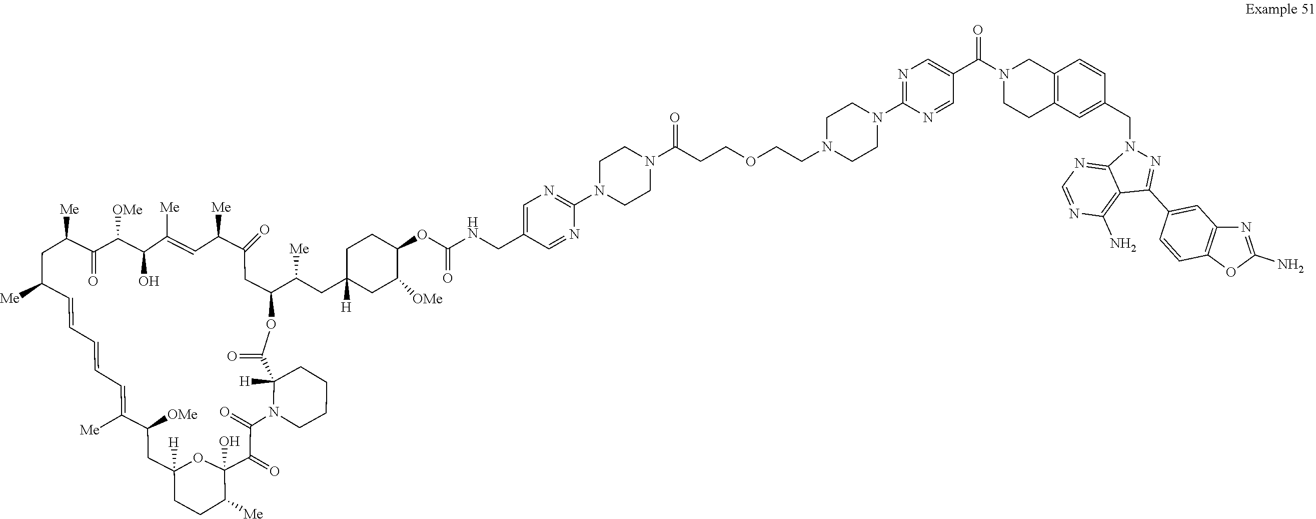 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

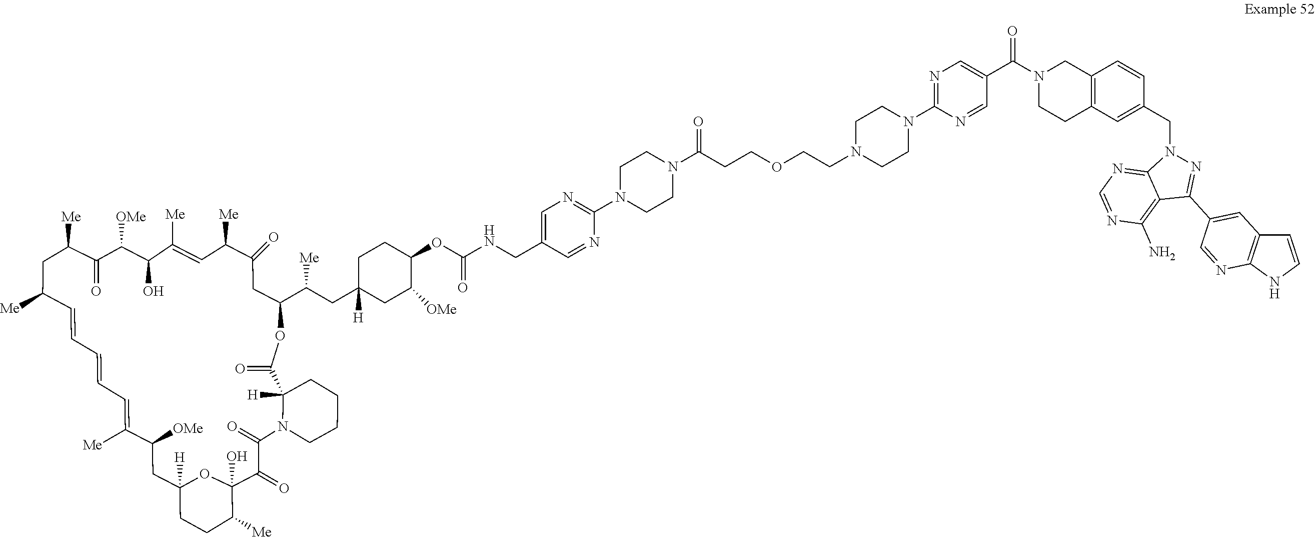 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

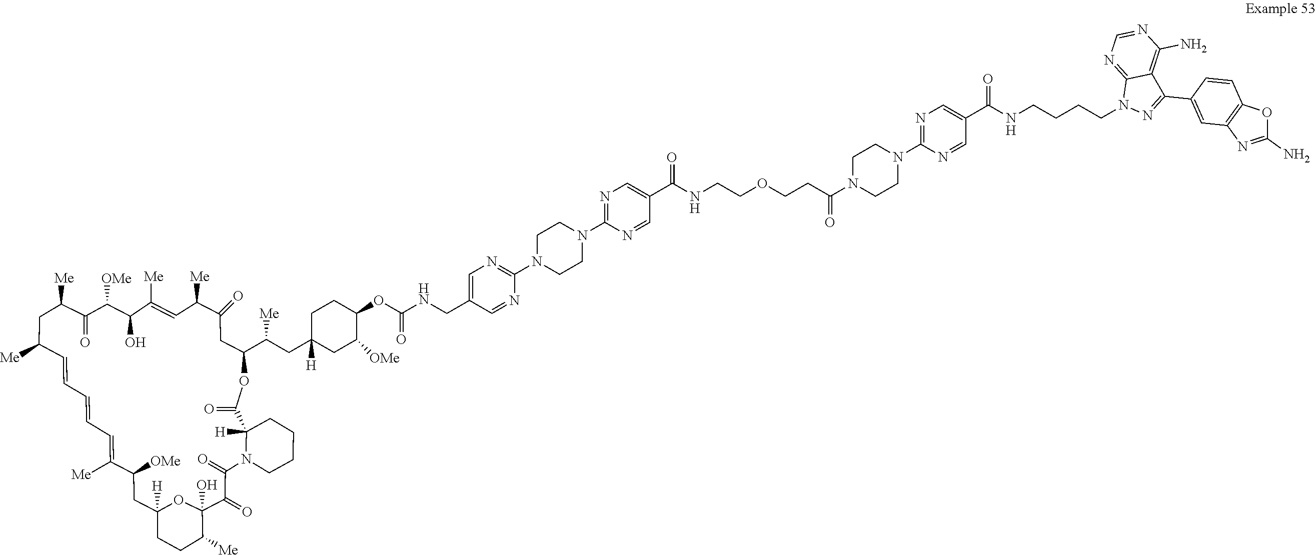 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCC(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

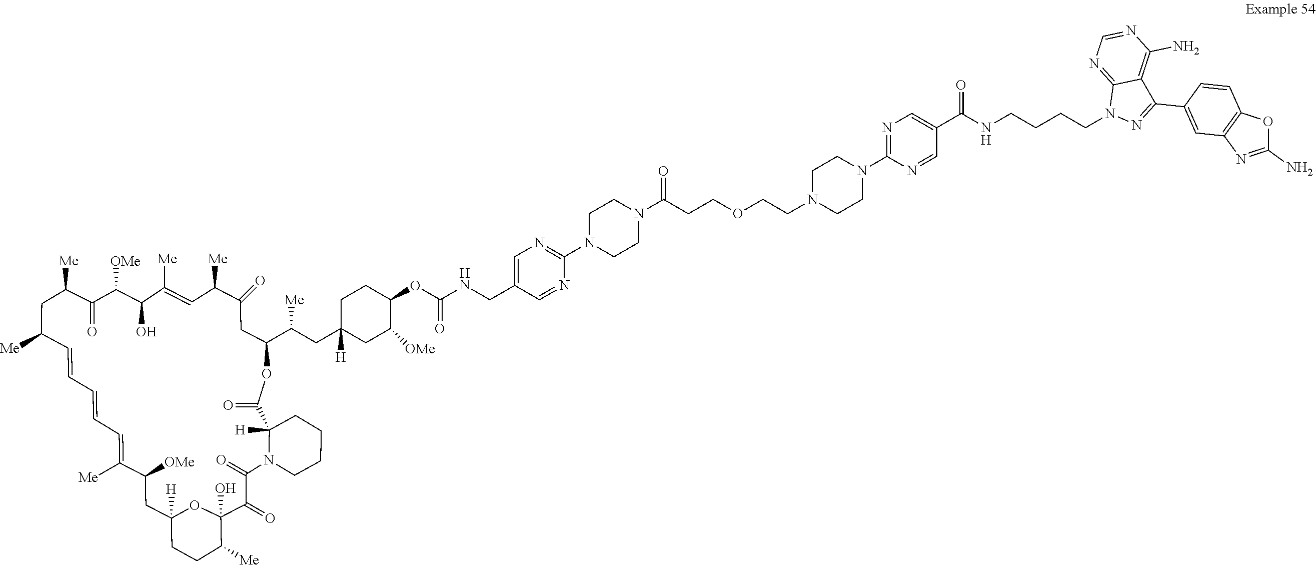 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

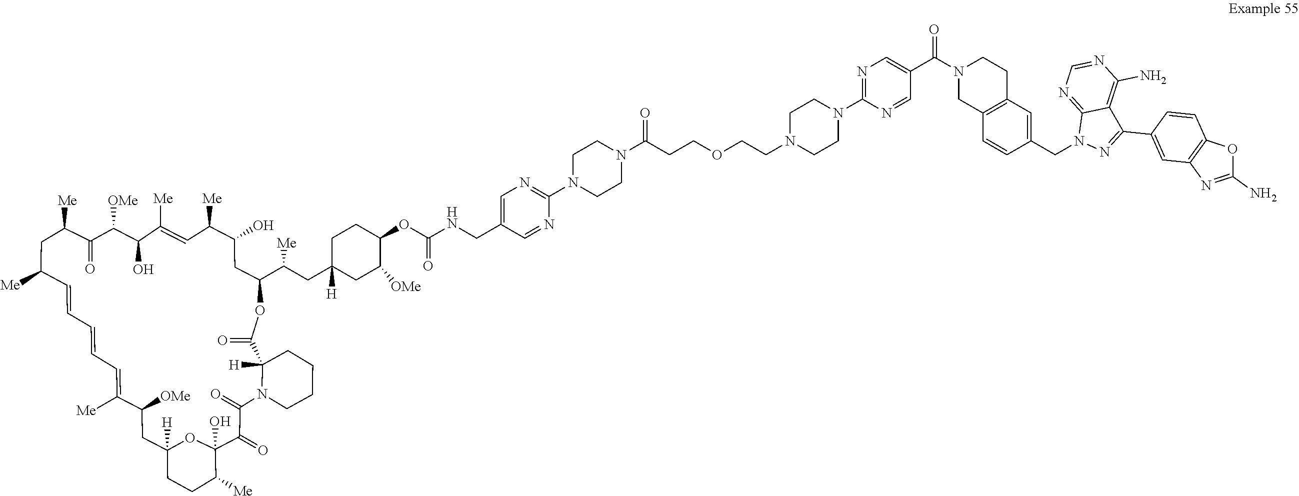 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

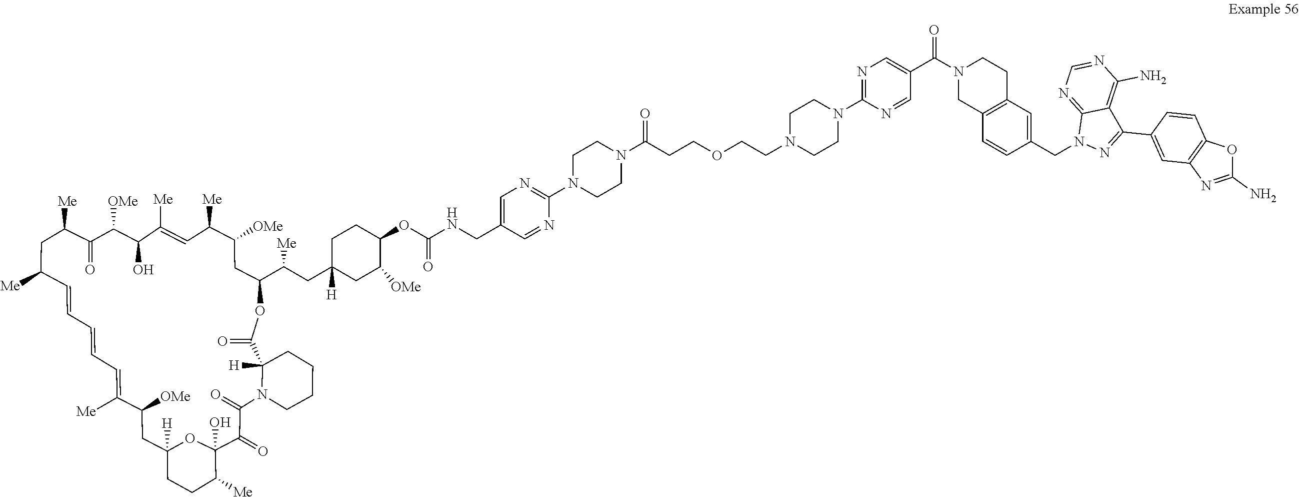 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

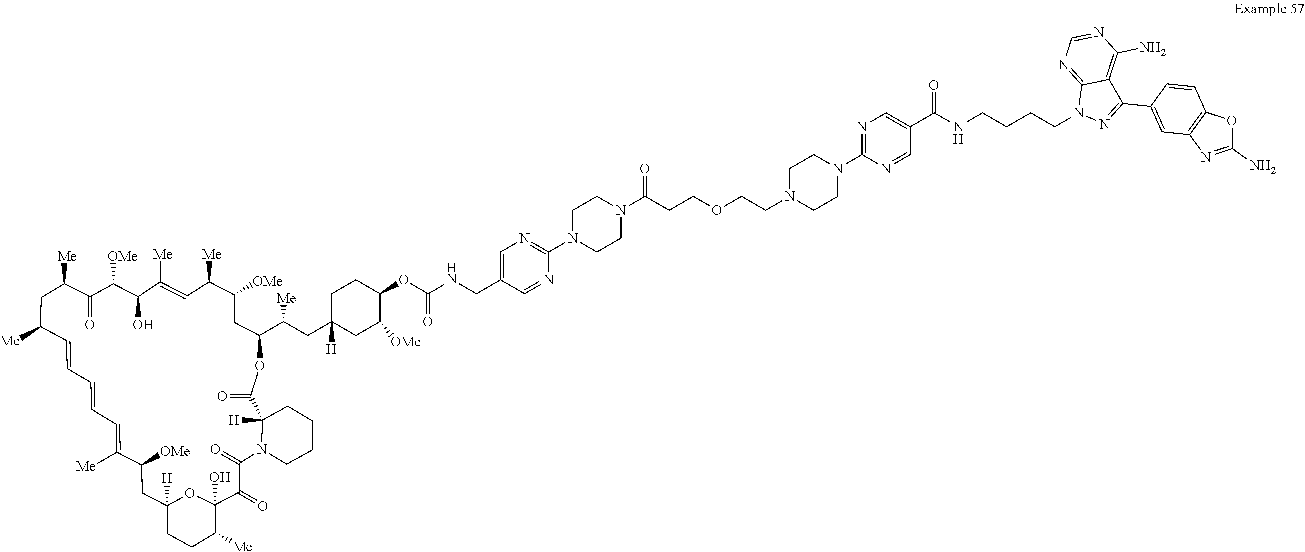 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

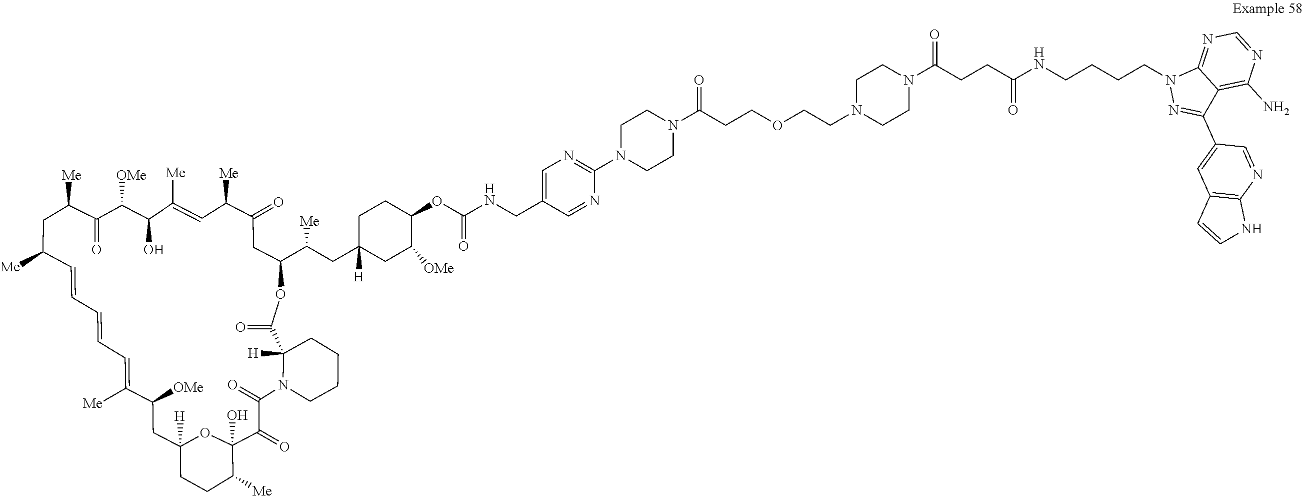 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)NCCCCn6nc(-c7cnc8[nH]ccc8c7)c7c(N)ncnc76)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

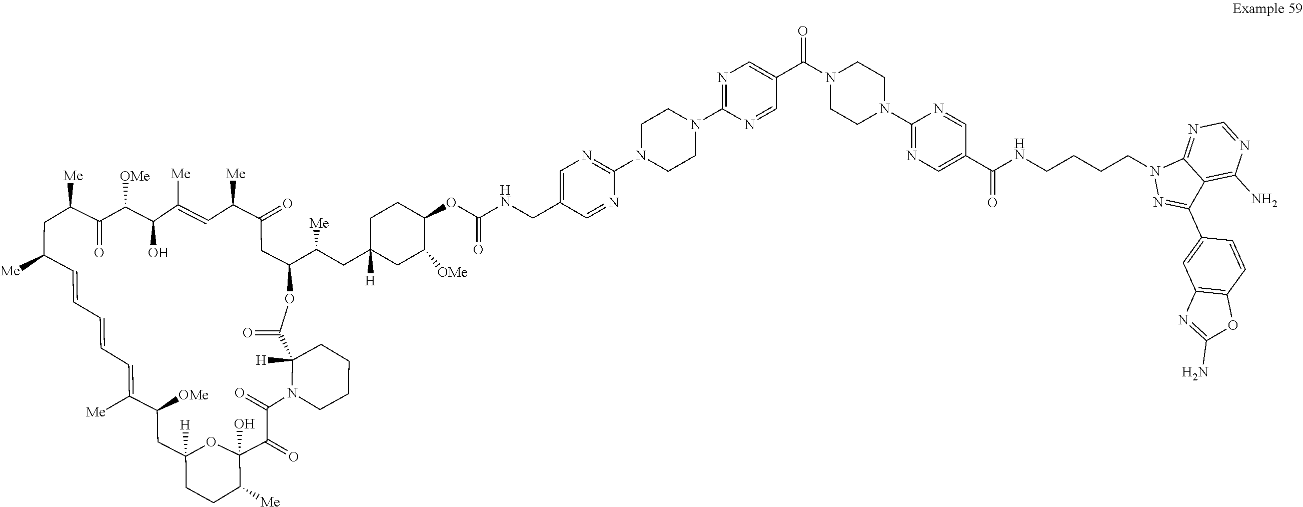 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

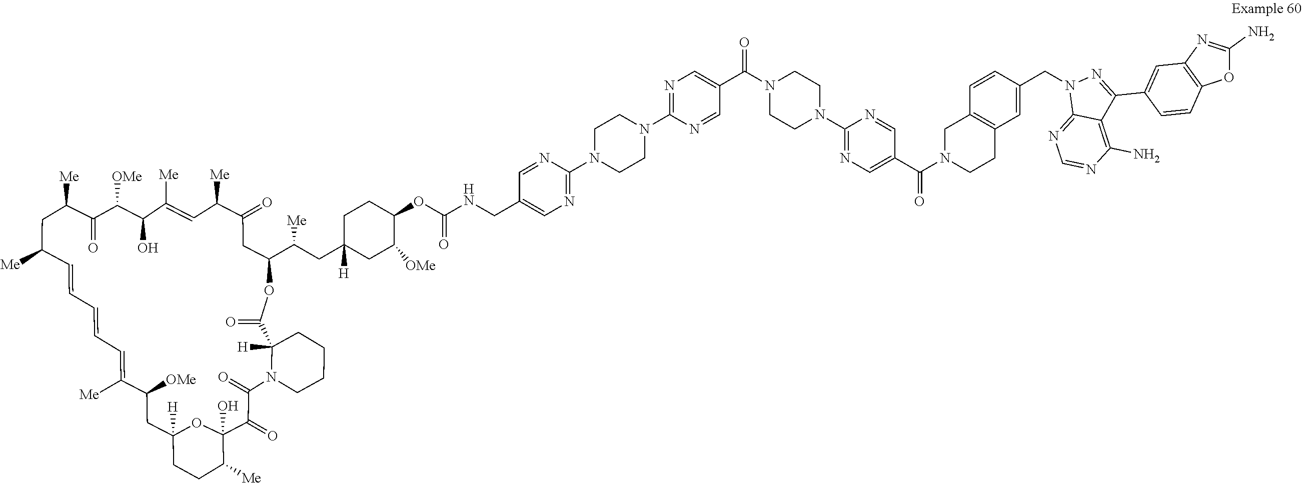 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11ccc%12oc(N)nc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

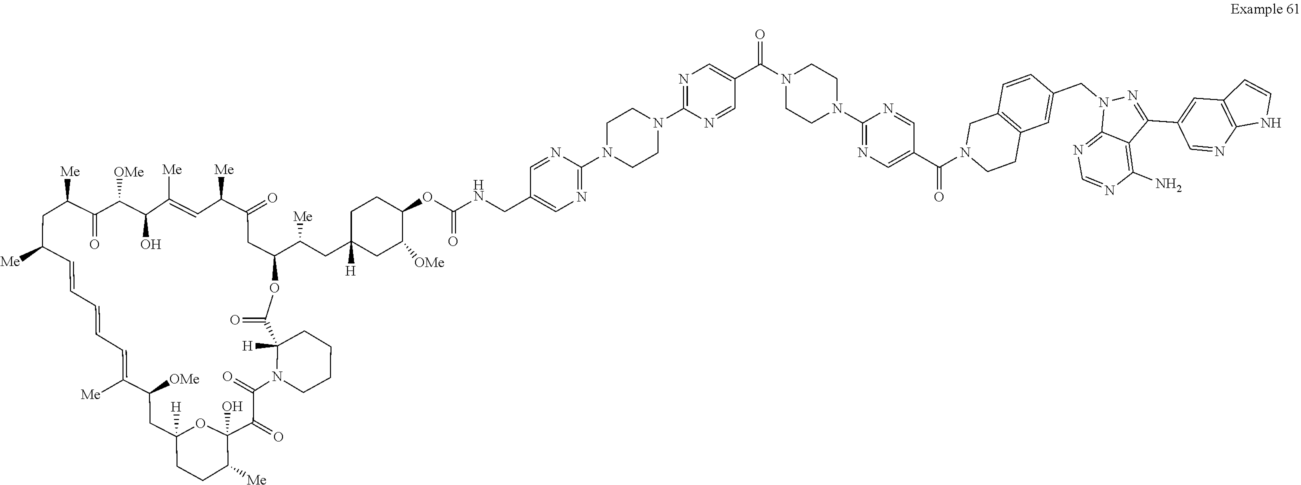 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11cnc%12[nH]ccc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

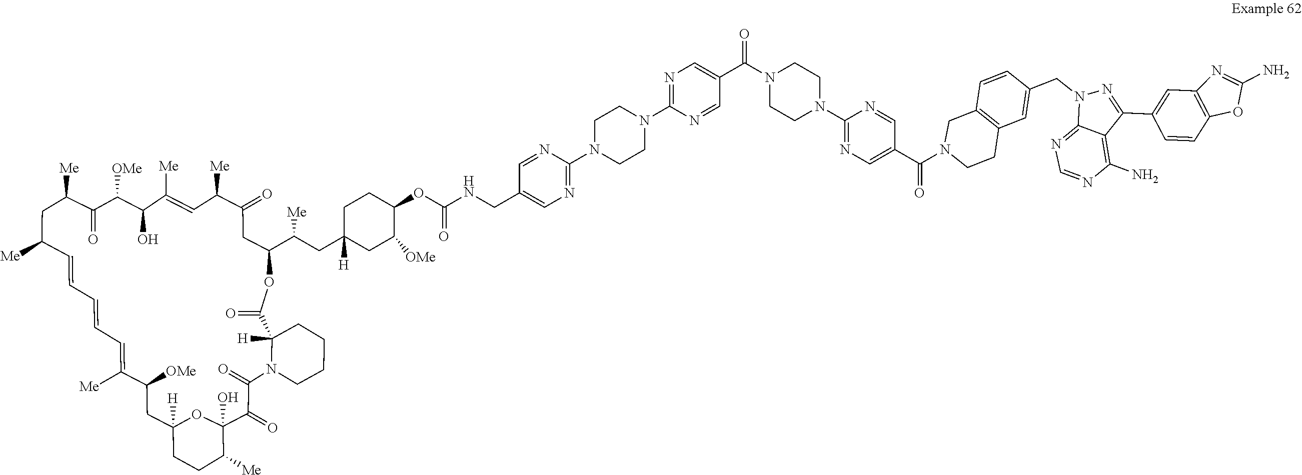 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11ccc%12oc(N)nc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

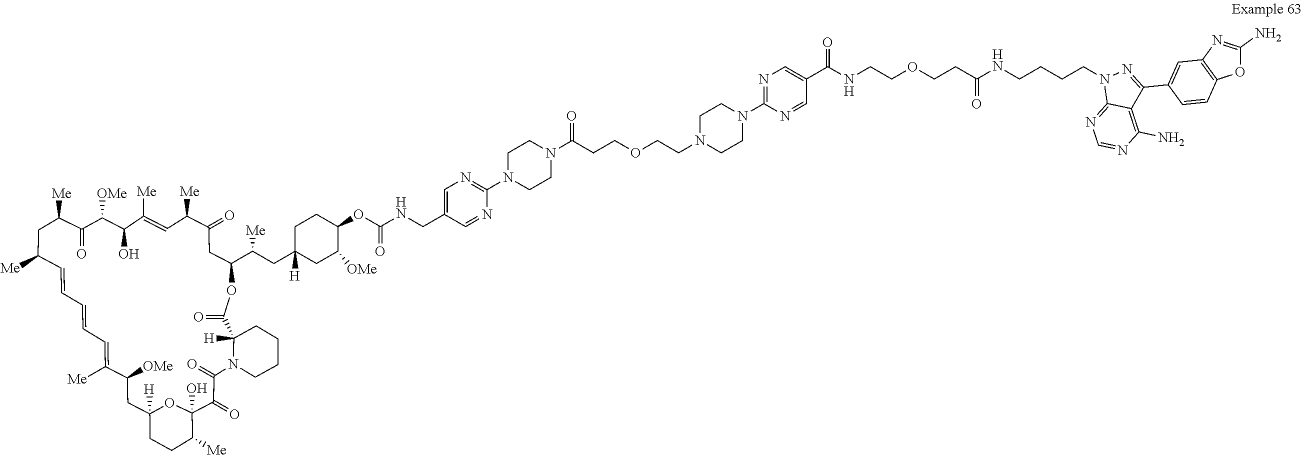 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)NCCCCn7nc(-c8ccc9oc(N)nc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

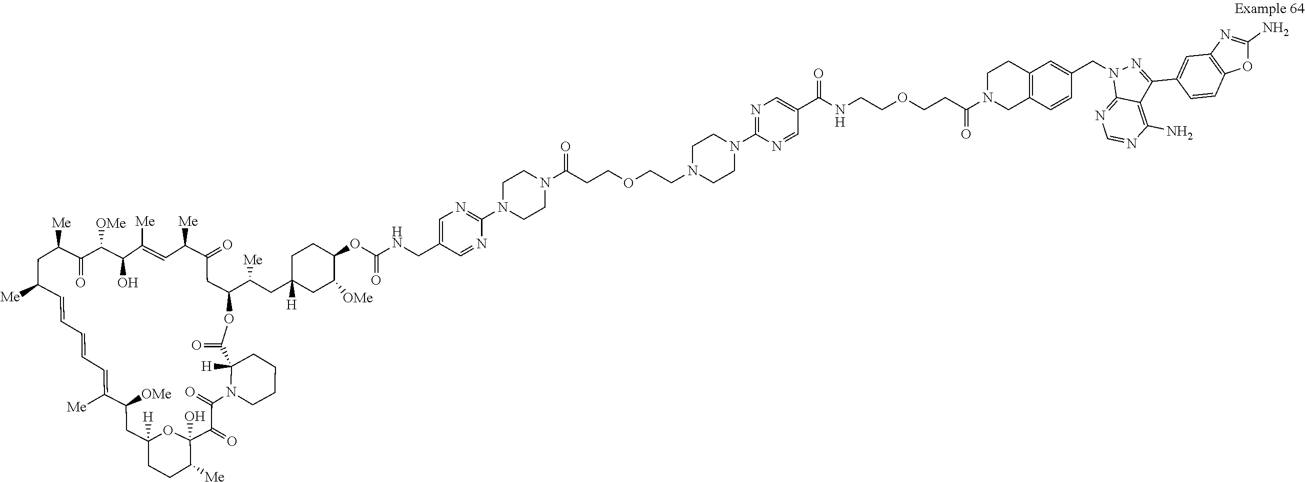 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

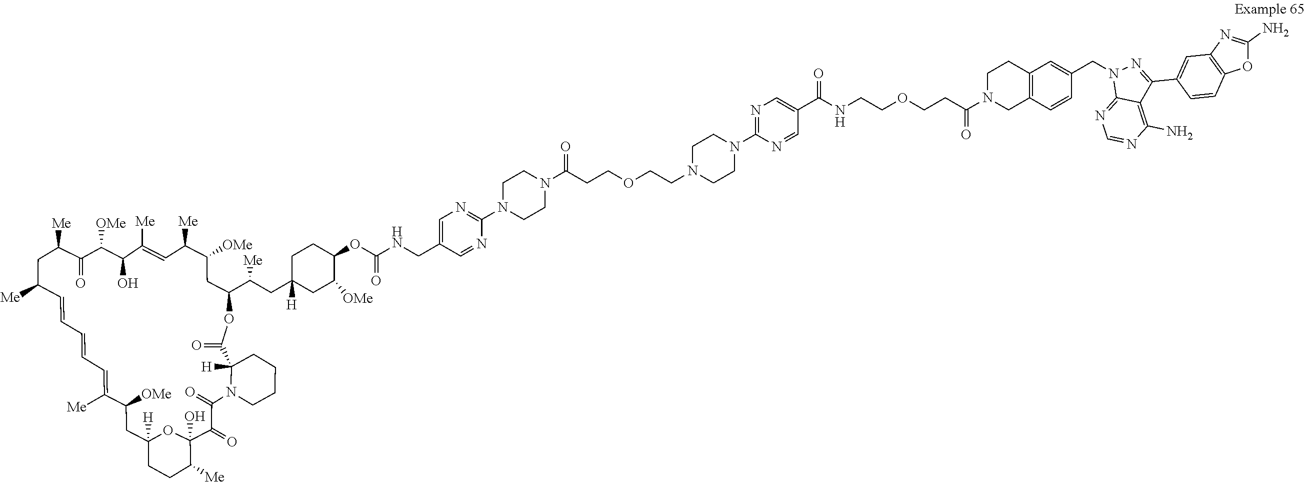 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

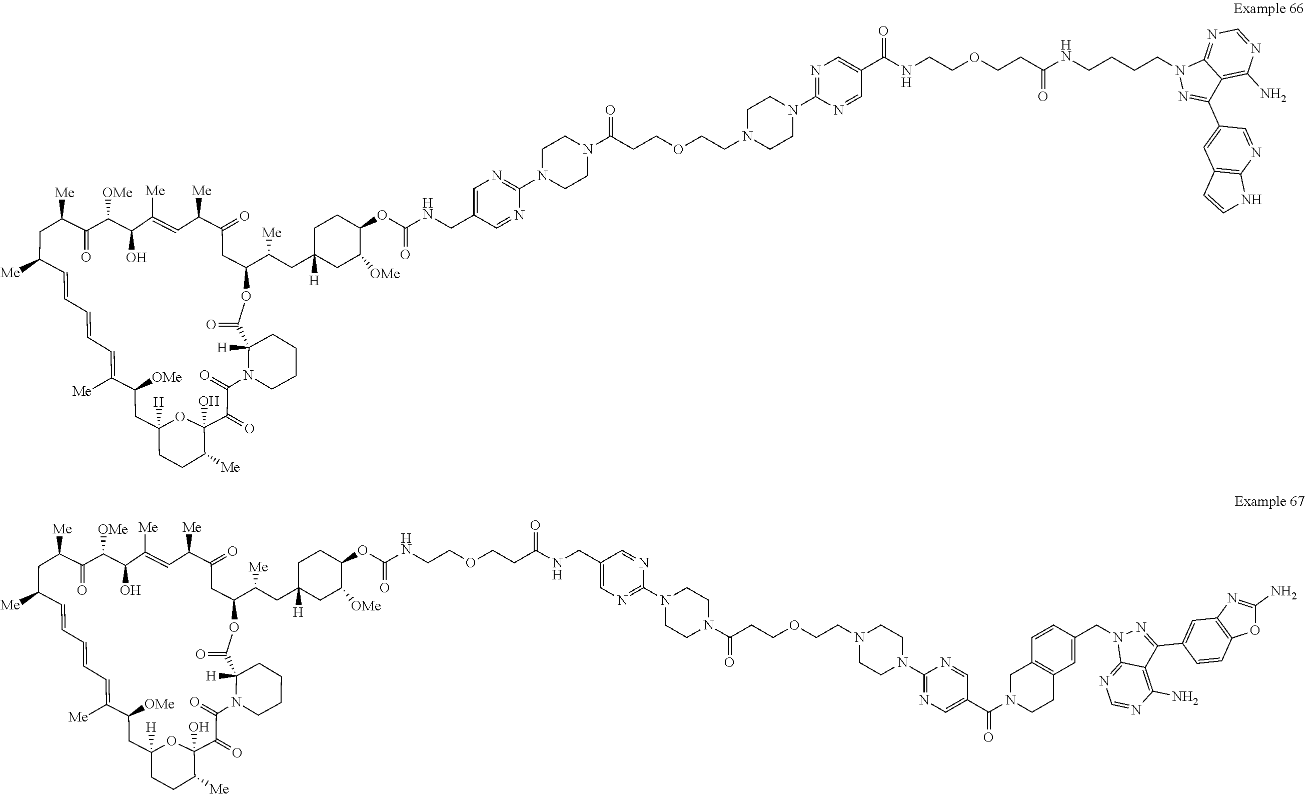

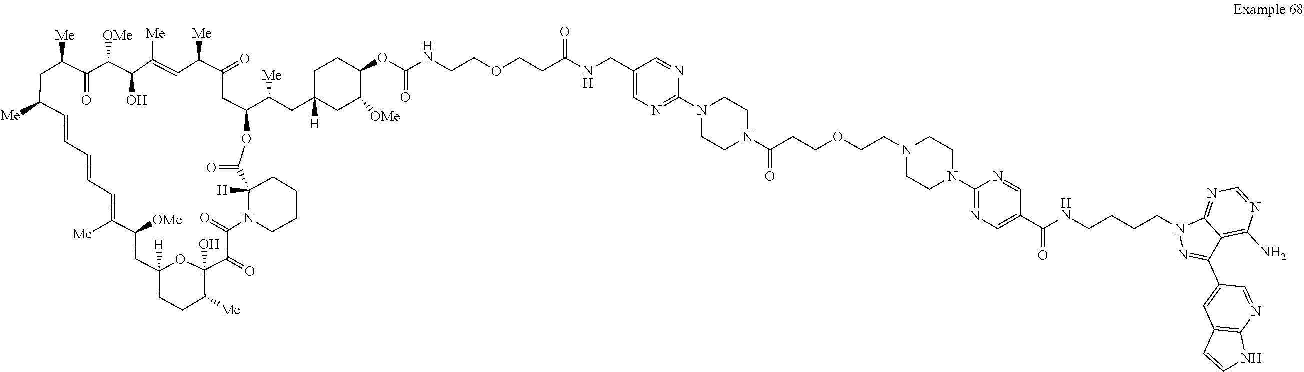 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)NCCCCn7nc(-c8cnc9[nH]ccc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

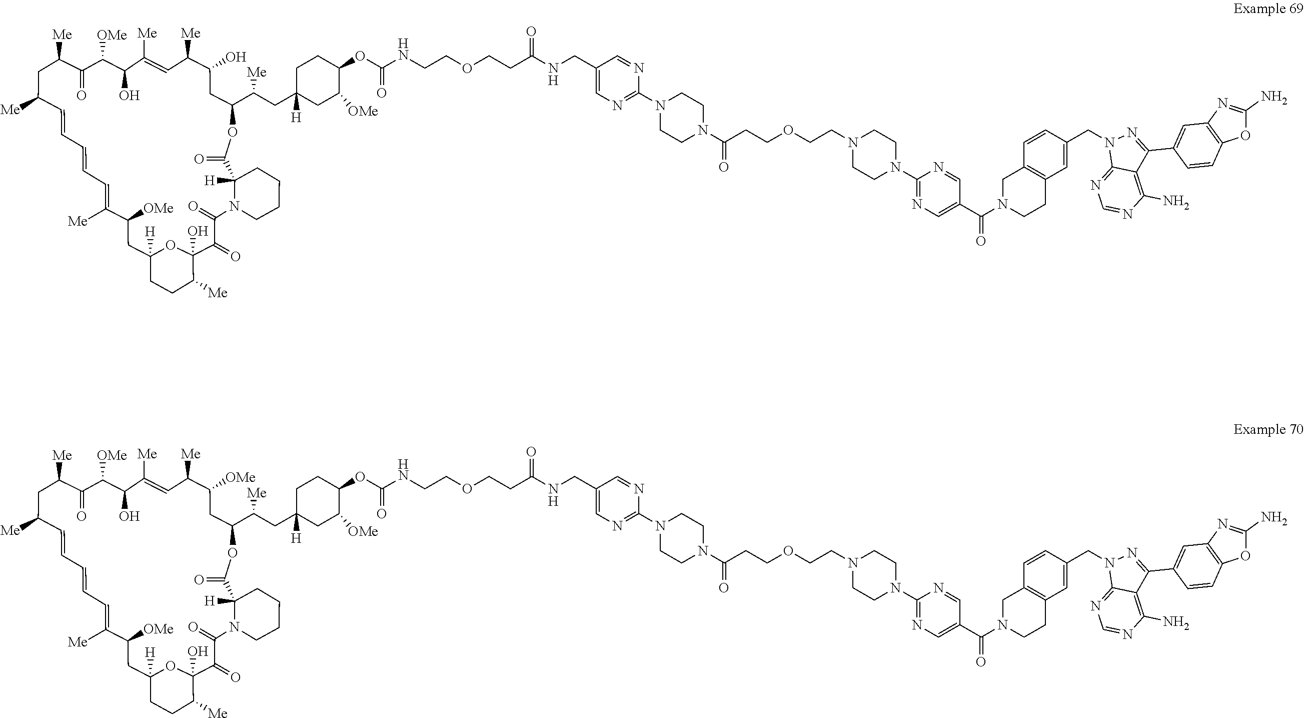

or a pharmaceutically acceptable salt or tautomer thereof.

Embodiment II-73. A compound selected from the group consisting of:

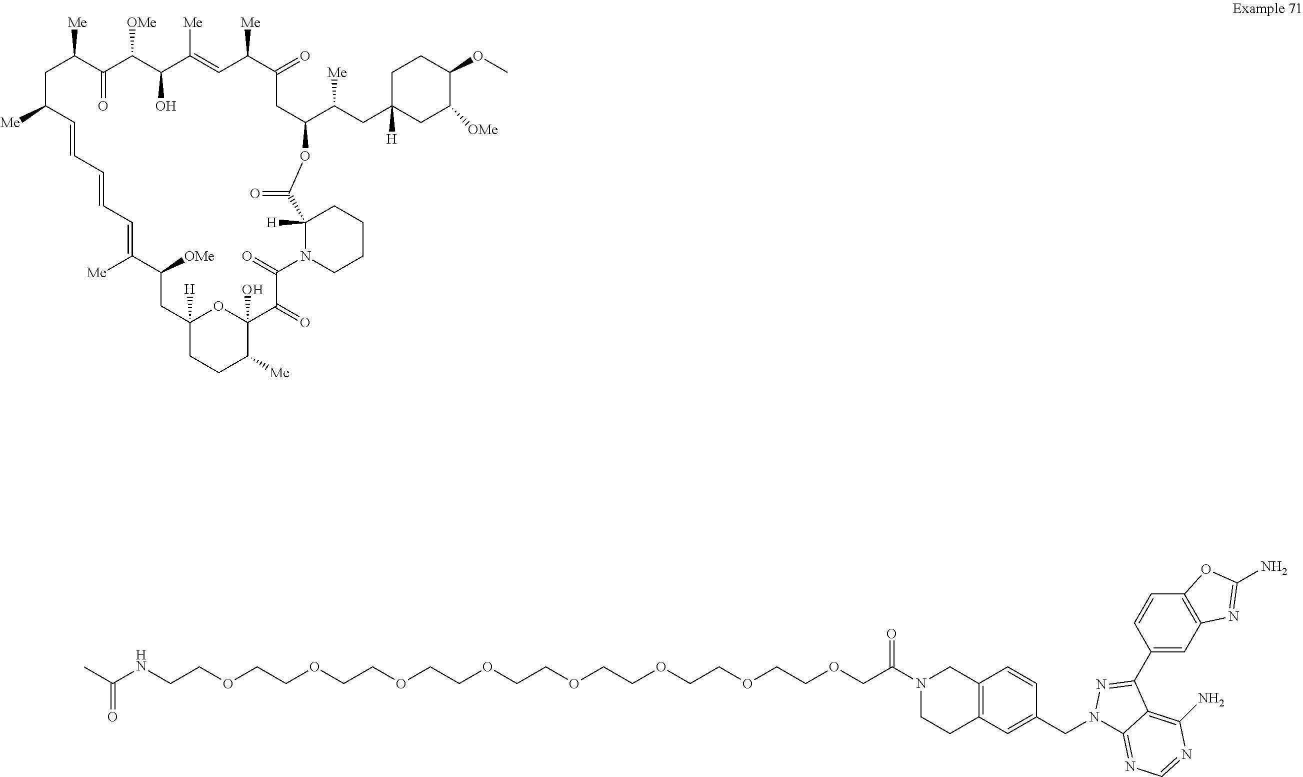 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC)[C@H](OC)C2)CC(=O)[C@H](C)/C=C(\C)[C@@H](O)[C@@H](OC)C(=O)[C@H](C)C[C@H](C)/C=C/C=C/C=C/1C ; CC(=O)NCCOCCOCCOCCOCCOCCOCCOCCOCC(=O)N1CCc2cc(Cn3nc(-c4ccc5oc(N)nc5c4)c4c(N)ncnc43)ccc2C1]

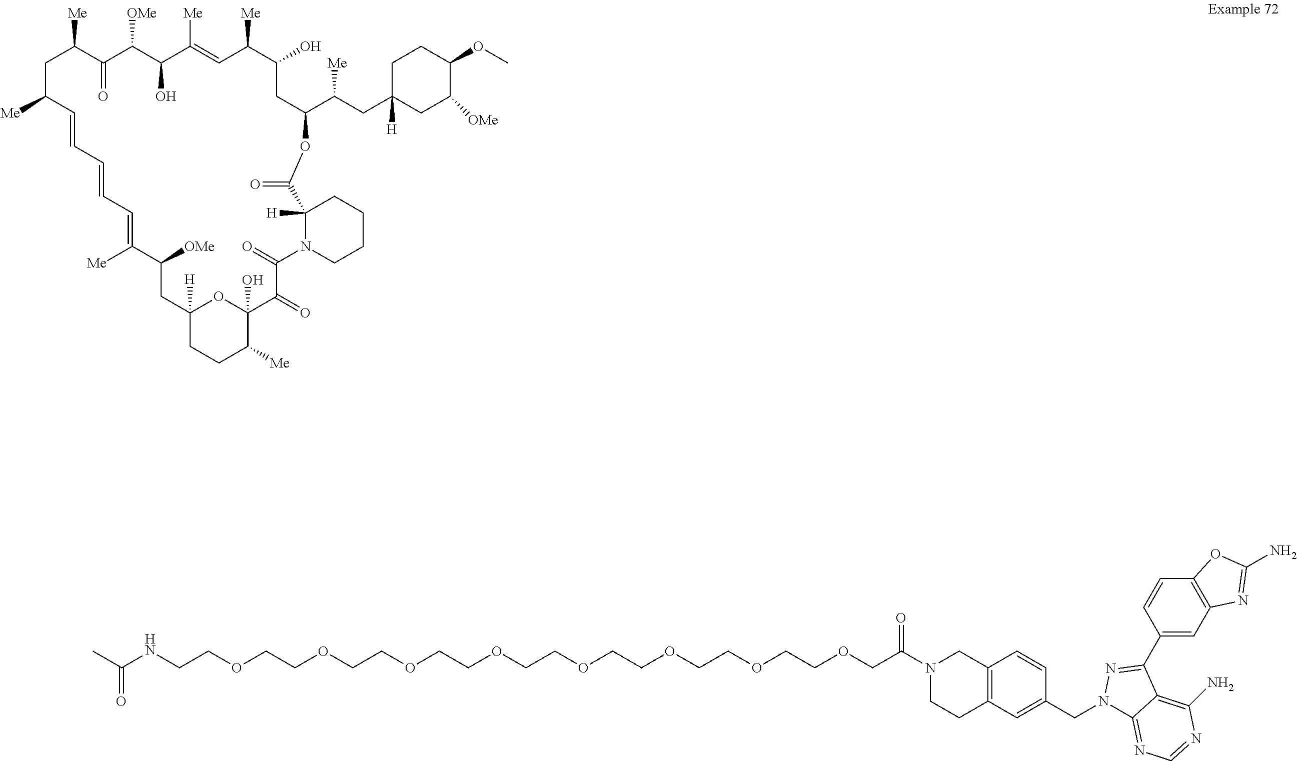 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC)[C@H](OC)C2)C[C@@H](O)[C@H](C)/C=C(\C)[C@@H](O)[C@@H](OC)C(=O)[C@H](C)C[C@H](C)/C=C/C=C/C=C/1C ; CC(=O)NCCOCCOCCOCCOCCOCCOCCOCCOCC(=O)N1CCc2cc(Cn3nc(-c4ccc5oc(N)nc5c4)c4c(N)ncnc43)ccc2C1]

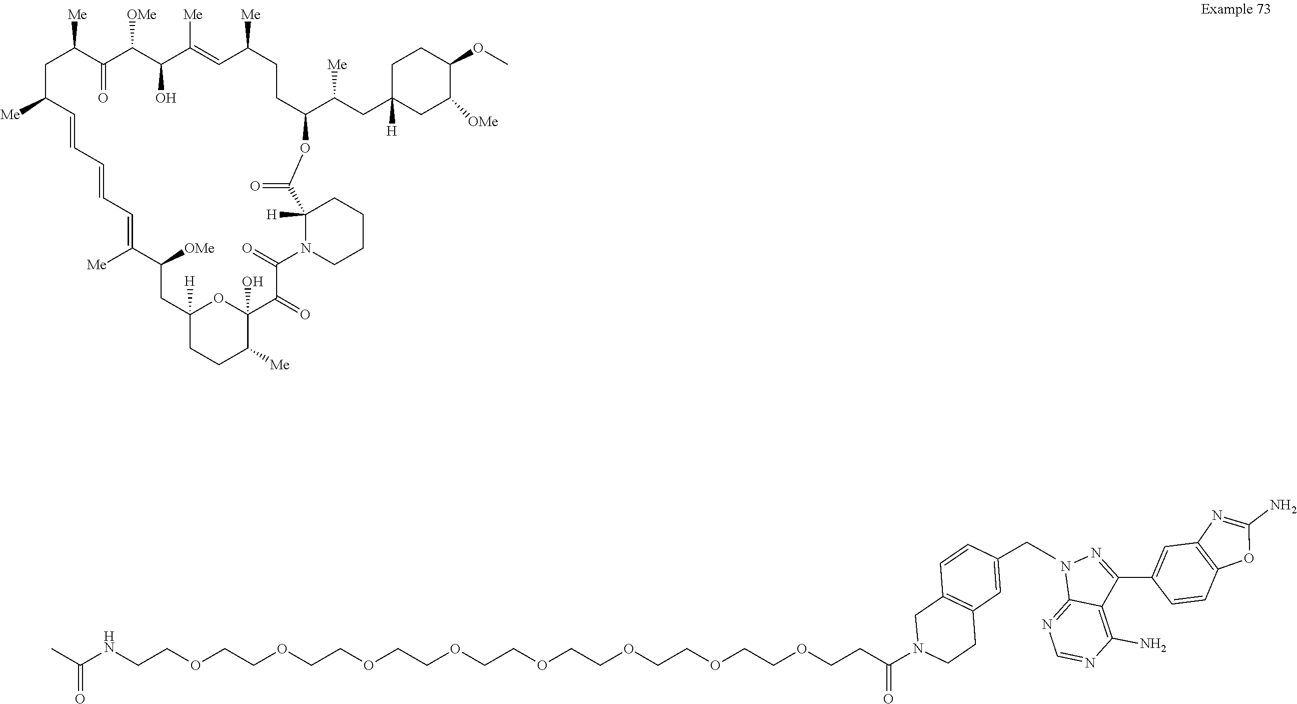

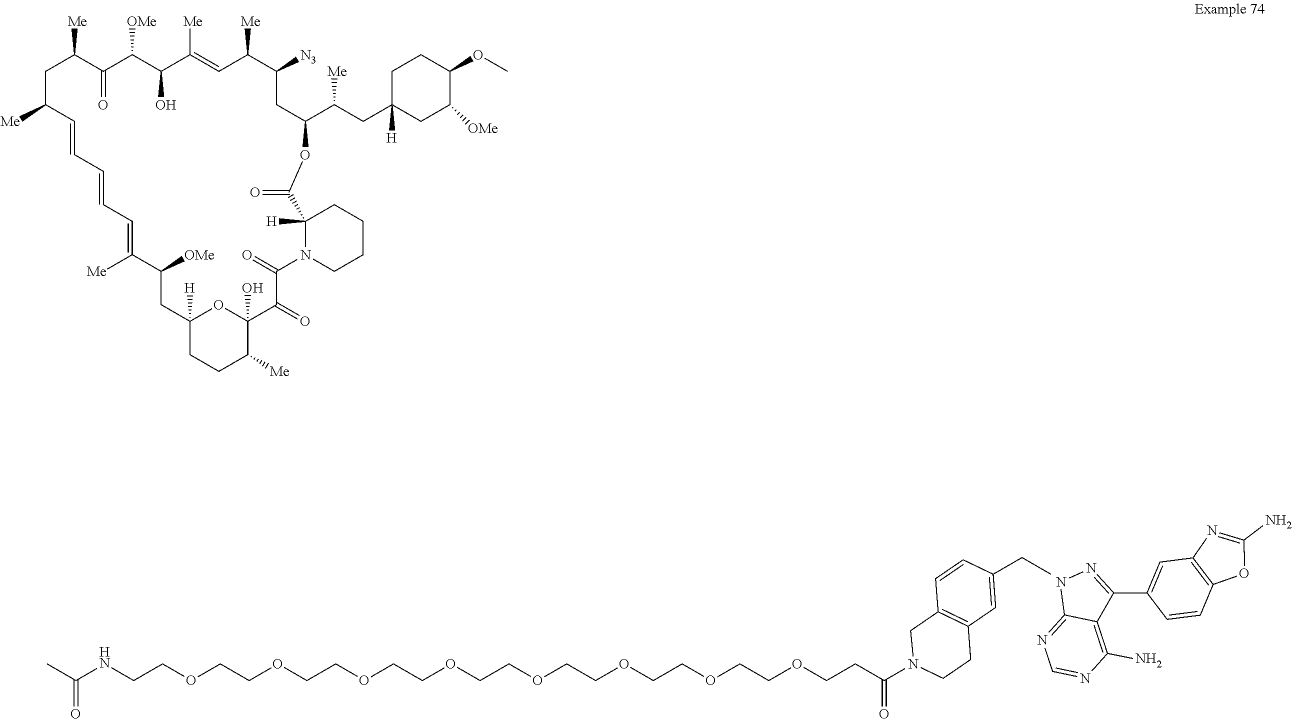

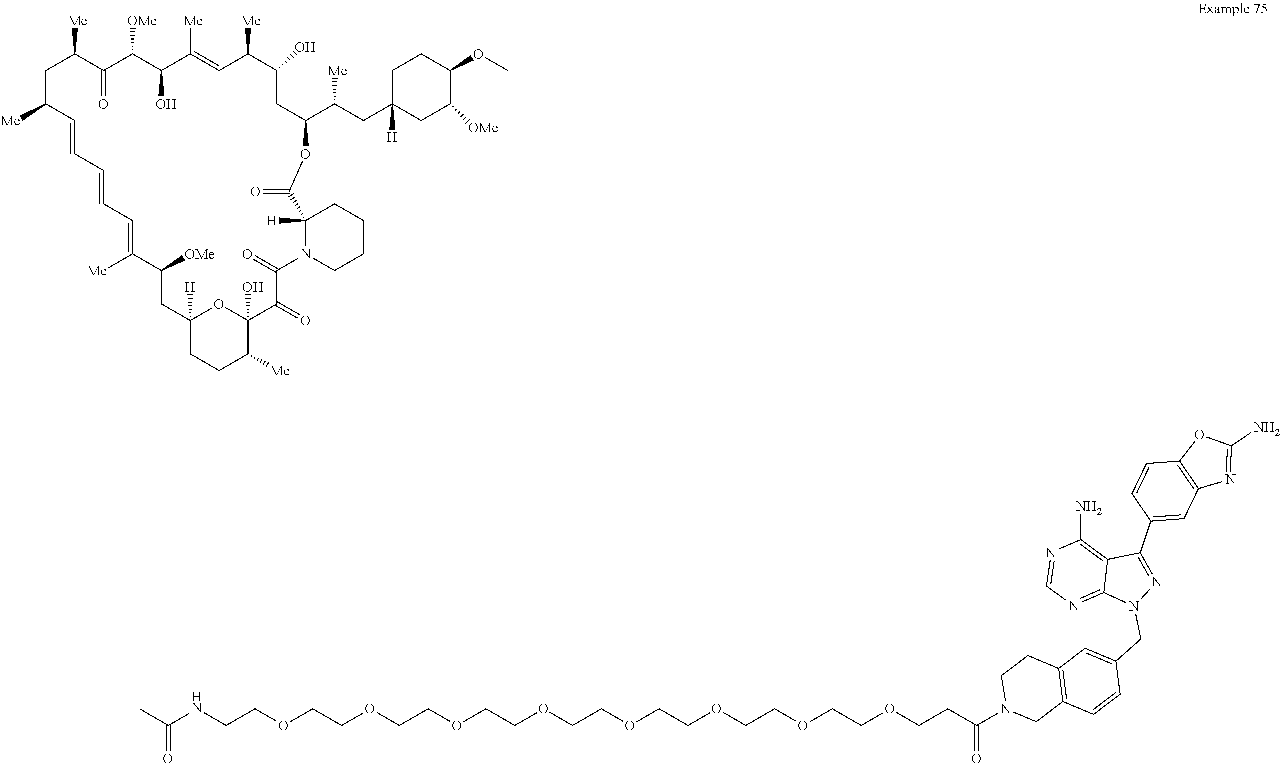 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC)[C@H](OC)C2)C[C@@H](O)[C@H](C)/C=C(\C)[C@@H](O)[C@@H](OC)C(=O)[C@H](C)C[C@H](C)/C=C/C=C/C=C/1C ; CC(=O)NCCOCCOCCOCCOCCOCCOCCOCCOCCC(=O)N1CCc2cc(Cn3nc(-c4ccc5oc(N)nc5c4)c4c(N)ncnc43)ccc2C1]

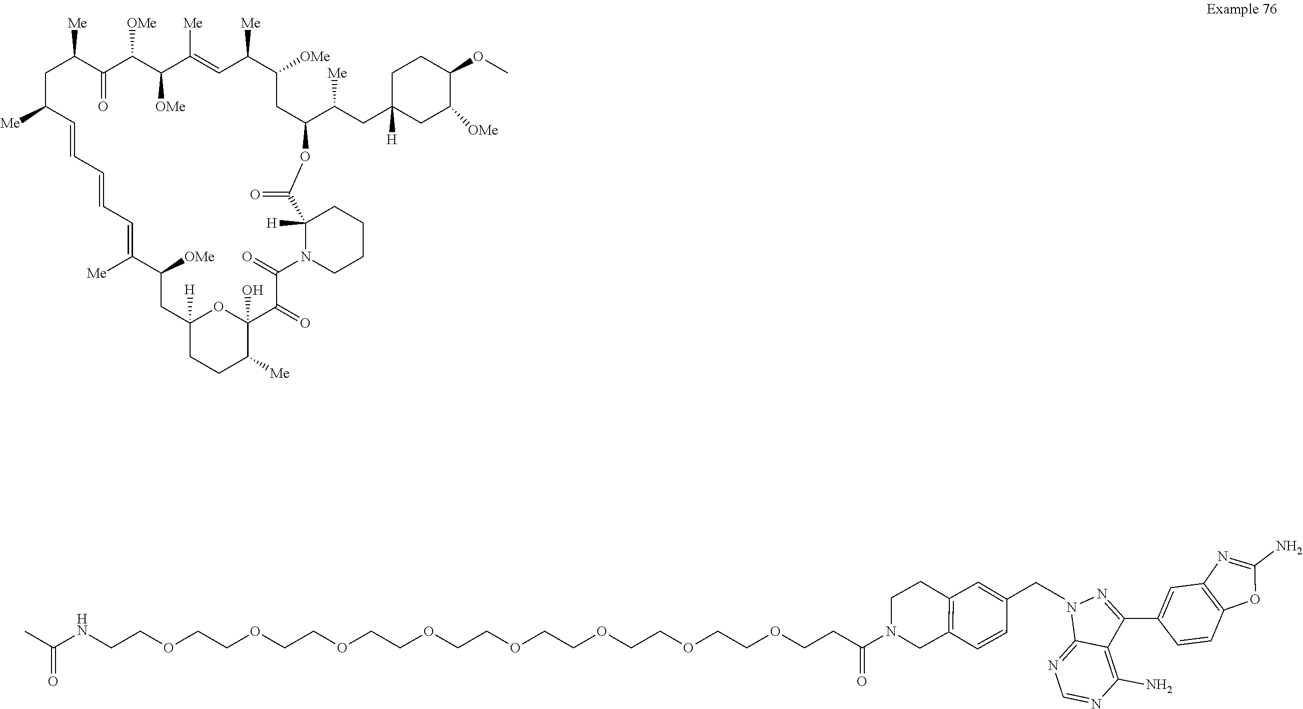

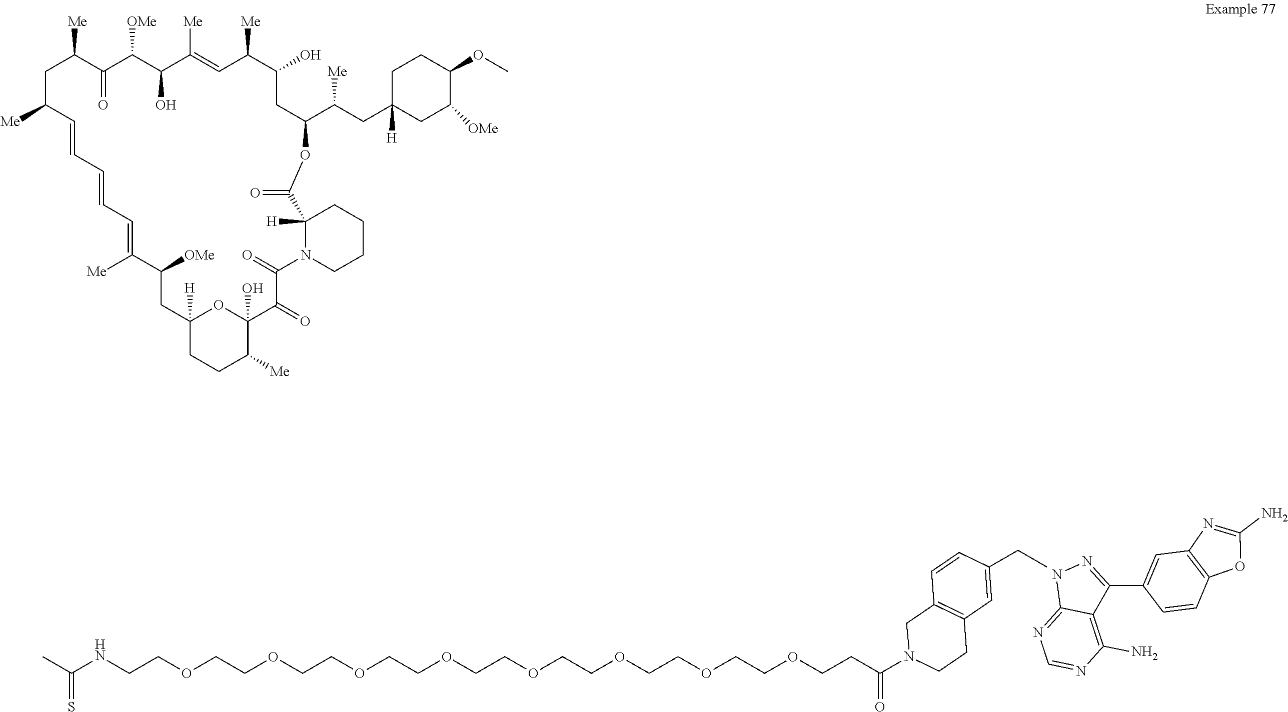

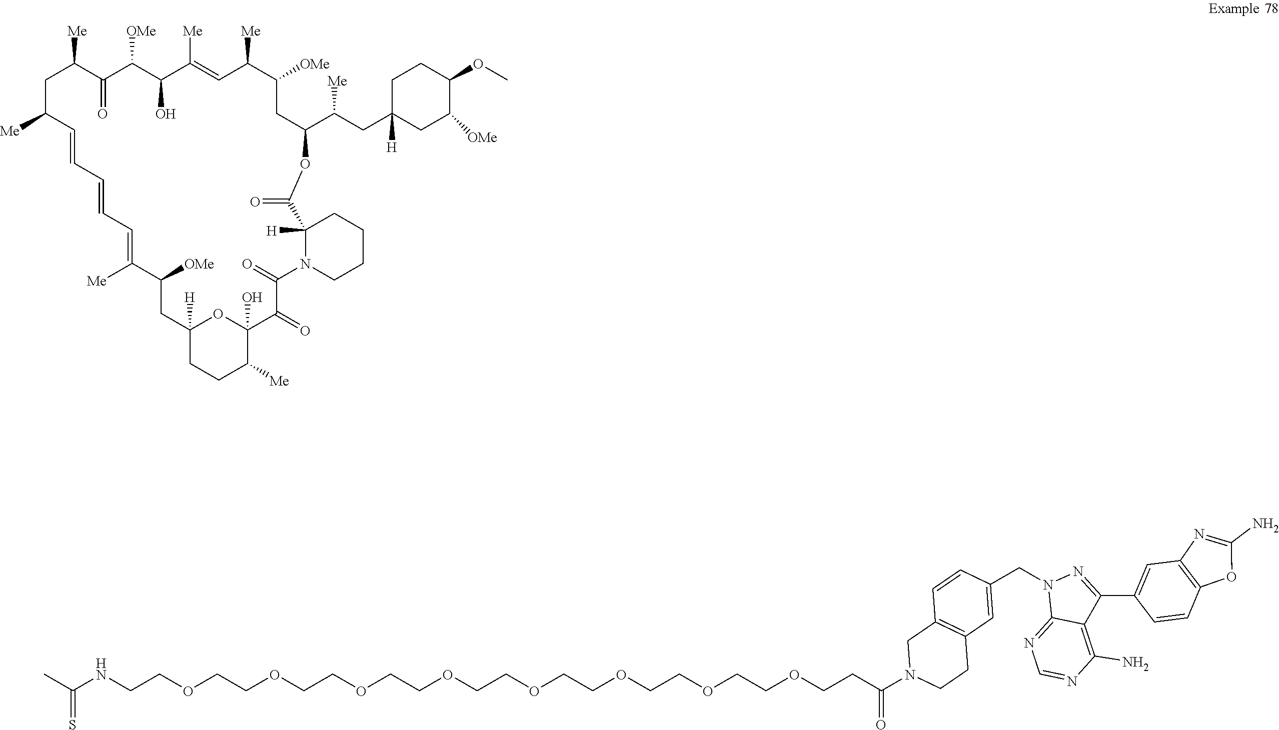

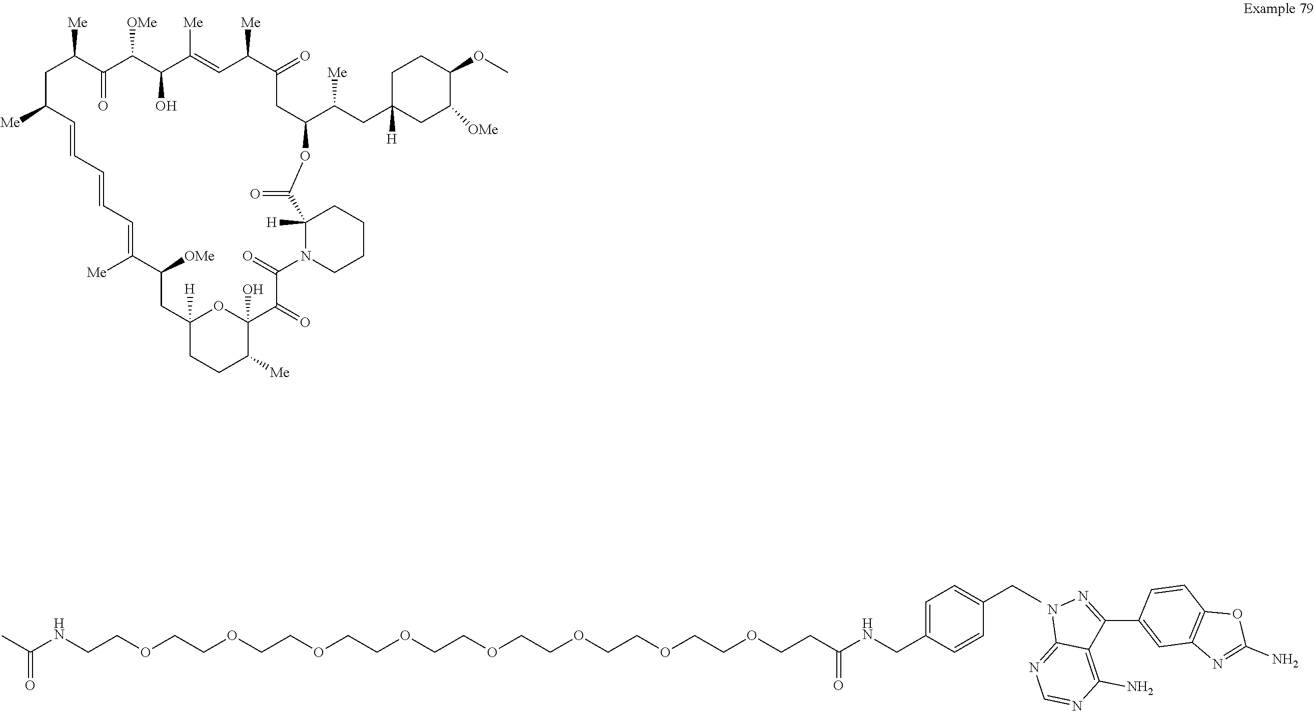

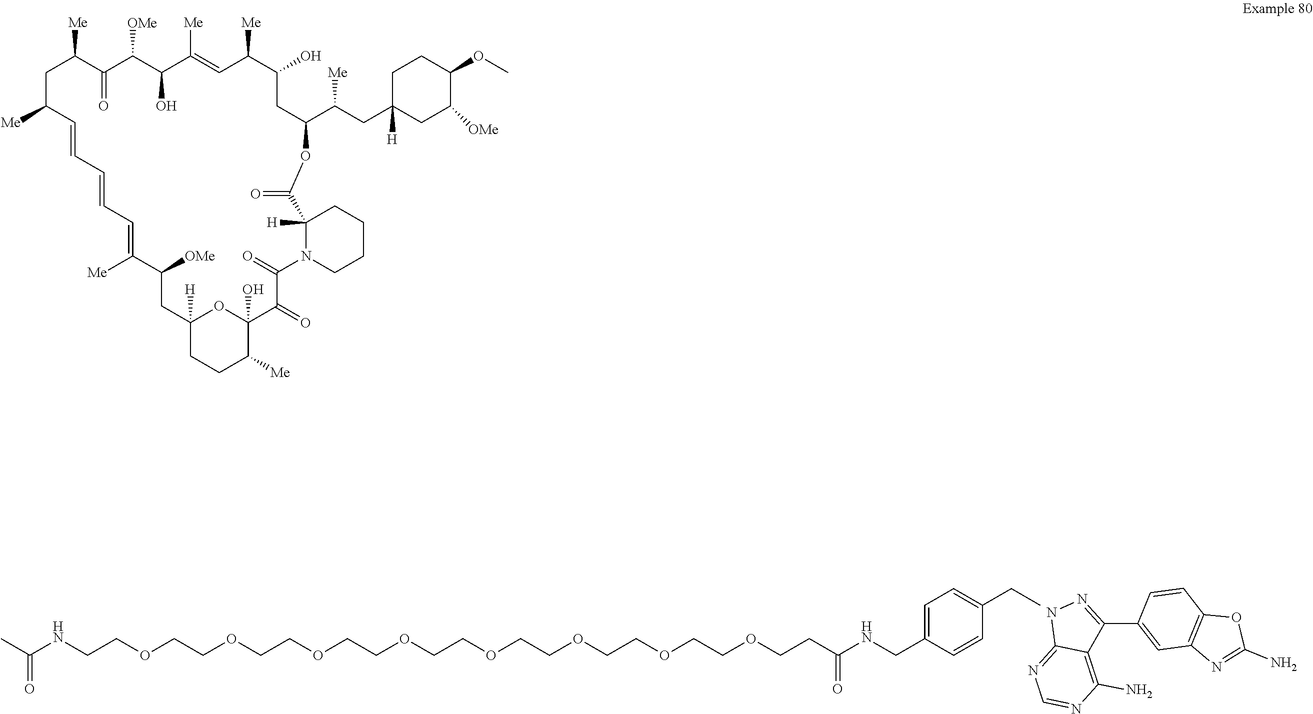

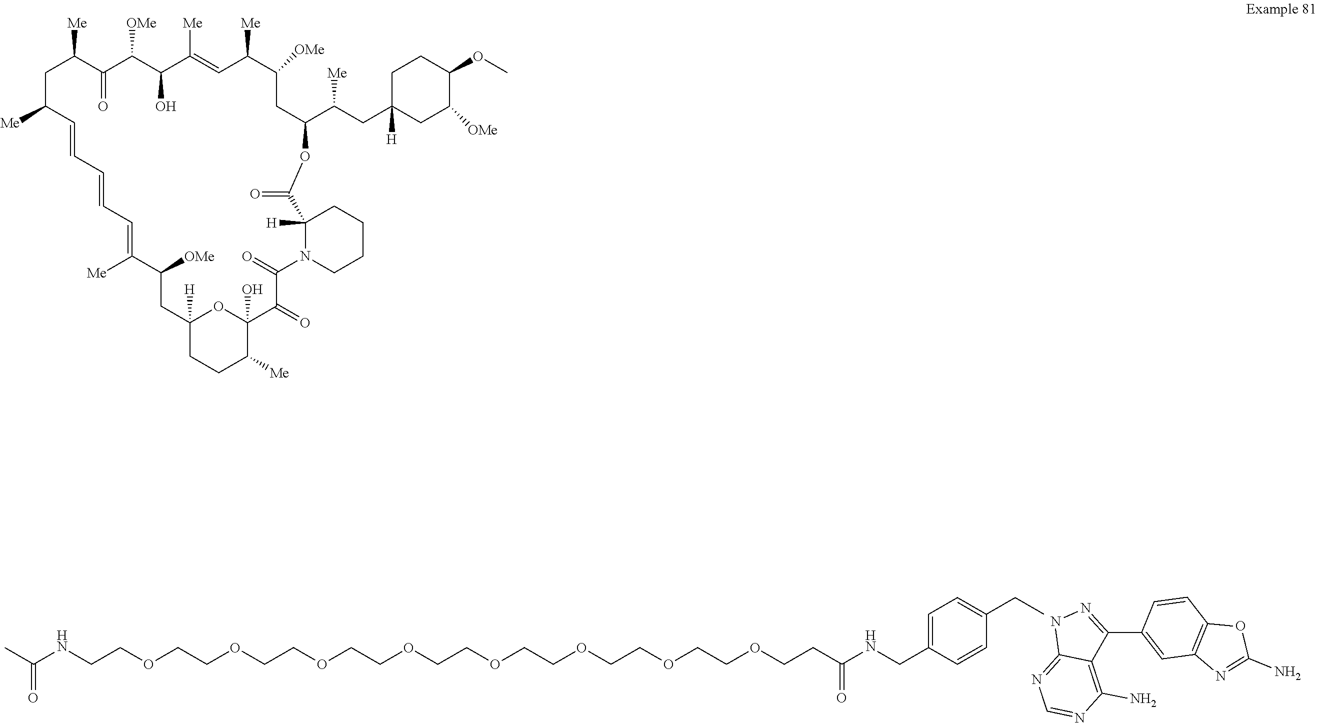

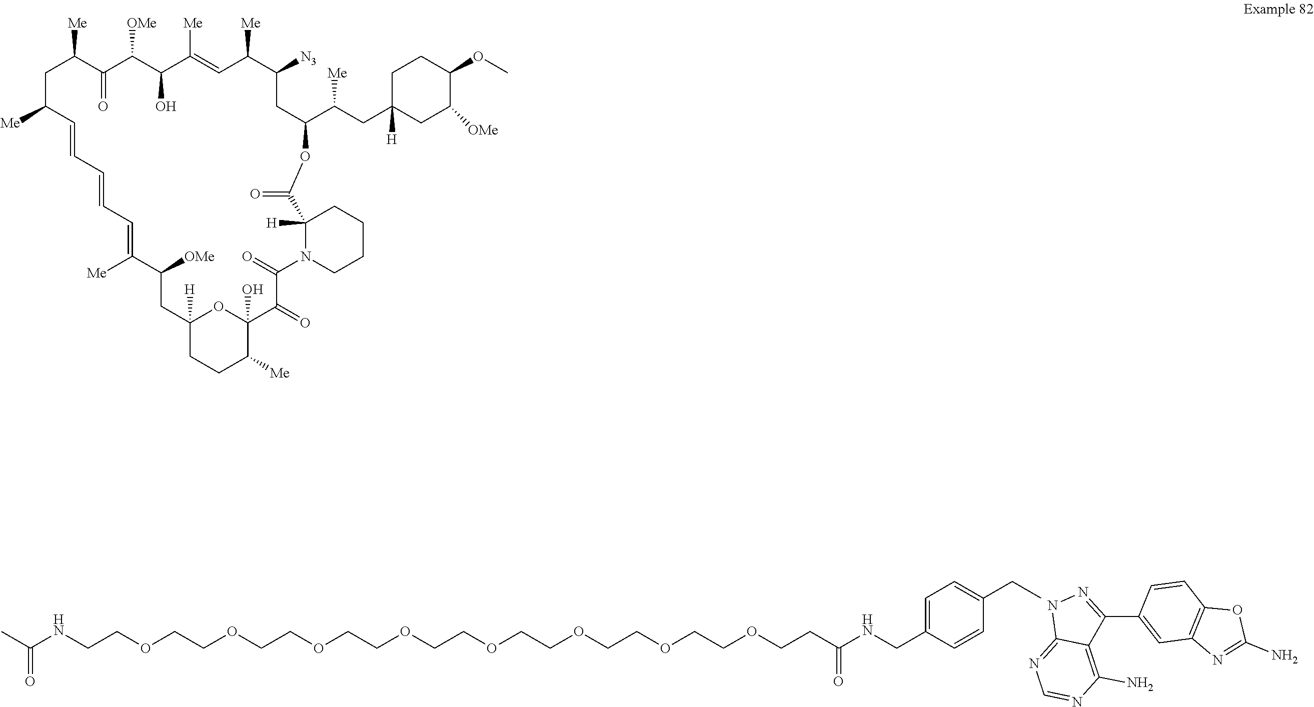

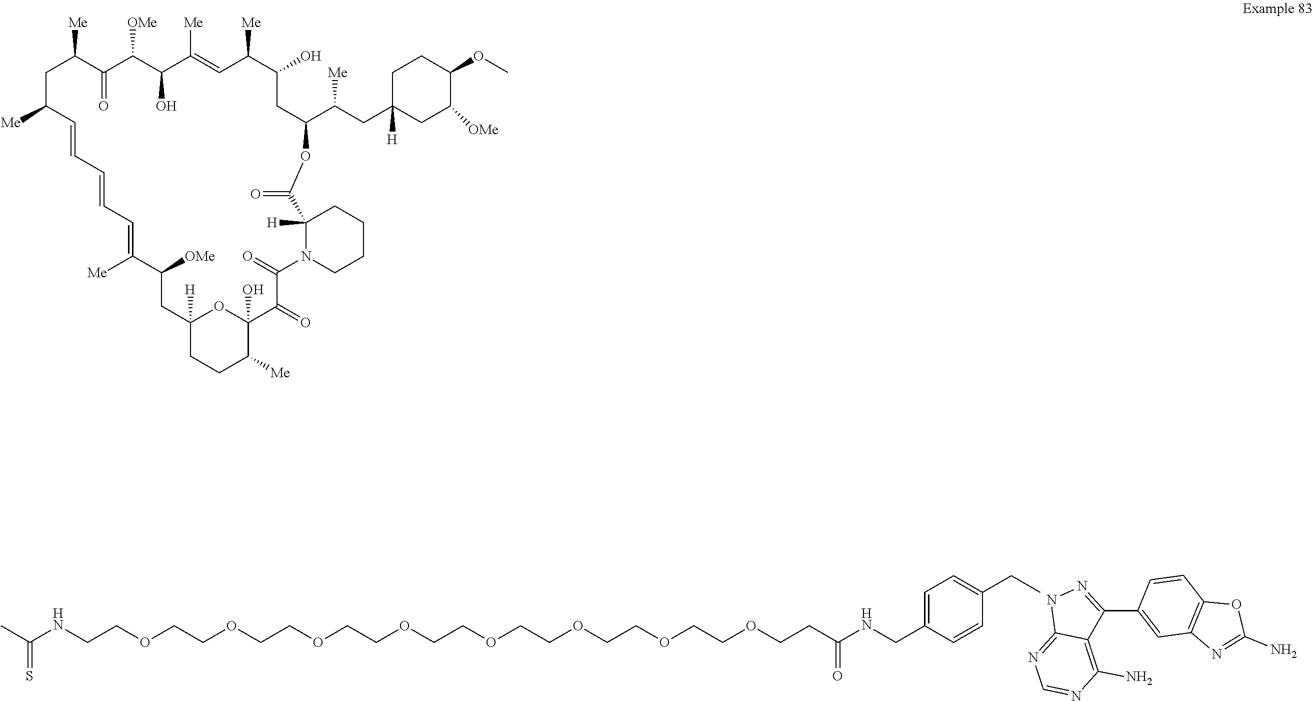

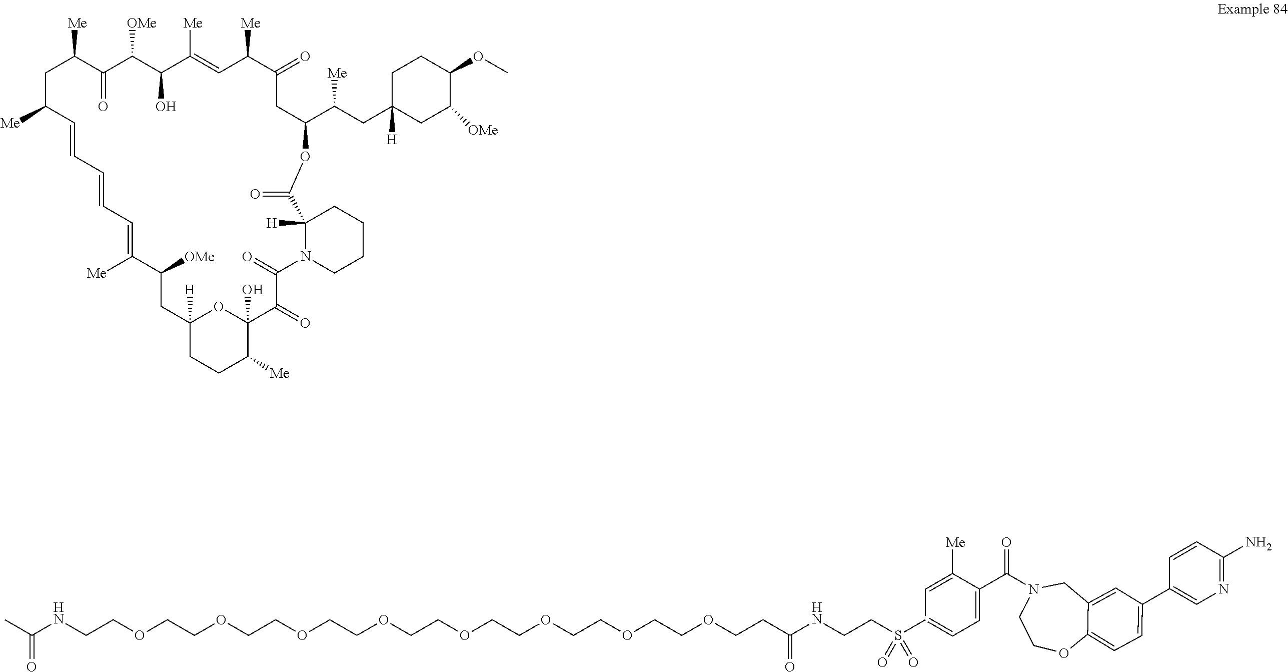

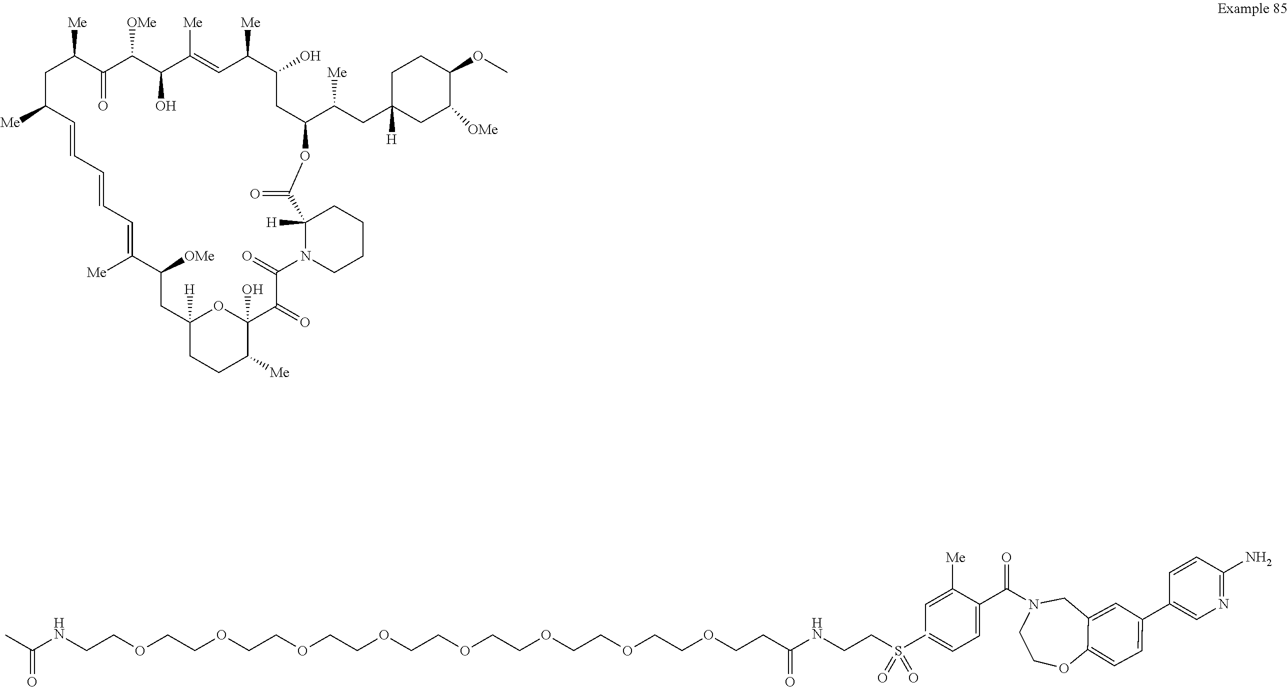

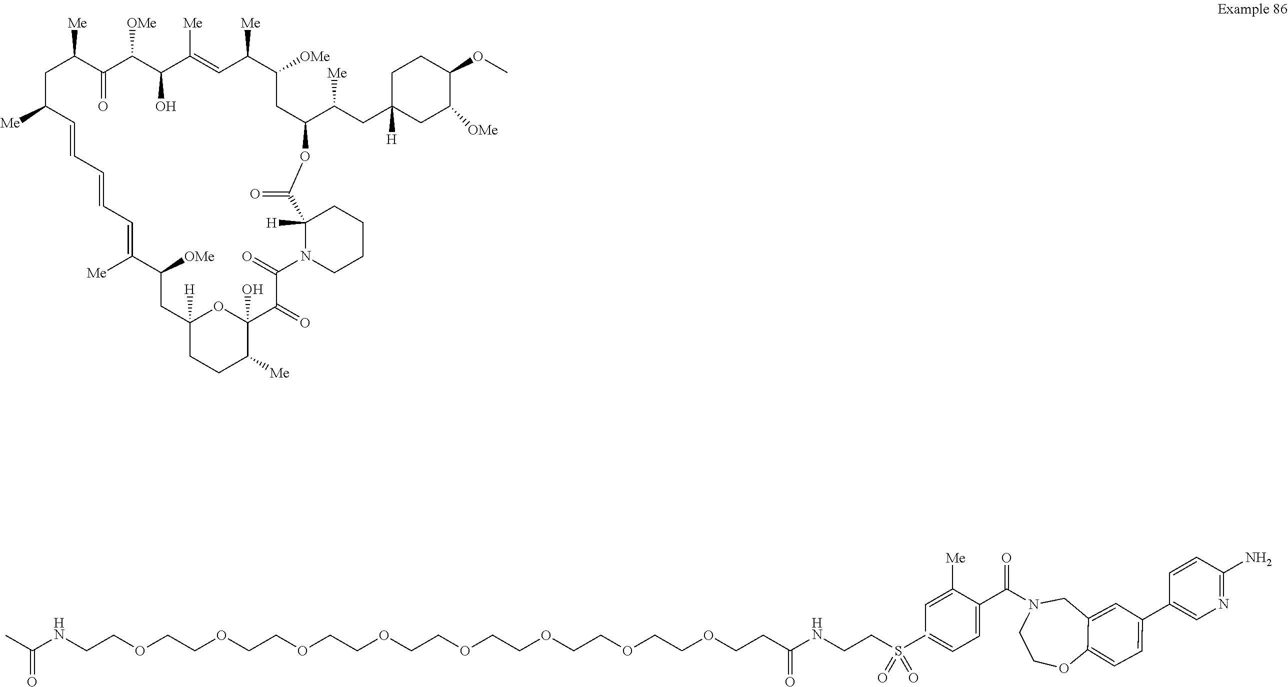

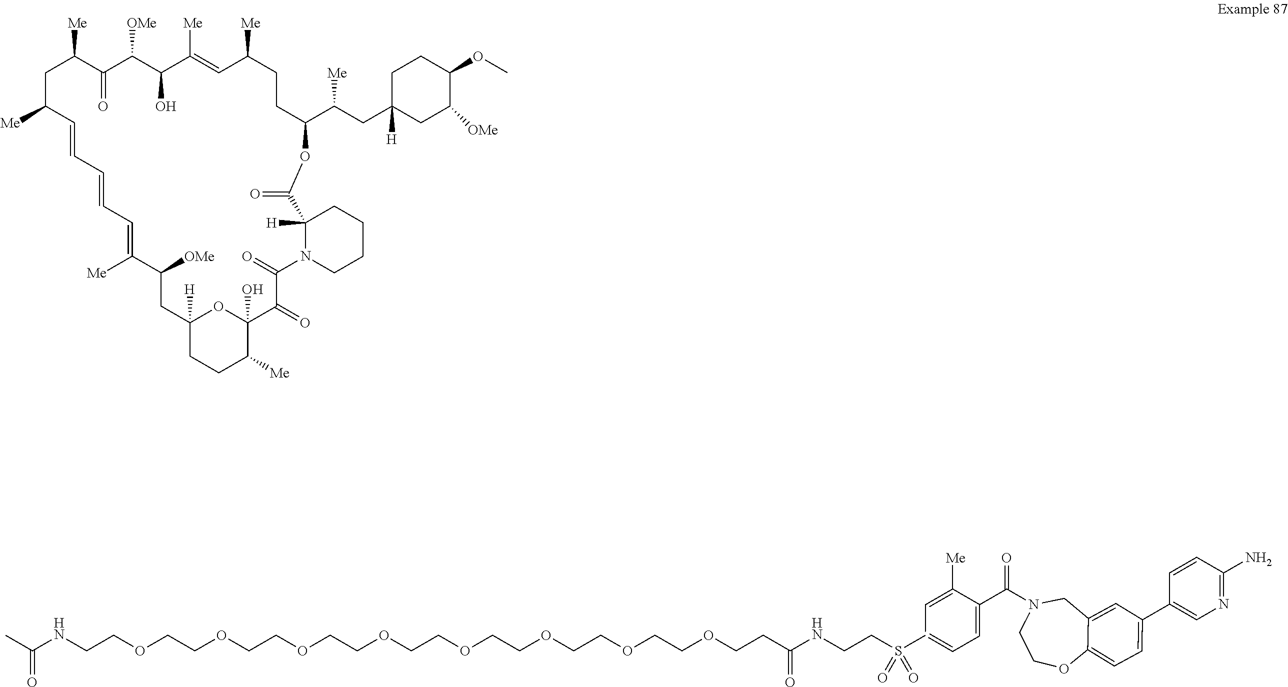

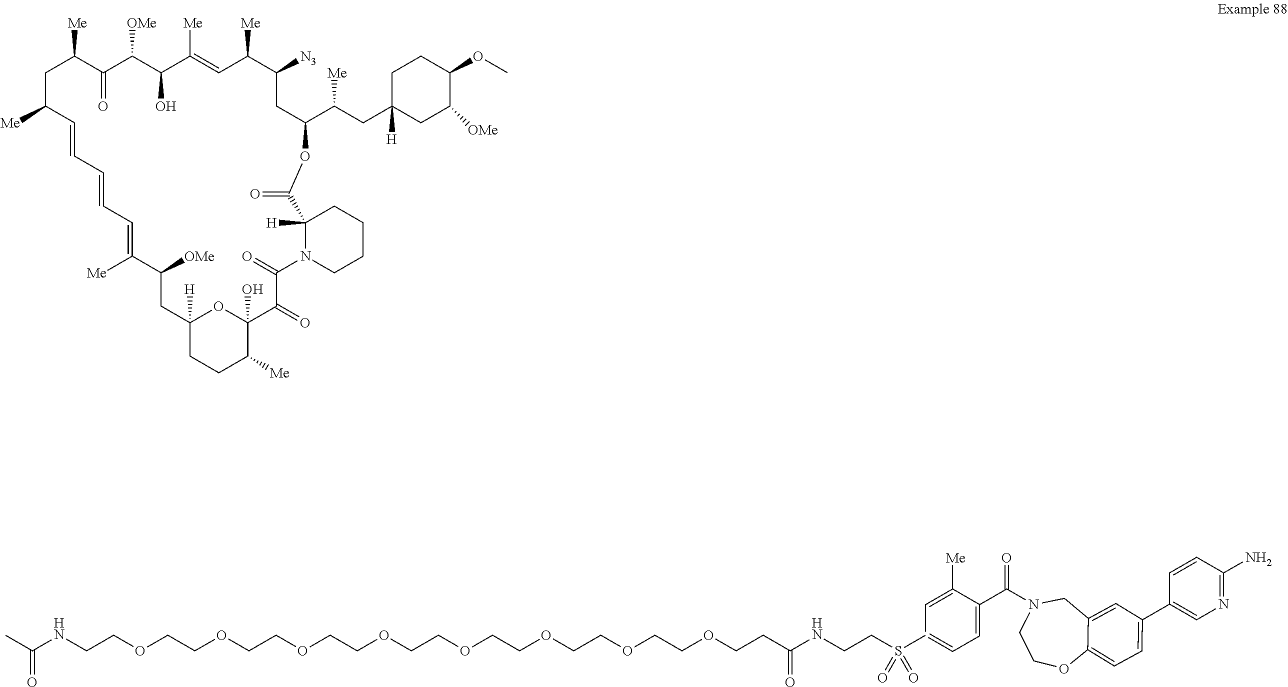

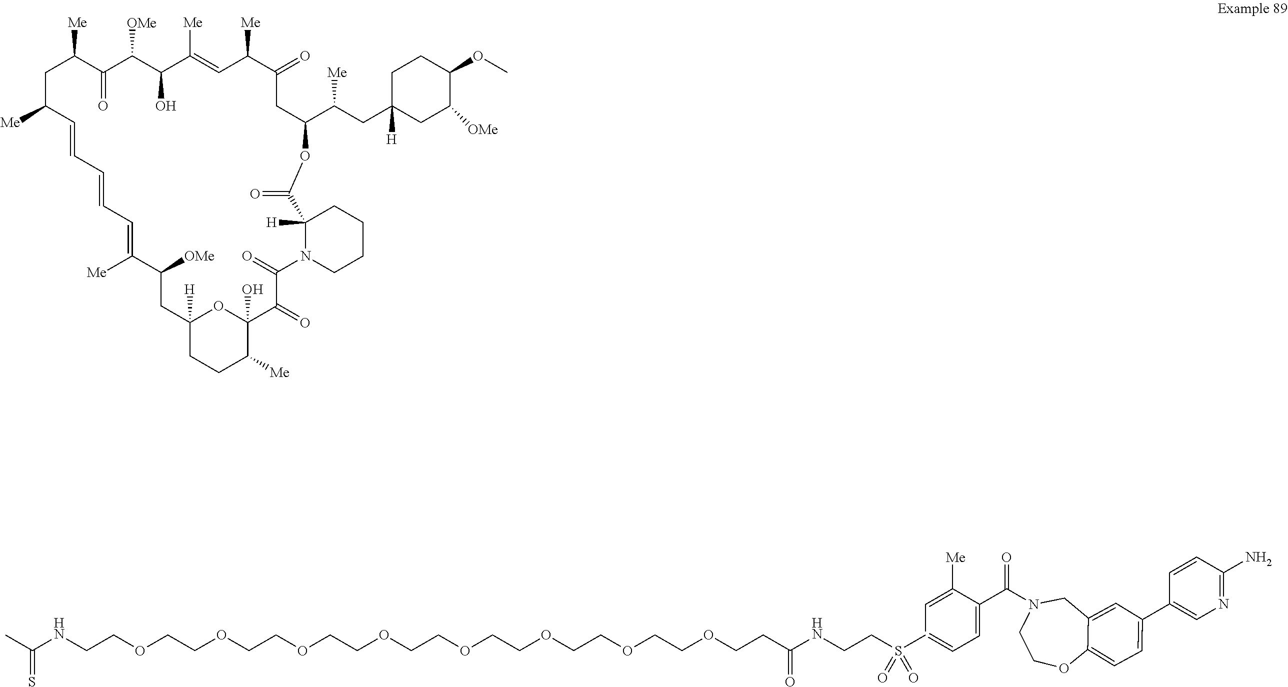

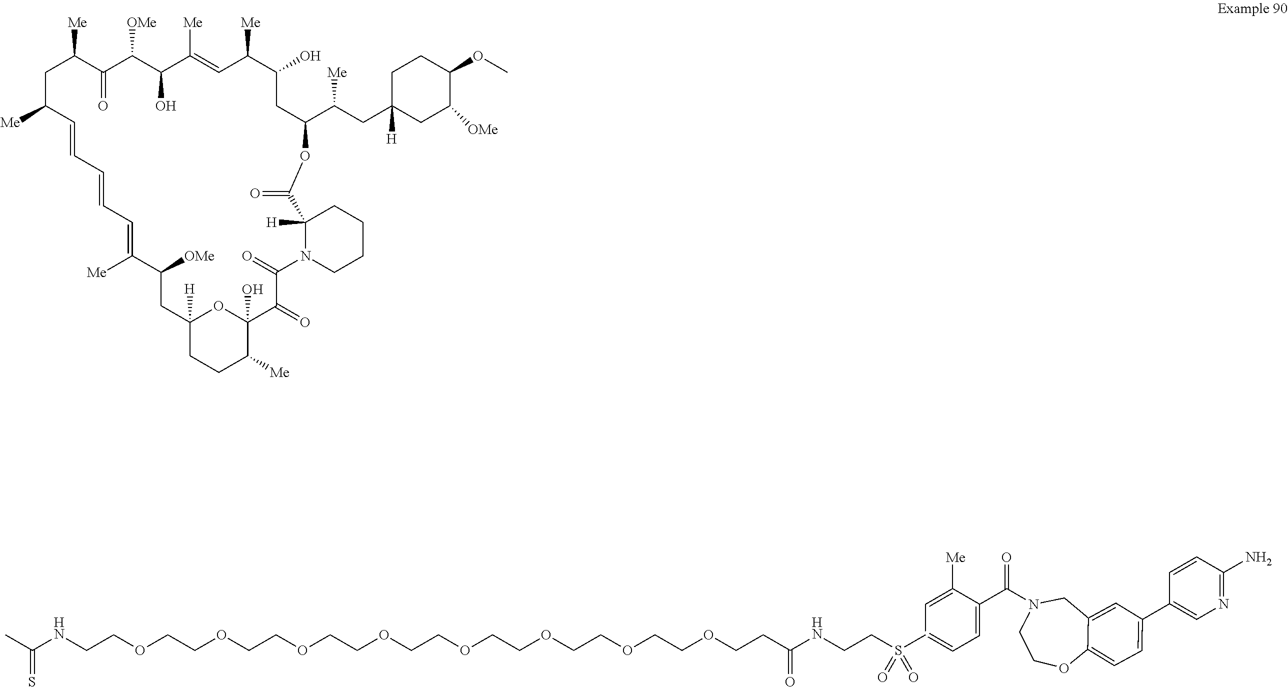

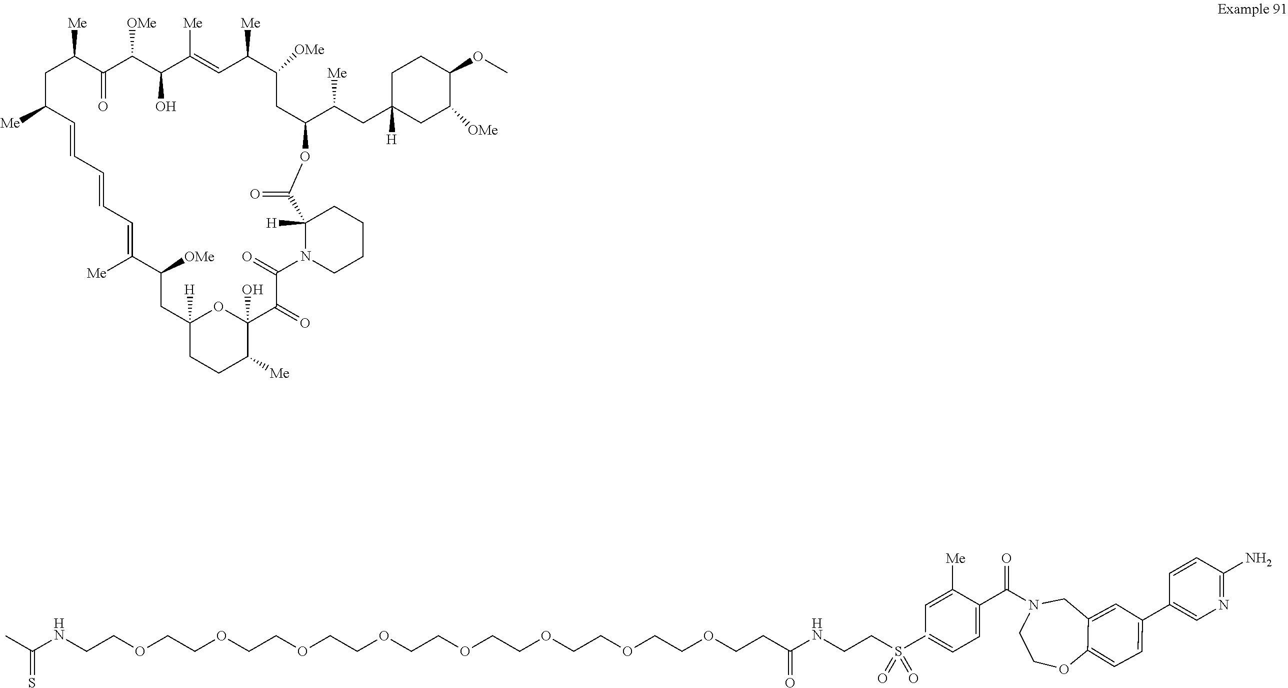

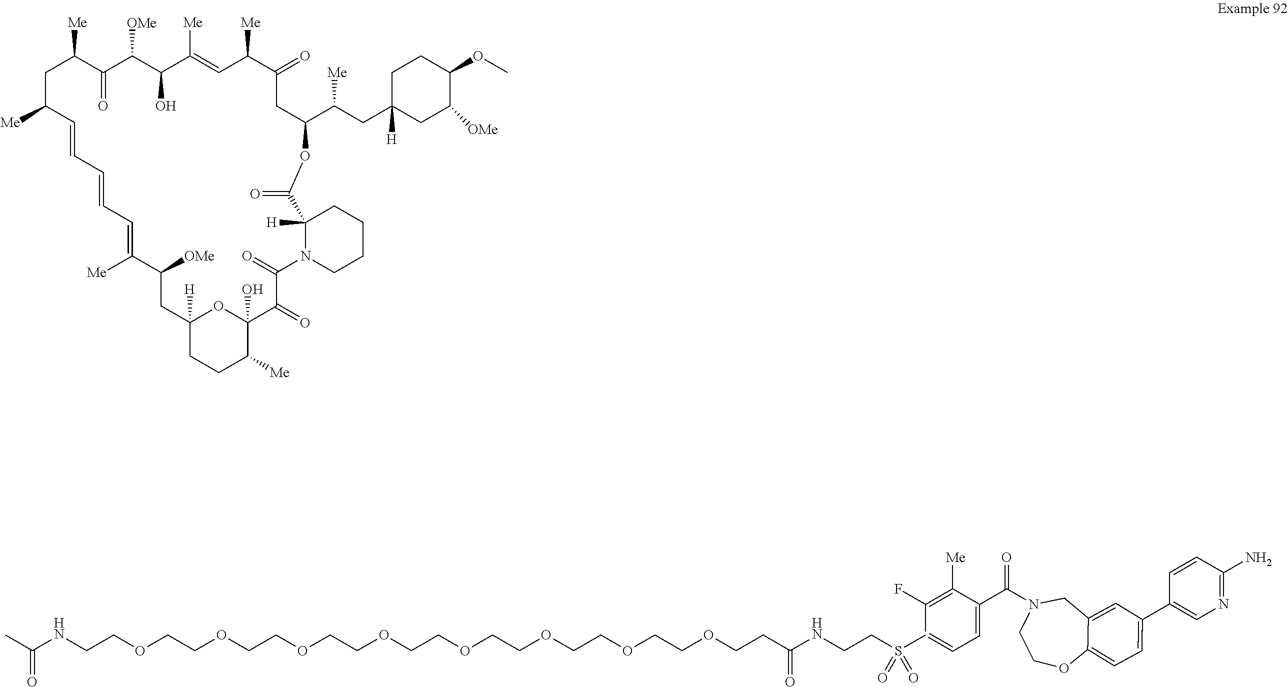

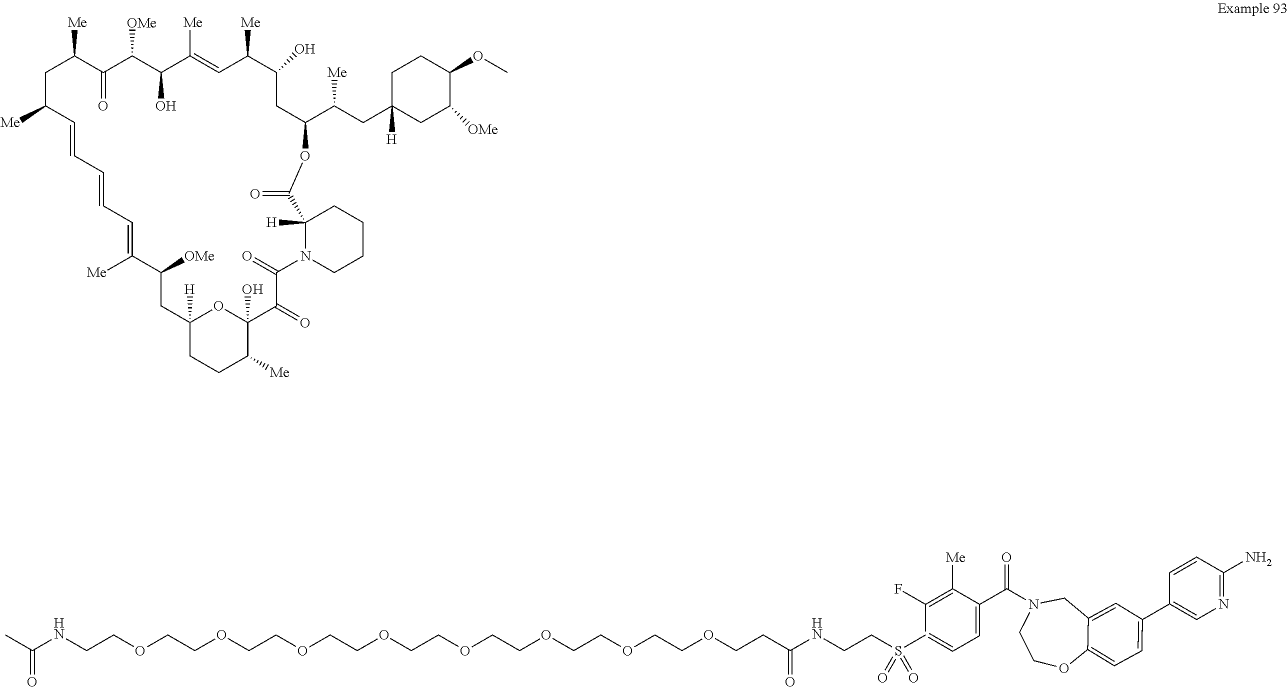

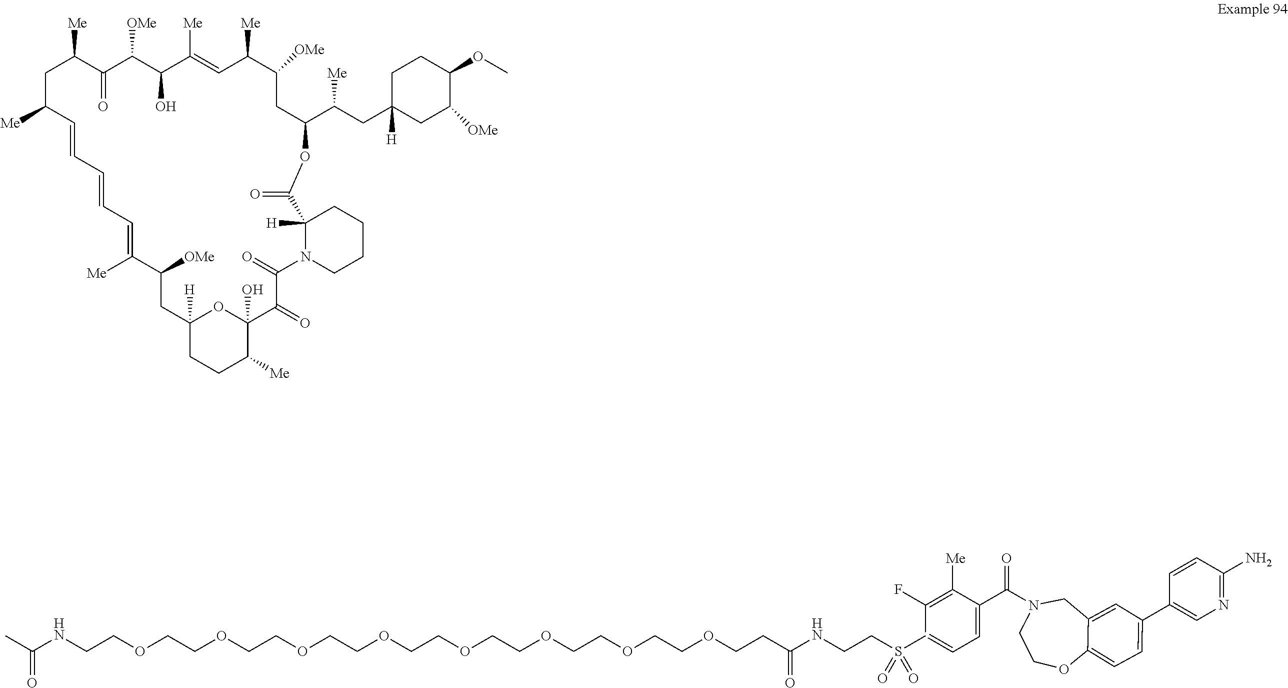

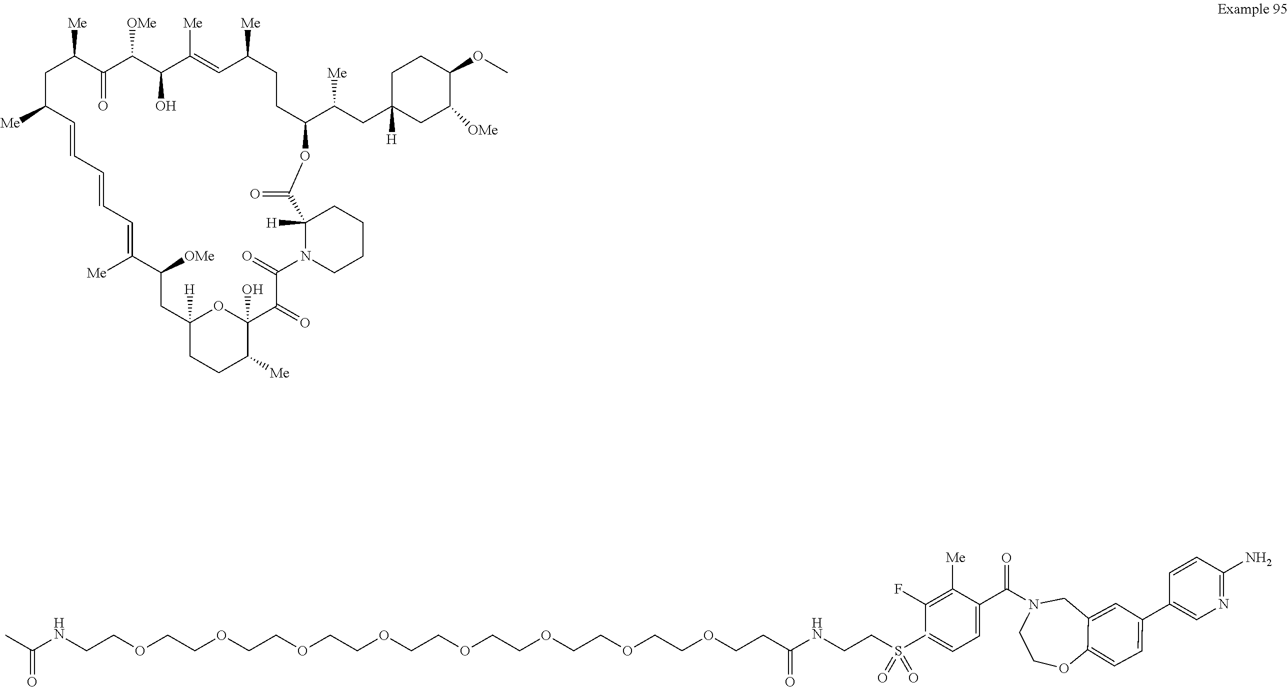

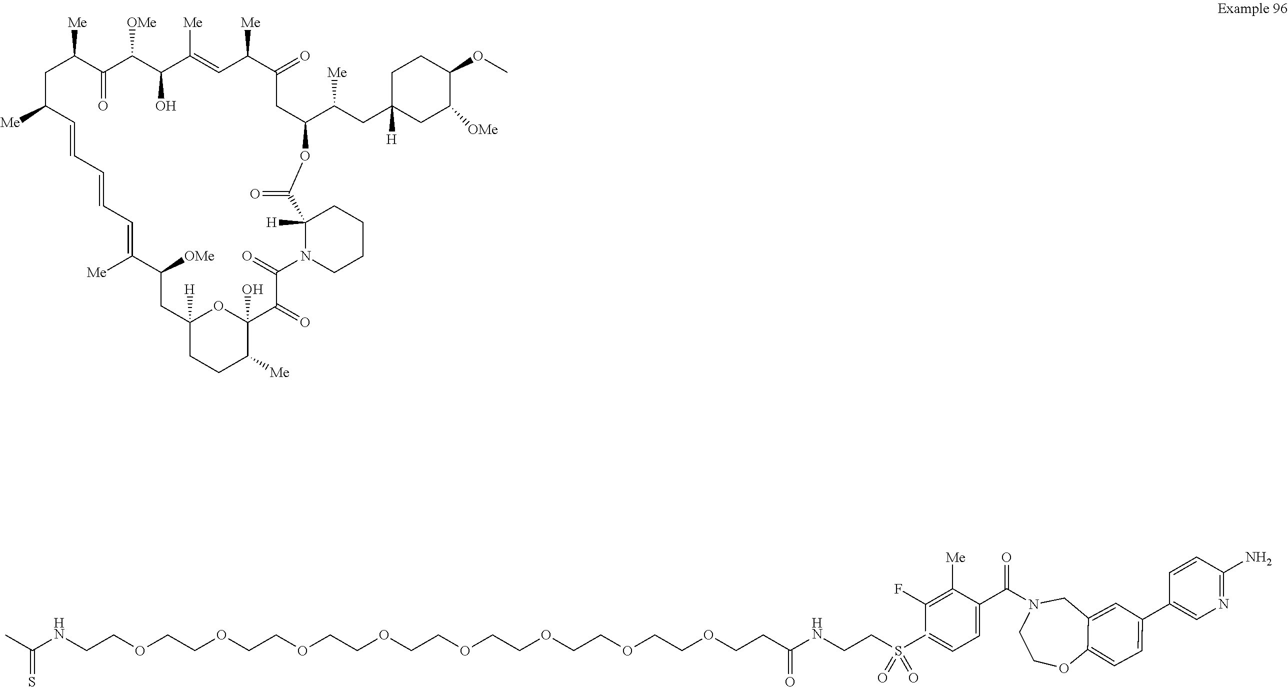

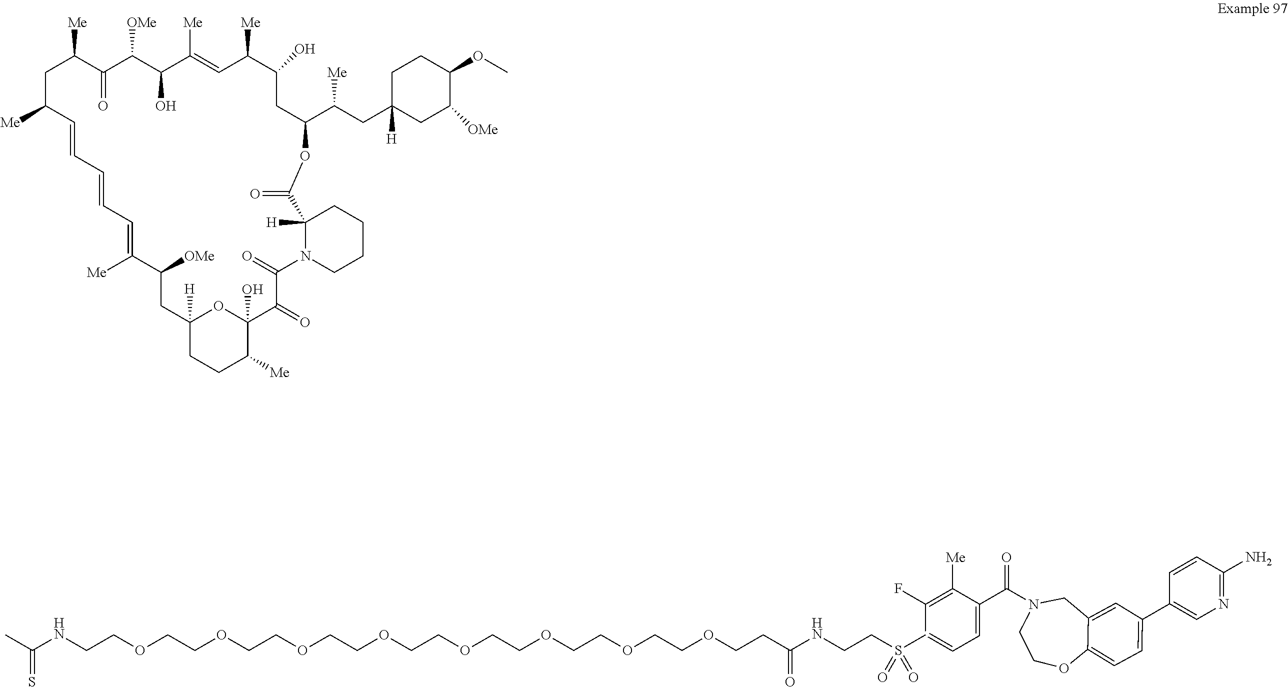

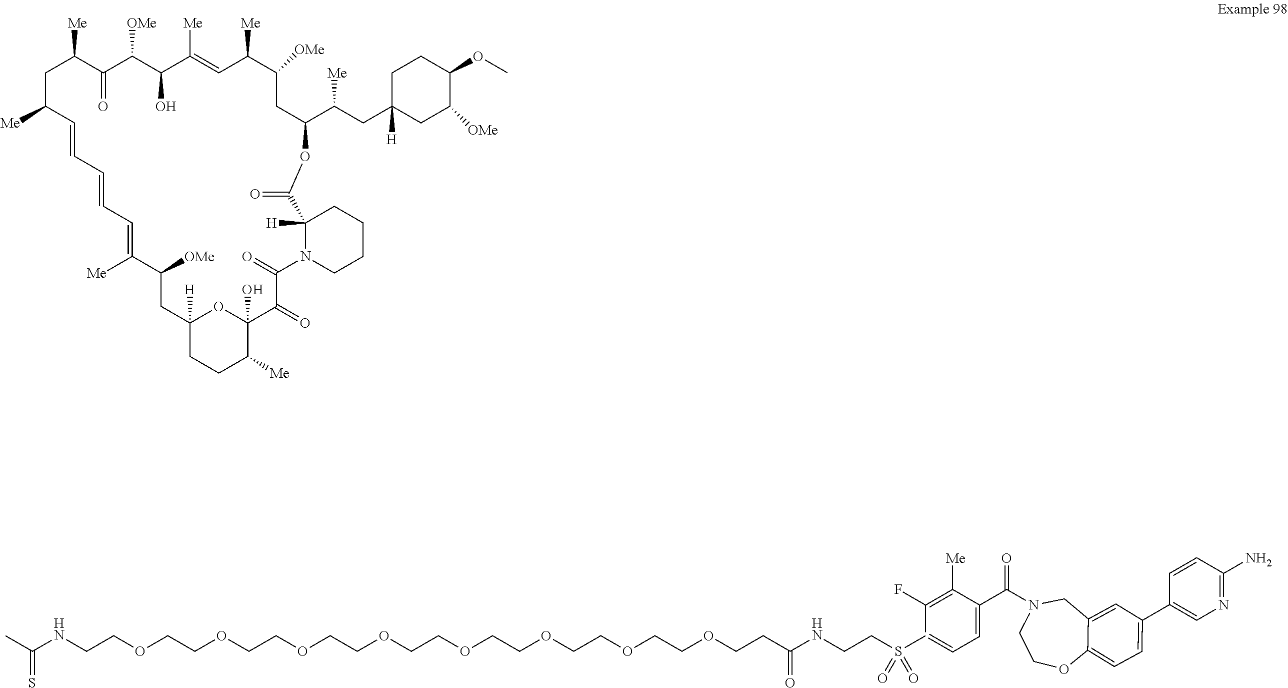

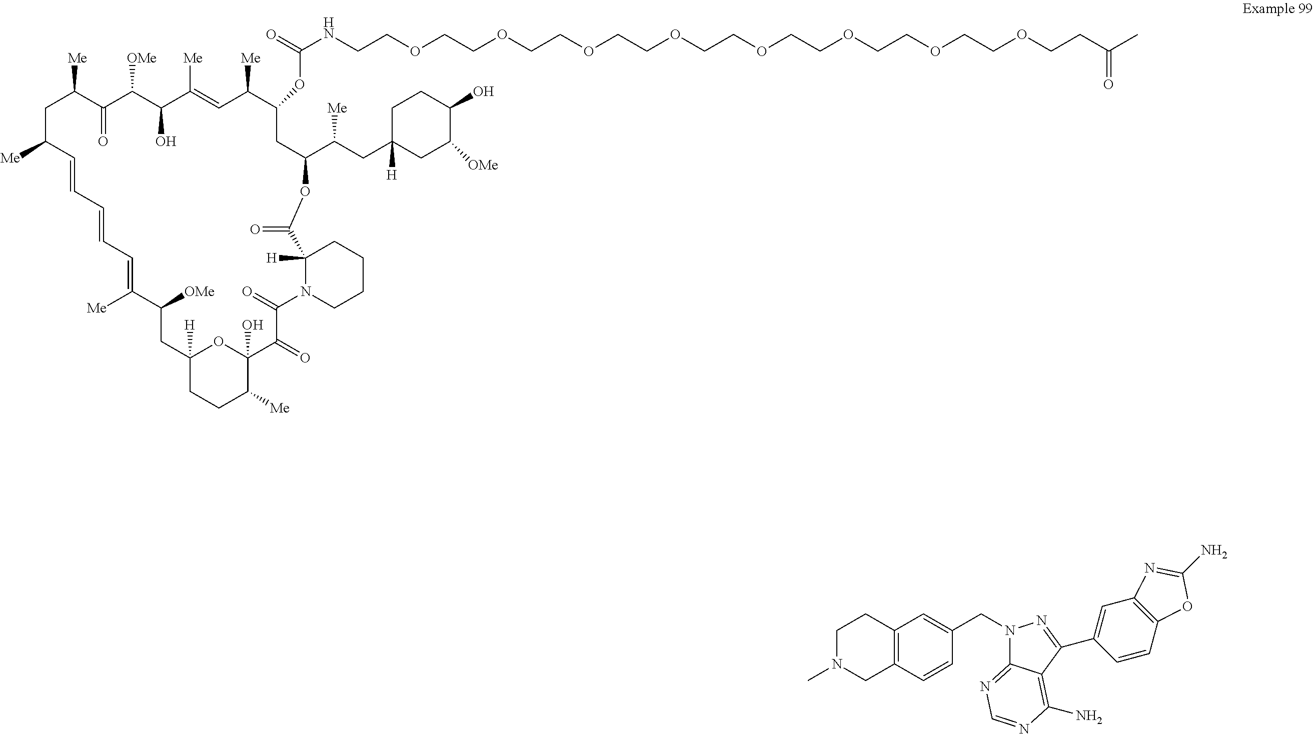

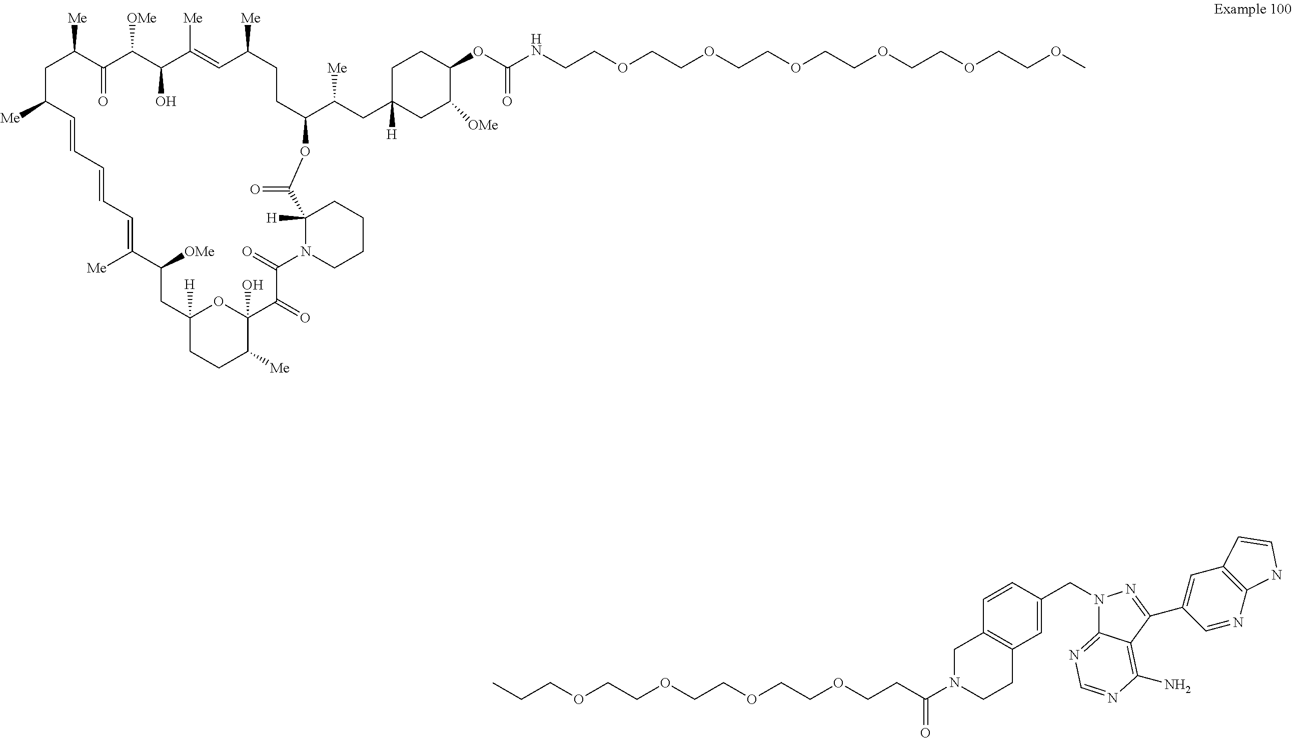

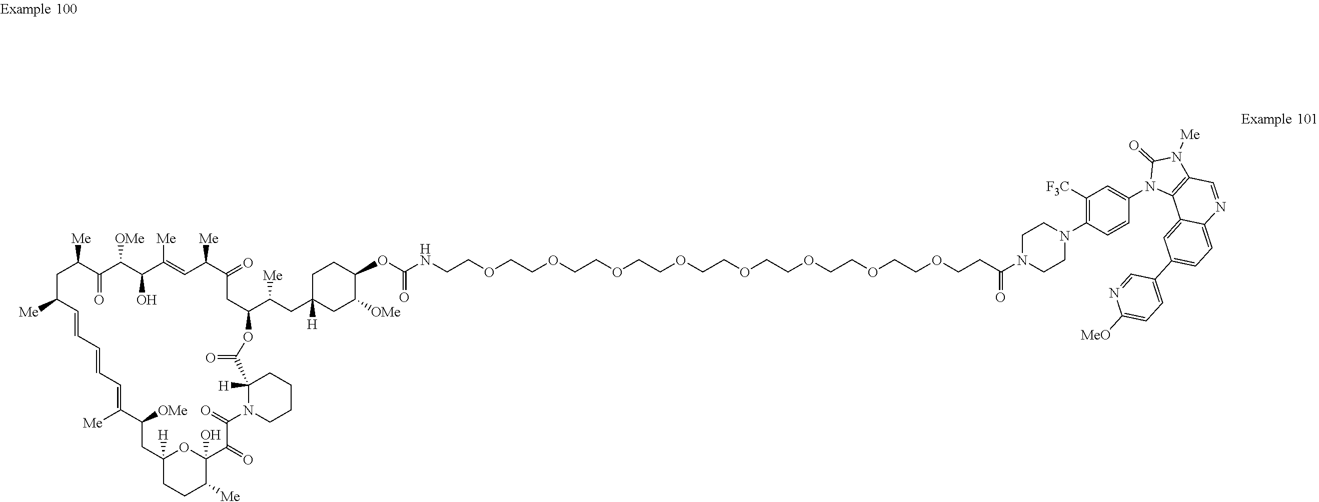

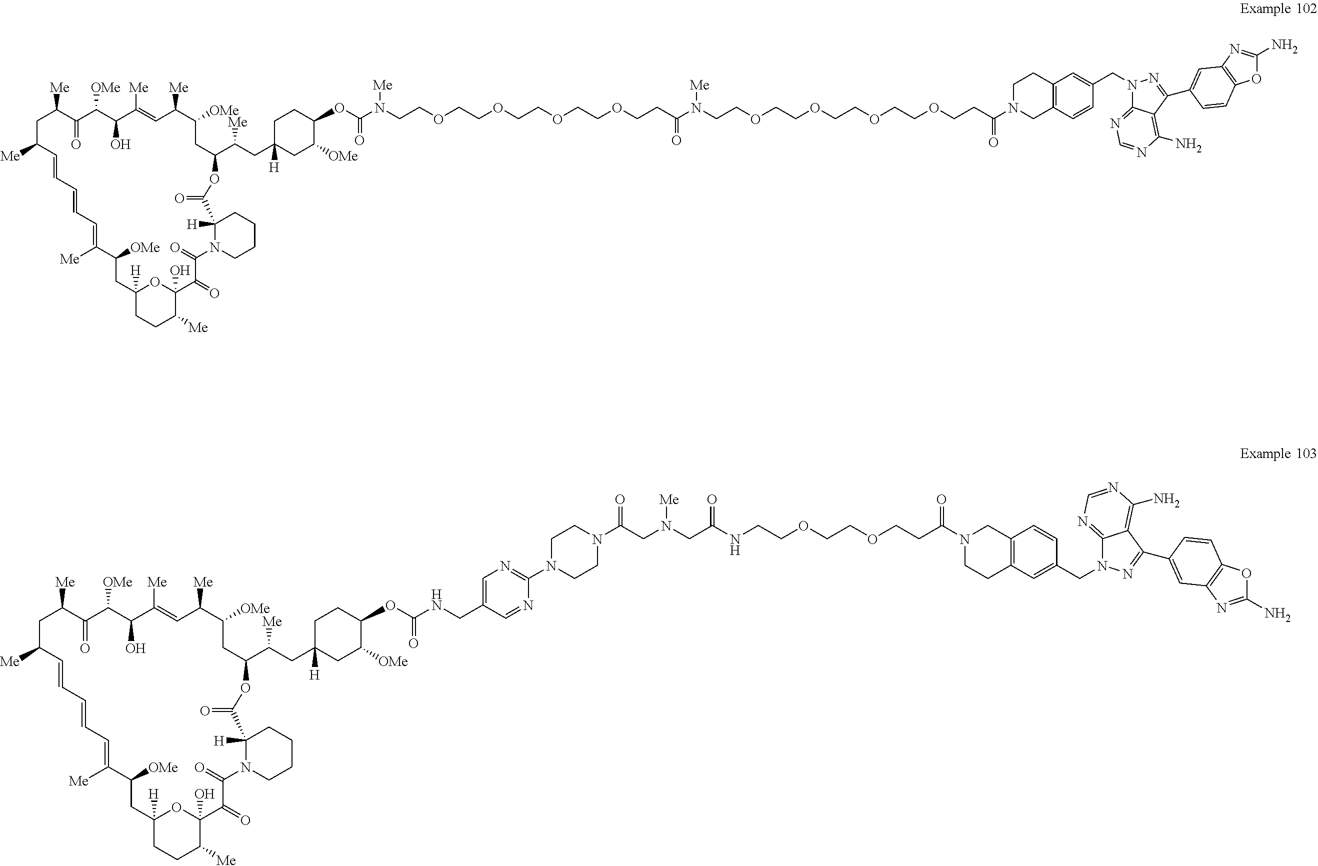

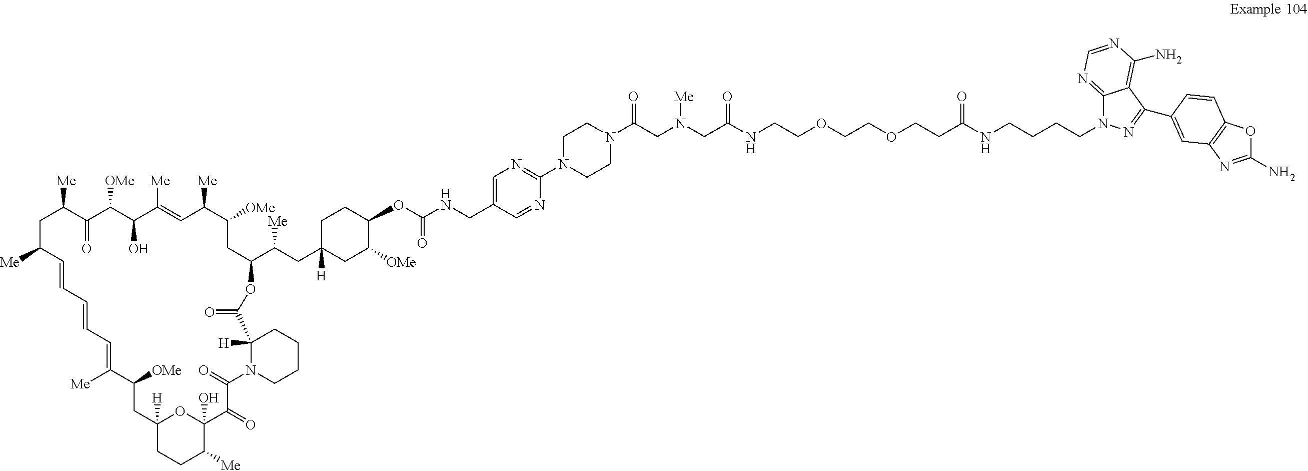

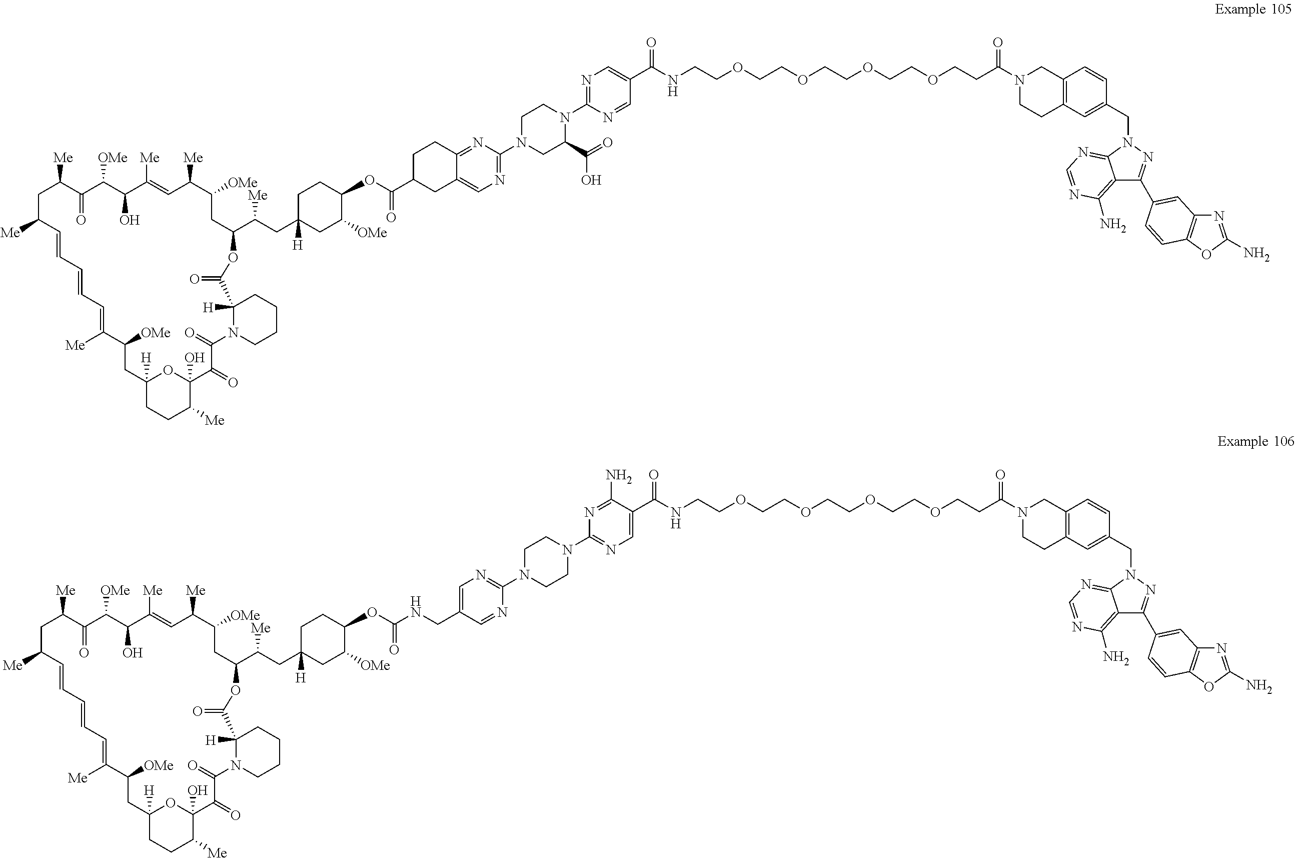

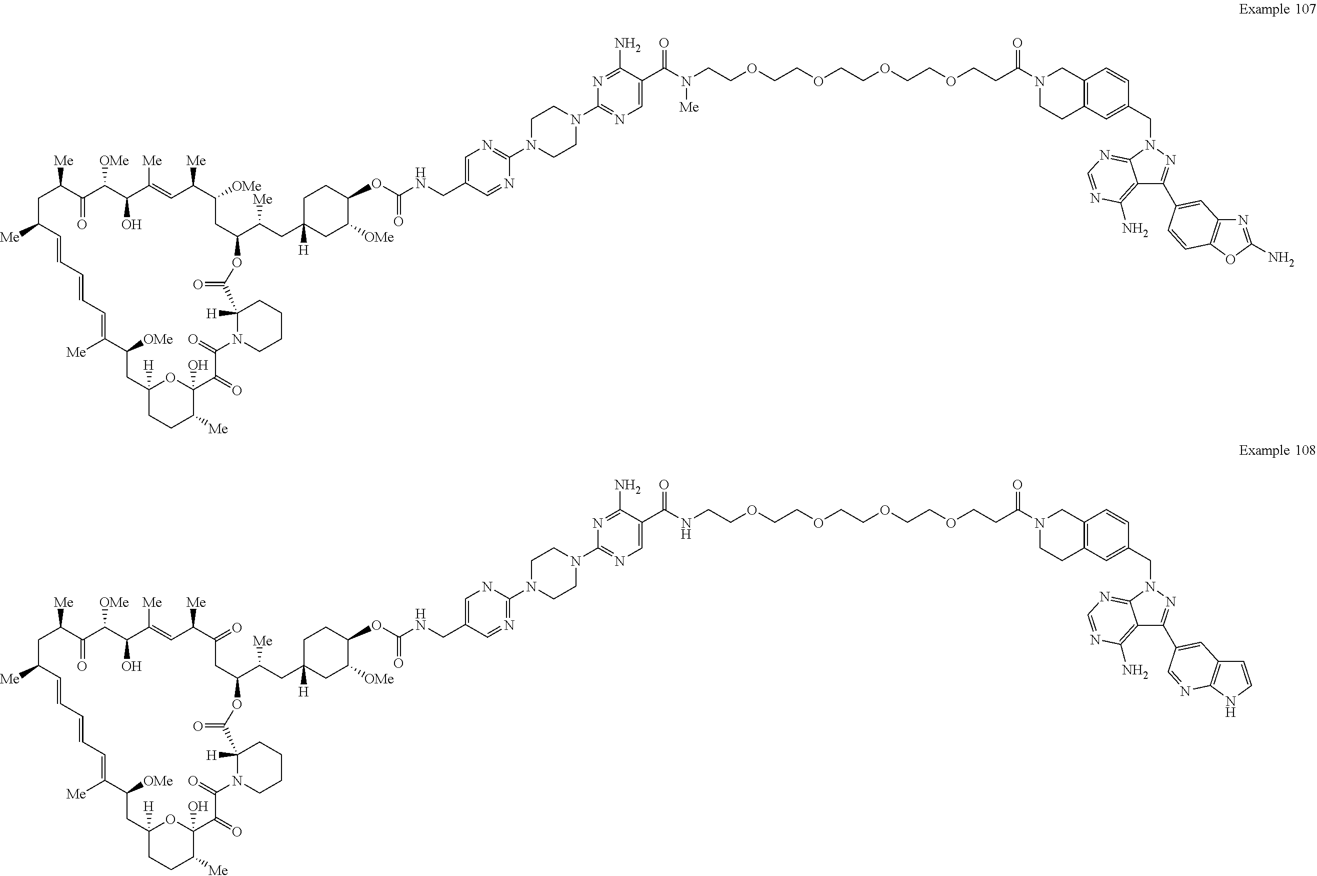

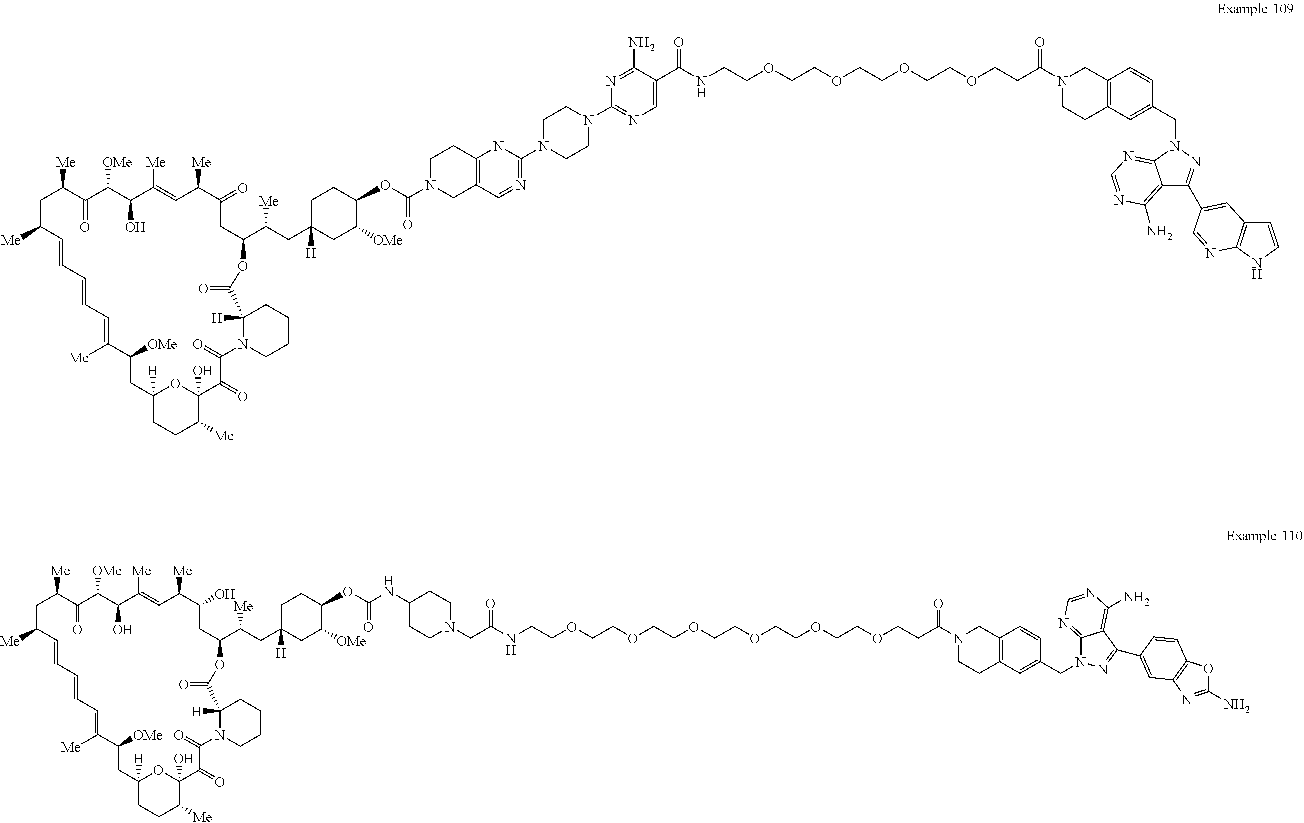

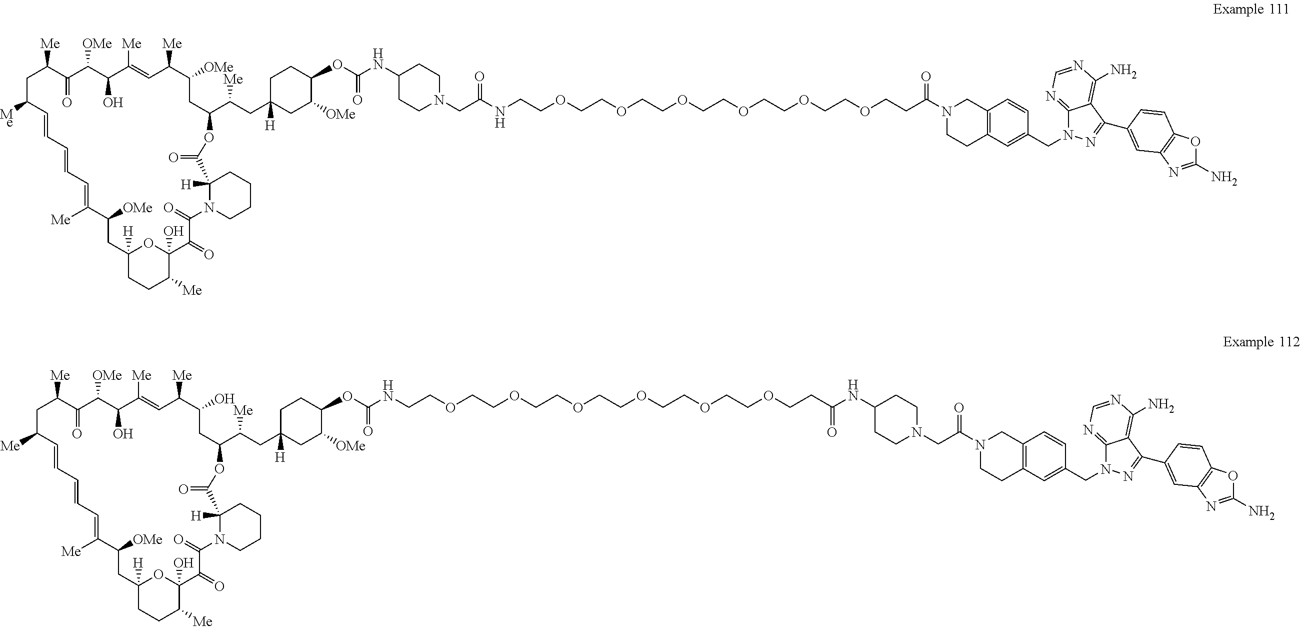

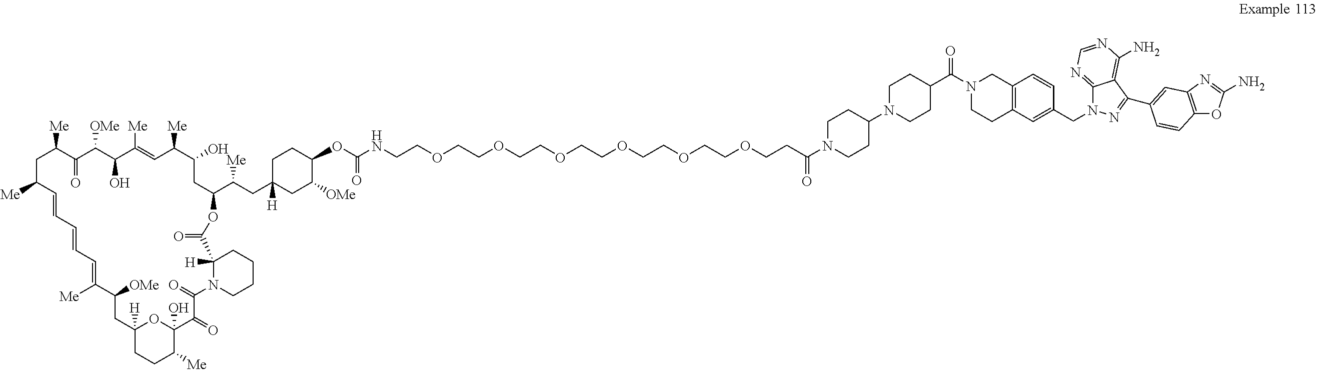

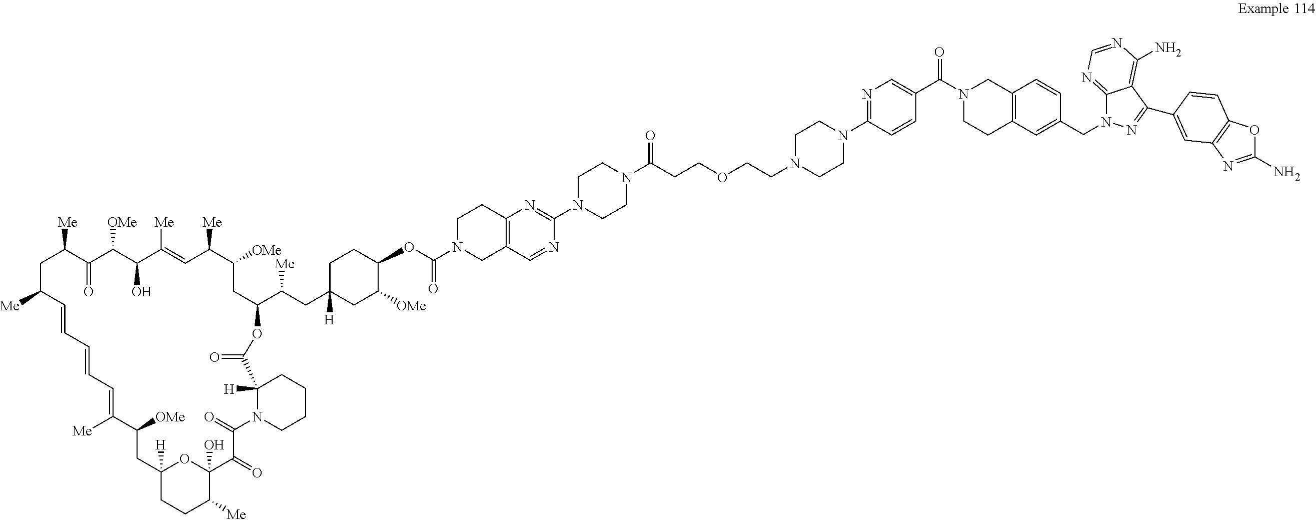

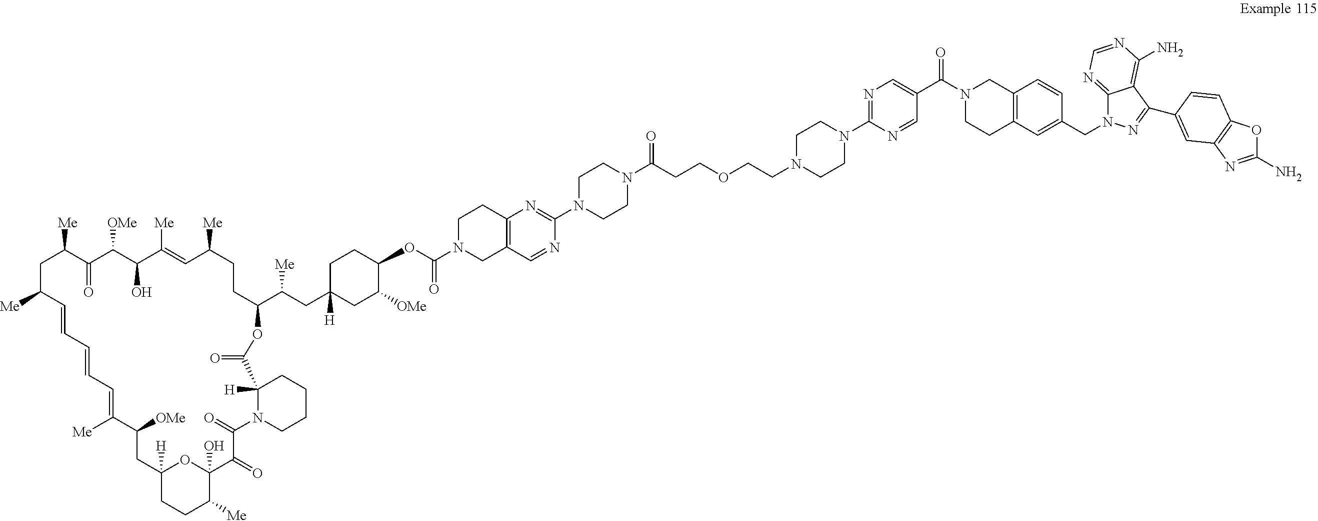

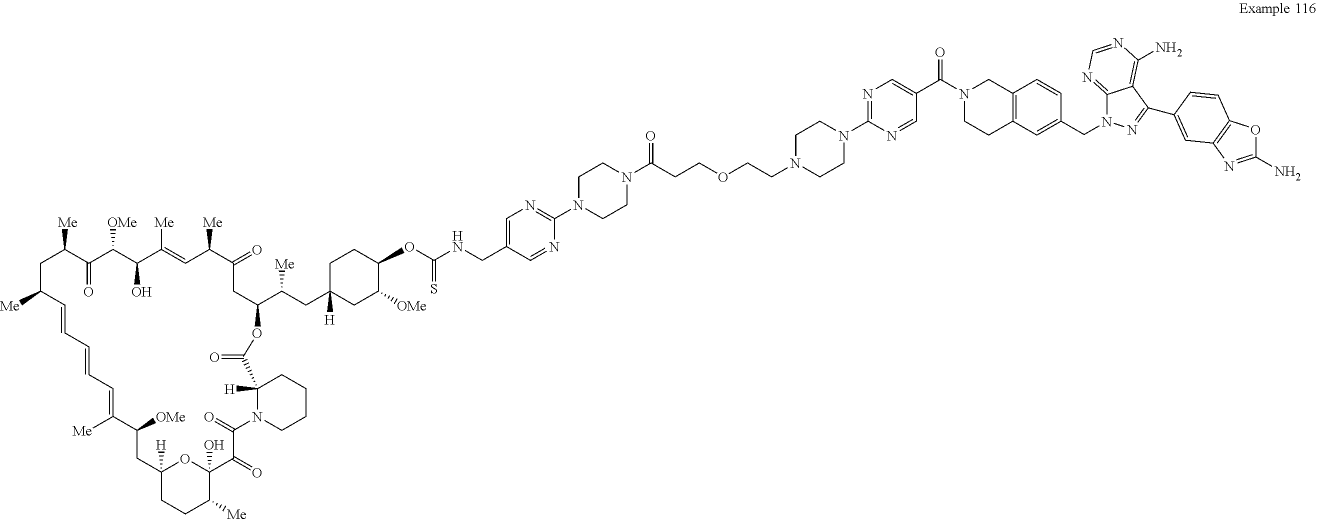

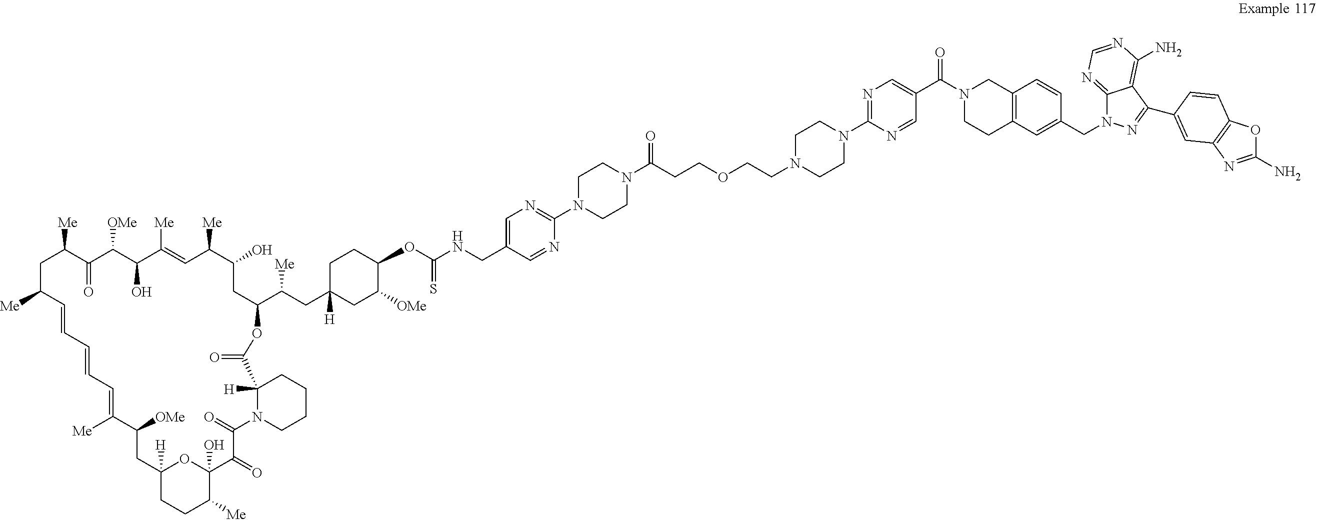

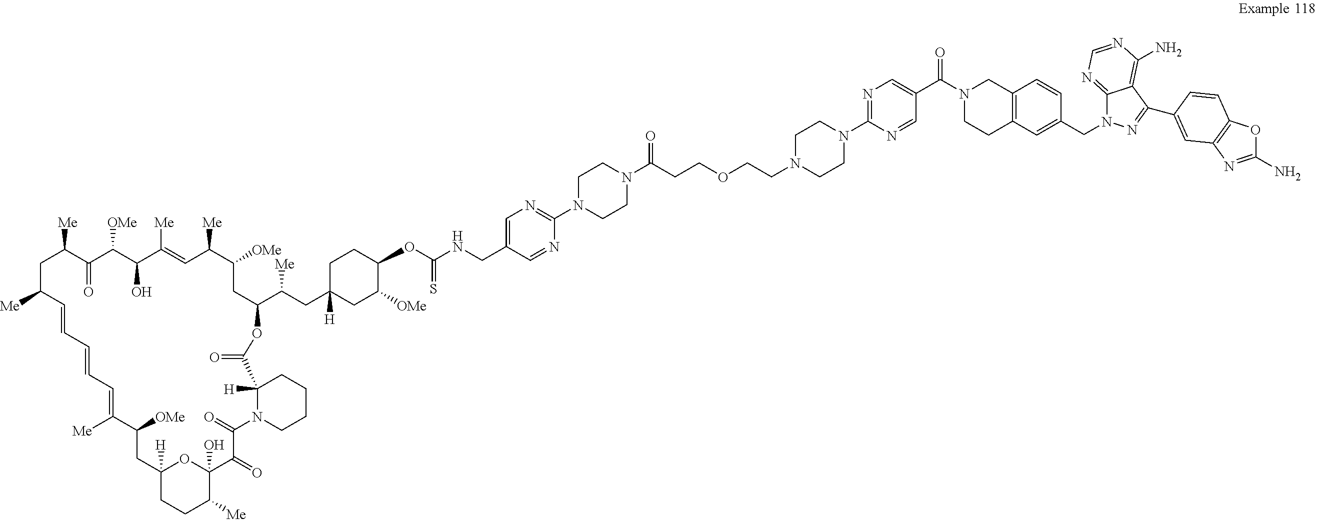

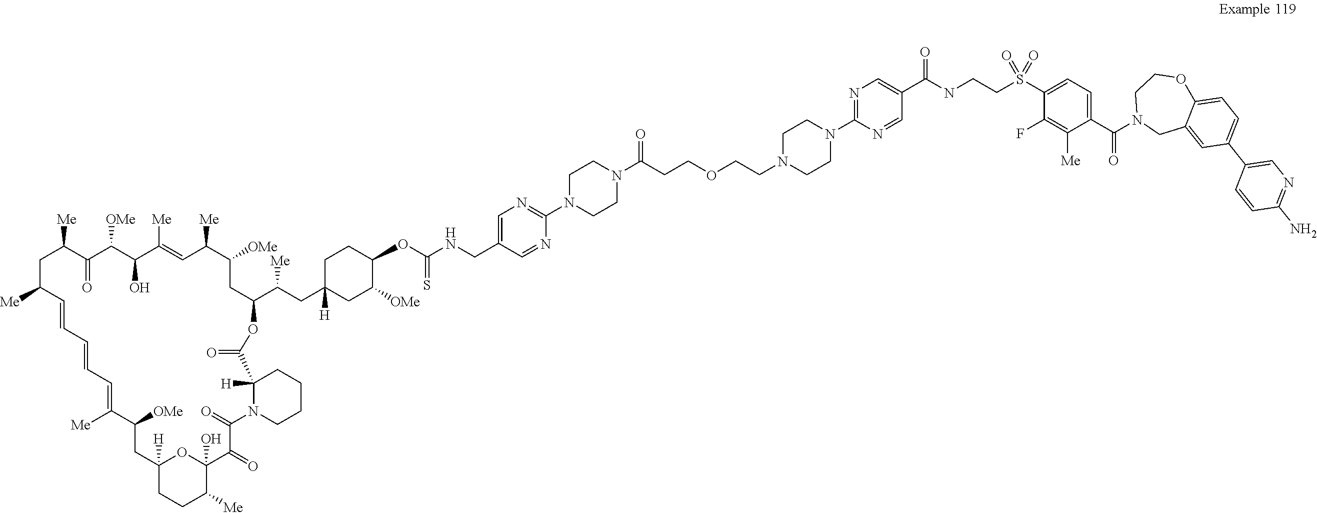

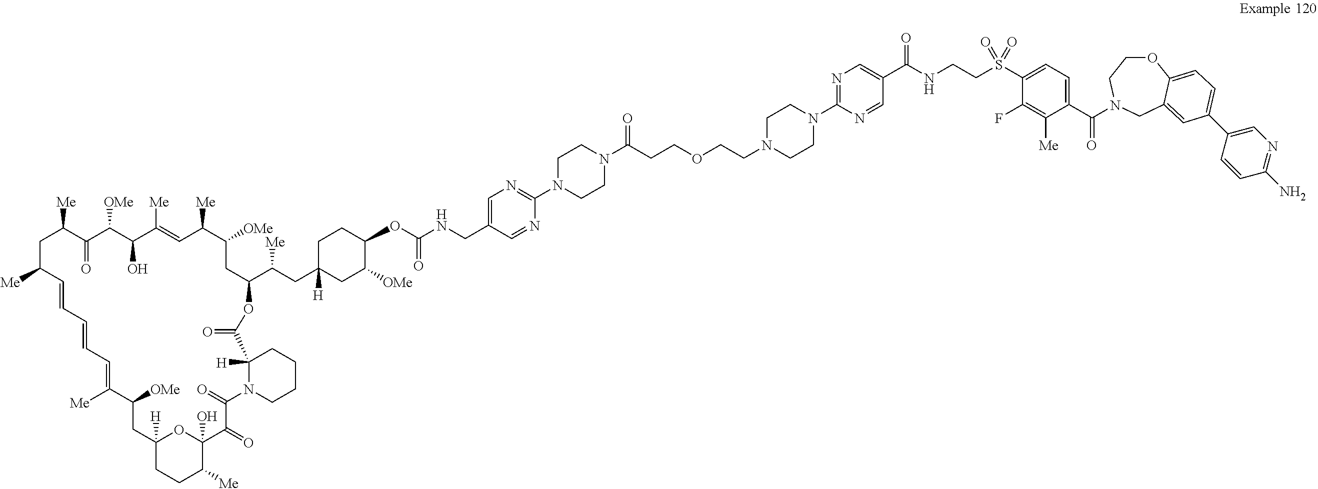

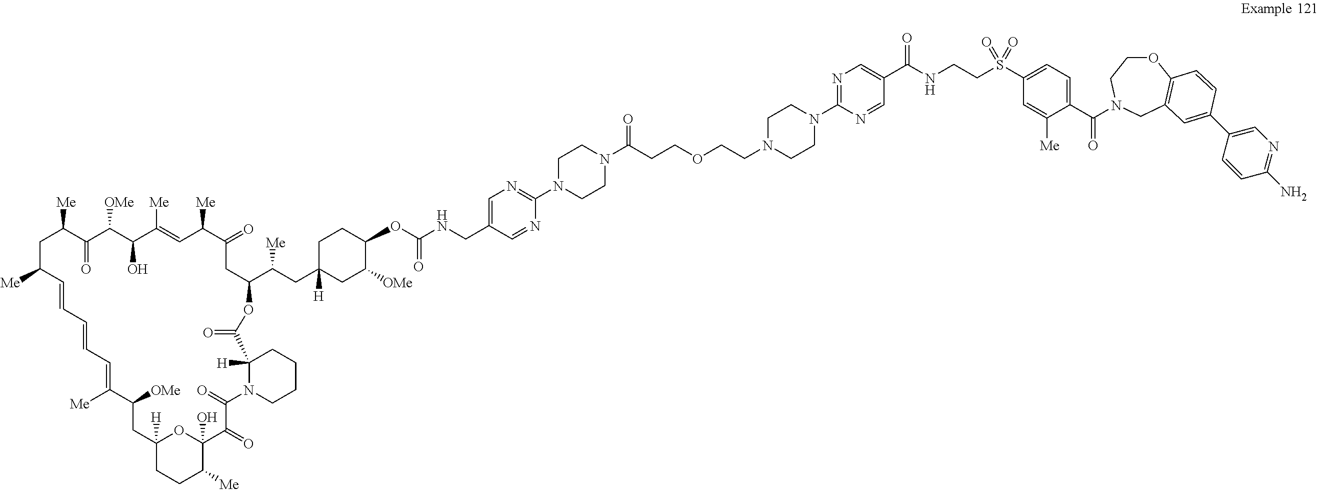

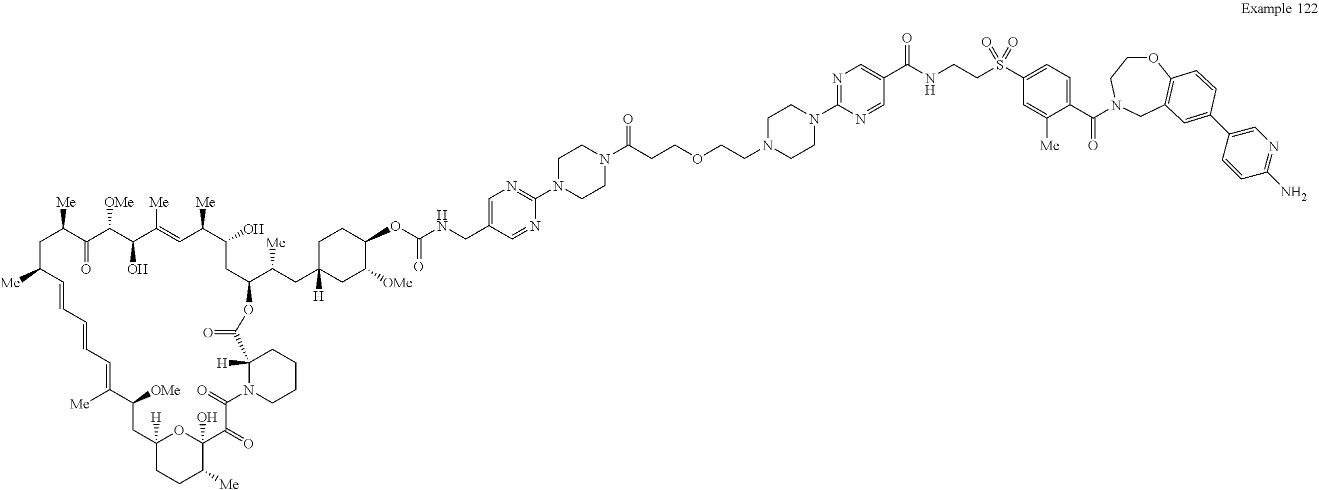

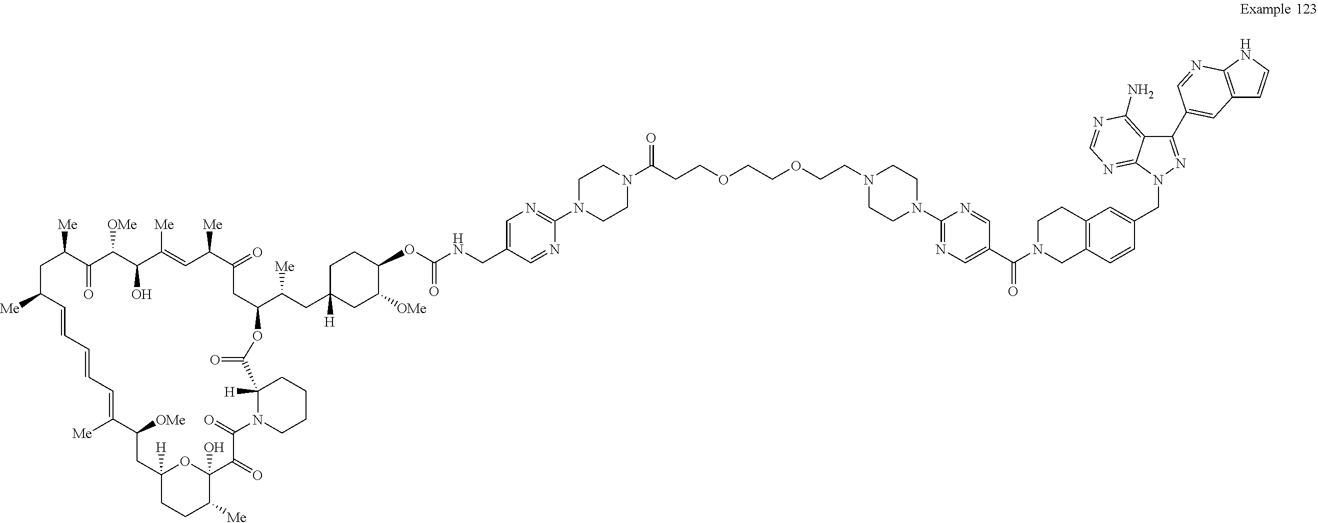

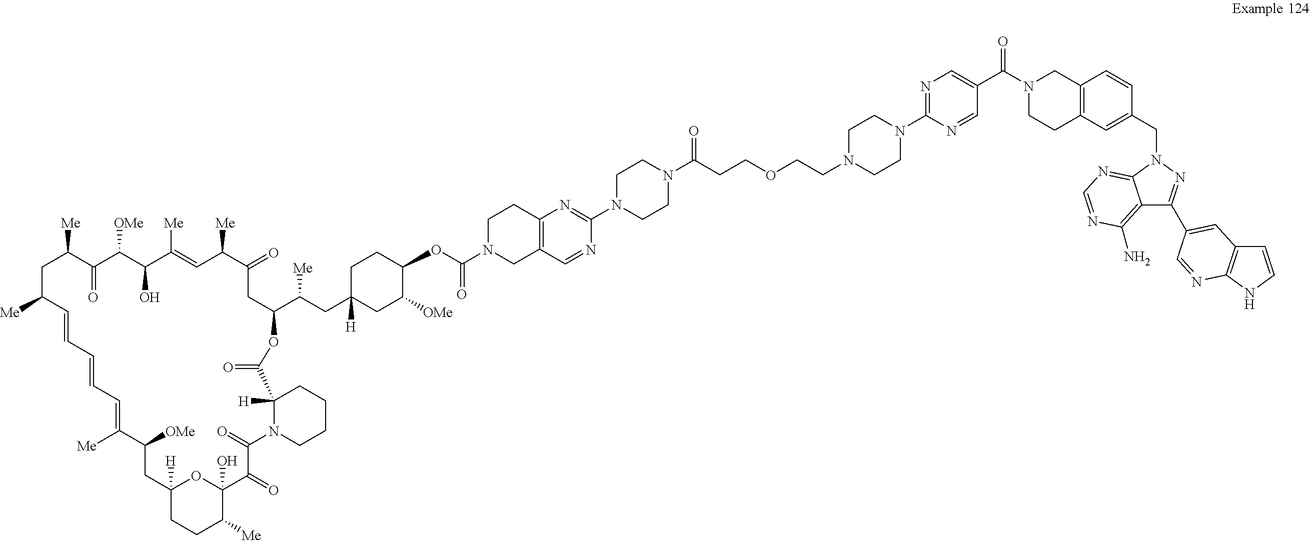

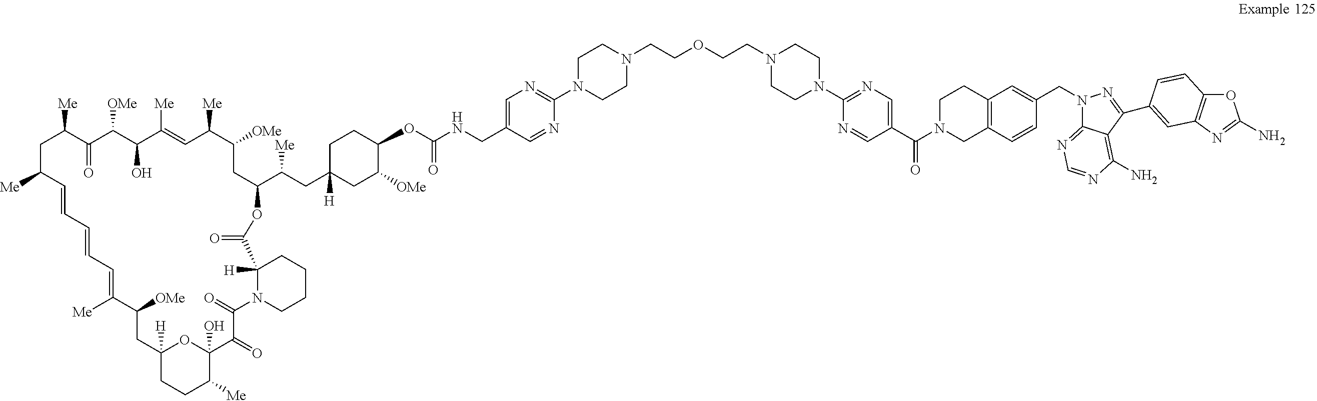

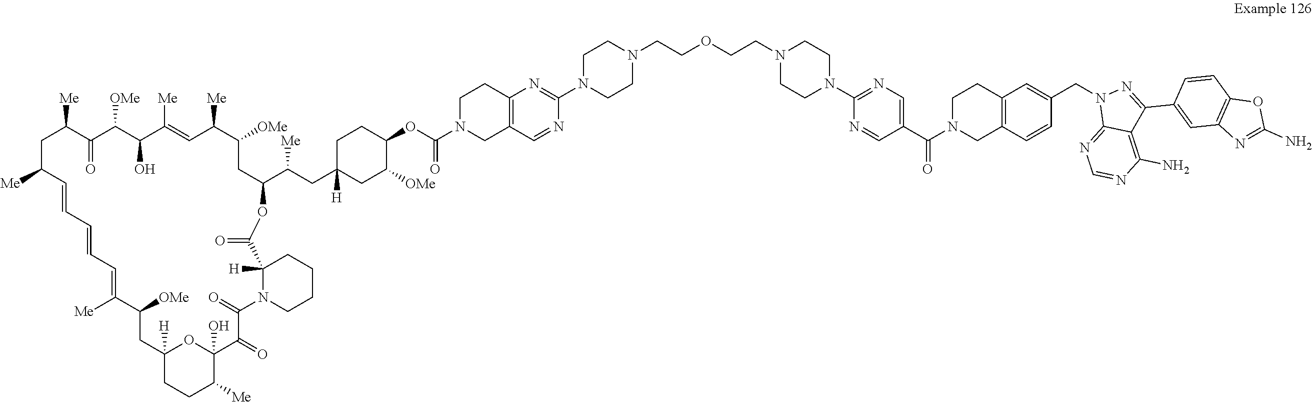 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCN6CCN(c7ncc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

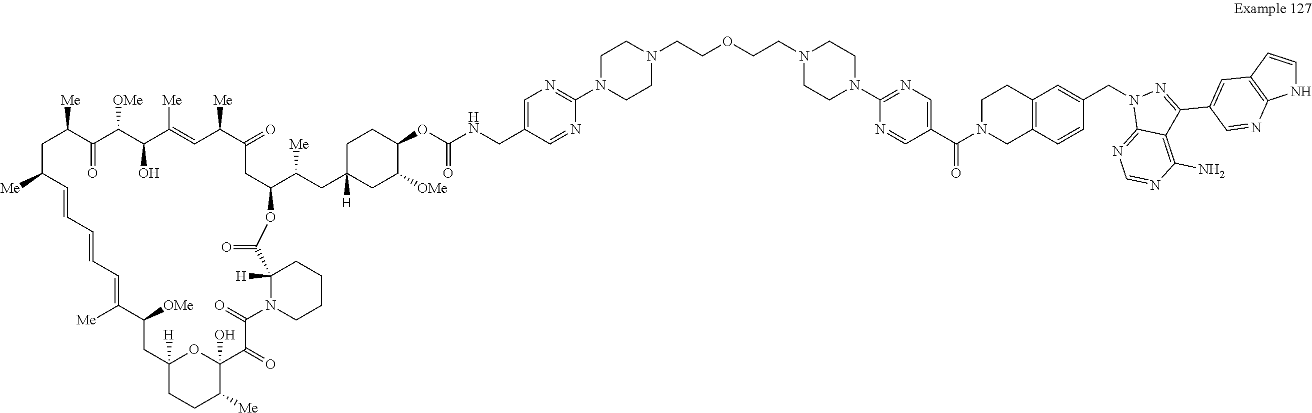 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

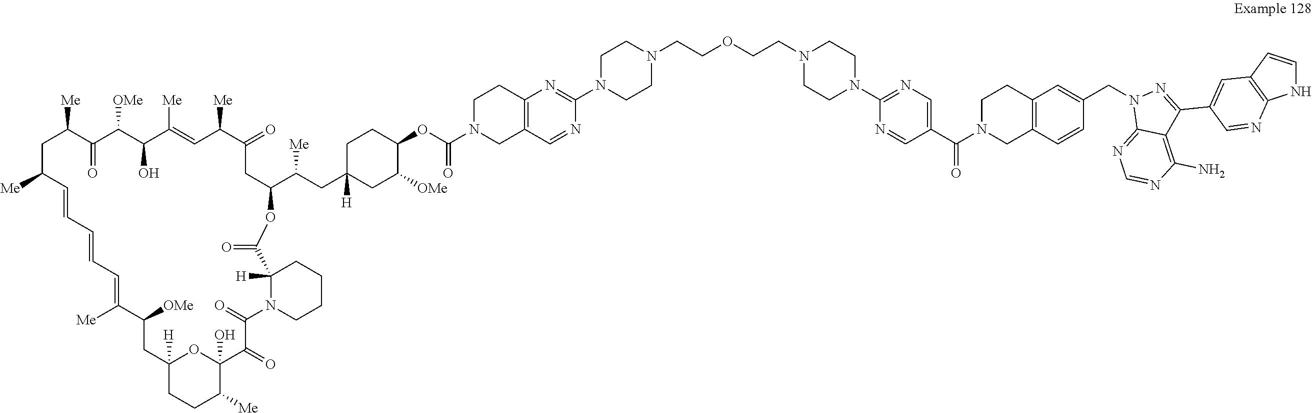 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCN6CCN(c7ncc(C(=O)N8CCc9cc(Cn%10nc(-c%11cnc%12[nH]ccc%12c%11)c%11c(N)ncnc%11%10)ccc9C8)cn7)CC6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

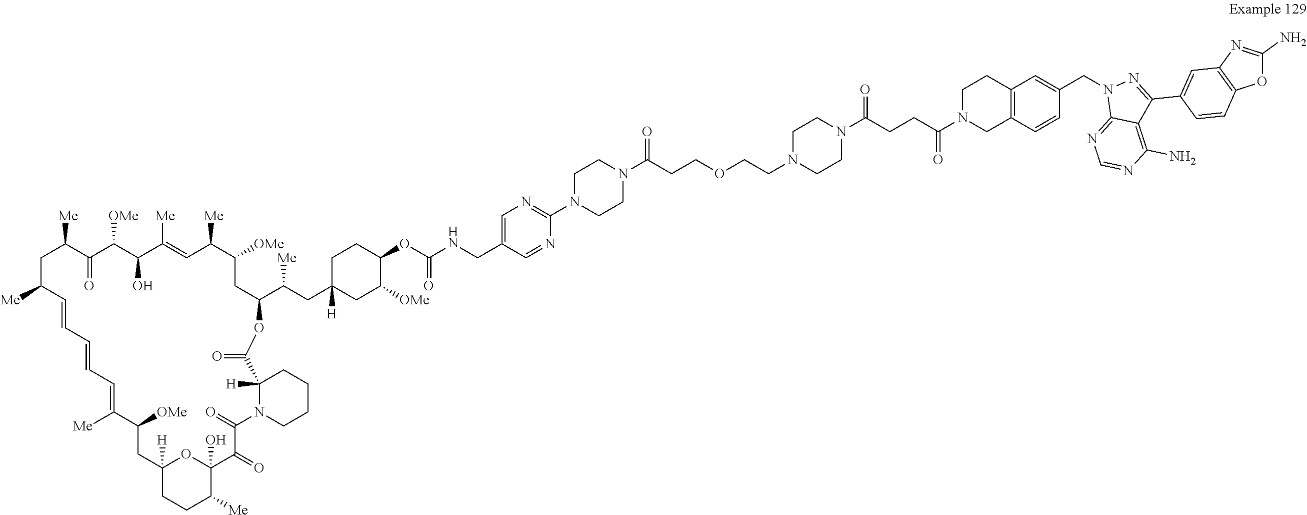 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)N6CCc7cc(Cn8nc(-c9ccc%10oc(N)nc%10c9)c9c(N)ncnc98)ccc7C6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

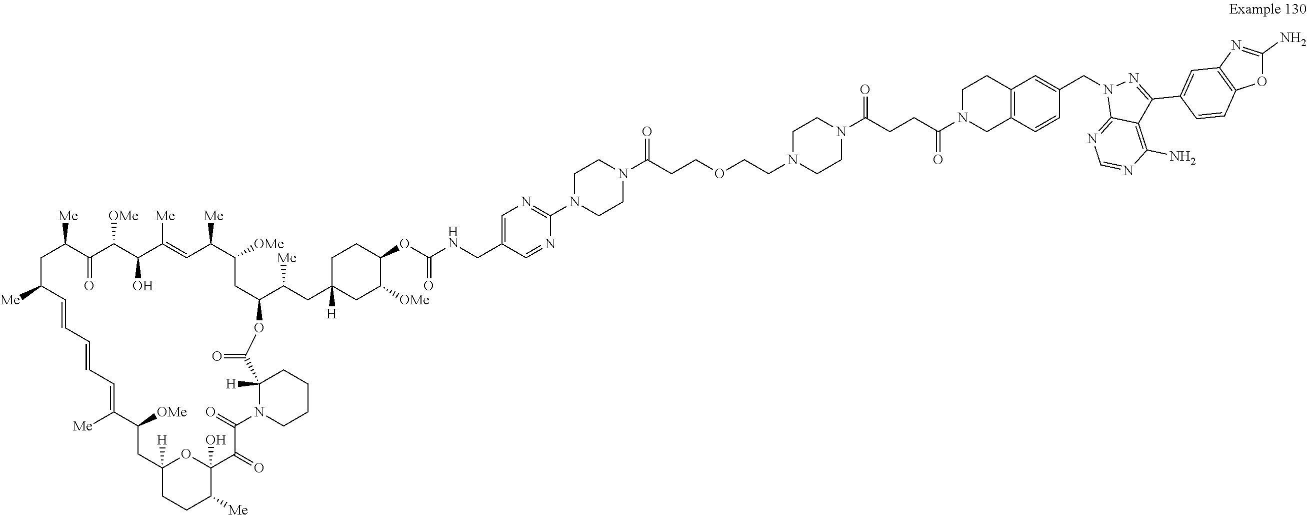 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)N6CCc7cc(Cn8nc(-c9ccc%10oc(N)nc%10c9)c9c(N)ncnc98)ccc7C6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

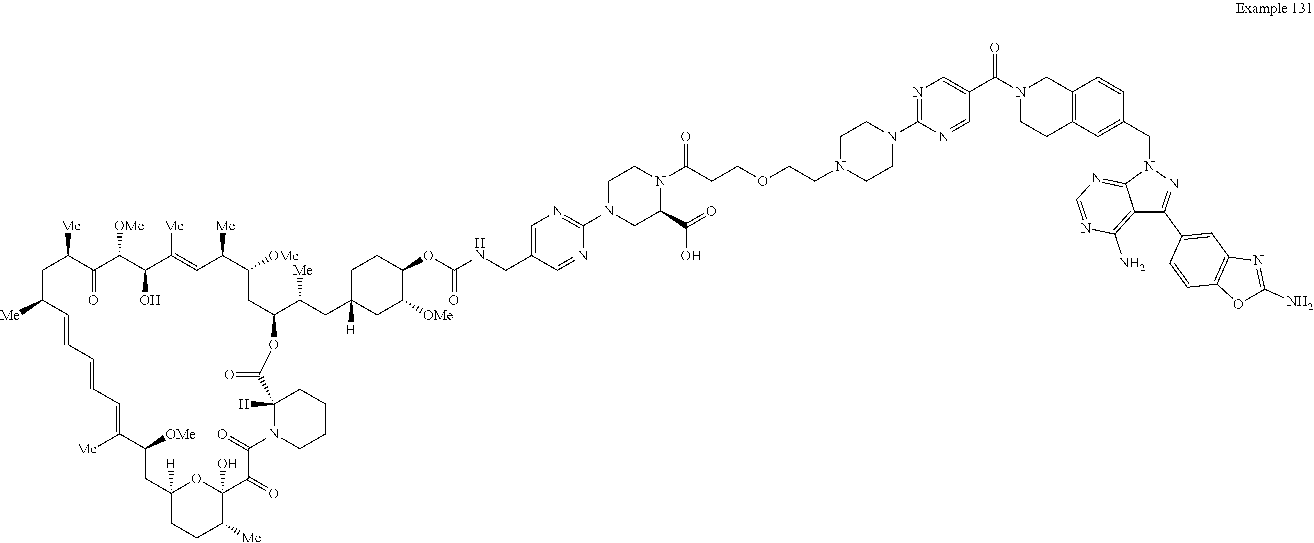 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)[C@@H](C(=O)O)C4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

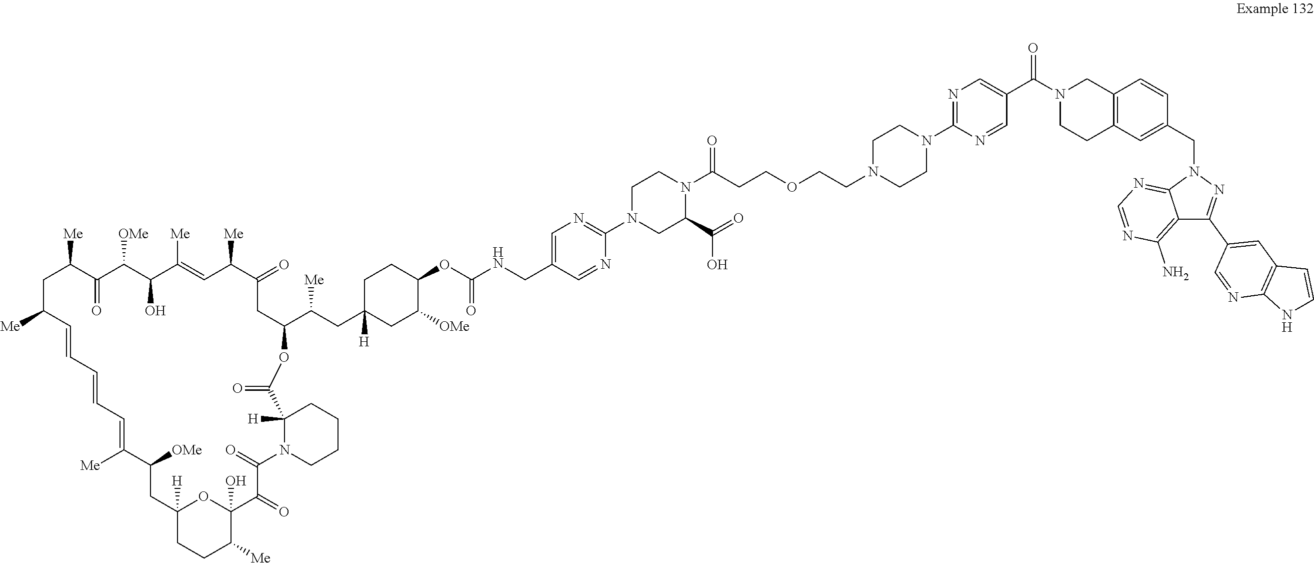 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)[C@@H](C(=O)O)C4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

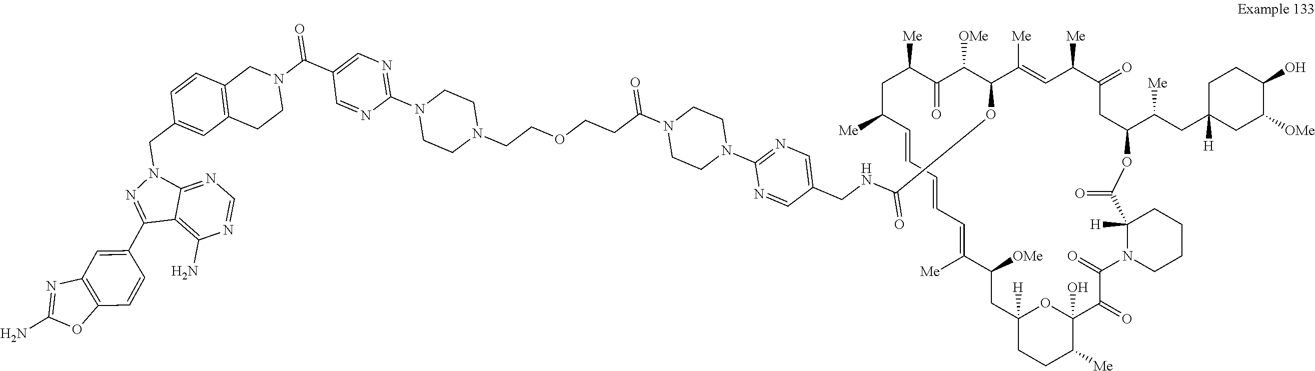 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](OC(=O)NCc2cnc(N3CCN(C(=O)CCOCCN4CCN(c5ncc(C(=O)N6CCc7cc(Cn8nc(-c9ccc%10oc(N)nc%10c9)c9c(N)ncnc98)ccc7C6)cn5)CC4)CC3)nc2)[C@@H](OC)C(=O)[C@H](C)C[C@H](C)/C=C/C=C/C=C/1C]

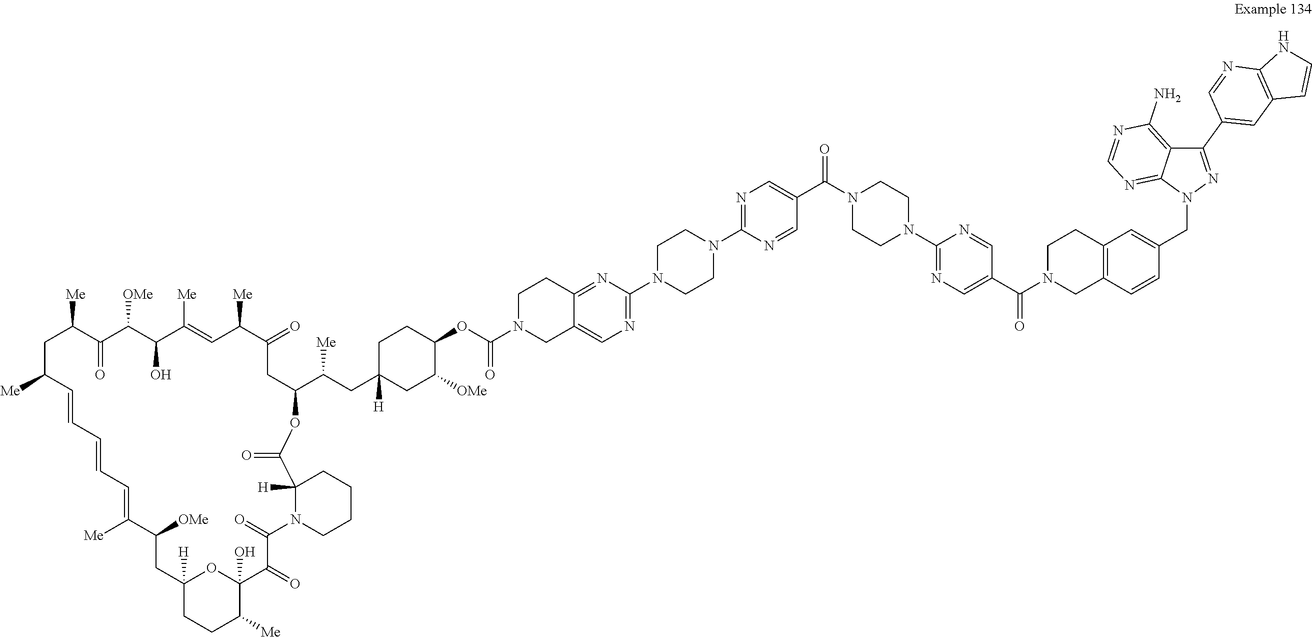 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)N7CCN(c8ncc(C(=O)N9CCc%10cc(Cn%11nc(-c%12cnc%13[nH]ccc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

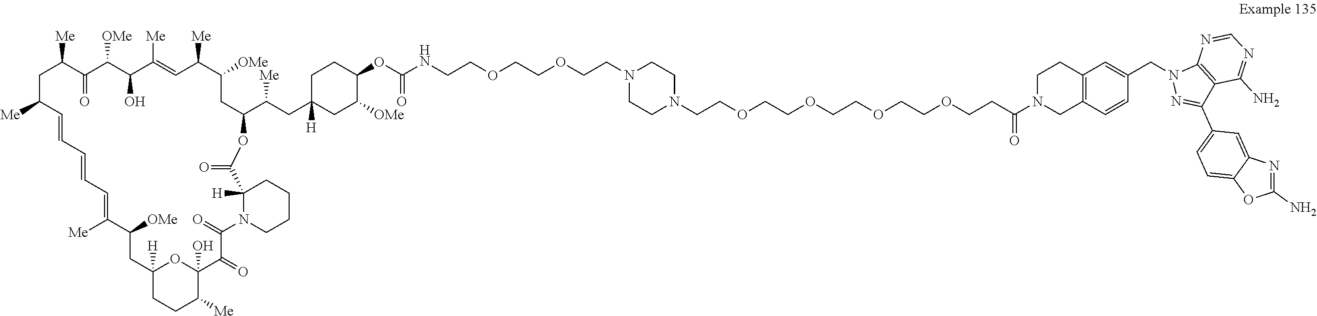 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCN3CCN(CCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

or a pharmaceutically acceptable salt or tautomer thereof.

Embodiment II-74. A compound selected from the group consisting of:

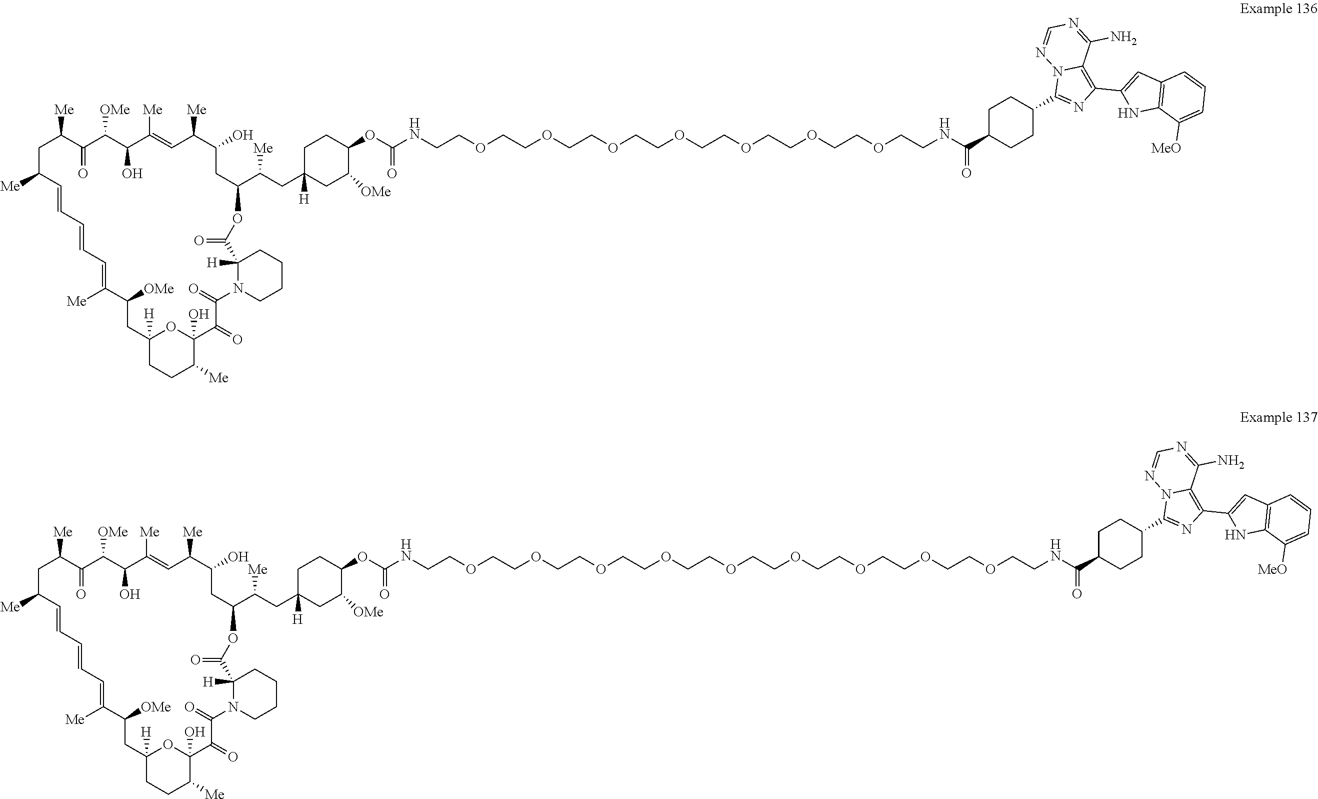

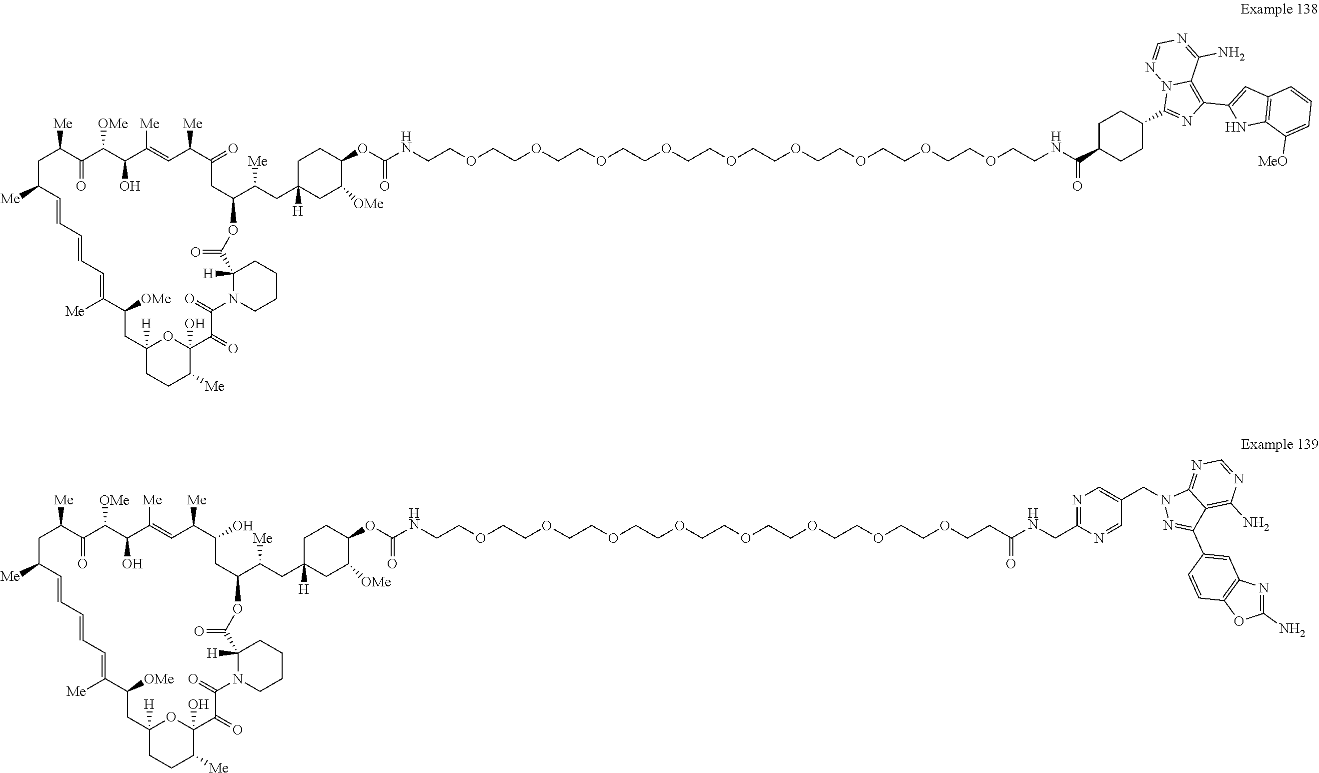

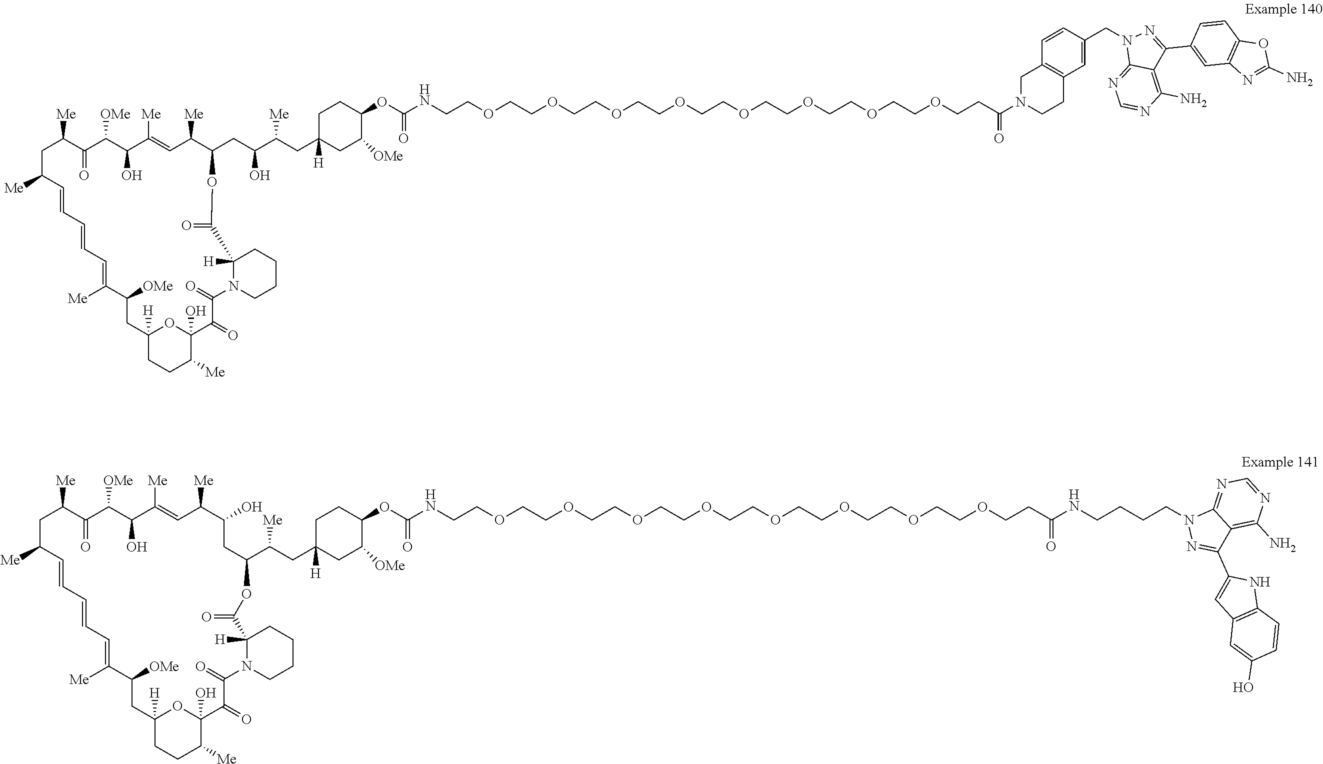

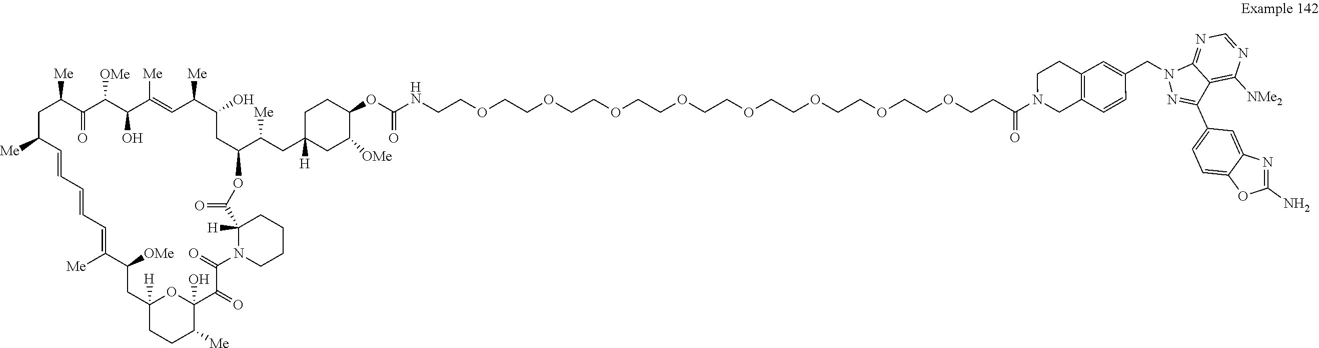 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N(C)C)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

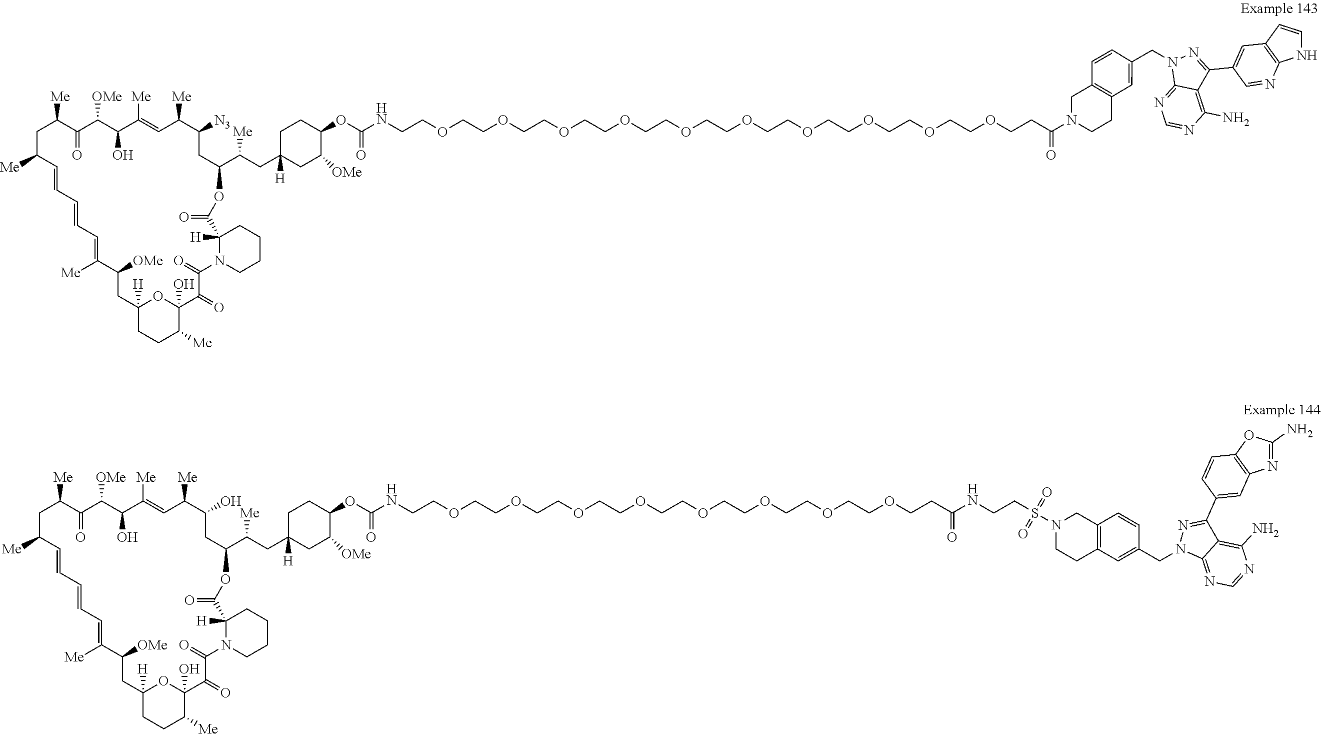

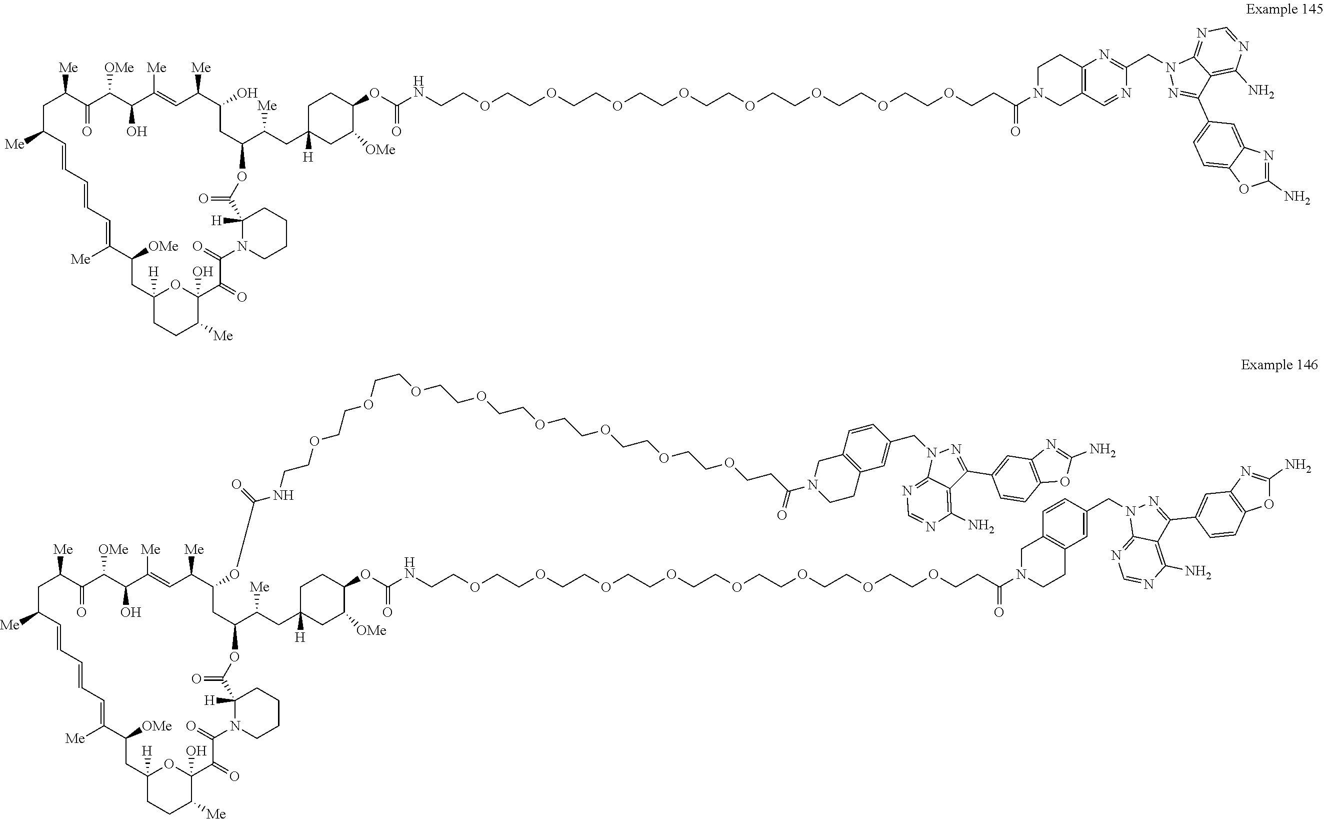

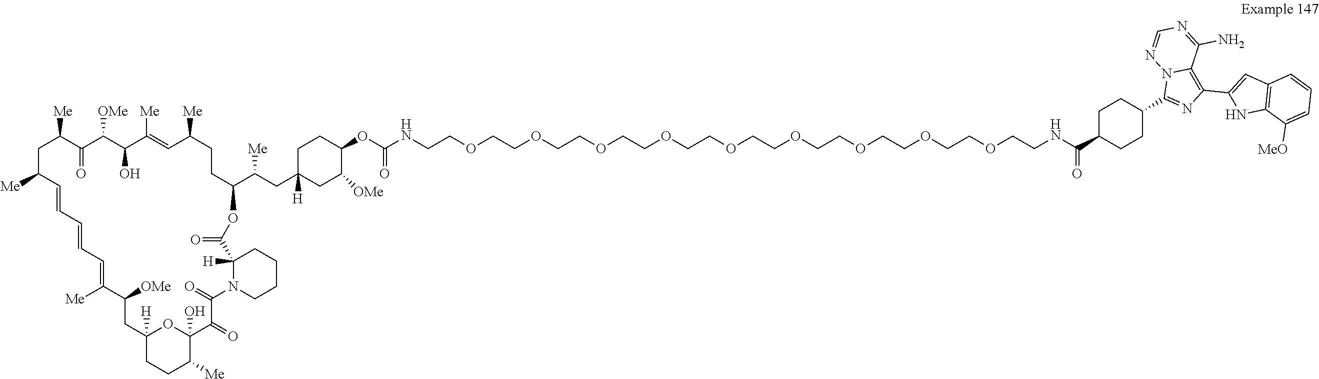 [SMILES: COc1cccc2cc(-c3nc([C@H]4CC[C@H](C(=O)NCCOCCOCCOCCOCCOCCOCCOCCOCCOCCNC(=O)O[C@@H]5CC[C@@H](C[C@@H](C)[C@@H]6CC[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12]

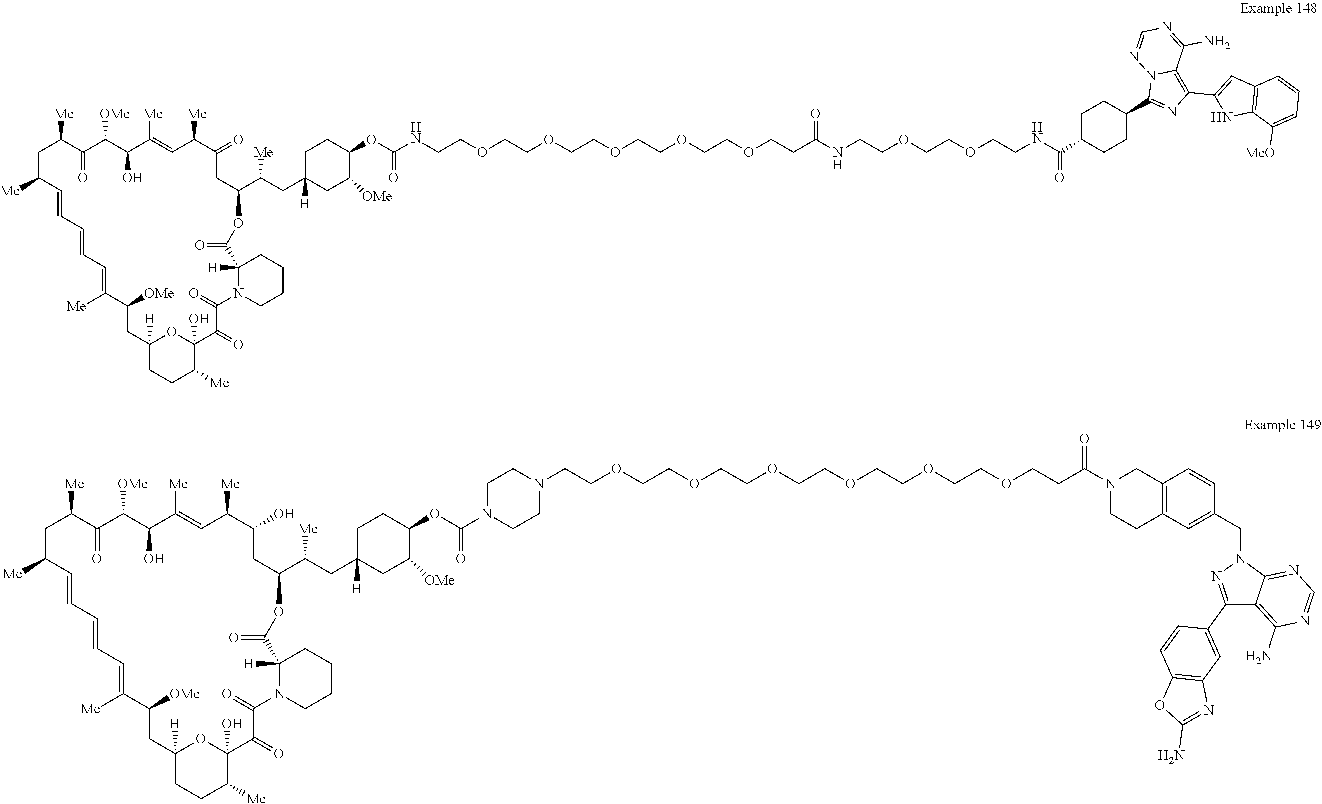

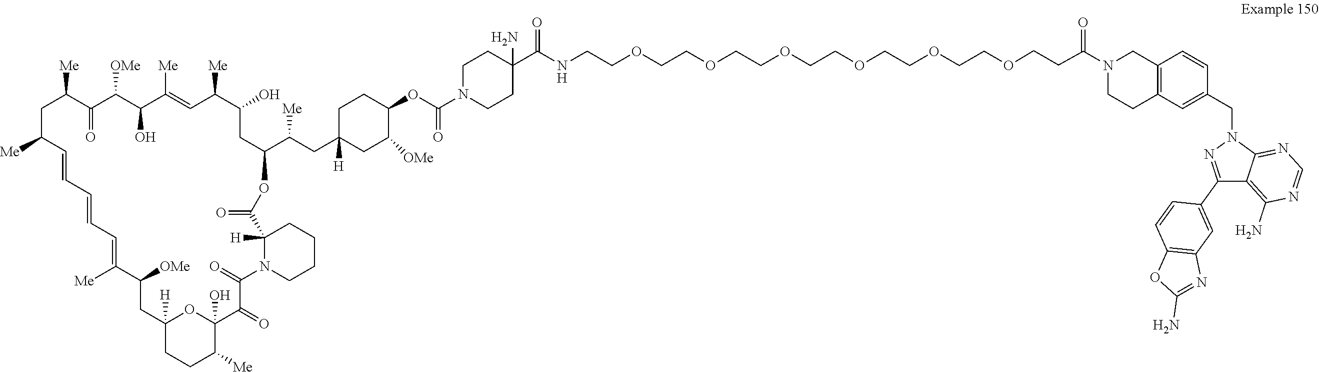 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCC(N)(C(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

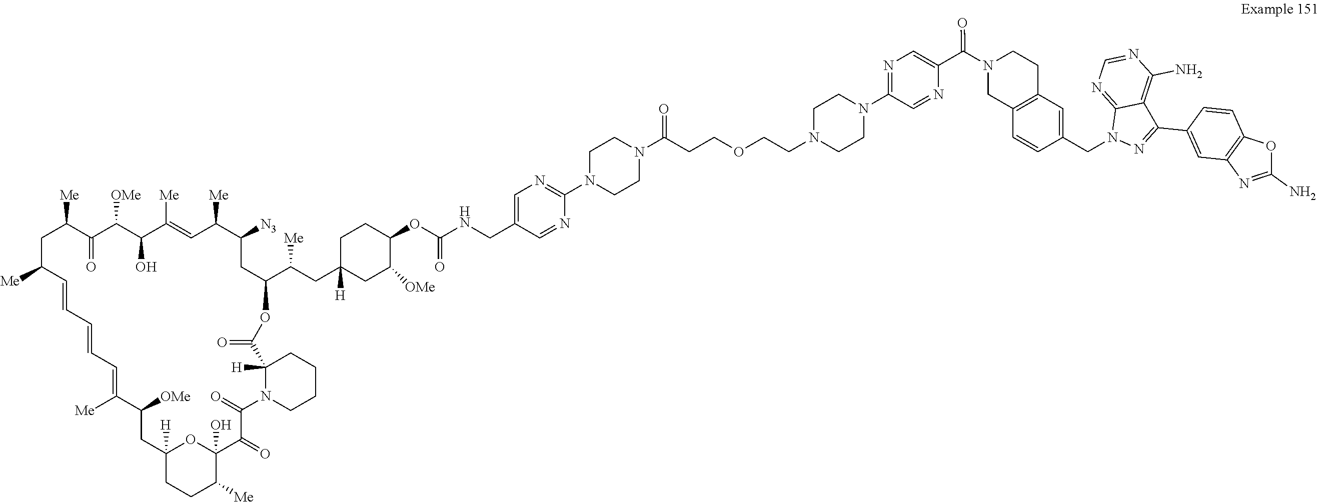 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6cnc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C]

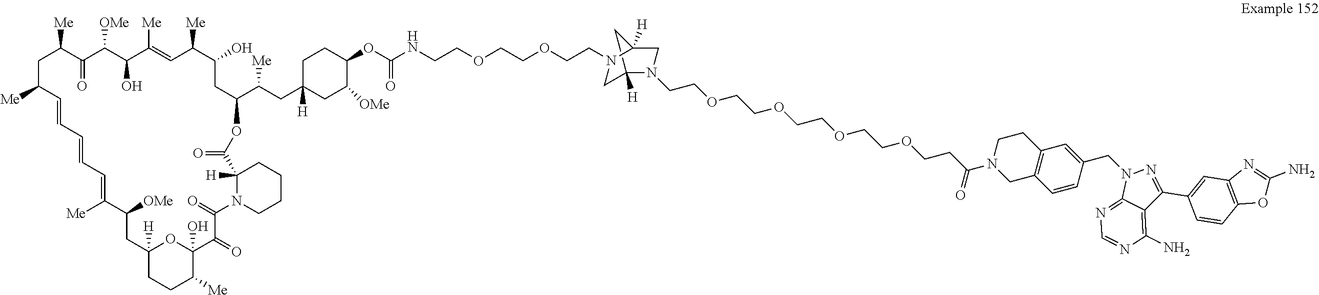 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCN3C[C@@H]4C[C@H]3CN4CCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a pharmaceutically acceptable salt or tautomer thereof.

Embodiment II-75. A pharmaceutical composition comprising a compound of any one of Embodiments II-1 to II-74, or a pharmaceutically acceptable salt thereof, and at least one of a pharmaceutically acceptable carrier, diluent, or excipient.

Embodiment II-76. A method of treating a disease or disorder mediated by mTOR comprising administering to the subject suffering from or susceptible to developing a disease or disorder mediated by mTOR a therapeutically effective amount of one or more compounds of any one of Embodiments II-1 to II-74, or a pharmaceutically acceptable salt thereof.

Embodiment II-77. A method of preventing a disease or disorder mediated by mTOR comprising administering to the subject suffering from or susceptible to developing a disease or disorder mediated by mTOR a therapeutically effective amount of one or more compounds of any one of Embodiments II-1 to II-74, or a pharmaceutically acceptable salt thereof.

Embodiment II-78. A method of reducing the risk of a disease or disorder mediated by mTOR comprising administering to the subject suffering from or susceptible to developing a disease or disorder mediated by mTOR a therapeutically effective amount of one or more compounds of any one of Embodiments II-1 to II-74, or a pharmaceutically acceptable salt thereof.

Embodiment II-79. The method of any one of Embodiments II-76 to II-78, wherein the disease is cancer or an immune-mediated disease.

Embodiment II-80. The method of Embodiment II-79, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment II-81. The method of Embodiment II-79, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment II-82. A method of treating cancer comprising administering to the subject a therapeutically effective amount of one or more compounds of any one of Embodiments II-1 to II-74, or a pharmaceutically acceptable salt thereof.

Embodiment II-83. The method of Embodiment II-82, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment II-84. A method of treating an immune-mediated disease comprising administering to the subject a therapeutically effective amount of one or more compounds of any one of Embodiments II-1 to II-74, or a pharmaceutically acceptable salt thereof.

Embodiment II-85. The method of Embodiment II-84, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment II-86. A method of treating an age related condition comprising administering to the subject a therapeutically effective amount of one or more compounds of any one of Embodiments II-1 to II-74, or a pharmaceutically acceptable salt thereof.

Embodiment II-87. The method of Embodiment II-86, wherein the age related condition is selected from sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes.

Embodiment II-88. A compound of any one of Embodiments II-1 to II-74, or a pharmaceutically acceptable salt thereof, for use in treating, preventing, or reducing the risk of a disease or condition mediated by mTOR.

Embodiment II-89. Use of a compound of any of Embodiments II-1 to II-74, or a pharmaceutically acceptable salt thereof, in the manufacture of a medicament for treating, preventing, or reducing the risk of a disease or disorder mediated by mTOR.

Embodiment II-90. A compound of any one of claims 1-74, or a pharmaceutically acceptable salt thereof, for use in treating cancer.

Embodiment II-91. Use of a compound of any one of Embodiments II-1 to II-74, or a pharmaceutically acceptable salt thereof, in the manufacture of a medicament for treating cancer.

Embodiment II-92. A compound of any one of Embodiments II-1 to II-74, or a pharmaceutically acceptable salt thereof, for use in treating an immune-mediated disease.

Embodiment II-93. Use of a compound of any one of Embodiments II-1 to II-74, or a pharmaceutically acceptable salt thereof, in the manufacture of a medicament for treating an immune-mediated disease.

Embodiment II-94. A compound of any one of Embodiments II-1 to II-74, or a pharmaceutically acceptable salt thereof, for use in treating an age related condition.

Embodiment II-95. Use of a compound of any one of Embodiments II-1 to II-74, or a pharmaceutically acceptable salt thereof, in the manufacture of a medicament for treating an age related condition.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Definitions used in the following examples and elsewhere herein are:
CH₂Cl₂, DCM Methylene chloride, Dichloromethane
CH₃CN, MeCN Acetonitrile
DIPEA Diisopropylethyl amine or Hunig's base
DMA Dimethylacetamide
DME Dimethoxyethane
DMF N,N-Dimethylformamide
EDCI 1-Ethyl-3-(3-dimethylaminopropyl)carbodiimide
EtOAc Ethyl acetate
h hour
H₂O Water
HCl Hydrochloric acid
HOBt Hydroxybenzotriazole
HPLC High-performance liquid chromatography
LCMS Liquid chromatography-mass spectrometry
MeOH Methanol
MTBE Methyl tert-butyl ether
Na₂SO₄ Sodium sulfate
PEG Polyethylene glycol
TBDMS tert-butyldimethylsilyl
TFA Trifluoroacetic acid
THF Tetrahydrofuran
TMS Tetramethylsilane

### Series 1 Bifunctional Rapalogs

A general structure of Series 1 bifunctional rapalogs is shown in Scheme 1 below. For these types of bifunctional rapalogs, the linker may include variations where q=0 to 30, such as q=1 to 7, and r=1 to 6. The linker amine can include substitutions, such as R═H and C1-C6 alkyl groups. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

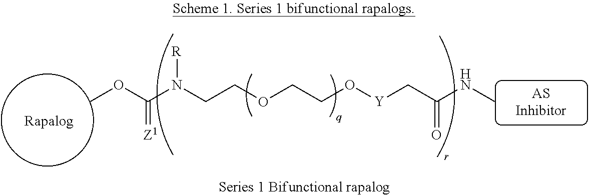

### Series 2 Bifunctional Rapalogs

A general structure of Series 2 bifunctional rapalogs is shown in Scheme 2 below. For these types of bifunctional rapalogs, the linker may include variations where q=0 to 30, such as q=1 to 7. The linker amine can include substitutions, such as R═H and C1-C6 alkyl groups. The pre-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

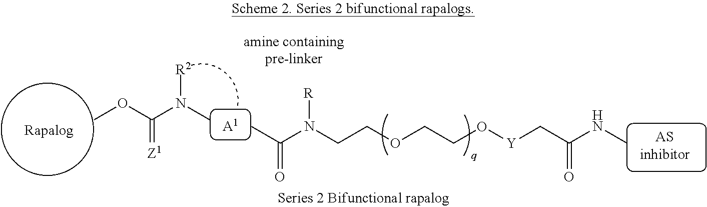

### Series 3 Bifunctional Rapalogs

A general structure of Series 3 bifunctional rapalogs is shown in Scheme 3 below. For these types of bifunctional rapalogs, the linker may include variations where q=0 to 30, such as q=1 to 7. The linker amine can include substitutions, such as R═H and C1-C6 alkyl groups. The post-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

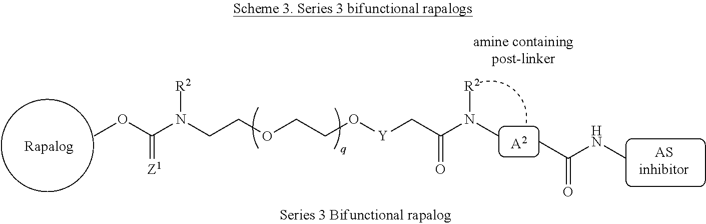

### Series 4 Bifunctional Rapalogs

A general structure of Series 4 bifunctional rapalogs is shown in Scheme 4 below. For these types of bifunctional rapalogs, the linker may include variations where q=0 to 30, such as q=1 to 7. The linker amine can include substitutions, such as R═H and C1-C6 alkyl groups. The pre- and post-linker amines can each include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

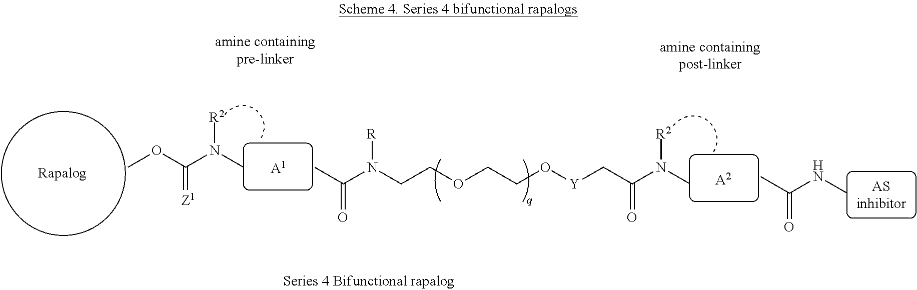

### Series 5 Bifunctional Rapalogs

A general structure of Series 5 bifunctional rapalogs is shown in Scheme 5 below. For these types of bifunctional rapalogs, the pre-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

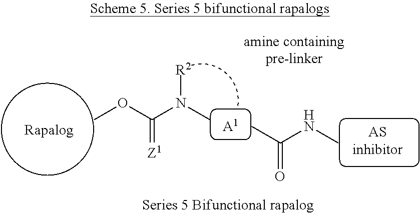

### Series 6 Bifunctional Rapalogs

A general structure of Series 6 bifunctional rapalogs is shown in Scheme 6 below. For these types of bifunctional rapalogs, the linker may include variations where q=0 to 30, such as q=1 to 7. The linker amines can include substitutions, such as R═H and C1-C6 alkyl groups. The post-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I and II), including variations found in Table 1 in the Examples Section. An mTOR active site inhibitor can attach to the linker via a primary or secondary amine, and may include variations found in Table 2 in the Examples Section.

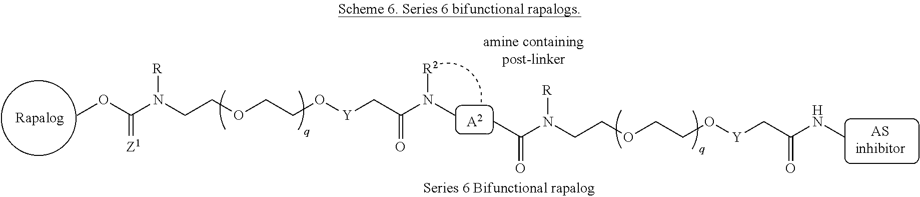

### Series 7 Bifunctional Rapalogs

A general structure of Series 7 bifunctional rapalogs is shown in Scheme 7 below. For these types of bifunctional rapalogs, the linker may include variations where q=0 to 30, such as q=1 to 7. The linker amine can include substitutions, such as R═H and C1-C6 alkyl groups. The pre- and post-linker amines can each include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I or II), including variations found in Table 1 in the Examples Section. An mTOR active site inhibitor can attach to the linker via a primary or secondary amine, and may include variations found in Table 2 in the Examples Section.

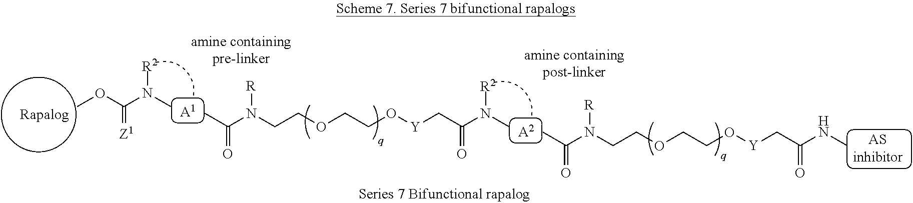

### Series 8 Bifunctional Rapalogs

A general structure of Series 8 bifunctional rapalogs is shown in Scheme 8 below. For these types of bifunctional rapalogs, the linker may include variations where q=0 to 30, such as q=1 to 7. The linker amine can include substitutions, such as R═H and C1-C6 alkyl groups. The post-linker amine can include substitutions, such as R²═H, C1-C6 alkyl groups, and cycloalkyl including 4 to 8-membered rings. The carbamate moiety, where Z¹═O or S, can be attached to the rapalog at R⁴⁰ or R²⁸ (Formula I or II), including variations found in Table 1 in the Examples Section. An mTOR active site inhibitor can attach to the linker via a primary or secondary amine, and may include variations found in Table 2 in the Examples Section.

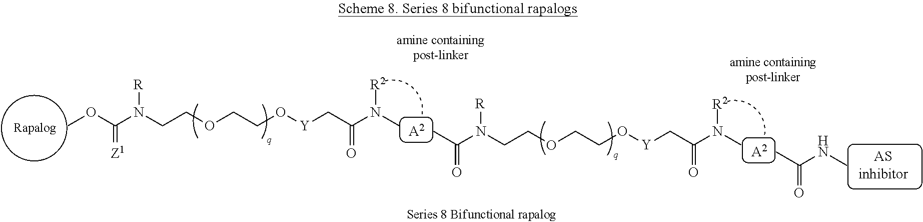

TABLE 1
Carbonate and thiocarbonate containing rapalog monomers.

| Carbonate containing rapalog | |
|---|---|
| 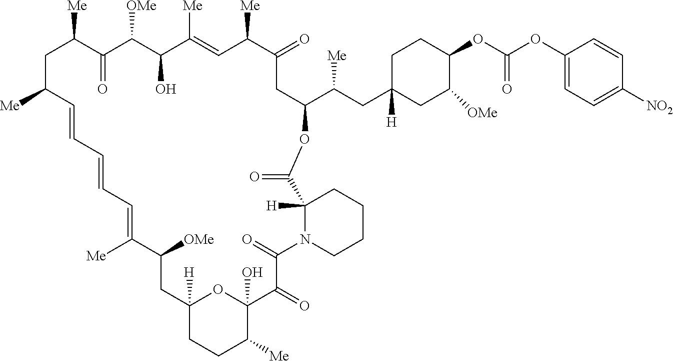 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)CC(=O)[C@H](C)/C=C(\C)[C@@H](O)[C@@H](OC)C(=O)[C@H](C)C[C@H](C)/C=C/C=C/C=C/1C] | Monomer 1 |
| 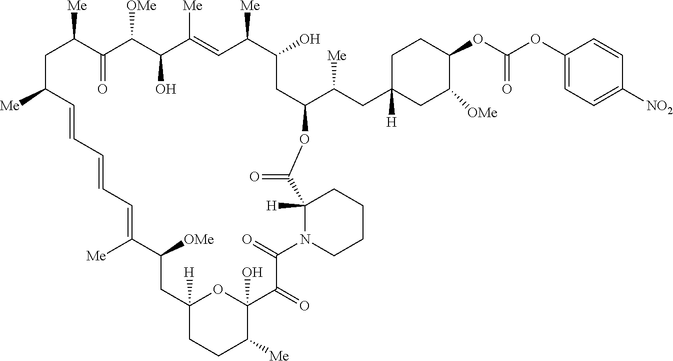 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | Monomer 2 |
| 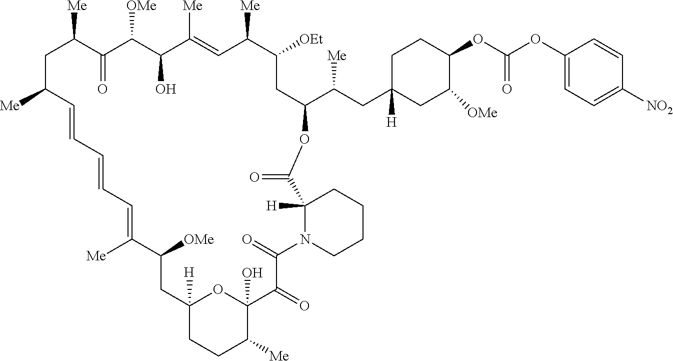 [SMILES: CCO[C@@H]1C[C@@H]([C@H](C)C[C@@H]2CC[C@@H](OC(=O)Oc3ccc([N+](=O)[O-])cc3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | Monomer 3 |
| 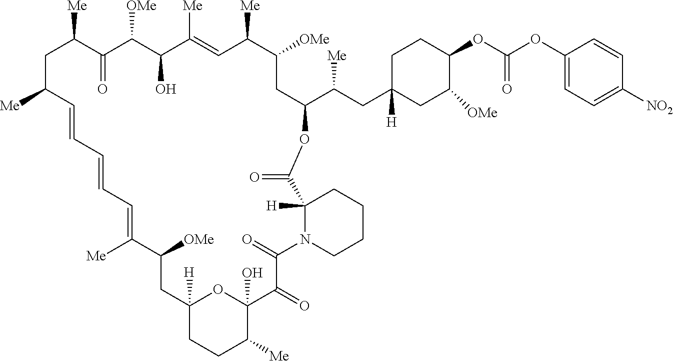 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | Monomer 4 |
| 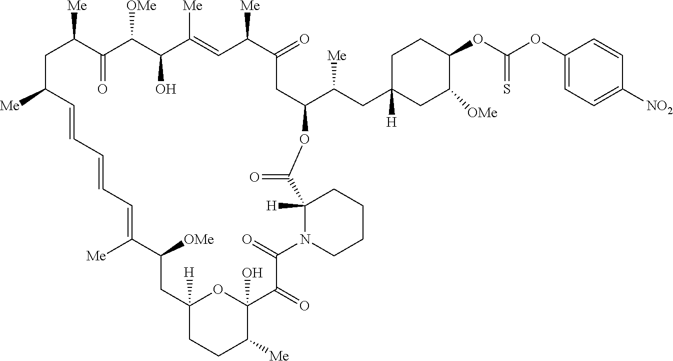 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)Oc3ccc([N+](=O)[O-])cc3)[C@H](OC)C2)CC(=O)[C@H](C)/C=C(\C)[C@@H](O)[C@@H](OC)C(=O)[C@H](C)C[C@H](C)/C=C/C=C/C=C/1C] | Monomer 5 |
| 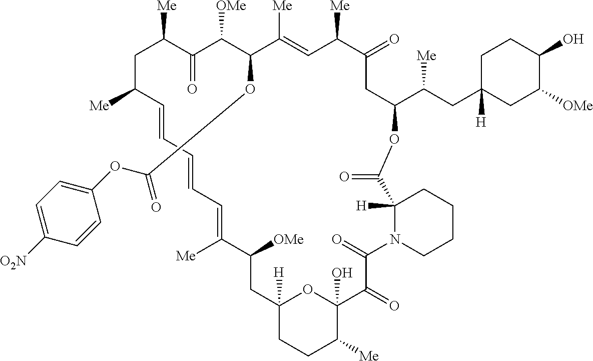 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](OC(=O)Oc2ccc([N+](=O)[O-])cc2)[C@@H](OC)C(=O)[C@H](C)C[C@H](C)/C=C/C=C/C=C/1C] | Monomer 6 |
| 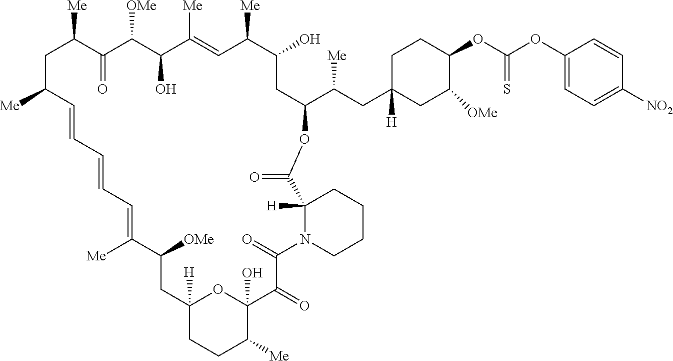 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | Monomer 7 |
| 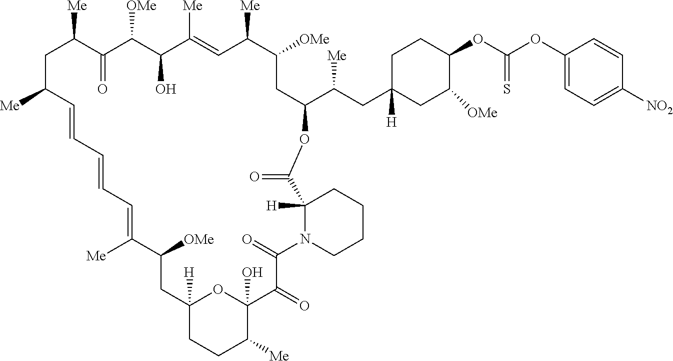 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)Oc3ccc([N+](=O)[O-])cc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | Monomer 8 |
| 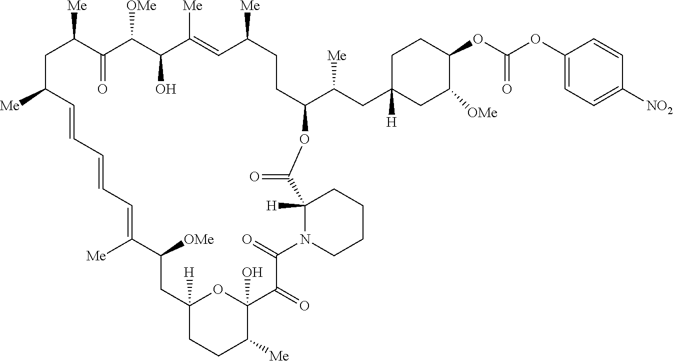 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)CC[C@H](C)/C=C(\C)[C@@H](O)[C@@H](OC)C(=O)[C@H](C)C[C@H](C)/C=C/C=C/C=C/1C] | Monomer 9 |
| 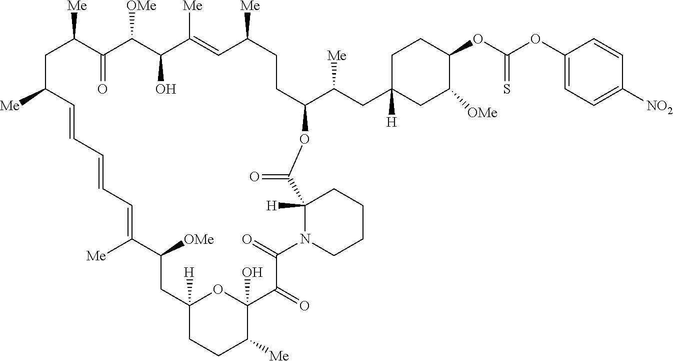 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)Oc3ccc([N+](=O)[O-])cc3)[C@H](OC)C2)CC[C@H](C)/C=C(\C)[C@@H](O)[C@@H](OC)C(=O)[C@H](C)C[C@H](C)/C=C/C=C/C=C/1C] | Monomer 10 |
| 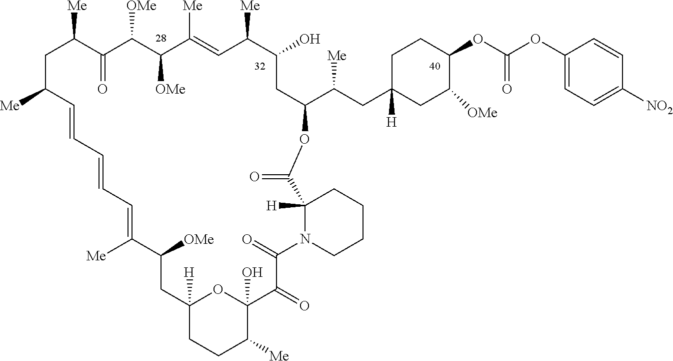 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](OC)[C@@H](OC)C(=O)[C@H](C)C[C@H](C)/C=C/C=C/C=C/1C] | Monomer 11 |
| 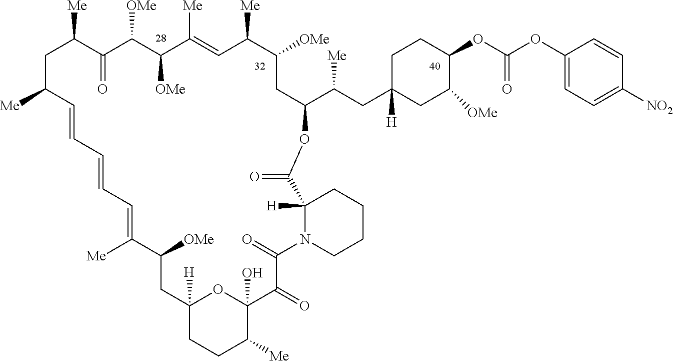 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](OC)[C@H](C)/C=C(\C)[C@@H](OC)[C@@H](OC)C(=O)[C@H](C)C[C@H](C)/C=C/C=C/C=C/1C] | Monomer 12 |
| 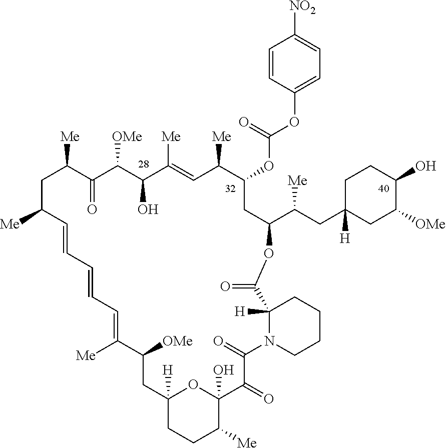 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC(=O)Oc2ccc([N+](=O)[O-])cc2)[C@H](C)/C=C(\C)[C@@H](O)[C@@H](OC)C(=O)[C@H](C)C[C@H](C)/C=C/C=C/C=C/1C] | Monomer 13 |
| 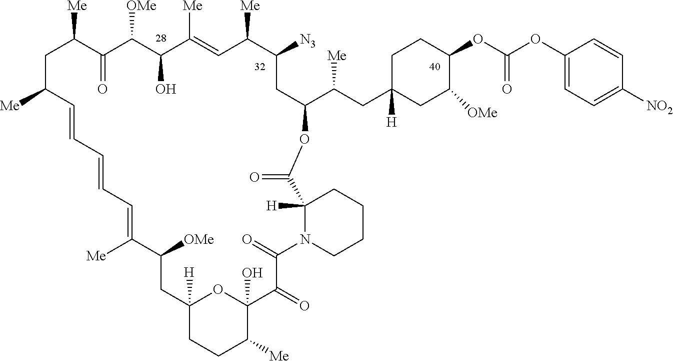 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] | Monomer 14 |
| 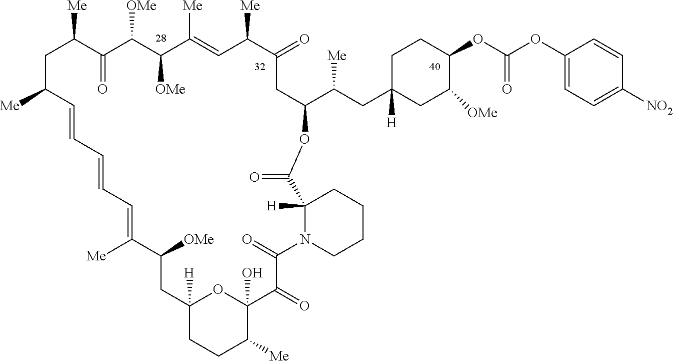 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)CC(=O)[C@H](C)/C=C(\C)[C@@H](OC)[C@@H](OC)C(=O)[C@H](C)C[C@H](C)/C=C/C=C/C=C/1C] | Monomer 15 |
| 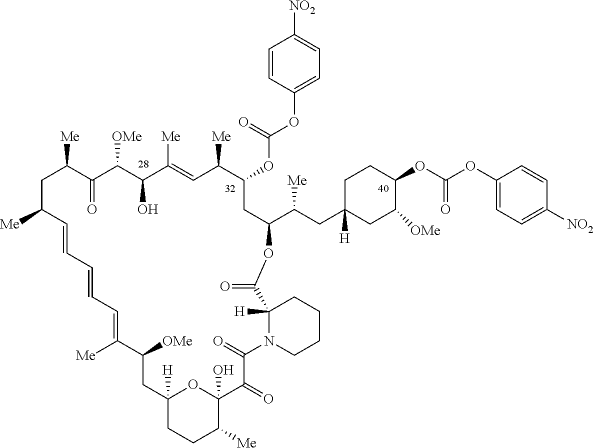 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](OC(=O)Oc2ccc([N+](=O)[O-])cc2)[C@H](C)/C=C(\C)[C@@H](O)[C@@H](OC)C(=O)[C@H](C)C[C@H](C)/C=C/C=C/C=C/1C] | Monomer 16 |
| 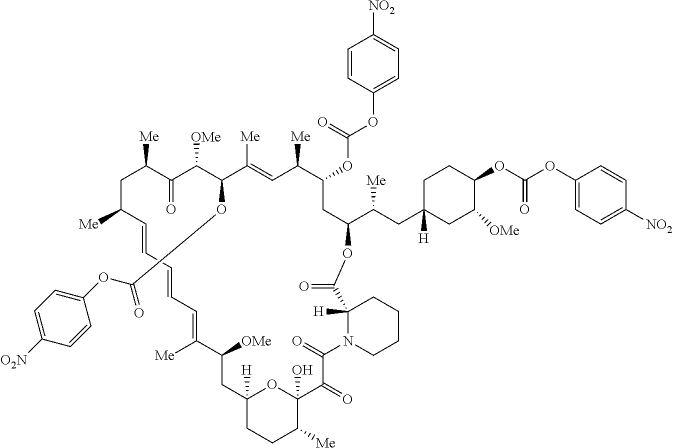 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](OC(=O)Oc2ccc([N+](=O)[O-])cc2)[C@H](C)/C=C(\C)[C@@H](OC(=O)Oc2ccc([N+](=O)[O-])cc2)[C@@H](OC)C(=O)[C@H](C)C[C@H](C)/C=C/C=C/C=C/1C] | Monomer 17 |

TABLE 2
Active Site inhibitor monomers.
Active Site inhibitor monomers

| | |
|---|---|
| 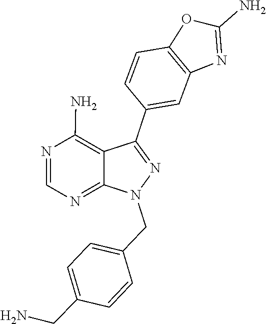 [SMILES: NCc1ccc(Cn2nc(-c3ccc4oc(N)nc4c3)c3c(N)ncnc32)cc1] | Monomer A |
| 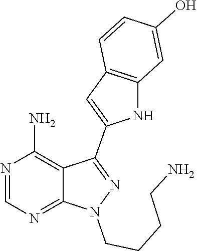 [SMILES: NCCCCn1nc(-c2cc3ccc(O)cc3[nH]2)c2c(N)ncnc21] | Monomer B |
| 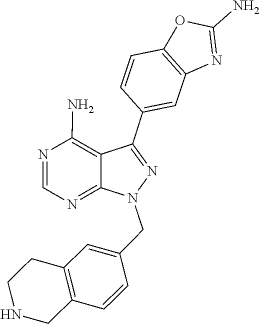 [SMILES: Nc1nc2cc(-c3nn(Cc4ccc5c(c4)CCNC5)c4ncnc(N)c34)ccc2o1] | Monomer C |
| 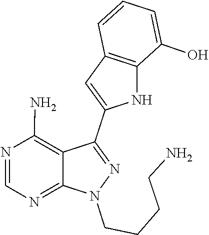 [SMILES: NCCCCn1nc(-c2cc3cccc(O)c3[nH]2)c2c(N)ncnc21] | Monomer D |
| 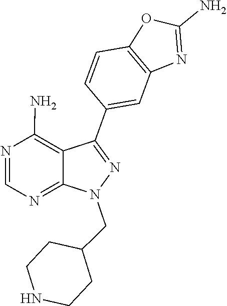 [SMILES: Nc1nc2cc(-c3nn(CC4CCNCC4)c4ncnc(N)c34)ccc2o1] | Monomer E |
| 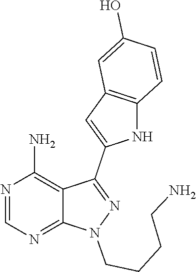 [SMILES: NCCCCn1nc(-c2cc3cc(O)ccc3[nH]2)c2c(N)ncnc21] | Monomer F |
| 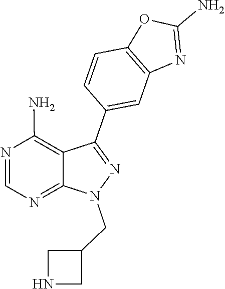 [SMILES: Nc1nc2cc(-c3nn(CC4CNC4)c4ncnc(N)c34)ccc2o1] | Monomer G |
| 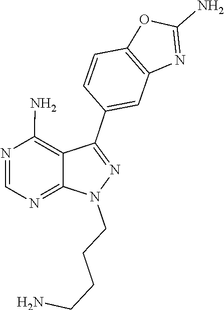 [SMILES: NCCCCn1nc(-c2ccc3oc(N)nc3c2)c2c(N)ncnc21] | Monomer H |
| 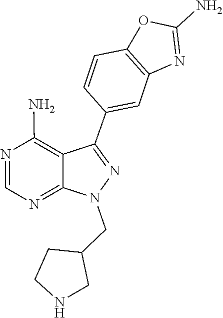 [SMILES: Nc1nc2cc(-c3nn(CC4CCNC4)c4ncnc(N)c34)ccc2o1] | Monomer I |
| 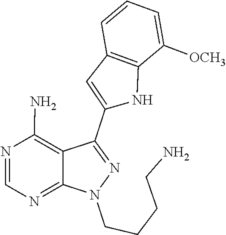 [SMILES: COc1cccc2cc(-c3nn(CCCCN)c4ncnc(N)c34)[nH]c12] | Monomer J |
| 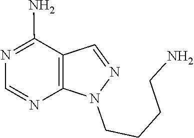 [SMILES: NCCCCn1ncc2c(N)ncnc21] | Monomer K |
| 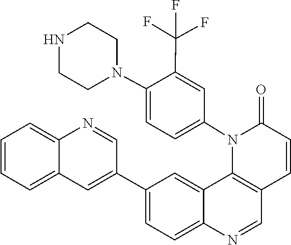 [SMILES: O=c1ccc2cnc3ccc(-c4cnc5ccccc5c4)cc3c2n1-c1ccc(N2CCNCC2)c(C(F)(F)F)c1] | Monomer L |
| 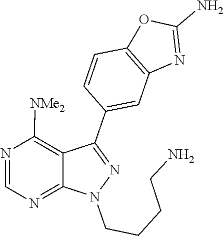 [SMILES: CN(C)c1ncnc2c1c(-c1ccc3oc(N)nc3c1)nn2CCCCN] | Monomer M |
| 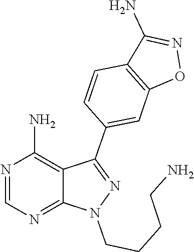 [SMILES: NCCCCn1nc(-c2ccc3c(N)noc3c2)c2c(N)ncnc21] | Monomer N |
| 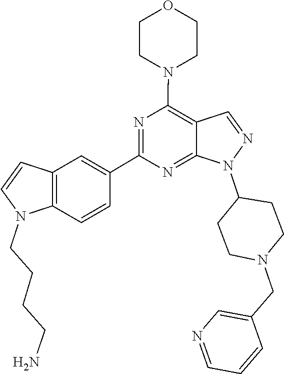 [SMILES: NCCCCn1ccc2cc(-c3nc(N4CCOCC4)c4cnn(C5CCN(Cc6cccnc6)CC5)c4n3)ccc21] | Monomer O |
| 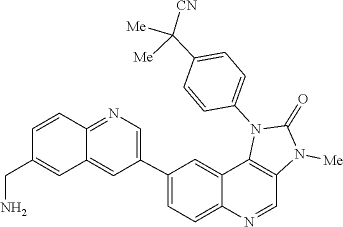 [SMILES: Cn1c(=O)n(-c2ccc(C(C)(C)C#N)cc2)c2c3cc(-c4cnc5ccc(CN)cc5c4)ccc3ncc21] | Monomer P |
| 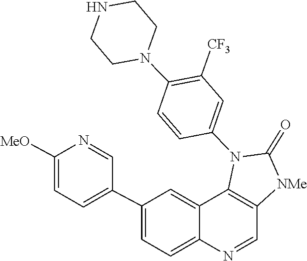 [SMILES: COc1ccc(-c2ccc3ncc4c(c3c2)n(-c2ccc(N3CCNCC3)c(C(F)(F)F)c2)c(=O)n4C)cn1] | Monomer Q |
| 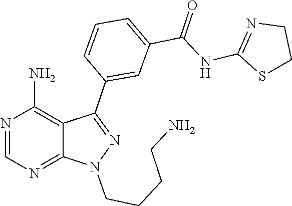 [SMILES: NCCCCn1nc(-c2cccc(C(=O)NC3=NCCS3)c2)c2c(N)ncnc21] | Monomer R |
| 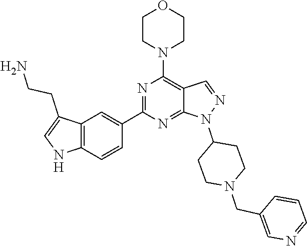 [SMILES: NCCc1c[nH]c2ccc(-c3nc(N4CCOCC4)c4cnn(C5CCN(Cc6cccnc6)CC5)c4n3)cc12] | Monomer S |
| 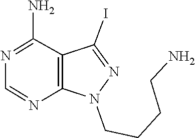 | Monomer T |
| 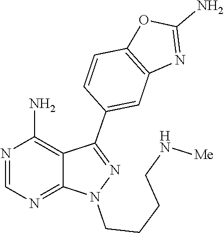 [SMILES: CNCCCCn1nc(-c2ccc3oc(N)nc3c2)c2c(N)ncnc21] | Monomer U |
| 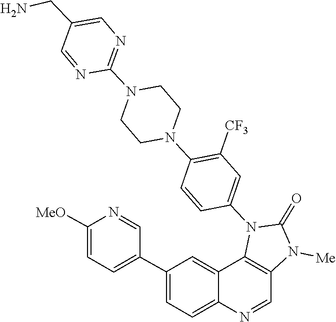 [SMILES: COc1ccc(-c2ccc3ncc4c(c3c2)n(-c2ccc(N3CCN(c5ncc(CN)cn5)CC3)c(C(F)(F)F)c2)c(=O)n4C)cn1] | Monomer V |
| 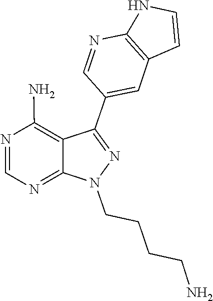 [SMILES: NCCCCn1nc(-c2cnc3[nH]ccc3c2)c2c(N)ncnc21] | Monomer W |
| 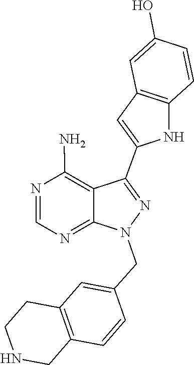 [SMILES: Nc1ncnc2c1c(-c1cc3cc(O)ccc3[nH]1)nn2Cc1ccc2c(c1)CCNC2] | Monomer X |
| 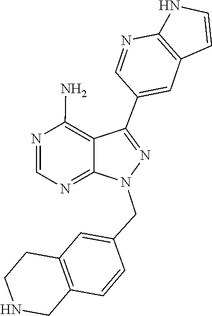 [SMILES: Nc1ncnc2c1c(-c1cnc3[nH]ccc3c1)nn2Cc1ccc2c(c1)CCNC2] | Monomer Y |
| 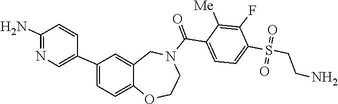 [SMILES: Cc1c(C(=O)N2CCOc3ccc(-c4ccc(N)nc4)cc3C2)ccc(S(=O)(=O)CCN)c1F] | Monomer Z |
| 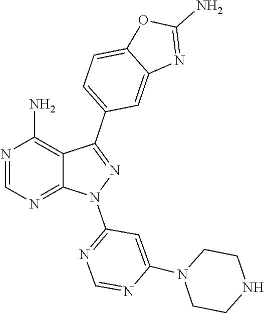 [SMILES: Nc1nc2cc(-c3nn(-c4cc(N5CCNCC5)ncn4)c4ncnc(N)c34)ccc2o1] | Monomer AA |
| 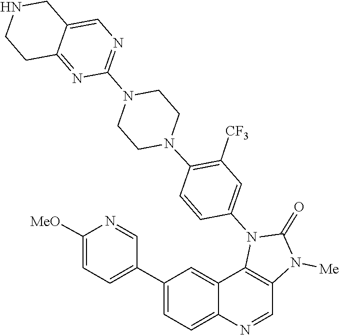 [SMILES: COc1ccc(-c2ccc3ncc4c(c3c2)n(-c2ccc(N3CCN(c5ncc6c(n5)CCNC6)CC3)c(C(F)(F)F)c2)c(=O)n4C)cn1] | Monomer AB |
| 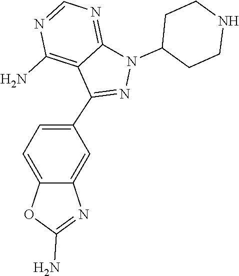 [SMILES: Nc1nc2cc(-c3nn(C4CCNCC4)c4ncnc(N)c34)ccc2o1] | Monomer AC |
| 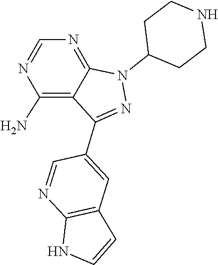 [SMILES: Nc1ncnc2c1c(-c1cnc3[nH]ccc3c1)nn2C1CCNCC1] | Monomer AD |
| 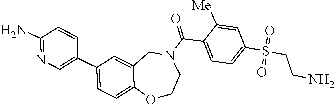 [SMILES: Cc1cc(S(=O)(=O)CCN)ccc1C(=O)N1CCOc2ccc(-c3ccc(N)nc3)cc2C1] | Monomer AE |
| 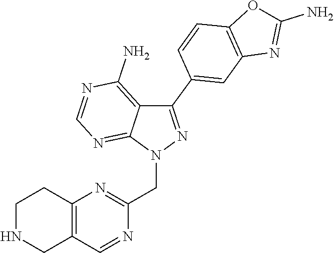 [SMILES: Nc1nc2cc(-c3nn(Cc4ncc5c(n4)CCNC5)c4ncnc(N)c34)ccc2o1] | Monomer AF |
| 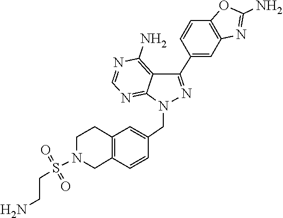 [SMILES: NCCS(=O)(=O)N1CCc2cc(Cn3nc(-c4ccc5oc(N)nc5c4)c4c(N)ncnc43)ccc2C1] | Monomer AG |
| 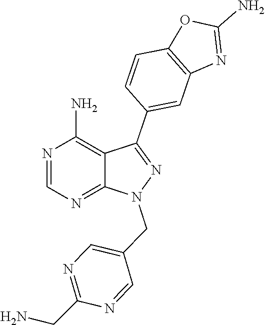 [SMILES: NCc1ncc(Cn2nc(-c3ccc4oc(N)nc4c3)c3c(N)ncnc32)cn1] | Monomer AH |
| 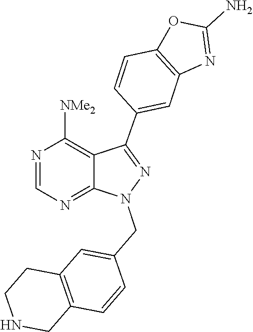 [SMILES: CN(C)c1ncnc2c1c(-c1ccc3oc(N)nc3c1)nn2Cc1ccc2c(c1)CCNC2] | Monomer AI |

TABLE 3
Active Site inhibitor monomers
Active Site Inhibitor

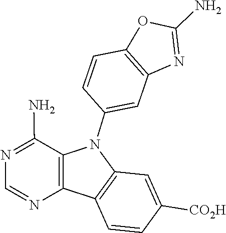 [SMILES: Nc1nc2cc(-n3c4cc(C(=O)O)ccc4c4ncnc(N)c43)ccc2o1]

Monomer AJ

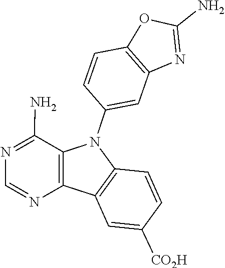 [SMILES: Nc1nc2cc(-n3c4ccc(C(=O)O)cc4c4ncnc(N)c43)ccc2o1]

Monomer AK

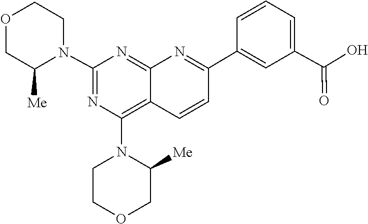 [SMILES: C[C@H]1COCCN1c1nc(N2CCOC[C@@H]2C)c2ccc(-c3cccc(C(=O)O)c3)nc2n1]

Monomer AL

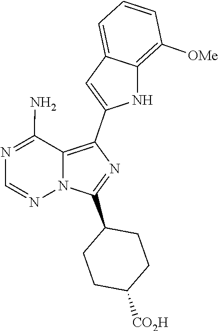 [SMILES: COc1cccc2cc(-c3nc([C@H]4CC[C@H](C(=O)O)CC4)n4ncnc(N)c34)[nH]c12]

Monomer AM

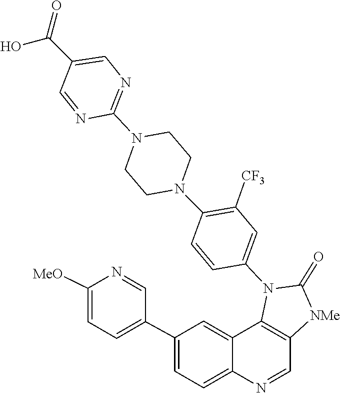 [SMILES: COc1ccc(-c2ccc3ncc4c(c3c2)n(-c2ccc(N3CCN(c5ncc(C(=O)O)cn5)CC3)c(C(F)(F)F)c2)c(=O)n4C)cn1]

Monomer AN

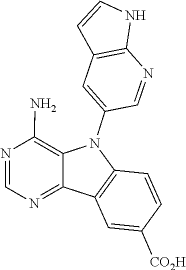 [SMILES: Nc1ncnc2c3cc(C(=O)O)ccc3n(-c3cnc4[nH]ccc4c3)c12]

Monomer AO

TABLE 4
Amine containing pre- and post-linkers.
Amide containing block

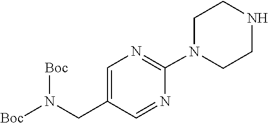 [SMILES: CC(C)(C)OC(=O)N(Cc1cnc(N2CCNCC2)nc1)C(=O)OC(C)(C)C]

Building block A

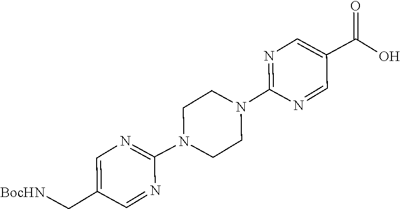 [SMILES: CC(C)(C)OC(=O)NCc1cnc(N2CCN(c3ncc(C(=O)O)cn3)CC2)nc1]

Building block B

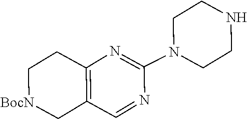 [SMILES: CC(C)(C)OC(=O)N1CCc2nc(N3CCNCC3)ncc2C1]

Building block C

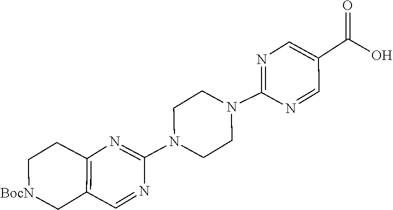 [SMILES: CC(C)(C)OC(=O)N1CCc2nc(N3CCN(c4ncc(C(=O)O)cn4)CC3)ncc2C1]

Building block D

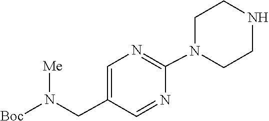 [SMILES: CN(Cc1cnc(N2CCNCC2)nc1)C(=O)OC(C)(C)C]

Building block E

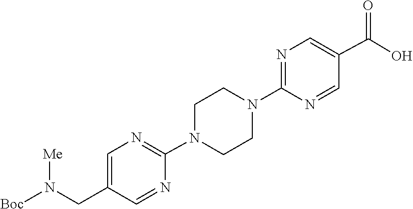 [SMILES: CN(Cc1cnc(N2CCN(c3ncc(C(=O)O)cn3)CC2)nc1)C(=O)OC(C)(C)C]

Building block F

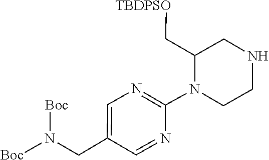

Building block G

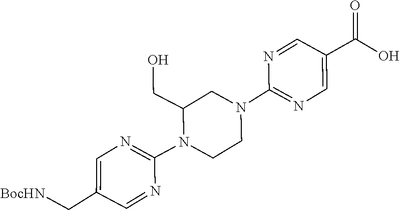 [SMILES: CC(C)(C)OC(=O)NCc1cnc(N2CCN(c3ncc(C(=O)O)cn3)CC2CO)nc1]

Building block H

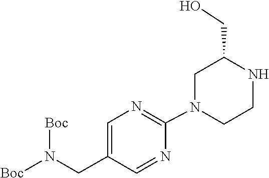 [SMILES: CC(C)(C)OC(=O)N(Cc1cnc(N2CCN[C@@H](CO)C2)nc1)C(=O)OC(C)(C)C]

Building block I

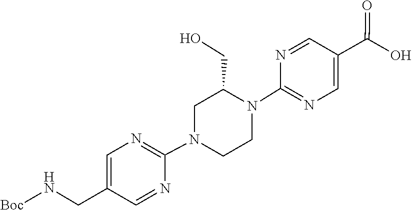 [SMILES: CC(C)(C)OC(=O)NCc1cnc(N2CCN(c3ncc(C(=O)O)cn3)[C@@H](CO)C2)nc1]

Building block J

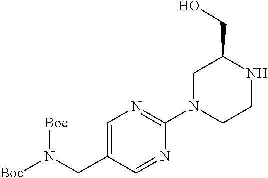 [SMILES: CC(C)(C)OC(=O)N(Cc1cnc(N2CCN[C@H](CO)C2)nc1)C(=O)OC(C)(C)C]

Building block K

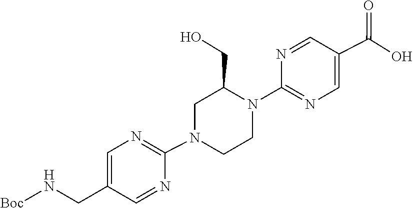 [SMILES: CC(C)(C)OC(=O)NCc1cnc(N2CCN(c3ncc(C(=O)O)cn3)[C@H](CO)C2)nc1]

Building block L

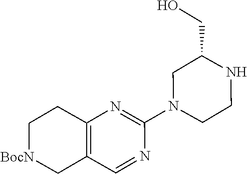 [SMILES: CC(C)(C)OC(=O)N1CCc2nc(N3CCN[C@@H](CO)C3)ncc2C1]

Building block M

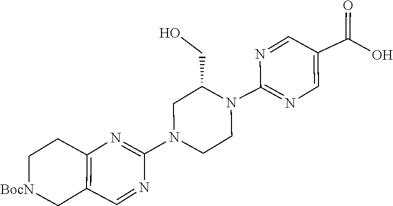 [SMILES: CC(C)(C)OC(=O)N1CCc2nc(N3CCN(c4ncc(C(=O)O)cn4)[C@@H](CO)C3)ncc2C1]

Building block N

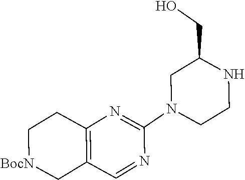 [SMILES: CC(C)(C)OC(=O)N1CCc2nc(N3CCN[C@H](CO)C3)ncc2C1]

Building block O

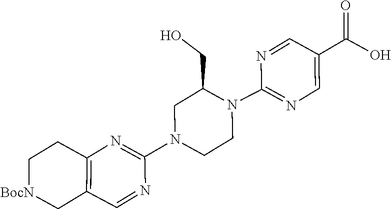 [SMILES: CC(C)(C)OC(=O)N1CCc2nc(N3CCN(c4ncc(C(=O)O)cn4)[C@H](CO)C3)ncc2C1]

Building block P

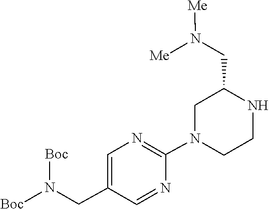 [SMILES: CN(C)C[C@H]1CN(c2ncc(CN(C(=O)OC(C)(C)C)C(=O)OC(C)(C)C)cn2)CCN1]

Building block Q

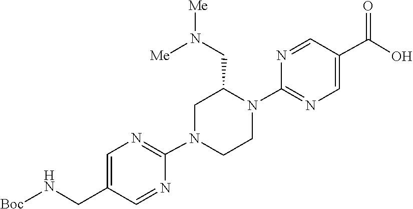 [SMILES: CN(C)C[C@H]1CN(c2ncc(CNC(=O)OC(C)(C)C)cn2)CCN1c1ncc(C(=O)O)cn1]

Building block R

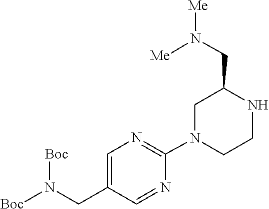 [SMILES: CN(C)C[C@@H]1CN(c2ncc(CN(C(=O)OC(C)(C)C)C(=O)OC(C)(C)C)cn2)CCN1]

Building block S

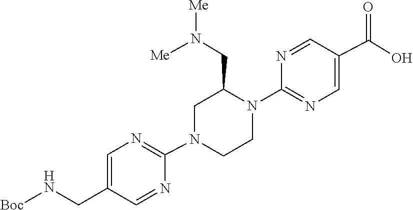 [SMILES: CN(C)C[C@@H]1CN(c2ncc(CNC(=O)OC(C)(C)C)cn2)CCN1c1ncc(C(=O)O)cn1]

Building block T

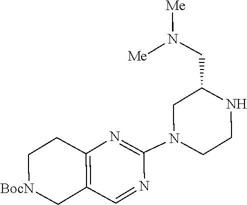 [SMILES: CN(C)C[C@H]1CN(c2ncc3c(n2)CCN(C(=O)OC(C)(C)C)C3)CCN1]

Building block U

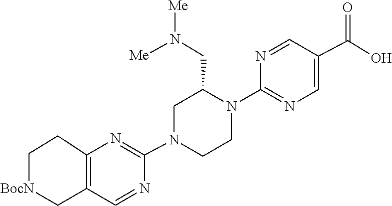 [SMILES: CN(C)C[C@H]1CN(c2ncc3c(n2)CCN(C(=O)OC(C)(C)C)C3)CCN1c1ncc(C(=O)O)cn1]

Building block V

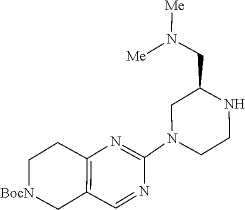 [SMILES: CN(C)C[C@@H]1CN(c2ncc3c(n2)CCN(C(=O)OC(C)(C)C)C3)CCN1]

Building block W

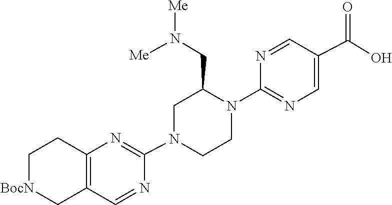 [SMILES: CN(C)C[C@@H]1CN(c2ncc3c(n2)CCN(C(=O)OC(C)(C)C)C3)CCN1c1ncc(C(=O)O)cn1]

Building block X

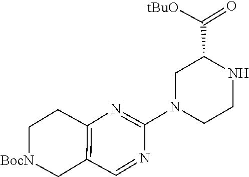 [SMILES: CC(C)(C)OC(=O)[C@H]1CN(c2ncc3c(n2)CCN(C(=O)OC(C)(C)C)C3)CCN1]

Building block Y

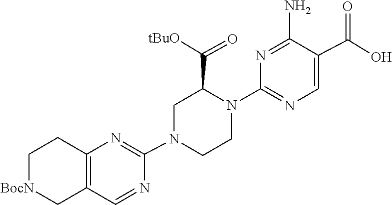 [SMILES: CC(C)(C)OC(=O)[C@@H]1CN(c2ncc3c(n2)CCN(C(=O)OC(C)(C)C)C3)CCN1c1ncc(C(=O)O)c(N)n1]

Building block Z

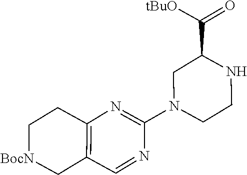 [SMILES: CC(C)(C)OC(=O)[C@@H]1CN(c2ncc3c(n2)CCN(C(=O)OC(C)(C)C)C3)CCN1]

Building block AA

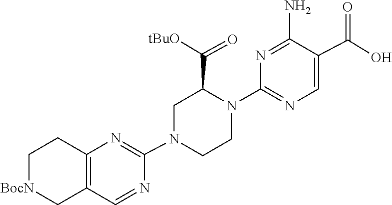 [SMILES: CC(C)(C)OC(=O)[C@@H]1CN(c2ncc3c(n2)CCN(C(=O)OC(C)(C)C)C3)CCN1c1ncc(C(=O)O)c(N)n1]

Building block AB

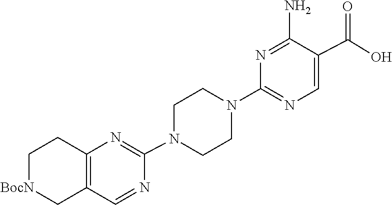 [SMILES: CC(C)(C)OC(=O)N1CCc2nc(N3CCN(c4ncc(C(=O)O)c(N)n4)CC3)ncc2C1]

Building block AC

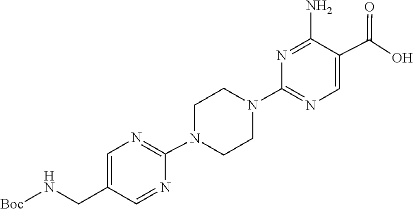 [SMILES: CC(C)(C)OC(=O)NCc1cnc(N2CCN(c3ncc(C(=O)O)c(N)n3)CC2)nc1]

Building block AD

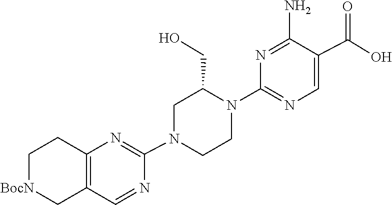 [SMILES: CC(C)(C)OC(=O)N1CCc2nc(N3CCN(c4ncc(C(=O)O)c(N)n4)[C@@H](CO)C3)ncc2C1]

Building block AE

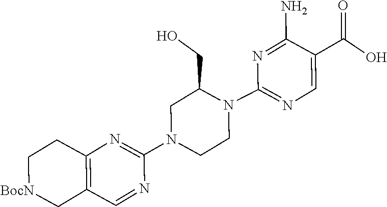 [SMILES: CC(C)(C)OC(=O)N1CCc2nc(N3CCN(c4ncc(C(=O)O)c(N)n4)[C@H](CO)C3)ncc2C1]

Building block AF

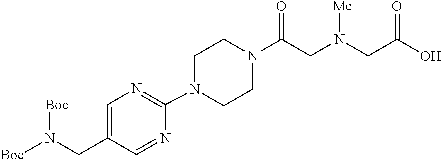 [SMILES: CN(CC(=O)O)CC(=O)N1CCN(c2ncc(CN(C(=O)OC(C)(C)C)C(=O)OC(C)(C)C)cn2)CC1]

Building block AG

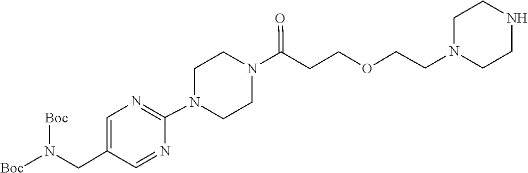 [SMILES: CC(C)(C)OC(=O)N(Cc1cnc(N2CCN(C(=O)CCOCCN3CCNCC3)CC2)nc1)C(=O)OC(C)(C)C]

Building block AH

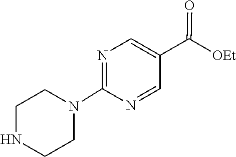 [SMILES: CCOC(=O)c1cnc(N2CCNCC2)nc1]

Building block AI

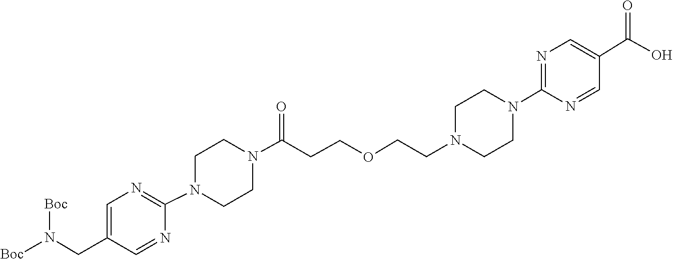 [SMILES: CC(C)(C)OC(=O)N(Cc1cnc(N2CCN(C(=O)CCOCCN3CCN(c4ncc(C(=O)O)cn4)CC3)CC2)nc1)C(=O)OC(C)(C)C]

Building block AJ

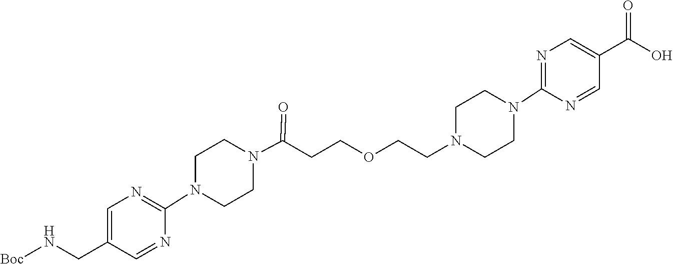 [SMILES: CC(C)(C)OC(=O)NCc1cnc(N2CCN(C(=O)CCOCCN3CCN(c4ncc(C(=O)O)cn4)CC3)CC2)nc1]

Building block AK

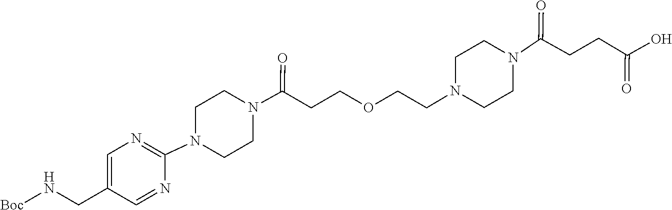 [SMILES: CC(C)(C)OC(=O)NCc1cnc(N2CCN(C(=O)CCOCCN3CCN(C(=O)CCC(=O)O)CC3)CC2)nc1]

Building block AL

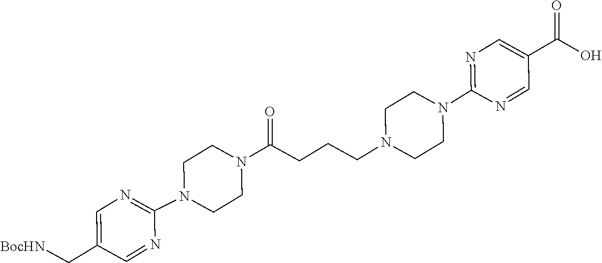 [SMILES: CC(C)(C)OC(=O)NCc1cnc(N2CCN(C(=O)CCCN3CCN(c4ncc(C(=O)O)cn4)CC3)CC2)nc1]

Building block AM

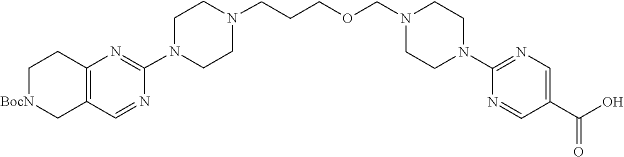 [SMILES: CC(C)(C)OC(=O)N1CCc2nc(N3CCN(CCCOCN4CCN(c5ncc(C(=O)O)cn5)CC4)CC3)ncc2C1]

Building block AN

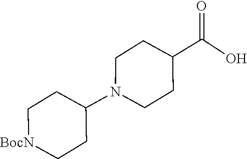 [SMILES: CC(C)(C)OC(=O)N1CCC(N2CCC(C(=O)O)CC2)CC1]

Building block AO

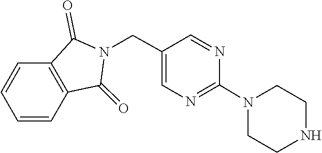 [SMILES: O=C1c2ccccc2C(=O)N1Cc1cnc(N2CCNCC2)nc1]

Building block AP

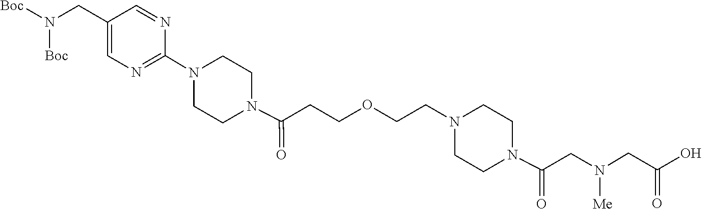 [SMILES: CN(CC(=O)O)CC(=O)N1CCN(CCOCCC(=O)N2CCN(c3ncc(CN(C(=O)OC(C)(C)C)C(=O)OC(C)(C)C)cn3)CC2)CC1]

Building block AQ

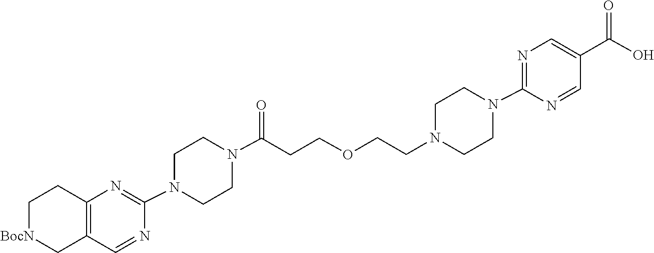 [SMILES: CC(C)(C)OC(=O)N1CCc2nc(N3CCN(C(=O)CCOCCN4CCN(c5ncc(C(=O)O)cn5)CC4)CC3)ncc2C1]

Building block AR

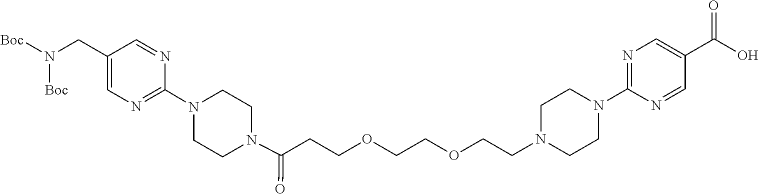 [SMILES: CC(C)(C)OC(=O)N(Cc1cnc(N2CCN(C(=O)CCOCCOCCN3CCN(c4ncc(C(=O)O)cn4)CC3)CC2)nc1)C(=O)OC(C)(C)C]

Building block AS

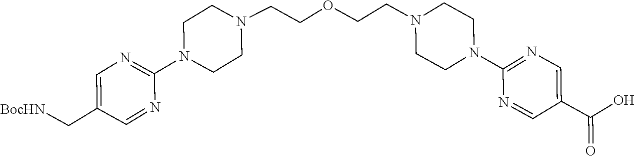 [SMILES: CC(C)(C)OC(=O)NCc1cnc(N2CCN(CCOCCN3CCN(c4ncc(C(=O)O)cn4)CC3)CC2)nc1]

Building block AT

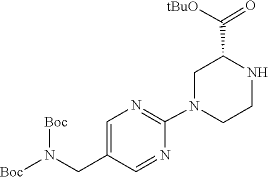 [SMILES: CC(C)(C)OC(=O)[C@H]1CN(c2ncc(CN(C(=O)OC(C)(C)C)C(=O)OC(C)(C)C)cn2)CCN1]

Building block AU

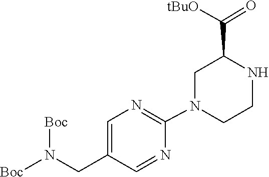 [SMILES: CC(C)(C)OC(=O)[C@@H]1CN(c2ncc(CN(C(=O)OC(C)(C)C)C(=O)OC(C)(C)C)cn2)CCN1]

Building block AV

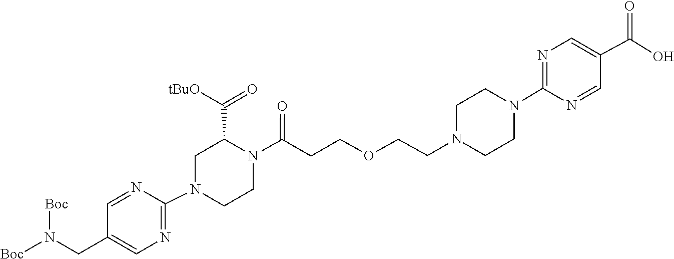 [SMILES: CC(C)(C)OC(=O)[C@H]1CN(c2ncc(CN(C(=O)OC(C)(C)C)C(=O)OC(C)(C)C)cn2)CCN1C(=O)CCOCCN1CCN(c2ncc(C(=O)O)cn2)CC1]

Building block AW

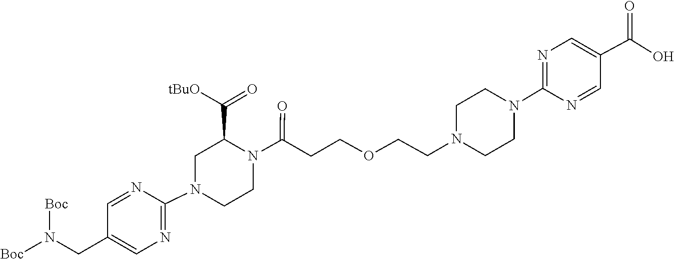 [SMILES: CC(C)(C)OC(=O)[C@@H]1CN(c2ncc(CN(C(=O)OC(C)(C)C)C(=O)OC(C)(C)C)cn2)CCN1C(=O)CCOCCN1CCN(c2ncc(C(=O)O)cn2)CC1]

Building block AX

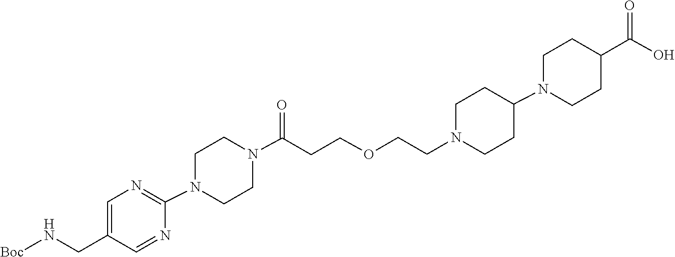 [SMILES: CC(C)(C)OC(=O)NCc1cnc(N2CCN(C(=O)CCOCCN3CCC(N4CCC(C(=O)O)CC4)CC3)CC2)nc1]

Building block AY

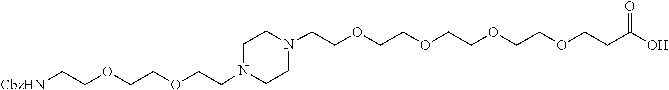 [SMILES: O=C(O)CCOCCOCCOCCOCCN1CCN(CCOCCOCCNC(=O)OCc2ccccc2)CC1]

Building block AZ

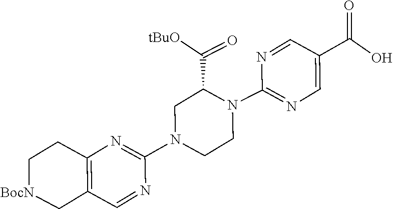 [SMILES: CC(C)(C)OC(=O)[C@H]1CN(c2ncc3c(n2)CCN(C(=O)OC(C)(C)C)C3)CCN1c1ncc(C(=O)O)cn1]

Building block BA

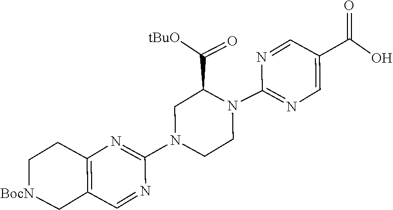 [SMILES: CC(C)(C)OC(=O)[C@@H]1CN(c2ncc3c(n2)CCN(C(=O)OC(C)(C)C)C3)CCN1c1ncc(C(=O)O)cn1]

Building block BB

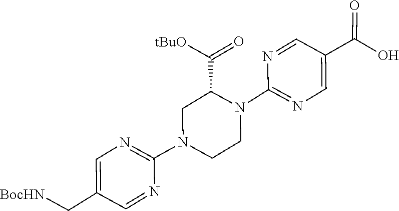 [SMILES: CC(C)(C)OC(=O)NCc1cnc(N2CCN(c3ncc(C(=O)O)cn3)[C@@H](C(=O)OC(C)(C)C)C2)nc1]

Building block BC

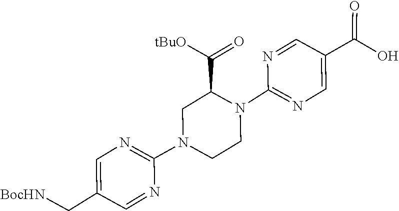 [SMILES: CC(C)(C)OC(=O)NCc1cnc(N2CCN(c3ncc(C(=O)O)cn3)[C@H](C(=O)OC(C)(C)C)C2)nc1]

Building block BD

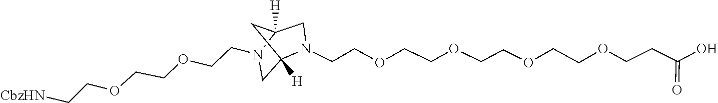

Building block BE

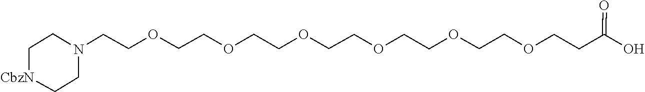 [SMILES: O=C(O)CCOCCOCCOCCOCCOCCOCCN1CCN(C(=O)OCc2ccccc2)CC1]

Building block BF

### Preparation of Active Site Inhibitor Monomers

### Monomer A. 5-(4-amino-1-(4-(aminomethyl)benzyl)-1H-pyrazolo[3,4-d]pyrimidin-3-yl)benzo[d]oxazol-2-amine trifluoroacetic acid salt

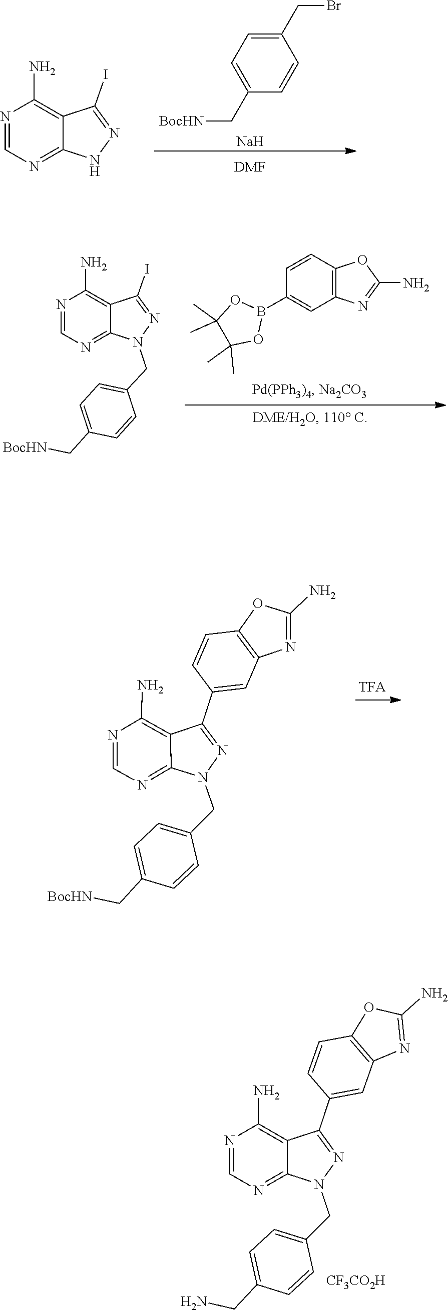

### Step 1: Synthesis of tert-butyl 4-((4-amino-3-iodo-1H-pyrazolo[3,4-d]pyrimidin-1-yl)methyl)benzylcarbamate

To a solution of 3-iodo-1H-pyrazolo[3,4-d]pyrimidin-4-amine (3.8 g, 14.56 mmol, 1.0 equiv) in DMF (20 mL) was added NaH (582.27 mg, 14.56 mmol, 60 wt. %, 1.0 equiv) at 0° C. and the reaction solution was stirred at this temperature for 30 min, then tert-butyl 4-(bromomethyl)benzylcarbamate (4.59 g, 15.29 mmol, 1.05 equiv) was added to the reaction at 0° C. and the reaction solution was stirred at room temperature for 2 h. The solution was poured into H₂O (80 mL) and the solid that precipitated out was filtered. The solid cake was washed with H₂O (2×10 mL) and then dried under reduced pressure to give tert-butyl 4-((4-amino-3-iodo-1H-pyrazolo[3,4-d]pyrimidin-1-yl)methyl)benzylcarbamate (5 g, 53% yield) as a yellow solid. LCMS (ESI) m/z: [M+Na] calcd for C₁₈H₂₁IN₆O₂: 503.07; found 503.2.

### Step 2: Synthesis of tert-butyl 4-((4-amino-3-(2-aminobenzo[d]oxazol-5-yl)-1H-pyrazolo[3,4-d]pyrimidin-1-yl)methyl)benzylcarbamate

To a bi-phasic suspension of tert-butyl 4-((4-amino-3-iodo-1H-pyrazolo[3,4-d]pyrimidin-1-yl)methyl)benzylcarbamate (5 g, 7.68 mmol, 1.0 equiv), 5-(4,4,5,5-tetramethyl-1,3,2-dioxaborolan-2-yl)benzo[d]oxazol-2-amine (2.40 g, 9.22 mmol, 1.2 equiv) and Pd(PPh₃)₄ (887.66 mg, 768.16 μmol, 0.1 equiv) in DME (100 mL) and H₂O (50 mL) was added Na₂CO₃ (1.91 g, 23.04 mmol, 3.0 equiv) at room temperature under N₂. The mixture was stirred at 110° C. for 3 h. The reaction mixture was cooled to room temperature and filtered, the filtrate was extracted by EtOAc (3×50 mL). The organic phases were combined and washed with brine (10 mL), dried over Na₂SO₄, filtered, and the filtrate was concentrated under reduced pressure to give a residue. The residue was purified by silica gel chromatography (0→20% MeOH/EtOAc) to give tert-butyl 4-((4-amino-3-(2-aminobenzo[d]oxazol-5-yl)-1H-pyrazolo[3,4-d]pyrimidin-1-yl)methyl)benzylcarbamate (4.5 g, 82% yield) as a yellow solid. LCMS (ESI) m/z: [M+H] calcd for C₂₅H₂₆N₈O₃: 487.22; found 487.2.

### Step 3: Synthesis of 5-(4-amino-1-(4-(aminomethyl)benzyl)-1H-pyrazolo[3,4-d]pyrimidin-3-yl)benzo[d]oxazol-2-amine

To a solution of tert-butyl 4-((4-amino-3-(2-aminobenzo[d]oxazol-5-yl)-1H-pyrazolo[3,4-d]pyrimidin-1-yl)methyl)benzylcarbamate (4.5 g, 6.29 mmol, 1.0 equiv) in DCM (50 mL) was added TFA (30.80 g, 270.12 mmol, 20 mL, 42.95 equiv) at 0° C. The reaction solution was stirred at room temperature for 2 h. The reaction solution was concentrated under reduced pressure to give a residue, which was dissolved in 10 mL of MeCN, then poured into MTBE (100 mL). The solid that precipitated was then filtered and the solid cake was dried under reduced pressure to give 5-[4-amino-1-[[4-(aminomethyl)phenyl]methyl]pyrazolo[3,4-d]pyrimidin-3-yl]-1,3-benzoxazol-2-amine (2.22 g, 71% yield, TFA) as a yellow solid. LCMS (ESI) m/z: [M+H] calcd for C₂₀H₁₈N₈O: 387.16; found 387.1.

### Monomer B. 2-(4-amino-1-(4-aminobutyl)-1H-pyrazolo[3,4-d]pyrimidin-3-yl)-1H-indol-6-ol trifluoroacetic acid salt

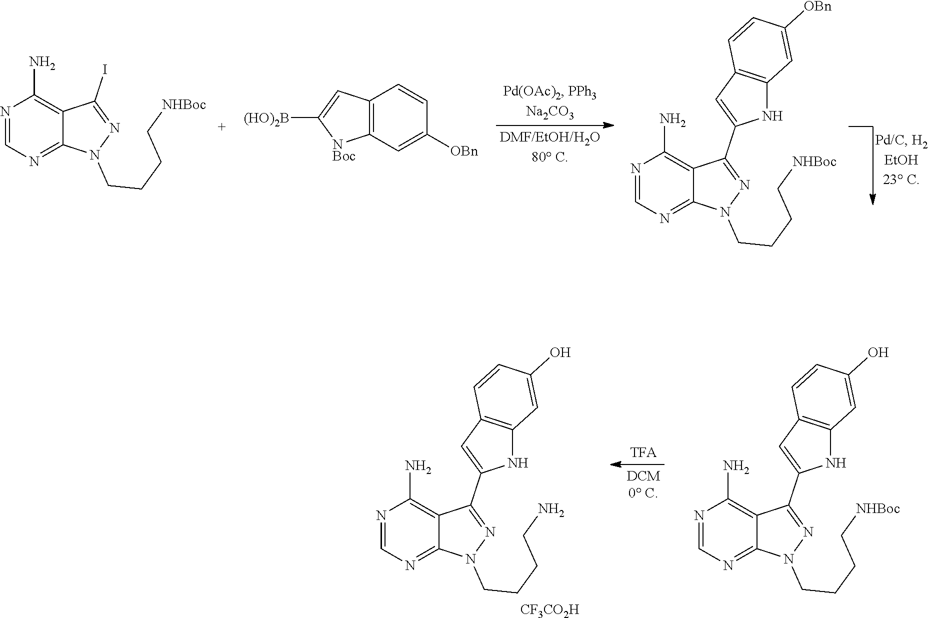 [SMILES: CC(C)(C)OC(=O)NCCCCn1nc(I)c2c(N)ncnc21 ; CC(C)(C)OC(=O)n1c(B(O)O)cc2ccc(OCc3ccccc3)cc21 ; CC(C)(C)OC(=O)NCCCCn1nc(-c2cc3ccc(OCc4ccccc4)cc3[nH]2)c2c(N)ncnc21 ; NCCCCn1nc(-c2cc3ccc(O)cc3[nH]2)c2c(N)ncnc21 ; "CF3CO2H" ; CC(C)(C)OC(=O)NCCCCn1nc(-c2cc3ccc(O)cc3[nH]2)c2c(N)ncnc21]

### Step 1: Synthesis of tert-butyl N-(4-{4-amino-3-[6-(benzyloxy)-1H-indol-2-yl]-1H-pyrazolo[3,4-d]pyrimidin-1-yl}butyl)carbamate

To a mixture of tert-butyl (4-(4-amino-3-iodo-1H-pyrazolo[3,4-d]pyrimidin-1-yl)butyl)carbamate (300 mg, 694 μmol, 1.0 equiv) and (6-(benzyloxy)-1-(tert-butoxycarbonyl)-1H-indol-2-yl)boronic acid (763 mg, 2.08 mmol, 3.0 equiv) in DMF (2.6 mL), EtOH (525 μL), and H₂O (350 μL) were added Pd(OAc)₂ (15.5 mg, 69 μmol, 0.1 equiv), triphenylphosphine (36.1 mg, 138 μmol, 0.2 equiv), and sodium carbonate (440 mg, 4.16 mmol, 6.0 equiv). The reaction was heated at 80° C. for 20 h, cooled to room temperature, and quenched with H₂O (10 mL) and EtOAc (10 mL). The mixture was transferred to a separatory funnel and the aqueous phase was extracted with EtOAc (3×20 mL). The combined organic phase was washed with sat. aq. NaCl (1×20 mL), dried over Na₂SO₄, filtered, and concentrated under reduced pressure. The crude material was purified by silica gel chromatography (20→85% EtOAc/heptane) to provide the product (201 mg, 46% yield) as an orange solid. LCMS (ESI) m/z: [M+H] calcd for C₂₉H₃₃N₇O₃: 528.27; found 528.2.

### Step 2: Synthesis of tert-butyl (4-(4-amino-3-(6-hydroxy-1H-indol-2-yl)-1H-pyrazolo[3,4-d]pyrimidin-1-yl)butyl)carbamate

To a solution of tert-butyl N-(4-{4-amino-3-[6-(benzyloxy)-1H-indol-2-yl]-1H-pyrazolo[3,4-d]pyrimidin-1-yl}butyl)carbamate (1.0 equiv) in EtOH is added Pd/C (10 mol %). The reaction is purged with H₂ and the reaction allowed to stir under an atmosphere of H₂ until consumption of starting material, as determined by LCMS. The reaction is then diluted with EtOAc, filtered over Celite, and concentrated under reduced pressure. The resultant residue is purified by silica gel chromatography to afford the desired product.

### Step 3: Synthesis of 2-(4-amino-1-(4-aminobutyl)-1H-pyrazolo[3,4-d]pyrimidin-3-yl)-1H-indol-6-ol

To a solution of tert-butyl (4-(4-amino-3-(6-hydroxy-1H-indol-2-yl)-1H-pyrazolo[3,4-d]pyrimidin-1-yl)butyl)carbamate (1.0 equiv) in anhydrous DCM is added TFA (50 equiv.) dropwise at 0° C. The reaction is stirred at 0° C. and warmed to room temperature. Once the reaction is complete, as determined by LCMS, the reaction is concentrated under reduced pressure. The residue is triturated with MeCN, then dripped into MTBE over 10 min. The supernatant is removed and the precipitate is collected by filtration under N₂ to give 2-(4-amino-1-(4-aminobutyl)-1H-pyrazolo[3,4-d]pyrimidin-3-yl)-1H-indol-6-ol.

### Monomer C. 5-(4-amino-1-((1,2,3,4-tetrahydroisoquinolin-6-yl)methyl)-1H-pyrazolo[3,4-d]pyrimidin-3-yl)benzo[d]oxazol-2-amine trifluoroacetic acid salt

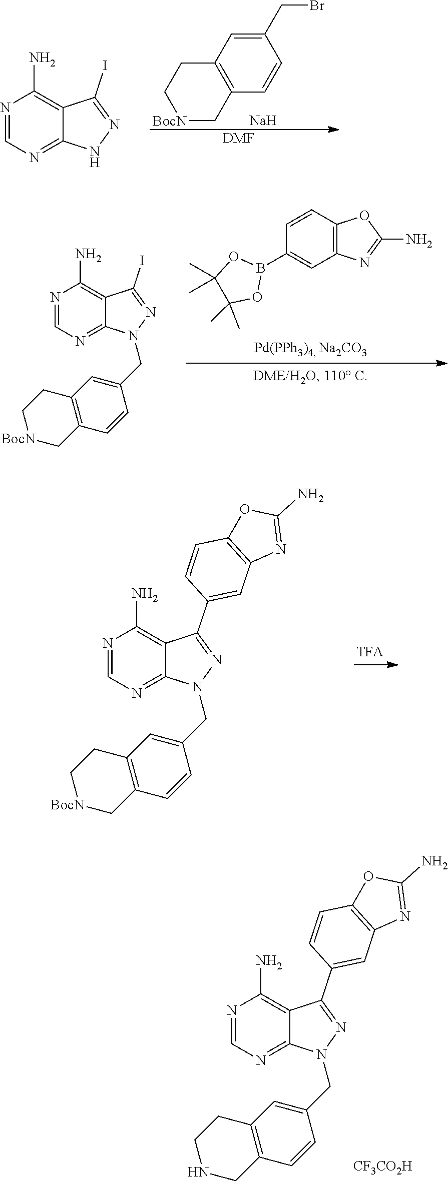

### Step 1: Synthesis of tert-butyl 6-((4-amino-3-iodo-1H-pyrazolo[3,4-d]pyrimidin-1-yl)methyl)-3,4-dihydroisoquinoline-2(1H)-carboxylate

To a suspension of 3-iodo-1H-pyrazolo[3,4-d]pyrimidin-4-amine (5 g, 19.16 mmol, 1.0 equiv) in DMF (50.0 mL) was added NaH (766.22 mg, 19.16 mmol, 60 wt. %, 1.0 equiv) at 4° C. The mixture was stirred at 4° C. for 30 min. To the reaction mixture was added tert-butyl 6-(bromomethyl)-3,4-dihydroisoquinoline-2(1H)-carboxylate (6.87 g, 21.07 mmol, 1.1 equiv) in DMF (30 mL) at 4° C. The mixture was stirred at room temperature for 2 h. The mixture was then cooled to 4° C. and H₂O (400 mL) was added and the mixture was stirred for 30 min. The resulting precipitate was collected by filtration to give crude tert-butyl 6-((4-amino-3-iodo-1H-pyrazolo[3,4-d]pyrimidin-1-yl)methyl)-3,4-dihydroisoquinoline-2(1H)-carboxylate (9.7 g, 76% yield) as a light yellow solid. The crude product was used for the next step directly.

### Step 2: Synthesis of tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate

To a bi-phasic suspension of tert-butyl 6-((4-amino-3-iodo-1H-pyrazolo[3,4-d]pyrimidin-1-yl)methyl)-3,4-dihydroisoquinoline-2(1H)-carboxylate (9.7 g, 14.63 mmol, 1.0 equiv), 5-(4,4,5,5-tetramethyl-1,3,2-dioxaborolan-2-yl)benzo[d]oxazol-2-amine (4.57 g, 17.55 mmol, 1.2 equiv), and Na₂CO₃ (7.75 g, 73.14 mmol, 5.0 equiv) in DME (120.0 mL) and H₂O (60 mL) was added Pd(PPh₃)₄ (1.69 g, 1.46 mmol, 0.1 equiv) at room temperature under N₂. The mixture was stirred at 110° C. for 3 h. The reaction mixture was then cooled to room temperature and partitioned between EtOAc (100 mL) and H₂O (100 mL). The aqueous layer was separated and extracted with EtOAc (2×60 mL). The organic layers were combined, washed with brine (80 mL) and dried over anhydrous Na₂SO₄, filtered and the filtrate was concentrated under reduced pressure. The residue was purified by silica gel chromatography (1→100% EtOAc/petroleum ether, then 20→50% MeOH/EtOAc) to afford tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate (4.5 g, 58% yield) as a light yellow solid.

### Step 3: Synthesis of 5-(4-amino-1-((1,2,3,4-tetrahydroisoquinolin-6-yl)methyl)-1H-pyrazolo[3,4-d]pyramidin-3-yl)benzo[d]oxazol-2-amine

To neat TFA (32.5 mL, 438.97 mmol, 50.0 equiv) was added tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate (4.5 g, 8.78 mmol, 1.0 equiv) at room temperature. The mixture was stirred for 30 min and then concentrated under reduced pressure. The oily residue was triturated with MeCN (8 mL), then dripped into MTBE (350 mL) over 10 min. The supernatant was removed and then the precipitate was collected by filtration under N₂ to give 5-(4-amino-1-((1,2,3,4-tetrahydroisoquinolin-6-yl)methyl)-1H-pyrazolo[3,4-d]pyrimidin-3-yl)benzo[d]oxazol-2-amine (5.72 g, over 100% yield, TFA) as a light pink solid. LCMS (ESI) m/z: [M+H] calcd for C₂₂H₂₀N₈O: 413.18; found 413.2.

### Monomer D. 2-(4-amino-1-(4-aminobutyl)-1H-pyrazolo[3,4-d]pyrimidin-3-yl)-1H-indol-7-ol trifluoroacetic acid salt

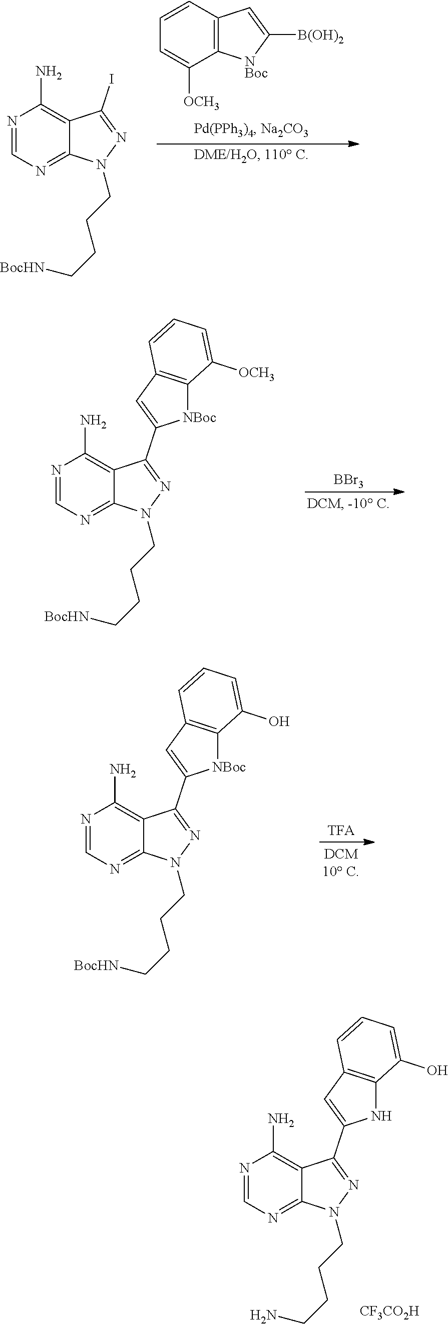

### Step 1: Synthesis of tert-butyl 2-(4-amino-1-(4-((tert-butoxycarbonyl)amino)butyl)-1H-pyrazolo[3,4-d]pyrimidin-3-yl)-7-methoxy-1H-indole-1-carboxylate

To a mixture of tert-butyl (4-(4-amino-3-iodo-1H-pyrazolo[3,4-d]pyrimidin-1-yl)butyl)carbamate (1.0 equiv) and (1-(tert-butoxycarbonyl)-7-methoxy-1H-indol-2-yl)boronic acid (3.0 equiv) in DME and H₂O is added Pd(PPh₃)₄ (0.1 equiv) and sodium carbonate (6.0 equiv). The reaction is heated at 80° C. until completion, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 2: Synthesis of tert-butyl 2-(4-amino-1-(4-((tert-butoxycarbonyl)amino)butyl)-1H-pyrazolo[3,4-d]pyrimidin-3-yl)-7-hydroxy-1H-indole-1-carboxylate

To a solution of tert-butyl 2-(4-amino-1-(4-((tert-butoxycarbonyl)amino)butyl)-1H-pyrazolo[3,4-d]pyrimidin-3-yl)-7-methoxy-1H-indole-1-carboxylate (1.0 equiv) in DCM at −10° C. is added BBr₃ (2.0 equiv). The reaction is allowed to stir until consumption of starting material, as determined by LCMS. The reaction is quenched by slow addition of sat. aq. NaHCO₃, transferred to a separatory funnel and the mixture is extracted with DCM. The organic phase is washed with sat. aq. NaCl, dried over Na₂SO₄, filtered, and concentrated under reduced pressure. The desired product is isolated after chromatography on silica gel.

### Step 3: Synthesis of 2-(4-amino-1-(4-aminobutyl)-1H-pyrazolo[3,4-d]pyrimidin-3-yl)-1H-indol-7-ol

To a solution of tert-butyl 2-(4-amino-1-(4-((tert-butoxycarbonyl)amino)butyl)-1H-pyrazolo[3,4-d]pyrimidin-3-yl)-7-hydroxy-1H-indole-1-carboxylate (1.0 equiv) in DCM at 0° C. is added TFA dropwise. The reaction is stirred at 0° C. and warmed to room temperature. Once the reaction is complete, as determined by LCMS, the reaction is concentrated under reduced pressure. The residue is triturated with MeCN, then dripped into MTBE over 10 min. The supernatant is removed and the precipitate is collected by filtration under N₂ to give 2-(4-amino-1-(4-aminobutyl)-1H-pyrazolo[3,4-d]pyrimidin-3-yl)-1H-indol-7-ol.

### Monomer E. 5-(4-amino-1-(piperidin-4-ylmethyl)-1H-pyrazolo[3,4-d]pyrimidin-3-yl)benzo[d]oxazol-2-amine trifluoroacetic acid salt

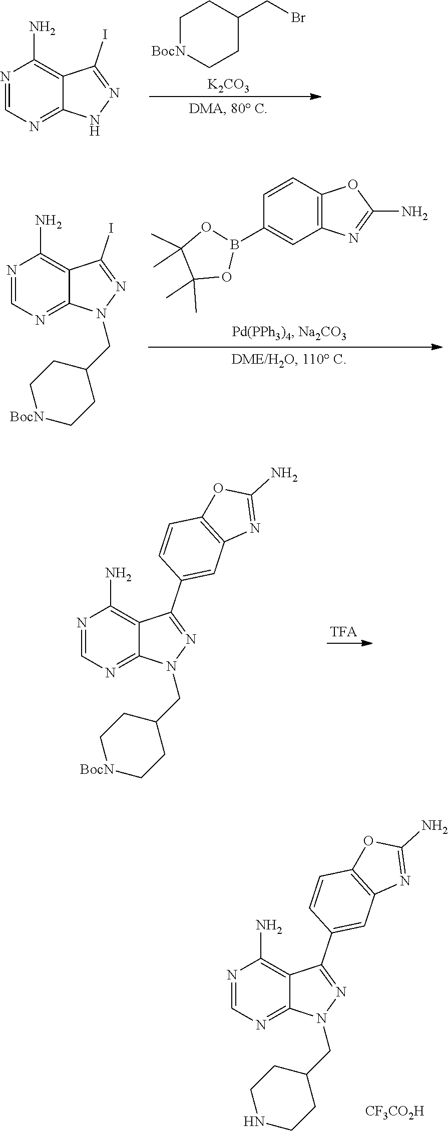

### Step 1: Synthesis of tert-butyl 4-((4-amino-3-iodo-1H-pyrazolo[3,4-d]pyrimidin-1-yl)methyl)piperidine-1-carboxylate

To a solution of 3-iodo-1H-pyrazolo[3,4-d]pyrimidin-4-amine (3 g, 11.49 mmol, 1.0 equiv) in DMA (30 mL) was added tert-butyl 4-(bromomethyl)piperidine-1-carboxylate (3.36 g, 12.07 mmol, 1.05 equiv) and K₂CO₃ (4.77 g, 34.48 mmol, 3.0 equiv), then the reaction was stirred at 80° C. for 3 h. The reaction mixture was filtered to remove K₂CO₃ and the filtrate was poured into H₂O (200 mL). A solid precipitated was then filtered to give tert-butyl 4-((4-amino-3-iodo-1H-pyrazolo[3,4-d]pyrimidin-1-yl)methyl)piperidine-1-carboxylate (3 g, 57% yield) as a light yellow solid. LCMS (ESI) m/z: [M+H] calcd for C₁₆H₂₃IN₆O₂: 459.10; found 459.1.

### Step 2: Synthesis of tert-butyl 4-((4-amino-3-(2-aminobenzo[d]oxazol-5-yl)-1H-pyrazolo[3,4-d]pyrimidin-1-yl)methyl)piperidine-1-carboxylate

To a bi-phasic suspension of tert-butyl 4-((4-amino-3-iodo-1H-pyrazolo[3,4-d]pyrimidin-1-yl)methyl)piperidine-1-carboxylate (3 g, 6.55 mmol, 1.0 equiv) and 5-(4,4,5,5-tetramethyl-1,3,2-dioxaborolan-2-yl)benzo[d]oxazol-2-amine (2.04 g, 7.86 mmol, 1.2 equiv) and Na₂CO₃ (3.47 g, 32.73 mmol, 5.0 equiv) in DME (60 mL) and H₂O (30 mL) was added Pd(PPh₃)₄ (756.43 mg, 654.60 μmol, 0.1 equiv) at room temperature under N₂. The mixture was stirred at 110° C. for 3 h and the two batches were combined together. The reaction mixture was cooled and partitioned between EtOAc (500 mL) and H₂O (500 mL). The aqueous layer was separated and extracted with EtOAc (3×300 mL). All the organic layers were combined, washed with brine (20 mL), dried over anhydrous Na₂SO₄, filtered, and the filtrate was concentrated under reduced pressure to give tert-butyl 4-((4-amino-3-(2-aminobenzo[d]oxazol-5-yl)-1H-pyrazolo[3,4-d]pyrimidin-1-yl)methyl)piperidine-1-carboxylate (4.5 g, 74% yield) as a yellow solid. LCMS (ESI) m/z: [M+H] calcd for C₂₃H₂₈N₈O₃: 465.24; found 465.2.

### Step 3: Synthesis of 5-(4-amino-1-(piperidin-4-ylmethyl)-1H-pyrazolo[3,4-d]pyrimidin-3-yl)benzo[d]oxazol-2-amine

A solution of tert-butyl 4-((4-amino-3-(2-aminobenzo[d]oxazol-5-yl)-1H-pyrazolo[3,4-d]pyrimidin-1-yl)methyl)piperidine-1-carboxylate (2.5 g, 5.38 mmol, 1.0 equiv) in TFA (25 mL) was stirred at room temperature for 30 min. The reaction solution was concentrated under reduced pressure to remove TFA. The residue was added to MTBE (400 mL) and a solid precipitated, which was then filtered to give 5-(4-amino-1-(piperidin-4-ylmethyl)-1H-pyrazolo[3,4-d]pyrimidin-3-yl)benzo[d]oxazol-2-amine (2.7 g, over 100% yield, TFA) as a yellow solid. LCMS (ESI) m/z: [M+H] calcd for C₁₈H₂₀N₈O: 365.18; found 365.1.

### Monomer F. 2-(4-amino-1-(4-aminobutyl)-1H-pyrazolo[3,4-d]pyrimidin-3-yl)-1H-indol-5-ol trifluoroacetic acid salt

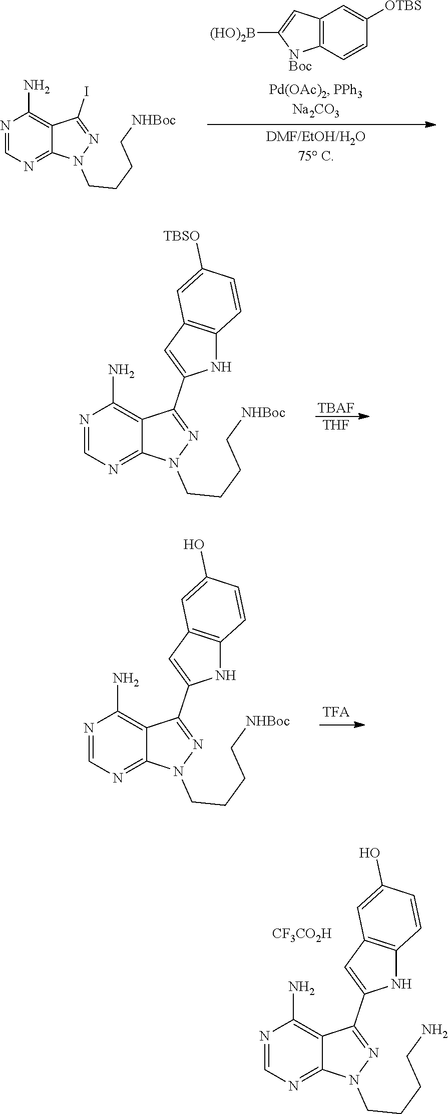

### Step 1: Synthesis of tert-butyl (4-(4-amino-3-(5-((tert-butyldimethylsilyl)oxy)-1H-indol-2-yl)-1H-pyrazolo[3,4-d]pyrimidin-1-yl)butyl)carbamate

To a solution of tert-butyl (4-(4-amino-3-iodo-1H-pyrazolo[3,4-d]pyrimidin-1-yl)butyl)carbamate (1.0 g, 2.31 mmol, 1.0 equiv) in dioxane (10.5 mL) and H₂O (3.5 mL) was added (1-(tert-butoxycarbonyl)-5-((tert-butyldimethylsilyl)oxy)-1H-indol-2-yl)boronic acid (1.54 g, 2.78 mmol, 1.2 equiv), K₃PO₄ (1.47 g, 6.94 mmol, 3.0 equiv), Pd₂(dba)₃ (211.84 mg, 231.34 μmol, 0.1 equiv), and SPhos (189.95 mg, 462.69 μmol, 0.2 equiv) at room temperature under N₂. The sealed tube was heated at 150° C. for 20 min in a microwave. This was repeated for 9 additional batches. The 10 batches were combined and the reaction mixture was cooled and partitioned between EtOAc (60 mL) and H₂O (80 mL). The aqueous layer was separated and extracted with EtOAc (2×50 mL). The organic layers were combined, washed with brine (60 mL) and dried over anhydrous Na₂SO₄. The suspension was filtered and the filtrate was concentrated under reduced pressure. The crude material was purified by silica gel chromatography (1→75% EtOAc/petroleum ether). The desired fractions were combined and evaporated under reduced pressure to give tert-butyl (4-(4-amino-3-(5-((tert-butyldimethylsilyl)oxy)-1H-indol-2-yl)-1H-pyrazolo[3,4-d]pyrimidin-1-yl)butyl)carbamate (10 g, 60% yield) as a light yellow solid.

### Step 2: Synthesis of tert-butyl (4-(4-amino-3-(5-hydroxy-1H-indol-2-yl)-1H-pyrazolo[3,4-d]pyrimidin-1-yl)butyl)carbamate

To a mixture of tert-butyl (4-(4-amino-3-(5-((tert-butyldimethylsilyl)oxy)-1H-indol-2-yl)-1H-pyrazolo[3,4-d]pyrimidin-1-yl)butyl)carbamate (10 g, 18.12 mmol, 1.0 equiv) in THF (100 mL) was added TBAF·3H₂O (1 M, 54.37 mL, 3.0 equiv) in one portion at room temperature under N₂. The mixture was stirred for 1 h and then H₂O (100 mL) was added to the reaction mixture. The layers were separated and the aqueous phase was extracted with EtOAc (2×80 mL). The combined organic phase was washed with brine (100 mL), dried with anhydrous Na₂SO₄, filtered and concentrated under reduced pressure. The residue was purified by silica gel chromatography (1→67% EtOAc/petroleum ether) to afford tert-butyl (4-(4-amino-3-(5-hydroxy-1H-indol-2-yl)-1H-pyrazolo[3,4-d]pyrimidin-1-yl)butyl)carbamate (7 g, 87% yield) as a light pink solid.

### Step 3: Synthesis of 2-[4-amino-1-(4-aminobutyl)pyrazolo[3,4-d]pyrimidin-3-yl]-1H-indol-5-ol

To TFA (50.0 mL, 675.26 mmol, 38.9 equiv) was added tert-butyl (4-(4-amino-3-(5-hydroxy-1H-indol-2-yl)-1H-pyrazolo[3,4-d]pyrimidin-1-yl)butyl)carbamate (7.6 g, 17.37 mmol, 1.0 equiv) at room temperature. The mixture was stirred for 40 min and was then concentrated under reduced pressure. The oily residue was triturated with MeCN (20 mL), then added dropwise into MTBE (300 mL) for 10 min. The supernatant was removed and then the precipitate was collected by filtration under N₂ to give 2-[4-amino-1-(4-aminobutyl)pyrazolo[3,4-d]pyrimidin-3-yl]-1H-indol-5-ol (7.79 g, 91% yield, TFA) as light yellow solid. LCMS (ESI) m/z: [M+H] calcd for C₁₇H₁₉N₇O: 338.17; found 338.2.

### Monomer G. 5-(4-amino-1-(azetidin-3-ylmethyl)-1H-pyrazolo[3,4-d]pyrimidin-3-yl)benzo[d]oxazol-2-amine trifluoroacetic acid salt

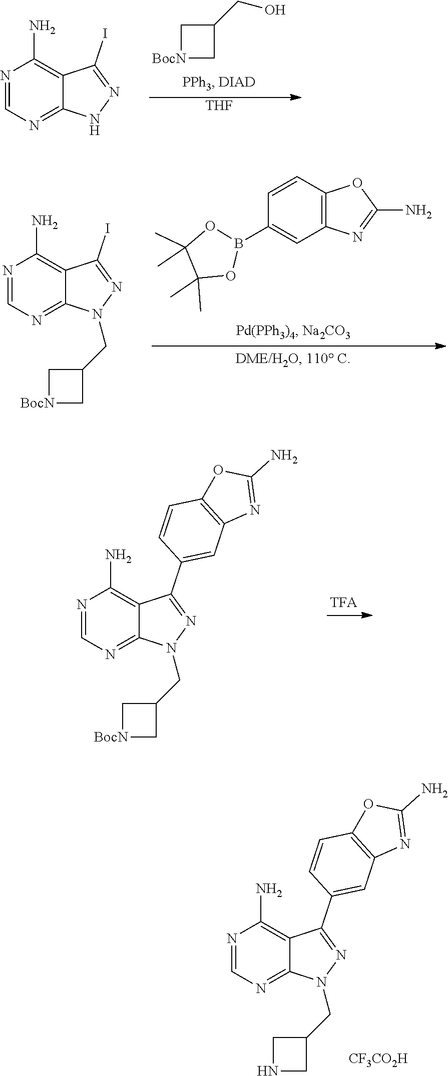

### Step 1: Synthesis of tert-butyl 3-((4-amino-3-iodo-1H-pyrazolo[3,4-d]pyrimidin-1-yl) methyl)azetidine-1-carboxylate

To a solution of 3-iodo-1H-pyrazolo[3,4-d]pyrimidin-4-amine (4 g, 15.32 mmol, 1.0 equiv), tert-butyl 3-(hydroxymethyl)azetidine-1-carboxylate (3.01 g, 16.09 mmol, 1.05 equiv) and PPh₃ (6.03 g, 22.99 mmol, 1.5 equiv) in THF (80 mL) cooled to 0° C. was added DIAD (4.47 mL, 22.99 mmol, 1.5 equiv), dropwise. After the addition was complete, the reaction was stirred at room temperature for 14 h. The reaction was poured into H₂O (200 mL) and then extracted with EtOAc (3×50 mL). The organic layers were combined and washed with brine (2×50 mL). The organic phase was dried over Na₂SO₄, filtered, the filtrate was concentrated under reduced pressure to give a residue. The residue was purified by silica gel chromatography (0→100% EtOAc/petroleum ether) to give tert-butyl 3-((4-amino-3-iodo-1H-pyrazolo[3,4-d]pyrimidin-1-yl)methyl) azetidine-1-carboxylate (4.2 g, 64% yield) as a white solid. LCMS (ESI) m/z: [M+H] calcd for C₁₄H₁₉IN₆O₆: 431.07; found 431.0.

### Step 2: Synthesis of tert-butyl 3-((4-amino-3-(2-aminobenzo[d]oxazol-5-yl)-1H-pyrazolo [3,4-d]pyrimidin-1-yl)methyl)azetidine-1-carboxylate

To a bi-phasic suspension of tert-butyl 3-((4-amino-3-iodo-1H-pyrazolo[3,4-d]pyrimidin-1-yl) methyl)azetidine-1-carboxylate (4 g, 9.30 mmol, 1.0 equiv), 5-(4,4,5,5-tetramethyl-1,3,2-dioxaborolan-2-yl)benzo[d]oxazol-2-amine (2.90 g, 11.16 mmol, 1.2 equiv) and Na₂CO₃ (4.93 g, 46.49 mmol, 5.0 equiv) in DME (100 mL) and H₂O (50 mL) was added Pd(PPh₃)₄ (1.07 g, 929.71 μmol, 0.1 equiv) at room temperature under N₂. The mixture was stirred at 110° C. for 3 h. The reaction mixture was then cooled to room temperature and filtered, and the filtrate was extracted by EtOAc (3×50 mL). The organic layers were combined and washed with brine (10 mL), dried over Na₂SO₄, filtered and the filtrate was concentrated under reduced pressure to give a residue. The residue was purified by silica gel chromatography (0→20% MeOH/EtOAc) to give tert-butyl 3-((4-amino-3-(2-aminobenzo[d]oxazol-5-yl)-1H-pyrazolo[3,4-d]pyrimidin-1-yl)methyl)azetidine-1-carboxylate (3.5 g, 80% yield) as a yellow solid. LCMS (ESI) m/z: [M+H] calcd for C₂₁H₂₄N₈O₃: 437.20; found 437.2.

### Step 3: Synthesis of 5-(4-amino-1-(azetidin-3-ylmethyl)-1H-pyrazolo[3,4-d]pyrimidin-3-yl)benzo[d]oxazol-2-amine

To a solution of tert-butyl 3-((4-amino-3-(2-aminobenzo[d]oxazol-5-yl)-1H-pyrazolo[3,4-d]pyrimidin-1-yl)methyl)azetidine-1-carboxylate (3.29 g, 6.87 mmol, 1.0 equiv) in DCM (20 mL) was added TFA (7.50 mL, 101.30 mmol, 14.7 equiv) at 0° C. The reaction was warmed to room temperature and stirred for 2 h. The reaction solution was concentrated under reduced pressure to give a residue. The residue was dissolved in MeCN (6 mL) and then poured into MTBE (80 mL). A solid precipitated, which was filtered and the solid cake was dried under reduced pressure to give 5-[4-amino-1-(azetidin-3-ylmethyl)pyrazolo[3,4-d]pyrimidin-3-yl]-1,3-benzoxazol-2-amine (4.34 g, over 100% yield, TFA) as a yellow solid. LCMS (ESI) m/z: [M+H] calcd for C₁₆H₁₆N₈O: 337.15; found 337.1.

### Monomer H. 5-(4-amino-1-(4-aminobutyl)-1H-pyrazolo[3,4-d]pyrimidin-3-yl)benzo[d]-oxazol-2-amine trifluoroacetic acid salt

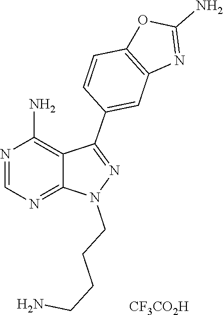 [SMILES: NCCCCn1nc(-c2ccc3oc(N)nc3c2)c2c(N)ncnc21 ; "CF3CO2H"]

This monomer was synthesized following the procedures outlined in Nature 2015, 534, 272-276, which is incorporated by reference in its entirety.

### Monomer I. 5-(4-amino-1-(pyrrolidin-3-ylmethyl)-1H-pyrazolo[3,4-d]pyrimidin-3-yl)benzo[d]oxazol-2-amine trifluoroacetic acid salt

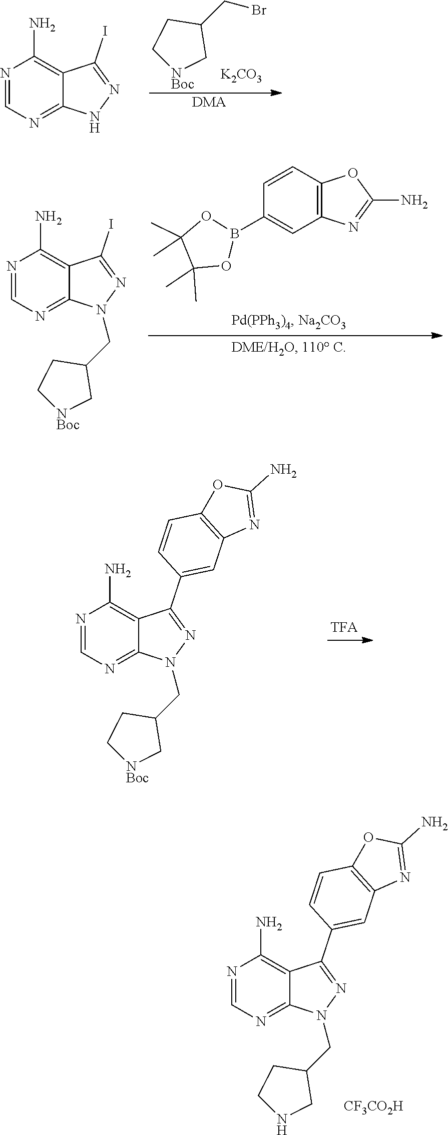

### Step 1: Synthesis of tert-butyl 3-((4-amino-3-iodo-1H-pyrazolo[3,4-d]pyrimidin-1-yl) methyl)pyrrolidine-1-carboxylate

A suspension of 3-iodo-1H-pyrazolo[3,4-d]pyrimidin-4-amine (4.5 g, 17.24 mmol, 1.0 equiv), tert-butyl 3-(bromomethyl)pyrrolidine-1-carboxylate (4.78 g, 18.10 mmol, 1.05 equiv) and K₂CO₃ (7.15 g, 51.72 mmol, 3.0 equiv) in DMA (40 mL) was heated to 85° C. The reaction was stirred at 85° C. for 3 h, at which point the solution was cooled to room temperature. Then, H₂O (80 mL) was added to the reaction, and a solid precipitated out. The mixture was filtered, and the solid cake was washed with H₂O (2×40 mL), and then dried under reduced pressure to give tert-butyl 3-((4-amino-3-iodo-1H-pyrazolo[3,4-d]pyrimidin-1-yl) methyl)pyrrolidine-1-carboxylate (6 g, 78% yield) as a yellow solid. LCMS (ESI) m/z: [M+H] calcd for C₁₅H₂₁IN₆O₂: 445.08; found 445.1.

### Step 2: Synthesis of tert-butyl 3-[[4-amino-3-(2-amino-1,3-benzoxazol-5-yl)pyrazolo[3,4-d]pyrimidin-1-yl]methyl]pyrrolidine-1-carboxylate

To a bi-phasic suspension of tert-butyl 3-((4-amino-3-iodo-1H-pyrazolo[3,4-d]pyrimidin-1-yl) methyl)pyrrolidine-1-carboxylate (4 g, 9.00 mmol, 1.0 equiv), 5-(4,4,5,5-tetramethyl-1,3,2-dioxaborolan-2-yl)benzo[d]oxazol-2-amine (2.81 g, 10.80 mmol, 1.2 equiv) and Na₂CO₃ (4.77 g, 45.02 mmol, 5.0 equiv) in DME (120 mL) and H₂O (60 mL) was added Pd(PPh₃)₄ (1.04 g, 900.35 μmol, 0.1 equiv) at room temperature under N₂. The mixture was stirred at 110° C. for 3 h. The reaction mixture was cooled to room temperature and filtered and the filtrate was extracted with EtOAc (3×50 mL). The organic phases were combined and washed with brine (50 mL), dried over Na₂SO₄, filtered and concentrated under reduced pressure to give a residue. The residue was purified by silica gel chromatography (0→20% MeOH/EtOAc) to give tert-butyl 3-((4-amino-3-(2-aminobenzo[d]oxazol-5-yl)-1H-pyrazolo[3,4-d]pyrimidin-1-yl) methyl)pyrrolidine-1-carboxylate (3 g, 64% yield) as a yellow solid. LCMS (ESI) m/z: [M+H] calcd for C₂₂H₂₆N₈O₃: 451.21, found 451.2.

### Step 3: Synthesis of 5-(4-amino-1-(pyrrolidin-3-ylmethyl)-1H-pyrazolo[3,4-d]pyrimidin-3-yl)benzo[d]oxazol-2-amine

To a solution of tert-butyl 3-((4-amino-3-(2-aminobenzo[d]oxazol-5-yl)-1H-pyrazolo[3,4-d]pyrimidin-1-yl)methyl)pyrrolidine-1-carboxylate (3 g, 6.66 mmol, 1.0 equiv) in DCM (40 mL) was added TFA (20 mL) at 0° C., dropwise. The reaction mixture was warmed to room temperature and stirred for 2 h. The reaction solution was then concentrated under reduced pressure to give a residue. The residue was dissolved in MeCN (4 mL), then poured into MTBE (100 mL), and a solid precipitated out. The solid was filtered and the cake was dried under reduced pressure to give 5-(4-amino-1-(pyrrolidin-3-ylmethyl)-1H-pyrazolo[3,4-d]pyrimidin-3-yl)benzo[d]oxazol-2-amine (4.00 g, over 100% yield, TFA) as a yellow solid. LCMS (ESI) m/z: [M+H] calcd for C₁₇H₁₈N₈O: 351.17; found 351.2.

### Monomer J. 1-(4-aminobutyl)-3-(7-methoxy-1H-indol-2-yl)-1H-pyrazolo[3,4-d]pyrimidin-4-aminetrifluoroacetic acid salt

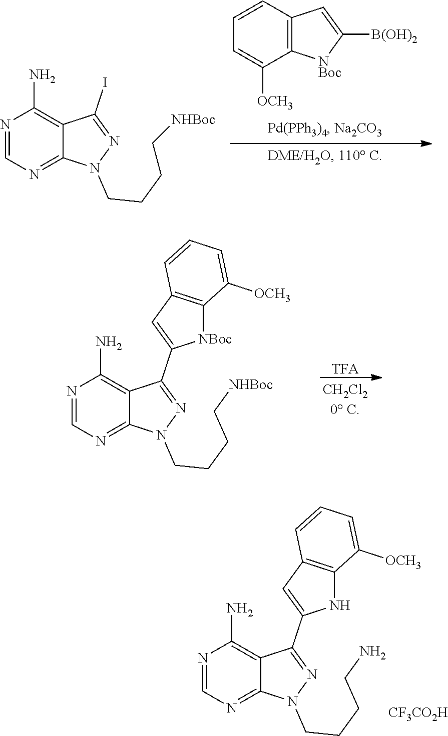 [SMILES: CC(C)(C)OC(=O)NCCCCn1nc(I)c2c(N)ncnc21 ; COc1cccc2cc(B(O)O)n(C(=O)OC(C)(C)C)c12 ; COc1cccc2cc(-c3nn(CCCCNC(=O)OC(C)(C)C)c4ncnc(N)c34)n(C(=O)OC(C)(C)C)c12 ; COc1cccc2cc(-c3nn(CCCCN)c4ncnc(N)c34)[nH]c12 ; "CF3CO2H" ; "TFA"]

### Step 1: Synthesis of tert-butyl 2-(4-amino-1-(4-((tert-butoxycarbonyl)amino)butyl)-1H-pyrazolo[3,4-d]pyrimidin-3-yl)-7-methoxy-1H-indole-1-carboxylate

To a mixture of tert-butyl (4-(4-amino-3-iodo-1H-pyrazolo[3,4-d]pyrimidin-1-yl)butyl)carbamate (1.0 equiv) and (1-(tert-butoxycarbonyl)-7-methoxy-1H-indol-2-yl)boronic acid (3.0 equiv) in DME and H₂O is added Pd(PPh₃)₄ (0.1 equiv) and sodium carbonate (6.0 equiv). The reaction is heated at 80° C. until completion, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 2: Synthesis of 1-(4-aminobutyl)-3-(7-methoxy-1H-indol-2-yl)-1H-pyrazolo[3,4-d]pyrimidin-4-amine

To a solution of tert-butyl 2-(4-amino-1-(4-((tert-butoxycarbonyl)amino)butyl)-1H-pyrazolo[3,4-d]pyrimidin-3-yl)-7-hydroxy-1H-indole-1-carboxylate (1.0 equiv) in DCM at 0° C. is added TFA dropwise. The reaction is stirred at 0° C. and warmed to room temperature. Once the reaction is complete, as determined by LCMS, the reaction is concentrated under reduced pressure. The residue is triturated with MeCN, then dripped into MTBE over 10 min. The supernatant is removed and the precipitate is collected by filtration under N₂ to give 1-(4-aminobutyl)-3-(7-methoxy-1H-indol-2-yl)-1H-pyrazolo[3,4-d]pyrimidin-4-amine.

### Monomer K. Synthesis of 1-(4-aminobutyl)-1H-pyrazolo[3,4-d]pyrimidin-4-amine trifluoroacetic acid salt

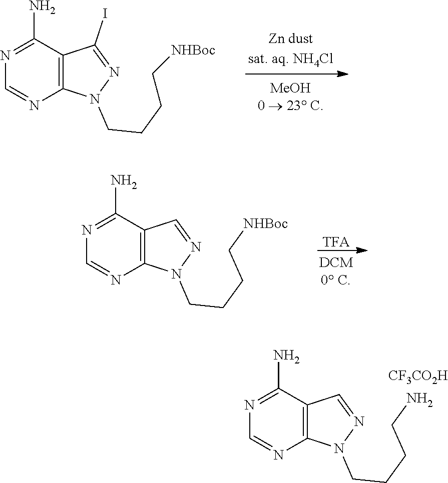 [SMILES: CC(C)(C)OC(=O)NCCCCn1nc(I)c2c(N)ncnc21 ; CC(C)(C)OC(=O)NCCCCn1ncc2c(N)ncnc21 ; NCCCCn1ncc2c(N)ncnc21 ; "CF3CO2H" ; "TFA"]

### Step 1: Synthesis of tert-butyl (4-(4-amino-1H-pyrazolo[3,4-d]pyrimidin-1-yl)butyl)carbamate

To a mixture of tert-butyl (4-(4-amino-3-iodo-1H-pyrazolo[3,4-d]pyrimidin-1-yl)butyl)carbamate (300 mg, 694 μmol, 1.0 equiv) in MeOH (14 mL) at 0° C. was added zinc dust (226 mg, 3.46 mmol, 5.0 equiv). Sat. aq. NH₄Cl (14 mL) was added to the reaction mixture and the reaction was warmed to room temperature and stirred for 18 h. The reaction was quenched by EtOAc (40 mL) and H₂O (10 mL) and the mixture was transferred to a separatory funnel. The aqueous phase was extracted with EtOAc (3×20 mL) and the combined organic phases were washed with sat. aq. NaHCO₃ (15 mL), dried over Na₂SO₄, filtered, and concentrated under reduced pressure to provide the product (210 mg, 99% yield) as a light yellow solid that was used without further purification. LCMS (ESI) m/z: [M+H] calcd for C₁₄H₂₂N₆O₂: 307.19; found 307.1.

### Step 2: Synthesis of 1-(4-aminobutyl)-1H-pyrazolo[3,4-d]pyrimidin-4-amine

To a solution of tert-butyl (4-(4-amino-1H-pyrazolo[3,4-d]pyrimidin-1-yl)butyl)carbamate (210 mg, 691 μmol) in DCM (3.5 mL) at 0° C. was added TFA (3.5 mL), dropwise. After 3 h, the reaction was warmed to room temperature and concentrated under reduced pressure to provide the trifluoroacetate salt of the product (220 mg, 99% yield) as a brown oil, which was used without further purification. LCMS (ESI) m/z: [M+H] calcd for C₉H₁₄N₆: 207.13; found 207.1.

### Monomer L. 1-[4-(piperazin-1-yl)-3-(trifluoromethyl)phenyl]-9-(quinolin-3-yl)-1H,2H-benzo[h]1,6-naphthyridin-2-one

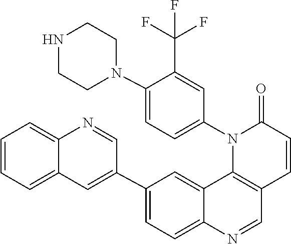 [SMILES: O=c1ccc2cnc3ccc(-c4cnc5ccccc5c4)cc3c2n1-c1ccc(N2CCNCC2)c(C(F)(F)F)c1]

The preparation of this monomer has been previously reported in the literature. See the following references: i) Liu, Qingsong; Chang, Jae Won; Wang, Jinhua; Kang, Seong A.; Thoreen, Carson C.; Markhard, Andrew; Hur, Wooyoung; Zhang, Jianming; Sim, Taebo; Sabatini, David M.; et al From Journal of Medicinal Chemistry (2010), 53(19), 7146-7155. ii) Gray, Nathanael; Chang, Jae Won; Zhang, Jianming; Thoreen, Carson C.; Kang, Seong Woo Anthony; Sabatini, David M.; Liu, Qingsong From PCT Int. Appl. (2010), WO 2010044885A2, which are incorporated by reference in their entirety.

### Monomer M. 5-(1-(4-aminobutyl)-4-(dimethylamino)-1H-pyrazolo[3,4-d]pyrimidin-3-yl)benzo[d]oxazol-2-amine trifluoroacetic acid salt

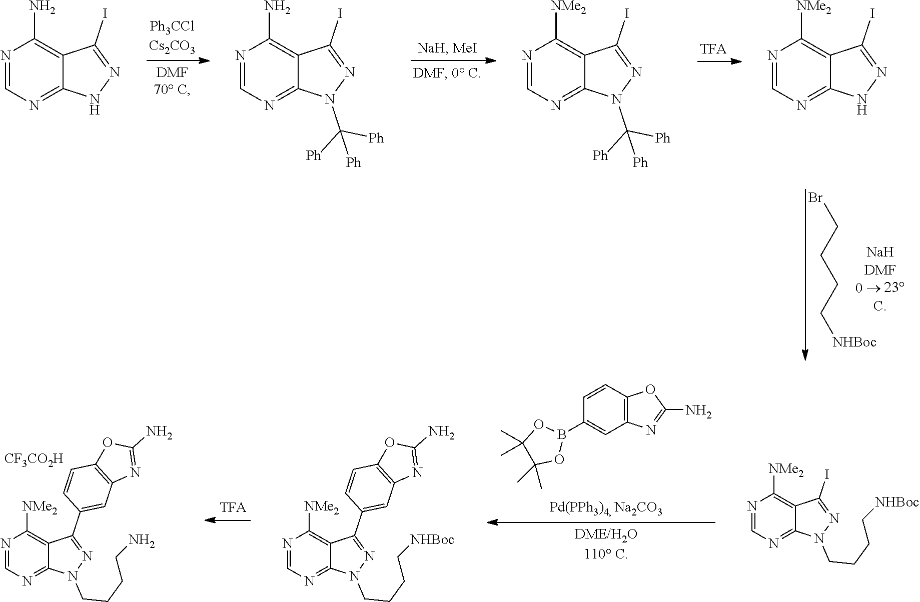

### Step 1: Synthesis of 3-iodo-1-trityl-1H-pyrazolo[3,4-d]pyrimidin-4-amine

A suspension of 3-iodo-1H-pyrazolo[3,4-d]pyrimidin-4-amine (10.5 g, 40.23 mmol, 1.0 equiv) in DMF (170.0 mL) was treated with Cs₂CO₃ (19.7 g, 60.34 mmol, 1.5 equiv) and [chloro(diphenyl)methyl]benzene (13.5 g, 48.27 mmol, 1.2 equiv) at room temperature. The reaction mixture was stirred at 70° C. for 4 h under a nitrogen atmosphere. The reaction mixture was added to H₂O (1200 mL). The precipitate was filtered and washed with H₂O. The residue was purified by silica gel chromatography (0→60% EtOAc/petroleum ether) to afford 3-iodo-1-trityl-1H-pyrazolo[3,4-d]pyrimidin-4-amine (15.40 g, 73.5% yield) as a white solid.

### Step 2: Synthesis of 3-iodo-N,N-dimethyl-1-trityl-1H-pyrazolo[3,4-d]pyrimidin-4-amine

To a suspension of NaH (2.98 g, 74.50 mmol, 60 wt. %, 2.5 equiv) in DMF (150 mL) was added the solution of 3-iodo-1-trityl-1H-pyrazolo[3,4-d]pyrimidin-4-amine (15.0 g, 29.80 mmol, 1.0 equiv) in DMF (50 mL) at 0° C. The mixture was stirred at 0° C. for 10 min. To the reaction mixture was then added iodomethane (16.92 g, 119.20 mmol, 7.42 mL, 4.0 equiv) at 0° C. The mixture was stirred at room temperature for 2 h, at which point H₂O (1400 mL) was added at 0° C. The mixture was stirred for an additional 10 min at 0° C. The resulting precipitate was collected by filtration to give crude product, which was purified by silica gel chromatography (1%→25% EtOAc/petroleum ether) twice to afford 3-iodo-N,N-dimethyl-1-trityl-1H-pyrazolo[3,4-d]pyrimidin-4-amine (9.0 g, 89% yield) as a white solid.

### Step 3: Synthesis of 3-iodo-N,N-dimethyl-1H-pyrazolo[3,4-d]pyrimidin-4-amine

To a cooled solution of TFA (19.1 mL, 258.1 mmol, 15.0 equiv) in DCM (100.0 mL) was added 3-iodo-N,N-dimethyl-1-trityl-1H-pyrazolo[3,4-d]pyrimidin-4-amine (9.10 g, 17.12 mmol, 1.0 equiv) at 4° C. The mixture was stirred at room temperature for 1 h. The residue was poured into H₂O (100 mL) and the aqueous phase was extracted with DCM (2×50 mL). To the aqueous phase was then added a saturated aqueous solution of NaHCO₃ until the solution was pH 8. The resulting precipitate was collected by filtration to give 3-iodo-N,N-dimethyl-1H-pyrazolo[3,4-d]pyrimidin-4-amine (3.40 g, 68.7% yield) as a white solid.

### Step 4: Synthesis of tert-butyl (4-(4-(dimethylamino)-3-iodo-1H-pyrazolo[3,4-d]pyrimidin-1-yl)butyl)carbamate

To a suspension of 3-iodo-N,N-dimethyl-1H-pyrazolo[3,4-d]pyrimidin-4-amine (1.7 g, 5.88 mmol, 1.0 equiv) in DMF (20 mL) was added NaH (247 mg, 6.17 mmol, 60 wt. %, 1.05 equiv) at 4° C. The mixture was stirred at 4° C. for 30 min. To the reaction mixture was then added tert-butyl N-(4-bromobutyl)carbamate (2.22 g, 8.82 mmol, 1.81 mL, 1.5 equiv) in DMF (10 mL) at 4° C. The mixture was stirred at room temperature for 2 h. To the mixture was then added H₂O (100 mL) at 4° C. The mixture was stirred for an additional 30 min at 4° C. and the resulting precipitate was collected by filtration to give crude product. The residue was purified by silica gel chromatography (0→75% EtOAc/petroleum ether) to afford tert-butyl(4-(4-(dimethylamino)-3-iodo-1H-pyrazolo[3,4-d]pyrimidin-1-yl)butyl)carbamate (2.0 g, 56% yield) as a white solid.

### Step 5: Synthesis of tert-butyl (4-(3-(2-aminobenzo[d]oxazol-5-yl)-4-(dimethylamino)-1H-pyrazolo[3,4-d]pyrimidin-1-yl)butyl)carbamate

To a bi-phasic suspension of tert-butyl (4-(4-(dimethylamino)-3-iodo-1H-pyrazolo[3,4-d]pyrimidin-1-yl)butyl)carbamate (4.0 g, 8.69 mmol, 1.0 equiv), 5-(4,4,5,5-tetramethyl-1,3,2-dioxaborolan-2-yl)benzo[d]oxazol-2-amine (3.4 g, 13.03 mmol, 1.5 equiv), and Na₂CO₃ (4.6 g, 43.45 mmol, 5.0 equiv) in DME (80.0 mL) and H₂O (40.0 mL) was added Pd(PPh₃)₄ (1.0 g, 868.98 μmol, 0.1 equiv) at room temperature under N₂. The mixture was stirred at 110° C. for 3 h. The reaction mixture was then cooled and partitioned between EtOAc (300 mL) and H₂O (600 mL). The aqueous layer was separated and extracted with EtOAc (2×100 mL). The organic layers were combined, washed with brine (2×60 mL) and dried over anhydrous Na₂SO₄, filtered, and concentrated under reduced pressure. The crude material was purified by silica gel column chromatography (50% EtOAc/hexanes followed by 20% MeOH/EtOAc). The desired fractions were combined and concentrated under reduced pressure to give tert-butyl (4-(3-(2-aminobenzo[d]oxazol-5-yl)-4-(dimethylamino)-1H-pyrazolo[3,4-d]pyramidin-1-yl)butyl)carbamate (3.2 g, 78.9% yield) as a light brown solid.

### Step 6: Synthesis of 5-(1-(4-aminobutyl)-4-(dimethylamino)-1H-pyrazolo[3,4-d]pyrimidin-3-yl)benzo[d]oxazol-2-amine

To TFA (20.82 mL, 281.27 mmol, 36.5 equiv) was added tert-butyl (4-(3-(2-aminobenzo[d]oxazol-5-yl)-4-(dimethylamino)-1H-pyrazolo[3,4-d]pyrimidin-1-yl)butyl)carbamate (3.6 g, 7.72 mmol, 1.0 equiv) at room temperature. The mixture was stirred for 30 min, at which point the mixture was concentrated under reduced pressure. The oily residue was triturated with MeCN (8 mL) and MTBE (60 mL) for 10 min. The supernatant was removed and then the precipitate was collected by filtration under N₂ to give 5-(1-(4-aminobutyl)-4-(dimethylamino)-1H-pyrazolo[3,4-d]pyrimidin-3-yl)benzo[d]oxazol-2-amine (4.0 g, crude, TFA) as a light brown solid.

To 1 M NaOH (107.2 mL, 14.7 equiv) was added 5-(1-(4-aminobutyl)-4-(dimethylamino)-1H-pyrazolo[3,4-d]pyrimidin-3-yl)benzo[d]oxazol-2-amine (3.5 g, crude, TFA) at room temperature. The mixture was stirred for 10 min and then the aqueous phase was extracted with DCM (3×50 mL). The combined organic phase was washed with brine (50 mL), dried with anhydrous Na₂SO₄, filtered and concentrated under reduced pressure. TFA (539.37 μL, 7.28 mmol, 1.0 equiv) was added and concentrated under reduced pressure. MeCN (10 mL) was then added, followed by MTBE (150 mL). The resulting precipitate was collected by filtration to give 5-(1-(4-aminobutyl)-4-(dimethylamino)-1H-pyrazolo[3,4-d]pyrimidin-3-yl)benzo[d]oxazol-2-amine (1.3 g, 36.6% yield, TFA) as a light brown product. LCMS (ESI) m/z: [M+H] calcd for C₁₈H₂₂N₈O: 367.19; found 367.1.

### Monomer N. 6-(4-amino-1-(4-aminobutyl)-1H-pyrazolo[3,4-d]pyrimidin-3-yl)benzo-[d]isoxazol-3-amine trifluoroacetic acid salt

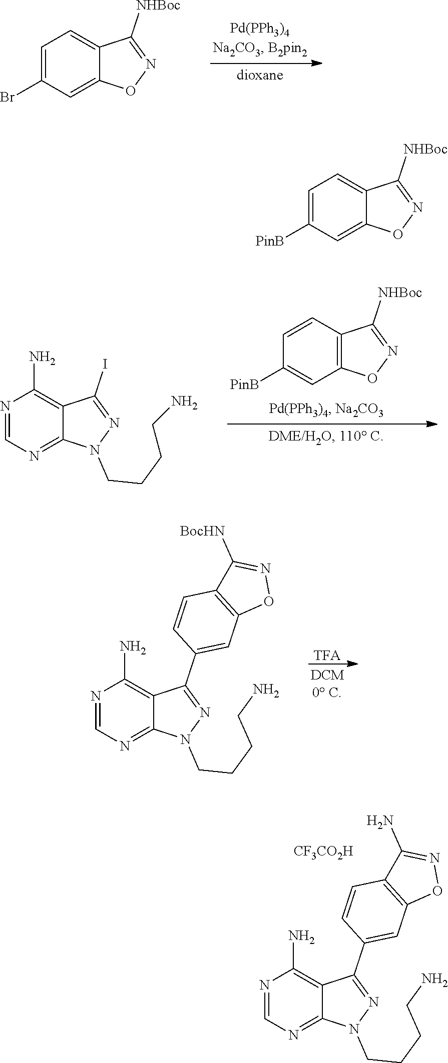

### Step 1: Synthesis of tert-butyl (6-(4,4,5,5-tetramethyl-1,3,2-dioxaborolan-2-yl)benzo[d]isoxazol-3-yl)carbamate

To a solution of tert-butyl (6-bromobenzo[d]isoxazol-3-yl)carbamate (1.0 equiv) in dioxane is added Pd(PPh₃)₄ (0.1 equiv), sodium carbonate (6.0 equiv), and bis(pinacolato)diboron (3.0 equiv). The reaction mixture is stirred and heated until completion, as determined by LCMS and TLC analysis. The reaction is cooled to room temperature, quenched with sat. aq. NaHCO₃, and the mixture transferred to a separatory funnel. The aqueous phase is extracted with EtOAc and the organic phase is washed with sat. aq. NaCl, dried over Na₂SO₄, filtered, and concentrated under reduced pressure. The desired product is isolated after purification by silica gel chromatography.

### Step 2: Synthesis of tert-butyl (4-(4-amino-3-(3-((tert-butoxycarbonyl)amino)benzo[d]isoxazol-6-yl)-1H-pyrazolo[3,4-d]pyrimidin-1-yl)butyl)carbamate

To a mixture of tert-butyl (4-(4-amino-3-iodo-1H-pyrazolo[3,4-d]pyrimidin-1-yl)butyl)carbamate (1.0 equiv) and tert-butyl (6-(4,4,5,5-tetramethyl-1,3,2-dioxaborolan-2-yl)benzo[d]isoxazol-3-yl)carbamate (3.0 equiv) in DME and H₂O is added Pd(PPh₃)₄ (0.1 equiv) and sodium carbonate (6.0 equiv). The reaction is heated at 80° C. until completion, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 3: Synthesis of 6-(4-amino-1-(4-aminobutyl)-1H-pyrazolo[3,4-d]pyrimidin-3-yl)benzo-[d]isoxazol-3-amine

To a solution of tert-butyl (4-(4-amino-3-(3-((tert-butoxycarbonyl)amino)benzo[d]isoxazol-6-yl)-1H-pyrazolo[3,4-d]pyrimidin-1-yl)butyl)carbamate (1.0 equiv) in DCM at 0° C. is added TFA, dropwise. The reaction is stirred at 0° C. and warmed to room temperature. Once the reaction is complete, as determined by LCMS, the reaction is concentrated under reduced pressure. The residue is triturated with MeCN, then added dropwise into MTBE over 10 min. The supernatant is removed and the precipitate is collected by filtration under N₂ to give 6-(4-amino-1-(4-aminobutyl)-1H-pyrazolo[3,4-d]pyrimidin-3-yl)benzo-[d]isoxazol-3-amine.

### Monomer O. 4-(5-(4-morpholino-1-(1-(pyridin-3-ylmethyl)piperidin-4-yl)-1H-pyrazolo[3,4-d]pyrimidin-6-yl)-1H-indol-1-yl)butan-1-amine trifluoroacetic acid salt

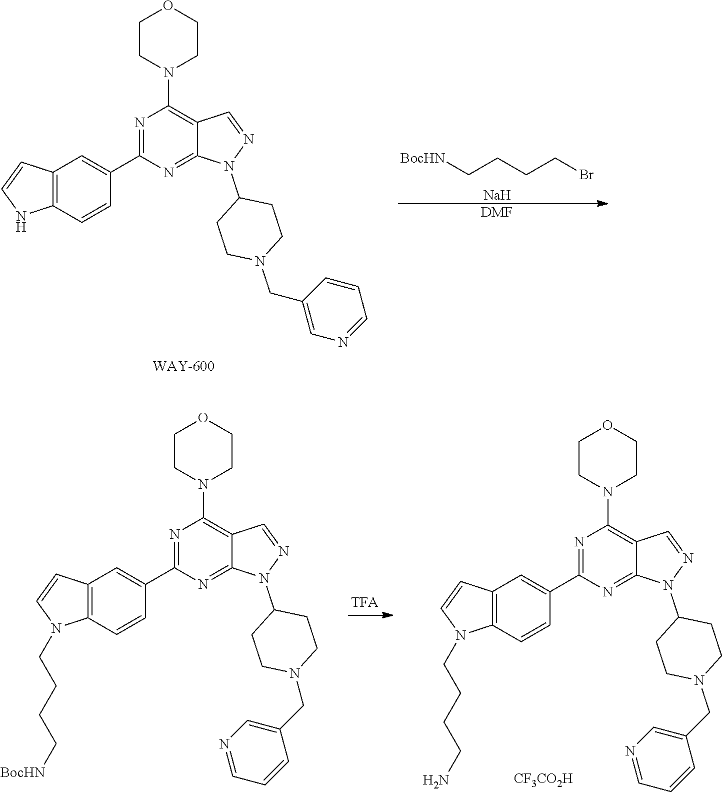

The synthesis of this monomer proceeds by alkylation of WAY-600 (CAS #1062159-35-6) with tert-butyl (4-bromobutyl)carbamate under basic conditions, followed by Boc-deprotection using TFA to produce the TFA salt.

Reference for preparation of WAY-600: Discovery of Potent and Selective Inhibitors of the Mammalian Target of Rapamycin (mTOR) Kinase: Nowak, P.; Cole, D. C.; Brooijmans, N.; Bursavich, M. G.; Curran, K. J.; Ellingboe, J. W.; Gibbons, J. J.; Hollander, I.; Hu, Y.; Kaplan, J.; Malwitz, D. J.; Toral-Barza, L.; Verheijen, J. C.; Zask, A.; Zhang, W.-G.; Yu, K. 2009; Journal of Medicinal Chemistry Volume 52, Issue 22, 7081-89, which is incorporated by reference in its entirety.

### Monomer P. 2-(4-(8-(6-(aminomethyl)quinolin-3-yl)-3-methyl-2-oxo-2,3-dihydro-1H-imidazo[4,5-c]quinolin-1-yl)phenyl)-2-methylpropanenitrile trifluoroacetic acid salt

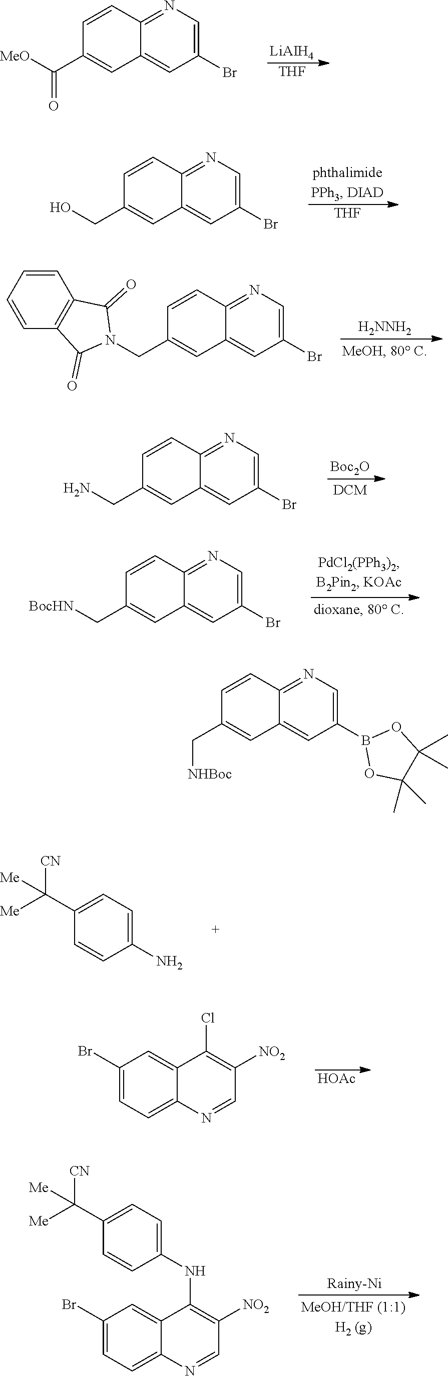

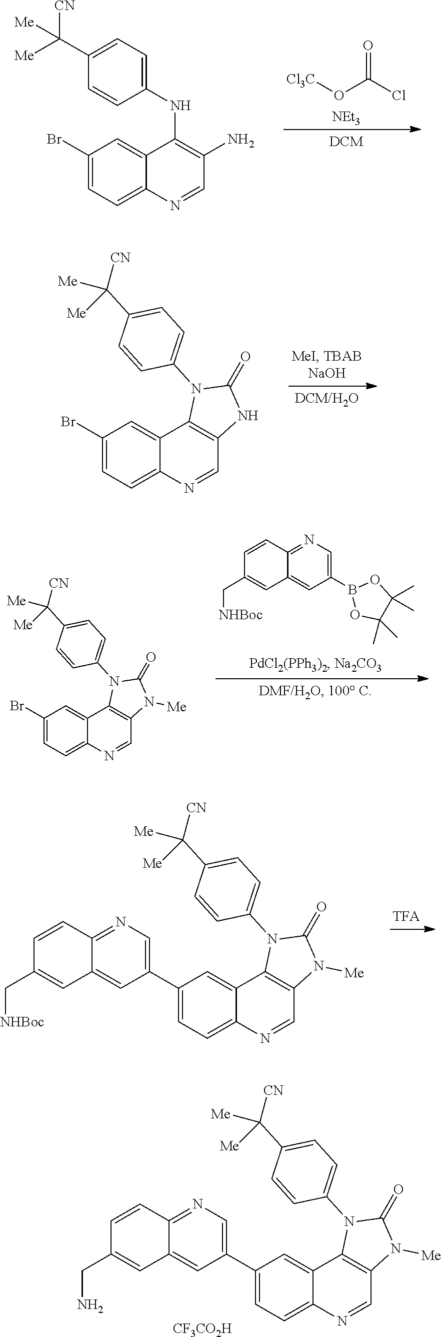

The synthesis of this monomer proceeds first by synthesis of the Suzuki reaction coupling partner (3-(4,4,5,5-tetramethyl-1,3,2-dioxaborolane)quinolin-6-yl)-N-boc-methanamine starting from methyl 3-bromoquinoline-6-carboxylate. Reduction of the methyl ester with lithium aluminum hydride followed by Mitsunobu reaction with phthalimide and hydrazine cleavage provides the benzylic amine. Protection of the benzylic amine with di-tert-butyl dicarbonate followed by a Miyaura borylation reaction provides (3-(4,4,5,5-tetramethyl-1,3,2-dioxaborolane)quinolin-6-yl)-N-boc-methanamine.

An S_{N}Ar reaction of 2-(4-aminophenyl)-2-methylpropanenitrile with 6-bromo-4-chloro-3-nitroquinoline provides the substituted amino-nitro-pyridine. Reduction of the nitro group with Raney-Ni under a hydrogen atmosphere followed by cyclization with trichloromethyl chloroformate provides the aryl-substituted urea. Substitution of the free N—H of the urea with methyl iodide mediated by tetrabutylammonium bromide and sodium hydroxide followed by Suzuki coupling of (3-(4,4,5,5-tetramethyl-1,3,2-dioxaborolane)quinolin-6-yl)-N-boc-methanamine and then Boc-deprotection using TFA produces the TFA salt.

Reference for preparation of 2-[4-(8-bromo-3-methyl-2-oxo-2,3-dihydro-imidazo [4,5-c]quinolin-1-yl)-phenyl]-2-methyl-propionitrile: Vannucchi, A. M.; Bogani, C.; Bartalucci, N. 2016. JAK PI3K/mTOR combination therapy. U.S. Pat. No. 9,358,229. Novartis Pharma AG, Incyte Corporation, which is incorporated by reference in its entirety.

### Monomer Q. 8-(6-methoxypyridin-3-yl)-3-methyl-1-[4-(piperazin-1-yl)-3-(trifluoromethyl)phenyl]-1H,2H,3H-imidazo[4,5-c]quinolin-2-one

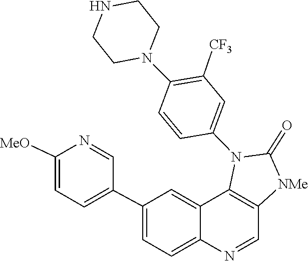 [SMILES: COc1ccc(-c2ccc3ncc4c(c3c2)n(-c2ccc(N3CCNCC3)c(C(F)(F)F)c2)c(=O)n4C)cn1]

This monomer is a commercially available chemical known as BGT226(CAS #1245537-68-1). At the time this application was prepared, it was available for purchase from several vendors as the free amine.

### Monomer R. 3-(4-amino-1-(4-aminobutyl)-1H-pyrazolo[3,4-d]pyrimidin-3-yl)-N-(4,5-dihydrothiazol-2-yl)benzamide trifluoroacetic acid salt

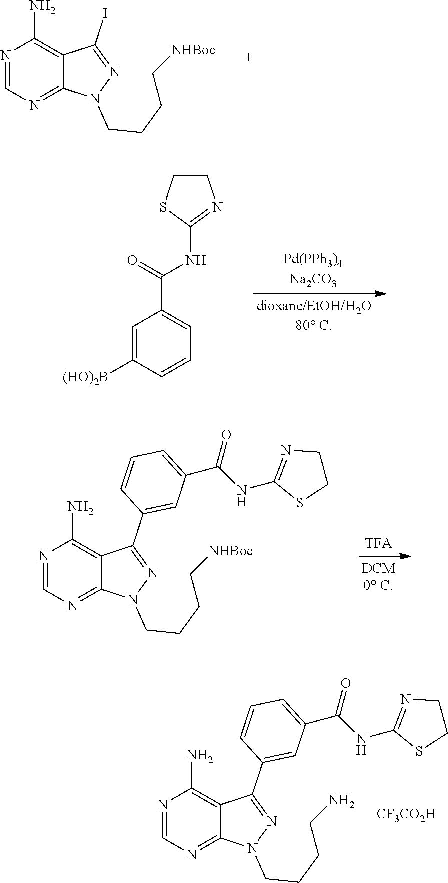 [SMILES: CC(C)(C)OC(=O)NCCCCn1nc(I)c2c(N)ncnc21 ; O=C(NC1=NCCS1)c1cccc(B(O)O)c1 ; CC(C)(C)OC(=O)NCCCCn1nc(-c2cccc(C(=O)NC3=NCCS3)c2)c2c(N)ncnc21 ; NCCCCn1nc(-c2cccc(C(=O)NC3=NCCS3)c2)c2c(N)ncnc21 ; "CF3CO2H"]

### Step 1: Synthesis of tert-butyl (4-(4-amino-3-(3-((4,5-dihydrothiazol-2-yl)carbamoyl)phenyl)-1H-pyrazolo[3,4-d]pyrimidin-1-yl)butyl)carbamate

To a solution of (3-((4,5-dihydrothiazol-2-yl)carbamoyl)phenyl)boronic acid (500 mg, 1.15 mmol, 1.0 equiv) and tert-butyl (4-(4-amino-3-iodo-1H-pyrazolo[3,4-d]pyrimidin-1-yl)butyl)carbamate (575 mg, 2.30 mmol, 2.0 equiv) in dioxane (19.1 mL), EtOH (3.8 mL), and H₂O (2.3 mL) was added Pd(PPh₃)₄ (265 mg, 230 μmol, 0.2 equiv) and sodium carbonate (730 mg, 6.89 mmol, 6.0 equiv). The reaction mixture was sonicated until formation of a clear, yellow solution, which was subsequently heated at 80° C. for 14 h. The reaction was then diluted with sat. aq. NaCl (30 mL) and the mixture transferred to a separatory funnel. The aqueous phase was extracted with DCM (3×25 mL). The combined organic phases were dried over Na₂SO₄, filtered, and concentrated under reduced pressure. The desired product was isolated as a yellow solid (324 mg, 53% yield) after silica gel chromatography (0→15% MeOH/DCM). LCMS (ESI) m/z: [M+H] calcd for C₂₄H₃₀N₈O₃S: 511.22; found 511.2.

### Step 2: Synthesis of 3-(4-amino-1-(4-aminobutyl)-1H-pyrazolo[3,4-d]pyrimidin-3-yl)-N-(4,5-dihydrothiazol-2-yl)benzamide

To a solution of tert-butyl (4-(4-amino-3-(3-((4,5-dihydrothiazol-2-yl)carbamoyl)phenyl)-1H-pyrazolo[3,4-d]pyrimidin-1-yl)butyl)carbamate (324 mg, 614 μmol) in DCM (4.1 mL) at 0° C. was added TFA (1.5 mL), dropwise. After 1 h, the reaction was warmed to room temperature and concentrated under reduced pressure to provide the trifluoroacetate salt of the product as a yellow solid (320 mg, 99% yield). Used without further purification. LCMS (ESI) m/z: [M+H] calcd for C₁₉H₂₂N₈OS: 411.16; found 411.1.

### Monomer S. 2-(5-(4-morpholino-1-(1-(pyridin-3-ylmethyl)piperidin-4-yl)-1H-pyrazolo[3,4-d]pyrimidin-6-yl)-1H-indol-3-yl)ethan-1-amine

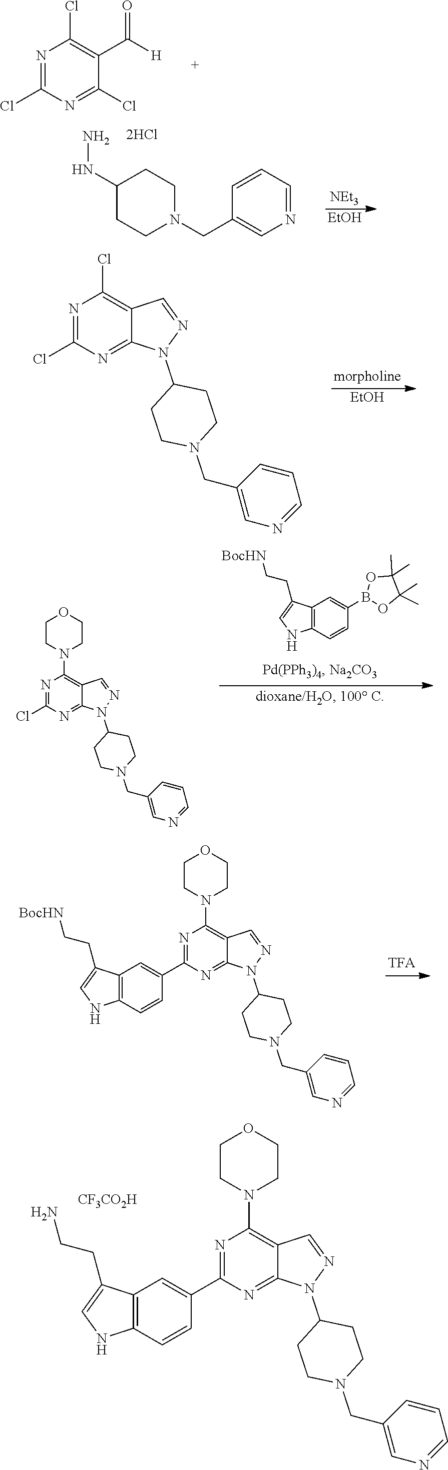

The synthesis of this monomer proceeds by condensation of 2,4,6-trichloropyrimidine-5-carbaldehyde with 3-((4-hydrazineylpiperidin-1-yl)methyl)pyridine hydrochloride. Reaction of the product with morpholine followed by a Suzuki reaction with boronic ester gives the Boc-protected amine. Final deprotection with TFA gives the monomer. This synthesis route follows closely to the reported preparation of highly related structures in the following references: i) Nowak, Pawel; Cole, Derek C.; Brooijmans, Natasja; Curran, Kevin J.; Ellingboe, John W.; Gibbons, James J.; Hollander, Irwin; Hu, Yong Bo; Kaplan, Joshua; Malwitz, David J.; et al From Journal of Medicinal Chemistry (2009), 52(22), 7081-7089. ii) Zask, Arie; Nowak, Pawel Wojciech; Verheijen, Jeroen; Curran, Kevin J.; Kaplan, Joshua; Malwitz, David; Bursavich, Matthew Gregory; Cole, Derek Cecil; Ayral-Kaloustian, Semiramis; Yu, Ker; et al From PCT Int. Appl. (2008), WO 2008115974 A2 20080925, which are incorporated by reference in their entirety.

### Monomer T. 1-(4-aminobutyl)-3-iodo-1H-pyrazolo[3,4-d]pyrimidin-4-amine trifluoroacetic acid salt

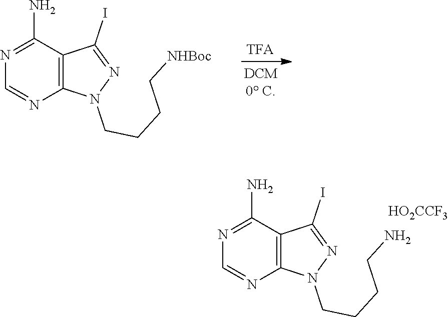

To a mixture of tert-butyl (4-(4-amino-3-iodo-1H-pyrazolo[3,4-d]pyrimidin-1-yl)butyl)carbamate (496 mg, 1.14 mmol, 1.0 equiv) in DCM (5.7 mL) at 0° C. was added TFA (1.5 mL) dropwise. The reaction was allowed to stir at 0° C. for 1 h, at which time the reaction was concentrated under reduced pressure to provide a yellow solid (505 mg, 99% yield) which was taken on without further purification. LCMS (ESI) m/z: [M+H] calcd for C₉H₁₃IN₆: 333.02; found 332.9.

### Monomer U. 5-(4-amino-1-(4-(methylamino)butyl)-1H-pyrazolo[3,4-d]pyrimidin-3-yl)benzo[d]oxazol-2-amine trifluoroacetic acid salt

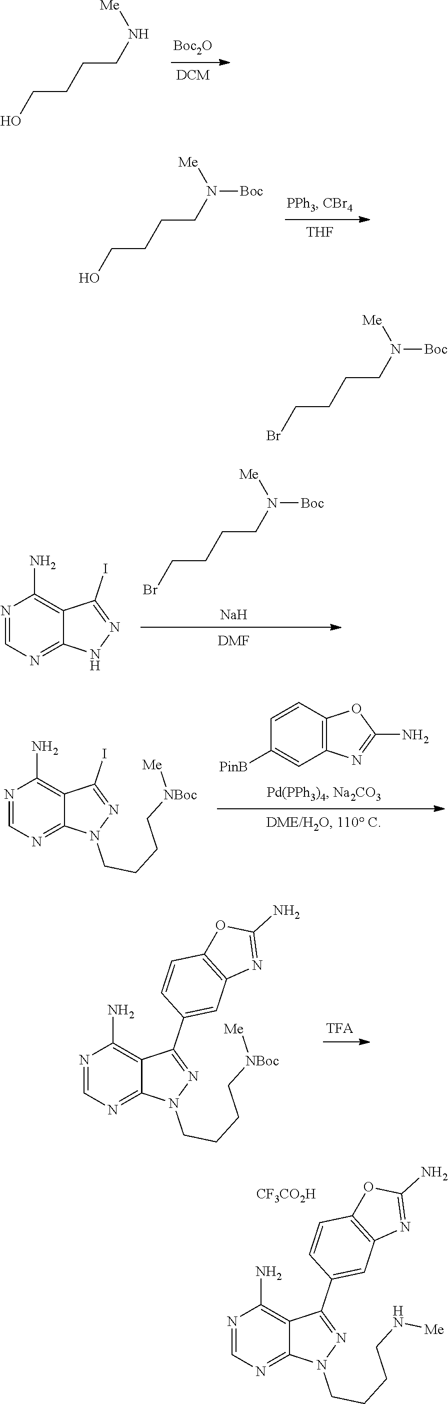

### Step 1: Synthesis of tert-butyl (4-hydroxybutyl)(methyl)carbamate

To a solution of 4-(methylamino)butan-1-ol (0.5 g, 4.85 mmol, 104.2 mL, 1.0 equiv) in DCM (10 mL) at room temperature was added Boc₂O (1.06 g, 4.85 mmol, 1.11 mL, 1.0 equiv). The mixture was stirred for 3 h at room temperature and then the mixture was concentrated under reduced pressure at 30° C. The residue was purified by silica gel chromatography (100/1 to 3/1 petroleum ether/EtOAc) to afford tert-butyl (4-hydroxybutyl)(methyl)carbamate (0.9 g, 91.4% yield) as a colorless oil.

### Step 2: Synthesis of tert-butyl (4-bromobutyl)(methyl)carbamate

To a solution of tert-butyl (4-hydroxybutyl)(methyl)carbamate (0.9 g, 4.43 mmol, 1.0 equiv) in THF (20 mL) at room temperature was added PPh₃ (2.21 g, 8.41 mmol, 1.9 equiv) and CBr₄ (2.79 g, 8.41 mmol, 1.9 equiv). The mixture was stirred for 1 h and then the reaction mixture was filtered and concentrated. The residue was purified by silica gel chromatography (1/0 to 4/1 petroleum ether/EtOAc) to afford tert-butyl (4-bromobutyl)(methyl) carbamate (1.1 g, 93.3% yield) as a colorless oil.

### Step 3: Synthesis of tert-butyl (4-(4-amino-3-iodo-1H-pyrazolo[3,4-d]pyrimidin-1-yl) butyl) (methyl)carbamate

To a suspension of 3-iodo-1H-pyrazolo[3,4-d]pyrimidin-4-amine (0.9 g, 3.45 mmol, 1.0 equiv) in DMF (10 mL) at 4° C. was added NaH (137.92 mg, 3.45 mmol, 60 wt. %, 1.0 equiv). The mixture was stirred at 4° C. for 30 min and then a solution of tert-butyl (4-bromobutyl)(methyl)carbamate (1.01 g, 3.79 mmol, 25.92 mL, 1.1 equiv) in DMF (3 mL) was added. The mixture was stirred at room temperature for 3 h, at which point H₂O (100 mL) was added. The aqueous phase was extracted with EtOAc (3×30 mL) and the combined organic phases were washed with brine (20 mL), dried with anhydrous Na₂SO₄, filtered and concentrated under reduced pressure. The residue was purified by silica gel chromatography (1/0 to 0/1 petroleum ether/EtOAc) to afford tert-butyl (4-(4-amino-3-iodo-1H-pyrazolo[3,4-d]pyrimidin-1-yl)butyl) (methyl) carbamate (1.2 g, 78% yield) as a white solid. LCMS (ESI) m/z: [M+H] calcd for C₁₅H₂₃IN₆O₂: 447.10; found 447.1.

### Step 4: Synthesis of tert-butyl (4-(4-amino-3-(2-aminobenzo[d]oxazol-5-yl)-1H-pyrazolo[3,4-d]pyrimidin-1-yl)butyl)(methyl)carbamate

To a bi-phasic suspension of tert-butyl (4-(4-amino-3-iodo-1H-pyrazolo[3,4-d]pyrimidin-1-yl)butyl)(methyl)carbamate (1.2 g, 2.69 mmol, 1.0 equiv), 5-(4,4,5,5-tetramethyl-1,3,2-dioxaborolan-2-yl)benzo[d]oxazol-2-amine (1.19 g, 3.23 mmol, 1.2 equiv), and Na₂CO₃ (1.42 g, 13.44 mmol, 5.0 equiv) in DME (20 mL) and H₂O (10 mL) at room temperature was added Pd(PPh₃)₄ (310.71 mg, 268.89 μmol, 0.1 equiv) under N₂. The mixture was stirred at 110° C. for 3 h and then the reaction mixture was cooled and partitioned between EtOAc (20 mL) and H₂O (15 mL). The aqueous layer was separated and extracted with EtOAc (3×20 mL). The combined organic layers were washed with brine (2×20 mL), dried over anhydrous Na₂SO₄, filtered and concentrated under reduced pressure. The crude product was purified by silica gel chromatography (1/0 to 4/1 EtOAc/MeOH) to give tert-butyl (4-(4-amino-3-(2-aminobenzo[d]oxazol-5-yl)-1H-pyrazolo [3,4-d]pyrimidin-1-yl)butyl)(methyl) carbamate (0.78 g, 62.5% yield) as an orange solid.

### Step 5: Synthesis of 5-(4-amino-1-(4-(methylamino)butyl)-1H-pyrazolo[3,4-d]pyrimidin-3-yl) benzo[d]oxazol-2-amine

A solution of tert-butyl(4-(4-amino-3-(2-aminobenzo[d]oxazol-5-yl)-1H-pyrazolo[3,4-d]pyrimidin-1-yl)butyl)(methyl)carbamate (0.78 g, 1.72 mmol, 1.0 equiv) in TFA (5 mL) at room temperature was stirred for 30 min. The solution was concentrated under reduced pressure and the oily residue was triturated with MeCN (1 mL) and then added to MTBE (100 mL). The supernatant was removed and then the precipitate was collected by filtration under N₂ to give 5-(4-amino-1-(4-(methylamino) butyl)-1H-pyrazolo[3,4-d]pyrimidin-3-yl)benzo[d]oxazol-2-amine bis-trifluorosulfonate (0.959 g, 93% yield) as an orange solid. LCMS (ESI) m/z: [M+H] calcd for C₁₇H₂₀N₈O: 353.18; found 353.1.

### Monomer V. 1-(4-(4-(5-(aminomethyl)pyrimidin-2-yl)piperazin-1-yl)-3-(trifluoromethyl)phenyl)-8-(6-methoxypyridin-3-yl)-3-methyl-1,3-dihydro-2H-imidazo[4,5-c]quinolin-2-one

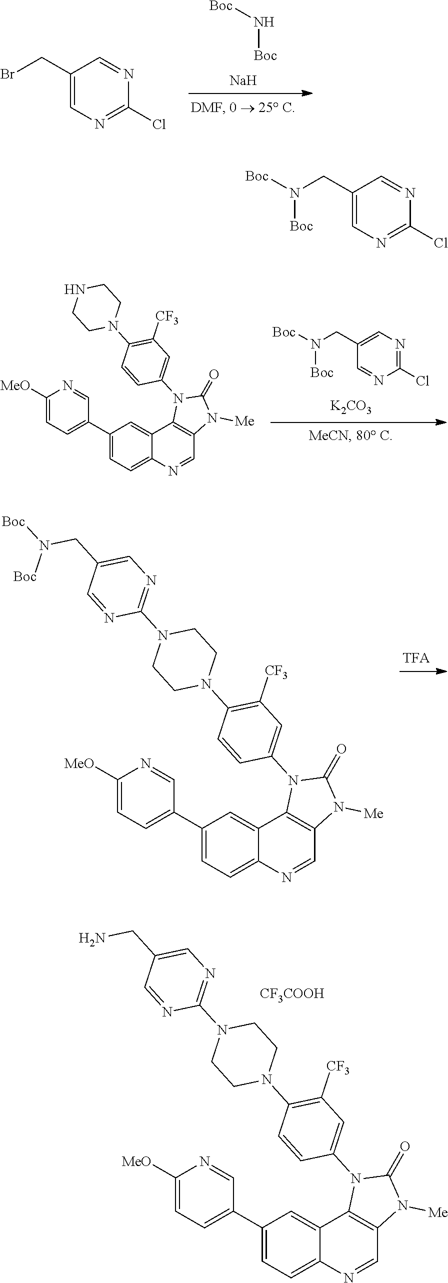

### Step 1: Synthesis of tert-butyl N-tert-butoxycarbonyl-N-[(2-chloropyrimidin-5-yl)methyl]carbamate

To a solution of tert-butyl N-tert-butoxycarbonylcarbamate (7.33 g, 33.74 mmol, 1.0 equiv) in DMF (80 mL) was added NaH (1.62 g, 40.49 mmol, 60 wt. %, 1.2 equiv) at 0° C. The mixture was stirred at 0° C. for 30 min and then 5-(bromomethyl)-2-chloro-pyrimidine (7 g, 33.74 mmol, 1 equiv) was added. The reaction mixture was stirred at room temperature for 1.5 h and then the mixture was poured into sat. NH₄Cl (300 mL) and stirred for 5 min. The aqueous phase was extracted with EtOAc (3×80 mL) and the combined organic phases were washed with brine (50 mL), dried over anhydrous Na₂SO₄, filtered and concentrated under reduced pressure. The residue was purified by silica gel chromatography (20:1 to 1:1 petroleum ether/EtOAc) to afford tert-butyl N-tert-butoxycarbonyl-N-[(2-chloro pyrimidin-5-yl)methyl]carbamate (7.0 g, 60.3% yield) as a white solid. LCMS (ESI) m/z: [M+H] calcd for C₁₅H₂₂ClN₃O₄: 344.14; found 344.2.

### Step 2: Synthesis of tert-butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate

To a solution of 8-(6-methoxy-3-pyridyl)-3-methyl-1-[4-piperazin-1-yl-3-(trifluoromethyl)phenyl]imidazo[4,5-c]quinolin-2-one (0.4 g, 748.32 μmol, 1.0 equiv) in MeCN (7 mL) was added tert-butyl N-tert-butoxycarbonyl-N-[(2-chloropyrimidin-5-yl)methyl]carbamate (514.55 mg, 1.50 mmol, 2.0 equiv) and K₂CO₃ (413.69 mg, 2.99 mmol, 4 equiv) at room temperature. The reaction mixture was stirred at 80° C. for 14 h and then the mixture was cooled to room temperature, filtered and concentrated under reduced pressure. The residue was purified by washing with MTBE (5 mL) to give tert-butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate (0.57 g, 90.5% yield) as a light yellow solid. LCMS (ESI) m/z: [M+H] calcd for C₄₃H₄₆F₃N₉O₆: 842.36; found 842.7.

### Step 3: Synthesis of 1-[4-[4-[5-(aminomethyl)pyrimidin-2-yl]piperazin-1-yl]-3-(trifluoromethyl) phenyl]-8-(6-methoxy-3-pyridyl)-3-methyl-imidazo[4,5-c]quinolin-2-one

A solution of tert-butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate (0.95 g, 1.13 mmol, 1 equiv) in TFA (10 mL) was stirred at room temperature for 1 h, at which point the solvent was concentrated. The residue was dissolved in MeCN (10 mL) and then the solution was added to MTBE (150 mL), dropwise. The precipitate was collected to give 1-[4-[4-[5-(aminomethyl)pyrimidin-2-yl]piperazin-1-yl]-3-(trifluoromethyl)phenyl]-8-(6-methoxy-3-pyridyl)-3-methyl-imidazo[4,5-c]quinolin-2-one trifluoromethanesulfonate (0.778 g, 84.8% yield) as a yellow solid. LCMS (ESI) m/z: [M+H] calcd for C₃₃H₃₀F₃N₉O₂: 642.26; found 642.4.

### Monomer W. 1-(4-aminobutyl)-3-(1H-pyrrolo[2,3-b]pyridin-5-yl)pyrazolo[3,4-d]pyrimidin-4-amine

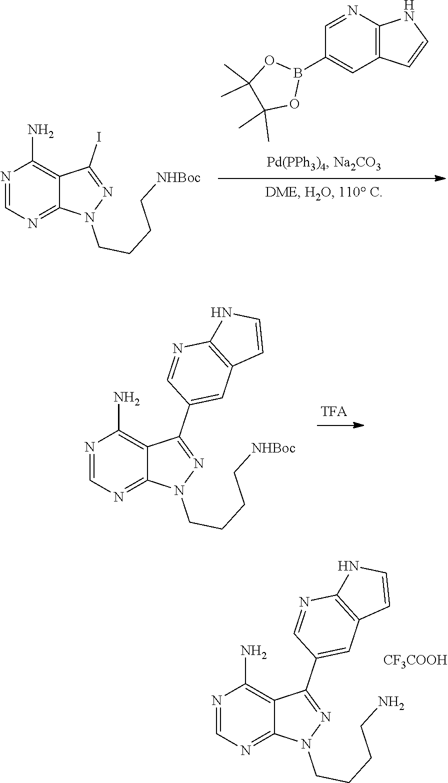

### Step 1: Synthesis of tert-butyl N-[4-[4-amino-3-(1H-indol-5-yl)pyrazolo[3,4-d]pyrimidin-1-yl]butyl]carbamate

To a bi-phasic suspension of tert-butyl N-[4-(4-amino-3-iodo-pyrazolo[3,4-d]pyrimidin-1-yl)butyl]carbamate (8 g, 18.51 mmol, 1 equiv), 5-(4,4,5,5-tetramethyl-1,3,2-dioxaborolan-2-yl)-1H-pyrrolo[2,3-b]pyridine (5.42 g, 22.21 mmol, 1.2 equiv) and Na₂CO₃ (9.81 g, 92.54 mmol, 5 equiv) in diglyme (160 mL) and H₂O (80 mL) was added Pd(PPh₃)₄ (2.14 g, 1.85 mmol, 0.1 equiv) at room temperature under N₂. The mixture was stirred at 110° C. for 3 h. The reaction mixture was cooled to room temperature, filtered and the filtrate was partitioned between EtOAc (500 mL) and H₂O (500 mL). The aqueous layer was separated and extracted with EtOAc (3×300 mL). The organic layers were combined, washed with brine (20 mL) and dried over anhydrous Na₂SO₄, then filtered and the filtrate was concentrated under reduced pressure. The residue was purified by silica gel chromatography (1/0 to 0/1 petroleum ether/EtOAc then 4/1 EtOAc/MeOH) to give tert-butyl N-[4-[4-amino-3-(1H-indol-5-yl)pyrazolo[3,4-d]pyrimidin-1-yl]butyl]carbamate (6.6 g, 84.6% yield) as a yellow solid. LCMS (ESI) m/z: [M+H] calcd for C₂₂H₂₇N₇O₂: 422.22; found 423.3.

### Step 2: Synthesis of 1-(4-aminobutyl)-3-(1H-pyrrolo[2,3-b]pyridin-5-yl)pyrazolo[3,4-d]pyrimidin-4-amine

To tert-butyl N-[4-[4-amino-3-(1H-indol-5-yl)pyrazolo[3,4-d]pyrimidin-1-yl]butyl]carbamate (6.6 g, 15.66 mmol, 1 equiv) was added TFA (66 mL), which was then stirred at room temperature for 30 min. The reaction solution was concentrated under reduced pressure to remove TFA and then MTBE (400 mL) was added to the residue. The suspension was stirred for 15 min, at which point the yellow solid was filtered, and the solid cake dried under reduced pressure to give 1-(4-aminobutyl)-3-(1H-pyrrolo[2,3-b]pyridin-5-yl)pyrazolo[3,4-d]pyrimidin-4-amine (10.2 g, 97.1% yield) as a yellow solid. LCMS (ESI) m/z: [M+H] calcd for C₁₆H₁₈N₈: 323.17; found 323.1.

### Monomer X. 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate

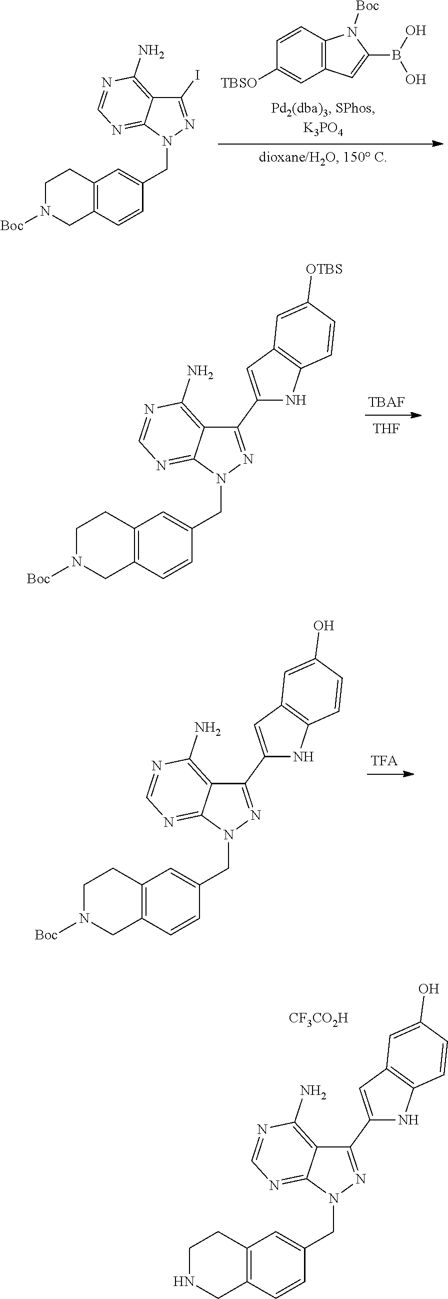

### Step 1: Synthesis of tert-butyl 6-((4-amino-3-(5-((tert-butyldimethylsilyl)oxy)-1H-indol-2-yl)-1H-pyrazolo[3,4-d]pyrimidin-1-yl)methyl)-3,4-dihydroisoquinoline-2(1H)-carboxylate

To a solution of tert-butyl 6-((4-amino-3-iodo-1H-pyrazolo[3,4-d]pyrimidin-1-yl)methyl)-3,4-dihydroisoquinoline-2(1H)-carboxylate (1 g, 1.97 mmol, 1.0 equiv) in dioxane (10.5 mL) and H₂O (3.5 mL) was added (1-(tert-butoxycarbonyl)-5-((tert-butyldimethylsilyl)oxy)-1H-indol-2-yl)boronic acid (1.16 g, 2.96 mmol, 1.5 equiv), K₃PO₄ (1.26 g, 5.92 mmol, 3.0 equiv), Pd₂(dba)₃ (180.85 mg, 197.50 μmol, 0.1 equiv), and SPhos (162.16 mg, 394.99 μmol, 0.2 equiv) at room temperature under N₂. The sealed tube was heated at 150° C. for 20 min under microwave. The reaction mixture was then cooled and 6 separate batches were combined together. The reaction mixture was partitioned between EtOAc (100 mL) and H₂O (100 mL). The aqueous layer was separated and extracted with EtOAc (3×80 mL). The organic layers were combined, washed with brine (100 mL) and dried over anhydrous Na₂SO₄. The solution was filtered and the filtrate was concentrated under reduced pressure. The crude material was purified by silica gel column chromatography (100/1 to 1/4 petroleum ether/EtOAc) to give tert-butyl 6-((4-amino-3-(5-((tert-butyldimethylsilyl)oxy)-1H-indol-2-yl)-1H-pyrazolo [3,4-d]pyrimidin-1-yl)methyl)-3,4-dihydroisoquinoline-2(1H)-carboxylate (6.17 g, 82.9% yield) as a light yellow solid.

### Step 2: Synthesis of tert-butyl 6-((4-amino-3-(5-hydroxy-1H-indol-2-yl)-1H-pyrazolo[3,4-d]pyrimidin-1-yl)methyl)-3,4-dihydroisoquinoline-2(1H)-carboxylate

To a mixture of tert-butyl 6-((4-amino-3-(5-((tert-butyldimethylsilyl)oxy)-1H-indol-2-yl)-1H-pyrazolo[3,4-d]pyrimidin-1-yl)methyl)-3,4-dihydroisoquinoline-2(1H)-carboxylate (6.17 g, 9.86 mmol, 1.0 equiv) in THF (100 mL) was added tetrabutylammonium fluoride trihydrate (1 M, 10.84 mL, 1.1 equiv) in one portion at 0° C. under N₂. The mixture was stirred at 0° C. for 1 h and was then added to H₂O (100 mL). The aqueous phase was extracted with EtOAc (3×80 mL) and the combined organic phase was washed with brine (2×80 mL), dried with anhydrous Na₂SO₄, filtered and concentrated under reduced pressure. The residue was purified by silica gel chromatography (1/1 to 0/1 petroleum ether/EtOAc) to afford tert-butyl 6-((4-amino-3-(5-hydroxy-1H-indol-2-yl)-1H-pyrazolo[3,4-d]pyrimidin-1-yl)methyl)-3,4-dihydroisoquinoline-2(1H)-carboxylate (4 g, 79.3% yield) as a light pink solid. LCMS (ESI) m/z: [M+H] calcd for C₂₈H₂₉N₇O₃: 512.24; found 512.3.

### Step 3: Synthesis of 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate

To a solution of tert-butyl 6-((4-amino-3-(5-hydroxy-1H-indol-2-yl)-1H-pyrazolo [3,4-d]pyrimidin-1-yl)methyl)-3,4-dihydroisoquinoline-2(1H)-carboxylate (4.5 g, 8.80 mmol, 1.0 equiv) in MeOH (50 mL) was added HCl in MeOH (4 M, 50 mL, 22.7 equiv) at room temperature. The mixture was stirred at room temperature overnight and was then concentrated under reduced pressure. To the crude product was added EtOAc (100 mL) and the resulting precipitate was collected by filtration under N₂ to give 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate (4.1 g, 85.0% yield, 3HCl) as a light yellow solid. LCMS (ESI) m/z: [M+H] calcd for C₂₃H₂₁N₇O: 412.19; found 412.1.

### Monomer Y. 3-(1H-pyrrolo[2,3-b]pyridin-5-yl)-1-((1,2,3,4-tetrahydroisoquinolin-6-yl)methyl)-1H-pyrazolo[3,4-d]pyrimidin-4-amine 2,2,2-trifluoroacetate

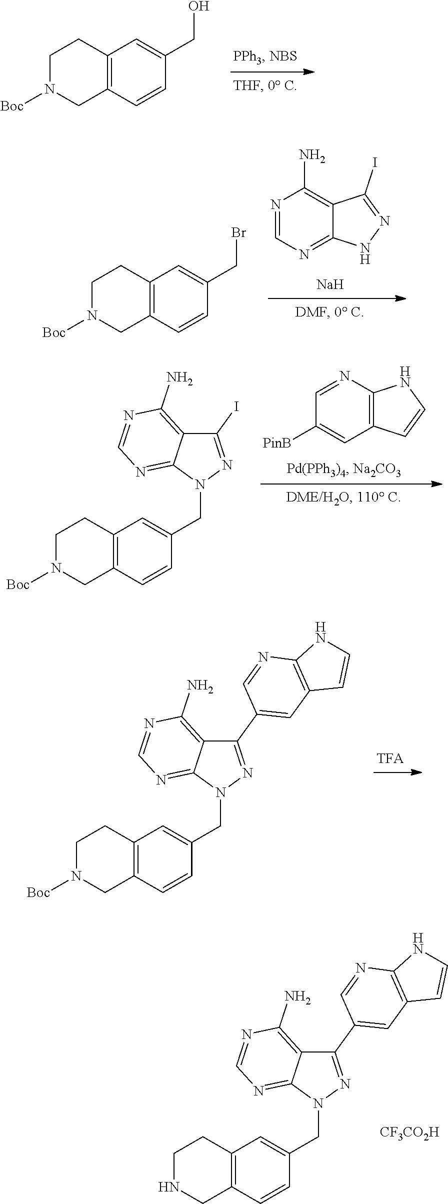

### Step 1: Synthesis of tert-butyl 6-(bromomethyl)-3,4-dihydroisoquinoline-2(1H)-carboxylate

A solution of NBS (34.07 g, 191.39 mmol, 4 equiv) in THF (200 mL) was added in portions to a solution of tert-butyl 6-(hydroxymethyl)-3,4-dihydroisoquinoline-2(1H)-carboxylate (12.6 g, 47.85 mmol, 1.0 equiv) and triphenylphosphine (37.65 g, 143.55 mmol, 3.0 equiv) in THF (200 mL) at 0° C. After the addition was complete, the mixture was stirred for 1 h at room temperature. EtOAc (150 mL) was added and the mixture was washed with H₂O (200 mL) and brine (150 mL), dried over anhydrous Na₂SO₄ and concentrated under reduced pressure. The residue was purified by silica gel chromatography (100/1 to 10/1 petroleum ether/EtOAc) to afford tert-butyl 6-(bromomethyl)-3,4-dihydroisoquinoline-2(1H)-carboxylate (8.56 g, 54.8% yield) as a light yellow solid.

### Step 2: Synthesis of tert-butyl 6-((4-amino-3-iodo-1H-pyrazolo[3,4-d]pyrimidin-1-yl) methyl)-3,4-dihydroisoquinoline-2(1H)-carboxylate

To a suspension of 3-iodo-1H-pyrazolo[3,4-d]pyrimidin-4-amine (9.5 g, 36.40 mmol, 1.0 equiv) in DMF (110 mL) was added NaH (1.46 g, 36.40 mmol, 60 wt. %, 1.0 equiv) at 0° C. The mixture was stirred at 0° C. for 30 min at which point a solution of tert-butyl 6-(bromomethyl)-3,4-dihydroisoquinoline-2(1H)-carboxylate (12.47 g, 38.22 mmol, 1.05 equiv) in DMF (40 mL) was added at 0° C. The mixture was stirred at room temperature for 1 h and then H₂O (1000 mL) was added at 0° C. The mixture stirred at 0° C. for 30 min and then the resulting precipitate was collected by filtration to give tert-butyl 6-((4-amino-3-iodo-1H-pyrazolo[3,4-d]pyrimidin-1-yl)methyl)-3,4-dihydroisoquinoline-2(1H)-carboxylate (17.8 g, 76.3% yield) as a light yellow solid, which was used the next step directly. LCMS (ESI) m/z: [M+H] calcd for C₂₀H₂₃IN₆O₂: 507.10; found 507.1.

### Step 3: Synthesis of tert-butyl 6-((4-amino-3-(1H-pyrrolo[2,3-b]pyridin-5-yl)-1H-pyrazolo[3,4-d]pyrimidin-1-yl)methyl)-3,4-dihydroisoquinoline-2(1H)-carboxylate

To a bi-phasic suspension of tert-butyl 6-((4-amino-3-iodo-1H-pyrazolo [3,4-d]pyrimidin-1-yl)methyl)-3,4-dihydroisoquinoline-2(1H)-carboxylate (6.5 g, 10.14 mmol, 1.0 equiv), 5-(4,4,5,5-tetramethyl-1,3,2-dioxaborolan-2-yl)-1H-pyrrolo [2,3-b]pyridine (2.97 g, 12.16 mmol, 1.2 equiv), and Na₂CO₃ (5.37 g, 50.68 mmol, 5.0 equiv) in diglyme (100 mL) and H₂O (50 mL) was added Pd(PPh₃)₄ (1.17 g, 1.01 mmol, 0.1 equiv) at room temperature under N₂. The mixture was stirred at 110° C. for 3 h. The reaction mixture was then cooled and partitioned between EtOAc (100 mL) and H₂O (100 mL). The aqueous layer was separated and extracted with EtOAc (2×100 mL). The combined organic phase was washed with brine (100 mL), dried with anhydrous Na₂SO₄, filtered and concentrated under reduced pressure. The residue was purified by silica gel chromatography (0/1 to 1/4 MeOH/EtOAc) to afford tert-butyl 6-((4-amino-3-(1H-pyrrolo[2,3-b]pyridin-5-yl)-1H-pyrazolo[3,4-d]pyramid in-1-yl) methyl)-3,4-dihydroisoquinoline-2(1H)-carboxylate (3.77 g, 72.1% yield) as a light yellow solid. LCMS (ESI) m/z: [M+H] calcd for C₂₇H₂₈N₈O₂: 497.24; found 497.3.

### Step 4: Synthesis of 3-(1H-pyrrolo[2,3-b]pyridin-5-yl)-1-((1,2,3,4-tetrahydroisoquinolin-6-yl)methyl)-1H-pyrazolo[3,4-d]pyrimidin-4-amine 2,2,2-trifluoroacetate

tert-Butyl 6-((4-amino-3-(1H-pyrrolo[2,3-b]pyridin-5-yl)-1H-pyrazolo[3,4-d]pyrimidin-1-yl)methyl)-3,4-dihydroisoquinoline-2(1H)-carboxylate (3.77 g, 7.59 mmol, 1.0 equiv) was added to TFA (85.36 mL, 1.15 mol, 151.8 equiv) at room temperature. The reaction mixture was stirred for 1 h. It was then concentrated under reduced pressure and the oily residue was triturated with MeCN (3 mL), then dripped into MTBE (200 mL) for 5 min. The supernatant was removed and then the precipitate was collected by filtration under N₂ to give the product, which was dissolved in MeCN (20 mL), and finally concentrated under reduced pressure to give 3-(1H-pyrrolo[2,3-b]pyridin-5-yl)-1-((1,2,3,4-tetrahydroisoquinolin-6-yl)methyl)-1H-pyrazolo[3,4-d]pyrimidin-4-amine 2,2,2-trifluoroacetate (4.84 g, 85.0% yield, 3TFA) as a light yellow solid. LCMS (ESI) m/z: [M+H] calcd for C₂₂H₂₀N₈: 397.19; found 397.2.

### Monomer Z. (4-((2-aminoethyl)sulfonyl)-3-fluoro-2-methylphenyl)(7-(6-aminopyridin-3-yl)-2,3-dihydrobenzo[f][1,4]oxazepin-4(5H)-yl)methanone 2,2,2-trifluoroacetate

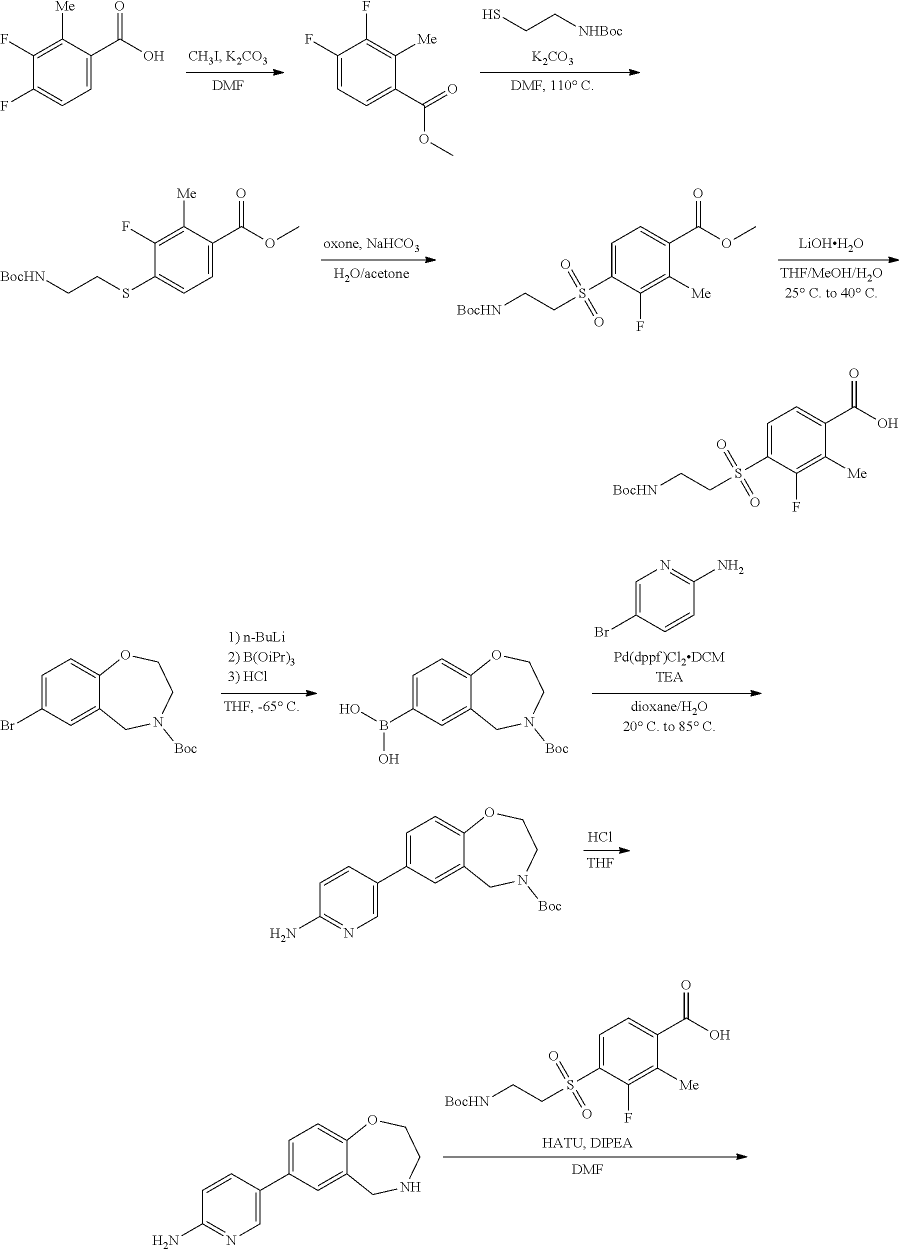

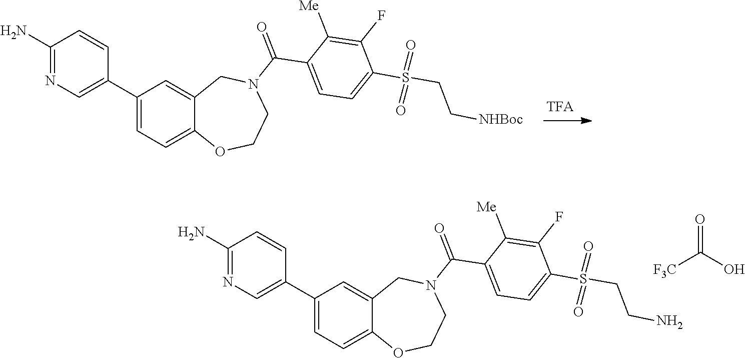 [SMILES: Cc1c(C(=O)N2CCOc3ccc(-c4ccc(N)nc4)cc3C2)ccc(S(=O)(=O)CCNC(=O)OC(C)(C)C)c1F ; Cc1c(C(=O)N2CCOc3ccc(-c4ccc(N)nc4)cc3C2)ccc(S(=O)(=O)CCN)c1F ; O=C(O)C(F)(F)F]

### Step 1: Synthesis of methyl 3,4-difluoro-2-methylbenzoate

To a solution of 3,4-difluoro-2-methylbenzoic acid (2 g, 11.62 mmol, 1.0 equiv) in DMF (20 mL) was added K₂CO₃ (4.82 g, 34.86 mmol, 3.0 equiv) and iodomethane (3.26 mL, 52.29 mmol, 4.5 equiv) at room temperature. The mixture was stirred at room temperature for 3 h. The solution of methyl 3,4-difluoro-2-methylbenzoate in DMF (20 mL) was used directly in the next step.

### Step 2: Synthesis of methyl 4-((2-((tert-butoxycarbonyl)amino)ethyl)thio)-3-fluoro-2-methylbenzoate

To a solution of methyl 3,4-difluoro-2-methylbenzoate (2.16 g, 11.28 mmol, 1.0 equiv) in DMF (20 mL) was added tert-butyl (2-mercaptoethyl)carbamate (2.0 g, 11.28 mmol, 1 equiv) and K₂CO₃ (3.12 g, 22.56 mmol, 2.0 equiv) at room temperature. The reaction was stirred at 110° C. for 12 h, at which point the mixture was added to H₂O (50 mL). The aqueous solution was then extracted with EtOAc (3×30 mL) and the organic phase was combined and concentrated under reduced pressure. The residue was purified by silica gel chromatography (1/0 to 3/1 petroleum ether/EtOAc) to afford methyl 4-((2-((tert-butoxycarbonyl)amino)ethyl)thio)-3-fluoro-2-methylbenzoate (3.0 g, 76% yield) as light yellow solid.

### Step 3: Synthesis of methyl 4-((2-((tert-butoxycarbonyl)amino)ethyl)sulfonyl)-3-fluoro-2-methylbenzoate

To a solution of methyl 4-((2-((tert-butoxycarbonyl)amino)ethyl)thio)-3-fluoro-2-methylbenzoate (3.3 g, 9.61 mmol, 1.0 equiv), NaOH (2 M, 4.80 mL, 1.0 equiv), and NaHCO₃ (2.42 g, 28.83 mmol, 3.0 equiv) in acetone (30 mL) was added potassium peroxymonosulfate (12.35 g, 20.08 mmol, 2.1 equiv). The mixture was stirred for 12 h at room temperature and then the mixture was acidified to pH 5 by addition of 1N HCl. The aqueous layer was extracted with EtOAc (3×30 mL) and the combined organic phase was washed with brine (20 mL), dried with anhydrous Na₂SO₄, filtered and concentrated under reduced pressure. The residue was purified by silica gel chromatography (1/0 to 3/1 petroleum ether/EtOAc) to afford methyl 4-((2-((tert-butoxycarbonyl)amino)ethyl)sulfonyl)-3-fluoro-2-methylbenzoate (2.1 g, 58.2% yield) as a yellow solid. LCMS (ESI) m/z: [M−56+H] calcd for C₁₆H₂₂FNO₆S: 320.12; found 320.1.

### Step 4: Synthesis of 4-((2-((tert-butoxycarbonyl)amino)ethyl)sulfonyl)-3-fluoro-2-methylbenzoic acid

To a solution of methyl 4-((2-((tert-butoxycarbonyl)amino)ethyl)sulfonyl)-3-fluoro-2-methylbenzoate (2.1 g, 5.59 mmol, 1.0 equiv) in THF (20 mL), MeOH (10 mL) and H₂O (10 mL) was added LiOH·H₂O (704.16 mg, 16.78 mmol, 3.0 equiv) at room temperature. The reaction mixture was stirred at 40° C. for 4 h. The mixture was then concentrated under reduced pressure to remove THF and MeOH. The aqueous phase was neutralized with 0.5N HCl and was then extracted with EtOAc (5×20 mL). The combined organic phase was washed with brine (2×20 mL), dried with anhydrous Na₂SO₄, filtered and concentrated under reduced pressure to give 4-((2-((tert-butoxycarbonyl)amino)ethyl)sulfonyl)-3-fluoro-2-methylbenzoic acid (2.01 g, 97.1% yield) as a white solid. LCMS (ESI) m/z: [M−100+H] calcd for C₁₅H₂₀FNO₆S: 262.11; found 262.1.

### Step 5: Synthesis of (4-(tert-butoxycarbonyl)-2,3,4,5-tetrahydrobenzo[f][1,4]oxazepin-7-yl)boronic acid

To a solution of tert-butyl 7-bromo-2,3-dihydrobenzo[f][1,4]oxazepine-4(5H)-carboxylate (4 g, 12.19 mmol, 1.0 equiv) in THF (80 mL) at −60° C. was added B(OiPr)₃ (4.58 g, 24.38 mmol, 5.60 mL, 2.0 equiv) followed by dropwise addition of n-BuLi (2.5 M, 12.19 mL, 2.5 equiv) in n-hexane. The reaction was stirred at −65° C. for 1 h. The reaction mixture was quenched with 1N HCl (12.25 mL) and allowed to warm to room temperature. The reaction mixture was extracted with EtOAc (3×30 mL), dried over anhydrous Na₂SO₄, filtered and concentrated under reduced pressure to give (4-(tert-butoxycarbonyl)-2,3,4,5-tetrahydrobenzo[f][1,4]oxazepin-7-yl)boronic acid (3.5 g, crude) as light yellow oil, which was used to the next step directly. LCMS (ESI) m/z: [M−100+H] calcd for C₁₄H₂₀BNO₅: 194.15; found 194.2.

### Step 6: Synthesis of tert-butyl 7-(6-aminopyridin-3-yl)-2,3-dihydrobenzo[f][1,4]oxazepine-4(5H)-carboxylate

To a solution of (4-(tert-butoxycarbonyl)-2,3,4,5-tetrahydrobenzo[f][1,4]oxazepin-7-yl)boronic acid (4.2 g, 14.33 mmol, 1.0 equiv) in H₂O (20 mL) and dioxane (60 mL) was added 5-bromopyridin-2-amine (2.48 g, 14.33 mmol, 1.0 equiv), Pd(dppf)Cl₂·DCM (1.17 g, 1.43 mmol, 0.1 equiv) and Et₃N (4.35 g, 42.99 mmol, 5.98 mL, 3.0 equiv) at room temperature. The mixture was stirred at 85° C. for 12 h. The mixture was then cooled to room temperature and the residue was poured into H₂O (15 mL). The aqueous phase was extracted with EtOAc (3×40 mL) and the combined organic phase was washed with brine (2×40 mL), dried with anhydrous Na₂SO₄, filtered and concentrated under reduced pressure. The residue was purified by silica gel chromatography (1/0 to 1/8 petroleum ether/EtOAc) to afford tert-butyl 7-(6-aminopyridin-3-yl)-2,3-dihydrobenzo[f][1,4]oxazepine-4(5H)-carboxylate (3.3 g, 65.0% yield) as light yellow solid. LCMS (ESI) m/z: [M+H] calcd for C₁₉H₂₃N₃O₃: 342.18; found 342.2.

### Step 7: Synthesis of 5-(2,3,4,5-tetrahydrobenzo[f][1,4]oxazepin-7-yl)pyridin-2-amine

To a solution of tert-butyl 7-(6-aminopyridin-3-yl)-2,3-dihydrobenzo[f][1,4]oxazepine-4(5H)-carboxylate (3.3 g, 9.67 mmol, 1.0 equiv) in THF (40 mL) was added HCl in EtOAc (4 M, 100 mL, 41.38 equiv) at room temperature. The mixture was stirred for 3 h. The reaction mixture was filtered and the filter cake was washed with EtOAc (3×15 mL) and then dried under reduced pressure to give 5-(2,3,4,5-tetrahydrobenzo [f][1,4]oxazepin-7-yl)pyridin-2-amine (3 g, 95.1% yield, 2HCl) as a light yellow solid.

### Step 8: Synthesis of tert-butyl (2-((4-(7-(6-aminopyridin-3-yl)-2,3,4,5-tetrahydrobenzo[f][1,4]oxazepine-4-carbonyl)-2-fluoro-3-methylphenyl)sulfonyl)ethyl)carbamate

To a solution of 4-((2-((tert-butoxycarbonyl)amino)ethyl)sulfonyl)-3-fluoro-2-methylbenzoic acid (690.08 mg, 1.91 mmol, 1.0 equiv) in DMF (10 mL) was added HATU (1.09 g, 2.86 mmol, 1.5 equiv) and DIPEA (1.66 mL, 9.55 mmol, 5 equiv). The reaction was stirred at room temperature for 30 min and then 5-(2,3,4,5-tetrahydrobenzo[f][1,4]oxazepin-7-yl)pyridin-2-amine (0.6 g, 1.91 mmol, 1.0 equiv, 2HCl) was added. The mixture was stirred for 2 h, at which point H₂O (40 mL) was added. The mixture was stirred for 5 min and the resulting precipitate was collected by filtration to give the crude product. The residue was purified by silica gel chromatography (1/0 to 10/1 EtOAc/MeOH) to afford tert-butyl (2-((4-(7-(6-aminopyridin-3-yl)-2,3,4,5-tetrahydrobenzo[f][1,4]oxazepine-4-carbonyl)-2-fluoro-3-methylphenyl)sulfonyl)ethyl)carbamate (0.538 g, 47.4% yield) as a light yellow solid. LCMS (ESI) m/z: [M+H] calcd for C₂₉H₃₃FN₄O₆S: 585.22; found 585.3.

### Step 9: Synthesis of (4-((2-aminoethyl)sulfonyl)-3-fluoro-2-methylphenyl)(7-(6-aminopyridin-3-yl)-2,3-dihydrobenzo[f][1,4]oxazepin-4(5H)-yl)methanone 2,2,2-trifluoroacetate

A solution tert-butyl (2-((4-(7-(6-aminopyridin-3-yl)-2,3,4,5-tetrahydrobenzo[f][1,4]oxazepine-4-carbonyl)-2-fluoro-3-methylphenyl)sulfonyl)ethyl)carbamate (0.538 g, 920.20 mol, 1.0 equiv) in TFA (10.35 mL, 139.74 mmol, 151.85 equiv) was stirred at room temperature for 2 h. The solution was then concentrated under reduced pressure. The oily residue was triturated with MeCN (1 mL) and then dripped into MTBE (30 mL) for 10 min. The supernatant was removed and then the precipitate was collected by filtration under N₂ to give (4-((2-aminoethyl)sulfonyl)-3-fluoro-2-methylphenyl)(7-(6-aminopyridin-3-yl)-2,3-dihydrobenzo[f][1,4]oxazepin-4(5H)-yl)methanone 2,2,2-trifluoroacetate (0.50 g, 87.4% yield) as light brown solid. LCMS (ESI) m/z: [M+H] calcd for C₂₄H₂₅FN₄O₄S: 485.17; found 485.1.

### Monomer AA. 5-(4-amino-1-(6-(piperazin-1-yl)pyrimidin-4-yl)-1H-pyrazolo[3,4-d]pyrimidin-3-yl)benzo[d]oxazol-2-amine trifluoroacetic acid salt

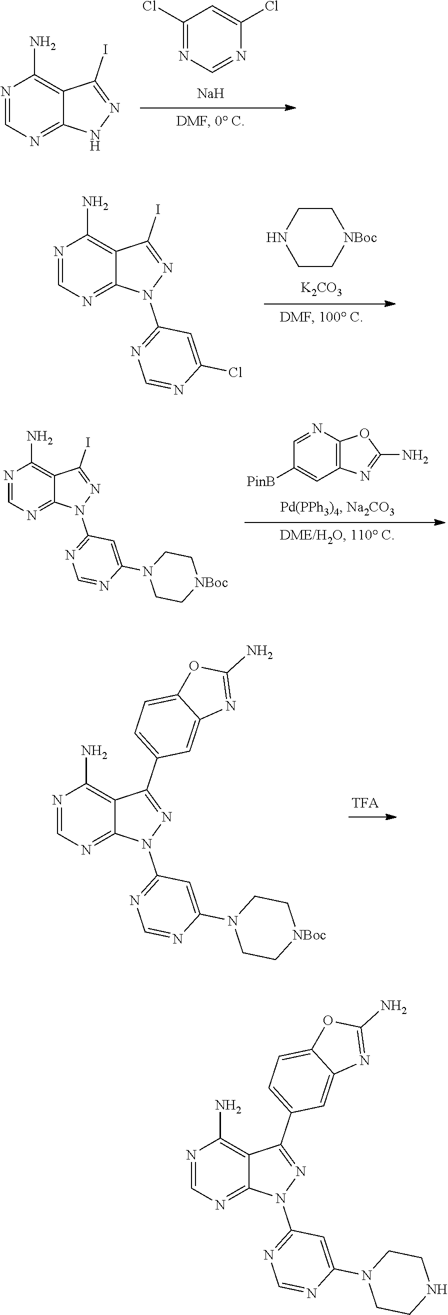

### Step 1: Synthesis of 1-(6-chloropyrimidin-4-yl)-3-iodo-1H-pyrazolo[3,4-d]pyrimidin-4-amine

To a suspension of 3-iodo-1H-pyrazolo[3,4-d]pyrimidin-4-amine (5 g, 19.16 mmol, 1.0 equiv) in DMF (60 mL) was added NaH (804.53 mg, 20.11 mmol, 60 wt. %, 1.05 equiv) at 0° C. The mixture was stirred at 0° C. for 30 min. To the reaction mixture was then added 4,6-dichloropyrimidine (3.42 g, 22.99 mmol, 1.2 equiv) at 0° C. The mixture was stirred at room temperature for 2.5 h, at which point the reaction mixture was added to H₂O (600 mL). The suspension was then filtered to give the product (7.1 g, 99.2% yield) as yellow solid. LCMS (ESI) m/z: [M+H] calcd for C₉H₅ClIN₇: 373.94; found 373.9.

### Step 2: Synthesis of tert-butyl 4-(6-(4-amino-3-iodo-1H-pyrazolo[3,4-d]pyrimidin-1-yl)pyrimidin-4-yl)piperazine-1-carboxylate

To a solution of 1-(6-chloropyrimidin-4-yl)-3-iodo-1H-pyrazolo[3,4-d]pyrimidin-4-amine (5 g, 13.39 mmol, 1.0 equiv) and tert-butyl piperazine-1-carboxylate (2.99 g, 16.06 mmol, 1.2 equiv) in DMF (50 mL) was added K₂CO₃ (3.70 g, 26.77 mmol, 2.0 equiv). The reaction mixture was stirred at 100° C. for 4 h, at which point it was added to H₂O (500 mL). The suspension was then filtered to give the product (6.2 g, 88.5% yield) as yellow solid. LCMS (ESI) m/z: [M+H] calcd for C₁₈H₂₂IN₉O₂: 524.09; found 524.2.

### Step 3: Synthesis of tert-butyl 4-(6-(4-amino-3-(2-aminobenzo[d]oxazol-5-yl)-1H-pyrazolo[3,4-d]pyrimidin-1-yl)pyrimidin-4-yl)piperazine-1-carboxylate

To a bi-phasic suspension of 5-(4,4,5,5-tetramethyl-1,3,2-dioxaborolan-2-yl)benzo[d]oxazol-2-amine (3.08 g, 11.85 mmol, 1.0 equiv), tert-butyl 4-(6-(4-amino-3-iodo-1H-pyrazolo[3,4-d]pyrimidin-1-yl)pyrimidin-4-yl)piperazine-1-carboxylate (6.2 g, 11.85 mmol, 1.0 equiv) and Na₂CO₃ (6.28 g, 59.24 mmol, 5.0 equiv) in H₂O (100 mL) and DME (200 mL) was added Pd(PPh₃)₄ (1.37 g, 1.18 mmol, 0.1 equiv) at room temperature under N₂. The mixture was stirred at 110° C. for 24 h and then the mixture was filtered to give a solid cake. The solid was added to dioxane (20 mL) and stirred at 110° C. for 60 min, then filtered to give the product (3.5 g, 55.8% yield) as brown solid. LCMS (ESI) m/z: [M+H] calcd for C₂₅H₂₇N₁₁O₃: 530.24; found 530.3.

### Step 4: Synthesis of 5-(4-amino-1-(6-(piperazin-1-yl)pyrimidin-4-yl)-1H-pyrazolo[3,4-d]pyrimidin-3-yl)benzo[d]oxazol-2-amine trifluoroacetic acid salt

A solution of tert-butyl 4-(6-(4-amino-3-(2-aminobenzo[d]oxazol-5-yl)-1H-pyrazolo[3,4-d]pyrimidin-1-yl)pyrimidin-4-yl)piperazine-1-carboxylate (3.5 g, 6.61 mmol, 1.0 equiv) in TFA (35 mL) was stirred at room temperature for 1 h. The reaction solution was concentrated under reduced pressure and the resulting crude material was dissolved in MeCN (20 mL) and added dropwise to MTBE (500 mL). The resulting solid was then filtered to give the product (5.5 g, 91.9% yield) as brown solid. LCMS (ESI) m/z: [M+H] calcd for C₂₀H₁₉N₁₁O: 430.19; found 430.1.

### Monomer AB. 8-(6-methoxypyridin-3-yl)-3-methyl-1-(4-(4-(5,6,7,8-tetrahydropyrido[4,3-d]pyrimidin-2-yl)piperazin-1-yl)-3-(trifluoromethyl)phenyl)-1H-imidazo[4,5-c]quinolin-2(3H)-one trifluoroacetic acid salt

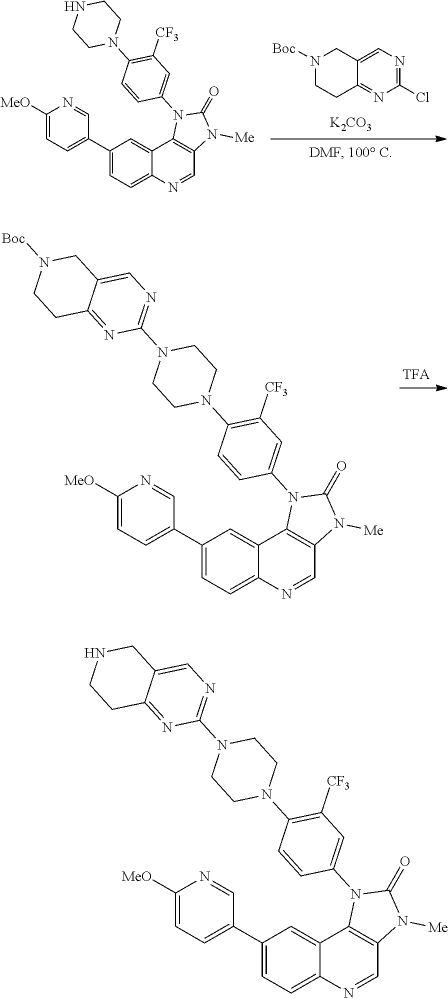

### Step 1: Synthesis of tert-butyl 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)-7,8-dihydropyrido[4,3-d]pyrimidine-6(5H)-carboxylate

To a mixture of 8-(6-methoxypyridin-3-yl)-3-methyl-1-(4-(piperazin-1-yl)-3-(trifluoromethyl)phenyl)-1H-imidazo[4,5-c]quinolin-2(3H)-one (0.3 g, 561.24 μmol, 1.0 equiv) and tert-butyl 2-chloro-7,8-dihydropyrido[4,3-d]pyrimidine-6(5H)-carboxylate (151.38 mg, 561.24 μmol, 1.0 equiv) in DMF (5 mL) was added K₂CO₃ (193.92 mg, 1.40 mmol, 2.5 equiv). The mixture was stirred at 100° C. for 14 h, at which point H₂O (20 mL) was added. The aqueous layer was extracted with EtOAc (3×40 mL) and the combined organic layers were concentrated under reduced pressure. The crude material was purified by column chromatography (30/1 to 15/1 DCM/MeOH) to give the product (0.30 g, 69.6% yield) as a light-yellow solid. LCMS (ESI) m/z: [M+H] calcd for C₄₀H₄₀F₃N₉O₄: 768.33; found 768.5.

### Step 2: Synthesis of 8-(6-methoxypyridin-3-yl)-3-methyl-1-(4-(4-(5,6,7,8-tetrahydropyrido[4,3-d]pyrimidin-2-yl)piperazin-1-yl)-3-(trifluoromethyl)phenyl)-1H-imidazo[4,5-c]quinolin-2(3H)-one

A solution of tert-butyl 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)-7,8-dihydropyrido[4,3-d]pyrimidine-6(5H)-carboxylate (0.8 g, 1.04 mmol, 1.0 equiv) in TFA (8 mL) was stirred at room temperature for 2 h. The solvent was concentrated and the residue was dissolved in MeCN (5 mL), then the solution was added dropwise to MTBE (150 mL). The precipitate was filtered and the solid was dried under reduced pressure to give the product (600 mg, 70.6% yield) as a yellow solid. LCMS (ESI) m/z: [M+H] calcd for C₃₅H₃₂F₃N₉O₂: 668.27; found 668.3.

### Monomer AC. 5-(4-amino-1-(piperidin-4-ylmethyl)-1H-pyrazolo[3,4-d]pyrimidin-3-yl)benzo[d]oxazol-2-amine trifluoroacetic acid salt

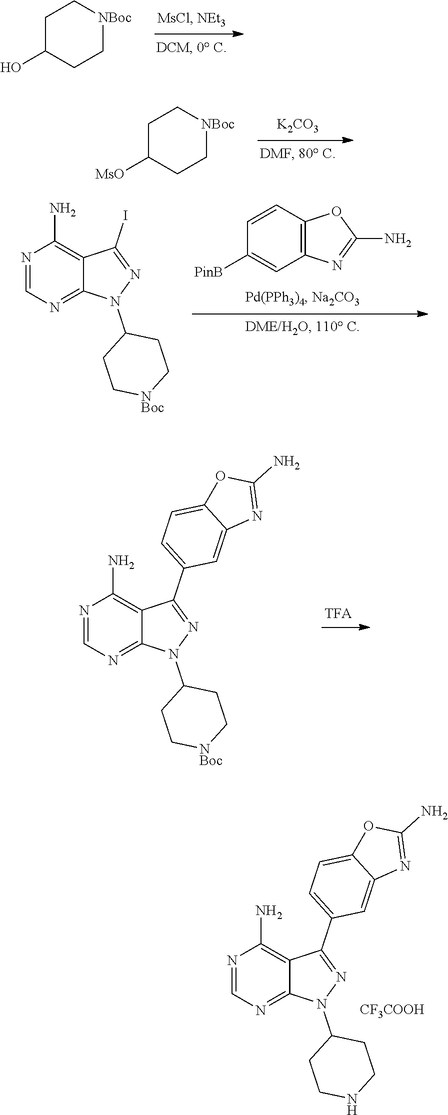

### Step 1: Synthesis of tert-butyl 4-((methylsulfonyl)oxy)piperidine-1-carboxylate

To a solution of tert-butyl 4-hydroxypiperidine-1-carboxylate (4 g, 19.87 mmol, 1.0 equiv) and Et₃N (3.87 mL, 27.82 mmol, 1.4 equiv) in DCM (40 mL) was added MsCl (2.15 mL, 27.82 mmol, 1.4 equiv) at 0° C. Then the reaction mixture was stirred at room temperature for 1 h. H₂O (50 mL) was added and the aqueous phase was extracted with DCM (3×50 mL). The combined organic phase was washed with brine, dried with anhydrous Na₂SO₄, filtered and concentrated under reduced pressure to give the product (5.62 g, 101% crude yield) as yellow solid which was used directly in the next step.

### Step 2: Synthesis of tert-butyl 4-(4-amino-3-iodo-1H-pyrazolo[3,4-d]pyrimidin-1-yl)piperidine-1-carboxylate

To a suspension of 3-iodo-1H-pyrazolo[3,4-d]pyrimidin-4-amine (5 g, 19.16 mmol, 1.0 equiv) and tert-butyl 4-((methylsulfonyl)oxy)piperidine-1-carboxylate (5.62 g, 20.11 mmol, 1.05 equiv) in DMF (100 mL) was added K₂CO₃ (5.29 g, 38.31 mmol, 2.0 equiv). The mixture was stirred at 80° C. for 12 h. The reaction mixture was then added to H₂O (400 mL) at 0° C. The resulting precipitate was filtered to give the product (5.0 g, 58.8% yield) as yellow solid. LCMS (ESI) m/z: [M+H] calcd for C₁₅H₂₁IN₆O₂: 445.09; found 445.1.

### Step 3: Synthesis of tert-butyl 4-(4-amino-3-(2-aminobenzo[d]oxazol-5-yl)-1H-pyrazolo[3,4-d]pyrimidin-1-yl)piperidine-1-carboxylate

To a suspension of tert-butyl 4-(4-amino-3-iodo-1H-pyrazolo[3,4-d]pyrimidin-1-yl)piperidine-1-carboxylate (5 g, 11.25 mmol, 1.0 equiv), 5-(4,4,5,5-tetramethyl-1,3,2-dioxaborolan-2-yl)benzo[d]oxazol-2-amine (3.51 g, 13.51 mmol, 1.2 equiv) and Na₂CO₃ (5.96 g, 56.27 mmol, 5.0 equiv) in H₂O (50 mL) and DME (100 mL) was added Pd(PPh₃)₄ (1.30 g, 1.13 mmol, 0.1 equiv) at room temperature under N₂. The mixture was stirred at 110° C. for 3 h. The reaction mixture was then cooled to room temperature and filtered. The filtrate was partitioned between EtOAc (100 mL) and H₂O (100 mL) and then the aqueous layer was separated and extracted with EtOAc (3×100 mL). The combined organic layer was washed with brine (20 mL) and dried over anhydrous Na₂SO₄, filtered, and concentrated under reduced pressure. The residue was triturated with EtOAc (30 mL) and filtered to give the product (3.6 g, 71% yield) as yellow solid. LCMS (ESI) m/z: [M+H] calcd for C₂₂H₂₆N₈O₃: 451.22; found 451.3.

### Step 4: Synthesis of 5-(4-amino-1-(piperidin-4-yl)-1H-pyrazolo[3,4-d]pyrimidin-3-yl)benzo[d]oxazol-2-amine trifluoroacetic acid salt

A solution of tert-butyl 4-(4-amino-3-(2-aminobenzo[d]oxazol-5-yl)-1H-pyrazolo[3,4-d]pyrimidin-1-yl)piperidine-1-carboxylate (1.4 g, 3.11 mmol, 1.0 equiv) in TFA (10 mL) was stirred at room temperature for 30 min. The reaction solution was concentrated under reduced pressure and the crude solid was dissolved in MeCN (20 mL). The solution was added dropwise to MTBE (100 mL) and the resulting solid was filtered to give the product (1.6 g, 85.8% yield) as yellow solid. LCMS (ESI) m/z: [M+H] calcd for C₁₇H₁₈N₈O₃: 351.17; found 351.1.

### Monomer AD. 1-(piperidin-4-yl)-3-(1H-pyrrolo[2,3-b]pyridin-5-yl)-1H-pyrazolo[3,4-d]pyrimidin-4-amine trifluoroacetic acid salt

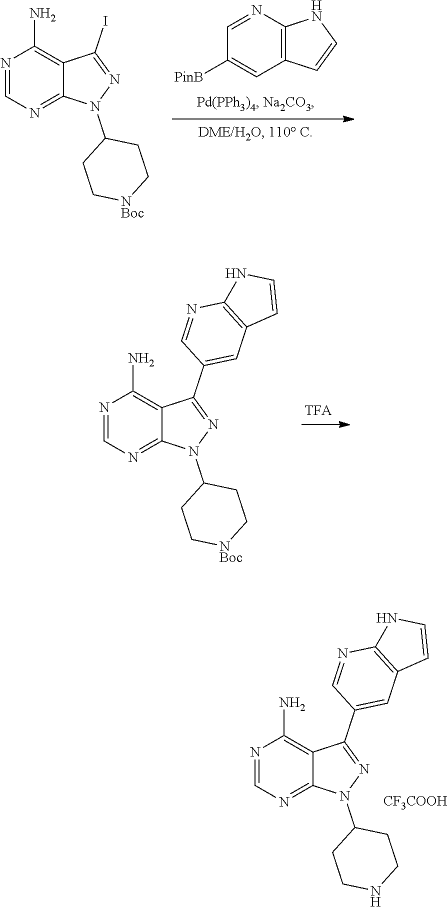

### Step 1: Synthesis of tert-butyl 4-(4-amino-3-(1H-pyrrolo[2,3-b]pyridin-5-yl)-1H-pyrazolo[3,4-d]pyrimidin-1-yl)piperidine-1-carboxylate

To a suspension of 5-(4,4,5-trimethyl-1,3,2-dioxaborolan-2-yl)-1H-pyrrolo[2,3-b]pyridine (857.12 mg, 3.51 mmol, 1.2 equiv), tert-butyl 4-(4-amino-3-iodo-1H-pyrazolo[3,4-d]pyrimidin-1-yl)piperidine-1-carboxylate (1.3 g, 2.93 mmol, 1.0 equiv) and Na₂CO₃ (1.55 g, 14.63 mmol, 5.0 equiv) in DME (20 mL) and H₂O (10 mL) was added Pd(PPh₃)₄ (338.13 mg, 292.62 μmol, 0.1 equiv) at room temperature under N₂. The mixture was stirred at 110° C. for 3 h. The reaction mixture was then cooled to room temperature and filtered. The filtrate was partitioned between EtOAc (50 mL) and H₂O (50 mL) and the aqueous layer was separated and extracted with EtOAc (3×50 mL). The combined organic layer were washed with brine, dried over anhydrous Na₂SO₄, filtered, and concentrated under reduced pressure. The residue was triturated with EtOAc (10 mL), filtered, the solid cake was dried under reduced pressure to give the product (1.0 g, 78.7% yield) as yellow solid.

### Step 2: Synthesis of 1-(piperidin-4-yl)-3-(1H-pyrrolo[2,3-b]pyridin-5-yl)-1H-pyrazolo[3,4-d]pyrimidin-4-amine trifluoroacetic acid salt

A solution of tert-butyl 4-(4-amino-3-(1H-pyrrolo[2,3-b]pyridin-5-yl)-1H-pyrazolo[3,4-d]pyrimidin-1-yl)piperidine-1-carboxylate (1.5 g, 3.45 mmol, 1.0 equiv) in TFA (10 mL) was stirred at room temperature for 30 min. The reaction solution was concentrated under reduced pressure and the crude residue was dissolved in MeCN (20 mL). The solution was added dropwise to MTBE (100 mL) and the resulting solid was filtered to give the product (1.19 g, 74.2% yield) as light yellow solid. LCMS (ESI) m/z: [M+H] calcd for C₁₇H₁₈N₈: 335.18; found 335.1.

### Monomer AE. (4-((2-aminoethyl)sulfonyl)-2-methylphenyl)(7-(6-aminopyridin-3-yl)-2,3-dihydrobenzo[f][1,4]oxazepin-4(5H)-yl)methanone

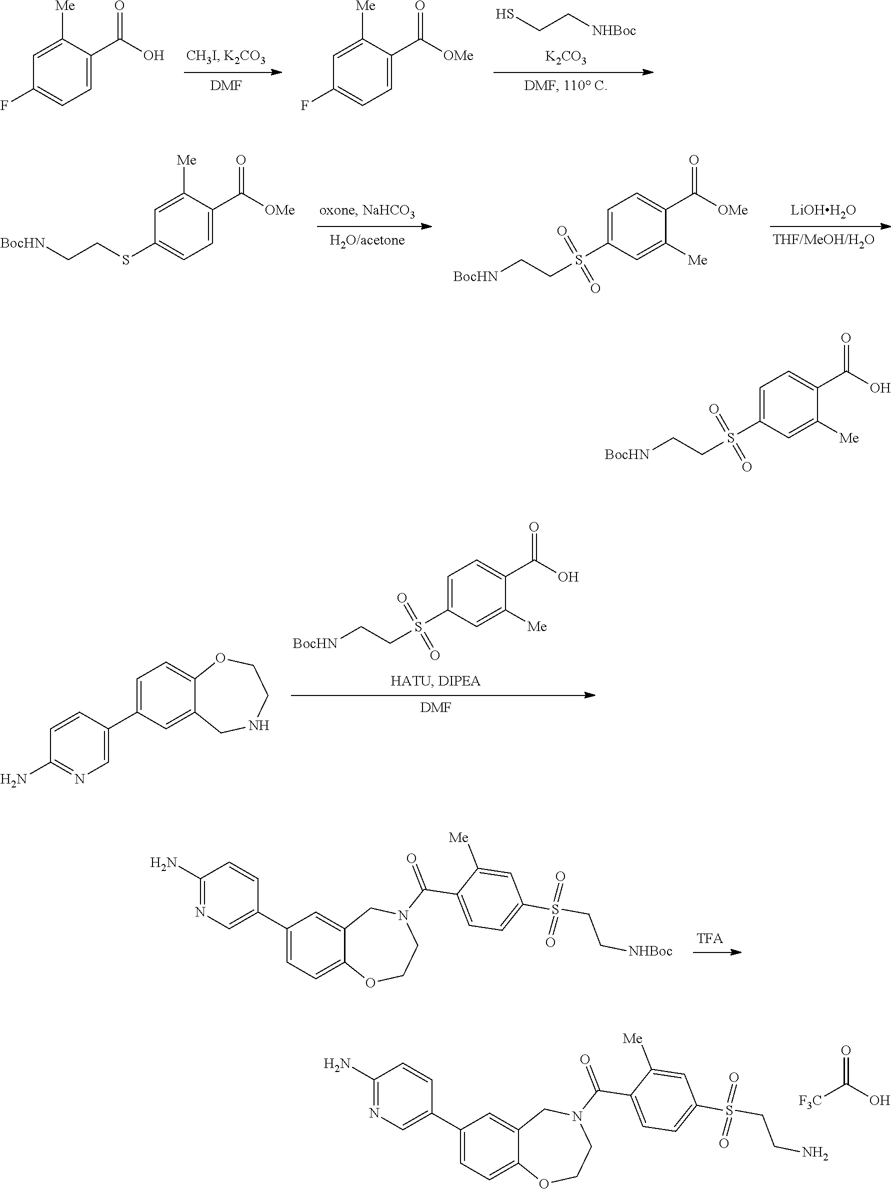

### Step 1: Synthesis of methyl 4-fluoro-2-methylbenzoate

To a solution of 4-fluoro-2-methylbenzoic acid (86 g, 557.94 mmol, 1.0 equiv) in DMF (900 mL) was added K₂CO₃ (231.33 g, 1.67 mol, 3.0 equiv) and iodomethane (79.19 g, 557.94 mmol, 34.73 mL, 1.0 equiv). The mixture was stirred at room temperature for 1 h. The solution of methyl 4-fluoro-2-methylbenzoate in DMF (900 mL) was used directly in the next step.

### Step 2: Synthesis of methyl 4-((2-((tert-butoxycarbonyl)amino)ethyl)thio)-2-methylbenzoate

To a solution of methyl 4-fluoro-2-methylbenzoate (93.8 g, 557.94 mmol, 1.0 equiv) in DMF (900 mL) was added tert-butyl (2-mercaptoethyl)carbamate (98.91 g, 557.97 mmol, 1.0 equiv) and K₂CO₃ (154.23 g, 1.12 mol, 2.0 equiv). The reaction was stirred at 110° C. for 12 h, at which point the mixture was cooled to room temperature and added to H₂O (1000 mL). The aqueous layer was then extracted with EtOAc (3×600 mL) and the combined organic layers were washed with brine, dried, and concentrated under reduced pressure. Purification by silica gel chromatography (0→25% EtOAc/petroleum ether) afforded the desired product as a colorless oil (144 g, 79% yield).

### Step 3: Synthesis of methyl 4-((2-((tert-butoxycarbonyl)amino)ethyl)sulfonyl)-2-methylbenzoate

To two separate batches containing a solution of methyl 4-((2-((tert-butoxycarbonyl)amino)ethyl)thio)-2-methylbenzoate (72 g, 221.25 mmol, 1.0 equiv), NaOH (2 M, 110.6 mL, 1.0 equiv), and NaHCO₃ (55.76 g, 663.75 mmol, 3.0 equiv) in acetone (750 mL) was added potassium peroxymonosulfate (284.28 g, 462.41 mmol, 2.1 equiv). The mixture was stirred for 12 h at room temperature, at which point the two batches were combined and then the mixture was acidified to pH 5 by addition of 1N HCl. The aqueous layer was extracted with EtOAc (3×1500 mL) and the combined organic phases were washed with brine (2×500 mL), dried, and concentrated under reduced pressure. Purification by silica gel chromatography (0→25% EtOAc/petroleum ether) afforded the desired product as a white solid (120 g, 76% yield).

### Step 4: Synthesis of 4-((2-((tert-butoxycarbonyl)amino)ethyl)sulfonyl)-2-methylbenzoic acid

To a solution of methyl 4-((2-((tert-butoxycarbonyl)amino)ethyl)sulfonyl)-2-methylbenzoate (35 g, 97.92 mmol, 1.0 equiv) in THF (200 mL), MeOH (100 mL) and H₂O (100 mL) was added LiOH·H₂O (12.33 g, 293.77 mmol, 3.0 equiv) at room temperature. The reaction mixture was stirred at 40° C. for 1 h. The mixture was then concentrated under reduced pressure to remove THF and MeOH. The aqueous phase was neutralized with 0.5N HCl and the resulting precipitate was isolated by filtration. The solid cake was washed with H₂O (3×20 mL) to afford the desired product as a white solid (25 g, 74% yield).

### Step 5: Synthesis of tert-butyl (2-((4-(7-(6-aminopyridin-3-yl)-2,3,4,5-tetrahydrobenzo[f][1,4]oxazepine-4-carbonyl)-3-methylphenyl)sulfonyl)ethyl)carbamate

To a solution of 4-((2-((tert-butoxycarbonyl)amino)ethyl)sulfonyl)-2-methylbenzoic acid (9.7 g, 28.25 mmol, 1.0 equiv) and 5-(2,3,4,5-tetrahydrobenzo[f][1,4]oxazepin-7-yl)pyridin-2-amine (8.88 g, 28.25 mmol, 1.0 equiv, 2HCl) in DMF (120 mL) was added HATU (16.11 g, 42.37 mmol, 1.5 equiv) and DIPEA (18.25 g, 141.24 mmol, 24.60 mL, 5.0 equiv). The reaction was stirred at room temperature for 1 h, at which point the reaction mixture was poured into H₂O (1000 mL). The mixture was stirred for 5 min and the resulting precipitate was collected by filtration to give the crude product. The crude product was triturated with EtOAc (100 mL), filtered, and the solid cake was dried under reduced pressure to afford the desired product as a white solid (14 g, 87% yield).

### Step 6: Synthesis of (4-((2-aminoethyl)sulfonyl)-2-methylphenyl)(7-(6-aminopyridin-3-yl)-2,3-dihydrobenzo[f][1,4]oxazepin-4(5H)-yl)methanone

A solution tert-butyl (2-((4-(7-(6-aminopyridin-3-yl)-2,3,4,5-tetrahydrobenzo[f][1,4]oxazepine-4-carbonyl)-3-methylphenyl)sulfonyl)ethyl)carbamate (19 g, 33.53 mmol, 1.0 equiv) in TFA (100 mL) was stirred at room temperature for 30 min. The solution was then concentrated under reduced pressure. The residue was triturated with MeCN (30 mL) and then dripped into MTBE (600 mL) and stirred for 20 min. The suspension was filtered and the resulting solid was dissolved in MeCN (30 mL) and concentrated under reduced pressure to afford the desired product as a light yellow solid (24 g, TFA salt). LCMS (ESI) m/z: [M+H] calcd for C₂₄H₂₆N₄O₄S: 467.18; found 467.1.

### Monomer AF. 5-(4-amino-1-((5,6,7,8-tetrahydropyrido[4,3-d]pyrimidin-2-yl)methyl)-1H-pyrazolo[3,4-d]pyrimidin-3-yl)benzo[d]oxazol-2-amine

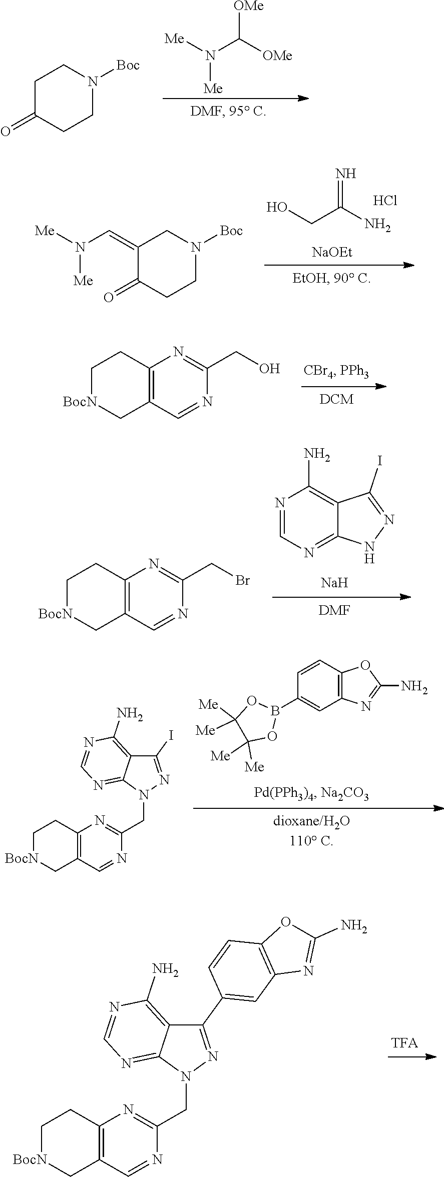

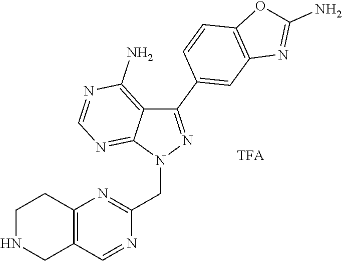 [SMILES: Nc1nc2cc(-c3nn(Cc4ncc5c(n4)CCNC5)c4ncnc(N)c34)ccc2o1]

### Step 1: Synthesis of (Z)-tert-butyl 3-((dimethylamino)methylene)-4-oxopiperidine-1-carboxylate

A solution of tert-butyl 4-oxopiperidine-1-carboxylate (15 g, 75.28 mmol, 1.0 equiv) and 1,1-dimethoxy-N,N-dimethylmethanamine (11.00 mL, 82.81 mmol, 1.1 equiv) in DMF (105 mL) was stirred at 95° C. for 12 h. The reaction mixture was then concentrated under reduced pressure and the resulting residue was dissolved in EtOAc (30 mL) and washed with brine (3×30 mL). The aqueous phase was extracted with EtOAc (50 mL), and the combined organic phases were dried and concentrated under reduced pressure to afford the desired product as a yellow solid (10.1 g, 53% yield).

### Step 2: Synthesis of tert-butyl 2-(hydroxymethyl)-7,8-dihydropyrido[4,3-d]pyrimidine-6(5H)-carboxylate

To a solution of NaOEt (1.98 g, 29.10 mmol, 1.0 equiv) in EtOH (70 mL) was added (Z)-tert-butyl 3-((dimethylamino)methylene)-4-oxopiperidine-1-carboxylate (7.4 g, 29.10 mmol, 1.0 equiv) and 2-hydroxyacetimidamide hydrochloride (3.54 g, 32.01 mmol, 1.1 equiv). The reaction mixture was heated to 90° C. for 12 h, at which point the mixture was cooled to room temperature and concentrated under reduced pressure. The residue was partitioned with EtOAc (40 mL) and washed with sat. NaHCO₃ (40 mL). The aqueous phase was extracted with EtOAc (3×20 mL) and the combined organic phases were washed with brine (2×50 mL), dried, and concentrated under reduced pressure. Purification by silica gel chromatography (25% EtOAc/petroleum ether) afforded the desired product as a yellow solid (7.24 g, 94% yield).

### Step 3: Synthesis of tert-butyl 2-(bromomethyl)-7,8-dihydropyrido[4,3-d]pyrimidine-6(5H)-carboxylate

To a solution of tert-butyl 2-(hydroxymethyl)-7,8-dihydropyrido[4,3-d]pyrimidine-6(5H)-carboxylate (6.24 g, 23.52 mmol, 1.0 equiv) and PPh₃ (12.34 g, 47.04 mmol, 2.0 equiv) in DCM (140 mL) was added CBr₄ (14.82 g, 44.69 mmol, 1.9 equiv). The mixture was stirred at room temperature for 3 h, at which point mixture was concentrated under reduced pressure. The residue was partitioned between EtOAc (20 mL) and H₂O (20 mL), the aqueous phase was extracted with EtOAc (3×20 mL). The combined organic phases were washed with brine (2×50 mL), dried, and concentrated under reduced pressure. Purification by silica gel chromatography (14% EtOAc/petroleum ether) afforded the desired product as a yellow solid (3.6 g, 47% yield).

### Step 4: Synthesis of tert-butyl 2-((4-amino-3-iodo-1H-pyrazolo[3,4-d]pyrimidin-1-yl)methyl)-7,8-dihydropyrido[4,3-d]pyrimidine-6(5H)-carboxylate

To a solution of 3-iodo-1H-pyrazolo[3,4-d]pyrimidin-4-amine (1.59 g, 6.09 mmol, 1.0 equiv) in DMF (15 mL) was added NaH (243.73 mg, 6.09 mmol, 60 wt. %, 1.0 equiv) at 0° C. The suspension was stirred for 30 min and then tert-butyl 2-(bromomethyl)-7,8-dihydropyrido[4,3-d]pyrimidine-6(5H)-carboxylate (2.2 g, 6.70 mmol, 1.1 equiv) was added. The reaction mixture was warmed to room temperature and stirred for 3 h. The mixture was poured into H₂O at 0° C. and the precipitate was collected by filtration to afford the desired product as a brown solid (2.5 g, 66% yield).

### Step 5: Synthesis of tert-butyl 2-((4-amino-3-(2-aminobenzo[d]oxazol-5-yl)-1H-pyrazolo[3,4-d]pyrimidin-1-yl)methyl)-7,8-dihydropyrido[4,3-d]pyrimidine-6(5H)-carboxylate

To a solution of tert-butyl 2-((4-amino-3-iodo-1H-pyrazolo[3,4-d]pyrimidin-1-yl)methyl)-7,8-dihydropyrido[4,3-d]pyrimidine-6(5H)-carboxylate (4.55 g, 8.95 mmol, 1.0 equiv), 5-(4,4,5,5-tetramethyl-1,3,2-dioxaborolan-2-yl)benzo[d]oxazol-2-amine (2.79 g, 10.74 mmol, 1.2 equiv) and Na₂CO₃ (4.74 g, 44.76 mmol, 5.0 equiv) in dioxane (70 mL) and H₂O (35 mL) was added Pd(PPh₃)₄ (1.03 g, 895.11 μmol, 0.1 equiv). The reaction mixture was heated to 110° C. for 3 h, at which point the mixture was cooled to room temperature and poured into H₂O at 0° C. The precipitate was filtered, and the solid cake was dried under reduced pressure. The crude product was washed with EtOAc (50 mL) to afford the desired product as light yellow solid (3.14 g, 68% yield).

### Step 6: Synthesis of 5-(4-amino-1-((5,6,7,8-tetrahydropyrido[4,3-d]pyrimidin-2-yl)methyl)-1H-pyrazolo[3,4-d]pyrimidin-3-yl)benzo[d]oxazol-2-amine

A solution of tert-butyl 2-((4-amino-3-(2-aminobenzo[d]oxazol-5-yl)-1H-pyrazolo[3,4-d]pyrimidin-1-yl)methyl)-7,8-dihydropyrido[4,3-d]pyrimidine-6(5H)-carboxylate (3.14 g, 6.10 mmol, 1.0 equiv) in TFA (20 mL) was stirred at room temperature for 30 min. The mixture was concentrated under reduced pressure and the resulting residue was added dissolved in MeCN (7 mL) and added to MTBE (700 mL). The precipitate was collected by filtration to afford the desired product as a brown solid (4.25 g, 92% yield, 3 TFA). LCMS (ESI) m/z: [M+H] calcd for C₂₀H₁₈N₁₀O: 415.18; found 415.1.

### Monomer AG. 5-(4-amino-1-((2-((2-aminoethyl)sulfonyl)-1,2,3,4-tetrahydroisoquinolin-6-yl)methyl)-1H-pyrazolo[3,4-d]pyrimidin-3-yl)benzo[d]oxazol-2-amine

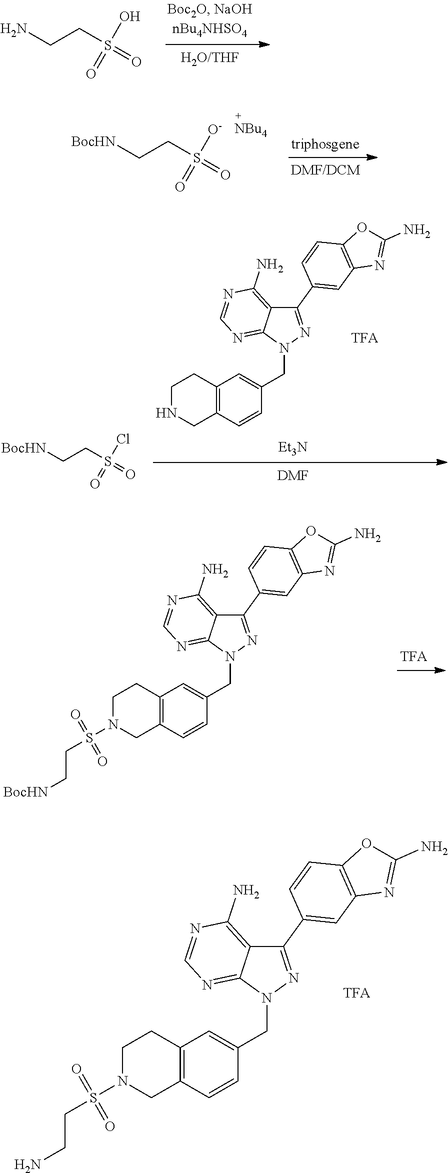

### Step 1: Synthesis of N-Boc taurine tetrabutylammonium salt

To a solution of 2-aminoethanesulfonic acid (10.00 mL, 79.91 mmol, 1.0 equiv) in THF (60 mL) and aqueous NaOH (2 M, 40 mL, 1.0 equiv) was added Boc₂O (18.31 g, 83.90 mmol, 1.05 equiv). The mixture was stirred at room temperature for 15 h, at which point the mixture was extracted with EtOAc (10 mL). The aqueous phase was diluted with H₂O (450 mL), treated with LiOH·H₂O (3.35 g, 79.83 mmol, 1.0 equiv) and nBu₄NHSO₄ (27.13 g 79.90 mmol, 1.0 equiv) and stirred for 30 min. This mixture was extracted with DCM (3×80 mL), and the combined organic phases were dried and concentrated under reduced pressure to afford the desired product as a colorless oil (34.26 g, 91% yield).

### Step 2: Synthesis of tert-butyl (2-(chlorosulfonyl)ethyl)carbamate

To a solution of N-Boc taurine tetrabutylammonium salt (4.7 g, 10.05 mmol, 1.0 equiv) in DCM (42 mL) was added DMF (77.32 μL, 1.00 mmol, 0.1 equiv) followed by a solution of triphosgene (0.5 M, 8.04 mL, 0.4 equiv) in DCM at 0° C. The mixture was warmed to room temperature and stirred for 30 min. The solution of tert-butyl (2-(chlorosulfonyl)ethyl)carbamate (2.45 g, crude) in DCM was used directly in the next step.

### Step 3: Synthesis of tert-butyl (2-((6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)sulfonyl)ethyl)carbamate

To a solution of 5-(4-amino-1-((1,2,3,4-tetrahydroisoquinolin-6-yl)methyl)-1H-pyrazolo[3,4-d]pyrimidin-3-yl)benzo[d]oxazol-2-amine (6.04 g, 9.44 mmol, 1.0 equiv, 2TFA) in DMF (40 mL) was added Et₃N (7.88 mL, 56.63 mmol, 6.0 equiv). A solution of tert-butyl (2-(chlorosulfonyl)ethyl)carbamate in DCM (42 mL) at 0° C. was added. The mixture was warmed to room temperature and stirred 16 h. The reaction mixture was concentrated under reduced pressure to remove DCM and the resulting solution was purified by reverse phase chromatography (15→45% MeCN/H₂O) to afford the desired product as a white solid (5.8 g, 83% yield, TFA). LCMS (ESI) m/z: [M+H] calcd for C₂₉H₃₃N₉O₅S: 620.24; found 620.3.

### Step 4: Synthesis of 5-(4-amino-1-((2-((2-aminoethyl)sulfonyl)-1,2,3,4-tetrahydroisoquinolin-6-yl)methyl)-1H-pyrazolo[3,4-d]pyrimidin-3-yl)benzo[d]oxazol-2-amine

A solution of tert-butyl (2-((6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)sulfonyl)ethyl)carbamate (5.8 g, 9.36 mmol, 1.0 equiv) in TFA (48 mL) was stirred at room temperature for 0.5 h, at which point the reaction mixture was concentrated under reduced pressure. The crude product dissolved in MeCN (30 mL) and was added dropwise into MTBE (200 mL). The mixture was stirred for 5 min and filtered, the filter cake was dried under reduced pressure to afford the desired product as a yellow solid (3.6 g, 62% yield, 2.2TFA). LCMS (ESI) m/z: [M+H] calcd for C₂₄H₂₅N₉O₃S: 520.19; found 520.1.

### Monomer AH. tert-butyl ((5-((4-amino-3-(2-aminobenzo[d]oxazol-5-yl)-1H-pyrazolo[3,4-d]pyrimidin-1-yl)methyl)pyrimidin-2-yl)methyl)carbamate

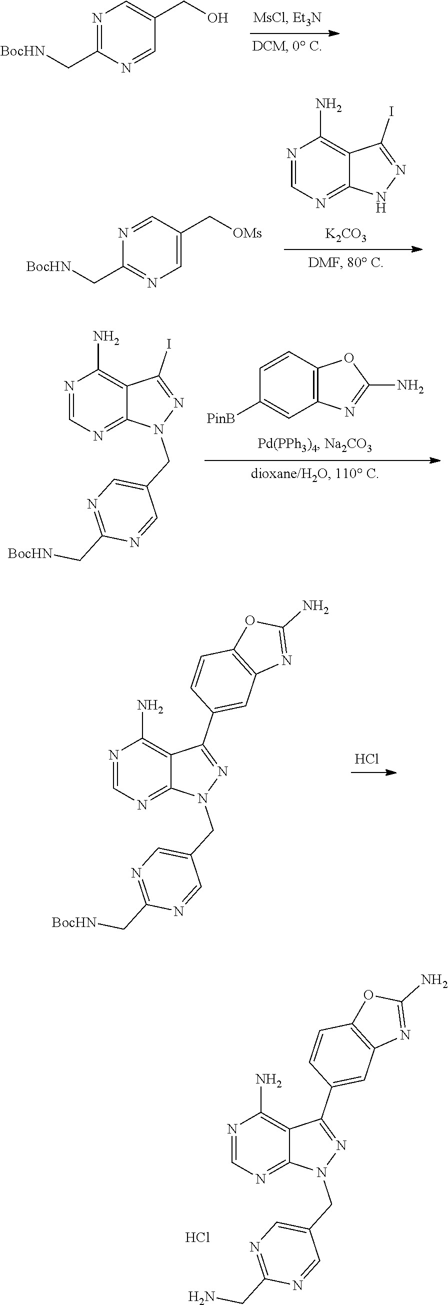

### Step 1: Synthesis of (2-(((tert-butoxycarbonyl)amino)methyl)pyrimidin-5-yl)methyl methanesulfonate

To a solution of tert-butyl ((5-(hydroxymethyl)pyrimidin-2-yl)methyl)carbamate (4.2 g, 17.55 mmol, 1.0 equiv) in DCM (42 mL) at 0° C. was added Et₃N (7.33 mL, 52.66 mmol, 3.0 equiv) followed by MsCl (2.41 g, 21.06 mmol, 1.63 mL, 1.2 equiv). The mixture was stirred at 0° C. for 10 min, and then H₂O (15 mL) was added. The reaction mixture was extracted with DCM (5×10 mL) and the combined organic phases were washed with brine (5 mL), dried, filtered, and concentrated under reduced pressure to afford the desired product (5.5 g, 98.7% yield) as a colorless solid.

### Step 2: Synthesis of tert-butyl ((5-((4-amino-3-iodo-1H-pyrazolo[3,4-d]pyrimidin-1-yl)methyl)pyrimidin-2-yl)methyl)carbamate

To a solution of (2-(((tert-butoxycarbonyl)amino)methyl)pyrimidin-5-yl)methyl methanesulfonate (5.47 g, 17.24 mmol, 1.2 equiv) and 3-iodo-1H-pyrazolo[3,4-d]pyrimidin-4-amine (3.75 g, 14.37 mmol, 1.0 equiv) in DMF (55 mL) at room temperature was added K₂CO₃ (5.96 g, 43.10 mmol, 3 equiv). The mixture was stirred at 80° C. for 5 h, at which point H₂O (100 mL) and brine (20 mL) were poured into the reaction mixture. The solution was extracted with EtOAc (10×30 mL) and the combined organic phases were dried, filtered, and concentrated under reduced pressure. Purification by silica gel chromatography (0→30% EtOAc/MeOH) afforded the desired product (2 g, 28.9% yield) as a yellow solid.

### Step 3: Synthesis of tert-butyl ((5-((4-amino-3-(2-aminobenzo[d]oxazol-5-yl)-1H-pyrazolo[3,4-d]pyrimidin-1-yl)methyl)pyrimidin-2-yl)methyl)carbamate

To a solution of tert-butyl ((5-((4-amino-3-iodo-1H-pyrazolo[3,4-d]pyrimidin-1-yl)methyl)pyrimidin-2-yl)methyl)carbamate (2 g, 4.15 mmol, 1.0 equiv), 5-(4,4,5,5-tetramethyl-1,3,2-dioxaborolan-2-yl)-1,3-benzoxazol-2-amine (1.13 g, 4.35 mmol, 1.05 equiv) and Na₂CO₃ (688.39 mg, 8.29 mmol, 2.0 equiv) in dioxane (20 mL) and H₂O (10 mL) was added Pd(PPh₃)₄ (479.21 mg, 414.70 μmol, 0.1 equiv). The mixture was stirred at 110° C. for 1 h, at which time the mixture was cooled to room temperature, filtered, and the solid cake washed with MeOH (3×10 mL). The filtrate was concentrated under reduced pressure to remove MeOH and then added dropwise into H₂O (50 mL). The resulting suspension was filtered, and the filter cake was washed with H₂O (3×10 mL). The solid cake was stirred in MeOH (20 mL) for 30 min. The resulting suspension was filtered, and the filter cake washed with MeOH (3×8 mL). The filter cake was dried under reduced pressure to afford the desired product (1.03 g, 48.9% yield) as a white solid. LCMS (ESI) m/z: [M+H] calcd for C₂₃H₂₄N₁₀O₃: 489.21; found 489.2.

### Step 4: Synthesis of 5-(4-amino-1-{[2-(aminomethyl)pyrimidin-5-yl]methyl}-1H-pyrazolo[3,4-d]pyrimidin-3-yl)-1,3-benzoxazol-2-amine

To tert-butyl ((5-((4-amino-3-(2-aminobenzo[d]oxazol-5-yl)-1H-pyrazolo[3,4-d]pyrimidin-1-yl)methyl)pyrimidin-2-yl)methyl)carbamate (100 mg, 0.205 mmol, 1.0 equiv) was added con. HCl (850 μL, 10.2 mmol, 50 equiv). The reaction was stirred for 1 h and was then poured into acetone (3 mL). The resulting precipitate was filtered, washed with acetone, and dried under reduced pressure to afford the desired product (80 mg, 92% yield) as a brown solid. LCMS (ESI) m/z: [M+H] calcd for C₁₈H₁₆N₁₀O: 389.16; found 389.0.

### Monomer AI. 5-(4-(dimethylamino)-1-((1,2,3,4-tetrahydroisoquinolin-6-yl)methyl)-1H-pyrazolo[3,4-d]pyrimidin-3-yl)benzo[d]oxazol-2-amine trifluoroacetic acid salt

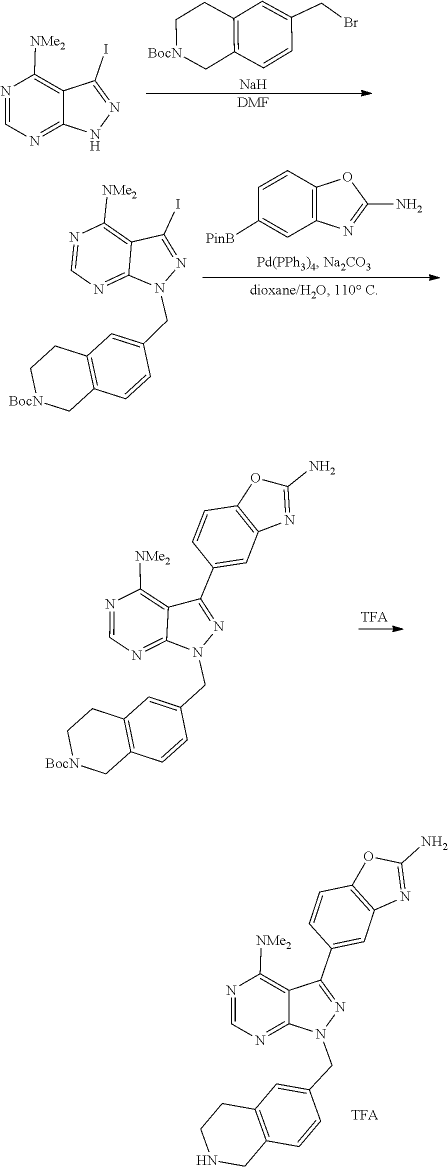

### Step 1: Synthesis of tert-butyl 6-((4-(dimethylamino)-3-iodo-1H-pyrazolo[3,4-d]pyrimidin-1-yl)methyl)-3,4-dihydroisoquinoline-2(1H)-carboxylate

To a solution of 3-iodo-N,N-dimethyl-1H-pyrazolo[3,4-d]pyrimidin-4-amine (3.6 g, 12.45 mmol, 1.0 equiv) in DMF (36 mL) at 0° C. was added NaH (523.00 mg, 13.08 mmol, 60 wt. %, 1.05 equiv). The mixture was stirred at 0° C. for 30 min. To the reaction mixture was then added a solution of tert-butyl 6-(bromomethyl)-3,4-dihydroisoquinoline-2(1H)-carboxylate (4.47 g, 13.70 mmol, 1.1 equiv) in DMF (18 mL) at 0° C. The mixture was stirred at room temperature for 2 h. The reaction mixture was then added to cold H₂O (200 mL) and stirred for 30 min. The resulting precipitate was collected by filtration to afford the desired product (6 g, 71.9% yield) as a white solid.

### Step 2: Synthesis of tert-butyl 6-((3-(2-aminobenzo[d]oxazol-5-yl)-4-(dimethylamino)-1H-pyrazolo[3,4-d]pyrimidin-1-yl)methyl)-3,4-dihydroisoquinoline-2(1H)-carboxylate

To a solution of tert-butyl 6-((4-(dimethylamino)-3-iodo-1H-pyrazolo[3,4-d]pyrimidin-1-yl)methyl)-3,4-dihydroisoquinoline-2(1H)-carboxylate (2 g, 2.96 mmol, 1.0 equiv) and 5-(4,4,5,5-tetramethyl-1,3,2-dioxaborolan-2-yl)benzo[d]oxazol-2-amine (922.81 mg, 3.55 mmol, 1.2 equiv) in dioxane (24 mL) and H₂O (12 mL) was added Na₂CO₃ (1.57 g, 14.78 mmol, 5.0 equiv) and Pd(PPh₃)₄ (341.66 mg, 295.66 μmol, 0.1 equiv). The mixture was stirred at 110° C. for 12 h. The reaction mixture was then poured into cold H₂O (200 mL) and stirred for 30 min. The resulting precipitate was collected by filtration. Purification by silica gel chromatography (5→100% petroleum ether/EtOAc) afforded the desired product (1.2 g, 72.3% yield) as a yellow solid.

### Step 3: Synthesis of 5-(4-(dimethylamino)-1-((1,2,3,4-tetrahydroisoquinolin-6-yl)methyl)-1H-pyrazolo[3,4-d]pyrimidin-3-yl)benzo[d]oxazol-2-amine

A solution of tert-butyl 6-((3-(2-aminobenzo[d]oxazol-5-yl)-4-(dimethylamino)-1H-pyrazolo[3,4-d]pyrimidin-1-yl)methyl)-3,4-dihydroisoquinoline-2(1H)-carboxylate (1.7 g, 3.14 mmol, 1.0 equiv) in TFA (10 mL) was stirred at room temperature for 30 min. The reaction mixture was then concentrated under reduced pressure. The residue was added to MeCN (10 mL) and the solution was added dropwise into MTBE (200 mL). The resulting solid was dissolved in MeCN (30 mL) and the solution was concentrated under reduced pressure to afford the desired product (1.67 g, 92.9% yield,) as a yellow solid. LCMS (ESI) m/z: [M+H] calcd for C₂₄H₂₄N₈O: 441.22; found 441.2.

### Monomer AJ. 4-amino-5-(2-aminobenzo[d]oxazol-5-yl)-5H-pyrimido[5,4-b]indole-7-carboxylic acid

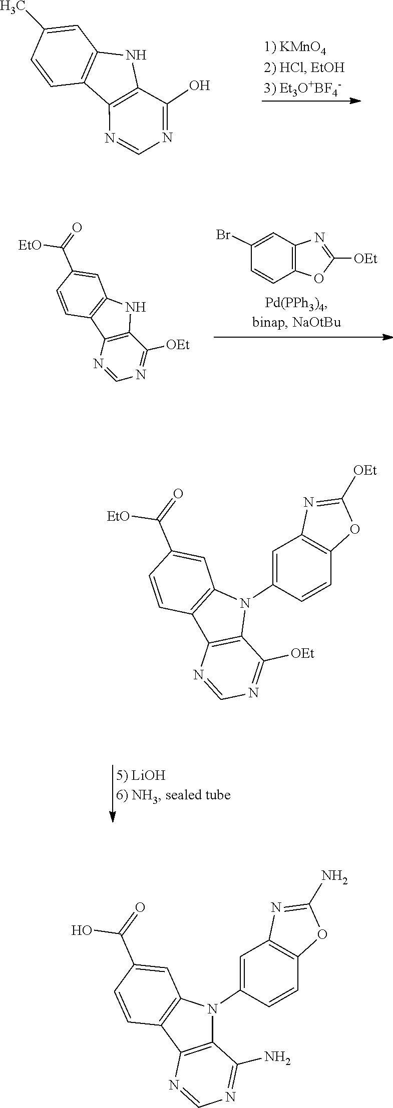 [SMILES: Cc1ccc2c(c1)[nH]c1c(O)ncnc12 ; CCOC(=O)c1ccc2c(c1)[nH]c1c(OCC)ncnc12 ; CCOc1nc2cc(Br)ccc2o1 ; CCOC(=O)c1ccc2c3ncnc(OCC)c3n(-c3ccc4oc(OCC)nc4c3)c2c1 ; Nc1nc2cc(-n3c4cc(C(=O)O)ccc4c4ncnc(N)c43)ccc2o1]

This monomer can be prepared from 7-methyl-5H-pyrimido[5,4-b]indol-4-ol by benzylic oxidation to the carboxylic acid, conversion to the ethyl ester, followed by O-ethylation with triethyloxonium tetrafluoroboroate. Palladium-mediated arylation followed by ester hydrolysis and final ammonia-olysis provides the monomer.

### Monomer AK. 4-amino-5-(2-aminobenzo[d]oxazo-5-yl)-5H-pyrimido[5,4-b]indole-8-carboxylic acid

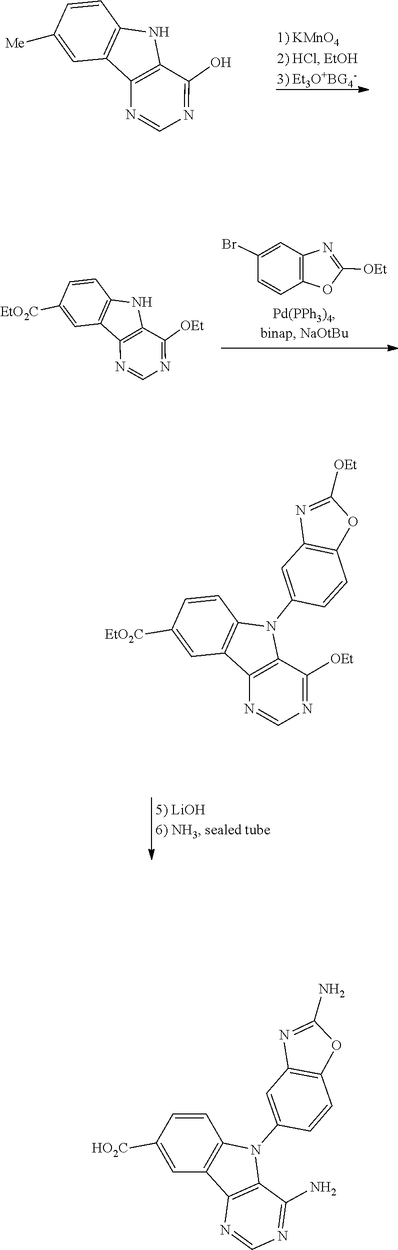 [SMILES: Cc1ccc2[nH]c3c(O)ncnc3c2c1 ; CCOC(=O)c1ccc2[nH]c3c(OCC)ncnc3c2c1 ; CCOc1nc2cc(Br)ccc2o1 ; CCOC(=O)c1ccc2c(c1)c1ncnc(OCC)c1n2-c1ccc2oc(OCC)nc2c1 ; Nc1nc2cc(-n3c4ccc(C(=O)O)cc4c4ncnc(N)c43)ccc2o1]

This monomer can be prepared following a similar route as that to prepare the previous monomer, but using the isomeric starting material from 8-methyl-5H-pyrimido[5,4-b]indol-4-ol. Benzylic oxidation to the carboxylic acid, conversion to the ethyl ester, followed by O-ethylation with triethyloxonium tetrafluoroboroate and palladium-mediated arylation, followed by ester hydrolysis and final ammonia-olysis provides the monomer.

### Monomer AL. 3-(2,4-bis((S)-3-methylmorpholino)-4a,8a-dihydropyrido[2,3-d]pyrimidin-7-yl)benzoic acid

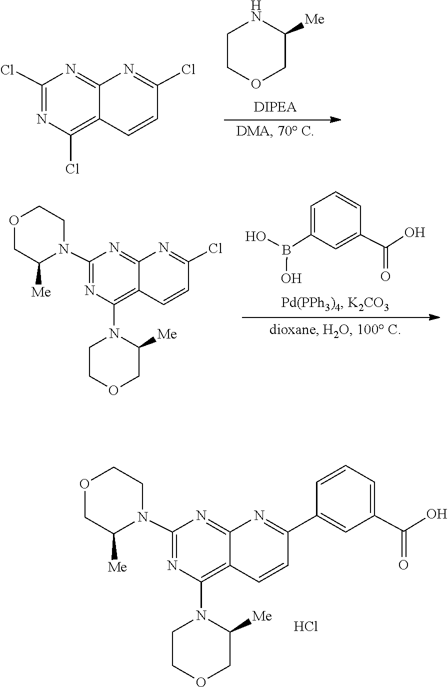

### Step 1: Synthesis of (3S)-4-[7-chloro-2-[(3S)-3-methylmorpholin-4-yl]pyrido[2,3-d]pyrimidin-4-yl]3-methyl-morpholine

To a solution of 2,4,7-trichloropyrido[2,3-d]pyrimidine (4.0 g, 17.06 mmol, 1.0 equiv) in DMA (10 mL) was added (3S)-3-methylmorpholine (4.31 g, 42.65 mmol, 2.5 equiv) and DIPEA (5.51 g, 42.65 mmol, 7.43 mL, 2.5 equiv). The reaction solution was heated to 70° C. for 48 h. The reaction suspension was cooled to room temperature, poured into cold H₂O (50 mL) to precipitate out a solid. The solid was filtered and the filter cake was rinsed with H₂O, and dried under reduced pressure to give the crude product, which was purified by column chromatography on silica gel (0→100% petroleum ether/EtOAc) to give (3S)-4-[7-chloro-2-[(3S)-3-methylmorpholin-4-yl]pyrido[2,3-d]pyrimidin-4-yl]3-methyl-morpholine (3.5 g, 56.4% yield) as a yellow solid. LCMS (ESI) m/z: [M+H] calcd for C₁₇H₂₂ClN₅O₂: 364.15; found 364.2.

### Step 2: Synthesis of 3-[2,4-bis[(3S)-3-methylmorpholin-4-yl]pyrido[2,3-d]pyrimidin-7-yl]benzoic acid

To a solution of (3S)-4-[7-chloro-2-[(3S)-3-methylmorpholin-4-yl]pyrido[2,3-d]pyrimidin-4-yl]-3-methyl-morpholine (2 g, 5.50 mmol, 1.0 equiv) and 3-boronobenzoic acid (1.09 g, 6.60 mmol, 1.2 equiv) in 1,4-dioxane (40 mL) was added a solution of K₂CO₃ (911.65 mg, 6.60 mmol, 1.2 equiv) in H₂O (4 mL), followed by Pd(PPh₃)₄ (317.60 mg, 274.85 μmol, 0.05 equiv). The solution was degassed for 10 min and refilled with N₂, then the reaction mixture was heated to 100° C. under N₂ for 5 h. The reaction was cooled to room temperature and filtered. The filtrate was acidified by HCl (2N) to pH 3, and the aqueous layer was washed with EtOAc (3×20 mL). The aqueous phase was concentrated under reduced pressure to give a residue, which was purified by column chromatography on silica gel (50%→100% petroleum ether/EtOAc) to give 3-[2,4-bis[(3S)-3-methylmorpholin-4-yl]pyrido[2,3-d]pyrimidin-7-yl]benzoic acid hydrochloride (2.5 g, 89.9% yield) as a yellow solid. LCMS (ESI) m/z: [M+H] calcd for C₂₄H₂₇N₅O₄: 450.21; found 450.2.

Reference for preparation of this monomer: Menear, K.; Smith, G. C. M.; Malagu, K.; Duggan, H. M. E.; Martin, N. M. B.; Leroux, F. G. M. 2012. Pyrido-, pyrazo- and pyrimido-pyrimidine derivatives as mTOR inhibitors. U.S. Pat. No. 8,101,602. Kudos Pharmaceuticals, Ltd, which is incorporated by reference in its entirety.

### Monomer AM. (1r,4r)-4-[4-amino-5-(7-methoxy-1H-indol-2-yl)imidazo[4,3-f][1,2,4]triazin-7-yl]cyclohexane-1-carboxylic acid

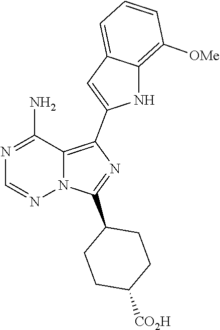 [SMILES: COc1cccc2cc(-c3nc([C@H]4CC[C@H](C(=O)O)CC4)n4ncnc(N)c34)[nH]c12]

This monomer, also known as OSI-027 (CAS #=936890-98-1), is a commercially available compound. At the time this application was prepared, it was available for purchase from several vendors.

### Monomer AN. 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)pyrimidine-5-carboxylic acid

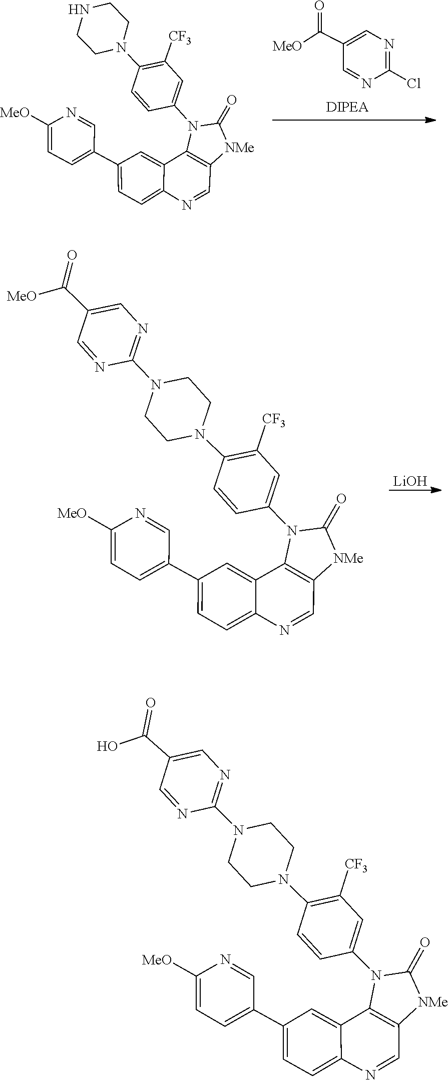

Preparation of this monomer proceeds by reaction of BGT226 with methyl 2-chloropyrimidine-5-carboxylate, followed by ester hydrolysis, to give the titled Monomer.

### Monomer AO. 4-amino-5-{1H-pyrrolo[2,3-b]pyridin-5-yl}-5H-pyrimido[5,4-b]indole-8-carboxylic acid

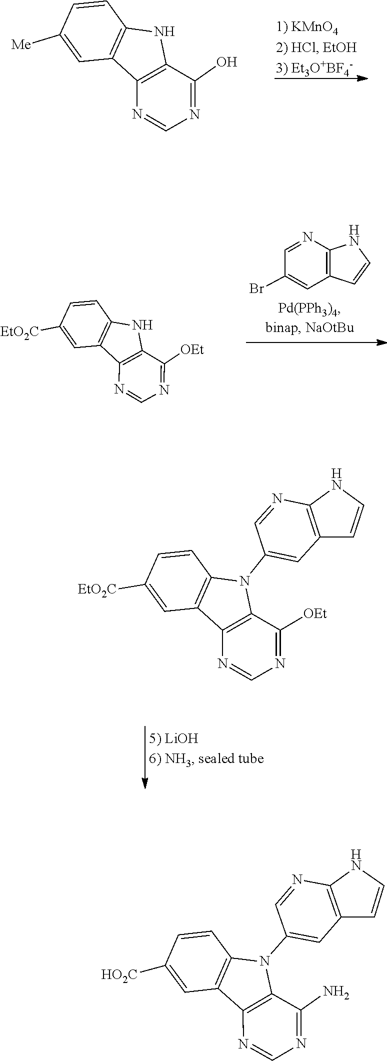 [SMILES: Cc1ccc2[nH]c3c(O)ncnc3c2c1 ; Brc1cnc2[nH]ccc2c1 ; CCOC(=O)c1ccc2[nH]c3c(OCC)ncnc3c2c1 ; CCOC(=O)c1ccc2c(c1)c1ncnc(OCC)c1n2-c1cnc2[nH]ccc2c1 ; Nc1ncnc2c3cc(C(=O)O)ccc3n(-c3cnc4[nH]ccc4c3)c12]

This monomer can be prepared from 7-methyl-5H-pyrimido[5,4-b]indol-4-ol by benzylic oxidation to the carboxylic acid, conversion to the ethyl ester, followed by O-ethylation with triethyloxonium tetrafluoroboroate. Palladium-mediated arylation followed by ester hydrolysis and final ammonia-olysis provides the monomer.

### Preparation of Pre- and Post-Linkers

### Building Block A. tert-butyl N-[(tert-butoxy)carbonyl]-N-{[2-(piperazin-1-yl)pyrimidin-5-yl]methyl}carbamate

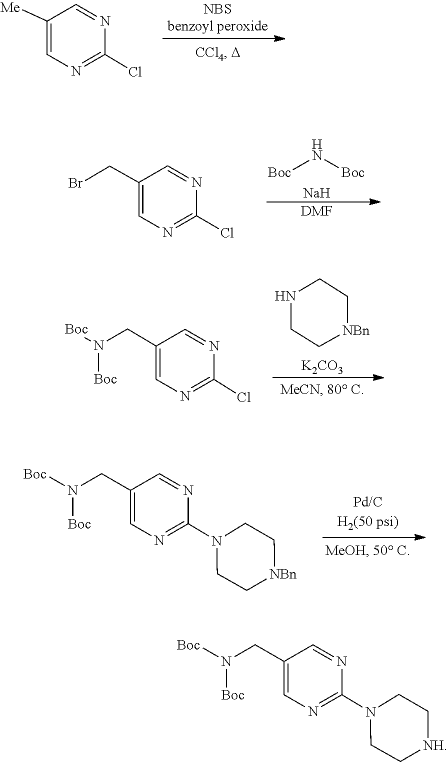

### Step 1: Synthesis of 5-(bromomethyl)-2-chloropyrimidine

To a solution of 2-chloro-5-methylpyrimidine (92 g, 715.62 mmol, 1.0 equiv) in CCl₄ (1000 mL) was added NBS (178.31 g, 1.00 mol, 1.4 equiv) and benzoyl peroxide (3.47 g, 14.31 mmol, 0.02 equiv). The mixture was stirred at 76° C. for 18 h. The reaction mixture was then cooled to room temperature and concentrated under reduced pressure. The reaction mixture was filtered and the solid cake was washed with DCM (150 mL). The resulting solution was concentrated under reduced pressure to give the crude product. The residue was purified by silica gel chromatography (1/0 to 0/1 petroleum ether/EtOAc) to give the product (70.8 g, 47.7% crude yield) as yellow oil, which was used directly for the next step. LCMS (ESI) m/z: [M+H] calcd for C₅H₄BrClN₂: 206.93; found 206.9.

### Step 2: Synthesis of tert-butyl N-tert-butoxycarbonyl-N-((2-piperazin-1-ylpyrimidin-5-yl)methyl)carbamate

To a solution of tert-butyl N-tert-butoxycarbonylcarbamate (36.89 g, 169.79 mmol, 0.74 equiv) in DMF (750 mL) was added NaH (6.88 g, 172.09 mmol, 60 wt. %, 0.75 equiv) at 0° C. The mixture was stirred at 0° C. for 30 min. Then, 5-(bromomethyl)-2-chloro-pyrimidine (47.6 g, 229.45 mmol, 1.0 equiv) was added at 0° C. The reaction mixture was stirred at room temperature for 15.5 h. The mixture was then poured into H₂O (1600 mL) and the aqueous phase was extracted with EtOAc (3×300 mL). The combined organic phase was washed with brine (2×200 mL), dried with anhydrous Na₂SO₄, filtered and concentrated under reduced pressure. The residue was purified by silica gel chromatography (1/0 to 0/1 petroleum ether/EtOAc) to give the product (70 g, crude) as a yellow solid, which was used to next step directly.

### Step 3: Synthesis of tert-butyl N-tert-butoxycarbonyl-N-[(2-piperazin-1-ylpyrimidin-5-yl)methyl]carbamate

To a solution of 1-benzylpiperazine (30.44 g, 122.16 mmol, 1.0 equiv, 2HCl) in MeCN (550 mL) was added tert-butyl N-tert-butoxycarbonyl-N-((2-chloropyrimidin-5-yl)methyl)carbamate (42 g, 122.16 mmol, 1.0 equiv) and K₂CO₃ (84.42 g, 610.81 mmol, 5.0 equiv). The mixture was stirred at 80° C. for 61 h. The reaction mixture was then diluted with EtOAc (150 mL) and the mixture was filtered. The resulting solution was concentrated under reduced pressure to give the crude product. The residue was purified by silica gel chromatography (1/0 to 0/1 petroleum ether/EtOAc) to give the product (45 g, 74% yield) as a white solid.

### Step 4: Synthesis of tert-butyl N-tert-butoxycarbonyl-N-[(2-piperazin-1-ylpyrimidin-5-yl)methyl]carbamate

To a solution of tert-butyl N-[[2-(4-benzylpiperazin-1-yl)pyrimidin-5-yl]methyl]-N-tert-butoxycarbonyl-carbamate (24 g, 49.63 mmol, 1.0 equiv) in MeOH (600 mL) was added Pd/C (24 g, 47.56 mmol, 10 wt. %, 1.0 equiv) under argon. The mixture was degassed under reduced pressure and purged with H₂ three times. The mixture was stirred under H₂ (50 psi) at 50° C. for 19 h. The reaction mixture was cooled to room temperature, filtered, and the filter cake was washed with MeOH (500 mL). The resulting solution was concentrated under reduced pressure. The residue was purified by silica gel chromatography (1/0 to 0/1 EtOAc/MeOH) to give the product (25.5 g, 68% yield) as a white solid.

### Building Block B. 2-(4-(5-(((tert-butoxycarbonyl)amino)methyl)pyrimidin-2-yl)piperazin-1-yl)pyrimidine-5-carboxylic acid

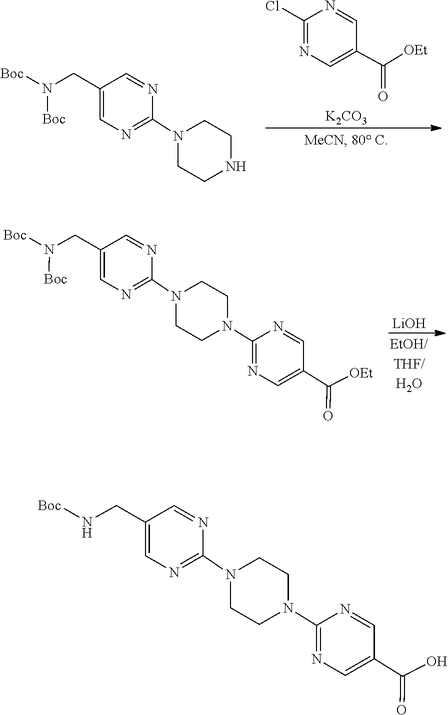 [SMILES: CC(C)(C)OC(=O)N(Cc1cnc(N2CCNCC2)nc1)C(=O)OC(C)(C)C ; "K2CO3" ; CCOC(=O)c1cnc(Cl)nc1 ; CCOC(=O)c1cnc(N2CCN(c3ncc(CN(C(=O)OC(C)(C)C)C(=O)OC(C)(C)C)cn3)CC2)nc1 ; "LiOH" ; CC(C)(C)OC(=O)NCc1cnc(N2CCN(c3ncc(C(=O)O)cn3)CC2)nc1]

### Step 1: Synthesis of ethyl 2-(4-(5-((bis(tert-butoxycarbonyl)amino)methyl)pyrimidin-2-yl)piperazin-1-yl)pyrimidine-5-carboxylate

To a solution of ethyl 2-chloropyrimidine-5-carboxylate (2.37 g, 12.71 mmol, 1.0 equiv) and tert-butyl N-tert-butoxycarbonyl-N-((2-piperazin-1-ylpyrimidin-5-yl)methyl)carbamate (5 g, 12.71 mmol, 1.0 equiv) in MeCN (80 mL) was added K₂CO₃ (5.27 g, 38.12 mmol, 3.0 equiv). The mixture was stirred at 80° C. for 16 h. The reaction mixture was then poured into H₂O (200 mL) and the suspension was filtered. The filtrate was washed with H₂O (80 mL) and dried under reduced pressure to give the product (6.1 g, 87% yield) as a white solid.

### Step 2: Synthesis of 2-(4-(5-(((tert-butoxycarbonyl)amino)methyl)pyrimidin-2-yl)piperazin-1-yl)pyrimidine-5-carboxylic acid

To a solution of ethyl 2-(4-(5-((bis(tert-butoxycarbonyl)amino)methyl)pyrimidin-2-yl)piperazin-1-yl)pyrimidine-5-carboxylate (5 g, 9.20 mmol, 1.0 equiv) in H₂O (50 mL), EtOH (15 mL) and THF (50 mL) was added LiOH·H₂O (1.54 g, 36.79 mmol, 4.0 equiv). The reaction mixture was stirred at 55° C. for 16 h. The mixture was then concentrated to remove THF and EtOH and then the mixture was diluted with H₂O (55 mL) and was acidified (pH=3) with aqueous HCl (1 N). The mixture was filtered and the filter cake was washed with H₂O (36 mL). The filter cake was dried under reduced pressure to give the product (2.7 g, 69.3%) as a white solid. LCMS (ESI) m/z: [M+H] calcd for C₁₉H₂₅N₇O₄: 416.21; found 416.1.

### Building Block C. tert-butyl 2-(piperazin-1-yl)-7,8-dihydropyrido[4,3-d]pyrimidine-6(5H)-carboxylate

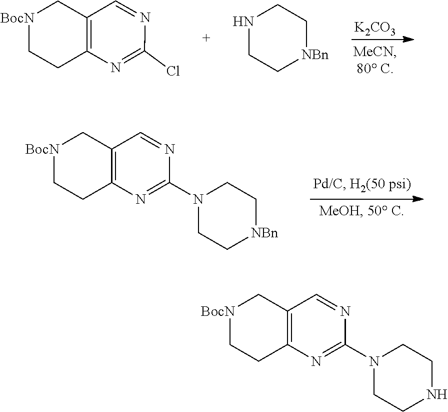

### Step 1: Synthesis of tert-butyl 2-(4-benzylpiperazin-1-yl)-7,8-dihydropyrido[4,3-d]pyrimidine-6(5H)-carboxylate

To a solution of tert-butyl 2-chloro-7,8-dihydropyrido[4,3-d]pyrimidine-6(5H)-carboxylate (15 g, 55.61 mmol, 1.0 equiv) in MeCN (150 mL) was added 1-benzylpiperazine (11.76 g, 66.73 mmol, 1.2 equiv) and K₂CO₃ (46.12 g, 333.67 mmol, 6.0 equiv). The mixture was stirred at 80° C. for 27 h. The reaction mixture was diluted with EtOAc (200 mL), filtered and concentrated under reduced pressure. The crude product was purified by silica gel chromatography (1/0 to 0/1 petroleum ether/EtOAc) to give the product (20.2 g, 80% yield) as a white solid. LCMS (ESI) m/z: [M+H] calcd for C₂₃H₃₁N₅O₂: 410.26; found 410.1.

### Step 2: Synthesis of tert-butyl 2-(piperazin-1-yl)-7,8-dihydropyrido[4,3-d]pyrimidine-6(5H)-carboxylate

To a solution of tert-butyl 2-(4-benzylpiperazin-1-yl)-7,8-dihydropyrido[4,3-d]pyrimidine-6(5H)-carboxylate (8 g, 19.53 mmol, 1.0 equiv) in MeOH (200 mL) was added Pd/C (8 g, 19.53 mmol, 10 wt. %, 1.0 equiv) under argon. The mixture was degassed and purged with H₂ three times. The mixture was stirred under H₂ (50 psi) at 50° C. for 19 h. The reaction mixture was cooled to room temperature, filtered through a pad of Celite and the filter cake was washed with MeOH (150 mL). The resulting solution was concentrated under reduced pressure. The crude product was washed with petroleum ether (60 mL) to give the product (9.25 g, 72% yield) as a white solid. LCMS (ESI) m/z: [M+H] calcd for C₁₆H₂₅N₅O₂: 320.21; found 320.2.

### Building Block D. 2-(4-(6-(tert-butoxycarbonyl)-5,6,7,8-tetrahydropyrido[4,3-d]pyrimidin-2-yl)piperazin-1-yl)pyrimidine-5-carboxylic acid

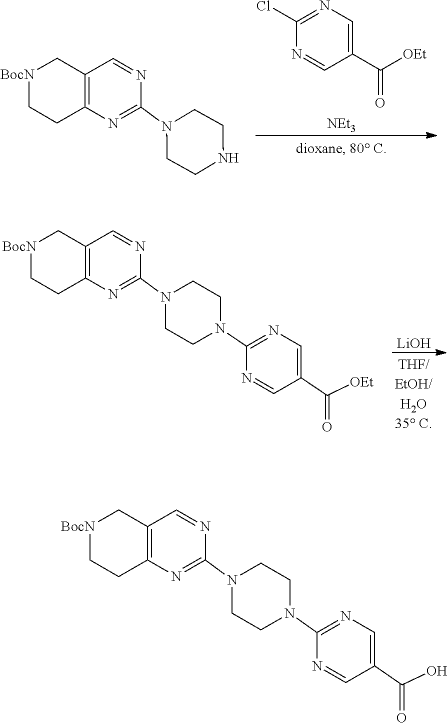 [SMILES: CC(C)(C)OC(=O)N1CCc2nc(N3CCNCC3)ncc2C1 ; CCOC(=O)c1cnc(Cl)nc1 ; CCOC(=O)c1cnc(N2CCN(c3ncc4c(n3)CCN(C(=O)OC(C)(C)C)C4)CC2)nc1 ; "LiOH" ; CC(C)(C)OC(=O)N1CCc2nc(N3CCN(c4ncc(C(=O)O)cn4)CC3)ncc2C1 ; "NEt3"]

### Step 1: Synthesis of tert-butyl 2-(4-(5-(ethoxycarbonyl)pyrimidin-2-yl)piperazin-1-yl)-7,8-dihydropyrido[4,3-d]pyrimidine-6(5H)-carboxylate

To a solution of ethyl 2-chloropyrimidine-5-carboxylate (4.09 g, 21.92 mmol, 1.0 equiv) in dioxane (80 mL) was added tert-butyl 2-(piperazin-1-yl)-7,8-dihydropyrido[4,3-d]pyrimidine-6(5H)-carboxylate (7 g, 21.92 mmol, 1.0 equiv) and Et₃N (9.15 mL, 65.75 mmol, 3.0 equiv). The mixture was stirred at 90° C. for 64 h. The solution was poured into H₂O (200 mL) and then the mixture was filtered and the filter cake was washed with H₂O (100 mL) followed by petroleum ether (60 mL). The filter cake was dried under reduced pressure to give the product (10.1 g, 92% yield) as a brown solid. LCMS (ESI) m/z: [M+H] calcd for C₂₃H₃₁N₇O₄: 470.25; found 470.4.

### Step 2: Synthesis of 2-(4-(6-(tert-butoxycarbonyl)-5,6,7,8-tetrahydropyrido[4,3-d]pyrimidin-2-yl)piperazin-1-yl)pyrimidine-5-carboxylic acid

To a solution of tert-butyl 2-(4-(5-(ethoxycarbonyl)pyrimidin-2-yl)piperazin-1-yl)-7,8-dihydropyrido[4,3-d]pyrimidine-6(5H)-carboxylate (6.0 g, 12.78 mmol, 1.0 equiv) in THF (40 mL), EtOH (20 mL) and H₂O (40 mL) was added LiOH·H₂O (1.07 g, 25.56 mmol, 2.0 equiv). The reaction mixture was stirred at 35° C. for 15 h. The mixture was then concentrated under reduced pressure to remove THF and EtOH. The mixture was then diluted with H₂O (500 mL) and was adjusted to pH 3 with aqueous HCl (1 N). The mixture was filtered and the filter cake was washed with H₂O (80 mL) followed by petroleum ether (80 mL). The filter cake was dried under reduced pressure to give the product (3.8 g, 65% yield) as a white solid. LCMS (ESI) m/z: [M+H] calcd for C₂₁H₂₇N₇O₄: 442.22; found 442.3.

### Building Block E. tert-butyl methyl((2-(piperazin-1-yl)pyrimidin-5-yl)methyl)carbamate

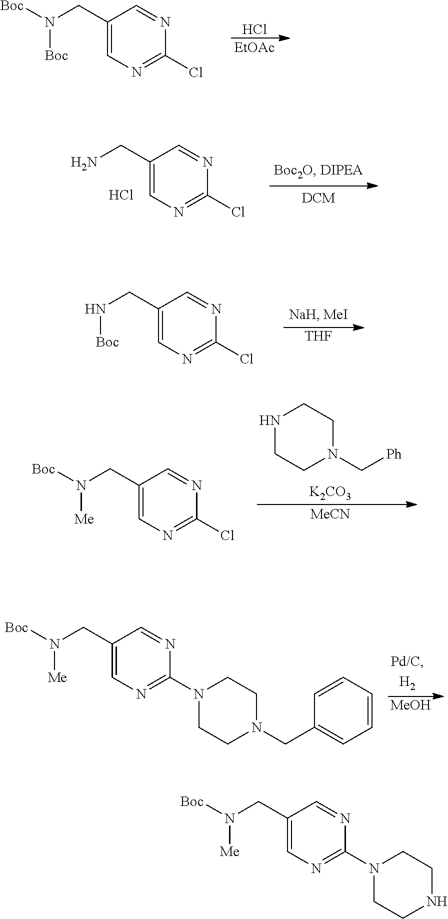

### Step 1: Synthesis of (2-chloropyrimidin-5-yl)methanamine

To a solution of tert-butyl N-tert-butoxycarbonyl-N-((2-chloropyrimidin-5-yl)methyl)carbamate (28 g, 81.44 mmol, 1.0 equiv) in EtOAc (30 mL) was added HCl in EtOAc (260 mL). The reaction mixture was stirred at room temperature for 5 h. The reaction mixture was filtered and the filter cake was washed with EtOAc (100 mL). The solid cake was dried under reduced pressure to give the product (14.3 g, 96.6% yield, HCl) as a white solid.

### Step 2: Synthesis of tert-butyl ((2-chloropyrimidin-5-yl)methyl)carbamate

To a solution of (2-chloropyrimidin-5-yl)methanamine (13 g, 72.21 mmol, 1.0 equiv, HCl) in DCM (130 mL) was added DIPEA (20.41 mL, 144.42 mmol, 1.8 equiv) and Boc₂O (16.59 mL, 72.21 mmol, 1.0 equiv), then the mixture was stirred at room temperature for 3 h. The reaction mixture was added to H₂O (100 mL) and then the aqueous layer was separated and extracted with DCM (2×100 mL). Then combined organic phase was washed with sat. NH₄Cl (2×200 mL) and brine (2×200 mL), dried with anhydrous Na₂SO₄, filtered and concentrated under reduced pressure. The residue was purified by silica gel chromatography (1/0 to 1/1 petroleum ether/EtOAc) to give the product (12 g, 68.2% yield) as a white solid.

### Step 3: Synthesis of tert-butyl ((2-chloropyrimidin-5-yl)methyl)(methyl)carbamate

To a solution of tert-butyl ((2-chloropyrimidin-5-yl)methyl)carbamate (11 g, 45.14 mmol, 1.0 equiv) and MeI (14.05 mL, 225.70 mmol, 5.0 equiv) in THF (150 mL) was added NaH (1.99 g, 49.65 mmol, 60 wt. %, 1.1 equiv) at 0° C. The mixture was stirred at 0° C. for 3 h and then the reaction was quenched with H₂O (100 mL). The aqueous phase was extracted with EtOAc (3×150 mL) and the combined organic phase was washed with brine (50 mL), dried with anhydrous Na₂SO₄, filtered and concentrated under reduced pressure. The residue was purified by silica gel chromatography (1/0 to 3/1 petroleum ether/EtOAc) to give the product (9 g, 77.4% yield) as a white solid.

### Step 4: Synthesis of tert-butyl ((2-(4-benzylpiperazin-1-yl)pyrimidin-5-yl)methyl)(methyl)carbamate

To a solution of tert-butyl ((2-chloropyrimidin-5-yl)methyl)(methyl)carbamate (9 g, 34.92 mmol, 1.0 equiv) in MeCN (90 mL) was added 1-benzylpiperazine (8.70 g, 34.92 mmol, 1.0 equiv, 2HCl), and K₂CO₃ (24.13 g, 174.61 mmol, 5.0 equiv). The reaction mixture was stirred at 80° C. for 20 h. The mixture was then filtered and concentrated under reduced pressure. The residue was purified by silica gel chromatography (1/0 to 1/1 petroleum ether/EtOAc) to give the product (12 g, 86.4% yield) as a yellow oil.

### Step 5: Synthesis of tert-butyl methyl((2-(piperazin-1-yl)pyrimidin-5-yl)methyl)carbamate

To a solution of tert-butyl ((2-(4-benzylpiperazin-1-yl)pyrimidin-5-yl)methyl)(methyl)carbamate (12 g, 30.19 mmol, 1.0 equiv) in MeOH (120 mL) was added Pd/C (2 g, 10 wt. %). The suspension was degassed and purged with H₂ and then the mixture was stirred under H₂ (15 psi) at room temperature for 3 h. The reaction mixture was filtered through Celite and the filtrate was concentrated under reduced pressure. The residue was purified by silica gel chromatography 1/0 to 1/1 petroleum ether/EtOAc) to give semi-pure material (9 g) as a yellow oil. Petroleum ether was added to the residue and the solution was stirred at −60° C. until solid appeared. The suspension was filtered and the filtrate was concentrated under reduced pressure to give the product (4.07 g, 55.6% yield) as a yellow oil. LCMS (ESI) m/z: [M+H] calcd for C₁₅H₂₅N₅O₂: 308.21; found 308.1.

### Building Block F. 2-(4-(5-(((tert-butoxycarbonyl)(methyl)amino)methyl) pyrimidin-2-yl)piperazin-1-yl)pyrimidine-5-carboxylic acid

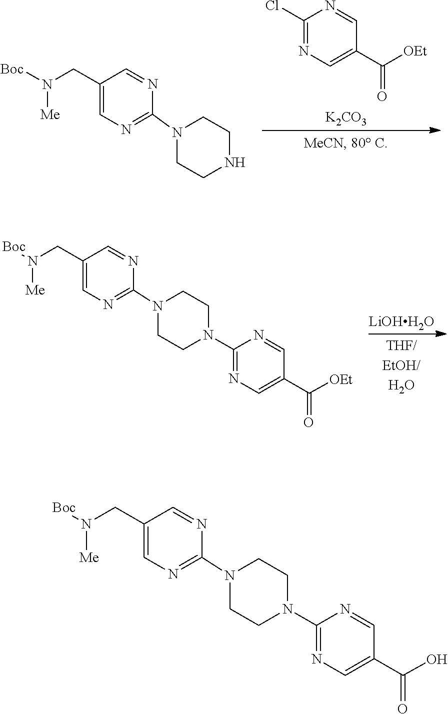 [SMILES: CN(Cc1cnc(N2CCNCC2)nc1)C(=O)OC(C)(C)C ; "K2CO3" ; CCOC(=O)c1cnc(Cl)nc1 ; CCOC(=O)c1cnc(N2CCN(c3ncc(CN(C)C(=O)OC(C)(C)C)cn3)CC2)nc1 ; "LiOH·H2O" ; CN(Cc1cnc(N2CCN(c3ncc(C(=O)O)cn3)CC2)nc1)C(=O)OC(C)(C)C]

### Step 1: Synthesis of ethyl 2-(4-(5-(((tert-butoxycarbonyl)(methyl)amino)methyl) pyrimidin-2-yl)piperazin-1-yl)pyrimidine-5-carboxylate

To a mixture of tert-butyl methyl((2-(piperazin-1-yl)pyrimidin-5-yl)methyl)carbamate (4.3 g, 13.99 mmol, 1.0 equiv) and ethyl 2-chloropyrimidine-5-carboxylate (2.87 g, 15.39 mmol, 1.1 equiv) in MeCN (20 mL) was added K₂CO₃ (3.87 g, 27.98 mmol, 2.0 equiv). The mixture was stirred at 80° C. for 12 h. The reaction mixture then cooled to room temperature and was filtered. The filtrate was concentrated under reduced pressure and the crude product was purified by silica gel chromatography (1/0 to 1/1 petroleum ether/EtOAc) to give the product (4.7 g, 71.3% yield) as a white solid.

### Step 2: Synthesis of 2-(4-(5-(((tert-butoxycarbonyl)(methyl)amino)methyl) pyrimidin-2-yl)piperazin-1-yl)pyrimidine-5-carboxylic acid

To a solution of ethyl 2-(4-(5-(((tert-butoxycarbonyl)(methyl)amino)methyl)pyrimidin-2-yl)piperazin-1-yl)pyrimidine-5-carboxylate (6 g, 13.11 mmol, 1.0 equiv) in THF (100 mL), EtOH (30 mL), and H₂O (30 mL) was added LiOH·H₂O (1.10 g, 26.23 mmol, 2.0 equiv). The mixture was stirred at room temperature for 16 h. The mixture was then concentrated under reduced pressure to remove THF and EtOH and then neutralized by the addition of 1N HCl. The resulting precipitate was collected by filtration to give the product (5.11 g, 90.1% yield) as a white solid. LCMS (ESI) m/z: [M+H] calcd for C₂₀H₂₇N₇O₄: 430.22; found 430.2.

### Building Block G. tert-butyl N-tert-butoxycarbonyl-N-((2-(2-((tert-butyl(diphenyl)silyl)oxymethyl)piperazin-1-yl)pyrimidin-5-yl)methyl)carbamate

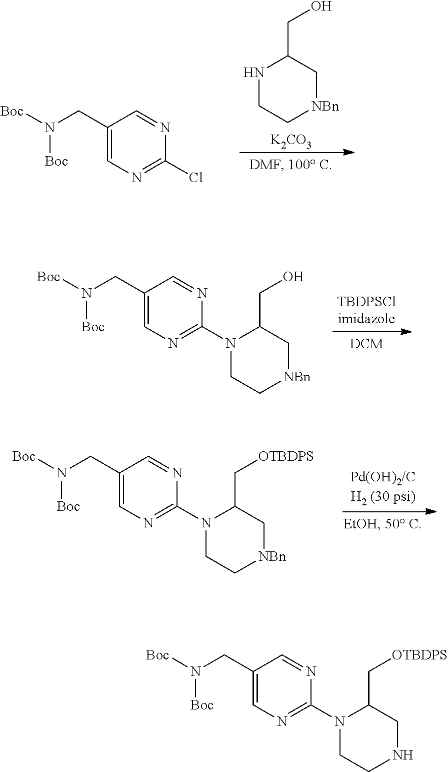

### Step 1: Synthesis of tert-butyl N-((2-(4-benzyl-2-(hydroxymethyl)piperazin-1-yl)pyrimidin-5-yl)methyl)-N-tert-butoxycarbonyl-carbamate

To a solution of tert-butyl N-tert-butoxycarbonyl-N-((2-chloropyrimidin-5-yl)methyl)carbamate (18.33 g, 53.32 mmol, 1.1 equiv) and (4-benzylpiperazin-2-yl)methanol (10 g, 48.48 mmol, 1.0 equiv) in DMF (100 mL) was added K₂CO₃ (13.40 g, 96.95 mmol, 2.0 equiv). The mixture was stirred at 100° C. for 12 h. The reaction mixture was then cooled to room temperature and H₂O (100 mL) was added. The aqueous layer was extracted with EtOAc (2×150 mL) and the combined organic layer was washed with brine (20 mL), dried with Na₂SO₄, filtered and the filtrate was concentrated under reduced pressure to give the product (7.3 g, 29.3% yield) as a yellow oil. LCMS (ESI) m/z: [M+H] calcd for C₂₇H₃₉N₅O₅: 514.31; found 514.5.

### Step 2: Synthesis of tert-butyl N-((2-(4-benzyl-2-((tert-butyl(diphenyl)silyl)oxymethyl)piperazin-1-yl)pyrimidin-5-yl)methyl)-N-tert-butoxycarbonyl-carbamate

To a solution of tert-butyl N-((2-(4-benzyl-2-(hydroxymethyl)piperazin-1-yl)pyrimidin-5-yl)methyl)-N-tert-butoxycarbonyl-carbamate (2.3 g, 4.48 mmol, 1.0 equiv) in DCM (30 mL) was added imidazole (609.69 mg, 8.96 mmol, 2.0 equiv) and TBDPSCl (1.73 mL, 6.72 mmol, 1.5 equiv). The reaction mixture was stirred at room temperature for 2 h. The mixture was then washed with H₂O (100 mL) and the aqueous phase extracted with EtOAc (2×60 mL). The combined organic phase was washed with brine (20 mL), dried with Na₂SO₄, filtered and concentrated under reduced pressure. The residue was purified by silica gel chromatography (20/1 to 3/1 petroleum ether/EtOAc) to give the product (4 g, 59.4% yield) as a yellow oil. LCMS (ESI) m/z: [M+H] calcd for C₄₃H₅₇N₅O₅Si: 752.42; found 752.4.

### Step 3: Synthesis of tert-butyl N-tert-butoxycarbonyl-N-((2-(2-((tert-butyl(diphenyl)silyl)oxymethyl)piperazin-1-yl)pyrimidin-5-yl)methyl)carbamate

To a solution of tert-butyl N-((2-(4-benzyl-2-((tert-butyl(diphenyl)silyl)oxymethyl)piperazin-1-yl)pyrimidin-5-yl)methyl)-N-tert-butoxycarbonyl-carbamate (3.3 g, 4.39 mmol, 1.0 equiv) in EtOH (10 mL) was added Pd(OH)₂/C (1 g, 10 wt. %). The mixture was heated to 50° C. under H₂ (30 psi) for 30 h. The mixture was then cooled to room temperature, filtered through Celite, and concentrated under reduced pressure. The residue was purified by silica gel chromatography (20/1 to 3/1 EtOAc/EtOH) to give the product (1.44 g, 45.6% yield) as a yellow solid. LCMS (ESI) m/z: [M+H] calcd for C₃₆H₅₁N₅O₅Si: 662.38; found 662.3.

### Building Block H. 2-(4-(5-(((tert-butoxycarbonyl)amino)methyl)pyrimidin-2-yl)-3-(hydroxymethyl)piperazin-1-yl)pyrimidine-5-carboxylic acid

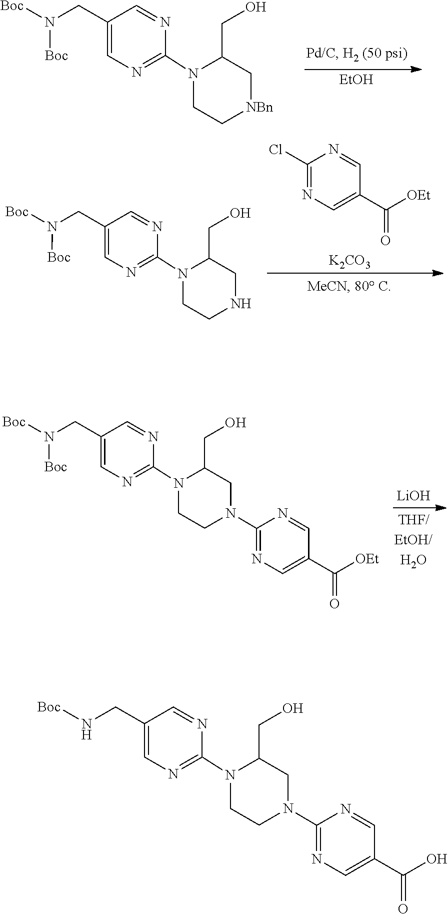

### Step 1: Synthesis of tert-butyl N-tert-butoxycarbonyl-N-((2-(2-(hydroxymethyl)piperazin-1-yl) pyrimidin-5-yl)methyl)carbamate

To a solution of tert-butyl N-((2-(4-benzyl-2-(hydroxymethyl)piperazin-1-yl)pyrimidin-5-yl)methyl)-N-tert-butoxycarbonyl-carbamate (3 g, 5.84 mmol, 1.0 equiv) in EtOH (40 mL) was added Pd/C (2 g, 10 wt. %). The suspension was degassed and purged with H₂, then stirred under H₂ (50 psi) at 30° C. for 16 h. The reaction mixture was cooled to room temperature and filtered through Celite and then concentrated under reduced pressure to give the product (1.6 g, crude) as a yellow oil. LCMS (ESI) m/z: [M+H] calcd for C₂₀H₃₃N₅O₅: 424.26; found 424.3.

### Step 2: Synthesis of ethyl 2-(4-(5-((bis(tert-butoxycarbonyl)amino)methyl)pyrimidin-2-yl)-3-(hydroxymethyl)piperazin-1-yl)pyrimidine-5-carboxylate

To a solution of tert-butyl N-tert-butoxycarbonyl-N-((2-(2-(hydroxymethyl)piperazin-1-yl)pyrimidin-5-yl)methyl)carbamate (1.4 g, 3.31 mmol, 1.0 equiv) in MeCN (20 mL) was added K₂CO₃ (2.28 g, 16.53 mmol, 5.0 equiv) and ethyl 2-chloropyrimidine-5-carboxylate (616.84 mg, 3.31 mmol, 1.0 equiv). The solution was stirred at 80° C. for 4 h. The mixture was cooled to room temperature and poured into H₂O (30 mL). The aqueous layer was extracted with EtOAc (2×30 mL) and the combined organic layer was washed with brine (20 mL), dried with Na₂SO₄, filtered and concentrated under reduced pressure. The mixture was purified by silica gel chromatography (20/1 to 3/1 petroleum ether/EtOAc) to give the product (1.6 g, 66.7% yield) as a light yellow solid. LCMS (ESI) m/z: [M+H] calcd for C₂₇H₃₉N₇O₇: 574.30; found 574.4.

### Step 3: Synthesis of 2-(4-(5-(((tert-butoxycarbonyl)amino)methyl)pyrimidin-2-yl)-3-(hydroxymethyl)piperazin-1-yl)pyrimidine-5-carboxylic acid

To a solution of ethyl 2-(4-(5-((bis(tert-butoxycarbonyl)amino)methyl)pyrimidin-2-yl)-3-(hydroxymethyl)piperazin-1-yl)pyrimidine-5-carboxylate (1.4 g, 2.44 mmol, 1.0 equiv) in THF (6 mL) and EtOH (6 mL) at 0° C. was added a solution of LiOH·H₂O (512.07 mg, 12.20 mmol, 5.0 equiv) in H₂O (3 mL). The reaction mixture was warmed to room temperature and stirred for 2 h. The mixture was then concentrated under reduced pressure to remove THF and EtOH. The aqueous phase was adjusted to pH 3 with 0.1 M HCl and the resulting suspension was filtered. The solid cake was dried under reduced pressure to give the product (613.14 mg, 55.6% yield) as a white solid. LCMS (ESI) m/z: [M+H] calcd for C₂₀H₂₇N₇O₅: 446.22; found 446.2.

### Building Block I. tert-butyl N-[(tert-butoxy)carbonyl]-N-({2-[(3R)-3-(hydroxymethyl)piperazin-1-yl]pyrimidin-5-yl}methyl)carbamate

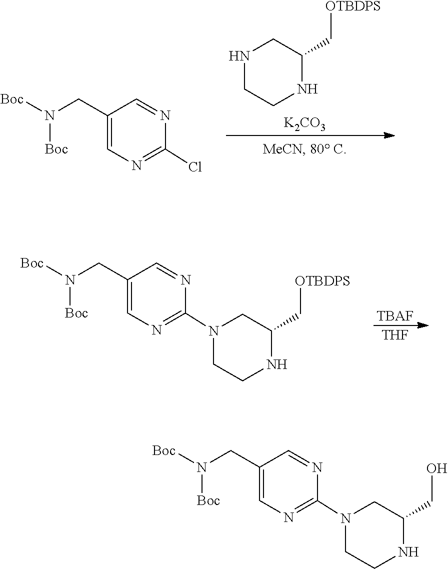

### Step 1: Synthesis of (R)-tert-butyl-N-tert-butoxycarbonyl-((2-(3-(((tert-butyldiphenylsilyl)-oxy)methyl)piperazin-1-yl)pyrimidin-5-yl)methyl)carbamate

To a solution of tert-butyl-N-tert-butoxycarbonyl-((2-chloropyrimidin-5-yl)methyl)carbamate (24.24 g, 70.51 mmol, 1.0 equiv) in MeCN (300 mL) was added (R)-2-(((tert-butyldiphenylsilyl)oxy)methyl)piperazine (25 g, 70.51 mmol, 1.0 equiv) and K₂CO₃ (29.24 g, 211.53 mmol, 3.0 equiv). The mixture was stirred at 80° C. for 16 h. The reaction mixture was then cooled to room temperature, diluted with EtOAc (200 mL), filtered and the filtrate was concentrated under reduced pressure. Purification by silica gel chromatography (0→100% EtOAc/petroleum ether) afforded the desired product (46.5 g, 94% yield) as a white solid.

### Step 2: Synthesis of tert-butyl N-[(tert-butoxy)carbonyl]-N-({2-[(3R)-3-(hydroxymethyl)piperazin-1-yl]pyrimidin-5-yl}methyl)carbamate

To a solution of (R)-tert-butyl-N-tert-butoxycarbonyl-((2-(3-(((tert-butyldiphenylsilyl)oxy)methyl)piperazin-1-yl)pyrimidin-5-yl)methyl)carbamate (12 g, 18.13 mmol, 1.0 equiv) in THF (120 mL) was added TBAF (1 M, 23.93 mL, 1.3 equiv). The mixture was stirred at room temperature for 2 h. The reaction mixture was then poured into H₂O (300 mL) and the aqueous phase was extracted with EtOAc (3×80 mL). The combined organic phases were combined, washed with brine (80 mL), dried, filtered and the filtrate was concentrated under reduced pressure. Purification by silica gel chromatography (0→20% MeOH/DCM) afforded the desired product (5 g, 64% yield) as a yellow solid.

### Building Block J. 2-{4-[5-({[(tert-butoxy)carbonyl]amino}methyl)pyrimidin-2-yl]piperazin-1-yl}pyrimidine-5-carboxylic acid

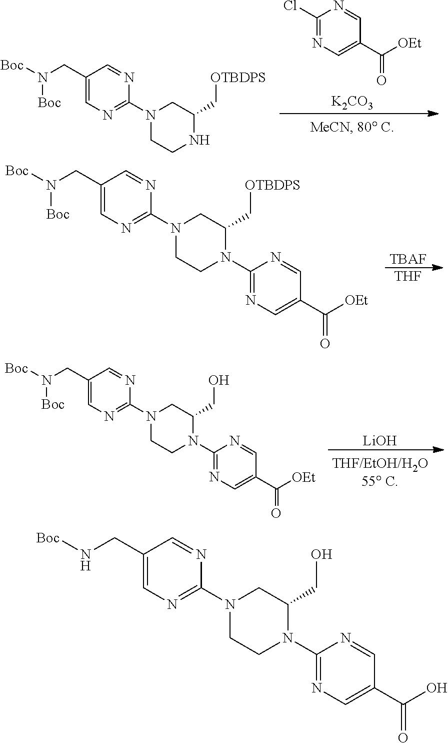

### Step 1: Synthesis of (R)-ethyl 2-(4-(5-(((di-tert-butoxycarbonyl)amino)methyl)pyrimidin-2-yl)-2-(((tert-butyldiphenylsilyl)oxy)methyl)piperazin-1-yl)pyrimidine-5-carboxylate

To a solution of (R)-tert-butyl-N-tert-butoxycarbonyl-N-((2-(3-(((tert-butyldiphenylsilyl)oxy)methyl)piperazin-1-yl)pyrimidin-5-yl)methyl)carbamate (31.5 g, 45.21 mmol, 1.0 equiv) in MeCN (350 mL) was added ethyl 2-chloropyrimidine-5-carboxylate (8.44 g, 45.21 mmol, 1.0 equiv) and K₂CO₃ (18.75 g, 135.63 mmol, 3.0 equiv). The mixture was stirred at 80° C. for 16 h. The reaction mixture was then cooled to room temperature, diluted with EtOAc (150 mL), and filtered to remove inorganic salts. The filtrate was then concentrated under reduced pressure. Purification by silica gel chromatography (0→100% EtOAc/petroleum ether) afforded the desired product (33.5 g, 89% yield).

### Step 2: Synthesis of (R)-ethyl 2-(4-(5-(((di-tert-butoxycarbonyl)amino)methyl)pyrimidin-2-yl)-2-(hydroxymethyl)piperazin-1-yl)pyrimidine-5-carboxylate

To a solution of (R)-ethyl 2-(4-(5-(((di-tert-butoxycarbonyl)amino)methyl)pyrimidin-2-yl)-2-(((tert-butyldiphenylsilyl)oxy)methyl)piperazin-1-yl)pyrimidine-5-carboxylate (36.5 g, 44.95 mmol, 1.0 equiv) in THF (300 mL) was added TBAF (1 M, 59.33 mL, 1.32 equiv). The mixture was stirred at room temperature for 6 h, at which point the reaction mixture was poured into H₂O (500 mL). The aqueous phase was separated and extracted with EtOAc (3×150 mL) and the combined organic layers were washed with brine (150 mL), dried, filtered, and the filtrate was concentrated under reduced pressure. Purification by silica gel chromatography (0→100% EtOAc/petroleum ether) afforded the desired product (17 g, 64% yield) as a yellow oil.

### Step 3: Synthesis of (R)-2-(4-(5-(((tert-butoxycarbonyl)amino)methyl)pyrimidin-2-yl)-2-(hydroxymethyl)piperazin-1-yl)pyrimidine-5-carboxylic acid

To a solution of (R)-ethyl 2-(4-(5-(((di-tert-butoxycarbonyl)amino)methyl)pyrimidin-2-yl)-2-(hydroxymethyl)piperazin-1-yl)pyrimidine-5-carboxylate (17 g, 29.64 mmol, 1.0 equiv) in H₂O (160 mL), EtOH (80 mL) and THF (160 mL) was added LiOH·H₂O (4.97 g, 118.54 mmol, 4.0 equiv). The reaction mixture was stirred at 55° C. for 16 h. To the mixture was then added LiOH·H₂O (1.01 g, 24.00 mmol, 0.81 equiv) and the reaction mixture was stirred at 55° C. for an additional 9 h. The mixture was cooled to room temperature, diluted with H₂O (150 mL), and concentrated under reduced pressure to remove THF and EtOH. The mixture was acidified (pH=5) with 1 N HCl, filtered, and the filter cake washed with H₂O (2×30 mL). The filter cake was dried under reduced pressure to afford the desired product (9.2 g, 67% yield) as a white solid. LCMS (ESI) m/z: [M+H] calcd for C₂₀H₂₇N₇O₅: 446.22; found 446.1.

### Building Block K. tert-butyl N-[(tert-butoxy)carbonyl]-N-({2-[(3S)-3-(hydroxymethyl)piperazin-1-yl]pyrimidin-5-yl}methyl)carbamate

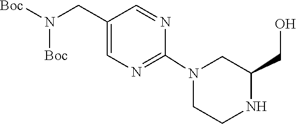 [SMILES: CC(C)(C)OC(=O)N(Cc1cnc(N2CCN[C@H](CO)C2)nc1)C(=O)OC(C)(C)C]

This building block is prepared by a process similar to that for Building block I by utilizing [(2S)-piperazin-2-yl]methanol.

### Building Block L. 2-[(2S)-4-[5-({[(tert-butoxy)carbonyl]amino}methyl)pyrimidin-2-yl]-2-(hydroxymethyl)piperazin-1-yl]pyrimidine-5-carboxylic acid

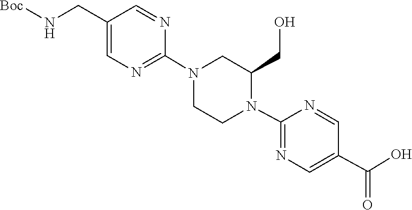 [SMILES: CC(C)(C)OC(=O)NCc1cnc(N2CCN(c3ncc(C(=O)O)cn3)[C@H](CO)C2)nc1]

This building block is prepared from Building block K by a process similar to that for Building block J.

### Building Block M. tert-butyl 2-[(3R)-3-(hydroxymethyl)piperazin-1-yl]-5H,6H,7H,8H-pyrido[4,3-d]pyrimidine-6-carboxylate

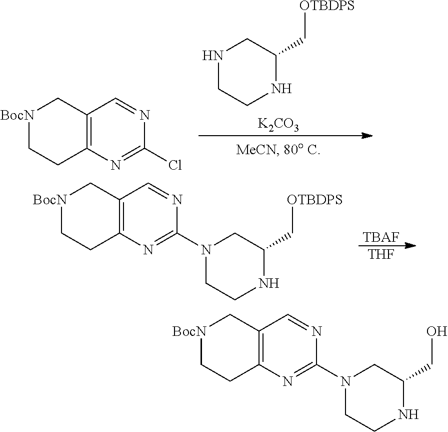

### Step 1: Synthesis of (R)-tert-butyl 2-(3-(((tert-butyldiphenylsilyl)oxy)-methyl)piperazin-1-yl)-7,8-dihydropyrido[4,3-d]pyrimidine-6(5H)-carboxylate

To a solution of (R)-2-(((tert-butyldiphenylsilyl)oxy)methyl)piperazine (25 g, 70.51 mmol, 1.0 equiv) in MeCN (250 mL) was added K₂CO₃ (29.24 g, 211.53 mmol, 3.0 equiv) and tert-butyl 2-chloro-7,8-dihydropyrido[4,3-d]pyrimidine-6(5H)-carboxylate (17.12 g, 63.46 mmol, 0.9 equiv). The mixture was stirred at 80° C. for 17 h. The reaction mixture was then cooled to room temperature, filtered, and the filtrated was concentrated under reduced pressure. Purification by silica gel chromatography (0→100% EtOAc/petroleum ether) afforded the desired product (31 g, 73.5% yield) as a white solid. LCMS (ESI) m/z: [M+H] calcd for C₃₃H₄₅N₅O₃Si: 588.34; found 588.2.

### Step 2: Synthesis of (R)-tert-butyl 2-(3-(hydroxymethyl)piperazin-1-yl)-7,8-dihydropyrido[4,3-d]pyrimidine-6(5H)-carboxylate

To a mixture of (R)-tert-butyl 2-(3-(((tert-butyldiphenylsilyl)oxy)methyl)piperazin-1-yl)-7,8-dihydropyrido[4,3-d]pyrimidine-6(5H)-carboxylate (12 g, 20.41 mmol, 1.0 equiv) in THF (120 mL) was added TBAF (1.0 M, 24.50 mL, 1.2 equiv). The mixture was stirred at room temperature for 5 h. The mixture was poured into H₂O (100 mL), and the aqueous phase was extracted with EtOAc (2×100 mL). The combined organic phases were washed with brine (100 mL), dried, filtered, and the filtrate was concentrated under reduced pressure. Purification by silica gel chromatography (0→10% MeOH/DCM) afforded the desired product (6 g, 84.1% yield) as a white solid. LCMS (ESI) m/z: [M+H] calcd for C₁₇H₂₇N₅O₃: 350.22; found 350.2.

### Building Block N. 2-[(2R)-4-{6-[(tert-butoxy)carbonyl]-5H,6H,7H,8H-pyrido[4,3-d]pyrimidin-2-yl}-2-(hydroxymethyl)piperazin-1-yl]pyrimidine-5-carboxylic acid

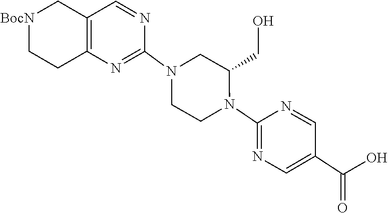 [SMILES: CC(C)(C)OC(=O)N1CCc2nc(N3CCN(c4ncc(C(=O)O)cn4)[C@@H](CO)C3)ncc2C1]

This building block is prepared from Building block M by a process similar to that for Building block J.

### Building Block O. tert-butyl 2-[(3S)-3-(hydroxymethyl)piperazin-1-yl]-5H,6H,7H,8H-pyrido[4,3-d]pyrimidine-6-carboxylate

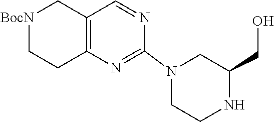 [SMILES: CC(C)(C)OC(=O)N1CCc2nc(N3CCN[C@H](CO)C3)ncc2C1]

This building block is prepared by a process similar to that for Building block I by utilizing tert-butyl 2-chloro-7,8-dihydropyrido[4,3-d]pyrimidine-6(5H)-carboxylate and [(2S)-piperazin-2-yl]methanol.

### Building Block P. 2-[(2S)-4-{6-[(tert-butoxy)carbonyl]-5H,6H,7H,8H-pyrido[4,3-d]pyrimidin-2-yl}-2-(hydroxymethyl)piperazin-1-yl]pyrimidine-5-carboxylic acid

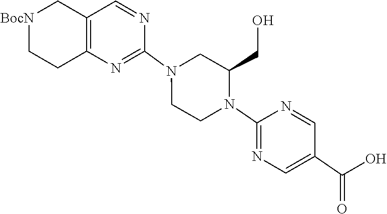 [SMILES: CC(C)(C)OC(=O)N1CCc2nc(N3CCN(c4ncc(C(=O)O)cn4)[C@H](CO)C3)ncc2C1]

This building block is prepared from Building block O by a process similar to that for Building block J.

### Building Block Q. tert-butyl N-[(tert-butoxy)carbonyl]-N-({2-[(3S)-3-[(dimethylamino)methyl]piperazin-1-yl]pyrimidin-5-yl}methyl)carbamate

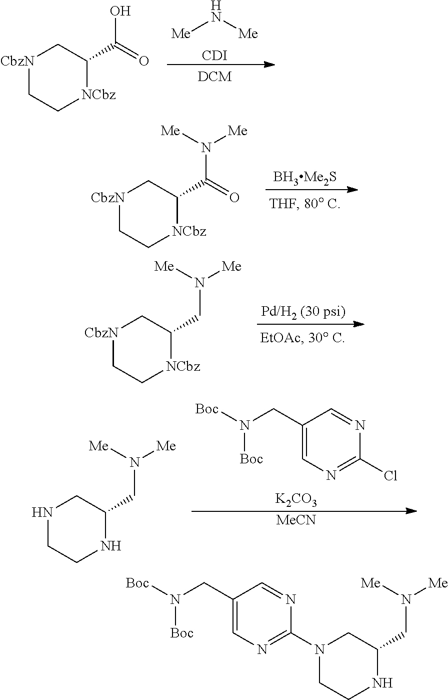

### Step 1: Synthesis of (R)-dibenzyl 2-(dimethylcarbamoyl)piperazine-1,4-dicarboxylate

To a solution of CDI (12.21 g, 75.30 mmol, 1.2 equiv) in DCM (300 mL) at 0° C. was added (R)-1,4-bis((benzyloxy)carbonyl)piperazine-2-carboxylic acid (25 g, 62.75 mmol, 1.0 equiv). The mixture was stirred at 0° C. for 0.5 h, at which time dimethylamine (8.51 mL, 92.87 mmol, 1.5 equiv, HCl) was added. The reaction mixture was warmed to room temperature and stirred for 12 h. The reaction mixture was then added to H₂O (200 mL), and the aqueous layer was separated and extracted with DCM (2×200 mL). The combined organic phases were washed with brine (2×50 mL), dried, filtered, and the filtrate was concentrated under reduced pressure. Purification by silica gel chromatography (50→100% EtOAc/petroleum ether) afforded the desired product (23.5 g, 88.0% yield) as a yellow oil.

### Step 2: Synthesis of (S)-dibenzyl 2-((dimethylamino)methyl)piperazine-1,4-dicarboxylate

To a solution of (R)-dibenzyl 2-(dimethylcarbamoyl)piperazine-1,4-dicarboxylate (28 g, 65.81 mmol, 1.0 equiv) in THF (300 mL) at 0° C. was added BH₃·Me₂S (10 M, 13.16 mL, 2.0 equiv). The reaction mixture was then stirred at 80° C. for 3 h. The reaction mixture was cooled to room temperature and then MeOH (50 mL) was added. After stirring for an additional 1 h the mixture was concentrated under reduced pressure. Purification by silica gel chromatography (50→100% EtOAc/petroleum ether) afforded the desired product (18 g, 66.5% yield) as a yellow oil.

### Step 3: Synthesis of (R)—N,N-dimethyl-1-(piperazin-2-yl)methanamine

To a solution of (S)-dibenzyl 2-((dimethylamino)methyl)piperazine-1,4-dicarboxylate (18 g, 43.74 mmol, 1.0 equiv) in EtOAc (200 mL) was added Pd/C (1.5 g, 10 wt. %). The suspension was degassed under reduced pressure and purged with H₂ three times. The suspension was stirred under H₂(30 psi) at 30° C. for 5 h. The reaction mixture was then filtered through celite and the filtrate was concentrated under reduced pressure to afford the desired product (6 g, 95.8% yield) as a yellow solid.

### Step 4: Synthesis of tert-butyl N-tert-butoxycarbonyl-N-((2-((3S)-3-((dimethylamino)methyl)piperazin-1-yl)pyrimidin-5-yl)methyl)carbamate

To a solution of (R)—N,N-dimethyl-1-(piperazin-2-yl)methanamine (2.8 g, 19.55 mmol, 1.0 equiv) in MeCN (40 mL) was added tert-butyl N-tert-butoxycarbonyl-N-((2-chloropyrimidin-5-yl)methyl)carbamate (6.72 g, 19.55 mmol, 1.0 equiv) and K₂CO₃ (5.40 g, 39.10 mmol, 2.0 equiv). The mixture was stirred at 80° C. for 24 h. The mixture was then cooled to room temperature, filtered, and the filter cake washed with EtOAc (3×10 mL). The filtrate was then concentrated under reduced pressure. Purification by silica gel chromatography (0→100% MeOH/EtOAc) afforded the desired product (5.3 g, 57.8% yield) as a yellow oil. LCMS (ESI) m/z: [M+H] calcd for C₂₂H₃₈N₆O₄: 451.31; found 451.2.

### Building Block R. 2-[(2S)-4-[5-({[(tert-butoxy)carbonyl]amino}methyl)pyrimidin-2-yl]-2-[(dimethylamino)methyl]piperazin-1-yl]pyrimidine-5-carboxylic acid

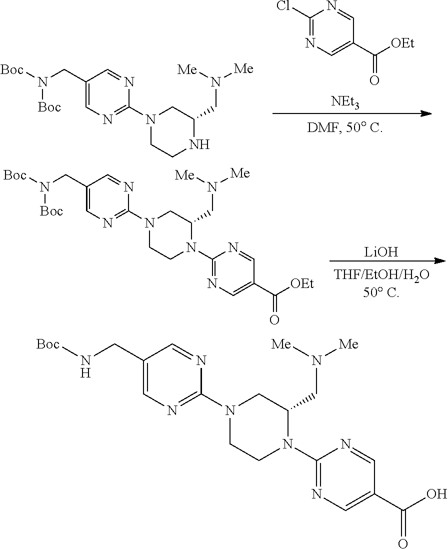

### Step 1: Synthesis of (S)-ethyl 2-(4-(5-(((bi-tert-butoxycarbonyl)amino)methyl) pyrimidin-2-yl)-2-((dimethylamino)methyl)piperazin-1-yl)pyrimidine-5-carboxylate

To a solution of (S)-tert-butyl-N-tert-butoxycarbonyl ((2-(3-((dimethylamino)methyl) piperazin-1-yl)pyrimidin-5-yl)methyl)carbamate (3.26 g, 7.24 mmol, 1.0 equiv) in DMF (30 mL) was added Et₃N (3.02 mL, 21.71 mmol, 3.0 equiv) and ethyl 2-chloropyrimidine-5-carboxylate (1.47 g, 7.86 mmol, 1.1 equiv). The mixture was stirred at 50° C. for 3 h and then concentrated under reduced pressure to afford the desired product (4.35 g, crude) as a solution in DMF (30 mL), which was used directly in the next step. LCMS (ESI) m/z: [M+H] calcd for C₂₉H₄₄N₈O₆: 601.35; found 601.5.

### Step 2: Synthesis of (S)-2-(4-(5-(((tert-butoxycarbonyl)amino)methyl)-pyrimidin-2-yl)-2-((dimethylamino)methyl)piperazin-1-yl)pyrimidine-5-carboxylic acid

To a solution of (S)-ethyl 2-(4-(5-(((bi-tert-butoxycarbonyl)amino)methyl)-pyrimidin-2-yl)-2-((dimethylamino)methyl)piperazin-1-yl)pyrimidine-5-carboxylate (4.35 g, 7.24 mmol, 1.0 equiv) in DMF (30 mL) was added DMF (50 mL), EtOH (30 mL), and H₂O (30 mL). To the solution was then added LiOH·H₂O (3 g, 71.50 mmol, 9.9 equiv) at 50° C. The reaction was stirred at 50° C. for 36 h. The mixture was then cooled to room temperature, neutralized with 0.5 N HCl, and concentrated under reduced pressure. Purification by reverse phase chromatography (2→30% MeCN/H₂O) afforded the desired product (1.15 g, 34% yield) as a white solid. LCMS (ESI) m/z: [M+H] calcd for C₂₂H₃₂N₈O₄: 473.26; found 473.3.

### Building Block S. tert-butyl N-[(tert-butoxy)carbonyl]-N-({2-[(3R)-3-[(dimethylamino)methyl]piperazin-1-yl]pyrimidin-5-yl}methyl)carbamate

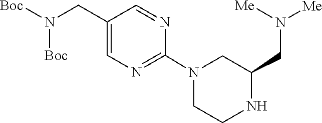 [SMILES: CN(C)C[C@@H]1CN(c2ncc(CN(C(=O)OC(C)(C)C)C(=O)OC(C)(C)C)cn2)CCN1]

This building block is prepared by a process similar to that for Building block I by utilizing dimethyl({[(2S)-piperazin-2-yl]methyl})amine.

### Building Block T. 2-[(2R)-4-[5-({[(tert-butoxy)carbonyl]amino}methyl)pyrimidin-2-yl]-2-[(dimethylamino)methyl]piperazin-1-yl]pyrimidine-5-carboxylic acid

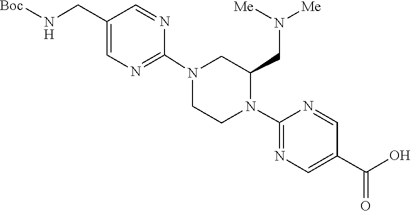 [SMILES: CN(C)C[C@@H]1CN(c2ncc(CNC(=O)OC(C)(C)C)cn2)CCN1c1ncc(C(=O)O)cn1]

This building block is prepared from Building block S by a process similar to that for Building block J.

### Building Block U. tert-butyl 2-[(3S)-3-[(dimethylamino)methyl]piperazin-1-yl]-5H,6H,7H,8H-pyrido[4,3-d]pyrimidine-6-carboxylate

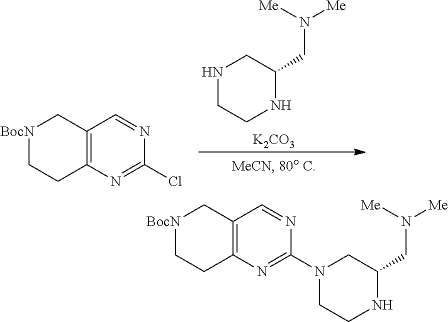

To a solution of tert-butyl 2-chloro-7,8-dihydropyrido[4,3-d]pyrimidine-6(5H)-carboxylate (4.80 g, 17.80 mmol, 1.4 equiv) in MeCN (45 mL) was added K₂CO₃ (10.42 g, 75.40 mmol, 3.0 equiv) and (R)—N,N-dimethyl-1-(piperazin-2-yl)methanamine (3.6 g, 25.13 mmol, 1.0 equiv). The mixture was stirred at 80° C. for 8 h. The mixture was then cooled to room temperature, filtered, and the filter cake was washed with EtOAc (50 mL). To the organic phase was added H₂O (50 mL) and the aqueous phase was extracted with EtOAc (3×50 mL). The combined organic phases were washed with brine (5 mL), dried, filtered and concentrated under reduced pressure. Purification by silica gel chromatography (8→67% EtOAc/petroleum ether) afforded the desired product (6.5 g, 63.5% yield) as a yellow oil.

### Building Block V. 2-[(2S)-4-{6-[(tert-butoxy)carbonyl]-5H,6H,7H,8H-pyrido[4,3-d]pyrimidin-2-yl}-2-[(dimethylamino)methyl]piperazin-1-yl]pyrimidine-5-carboxylic

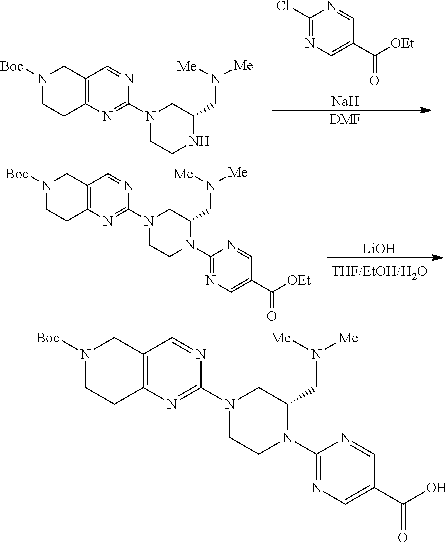

### Step 1: Synthesis of (S)-tert-butyl 2-(3-((dimethylamino)methyl)-4-(5-(ethoxycarbonyl)pyrimidin-2-yl)piperazin-1-yl)-7,8-dihydropyrido[4,3-d]pyrimidine-6(5H)-carboxylate

To a solution of (S)-tert-butyl 2-(3-((dimethylamino)methyl)piperazin-1-yl)-7,8-dihydropyrido[4,3-d]pyrimidine-6(5H)-carboxylate (3 g, 7.97 mmol, 1.0 equiv) in DMF (70 mL) at 0° C. was added NaH (382.44 mg, 9.56 mmol, 60 wt. %, 1.2 equiv). The suspension was stirred at 0° C. for 0.5 h, then ethyl 2-chloropyrimidine-5-carboxylate (1.49 g, 7.97 mmol, 1 equiv) in DMF (50 mL) was added, dropwise. The mixture was warmed to room temperature and stirred for 5 h. The mixture was then cooled to 0° C. and poured into H₂O (360 mL). The suspension was filtered, and the filter cake washed with H₂O (30 mL) and dried under reduced pressure. Purification by silica gel chromatography (6%→33% EtOAc/petroleum ether) afforded the desired product (1.8 g, 39.6% yield) as a brown oil.

### Step 2: Synthesis of (S)-2-(4-(6-(tert-butoxycarbonyl)-5,6,7,8-tetrahydropyrido[4,3-d]pyrimidin-2-yl)-2-((dimethylamino)methyl)piperazin-1-yl)pyrimidine-5-carboxylic acid

To a solution of (S)-tert-butyl 2-(3-((dimethylamino)methyl)-4-(5-(ethoxycarbonyl)pyrimidin-2-yl)piperazin-1-yl)-7,8-dihydropyrido[4,3-d]pyrimidine-6(5H)-carboxylate (1.1 g, 2.09 mmol, 1.0 equiv) in THF (5 mL), EtOH (2.5 mL), and H₂O (2.5 mL) was added LiOH·H₂O (175.30 mg, 4.18 mmol, 2.0 equiv). The mixture was stirred at room temperature for 2 h, at which point the pH was adjusted to 7 by the addition of 1 N HCl at 0° C. The mixture was concentrated under reduced pressure to remove THF and MeOH. The resulting suspension was filtered, and the filter cake was washed with H₂O (5 mL) and dried under reduced pressure to afford the desired product (680 mg, 65.3% yield) as a white solid. LCMS (ESI) m/z: [M+H] calcd for C₂₄H₃₄N₈O₄: 499.28; found 499.2.

### Building Block W. tert-butyl 2-[(3R)-3-[(dimethylamino)methyl]piperazin-1-yl]-5H,6H,7H,8H-pyrido[4,3-d]pyrimidine-6-carboxylate

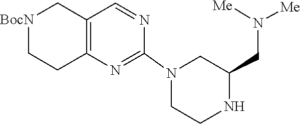 [SMILES: CN(C)C[C@@H]1CN(c2ncc3c(n2)CCN(C(=O)OC(C)(C)C)C3)CCN1]

This building block is prepared by a process similar to that for Building block I by utilizing tert-butyl 2-chloro-7,8-dihydropyrido[4,3-d]pyrimidine-6(5H)-carboxylate and dimethyl({[(2S)-piperazin-2-yl]methyl})amine.

### Building Block X. 2-[(2R)-4-{6-[(tert-butoxy)carbonyl]-5H,6H,7H,8H-pyrido[4,3-d]pyrimidin-2-yl}-2-[(dimethylamino)methyl]piperazin-1-yl]pyrimidine-5-carboxylic acid

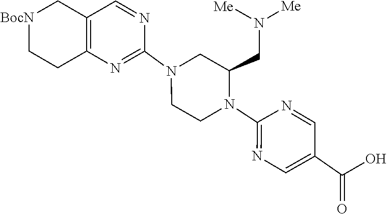 [SMILES: CN(C)C[C@@H]1CN(c2ncc3c(n2)CCN(C(=O)OC(C)(C)C)C3)CCN1c1ncc(C(=O)O)cn1]

This building block is prepared from Building block W by a process similar to that for Building block J.

### Building Block Y. tert-butyl (2R)-4-{6-[(tert-butoxy)carbonyl]-5H,6H,7H,8H-pyrido[4,3-d]pyrimidin-2-yl}piperazine-2-carboxylate

### Step 1: Synthesis of (R)-1,4-bis((benzyloxy)carbonyl)piperazine-2-carboxylic acid

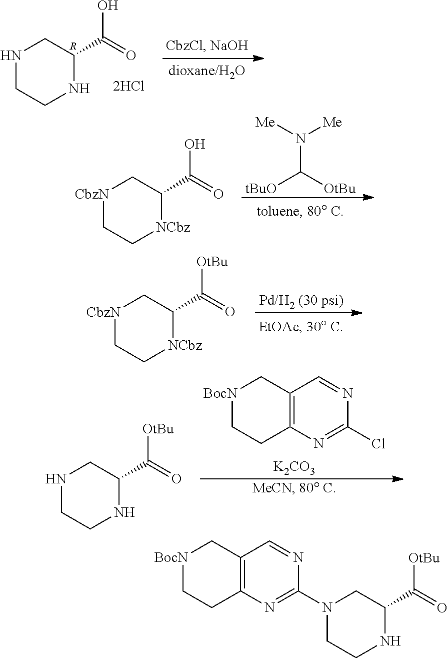

To a solution of (R)-piperazine-2-carboxylic acid (70 g, 344.71 mmol, 1.0 equiv, 2HCl) in dioxane (1120 mL) and H₂O (700 mL) was added 50% aq. NaOH until the solution was pH=11. Benzyl chloroformate (156.82 mL, 1.10 mol, 3.2 equiv) was added and the reaction mixture was stirred at room temperature for 12 h. To the solution was then added H₂O (1200 mL) and the aqueous layer was washed with MTBE (3×800 mL). The aqueous layer was adjusted to pH=2 with concentrated HCl (12N) and extracted with EtOAc (2×1000 mL). The combined organic extracts were dried, filtered and the filtrate was concentrated under reduced pressure to afford the desired product (137 g, 99.8% yield) as a yellow solid. LCMS (ESI) m/z: [M+H] calcd for C₂₁H₂₂N₂O₆: 399.16; found 399.2.

### Step 2: Synthesis of (R)-1,4-dibenzyl 2-tert-butyl piperazine-1,2,4-tricarboxylate

To a solution of (R)-1,4-bis((benzyloxy)carbonyl)piperazine-2-carboxylic acid (50 g, 125.50 mmol, 1.0 equiv) in toluene (500 mL) at 80° C. was added 1,1-di-tert-butoxy-N,N-dimethylmethanamine (57.17 mL, 238.45 mmol, 1.9 equiv). The solution was stirred at 80° C. for 2 h, at which point the reaction mixture was cooled to room temperature and partitioned between EtOAc (300 mL) and H₂O (500 mL). The aqueous layer was extracted with EtOAc (2×500 mL) and the combined organic layers were dried, filtered and the filtrate was concentrated under reduced pressure. Purification by silica gel chromatography (0%→25% EtOAc/petroleum ether) afforded the desired product (35 g, 61.2% yield) as a white solid. LCMS (ESI) m/z: [M+Na] calcd for C₂₅H₃₀N₂O₆: 477.20; found 477.1.

### Step 3: Synthesis of (R)-tert-butyl piperazine-2-carboxylate

To a solution of (R)-1,4-dibenzyl 2-tert-butyl piperazine-1,2,4-tricarboxylate (35 g, 77.01 mmol, 1.0 equiv) in EtOAc (350 mL) was added Pd/C (10 g, 10 wt. %). The suspension was degassed under reduced pressure and purged with H₂ three times. The mixture was stirred under H₂ (30 psi) at 30° C. for 4 h. The reaction mixture was then filtered through celite, the residue was washed with MeOH (5×200 mL), and the filtrate was concentrated under reduced pressure to afford the desired product (14 g, 79.6% yield) as yellow oil. LCMS (ESI) m/z: [M+H] calcd for C₉H₁₈N₂O₂: 187.15; found 187.1.

### Step 4: Synthesis of (R)-tert-butyl 2-(3-(tert-butoxycarbonyl)piperazin-1-yl)-7,8-dihydropyrido[4,3-d]pyrimidine-6(5H)-carboxylate

To a solution of tert-butyl (2R)-piperazine-2-carboxylate (12 g, 64.43 mmol, 1.0 equiv) in MeCN (200 mL) was added K₂CO₃ (17.81 g, 128.86 mmol, 2.0 equiv) and tert-butyl 2-chloro-7,8-dihydropyrido[4,3-d]pyrimidine-6(5H)-carboxylate (17.38 g, 64.43 mmol, 1.0 equiv). The reaction mixture was heated to 80° C. and stirred for 12 h. The reaction mixture was then cooled to room temperature and filtered, the residue was washed with EtOAc (3×150 mL), and the filtrate was concentrated under reduced pressure. Purification by silica gel chromatography (0%→100% EtOAc/petroleum ether) afforded the desired product (19 g, 69.2% yield) as a yellow solid. LCMS (ESI) m/z: [M+H] calcd for C₂₁H₃₃N₅O₄: 420.26; found 420.2.

### Building Block Z. 4-amino-2-[(2R)-2-[(tert-butoxy)carbonyl]-4-{6-[(tert-butoxy)carbonyl]-5H,6H,7H,8H-pyrido[4,3-d]pyrimidin-2-yl}piperazin-1-yl]pyrimidine-5-carboxylic acid

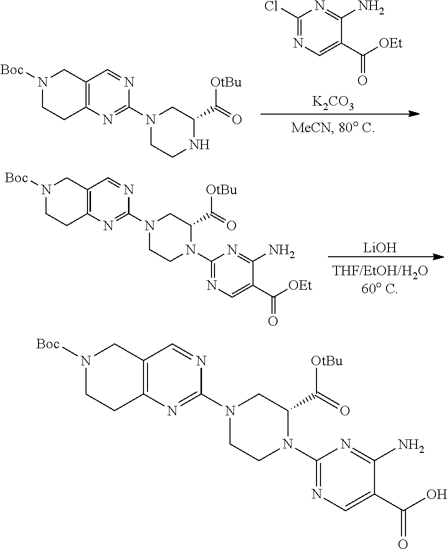 [SMILES: CC(C)(C)OC(=O)[C@H]1CN(c2ncc3c(n2)CCN(C(=O)OC(C)(C)C)C3)CCN1 ; "K2CO3" ; CCOC(=O)c1cnc(Cl)nc1N ; CCOC(=O)c1cnc(N2CCN(c3ncc4c(n3)CCN(C(=O)OC(C)(C)C)C4)C[C@@H]2C(=O)OC(C)(C)C)nc1N ; "LiOH" ; CC(C)(C)OC(=O)[C@H]1CN(c2ncc3c(n2)CCN(C(=O)OC(C)(C)C)C3)CCN1c1ncc(C(=O)O)c(N)n1]

### Step 1: Synthesis of (R)-tert-butyl 2-(4-(4-amino-5-(ethoxycarbonyl)pyrimidin-2-yl)-3-(tert-butoxycarbonyl)piperazin-1-yl)-7,8-dihydropyrido[4,3-d]pyrimidine-6(5H)-carboxylate

To a stirred solution of (R)-tert-butyl 2-(3-(tert-butoxycarbonyl)piperazin-1-yl)-7,8-dihydropyrido[4,3-d]pyrimidine-6(5H)-carboxylate (12 g, 28.60 mmol, 1.0 equiv) in MeCN (150 mL) was added K₂CO₃ (7.91 g, 57.20 mmol, 2.0 equiv) and ethyl 4-amino-2-chloropyrimidine-5-carboxylate (6.92 g, 34.32 mmol, 1.2 equiv). The reaction mixture was stirred at 80° C. for 12 h, at which point the reaction mixture was filtered and the filtrate was concentrated under reduced pressure. Purification by silica gel chromatography (0%→17% EtOAc/petroleum ether) afforded the desired product (16 g, 91.6% yield) as a yellow solid. LCMS (ESI) m/z: [M+H] calcd for C₂₈H₄₀N₈O₆: 585.32; found 585.1.

### Step 2: Synthesis of (R)-4-amino-2-(2-(tert-butoxycarbonyl)-4-(6-(tert-butoxycarbonyl)-5,6,7,8-tetrahydropyrido[4,3-d]pyrimidin-2-yl)piperazin-1-yl)pyrimidine-5-carboxylic acid

To two separate batches run in parallel each containing a solution of (R)-tert-butyl 2-(4-(4-amino-5-(ethoxycarbonyl)pyrimidin-2-yl)-3-(tert-butoxycarbonyl)piperazin-1-yl)-7,8-dihydropyrido[4,3-d]pyrimidine-6(5H)-carboxylate (7 g, 11.97 mmol, 1.0 equiv) in THF (70 mL), EtOH (35 mL) and H₂O (35 mL) was added LiOH·H₂O (2.01 g, 47.89 mmol, 4.0 equiv). The mixtures were stirred at 60° C. for 3 h, at which point the two reaction mixtures were combined, and were adjusted to pH=7 with 1 N HCl. The mixture was concentrated under reduced pressure to remove THF and EtOH, filtered, and the residue was dried under reduced pressure. The residue was stirred in MTBE (100 mL) for 10 min, filtered, and the residue was dried under reduced pressure to afford the desired product (8.02 g, 55.1% yield) as a white solid. LCMS (ESI) m/z: [M+H] calcd for C₂₆H₃₆N₈O₆:557.29; found 557.3.

### Building Block AA. tert-butyl (2S)-4-{6-[(tert-butoxy)carbonyl]-5H,6H,7H,8H-pyrido[4,3-d]pyrimidin-2-yl}piperazine-2-carboxylate

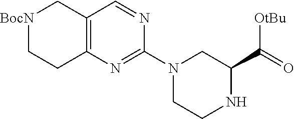 [SMILES: CC(C)(C)OC(=O)[C@@H]1CN(c2ncc3c(n2)CCN(C(=O)OC(C)(C)C)C3)CCN1]

This building block is prepared by a process similar to that for Building block I by utilizing tert-butyl 2-chloro-7,8-dihydropyrido[4,3-d]pyrimidine-6(5H)-carboxylate and tert-butyl (2S)-piperazine-2-carboxylate.

### Building Block AB. 4-amino-2-[(2S)-2-[(tert-butoxy)carbonyl]-4-{6-[(tert-butoxy)carbonyl]-5H,6H,7H,8H-pyrido[4,3-d]pyrimidin-2-yl}piperazin-1-yl]pyrimidine-5-carboxylic acid

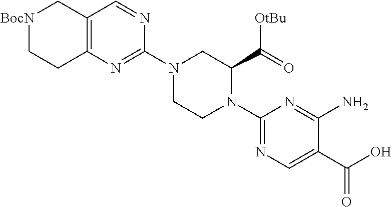 [SMILES: CC(C)(C)OC(=O)[C@@H]1CN(c2ncc3c(n2)CCN(C(=O)OC(C)(C)C)C3)CCN1c1ncc(C(=O)O)c(N)n1]

This building block is prepared from Building block AA by a process similar to that for Building block J by utilizing ethyl 4-amino-2-chloropyrimidine-5-carboxylate.

### Building Block AC. 4-amino-2-(4-{6-[(tert-butoxy)carbonyl]-5H,6H,7H,8H-pyrido[4,3-d]pyrimidin-2-yl}piperazin-1-yl)pyrimidine-5-carboxylic acid

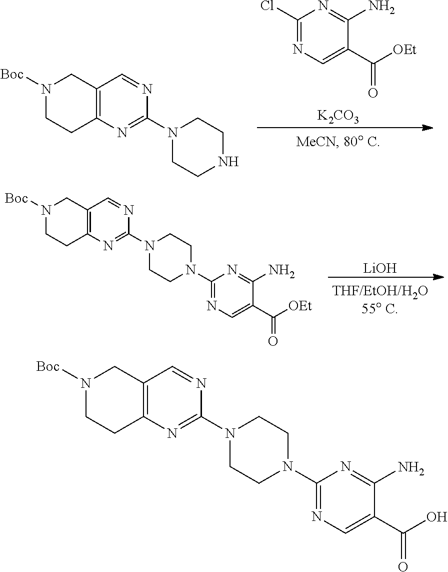 [SMILES: CC(C)(C)OC(=O)N1CCc2nc(N3CCNCC3)ncc2C1 ; "K2CO3" ; CCOC(=O)c1cnc(Cl)nc1N ; CCOC(=O)c1cnc(N2CCN(c3ncc4c(n3)CCN(C(=O)OC(C)(C)C)C4)CC2)nc1N ; "LiOH" ; CC(C)(C)OC(=O)N1CCc2nc(N3CCN(c4ncc(C(=O)O)c(N)n4)CC3)ncc2C1]

### Step 1: Synthesis of tert-butyl 2-(4-(4-amino-5-(ethoxycarbonyl)pyrimidin-2-yl)piperazin-1-yl)-7,8-dihydropyrido[4,3-d]pyrimidine-6(5H)-carboxylate

To a solution of tert-butyl 2-(piperazin-1-yl)-7,8-dihydropyrido[4,3-d]pyrimidine-6(5H)-carboxylate (8.3 g, 25.99 mmol, 1.0 equiv) and ethyl 4-amino-2-chloropyrimidine-5-carboxylate (5.24 g, 25.99 mmol, 1.0 equiv) in MeCN (100 mL) was added to K₂CO₃ (7.18 g, 51.97 mmol, 2.0 equiv). The reaction was stirred at 80° C. for 12 h. The reaction was then cooled to room temperature, DCM (100 mL) was added, and the reaction mixture was stirred for 30 min. The suspension was filtered, and the filter cake was washed with DCM (6×100 mL). The filtrate was concentrated under reduced pressure and the residue was triturated with EtOAc (30 mL), filtered and then the filter cake was dried under reduced pressure to afford the desired product (8.7 g, 65.9% yield) as light yellow solid.

### Step 2: Synthesis of 4-amino-2-(4-(6-(tert-butoxycarbonyl)-5,6,7,8-tetrahydropyrido[4,3-d]pyrimidin-2-yl)piperazin-1-yl)pyrimidine-5-carboxylic acid

To a solution of tert-butyl 2-(4-(4-amino-5-(ethoxycarbonyl)pyrimidin-2-yl)piperazin-1-yl)-7,8-dihydropyrido[4,3-d]pyrimidine-6(5H)-carboxylate (8.7 g, 17.95 mmol, 1.0 equiv) in THF (120 mL), EtOH (60 mL), and H₂O (60 mL) was added LiOH·H₂O (1.51 g, 35.91 mmol, 2.0 equiv). The mixture was stirred at 55° C. for 12 h. The reaction mixture was then concentrated under reduced pressure to remove EtOH and THF, and the reaction mixture was adjusted to pH=6 by the addition of 1 N HCl. The precipitate was filtered, and the filter cake was washed with H₂O (3×50 mL) and then dried under reduced pressure to afford the desired product (7.3 g, 89.1% yield) as light yellow solid. LCMS (ESI) m/z: [M+H] calcd for C₂₁H₂₈N₈O₄: 457.23; found 457.2.

### Building Block AD. 4-amino-2-{4-[5-({[(tert-butoxy)carbonyl]amino}methyl)pyrimidin-2-yl]piperazin-1-yl}pyrimidine-5-carboxylic acid

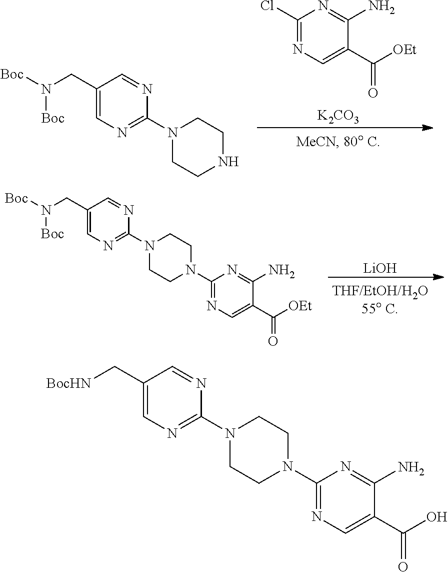 [SMILES: CC(C)(C)OC(=O)N(Cc1cnc(N2CCNCC2)nc1)C(=O)OC(C)(C)C ; "K2CO3" ; CCOC(=O)c1cnc(Cl)nc1N ; CCOC(=O)c1cnc(N2CCN(c3ncc(CN(C(=O)OC(C)(C)C)C(=O)OC(C)(C)C)cn3)CC2)nc1N ; "LiOH" ; CC(C)(C)OC(=O)NCc1cnc(N2CCN(c3ncc(C(=O)O)c(N)n3)CC2)nc1]

### Step 1: Synthesis of ethyl 4-amino-2-(4-(5-(((di-tert-butoxycarbonyl)amino)methyl)pyrimidin-2-yl)piperazin-1-yl)pyrimidine-5-carboxylate

To a solution of tert-butyl-N-tert-butoxycarbonyl-N-((2-(piperazin-1-yl)pyrimidin-5-yl)methyl)carbamate (8.3 g, 21.09 mmol, 1.0 equiv) in MeCN (100 mL) was added ethyl 4-amino-2-chloropyrimidine-5-carboxylate (4.04 g, 20.04 mmol, 0.95 equiv) and K₂CO₃ (8.75 g, 63.28 mmol, 3.0 equiv). The mixture was stirred at 80° C. for 3 h. The reaction was then cooled to room temperature, DCM (150 mL) was added, and the reaction mixture was stirred for 30 min. The suspension was filtered, the filter cake was washed with DCM (3×100 mL), and the filtrate was concentrated under reduced pressure. Purification by silica gel chromatography (0%→100% EtOAc/petroleum ether) afforded the desired product (8.35 g, 67% yield) as a white solid.

### Step 2: Synthesis of 4-amino-2-(4-(5-(((tert-butoxycarbonyl)amino)methyl)pyrimidin-2-yl)piperazin-1-yl)pyrimidine-5-carboxylic acid

To a solution of ethyl 4-amino-2-(4-(5-(((di-tert-butoxycarbonyl)amino)methyl)pyrimidin-2-yl)piperazin-1-yl)pyrimidine-5-carboxylate (8.3 g, 14.86 mmol, 1.0 equiv) in H₂O (70 mL), EtOH (36 mL) and THF (80 mL) was added LiOH·H₂O (2.49 g, 59.43 mmol, 4.0 equiv). The reaction mixture was stirred at 55° C. for 16 h. The mixture was then concentrated under reduced pressure to remove THF and EtOH. The mixture was diluted with H₂O (55 mL) and was adjusted to pH=6 by the addition of 1 N HCl. The mixture was filtered, and the filter cake was washed with H₂O (2×20 mL). The solid cake was dried under reduced pressure to afford the desired product (5.5 g, 84% yield) as a white solid. LCMS (ESI) m/z: [M+H] calcd for C₁₉H₂₆N₈O₄: 431.22; found 431.4.

### Building Block AE. 4-amino-2-[(2R)-4-{6-[(tert-butoxy)carbonyl]-5H,6H,7H,8H-pyrido[4,3-d]pyrimidin-2-yl}-2-(hydroxymethyl)piperazin-1-yl]pyrimidine-5-carboxylic acid

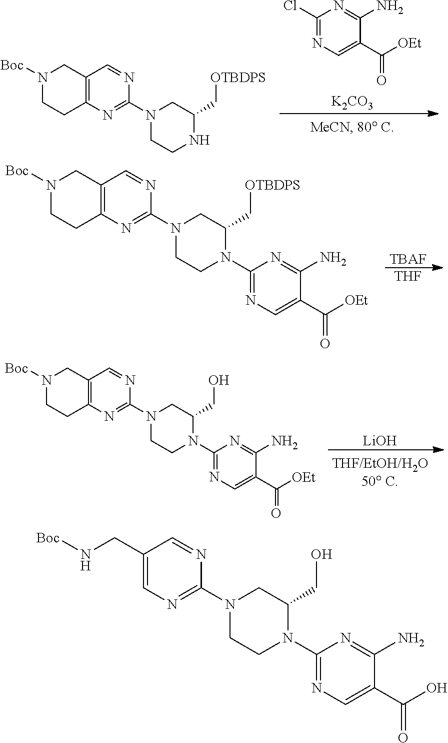

### Step 1: Synthesis of (R)-tert-butyl 2-(4-(4-amino-5-(ethoxycarbonyl)pyrimidin-2-yl)-3-(((tert-butyldiphenylsilyl)oxy)methyl)piperazin-1-yl)-7,8-dihydropyrido[4,3-d]pyrimidine-6(5H)-carboxylate

To a solution of (R)-tert-butyl 2-(3-(((tert-butyldiphenylsilyl)oxy)methyl) piperazin-1-yl)-7,8-dihydropyrido[4,3-d]pyrimidine-6(5H)-carboxylate (17.2 g, 29.26 mmol, 1.0 equiv) in MeCN (200 mL) was added K₂CO₃ (12.13 g, 87.78 mmol, 3.0 equiv) and ethyl 4-amino-2-chloropyrimidine-5-carboxylate (6.37 g, 31.60 mmol, 1.08 equiv). The mixture was stirred at 80° C. for 18 h. The reaction mixture was then cooled to room temperature, filtered and the filtrate was concentrated under reduced pressure. Purification by silica gel chromatography (0%→33% EtOAc/petroleum ether) afforded the desired product (20.3 g, 90.6% yield) as a white solid. LCMS (ESI) m/z: [M+H] calcd for C₄₀H₅₂N₈O₅Si: 753.39; found 753.4.

### Step 2: Synthesis of (R)-4-amino-2-(4-(6-(tert-butoxycarbonyl)-5,6,7,8-tetrahydropyrido[4,3-d]pyrimidin-2-yl)-2-(hydroxymethyl)piperazin-1-yl)pyrimidine-5-carboxylic acid

To a solution of (R)-tert-butyl 2-(4-(4-amino-5-(ethoxycarbonyl)pyrimidin-2-yl)-3-(((tert-butyldiphenylsilyl)oxy)methyl)piperazin-1-yl)-7,8-dihydropyrido[4,3-d]pyrimidine-6(5H)-carboxylate (20.3 g, 26.96 mmol, 1.0 equiv) in THF (200 mL) was added TBAF (1.0 M, 50.75 mL, 1.9 equiv). The reaction mixture was stirred at room temperature for 5 h. The mixture was then poured into H₂O (200 mL) and the aqueous phase was extracted with EtOAc (2×150 mL). The combined organic phases were washed with brine (2×100 mL), dried, filtered and concentrated under reduced pressure. Purification by silica gel chromatography (0%→20% EtOAc/petroleum ether) afforded the desired product (12 g, 85.7% yield) as a white solid. LCMS (ESI) m/z: [M+H] calcd for C₂₄H₃₄N₈O₅: 515.28; found 515.4.

### Step 3: Synthesis of (R)-4-amino-2-(4-(6-(tert-butoxycarbonyl)-5,6,7,8-tetrahydropyrido[4,3-d]pyrimidin-2-yl)-2-(hydroxymethyl)piperazin-1-yl)pyrimidine-5-carboxylic acid

To a solution of (R)-4-amino-2-(4-(6-(tert-butoxycarbonyl)-5,6,7,8-tetrahydropyrido[4,3-d]pyrimidin-2-yl)-2-(hydroxymethyl)piperazin-1-yl)pyrimidine-5-carboxylic acid (12 g, 23.32 mmol, 1.0 equiv) in THF (100 mL), EtOH (30 mL), and H₂O (30 mL) was added LiOH·H₂O (5.87 g, 139.92 mmol, 6.0 equiv). The mixture was stirred at 50° C. for 22 h. The mixture was then concentrated under reduced pressure to remove THF and EtOH. The aqueous phase was neutralized with 1 N HCl and the resulting precipitate was filtered. The filter cake was washed with H₂O (50 mL) and dried under reduced pressure. The filtrate was extracted with DCM (8×60 mL) and the combined organic phases were washed with brine (2×50 mL), dried, filtered, and concentrated under reduced pressure. The resulting residue was combined with the initial filter cake and the solid was dissolved in DCM (150 mL) and concentrated under reduced pressure to afford the desired product (9.76 g, 85.2% yield) as a white solid. LCMS (ESI) m/z: [M+H] calcd for C₂₂H₃₀N₈O₅: 487.24; found 487.2.

### Building Block AF. 4-amino-2-[(2S)-4-{6-[(tert-butoxy)carbonyl]-5H,6H,7H,8H-pyrido[4,3-d]pyrimidin-2-yl}-2-(hydroxymethyl)piperazin-1-yl]pyrimidine-5-carboxylic acid

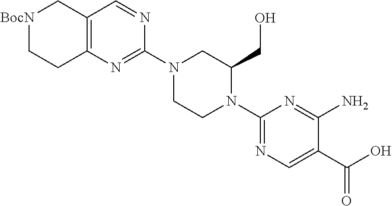 [SMILES: CC(C)(C)OC(=O)N1CCc2nc(N3CCN(c4ncc(C(=O)O)c(N)n4)[C@H](CO)C3)ncc2C1]

This building block is prepared from Building block O by a process similar to that for Building block J by utilizing ethyl 4-amino-2-chloropyrimidine-5-carboxylate.

### Building Block AG. 2-((2-(4-(5-((di-(tert-butoxycarbonyl)amino)methyl)pyrimidin-2-yl)piperazin-1-yl)-2-oxoethyl)(methyl)amino)acetic acid

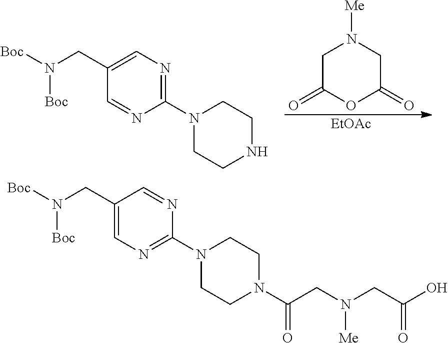

To a solution of tert-butyl N-tert-butoxycarbonyl-N-((2-piperazin-1-ylpyrimidin-5-yl)methyl)carbamate (4.88 g, 12.39 mmol, 1.0 equiv) in EtOAc (40 mL) was added 4-methylmorpholine-2,6-dione (1.6 g, 12.39 mmol, 1.0 equiv). The reaction was stirred at room temperature for 2 h then reaction mixture was concentrated under reduced pressure to give the crude product. The residue was triturated with EtOAc (15 mL) and filtered to give the product (5.65 g, 87.2% yield) as a white solid. LCMS (ESI) m/z: [M+H] calcd for C₂₄H₃₉N₆O₇: 523.28; found 523.3.

### Building Block AH. tert-butyl N-tert-butoxycarbonyl-N-((2-(4-(3-(2-piperazin-1-ylethoxy)propanoyl)piperazin-1-yl)pyrimidin-5-yl)methyl)carbamate

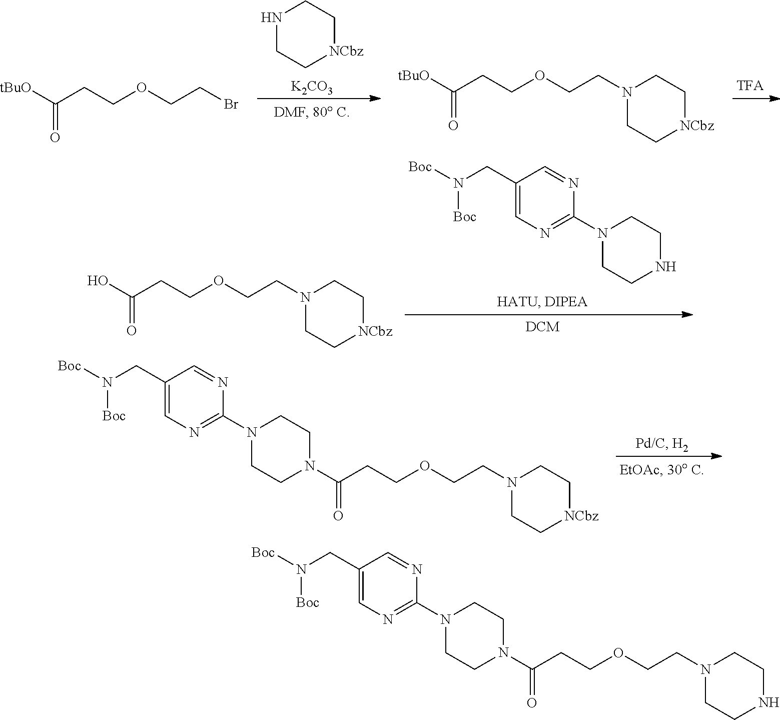

### Step 1: Synthesis of benzyl 4-(2-(3-(tert-butoxy)-3-oxopropoxy)ethyl)piperazine-1-carboxylate

To a solution of tert-butyl 3-(2-bromoethoxy)propanoate (35 g, 138.27 mmol, 1.0 equiv) and benzyl piperazine-1-carboxylate (31.14 mL, 138.27 mmol, 1.0 equiv, HCl) in MeCN (420 mL) was added K₂CO₃ (57.33 g, 414.80 mmol, 3.0 equiv). The reaction was stirred at 80° C. for 20 h. The reaction mixture was cooled to room temperature and the suspension was filtered. The filter cake was washed with EtOAc (3×50 mL) and the combined filtrates were concentrated under reduced pressure to give crude product. The residue was purified by silica gel chromatography (5/1 to 0/1 petroleum ether/EtOAc) to give the product (46 g, 84.8% yield) as a yellow oil.

### Step 2: Synthesis of 3-(2-(4-((benzyloxy)carbonyl)piperazin-1-yl)ethoxy)propanoic acid

A solution of benzyl 4-(2-(3-(tert-butoxy)-3-oxopropoxy)ethyl)piperazine-1-carboxylate (21 g, 53.50 mmol, 1.0 equiv) in TFA (160 mL) was stirred at room temperature for 2 h and then concentrated under reduced pressure. The residue was purified by silica gel chromatography (1/0 to 4/1 EtOAc/MeOH) to give the product (20.4 g, 84.7% yield) as a yellow oil. LCMS (ESI) m/z: [M+H] calcd for C₁₇H₂₄N₂O₅: 337.18; found 337.1.

### Step 3: Synthesis of benzyl 4-(2-(3-(4-(5-(((di-tert-butoxycarbonyl)amino)methyl)pyrimidin-2-yl)piperazin-1-yl)-3-oxopropoxy)ethyl)piperazine-1-carboxylate

To a solution of 3-(2-(4-((benzyloxy)carbonyl)piperazin-1-yl)ethoxy)propanoic acid (20.2 g, 44.85 mmol, 1.0 equiv, TFA) in DCM (500 mL) was added HATU (25.58 g, 67.27 mmol, 1.5 equiv) and DIPEA (17.39 g, 134.55 mmol, 23.44 mL, 3.0 equiv). The reaction was stirred at room temperature for 30 min, and then tert-butyl N-tert-butoxycarbonyl-N-((2-piperazin-1-ylpyrimidin-5-yl)methyl)carbamate (14.12 g, 35.88 mmol, 0.8 equiv) was added. The reaction mixture was stirred at for 2 h and then quenched with sat. NH₄Cl (500 mL). The aqueous phase was extracted with DCM (3×300 mL) and the combined organic phase was washed with brine (30 mL), dried with anhydrous Na₂SO₄, filtered and concentrated under reduced pressure to give crude product. The residue was purified by silica gel chromatography (0/1 petroleum ether/EtOAc to 10/1 DCM/MeOH) to give the product (29 g, 90.8% yield) as a yellow oil. LCMS (ESI) m/z: [M+H] calcd for C₃₆H₅₃N₇O₈: 712.41; found 712.4.

### Step 4: Synthesis of tert-butyl N-tert-butoxycarbonyl-N-((2-(4-(3-(2-piperazin-1-ylethoxy)propanoyl)piperazin-1-yl)pyrimidin-5-yl)methyl)carbamate

To a solution of 4-(2-(3-(4-(5-(((di-tert-butoxycarbonyl)amino)methyl)pyrimidin-2-yl)piperazin-1-yl)-3-oxopropoxy)ethyl)piperazine-1-carboxylate (5 g, 7.02 mmol, 1.0 equiv) in EtOAc (150 mL) was added Pd/C (2 g, 10 wt. %). The suspension was degassed and purged with H₂ and then stirred under H₂ (30 psi) at 30° C. for 3 h. The suspension was then cooled to room temperature and filtered through Celite. The filter cake was washed with MeOH (15×100 mL) and the combined filtrates were concentrated under reduced pressure to give the product (12 g, 89.9% yield) as a light yellow oil. LCMS (ESI) m/z: [M+H] calcd for C₂₈H₄₇N₇O₆: 578.37; found 578.5.

### Building Block AI. ethyl 2-(piperazin-1-yl)pyrimidine-5-carboxylate

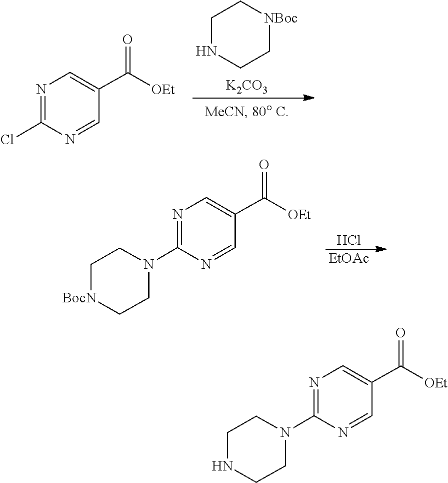

### Step 1: Synthesis of ethyl 2-(4-(tert-butoxycarbonyl)piperazin-1-yl)pyrimidine-5-carboxylate

To a solution of tert-butyl piperazine-1-carboxylate (11.94 g, 53.59 mmol, 1.0 equiv, HCl) and ethyl 2-chloropyrimidine-5-carboxylate (10 g, 53.59 mmol, 1.0 equiv) in MeCN (100 mL) was added K₂CO₃ (7.41 g, 53.59 mmol, 1.0 equiv). The mixture was stirred at 80° C. for 17 h and then poured into H₂O (200 mL). The mixture was filtered and the filter cake was washed with H₂O (80 mL) and dried under reduced pressure to give the product (15.76 g, 82% yield) as a white solid.

### Step 2: Synthesis of ethyl 2-(piperazin-1-yl)pyrimidine-5-carboxylate

To a solution of ethyl 2-(4-(tert-butoxycarbonyl)piperazin-1-yl)pyrimidine-5-carboxylate (15.7 g, 46.67 mmol, 1.0 equiv) in EtOAc (150 mL) was added HCl/EtOAc (150 mL) at 0° C. The resulting mixture was stirred at room temperature for 9 h. The reaction mixture was filtered and the filter cake was washed with EtOAc (100 mL). The solid was dried under reduced pressure to give the product (12.55 g, 96% yield, HCl) as a white solid. LCMS (ESI) m/z: [M+H] calcd for C₁₁H₁₆N₄O₂: 237.14; found 237.3.

### Building Block AJ. 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylic acid

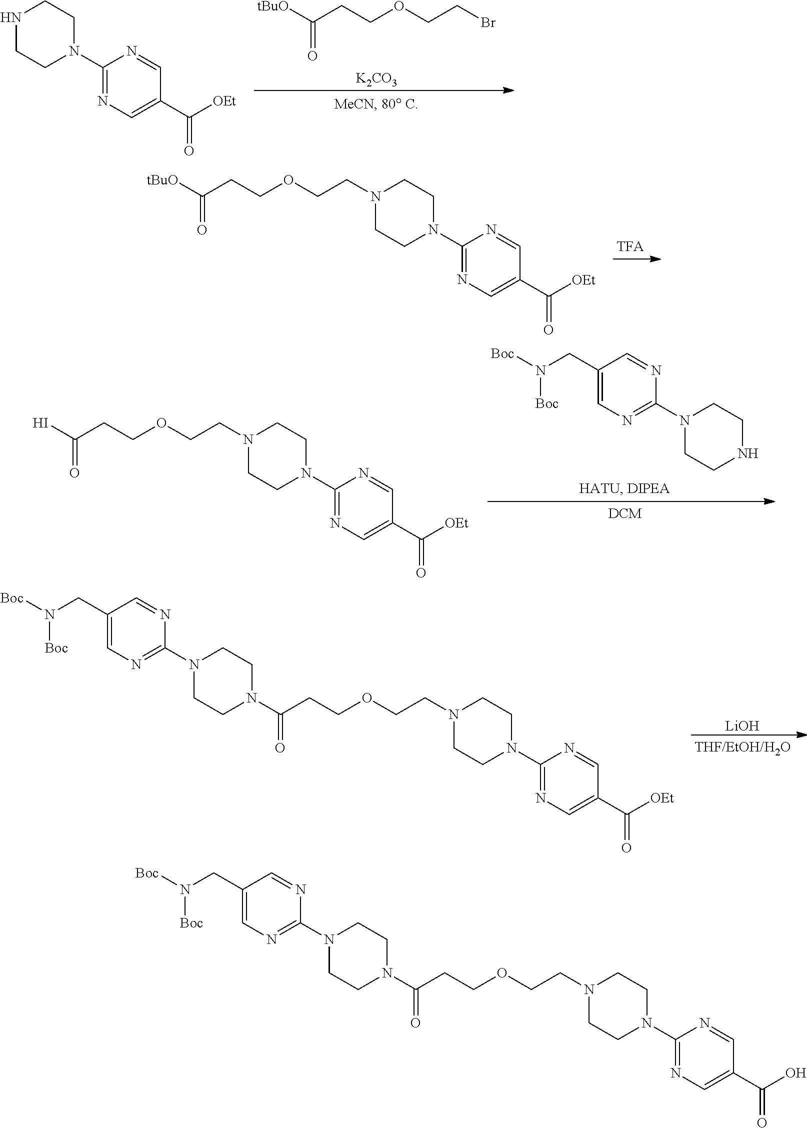

### Step 1: Synthesis of ethyl 2-(4-(2-(3-(tert-butoxy)-3-oxopropoxy)ethyl)piperazin-1-yl)pyrimidine-5-carboxylate

To a solution of ethyl 2-piperazin-1-ylpyrimidine-5-carboxylate (17.92 g, 75.85 mmol, 1.2 equiv) and tert-butyl 3-(2-bromoethoxy)propanoate (16 g, 63.21 mmol, 1.0 equiv) in MeCN (200 mL) was added K₂CO₃ (17.47 g, 126.42 mmol, 2.0 equiv). The reaction was stirred at 80° C. for 12 h and then the reaction mixture was filtered, and the filtrate was concentrated under reduced pressure. The crude product was suspended in petroleum ether (200 mL) and stirred for 20 min at 0° C. and then filtered. The solid was dried under reduced pressure to give the product (19.4 g, 75.1% yield) as a yellow solid.

### Step 2: Synthesis of 3-(2-(4-(5-(ethoxycarbonyl)pyrimidin-2-yl)piperazin-1-yl)ethoxy)propanoic acid

A solution of ethyl 2-(4-(2-(3-(tert-butoxy)-3-oxopropoxy)ethyl)piperazin-1-yl)pyrimidine-5-carboxylate (19.4 g, 47.49 mmol, 1.0 equiv) in TFA (200 mL) was stirred at room temperature for 30 min. The reaction mixture was then concentrated under reduced pressure and the residue was purified by silica gel chromatography (50/1 to 1/1 EtOAc/MeOH) to give the product (18 g, 81.3% yield) as a yellow oil.

### Step 3: Synthesis of ethyl 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylate

To a solution of 3-(2-(4-(5-(ethoxycarbonyl)pyrimidin-2-yl)piperazin-1-yl)ethoxy)propanoic acid (13 g, 27.87 mmol, 1.0 equiv) in DCM (200 mL) was added HATU (15.90 g, 41.81 mmol, 1.5 equiv) and DIPEA (19.42 mL, 111.49 mmol, 4.0 equiv). The reaction was then stirred at room temperature for 30 min and then tert-butyl N-tert-butoxycarbonyl-N-[(2-piperazin-1-ylpyrimidin-5-yl)methyl]carbamate (10.97 g, 27.87 mmol, 1.0 equiv) was added. The mixture was stirred at for 2 h and then poured into a sat. NH₄Cl solution (200 mL). The aqueous phase was extracted with DCM (2×200 mL) and the combined organic phase was washed with brine (2×20 mL), dried with anhydrous Na₂SO₄, filtered and concentrated under reduced pressure. The residue was purified by silica gel chromatography (100/1 to 9/1 EtOAc/MeOH) to give the product (17 g, 79.0% yield) as a yellow oil.

### Step 4: Synthesis of 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylic acid

To a solution of ethyl 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylate (11 g, 15.11 mmol, 1.0 equiv) in THF (40 mL), EtOH (10 mL), and H₂O (20 mL) was added LiOH·H₂O (1.27 g, 30.23 mmol, 2.0 equiv). The mixture was then stirred at 35° C. for 1.5 h. The reaction mixture was extracted with EtOAc (30 mL) and the aqueous phase was adjusted to pH=7 by addition of HCl (1 N). The mixture was then concentrated under reduced pressure. The crude product was purified by reversed-phase chromatography (20/1 to 3/1 H₂O/MeCN) to give the product (6.1 g, 67.3% yield) as a white solid. LCMS (ESI) m/z: [M+H] calcd for C₃₃H₄₉N₉O₈: 700.38; found 700.4.

### Building Block AK. 2-(4-(2-(3-(4-(5-(((tert-butoxycarbonyl)amino)methyl)pyrimidin-2-yl)piperazin-1-yl)-3-oxopropoxy)ethyl)piperazin-1-yl)pyrimidine-5-carboxylic acid

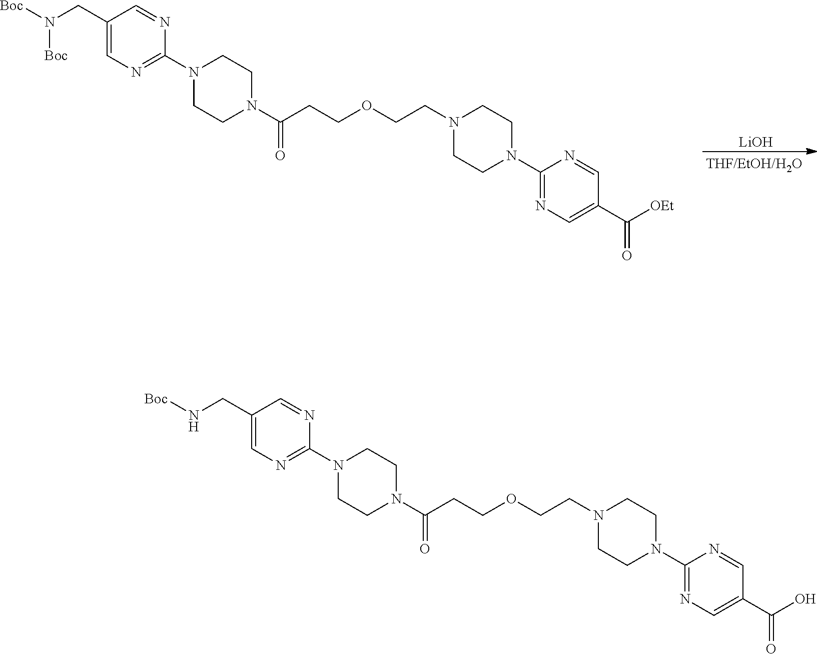 [SMILES: CCOC(=O)c1cnc(N2CCN(CCOCCC(=O)N3CCN(c4ncc(CN(C(=O)OC(C)(C)C)C(=O)OC(C)(C)C)cn4)CC3)CC2)nc1 ; "LiOH" ; CC(C)(C)OC(=O)NCc1cnc(N2CCN(C(=O)CCOCCN3CCN(c4ncc(C(=O)O)cn4)CC3)CC2)nc1]

A solution of ethyl 2-(4-(2-(3-(4-(5-(((di-tert-butoxycarbonyl)amino)methyl)pyrimidin-2-yl)piperazin-1-yl)-3-oxopropoxy)ethyl)piperazin-1-yl)pyrimidine-5-carboxylate (5.4 g, 7.42 mmol, 1.0 equiv) in THF (40 mL), EtOH (10 mL), and H₂O (10 mL) was added LiOH·H₂O (933.92 mg, 22.26 mmol, 3.0 equiv). The mixture was then stirred at 30° C. for 12 h. The reaction mixture was then extracted with EtOAc (2×50 mL) and the aqueous phase was adjusted to pH=7 by addition of HCl (1 N). The solution was then concentrated under reduced pressure. The crude product was purified by reversed-phase chromatography (20/1 to 3/1 H₂O/MeCN) to give the product (1.01 g, 22.5% yield) as a white solid. LCMS (ESI) m/z: [M+H] calcd for C₂₈H₄₁N₉O₆: 600.33; found 600.2.

### Building Block AL. 4-{4-[2-(3-{4-[5-({[(tert-butoxy)carbonyl]amino}methyl)pyrimidin-2-yl]piperazin-1-yl}-3-oxopropoxy)ethyl]piperazin-1-yl}-4-oxobutanoic acid

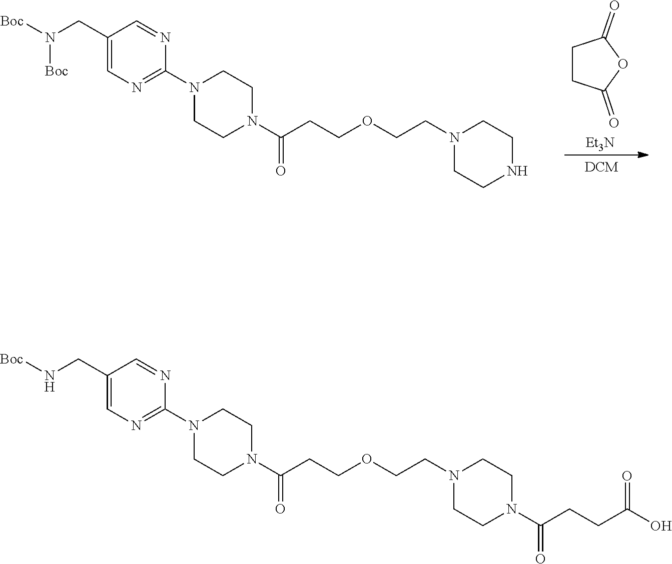 [SMILES: "Et3N" ; CC(C)(C)OC(=O)N(Cc1cnc(N2CCN(C(=O)CCOCCN3CCNCC3)CC2)nc1)C(=O)OC(C)(C)C ; O=C1CCC(=O)O1 ; CC(C)(C)OC(=O)NCc1cnc(N2CCN(C(=O)CCOCCN3CCN(C(=O)CCC(=O)O)CC3)CC2)nc1]

To a solution of tert-butyl N-tert-butoxycarbonyl-N-((2-(4-(3-(2-piperazin-1-ylethoxy)propanoyl)piperazin-1-yl)pyrimidin-5-yl)methyl)carbamate (1.0 equiv) in DCM is added succinic anhydride (1.2 equiv) and Et₃N (2.0 equiv). The reaction is stirred at room temperature until consumption of starting material, as determined by LCMS analysis. The reaction mixture is then concentrated under reduced pressure to give the crude product. The residue is purified by silica gel chromatography to afford the product.

### Building Block AM. 2-(4-(4-(4-(5-(((tert-butoxycarbonyl)amino)methyl)pyrimidin-2-yl)piperazin-1-yl)-4-oxobutyl)piperazin-1-yl)pyrimidine-5-carboxylic acid

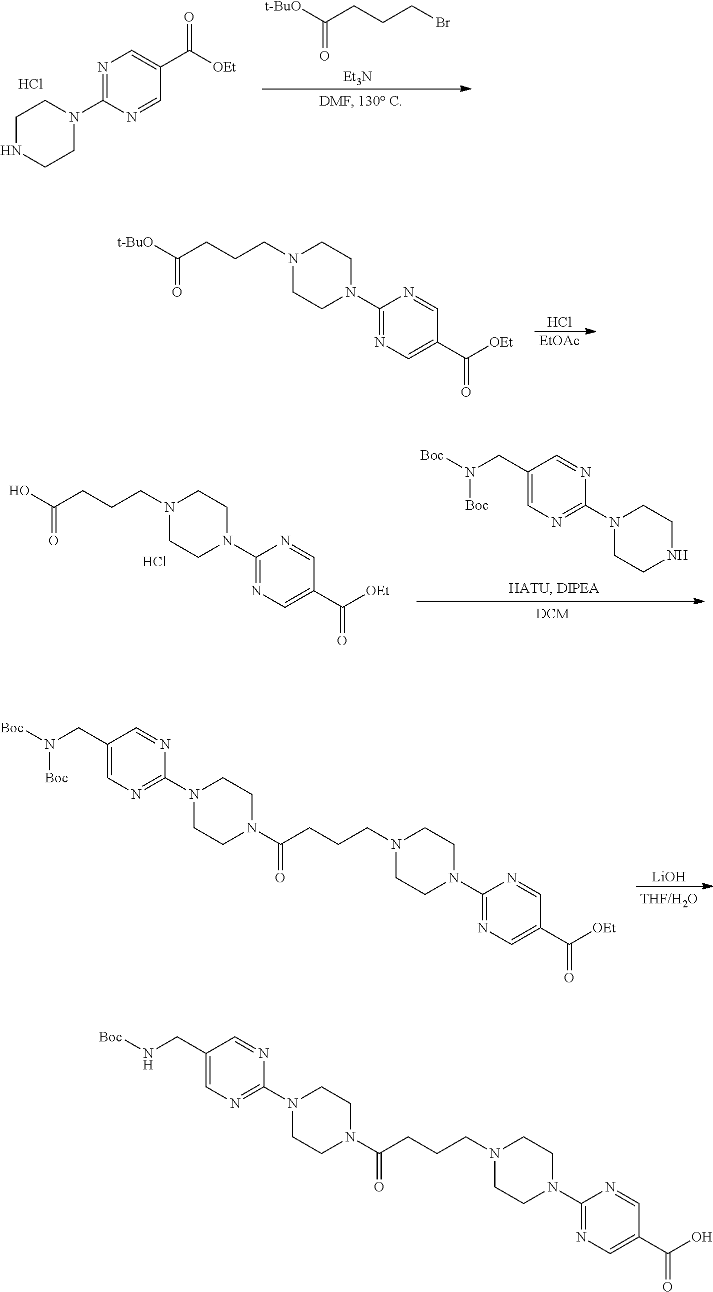

### Step 1: Synthesis of ethyl 2-(4-(4-(tert-butoxy)-4-oxobutyl)piperazin-1-yl)pyrimidine-5-carboxylate

To a solution of ethyl 2-(piperazin-1-yl)pyrimidine-5-carboxylate hydrochloride (10 g, 36.67 mmol, 1.0 equiv, HCl) and tert-butyl 4-bromobutanoate (8.18 g, 36.67 mmol, 1.0 equiv) in DMF (100 mL) was added Et₃N (15.31 mL, 110.00 mmol, 3.0 equiv). The mixture was stirred at 130° C. for 14 h. The mixture was then poured into H₂O (400 mL) and the solution was extracted with EtOAc (3×150 mL). The combined organic layer was washed with brine (200 mL), dried over Na₂SO₄ and concentrated under reduced pressure. The residue was purified by silica gel chromatography (5/1 to 1/1 petroleum ether/EtOAc) to give the product (9.5 g, 68.5% yield) as a yellow solid. LCMS (ESI) m/z: [M+H] calcd for C₁₉H₃₀N₄O₄: 379.24; found 379.2, 380.2.

### Step 2: Synthesis of 4-(4-(5-(ethoxycarbonyl)pyrimidin-2-yl)piperazin-1-yl)butanoic acid hydrochloride

To a solution of ethyl 2-(4-(4-(tert-butoxy)-4-oxobutyl)piperazin-1-yl)pyrimidine-5-carboxylate (9.5 g, 25.10 mmol, 1.0 equiv) in EtOAc (100 mL) was added HCl/EtOAc (500 mL). The mixture was stirred at room temperature for 10 h and then the solution was concentrated under reduced pressure to give the product (9.6 g, 96.8% yield) as a white solid. LCMS (ESI) m/z: [M+H] calcd for C₁₅H₂₂N₄O₄: 323.17; found 323.2.

### Step 3: Synthesis of ethyl 2-(4-(4-(4-(5-(((di-tert-butoxycarbonyl)amino)methyl)pyrimidin-2-yl)piperazin-1-yl)-4-oxobutyl)piperazin-1-yl)pyrimidine-5-carboxylic acid

To a solution of 4-(4-(5-(ethoxycarbonyl)pyrimidin-2-yl)piperazin-1-yl)butanoic acid hydrochloride (5 g, 15.51 mmol, 1.0 equiv) and tert-butyl N-tert-butoxycarbonyl-N-((2-piperazin-1-ylpyrimidin-5-yl)methyl)carbamate (6.10 g, 15.51 mmol, 1.0 equiv) in DMF (150 mL) was added DIPEA (8.11 mL, 46.53 mmol, 3.0 equiv) and HATU (7.08 g, 18.61 mmol, 1.2 equiv). The mixture was stirred at room temperature for 3 h and then the solution was poured into H₂O (600 mL). The aqueous layer was extracted with EtOAc (3×200 mL) and then the combined organic layer was washed with brine (100 mL), dried with Na₂SO₄ and concentrated under reduced pressure. The residue was purified by silica gel chromatography (50/1 to 15/1 DCM/MeOH) to give the product (6.3 g, 58.2% yield) as a yellow solid. LCMS (ESI) m/z: [M+H] calcd for C₃₄H₅₁N₉O₇: 698.40; found 698.6.

### Step 4: Synthesis of 2-(4-(4-(4-(5-(((tert-butoxycarbonyl)amino)methyl)pyrimidin-2-yl)piperazin-1-yl)-4-oxobutyl)piperazin-1-yl)pyrimidine-5-carboxylic acid

To a solution of ethyl 2-(4-(4-(4-(5-((bis(tert-butoxycarbonyl)amino)methyl)pyrimidin-2-yl)piperazin-1-yl)-4-oxo-butyl)piperazin-1-yl)pyrimidine-5-carboxylate (4.5 g, 6.45 mmol, 1.0 equiv) in EtOH (7 mL) and THF (28 mL) was added a solution of LiOH·H₂O (541.17 mg, 12.90 mmol, 2.0 equiv) in H₂O (7 mL). The mixture was stirred at 30° C. for 8 h, then additional LiOH·H₂O (541 mg, 12.90 mmol, 2.0 equiv) was added. After stirring for an additional 8 h at 30° C., the solution was concentrated under reduced pressure. H₂O (20 mL) was added and solution was adjusted to pH 3 with 1N HCl. The suspension was filtered and the solid dried under reduced pressure to give the product (3.2 g, 79.1% yield) as a white solid. LCMS (ESI) m/z: [M+H] calcd for C₂₇H₃₉N₉O₅: 570.32; found 570.3.

### Building Block AN. 2-(4-(2-(2-(4-(6-(tert-butoxycarbonyl)-5,6,7,8-tetrahydropyrido[4,3-d]pyrimidin-2-yl)piperazin-1-yl)ethoxy)ethyl)piperazin-1-yl)pyrimidine-5-carboxylic acid

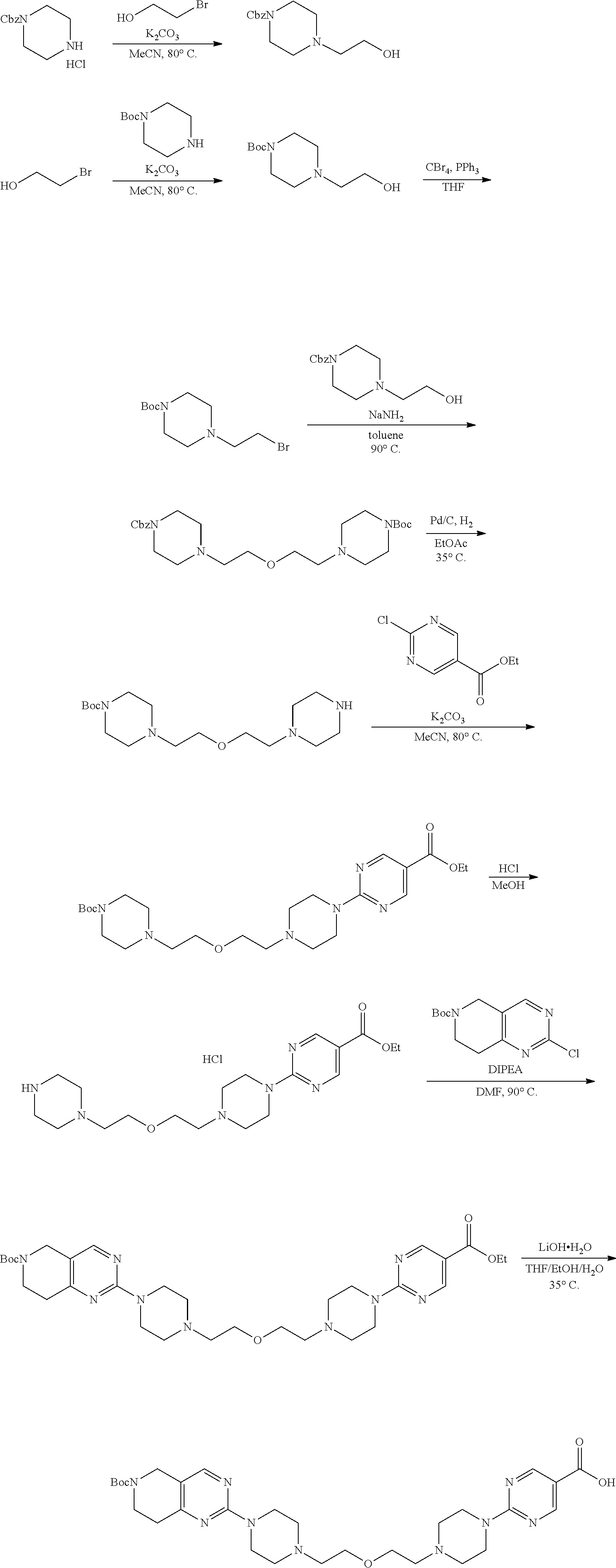

### Step 1: Synthesis of benzyl 4-(2-hydroxyethyl)piperazine-1-carboxylate

To a solution of benzyl piperazine-1-carboxylate hydrochloride (41.09 g, 160.04 mmol, 1.0 equiv, HCl) in MeCN (200 mL) was added K₂CO₃ (66.36 g, 480.13 mmol, 3.0 equiv) and 2-bromoethanol (20 g, 160.04 mmol, 1.0 equiv). The reaction mixture was stirred at 80° C. for 16 h, at which point it was cooled to room temperature and filtered. The filter cake was washed with EtOAc (100 mL) and the filtrate then washed with H₂O (100 mL). The aqueous phase was extracted with EtOAc (3×50 mL) and the combined organic phases were washed with brine (50 mL), dried, and concentrated under reduced pressure. Purification by silica gel chromatography (5→25% MeOH/EtOAc) afforded the desired product as a yellow solid (20 g, 47% yield). LCMS (ESI) m/z: [M+H] calcd for C₁₄H₂₀N₂O₃: 265.16; found 264.9.

### Step 2: Synthesis of tert-butyl 4-(2-hydroxyethyl)piperazine-1-carboxylate

To a solution of tert-butyl piperazine-1-carboxylate (198.72 g, 1.07 mol, 1.0 equiv) in MeCN (1500 mL) was added 2-bromoethanol (240 g, 1.92 mol, 1.8 equiv) and K₂CO₃ (221.19 g, 1.60 mol, 1.5 equiv). The reaction mixture was stirred at 80° C. for 16 h, at which point the mixture was cooled to room temperature, filtered, and the filtrate was concentrated under reduced pressure. Purification by silica gel chromatography (0→14% MeOH/EtOAc) afforded the desired product as a white solid (146 g, 59% yield).

### Step 3: Synthesis of tert-butyl 4-(2-bromoethyl)piperazine-1-carboxylate

To a solution of tert-butyl 4-(2-hydroxyethyl)piperazine-1-carboxylate (45 g, 195.39 mmol, 1.0 equiv) in THF (600 mL) was added triphenylphosphine (97.38 g, 371.25 mmol, 1.9 equiv) and CBr₄ (116.64 g, 351.71 mmol, 1.8 equiv). The mixture was stirred at room temperature for 3 h. Two separate batches were combined, and the reaction mixture was filtered, and the filtrate concentrated under reduced pressure. Purification by silica gel chromatography (1→25% EtOAc/petroleum ether) afforded the desired product as a light-yellow solid (31 g, 27% yield).

### Step 4: Synthesis of benzyl 4-(2-(2-(4-(tert-butoxycarbonyl)piperazin-1-yl)ethoxy)ethyl)piperazine-1-carboxylate

To a solution of benzyl 4-(2-hydroxyethyl)piperazine-1-carboxylate (18 g, 68.10 mmol, 1.0 equiv) in toluene (200 mL) was added NaNH₂ (26.57 g, 680.99 mmol, 10.0 equiv). tert-Butyl 4-(2-bromoethyl)piperazine-1-carboxylate (25 g, 85.27 mmol, 1.25 equiv) was added and the mixture was heated to 90° C. for 18 h. The mixture was cooled to room temperature and poured into H₂O (700 mL) at 0° C. The aqueous phase was extracted with EtOAc (3×240 mL) and the combined organic phases were washed successively with H₂O (350 mL) and sat. brine (2×200 mL), dried, filtered, and concentrated under reduced pressure. Purification by silica gel chromatography (0→12% MeOH/EtOAc) afforded the desired product as a light-yellow oil (20 g, 62% yield).

### Step 5: Synthesis of tert-butyl 4-(2-(2-(piperazin-1-yl)ethoxy)ethyl)piperazine-1-carboxylate

To a solution of benzyl 4-(2-(2-(4-(tert-butoxycarbonyl)piperazin-1-yl)ethoxy)ethyl)piperazine-1-carboxylate (20 g, 41.96 mmol, 1.0 equiv) in EtOAc (180 mL) was added Pd/C (8 g, 10 wt. %). The suspension was degassed under reduced pressure and purged with H₂ three times. The mixture was stirred under H₂ (30 psi) at 35° C. for 12 h. The reaction mixture was then filtered, and the filtrate was concentrated under reduced pressure. Purification by silica gel chromatography (0→100% MeOH/EtOAc) afforded the desired product as a colorless oil (10.8 g, 75% yield).

### Step 6: Synthesis of ethyl 2-(4-(2-(2-(4-(tert-butoxycarbonyl)piperazin-1-yl)ethoxy)-ethyl)piperazin-1-yl)pyrimidine-5-carboxylate

To a solution of tert-butyl 4-(2-(2-(piperazin-1-yl)ethoxy)ethyl)piperazine-1-carboxylate (10.8 g, 31.54 mmol, 1.0 equiv) in MeCN (100 mL) was added K₂CO₃ (13.08 g, 94.61 mmol, 3.0 equiv) and ethyl 2-chloropyrimidine-5-carboxylate (5.88 g, 31.54 mmol, 1.0 equiv). The mixture was stirred at 80° C. for 12 h, at which point the reaction was cooled to room temperature, filtered, and the filtrate concentrated under reduced pressure. Purification by silica gel chromatography (0→9% MeOH/DCM) afforded the desired product as a white solid (13.6 g, 85% yield).

### Step 7: Synthesis of 2-(4-(2-(2-(4-(6-(tert-butoxycarbonyl)-5,6,7,8-tetrahydropyrido-[4,3-d]pyrimidin-2-yl)piperazin-1-yl)ethoxy)ethyl)piperazin-1-yl)pyrimidine-5-carboxylic acid

To a solution of ethyl 2-(4-(2-(2-(4-(tert-butoxycarbonyl)piperazin-1-yl)ethoxy)ethyl)piperazin-1-yl)pyrimidine-5-carboxylate (13.6 g, 27.61 mmol, 1.0 equiv) in MeOH (50 mL) was added a solution of HCl in MeOH (4 M, 150 mL, 21.7 equiv). The reaction was stirred at room temperature for 4 h, at which point the mixture was concentrated under reduced pressure to afford the crude desired product as a white solid (13.8 g, 4HCl) that was taken directly onto the next step. LCMS (ESI) m/z: [M+H] calcd for C₁₉H₃₂N₆O₃: 393.26; found 393.3.

### Step 8: Synthesis of tert-butyl 2-(4-(2-(2-(4-(5-(ethoxycarbonyl)pyrimidin-2-yl)-piperazin-1-yl)ethoxy)ethyl)piperazin-1-yl)-7,8-dihydropyrido[4,3-d]pyrimidine-6(5H)-carboxylate

To a stirred solution of 2-(4-(2-(2-(4-(6-(tert-butoxycarbonyl)-5,6,7,8-tetrahydropyrido[4,3-d]pyrimidin-2-yl)piperazin-1-yl)ethoxy)ethyl)piperazin-1-yl)pyrimidine-5-carboxylic acid (10.2 g, 18.95 mmol, 1.0 equiv, 4HCl) and DIPEA (16.50 mL, 94.74 mmol, 5.0 equiv) in DMF (100 mL) was added tert-butyl 2-chloro-7,8-dihydropyrido[4,3-d]pyrimidine-6(5H)-carboxylate (5.11 g, 18.95 mmol, 1.0 equiv). The reaction mixture was stirred at 90° C. for 12 h. The reaction mixture was then cooled to room temperature and added to EtOAc (200 mL) and H₂O (400 mL). The aqueous phase was extracted with EtOAc (2×100 mL) and the combined organic phases were washed with aqueous NH₄Cl (4×100 mL), brine (2×100 mL), dried, filtered and concentrated under reduced pressure. Purification by silica gel chromatography (0→9% MeOH/DCM) afforded the desired product as a white solid (5.4 g, 45% yield). LCMS (ESI) m/z: [M+H] calcd for C₃₁H₄₇N₉O₅: 626.38; found 626.3.

### Step 9: Synthesis of 2-(4-(2-(2-(4-(6-(tert-butoxycarbonyl)-5,6,7,8-tetrahydropyrido[4,3-d]pyrimidin-2-yl)piperazin-1-yl)ethoxy)ethyl)piperazin-1-yl)pyrimidine-5-carboxylic acid

To a solution of tert-butyl 2-(4-(2-(2-(4-(5-(ethoxycarbonyl)pyrimidin-2-yl)piperazin-1-yl)ethoxy)ethyl)piperazin-1-yl)-7,8-dihydropyrido[4,3-d]pyrimidine-6(5H)-carboxylate (5.4 g, 8.63 mmol, 1.0 equiv) in THF (50 mL), EtOH (20 mL), and H₂O (20 mL) was added LiOH·H₂O (1.09 g, 25.89 mmol, 3.0 equiv). The reaction mixture was stirred at 35° C. for 12 h, at which point the mixture was concentrated under reduced pressure to remove THF and EtOH. The aqueous phase was neutralized to pH=7 with 0.5N HCl and concentrated under reduced pressure. Purification by reverse phase chromatography afforded the desired product as a white solid (4.72 g, 92% yield). LCMS (ESI) m/z: [M+H] calcd for C₂₉H₄₃N₉O₅: 598.35; found 598.3.

### Building Block AO. 1′-[(tert-butoxy)carbonyl]-[1,4′-bipiperidine]-4-carboxylic acid

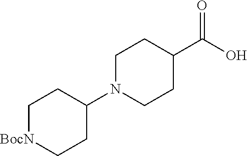 [SMILES: CC(C)(C)OC(=O)N1CCC(N2CCC(C(=O)O)CC2)CC1]

At the time of this application this building block was commercially available (CAS #201810-59-5).

### Building Block AP. 2-((2-(piperazin-1-yl)pyrimidin-5-yl)methyl)isoindoline-1,3-dione hydrochloride salt

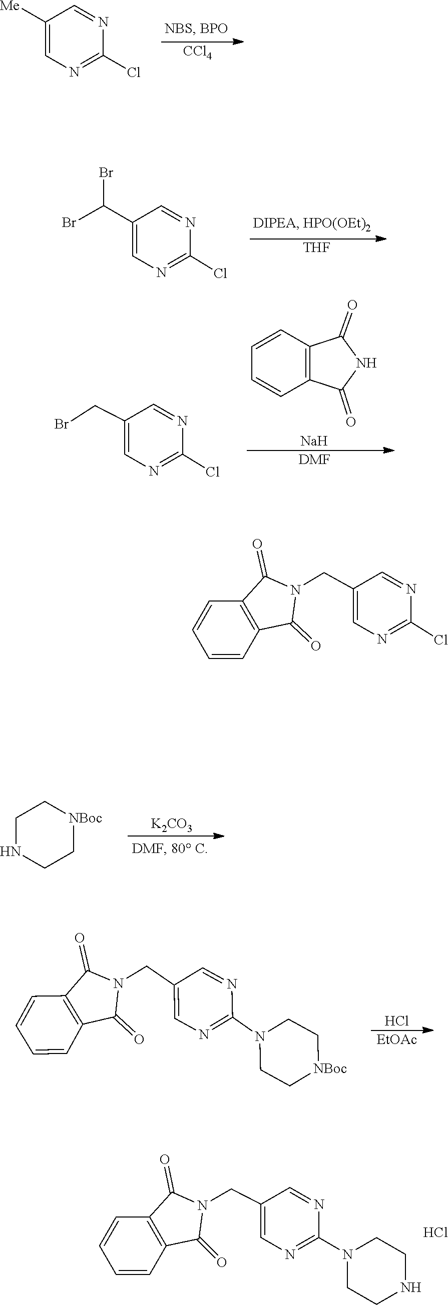

### Step 1: Synthesis of 2-chloro-5-(dibromomethyl)pyrimidine

To a solution of 2-chloro-5-methylpyrimidine (100 g, 777.85 mmol, 1.0 equiv) in CCl₄ (1200 mL) was added NBS (304.58 g, 1.71 mol, 2.2 equiv) and AIBN (51.09 g, 311.14 mmol, 0.4 equiv). The mixture was stirred at 80° C. for 16 h. The reaction solution was then cooled to room temperature, filtered, and the filtrate was poured into H₂O (1500 mL). The solution was diluted with DCM (3×250 mL) and the organic layer washed with brine (300 mL), dried with anhydrous Na₂SO₄, filtered and concentrated under reduced pressure to give the crude product as a brown oil, which was used directly in the next step.

### Step 2: Synthesis of 5-(bromomethyl)-2-chloropyrimidine

To a solution of 2-chloro-5-(dibromomethyl)pyrimidine (229 g, 799.72 mmol, 1.0 equiv) in THF (600 mL) was added DIPEA (111.44 mL, 639.77 mmol, 0.8 equiv) and 1-ethoxyphosphonoyloxyethane (82.57 mL, 639.77 mmol, 0.8 equiv). The mixture was stirred at room temperature for 19 h. The mixture was then poured into H₂O (1200 mL) and the aqueous phase was extracted with EtOAc (3×300 mL). The combined organic phase was washed with brine (300 mL), dried with anhydrous Na₂SO₄, filtered and concentrated under reduced pressure. The residue was purified by silica gel chromatography (1/0 to 0/1 petroleum ether/EtOAc) to give the product as a brown oil, which was used directly for the next step.

### Step 3: Synthesis of 2-((2-chloropyrimidin-5-yl)methyl)isoindoline-1,3-dione

To a mixture of isoindoline-1,3-dione (15 g, 101.95 mmol, 1.0 equiv) in DMF (126 mL) was added NaH (4.89 g, 122.34 mmol, 60 wt. %, 1.2 equiv) at 0° C. The mixture was stirred at 0° C. for 30 min, then a solution of 5-(bromomethyl)-2-chloro-pyrimidine (30.21 g, 101.95 mmol, 1.0 equiv) in DMF (24 mL) was added dropwise to the above mixture at room temperature. The mixture was stirred at room temperature for 2 h and was then cooled to 0° C. and quenched with sat. NH₄Cl (600 mL). The suspension was filtered and the solid dried under reduced pressure to give the crude product (27.4 g, 98.2% yield) as a grey solid, which was used directly in the next step. LCMS (ESI) m/z: [M+H] calcd for C₁₃H₈ClN₃O₂: 274.04; found 274.0.

### Step 4: Synthesis of tert-butyl 4-(5-((1,3-dioxoisoindolin-2-yl)methyl)pyrimidin-2-yl)piperazine-1-carboxylate

To a solution of 2-((2-chloropyrimidin-5-yl)methyl)isoindoline-1,3-dione (27 g, 98.66 mmol, 1.0 equiv) and tert-butyl piperazine-1-carboxylate (20.21 g, 108.52 mmol, 1.1 equiv) in DMF (270 mL) was added K₂CO₃ (34.09 g, 246.64 mmol, 2.5 equiv). The mixture was stirred at 80° C. for 3 h and then the reaction was cooled to room temperature and poured into H₂O (1200 mL). The suspension was filtered and the solid was dried under reduced pressure to give the crude product (35.58 g, 85.2% yield) as a white solid, which was used directly in the next step.

### Step 5: Synthesis of 2-((2-(piperazin-1-yl)pyrimidin-5-yl)methyl)isoindoline-1,3-dione

A solution of tert-butyl 4-(5-((1,3-dioxoisoindolin-2-yl)methyl)pyrimidin-2-yl)piperazine-1-carboxylate (15 g, 35.42 mmol, 1 equiv) in HCl/EtOAc (150 mL) was stirred at room temperature for 2 h. The mixture was filtered and then the filter cake was washed with EtOAc (20 mL) and dried under reduced pressure to give the product (42.53 g, 92.5% yield) as a white solid.

### Building Block AQ. 2-[(2-{4-[2-(3-{4-[5-({bis[(tert-butoxy)carbonyl]amino}methyl)pyrimidin-2-yl]piperazin-1-yl}-3-oxopropoxy)ethyl]piperazin-1-yl}-2-oxoethyl)(methyl)amino]acetic acid

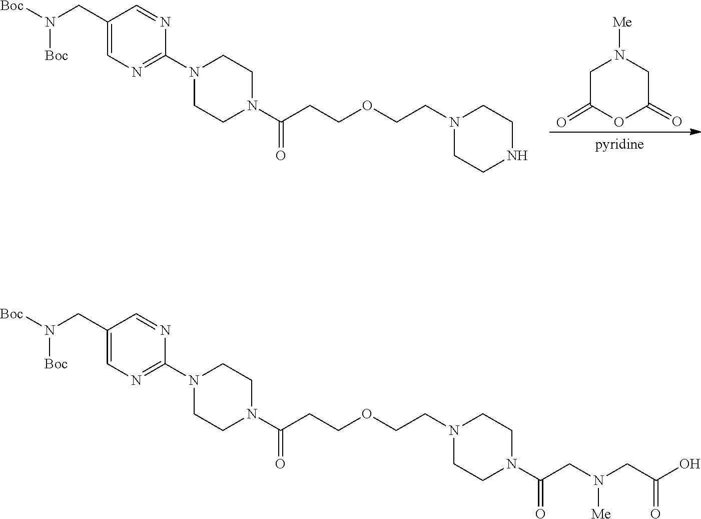 [SMILES: CC(C)(C)OC(=O)N(Cc1cnc(N2CCN(C(=O)CCOCCN3CCNCC3)CC2)nc1)C(=O)OC(C)(C)C ; CN1CC(=O)OC(=O)C1 ; CN(CC(=O)O)CC(=O)N1CCN(CCOCCC(=O)N2CCN(c3ncc(CN(C(=O)OC(C)(C)C)C(=O)OC(C)(C)C)cn3)CC2)CC1]

To a solution of tert-butyl N-[(tert-butoxy)carbonyl]-N-{[2-(4-{3-[2-(piperazin-1-yl)ethoxy]propanoyl}piperazin-1-yl)pyrimidin-5-yl]methyl}carbamate (300 mg, 519 μmol, 1.0 equiv) in pyridine (8 mL) at 0° C. was added 4-methylmorpholine-2,6-dione (80.3 mg, 622 μmol, 1.2 equiv). The reaction mixture was stirred at 0° C. for 1 h and then warmed to room temperature and stirred for an additional 12 h. The solvent was concentrated under reduced pressure and the solid was partitioned between DCM and H₂O. The organic layer was separated, dried over MgSO₄ and the solvent was concentrated under reduced pressure to give the product (23.0 mg, 6.28% yield). LCMS (ESI) m/z: [M+H] calcd for C₃₃H₅₄N₈O₉: 707.41; found 707.4.

### Building Block AR. 2-(4-(2-(3-(4-(6-(tert-butoxycarbonyl)-5,6,7,8-tetrahydropyrido[4,3-d]pyrimidin-2-yl)piperazin-1-yl)-3-oxopropoxy)ethyl)piperazin-1-yl)pyrimidine-5-carboxylic acid

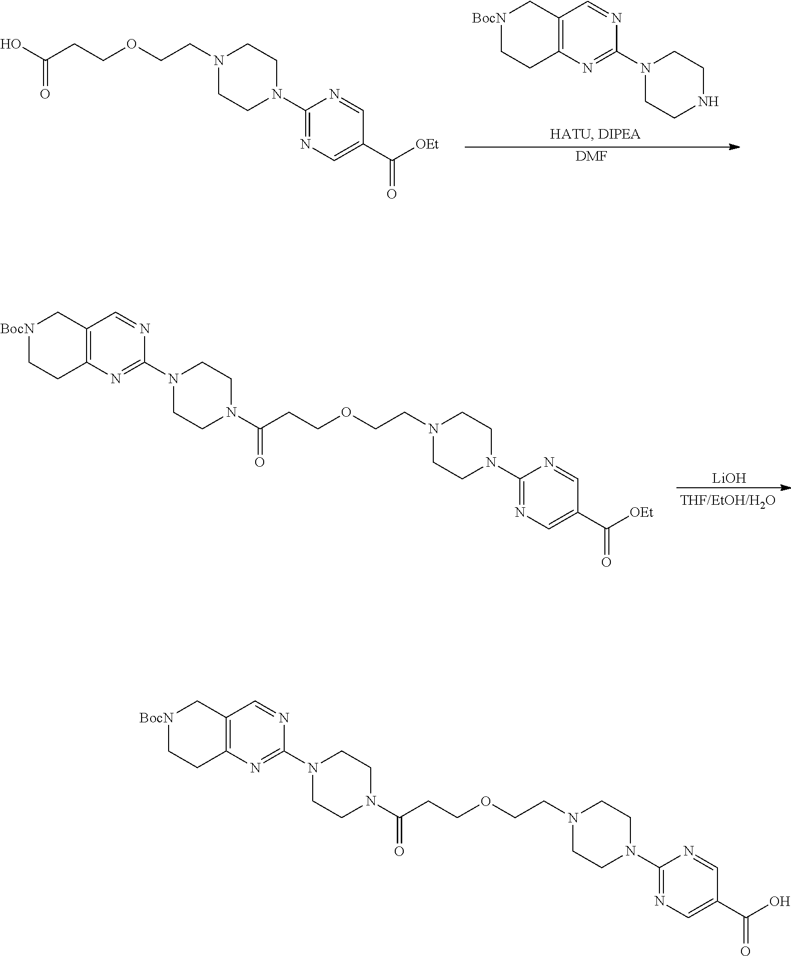

### Step 1: Synthesis of tert-butyl 2-(4-(3-(2-(4-(5-(ethoxycarbonyl)pyrimidin-2-yl)piperazin-1-yl)ethoxy)propanoyl)piperazin-1-yl)-7,8-dihydropyrido[4,3-d]pyrimidine-6(5H)-carboxylate

To a solution of 3-(2-(4-(5-(ethoxycarbonyl)pyrimidin-2-yl)piperazin-1-yl)ethoxy) propanoic acid (6 g, 12.86 mmol, 1.0 equiv, TFA) in DMF (55 mL) was added HATU (6.36 g, 16.72 mmol, 1.3 equiv) and DIPEA (11.20 mL, 64.32 mmol, 5.0 equiv). After 0.5 h, tert-butyl 2-(piperazin-1-yl)-7,8-dihydropyrido[4,3-d]pyrimidine-6(5H)-carboxylate (4.11 g, 12.86 mmol, 1.0 equiv) was added. The mixture was stirred for 3 h, at which point it was filtered and the solid cake was dried under reduced pressure to afford the desired product as a white solid (7.5 g, 89% yield). LCMS (ESI) m/z: [M+H] calcd for C₃₂H₄₇N₉O₆: 654.37; found 654.4.

### Step 2: Synthesis of 2-(4-(2-(3-(4-(6-(tert-butoxycarbonyl)-5,6,7,8-tetrahydropyrido[4,3-d]pyrimidin-2-yl)piperazin-1-yl)-3-oxopropoxy)ethyl)piperazin-1-yl)pyrimidine-5-carboxylic acid

To a solution of tert-butyl 2-(4-(3-(2-(4-(5-(ethoxycarbonyl)pyrimidin-2-yl)piperazin-1-yl)ethoxy)propanoyl)piperazin-1-yl)-7,8-dihydropyrido[4,3-d]pyrimidine-6(5H)-carboxylate (7.2 g, 11.01 mmol, 1.0 equiv) in THF (72 mL), EtOH (36 mL) and H₂O (36 mL) was added LiOH·H₂O (1.85 g, 44.05 mmol, 4.0 equiv). The reaction mixture was stirred at room temperature for 2.5 h, at which point the mixture was filtered and the filtrate was concentrated under reduced pressure to remove THF and EtOH. The aqueous phase was neutralized to pH=7 with 1N HCl, and then concentrated under reduced pressure. Purification by reverse phase chromatography (30% MeCN/H₂O) afforded the desired product as a white solid (3.85 g, 54% yield). LCMS (ESI) m/z: [M+H] calcd for C₃₀H₄₃N₉O₆: 626.34; found 626.3.

### Building Block AS. 2-(4-(2-(2-(3-(4-(5-((di-tert-butoxycarbonylamino)methyl)pyrimidin-2-yl)piperazin-1-yl)-3-oxo-propoxy)ethoxy)ethyl)piperazin-1-yl)pyrimidine-5-carboxylic acid

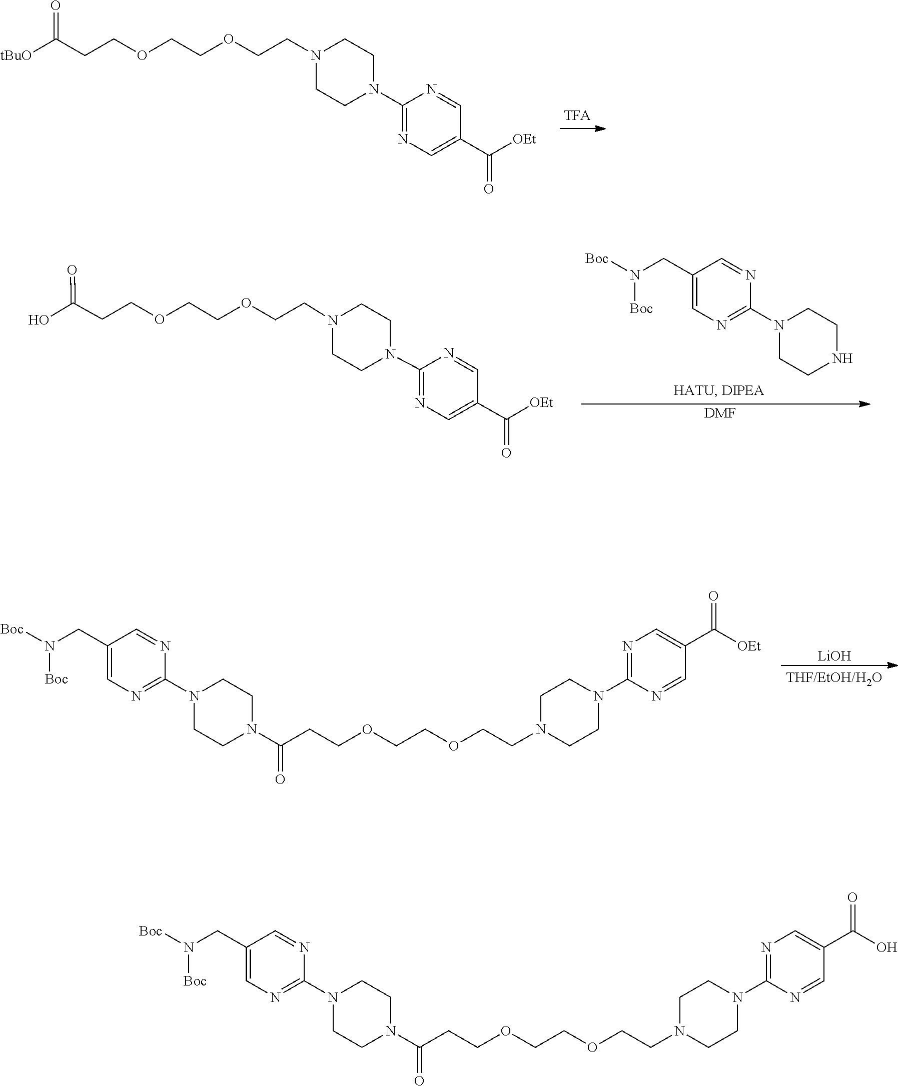

### Step 1: Synthesis of 3-(2-(2-(4-(5-(ethoxycarbonyl)pyrimidin-2-yl)piperazin-1yl)ethoxy)ethoxy)propanoic acid

A solution of ethyl 2-(4-(2-(2-(3-(tert-butoxy)-3-oxopropoxy)ethoxy)ethyl)piperazin-1-yl)pyrimidine-5-carboxylate (4 g, 8.84 mmol, 1.0 equiv) in TFA (12.29 mL, 166.00 mmol, 18.8 equiv) was stirred at room temperature for 3 h. The reaction mixture was concentrated under reduced pressure. Purification by silica gel chromatography (0→20% MeOH/EtOAc) afforded the desired product as a brown oil (4.35 g, 95% yield, TFA salt).

### Step 2: Synthesis of ethyl 2-(4-(2-(2-(3-(4-(5-((bis(tert-butoxycarbonyl)amino)methyl)pyrimidin-2-yl)piperazin-1-yl)-3-oxopropoxy)ethoxy)ethyl)piperazin-1-yl)pyrimidine-5-carboxylate

To a solution of 3-(2-(2-(4-(5-ethoxycarbonylpyrimidin-2-yl)piperazin-1-yl)ethoxy)ethoxy)propanoic acid (3.8 g, 7.44 mmol, 1.0 equiv, TFA) in DCM (30 mL) was added HATU (4.25 g, 11.17 mmol, 1.5 equiv) and DIPEA (6.48 mL, 37.22 mmol, 5.0 equiv). The reaction was stirred at room temperature for 30 min, and then tert-butyl N-tert-butoxycarbonyl-N-((2-piperazin-1ylpyrimidin-5-yl)methyl)carbamate (2.93 g, 7.44 mmol, 1.0 equiv) was added. The mixture was stirred at room temperature for 3.5 h, at which point the reaction mixture was concentrated under reduced pressure. Purification by silica gel chromatography (0→20% MeOH/EtOAc) afforded the desired product as a brown oil (4.14 g, 70% yield).

### Step 3: Synthesis of 2-(4-(2-(2-(3-(4-(5-((di-tert-butoxycarbonylamino)methyl)pyrimidin-2-yl)piperazin-1-yl)-3-oxo-propoxy)ethoxy)ethyl)piperazin-1-yl)pyrimidine-5-carboxylic acid

To a solution of ethyl 2-(4-(2-(2-(3-(4-(5-((bis(tert-butoxycarbonyl)amino)methyl)pyrimidin-2-yl)piperazin-1-yl)-3-oxo-propoxy)ethoxy)ethyl)piperazin-1-yl)pyrimidine-5-carboxylate (1.4 g, 1.81 mmol, 1.0 equiv) in THF (28 mL), EtOH (14 mL) and H₂O (14 mL) was added LiOH·H₂O (304.44 mg, 7.25 mmol, 4.0 equiv). The mixture was stirred at 40° C. for 30 min, at which point the reaction mixture was concentrated under reduced pressure. Purification by reverse phase chromatography (10→40% MeCN/H₂O) afforded the desired product as a yellow solid (500 mg, 43% yield).

### Building Block AT. 2-{4-[2-(2-{4-[5-({[(tert-butoxy)carbonyl]amino}methyl)pyrimidin-2-yl]piperazin-1-yl}ethoxy)ethyl]piperazin-1-yl}pyrimidine-5-carboxylic acid

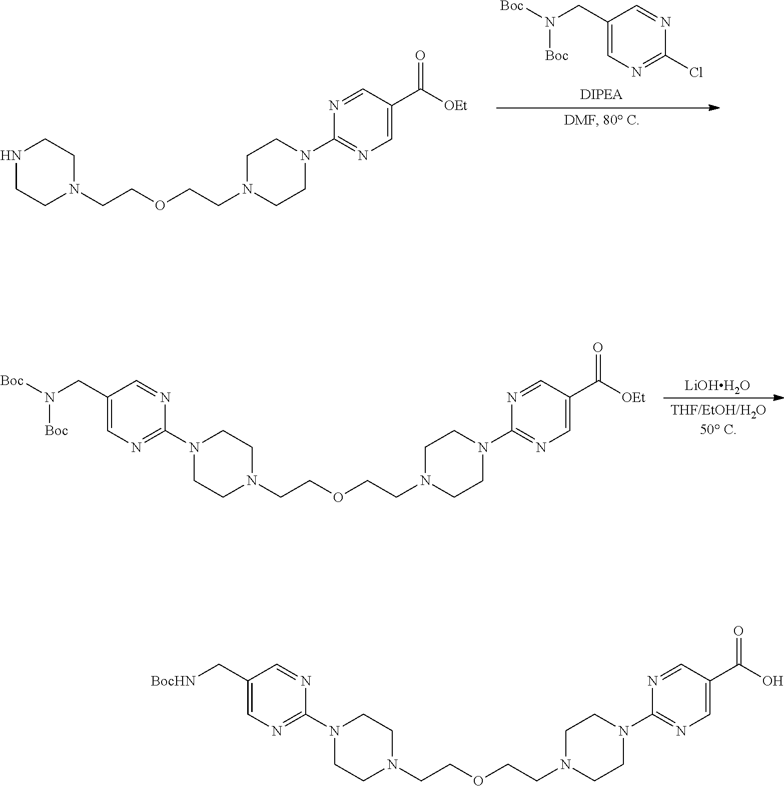 [SMILES: CCOC(=O)c1cnc(N2CCN(CCOCCN3CCNCC3)CC2)nc1 ; CC(C)(C)OC(=O)N(Cc1cnc(Cl)nc1)C(=O)OC(C)(C)C ; CCOC(=O)c1cnc(N2CCN(CCOCCN3CCN(c4ncc(CN(C(=O)OC(C)(C)C)C(=O)OC(C)(C)C)cn4)CC3)CC2)nc1 ; "LiOH·H2O" ; CC(C)(C)OC(=O)NCc1cnc(N2CCN(CCOCCN3CCN(c4ncc(C(=O)O)cn4)CC3)CC2)nc1]

### Step 1: Synthesis of ethyl 2-(4-(2-(2-(4-(5-(((di-tert-butoxycarbonyl)amino)methyl)pyrimidin-2-yl)piperazin-1-yl)ethoxy)ethyl)piperazin-1-yl)pyrimidine-5-carboxylate

To a solution of ethyl 2-(4-(2-(2-(piperazin-1-yl)ethoxy)ethyl)piperazin-1-yl)pyrimidine-5-carboxylate hydrochloride (7.3 g, 13.56 mmol, 1.0 equiv, 4HCl) in DMF (75 mL) was added DIPEA (14.17 mL, 81.36 mmol, 6.0 equiv) and tert-butyl-N-tert-butoxycarbonyl-N-[(2-chloropyrimidin-5-yl)methyl]carbamate (5.59 g, 16.27 mmol, 1.2 equiv). The mixture was stirred at 80° C. for 12 h. The mixture was then cooled to room temperature and poured into H₂O (300 mL). The aqueous phase was extracted with EtOAc (3×80 mL). The combined organic phases were washed with sat. NH₄Cl (4×80 mL) and brine (150 mL), dried, filtered and the filtrate was concentrated under reduced pressure. Purification by silica gel chromatography (0%→17% MeOH/EtOAc) afforded the desired product (7.7 g, 81.1% yield) as a light yellow oil. LCMS (ESI) m/z: [M+Na] calcd for C₃₄H₅₃N₉O₇: 722.40; found 722.4.

### Step 2: Synthesis of 2-(4-(2-(2-(4-(5-(((tert-butoxycarbonyl)amino)methyl)pyrimidin-2-yl)piperazin-1-yl)ethoxy)ethyl)piperazin-1-yl)pyrimidine-5-carboxylic acid

To a solution of ethyl 2-(4-(2-(2-(4-(5-(((di-tert-butoxycarbonyl)amino)methyl)pyrimidin-2-yl)piperazin-1-yl)ethoxy)ethyl)piperazin-1-yl)pyrimidine-5-carboxylate (7.7 g, 11.00 mmol, 1.0 equiv) in THF (80 mL), EtOH (20 mL), and H₂O (40 mL) was added LiOH·H₂O (2.31 g, 55.01 mmol, 5.0 equiv). The mixture was stirred at 50° C. for 26 h. The mixture was then concentrated under reduced pressure to remove THF and EtOH. The aqueous phase was neutralized with 0.5 N HCl, and concentrated under reduced pressure. Purification by reverse phase chromatography afforded the desired product (4.67 g, 74.3% yield) as a white solid. LCMS (ESI) m/z: [M−H] calcd for C₂₇H₄₁N₉O₅: 570.31; found 570.3.

### Building Block AU. (R)-tert-butyl 4-(5-(((tert-butoxycarbonyl-N-tert-butoxycarbonyl)amino)methyl)pyrimidin-2-yl)piperazine-2-carboxylate

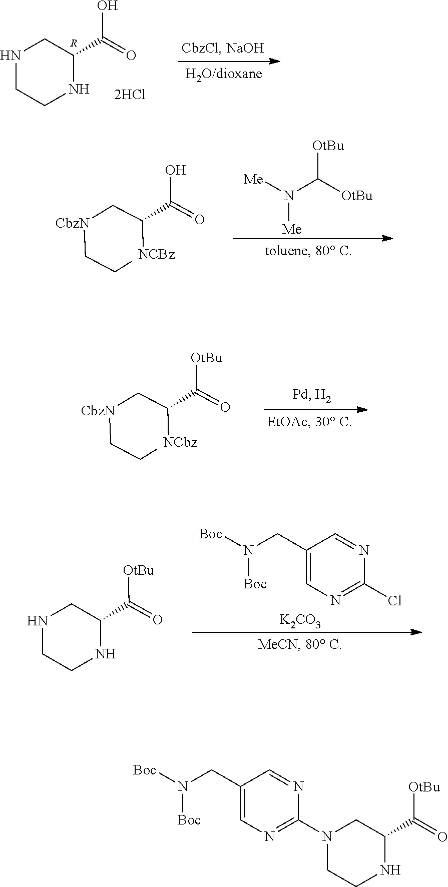

### Step 1: Synthesis of (R)-1,4-bis((benzyloxy)carbonyl)piperazine-2-carboxylic acid

To two separate batches containing a solution (2R)-piperazine-2-carboxylic acid (70 g, 344.71 mmol, 1 equiv, 2HCl) in H₂O (700 mL) and dioxane (1120 mL) was added 50% aq. NaOH until pH=11. Benzyl chloroformate (156.82 mL, 1.10 mol, 3.2 equiv) was added and the reaction was stirred at room temperature for 12 h. The two reaction mixtures were combined and H₂O (1200 mL) was added. The aqueous layer was extracted with MTBE (3×1000 mL), adjusted to pH=2 with con. HCl, and then extracted with EtOAc (2×1000 mL). The combined organic phases were dried, filtered, and concentrated under reduced pressure to afford the desired product (280 g, 86% yield). LCMS (ESI) m/z: [M+H] calcd for C₂₁H₂₂N₂O₆: 399.16; found 399.0.

### Step 2: Synthesis of (R)-1,4-dibenzyl 2-tert-butyl piperazine-1,2,4-tricarboxylate

To a solution of (R)-1,4-bis((benzyloxy)carbonyl)piperazine-2-carboxylic acid (70 g, 175.70 mmol, 1.0 equiv) in toluene (700 mL) at 80° C. was added 1,1-di-tert-butoxy-N,N-dimethyl-methanamine (80.04 mL, 333.83 mmol, 1.9 equiv). The reaction was stirred at 80° C. for 2 h, at which point it was cooled to room temperature and partitioned between EtOAc (300 mL) and H₂O (500 mL). The aqueous layer was extracted with EtOAc (2×500 mL) and the combined organic layers were dried, filtered, and concentrated under reduced pressure. Purification by silica gel chromatography (0→25 EtOAc/petroleum ether) afforded the desired product as a white solid (50 g, 57% yield). LCMS (ESI) m/z: [M+Na] calcd for C₂₅H₃₀N₂O₆: 477.20; found 476.9.

### Step 3: Synthesis of (R)-tert-butyl piperazine-2-carboxylate

To a solution of (R)-1,4-dibenzyl 2-tert-butyl piperazine-1,2,4-tricarboxylate (50 g, 110.01 mmol, 1 equiv) in EtOAc (20 mL) was added Pd/C (15 g, 10 wt. %). The suspension was degassed under reduced pressure and purged with H₂ three times. The suspension was stirred under H₂ (30 psi) at 30° C. for 4 h. The reaction mixture was then filtered, the residue was washed with MeOH (5×200 mL), and the filtrate concentrated under reduced pressure to afford the desired product as a yellow oil (17 g, 81% yield). LCMS (ESI) m/z: [M+H] calcd for C₉H₁₈N₂O₂: 187.15; found 187.1.

### Step 4: Synthesis of (R)-tert-butyl 4-(5-(((tert-butoxycarbonyl-N-tert-butoxycarbonyl)amino)methyl)pyrimidin-2-yl)piperazine-2-carboxylate

To a suspension of (R)-tert-butyl piperazine-2-carboxylate (8 g, 23.27 mmol, 1.0 equiv) and tert-butyl-N-tert-butoxycarbonyl ((2-chloropyrimidin-5-yl)methyl)carbamate (5.20 g, 27.92 mmol, 1.2 equiv) in MeCN (100 mL) was added K₂CO₃ (6.43 g, 46.54 mmol, 2.0 equiv). The reaction mixture was heated to 80° C. for 12 h, at which point it was cooled to room temperature, filtered, and the filtrate was concentrated under reduced pressure. Purification by silica gel chromatography (0→100% EtOAc/petroleum ether) afforded the desired product as a yellow solid (9.2 g, 73% yield). LCMS (ESI) m/z: [M+H] calcd for C₂₄H₃₉N₅O₆:494.30; found 494.1.

### Building Block AV. (S)-tert-butyl 4-(5-(((tert-butoxycarbonyl-N-tert-butoxycarbonyl)amino)methyl)pyrimidin-2-yl)piperazine-2-carboxylate

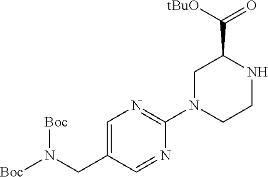 [SMILES: CC(C)(C)OC(=O)[C@@H]1CN(c2ncc(CN(C(=O)OC(C)(C)C)C(=O)OC(C)(C)C)cn2)CCN1]

This building block is prepared by a process similar to that for Building block AU by utilizing (2S)-piperazine-2-carboxylic acid.

### Building Block AW. (R)-2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylic acid

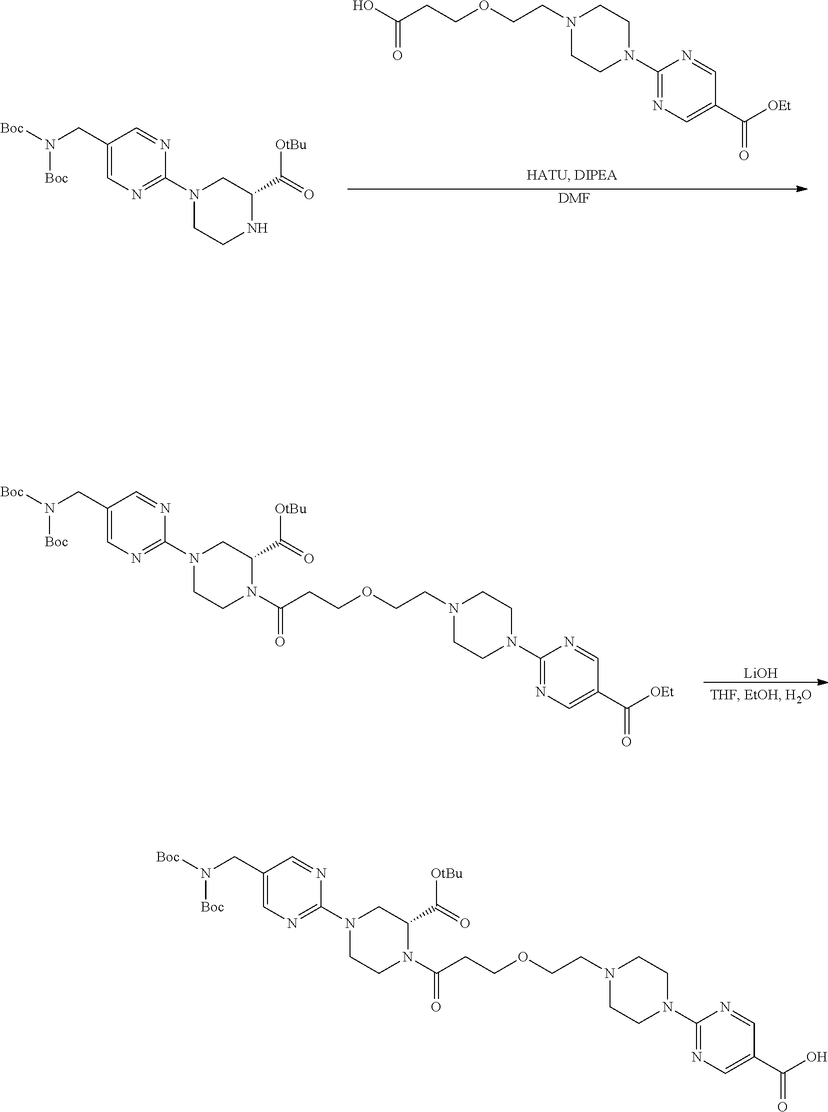

### Step 1: Synthesis of (R)-ethyl 2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylate

To a solution of (R)-tert-butyl 4-(5-(((tert-butoxycarbonyl-N-tert-butoxycarbonyl)amino)methyl)pyrimidin-2-yl)piperazine-2-carboxylate (5.3 g, 11.36 mmol, 1.0 equiv, TFA) in DCM (80 mL) was added HATU (6.48 g, 17.05 mmol, 1.5 equiv) and DIPEA (7.92 mL, 45.45 mmol, 4.0 equiv). The reaction was stirred at room temperature for 30 min and then tert-butyl (2R)-4-(5-((bis(tert-butoxycarbonyl)amino)methyl)pyrimidin-2-yl)piperazine-2-carboxylate (5.61 g, 11.36 mmol, 1.0 equiv) was added. The mixture was stirred for 1 h, at which point sat. NH₄Cl (80 mL) was added. The organic phase was washed with sat. NH₄Cl (5×80 mL), dried, filtered, and concentrated under reduced pressure. Purification by silica gel chromatography (0→9% MeOH/EtOAc) afforded the desired product as a yellow solid (8.4 g, 85% yield).

### Step 2: Synthesis of (R)-2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylic acid

To two separate batches containing a solution a solution of (R)-ethyl 2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylate (3.4 g, 4.11 mmol, 1.0 equiv) in THF (16 mL), EtOH (8 mL) and H₂O (8 mL) was added LiOH·H₂O (344.61 mg, 8.21 mmol, 2.0 equiv). The mixture was stirred at room temperature for 2 h. The two reaction mixtures were then combined and were adjusted to pH=7 with 1N HCl. The solution was concentrated under reduced pressure to remove THF and EtOH. The solution was then filtered, and the resulting solid was purified by reverse phase chromatography to afford the desired product as a white solid (4 g, 59% yield). LCMS (ESI) m/z: [M+H] calcd for C₃₈H₅₇N₉O₁₀: 800.43; found 800.3.

### Building Block AX. (S)-2-(4-(2-(3-(2-(tert-butoxycarbonyl)-4-(5-(((tert-butoxycarbonyl-N-tert-butoxycarbonyl)amino)methyl)pyrimidin-2-yl)piperazin-1-yl)-3-oxopropoxy)ethyl)piperazin-1-yl)pyrimidine-5-carboxylic acid

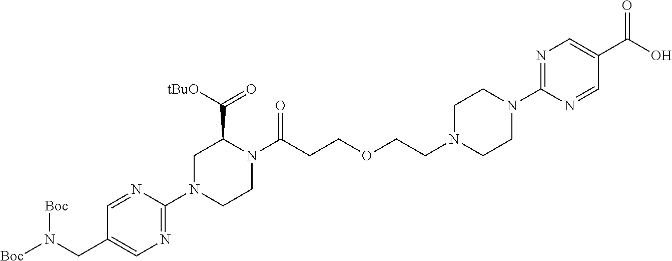 [SMILES: CC(C)(C)OC(=O)[C@@H]1CN(c2ncc(CN(C(=O)OC(C)(C)C)C(=O)OC(C)(C)C)cn2)CCN1C(=O)CCOCCN1CCN(c2ncc(C(=O)O)cn2)CC1]

This building block is prepared from Building block AV by a process similar to that for Building block AW.

### Building Block AY. 1′-(2-(3-(4-(5-(((tert-butoxycarbonyl)amino)methyl)pyrimidin-2-yl) piperazin-1-yl)-3-oxopropoxy)ethyl)-[1,4′-bipiperidine]-4-carboxylic acid

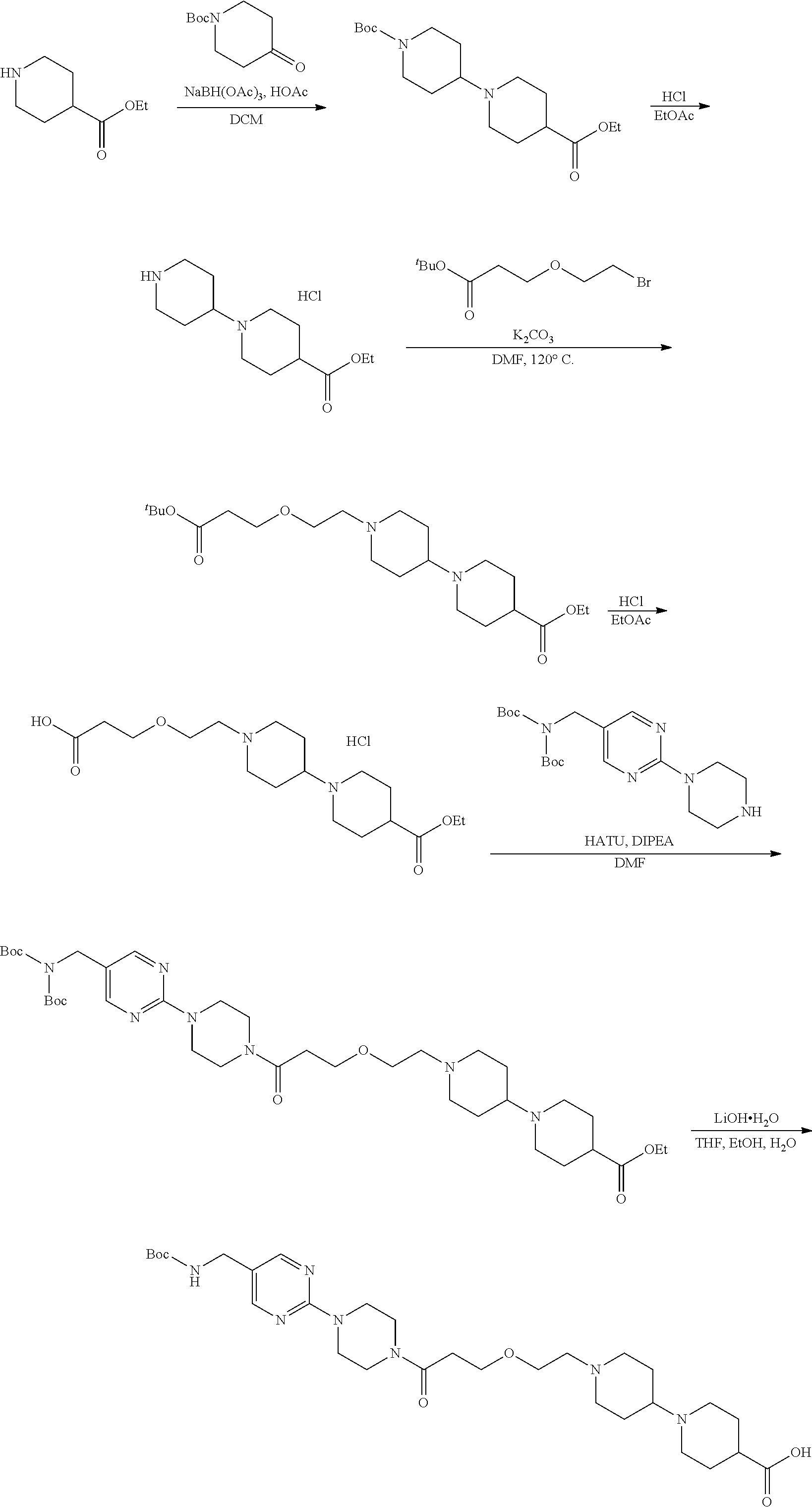

### Step 1: Synthesis of 1′-tert-butyl 4-ethyl [1,4′-bipiperidine]-1′,4-dicarboxylate

To a solution of ethyl piperidine-4-carboxylate (30 g, 150.57 mmol, 1.0 equiv) and tert-butyl 4-oxopiperidine-1-carboxylate (23.67 g, 150.57 mmol, 1.0 equiv) in DCM (300 mL) was added HOAc (6.00 mL, 104.95 mmol, 0.7 equiv). The mixture was stirred at room temperature for 30 min, then NaBH(OAc)₃ (63.82 g, 301.13 mmol, 2.0 equiv) was added. The mixture was stirred for 16 h, at which point H₂O (50 mL) was added. The aqueous phase was extracted with DCM (3×15 mL) and the combined organic phases were washed with brine (10 mL), dried, filtered, and concentrated under reduced pressure. Purification by silica gel chromatography (8→100 MeOH/EtOAc) afforded the desired product as a yellow oil (30 g, 59% yield).

### Step 2: Synthesis of ethyl [1,4′-bipiperidine]-4-carboxylate

To a solution of HCl in EtOAc (200 mL) was added 1′-tert-butyl 4-ethyl [1,4′-bipiperidine]-1′,4-dicarboxylate (20 g, 58.74 mmol, 1.0 equiv). The mixture was stirred at room temperature for 3 h. The mixture was then concentrated under reduced pressure to afford the desired crude product as a white solid (15 g, HCl salt).

### Step 3: Synthesis of ethyl 1′-(2-(3-(tert-butoxy)-3-oxopropoxy)ethyl)-[1,4′-bipiperidine]-4-carboxylate

To a solution of tert-butyl 3-(2-bromoethoxy)propanoate (6.46 g, 25.54 mmol, 1.0 equiv) in DMF (240 mL) was added K₂CO₃ (10.59 g, 76.61 mmol, 3.0 equiv) and ethyl [1,4′-bipiperidine]-4-carboxylate (8 g, 25.54 mmol, 1.0 equiv, 2HCl). The mixture was stirred at 120° C. for 12 h, at which point the reaction was cooled to room temperature, filtered, the filter cake washed with H₂O (20 mL), and the filtrate was concentrated under reduced pressure. Purification by silica gel chromatography (0→11% MeOH/EtOAc) afforded the desired product as a yellow oil (6.6 g, 63% yield).

### Step 4: Synthesis of 3-(2-(4-(ethoxycarbonyl)-[1,4′-bipiperidin]-1′-yl)ethoxy)propanoic acid

To the solution of HCl in EtOAc (70 mL) was added ethyl 1′-(2-(3-(tert-butoxy)-3-oxopropoxy) ethyl)-[1,4′-bipiperidine]-4-carboxylate (6.6 g, 16.00 mmol, 1.0 equiv). The mixture was stirred at room temperature for 3 h, at which point the reaction was concentrated under reduced pressure to afford the desired product as a white solid (6.5 g, 95% yield, 2HCl).

### Step 5: Synthesis of ethyl 1′-(2-(3-(4-(5-(((N,N-di-tert-butoxycarbonyl)amino)methyl)pyrimidin-2-yl)piperazin-1-yl)-3-oxopropoxy)ethyl)-[1,4′-bipiperidine]-4-carboxylate

To a solution of tert-butyl-tert-butoxycarbonyl((2-(piperazin-1-yl)pyrimidin-5-yl)methyl)carbamate (2.49 g, 6.33 mmol, 1.5 equiv) in DMF (40 mL) was added DIPEA (9.74 mL, 55.89 mmol, 6.0 equiv) and HATU (5.31 g, 13.97 mmol, 1.5 equiv). The mixture was stirred at room temperature for 30 min, and then 3-(2-(4-(ethoxycarbonyl)-[1,4′-bipiperidin]-1′-yl)ethoxy) propanoic acid (4 g, 9.32 mmol, 1.0 equiv, 2HCl) was added. The mixture was stirred at for 1.5 h, at which point H₂O (5 mL) and EtOAc (20 mL) were added. The aqueous phase was extracted with EtOAc (3×10 mL) and the combined organic phases were washed with brine (5 mL), dried, filtered and concentrated under reduced pressure. Purification by reverse phase chromatography afforded the desired product as a brown oil (1.6 g, 23% yield). LCMS (ESI) m/z: [M+H] calcd for C₃₇H₆₁N₇O₈: 732.47; found 732.6.

### Step 6: Synthesis of 1′-(2-(3-(4-(5-(((tert-butoxycarbonyl)amino)methyl)pyrimidin-2-yl)piperazin-1-yl)-3-oxopropoxy)ethyl)-[1,4′-bipiperidine]-4-carboxylic acid

To a solution of ethyl 1′-(2-(3-(4-(5-(((N,N-di-tert-butoxycarbonyl)amino)methyl)pyrimidin-2-yl)piperazin-1-yl)-3-oxopropoxy)ethyl)-[1,4′-bipiperidine]-4-carboxylate (1.4 g, 1.91 mmol, 1.0 equiv) in THF (7.5 mL), EtOH (3.8 mL), and H₂O (3.8 mL) was added LiOH·H₂O (321.07 mg, 7.65 mmol, 4.0 equiv). The mixture was stirred at room temperature for 2 h, at which point the mixture was concentrated under reduced pressure. Purification by reverse phase chromatography (5→38% MeCN/H₂O) afforded the desired product as a yellow solid (325 mg, 22% yield). LCMS (ESI) m/z: [M+H] calcd for C₃₀H₄₉N₇O₆: 604.38; found 604.3.

### Building Block AZ. 1-(4-{2-[2-(2-{[(benzyloxy)carbonyl]amino}ethoxy)ethoxy]ethyl}piperazin-1-yl)-3,6,9,12-tetraoxapentadecan-15-oic acid

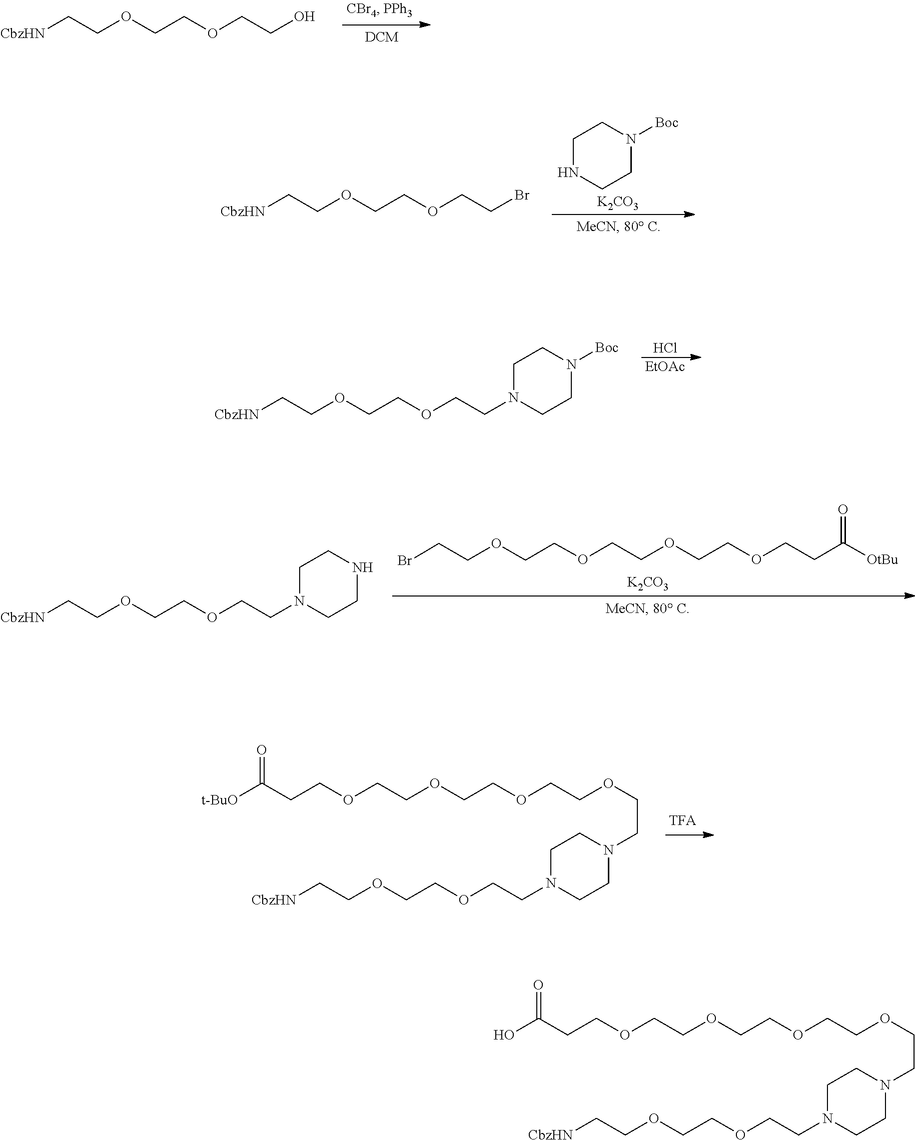

### Step 1: Synthesis of benzyl (2-(2-(2-bromoethoxy)ethoxy)ethyl)carbamate

To a solution of benzyl (2-(2-(2-hydroxyethoxy)ethoxy)ethyl)carbamate (10 g, 35.30 mmol, 1.0 equiv) in DCM (300 mL) at 0° C. was added PPh₃ (13.79 g, 52.59 mmol, 1.49 equiv) and CBr₄ (17.44 g, 52.59 mmol, 1.49 equiv). Then the mixture was warmed to room temperature and stirred for 12 h. The reaction mixture was then filtered, and the filtrate was concentrated under reduced pressure. Purification by silica gel chromatography (1%→25% EtOAc/petroleum ether) afforded the desired product (10.8 g, 88.4% yield) as yellow oil.

### Step 2: Synthesis of tert-butyl 4-(3-oxo-1-phenyl-2,7,10-trioxa-4-azadodecan-12-yl)piperazine-1-carboxylate

To a solution of benzyl (2-(2-(2-bromoethoxy)ethoxy)ethyl)carbamate (10.8 g, 31.19 mmol, 1.0 equiv) and tert-butyl piperazine-1-carboxylate (5.81 g, 31.19 mmol, 1.0 equiv) in MeCN (100 mL) was added K₂CO₃ (4.31 g, 31.19 mmol, 1.0 equiv). The mixture was stirred at 80° C. for 1 h. The reaction mixture was then filtered, and the filtrate was concentrated under reduced pressure. Purification by silica gel chromatography (0%→50% MeOH/EtOAc) afforded the desired product (13.1 g, 93.0% yield) as yellow oil.

### Step 3: Synthesis of benzyl (2-(2-(2-(piperazin-1-yl)ethoxy)ethoxy)ethyl)carbamate

A solution of tert-butyl 4-(3-oxo-1-phenyl-2,7,10-trioxa-4-azadodecan-12-yl)piperazine-1-carboxylate (5.64 g, 12.49 mmol, 1.0 equiv) in HCl/EtOAc (50 mL, 4 M) was stirred at room temperature for 1 h. The reaction mixture was then concentrated under reduced pressure to afford the desired product (5.23 g, crude, HCl salt) as yellow oil.

### Step 4: Synthesis of tert-butyl 1-(4-(3-oxo-1-phenyl-2,7,10-trioxa-4-azadodecan-12-yl)piperazin-1-yl)-3,6,9,12-tetraoxapentadecan-15-oate

A solution of benzyl (2-(2-(2-(piperazin-1-yl)ethoxy)ethoxy)ethyl)carbamate (13.3 g, 31.34 mmol, 1.0 equiv, 2HCl) and tert-butyl 1-bromo-3,6,9,12-tetraoxapentadecan-15-oate in MeCN (150 mL) was added K₂CO₃ (21.66 g, 156.71 mmol, 5.0 equiv). The mixture was stirred at 80° C. for 12 h. The reaction mixture was then filtered, and the filtrate was concentrated under reduced pressure. Purification by silica gel chromatography (1%→17% MeOH/DCM) afforded the desired product (5.4 g, 26.3% yield) as a yellow oil.

### Step 5: Synthesis of 1-(4-(3-oxo-1-phenyl-2,7,10-trioxa-4-azadodecan-12-yl)piperazin-1-yl)-3,6,9,12-tetraoxapentadecan-15-oic acid

A solution of tert-butyl 1-(4-(3-oxo-1-phenyl-2,7,10-trioxa-4-azadodecan-12-yl)piperazin-1-yl)-3,6,9,12-tetraoxapentadecan-15-oate (2.4 g, 3.66 mmol, 1.0 equiv) in TFA (20 mL) was stirred at room temperature for 30 min. The reaction mixture was then concentrated under reduced pressure to afford the desired product (3.03 g, TFA salt) as yellow oil.

### Building Block BA. (R)-2-(2-(tert-butoxycarbonyl)-4-(6-(tert-butoxycarbonyl)-5,6,7,8-tetrahydropyrido[4,3-d]pyrimidin-2-yl)piperazin-1-yl)pyrimidine-5-carboxylic acid

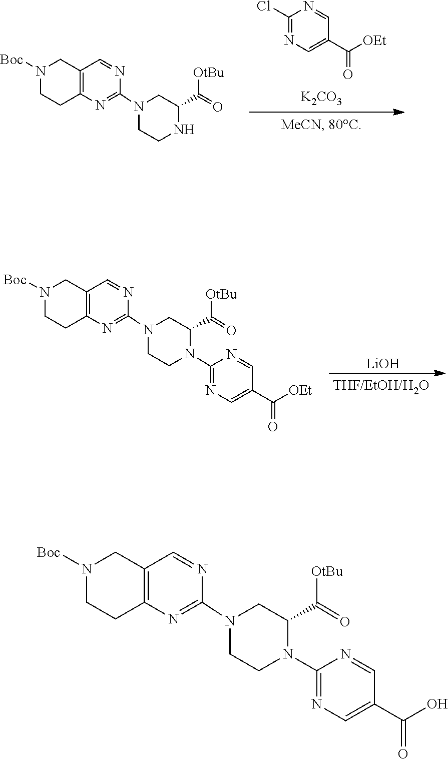 [SMILES: CC(C)(C)OC(=O)[C@H]1CN(c2ncc3c(n2)CCN(C(=O)OC(C)(C)C)C3)CCN1 ; "K2CO3" ; CCOC(=O)c1cnc(Cl)nc1 ; CCOC(=O)c1cnc(N2CCN(c3ncc4c(n3)CCN(C(=O)OC(C)(C)C)C4)C[C@@H]2C(=O)OC(C)(C)C)nc1 ; "LiOH" ; CC(C)(C)OC(=O)[C@H]1CN(c2ncc3c(n2)CCN(C(=O)OC(C)(C)C)C3)CCN1c1ncc(C(=O)O)cn1]

### Step 1: Synthesis of (R)-tert-butyl 2-(3-(tert-butoxycarbonyl)-4-(5-(ethoxycarbonyl)pyrimidin-2-yl)piperazin-1-yl)-7,8-dihydropyrido[4,3-d]pyrimidine-6(5H)-carboxylate

To two separate batches run in parallel each containing a solution of (R)-tert-butyl 2-(3-(tert-butoxycarbonyl)piperazin-1-yl)-7,8-dihydropyrido[4,3-d]pyrimidine-6(5H)-carboxylate (6 g, 14.30 mmol, 1.0 equiv) and K₂CO₃ (3.95 g, 28.60 mmol, 2.0 equiv) in MeCN (80 mL) was added ethyl 2-chloropyrimidine-5-carboxylate (3.20 g, 17.16 mmol, 1.2 equiv). The reaction mixtures were stirred at 80° C. for 12 h. The two reactions mixtures were combined and filtered, the residue was washed with EtOAc (3×50 mL), and the filtrate was concentrated under reduced pressure. Purification by silica gel chromatography (0%→17% MeOH/EtOAc) afforded the desired product (15 g, 91.5% yield) as a yellow solid. LCMS (ESI) m/z: [M+H] calcd for C₂₈H₃₉N₇O₆: 570.31; found 570.1.

### Step 2: Synthesis of (R)-2-(2-(tert-butoxycarbonyl)-4-(6-(tert-butoxycarbonyl)-5,6,7,8-tetrahydropyrido[4,3-d]pyrimidin-2-yl)piperazin-1-yl)pyrimidine-5-carboxylic acid

To a solution of (R)-tert-butyl 2-(3-(tert-butoxycarbonyl)-4-(5-(ethoxycarbonyl)pyrimidin-2-yl)piperazin-1-yl)-7,8-dihydropyrido[4,3-d]pyrimidine-6(5H)-carboxylate (15 g, 26.33 mmol, 1.0 equiv) in THF (80 mL), EtOH (40 mL) and H₂O (40 mL) was added LiOH·H₂O (2.21 g, 52.66 mmol, 2.0 equiv). The mixture was stirred at room temperature for 6 h. The reaction mixture was then adjusted to pH=6 with 1 N HCl. The resulting suspension was filtered, and the solid cake was dried under reduced pressure to afford the desired product (10.87 g, 75.9% yield) as a white solid. LCMS (ESI) m/z: [M+H] calcd for C₂₆H₃₅N₇O₆: 542.27; found 542.1.

### Building Block BB. (S)-2-(2-(tert-butoxycarbonyl)-4-(6-(tert-butoxycarbonyl)-5,6,7,8-tetrahydropyrido[4,3-d]pyrimidin-2-yl)piperazin-1-yl)pyrimidine-5-carboxylic acid

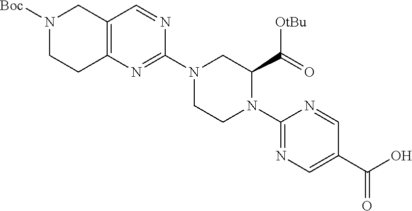 [SMILES: CC(C)(C)OC(=O)[C@@H]1CN(c2ncc3c(n2)CCN(C(=O)OC(C)(C)C)C3)CCN1c1ncc(C(=O)O)cn1]

This building block is prepared from Building block AA by a process similar to that for Building block BA.

### Building Block BC. 2-[(2R)-2-[(tert-butoxy)carbonyl]-4-[5-({[(tert-butoxy)carbonyl]amino}methyl)pyrimidin-2-yl]piperazin-1-yl]pyrimidine-5-carboxylic acid

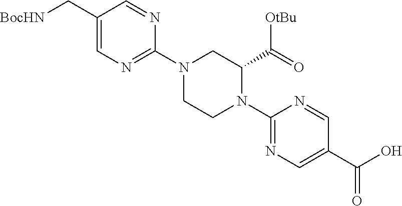 [SMILES: CC(C)(C)OC(=O)NCc1cnc(N2CCN(c3ncc(C(=O)O)cn3)[C@@H](C(=O)OC(C)(C)C)C2)nc1]

This building block is prepared from Building block AU by a process similar to that for Building block BA.

### Building Block BD. 2-[(2S)-2-[(tert-butoxy)carbonyl]-4-[5-({[(tert-butoxy)carbonyl]amino}methyl)pyrimidin-2-yl]piperazin-1-yl]pyrimidine-5-carboxylic acid

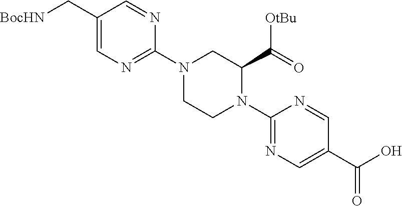 [SMILES: CC(C)(C)OC(=O)NCc1cnc(N2CCN(c3ncc(C(=O)O)cn3)[C@H](C(=O)OC(C)(C)C)C2)nc1]

This building block is prepared from Building block AV by a process similar to that for Building block BA.

### Building Block BE. 15-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-1-((1S,4S)-5-(2-(2-(2-aminoethoxy)ethoxy)ethyl)-2,5-diazabicyclo[2.2.1]heptan-2-yl)-3,6,9,12-tetraoxapentadecan-15-one

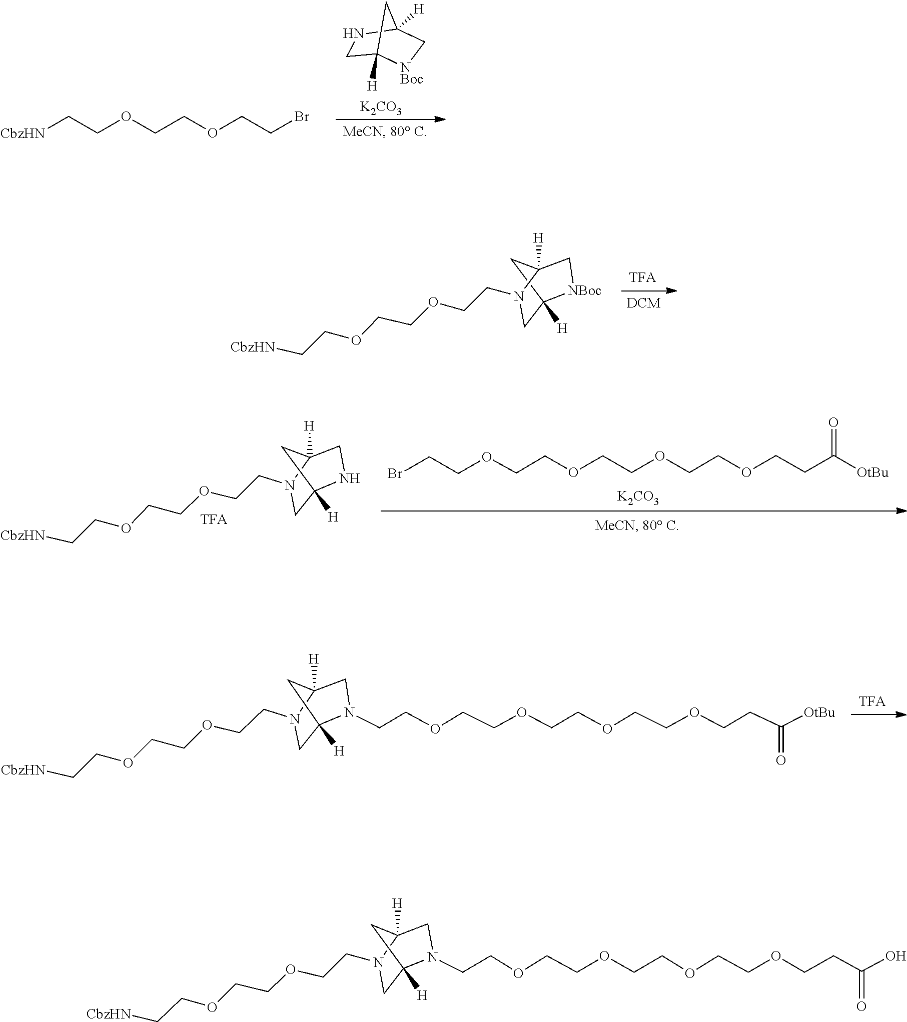

### Step 1: Synthesis of (1S,4S)-tert-butyl 5-(3-oxo-1-phenyl-2,7,10-trioxa-4-azadodecan-12-yl)-2,5-diazabicyclo[2.2.1]heptane-2-carboxylate

To a solution of (1S,4S)-tert-butyl 2,5-diazabicyclo[2.2.1]heptane-2-carboxylate (2.85 g, 14.37 mmol, 1.0 equiv) in MeCN (50 mL) was added K₂CO₃ (3.97 g, 28.75 mmol, 2.0 equiv) and benzyl (2-(2-(2-bromoethoxy)ethoxy)ethyl)carbamate (4.98 g, 14.37 mmol, 1.0 equiv). The mixture was stirred at 80° C. for 24 h. The reaction mixture was then filtered, and the filtrate was concentrated under reduced pressure. Purification by silica gel chromatography (0→10% MeOH/EtOAc) afforded the desired product (6.2 g, 93.0% yield) as colorless oil. LCMS (ESI) m/z: [M+H] calcd for C₂₄H₃₇N₃O₆: 464.27; found 464.2.

### Step 2: Synthesis of benzyl (2-(2-(2-((1S,4S)-2,5-diazabicyclo[2.2.1]heptan-2-yl)ethoxy)ethoxy)ethyl)carbamate

To a solution of (1S,4S)-tert-butyl 5-(3-oxo-1-phenyl-2,7,10-trioxa-4-azadodecan-12-yl)-2,5-diazabicyclo[2.2.1]heptane-2-carboxylate (6.2 g, 13.37 mmol, 1.0 equiv) in DCM (60 mL) was added TFA (20.7 mL, 279.12 mmol, 20.9 equiv). The reaction was stirred for 2 h, at which point the mixture was concentrated under reduced pressure at 45° C. to afford the desired crude product (10.5 g, 4TFA) as light brown oil, which was used the next step directly. LCMS (ESI) m/z: [M+H] calcd for C₁₉H₂₉N₃O₄: 364.22; found 364.2.

### Step 3: Synthesis of tert-butyl 1-((1S,4S)-5-(3-oxo-1-phenyl-2,7,10-trioxa-4-azadodecan-12-yl)-2,5-diazabicyclo[2.2.1]heptan-2-yl)-3,6,9,12-tetraoxapentadecan-15-oate

To a solution of benzyl (2-(2-(2-((1S,4S)-2,5-diazabicyclo[2.2.1]heptan-2-yl)ethoxy) ethoxy)ethyl)carbamate (5 g, 6.10 mmol, 1.0 equiv, 4TFA) in MeCN (80 mL) was added K₂CO₃ (5.06 g, 36.61 mmol, 6.0 equiv) and tert-butyl 1-bromo-3,6,9,12-tetraoxapentadecan-15-oate (2.35 g, 6.10 mmol, 1.0 equiv). The reaction mixture was stirred at 80° C. for 12 h. The reaction mixture was then filtered, and the filtrate was concentrated under reduced pressure. Purification by silica gel chromatography (0→15% MeOH/EtOAc) afforded the desired product (5.2 g, 92.8% yield) as light yellow oil. LCMS (ESI) m/z: [M+H] calcd for C₃₄H₅₇N₃O₁₀: 668.4; found 668.4.

### Step 4: Synthesis of 1-((1S,4S)-5-(3-oxo-1-phenyl-2,7,10-trioxa-4-azadodecan-12-yl)-2,5-diazabicyclo[2.2.1]heptan-2-yl)-3,6,9,12-tetraoxapentadecan-15-oic acid

A solution of tert-butyl 1-((1S,4S)-5-(3-oxo-1-phenyl-2,7,10-trioxa-4-azadodecan-12-yl)-2,5-diazabicyclo[2.2.1]heptan-2-yl)-3,6,9,12-tetraoxapentadecan-15-oate (5.2 g, 5.66 mmol, 1.0 equiv) in TFA (47.3 mL, 638.27 mmol, 112.75 equiv) was stirred at room temperature for 30 min. The mixture was then concentrated under reduced pressure at 45° C. Purification by reverse phase chromatography (2→35% MeCN/H₂O(0.05% NH₄OH)) afforded the desired product (1.88 g, 54.3% yield) as light brown oil. LCMS (ESI) m/z: [M+H] calcd for C₃₀H₄₉N₃O₁₀: 612.34; found 612.3.

### Building Block BF. 21-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-1-(piperazin-1-yl)-3,6,9,12,15,18-hexaoxahenicosan-21-one

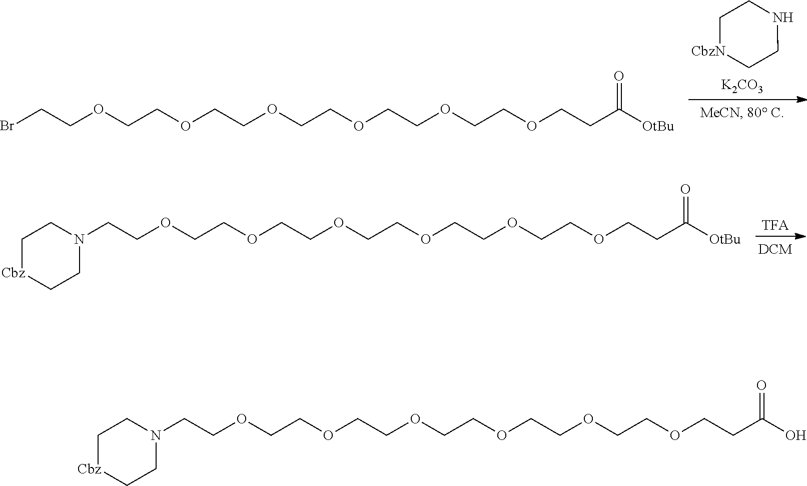

### Step 1: Synthesis of benzyl 4-(23,23-dimethyl-21-oxo-3,6,9,12,15,18,22-heptaoxatetracosyl) piperazine-1-carboxylate

To a solution of tert-butyl 1-bromo-3,6,9,12,15,18-hexaoxahenicosan-21-oate (5 g, 10.56 mmol, 1.0 equiv) and benzyl piperazine-1-carboxylate (2.62 mL, 11.62 mmol, 1.1 equiv, HCl) in MeCN (50 mL) was added K₂CO₃ (4.38 g, 31.69 mmol, 3.0 equiv). The reaction mixture was stirred at 80° C. for 10 h. The mixture was then filtered, the solid cake washed with EtOAc (3×3 mL), and the filtrate concentrated under reduced pressure. Purification by silica gel chromatography (0→10% MeOH/EtOAc) afforded the desired product (4 g, 61.8% yield) as a red liquid.

### Step 2: Synthesis of 1-(4-((benzyloxy)carbonyl)piperazin-1-yl)-3,6,9,12,15,18-hexaoxahenicosan-1-oic acid

To a solution of benzyl 4-(23,23-dimethyl-21-oxo-3,6,9,12,15,18,22-heptaoxatetracosyl)piperazine-1-carboxylate (1.8 g, 2.94 mmol, 1.0 equiv) in DCM(10 mL) was added TFA (10 mL). The solution was stirred for 0.5 h. The solution was then concentrated under reduced pressure. To the residue was added DCM (30 mL) and then the solution was concentrated under reduced pressure to afford the desired product (1.6 g, 2.87 mmol, TFA) as a red liquid.

### Preparation of Rapamycin Monomers

### Monomer 1. 40(R)—O-(4-nitrophenyl)carbonate rapamycin

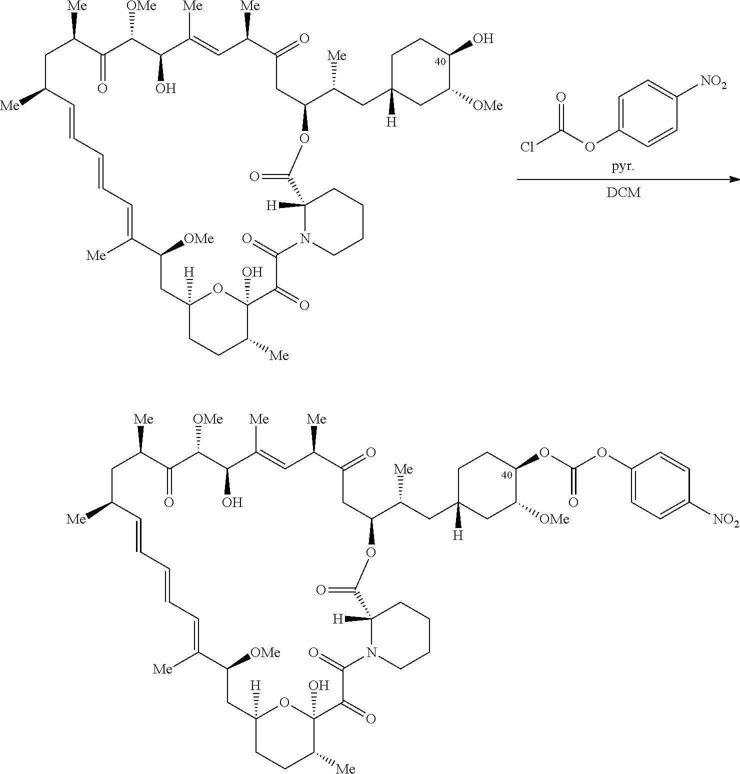

To a solution of rapamycin (30.10 g, 32.92 mmol, 1.0 equiv) in DCM (148.9 mL) was added pyridine (29.6 mL, 367 mmol, 11.1 equiv). The solution was cooled to −78° C. and then p-nitrophenyl chloroformate (12.48 g, 61.92 mmol, 1.9 equiv) was added. The reaction was stirred at −78° C. for 2 h. To the reaction mixture was then added DCM and the solution was then poured into H₂O. The aqueous layer was extracted with DCM and the combined organic layers were dried over MgSO₄, and concentrated under reduced pressure. The crude material was purified by silica gel chromatography (0→50% EtOAc/hexanes) to provide the product (23.1 g, 59.2% yield) as a white solid. LCMS (ESI) m/z: [M+Na] calcd for C₅₈H₈₂N₂O₁₇: 1101.55; found 1101.6.

### Monomer 2. 32(R)-hydroxy 40(R)—O-(4-nitrophenyl)carbonate rapamycin

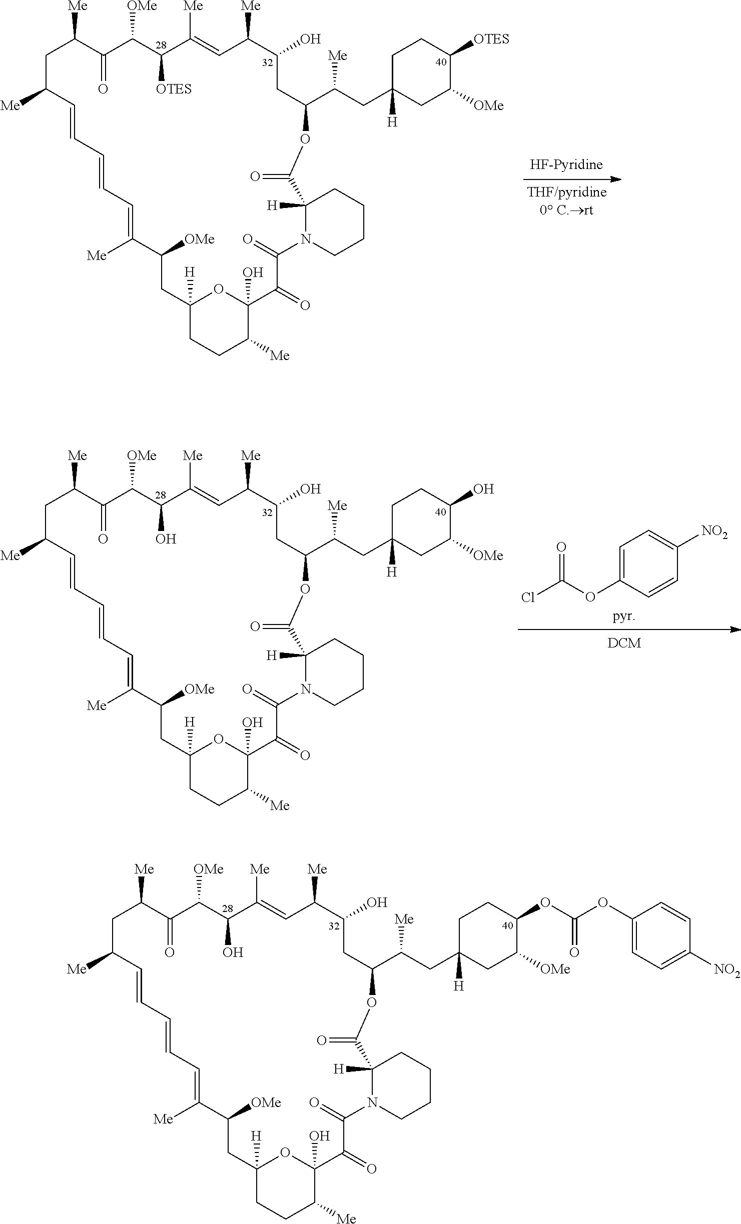

### Step 1: Synthesis of 32(R)-hydroxy rapamycin

A solution of 32(R)-hydroxy-28,40-bistriethylsilyl rapamycin (3.64 g, 3.18 mmol, 1 equiv) in THF (41.8 mL) was treated with pyridine (20.8 mL, 258 mmol, 81 equiv) and the reaction mixture was cooled to 0° C. The solution was treated dropwise with 70% HF-pyridine (4.60 mL, 159 mmol, 50 equiv) and the reaction mixture was stirred at 0° C. for 20 min followed by warming to room temperature. After 5 h, the reaction mixture was cooled back to 0° C. and carefully added to ice cold sat. NaHCO₃ solution (400 mL). The mixture was extracted with EtOAc (2×100 mL) and the organic phases were washed with 75 mL portions of H₂O, sat. NaHCO₃ solution and brine. The organic solution was dried over Na₂SO₄, filtered and concentrated to yield a light yellow oil that produced a stiff foam under reduced pressure. The crude material was purified by silica gel chromatography (20→40% acetone/hexanes) to yield the desired product as a white amorphous solid (1.66 g, 57% yield). LCMS (ESI) m/z: [M+Na] calcd for C₅₁H₈₁NO₁₃: 938.56; found 938.7; m/z: [M−H] calcd for C₅₁H₈₁NO₁₃: 914.56; found 914.7.

### Step 2: Synthesis of 32(R)-hydroxy 40(R)—O-(4-nitrophenyl)carbonate rapamycin

To a suspension of powdered 4 Å molecular sieves (6.0 g) in DCM (130 mL) was added 32(R)-hydroxy rapamycin (6.00 g, 6.55 mmol, 1.0 equiv). After stirring at room temperature for 45 min, pyridine (5.99 mL, 74.0 mmol, 11.3 equiv) was added. The suspension was cooled to −15° C. and then 4-nitrophenylchloroformate (1.78 g, 8.84 mmol, 1.4 equiv) was then added. The reaction mixture was stirred at −10° C. for 2 h and then filtered, and the filter pad washed with DCM (140 mL). The filtrate was washed with sat. NaHCO₃ (130 mL), H₂O (130 mL) and brine (130 mL), dried over Na₂SO₄, filtered and concentrated under reduced pressure. The crude material was purified by silica gel chromatography (20→50% EtOAc/hexanes) to give the product (4.44 g, 63% yield) as an off-white stiff foam. LCMS (ESI) m/z: [M+Na] calcd for C₅₈H₈₄N₂O₁₇: 1103.57; found 1103.5.

### Monomer 3. 32(R)-methoxy 40(R)—O-(4-nitrophenyl)carbonate rapamycin

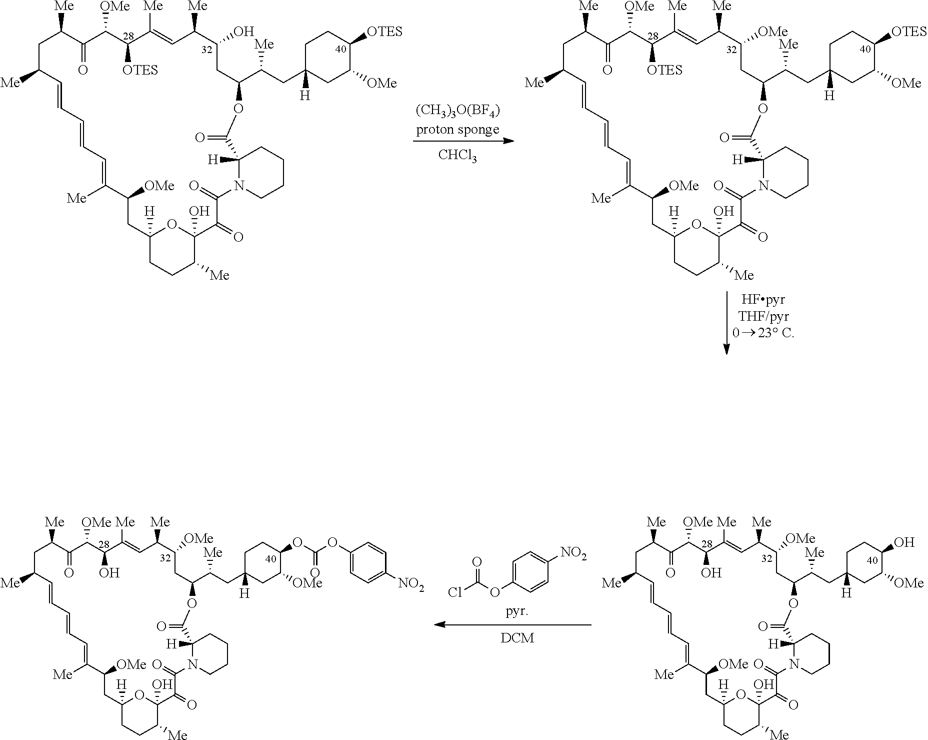

### Step 1: Synthesis of 32(R)-methoxy-28,40-bistriethylsilyl rapamycin

To a stirred solution of 32(R)-hydroxy-28,40-bistriethylsilyl rapamycin (3.83 g, 3.34 mmol, 1.0 equiv) in chloroform (95.8 mL) was added Proton Sponge® (7.17 g, 33.5 mmol, 10.0 equiv) along with freshly dried 4 Å molecular sieves (4 g). The solution was stirred for 1 h prior to the addition of trimethyloxonium tetrafluoroborate (4.95 g, 33.5 mmol, 10.0 equiv, dried by heating under reduced pressure at 50° C. for 1 h before use) at room temperature. The reaction mixture was stirred for 18 h, and then the reaction mixture was diluted with DCM and filtered through Celite. The filtrate was washed sequentially with aqueous 1 M HCl (2×), sat. aqueous NaHCO₃ solution, then dried and concentrated under reduced pressure. Purification by silica gel chromatography (10→20% EtOAc/hexanes) afforded the desired product as a yellow oil that was contaminated with 3 wt. % Proton Sponge®. The residue was taken up in MTBE and washed with aqueous 1 M HCl, sat. aqueous NaHCO₃ solution, dried, and then concentrated under reduced pressure to furnish a yellow foam (3.15 g, 81.2% yield). LCMS (ESI) m/z: [M−TES+H₂O] calcd for C₆₄H₁₁₁NO₁₃Si₂: 1061.68; found 1061.9.

### Step 2: Synthesis of 32(R)-methoxy rapamycin

To a stirred solution of 32(R)-methoxy-28,40-bistriethylsilyl rapamycin (1.11 g, 0.958 mmol, 1.0 equiv) in THF (12.6 mL) and pyridine (6.30 mL) in a plastic vial was added 70% HF-pyridine (2.22 mL, 76.6 mmol, 80.0 equiv) dropwise at 0° C. The reaction mixture was stirred at 0° C. for 20 min before being warmed to room temperature for 3 h, when HPLC showed complete consumption of starting material. The reaction mixture was cooled to 0° C. and poured slowly into ice cold sat. aqueous NaHCO₃ solution (50 mL). The aqueous layer was extracted with EtOAc (3×) and the combined organics were washed with sat. aqueous NaHCO₃ solution, brine, dried, and concentrated under reduced pressure. The yellow residue was dissolved in MeOH (5 mL) and added dropwise to H₂O (50 mL) to produce a white precipitate. After stirring for 15 min the slurry was filtered on a medium porosity funnel and the cake washed with H₂O (2×). The solids were then dissolved in MeCN (50 mL) and lyophilized overnight to provide the product as a white solid (780 mg, 87% yield). LCMS (ESI) m/z: [M+Na] calcd for C₅₂H₈₃NO₁₃: 952.58; found 952.4.

### Step 3: Synthesis of 32(R)-methoxy 40(R)—O-(4-nitrophenyl)carbonate rapamycin

To a solution of 32(R)-methoxy rapamycin (4.50 g, 4.84 mmol, 1.0 equiv) in DCM (180 mL) was added powdered 4 Å molecular sieves (6.0 g). The mixture was stirred at room temperature for 1 h and then pyridine (3.91 mL, 48.4 mmol, 10 equiv) was added. The mixture was cooled to −10° C. and 4-nitrophenylchloroformate (0.990 g, 4.91 mmol, 1.0 equiv) was added in one portion. The reaction was allowed to slowly warm to room temperature and after 3 h the reaction mixture was cooled to 0° C. and 4-nitrophenylchloroformate (250 mg, 1.24 mmol, 0.3 equiv) was added. The mixture was warmed to room temperature and after 1 h the reaction mixture was filtered through a pad of celite and the pad was washed with DCM (140 mL). The filtrate was washed with H₂O (120 mL) and sat NaHCO₃ (2×120 mL). The organic phase was dried over Na₂SO₄, filtered and concentrated under reduced pressure. The crude material was purified by flash chromatography (20→50% EtOAc/hex) to yield a white stiff foam. The material was taken up in MeCN during which time a white solid formed. The solid was filtered, washed with additional MeCN and allowed to air dry to provide the product (4.51 g, 85% yield). LCMS (ESI) m/z [M+Na] calcd for C₅₉H₈₆N₂O₁₇: 1117.58; found 1117.6.

### Monomers 4. 32(R)-ethoxy 40(R)—O-(4-nitrophenyl)carbonate rapamycin

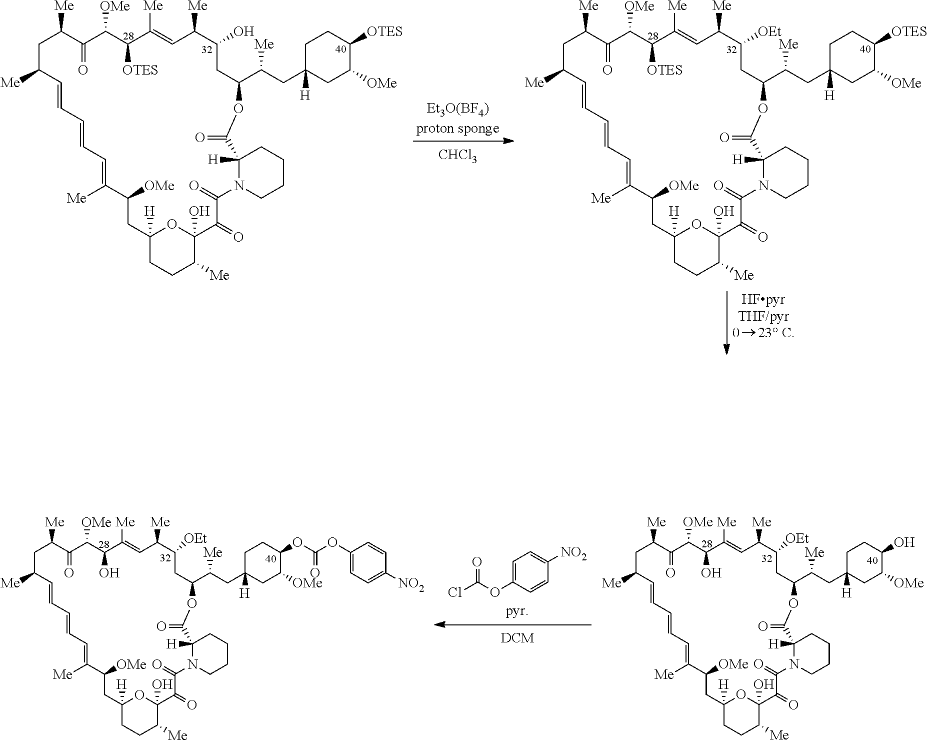

### Step 1: Synthesis of 32(R)-ethoxy-28,40-bistriethylsilyl rapamycin

A solution of 32(R)-hydroxy-28,40-bistriethylsilyl rapamycin (773 mg, 0.675 mmol, 1.0 equiv) in chloroform (19 mL) was treated with N,N,N′,N′-tetramethyl-1,8-naphthalenediamine (1.85 g, 8.63 mmol, 12.8 equiv) along with freshly dried 4× molecular sieves. The mixture was stirred for 1 h at room temperature and treated with triethyloxonium tetrafluoroborate (1.51 g, 7.95 mmol, 11.8 equiv) in one portion at room temperature. The reaction mixture was stirred for 3 h, at which point the reaction mixture was diluted with DCM and filtered through Celite, washing the filter pad with additional DCM. The combined filtrates were washed twice with 1 M HCl, once with saturated NaHCO₃ solution, and dried over Na₂SO₄. The solution was filtered and concentrated to a residue. The crude residue was treated with MTBE and filtered to remove polar insoluble material. The filtrate was concentrated and purified by silica gel chromatography (5→25% EtOAc/hex) to afford the product as a foam (516 mg, 65% yield). LCMS (ESI) m/z: [M+Na] calcd for C₆₅H₁₁₃NO₁₃Si₂ 1194.77; found 1194.6.

### Step 2: Synthesis of 32(R)-ethoxy rapamycin

To a solution of 32(R)-ethoxyethoxy-28,40-bistriethylsilyl rapamycin (131 mg, 0.112 mmol, 1.0 equiv) in THF (1.3 mL) at 0° C. was added pyridine (271 μL, 3.35 mmol, 3.4 equiv) followed by 70% HF-pyridine (51 μL, 1.8 mmol, 1.8 equiv). The reaction flask was capped and stored in the fridge for 3 days, at which point the reaction mixture was poured into cold saturated NaHCO₃ (20 mL). The aqueous layer extracted with EtOAc (3×20 mL) and the combined organic layers were washed with 1 M HCl (2×20 mL), saturated NaHCO₃ solution (20 mL), and brine. The solution was dried over Na₂SO₄, filtered, and concentrated. The residue was taken up in MeOH (1.5 mL) and added dropwise to H₂O (20 mL). The solids were filtered and washed with additional H₂O to provide the product (53 mg, 51% yield) as a white powder. LCMS (ESI) m/z: [M+Na] calcd for C₅₃H₈₅NO₁₃: 966.59; found 966.5.

### Step 3: Synthesis of 32(R)-ethoxy 40(R)—O-(4-nitrophenyl)carbonate rapamycin

To a 0.03 M solution of 32(R)-ethoxy rapamycin (1.0 equiv) in DCM is added powdered 4 Å molecular sieves. The mixture is stirred at room temperature for 1 h and then pyridine (10 equiv) is added. The mixture is cooled to −10° C. and 4-nitrophenylchloroformate (1.0 equiv) is added in one portion. The reaction is warmed to room temperature and stirred until consumption of 32(R)-ethoxy rapamycin, as determined by LCMS analysis. The mixture is filtered through a pad of celite and the pad washed with DCM. The filtrate is washed with H₂O and sat NaHCO₃. The organic phase is then dried over Na₂SO₄, filtered and concentrated under reduced pressure. The crude material is purified by flash chromatography (20→50% EtOAc/hex) to provide the product.

### Monomer 5. 40(R)—O-(4-nitrophenyl)thiocarbonate rapamycin

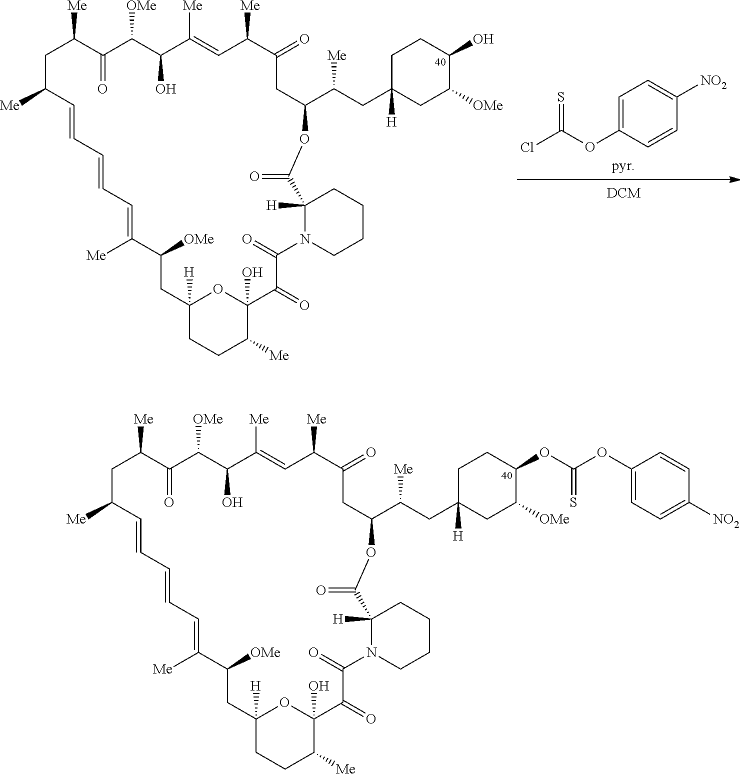

To a solution of rapamycin (4.00 g, 4.38 mmol, 1.0 equiv) in DCM (20 mL) at −78° C. was added pyridine (4.0 mL, 49 mmol, 11.2 equiv), followed by a solution of O-(4-nitrophenyl)chlorothiocarbonate (1.19 g, 5.47 mmol, 1.3 equiv) in DCM (8.0 mL). The reaction mixture was warmed to −20° C. and stirred for 48 h. Hexane (40 mL) was then added and the resulting suspension was purified by silica gel chromatography (15/25/60 EtOAc/DCM/hexane then 20/25/55 EtOAc/DCM/hexane) to provide the product (3.09 g, 64.4% yield) as an off-white solid. LCMS (ESI) m/z: [M+Na] calcd for C₅₈H₈₂N₂O₁₆S: 1117.53; found 1117.5.

### Monomer 6. 28-O-(4-nitrophenyl)carbonate rapamycin

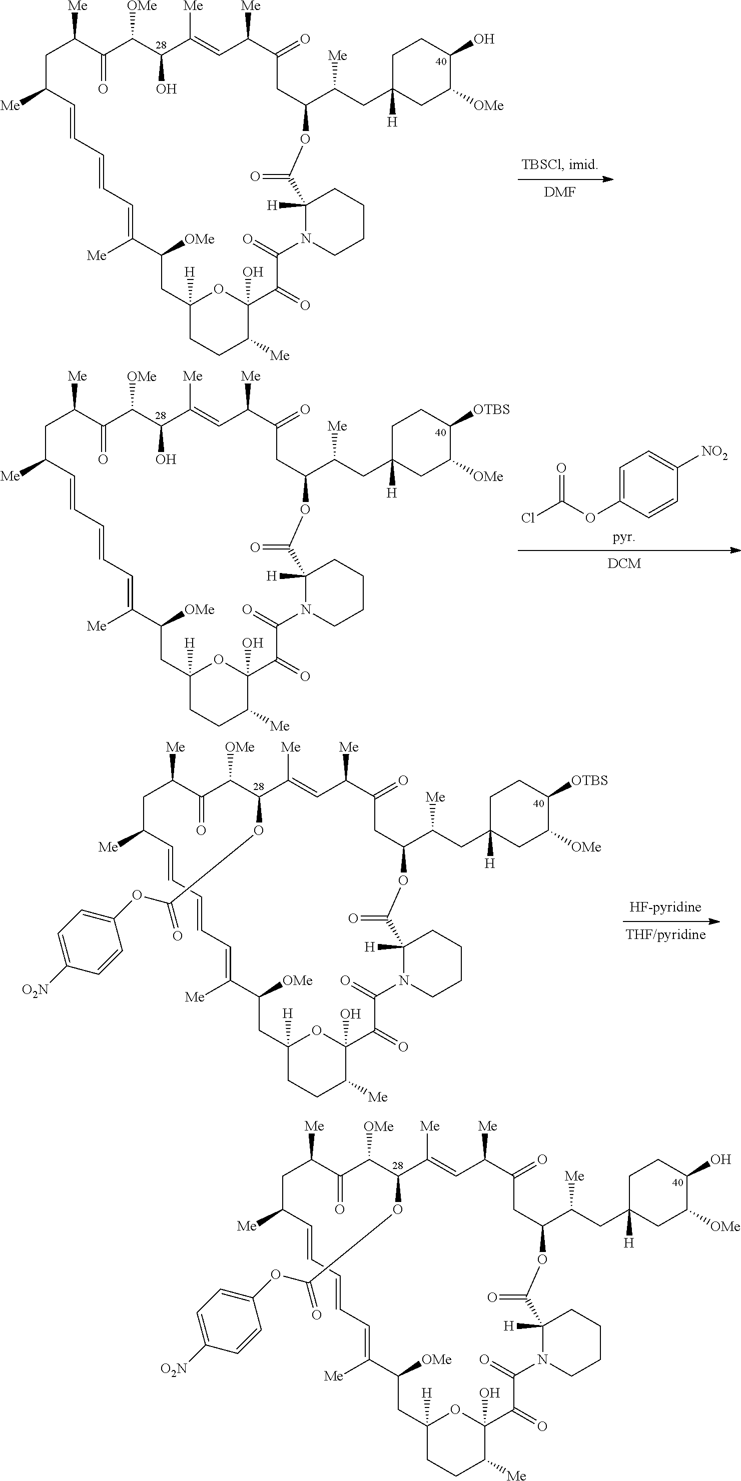

### Step 1: Synthesis of 40-O-tert-butyldimethyl silyl rapamycin

To a solution of rapamycin (1.00 g, 1.09 mmol, 1.0 equiv) in DMF (4 mL) at room temperature was added imidazole (0.22 g, 3.2 mmol, 2.9 equiv) followed by tert-butyldimethylsilyl chloride (0.176 g, 1.17 mmol, 1.07 equiv). The reaction mixture was stirred for 18 h. The reaction mixture was then diluted with DCM (100 mL) and washed with 20% aq. LiCl (3×20 mL). The organic layer was dried over Na₂SO₄, filtered and concentrated under reduced pressure. The residue was purified by silica gel chromatography (20→40% EtOAc/hexanes) to give the product (950 mg, 75% yield) as a faint yellow glass. LCMS (ESI) m/z: [M+H₂O] calcd for C₅₇H₉₃NO₁₃Si: 1045.65; found 1045.9.

### Step 2: Synthesis of 28-O-(4-nitrophenoxycarbonyl)-40-O-(tert-butyldimethylsilyl) rapamycin

To a solution of 40-O-tert-butyldimethylsilyl rapamycin (0.845 g, 0.822 mmol, 1.0 equiv) in DCM (10 mL) at room temperature was added pyridine (0.9 mL, 10 mmol, 12.1 equiv) followed by 4-nitrophenyl chloroformate (0.373 g, 1.85 mmol, 2.25 equiv). The reaction mixture was stirred for 2 h. The reaction mixture was then diluted with DCM (150 mL) and the solution sequentially washed with sat. NaHCO₃ (20 mL), 10% citric acid (2×20 mL), and brine (20 mL). The organic layer was dried over Na₂SO₄, filtered, and concentrated under reduced pressure. The residue was purified by reverse phase chromatography (30→100% MeCN/H₂O) to give the product (930 mg, 95% yield) as a pale yellow foam. LCMS (ESI) m/z: [M+H] calcd for C₆₄H₉₆N₂O₁₇Si: 1193.66; found 1193.7.

### Step 3: Synthesis of 28-O-(4-nitrophenoxycarbonyl) rapamycin

To a solution of 28-O-(4-nitrophenoxycarbonyl)-40-O-(tert-butyldimethylsilyl)rapamycin (0.930 g, 0.779 mmol, 1.0 equiv) in THF (10.7 mL) was added pyridine (3.78 mL, 46.8 mmol, 60.1 equiv) followed by the dropwise addition of 70% HF-pyridine (0.91 mL, 31.2 mmol, 40.0 equiv). The reaction mixture was stirred at room temperature for 48 h. The mixture was then poured slowly into ice cold sat. aqueous NaHCO₃ (20 mL). The aqueous layer was extracted with EtOAc (3×20 mL) and the combined organic layer was washed with sat. NaHCO₃ (10 mL) and brine (10 mL), dried over Na₂SO₄, filtered, and concentrated under reduced pressure. The residue was purified by reverse phase chromatography (30→100% MeCN/H₂O) to give the product (200 mg, 24% yield) as a faint yellow powder. LCMS (ESI) m/z: [M+Na] calcd for C₅₈H₈₂N₂O₁₇: 1101.55; found 1101.3.

### Monomer 7. 32(R)-hydroxy 40(R)—O-(4-nitrophenyl)thiocarbonate rapamycin

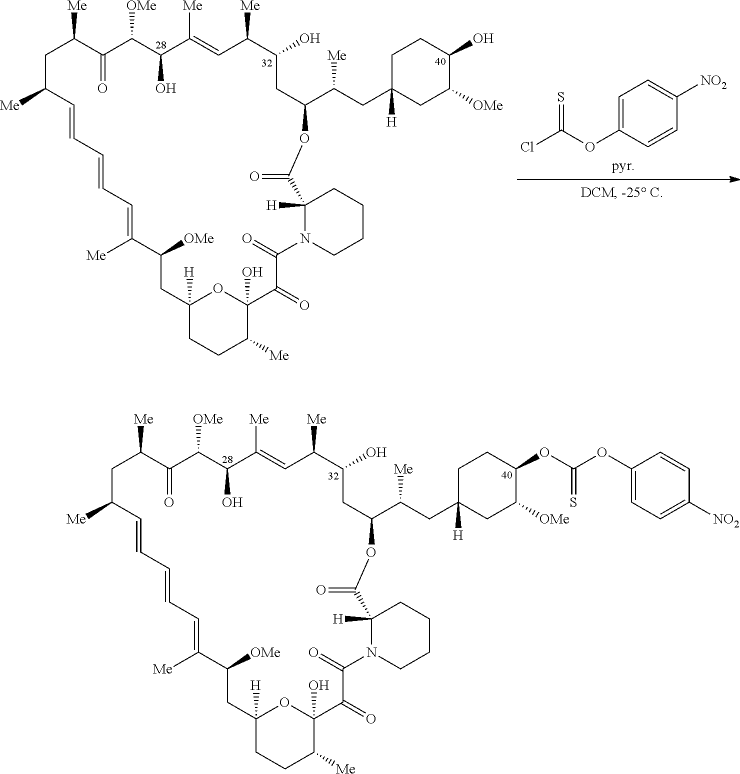

To a solution of 32(R)-hydroxy rapamycin (2.80 g, 3.06 mmol, 1.0 equiv) in DCM (28 mL) was added pyridine (27.6 mL, 34 mmol, 11 equiv) and dried 4 Å molecular sieves (2.8 g). The suspension was stirred at room temperature for 1 h, at which point the mixture was cooled to −25° C. and a solution of O-(4-nitrophenyl)chlorothioformate (0.798 g, 3.67 mmol, 1.2 equiv) in DCM (6 mL) was added. The reaction was warmed to room temperature and after 21 h was filtered through Celite. The filtrate was partitioned between DCM and H₂O and the aqueous layer was extracted with DCM. The combined organic layers were dried and concentrated under reduced pressure. Purification by silica gel chromatography (EtOAc/hexanes) afforded the desired product as a white solid (2.15 g, 64% yield). LCMS (ESI) m/z: [M+Na] calcd for C₅₈H₈₄N₂O₁₆S: 1119.54; found 1120.0.

### Monomer 8. 32(R)-methoxy 40(R)—O-(4-nitrophenyl)thiocarbonate rapamycin

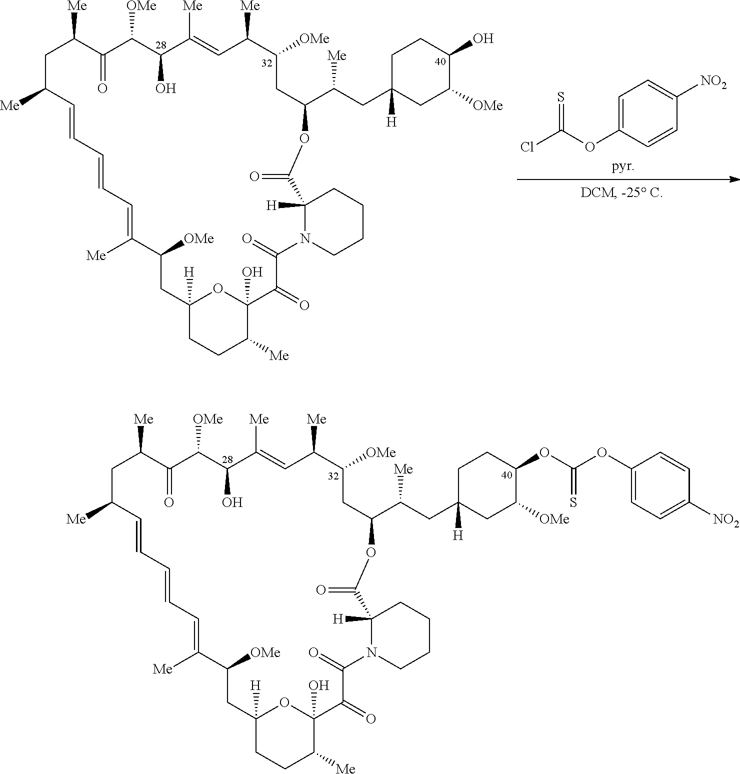

To a solution of 32(R)-methoxy rapamycin (6.69 g, 7.19 mmol, 1.0 equiv) in DCM (67 mL) was added pyridine (6.6 mL, 81 mmol, 11 equiv) and dried 4 Å molecular sieves (6.7 g). The suspension was stirred at room temperature for 1 h, at which point the mixture was cooled to −25° C. and a solution of O-(4-nitrophenyl)chlorothioformate (1.88 g, 8.63 mmol, 1.20 equiv) in DCM (13 mL) was added. The reaction was warmed to room temperature and after 21 h was filtered through Celite. The filtrate was partitioned between DCM and H₂O and the aqueous layer was extracted with DCM. The combined organic layers were dried and concentrated under reduced pressure. Purification by silica gel chromatography (EtOAc/hexanes) afforded the desired product as a white solid (5.1 g, 64% yield). LCMS (ESI) m/z: [M+Na] calcd for C₉H₈₆N₂O₁₆S: 1133.56; found 1134.1.

### Monomer 9. 32-deoxy 40(R)—O-(4-nitrophenyl)carbonate rapamycin

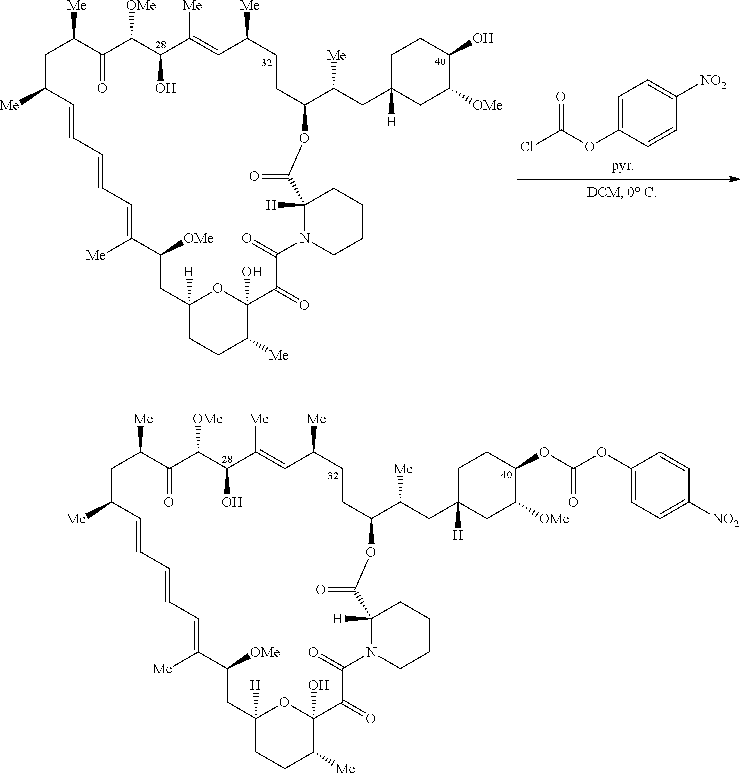

To a solution of 32-deoxy rapamycin (0.623 g, 0.692 mmol, 1.0 equiv) in DCM (24.7 mL) was added 4 Å molecular sieves (600 mg). The suspension was stirred for 1 h and then pyridine (557 μL, 6.92 mmol, 10 equiv) was added. The reaction mixture was cooled to 0° C. and then O-(4-nitrophenyl)chloroformate (175 mg, 1.03 mmol, 1.7 equiv) was added. The reaction warmed to room temperature and stirred for 2 h, at which point the reaction mixture was concentrated under reduced pressure. Purification by silica gel chromatography (0→10% MeOH/DCM) afforded the desired product as a white solid (0.61 g, 82% yield). LCMS (ESI) m/z: [M+Na] calcd for C₅₈H₈₄N₂O₁₆: 1087.57; found 1087.6.

### Monomer 10. 32-deoxy 40(R)—O-(4-nitrophenyl)thiocarbonate rapamycin

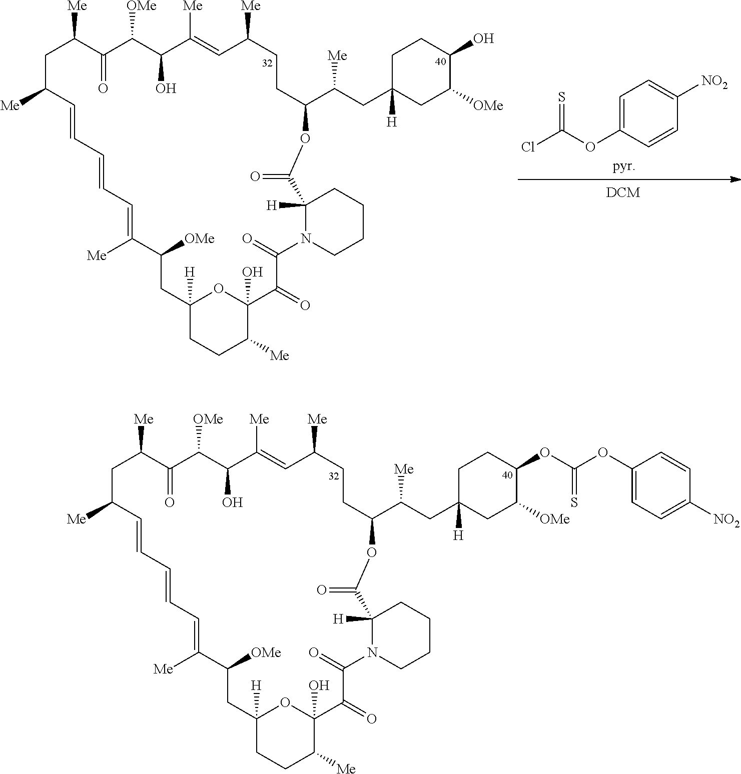

To a 0.2 M solution of 32-deoxy rapamycin (1.0 equiv) in DCM at −78° C. is added pyridine (11.2 equiv), followed by a 0.7 M solution of O-(4-nitrophenyl)chlorothiocarbonate (1.3 equiv) in DCM. The reaction mixture is warmed to −20° C. and stirred until consumption of the starting material, as determined by LCMS analysis. Hexane is then added and the resulting suspension is purified by silica gel chromatography to provide the product.

### Monomer 11. 28(R)-methoxy 32(R)-hydroxy 40(R)-(4-nitrophenyl)carbonate rapamycin

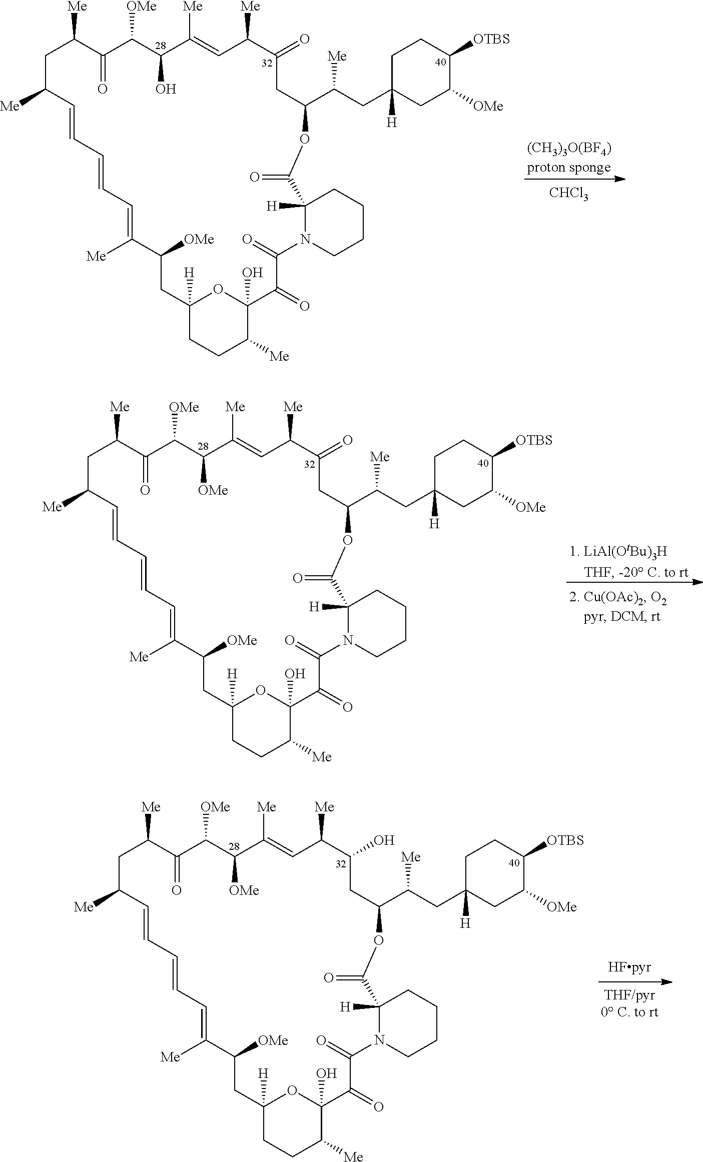

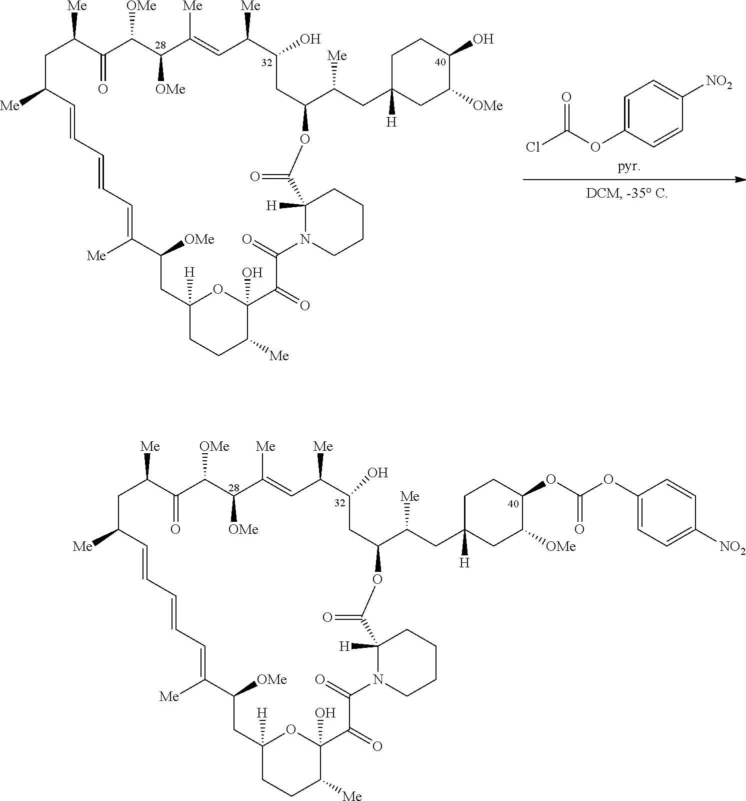

### Step 1: Synthesis of 28(R)-methoxy 40(R)—O-tert-butyldimethylsilyl rapamycin

To a solution of 40(R)—O-tert-butyldimethylsilyl rapamycin (4.00 g, 4.89 mmol, 1.0 equiv) in chloroform (67 mL) was added proton sponge (11.2 mL, 52.3 mmol, 13 equiv) and dried 4 Å molecular sieves (5.8 g). The suspension was stirred at room temperature for 1 h, at which point trimethyloxonium tetrafluoroborate (7.21 g, 48.8 mmol, 12.5 equiv) was added. After 4 h the suspension was filtered through Celite. The filtrate was washed sequentially with aqueous 2 N HCl, H₂O, sat. aqueous NaHCO₃, then dried and concentrated under reduced pressure. Purification by silica gel chromatography (EtOAc/hexane) afforded the desired product as a white solid (2.1 g, 52% yield). LCMS (ESI) m/z: [M+Na] calcd for C₅₈H₉₅NO₁₃Si: 1064.65; found 1065.26.

### Step 2: Synthesis of 28(R)-methoxy 32(R)-hydroxy 40(R)—O-tert-butyldimethylsilyl rapamycin

To a solution of 28(R)-methoxy 40(R)—O-tert-butyldimethylsilyl rapamycin (2.13 g, 2.04 mmol, 1.0 equiv) in THF (31 mL) at −20° C. was added a solution of lithium tri-tert-butoxyaluminum hydride in THF (1 M, 4.09 mL, 4.09 mmol, 2.0 equiv), dropwise. The reaction mixture was warmed to room temperature and after 3 h was added to a solution of H₂O (4 mL), EtOAc (31 mL), and 2M aqueous citric acid (4 mL) at 0° C. After 5 min the mixture was partitioned, and the aqueous layer was extracted with EtOAc. The combined organic layers were poured into a sat. aqueous NaHCO₃ solution (60 mL) at 0° C. The layers were partitioned, and the organic layer was dried and concentrated under reduced pressure to provide a crude white solid (2.32 g). The crude solid was dissolved in DCM (12 mL) and then pyridine (241 μL, 2.98 mmol, 1.5 equiv), dried 4 Å molecular sieves (2.1 g), and cupric acetate (0.27 g, 1.49 mmol, 0.7 equiv) were added. The suspension was stirred at room temperature for 1 h. The suspension was sparged with 02 and then kept under an 02 atmosphere for 30 min. After 2 h the mixture was filtered through Celite and the filtrate was concentrated under reduced pressure. Purification by silica gel chromatography (EtOAc/hexane) afforded the desired product as a white solid (307 mg, 14% yield). LCMS (ESI) m/z: [M+Na] calcd for C₅₈H₉₇NO₁₃Si: 1066.66; found 1067.0.

### Step 3: Synthesis of 28(R)-methoxy 32(R)-hydroxy rapamycin

To a solution of 28(R)-methoxy 32(R)-hydroxy 40(R)—O-tert-butyldimethylsilyl rapamycin (0.307 g, 0.294 mmol, 1.0 equiv) in THF (4 mL) in a polypropylene vial at 0° C. was added pyridine (1.42 mL, 17.6 mmol, 60.0 equiv) followed by 70% HF-pyridine (0.34 mL, 11.7 mmol, 40 equiv). The solution was warmed to room temperature and stirred for 21 h, at which point the solution was poured into sat. aqueous NaHCO₃ at 0° C. The aqueous layer was extracted with EtOAc (2×) and the combined organic layers were washed with sat. aqueous NaHCO₃ and brine, then dried and concentrated under reduced pressure. Purification by silica gel chromatography (EtOAc/hexane) afforded the desired product as a white solid (146 mg, 53% yield). LCMS (ESI) m/z: [M+Na] calcd for C₅₂H₈₃NO₁₃: 952.58; found 952.8.

### Step 4: Synthesis of 28(R)-methoxy 32(R)-hydroxy 40(R)-(4-nitrophenyl)carbonate rapamycin

To a solution of 28(R)-methoxy 32(R)-hydroxy rapamycin (0.66 g, 0.71 mmol, 1.0 equiv) in DCM (3 mL) was added pyridine (0.64 mL, 7.9 mmol, 11 equiv) and dried 4 Å molecular sieves (0.66 g). The suspension was stirred at room temperature for 1 h, at which point the mixture was cooled to −35° C. and O-(4-nitrophenyl)chloroformate (0.17 g, 0.85 mmol, 1.2 equiv) was added. After 3 h, DCM (5 mL) was added and the suspension was filtered through Celite. The filtrate was washed with H₂O, dried, and concentrated under reduced pressure. Purification by silica gel chromatography (EtOAc/hexanes) afforded the desired product as a white solid (0.44 g, 57% yield). LCMS (ESI) m/z: [M+Na] calcd for C₅₉H₈₆N₂O₁₇: 1117.58; found 1118.0.

### Monomer 12. 28(R)-methoxy 32(R)-methoxy 40(R)-(4-nitrophenyl)carbonate rapamycin

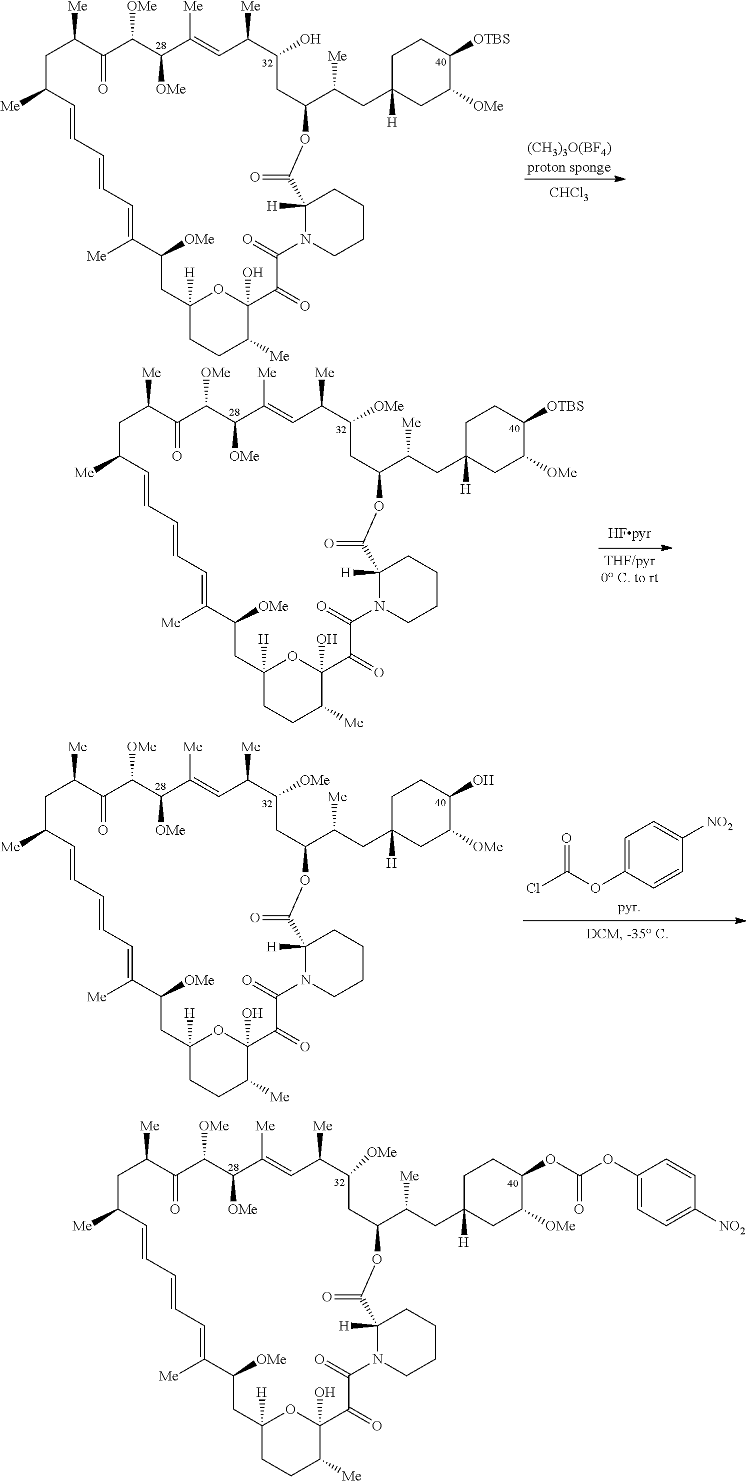

### Step 1: Synthesis of 28(R)-methoxy 32(R)-methoxy 40(R)—O-tert-butyldimethylsilyl rapamycin

To a solution of 28(R)-methoxy 32(R)-hydroxy 40(R)—O-tert-butyldimethylsilyl rapamycin (1.15 g, 1.10 mmol, 1.0 equiv) in chloroform (19 mL) was added proton sponge (3.22 mL, 15.0 mmol, 14 equiv) and dried 4 Å molecular sieves (2.3 g). The suspension was stirred at room temperature for 1 h, at which point trimethyloxonium tetrafluoroborate (2.07 g, 14.0 mmol, 12.7 equiv) was added. After 4 h the suspension was filtered through Celite and the filtrate was washed with 1N HCl, H₂O, and sat. aqueous NaHCO₃, dried and concentrated under reduced pressure. Purification by silica gel chromatography (EtOAc/hexane) afforded the desired product as a white solid. LCMS (ESI) m/z: [M+Na] calcd for C₅₉H₉₉NO₁₃Si: 1080.68; found 1081.2.

### Step 2: Synthesis of 28(R)-methoxy 32(R)-methoxy rapamycin

To a solution of 28(R)-methoxy 32(R)-methoxy 40(R)—O-tert-butyldimethylsilyl rapamycin in THF (4 mL) in a polypropylene vial at 0° C. was added pyridine (1.13 mL, 14.2 mmol, 12.9 equiv) followed by 70% HF-pyridine (0.27 mL, 9.42 mmol, 8.6 equiv). The solution was warmed to room temperature and stirred for 41 h, at which point the solution was poured into sat. aqueous NaHCO₃ at 0° C. The aqueous layer was extracted with EtOAc (2×) and the combined organic layers were washed with sat. aqueous NaHCO₃ and brine, then dried and concentrated under reduced pressure. Purification by silica gel chromatography (EtOAc/hexane) afforded the desired product as a white solid (516 mg, 49% yield 2-steps). LCMS (ESI) m/z: [M+Na] calcd for C₅₃H₈₅NO₁₃: 966.59; found 967.0.

### Step 3: Synthesis of 28(R)-methoxy 32(R)-methoxy 40(R)-(4-nitrophenyl)carbonate rapamycin

To a solution of 28(R)-methoxy 32(R)-methoxy rapamycin (0.30 g, 0.32 mmol, 1.0 equiv) in DCM (1.4 mL) was added pyridine (0.29 mL, 3.5 mmol, 11 equiv) and dried 4 Å molecular sieves (0.30 g). The suspension was stirred at room temperature for 1 h, at which point it was cooled to −35° C. and O-(4-nitrophenyl)chloroformate (0.08 g, 0.38 mmol, 1.2 equiv) was added. After 3 h, DCM (2 mL) was added and the suspension was filtered through Celite. The filtrate was washed with H₂O, dried and concentrated under reduced pressure. Purification by silica gel chromatography (EtOAc/hexanes) afforded the desired product as an off-white solid (0.20 g, 57% yield). LCMS (ESI) m/z: [M+Na] calcd for C₆₀H₈₈N₂O₁₇: 1131.60; found 1132.1.

### Monomer 13. 32(R)-(4-nitrophenyl)carbonate rapamycin

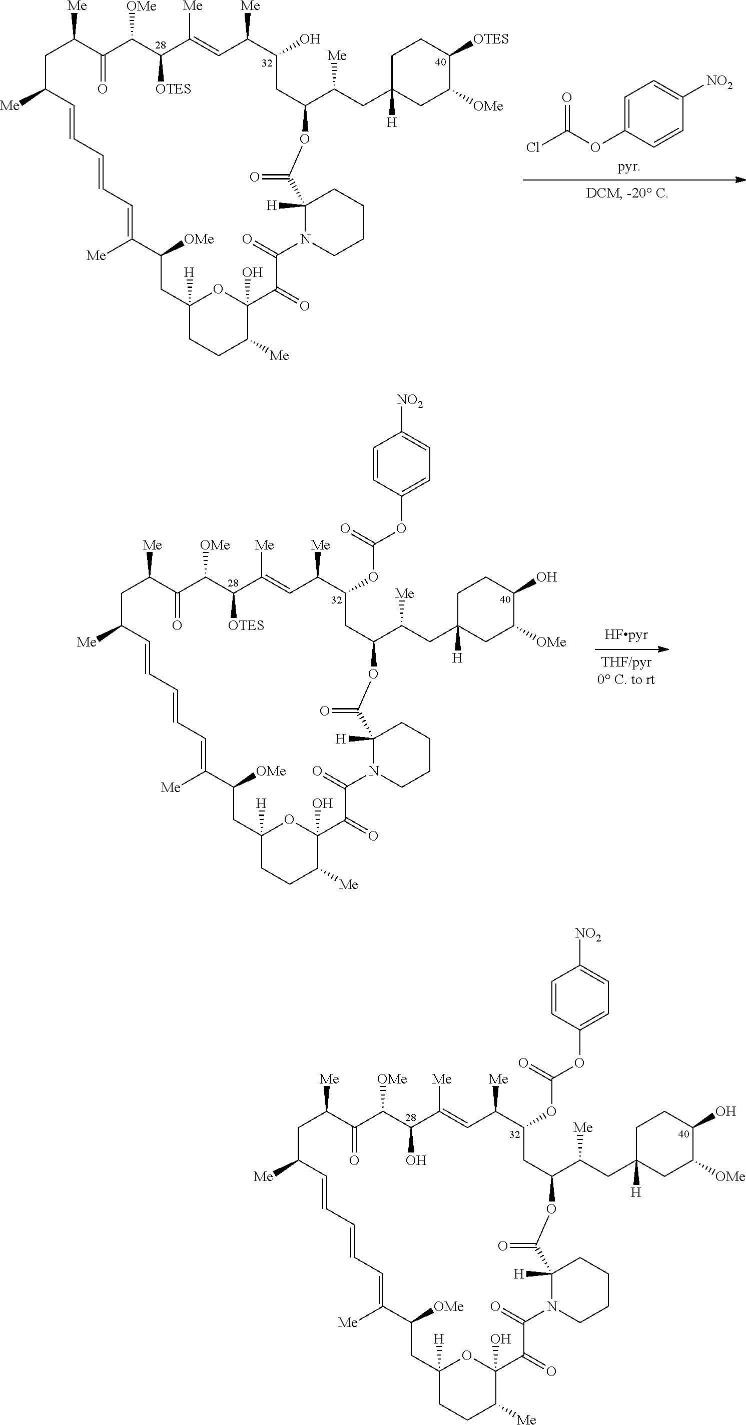

### Step 1: Synthesis of 28,40-O-bis(triethylsilyl) 32(R)-(4-nitrophenyl)carbonate rapamycin

To a solution of 28,40-O-bis(triethylsilyl) 32(R)-hydroxy rapamycin (0.602 g, 0.526 mmol, 1.0 equiv) in DCM (16 mL) at −20° C. was added pyridine (0.82 mL, 10 mmol, 19 equiv) followed by O-(4-nitrophenyl)chloroformate (0.36 g, 1.8 mmol, 3.4 equiv). The reaction mixture was warmed to room temperature and stirred for 1 h, at which point the solution was diluted with DCM (50 mL) and poured into H₂O (30 mL). The aqueous layer was extracted with DCM (50 mL) and the combined organic layers were washed with brine (20 mL), dried and concentrated under reduced pressure to afford a faint yellow foam that was used directly in the next step.

### Step 2: Synthesis of 32(R)-(4-nitrophenyl)carbonate rapamycin

To a solution of 28,40-O-bis(triethylsilyl) 32(R)-(4-nitrophenyl)carbonate rapamycin in THF (10 mL) in a polypropylene vial at 0° C. was added pyridine (1.70 mL, 21.0 mmol, 40.0 equiv) followed by 70% HF-pyridine (0.46 mL, 15.8 mmol, 30.0 equiv). The solution was warmed to room temperature and stirred overnight, at which point the solution was poured into sat. aqueous NaHCO₃ at 0° C. The aqueous layer was extracted with EtOAc (3×) and the combined organic layers were washed with sat. aqueous NaHCO₃ and brine, then dried and concentrated under reduced pressure. Purification by reverse phase chromatography (20→100% MeCN/H₂O) afforded the desired product as an off-white powder (420 mg, 74% yield 2-steps). LCMS (ESI) m/z: [M+Na] calcd for C₅₈H₈₄N₂O₁₇: 1103.57; found 1104.0.

### Monomer 14. 32(S)-azido 40-(4-nitrophenyl)carbonate rapamycin

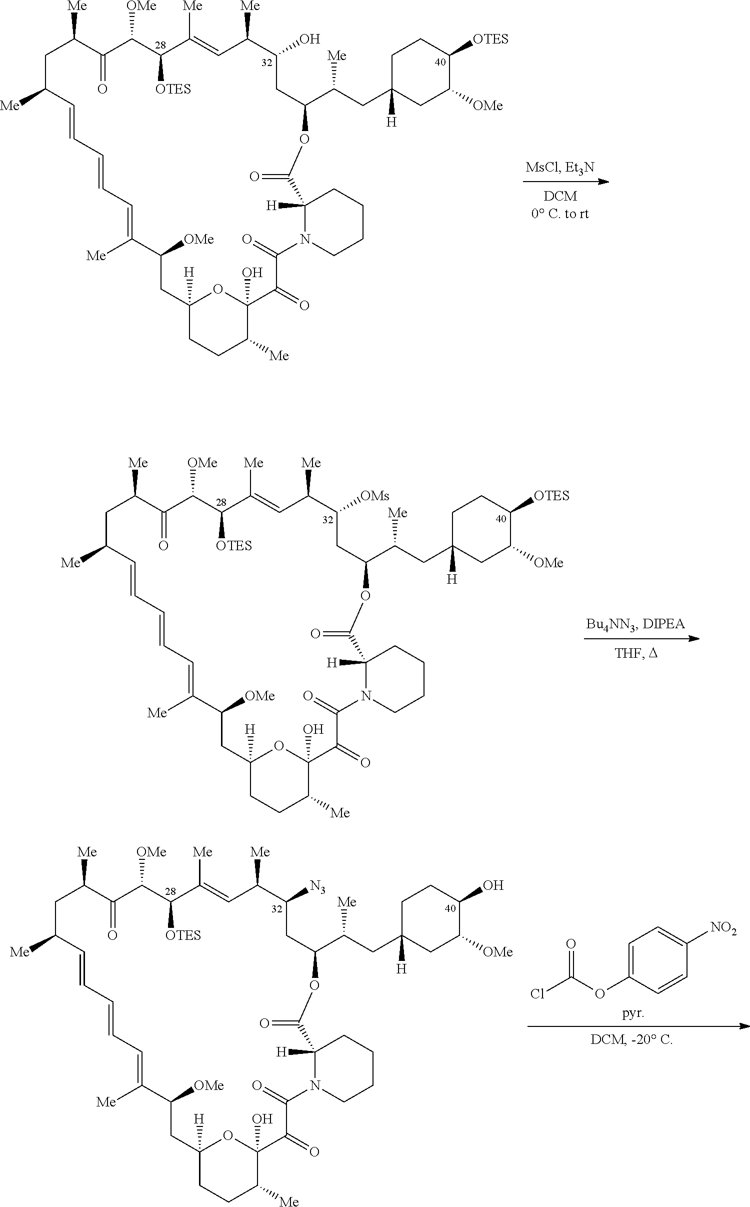

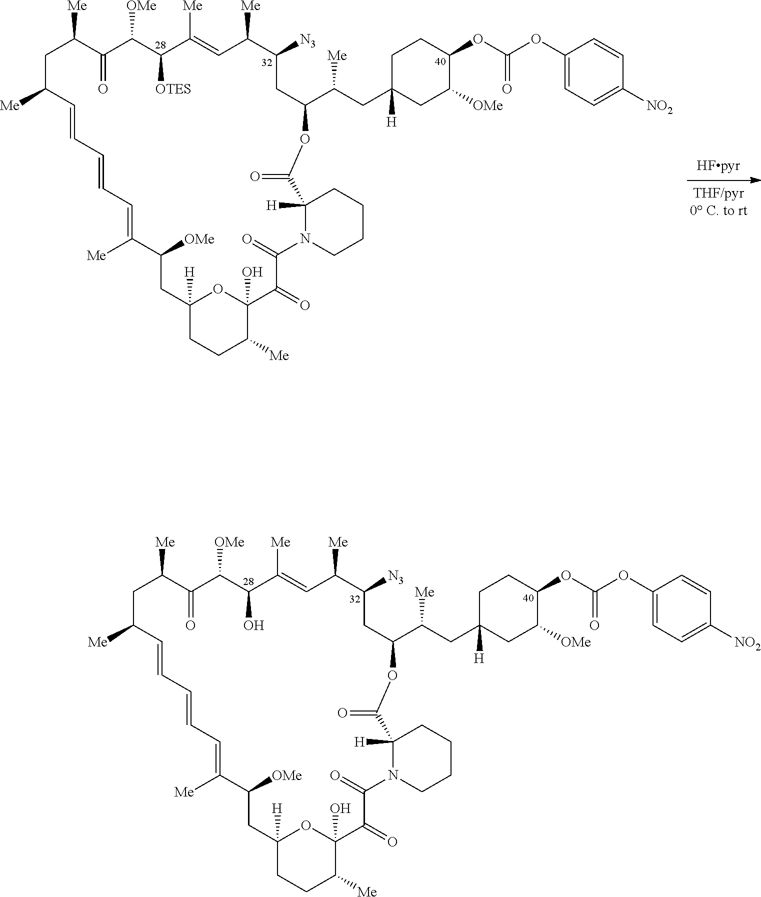

### Step 1: Synthesis of 28,40-O-bis(triethylsilyl) 32(R)—O-methanesulfonyl rapamycin

To a solution of 28,40-O-bis(triethylsilyl) 32(R)-hydroxy rapamycin (2.50 g, 2.18 mmol, 1.0 equiv) in DCM (25 mL) at 0° C. was added Et₃N (0.912 mL, 6.54 mmol, 3.0 equiv) followed by methanesulfonyl chloride (0.338 mL, 4.36 mmol, 2.0 equiv). The solution was stirred at 0° C. for 3 h, at which point the EtOAc was added and the solution was washed with sat. aqueous NaHCO₃. The combined organic layers were washed with brine, dried and concentrated under reduced pressure to give a yellow oil which was used directly in the next step.

### Step 2: Synthesis of 28-O-triethylsilyl 32(S)-azido rapamycin

To a solution of 28,40-O-bis(triethylsilyl) 32(R)—O-methanesulfonyl rapamycin in THF (40 mL) was added DIPEA (0.761 mL, 4.37 mmol, 2.0 equiv) and tetrabutylammonium azide (3.72 g, 13.1 mmol, 6.0 equiv). The reaction solution heated to reflux for 5.5 h and then cooled to room temperature. The solution was diluted with EtOAc and washed with sat. aqueous NaHCO₃. The combined organic layers were washed with brine, dried and concentrated under reduced pressure. Purification by reverse phase chromatography (30→100% MeCN/H₂O) afforded the desired product as a clear glass (746 mg, 33% yield 2-steps). LCMS (ESI) m/z: [M+Na] calcd for C₅₇H₉₄N₄O₁₂Si: 1077.65; found 1077.8.

### Step 3: Synthesis of 28-O-triethylsilyl 32(S)-azido 40(R)-(4-nitrophenyl)carbonate rapamycin

To a solution of 28-O-triethylsilyl 32(S)-azido rapamycin (0.505 g, 0.478 mmol, 1.0 equiv) in DCM (15 mL) was added pyridine (0.75 mL, 9.3 mmol, 19 equiv) and 4 Å molecular sieves. The suspension was cooled to −20° C. and O-(4-nitrophenyl)chloroformate (0.32 g, 1.6 mmol, 3.4 equiv) was added. The suspension was stirred at −20° C. for 2 h, at which point the it was diluted with DCM (50 mL), filtered and poured into H₂O (20 mL). The aqueous layer was extracted with DCM (50 mL) and the combined organic layers were washed with brine (20 mL), dried and concentrated under reduced pressure to give a pale-yellow foam which was used directly in the next step.

### Step 4: Synthesis of 32(S)-azido 40-O-(4-nitrophenyl)carbonate rapamycin

To a solution of 28-O-triethylsilyl 32(S)-azido 40(R)-(4-nitrophenyl)carbonate rapamycin in THF (10 mL) in a polypropylene vial at 0° C. was added pyridine (1.55 mL, 19.1 mmol, 40.0 equiv) followed by 70% HF-pyridine (0.42 mL, 14.4 mmol; 30.0 equiv). The solution was warmed to room temperature and stirred overnight, at which point the solution was poured into sat. aqueous NaHCO₃ at 0° C. The aqueous layer was extracted with EtOAc (3×) and the combined organic layers were washed with sat. aqueous NaHCO₃ and brine, then dried and concentrated under reduced pressure. Purification by reverse phase chromatography (30→100% MeCN/H₂O) afforded the desired product as an off-white powder (410 mg, 77% yield 2-steps). LCMS (ESI) m/z: [M+Na] calcd for C₅₈H₈₃N₅O₁₆: 1128.57; found 1129.0.

### Monomer 15. 28-methoxy-40-O-(4-nitrophenoxy)carbonyl rapamycin

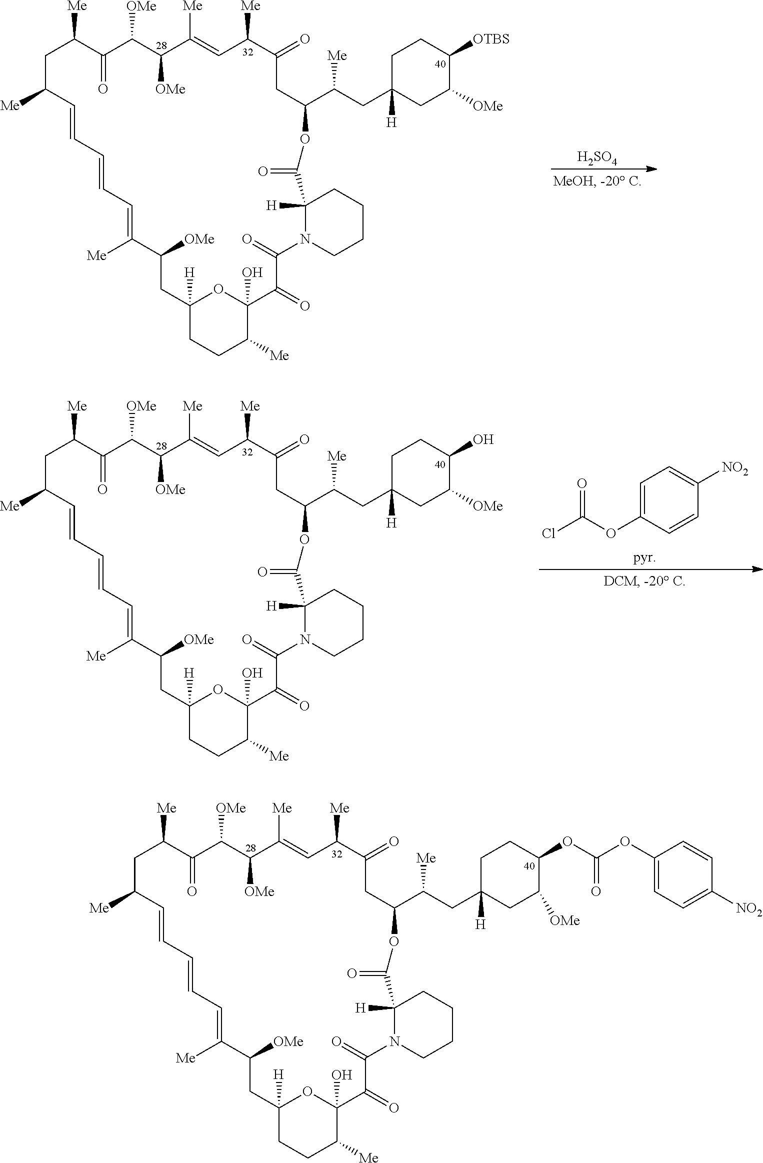

### Step 1: Synthesis of 28-methoxy rapamycin

To a solution of 28-methoxy-40-O-(tert-butyldimethyl)silyl rapamycin (0.500 g, 0.480 mmol, 1.0 equiv) in MeOH (1.6 mL) at −20° C. was added H₂SO₄ (1.28 μL, 0.024 mmol, 0.05 equiv). The reaction mixture was stirred at −20° C. for 48 h. The reaction mixture was then poured into sat. aqueous NaHCO₃ (4 mL)/H₂O (4 mL). The aqueous layer was extracted with MTBE (2×6 mL), and the combined organic phases were dried, filtered, and concentrated under reduced pressure. Purification by reverse phase chromatography (30→100% MeCN/H₂O) afforded the desired product as a yellow powder (270 mg, 61% yield). LCMS (ESI) m/z: [M+Na] calcd for C₅₂H₈₁NO₁₃: 950.5; found 950.7.

### Step 2: Synthesis of 28-methoxy-40-O-(4-nitrophenoxy)carbonyl rapamycin

To a solution of 28-methoxy rapamycin (0.210 g, 0.226 mmol, 1.0 equiv) in DCM (7.1 mL) at −20° C. was added pyridine (0.35 mL, 4.4 mmol, 19 equiv) and then p-nitrophenyl chloroformate (0.15 g, 0.76 mmol, 3.4 equiv). The reaction mixture was stirred at −20° C. for 30 min and then warmed to room temperature. After stirring overnight, p-nitrophenyl chloroformate (0.15 g, 0.76 mmol, 3.4 equiv) was added and the reaction was stirred for an additional 2 h. The reaction mixture was diluted with DCM (20 mL) and poured into H₂O (10 mL). The aqueous layer was extracted with DCM (20 mL), and the combined organic layers were washed with brine (9 mL), dried, filtered and concentrated under reduced pressure. Purification by reverse phase chromatography (50→100% MeCN/H₂O) afforded the desired product as a pale yellow powder (200 mg, 81% yield). LCMS (ESI) m/z: [M+Na] calcd for C₅₉H₈₄N₂O₁₇: 1115.6; found 1115.8.

### Monomer 16. 32(R),40-O,O-bis[(4-nitrophenoxy)carbonyl]rapamycin

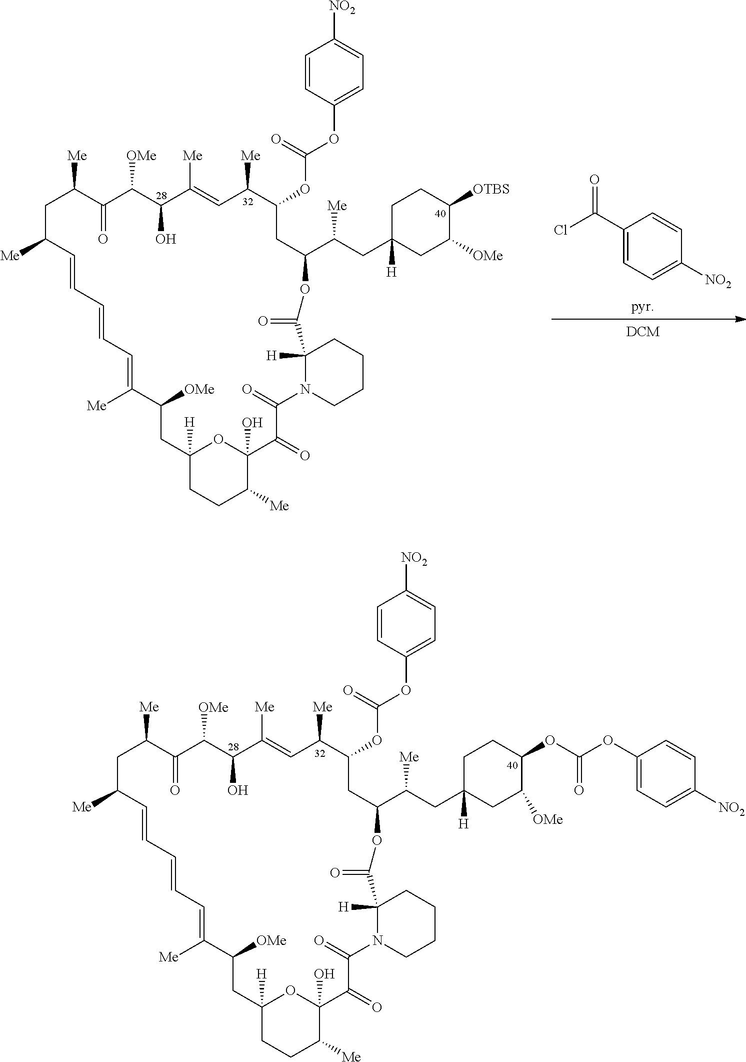

To a solution of 32(R)—O-[(4-nitrophenoxy)carbonyl]rapamycin (675 mg, 0.624 mmol, 1.0 equiv) in DCM (13 mL) was added powdered 4 Å molecular sieves (675 mg). The suspension was stirred for 1 h, at which point pyridine (0.56 mL, 6.90 mmol, 11.1 equiv) was added. The mixture was cooled to −15° C. and then p-nitrophenyl chloroformate (132 mg, 0.655 mmol, 1.05 equiv) was added in one portion. The mixture was warmed to 0° C., stirred for 4 h, and then warmed to room temperature. The reaction mixture was filtered and washed with DCM (25 mL). The filtrate was washed with sat. aqueous NaHCO₃ (15 mL), H₂O (15 mL), and brine (10 mL), dried, filtered, and concentrated under reduced pressure. Purification by silica gel chromatography (25→45% EtOAc/hexanes) afforded the desired product as a faint yellow solid (566 mg, 73% yield). LC-MS (ESI) m/z: [M+Na] calcd for C₆₅H₈₇N₃O₂₁. 1268.57; found 1269.3.

### Monomer 17. 28(R),32(R),40(R)—O,O,O-tris[(4-nitrophenoxy)carbonyl]rapamycin

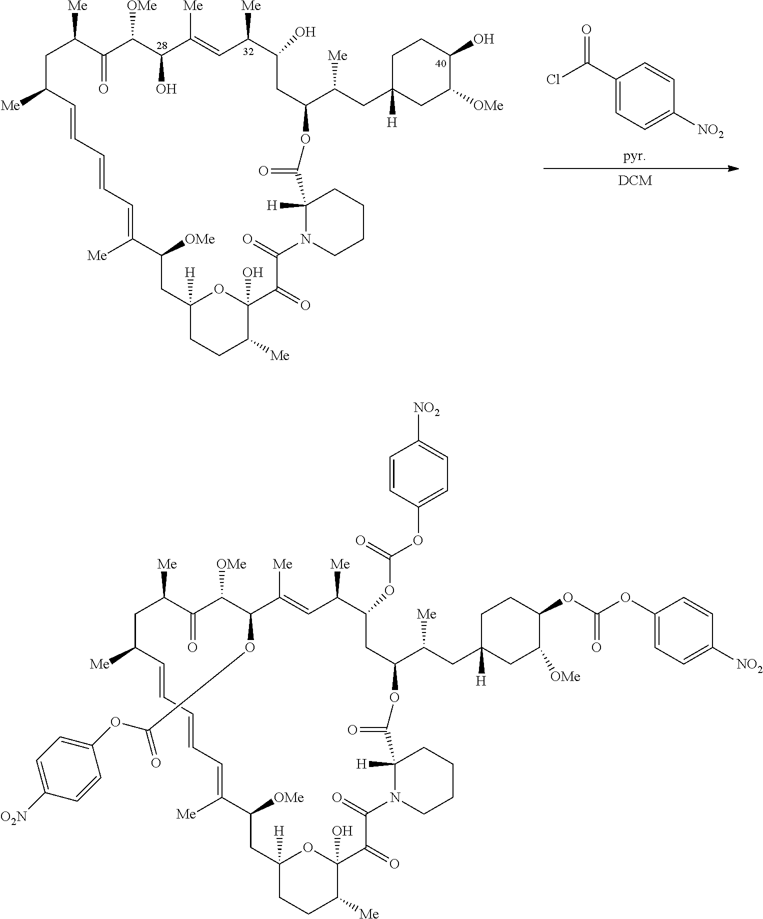

To a solution of 32(R)-hydroxy rapamycin (1.00 g, 1.09 mmol, 1.0 equiv) in DCM (22 mL) was added powdered 4 Å molecular sieves (1.0 g). The suspension was stirred for 45 min, at which point pyridine (0.97 mL, 12.0 mmol, 11.0 equiv) was added. The mixture was cooled to −15° C. and then p-nitrophenyl chloroformate (550 mg, 2.73 mmol, 2.5 equiv) was added in one portion. The mixture was warmed to room temperature over 4 h and stirred overnight. The mixture was cooled to 0° C. and additional p-nitrophenyl chloroformate (220 mg, 1.09 mmol, 1.0 equiv) was added in one portion. The reaction mixture was stirred for 1 h, warmed to room temperature, and then stirred for 2 h. The mixture was once again cooled to 0° C. and additional p-nitrophenyl chloroformate (660 mg, 3.27 mmol, 3.0 equiv) was added. The reaction mixture was stirred for 15 min and then at room temperature for 1 h. The reaction mixture was filtered and washed with DCM (25 mL). The filtrate was washed with sat. aqueous NaHCO₃ (20 mL), H₂O (20 mL), and brine (15 mL), dried, filtered, and concentrated under reduced pressure. Purification by silica gel chromatography (5→15% EtOAc/DCM) afforded the desired product as a faint yellow solid (550 mg, 36% yield). LC-MS (ESI) m/z: [M+Na] calcd for C₇₂H₉₀N₄O₂₅: 1433.58; found 1434.3.

### General Procedures and Specific Examples

### General Procedure 1: Coupling of a Carboxylic Acid and an Amine Followed by N-Boc Deprotection

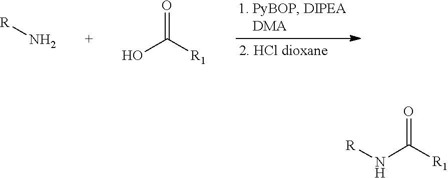

### Step 1

To a 0.1 M solution of carboxylic acid (1.0 equiv) in DMA was added an amine (1.2 equiv), DIPEA (4.0 equiv) and PyBOP (1.3 equiv). The reaction was allowed to stir until consumption of the carboxylic acid, as indicated by LCMS. The reaction mixture was then purified by silica gel chromatography to afford the product.

### Step 2

To a 0.07 M solution of N-Boc protected amine (1.0 equiv) in dioxane was added HCl (4 M in dioxane) (50 equiv). The reaction was allowed to stir until consumption of N-Boc protected amine, as indicated by LCMS. Then the reaction was concentrated to an oil, which was then dissolved in H₂O and lyophilized to afford the product.

### Intermediate A1-7. 1-amino-27-(6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-yl)-3,6,9,12,15,18,21,24-octaoxaheptacosan-27-one

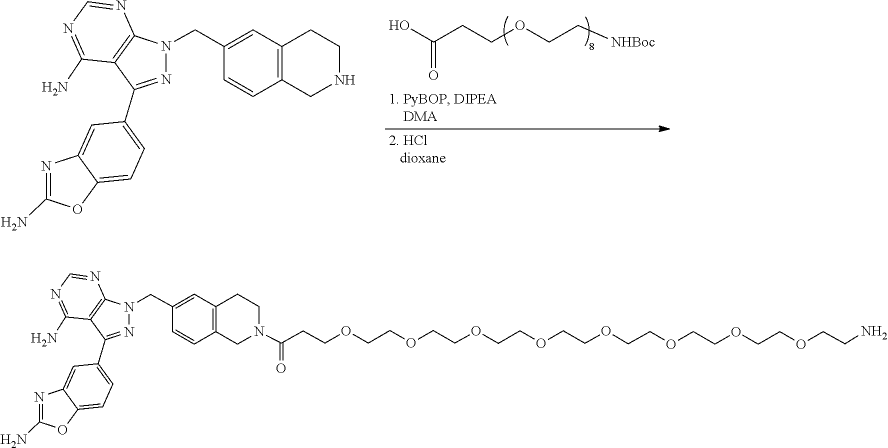

### Step 1: Synthesis of tert-butyl N-[27-(6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-yl)-27-oxo-3,6,9,12,15,18,21,24-octaoxaheptacosan-1-yl]carbamatecarbamate

To a solution of 1-{[(tert-butoxy)carbonyl]amino}-3,6,9,12,15,18,21,24-octaoxaheptacosan-27-oic acid (102 mg, 189 μmol, 1.0 equiv) and 6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-ium (120 mg, 227 μmol, 1.2 equiv) in DMA (1.88 mL) was added DIPEA (131 μL, 756 μmol, 4.0 equiv) followed by PyBOP (127 mg, 245 μmol, 1.3 equiv). The reaction was stirred at room temperature. After 2 h, the reaction mixture was concentrated under reduced pressure and the crude residue was purified by silica gel chromatography (0→20% MeOH/DCM) to give the product (161.5 mg, 91% yield) as a pale yellow oil. LCMS (ESI) m/z: [M+H] calcd for C₄₆H₆₅N₉O₁₂: 936.49; found 936.3.

### Step 2: Synthesis of 1-amino-27-(6-{[4-amino-3-(2-amino-1,3-benzoxazol-5-yl)-1H-pyrazolo[3,4-d]pyrimidin-1-yl]methyl}-1,2,3,4-tetrahydroisoquinolin-2-yl)-3,6,9,12,15,18,21,24-octaoxaheptacosan-27-one

To a solution of tert-butyl N-[27-(6-{[4-amino-3-(2-amino-1,3-benzoxazol-5-yl)-1H-pyrazolo[3,4-d]pyrimidin-1l-yl]methyl}-1,2,3,4-tetrahydroisoquinolin-2-yl)-27-oxo-3,6,9,12,15,18,21,24-octaoxaheptacosan-1-yl]carbamate (0.9 g, 0.9614 mmol, 1.0 equiv) in dioxane (3.20 mL) was added HCl (4 M in dioxane, 2.40 mL, 9.61 mmol, 10.0 equiv). The reaction stirred for 2 h and then was concentrated under reduced pressure to an oil. The oil was azeotroped with DCM (3×15 mL) to provide the product (881 mg, 10500 yield, HCl) as a tan solid, which was used directly in the next step. LCMS (ESI) m/z: [M+H] calcd for C₄₁NO₅₇N₉O₁₀: 836.43; found 836.3.

Following General Procedure 1, but using the appropriate amine-containing active site inhibitor in Table 2 and PEG carboxylic acid, the Intermediates A1 in Table 5 were prepared:

TABLE 5
Additional amines prepared

| Structure | Molecular Formula | Calculated MW | Observed MW |
|---|---|---|---|
| 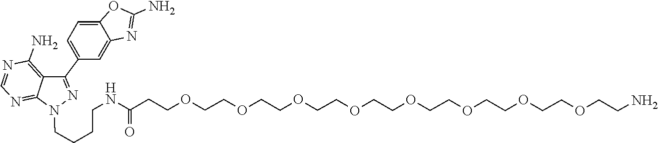 [SMILES: NCCOCCOCCOCCOCCOCCOCCOCCOCCC(=O)NCCCCn1nc(-c2ccc3oc(N)nc3c2)c2c(N)ncnc21] | C₃₅H₅₅N₉O₁₀ | [M + H] = 762.42 | [M + H] = 762.3 |
| Intermediate A1-1 | | | |
| 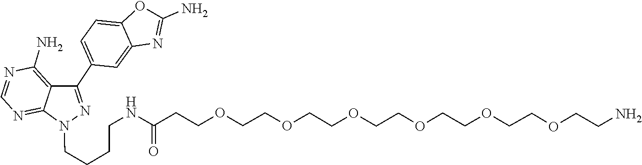 [SMILES: NCCOCCOCCOCCOCCOCCOCCC(=O)NCCCCn1nc(-c2ccc3oc(N)nc3c2)c2c(N)ncnc21] | C₃₁H₄₇N₉O₈ | [M + H] = 674.36 | [M + H] = 674.3 |
| Intermediate A1-2 | | | |
| 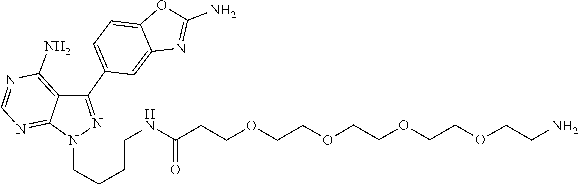 [SMILES: NCCOCCOCCOCCOCCC(=O)NCCCCn1nc(-c2ccc3oc(N)nc3c2)c2c(N)ncnc21] | C₂₇H₃₉N₉O₆ | [M + H] = 586.31 | [M + H] = 586.6 |
| Intermediate A1-3 | | | |
| 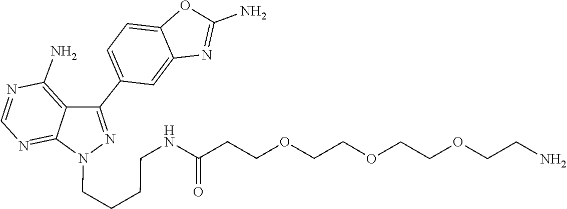 [SMILES: NCCOCCOCCOCCC(=O)NCCCCn1nc(-c2ccc3oc(N)nc3c2)c2c(N)ncnc21] | C₂₅H₃₅N₉O₅ | [M + H] = 542.29 | [M + H] = 542.3 |
| Intermediate A1-4 | | | |
| 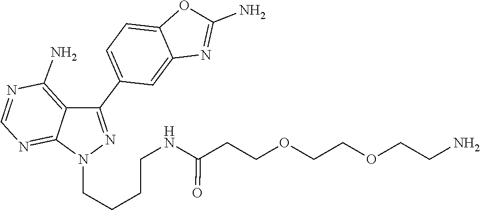 [SMILES: NCCOCCOCCC(=O)NCCCCn1nc(-c2ccc3oc(N)nc3c2)c2c(N)ncnc21] | C₂₃H₃₁N₉O₄ | [M + H] = 498.26 | [M + H] = 498.2 |
| Intermediate A1-5 | | | |
| 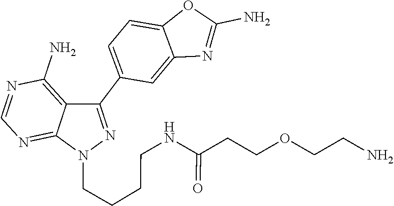 [SMILES: NCCOCCC(=O)NCCCCn1nc(-c2ccc3oc(N)nc3c2)c2c(N)ncnc21] | C₂₁H₂₇N₉O₃ | [M + H] = 454.23 | [M + H] = 454.1 |
| Intermediate A1-6 | | | |
| 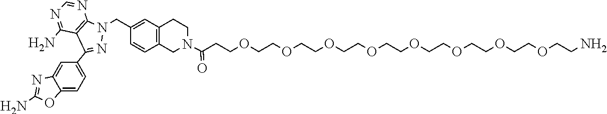 [SMILES: NCCOCCOCCOCCOCCOCCOCCOCCOCCC(=O)N1CCc2cc(Cn3nc(-c4ccc5oc(N)nc5c4)c4c(N)ncnc43)ccc2C1] | C₄₁H₅₇N₉O₁₀ | [M + H] = 836.43 | [M + H] = 836.3 |
| Intermediate A1-7 | | | |
| 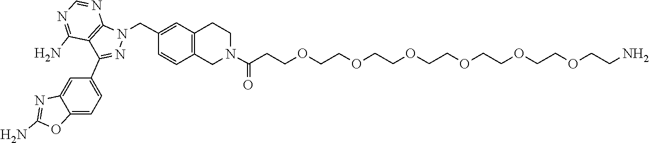 [SMILES: NCCOCCOCCOCCOCCOCCOCCC(=O)N1CCc2cc(Cn3nc(-c4ccc5oc(N)nc5c4)c4c(N)ncnc43)ccc2C1] | C₃₇H₄₉N₉O₈ | [M + H] = 748.38 | [M + H] = 748.2 |
| Intermediate A1-8 | | | |
| 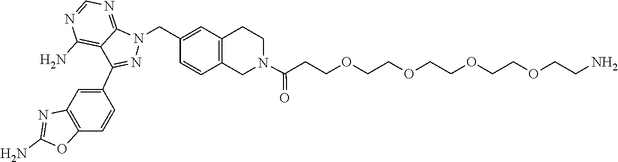 [SMILES: NCCOCCOCCOCCOCCC(=O)N1CCc2cc(Cn3nc(-c4ccc5oc(N)nc5c4)c4c(N)ncnc43)ccc2C1] | C₃₃H₄₁N₉O₆ | [M + H] = 660.33 | [M + H] = 660.2 |
| Intermediate A1-9 | | | |
| 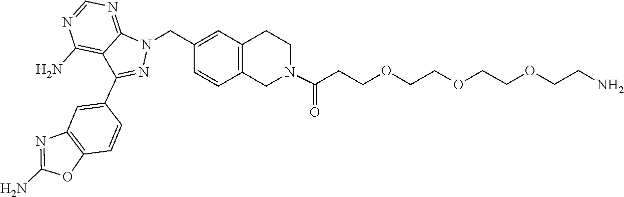 [SMILES: NCCOCCOCCOCCC(=O)N1CCc2cc(Cn3nc(-c4ccc5oc(N)nc5c4)c4c(N)ncnc43)ccc2C1] | C₃₁H₃₇N₉O₅ | [M + H] = 616.30 | [M + H] = 616.3 |
| Intermediate A1-10 | | | |
| 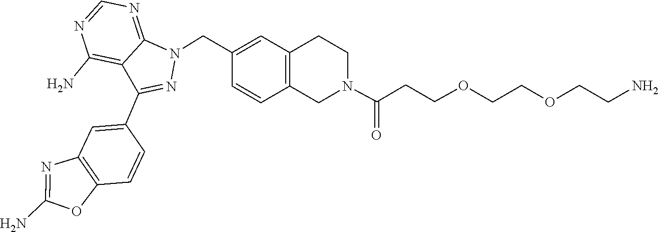 [SMILES: NCCOCCOCCC(=O)N1CCc2cc(Cn3nc(-c4ccc5oc(N)nc5c4)c4c(N)ncnc43)ccc2C1] | C₂₉H₃₃N₉O₄ | [M + H] = 572.28 | [M + H] = 572.3 |
| Intermediate A1-11 | | | |
| 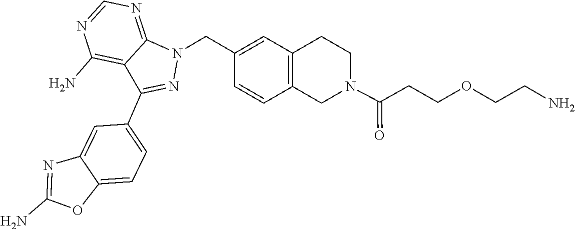 [SMILES: NCCOCCC(=O)N1CCc2cc(Cn3nc(-c4ccc5oc(N)nc5c4)c4c(N)ncnc43)ccc2C1] | C₂₇H₂₉N₉O₃ | [M + H] = 528.25 | [M + H] = 528.2 |
| Intermediate A1-12 | | | |
| 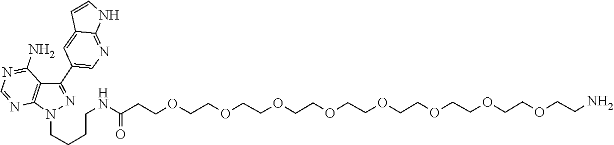 [SMILES: NCCOCCOCCOCCOCCOCCOCCOCCOCCC(=O)NCCCCn1nc(-c2cnc3[nH]ccc3c2)c2c(N)ncnc21] | C₃₅H₅₅N₉O₉ | [M + H] = 746.42 | [M + H] = 746.4 |
| Intermediate A1-13 | | | |
| 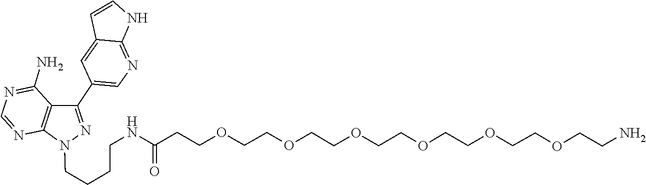 [SMILES: NCCOCCOCCOCCOCCOCCOCCC(=O)NCCCCn1nc(-c2cnc3[nH]ccc3c2)c2c(N)ncnc21] | C₃₁H₄₇N₉O₇ | [M + H] = 658.37 | [M + H] = 658.3 |
| Intermediate A1-14 | | | |
| 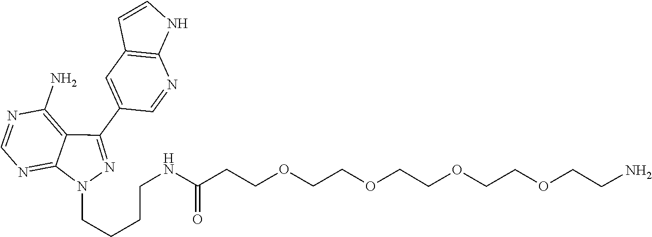 [SMILES: NCCOCCOCCOCCOCCC(=O)NCCCCn1nc(-c2cnc3[nH]ccc3c2)c2c(N)ncnc21] | C₂₇H₃₉N₉O₅ | [M + H] = 570.32 | [M + H] = 570.2 |
| Intermediate A1-15 | | | |
| 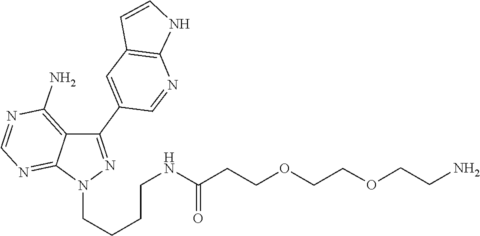 [SMILES: NCCOCCOCCC(=O)NCCCCn1nc(-c2cnc3[nH]ccc3c2)c2c(N)ncnc21] | C₂₃H₃₁N₉O₃ | [M + H] = 482.26 | [M + H] = 482.3 |
| Intermediate A1-16 | | | |
| 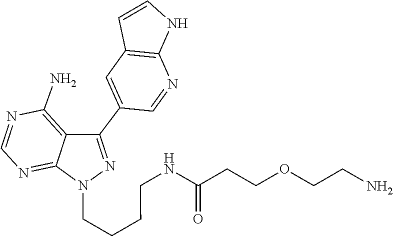 [SMILES: NCCOCCC(=O)NCCCCn1nc(-c2cnc3[nH]ccc3c2)c2c(N)ncnc21] | C₂₁H₂₇N₉O₂ | [M + H] = 438.24 | [M + H] = 438.4 |
| Intermediate A1-17 | | | |
| 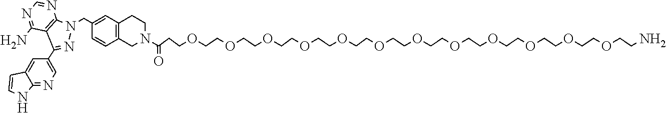 [SMILES: NCCOCCOCCOCCOCCOCCOCCOCCOCCOCCOCCOCCOCCC(=O)N1CCc2cc(Cn3nc(-c4cnc5[nH]ccc5c4)c4c(N)ncnc43)ccc2C1] | C₄₉H₇₃N₉O₁₃ | [M + H] = 996.54 | [M + H] = 996.4 |
| Intermediate A1-18 | | | |
| 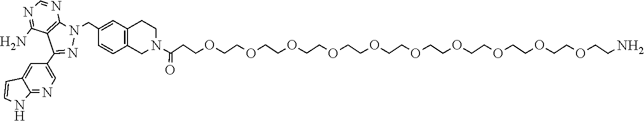 [SMILES: NCCOCCOCCOCCOCCOCCOCCOCCOCCOCCOCCC(=O)N1CCc2cc(Cn3nc(-c4cnc5[nH]ccc5c4)c4c(N)ncnc43)ccc2C1] | C₄₅H₆₅N₉O₁₁ | [M + H] = 908.49 | [M + H] = 908.3 |
| Intermediate A1-19 | | | |
| 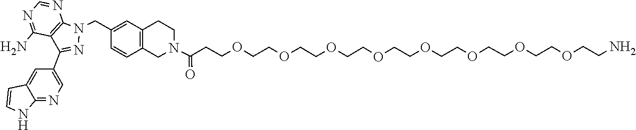 [SMILES: NCCOCCOCCOCCOCCOCCOCCOCCOCCC(=O)N1CCc2cc(Cn3nc(-c4cnc5[nH]ccc5c4)c4c(N)ncnc43)ccc2C1] | C₄₁H₅₇N₉O₉ | [M + H] = 820.44 | [M + H] = 820.3 |
| Intermediate A1-20 | | | |
| 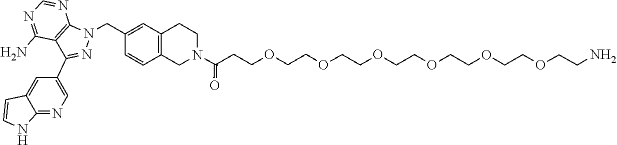 [SMILES: NCCOCCOCCOCCOCCOCCOCCC(=O)N1CCc2cc(Cn3nc(-c4cnc5[nH]ccc5c4)c4c(N)ncnc43)ccc2C1] | C₃₇H₄₉N₉O₇ | [M + H] = 732.39 | [M + H] = 732.3 |
| Intermediate A1-21 | | | |
| 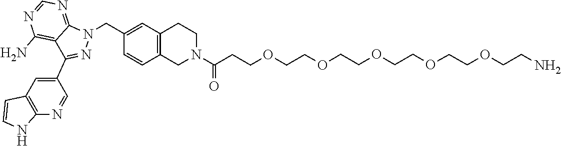 [SMILES: NCCOCCOCCOCCOCCOCCC(=O)N1CCc2cc(Cn3nc(-c4cnc5[nH]ccc5c4)c4c(N)ncnc43)ccc2C1] | C₃₅H₄₅N₉O₆ | [M + H] = 688.36 | [M + H] = 688.3 |
| Intermediate A1-22 | | | |
| 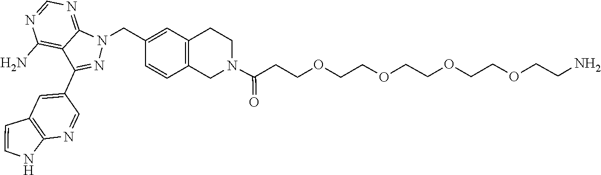 [SMILES: NCCOCCOCCOCCOCCC(=O)N1CCc2cc(Cn3nc(-c4cnc5[nH]ccc5c4)c4c(N)ncnc43)ccc2C1] | C₃₃H₄₁N₉O₅ | [M + H] = 644.33 | [M + H] = 644.3 |
| Intermediate A1-23 | | | |
| 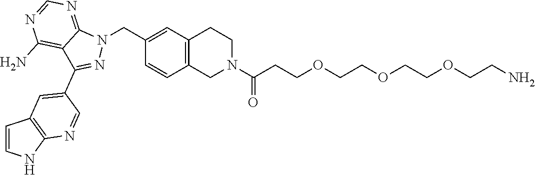 [SMILES: NCCOCCOCCOCCC(=O)N1CCc2cc(Cn3nc(-c4cnc5[nH]ccc5c4)c4c(N)ncnc43)ccc2C1] | C₃₁H₃₇N₉O₄ | [M + H] = 600.31 | [M + H] = 600.4 |
| Intermediate A1-24 | | | |
| 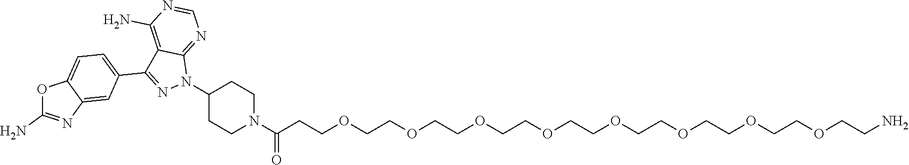 [SMILES: NCCOCCOCCOCCOCCOCCOCCOCCOCCC(=O)N1CCC(n2nc(-c3ccc4oc(N)nc4c3)c3c(N)ncnc32)CC1] | C₃₆H₅₅N₉O₁₀ | [M + H] = 774.42 | [M + H] = 774.7 |
| Intermediate A1-25 | | | |
| 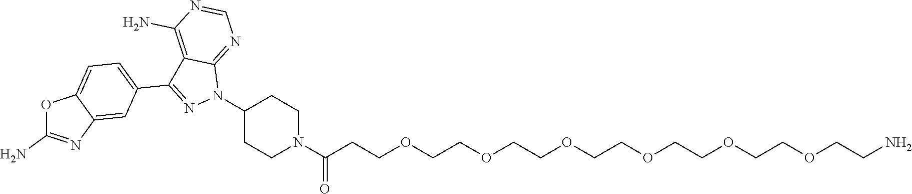 [SMILES: NCCOCCOCCOCCOCCOCCOCCC(=O)N1CCC(n2nc(-c3ccc4oc(N)nc4c3)c3c(N)ncnc32)CC1] | C₃₂H₄₇N₉O₈ | [M + H] = 686.36 | [M + H] = 686.4 |
| Intermediate A1-26 | | | |
| 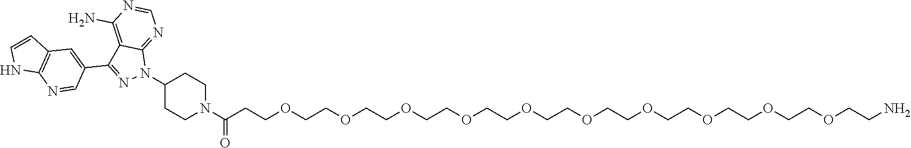 [SMILES: NCCOCCOCCOCCOCCOCCOCCOCCOCCOCCOCCC(=O)N1CCC(n2nc(-c3cnc4[nH]ccc4c3)c3c(N)ncnc32)CC1] | C₄₀H₆₃N₉O₁₁ | [M + H] = 846.47 | [M + H] = 846.8 |
| Intermediate A1-27 | | | |
| 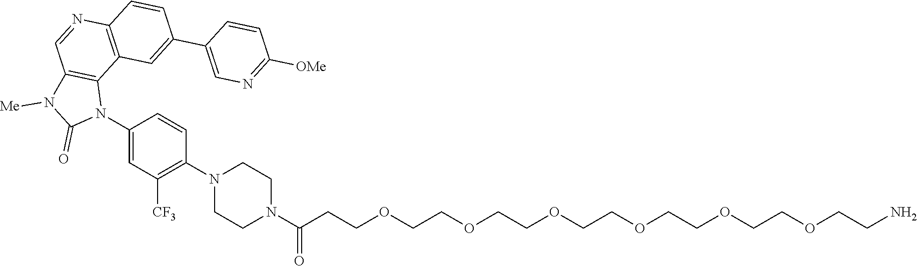 [SMILES: COc1ccc(-c2ccc3ncc4c(c3c2)n(-c2ccc(N3CCN(C(=O)CCOCCOCCOCCOCCOCCOCCN)CC3)c(C(F)(F)F)c2)c(=O)n4C)cn1] | C₄₃H₅₄F₃N₇O₉ | [M + H] = 870.40 | [M + H] = 870.4 |
| Intermediate A1-28 | | | |
| 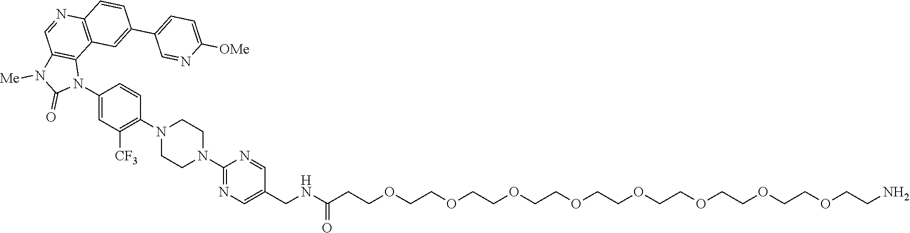 [SMILES: COc1ccc(-c2ccc3ncc4c(c3c2)n(-c2ccc(N3CCN(c5ncc(CNC(=O)CCOCCOCCOCCOCCOCCOCCOCCOCCN)cn5)CC3)c(C(F)(F)F)c2)c(=O)n4C)cn1] | C₅₂H₆₇F₃N₁₀O₁₁ | [M + H] = 1065.50 | [M + H] = 1065.4 |
| Intermediate A1-29 | | | |
| 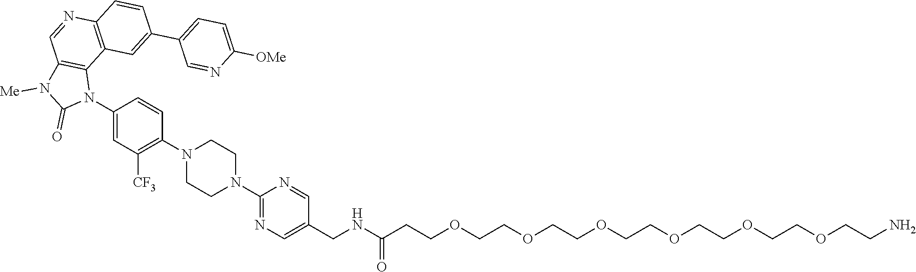 [SMILES: COc1ccc(-c2ccc3ncc4c(c3c2)n(-c2ccc(N3CCN(c5ncc(CNC(=O)CCOCCOCCOCCOCCOCCOCCN)cn5)CC3)c(C(F)(F)F)c2)c(=O)n4C)cn1] | C₄₈H₅₉F₃N₁₀O₉ | [M + H] = 977.45 | [M + H] = 977.4 |
| Intermediate A1-30 | | | |
| 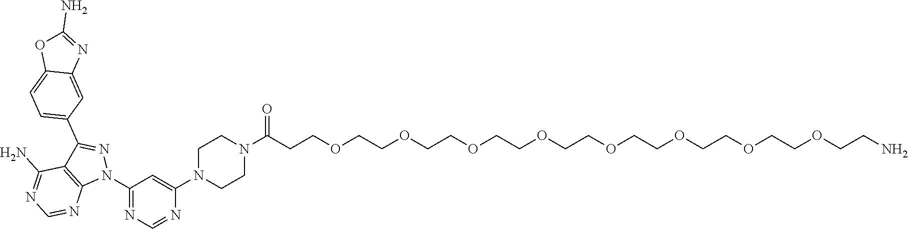 [SMILES: NCCOCCOCCOCCOCCOCCOCCOCCOCCC(=O)N1CCN(c2cc(-n3nc(-c4ccc5oc(N)nc5c4)c4c(N)ncnc43)ncn2)CC1] | C₃₉H₅₇F₃N₁₂O₁₂ | [M + H] = 853.43 | [M + H] = 853.4 |
| Intermediate A1-31 | | | |
| 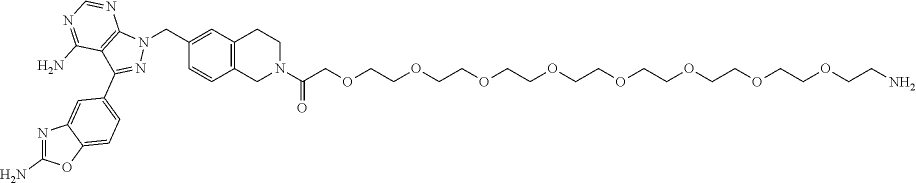 [SMILES: NCCOCCOCCOCCOCCOCCOCCOCCOCC(=O)N1CCc2cc(Cn3nc(-c4ccc5oc(N)nc5c4)c4c(N)ncnc43)ccc2C1] | C₄₀H₅₅N₉O₁₀ | [M + H] = 822.42 | [M + H] = 822.2 |
| Intermediate A1-32 | | | |
| 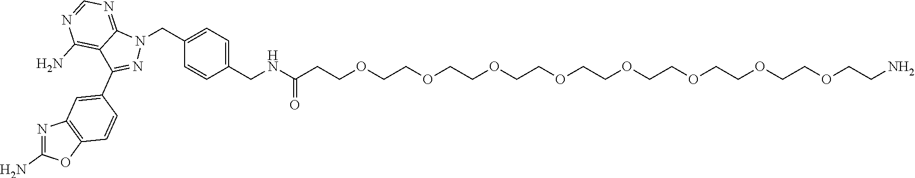 [SMILES: NCCOCCOCCOCCOCCOCCOCCOCCOCCC(=O)NCc1ccc(Cn2nc(-c3ccc4oc(N)nc4c3)c3c(N)ncnc32)cc1] | C₃₉H₅₅N₉O₁₀ | [M + H] = 810.42 | [M + H] = 810.3 |
| Intermediate A1-33 | | | |
| 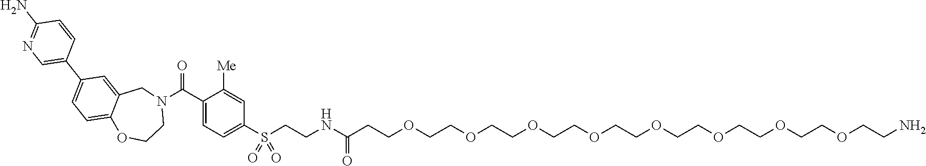 [SMILES: Cc1cc(S(=O)(=O)CCNC(=O)CCOCCOCCOCCOCCOCCOCCOCCOCCN)ccc1C(=O)N1CCOc2ccc(-c3ccc(N)nc3)cc2C1] | C₄₃H₆₃N₅O₁₃S | [M + H] = 890.42 | [M + H] = 890.3 |
| Intermediate A1-34 | | | |
| 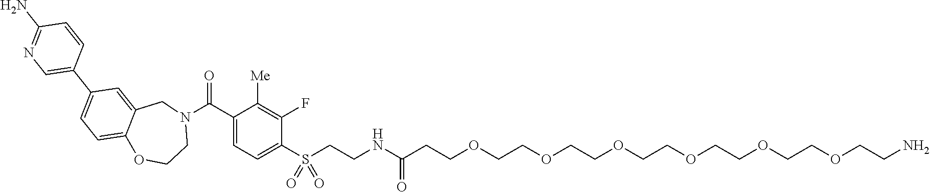 [SMILES: Cc1c(C(=O)N2CCOc3ccc(-c4ccc(N)nc4)cc3C2)ccc(S(=O)(=O)CCNC(=O)CCOCCOCCOCCOCCOCCOCCN)c1F] | C₃₉H₅₄FN₅O₁₁S | [M + H] = 820.36 | [M + H] = 820.3 |
| Intermediate A1-35 | | | |
| 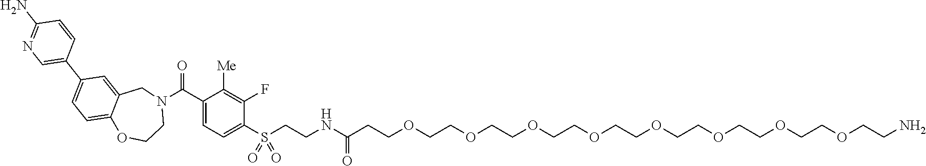 [SMILES: Cc1c(C(=O)N2CCOc3ccc(-c4ccc(N)nc4)cc3C2)ccc(S(=O)(=O)CCNC(=O)CCOCCOCCOCCOCCOCCOCCOCCOCCN)c1F] | C₄₃H₆₂FN₅O₁₃S | [M + H] = 908.41 | [M + H] = 908.3 |
| Intermediate A1-36 | | | |
| 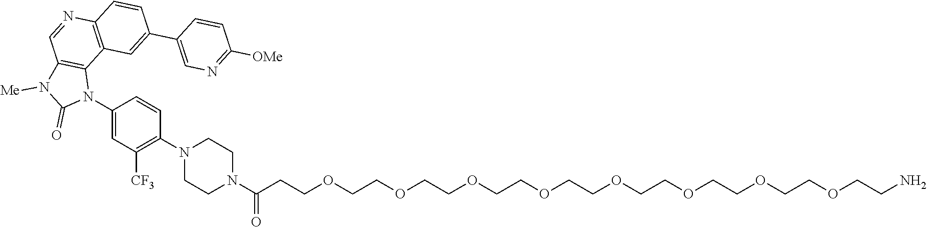 [SMILES: COc1ccc(-c2ccc3ncc4c(c3c2)n(-c2ccc(N3CCN(C(=O)CCOCCOCCOCCOCCOCCOCCOCCOCCN)CC3)c(C(F)(F)F)c2)c(=O)n4C)cn1] | C₄₇H₆₂F₃N₇O₁₁ | [M + H] = 958.46 | [M + H] = 958.3 |
| Intermediate A1-37 | | | |
| 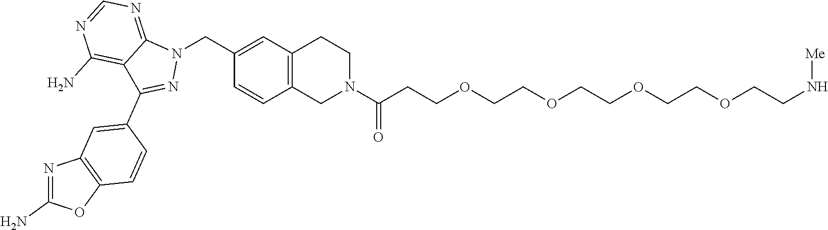 [SMILES: CNCCOCCOCCOCCOCCC(=O)N1CCc2cc(Cn3nc(-c4ccc5oc(N)nc5c4)c4c(N)ncnc43)ccc2C1] | C₃₄H₄₃N₉O₆ | [M + H] = 674.34 | [M + H] = 674.3 |
| Intermediate A1-38 | | | |
| 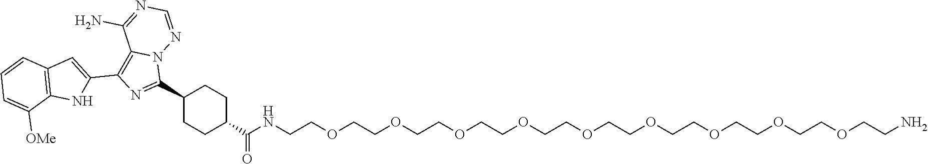 [SMILES: COc1cccc2cc(-c3nc([C@H]4CC[C@H](C(=O)NCCOCCOCCOCCOCCOCCOCCOCCOCCOCCN)CC4)n4ncnc(N)c34)[nH]c12] | C₄₁H₆₄N₈O₁₁ | [M + H] = 845.48 | [M + H] = 845.3 |
| Intermediate A1-39 | | | |
| 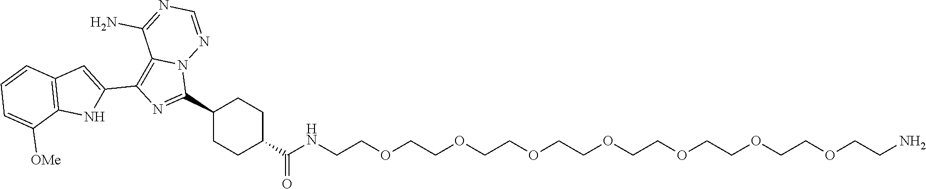 [SMILES: COc1cccc2cc(-c3nc([C@H]4CC[C@H](C(=O)NCCOCCOCCOCCOCCOCCOCCOCCN)CC4)n4ncnc(N)c34)[nH]c12] | C₃₇H₅₆N₈O₉ | [M + H] = 757.43 | [M + H] = 757.3 |
| Intermediate A1-40 | | | |
| 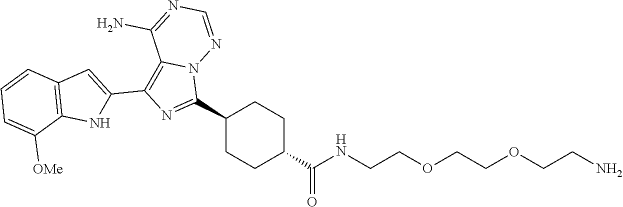 [SMILES: COc1cccc2cc(-c3nc([C@H]4CC[C@H](C(=O)NCCOCCOCCN)CC4)n4ncnc(N)c34)[nH]c12] | C₂₇H₃₆N₈O₄ | [M + H] = 537.29 | [M + H] = 537.2 |
| Intermediate A1-41 | | | |
| 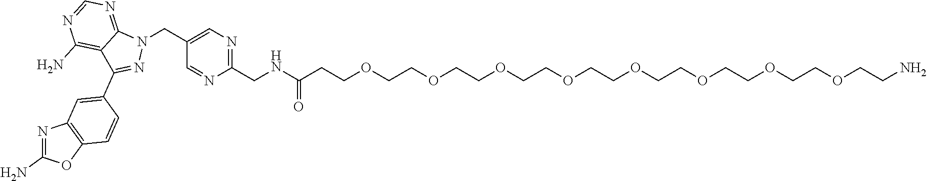 [SMILES: NCCOCCOCCOCCOCCOCCOCCOCCOCCC(=O)NCc1ncc(Cn2nc(-c3ccc4oc(N)nc4c3)c3c(N)ncnc32)cn1] | C₃₇H₅₃N₁₁O₁₀ | [M + H] = 812.41 | [M + H] = 812.3 |
| Intermediate A1-42 | | | |
| 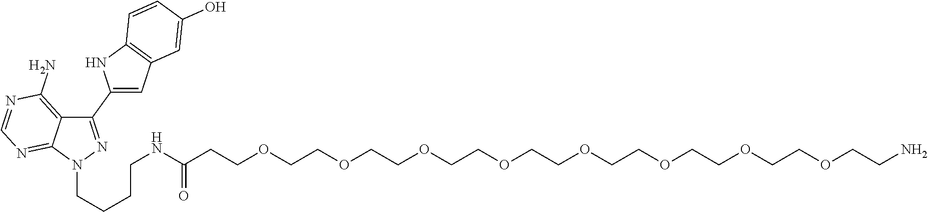 [SMILES: NCCOCCOCCOCCOCCOCCOCCOCCOCCC(=O)NCCCCn1nc(-c2cc3cc(O)ccc3[nH]2)c2c(N)ncnc21] | C₃₆H₅₆N₈O₁₀ | [M + H] = 761.42 | [M + H] = 761.3 |
| Intermediate A1-43 | | | |
| 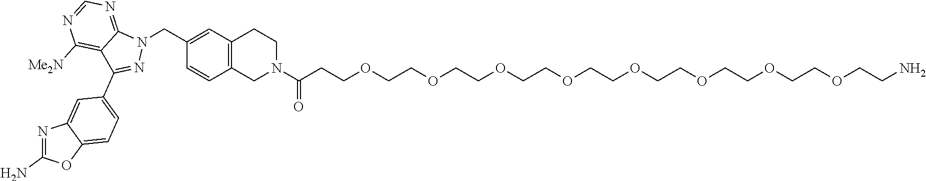 [SMILES: CN(C)c1ncnc2c1c(-c1ccc3oc(N)nc3c1)nn2Cc1ccc2c(c1)CCN(C(=O)CCOCCOCCOCCOCCOCCOCCOCCOCCN)C2] | C₄₃H₆₁N₉O₁₀ | [M + H] = 864.46 | [M + H] = 864.4 |
| Intermediate A1-44 | | | |
| 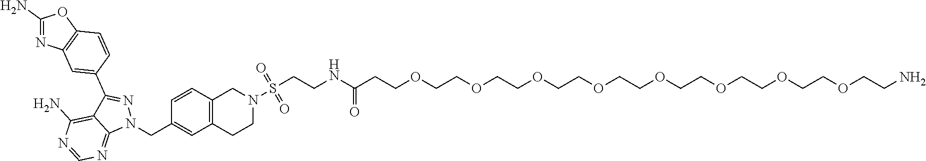 [SMILES: NCCOCCOCCOCCOCCOCCOCCOCCOCCC(=O)NCCS(=O)(=O)N1CCc2cc(Cn3nc(-c4ccc5oc(N)nc5c4)c4c(N)ncnc43)ccc2C1] | C₄₃H₆₂N₁₀O₁₂S | [M + H] = 943.44 | [M + H] = 943.3 |
| Intermediate A1-45 | | | |
| 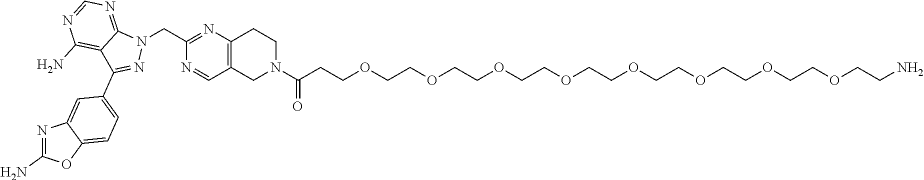 [SMILES: NCCOCCOCCOCCOCCOCCOCCOCCOCCC(=O)N1CCc2nc(Cn3nc(-c4ccc5oc(N)nc5c4)c4c(N)ncnc43)ncc2C1] | C₃₉H₅₅N₁₁O₁₀ | [M + H] = 838.42 | [M + H] = 838.3 |
| Intermediate A1-46 | | | |

Following General Procedure 1, but using the appropriate Intermediate A1 in Table 5 and PEG carboxylic acid, the Intermediates A2 in Table 6 were prepared:

TABLE 6
Additional amines prepared
Structure

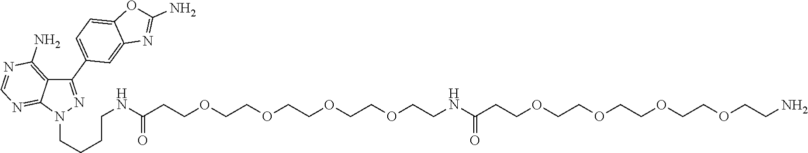 [SMILES: NCCOCCOCCOCCOCCC(=O)NCCOCCOCCOCCOCCC(=O)NCCCCn1nc(-c2ccc3oc(N)nc3c2)c2c(N)ncnc21]

Intermediate A2-1

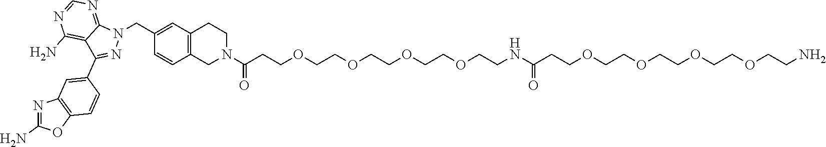 [SMILES: NCCOCCOCCOCCOCCC(=O)NCCOCCOCCOCCOCCC(=O)N1CCc2cc(Cn3nc(-c4ccc5oc(N)nc5c4)c4c(N)ncnc43)ccc2C1]

Intermediate A2-2

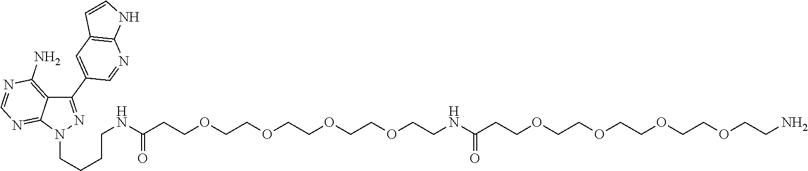 [SMILES: NCCOCCOCCOCCOCCC(=O)NCCOCCOCCOCCOCCC(=O)NCCCCn1nc(-c2cnc3[nH]ccc3c2)c2c(N)ncnc21]

Intermediate A2-3

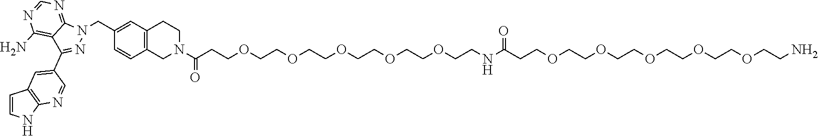 [SMILES: NCCOCCOCCOCCOCCOCCC(=O)NCCOCCOCCOCCOCCOCCC(=O)N1CCc2cc(Cn3nc(-c4cnc5[nH]ccc5c4)c4c(N)ncnc43)ccc2C1]

Intermediate A2-4

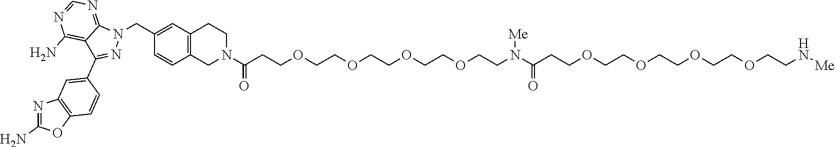 [SMILES: CNCCOCCOCCOCCOCCC(=O)N(C)CCOCCOCCOCCOCCC(=O)N1CCc2cc(Cn3nc(-c4ccc5oc(N)nc5c4)c4c(N)ncnc43)ccc2C1]

Intermediate A2-5

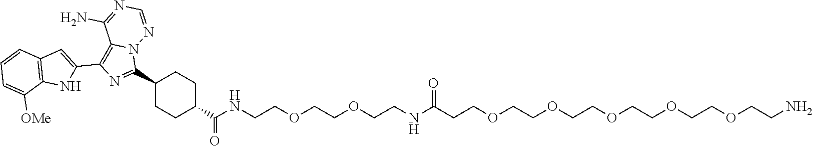 [SMILES: COc1cccc2cc(-c3nc([C@H]4CC[C@H](C(=O)NCCOCCOCCNC(=O)CCOCCOCCOCCOCCOCCN)CC4)n4ncnc(N)c34)[nH]c12]

Intermediate A2-6

| Structure | Molecular Formula | Calculated MW | Observed MW |
|---|---|---|---|
| Intermediate A2-1 | C₃₈H₆₀N₁₀O₁₁ | [M + H] = 833.45 | [M + H] = 833.8 |
| Intermediate A2-2 | C₄₄H₆₂N₁₀O₁₁ | [M + H] = 907.47 | [M + H] = 908.0 |
| Intermediate A2-3 | C₃₈H₆₀N₁₀O₁₀ | [M + H] = 817.46 | [M + H] = 817.4 |
| Intermediate A2-4 | C₄₈H₇₀N₁₀O₁₂ | [M + H] = 979.53 | [M + H] = 979.9 |
| Intermediate A2-5 | C₄₆H₆₆N₁₀O₁₁ | [M + H] = 935.50 | [M + H] = 935.3 |
| Intermediate A2-6 | C₄₀H₆₁N₉O₁₀ | [M + H] = 828.46 | [M + H] = 828.3 |

### General Procedure 2: Coupling of a 4-Nitrophenyl Carbonate Containing Rapamycin Monomer and an Active Site Inhibitor Containing Intermediate Having a Primary or Secondary Amine

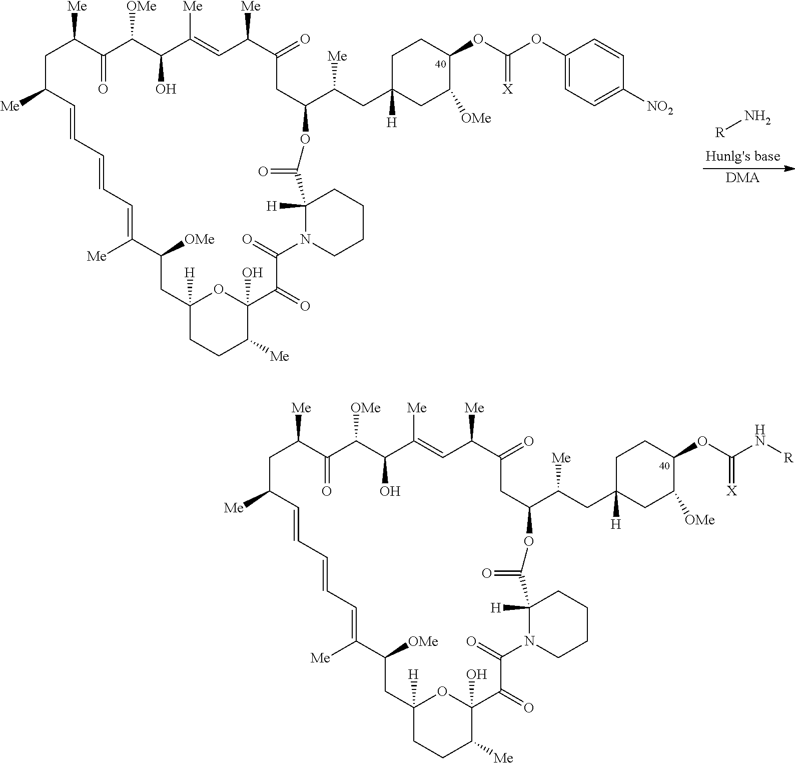

To a 0.02 M solution of 4-nitrophenyl carbonate containing rapamycin monomer (1.0 equiv) and an active site inhibitor containing intermediate (2.0 equiv) in DMA was added DIPEA (4.0 equiv). The resulting solution was stirred at room temperature under nitrogen. Upon completion as determined by LCMS analysis, the crude reaction mixture was purified by preparative HPLC to provide the product.

### Example 2: Synthesis of Series 1 Bivalent Rapamycin Compound

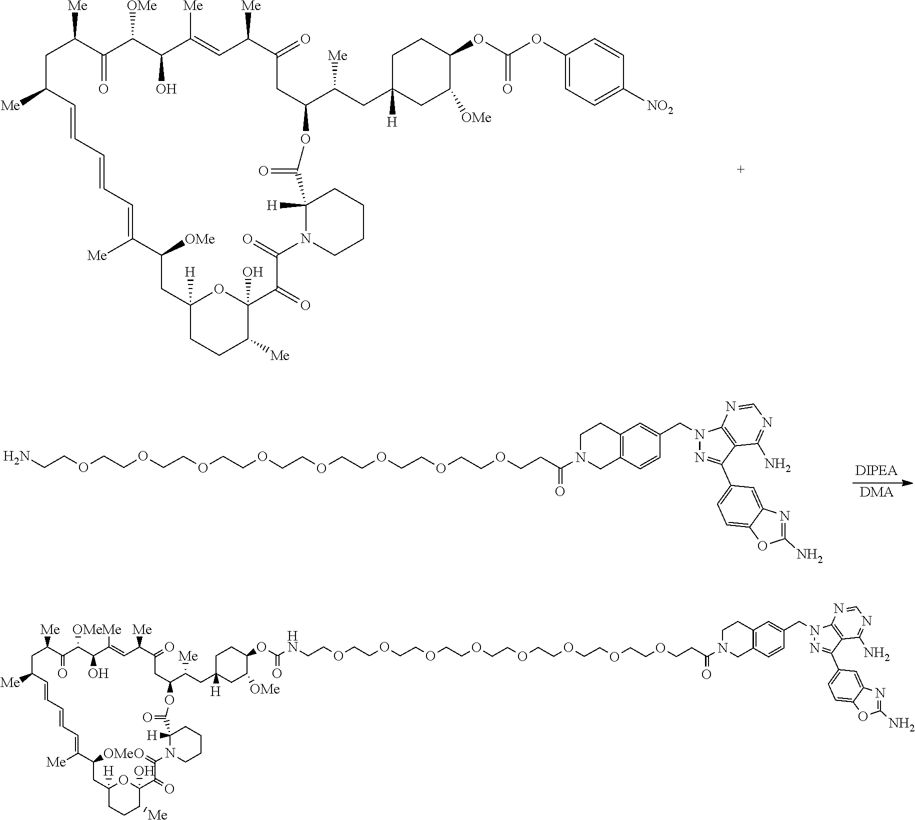

To a solution of 40(R)—O-(4-nitrophenyl carbonate) rapamycin (25 mg, 23.16 μmol, 1.0 equiv) and Intermediate A1-7 (42.0 mg, 46.32 μmol, 2.0 equiv) in DMA (1.15 mL) was added DIPEA (16.0 μL, 92.64 Mmol, 4.0 equiv). The reaction was stirred for 18 h, at which point the reaction mixture purified by reverse phase chromatography (10→40→95% MeCN+0.1% formic acid/1H₂O+0.1% formic acid) to give the product (9.92 mg, 2400 yield) as a white solid. LCMS (ESI) m/z: [M+H] calcd for C₉₃H₁₃₄N₁₀O₂₄: 1775.97; found 1775.7.

Following General Procedure 2, but using the appropriate 4-nitrophenyl carbonate containing rapamycin monomer in Table 1 and Intermediates A1 and A2 from Tables 5 and 6, the Series 1 bivalent analogs in Table 7 were synthesized:

TABLE 7
Series 1 Bivalent Compounds:

| | Molecular | Calculated | Observed |
|---|---|---|---|
| Structure | Formula | MW | MW |
| 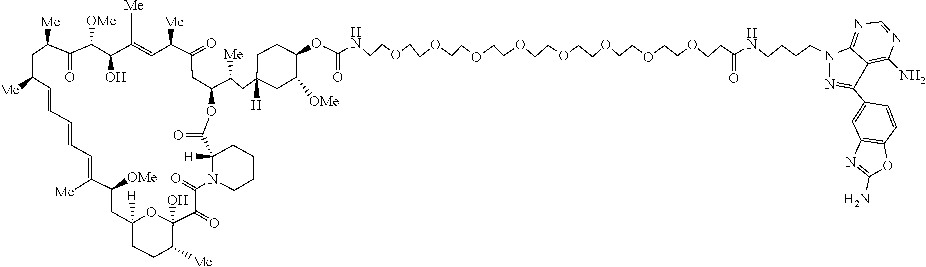 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4ccc5oc(N)nc5c4)c4c(N)ncnc43)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₇H₁₃₂N₁₀O₂₄ | [M + H] = 1701.95 | [M + H] = 1701.8 |
| Example 1 | | | |
| 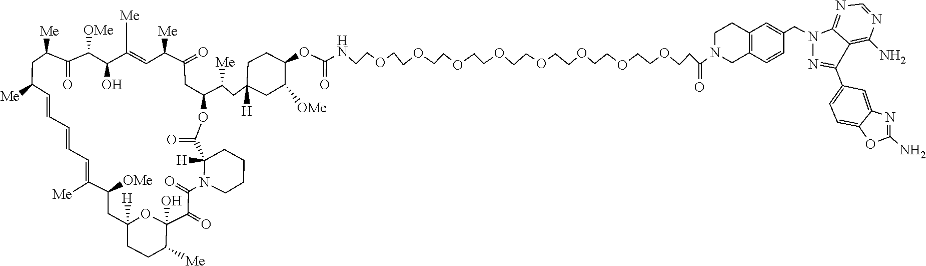 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₃₄N₁₀O₂₄ | [M + H] = 1775.97 | [M + H] = 1775.7 |
| Example 2 | | | |
| 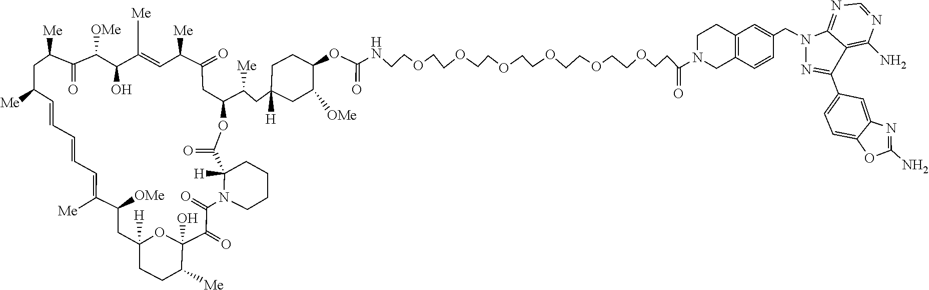 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₉H₁₂₆N₁₀O₂₂ | [M + H] = 1687.91 | [M + H] = 1687.8 |
| Example 3 | | | |
| 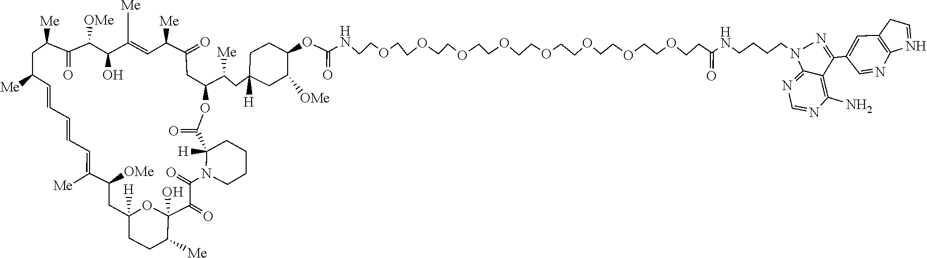 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4cnc5c(c4)CC=N5)c4c(N)ncnc43)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₇H₁₃₂N₁₀O₂₃ | [M + H] = 1684.95 | [M + H] = 1685.1 |
| Example 4 | | | |
| 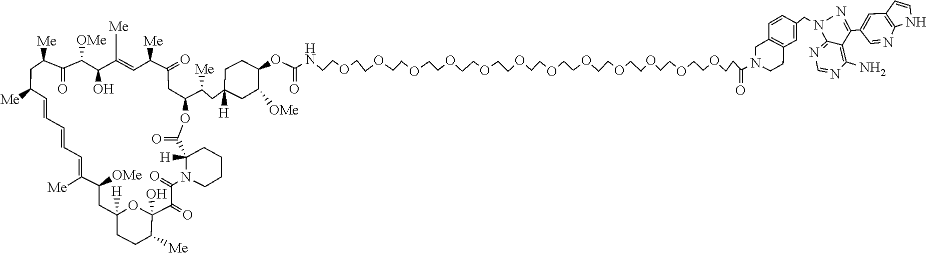 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₁H₁₅₀N₁₀O₂₇ | [M + H] = 1936.08 | [M + H] = 1936.4 |
| Example 5 | | | |
| 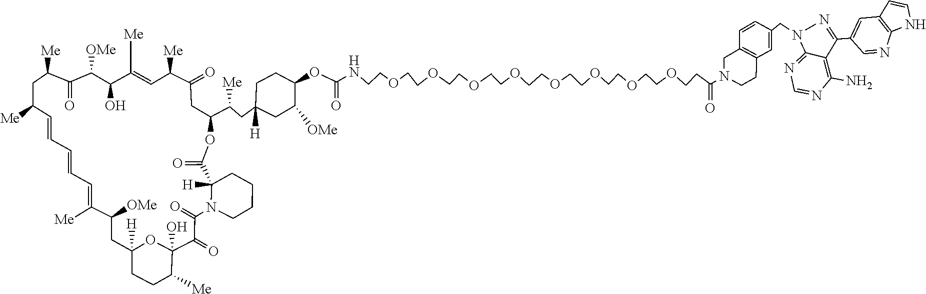 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₄₂N₁₀O₂₅ | [M + H] = 1848.02 | [M + H] = 1848.3 |
| Example 6 | | | |
| 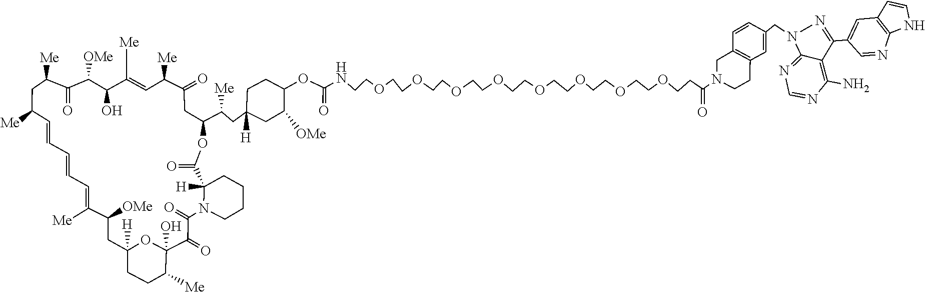 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CCC(OC(=O)NCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₃₄N₁₀O₂₃ | [M + H] = 1759.97 | [M + H] = 1760.1 |
| Example 7 | | | |
| 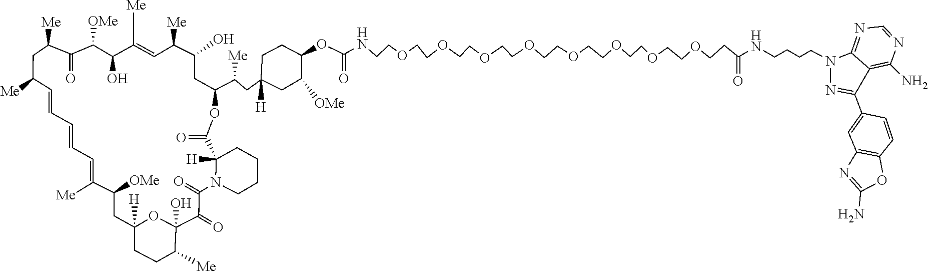 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4ccc5oc(N)nc5c4)c4c(N)ncnc43)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₈₇H₁₃₄N₁₀O₂₄ | [M + H] = 1703.97 | [M + H] = 1703.7 |
| Example 8 | | | |
| 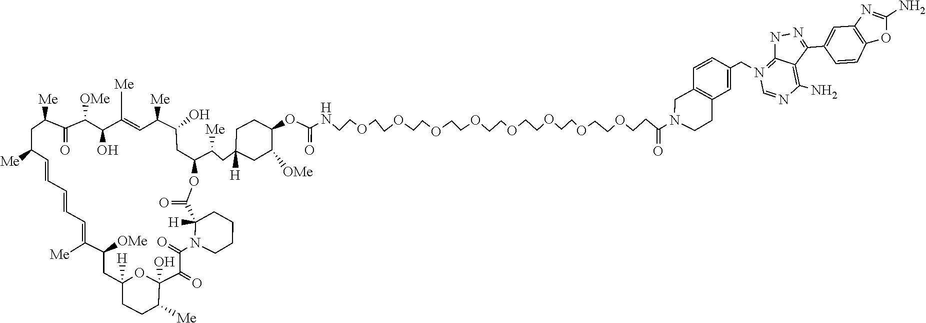 | C₉₃H₁₃₆N₁₀O₂₄ | [M + H] = 1777.98 | [M + H] = 1777.7 |
| Example 9 | | | |
| 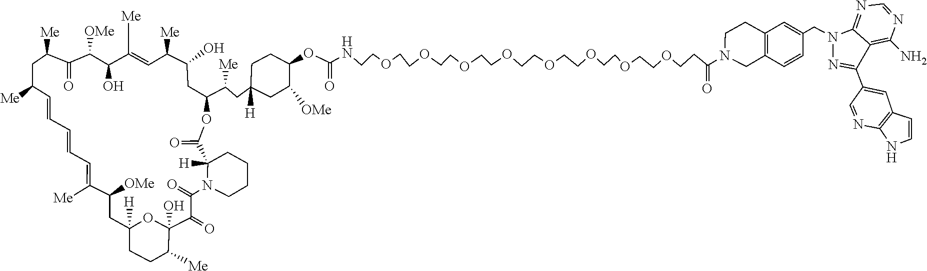 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₃H₁₃₆N₁₀O₂₃ | [M + H] = 1761.99 | [M + H] = 1761.7 |
| Example 10 | | | |
| 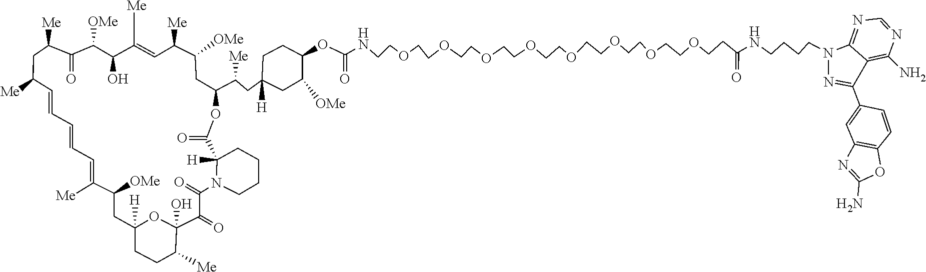 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4ccc5oc(N)nc5c4)c4c(N)ncnc43)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₈₈H₁₃₆N₁₀O₂₄ | [M + H] = 1717.98 | [M + H] = 1718.0 |
| Example 11 | | | |
| 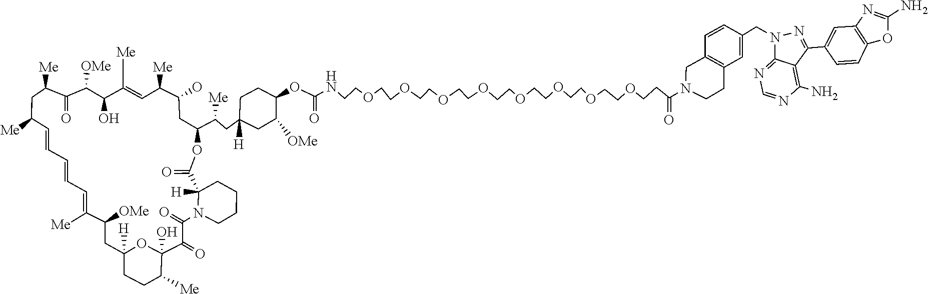 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₄H₁₃₈N₁₀O₂₄ | [M + H] = 1792.00 | [M + H] = 1791.9 |
| Example 12 | | | |
| 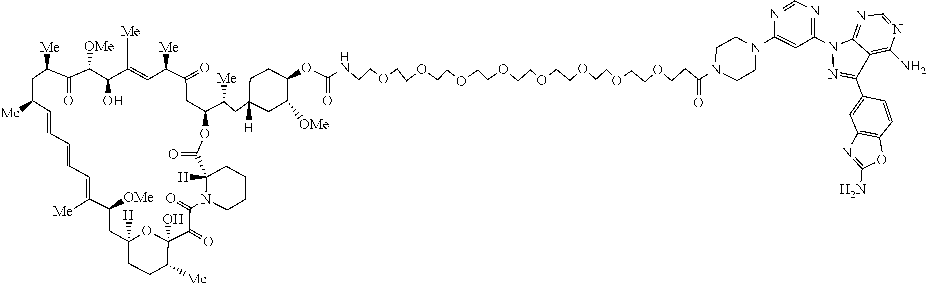 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCN(c4cc(-n5nc(-c6ccc7oc(N)nc7c6)c6c(N)ncnc65)ncn4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₃₃N₁₃O₂₄ | [M + H] = 1792.97 | [M + H] = 1793.0 |
| Example 13 | | | |
| 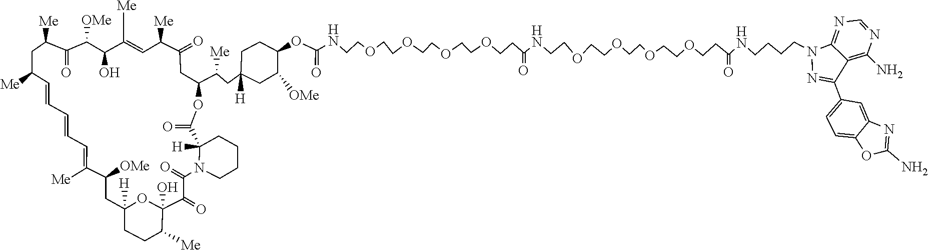 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)NCCCCn3nc(-c4ccc5oc(N)nc5c4)c4c(N)ncnc43)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₇N₁₁O₂₅ | [M + H] = 1772.99 | [M + H] = 1772.9 |
| Example 14 | | | |
| 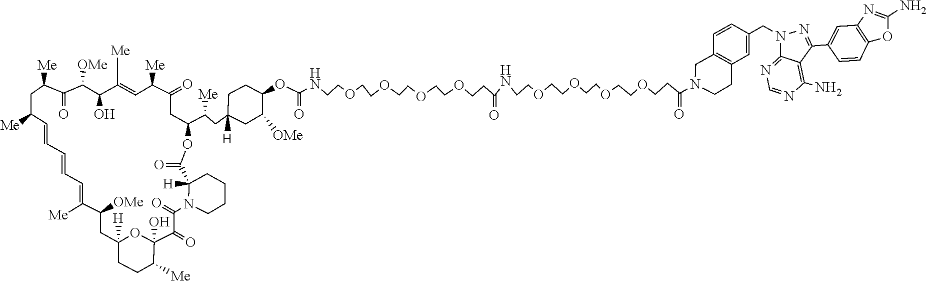 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₉N₁₁O₂₅ | [M + H] = 1847.00 | [M + H] = 1847.0 |
| Example 15 | | | |
| 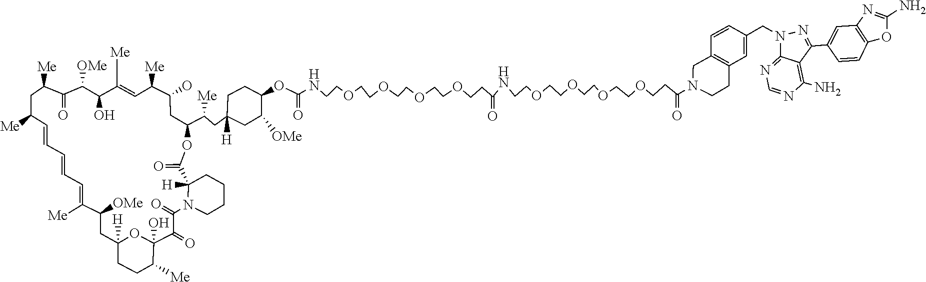 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₆H₁₄₁N₁₁O₂₅ | [M + H] = 1849.02 | [M + H] = 1848.9 |
| Example 16 | | | |
| 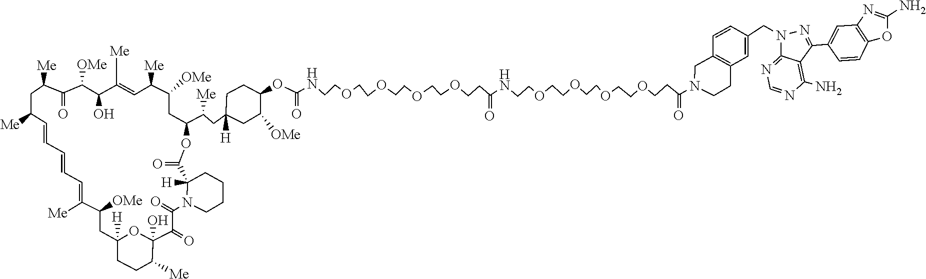 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₇H₁₄₃N₁₁O₂₅ | [M + H] = 1863.04 | [M + H] = 1863.0 |
| Example 17 | | | |
| 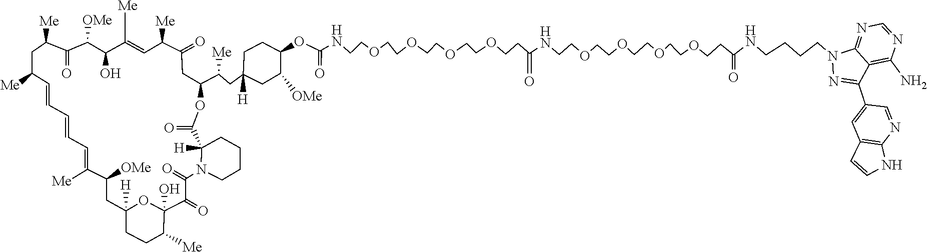 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCCOCCOCCOCCOCCC(=O)NCCCCn3nc(-c4cnc5[nH]ccc5c4)c4c(N)ncnc43)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₇N₁₁O₂₄ | [M + H] = 1756.99 | [M + H] = 1756.8 |
| Example 18 | | | |
| 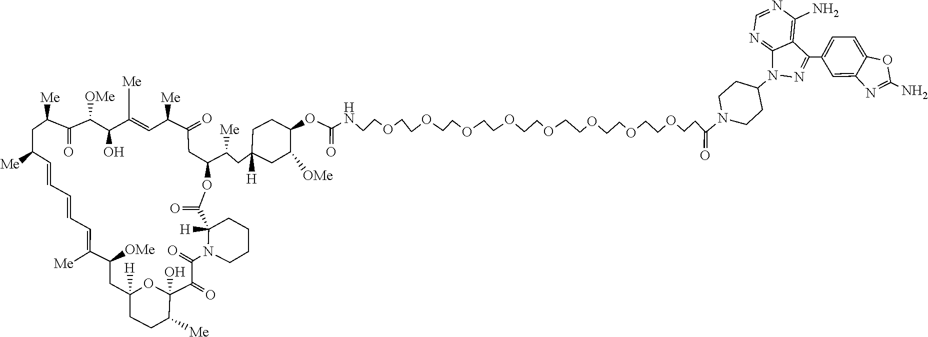 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCC(n4nc(-c5ccc6oc(N)nc6c5)c5c(N)ncnc54)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₈H₁₃₂N₁₀O₂₄ | [M + H] = 1713.95 | [M + H] = 1713.9 |
| Example 19 | | | |
| 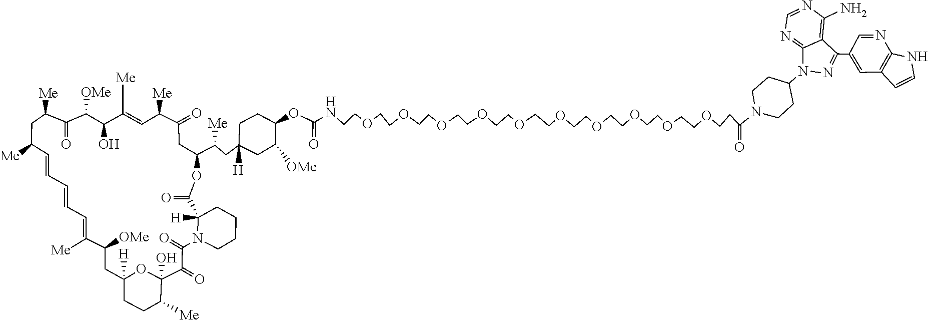 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCC(=O)N3CCC(n4nc(-c5cnc6[nH]ccc6c5)c5c(N)ncnc54)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₄₀N₁₀O₂₅ | [M + H] = 1786.01 | [M + H] = 1786.0 |
| Example 20 | | | |
| 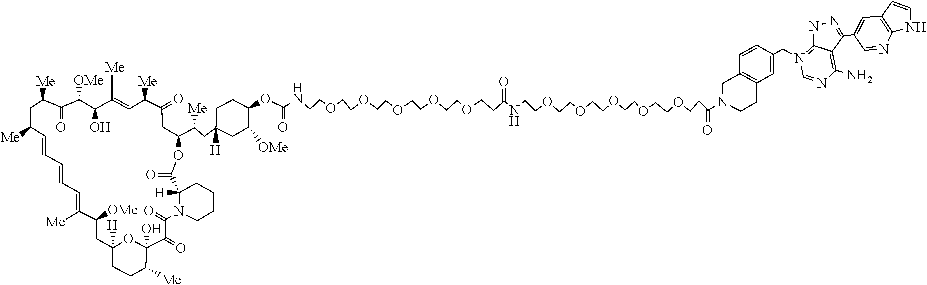 | C₁₀₀H₁₄₇N₁₁O₂₆ | [M + H] = 1919.06 | [M + H] = 1919.0 |
| Example 21 | | | |
| 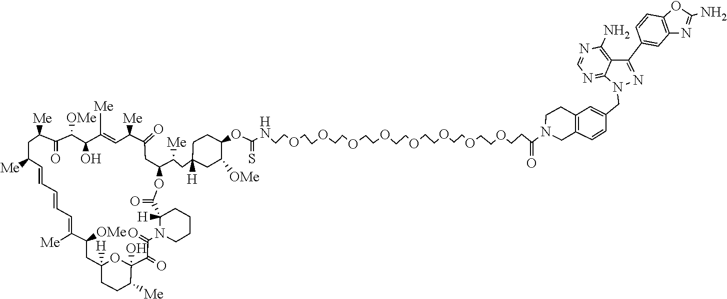 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₃₄N₁₀O₂₃S | [M + H] = 1791.94 | [M + H] = 1791.8 |
| Example 22 | | | |
| 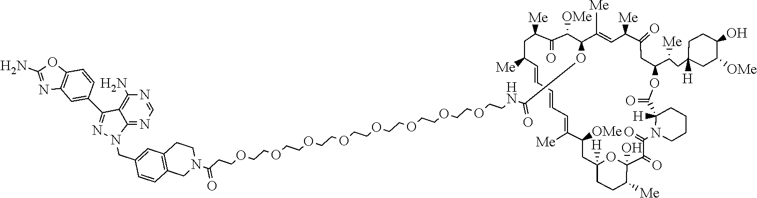 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](OC(=O)NCCOCCOCCOCCOCCOCCOCCOCCOCCC(=O)N2CCc3cc(Cn4nc(-c5ccc6oc(N)nc6c5)c5c(N)ncnc54)ccc3C2)[C@@H](OC)C(=O)[C@H](C)C[C@H](C)/C=C/C=C/C=C/1C] | C₉₃H₁₃₄N₁₀O₂₄ | [M + H] = 1775.97 | [M + H] = 1775.9 |
| Example 23 | | | |
| 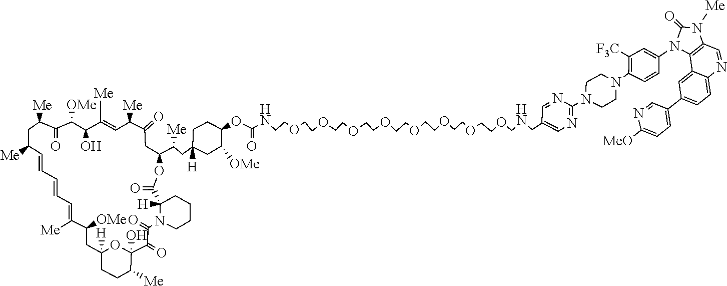 [SMILES: COc1ccc(-c2ccc3ncc4c(c3c2)n(-c2ccc(N3CCN(c5ncc(CNCOCCOCCOCCOCCOCCOCCOCCOCCNC(=O)O[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)cn5)CC3)c(C(F)(F)F)c2)c(=O)n4C)cn1] | C₁₀₄H₁₄₄F₃N₁₁O₂₅ | [M + 2H]/2 =[ 1003.03 | [M + 2H]/2 =[ 1003.5 |
| Example 24 | | | |
| 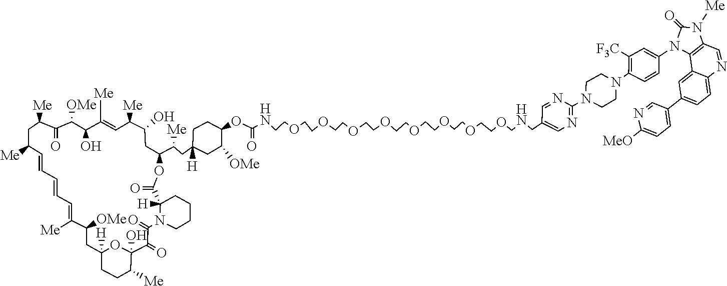 [SMILES: COc1ccc(-c2ccc3ncc4c(c3c2)n(-c2ccc(N3CCN(c5ncc(CNCOCCOCCOCCOCCOCCOCCOCCOCCNC(=O)O[C@@H]6CC[C@@H](C[C@@H](C)[C@@H]7C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)cn5)CC3)c(C(F)(F)F)c2)c(=O)n4C)cn1] | C₁₀₄H₁₄₆F₃N₁₁O₂₅ | [M + 2H]/2 =[ 1004.03 | [M + 2H]/2 =[ 1004.5 |
| Example 25 | | | |
| 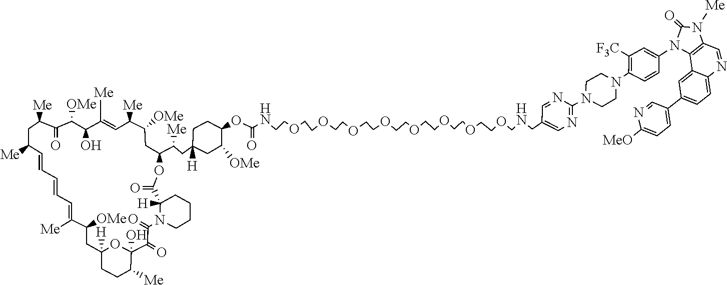 [SMILES: COc1ccc(-c2ccc3ncc4c(c3c2)n(-c2ccc(N3CCN(c5ncc(CNCOCCOCCOCCOCCOCCOCCOCCOCCNC(=O)O[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)cn5)CC3)c(C(F)(F)F)c2)c(=O)n4C)cn1] | C₁₀₅H₁₄₈F₃N₁₁O₂₅ | [M + 2H]/2 =[ 1011.04 | [M + 2H]/2 =[ 1011.5 |
| Example 26 | | | |
| 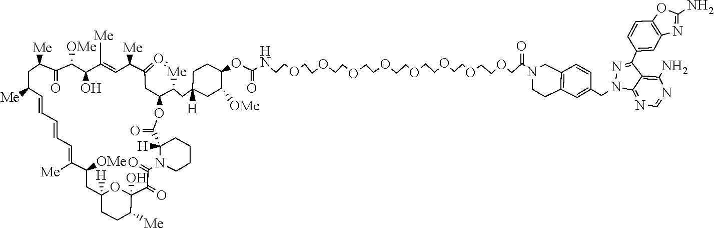 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCC(=O)N3CCc4cc(Cn5nc(-c6ccc7oc(N)nc7c6)c6c(N)ncnc65)ccc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₂N₁₀O₂₄ | [M + H] = 1761.95 | [M + H] = 1761.8 |
| Example 71 | | | |
| 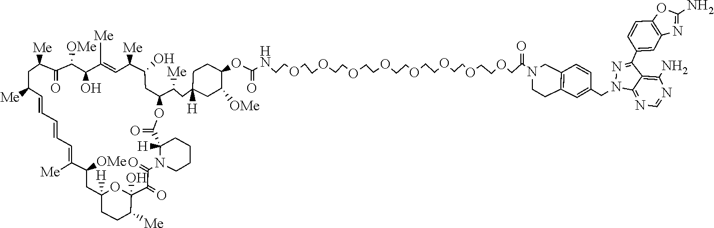 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₂H₁₃₄N₁₀O₂₄ | [M + H] = 1763.97 | [M + H] = 1763.8 |
| Example 72 | | | |
| 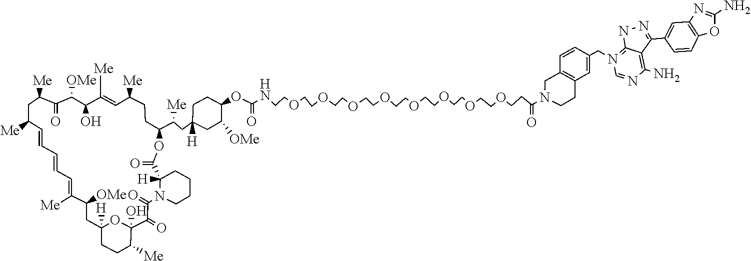 | C₉₃H₁₃₆N₁₀O₂₃ | [M + H] = 1761.99 | [M + H] = 1762.0 |
| Example 73 | | | |
| 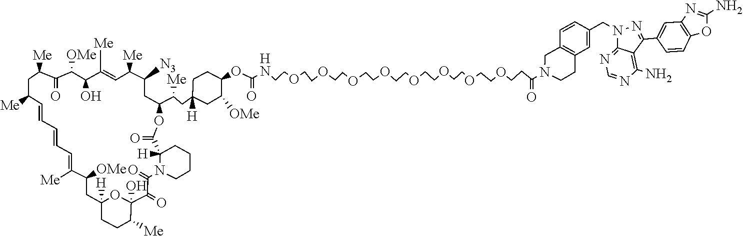 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] | C₉₃H₁₃₅N₁₃O₂₃ | [M + H] = 1802.99 | [M + H] = 1802.9 |
| Example 74 | | | |
| 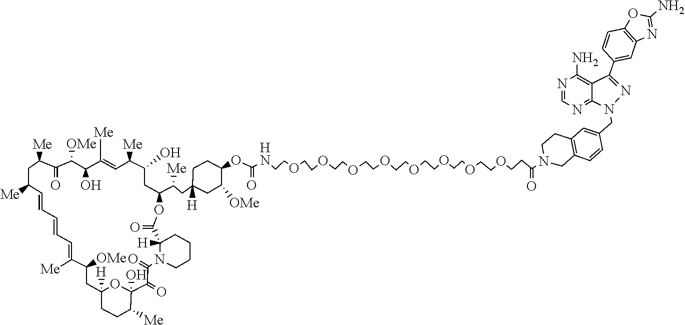 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₄H₁₃₈N₁₀O₂₄ | [M + H] = 1792.00 | [M + H] = 1791.7 |
| Example 75 | | | |
| 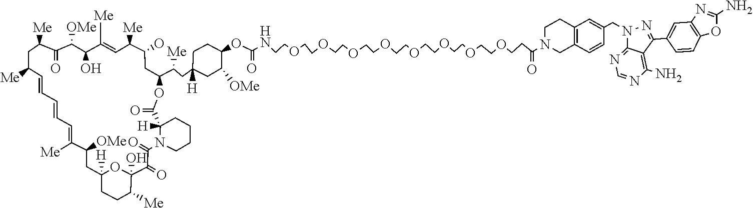 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₀N₁₀O₂₄ | [M + H] = 1806.01 | [M + H] = 1805.8 |
| Example 76 | | | |
| 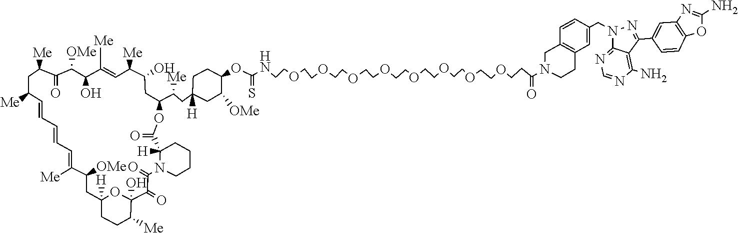 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₃H₁₃₆N₁₀O₂₃S | [M + H] = 1793.96 | [M + H] = 1793.9 |
| Example 77 | | | |
| 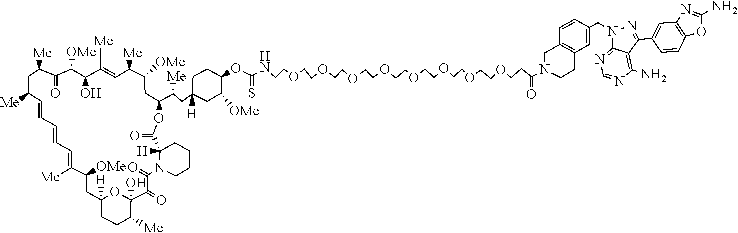 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₄H₁₃₈N₁₀O₂₃S | [M + H] = 1807.97 | [M + H] = 1807.9 |
| Example 78 | | | |
| 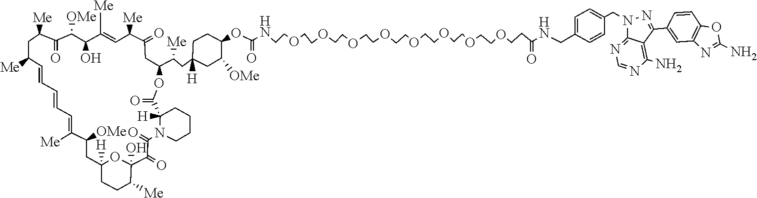 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₃₂N₁₀O₂₄ | [M + H] = 1749.95 | [M + H] = 1749.9 |
| Example 79 | | | |
| 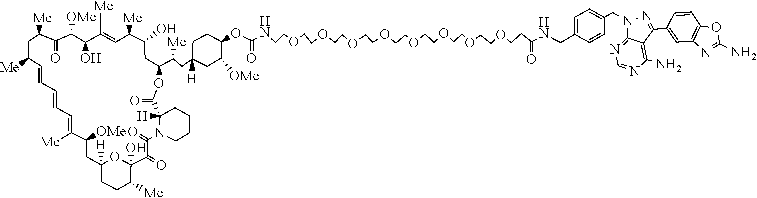 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₁H₁₃₄N₁₀O₂₄ | [M + H] = 1751.97 | [M + H] = 1751.9 |
| Example 80 | | | |
| 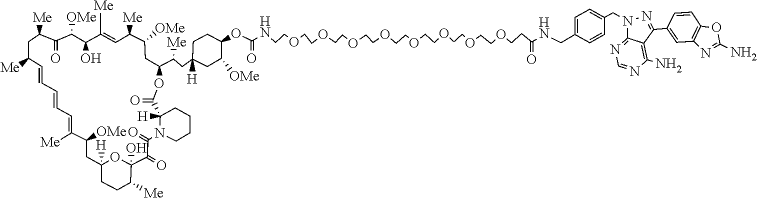 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₂H₁₃₆N₁₀O₂₄ | [M + H] = 1765.98 | [M + H] = 1765.8 |
| Example 81 | | | |
| 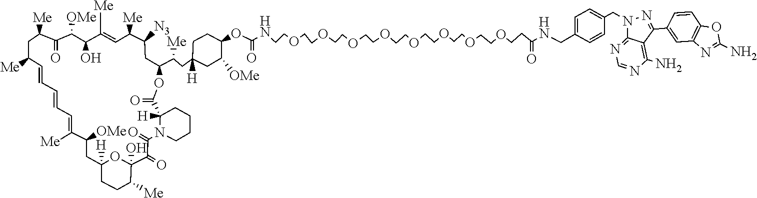 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] | C₉₁H₁₃₃N₁₃O₂₃ | [M + H] = 1776.97 | [M + H] = 1776.9 |
| Example 82 | | | |
| 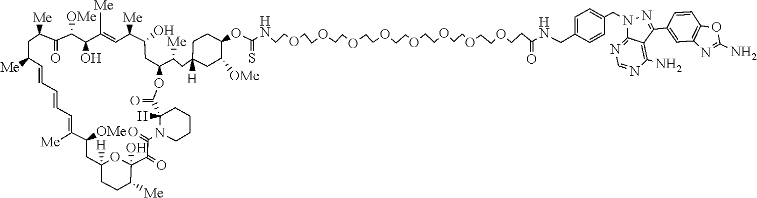 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCc3ccc(Cn4nc(-c5ccc6oc(N)nc6c5)c5c(N)ncnc54)cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₁H₁₃₃N₁₀O₂₃S | [M + H] = 1766.93 | [M + H] = 1766.8 |
| Example 83 | | | |
| 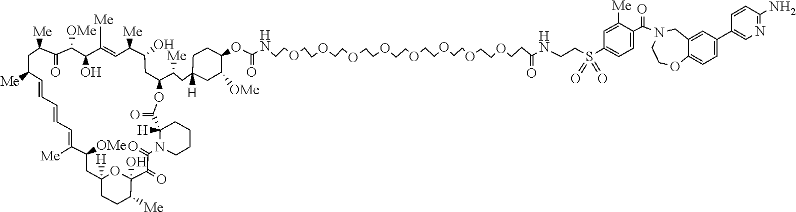 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₀N₆O₂₇S | [M + H] = 1829.96 | [M + H] = 1830.0 |
| Example 84 | | | |
| 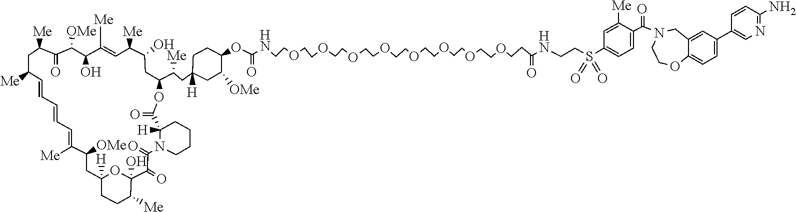 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₂N₆O₂₇S | [M + H] = 1831.97 | [M + H] = 1831.9 |
| Example 85 | | | |
| 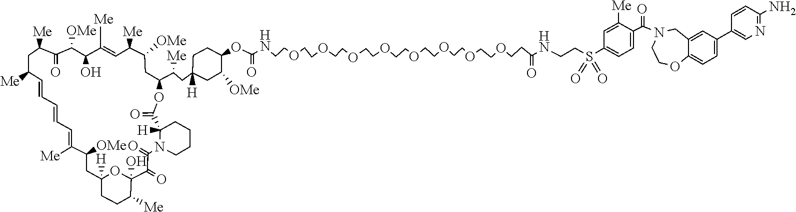 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₆H₁₄₄N₆O₂₇S | [M + H] = 1845.99 | [M + H] = 1846.0 |
| Example 86 | | | |
| 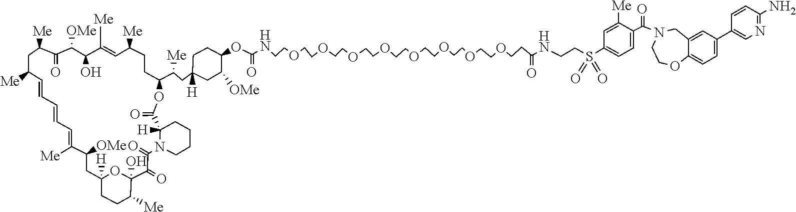 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)CC[C@H](C)/C=C(\C)[C@@H](O)[C@@H](OC)C(=O)[C@H](C)C[C@H](C)/C=C/C=C/C=C/1C] | C₉₅H₁₄₂N₆O₂₆S | [M + H] = 1815.98 | [M + H] = 1815.8 |
| Example 87 | | | |
| 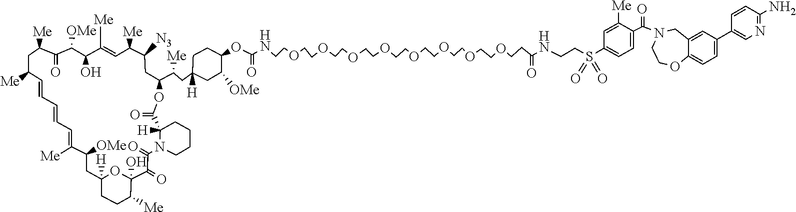 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] | C₉₅H₁₄₁N₉O₂₆S | [M + H] = 1856.98 | [M + H] = 1856.8 |
| Example 88 | | | |
| 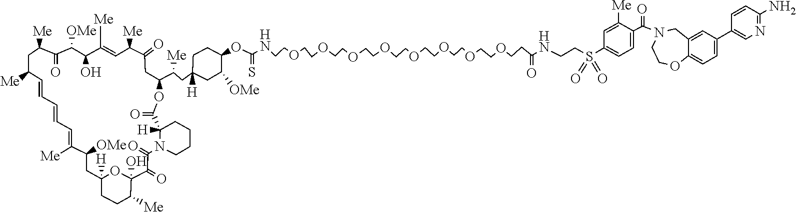 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₄₀N₆O₂₆S₂ | [M + H] = 1845.93 | [M + H] = 1846.0 |
| Example 89 | | | |
| 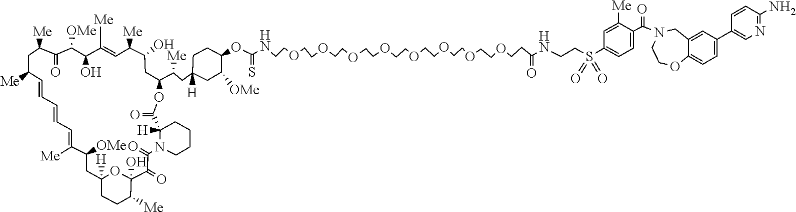 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₂N₆O₂₆S₂ | [M + H] = 1847.95 | [M + H] = 1847.9 |
| Example 90 | | | |
| 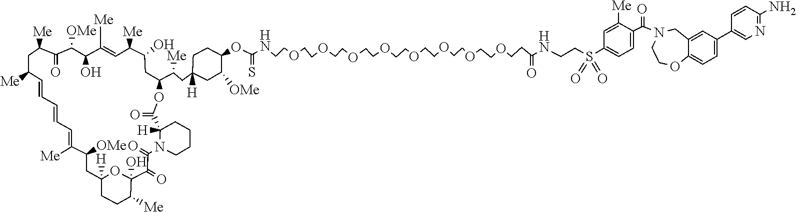 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₆H₁₄₄N₆O₂₆S₂ | [M + H] = 1861.96 | [M + H] = 1861.7 |
| Example 91 | | | |
| 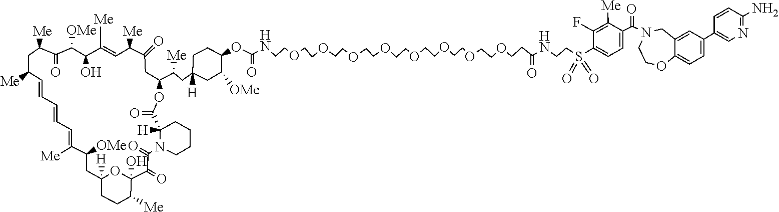 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₉FN₆O₂₇S | [M + H] = 1847.95 | [M + H] = 1848.0 |
| Example 92 | | | |
| 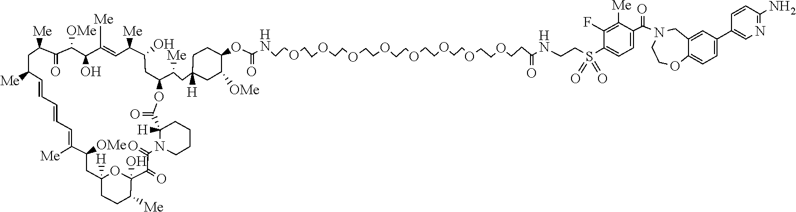 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₁FN₆O₂₇S | [M + H] = 1849.96 | [M + H] = 1850.0 |
| Example 93 | | | |
| 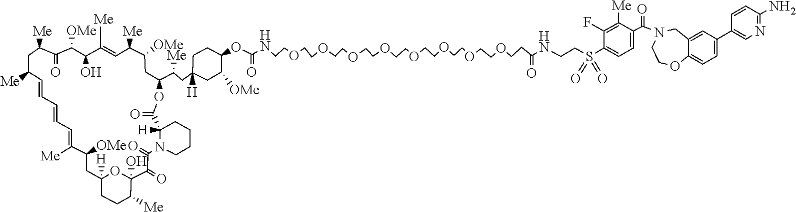 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₆H₁₄₃FN₆O₂₇S | [M + H] = 1863.98 | [M + H] = 1864.0 |
| Example 94 | | | |
| 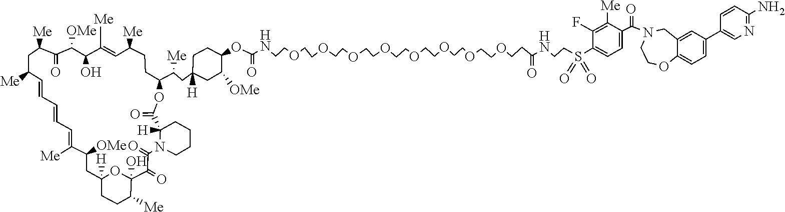 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)CC[C@H](C)/C=C(\C)[C@@H](O)[C@@H](OC)C(=O)[C@H](C)C[C@H](C)/C=C/C=C/C=C/1C] | C₉₆H₁₄₃FN₆O₂₆S | [M + H] = 1833.97 | [M + H] = 1833.9 |
| Example 95 | | | |
| 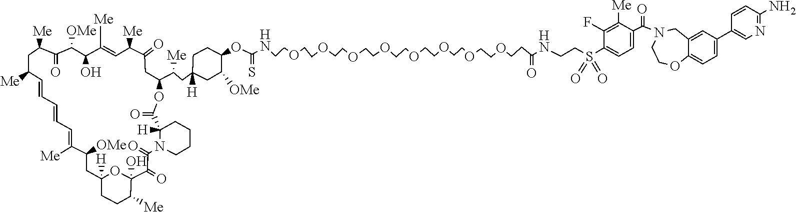 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₉FN₆O₂₆S₂ | [M + H] = 1863.92 | [M + H] = 1863.8 |
| Example 96 | | | |
| 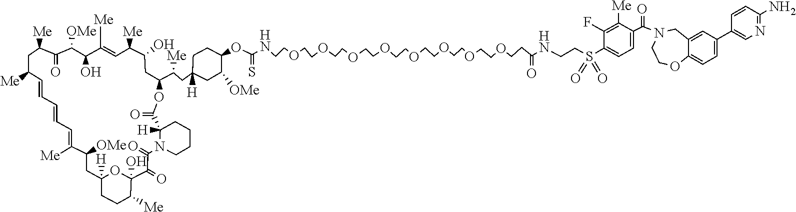 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₁FN₆O₂₆S₂ | [M + H] = 1865.94 | [M + H] = 1865.8 |
| Example 97 | | | |
| 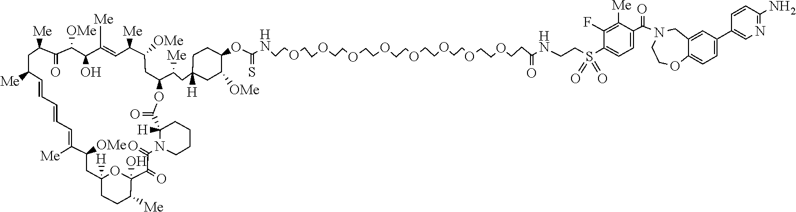 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCCOCCOCCOCCOCCOCCOCCOCCOCCC(=O)NCCS(=O)(=O)c3ccc(C(=O)N4CCOc5ccc(-c6ccc(N)nc6)cc5C4)c(C)c3F)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₆H₁₄₃FN₆O₂₆S₂ | [M + H] = 1879.96 | [M + H] = 1879.9 |
| Example 98 | | | |
| 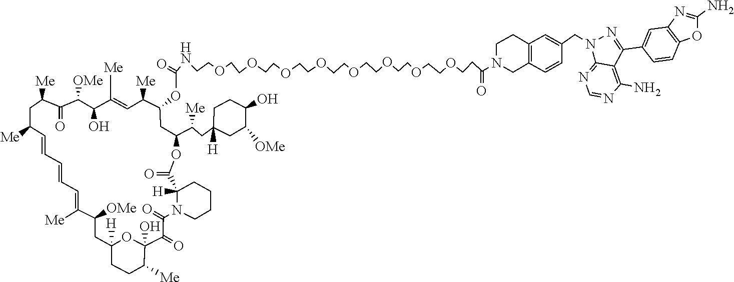 | C₉₃H₁₃₆N₁₀O₂₄ | [M + H] = 1777.98 | [M + H] = 1777.8 |
| Example 99 | | | |
| 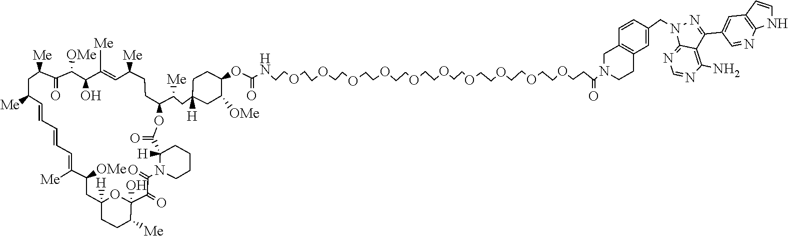 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)CC[C@H](C)/C=C(\C)[C@@H](O)[C@@H](OC)C(=O)[C@H](C)C[C@H](C)/C=C/C=C/C=C/1C] | C₉₇H₁₄₄N₁₀O₂₄ | [M + H] = 1834.04 | [M + H] = 1833.8 |
| Example 100 | | | |
| 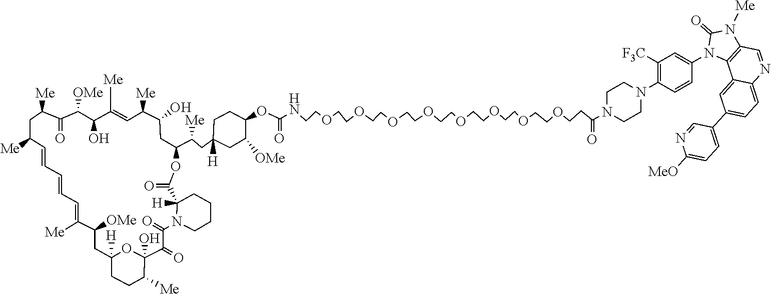 [SMILES: COc1ccc(-c2ccc3ncc4c(c3c2)n(-c2ccc(N3CCN(C(=O)CCOCCOCCOCCOCCOCCOCCOCCOCCNC(=O)O[C@@H]5CC[C@@H](C[C@@H](C)[C@@H]6C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC3)c(C(F)(F)F)c2)c(=O)n4C)cn1] | C₉₉H₁₄₁F₃N₈O₂₅ | [M + H] = 1900.00 | [M + H] = 899.9 |
| Example 101 | | | |
| 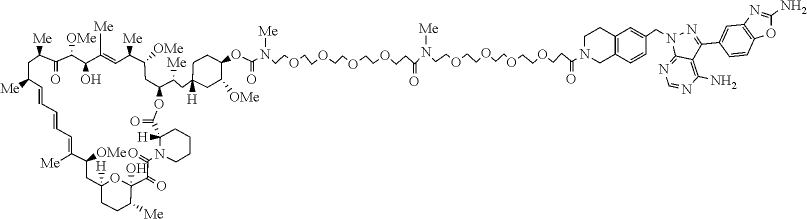 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)CCOCCOCCOCCOCCC(=O)N(C)CCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₉H₁₄₇N₁₁O₂₅ | [M + H] = 1891.06 | [M + H] = 1890.9 |
| Example 102 | | | |
| 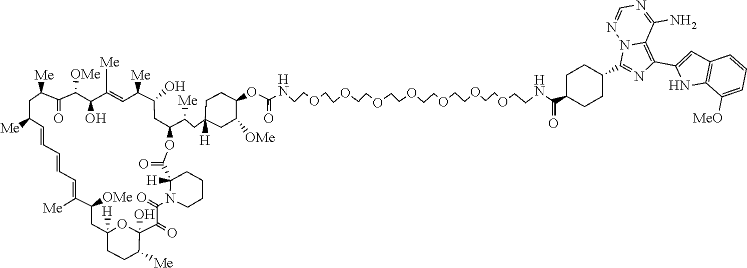 [SMILES: COc1cccc2cc(-c3nc([C@H]4CC[C@H](C(=O)NCCOCCOCCOCCOCCOCCOCCOCCNC(=O)O[C@@H]5CC[C@@H](C[C@@H](C)[C@@H]6C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] | C₈₉H₁₃₅N₉O₂₃ | [M + H] = 1698.97 | [M + H] = 1698.81 |
| Example 136 | | | |
| 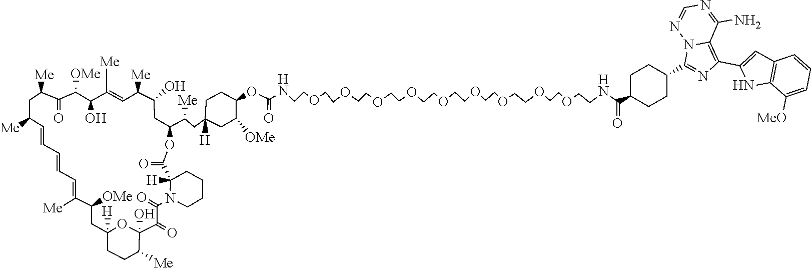 [SMILES: COc1cccc2cc(-c3nc([C@H]4CC[C@H](C(=O)NCCOCCOCCOCCOCCOCCOCCOCCOCCOCCNC(=O)O[C@@H]5CC[C@@H](C[C@@H](C)[C@@H]6C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] | C₉₃H₁₄₃N₉O₂₅ | [M + H] = 1787.03 | [M + H] = 1786.7 |
| Example 137 | | | |
| 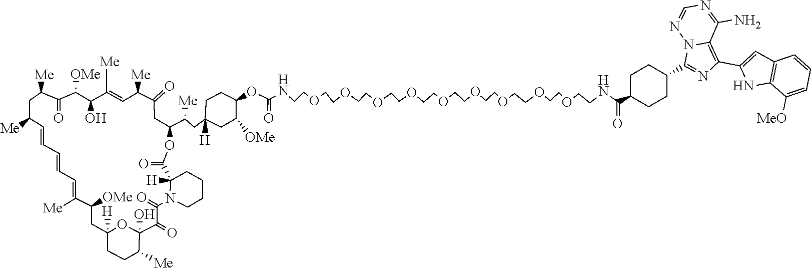 [SMILES: COc1cccc2cc(-c3nc([C@H]4CC[C@H](C(=O)NCCOCCOCCOCCOCCOCCOCCOCCOCCOCCNC(=O)O[C@@H]5CC[C@@H](C[C@@H](C)[C@@H]6CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] | C₉₃H₁₄₁N₉O₂₅ | [M + H] = 1785.01 | [M + H] = 1784.9 |
| Example 138 | | | |
| 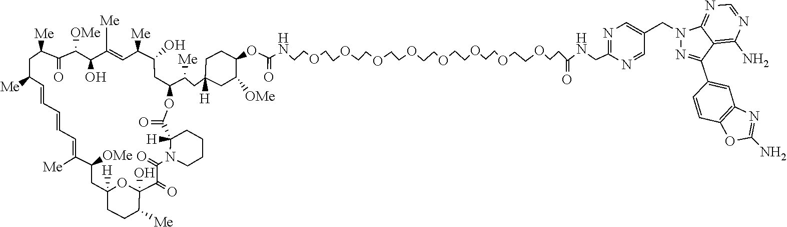 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCc3ncc(Cn4nc(-c5ccc6oc(N)nc6c5)c5c(N)ncnc54)cn3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₈₉H₁₃₂N₁₂O₂₄ | [M + H] = 1753.96 | [M + H] = 1753.7 |
| Example 139 | | | |
| 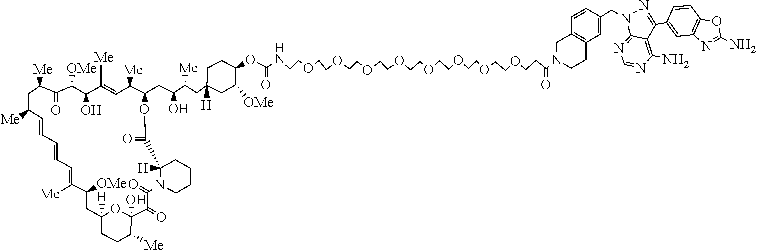 [SMILES: CO[C@H]1C[C@@H]2CC[C@@H](C)[C@@](O)(O2)C(=O)C(=O)N2CCCC[C@H]2C(=O)O[C@H](C[C@H](O)[C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)[C@H](C)/C=C(\C)[C@@H](O)[C@@H](OC)C(=O)[C@H](C)C[C@H](C)/C=C/C=C/C=C/1C] | C₉₃H₁₃₆N₁₀O₂₄ | [M + H] = 1777.98 | [M + H] = 1777.7 |
| Example 140 | | | |
| 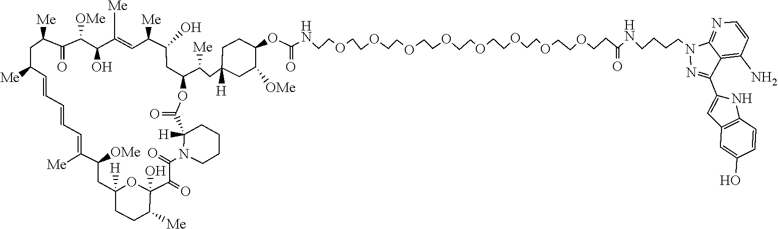 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCCCn3nc(-c4cc5cc(O)ccc5[nH]4)c4c(N)ccnc43)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₈₈H₁₃₅N₉O₂₄ | [M + H] = 1702.97 | [M + H] = 1702.9 |
| Example 141 | | | |
| 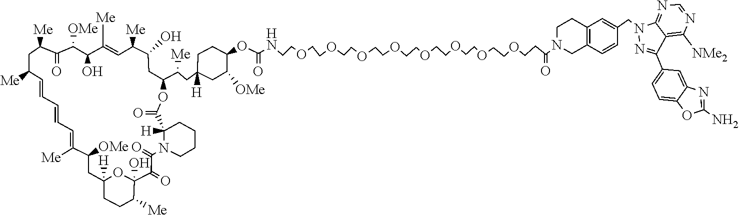 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N(C)C)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₀N₁₀O₂₄ | [M + H] = 1806.01 | [M + H] = 1805.8 |
| Example 142 | | | |
| 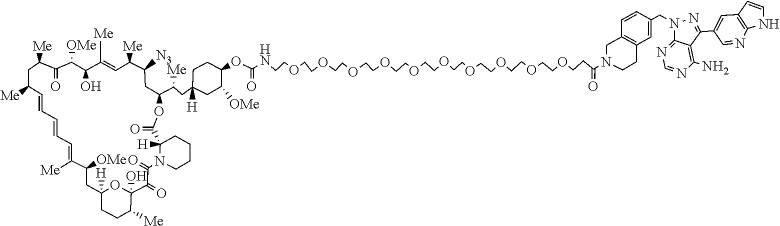 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCOCCOCCC(=O)N3CCc4cc(Cn5nc(-c6cnc7[nH]ccc7c6)c6c(N)ncnc65)ccc4C3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] | C₉₇H₁₄₃N₁₃O₂₄ | [M + H] = 1875.04 | [M + H] = 1874.9 |
| Example 143 | | | |
| 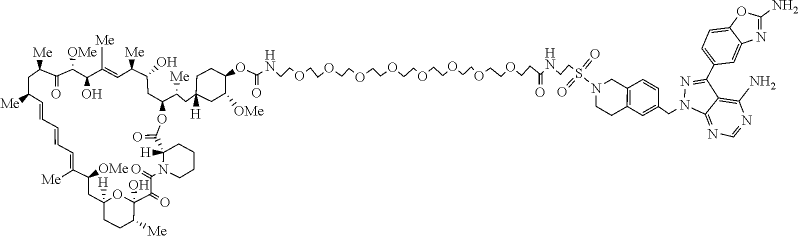 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)NCCS(=O)(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₄₁N₁₁O₂₆S | [M + H] = 1884.98 | [M + H] = 1884.9 |
| Example 144 | | | |
| 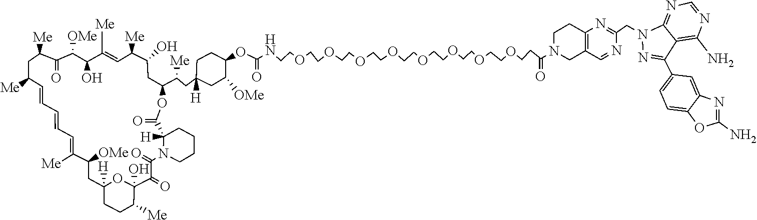 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4nc(Cn5nc(-c6ccc7oc(N)nc7c6)c6c(N)ncnc65)ncc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₁H₁₃₄N₁₂O₂₄ | [M + H] = 1779.97 | [M + H] = 1779.8 |
| Example 145 | | | |
| 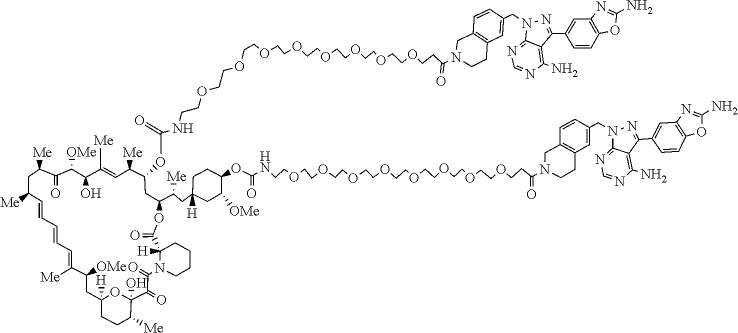 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCOCCOCCC(=O)N3CCc4cc(Cn5nc(-c6ccc7oc(N)nc7c6)c6c(N)ncnc65)ccc4C3)[C@H](OC)C2)C[C@@H](OC(=O)NCCOCCOCCOCCOCCOCCOCCOCCOCCC(=O)N2CCc3cc(Cn4nc(-c5ccc6oc(N)nc6c5)c5c(N)ncnc54)ccc3C2)[C@H](C)/C=C(\C)[C@@H](O)[C@@H](OC)C(=O)[C@H](C)C[C@H](C)/C=C/C=C/C=C/1C] | C₁₃₅H₁₉₁N₁₉O₃₅ | [M + 2H]/2 =[ 1320.19 | [M + 2H]/2 =[ 1320.8 |
| Example 146 | | | |
| 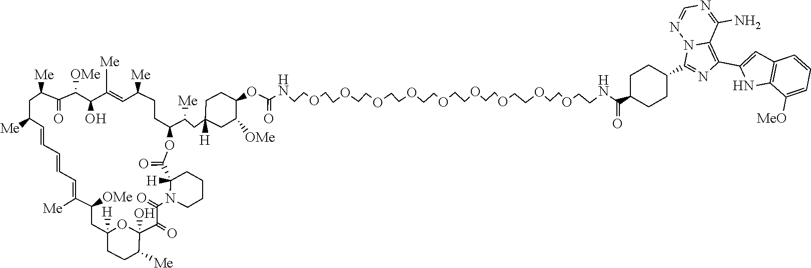 [SMILES: COc1cccc2cc(-c3nc([C@H]4CC[C@H](C(=O)NCCOCCOCCOCCOCCOCCOCCOCCOCCOCCNC(=O)O[C@@H]5CC[C@@H](C[C@@H](C)[C@@H]6CC[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] | C₉₃H₁₄₃N₉O₂₄ | [M + H] = 1771.03 | [M + H] = 1770.8 |
| Example 147 | | | |
| 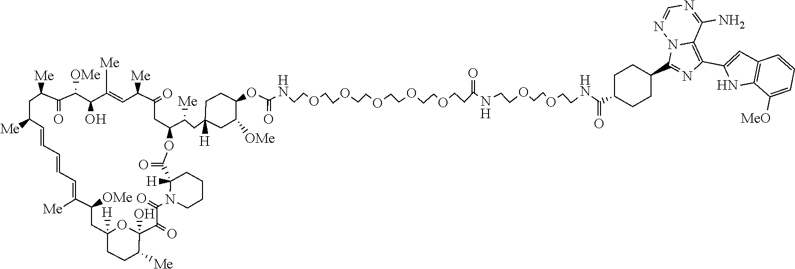 [SMILES: COc1cccc2cc(-c3nc([C@H]4CC[C@H](C(=O)NCCOCCOCCNC(=O)CCOCCOCCOCCOCCOCCNC(=O)O[C@@H]5CC[C@@H](C[C@@H](C)[C@@H]6CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O6)C[C@H]5OC)CC4)n4ncnc(N)c34)[nH]c12] | C₉₂H₁₃₈N₁₀O₂₄ | [M + H] = 1768.00 | [M + H] = 1767.8 |
| Example 148 | | | |

### General Procedure 3: Coupling of a Halide Containing PEG Ester and an Amine Containing Pre-Linker Followed by Ester Deprotection

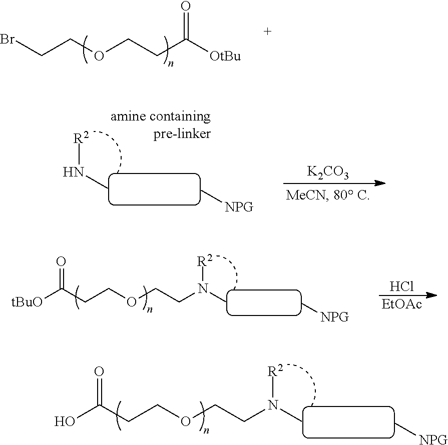

### Step 1

To a 0.1 M solution of amine containing pre-linker (1.0 equiv) in MeCN was added K₂CO₃ (2.0 equiv) followed by halide containing PEG ester (1.0 equiv). The reaction was stirred at 80° C. until consumption of amine containing pre-linker, as indicated by LCMS analysis. The reaction was then purified by silica gel chromatography to afford the product.

### Step 2

To a 0.1 M solution of PEG tert-butyl ester (1.0 equiv) in EtOAc was added a solution of HCl in EtOAc. The resulting suspension was stirred at room temperature until consumption of the PEG ester, as indicated by LCMS analysis. The reaction was then concentrated under reduced pressure to afford the product.

### Intermediate B1-1. 1-(4-(5-((1,3-dioxoisoindolin-2-yl)methyl)pyrimidin-2-yl)piperazin-1-yl)-3,6,9,12-tetraoxapentadecan-15-oic acid

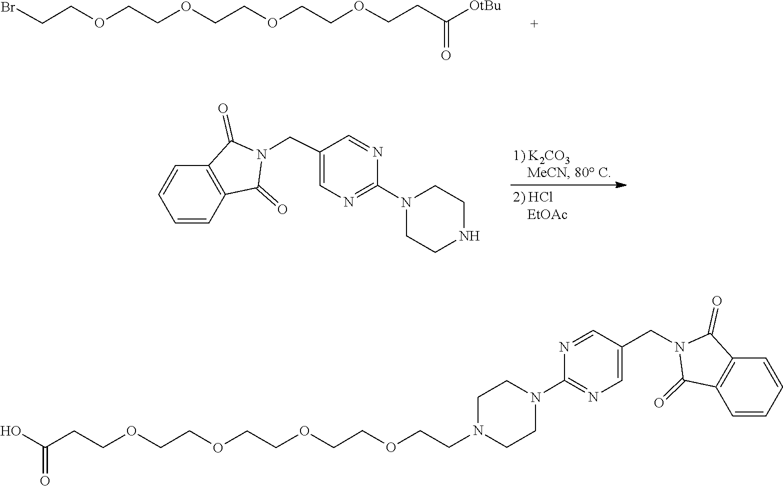 [SMILES: CC(C)(C)OC(=O)CCOCCOCCOCCOCCBr ; O=C1c2ccccc2C(=O)N1Cc1cnc(N2CCNCC2)nc1 ; O=C(O)CCOCCOCCOCCOCCN1CCN(c2ncc(CN3C(=O)c4ccccc4C3=O)cn2)CC1]

### Step 1: Synthesis of tert-butyl 1-(4-(5-((1,3-dioxoisoindolin-2-yl)methyl)pyrimidin-2-yl)piperazin-1-yl)-3,6,9,12-tetraoxapentadecan-15-oate

To a mixture of 2-((2-(piperazin-1-yl)pyrimidin-5-yl)methyl)isoindoline-1,3-dione (7.97 g, 24.66 mmol, 1.0 equiv) in MeCN (200 mL) was added K₂CO₃ (6.82 g, 49.31 mmol, 2.0 equiv) followed by tert-butyl 1-bromo-3,6,9,12-tetraoxapentadecan-15-oate (9.5 g, 24.66 mmol, 1.0 equiv). The reaction mixture was heated to 85° C. and stirred for 15 h. The mixture was then cooled to room temperature and filtered. The filtrate was concentrated under reduced pressure and the residue was purified by silica gel chromatography (0→20% EtOAc/MeOH) to give the product (11.5 g, 74.3% yield) a light yellow liquid.

### Step 2: Synthesis of 1-(4-(5-((1,3-dioxoisoindolin-2-yl)methyl)pyrimidin-2-yl)piperazin-1-yl)-3,6,9,12-tetraoxapentadecan-15-oic acid

To a solution of tert-butyl 1-(4-(5-((1,3-dioxoisoindolin-2-yl)methyl)pyrimidin-2-yl)piperazin-1-yl)-3,6,9,12-tetraoxapentadecan-15-oate (3.5 g, 5.58 mmol, 1.0 equiv) in EtOAc (50 mL) was added a solution of HCl in EtOAc (500 mL). The mixture was stirred at room temperature for 3 h. The mixture was then concentrated under reduced pressure to give the product (5.3 g, 78.2% yield, HCl) as a white solid. LCMS (ESI) m/z: [M+H] calcd for C₂₈H₃₇N₅O₈: 572.27; found 572.4.

Following General Procedure 3, but using the appropriate halide containing PEG and amine containing pre-linkers in Table 4, the Intermediates B1 in Table 8 were prepared:

TABLE 8
Additional protected amines prepared

| | Molecular | Calculated | Observed |
|---|---|---|---|
| Structure | Formula | MW | MW |
| 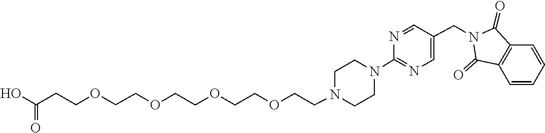 [SMILES: O=C(O)CCOCCOCCOCCOCCN1CCN(c2ncc(CN3C(=O)c4ccccc4C3=O)cn2)CC1] | C₂₈H₃₇N₅O₈ | [M + H] = 572.27 | [M + H] = 572.4 |
| Intermediate B1-1 | | | |

### General Procedure 4: Coupling of a PEG Carboxylic Acid and an Amine Containing Active Site Inhibitor Followed by Amine Deprotection

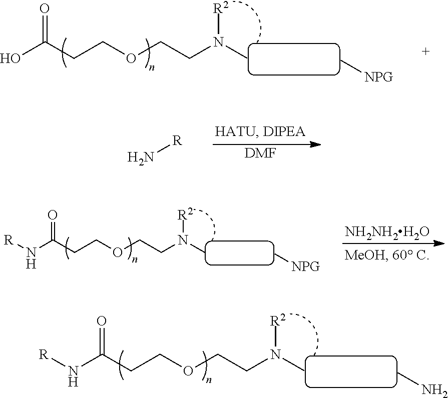

### Step 1

To a 0.15 M solution of PEG carboxylic acid (1.0 equiv) in DMF was added HATU (1.3 equiv) and DIPEA (5.0 equiv). After stirring for 30 min, the amine containing active site inhibitor (1.2 equiv) was added. The reaction was stirred at room temperature until consumption of PEG carboxylic acid, as indicated by LCMS. The reaction was then purified by reverse phase chromatography to afford the product.

### Step 2

To a 0.1 M solution of phthalimide protected amine (1.0 equiv) in MeOH at 0° C. was added NH₂NH₂·H₂O (4.0 equiv). The resulting mixture was stirred at 60° C. until consumption of the phthalimide protected amine, as indicated by LCMS analysis. The reaction was then purified by reverse phase chromatography to afford the product.

### Intermediate B2-1. N-(4-(4-amino-3-(2-aminobenzo[d]oxazol-5-yl)-1H-pyrazolo[3,4-d]pyrimidin-1-yl)butyl)-1-(4-(5-(aminomethyl)pyrimidin-2-yl)piperazin-1-yl)-3,6,9,12-tetraoxapentadecan-15-amide

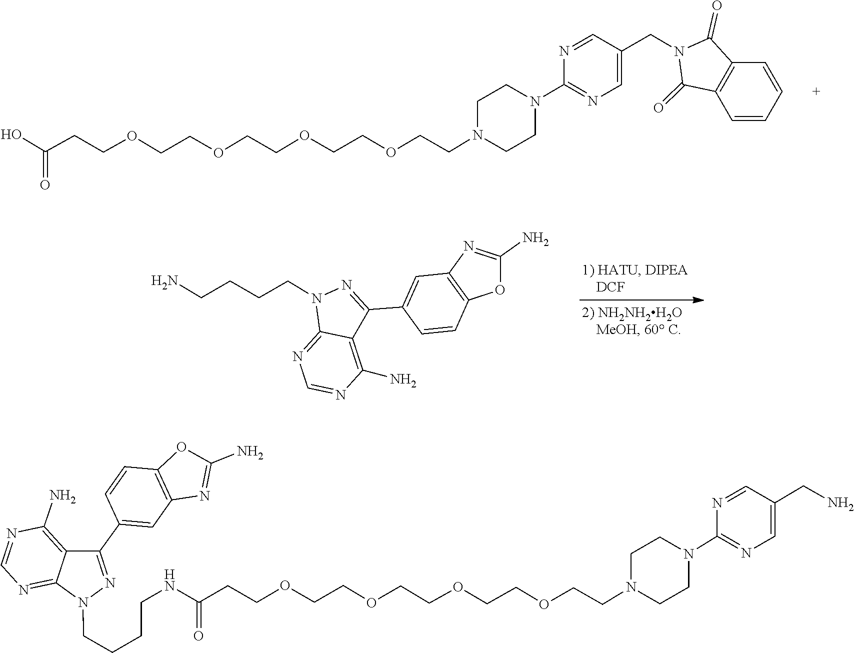 [SMILES: O=C(O)CCOCCOCCOCCOCCN1CCN(c2ncc(CN3C(=O)c4ccccc4C3=O)cn2)CC1 ; NCCCCn1nc(-c2ccc3oc(N)nc3c2)c2c(N)ncnc21 ; NCc1cnc(N2CCN(CCOCCOCCOCCOCCC(=O)NCCCCn3nc(-c4ccc5oc(N)nc5c4)c4c(N)ncnc43)CC2)nc1]

### Step 1: Synthesis of N-(4-(4-amino-3-(2-aminobenzo[d]oxazol-5-yl)-1H-pyrazolo[3,4-d]pyrimidin-1-yl)butyl)-1-(4-(5-((1,3-dioxoisoindolin-2-yl)methyl)pyrimidin-2-yl)piperazin-1-yl)-3,6,9,12-tetraoxapentadecan-15-amide

To a mixture of 1-(4-(5-((1,3-dioxoisoindolin-2-yl)methyl)pyrimidin-2-yl)piperazin-1-yl)-3,6,9,12-tetraoxapentadecan-15-oic acid (3 g, 4.93 mmol, 1.0 equiv, HCl) in DMF (30 mL) was added HATU (12.11 μL, 6.41 mmol, 1.3 equiv) and DIPEA (4.30 mL, 24.67 mmol, 5.0 equiv). After 30 min, 5-(4-amino-1-(4-aminobutyl)-1H-pyrazolo[3,4-d]pyrimidin-3-yl)benzo[d]oxazol-2-amine (4.03 g, 5.92 mmol, 1.2 equiv, 3TFA) was added. The mixture was stirred at room temperature for 3 h. The reaction mixture was then purified by prep-HPLC (MeCN/H₂O) to give the product (5.4 g, 81.2% yield, 4TFA) as a light red solid. LCMS (ESI) m/z: [M+2H]/2 calcd for C₄₄H₅₃N₁₃O₈: 446.71; found 447.0.

### Step 2: Synthesis of N-(4-(4-amino-3-(2-aminobenzo[d]oxazol-5-yl)-1H-pyrazolo[3,4-d]pyrimidin-1-yl)butyl)-1-(4-(5-(aminomethyl)pyrimidin-2-yl)piperazin-1-yl)-3,6,9,12-tetraoxapentadecan-15-amide

To a mixture of N-(4-(4-amino-3-(2-aminobenzo[d]oxazol-5-yl)-1H-pyrazolo[3,4-d]pyrimidin-1-yl)butyl)-1-(4-(5-((1,3-dioxoisoindolin-2-yl)methyl)pyrimidin-2-yl)piperazin-1-yl)-3,6,9,12-tetraoxapentadecan-15-amide (4 g, 2.97 mmol, 1.0 equiv, 4TFA) in MeOH (25 mL) at 0° C. was added NH₂NH₂·H₂O (588.63 μL, 11.87 mmol, 4.0 equiv). The mixture was stirred at 60° C. for 2 h. The mixture was then cooled to room temperature and filtered, and the filter cake was washed with MeOH (5 mL). The filtrate was concentrated under reduced pressure and the residue was purified by prep-HPLC (MeCN/H₂O) to give the product (700 mg, 24.50 yield, TFA) as a white solid. LCMS (ESI) m/z: [M+2H]/2 calcd for C₃₆H₅₁N₁₃O₆: 381.71; found 381.8.

Following General Procedure 4, but using the appropriate Intermediate B1 in Table 8 and amine containing active site inhibitors in Table 2, the Intermediates B2 in Table 9 were prepared:

TABLE 9
Additional amines prepared

| | Molecular | Calculated | Observed |
|---|---|---|---|
| Structure | Formula | MW | MW |
| 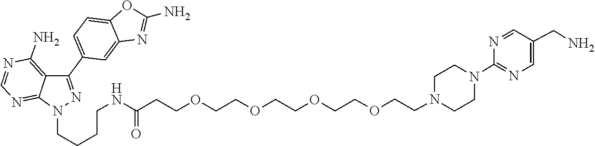 [SMILES: NCc1cnc(N2CCN(CCOCCOCCOCCOCCC(=O)NCCCCn3nc(-c4ccc5oc(N)nc5c4)c4c(N)ncnc43)CC2)nc1] | C₃₆H₅₁N₁₃O₆ | [M + 2H]/ 2 = 381.71 | [M + 2H]/ 2 = 381.8 |
| Intermediate B2-1 | | | |
| 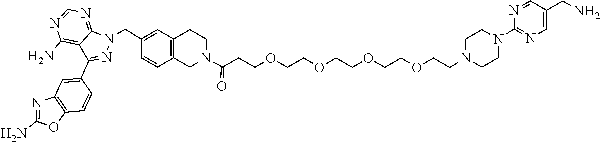 [SMILES: NCc1cnc(N2CCN(CCOCCOCCOCCOCCC(=O)N3CCc4cc(Cn5nc(-c6ccc7oc(N)nc7c6)c6c(N)ncnc65)ccc4C3)CC2)nc1] | C₄₂H₅₃N₁₃O₆ | [M + H] = 836.43 | [M + H] = 836.4 |
| Intermediate B2-2 | | | |
| 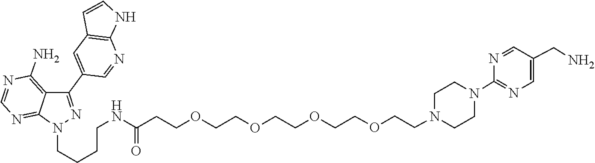 [SMILES: NCc1cnc(N2CCN(CCOCCOCCOCCOCCC(=O)NCCCCn3nc(-c4cnc5[nH]ccc5c4)c4c(N)ncnc43)CC2)nc1] | C₃₆H₅₁N₁₃O₅ | [M + 2H]/ 2 = 373.72 | [M + 2H]/ 2 = 737.7 |
| Intermediate B2-3 | | | |

### General Procedure 5: Coupling of a halide containing PEG carboxylic acid and an amine containing active site inhibitor

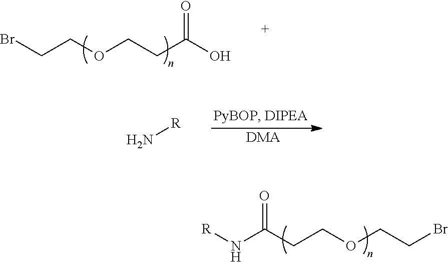

To a 0.1 M solution of amine containing active site inhibitor (1.0 equiv) and PEG containing carboxylic acid (1.2 equiv) in DMA was added DIPEA (4.0 equiv) followed by PyBOP (1.3 equiv). The reaction was stirred until consumption of amine containing active site inhibitor, as indicated by LCMS. The reaction was then purified by reverse phase HPLC to afford the product.

### Intermediate B3-1. 18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-1-bromo-3,6,9,12,15-pentaoxaoctadecan-18-one

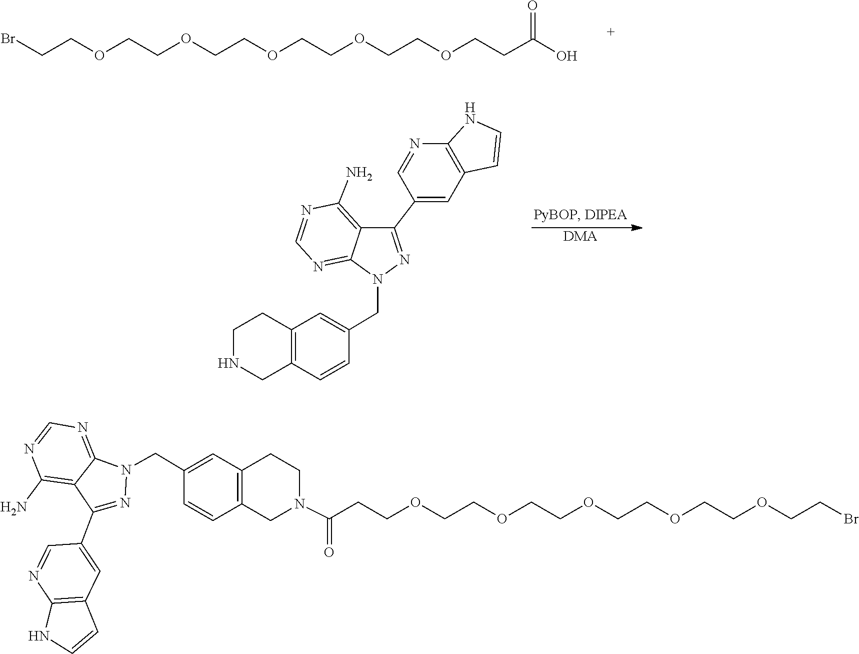

To a solution of 1-bromo-3,6,9,12,15-pentaoxaoctadecan-18-oic acid (105 mg, 282 μmol, 1.2 equiv) and 3-{1H-pyrrolo[2,3-b]pyridin-5-yl}-1-[(1,2,3,4-tetrahydroisoquinolin-6-yl)methyl]-1H-pyrazolo[3,4-d]pyrimidin-4-amine (120 mg, 235 μmol, 1.0 equiv) in DMA (2.34 mL) was added DIPEA (163 μL, 940 μmol, 4.0 equiv) followed by PyBOP (158 mg, 305 μmol, 1.3 equiv). The resulting solution was stirred at room temperature for 3 h then purified by reverse phase HPLC (10→98% MeCN+0.1% formic acid/H₂O+0.1% formic acid) to afford the product (82.7 mg, 47% yield). LCMS (ESI) m/z: [M+H] calcd for C₃₅H₄₃BrN₈O₆: 751.26; found 751.2.

Following General Procedure 5, but using the appropriate halide containing PEG carboxylic acid and amine containing active site inhibitors in Table 2, the Intermediates B3 in Table 10 were prepared:

TABLE 10
Additional PEG halides prepared

| | Molecular | Calculated | Observed |
|---|---|---|---|
| Structure | Formula | MW | MW |
| 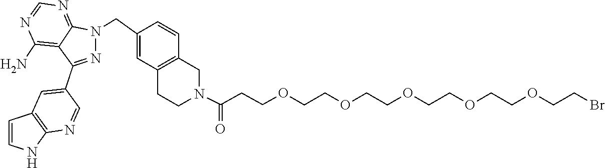 [SMILES: Nc1ncnc2c1c(-c1cnc3[nH]ccc3c1)nn2Cc1ccc2c(c1)CCN(C(=O)CCOCCOCCOCCOCCOCCBr)C2] | C₃₅H₄₃BrN₈O₆ | [M + H] = 751.26 | [M + H] = 751.2 |
| Intermediate B3-1 | | | |
| 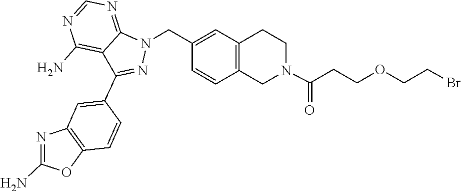 [SMILES: Nc1nc2cc(-c3nn(Cc4ccc5c(c4)CCN(C(=O)CCOCCBr)C5)c4ncnc(N)c34)ccc2o1] | C₂₇H₂₇BrN₈O₃ | [M + H] = 591.15 | [M + H] = 591.2 |
| Intermediate B3-2 | | | |

### General Procedure 6: Displacement of a PEG Halide with an Amine Containing Post Linker and Deprotection of the Amine

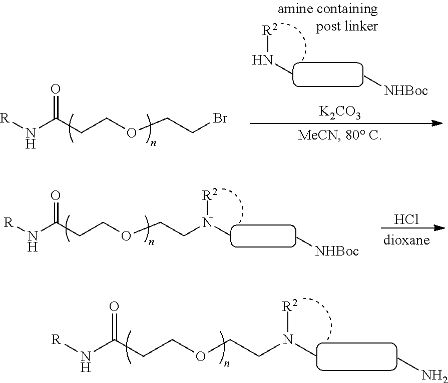

### Step 1

To a 0.1 M solution of halide containing PEG (1.0 equiv) in MeCN was added K₂CO₃ (3.0 equiv) followed by amine containing post linker (1.2 equiv). The resulting suspension was heated to 80° C. and stirred until consumption of the PEG halide, as indicated by LCMS analysis. The reaction was cooled to room temperature and then purified by silica gel chromatography to afford the product.

### Step 2

To a 0.07 M solution of N-Boc protected amine (1.0 equiv) in dioxane was added HCl (4 M in dioxane, 10.0 equiv). The reaction was stirred until consumption of N-Boc protected amine, as indicated by LCMS analysis. The reaction was then concentrated under reduced pressure to afford the product.

### Intermediate B2-4. 18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-1-(4-{5H,6H,7H,8H-pyrido[4,3-d]pyrimidin-2-yl}piperazin-1-yl)-3,6,9,12,15-pentaoxaoctadecan-18-one

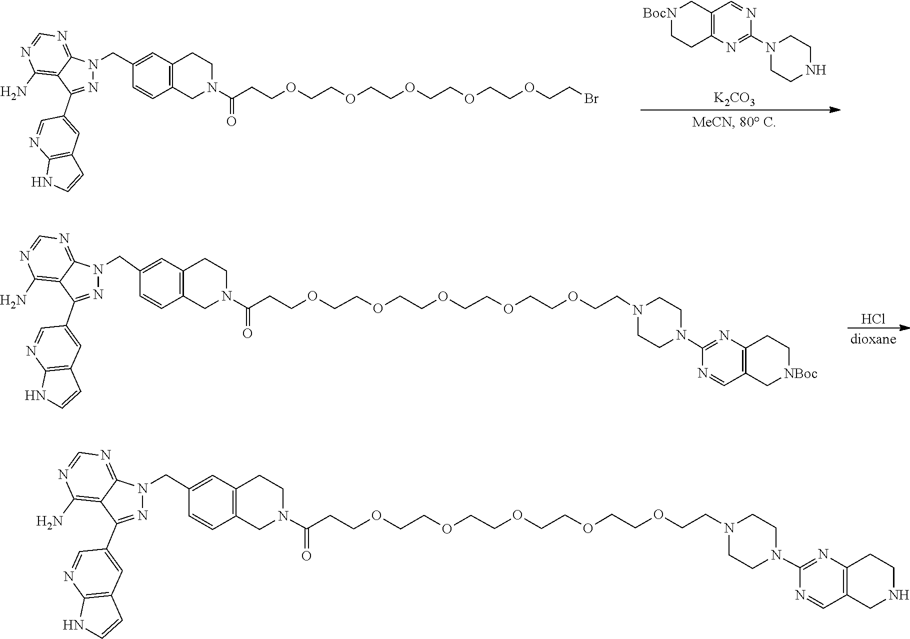 [SMILES: Nc1ncnc2c1c(-c1cnc3[nH]ccc3c1)nn2Cc1ccc2c(c1)CCN(C(=O)CCOCCOCCOCCOCCOCCBr)C2 ; "K2CO3" ; CC(C)(C)OC(=O)N1CCc2nc(N3CCNCC3)ncc2C1 ; CC(C)(C)OC(=O)N1CCc2nc(N3CCN(CCOCCOCCOCCOCCOCCC(=O)N4CCc5cc(Cn6nc(-c7cnc8[nH]ccc8c7)c7c(N)ncnc76)ccc5C4)CC3)ncc2C1 ; Cl ; Nc1ncnc2c1c(-c1cnc3[nH]ccc3c1)nn2Cc1ccc2c(c1)CCN(C(=O)CCOCCOCCOCCOCCOCCN1CCN(c3ncc4c(n3)CCNC4)CC1)C2]

### Step 1: Synthesis of tert-butyl 2-[4-(18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-18-oxo-3,6,9,12,15-pentaoxaoctadecan-1-yl)piperazin-1-yl]-5H,6H,7H,8H-pyrido[4,3-d]pyrimidine-6-carboxylate

To a suspension of 18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-1-bromo-3,6,9,12,15-pentaoxaoctadecan-18-one (82.7 mg, 110 μmol, 1.0 equiv) in MeCN (1.09 mL) was added K₂CO₃ (45.6 mg, 330 μmol, 3.0 equiv) followed by tert-butyl 2-(piperazin-1-yl)-5H,6H,7H,8H-pyrido[4,3-d]pyrimidine-6-carboxylate (42.1 mg, 132 μmol, 1.2 equiv). The resulting suspension was heated to 80° C. for 8 h, then purified by silica gel chromatography (0-20% MeOH/DCM) to afford the product (75.1 mg, 70% yield). LCMS (ESI) m/z: [M+H] calcd for C₅₁H₆₇N₁₃O₈: 990.53; found 990.5.

### Step 2: Synthesis of 18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-1-(4-{5H,6H,7H,8H-pyrido[4,3-d]pyrimidin-2-yl}piperazin-1-yl)-3,6,9,12,15-pentaoxaoctadecan-18-one

To a solution of tert-butyl 2-[4-(18-{6-[(4-amino-3-{1H-pyrrolo[2,3-b]pyridin-5-yl}-1H-pyrazolo[3,4-d]pyrimidin-1-yl)methyl]-1,2,3,4-tetrahydroisoquinolin-2-yl}-18-oxo-3,6,9,12,15-pentaoxaoctadecan-1-yl)piperazin-1-yl]-5H,6H,7H,8H-pyrido[4,3-d]pyrimidine-6-carboxylate (75.1 mg, 75.8 μmol, 1.0 equiv) in dioxane (1 mL) was added HCl (4 M in dioxane, 472 μL, 1.89 mmol, 10.0 equiv). The solution was stirred at room temperature for 45 min, then concentrated under reduced pressure to afford the product. LCMS (ESI) m/z: [M+Na] calcd for C₄₆H₅₉N₁₃O₆: 912.46; found 912.5.

Following General Procedure 6, but using the appropriate PEG carboxylic acid and amine containing active site inhibitors in Table 2, the Intermediates B2 in Table 11 were prepared:

TABLE 11
Additional amines prepared

| | Molecular | Calculated | Observed |
|---|---|---|---|
| Structure | Formula | MW | MW |
| 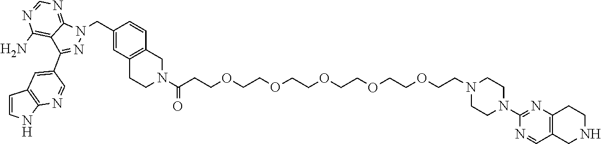 [SMILES: Nc1ncnc2c1c(-c1cnc3[nH]ccc3c1)nn2Cc1ccc2c(c1)CCN(C(=O)CCOCCOCCOCCOCCOCCN1CCN(c3ncc4c(n3)CCNC4)CC1)C2] | C₄₆H₅₉N₁₃O₆ | [M + H] = 890.48 | [M + H] = 890.5 |
| Intermediate B2-4 | | | |

Following General Procedure 1, but using the appropriate carboxylic acid PEG tert-butyl ester and amine containing active site inhibitors in Table 2, the Intermediates B4 in Table 12 were prepared:

TABLE 12
Additional amines prepared

| | Molecular | Calculated | Observed |
|---|---|---|---|
| Structure | Formula | MW | MW |
| 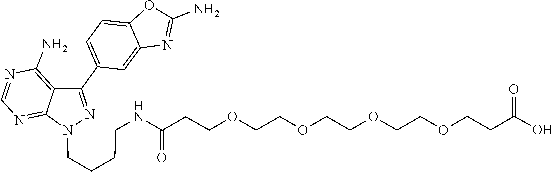 [SMILES: Nc1nc2cc(-c3nn(CCCCNC(=O)CCOCCOCCOCCOCCC(=O)O)c4ncnc(N)c34)ccc2o1] | C₂₈H₃₈N₈O₈ | [M + H] = 615.29 | [M + H] = 615.1 |
| Intermediate B4-1 | | | |

Following General Procedure 1, but using the appropriate Intermediates B4 in Table 12 and amine containing pre-linkers in Table 4, the Intermediates B2 in Table 13 were prepared:

TABLE 13
Additional amines prepared

| | Molecular | Calculated | Observed |
|---|---|---|---|
| Structure | Formula | MW | MW |
| 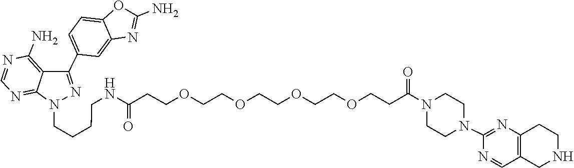 [SMILES: Nc1nc2cc(-c3nn(CCCCNC(=O)CCOCCOCCOCCOCCC(=O)N4CCN(c5ncc6c(n5)CCNC6)CC4)c4ncnc(N)c34)ccc2o1] | C₃₉H₅₃N₁₃O₇ | [M + H] = 816.43 | [M + H] = 816.4 |
| Intermediate B2-5 | | | |

Following General Procedure 1, but using the appropriate Intermediates A1 and amine containing pre-linkers in Table 4, the Intermediates B2 in Table 14 were prepared:

TABLE 14
Additional amines prepared

| | Molecular | Calculated | Observed |
|---|---|---|---|
| Structure | Formula | MW | MW |
| 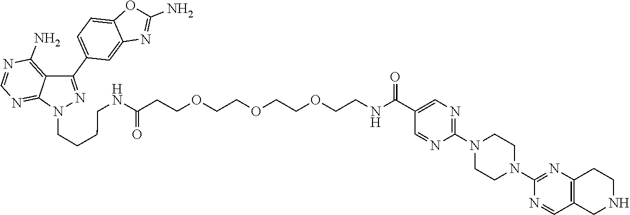 [SMILES: Nc1nc2cc(-c3nn(CCCCNC(=O)CCOCCOCCOCCNC(=O)c4cnc(N5CCN(c6ncc7c(n6)CCNC7)CC5)nc4)c4ncnc(N)c34)ccc2o1] | C₄₁H₅₂N₁₆O₆ | [M + H] = 865.44 | [M + H] = 865.2 |
| Intermediate B2-6 | | | |
| 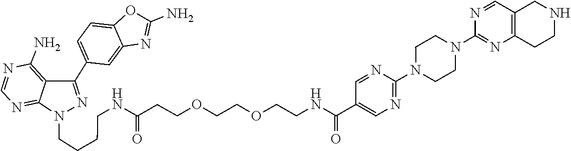 [SMILES: Nc1nc2cc(-c3nn(CCCCNC(=O)CCOCCOCCNC(=O)c4cnc(N5CCN(c6ncc7c(n6)CCNC7)CC5)nc4)c4ncnc(N)c34)ccc2o1] | C₃₉H₄₈N₁₆O₅ | [M + H] = 821.41 | [M + H] = 821.2 |
| Intermediate B2-7 | | | |
| 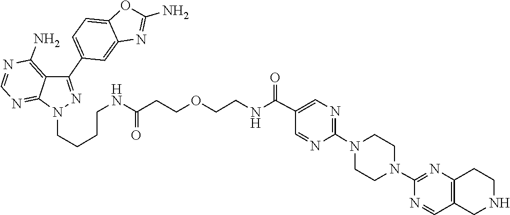 [SMILES: Nc1nc2cc(-c3nn(CCCCNC(=O)CCOCCNC(=O)c4cnc(N5CCN(c6ncc7c(n6)CCNC7)CC5)nc4)c4ncnc(N)c34)ccc2o1] | C₃₇H₄₄N₁₆O₄ | [M + H] = 777.38 | [M + H] = 777.3 |
| Intermediate B2-8 | | | |
| 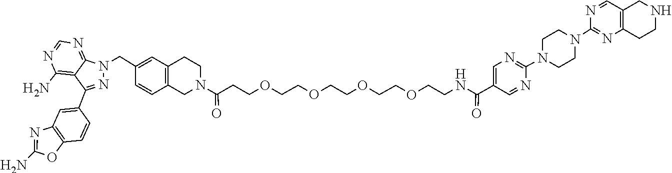 [SMILES: Nc1nc2cc(-c3nn(Cc4ccc5c(c4)CCN(C(=O)CCOCCOCCOCCOCCNC(=O)c4cnc(N6CCN(c7ncc8c(n7)CCNC8)CC6)nc4)C5)c4ncnc(N)c34)ccc2o1] | C₄₉H₅₈N₁₆O₇ | [M + H] = 983.48 | [M + H] = 983.4 |
| Intermediate B2-9 | | | |
| 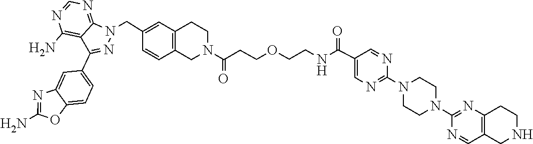 [SMILES: Nc1nc2cc(-c3nn(Cc4ccc5c(c4)CCN(C(=O)CCOCCNC(=O)c4cnc(N6CCN(c7ncc8c(n7)CCNC8)CC6)nc4)C5)c4ncnc(N)c34)ccc2o1] | C₄₃H₄₆N₁₆O₄ | [M + H] = 851.40 | [M + H] = 851.4 |
| Intermediate B2-10 | | | |
| 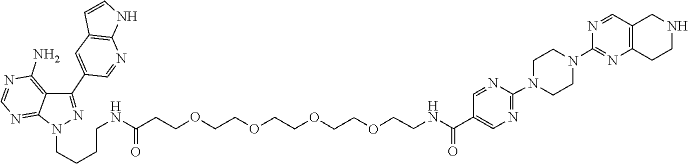 [SMILES: Nc1ncnc2c1c(-c1cnc3[nH]ccc3c1)nn2CCCCNC(=O)CCOCCOCCOCCOCCNC(=O)c1cnc(N2CCN(c3ncc4c(n3)CCNC4)CC2)nc1] | C₄₃H₅₆N₁₆O₆ | [M + H] = 893.47 | [M + H] = 893.3 |
| Intermediate B2-11 | | | |
| 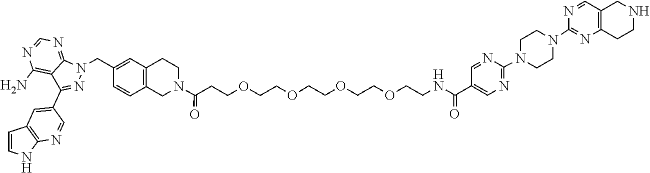 [SMILES: Nc1ncnc2c1c(-c1cnc3[nH]ccc3c1)nn2Cc1ccc2c(c1)CCN(C(=O)CCOCCOCCOCCOCCNC(=O)c1cnc(N3CCN(c4ncc5c(n4)CCNC5)CC3)nc1)C2] | C₄₉H₅₈N₁₆O₆ | [M + Na] = 989.46 | [M + Na] = 989.4 |
| Intermediate B2-12 | | | |
| 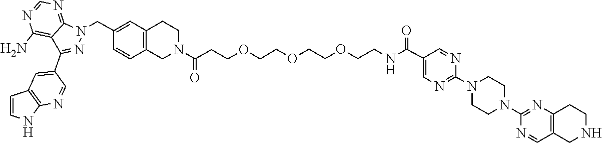 [SMILES: Nc1ncnc2c1c(-c1cnc3[nH]ccc3c1)nn2Cc1ccc2c(c1)CCN(C(=O)CCOCCOCCOCCNC(=O)c1cnc(N3CCN(c4ncc5c(n4)CCNC5)CC3)nc1)C2] | C₄₇H₅₄N₁₆O₅ | [M + H] = 923.46 | [M + H] = 923.4 |
| Intermediate B2-13 | | | |
| 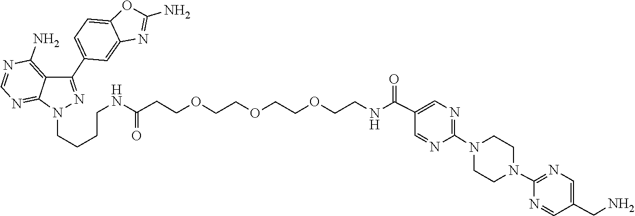 [SMILES: NCc1cnc(N2CCN(c3ncc(C(=O)NCCOCCOCCOCCC(=O)NCCCCn4nc(-c5ccc6oc(N)nc6c5)c5c(N)ncnc54)cn3)CC2)nc1] | C₃₉H₅₀N₁₆O₆ | [M + H] = 839.42 | [M + H] = 893.3 |
| Intermediate B2-14 | | | |
| 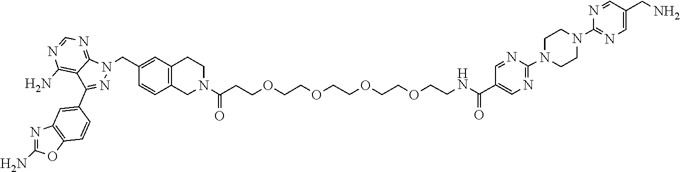 [SMILES: NCc1cnc(N2CCN(c3ncc(C(=O)NCCOCCOCCOCCOCCC(=O)N4CCc5cc(Cn6nc(-c7ccc8oc(N)nc8c7)c7c(N)ncnc76)ccc5C4)cn3)CC2)nc1] | C₄₇H₅₆N₁₆O₇ | [M + H] = 957.46 | [M + H] = 957.7 |
| Intermediate B2-15 | | | |
| 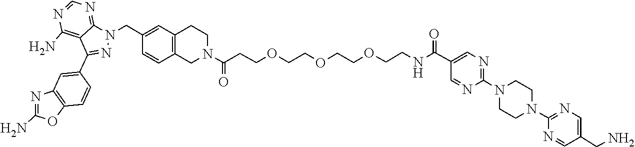 [SMILES: NCc1cnc(N2CCN(c3ncc(C(=O)NCCOCCOCCOCCC(=O)N4CCc5cc(Cn6nc(-c7ccc8oc(N)nc8c7)c7c(N)ncnc76)ccc5C4)cn3)CC2)nc1] | C₄₅H₅₂N₁₆O₆ | [M + Na] = 935.42 | [M + Na] = 935.3 |
| Intermediate B2-16 | | | |
| 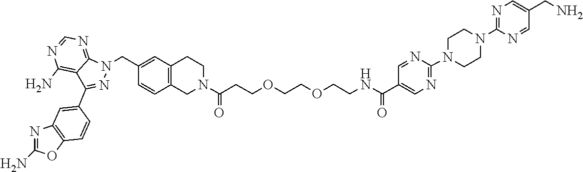 [SMILES: NCc1cnc(N2CCN(c3ncc(C(=O)NCCOCCOCCC(=O)N4CCc5cc(Cn6nc(-c7ccc8oc(N)nc8c7)c7c(N)ncnc76)ccc5C4)cn3)CC2)nc1] | C₄₃H₄₈N₁₆O₅ | [M + Na] = 891.39 | [M + Na] = 891.4 |
| Intermediate B2-17 | | | |
| 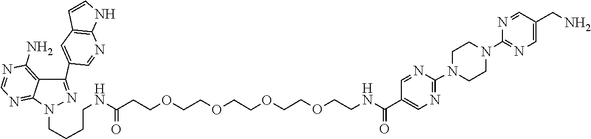 [SMILES: NCc1cnc(N2CCN(c3ncc(C(=O)NCCOCCOCCOCCOCCC(=O)NCCCCn4nc(-c5cnc6[nH]ccc6c5)c5c(N)ncnc54)cn3)CC2)nc1] | C₄₁H₅₄N₁₆O₆ | [M + H] = 867.45 | [M + H] = 867.3 |
| Intermediate B2-18 | | | |
| 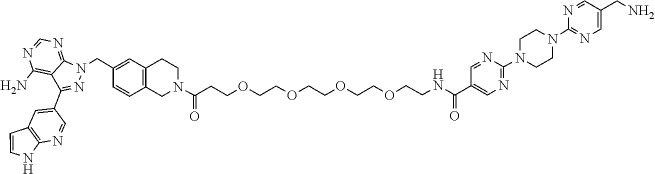 [SMILES: NCc1cnc(N2CCN(c3ncc(C(=O)NCCOCCOCCOCCOCCC(=O)N4CCc5cc(Cn6nc(-c7cnc8[nH]ccc8c7)c7c(N)ncnc76)ccc5C4)cn3)CC2)nc1] | C₄₇H₅₆N₁₆O₆ | [M + H] = 941.47 | [M + H] = 941.2 |
| Intermediate B2-19 | | | |
| 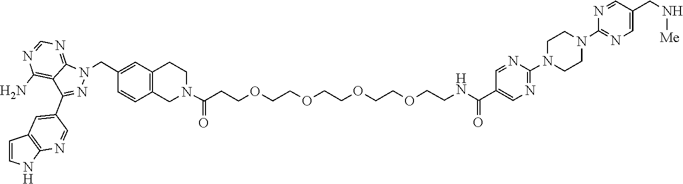 [SMILES: CNCc1cnc(N2CCN(c3ncc(C(=O)NCCOCCOCCOCCOCCC(=O)N4CCc5cc(Cn6nc(-c7cnc8[nH]ccc8c7)c7c(N)ncnc76)ccc5C4)cn3)CC2)nc1] | C₄₈H₅₈N₁₆O₆ | [M + H] = 955.48 | [M + H] = 955.2 |
| Intermediate B2-20 | | | |
| 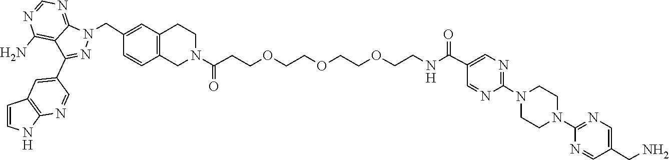 [SMILES: NCc1cnc(N2CCN(c3ncc(C(=O)NCCOCCOCCOCCC(=O)N4CCc5cc(Cn6nc(-c7cnc8[nH]ccc8c7)c7c(N)ncnc76)ccc5C4)cn3)CC2)nc1] | C₄₅H₅₂N₁₆O₅ | [M + H] = 897.44 | [M + H] = 897.3 |
| Intermediate B2-21 | | | |
| 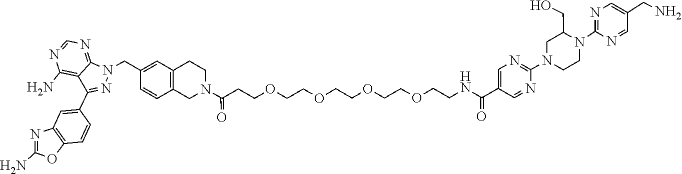 [SMILES: NCc1cnc(N2CCN(c3ncc(C(=O)NCCOCCOCCOCCOCCC(=O)N4CCc5cc(Cn6nc(-c7ccc8oc(N)nc8c7)c7c(N)ncnc76)ccc5C4)cn3)CC2CO)nc1] | C₄₈H₅₈N₁₆O₈ | [M + H] = 987.47 | [M + H] = 987.42 |
| Intermediate B2-22 | | | |
| 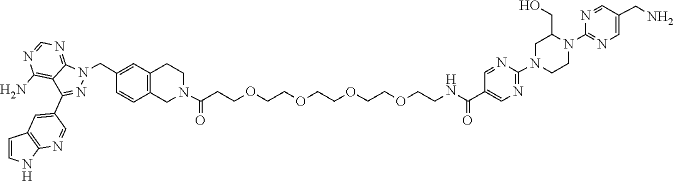 [SMILES: NCc1cnc(N2CCN(c3ncc(C(=O)NCCOCCOCCOCCOCCC(=O)N4CCc5cc(Cn6nc(-c7cnc8[nH]ccc8c7)c7c(N)ncnc76)ccc5C4)cn3)CC2CO)nc1] | C₄₈H₅₈N₁₆O₇ | [M + H] = 971.48 | [M + H] = 971.31 |
| Intermediate B2-23 | | | |
| 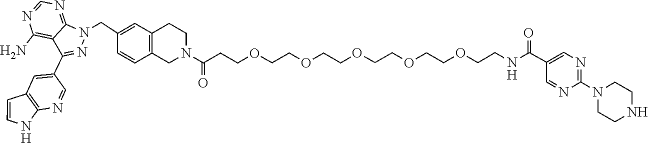 [SMILES: Nc1ncnc2c1c(-c1cnc3[nH]ccc3c1)nn2Cc1ccc2c(c1)CCN(C(=O)CCOCCOCCOCCOCCOCCNC(=O)c1cnc(N3CCNCC3)nc1)C2] | C₄₄H₅₅N₁₃O₇ | [M + H] = 878.44 | [M + H] = 878.5 |
| Intermediate B2-24 | | | |
| 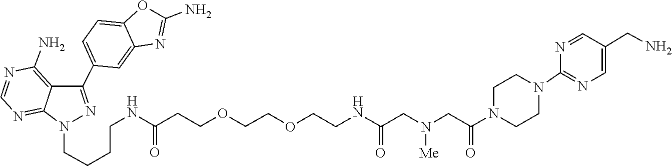 [SMILES: CN(CC(=O)NCCOCCOCCC(=O)NCCCCn1nc(-c2ccc3oc(N)nc3c2)c2c(N)ncnc21)CC(=O)N1CCN(c2ncc(CN)cn2)CC1] | C₃₇H₅₁N₁₅O₆ | [M + H] = 802.42 | [M + H] = 802.4 |
| Intermediate B2-25 | | | |
| 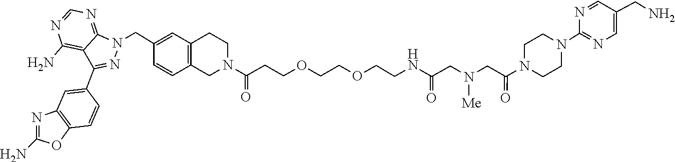 [SMILES: CN(CC(=O)NCCOCCOCCC(=O)N1CCc2cc(Cn3nc(-c4ccc5oc(N)nc5c4)c4c(N)ncnc43)ccc2C1)CC(=O)N1CCN(c2ncc(CN)cn2)CC1] | C₄₃H₅₃N₁₅O₆ | [M + H] = 876.44 | [M + H] = 876.4 |
| Intermediate B2-26 | | | |
| 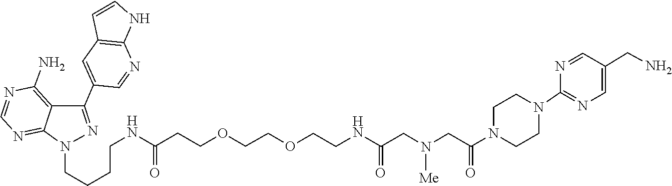 [SMILES: CN(CC(=O)NCCOCCOCCC(=O)NCCCCn1nc(-c2cnc3[nH]ccc3c2)c2c(N)ncnc21)CC(=O)N1CCN(c2ncc(CN)cn2)CC1] | C₃₇H₅₁N₁₅O₅ | [M + H] = 786.43 | [M + H] = 786.5 |
| Intermediate B2-27 | | | |
| 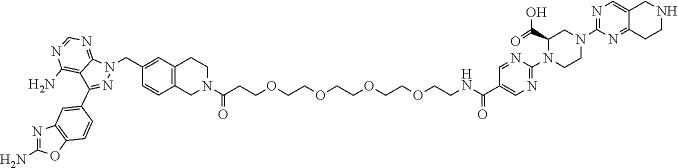 [SMILES: Nc1nc2cc(-c3nn(Cc4ccc5c(c4)CCN(C(=O)CCOCCOCCOCCOCCNC(=O)c4cnc(N6CCN(c7ncc8c(n7)CCNC8)C[C@@H]6C(=O)O)nc4)C5)c4ncnc(N)c34)ccc2o1] | C₅₀H₅₈N₁₆O₉ | [M + H] = 1027.47 | [M + H] = 1027.1 |
| Intermediate B2-28 | | | |
| 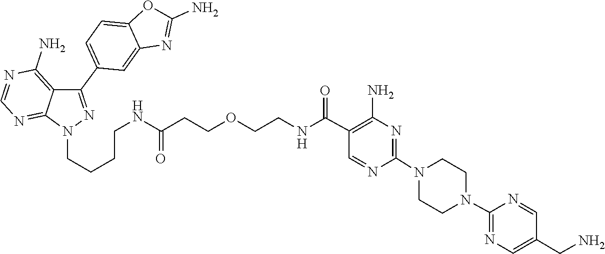 [SMILES: NCc1cnc(N2CCN(c3ncc(C(=O)NCCOCCC(=O)NCCCCn4nc(-c5ccc6oc(N)nc6c5)c5c(N)ncnc54)c(N)n3)CC2)nc1] | C₃₅H₄₃N₁₇O₄ | [M + H] = 766.38 | [M + H] = 766.3 |
| Intermediate B2-29 | | | |
| 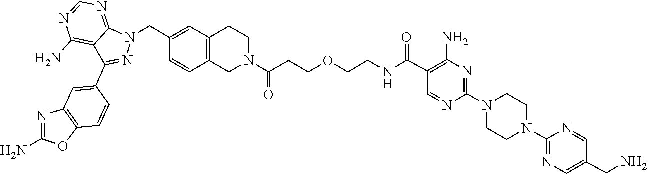 [SMILES: NCc1cnc(N2CCN(c3ncc(C(=O)NCCOCCC(=O)N4CCc5cc(Cn6nc(-c7ccc8oc(N)nc8c7)c7c(N)ncnc76)ccc5C4)c(N)n3)CC2)nc1] | C₄₁H₄₅N₁₇O₄ | [M + H] = 840.39 | [M + H] = 840.4 |
| Intermediate B2-30 | | | |
| 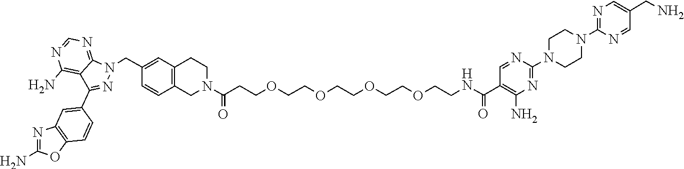 [SMILES: NCc1cnc(N2CCN(c3ncc(C(=O)NCCOCCOCCOCCOCCC(=O)N4CCc5cc(Cn6nc(-c7ccc8oc(N)nc8c7)c7c(N)ncnc76)ccc5C4)c(N)n3)CC2)nc1] | C₄₇H₅₇N₁₇O₇ | [M + H] = 972.47 | [M + H] = 972.5 |
| Intermediate B2-31 | | | |
| 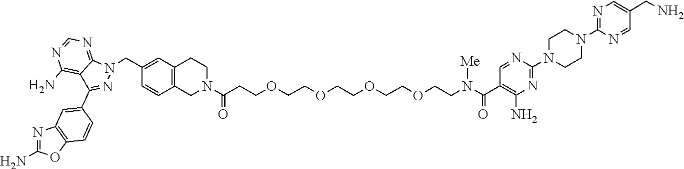 [SMILES: CN(CCOCCOCCOCCOCCC(=O)N1CCc2cc(Cn3nc(-c4ccc5oc(N)nc5c4)c4c(N)ncnc43)ccc2C1)C(=O)c1cnc(N2CCN(c3ncc(CN)cn3)CC2)nc1N] | C₄₈H₅₉N₁₇O₇ | [M + H] = 986.49 | [M + H] = 986.4 |
| Intermediate B2-32 | | | |
| 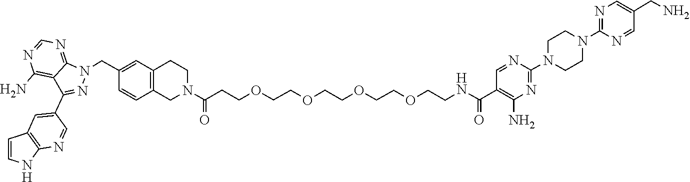 [SMILES: NCc1cnc(N2CCN(c3ncc(C(=O)NCCOCCOCCOCCOCCC(=O)N4CCc5cc(Cn6nc(-c7cnc8[nH]ccc8c7)c7c(N)ncnc76)ccc5C4)c(N)n3)CC2)nc1] | C₄₇H₅₇N₁₇O₆ | [M + H] = 956.48 | [M + H] = 956.3 |
| Intermediate B2-33 | | | |
| 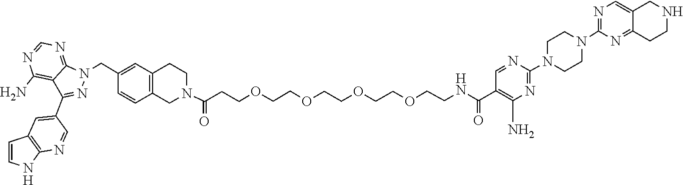 [SMILES: Nc1nc(N2CCN(c3ncc4c(n3)CCNC4)CC2)ncc1C(=O)NCCOCCOCCOCCOCCC(=O)N1CCc2cc(Cn3nc(-c4cnc5[nH]ccc5c4)c4c(N)ncnc43)ccc2C1] | C₄₉H₅₉N₁₇O₆ | [M + H] = 982.49 | [M + H] = 982.2 |
| Intermediate B2-34 | | | |
| 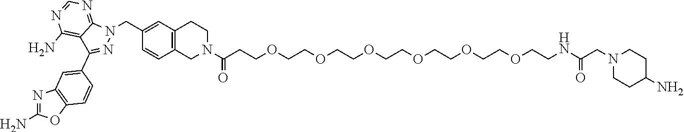 [SMILES: Nc1nc2cc(-c3nn(Cc4ccc5c(c4)CCN(C(=O)CCOCCOCCOCCOCCOCCOCCNC(=O)CN4CCC(N)CC4)C5)c4ncnc(N)c34)ccc2o1] | C₄₄H₆₁N₁₁O₉ | [M + H] = 888.48 | [M + H] = 888.3 |
| Intermediate B2-35 | | | |
| 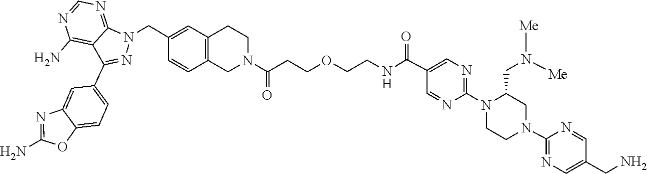 [SMILES: CN(C)C[C@@H]1CN(c2ncc(CN)cn2)CCN1c1ncc(C(=O)NCCOCCC(=O)N2CCc3cc(Cn4nc(-c5ccc6oc(N)nc6c5)c5c(N)ncnc54)ccc3C2)cn1] | C₄₄H₅₁N₁₇O₄ | [M + H] = 882.44 | [M + H] = 882.4 |
| Intermediate B2-36 | | | |
| 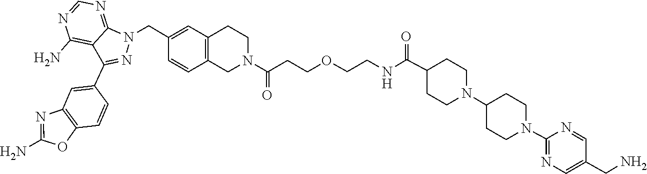 [SMILES: NCc1cnc(N2CCC(N3CCC(C(=O)NCCOCCC(=O)N4CCc5cc(Cn6nc(-c7ccc8oc(N)nc8c7)c7c(N)ncnc76)ccc5C4)CC3)CC2)nc1] | C₄₃H₅₂N₁₄O₄ | [M + H] = 829.44 | [M + H] = 829.3 |
| Intermediate B2-37 | | | |
| 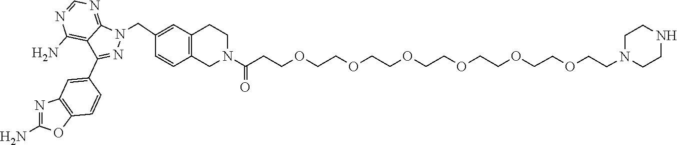 [SMILES: Nc1nc2cc(-c3nn(Cc4ccc5c(c4)CCN(C(=O)CCOCCOCCOCCOCCOCCOCCN4CCNCC4)C5)c4ncnc(N)c34)ccc2o1] | C₄₁H₅₆N₁₀O₈ | [M + H] = 817.44 | [M + H] = 817.2 |
| Intermediate B2-38 | | | |
| 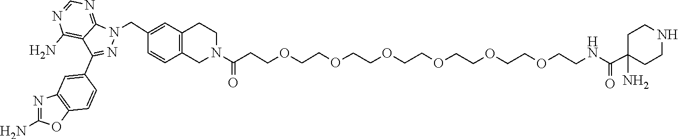 [SMILES: Nc1nc2cc(-c3nn(Cc4ccc5c(c4)CCN(C(=O)CCOCCOCCOCCOCCOCCOCCNC(=O)C4(N)CCNCC4)C5)c4ncnc(N)c34)ccc2o1] | C₄₃H₅₉N₁₁O₉ | [M + H] = 874.46 | [M + H] = 874.3 |
| Intermediate B2-39 | | | |

Following General Procedure 2, but using the appropriate 4-nitrophenyl carbonate containing rapamycin monomer in Table 1 and Intermediates B2 from Tables 9, 11, and 13 and 14, the Series 2 bivalent analogs in Table 15 were synthesized:

TABLE 15
Series 2 Bivalent Compounds:

| | Molecular | Calculated | Observed |
|---|---|---|---|
| Structure | Formula | MW | MW |
| 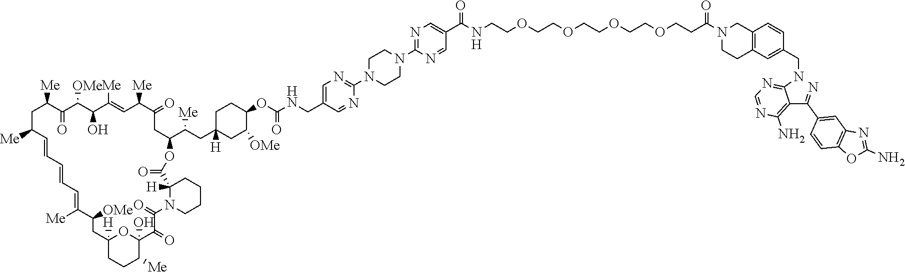 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₃₃N₁₇O₂₁ | [M + H] = 1896.99 | [M + H] = 1896.7 |
| Example 27 | | | |
| 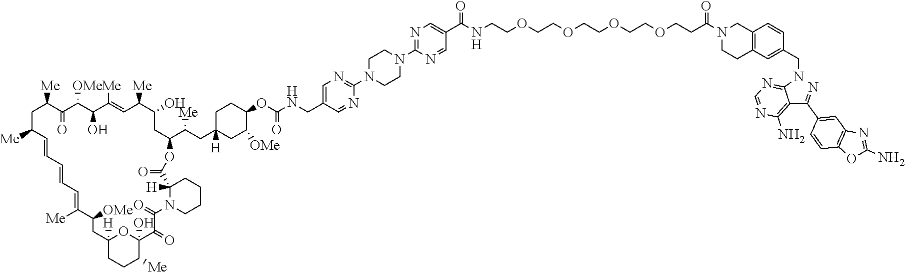 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₉H₁₃₅N₁₇O₂₁ | [M + H] = 1899.01 | [M + H] = 1899.1 |
| Example 28 | | | |
| 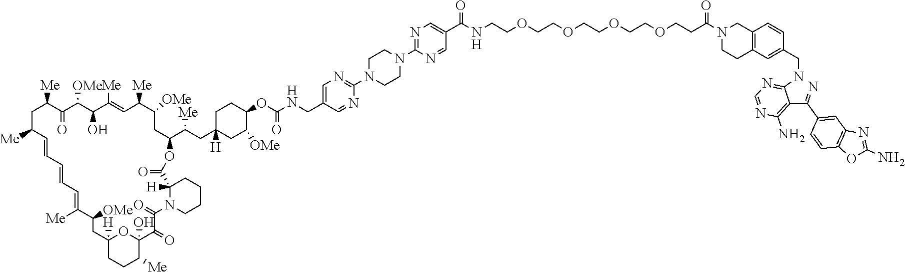 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₀H₁₃₇N₁₇O₂₁ | [M + H] = 1913.03 | [M + H] = 1913.0 |
| Example 29 | | | |
| 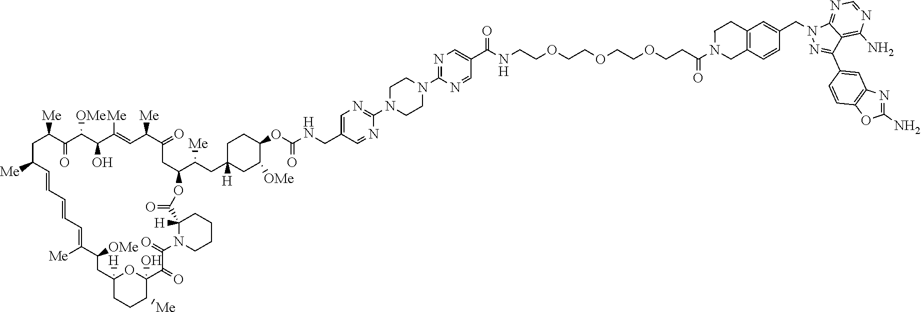 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₂₉N₁₇O₂₀ | [M + H] = 1852.97 | [M + H] = 1852.9 |
| Example 30 | | | |
| 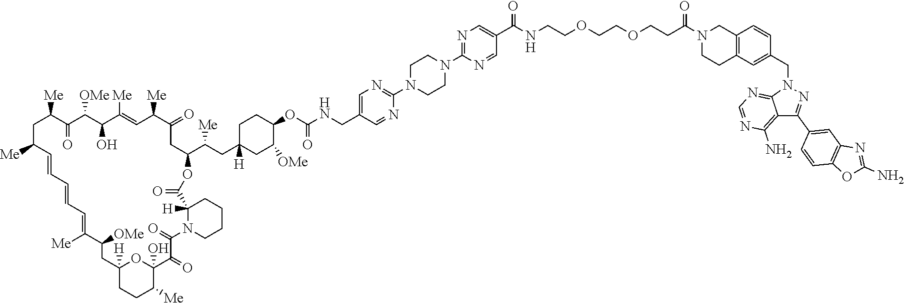 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCC(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₂₅N₁₇O₁₉ | [M + H] = 1808.94 | [M + H] = 1809.0 |
| Example 31 | | | |
| 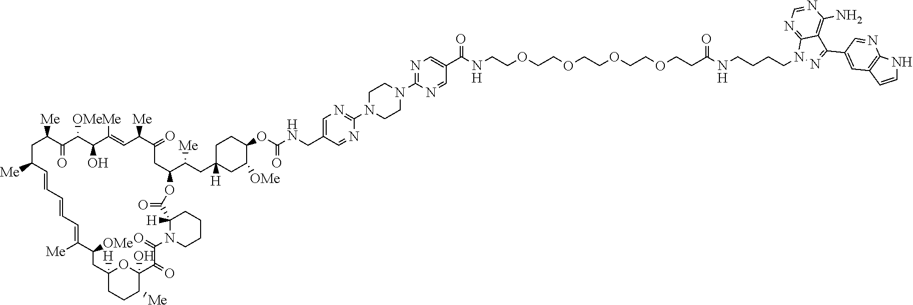 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₃₁N₁₇O₂₀ | [M + H] = 1806.98 | [M + H] = 1806.7 |
| Example 32 | | | |
| 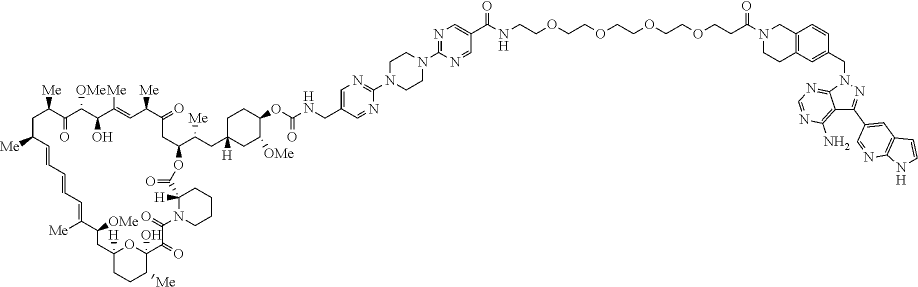 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₃N₁₇O₂₀ | [M + H] = 1881.00 | [M + H] = 1881.0 |
| Example 33 | | | |
| 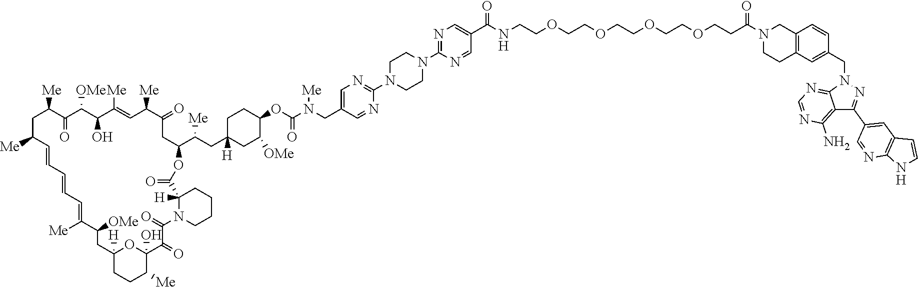 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N(C)Cc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₃₅N₁₇O₂₀ | [M + H] = 1895.01 | [M + H] = 1895.1 |
| Example 34 | | | |
| 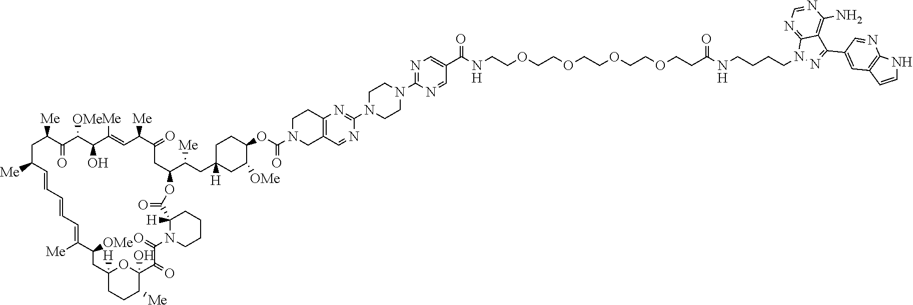 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)NCCCCn7nc(-c8cnc9[nH]ccc9c8)c8c(N)ncnc87)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₃N₁₇O₂₀ | [M + H] = 1833.00 | [M + H] = 1832.9 |
| Example 35 | | | |
| 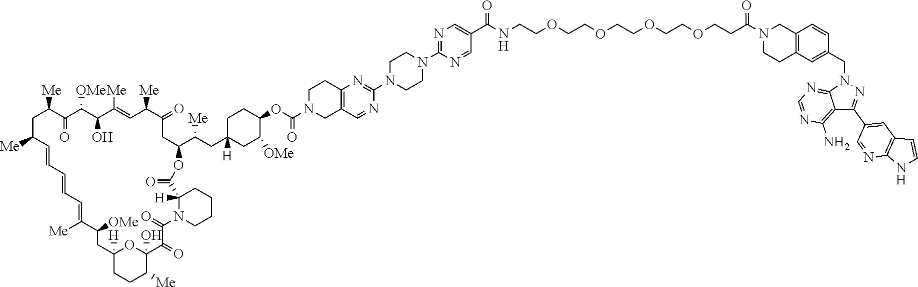 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₁H₁₃₅N₁₇O₂₀ | [M + H] = 1907.01 | [M + H] = 1907.0 |
| Example 36 | | | |
| 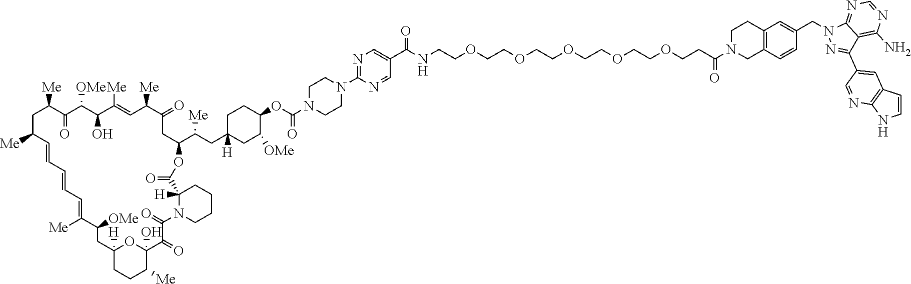 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(c4ncc(C(=O)NCCOCCOCCOCCOCCOCCC(=O)N5CCc6cc(Cn7nc(-c8cnc9[nH]ccc9c8)c8c(N)ncnc87)ccc6C5)cn4)CC3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₂N₁₄O₂₁ | [M + H] = 1817.98 | [M + H] = 1817.9 |
| Example 37 | | | |
| 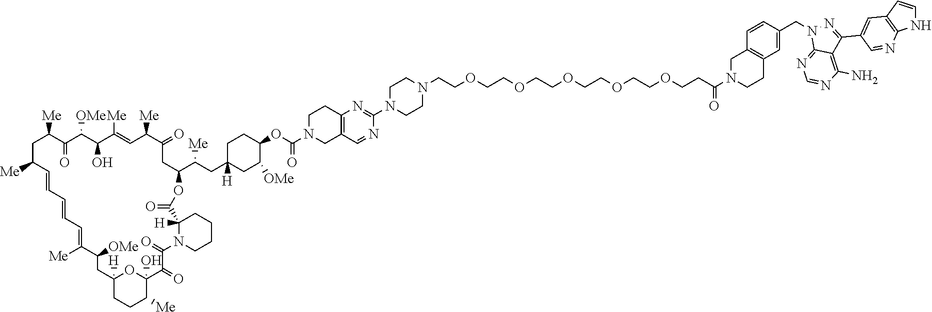 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCOCCOCCOCCOCCC(=O)N6CCc7cc(Cn8nc(-c9cnc%10[nH]ccc%10c9)c9c(N)ncnc98)ccc7C6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₈H₁₃₆N₁₄O₂₀ | [M + H] = 1830.01 | [M + H] = 1830.1 |
| Example 38 | | | |
| 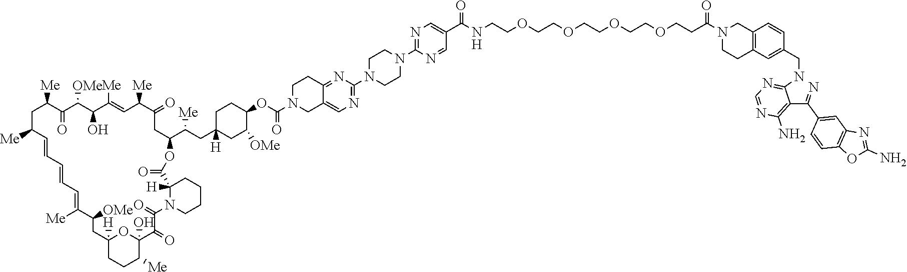 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₁H₁₃₅N₁₇O₂₁ | [M + H] = 1923.01 | [M + H] = 1923.1 |
| Example 39 | | | |
| 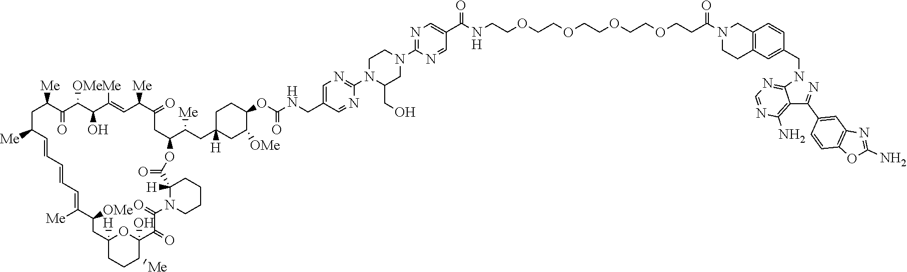 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₃₅N₁₇O₂₂ | [M + H] = 1927.01 | [M + H] = 1927.0 |
| Example 40 | | | |
| 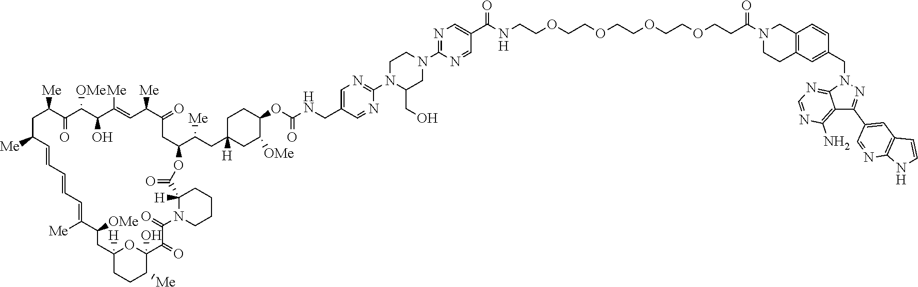 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)cn5)CC4CO)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₃₅N₁₇O₂₁ | [M + H] = 1911.01 | [M + H] = 1911.0 |
| Example 41 | | | |
| 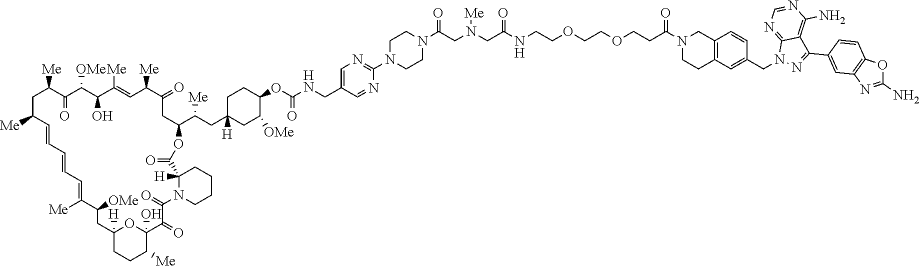 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CN(C)CC(=O)NCCOCCOCCC(=O)N5CCc6cc(Cn7nc(-c8ccc9oc(N)nc9c8)c8c(N)ncnc87)ccc6C5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₀N₁₆O₂₀ | [M + H] = 1815.97 | [M + H] = 1816.0 |
| Example 42 | | | |
| 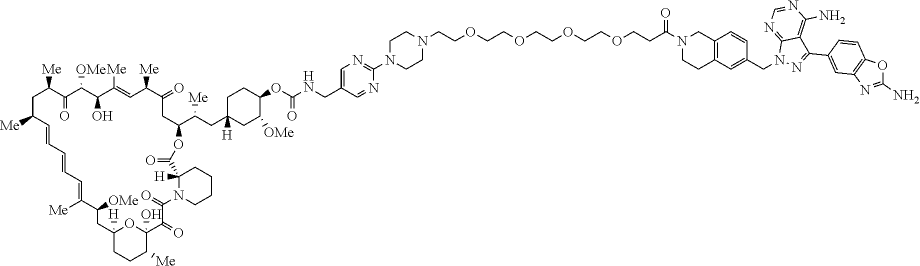 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)N5CCc6cc(Cn7nc(-c8ccc9oc(N)nc9c8)c8c(N)ncnc87)ccc6C5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₃₀N₁₄O₂₀ | [M + H] = 1775.97 | [M + H] = 1775.9 |
| Example 43 | | | |
| 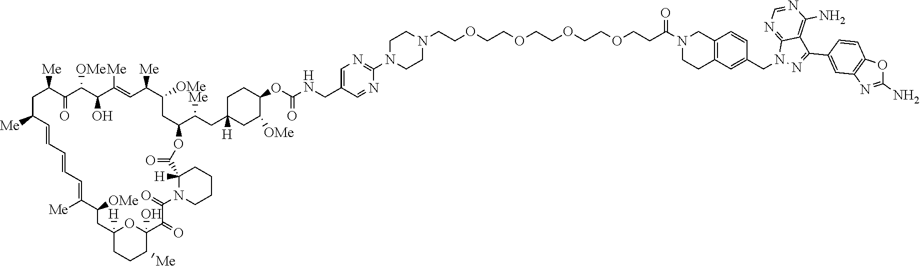 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)N5CCc6cc(Cn7nc(-c8ccc9oc(N)nc9c8)c8c(N)ncnc87)ccc6C5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₅H₁₃₄N₁₄O₂₀ | [M + H] = 1791.99 | [M + H] = 1791.8 |
| Example 44 | | | |
| 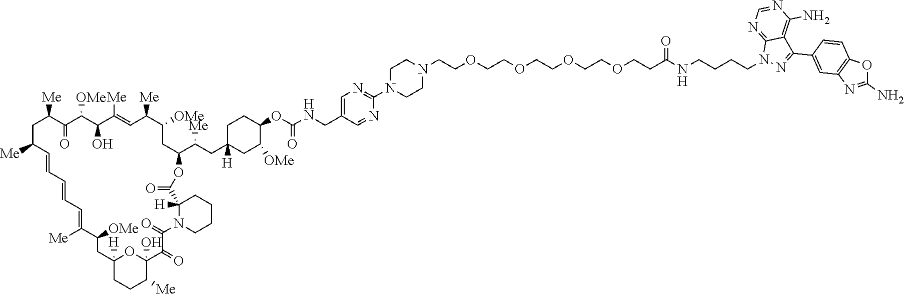 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCOCCOCCOCCC(=O)NCCCCn5nc(-c6ccc7oc(N)nc7c6)c6c(N)ncnc65)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₈₉H₁₃₂N₁₄O₂₀ | [M + H] = 1717.98 | [M + H] = 1717.9 |
| Example 45 | | | |
| 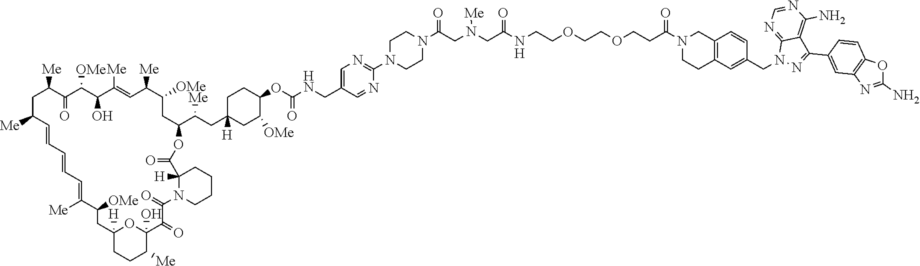 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CN(C)CC(=O)NCCOCCOCCC(=O)N5CCc6cc(Cn7nc(-c8ccc9oc(N)nc9c8)c8c(N)ncnc87)ccc6C5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₆H₁₃₄N₁₆O₂₀ | [M + H] = 1832.00 | [M + H] = 1832.0 |
| Example 103 | | | |
| 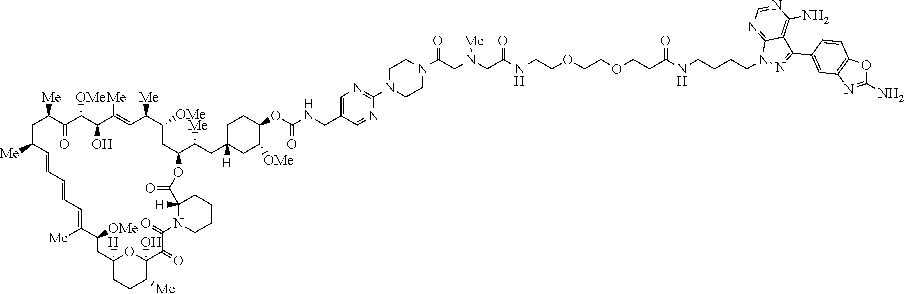 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CN(C)CC(=O)NCCOCCOCCC(=O)NCCCCn5nc(-c6ccc7oc(N)nc7c6)c6c(N)ncnc65)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₀H₁₃₂N₁₆O₂₀ | [M + H] = 1757.99 | [M + H] = 1757.9 |
| Example 104 | | | |
| 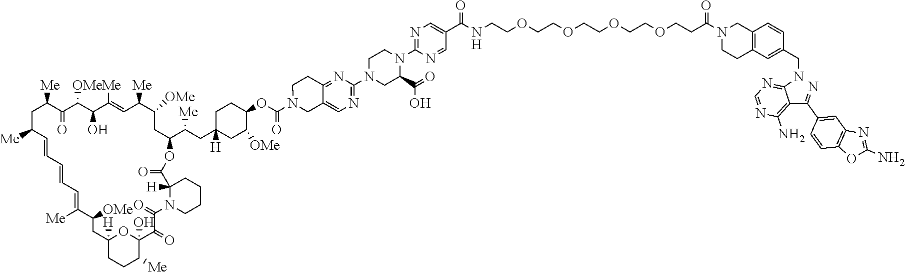 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)[C@@H](C(=O)O)C5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₃H₁₃₉N₁₇O₂₃ | [M + H] = 1983.03 | [M + H] = 1983.0 |
| Example 105 | | | |
| 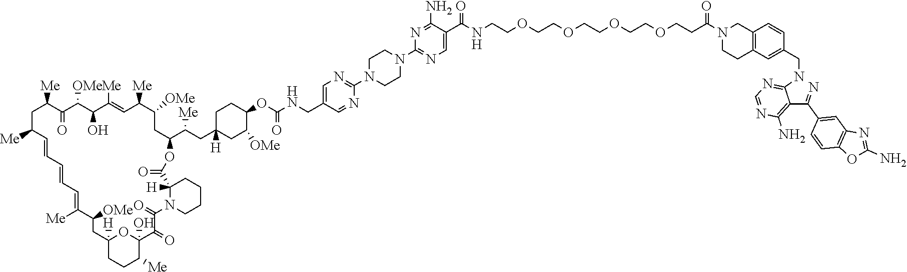 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9ccc%10oc(N)nc%10c9)c9c(N)ncnc98)ccc7C6)c(N)n5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₀H₁₃₈N₁₈O₂₁ | [M + H] = 1928.04 | [M + H] = 1927.9 |
| Example 106 | | | |
| 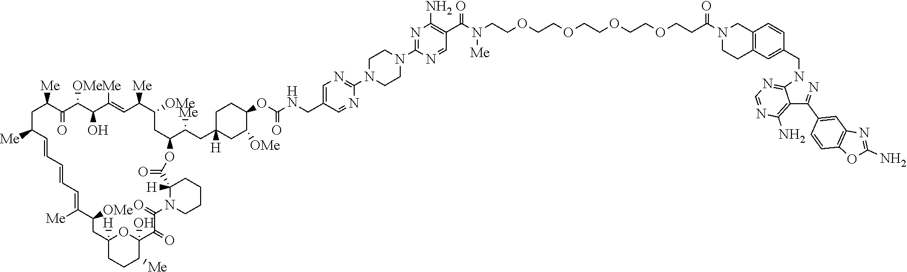 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N(C)CCOCCOCCOCCOCCC(=O)N6CCc7cc(Cn8nc(-c9ccc%10oc(N)nc%10c9)c9c(N)ncnc98)ccc7C6)c(N)n5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₁₀₁H₁₄₀N₁₈O₂₁ | [M + H] = 1942.05 | [M + H] = 1942.1 |
| Example 107 | | | |
| 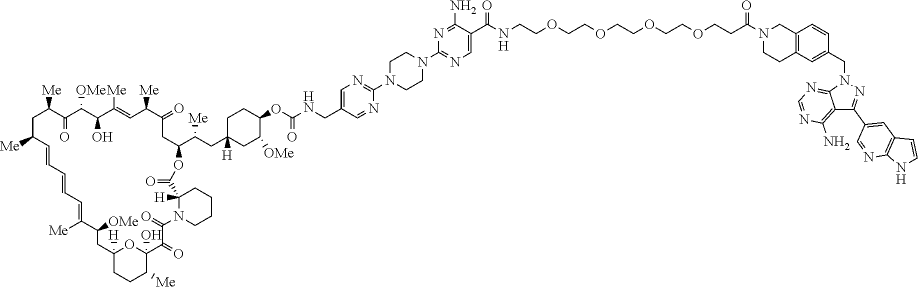 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCOCCOCCOCCC(=O)N6CCc7cc(Cn8nc(-c9cnc%10[nH]ccc%10c9)c9c(N)ncnc98)ccc7C6)c(N)n5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₄N₁₈O₂₀ | [M + H] = 1896.01 | [M + H] = 1896.0 |
| Example 108 | | | |
| 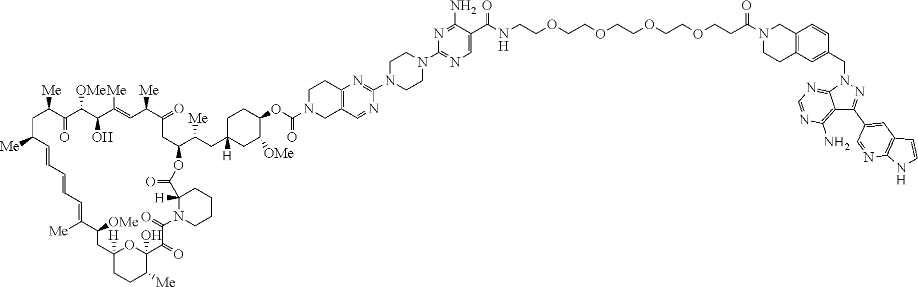 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)NCCOCCOCCOCCOCCC(=O)N7CCc8cc(Cn9nc(-c%10cnc%11[nH]ccc%11c%10)c%10c(N)ncnc%109)ccc8C7)c(N)n6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₁H₁₃₆N₁₈O₂₀ | [M + H] = 1922.03 | [M + H] = 1922.1 |
| Example 109 | | | |
| 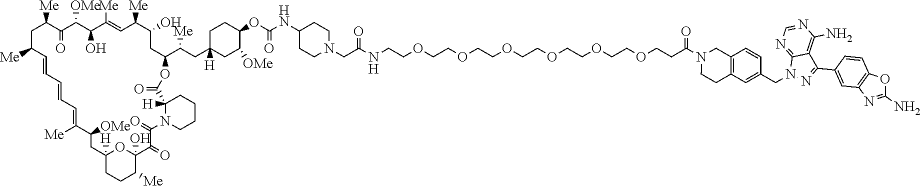 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NC3CCN(CC(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₆H₁₄₀N₁₂O₂₃ | [M + H] = 1830.02 | [M + H] = 1829.9 |
| Example 110 | | | |
| 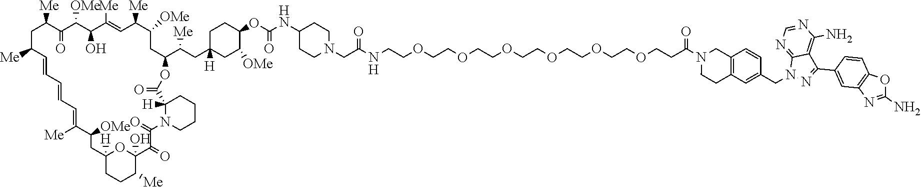 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NC3CCN(CC(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₇H₁₄₂N₁₂O₂₃ | [M + H] = 1844.04 | [M + H] = 1843.9 |
| Example 111 | | | |
| 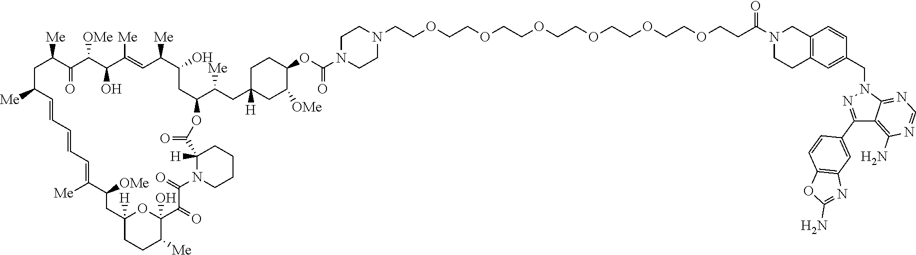 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCN(CCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₃H₁₃₅N₁₁O₂₂ | [M + H] = 1758.99 | [M + H] = 1758.9 |
| Example 149 | | | |
| 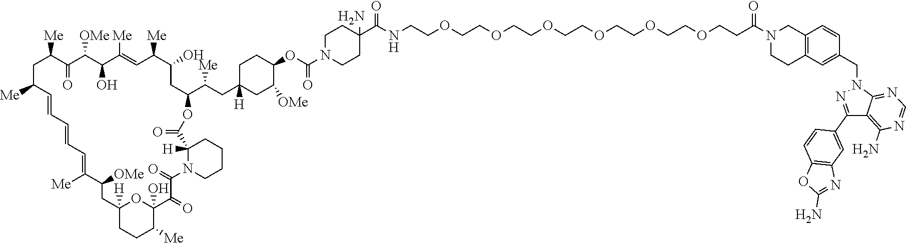 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCC(N)(C(=O)NCCOCCOCCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] | C₉₅H₁₃₈N₁₂O₂₃ | [M + H] = 1816.01 | [M + H] = 1815.9 |
| Example 150 | | | |

Following General Procedure 1, but using the appropriate amine containing active site inhibitors in Table 2 and amine containing pre-linkers in Table 4, the Intermediates C1 in Table 16 were prepared:

TABLE 16
Additional amines prepared

| | Molecular | Calculated | Observed |
|---|---|---|---|
| Structure | Formula | MW | MW |
| 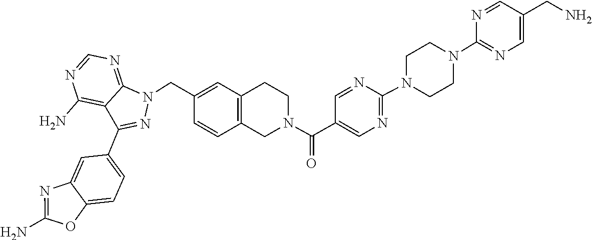 [SMILES: NCc1cnc(N2CCN(c3ncc(C(=O)N4CCc5cc(Cn6nc(-c7ccc8oc(N)nc8c7)c7c(N)ncnc76)ccc5C4)cn3)CC2)nc1] | C₃₆H₃₅N₁₅O₂ | [M + H] = 710.32 | [M + H] = 710.2 |
| Intermediate C1-1 | | | |
| 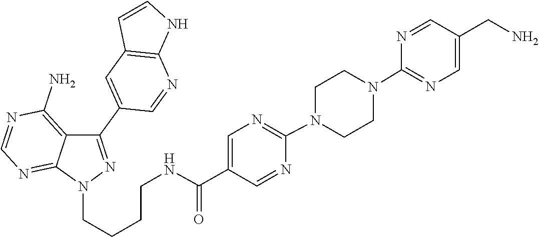 [SMILES: NCc1cnc(N2CCN(c3ncc(C(=O)NCCCCn4nc(-c5cnc6[nH]ccc6c5)c5c(N)ncnc54)cn3)CC2)nc1] | C₃₀H₃₃N₁₅O | [M + H] = 620.31 | [M + H] = 620.2 |
| Intermediate C1-2 | | | |
| 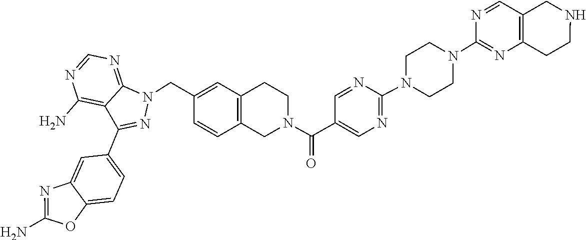 [SMILES: Nc1nc2cc(-c3nn(Cc4ccc5c(c4)CCN(C(=O)c4cnc(N6CCN(c7ncc8c(n7)CCNC8)CC6)nc4)C5)c4ncnc(N)c34)ccc2o1] | C₃₈H₃₇N₁₅O₂ | [M + H] = 736.34 | [M + H] = 736.2 |
| Intermediate C1-3 | | | |
| 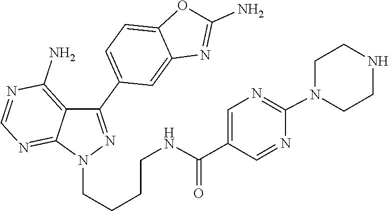 [SMILES: Nc1nc2cc(-c3nn(CCCCNC(=O)c4cnc(N5CCNCC5)nc4)c4ncnc(N)c34)ccc2o1] | C₂₅H₂₈N₁₂O₂ | [M + H] = 529.26 | [M + H] = 529.5 |
| Intermediate C1-4 | | | |
| 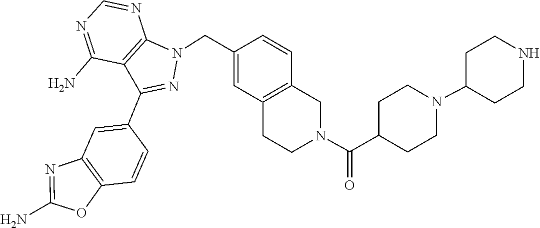 [SMILES: Nc1nc2cc(-c3nn(Cc4ccc5c(c4)CCN(C(=O)C4CCN(C6CCNCC6)CC4)C5)c4ncnc(N)c34)ccc2o1] | C₃₃H₃N₁₀O₂ | [M + H] = 607.33 | [M + H] = 607.3 |
| Intermediate C1-5 | | | |
| 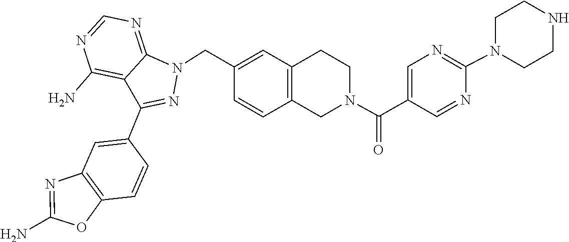 [SMILES: Nc1nc2cc(-c3nn(Cc4ccc5c(c4)CCN(C(=O)c4cnc(N6CCNCC6)nc4)C5)c4ncnc(N)c34)ccc2o1] | C₃₁H₃₀N₁₂O₂ | [M + H] = 603.27 | [M + H] = 603.3 |
| Intermediate C1-6 | | | |
| 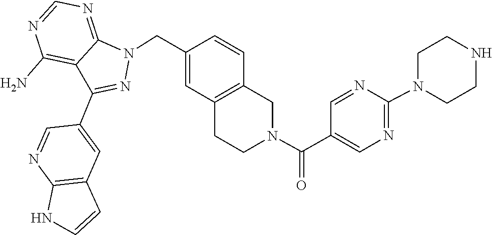 [SMILES: Nc1ncnc2c1c(-c1cnc3[nH]ccc3c1)nn2Cc1ccc2c(c1)CCN(C(=O)c1cnc(N3CCNCC3)nc1)C2] | C₃₁H₃₀N₁₂O | [M + H] = 587.28 | [M + H] = 587.3 |
| Intermediate C1-7 | | | |
| 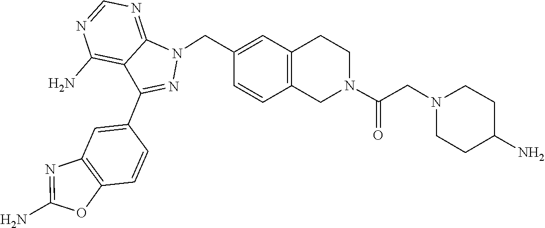 [SMILES: Nc1nc2cc(-c3nn(Cc4ccc5c(c4)CCN(C(=O)CN4CCC(N)CC4)C5)c4ncnc(N)c34)ccc2o1] | C₂₉H₃₂N₁₀O₂ | [M + H] = 553.28 | [M + H] = |
| Intermediate C1-8 | | | |

Following General Procedure 1, but using the PEG carboxylic acids and Intermediates C1 in Table 16, the Intermediates C2 in Table 17 were prepared:

TABLE 17
Additional amines prepared

| | Molecular | Calculated | Observed |
|---|---|---|---|
| Structure | Formula | MW | MW |
| 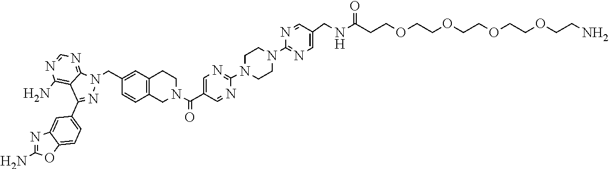 [SMILES: NCCOCCOCCOCCOCCC(=O)NCc1cnc(N2CCN(c3ncc(C(=O)N4CCc5cc(Cn6nc(-c7ccc8oc(N)nc8c7)c7c(N)ncnc76)ccc5C4)cn3)CC2)nc1] | C₄₇H₅₆N₁₆O₇ | [M + H] = 957.46 | [M + H] = 957.7 |
| Intermediate C2-1 | | | |
| 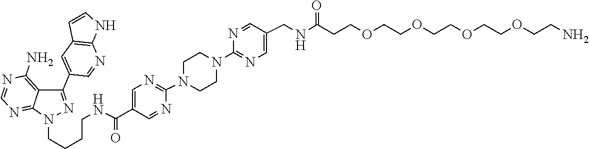 [SMILES: NCCOCCOCCOCCOCCC(=O)NCc1cnc(N2CCN(c3ncc(C(=O)NCCCCn4nc(-c5cnc6[nH]ccc6c5)c5c(N)ncnc54)cn3)CC2)nc1] | C₄₁H₅₄N₁₆O₆ | [M + H] = 867.45 | [M + H] = 867.2 |
| Intermediate C2-2 | | | |
| 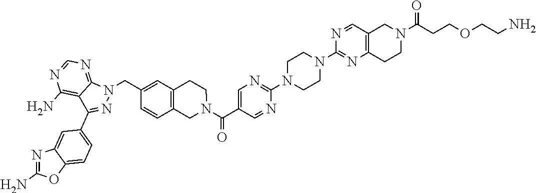 [SMILES: NCCOCCC(=O)N1CCc2nc(N3CCN(c4ncc(C(=O)N5CCc6cc(Cn7nc(-c8ccc9oc(N)nc9c8)c8c(N)ncnc87)ccc6C5)cn4)CC3)ncc2C1] | C₄₃H₄₆N₁₆O₄ | [M + H] = 851.40 | [M + H] = 851.2 |
| Intermediate C2-3 | | | |
| 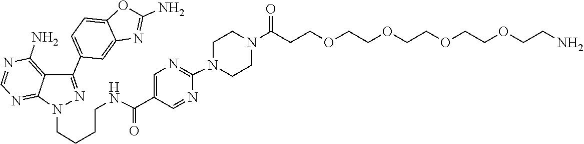 [SMILES: NCCOCCOCCOCCOCCC(=O)N1CCN(c2ncc(C(=O)NCCCCn3nc(-c4ccc5oc(N)nc5c4)c4c(N)ncnc43)cn2)CC1] | C₃₆H₄₉N₁₃O₇ | [M + H] = 776.40 | [M + H] = 776.3 |
| Intermediate C2-4 | | | |
| 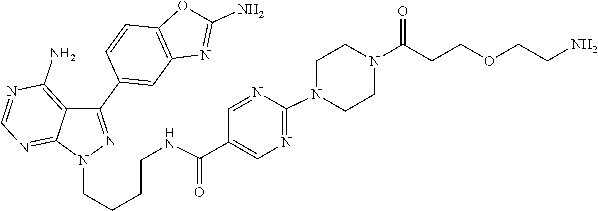 [SMILES: NCCOCCC(=O)N1CCN(c2ncc(C(=O)NCCCCn3nc(-c4ccc5oc(N)nc5c4)c4c(N)ncnc43)cn2)CC1] | C₃₀H₃₇N₁₃O₄ | [M + H] = 644.32 | [M + H] = 644.3 |
| Intermediate C2-5 | | | |
| 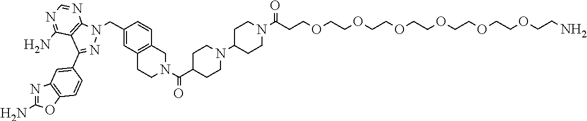 [SMILES: NCCOCCOCCOCCOCCOCCOCCC(=O)N1CCC(N2CCC(C(=O)N3CCc4cc(Cn5nc(-c6ccc7oc(N)nc7c6)c6c(N)ncnc65)ccc4C3)CC2)CC1] | C₄₈H₆₇N₁₁O₉ | [M + H] = 942.52 | [M + H] = 943.2 |
| Intermediate C2-6 | | | |
| 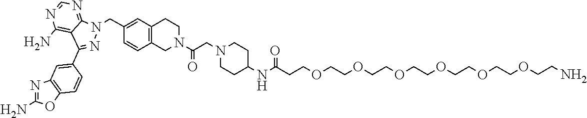 [SMILES: NCCOCCOCCOCCOCCOCCOCCC(=O)NC1CCN(CC(=O)N2CCc3cc(Cn4nc(-c5ccc6oc(N)nc6c5)c5c(N)ncnc54)ccc3C2)CC1] | C₄₄H₆₁N₁₁O₉ | [M + H] = 888.48 | [M + H] = 888.3 |
| Intermediate C2-7 | | | |

Following General Procedure 2, but using the appropriate 4-nitrophenyl carbonate containing rapamycin monomer in Table 1 and Intermediates C2 from Table 17, the Series 3 bivalent analogs in Table 18 were synthesized:

TABLE 18
Series 3 Bivalent Compounds

| | | Calcu- | Ob- |
|---|---|---|---|
| | Molecular | lated | served |
| Structure | Formula | MW | MW |
| 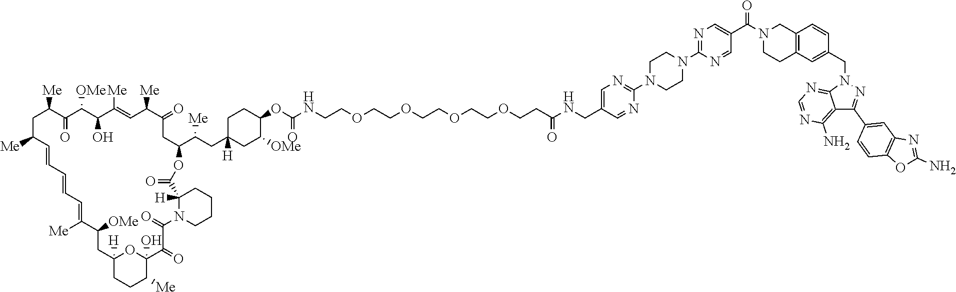 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 46 | C₉₉H₁₃₃N₁₇O₂₁ | [M + H] = 1896.99 | [M + H] = 1897.3 |
| 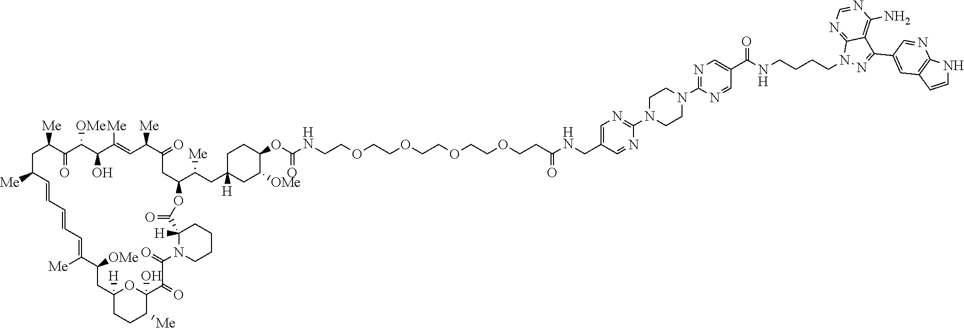 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)NCCCCn6nc(-c7cnc8[nH]ccc8c7)c7c(N)ncnc76)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 47 | C₉₃H₁₃₁N₁₇O₂₀ | [M + H] = 1806.98 | [M + H] = 1806.8 |
| 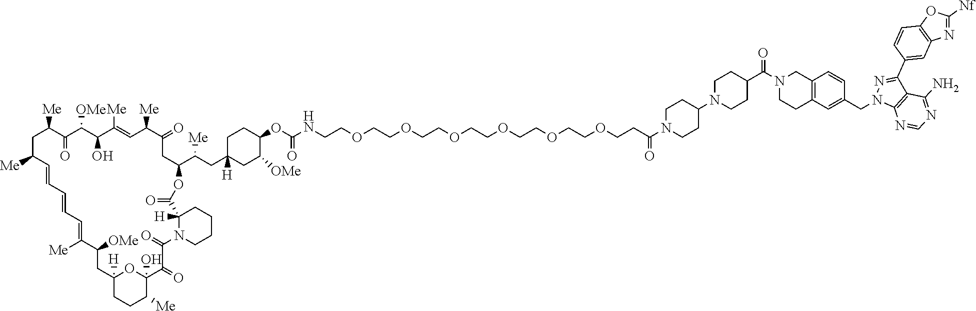 Example 48 | C₁₀₀H₁₄₄N₁₂O₂₃ | [M + H] = 1882.06 | [M + H] = 1882.1 |
| 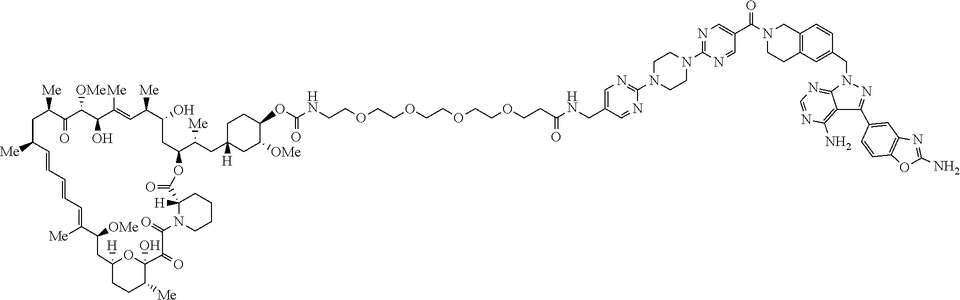 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 49 | C₉₉H₁₃₅N₁₇O₂₁ | [M + H] = 1899.01 | [M + H] = 1899.1 |
| 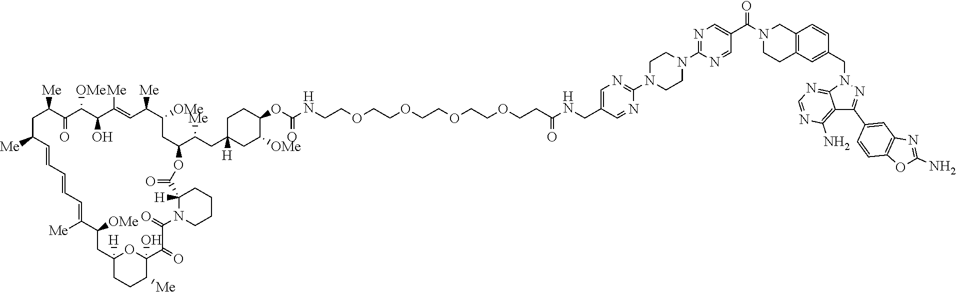 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCC(=O)NCc3cnc(N4CCN(c5ncc(C(=O)N6CCc7cc(Cn8nc(-c9ccc%10oc(N)nc%10c9)c9c(N)ncnc98)ccc7C6)cn5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 50 | C₁₀₀H₁₃₇N₁₇O₂₁ | [M + H] = 1913.03 | [M + H] = 1913.1 |
| 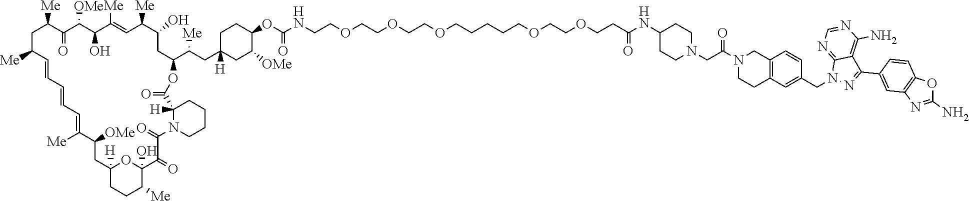 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCCCCOCCOCCC(=O)NC3CCN(CC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 112 | C₉₆H₁₄₀N₁₂O₂₃ | [M + H] = 1830.02 | [M + H] = 1829.9 |
| 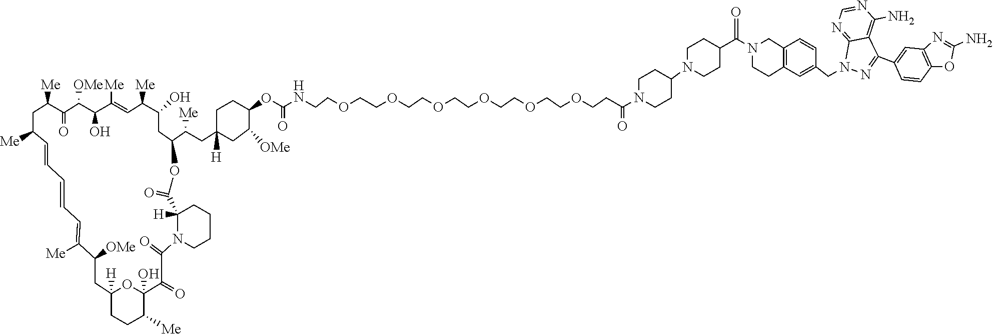 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCOCCOCCOCCOCCC(=O)N3CCC(N4CCC(C(=O)N5CCc6cc(Cn7nc(-c8ccc9oc(N)nc9c8)c8c(N)ncnc87)ccc6C5)CC4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 113 | C₁₀₀H₁₄₆N₁₂O₂₃ | [M + H] = 1884.07 | [M + H] = 1883.7 |

Following General Procedure 1, but using the appropriate Intermediates C2 in Table 17 and amine containing pre-linkers in Table 4, the Intermediates D1 in Table 19 were prepared:

TABLE 19
Additional amines prepared

| | | Calcu- | Ob- |
|---|---|---|---|
| | Molecular | lated | served |
| Structure | Formula | MW | MW |
| 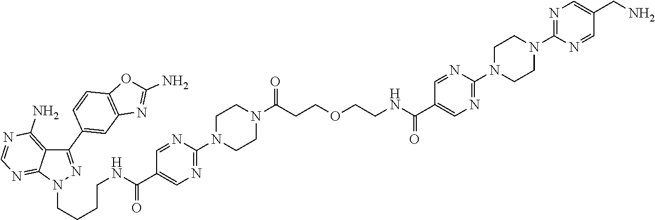 [SMILES: NCc1cnc(N2CCN(c3ncc(C(=O)NCCOCCC(=O)N4CCN(c5ncc(C(=O)NCCCCn6nc(-c7ccc8oc(N)nc8c7)c7c(N)ncnc76)cn5)CC4)cn3)CC2)nc1] Intermediate D1-1 | C₄₄H₅₂N₂₀O₅ | [M + H] = 941.45 | [M + H] = 941.5 |

Following General Procedure 1, but using the appropriate amine containing active site inhibitors in Table 2 and amine containing pre-linkers in Table 4, the Intermediates D1 in Table 20 were prepared:

TABLE 20
Additional amines prepared

| | Molecular | Calculated | Observed |
|---|---|---|---|
| Structure | Formula | MW | MW |
| 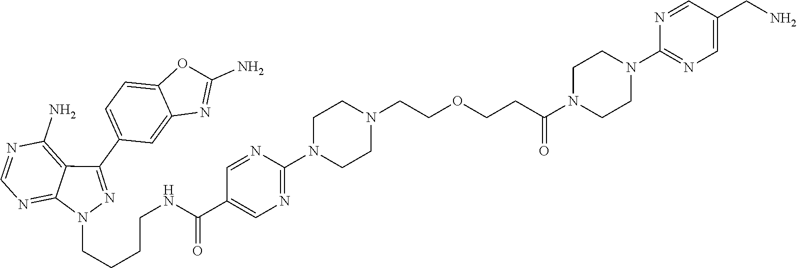 [SMILES: NCc1cnc(N2CCN(C(=O)CCOCCN3CCN(c4ncc(C(=O)NCCCCn5nc(-c6ccc7oc(N)nc7c6)c6c(N)ncnc65)cn4)CC3)CC2)nc1] Intermediate D1-2 | C₃₉H₄₉N₁₇O₄ | [M + H] = 820.43 | [M + H] = 820.4 |
| 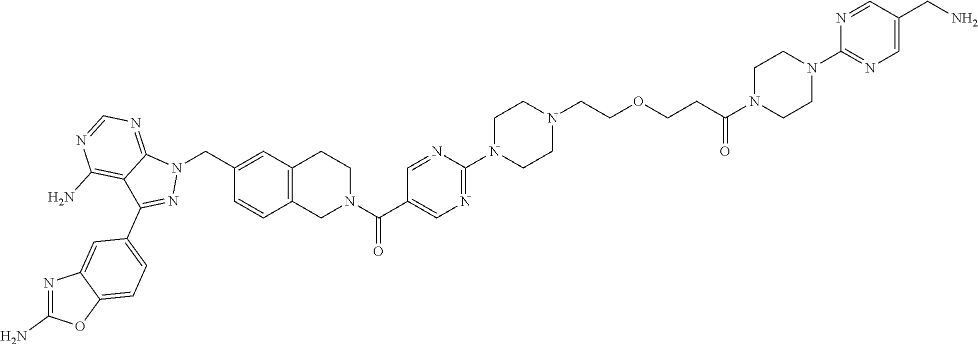 [SMILES: NCc1cnc(N2CCN(C(=O)CCOCCN3CCN(c4ncc(C(=O)N5CCc6cc(Cn7nc(-c8ccc9oc(N)nc9c8)c8c(N)ncnc87)ccc6C5)cn4)CC3)CC2)nc1] Intermediate D1-3 | C₄₅H₅₁N₁₇O₄ | [M + H] = 894.44 | [M + H] = 894.4 |
| 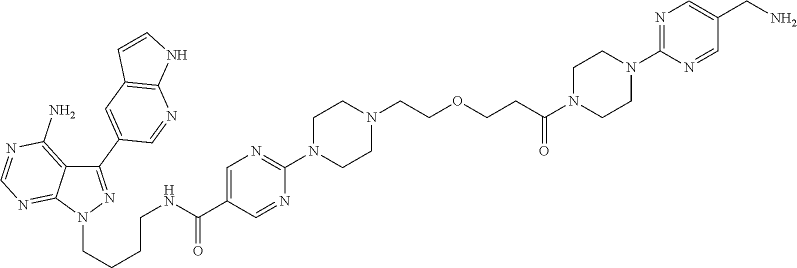 [SMILES: NCc1cnc(N2CCN(C(=O)CCOCCN3CCN(c4ncc(C(=O)NCCCCn5nc(-c6cnc7[nH]ccc7c6)c6c(N)ncnc65)cn4)CC3)CC2)nc1] Intermediate D1-4 | C₃₉H₄₉N₁₇O₃ | [M + H] = 804.43 | [M + H] = 804.4 |
| 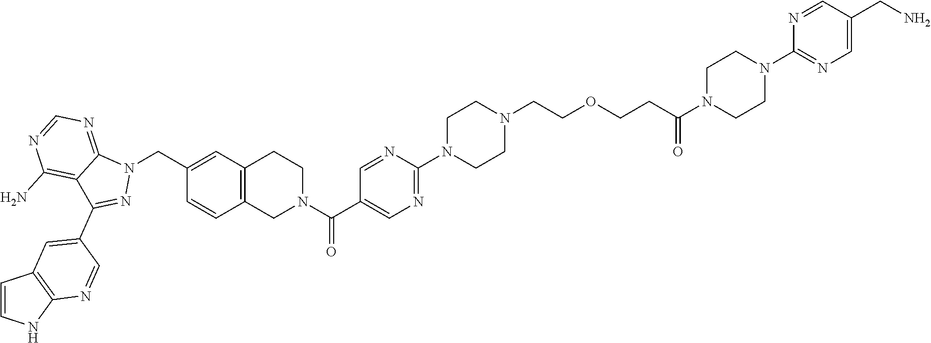 [SMILES: NCc1cnc(N2CCN(C(=O)CCOCCN3CCN(c4ncc(C(=O)N5CCc6cc(Cn7nc(-c8cnc9[nH]ccc9c8)c8c(N)ncnc87)ccc6C5)cn4)CC3)CC2)nc1] Intermediate D1-5 | C₄₅H₅₁N₁₇O₃ | [M + H] = 878.45 | [M + H] = 878.4 |
| 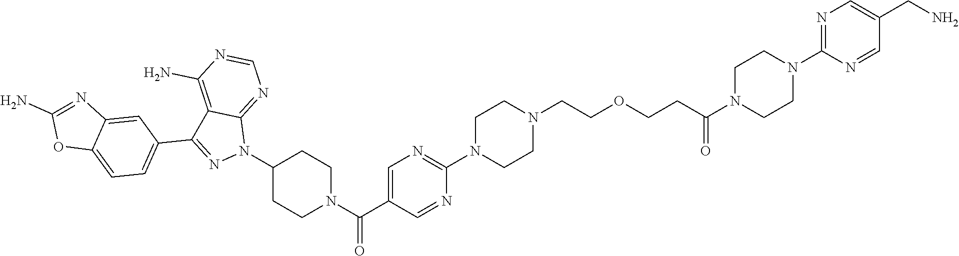 [SMILES: NCc1cnc(N2CCN(C(=O)CCOCCN3CCN(c4ncc(C(=O)N5CCC(n6nc(-c7ccc8oc(N)nc8c7)c7c(N)ncnc76)CC5)cn4)CC3)CC2)nc1] Intermediate D1-6 | C₄₀H₄₉N₁₇O₄ | [M + H] = 832.43 | [M + H] = 832.4 |
| 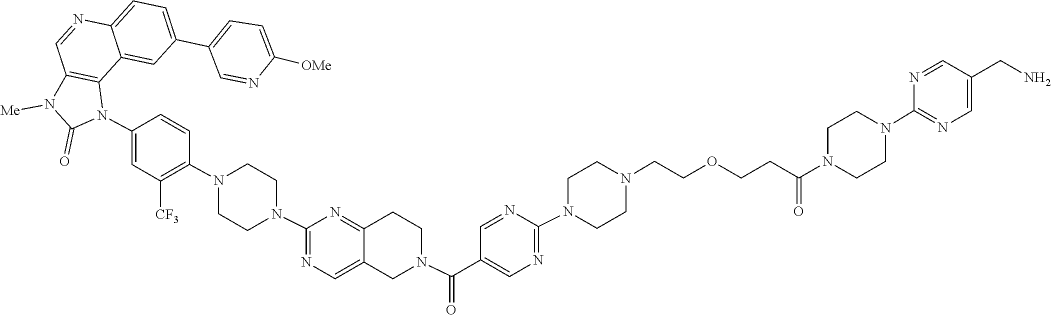 [SMILES: COc1ccc(-c2ccc3ncc4c(c3c2)n(-c2ccc(N3CCN(c5ncc6c(n5)CCN(C(=O)c5cnc(N7CCN(CCOCCC(=O)N8CCN(c9ncc(CN)cn9)CC8)CC7)nc5)C6)CC3)c(C(F)(F)F)c2)c(=O)n4C)cn1] Intermediate D1-7 | C₅₈H₆₃F₃N₁₈O₅ | [M + H] = 1149.53 | [M + H] = 1149.4 |
| 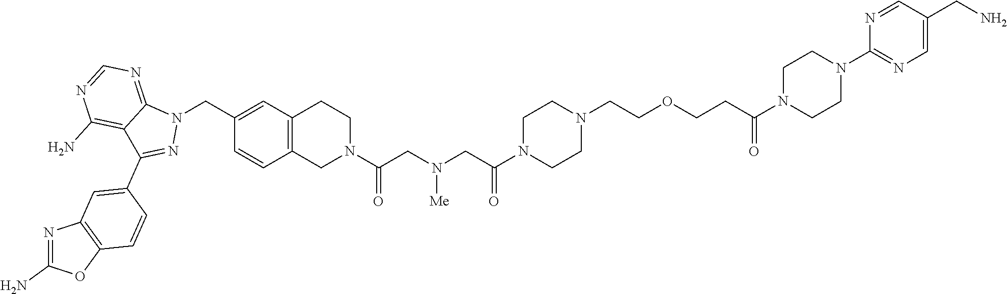 [SMILES: CN(CC(=O)N1CCN(CCOCCC(=O)N2CCN(c3ncc(CN)cn3)CC2)CC1)CC(=O)N1CCc2cc(Cn3nc(-c4ccc5oc(N)nc5c4)c4c(N)ncnc43)ccc2C1] Intermediate D1-8 | C₄₅H₅₆N₁₆O₅ | [M + H] = 901.47 | [M + H] = 901.4 |
| 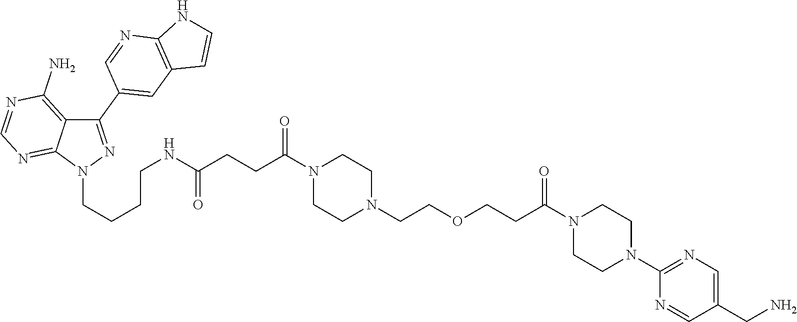 [SMILES: NCc1cnc(N2CCN(C(=O)CCOCCN3CCN(C(=O)CCC(=O)NCCCCn4nc(-c5cnc6[nH]ccc6c5)c5c(N)ncnc54)CC3)CC2)nc1] Intermediate D1-9 | C₃₈H₅₁N₁₅O₄ | [M + H] = 782.43 | [M + H] = 782.4 |
| 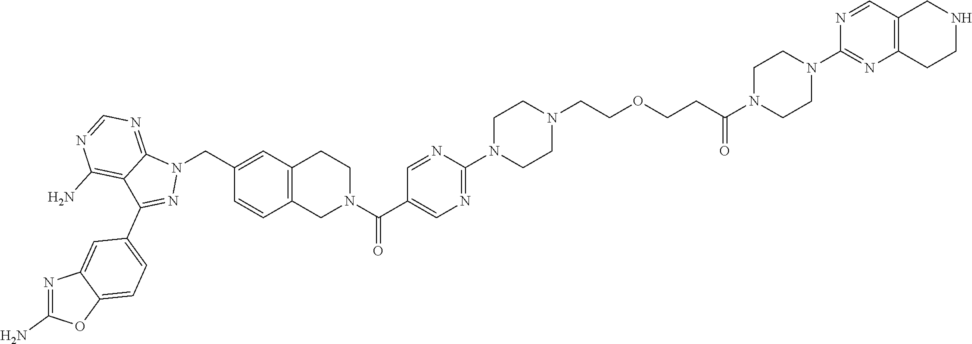 [SMILES: Nc1nc2cc(-c3nn(Cc4ccc5c(c4)CCN(C(=O)c4cnc(N6CCN(CCOCCC(=O)N7CCN(c8ncc9c(n8)CCNC9)CC7)CC6)nc4)C5)c4ncnc(N)c34)ccc2o1] Intermediate D1-10 | C₄₇H₅₃N₁₇O₄ | [M + H] = 920.46 | [M + H] = 920.4 |
| 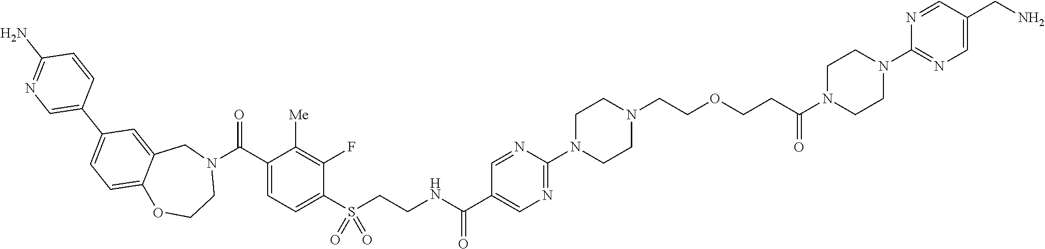 [SMILES: Cc1c(C(=O)N2CCOc3ccc(-c4ccc(N)nc4)cc3C2)ccc(S(=O)(=O)CCNC(=O)c2cnc(N3CCN(CCOCCC(=O)N4CCN(c5ncc(CN)cn5)CC4)CC3)nc2)c1F] Intermediate D1-11 | C₄₇H₅₆FN₁₃O₇S | [M + H] = 966.42 | [M + H] = 966.3 |
| 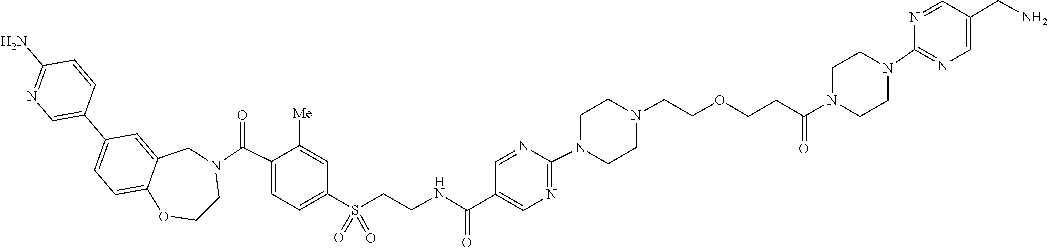 [SMILES: Cc1cc(S(=O)(=O)CCNC(=O)c2cnc(N3CCN(CCOCCC(=O)N4CCN(c5ncc(CN)cn5)CC4)CC3)nc2)ccc1C(=O)N1CCOc2ccc(-c3ccc(N)nc3)cc2C1] Intermediate D1-12 | C₄₇H₅₇N₁₃O₇S | [M + H] = 948.43 | [M + H] = 948.4 |
| 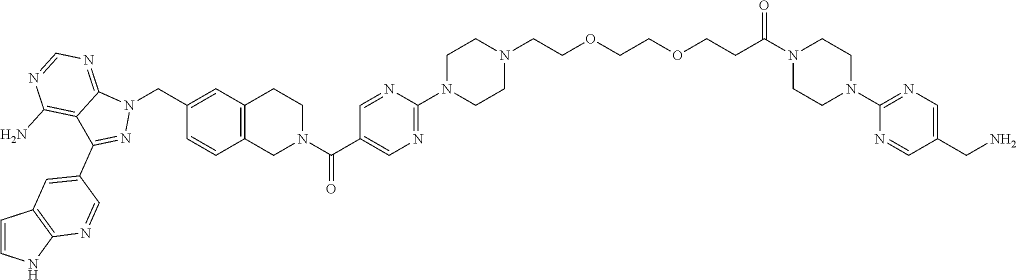 [SMILES: NCc1cnc(N2CCN(C(=O)CCOCCOCCN3CCN(c4ncc(C(=O)N5CCc6cc(Cn7nc(-c8cnc9[nH]ccc9c8)c8c(N)ncnc87)ccc6C5)cn4)CC3)CC2)nc1] Intermediate D1-13 | C₄₇H₅₅N₁₇O₄ | [M + H] = 922.47 | [M + H] = 922.4 |
| 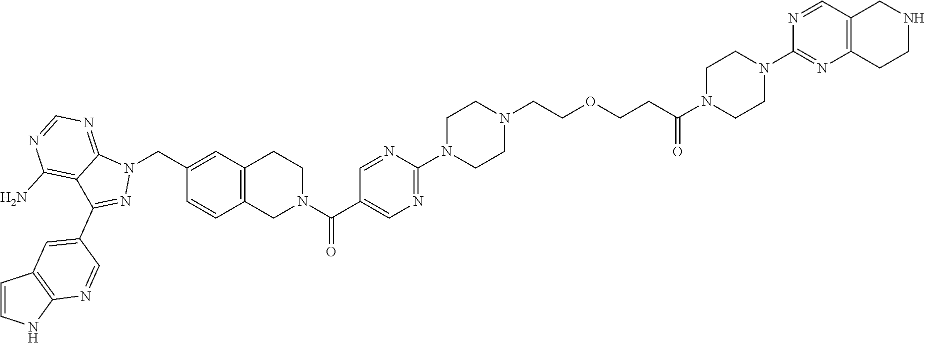 [SMILES: Nc1ncnc2c1c(-c1cnc3[nH]ccc3c1)nn2Cc1ccc2c(c1)CCN(C(=O)c1cnc(N3CCN(CCOCCC(=O)N4CCN(c5ncc6c(n5)CCNC6)CC4)CC3)nc1)C2] Intermediate D1-14 | C₄₇H₅₃N₁₇O₃ | [M + H] = 904.46 | [M + H] = 904.4 |
| 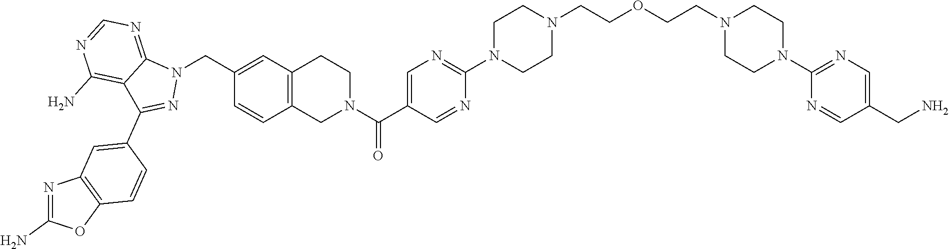 [SMILES: NCc1cnc(N2CCN(CCOCCN3CCN(c4ncc(C(=O)N5CCc6cc(Cn7nc(-c8ccc9oc(N)nc9c8)c8c(N)ncnc87)ccc6C5)cn4)CC3)CC2)nc1] Intermediate D1-15 | C₄₄H₅₁N₁₇O₃ | [M + H] = 866.45 | [M + H] = 866.3 |
| 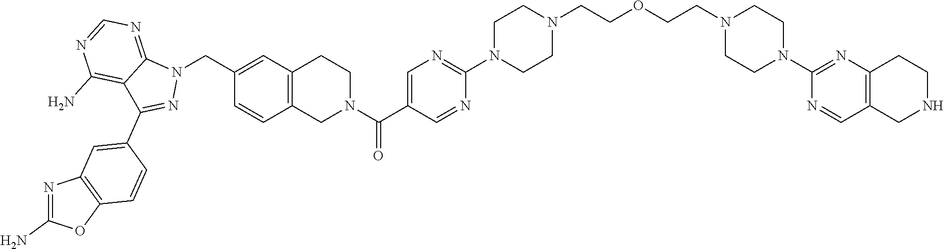 [SMILES: Nc1nc2cc(-c3nn(Cc4ccc5c(c4)CCN(C(=O)c4cnc(N6CCN(CCOCCN7CCN(c8ncc9c(n8)CCNC9)CC7)CC6)nc4)C5)c4ncnc(N)c34)ccc2o1] Intermediate D1-16 | C₄₆H₅₃N₁₇O₃ | [M + H] = 892.46 | [M + H] = 892.3 |
| 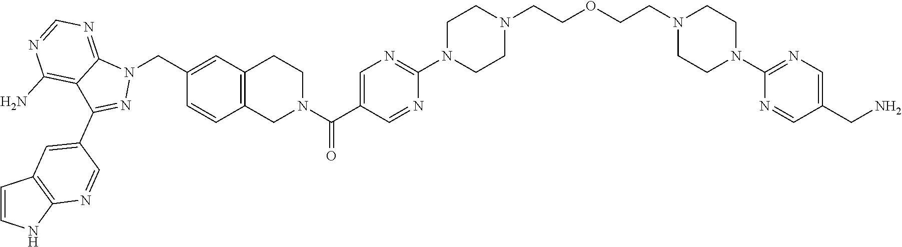 [SMILES: NCc1cnc(N2CCN(CCOCCN3CCN(c4ncc(C(=O)N5CCc6cc(Cn7nc(-c8cnc9[nH]ccc9c8)c8c(N)ncnc87)ccc6C5)cn4)CC3)CC2)nc1] Intermediate D1-17 | C₄₄H₅₁N₁₇O₂ | [M + H] = 850.45 | [M + H] = 850.3 |
| 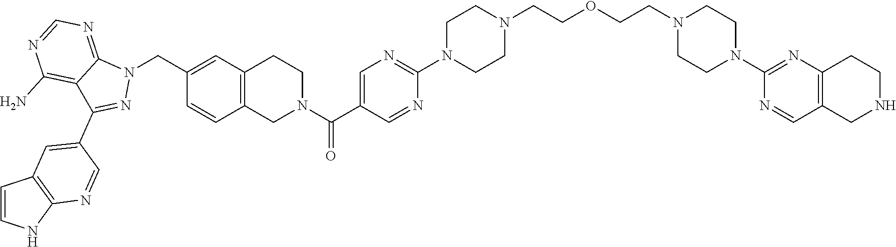 [SMILES: Nc1ncnc2c1c(-c1cnc3[nH]ccc3c1)nn2Cc1ccc2c(c1)CCN(C(=O)c1cnc(N3CCN(CCOCCN4CCN(c5ncc6c(n5)CCNC6)CC4)CC3)nc1)C2] Intermediate D1-18 | C₄₆H₅₃N₁₇O₂ | [M + H] = 876.47 | [M + H] = 876.3 |
| 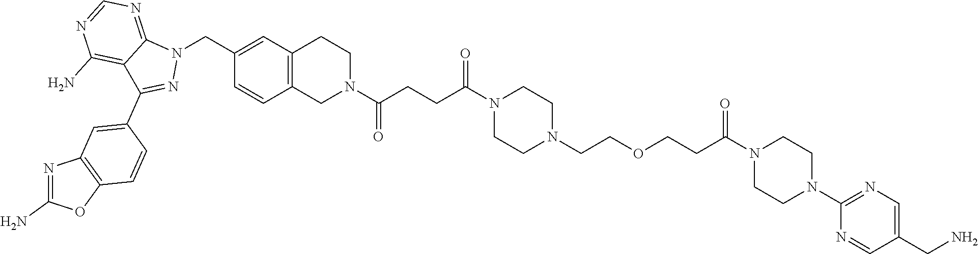 [SMILES: NCc1cnc(N2CCN(C(=O)CCOCCN3CCN(C(=O)CCC(=O)N4CCc5cc(Cn6nc(-c7ccc8oc(N)nc8c7)c7c(N)ncnc76)ccc5C4)CC3)CC2)nc1] Intermediate D1-19 | C₄₄H₅₃N₁₅O₅ | [M + H] = 872.45 | [M + H] = 872.3 |
| 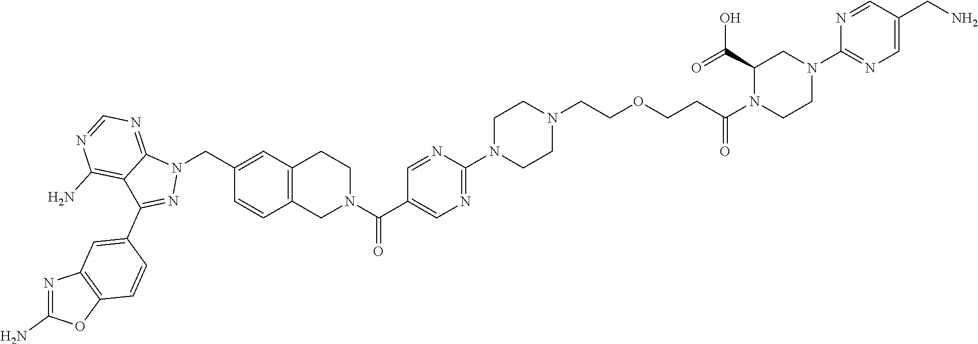 [SMILES: NCc1cnc(N2CCN(C(=O)CCOCCN3CCN(c4ncc(C(=O)N5CCc6cc(Cn7nc(-c8ccc9oc(N)nc9c8)c8c(N)ncnc87)ccc6C5)cn4)CC3)[C@@H](C(=O)O)C2)nc1] Intermediate D1-20 | C₄₆H₅₁N₁₇O₆ | [M + H] = 938.61 | [M + H] = 938.3 |
| 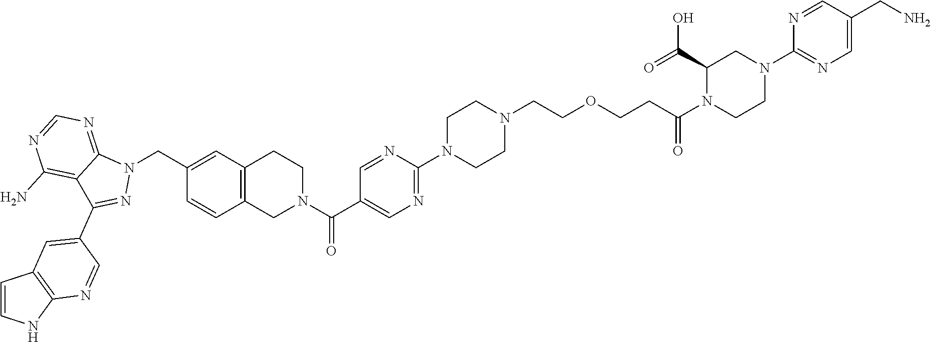 [SMILES: NCc1cnc(N2CCN(C(=O)CCOCCN3CCN(c4ncc(C(=O)N5CCc6cc(Cn7nc(-c8cnc9[nH]ccc9c8)c8c(N)ncnc87)ccc6C5)cn4)CC3)[C@@H](C(=O)O)C2)nc1] Intermediate D1-21 | C₄₆H₅₁N₁₇O₅ | [M + H] = 922.44 | [M + H] = 922.3 |
| 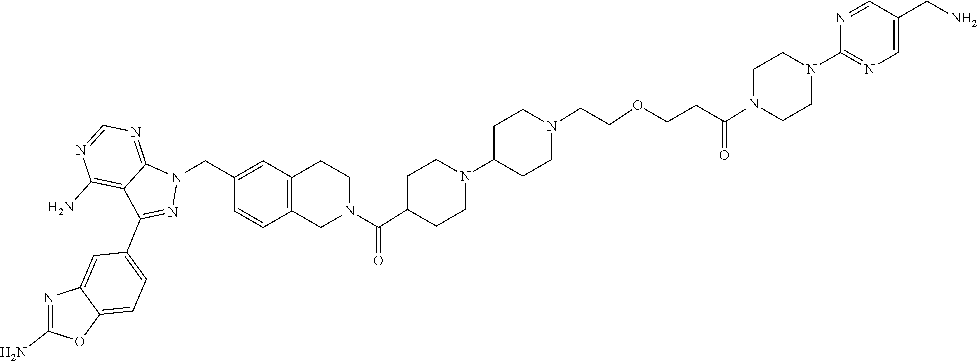 [SMILES: NCc1cnc(N2CCN(C(=O)CCOCCN3CCC(N4CCC(C(=O)N5CCc6cc(Cn7nc(-c8ccc9oc(N)nc9c8)c8c(N)ncnc87)ccc6C5)CC4)CC3)CC2)nc1] Intermediate D1-22 | C₄₇H₅₉N₁₅O₄ | [M + H] = 898.50 | [M + H] = 898.4 |

Following General Procedure 2, but using the appropriate 4-nitrophenyl carbonate containing rapamycin monomer in Table 1 and Intermediates D1 from Tables 19 and 20, the Series 4 bivalent analogs in Table 21 were synthesized:

TABLE 21
Series 4 Bivalent Compounds

| | | Calcu- | Ob- |
|---|---|---|---|
| | Molecular | lated | served |
| Structure | Formula | MW | MW |
| 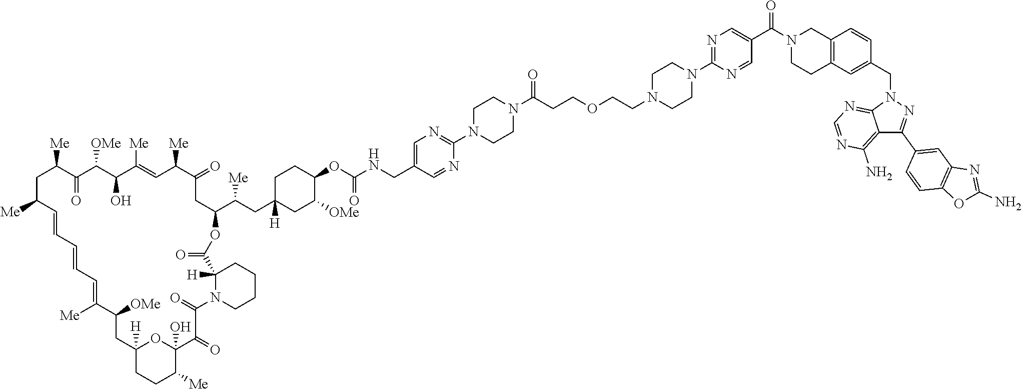 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 51 | C₉₇H₁₂₈N₁₈O₁₈ | [M + H] = 1833.98 | [M + H] = 1834.1 |
| 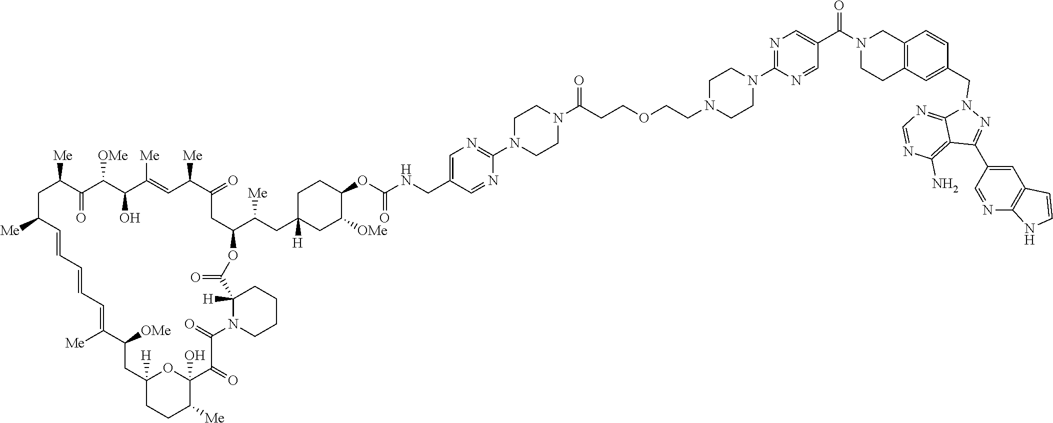 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 52 | C₉₇H₁₂₈N₁₈O₁₇ | [M + H] = 1817.98 | [M + H] = 1817.9 |
| 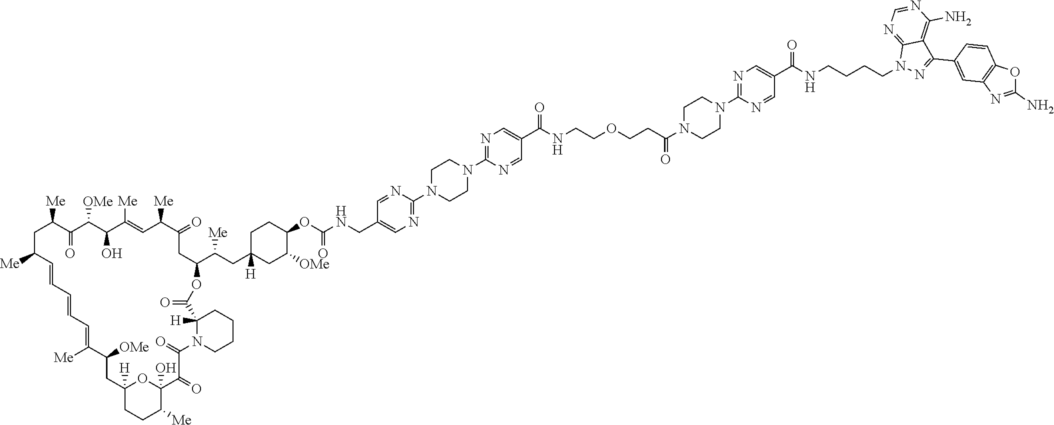 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)NCCOCCC(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 53 | C₉₆H₁₂₉N₂₁O₁₉ | [M + H] = 1880.99 | [M + H] = 1881.0 |
| 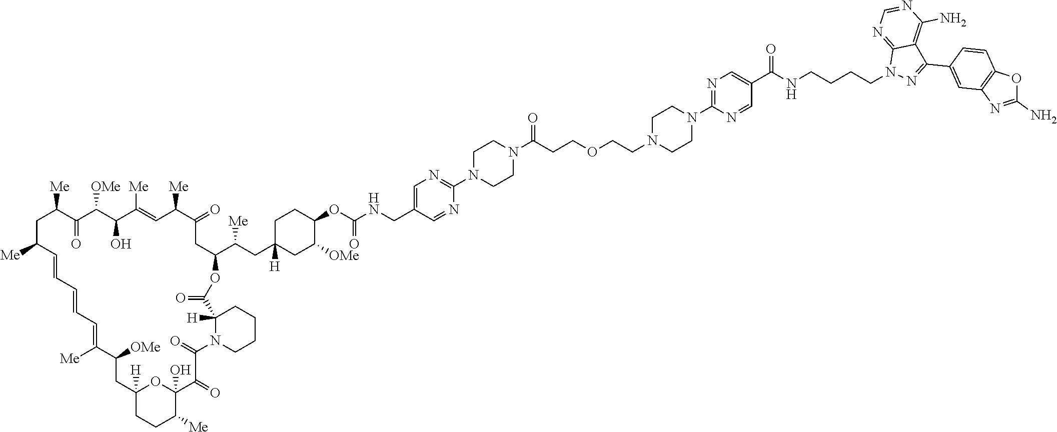 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 54 | C₉₁H₁₂₆N₁₈O₁₈ | [M + H] = 1759.96 | [M + H] = 1760.0 |
| 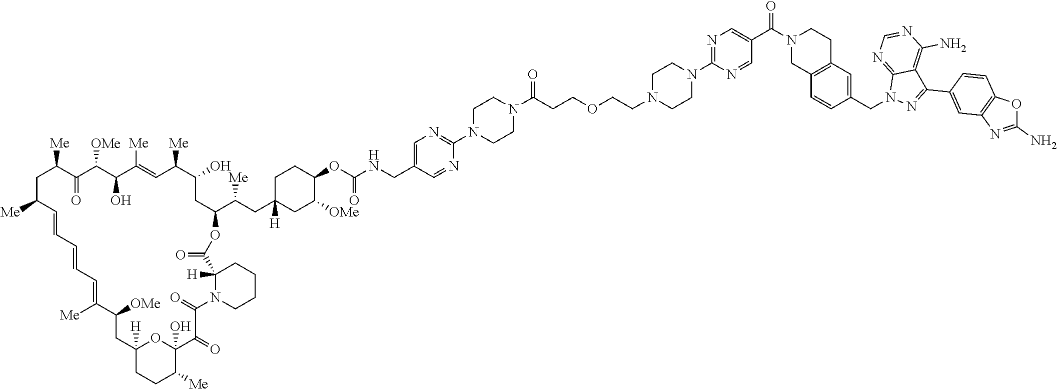 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 55 | C₉₇H₁₃₀N₁₈O₁₈ | [M + H] = 1835.99 | [M + H] = 1835.8 |
| 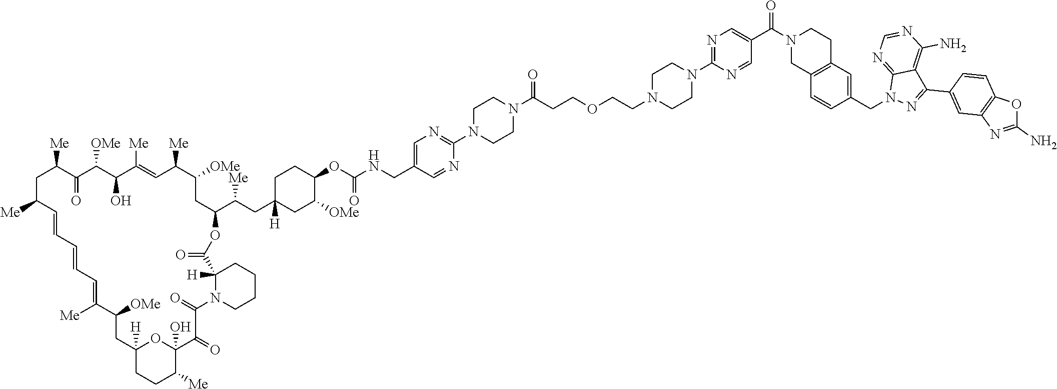 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 56 | C₉₈H₁₃₂N₁₈O₁₈ | [M + H] = 1850.01 | [M + H] = 1849.8 |
| 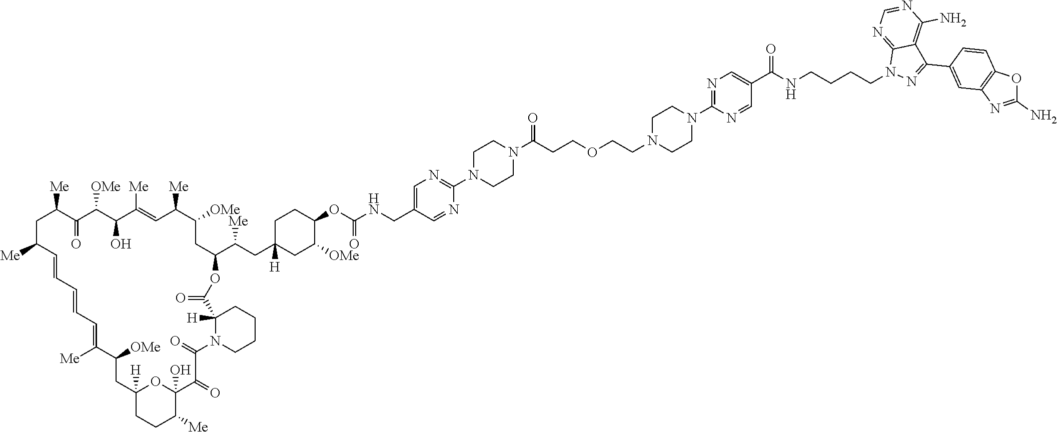 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCCCn7nc(-c8ccc9oc(N)nc9c8)c8c(N)ncnc87)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 57 | C₉₂H₁₃₀N₁₈O₁₈ | [M + H] = 1775.99 | [M + H] = 1775.9 |
| 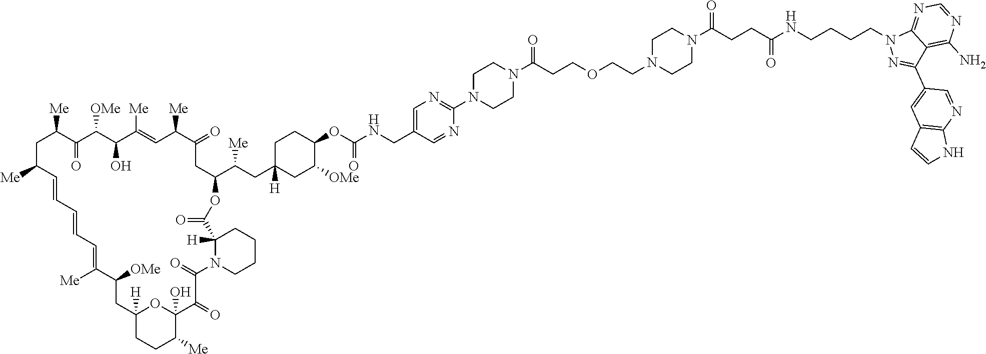 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)NCCCCn6nc(-c7cnc8[nH]ccc8c7)c7c(N)ncnc76)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 58 | C₉₀H₁₂₈N₁₆O₁₈ | [M + H] = 1721.97 | [M + H] = 1721.9 |
| 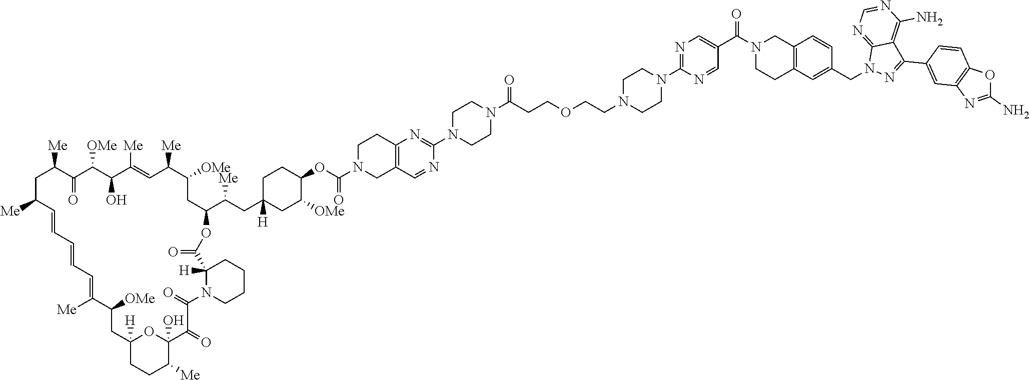 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ncc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 114 | C₁₀₀H₁₃₄N₁₈O₁₈ | [M + H] = 1876.02 | [M + H] = 1876.1 |
| 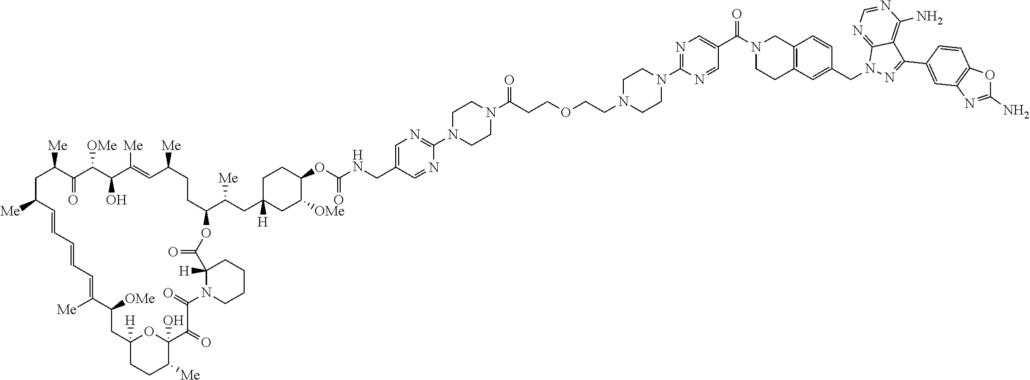 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC[C@H](C)/C=C(\C)[C@@H](O)[C@@H](OC)C(=O)[C@H](C)C[C@H](C)/C=C/C=C/C=C/1C] Example 115 | C₉₇H₁₃₀N₁₈O₁₇ | [M + H] = 1819.99 | [M + H] = 1820.1 |
| 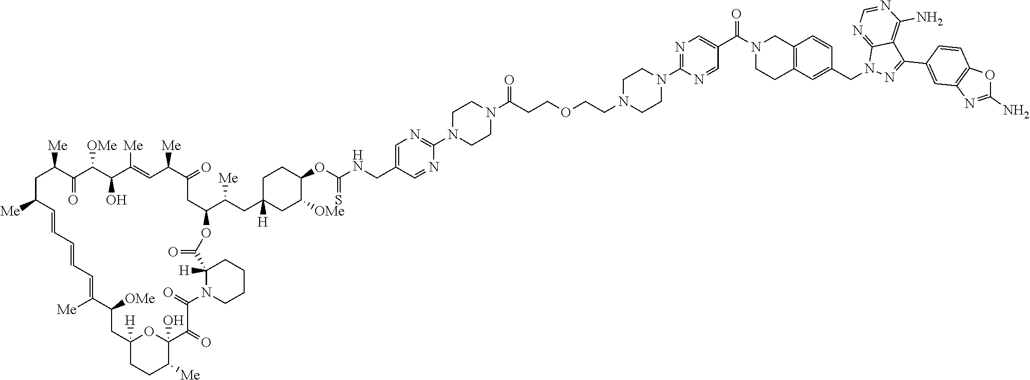 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 116 | C₉₇H₁₂₈N₁₈O₁₇S | [M + H] = 1849.95 | [M + H] = 1849.9 |
| 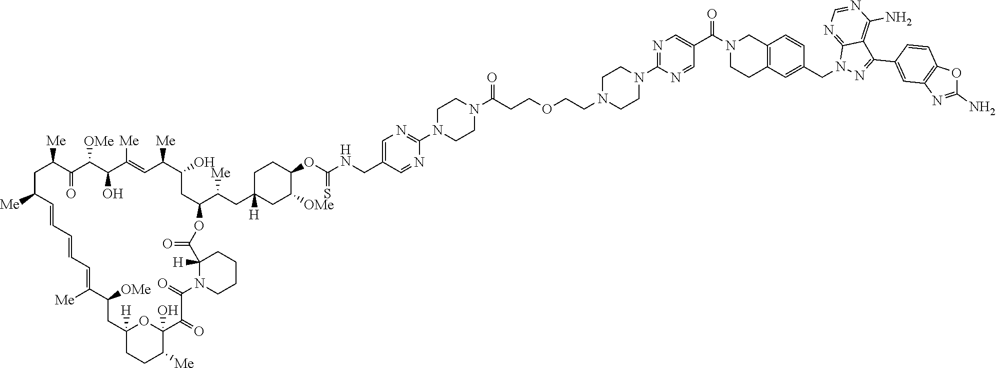 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 117 | C₉₇H₁₃₀N₁₈O₁₇S | [M + H] = 1851.97 | [M + H] = 1851.9 |
| 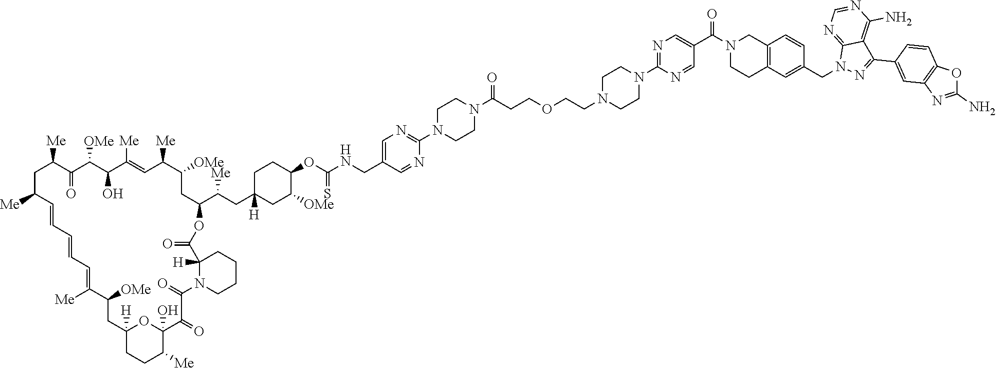 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=S)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 118 | C₉₈H₁₃₂N₁₈O₁₇S | [M + H] = 1865.98 | [M + H] = 1865.7 |
| 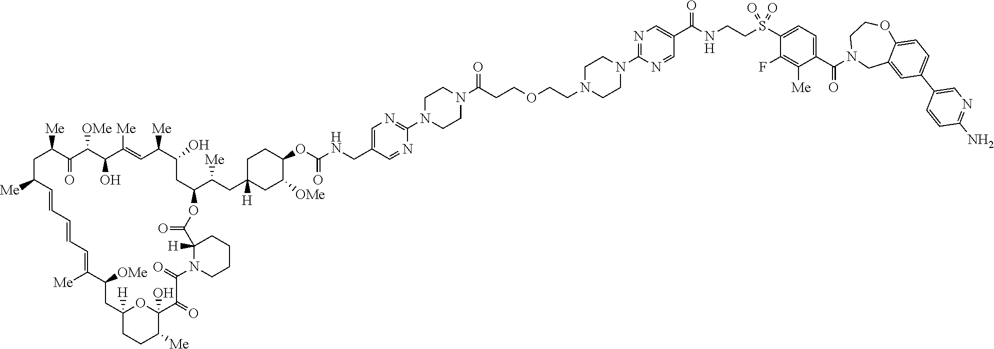 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7F)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 119 | C₉₉H₁₃₅FN₁₄O₂₁S | [M + H] = 1907.97 | [M + H] = 1908.0 |
| 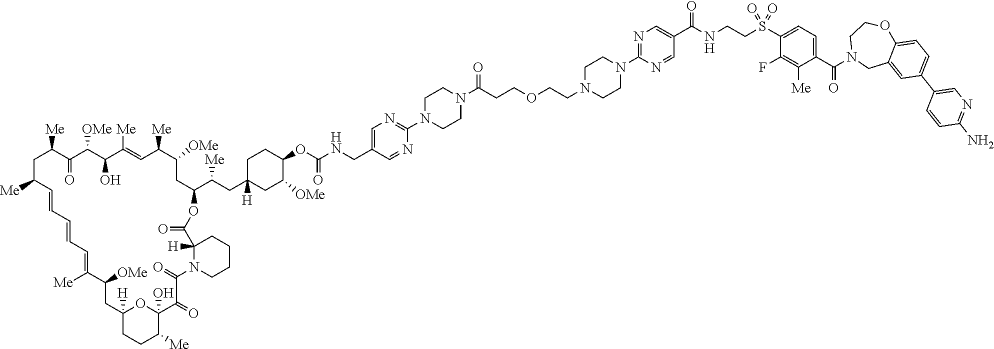 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7F)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 120 | C₁₀₀H₁₃₇FN₁₄O₂₁S | [M + H] = 1921.99 | [M + H] = 1922.0 |
| 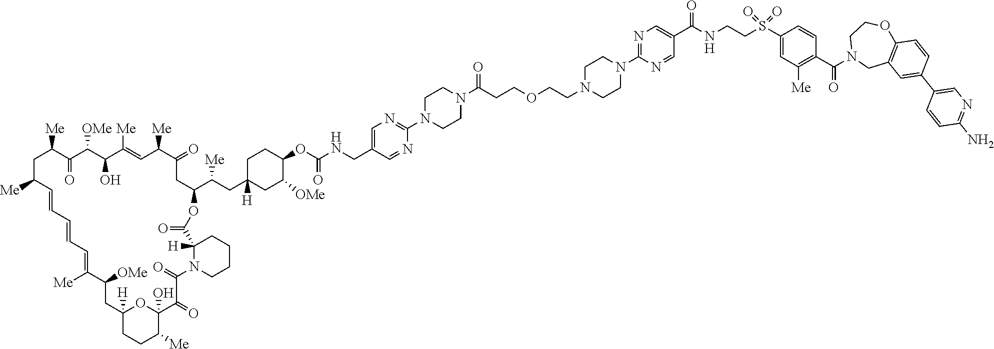 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 121 | C₉₉H₁₃₄N₁₄O₂₁S | [M + H] = 1887.96 | [M + H] = 1888.0 |
| 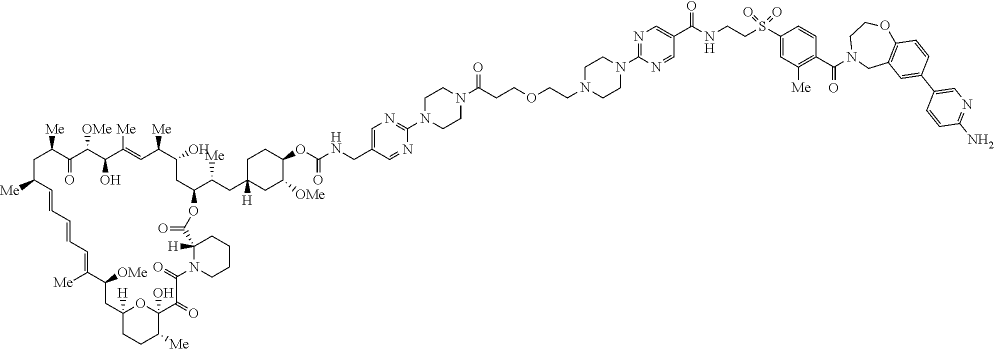 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCS(=O)(=O)c7ccc(C(=O)N8CCOc9ccc(-c%10ccc(N)nc%10)cc9C8)c(C)c7)cn6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 122 | C₉₉H₁₃₆N₁₄O₂₁S | [M + H] = 1889.98 | [M + H] = 1890.0 |
| 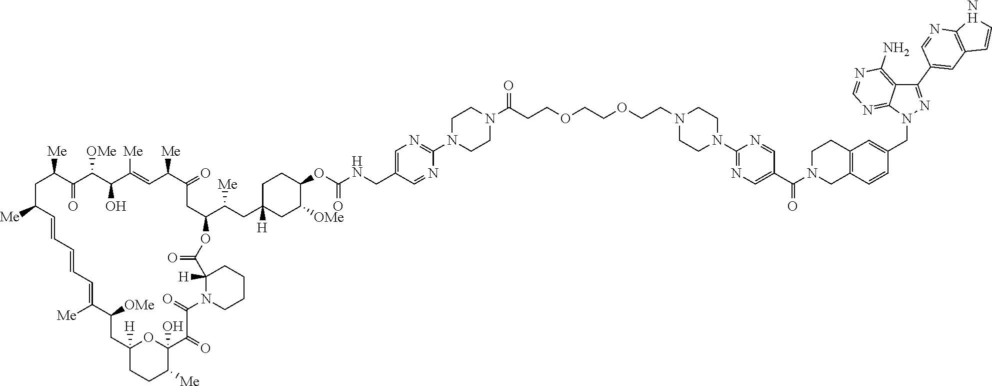 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 123 | C₉₉H₁₃₂N₁₈O₁₈ | [M + H] = 1862.00 | [M + H] = 1861.9 |
| 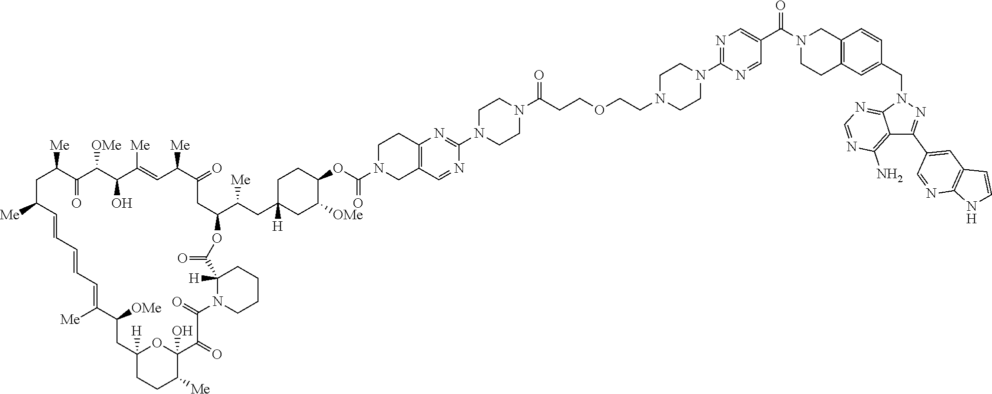 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(C(=O)CCOCCN6CCN(c7ncc(C(=O)N8CCc9cc(Cn%10nc(-c%11cnc%12[nH]ccc%12c%11)c%11c(N)ncnc%11%10)ccc9C8)cn7)CC6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 124 | C₉₉H₁₃₀N₁₈O₁₇ | [M + H] = 1843.99 | [M + H] = 1844.1 |
| 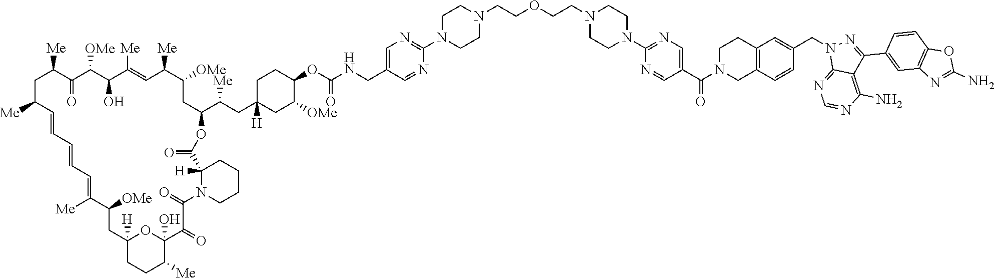 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 125 | C₉₇H₁₃₂N₁₈O₁₇ | [M + H] = 1822.01 | [M + H] = 1822.0 |
| 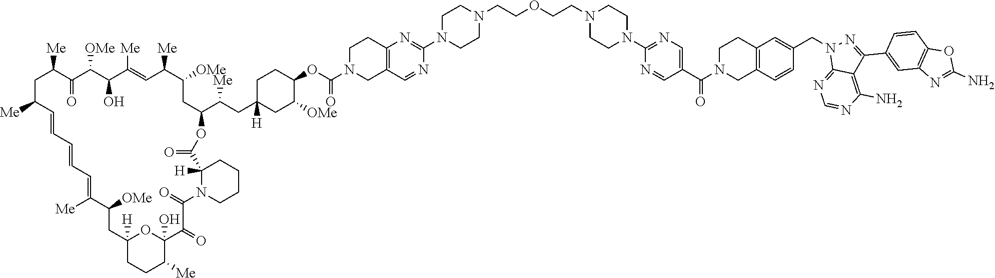 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCN6CCN(c7ncc(C(=O)N8CCc9cc(Cn%10nc(-c%11ccc%12oc(N)nc%12c%11)c%11c(N)ncnc%11%10)ccc9C8)cn7)CC6)CC5)ncc4C3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 126 | C₉₉H₁₃₄N₁₈O₁₇ | [M + H] = 1848.03 | [M + H] = 1848.0 |
| 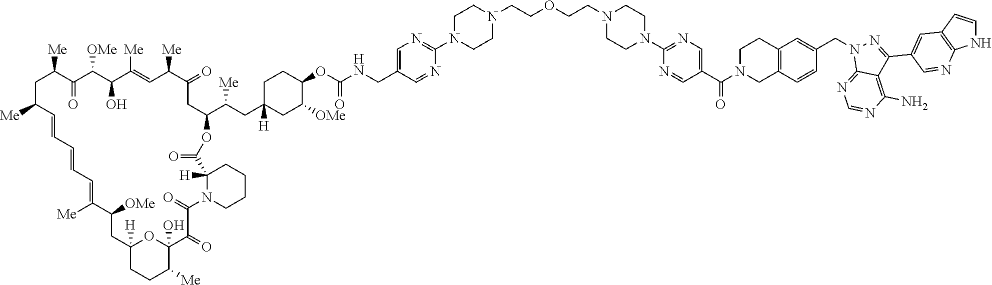 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(CCOCCN5CCN(c6ncc(C(=O)N7CCc8cc(Cn9nc(-c%10cnc%11[nH]ccc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 127 | C₉₆H₁₂₈N₁₈O₁₆ | [M + H] = 1789.98 | [M + H] = 1789.9 |
| 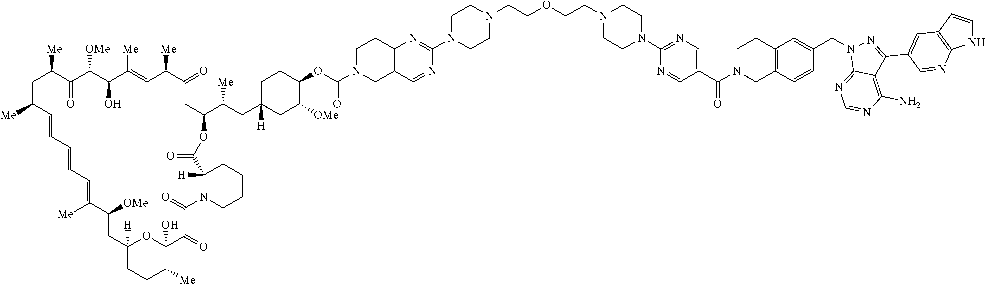 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(CCOCCN6CCN(c7ncc(C(=O)N8CCc9cc(Cn%10nc(-c%11cnc%12[nH]ccc%12c%11)c%11c(N)ncnc%11%10)ccc9C8)cn7)CC6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 128 | C₉₈H₁₃₀N₁₈O₁₆ | [M + H] = 1816.00 | [M + H] = 1815.9 |
| 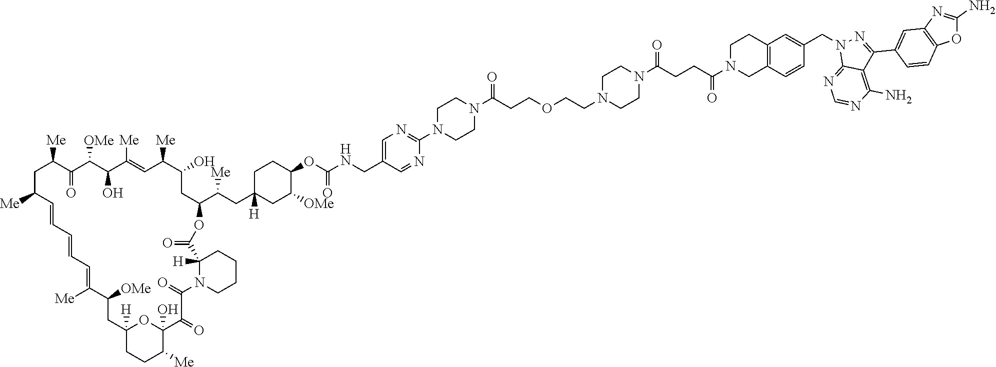 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(C(=O)CCC(=O)N6CCc7cc(Cn8nc(-c9ccc%10oc(N)nc%10c9)c9c(N)ncnc98)ccc7C6)CC5)CC4)nc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 129 | C₉₆H₁₃₂N₁₆O₁₉ | [M + H] = 1813.99 | [M + H] = 1814.0 |
| 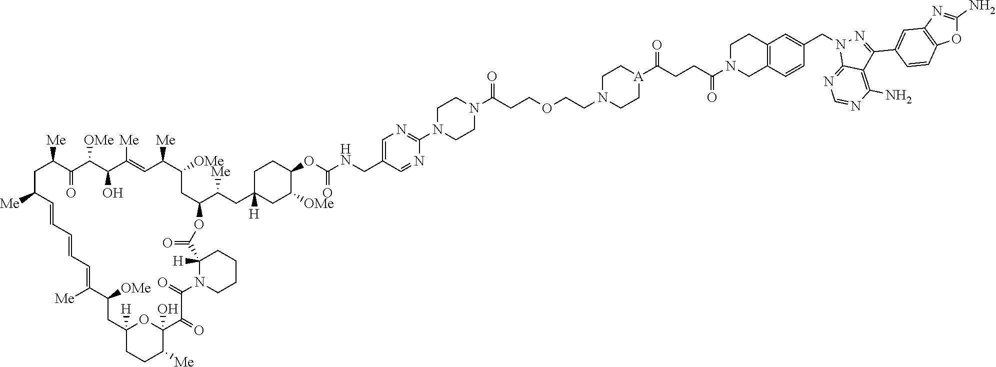 Example 130 | C₉₇H₁₃₄N₁₆O₁₉ | [M + H] = 1828.01 | [M + H] = 1828.0 |
| 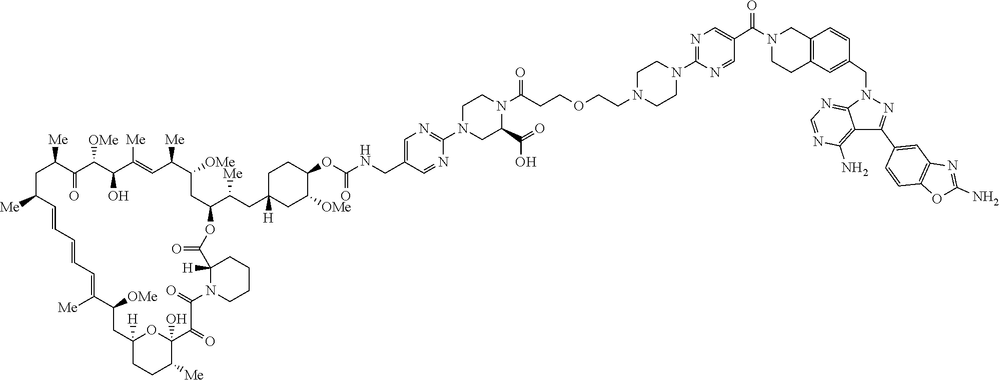 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)[C@@H](C(=O)O)C4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 131 | C₉₉H₁₃₂N₁₈O₂₀ | [M + H] = 1893.99 | [M + H] = 1894.0 |
| 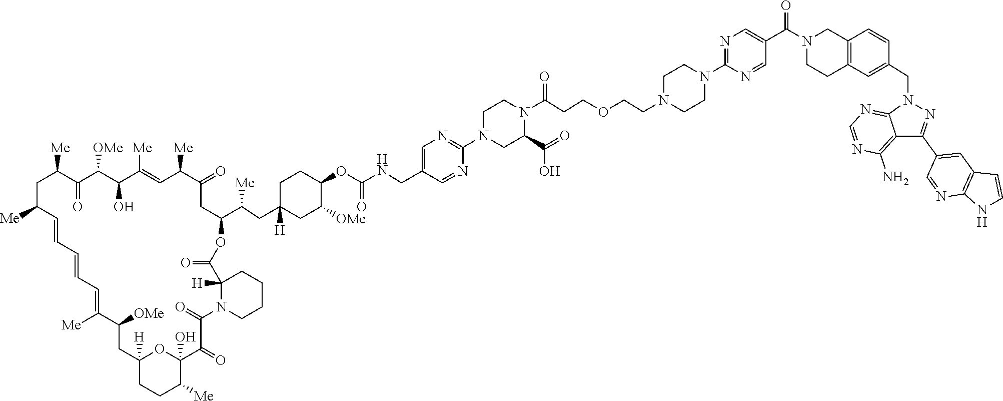 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10cnc%11[nH]ccc%11c%10)c%10c(N)ncnc%109)ccc8C7)cn6)CC5)[C@@H](C(=O)O)C4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 132 | C₉₈H₁₂₈N₁₈O₁₉ | [M + H] = 1861.97 | [M + H] = 1861.9 |
| 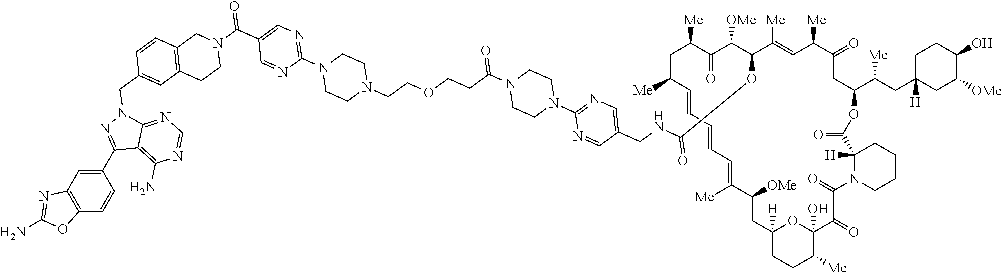 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](OC(=O)NCc2cnc(N3CCN(C(=O)CCOCCN4CCN(c5ncc(C(=O)N6CCc7cc(Cn8nc(-c9ccc%10oc(N)nc%10c9)c9c(N)ncnc98)ccc7C6)cn5)CC4)CC3)nc2)[C@@H](OC)C(=O)[C@H](C)C[C@H](C)/C=C/C=C/C=C/1C] Example 133 | C₉₇H₁₂₈N₁₈O₁₈ | [M + H] = 1833.97 | [M + H] = 1833.9 |
| 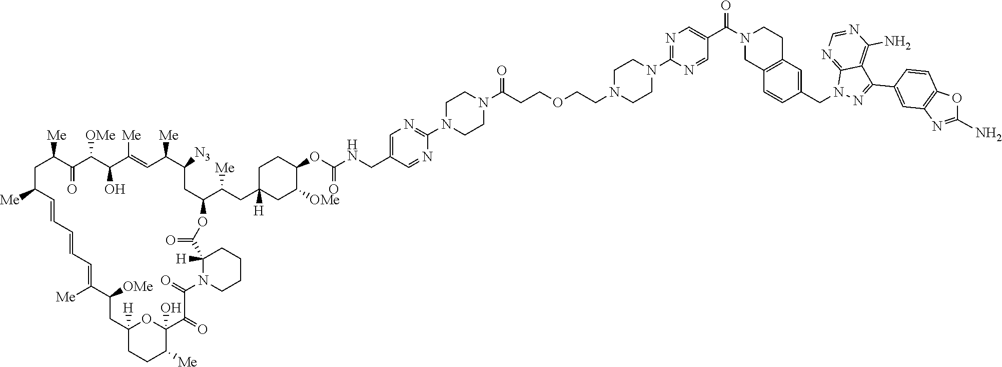 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@H](N=[N+]=[N-])[C@H](C)/C=C(\C)[C@@H](O)[C@@H](OC)C(=O)[C@H](C)C[C@H](C)/C=C/C=C/C=C/1C] Example 151 | C₉₇H₁₂₉N₂₁O₁₇ | [M + H] = 1861.00 | [M + H] = 1860.8 |

Following General Procedure 1, but using the appropriate Intermediates C1 in Table 16 and amine containing pre-linkers in Table 4, the Intermediates E1 in Table 22 were prepared:

TABLE 22
Additional amines prepared

| | Molecular | Calculated | Observed |
|---|---|---|---|
| Structure | Formula | MW | MW |
| 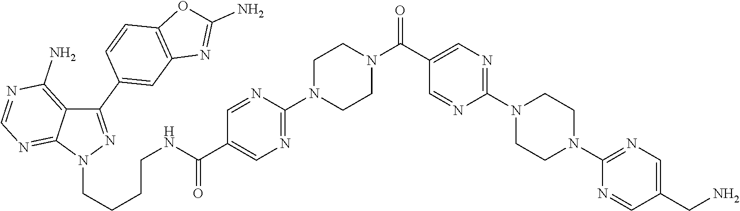 [SMILES: NCc1cnc(N2CCN(c3ncc(C(=O)N4CCN(c5ncc(C(=O)NCCCCn6nc(-c7ccc8oc(N)nc8c7)c7c(N)ncnc76)cn5)CC4)cn3)CC2)nc1] Intermediate E1-1 | C₃₉H₄₃N₁₉O₃ | [M + H] = 826.39 | [M + H] = 826.5 |
| 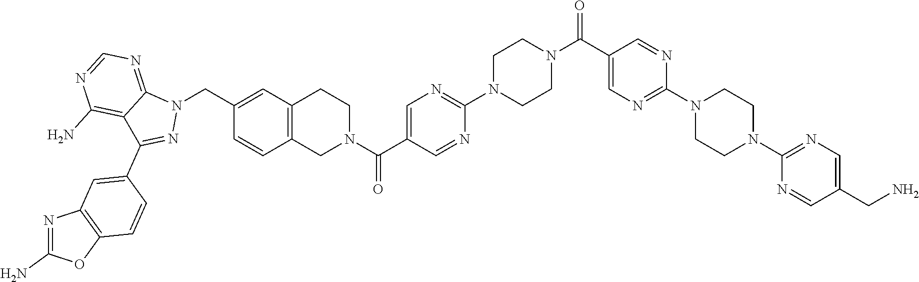 [SMILES: NCc1cnc(N2CCN(c3ncc(C(=O)N4CCN(c5ncc(C(=O)N6CCc7cc(Cn8nc(-c9ccc%10oc(N)nc%10c9)c9c(N)ncnc98)ccc7C6)cn5)CC4)cn3)CC2)nc1] Intermediate E1-2 | C₄₅H₄₅N₁₉O₃ | [M + H] = 900.41 | [M + H] = 900.2 |
| 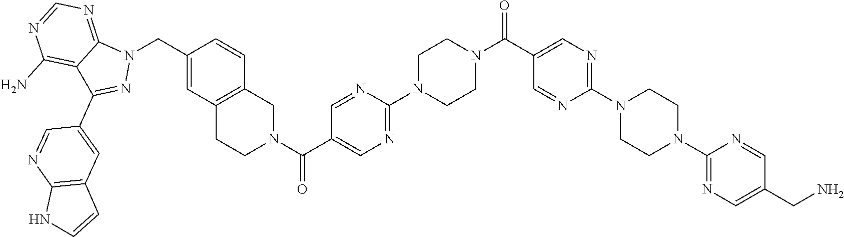 [SMILES: NCc1cnc(N2CCN(c3ncc(C(=O)N4CCN(c5ncc(C(=O)N6CCc7cc(Cn8nc(-c9cnc%10[nH]ccc%10c9)c9c(N)ncnc98)ccc7C6)cn5)CC4)cn3)CC2)nc1] Intermediate E1-3 | C₄₅H₄₅N₁₉O₂ | [M + H] = 884.41 | [M + H] = 884.4 |
| 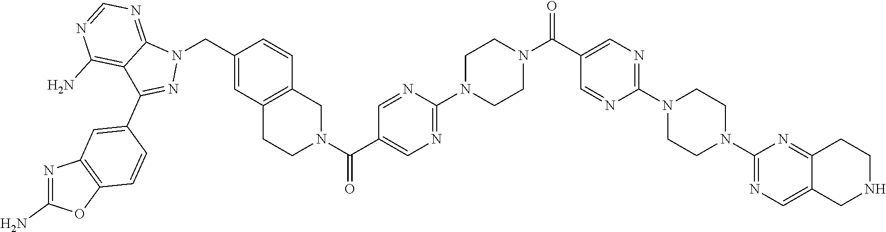 [SMILES: Nc1nc2cc(-c3nn(Cc4ccc5c(c4)CCN(C(=O)c4cnc(N6CCN(C(=O)c7cnc(N8CCN(c9ncc%10c(n9)CCNC%10)CC8)nc7)CC6)nc4)C5)c4ncnc(N)c34)ccc2o1] Intermediate E1-4 | C₄₇H₄₇N₁₉O₃ | [M + H] = 926.42 | [M + H] = 926.6 |
| 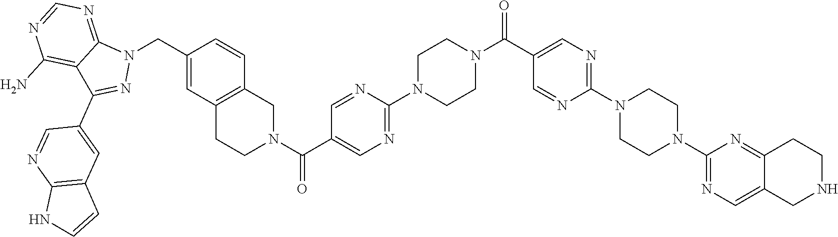 [SMILES: Nc1ncnc2c1c(-c1cnc3[nH]ccc3c1)nn2Cc1ccc2c(c1)CCN(C(=O)c1cnc(N3CCN(C(=O)c4cnc(N5CCN(c6ncc7c(n6)CCNC7)CC5)nc4)CC3)nc1)C2] Intermediate E1-5 | C₄₇H₄₇N₁₉O₂ | [M + H] = 910.43 | [M + H] = 910.2 |
| 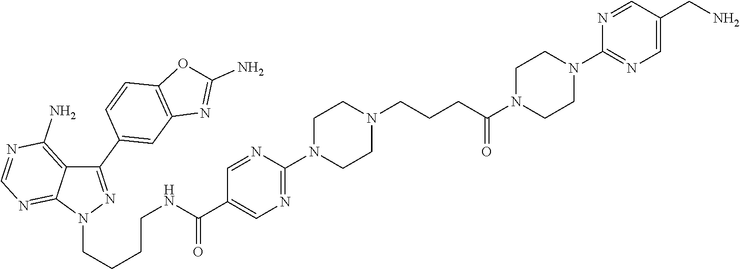 [SMILES: NCc1cnc(N2CCN(C(=O)CCCN3CCN(c4ncc(C(=O)NCCCCn5nc(-c6ccc7oc(N)nc7c6)c6c(N)ncnc65)cn4)CC3)CC2)nc1] Intermediate E1-6 | C₃₈H₄₇N₁₇O₃ | [M + H] = 790.41 | [M + H] = 790.4 |
| 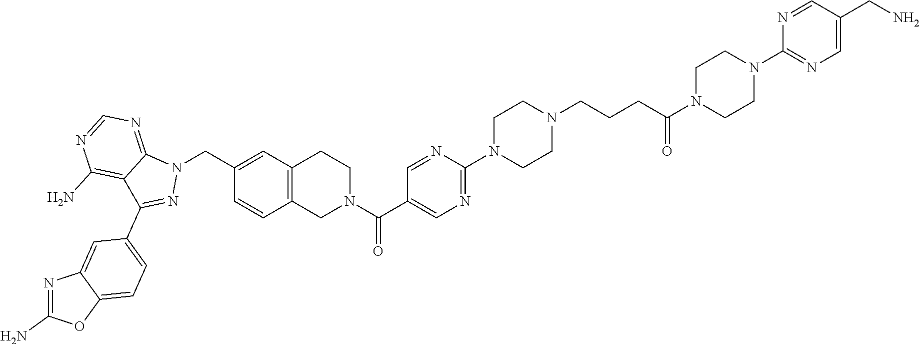 [SMILES: NCc1cnc(N2CCN(C(=O)CCCN3CCN(c4ncc(C(=O)N5CCc6cc(Cn7nc(-c8ccc9oc(N)nc9c8)c8c(N)ncnc87)ccc6C5)cn4)CC3)CC2)nc1] Intermediate E1-7 | C₄₄H₄₉N₁₇O₃ | [M + H] = 864.43 | [M + H] = 864.3 |
| 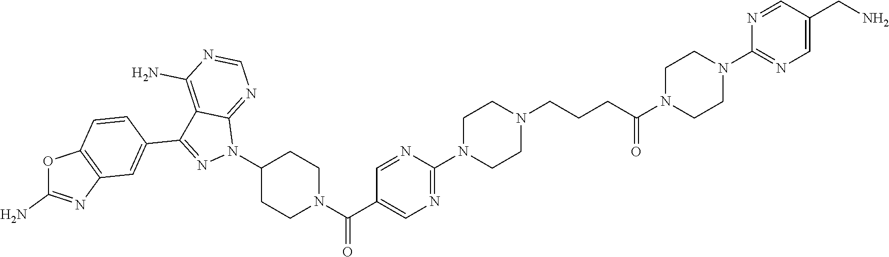 [SMILES: NCc1cnc(N2CCN(C(=O)CCCN3CCN(c4ncc(C(=O)N5CCC(n6nc(-c7ccc8oc(N)nc8c7)c7c(N)ncnc76)CC5)cn4)CC3)CC2)nc1] Intermediate E1-8 | C₃₉H₄₇N₁₇O₃ | [M + H] = 802.41 | [M + H] = 802.3 |

Following General Procedure 2, but using the appropriate 4-nitrophenyl carbonate containing rapamycin monomer in Table 1 and Intermediates E1 from Table 22, the Series 5 bivalent analogs in Table 23 were synthesized:

TABLE 23
Series 5 Bivalent Compounds

| | Molecular | Calculated | Observed |
|---|---|---|---|
| Structure | Formula | MW | MW |
| 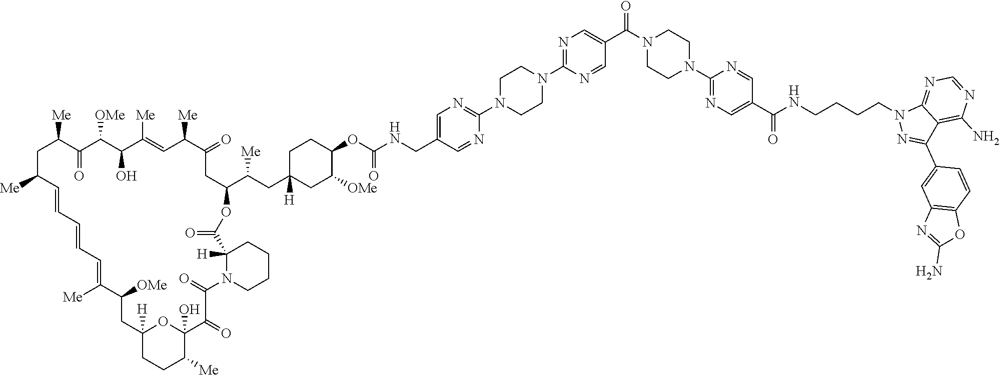 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 59 | C₉₁H₁₂₀N₂₀O₁₇ | [M + H] = 1765.92 | [M + H] = 1765.9 |
| 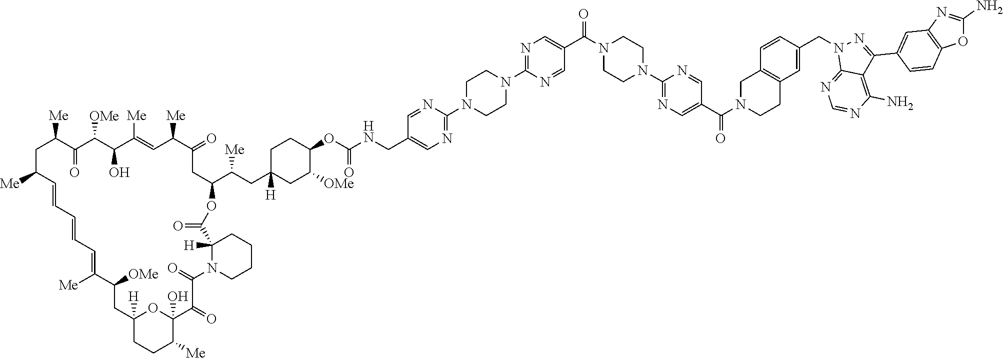 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11ccc%12oc(N)nc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 60 | C₉₇H₁₂₂N₂₀O₁₇ | [M + H] = 1839.94 | [M + H] = 1840.0 |
| 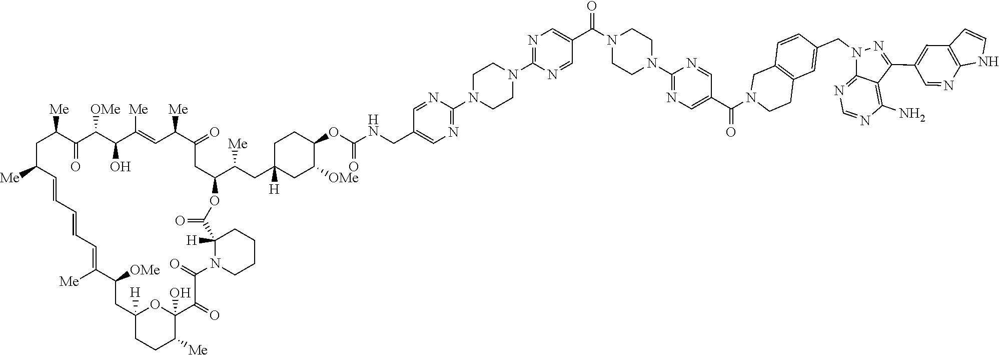 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11cnc%12[nH]ccc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 61 | C₉₇H₁₂₂N₂₀O₁₆ | [M + H] = 1823.94 | [M + H] = 1823.9 |
| 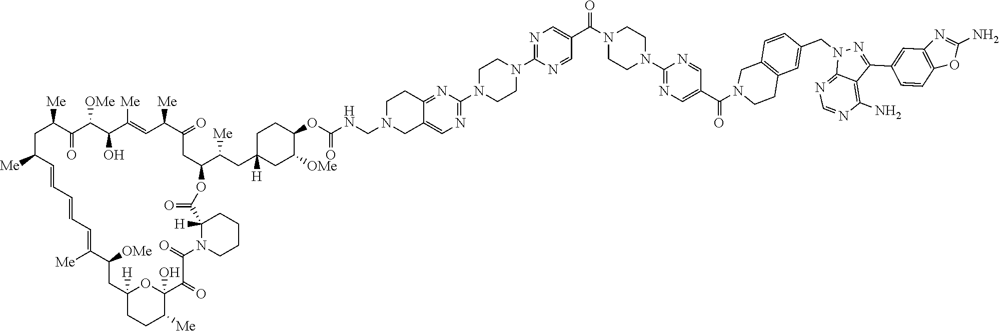 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCN3CCc4nc(N5CCN(c6ncc(C(=O)N7CCN(c8ncc(C(=O)N9CCc%10cc(Cn%11nc(-c%12ccc%13oc(N)nc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 62 | C₉₉H₁₂₄N₂₀O₁₇ | [M + H] = 1865.95 | [M + H] = 1865.8 |
| 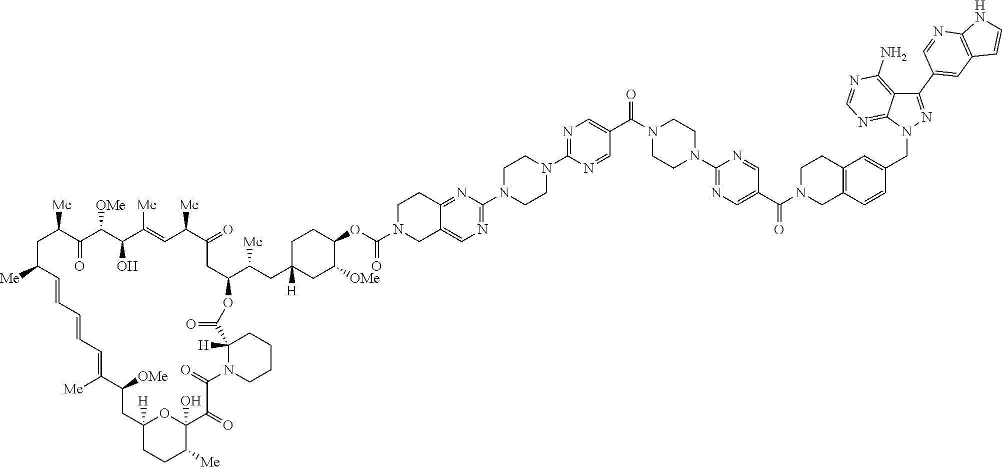 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)N7CCN(c8ncc(C(=O)N9CCc%10cc(Cn%11nc(-c%12cnc%13[nH]ccc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 134 | C₉₉H₁₂₄N₂₀O₁₆ | [M + H] = 1849.96 | [M + H] = 1850.0 |

TABLE 24
Additional amines prepared

| | Molecular | Calculated | Observed |
|---|---|---|---|
| Structure | Formula | MW | MW |
| 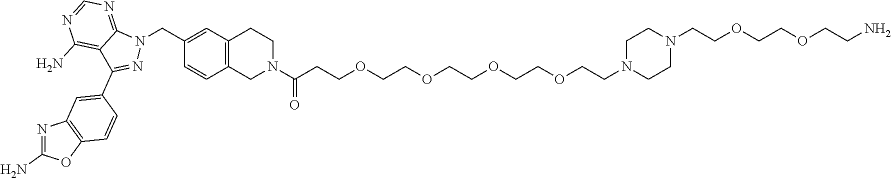 [SMILES: NCCOCCOCCN1CCN(CCOCCOCCOCCOCCC(=O)N2CCc3cc(Cn4nc(-c5ccc6oc(N)nc6c5)c5c(N)ncnc54)ccc3C2)CC1] Intermediate F1-1 | C₄₃H₆₁N₁₁O₈ | [M + H] = 860.48 | [M + H] = 860.4 |
| 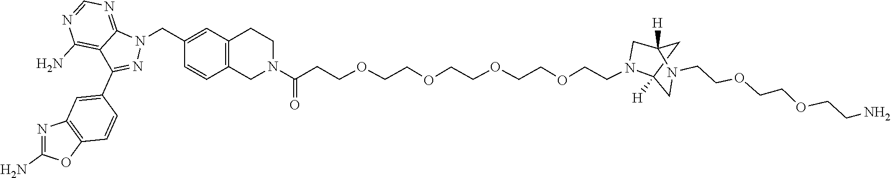 Intermediate F1-2 | C₄₄H₆₁N₁₁O₈ | [M + H] = 872.48 | [M + H] = 872.2 |

Following General Procedure 2, but using the appropriate 4-nitrophenyl carbonate containing rapamycin monomer in Table 1 and Intermediates F1 from Table 24, the Series 6 bivalent analogs in Table 25 were synthesized:

TABLE 25
Series 6 Bivalent Compounds

| | Molecular | Calculated | Observed |
|---|---|---|---|
| Structure | Formula | MW | MW |
| 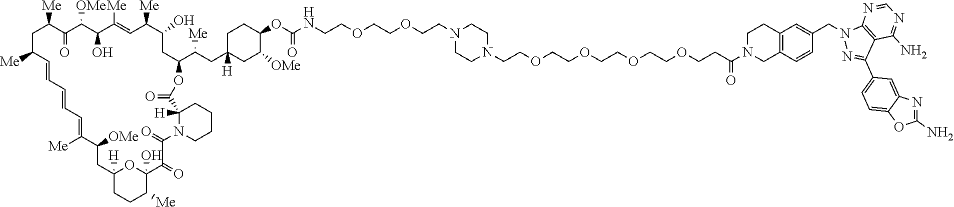 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCN3CCN(CCOCCOCCOCCOCCC(=O)N4CCc5cc(Cn6nc(-c7ccc8oc(N)nc8c7)c7c(N)ncnc76)ccc5C4)CC3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 135 | C₉₅H₁₄₀N₁₂O₂₂ | [M + H] = 1802.03 | [M + H] = 1802.5 |
| 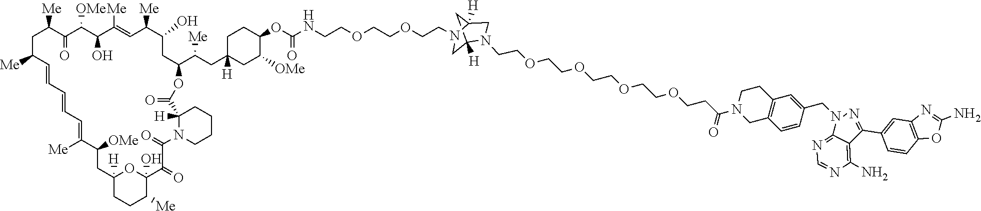 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCOCCN3C[C@@H]4C[C@H]3CN4CCOCCOCCOCCOCCC(=O)N3CCc4cc(Cn5nc(-c6ccc7oc(N)nc7c6)c6c(N)ncnc65)ccc4C3)[C@H](OC)C2)C[C@@H](O)[C@H](C)/C=C(\C)[C@@H](O)[C@@H](OC)C(=O)[C@H](C)C[C@H](C)/C=C/C=C/C=C/1C] Example 152 | C₉₆H₁₄₀N₁₂O₂₂ | [M + H] = 1814.03 | [M + H] = 1813.9 |

Following General Procedure 1, but using the appropriate Intermediates A1 in Table 5 and amine containing pre-linkers in Table 4, the Intermediates G1 in Table 26 were prepared:

TABLE 26
Additional amines prepared

| | Molecular | Calculated | Observed |
|---|---|---|---|
| Structure | Formula | MW | MW |
| 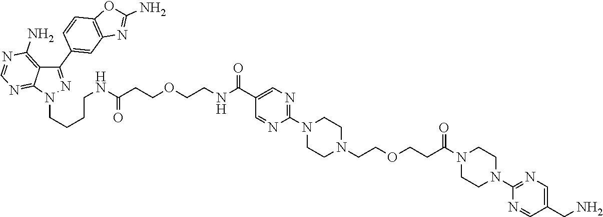 [SMILES: NCc1cnc(N2CCN(C(=O)CCOCCN3CCN(c4ncc(C(=O)NCCOCCC(=O)NCCCCn5nc(-c6ccc7oc(N)nc7c6)c6c(N)ncnc65)cn4)CC3)CC2)nc1] Intermediate G1-1 | C₄₄H₅₈N₁₈O₆ | [M + H] = 935.49 | [M + H] = 935.5 |
| 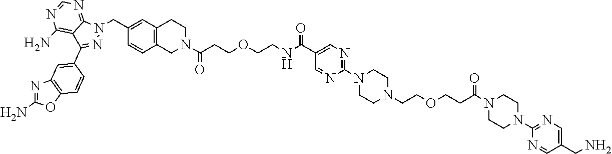 [SMILES: NCc1cnc(N2CCN(C(=O)CCOCCN3CCN(c4ncc(C(=O)NCCOCCC(=O)N5CCc6cc(Cn7nc(-c8ccc9oc(N)nc9c8)c8c(N)ncnc87)ccc6C5)cn4)CC3)CC2)nc1] Intermediate G1-2 | C₅₀H₆₀N₁₈O₆ | [M + H] = 1009.50 | [M + H] = 1009.5 |
| 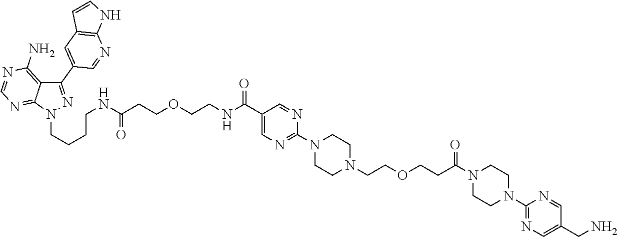 [SMILES: NCc1cnc(N2CCN(C(=O)CCOCCN3CCN(c4ncc(C(=O)NCCOCCC(=O)NCCCCn5nc(-c6cnc7[nH]ccc7c6)c6c(N)ncnc65)cn4)CC3)CC2)nc1] Intermediate G1-3 | C₄₄H₅₈N₁₈O₅ | [M + H] = 919.49 | [M + H] = 919.5 |

Following General Procedure 6, but using the appropriate Intermediates B3 in Table 10 and amine containing pre-linkers in Table 4, the Intermediates G1 in Table 27 were prepared:

TABLE 27
Additional amines prepared

| | Molecular | Calculated | Observed |
|---|---|---|---|
| Structure | Formula | MW | MW |
| 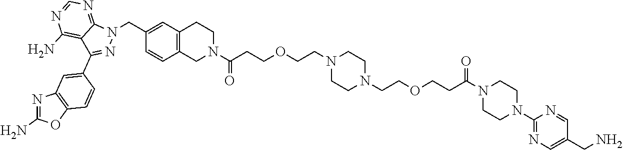 [SMILES: NCc1cnc(N2CCN(C(=O)CCOCCN3CCN(CCOCCC(=O)N4CCc5cc(Cn6nc(-c7ccc8oc(N)nc8c7)c7c(N)ncnc76)ccc5C4)CC3)CC2)nc1] Intermediate G1-4 | C₄₅H₅₇N₁₅O₅ | [M + H] = 888.48 | [M + H] = 888.4 |

Following General Procedure 2, but using the appropriate 4-nitrophenyl carbonate containing rapamycin monomer in Table 1 and Intermediates G1 from Tables 26 and 27, the Series 7 bivalent analogs in Table 28 were synthesized:

TABLE 28
Series 7 Bivalent Compounds

| | | Calcu- | Ob- |
|---|---|---|---|
| | Molecular | lated | served |
| Structure | Formula | MW | MW |
| 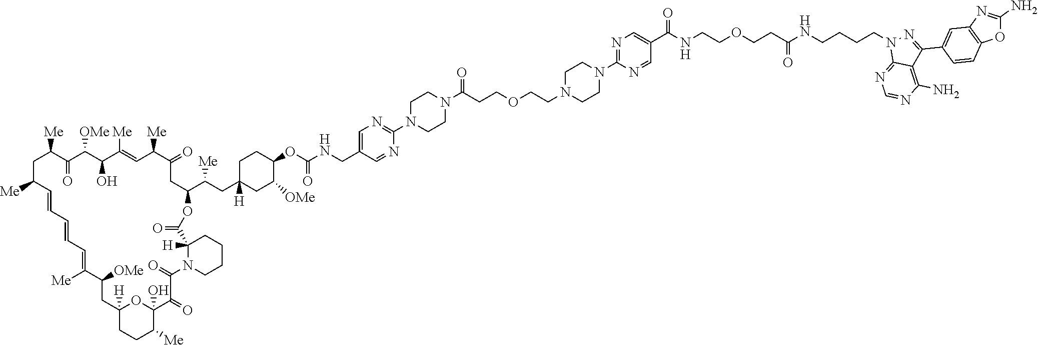 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)NCCCCn7nc(-c8ccc9oc(N)nc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 63 | C₉₆H₁₃₅N₁₉O₂₀ | [M + H] = 1875.02 | [M + H] = 1874.9 |
| 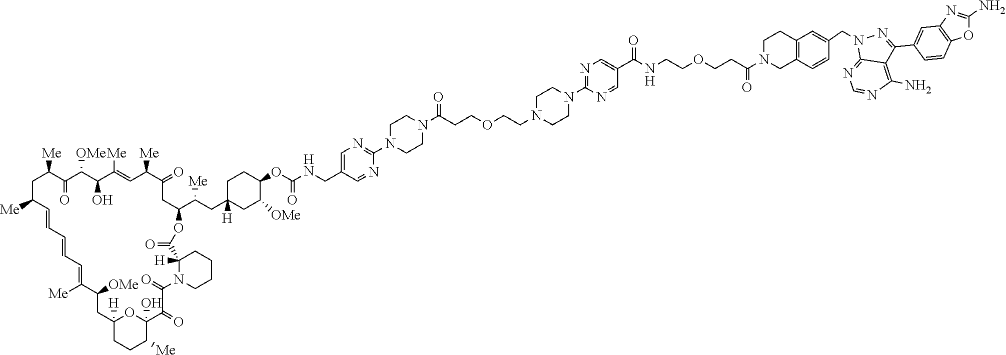 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 64 | C₁₀₂H₁₃₇N₁₉O₂₀ | [M H] = 1949.04 | [M + H] = 1949.0 |
| 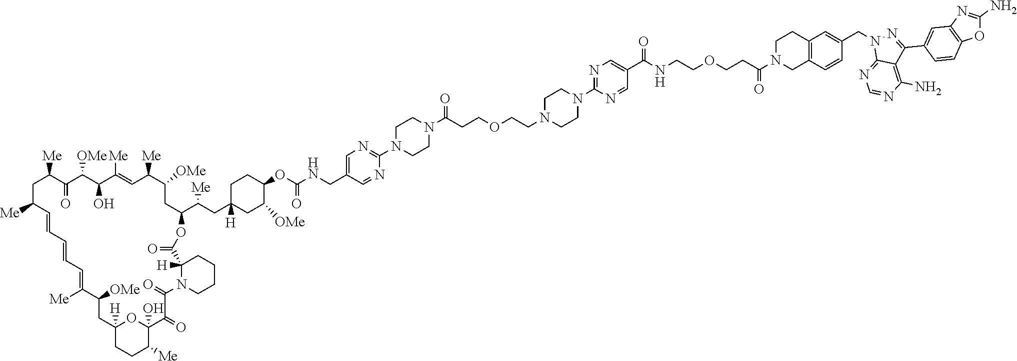 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] Example 65 | C₁₀₃H₁₄₁N₁₉O₂₀ | [M + H] = 1965.07 | [M + H] = 1965.0 |
| 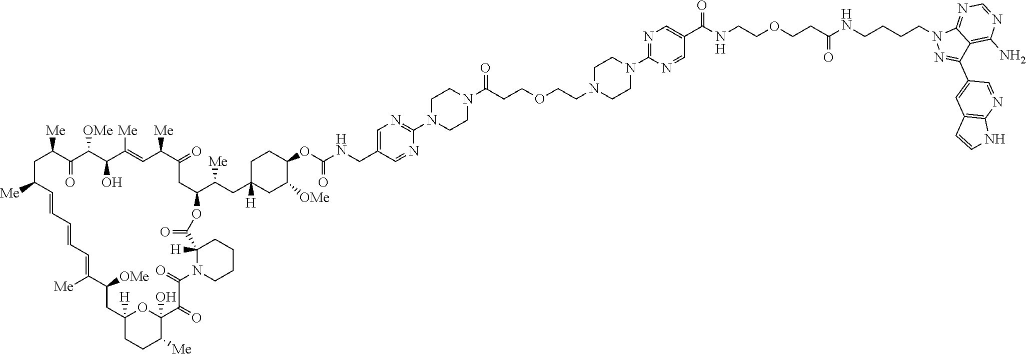 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(C(=O)CCOCCN5CCN(c6ncc(C(=O)NCCOCCC(=O)NCCCCn7nc(-c8cnc9[nH]ccc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 66 | C₉₆H₁₃₅N₁₉O₁₉ | [M + H] = 1859.03 | [M + H] = 1859.0 |

Following General Procedure 1, but using the appropriate Intermediates D1 in Tables 19 and 20 and PEG carboxylic acids, the Intermediates H1 in Table 29 were prepared:

TABLE 29
Additional amines prepared

| | Molecular | Calculated | Observed |
|---|---|---|---|
| Structure | Formula | MW | MW |
| 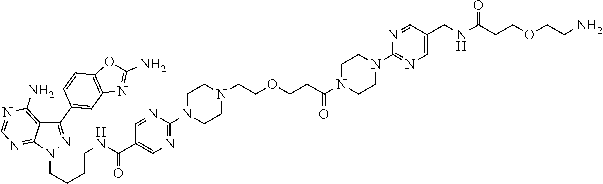 [SMILES: NCCOCCC(=O)NCc1cnc(N2CCN(C(=O)CCOCCN3CCN(c4ncc(C(=O)NCCCCn5nc(-c6ccc7oc(N)nc7c6)c6c(N)ncnc65)cn4)CC3)CC2)nc1] Intermediate H1-1 | C₄₄H₅₈N₁₈O₆ | [M + H] = 935.49 | [M + H] = 935.5 |
| 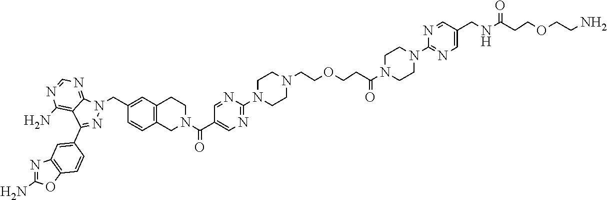 [SMILES: NCCOCCC(=O)NCc1cnc(N2CCN(C(=O)CCOCCN3CCN(c4ncc(C(=O)N5CCc6cc(Cn7nc(-c8ccc9oc(N)nc9c8)c8c(N)ncnc87)ccc6C5)cn4)CC3)CC2)nc1] Intermediate H1-2 | C₅₀H₆₀N₁₈O₆ | [M + 2H]/2 = 505.23 | [M + 2H]/2 = 505.4 |
| 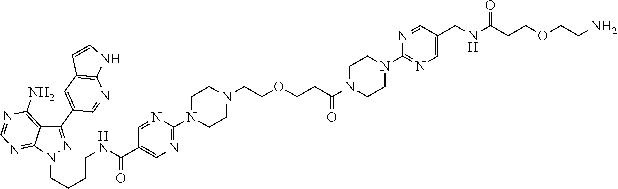 [SMILES: NCCOCCC(=O)NCc1cnc(N2CCN(C(=O)CCOCCN3CCN(c4ncc(C(=O)NCCCCn5nc(-c6cnc7[nH]ccc7c6)c6c(N)ncnc65)cn4)CC3)CC2)nc1] Intermediate H1-3 | C₄₄H₅₈N₁₈O₅ | [M + 2H]/2 = 460.25 | [M + 2H]/2 = 460.3 |

Following General Procedure 2, but using the appropriate 4-nitrophenyl carbonate containing rapamycin monomer in Table 1 and Intermediates H1 from Table 29, the Series 8 bivalent analogs in Table 30 were synthesized:

TABLE 30
Series 8 Bivalent Compounds:

| | Molecular | Calculated | Observed |
|---|---|---|---|
| Structure | Formula | MW | MW |
| 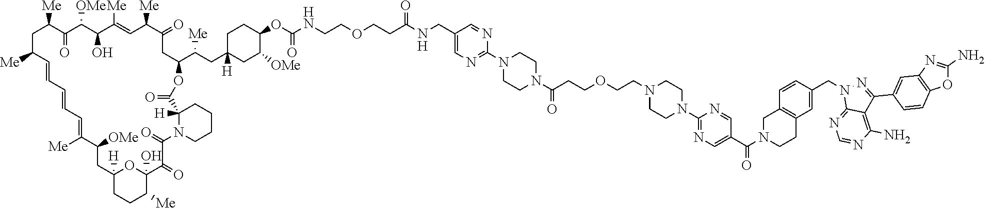 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)N7CCc8cc(Cn9nc(-c%10ccc%11oc(N)nc%11c%10)c%10c(N)ncnc%109)ccc8C7)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 67 | C₁₀₂H₁₃₇N₁₉O₂₀ | [M + H] = 1949.04 | [M + H] = 1949.0 |
| 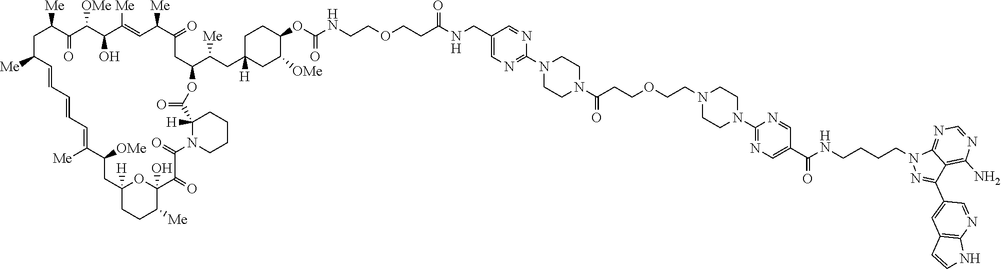 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCCOCCC(=O)NCc3cnc(N4CCN(C(=O)CCOCCN5CCN(c6ncc(C(=O)NCCCCn7nc(-c8cnc9[nH]ccc9c8)c8c(N)ncnc87)cn6)CC5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C] Example 68 | C₉₆H₁₃₅N₁₉O₁₉ | [M + H] = 1859.03 | [M + H] = 1859.0 |
| 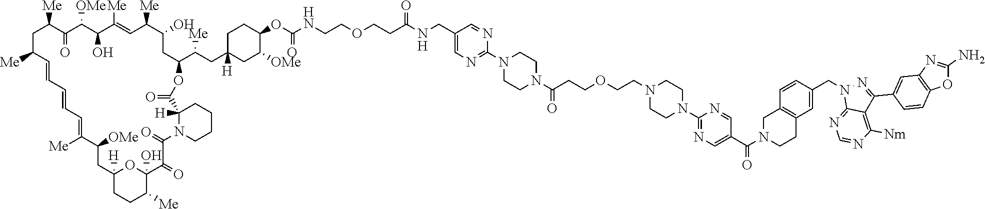 Example 69 | C₁₀₂H₁₃₉N₁₉O₂₀ | [M + H] = 1951.05 | [M + H] = 1951.1 |
| 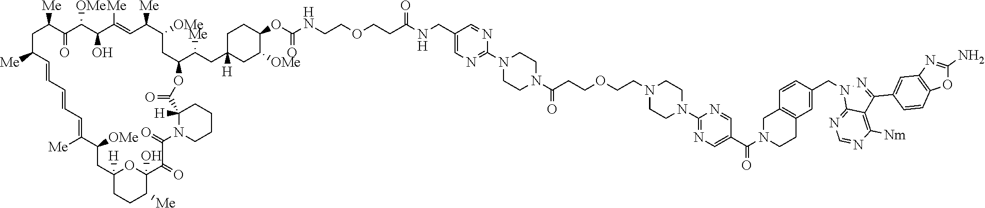 Example 70 | C₁₀₃H₁₄₁N₁₉O₂₀ | [M + H] = 1965.07 | [M + H] = 1965.1 |

### BIOLOGICAL EXAMPLES

### Cell Based AlphaLISA Assays for Determining IC50 for Inhibition of P-Akt (S473), P-4E-BP1 (T37/46), and P-P70S6K (T389) in MDA-MB-468 Cells

mTOR Kinase Cellular Assay

To measure functional activity of mTORC1 and mTORC2 in cells the phosphorylation of 4EBP1 (Thr37/46) and P70S6K (Thr389), and AKT1/2/3 (Ser473) was monitored using AlphaLisa SureFire Ultra Kits (Perkin Elmer). MDA-MB-468 cells (ATCC® HTB-132) were cultured in 96-well tissue culture plates and treated with compounds in the disclosure at concentrations varying from 0.017-1,000 nM for two to four hours at 37° C. Incubations were terminated by removal of the assay buffer and addition of lysis buffer provided with the assay kit. Samples were processed according to the manufacturer's instructions. The Alpha signal from the respective phosphoproteins was measured in duplicate using a microplate reader (Envision, Perkin-Elmer or Spectramax M5, Molecular Devices). Inhibitor concentration response curves were analyzed using normalized IC₅₀ regression curve fitting with control based normalization.

As an example, measured IC₅₀ values for selected compounds are reported below:

| | IC₅₀ for Inhibition of mTORC1 and | | |
|---|---|---|---|
| | mTORC2 Substrate Phosphorylation (nM) | | |
| | p-P70S6K- | p-4E-BP1- | p-AKT1/2/3- |
| Compound | (T389) | (T37/46) | (S473) |
| MLN-128 | 1.4 | 16 | 3.7 |
| Rapamycin | 0.2 | >1,000 | >1,000 |

As an example, measured pIC₅₀ values for selected compounds are reported below:

| | pIC₅₀ for Inhibition of mTORC1 and | | |
|---|---|---|---|
| | mTORC2 Substrate Phosphorylation | | |
| | p-P70S6K- | p-4E-BP1- | p-AKT1/2/3- |
| Example | (T389) | (T37/46) | (S473) |
| 1 | +++ | +++ | ++ |
| 2 | +++ | +++ | ++ |
| 3 | +++ | +++ | ++ |
| 4 | +++ | +++ | ++ |
| 5 | +++ | +++ | + |
| 6 | +++ | +++ | ++ |
| 7 | +++ | +++ | ++ |
| 8 | +++ | +++ | ++ |
| 9 | +++ | +++ | ++ |
| 10 | +++ | − | − |
| 11 | +++ | +++ | ++ |
| 12 | +++ | +++ | + |
| 13 | +++ | +++ | ++ |
| 14 | ++ | +++ | ++ |
| 15 | +++ | +++ | ++ |
| 16 | +++ | +++ | +++ |
| 17 | +++ | +++ | ++ |
| 18 | +++ | ++ | + |
| 19 | +++ | +++ | +++ |
| 20 | +++ | +++ | +++ |
| 21 | +++ | +++ | + |
| 27 | +++ | +++ | ++ |
| 28 | +++ | +++ | − |
| 29 | +++ | +++ | − |
| 30 | +++ | +++ | ++ |
| 31 | +++ | +++ | ++ |
| 32 | +++ | +++ | ++ |
| 33 | +++ | +++ | − |
| 34 | +++ | − | − |
| 35 | +++ | +++ | ++ |
| 36 | +++ | +++ | − |
| 37 | +++ | +++ | − |
| 38 | +++ | − | − |
| 39 | +++ | +++ | +++ |
| 40 | +++ | +++ | ++ |
| 41 | +++ | +++ | − |
| 42 | +++ | +++ | ++ |
| 43 | +++ | +++ | ++ |
| 46 | +++ | +++ | ++ |
| 47 | +++ | +++ | ++ |
| 48 | +++ | +++ | ++ |
| 49 | +++ | +++ | ++ |
| 50 | +++ | +++ | + |
| 51 | +++ | +++ | +++ |
| 52 | +++ | +++ | − |
| 53 | +++ | +++ | ++ |
| 54 | +++ | +++ | +++ |
| 55 | +++ | +++ | ++ |
| 56 | +++ | +++ | − |
| 59 | +++ | +++ | +++ |
| 60 | +++ | ++ | + |
| 61 | +++ | − | − |
| 62 | ++ | − | − |
| 63 | +++ | +++ | +++ |
| 64 | +++ | +++ | ++ |
| 65 | +++ | +++ | + |
| 66 | +++ | +++ | ++ |
| 67 | + | + | + |
| 68 | + | + | − |
| 69 | +++ | +++ | ++ |
| 70 | +++ | +++ | + |
| 71 | +++ | +++ | ++ |
| 72 | +++ | +++ | +++ |
| 73 | +++ | +++ | + |
| 74 | +++ | +++ | + |
| 75 | +++ | +++ | ++ |
| 76 | +++ | +++ | ++ |
| 77 | +++ | ++ | + |
| 78 | +++ | +++ | +++ |
| 79 | +++ | +++ | + |
| 80 | +++ | +++ | +++ |
| 81 | +++ | ++ | − |
| 82 | +++ | +++ | − |
| 83 | +++ | +++ | ++ |
| 84 | +++ | +++ | ++ |
| 85 | +++ | +++ | ++ |
| 86 | +++ | +++ | ++ |
| 87 | +++ | +++ | ++ |
| 88 | +++ | +++ | ++ |
| 89 | +++ | +++ | − |
| 90 | +++ | − | − |
| 91 | +++ | +++ | ++ |
| 92 | +++ | +++ | ++ |
| 93 | +++ | +++ | ++ |
| 94 | +++ | +++ | +++ |
| 95 | +++ | +++ | ++ |
| 96 | +++ | +++ | + |
| 97 | +++ | +++ | ++ |
| 98 | +++ | +++ | ++ |
| 99 | +++ | +++ | − |
| 100 | +++ | +++ | ++ |
| 101 | +++ | +++ | ++ |
| 102 | +++ | +++ | ++ |
| 103 | +++ | − | − |
| 104 | +++ | +++ | − |
| 105 | +++ | ++ | − |
| 106 | +++ | ++ | − |
| 107 | +++ | − | − |
| 108 | ++ | − | − |
| 109 | + | − | − |
| 110 | ++ | − | − |
| 111 | +++ | +++ | − |
| 112 | +++ | − | − |
| 113 | +++ | +++ | ++ |
| 114 | +++ | +++ | − |
| 115 | +++ | +++ | +++ |
| 116 | +++ | +++ | − |
| 117 | +++ | +++ | ++ |
| 118 | +++ | +++ | ++ |
| 119 | +++ | +++ | ++ |
| 120 | +++ | +++ | ++ |
| 121 | +++ | +++ | − |
| 122 | +++ | +++ | ++ |
| 123 | +++ | +++ | + |
| 124 | +++ | +++ | ++ |
| 125 | +++ | +++ | ++ |
| 126 | +++ | ++ | − |
| 127 | +++ | ++ | − |
| 128 | +++ | ++ | + |
| 129 | +++ | +++ | − |
| 130 | +++ | +++ | − |
| 131 | +++ | +++ | ++ |
| 132 | +++ | +++ | ++ |
| 133 | +++ | +++ | ++ |
| 134 | +++ | +++ | − |
| 135 | +++ | − | − |
| 136 | +++ | ++ | ++ |
| 137 | +++ | +++ | ++ |
| 138 | +++ | +++ | +++ |
| 139 | +++ | +++ | + |
| 140 | + | + | − |
| 141 | +++ | +++ | + |
| 142 | ++ | − | − |
| 143 | +++ | ++ | − |
| 144 | +++ | +++ | ++ |
| 145 | +++ | ++ | + |
| 146 | − | − | − |
| 147 | +++ | +++ | ++ |
| 148 | +++ | +++ | ++ |
| 149 | +++ | +++ | ++ |
| 150 | +++ | +++ | + |
| 151 | +++ | ++ | − |
| 152 | +++ | +++ | + |

Note:
pIC50 (p-P70S6K-(T389))
≥9 +++
9 > pIC50 ≥ 8 ++
8 > pIC50 ≥ 6 +
<6 −
pIC50 (p-4E-BP1-(T37/46) or p-AKT1/2/3-(S473))
≥8.5 +++
8.5 > pIC50 ≥ 7.5 ++
7.5 > pIC50 ≥ 6.0 +
<6 −

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1-95. (canceled)

96. A compound selected from the group consisting of:

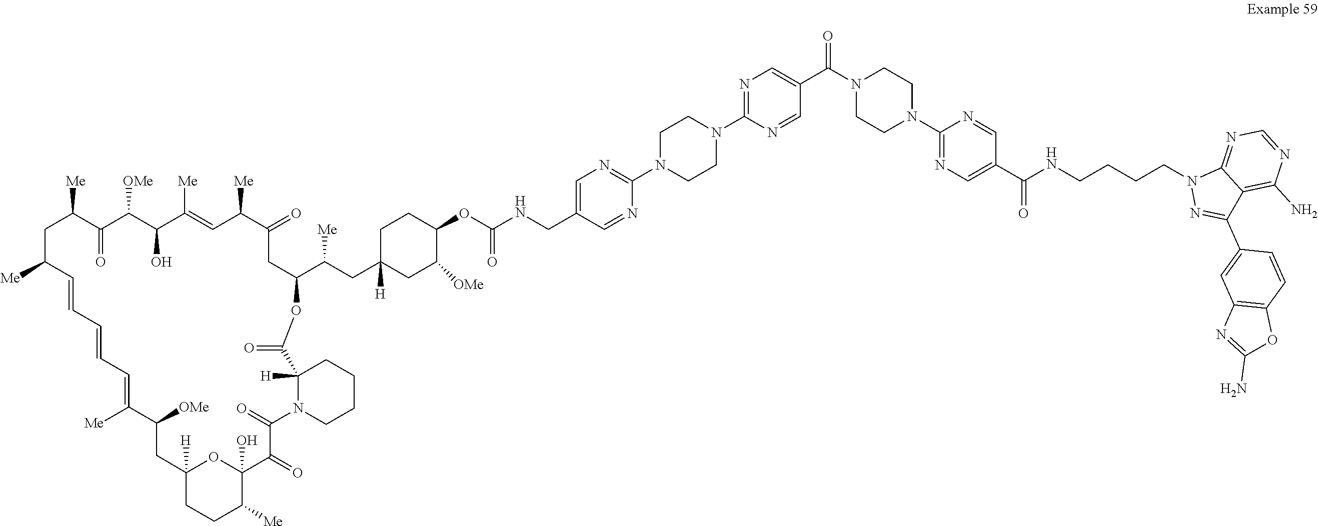 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)NCCCCn8nc(-c9ccc%10oc(N)nc%10c9)c9c(N)ncnc98)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

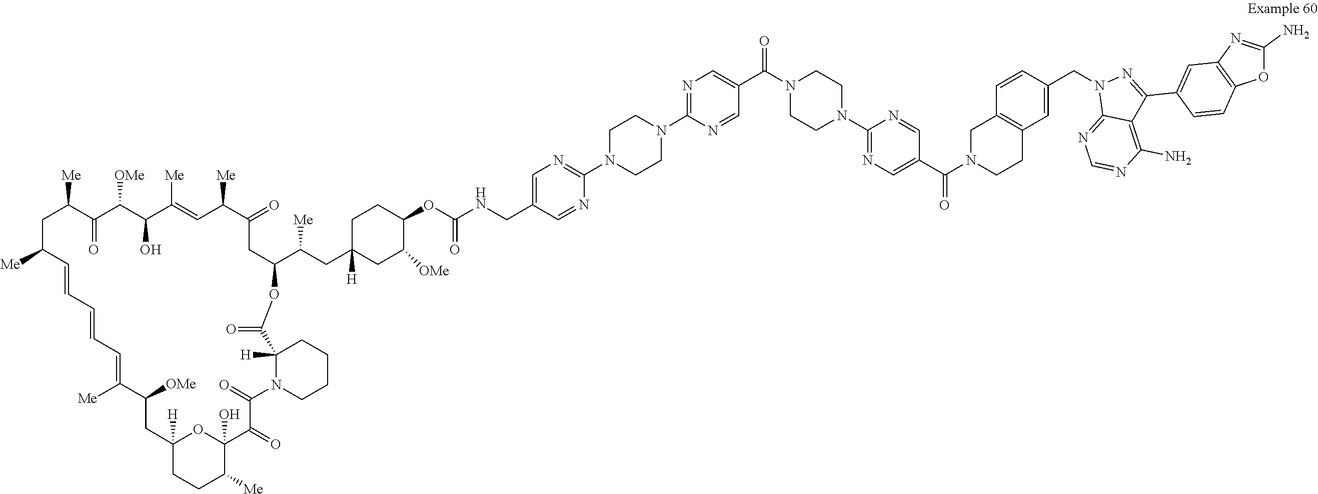 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11ccc%12oc(N)nc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

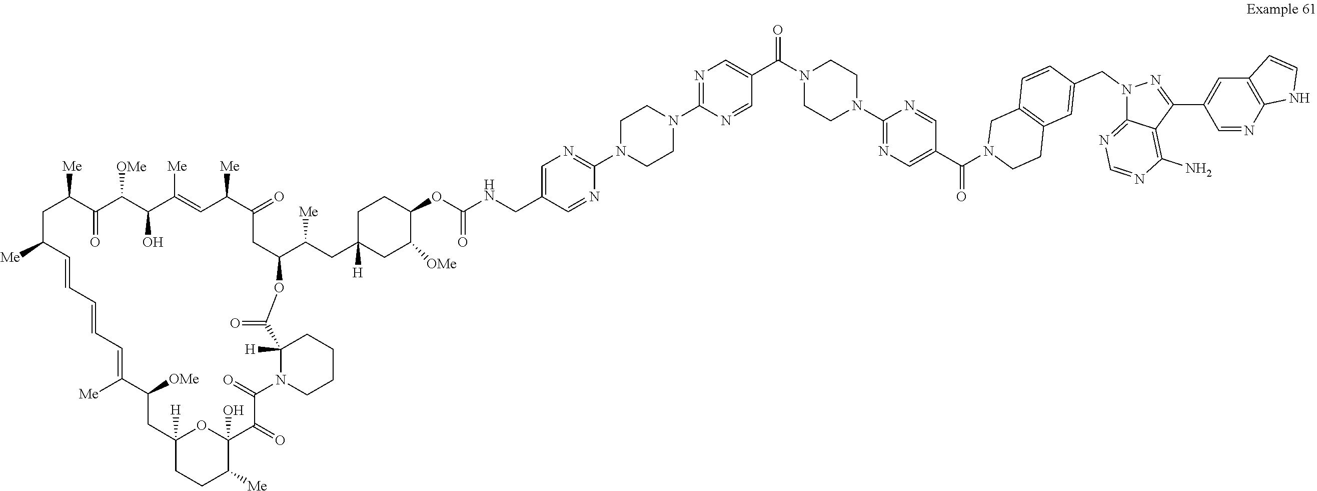 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cnc(N4CCN(c5ncc(C(=O)N6CCN(c7ncc(C(=O)N8CCc9cc(Cn%10nc(-c%11cnc%12[nH]ccc%12c%11)c%11c(N)ncnc%11%10)ccc9C8)cn7)CC6)cn5)CC4)nc3)[C@H](OC)C2)CC(=O)[C@H](C)/C=C(\C)[C@@H](O)[C@@H](OC)C(=O)[C@H](C)C[C@H](C)/C=C/C=C/C=C/1C]

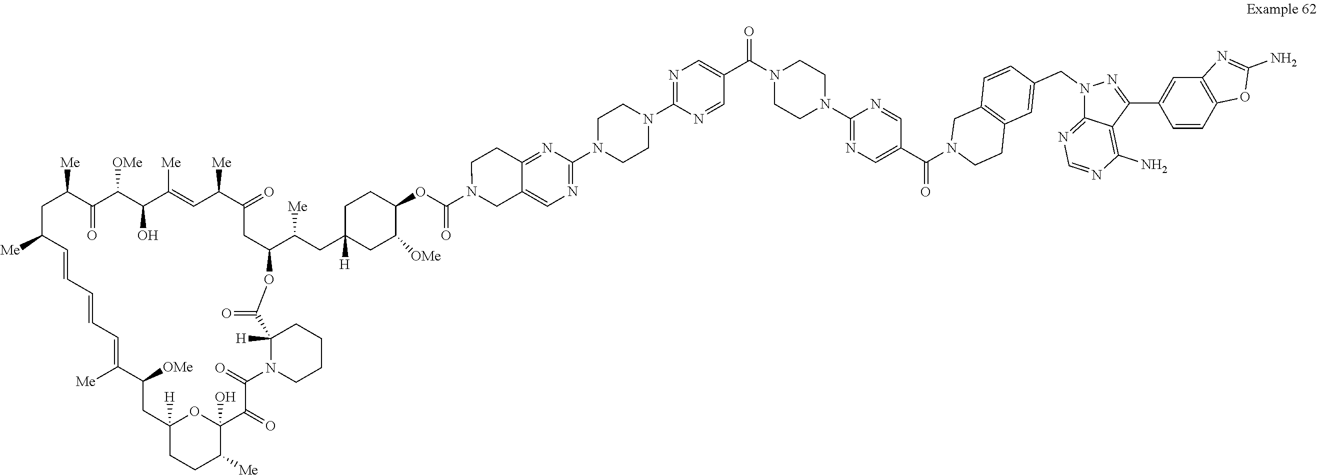 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)N7CCN(c8ncc(C(=O)N9CCc%10cc(Cn%11nc(-c%12ccc%13oc(N)nc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

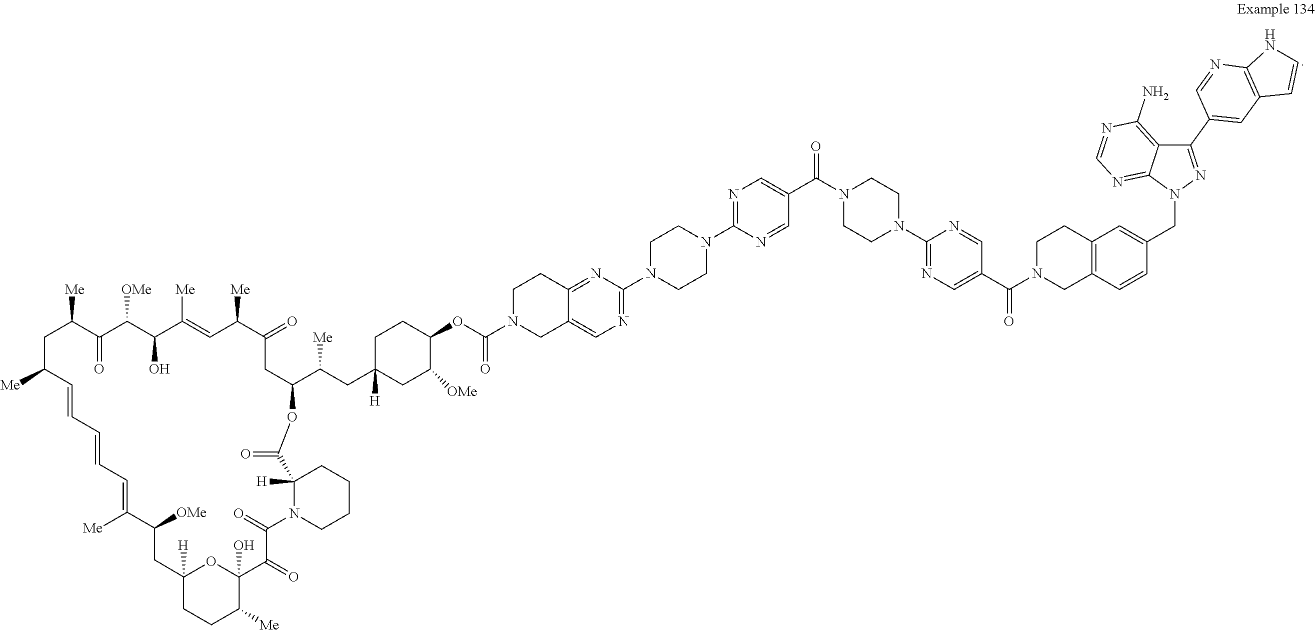 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)N3CCc4nc(N5CCN(c6ncc(C(=O)N7CCN(c8ncc(C(=O)N9CCc%10cc(Cn%11nc(-c%12cnc%13[nH]ccc%13c%12)c%12c(N)ncnc%12%11)ccc%10C9)cn8)CC7)cn6)CC5)ncc4C3)[C@H](OC)C2)CC(=O)[C@H](C)/C=C(\C)[C@@H](O)[C@@H](OC)C(=O)[C@H](C)C[C@H](C)/C=C/C=C/C=C/1C]

97. A pharmaceutical composition comprising a compound of claim 96, or a pharmaceutically acceptable salt thereof, and at least one of a pharmaceutically acceptable carrier, diluent, or excipient.


SEQUENCE LISTING (5 sequences)

SEQ ID NO: 1 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 2 - DNA, 8733 bp, Homo sapiens
  source 1..8733: mol_type = other DNA
gctcccggcttagaggacagcggggaaggcgggcggtggggcagggggcctgaagcggcggtaccggtgctggcggcggcagctgaggccttggccgaagccgcgcgaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacatatgtaaaaaaaaa

SEQ ID NO: 3 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 4 - protein, 118 aa, Homo sapiens
MSGGSSCSQTPSRAIPATRRVVLGDGVQLPPGDYSTTPGGTLFSTTPGGTRIIYDRKFLMECRNSPVTKTPPRDLPTIPGVTSPSSDEPPMEASQSHLRNSPEDKRAGGEESQFEMDI

SEQ ID NO: 5 - protein, 480 aa, Homo sapiens [sequence omitted]